(19) United States
(12) Patent Application Publication
(10) Pub. No.: US 2025/0179524 A1
(43) Pub. Date: Jun. 5, 2025

(54) RECOMBINANT POXVIRUSES FOR CANCER IMMUNOTHERAPY

(71) Applicant: Memorial Sloan-Kettering Cancer Center, New York, NY (US)

(72) Inventors: Liang DENG, New York, NY (US); Jedd WOLCHOK, New York, NY (US); Stewart SHUMAN, New York, NY (US); Taha MERGHOUB, New York, NY (US); Ning YANG, New York, NY (US); Yi WANG, New York, NY (US); Gregory MAZO, New York, NY (US); Peihong DAI, New York, NY (US); Weiyi WANG, New York, NY (US)

(73) Assignee: Memorial Sloan-Kettering Cancer Center, New York, NY (US)

(21) Appl. No.: 19/033,021

(22) Filed: Jan. 21, 2025

Related U.S. Application Data
(62) Division of application No. 17/275,974, filed on Mar. 12, 2021, now Pat. No. 12,252,702.
(60) Provisional application No. 62/731,876, filed on Sep. 15, 2018.
(60) Provisional application No. 62/767,485, filed on Nov. 14, 2018.
(60) Provisional application No. 62/828,975, filed on Apr. 3, 2019.

(51) Int. Cl. C12N 15/86; A61K 9/00; A61K 31/137; A61K 39/00; A61K 39/39; A61K 39/395; A61K 45/06; A61P 35/00; A61P 37/04; C07K 14/54; C07K 14/55; C07K 14/705; C07K 14/71; C07K 16/28; C12N 7/00

(52) U.S. Cl. CPC C12N 15/86; A61K 9/0019; A61K 31/137; A61K 39/39; A61K 39/39558; A61K 45/06; A61P 35/00; A61P 37/04; C07K 14/54; C07K 14/5434; C07K 14/5443; C07K 14/55; C07K 14/70575; C07K 14/71; C07K 16/2818; C12N 7/00; A61K 2039/5256; A61K 2039/54; A61K 2039/545; A61K 2039/55516; A61K 2039/585; C12N 2710/24134; C12N 2710/24143

(57) ABSTRACT

Disclosed herein are methods and compositions related to the treatment, prevention, and/or amelioration of cancer in a subject in need thereof. In particular aspects, the present technology relates to the use of genetically engineered or recombinant poxviruses, including a modified vaccinia Ankara (MVA) virus comprising a deletion of E3L (MVAΔE3L) engineered to express OX40L (MVAΔE3L-OX40L), an MVA virus comprising a deletion of C7L (MVAΔC7L) engineered to express OX40L (MVAΔC7L-OX40L), a MVAΔC7L engineered to express OX40L and human Fms-like tyrosine kinase 3 ligand (hFlt3L) (MVAΔC7L-hFlt3L-OX40L), an MVA comprising a deletion of E5R (MVAΔE5R), a vaccinia virus comprising a deletion of C7L (VACVΔC7L) engineered to express OX40L (VACVΔC7L-OX40L), a VACVΔC7L engineered to express both OX40L and hFlt3L (VACVΔC7L-hFlt3L-OX40L), a VACV comprising a deletion of E5R (VACVΔE5R), a myxoma virus (MYXV) comprising a deletion of M31R (MYXVΔM3IR), or combinations thereof, alone or in combination with other agents, as an oncolytic and immunotherapeutic composition.

### CROSS-REFERENCE TO RELATED APPLICATIONS

This application is a division of U.S. application Ser. No. 17/275,974, filed Mar. 12, 2021, which is a U.S. National Phase Application under 37 U.S.C § 371 of International Application No. PCT/US2019/051343, filed on Sep. 16, 2019, which claims the benefit of and priority to U.S. Provisional Application No. 62/731,876, filed Sep. 15, 2018, U.S. Provisional Application No. 62/767,485, filed Nov. 14, 2018, and U.S. Provisional Application No. 62/828,975, filed Apr. 3, 2019, the disclosures of which are each incorporated herein by reference in their entireties.

### STATEMENT OF FEDERALLY FUNDED RESEARCH

This invention was made with government support under AI073736, AI095692, AR068118, and CA008748 awarded by the National Institutes of Health. The government has certain rights in the invention.

### SEQUENCE LISTING

The instant application contains a Sequence Listing which has been submitted electronically in ASCII format and is hereby incorporated herein by reference in its entirety. Said XML copy, created on Jan. 9, 2025, is named 046139-076D01US.xml and is 3 kb in size.

### TECHNICAL FIELD

The technology of the present disclosure relates generally to the fields of oncology, virology, and immunotherapy. In particular, the present technology relates to the use of poxviruses, including a recombinant modified vaccinia Ankara (MVA) virus comprising a deletion of E3L (MVAΔE3L) genetically engineered to express OX40L (MVAΔE3L-OX40L); a recombinant MVA virus comprising a deletion of C7L (MVAΔC7L) genetically engineered to express OX40L (MVAΔC7L-OX40L); a recombinant MVAΔC7L engineered to express OX40L and hFlt3L (MVAΔC7L-hFlt3L-OX40L); a recombinant MVA genetically engineered to comprise a deletion of C7L, a deletion of E5R, and to express hFlt3L and OX40L (MVAΔC7LΔE5R-hFlt3L-OX40L); a recombinant MVA genetically engineered to comprise a deletion of E5R (MVAΔE5R); a recombinant MVA genetically engineered to comprise a deletion of E5R and to express hFlt3L and OX40L (MVAΔE5R-hFlt3L-OX40L); a recombinant MVA genetically engineered to comprise a deletion of E3L, a deletion of E5R, and to express hFtl3L and OX40L (MVAΔE3LΔE5R-hFlt3L-OX40L); a recombinant MVA genetically engineered to comprise a deletion of E5R, a deletion of C11R, and to express hFlt3L and OX40L (MVAΔE5R-hFlt3L-OX40L-ΔC11R); a recombinant MVA genetically engineered to comprise a deletion of E3L, a deletion of E5R, a deletion of C11R, and to express hFlt3L and OX40L (MVAΔE3LΔE5R-hFlt3L-OX40L-ΔC11R); a recombinant vaccinia virus comprising a deletion of C7L (VACVΔC7L) genetically engineered to express OX40L (VACVΔC7L-OX40L); a recombinant VACVΔC7L genetically engineered to express both OX40L and hFlt3L (VACVΔC7L-hFlt3L-OX40L); a VACV genetically engineered to comprise a deletion of E5R (VACVΔE5R); a recombinant VACV genetically engineered to comprise a deletion of E5R, a deletion of thymidine kinase (TK), and to express anti-CTLA-4, hFlt3L, and OX40L (VACV-TK⁻-anti-CTLA-4-ΔE5R-hFlt3L-OX40L); a VACV genetically engineered to comprise a deletion of B2R (VACVΔB2R); a VACV genetically engineered to comprise an E3LΔ83N deletion and a B2R deletion (VACVE3LΔ83NΔB2R); a VACV genetically engineered to comprise an E5R deletion and a B2R deletion (VACVΔE5RΔB2R); a VACV genetically engineered to comprise an E3LΔ83N deletion, an E5R deletion, and a B2R deletion (VACVE3LΔ83NΔE5RΔB2R); a VACV genetically engineered to comprise an E3LΔ83N deletion, a deletion of thymidine kinase (TK), and an E5R deletion, and expressing anti-CTLA-4, hFlt3L, OX40L, and IL-12 (VACVE3LΔ83N-ΔTK-anti-CTLA-4-ΔE5R-hFlt3L-OX40L-IL-12); a VACV genetically engineered to comprise an E3LΔ83N deletion, a deletion of thymidine kinase (TK), an E5R deletion, and a B2R deletion, and expressing anti-CTLA-4, hFlt3L, OX40L, and IL-12 (VACVE3LΔ83N-ΔTK-anti-CTLA-4-ΔE5R-hFlt3L-OX40L-IL-12-ΔB2R); a MYXV genetically engineered to comprise a deletion of M31R (MYXVΔM31R); a recombinant MYXV genetically engineered to comprise a deletion of M31R and to express hFl3L and OX40L (MYXVΔM31R-hFlt3L-OX40L); a MYXV genetically engineered to comprise a deletion of M63R (MYXVΔM63R); a MYXV genetically engineered to comprise a deletion of M64R (MYXVΔM64R); an MVA genetically engineered to comprise a deletion of WR199 (MVAΔWR199); an MVA genetically engineered to comprise a deletion of E5R, a deletion of WR199, and expressing hFlt3L and OX40L (MVAΔE5R-hFlt3L-OX40L-ΔWR199); or combinations thereof, alone or in combination with immune checkpoint blocking agents, immunomodulatory agents, and/or anti-cancer drugs as an immunotherapeutic and/or oncolytic composition. In some embodiments, the technology of the present disclosure relates to any one of the foregoing viruses further modified to express a specific gene of interest (SG), such as genes encoding any one or more of the following immunomodulatory proteins, including but not limited to hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L. In some embodiments, the virus backbones are further modified to comprise deletions or mutations of genes, including but not limited to thymidine kinase (TK), E3L (ΔE3L), E3LΔ83N, B2R (ΔB2R), B19R (B18R; ΔWR200), E5R, K7R, C12L (IL18BP), B8R, B14R, N1L, C11R, K1L, M1L, N2L, and/or WR199. In some embodiments, the technology of the present disclosure relates to the use of any one of the foregoing viruses as a vaccine adjuvant. In particular, the present technology relates to the use of MVAΔC7L-hFlt3L-TK(−)-OX40L, MVAΔE5R-hFlt3L-OX40L, MVAΔC7LΔE5R-hFlt3L-OX40L, and Heat-inactivated MVAΔE5R as a vaccine adjuvant for tumor antigens in cancer vaccines alone or in combination with immune checkpoint blockade (ICB) antibodies for use as a cancer immunotherapeutic. In some embodiments, the technology of the present disclosure relates to the use of any one of the foregoing viruses as a vaccine vector. In particular, the present technology relates to the use of MVAΔE5R or MVAΔE5R-hFlt3L-OX40L as vaccine vectors for cancer vaccines. In some embodiments, the present technology relates to a recombinant poxvirus selected from MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-CTLA-4, and MYXVΔM62RΔM63RΔM64RΔM31R-hFlt3L-OX40L-IL-12-IL-15/IL-15Rα-CTLA-4, or combinations thereof, alone or in combination with immune checkpoint blocking agents, immunomodulatory agents, and/or anti-cancer drugs as an immunotherapeutic and/or oncolytic composition.

### BACKGROUND

The following description is provided to assist the understanding of the reader. None of the information provided or references cited is admitted to be prior art.

Malignant tumors such as melanoma are inherently resistant to conventional therapies and present significant therapeutic challenges. Immunotherapy is an evolving area of research and an additional option for the treatment of certain types of cancers. The immunotherapy approach rests on the rationale that the immune system may be stimulated to identify tumor cells and target them for destruction. Despite presentation of antigens by cancer cells and the presence of immune cells that could potentially react against tumor cells, in many cases, the immune system is not activated or is affirmatively suppressed. Key to this phenomenon is the ability of tumors to protect themselves from immune response by coercing cells of the immune system to inhibit other cells of the immune system. Tumors develop a number of immunomodulatory mechanisms to evade antitumor immune responses.

Thus, improved immunotherapeutic approaches are needed to enhance host antitumor immunity and target tumor cells for destruction.

### SUMMARY

In one aspect, the present disclosure provides a recombinant modified vaccinia Ankara (MVA) virus comprising a mutant C7 gene and a heterologous nucleic acid molecule encoding OX40L (MVAΔC7L-OX40L). In some embodiments, the recombinant MVAΔC7L-OX40L virus further comprises a heterologous nucleic acid encoding human Fms-like tyrosine kinase 3 ligand (hFlt3L) (MVAΔC7L-hFlt3L-OX40L). In some embodiments, the recombinant MVAΔC7L-OX40L virus of the present technology further comprises a mutant thymidine kinase (TK) gene. In some embodiments, the recombinant MVAΔC7L-OX40L virus comprising the mutant TK gene comprises replacement of at least a portion of the gene with one or more gene cassettes comprising a heterologous nucleic acid molecule. In some embodiments, the one or more gene cassettes comprise the heterologous nucleic acid molecule encoding OX40L. In some embodiments, the mutant C7 gene comprises an insertion of one or more gene cassettes comprising a heterologous nucleic acid molecule. In some embodiments, the mutant C7 gene comprises replacement of all or at least a portion of the gene with one or more gene cassettes comprising a heterologous nucleic acid molecule. In some embodiments, the one or more gene cassettes comprise a heterologous nucleic acid molecule encoding hFlt3L. In some embodiments, the mutant C7 gene comprises replacement of at least a portion of the gene with one or more gene cassettes comprising the heterologous nucleic acid molecule encoding hFlt3L, and wherein the virus further comprises a mutant TK gene comprising replacement of at least a portion of the TK gene with one or more gene cassettes comprising the heterologous nucleic acid molecule encoding OX40L (MVAΔC7L-hFlt3L-TK(−)-OX40L). In some embodiments, the OX40L is expressed from within a MVA viral gene. In some embodiments, the OX40L is expressed from within a viral gene selected from the group consisting of the thymidine kinase (TK) gene, the C7 gene, the C11 gene, the K3 gene, the F1 gene, the F2 gene, the F4 gene, the F6 gene, the F8 gene, the F9 gene, the F11 gene, the F14.5 gene, the J2 gene, the A46 gene, the E3L gene, the B18R gene (WR200), the E5R gene, the K7R gene, the C12L gene, the B8R gene, the B14R gene, the N1L gene, the K1L gene, the C16 gene, the M1L gene, the N2L gene, and the WR199 gene. In some embodiments, the OX40L is expressed from within the TK gene. In some embodiments, the OX40L is expressed from within the TK gene and the hFlt3L is expressed from within the C7 gene. In some embodiments, the virus further comprises a heterologous nucleic acid molecule encoding one or more of hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or a deletion of any one or more of E3L (ΔE3L), E3LΔ83N, B2R (ΔB2R), B19R (B18R; ΔWR200), IL18BP, E5R, K7R, C12L, B8R, B14R, N1L, C11R, K1L, M1L, N2L, or WR199. In some embodiments, the recombinant MVA virus exhibits one or more of the following characteristics: induction of increased levels of effector T-cells in tumor cells as compared to tumor cells infected with the corresponding MVAΔC7L virus; induction of increased splenic production of effector T-cells as compared to the corresponding MVAΔC7L virus; and reduction of tumor volume in tumor cells contacted with the recombinant MVAΔC7L-OX40L virus as compared to tumor cells contacted with the corresponding MVAΔC7L virus. In some embodiments, the tumor cells comprise melanoma cells.

In another aspect, the present disclosure provides, an immunogenic composition comprising the recombinant MVAΔC7L-OX40L virus of the present technology. In some embodiments, the immunogenic composition further comprises a pharmaceutically acceptable carrier. In some embodiments, the immunogenic composition of the present technology comprises a pharmaceutically acceptable adjuvant.

In another aspect, the present disclosure provides a method for treating a solid tumor in a subject in need thereof, the method comprising delivering to a tumor a composition comprising an effective amount of the recombinant MVAΔC7L-the present technology. In some embodiments, the treatment comprises one or more of the following: inducing an immune response in the subject against the tumor or enhancing or promoting an ongoing immune response against the tumor in the subject, reducing the size of the tumor, eradicating the tumor, inhibiting the growth of the tumor, inhibiting metastatic growth of the tumor, inducing apoptosis of tumor cells, or prolonging survival of the subject. In some embodiments, the method for treating a solid tumor in a subject in need thereof comprises the induction, enhancement, or promotion of the immune response comprises one or more of the following: increased levels of effector T-cells in tumor cells as compared to tumor cells infected with the corresponding MVAΔC7L virus; and increased splenic production of effector T-cells as compared to the corresponding MVAΔC7L virus. In some embodiments, the method of treating a solid tumor in a subject in need thereof the composition is administered by intratumoral or intravenous injection or a simultaneous or sequential combination of intratumoral and intravenous injection. In some embodiments of the method of treating a solid tumor in a subject in need thereof, the tumor is melanoma, colon, breast, bladder, or prostate carcinoma. In some embodiments of the method of treating a solid tumor in a subject in need thereof, the composition comprises one or more immune checkpoint blocking agents. In some embodiments of the method of treating a solid tumor in a subject in need thereof, the method further comprises administering to the subject one or more immune checkpoint blocking agents. In some embodiments of the method of treating a solid tumor in a subject in need thereof, the one or more immune checkpoint blocking agent is selected from the group consisting of anti-PD-1 antibody, anti-PD-L1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof. In some embodiments of the method of treating a solid tumor in a subject in need thereof, the one or more immune checkpoint blocking agents comprises anti-PD-1 antibody. In some embodiments of the method of treating a solid tumor in a subject in need thereof, the one or more immune checkpoint blocking agents comprises anti-PD-L1 antibody. In some embodiments of the method of treating a solid tumor in a subject in need thereof, the one or more immune checkpoint blocking agents comprises anti-CTLA-4 antibody. In some embodiments of the method of treating a solid tumor in a subject in need thereof, the combination of the MVAΔC7L-OX40L or MVAΔC7L-hFlt3L-OX40L and the immune checkpoint blocking agent has a synergistic effect in the treatment of the tumor as compared to administration of either the MVAΔC7L-OX40L or MVAΔC7L-hFlt3L-OX40L or of the immune checkpoint blocking agent alone.

In another aspect, the present disclosure provides a method of stimulating an immune response comprising administering to a subject an effective amount of the virus of the present technology (e.g., MVAΔC7L-OX40L, MVAΔC7L-hFlt3L-OX40L) or an immunogenic composition of the present technology. In some embodiments, the method further comprises administering to the subject one or more immune checkpoint blocking agents. In some embodiments, the one or more immune checkpoint blocking agents is selected from the group consisting of anti-PD-1 antibody, anti-PD-L1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof. In some embodiments, the one or more immune checkpoint blocking agents comprises anti-PD-1 antibody. In some embodiments, the one or more immune checkpoint blocking agents comprises anti-PD-L1 antibody. In some embodiments, the one or more immune checkpoint blocking agents comprises anti-CTLA-4 antibody.

In another aspect, the present disclosure provides, a recombinant modified vaccinia Ankara (MVA) virus comprising a mutant E3 gene and a heterologous nucleic acid molecule encoding OX40L (MVAΔE3L-OX40L). In some embodiments, the recombinant MVAΔE3L-OX40L virus comprises a mutant thymidine kinase (TK) gene. In some embodiments, the mutant TK gene comprises replacement of at least a portion of the gene with one or more gene cassettes comprising a heterologous nucleic acid molecule. In some embodiments, the one or more gene cassettes comprise the heterologous nucleic acid molecule encoding OX40L. In some embodiments of the virus of the present technology, the OX40L is expressed from within a MVA viral gene. In some embodiments of the virus of the present technology, the OX40L is expressed from within a viral gene selected from the group consisting of the thymidine kinase (TK) gene, the C7 gene, the C11 gene, the K3 gene, the F1 gene, the F2 gene, the F4 gene, the F6 gene, the F8 gene, the F9 gene, the F11 gene, the F14.5 gene, the J2 gene, the A46 gene, the E3L gene, the B18R gene (WR200), the E5R gene, the K7R gene, the C12L gene, the B8R gene, the B14R gene, the N1L gene, the K1L gene, the C16 gene, the M1L gene, the N2L gene, and the WR199 gene. In some embodiments, of the virus of the present technology, the OX40L is expressed from within the TK gene. In some embodiments, of the virus of the present technology, the virus comprises a heterologous nucleic acid molecule encoding one or more of hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or a deletion of any one or more of E3LΔ83N, B2R (ΔB2R), B19R (B18R; ΔWR200), E5R, K7R, C12L (IL18BP), B8R, B14R, N1L, C11R, K1L, M1L, N2L, or WR199. In some embodiments, of the virus of the present technology, the recombinant MVA virus exhibits one or more of the following characteristics: induction of increased levels of effector T-cells in tumor cells as compared to tumor cells infected with the corresponding MVAΔE3L virus; induction of increased splenic production of effector T-cells as compared to the corresponding MVAΔE3L virus; and reduction of tumor volume in tumor cells contacted with the recombinant MVAΔE3L-OX40L virus as compared to tumor cells contacted with the corresponding MVAΔE3L virus. In some embodiments, of the virus of the present technology, the tumor cells comprise melanoma cells. In some embodiments, the mutant E3 gene is at least partially deleted, is not expressed, is expressed at levels so low as to have no effect, or expressed as a non-functional protein. In some embodiments, the mutant TK gene is at least partially deleted, is not expressed, is expressed at levels so low as to have no effect, or expressed as a non-functional protein.

In another aspect, the present disclosure provides an immunogenic composition comprising the recombinant MVAΔE3L-OX40L virus of the present technology. In some embodiments, the immunogenic composition of the present technology comprises a pharmaceutically acceptable carrier. In some embodiments, the immunogenic composition of the present technology comprises a pharmaceutically acceptable adjuvant.

In another aspect, the present disclosure provided a method for treating a solid tumor in a subject in need thereof, the method comprising delivering to a tumor a composition comprising an effective amount of the recombinant MVAΔE3L-OX40L virus of the present technology or an immunogenic composition of the present technology. In some embodiments of the method for treating a solid tumor in a subject in need thereof, the treatment comprises one or more of the following: inducing an immune response in the subject against the tumor or enhancing or promoting an ongoing immune response against the tumor in the subject, reducing the size of the tumor, eradicating the tumor, inhibiting the growth of the tumor, inhibiting metastatic growth of the tumor, inducing apoptosis of tumor cells, or prolonging survival of the subject. In some embodiments of the method for treating a solid tumor in a subject in need thereof, the induction, enhancement, or promotion of the immune response comprises one or more of the following: increased levels of effector T-cells in tumor cells as compared to tumor cells infected with the corresponding MVAΔE3L virus; and increased splenic production of effector T-cells as compared to the corresponding MVAΔE3L virus. In some embodiments of the method for treating a solid tumor in a subject in need thereof, the composition is administered by intratumoral or intravenous injection or a simultaneous or sequential combination of intratumoral and intravenous injection. In some embodiments of the method for treating a solid tumor in a subject in need thereof, the tumor is melanoma, colon, breast, bladder, or prostate carcinoma. In some embodiments of the method for treating a solid tumor in a subject in need thereof, the composition further comprises one or more immune checkpoint blocking agents. In some embodiments of the method for treating a solid tumor in a subject in need thereof, the one or more immune checkpoint blocking agent is selected from the group consisting of anti-PD-1 antibody, anti-PD-L1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof. In some embodiments of the method for treating a solid tumor in a subject in need thereof, the one or more immune checkpoint blocking agents comprises anti-PD-1 antibody. In some embodiments of the method for treating a solid tumor in a subject in need thereof, the one or more immune checkpoint blocking agents comprises anti-PD-L1 antibody. In some embodiments of the method for treating a solid tumor in a subject in need thereof, the one or more immune checkpoint blocking agents comprises anti-CTLA-4 antibody. In some embodiments of the method for treating a solid tumor in a subject in need thereof, the combination of the MVAΔE3L-OX40L and the immune checkpoint blocking agent has a synergistic effect in the treatment of the tumor as compared to administration of either MVAΔE3L-OX40L or the immune checkpoint blocking agent alone.

In another aspect, the present disclosure provides a method of stimulating an immune response comprising administering to a subject an effective amount of a virus of the present technology (e.g., MVAΔE3L-OX40L) or an immunogenic composition of the present technology. In some embodiments, the method further comprises administering one or more immune checkpoint blocking agents to the subject. In some embodiments, the one or more immune checkpoint blocking agents is selected from the group consisting of anti-PD-1 antibody, anti-PD-L1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof. In some embodiments, the one or more immune checkpoint blocking agents comprises anti-PD-1 antibody. In some embodiments, the one or more immune checkpoint blocking agents comprises anti-PD-L1 antibody. In some embodiments, the one or more immune checkpoint blocking agents comprises anti-CTLA-4 antibody. In some embodiments, the combination of MVAΔE3L-OX40L and the immune checkpoint blocking agent has a synergistic effect in the treatment of the tumor as compared to administration of either MVAΔE3L-OX40L or the immune checkpoint blocking agent alone.

In another aspect, the present disclosure provides a recombinant vaccinia virus (VACV) comprising a mutant C7 gene and a heterologous nucleic acid molecule encoding OX40L (VACVΔC7L-OX40L). In some embodiments, the virus comprises a heterologous nucleic acid encoding human Fms-like tyrosine kinase 3 ligand (hFlt3L) (VACVΔC7L-hFlt3L-OX40L). In some embodiments, the virus comprises a mutant thymidine kinase (TK) gene. In some embodiments, the mutant TK gene comprises replacement of at least a portion of the gene with one or more gene cassettes comprising a heterologous nucleic acid molecule.

In some embodiments, the one or more gene cassettes comprise the heterologous nucleic acid molecule encoding OX40L. In some embodiments, the mutant C7 gene comprises an insertion of one or more gene cassettes comprising a heterologous nucleic acid molecule. In some embodiments, the mutant C7 gene comprises replacement of all or at least a portion of the gene with one or more gene cassettes comprising a heterologous nucleic acid molecule.

In some embodiments, the one or more gene cassettes comprise a heterologous nucleic acid molecule encoding hFlt3L. In some embodiments, the mutant C7 gene comprises replacement of at least a portion of the gene with one or more gene cassettes comprising the heterologous nucleic acid molecule encoding hFlt3L, and wherein the virus further comprises a mutant TK gene comprising replacement of at least a portion of the TK gene with one or more gene cassettes comprising the heterologous nucleic acid molecule encoding OX40L (VACVΔC7L-hFlt3L-TK(−)-OX40L). In some embodiments, the OX40L is expressed from within a vaccinia viral gene. In some embodiments, the OX40L is expressed from within a viral gene selected from the group consisting of the thymidine kinase (TK) gene, the C7 gene, the C11 gene, the K3 gene, the F1 gene, the F2 gene, the F4 gene, the F6 gene, the F8 gene, the F9 gene, the F11 gene, the F14.5 gene, the J2 gene, the A46 gene, the E3L gene, the B18R gene (WR200), the E5R gene, the K7R gene, the C12L gene, the B8R gene, the B14R gene, the N1L gene, the K1L gene, the C16 gene, the M1L gene, the N2L gene, and the WR199 gene. In some embodiments, the OX40L is expressed from within the TK gene. In some embodiments, the OX40L is expressed from within the TK gene and the hFlt3L is expressed from within the C7 gene. In some embodiments, the virus further comprises a heterologous nucleic acid molecule encoding one or more of hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or a deletion of any one or more of E3L (ΔE3L), E3LΔ83N, B2R (ΔB2R), B19R (B18R; ΔWR200), E5R, K7R, C12L (IL18BP), B8R, B14R, N1L, C11R, K1L, M1L, N2L, or WR199. In some embodiments, the virus further comprises a heterologous nucleic acid encoding hIL-12 and a heterologous nucleic acid encoding anti-huCTLA-4 (VACVΔC7L-anti-huCTLA-4-hFlt3L-OX40L-hIL-12). In some embodiments, the recombinant VACVΔC7L-OX40L virus exhibits one or more of the following characteristics: induction of increased levels of effector T-cells in tumor cells as compared to tumor cells infected with the corresponding VACVΔC7L virus; induction of increased splenic production of effector T-cells as compared to the corresponding VACVΔC7L virus; and reduction of tumor volume in tumor cells contacted with the recombinant VACVΔC7L-OX40L virus as compared to tumor cells contacted with the corresponding VACVΔC7L virus. In some embodiments, the tumor cells comprise melanoma cells.

In another aspect, the present disclosure provides, an immunogenic composition comprising the recombinant VACVΔC7L-OX40L virus of the present technology. In some embodiments, the immunogenic composition comprises a pharmaceutically acceptable carrier. In some embodiments, the immunogenic composition comprises a pharmaceutically acceptable adjuvant.

In another aspect, the present disclosure provides a method for treating a solid tumor in a subject in need thereof, the method comprising delivering to a tumor a composition comprising an effective amount of the recombinant VACVΔC7L-OX40L virus of the present technology or the immunogenic composition of the present technology. In some embodiments of the method for treating a solid tumor in a subject in need thereof, the treatment comprises one or more of the following: inducing an immune response in the subject against the tumor or enhancing or promoting an ongoing immune response against the tumor in the subject, reducing the size of the tumor, eradicating the tumor, inhibiting the growth of the tumor, inhibiting metastatic growth of the tumor, inducing apoptosis of tumor cells, or prolonging survival of the subject. In some embodiments of the method for treating a solid tumor in a subject in need thereof, the treatment comprises the induction, enhancement, or promotion of the immune response comprises one or more of the following: increased levels of effector T-cells in tumor cells as compared to tumor cells infected with the corresponding VACVΔC7L virus; and increased splenic production of effector T-cells as compared to the corresponding VACVΔC7L virus. In some embodiments of the method for treating a solid tumor in a subject in need thereof, the composition is administered by intratumoral or intravenous injection or a simultaneous or sequential combination of intratumoral and intravenous injection. In some embodiments of the method for treating a solid tumor in a subject in need thereof, the tumor is melanoma, colon, breast, bladder, or prostate carcinoma. In some embodiments of the method for treating a solid tumor in a subject in need thereof, the composition further comprises one or more immune checkpoint blocking agents. In some embodiments, the method further comprises administering to the subject one or more immune checkpoint blocking agents. In some embodiments the one or more immune checkpoint blocking agents is selected from the group consisting of anti-PD-1 antibody, anti-PD-L1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof. In some embodiments of the method for treating a solid tumor in a subject in need thereof, the one or more immune checkpoint blocking agents comprises the one or more immune checkpoint blocking agent comprises anti-PD-1 antibody. In some embodiments of the method for treating a solid tumor in a subject in need thereof, the one or more immune checkpoint blocking agents comprises the one or more immune checkpoint blocking agent comprises anti-PD-L1 antibody. In some embodiments of the method for treating a solid tumor in a subject in need thereof, comprises the one or more immune checkpoint blocking agent comprises anti-CTLA-4 antibody. In some embodiments of the method for treating a solid tumor in a subject in need thereof, the combination of the VACVΔC7L-OX40L or VACVΔC7L-hFlt3L-OX40L and the immune checkpoint blocking agent has a synergistic effect in the treatment of the tumor as compared to administration of VACVΔC7L-OX40L or VACVΔC7L-hFlt3L-OX40L or of the immune checkpoint blocking agent alone.

In another aspect, the present disclosure provides, a method for stimulating an immune response comprising administering to a subject an effective amount of the virus of the present technology (e.g., VACVΔC7L-OX40L or VACVΔC7L-hFlt3L-OX40L) or an immunogenic composition of the present technology. In some embodiments, the method further comprises administering one or more immune checkpoint blocking agents. In some embodiments, the one or more immune checkpoint blocking agents is selected from the group consisting of anti-PD-1 antibody, anti-PD-L1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof. In some embodiments, the immune checkpoint blocking agent comprises anti-PD-1 antibody. In some embodiments, the immune checkpoint blocking agent comprises anti-PD-L1 antibody. In some embodiments, the immune checkpoint blocking agent comprises anti-CTLA-4 antibody.

In another aspect, the present disclosure provides, a recombinant modified vaccinia Ankara (MVA) virus nucleic acid sequence, wherein the nucleic acid sequence between position 75,560 and 76,093 of SEQ ID NO: 1 is replaced with a heterologous nucleic acid sequence comprising an open reading frame that encodes OX40L, and wherein the MVA further comprises a C7 mutant. In some embodiments of the MVA virus of the present technology, the nucleic acid sequence between position 18,407 and 18,859 of SEQ ID NO: 1 is replaced with a heterologous nucleic acid sequence comprising an open reading frame that encodes human Fms-like tyrosine kinase 3 ligand (hFlt3L).

In another aspect, the present disclosure provides a recombinant modified vaccinia Ankara (MVA) virus nucleic acid sequence, wherein the nucleic acid sequence between position 75,798 to 75,868 of SEQ ID NO: 1 is replaced with a heterologous nucleic acid sequence comprising an open reading frame that encodes OX40L or encodes human Fms-like tyrosine kinase 3 ligand (hFlt3L), and wherein the MVA further comprises an E3 mutant.

In another aspect, the present disclosure provides a recombinant vaccinia virus (VACV) nucleic acid sequence, wherein the nucleic acid sequence between position 80,962 and 81,032 of SEQ ID NO: 2 is replaced with a heterologous nucleic acid sequence comprising an open reading frame that encodes OX40L or, and wherein the VACV further comprises a C7 mutant. In some embodiments the recombinant VACV of the present technology the nucleic acid sequence between position 15,716 and 16,168 of SEQ ID NO: 2 is replaced with a heterologous nucleic acid sequence comprising an open reading frame that encodes human Fms-like tyrosine kinase 3 ligand (hFlt3L).

In another aspect, the present disclosure provides a nucleic acid sequence encoding the recombinant MVAΔC7L-OX40L virus of the present technology.

In another aspect, the present disclosure provides a nucleic acid sequence encoding the recombinant MVAΔE3L-OX40L virus of the present technology.

In another aspect, the present disclosure provides a nucleic acid sequence encoding the recombinant VACVΔC7L-OX40L virus of any one of the present technology.

In another aspect, the present disclosure provides a kit comprising the recombinant MVAΔC7L-OX40L virus of the present technology or the immunogenic composition of the present technology, and instructions for use.

In another aspect, the present disclosure provides a kit comprising the recombinant MVAΔE3L-OX40L virus of any one of the present technology or the immunogenic composition of the present technology, and instructions for use.

In another aspect, the present disclosure provides a kit comprising the recombinant VACVΔC7L-OX40L virus of the present technology or the immunogenic composition of the present technology, and instructions for use.

In some embodiments, the present disclosure provides a recombinant MVAΔC7L-OX40L virus wherein the mutant C7 gene is at least partially deleted, is not expressed, is expressed at levels so low as to have no effect, or expressed as a non-functional protein. In some embodiments, the present disclosure provides a recombinant MVAΔC7L-OX40L virus, wherein the mutant TK gene is at least partially deleted, is not expressed, is expressed at levels so low as to have no effect, or expressed as a non-functional protein. In some embodiments, the present disclosure provides a recombinant MVAΔE3L-OX40L virus wherein the mutant E3 gene is at least partially deleted, is not expressed, is expressed at levels so low as to have no effect, or expressed as a non-functional protein. In some embodiments, the present disclosure provides a recombinant MVAΔE3L-OX40L virus wherein the mutant TK gene is at least partially deleted, is not expressed, is expressed at levels so low as to have no effect, or expressed as a non-functional protein. In some embodiments, the present technology provides a recombinant VACVΔC7L-OX40L virus wherein the mutant C7 gene is at least partially deleted, is not expressed, is expressed at levels so low as to have no effect, or expressed as a non-functional protein. In some embodiments, the present disclosure provides a recombinant VACVΔC7L-OX40L virus wherein the mutant TK gene is at least partially deleted, is not expressed, is expressed at levels so low as to have no effect, or expressed as a non-functional protein.

In another aspect, the present disclosure provides a method for treating a solid tumor in a subject in need thereof, the method comprising administering to the subject an antigen and a therapeutically effective amount of an adjuvant comprising a recombinant modified vaccinia Ankara (MVA) virus comprising a mutant C7 gene and a heterologous nucleic acid encoding OX40L (MVAΔC7L-OX40L). In some embodiments, the MVAΔC7L-OX40L virus further comprises a heterologous nucleic acid encoding human Fms-like tyrosine kinase 3 ligand (hFlt3L) (MVAΔC7L-hFlt3L-OX40L). In some embodiments, the virus further comprises a mutant thymidine kinase (TK) gene. In some embodiments, the mutant TK gene comprises replacement of at least a portion of the gene with one or more gene cassettes comprising a heterologous nucleic acid molecule. In some embodiments, the one or more gene cassettes comprise the heterologous nucleic acid molecule encoding OX40L. In some embodiments, the mutant C7 gene comprises an insertion of one or more gene cassettes comprising a heterologous nucleic acid molecule. In some embodiments, the mutant C7 gene comprises replacement of all or at least a portion of the gene with one or more gene cassettes comprising a heterologous nucleic acid. In some embodiments, the one or more gene cassettes comprise a heterologous nucleic acid molecule encoding hFlt3L. In some embodiments, the mutant C7 gene comprises replacement of at least a portion of the gene with one or more gene cassettes comprising the heterologous nucleic acid molecule encoding hFlt3L, and wherein the virus further comprises a mutant TK gene comprising replacement of at least a portion of the TK gene with one or more gene cassettes comprising the heterologous nucleic acid molecule encoding OX40L (MVAΔC7L-hFlt3L-TK(−)-OX40L). In some embodiments, the OX40L is expressed from within a MVA viral gene. In some embodiments, the OX40L is expressed from within a viral gene selected from the group consisting of the thymidine kinase (TK) gene, the C7 gene, the C11 gene, the K3 gene, the F1 gene, the F2 gene, the F4 gene, the F6 gene, the F8 gene, the F9 gene, the F11 gene, the F14.5 gene, the J2 gene, the A46 gene, the E3L gene, the B18R gene (WR200), the E5R gene, the K7R gene, the C12L gene, the B8R gene, the B14R gene, the N1L gene, the K1L gene, the C16 gene, the M1L gene, the N2L gene, and the WR199 gene. In some embodiments, the OX40L is expressed from within the TK gene. In some embodiments, the OX40L is expressed from within the TK gene and the hFlt3L is expressed from within the C7 gene. In some embodiments, the virus further comprises a heterologous nucleic acid molecule encoding one or more of hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or a deletion of any one or more of E3L (ΔE3L), E3LΔ83N, B2R (ΔB2R), B19R (B18R; ΔWR200), IL18BP, E5R, K7R, C12L, B8R, B14R, N1L, C11R, K1L, M1L, N2L, or WR199.

In some embodiments, the antigen is selected from the group consisting of tumor differentiation antigens, cancer testis antigens, neoantigens, viral antigens in the case of tumors associated with oncogenic virus infection, GPA33, HER2/neu, GD2, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, tyrosinase-related proteins 1 and 2, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, p53, kras, lung resistance protein (LRP) Bcl-2, prostate specific antigen (PSA), Ki-67, CEACAM6, colon-specific antigen-p (CSAp), NY-ESO-1, human papilloma virus E6 and E7, and any combination thereof.

In some embodiments, the administration step comprises administering the antigen and adjuvant in one or more doses and/or wherein the antigen and adjuvant are administered separately, sequentially, or simultaneously.

In some embodiments, the method further comprises administering to the subject an immune checkpoint blockade agent selected from the group consisting of anti-PD-1 antibody, anti-PD-L1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof.

In some embodiments, the antigen and adjuvant are delivered to the subject separately, sequentially, or simultaneously with the administration of the immune checkpoint blockade agent.

In some embodiments, the treatment comprises one or more of the following: inducing an immune response in the subject against the tumor or enhancing or promoting an ongoing immune response against the tumor in the subject, reducing the size of the tumor, eradicating the tumor, inhibiting growth of the tumor, inhibiting metastatic growth of the tumor, inducing apoptosis of the tumor cells, or prolonging survival of the subject. In some embodiments, the induction, enhancement, or promotion of the immune response comprises one or more of the following: (i) increased levels of interferon gamma (IFN-γ) expression in T-cells in the spleen, draining lymph nodes, and/or serum as compared to an untreated control sample; (ii) increased levels of antigen-specific T-cells in the spleen, draining lymph nodes, and/or serum as compared to an untreated control sample; and (iii) increased levels of antigen-specific immunoglobulin in serum as compared to an untreated control sample. In some embodiments, the antigen-specific immunoglobulin is IgG1 or IgG2.

In some embodiments, the antigen and adjuvant are formulated to be administered intratumorally, intramuscularly, intradermally, or subcutaneously.

In some embodiments, the tumor is selected from the group consisting of melanoma, colorectal cancer, breast cancer, bladder cancer, prostate cancer, lung cancer, pancreatic cancer, ovarian cancer, squamous cell carcinoma of the skin, Merkel cell carcinoma, gastric cancer, liver cancer, and sarcoma.

In some embodiments, the MVAΔC7L-OX40L virus is administered at a dosage per administration of about 10⁵ to about 10¹⁰ plaque-forming units (pfu).

In some embodiments of the method, the subject is human.

In one aspect, the present disclosure provides an immunogenic composition comprising the antigen and the adjuvant of the present technology. In some embodiments, the immunogenic composition further comprises a pharmaceutically acceptable carrier. In some embodiments, the antigen is selected from the group consisting of tumor differentiation antigens, cancer testis antigens, neoantigens, viral antigens in the case of tumors associated with oncogenic virus infection, GPA33, HER2/neu, GD2, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, tyrosinase-related proteins 1 and 2, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, p53, kras, lung resistance protein (LRP) Bcl-2, prostate specific antigen (PSA), Ki-67, CEACAM6, colon-specific antigen-p (CSAp), NY-ESO-1, human papilloma virus E6 and E7, and any combination thereof. In some embodiments, the immunogenic composition further comprises an immune checkpoint blockade agent selected from the group consisting of anti-PD-1 antibody, anti-PD-L1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof.

In one aspect, the present disclosure provides a kit comprising instructions for use, a container means, and a separate portion of each of: (a) an antigen; and (b) an adjuvant of the present technology. In some embodiments of the kit, the antigen is selected from the group consisting of tumor differentiation antigens, cancer testis antigens, neoantigens, viral antigens in the case of tumors associated with oncogenic virus infection, GPA33, HER2/neu, GD2, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, tyrosinase-related proteins 1 and 2, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, p53, kras, lung resistance protein (LRP) Bcl-2, prostate specific antigen (PSA), Ki-67, CEACAM6, colon-specific antigen-p (CSAp), NY-ESO-1, human papilloma virus E6 and E7, and any combination thereof.

In some embodiments, the kit further comprises (c) an immune checkpoint blockade agent selected from the group consisting of anti-PD-1 antibody, anti-PD-L1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof.

In some embodiments of the methods of the present technology, the antigen is a neoantigen selected from the group consisting of M27 (REGVELCPGNKYEMRRHGTTHSLVIHD) (SEQ ID NO: 17), M30 (PSKPSFQEFVDWENVSPELNSTDQPFL) (SEQ ID NO: 18), M48 (SHCHWNDLAVIPAGVVHNWDFEPRKVS) (SEQ ID NO: 19), and combinations thereof.

In some embodiments of the immunogenic compositions of the present technology, the antigen is a neoantigen selected from the group consisting of M27 (REGVELCPGNKYEMRRHGTTHSLVIHD) (SEQ ID NO: 17), M30 (PSKPSFQEFVDWENVSPELNSTDQPFL) (SEQ ID NO: 18), M48 (SHCHWNDLAVIPAGVVHNWDFEPRKVS) (SEQ ID NO: 19), and combinations thereof.

In some embodiments of the kit of the present technology, the antigen is a neoantigen selected from the group consisting of M27 (REGVELCPGNKYEMRRHGTTHSLVIHD) (SEQ ID NO: 17), M30 (PSKPSFQEFVDWENVSPELNSTDQPFL) (SEQ ID NO: 18), M48 (SHCHWNDLAVIPAGVVHNWDFEPRKVS) (SEQ ID NO: 19), and combinations thereof.

In one aspect, the present disclosure provides a modified vaccinia Ankara (MVA) virus genetically engineered to comprise a mutant E5R gene (MVAΔE5R). In some embodiments, the virus further comprises a heterologous nucleic acid molecule encoding one or more of OX40L, hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or a deletion of any one or more of thymidine kinase (TK), C7 (ΔC7L), E3L (ΔE3L), E3LΔ83N, B2R (ΔB2R), B19R (B18R; ΔWR200), IL18BP, K7R, C12L, B8R, B14R, N1L, C11R, K1L, M1L, N2L, or WR199. In some embodiments, the mutant E5R gene comprises replacement of at least a portion of the gene with one or more gene cassettes comprising the heterologous nucleic acid molecule. In some embodiments, the one or more gene cassettes comprises a heterologous nucleic acid molecule encoding OX40L (MVAΔE5R-OX40L). In some embodiments, the one or more gene cassettes further comprises a heterologous nucleic acid molecule encoding human Fms-like tyrosine kinase 3 ligand (hFlt3L) (MVAΔE5R-OX40L-hFlt3L). In some embodiments, the one or more gene cassettes comprises a heterologous nucleic acid molecule encoding human Fms-like tyrosine kinase 3 ligand (hFlt3L) (MVAΔE5R-hFlt3L). In some embodiments, the heterologous nucleic acid is expressed from within a viral gene selected from the group consisting of the thymidine kinase (TK) gene, the C7 gene, the C11 gene, the K3 gene, the F1 gene, the F2 gene, the F4 gene, the F6 gene, the F8 gene, the F9 gene, the F11 gene, the F14.5 gene, the J2 gene, the A46 gene, the E3L gene, the B18R gene (WR200), the E5R gene, the K7R gene, the C12L gene, the B8R gene, the B14R gene, the N1L gene, the K1L gene, the C16 gene, the M1L gene, the N2L gene, and the WR199 gene. In some embodiments, the virus further comprises a mutant thymidine kinase (TK) gene. In some embodiments, the mutant TK gene comprises replacement of at least a portion of the gene with one or more gene cassettes comprising a heterologous nucleic acid molecule. In some embodiments, the virus further comprises a mutant C7 gene. In some embodiments, the mutant C7 gene comprises an insertion of one or more gene cassettes comprising a heterologous nucleic acid molecule. In some embodiments, the mutant C7 gene comprises replacement of all or at least a portion of the gene with one or more gene cassettes comprising a heterologous nucleic acid molecule.

In one aspect, the present disclosure provides an immunogenic composition comprising the MVAΔE5R virus. In some embodiments, the immunogenic composition further comprises a pharmaceutically acceptable carrier. In some embodiments, the immunogenic composition further comprises a pharmaceutically acceptable adjuvant.

In one aspect, the present disclosure provides a method for treating a solid tumor in a subject in need thereof, the method comprising delivering to a tumor a composition comprising an effective amount of the MVAΔE5R virus or the immunogenic composition. In some embodiments, the treatment comprises one or more of the following: inducing an immune response in the subject against the tumor or enhancing or promoting an ongoing immune response against the tumor in the subject, reducing the size of the tumor, eradicating the tumor, inhibiting the growth of the tumor, inhibiting metastatic growth of the tumor, inducing apoptosis of tumor cells, or prolonging survival of the subject. In some embodiments, the composition is administered by intratumoral or intravenous injection or a simultaneous or sequential combination of intratumoral and intravenous injection. In some embodiments, the tumor is melanoma, colon, breast, bladder, or prostate carcinoma. In some embodiments, the composition further comprises one or more agents selected from: one or more immune checkpoint blocking agents; one or more anti-cancer drugs; fingolimod (FTY720); and any combination thereof.

In some embodiments, the method further comprises separately, sequentially, or simultaneously administering to the subject one or more agents selected from: one or more immune checkpoint blocking agents; one or more anti-cancer drugs, fingolimod (FTY720); and any combination thereof. In some embodiments, the one or more immune checkpoint blocking agents is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof; and/or the one or more anti-cancer drugs is selected from the group consisting of a Mek inhibitor (U0126, selumitinib (AZD6244), PD98059, trametinib, cobimetinib), an EGFR inhibitor (lapatinib (LPN), erlotinib (ERL)), a HER2 inhibitor (lapatinib (LPN), Trastuzumab), a Raf inhibitor (sorafenib (SFN)), a BRAF inhibitor (dabrafenib, vemurafenib), an anti-OX40 antibody, a GITR agonist antibody, an anti-CSFR antibody, a CSFR inhibitor, paclitaxel, TLR9 agonist CpG, and a VEGF inhibitor (Bevacizumab), and any combination thereof. In some embodiments, the one or more immune checkpoint blocking agents comprises anti-PD-L1 antibody. In some embodiments, the one or more immune checkpoint blocking agents comprises anti-PD-1 antibody. In some embodiments, the one or more immune checkpoint blocking agents comprises anti-CTLA-4 antibody. In some embodiments, the combination of the MVAΔE5R virus with the immune checkpoint blocking agent, anti-cancer drug, and/or fingolimod (FTY720) has a synergistic effect in the treatment of the tumor as compared to administration of either the MVAΔE5R virus or of the immune checkpoint blocking agent, anti-cancer drug, or fingolimod (FTY720) alone.

In one aspect, the present disclosure provides a method of stimulating an immune response comprising administering to a subject an effective amount of the virus or the immunogenic composition. In some embodiments, the method further comprises separately, sequentially, or simultaneously administering to the subject one or more agents selected from: one or more immune checkpoint blocking agents; one or more anti-cancer drugs; fingolimod (FTY720); and any combination thereof. In some embodiments, the one or more immune checkpoint blocking agents is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof; and/or the one or more anti-cancer drugs is selected from the group consisting of a Mek inhibitor (U0126, selumitinib (AZD6244), PD98059, trametinib, cobimetinib), an EGFR inhibitor (lapatinib (LPN), erlotinib (ERL)), a HER2 inhibitor (lapatinib (LPN), Trastuzumab), a Raf inhibitor (sorafenib (SFN)), a BRAF inhibitor (dabrafenib, vemurafenib), an anti-OX40 antibody, a GITR agonist antibody, an anti-CSFR antibody, a CSFR inhibitor, paclitaxel, TLR9 agonist CpG, and a VEGF inhibitor (Bevacizumab), and any combination thereof. In some embodiments, the one or more immune checkpoint blocking agents comprises anti-PD-L1 antibody. In some embodiments, the one or more immune checkpoint blocking agents comprises anti-PD-1 antibody. In some embodiments, the one or more immune checkpoint blocking agents comprises anti-CTLA-4 antibody. In some embodiments, the combination of the MVAΔE5R virus with the immune checkpoint blocking agent, anti-cancer drug, and/or fingolimod (FTY720) has a synergistic effect in the stimulation of an immune response as compared to administration of the MVAΔE5R virus or of the immune checkpoint blocking agent, anti-cancer drug, or fingolimod (FTY720) alone.

In one aspect, the present disclosure provides a nucleic acid encoding the engineered MVAΔE5R viruses described herein.

In one aspect, the present disclosure provides a kit comprising the engineered MVAΔE5R viruses described herein, and instructions for use.

In one aspect, the present disclosure provides a vaccinia virus (VACV) genetically engineered to comprise a mutant E5R gene (VACVΔE5R). In some embodiments, the virus further comprises a heterologous nucleic acid molecule encoding one or more of OX40L, hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or a deletion of any one or more of thymidine kinase (TK), C7 (ΔC7L), E3L (ΔE3L), E3LΔ83N, B2R (ΔB2R), B19R (B18R; ΔWR200), IL18BP, K7R, C12L, B8R, B14R, N1L, C11R, K1L, M1L, N2L, or WR199. In some embodiments, the mutant E5R gene comprises replacement of at least a portion of the gene with one or more gene cassettes comprising the heterologous nucleic acid molecule. In some embodiments, the one or more gene cassettes comprises a heterologous nucleic acid molecule encoding OX40L (VACVΔE5R-OX40L). In some embodiments, the one or more gene cassettes further comprises a heterologous nucleic acid molecule encoding human Fms-like tyrosine kinase 3 ligand (hFlt3L) (VACVΔE5R-OX40L-hFlt3L). In some embodiments, the one or more gene cassettes comprises a heterologous nucleic acid molecule encoding human Fms-like tyrosine kinase 3 ligand (hFlt3L) (VACVΔE5R-hFlt3L). In some embodiments, the heterologous nucleic acid is expressed from within a viral gene selected from the group consisting of the thymidine kinase (TK) gene, the C7 gene, the C11 gene, the K3 gene, the F1 gene, the F2 gene, the F4 gene, the F6 gene, the F8 gene, the F9 gene, the F11 gene, the F14.5 gene, the J2 gene, the A46 gene, the E3L gene, the B18R gene (WR200), the E5R gene, the K7R gene, the C12L gene, the B8R gene, the B14R gene, the N1L gene, the K1L gene, the C16 gene, the M1L gene, the N2L gene, and the WR199 gene. In some embodiments, the virus further comprises a mutant thymidine kinase (TK) gene. In some embodiments, the mutant TK gene comprises replacement of at least a portion of the gene with one or more gene cassettes comprising a heterologous nucleic acid molecule. In some embodiments, the virus further comprises a mutant C7 gene. In some embodiments, the mutant C7 gene comprises an insertion of one or more gene cassettes comprising a heterologous nucleic acid molecule. In some embodiments, the mutant C7 gene comprises replacement of all or at least a portion of the gene with one or more gene cassettes comprising a heterologous nucleic acid molecule.

In one aspect, the present disclosure provides an immunogenic composition comprising the VACVΔE5R virus. In some embodiments, the immunogenic composition further comprises a pharmaceutically acceptable carrier. In some embodiments, the immunogenic composition further comprises a pharmaceutically acceptable adjuvant.

In one aspect, the present disclosure provides a method for treating a solid tumor in a subject in need thereof, the method comprising delivering to a tumor a composition comprising an effective amount of the VACVΔE5R virus or the immunogenic composition.

In some embodiments, the treatment comprises one or more of the following: inducing an immune response in the subject against the tumor or enhancing or promoting an ongoing immune response against the tumor in the subject, reducing the size of the tumor, eradicating the tumor, inhibiting the growth of the tumor, inhibiting metastatic growth of the tumor, inducing apoptosis of tumor cells, or prolonging survival of the subject. In some embodiments, the composition is administered by intratumoral or intravenous injection or a simultaneous or sequential combination of intratumoral and intravenous injection. In some embodiments, the tumor is melanoma, colon, breast, bladder, or prostate carcinoma. In some embodiments, the composition further comprises one or more agents selected from: one or more immune checkpoint blocking agents; one or more anti-cancer drugs; fingolimod (FTY720); and any combination thereof.

In some embodiments, the method further comprises separately, sequentially, or simultaneously administering to the subject one or more agents selected from: one or more immune checkpoint blocking agents; one or more anti-cancer drugs, fingolimod (FTY720); and any combination thereof. In some embodiments, the one or more immune checkpoint blocking agents is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof; and/or the one or more anti-cancer drugs is selected from the group consisting of a Mek inhibitor (U0126, selumitinib (AZD6244), PD98059, trametinib, cobimetinib), an EGFR inhibitor (lapatinib (LPN), erlotinib (ERL)), a HER2 inhibitor (lapatinib (LPN), Trastuzumab), a Raf inhibitor (sorafenib (SFN)), a BRAF inhibitor (dabrafenib, vemurafenib), an anti-OX40 antibody, a GITR agonist antibody, an anti-CSFR antibody, a CSFR inhibitor, paclitaxel, TLR9 agonist CpG, and a VEGF inhibitor (Bevacizumab), and any combination thereof. In some embodiments, the one or more immune checkpoint blocking agents comprises anti-PD-L1 antibody. In some embodiments, the one or more immune checkpoint blocking agents comprises anti-PD-1 antibody. In some embodiments, the one or more immune checkpoint blocking agents comprises anti-CTLA-4 antibody. In some embodiments, the combination of the VACVΔE5R virus with the immune checkpoint blocking agent, anti-cancer drug, and/or fingolimod (FTY720) has a synergistic effect in the treatment of the tumor as compared to administration of the VACVΔE5R virus or of the immune checkpoint blocking agent, anti-cancer drug, or fingolimod (FTY720) alone.

In one aspect, the present disclosure provides a method of stimulating an immune response comprising administering to a subject an effective amount of the virus or the immunogenic composition of. In some embodiments, the method further comprises separately, sequentially, or simultaneously administering to the subject one or more agents selected from: one or more immune checkpoint blocking agents; one or more anti-cancer drugs; fingolimod (FTY720); and any combination thereof. In some embodiments, the one or more immune checkpoint blocking agents is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof; and/or the one or more anti-cancer drugs is selected from the group consisting of a Mek inhibitor (U0126, selumitinib (AZD6244), PD98059, trametinib, cobimetinib), an EGFR inhibitor (lapatinib (LPN), erlotinib (ERL)), a HER2 inhibitor (lapatinib (LPN), Trastuzumab), a Raf inhibitor (sorafenib (SFN)), a BRAF inhibitor (dabrafenib, vemurafenib), an anti-OX40 antibody, a GITR agonist antibody, an anti-CSFR antibody, a CSFR inhibitor, paclitaxel, TLR9 agonist CpG, and a VEGF inhibitor (Bevacizumab), and any combination thereof. In some embodiments, the one or more immune checkpoint blocking agents comprises anti-PD-L1 antibody. In some embodiments, the one or more immune checkpoint blocking agents comprises anti-PD-1 antibody. In some embodiments, the one or more immune checkpoint blocking agents comprises anti-CTLA-4 antibody. In some embodiments, the combination of the VACVΔE5R virus with the immune checkpoint blocking agent, anti-cancer drug, and/or fingolimod (FTY720) has a synergistic effect in the stimulation of an immune response as compared to administration of the VACVΔE5R virus or of the immune checkpoint blocking agent, anti-cancer drug, or fingolimod (FTY720) alone.

In one aspect, the present disclosure provides a nucleic acid encoding the engineered VACVΔE5R viruses of the present technology.

In one aspect, the present disclosure provides a kit comprising the engineered VACVΔE5R viruses of the present technology, and instructions for use.

In one aspect, the present disclosure provides a myxoma virus (MYXV) genetically engineered to comprise a mutant M31R gene (MYXVΔM31R). In some embodiments, the virus further comprises a heterologous nucleic acid molecule encoding one or more of OX40L, hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or a deletion of any one or more of myxoma orthologs of vaccinia virus thymidine kinase (TK), C7 (ΔC7L), E3L (ΔE3L), E3LΔ83N, B2R (ΔB2R), B19R (B18R; ΔWR200), IL18BP, K7R, C12L, B8R, B14R, N1L, C11R, K1L, M1L, N2L, or WR199. In some embodiments, the mutant M31R gene comprises replacement of at least a portion of the gene with one or more gene cassettes comprising the heterologous nucleic acid molecule. In some embodiments, the one or more gene cassettes comprises a heterologous nucleic acid molecule encoding OX40L (MYXVΔM31R-OX40L). In some embodiments, the one or more gene cassettes further comprises a heterologous nucleic acid molecule encoding human Fms-like tyrosine kinase 3 ligand (hFlt3L) (MYXVΔM31R-OX40L-hFlt3L). In some embodiments, the one or more gene cassettes comprises a heterologous nucleic acid molecule encoding human Fms-like tyrosine kinase 3 ligand (hFlt3L) (MYXVΔM31R-hFlt3L). In some embodiments, the heterologous nucleic acid is expressed from within a myxoma ortholog of a vaccinia viral gene selected from the group consisting of the thymidine kinase (TK) gene, the C7 gene, the C11 gene, the K3 gene, the F1 gene, the F2 gene, the F4 gene, the F6 gene, the F8 gene, the F9 gene, the F11 gene, the F14.5 gene, the J2 gene, the A46 gene, the E3L gene, the B18R gene (WR200), the E5R gene, the K7R gene, the C12L gene, the B8R gene, the B14R gene, the N1L gene, the K1L gene, the C16 gene, the M1L gene, the N2L gene, and the WR199 gene. In some embodiments, the virus further comprises a mutant myxoma ortholog of vaccinia virus thymidine kinase (TK) gene. In some embodiments, the mutant TK gene comprises replacement of at least a portion of the gene with one or more gene cassettes comprising a heterologous nucleic acid molecule. In some embodiments, the virus further comprises a mutant myxoma ortholog of vaccinia virus C7 gene. In some embodiments, the mutant C7 gene comprises an insertion of one or more gene cassettes comprising a heterologous nucleic acid molecule. In some embodiments, the mutant C7 gene comprises replacement of all or at least a portion of the gene with one or more gene cassettes comprising a heterologous nucleic acid molecule.

In one aspect, the present disclosure provides an immunogenic composition comprising the MYXVΔM31R virus. In some embodiments, the immunogenic composition further comprises a pharmaceutically acceptable carrier. In some embodiments, the immunogenic composition further comprises a pharmaceutically acceptable adjuvant.

In one aspect, the present disclosure provides a method for treating a solid tumor in a subject in need thereof, the method comprising delivering to a tumor a composition comprising an effective amount of the MYXVΔM31R virus or the immunogenic composition. In some embodiments, the treatment comprises one or more of the following: inducing an immune response in the subject against the tumor or enhancing or promoting an ongoing immune response against the tumor in the subject, reducing the size of the tumor, eradicating the tumor, inhibiting the growth of the tumor, inhibiting metastatic growth of the tumor, inducing apoptosis of tumor cells, or prolonging survival of the subject. In some embodiments, the composition is administered by intratumoral or intravenous injection or a simultaneous or sequential combination of intratumoral and intravenous injection. In some embodiments, the tumor is melanoma, colon, breast, bladder, or prostate carcinoma.

In some embodiments, the composition further comprises one or more agents selected from: one or more immune checkpoint blocking agents; one or more anti-cancer drugs; fingolimod (FTY720); and any combination thereof. In some embodiments, the method further comprises separately, sequentially, or simultaneously administering to the subject one or more agents selected from: one or more immune checkpoint blocking agents; one or more anti-cancer drugs, fingolimod (FTY720); and any combination thereof. In some embodiments, the one or more immune checkpoint blocking agents is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof; and/or the one or more anti-cancer drugs is selected from the group consisting of a Mek inhibitor (U0126, selumitinib (AZD6244), PD98059, trametinib, cobimetinib), an EGFR inhibitor (lapatinib (LPN), erlotinib (ERL)), a HER2 inhibitor (lapatinib (LPN), Trastuzumab), a Raf inhibitor (sorafenib (SFN)), a BRAF inhibitor (dabrafenib, vemurafenib), an anti-OX40 antibody, a GITR agonist antibody, an anti-CSFR antibody, a CSFR inhibitor, paclitaxel, TLR9 agonist CpG, and a VEGF inhibitor (Bevacizumab), and any combination thereof. In some embodiments, the one or more immune checkpoint blocking agents comprises anti-PD-L1 antibody. In some embodiments, the one or more immune checkpoint blocking agents comprises anti-PD-1 antibody. In some embodiments, the one or more immune checkpoint blocking agents comprises anti-CTLA-4 antibody. In some embodiments, the combination of the MYXVΔM31R virus with the immune checkpoint blocking agent, anti-cancer drug, and/or fingolimod (FTY720) has a synergistic effect in the treatment of the tumor as compared to administration of either the MYXVΔM31R virus or of the immune checkpoint blocking agent, anti-cancer drug, or fingolimod (FTY720) alone.

In one aspect, the present disclosure provides a method of stimulating an immune response comprising administering to a subject an effective amount of the virus or the immunogenic composition of. In some embodiments, the method further comprises separately, sequentially, or simultaneously administering to the subject one or more agents selected from: one or more immune checkpoint blocking agents; one or more anti-cancer drugs; fingolimod (FTY720); and any combination thereof. In some embodiments, the one or more immune checkpoint blocking agents is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof; and/or the one or more anti-cancer drugs is selected from the group consisting of a Mek inhibitor (U0126, selumitinib (AZD6244), PD98059, trametinib, cobimetinib), an EGFR inhibitor (lapatinib (LPN), erlotinib (ERL)), a HER2 inhibitor (lapatinib (LPN), Trastuzumab), a Raf inhibitor (sorafenib (SFN)), a BRAF inhibitor (dabrafenib, vemurafenib), an anti-OX40 antibody, a GITR agonist antibody, an anti-CSFR antibody, a CSFR inhibitor, paclitaxel, TLR9 agonist CpG, and a VEGF inhibitor (Bevacizumab), and any combination thereof. In some embodiments, the one or more immune checkpoint blocking agents comprises anti-PD-L1 antibody. In some embodiments, the one or more immune checkpoint blocking agents comprises anti-PD-1 antibody. In some embodiments, the one or more immune checkpoint blocking agents comprises anti-CTLA-4 antibody. In some embodiments, the combination of the MYXVΔM31R virus with the immune checkpoint blocking agent, anti-cancer drug, and/or fingolimod (FTY720) has a synergistic effect in the stimulation of an immune response as compared to administration of the MYXVΔM31R virus or of the immune checkpoint blocking agent, anti-cancer drug, or fingolimod (FTY720) alone.

In one aspect, the present disclosure provides a nucleic acid encoding the engineered MYXVΔM31R viruses of the present technology.

In one aspect, the present disclosure provides a kit comprising the engineered MYXVΔM31R viruses of the present technology, and instructions for use.

In one aspect, the present disclosure provides a recombinant modified vaccinia Ankara (MVA) virus comprising a mutant C7 gene and a heterologous nucleic acid molecule encoding OX40L (MVAΔC7L-OX40L). In some embodiments, the recombinant MVAΔC7L-OX40L virus further comprises a heterologous nucleic acid encoding human Fms-like tyrosine kinase 3 ligand (hFlt3L) (MVAΔC7L-hFlt3L-OX40L). In some embodiments, the recombinant MVAΔC7L-OX40L virus of the present technology further comprises a mutant thymidine kinase (TK) gene. In some embodiments, the recombinant MVAΔC7L-OX40L virus comprising the mutant TK gene comprises replacement of at least a portion of the gene with one or more gene cassettes comprising a heterologous nucleic acid molecule. In some embodiments, the one or more gene cassettes comprise the heterologous nucleic acid molecule encoding OX40L. In some embodiments, the mutant C7 gene comprises an insertion of one or more gene cassettes comprising a heterologous nucleic acid molecule. In some embodiments, the mutant C7 gene comprises replacement of all or at least a portion of the gene with one or more gene cassettes comprising a heterologous nucleic acid molecule. In some embodiments, the one or more gene cassettes comprise a heterologous nucleic acid molecule encoding hFlt3L. In some embodiments, the mutant C7 gene comprises replacement of at least a portion of the gene with one or more gene cassettes comprising the heterologous nucleic acid molecule encoding hFlt3L, and wherein the virus further comprises a mutant TK gene comprising replacement of at least a portion of the TK gene with one or more gene cassettes comprising the heterologous nucleic acid molecule encoding OX40L (MVAΔC7L-hFlt3L-TK(−)-OX40L). In some embodiments, the OX40L is expressed from within a MVA viral gene. In some embodiments, the OX40L is expressed from within a viral gene selected from the group consisting of the thymidine kinase (TK) gene, the C7 gene, the C11 gene, the K3 gene, the F1 gene, the F2 gene, the F4 gene, the F6 gene, the F8 gene, the F9 gene, the F11 gene, the F14.5 gene, the J2 gene, the A46 gene, the E3L gene, the B18R gene (WR200), the E5R gene, the K7R gene, the C12L gene, the B8R gene, the B14R gene, the N1L gene, the K1L gene, the C16 gene, the M1L gene, the N2L gene, and the WR199 gene. In some embodiments, the OX40L is expressed from within the TK gene. In some embodiments, the OX40L is expressed from within the TK gene and the hFlt3L is expressed from within the C7 gene. In some embodiments, the virus further comprises a heterologous nucleic acid molecule encoding one or more of hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or a deletion of any one or more of E3L (ΔE3L), E3LΔ83N, B2R (ΔB2R), B19R (B18R; ΔWR200), IL18BP, E5R, K7R, C12L, B8R, B14R, N1L, C11R, K1L, M1L, N2L, or WR199. In some embodiments, the recombinant MVA virus exhibits one or more of the following characteristics: induction of increased levels of effector T-cells in tumor cells as compared to tumor cells infected with the corresponding MVAΔC7L virus; induction of increased splenic production of effector T-cells as compared to the corresponding MVAΔC7L virus; and reduction of tumor volume in tumor cells contacted with the recombinant MVAΔC7L-OX40L virus as compared to tumor cells contacted with the corresponding MVAΔC7L virus. In some embodiments, the tumor cells comprise melanoma cells.

In another aspect, the present disclosure provides, an immunogenic composition comprising the recombinant MVAΔC7L-OX40L virus of the present technology. In some embodiments, the immunogenic composition further comprises a pharmaceutically acceptable carrier. In some embodiments, the immunogenic composition of the present technology comprises a pharmaceutically acceptable adjuvant.

In another aspect, the present disclosure provides a method for treating a solid tumor in a subject in need thereof, the method comprising delivering to a tumor a composition comprising an effective amount of the recombinant MVAΔC7L-the present technology. In some embodiments, the treatment comprises one or more of the following: inducing an immune response in the subject against the tumor or enhancing or promoting an ongoing immune response against the tumor in the subject, reducing the size of the tumor, eradicating the tumor, inhibiting the growth of the tumor, inhibiting metastatic growth of the tumor, inducing apoptosis of tumor cells, or prolonging survival of the subject. In some embodiments, the method for treating a solid tumor in a subject in need thereof comprises the induction, enhancement, or promotion of the immune response comprises one or more of the following: increased levels of effector T-cells in tumor cells as compared to tumor cells infected with the corresponding MVAΔC7L virus; and increased splenic production of effector T-cells as compared to the corresponding MVAΔC7L virus. In some embodiments, the method of treating a solid tumor in a subject in need thereof the composition is administered by intratumoral or intravenous injection or a simultaneous or sequential combination of intratumoral and intravenous injection. In some embodiments of the method of treating a solid tumor in a subject in need thereof, the tumor is melanoma, colon, breast, bladder, or prostate carcinoma. In some embodiments of the method of treating a solid tumor in a subject in need thereof, the composition comprises one or more immune checkpoint blocking agents. In some embodiments of the method of treating a solid tumor in a subject in need thereof, the method further comprises administering to the subject one or more immune checkpoint blocking agents. In some embodiments of the method of treating a solid tumor in a subject in need thereof, the one or more immune checkpoint blocking agent is selected from the group consisting of anti-PD-1 antibody, anti-PD-L1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof. In some embodiments of the method of treating a solid tumor in a subject in need thereof, the one or more immune checkpoint blocking agents comprises anti-PD-1 antibody. In some embodiments of the method of treating a solid tumor in a subject in need thereof, the one or more immune checkpoint blocking agents comprises anti-PD-L1 antibody. In some embodiments of the method of treating a solid tumor in a subject in need thereof, the one or more immune checkpoint blocking agents comprises anti-CTLA-4 antibody. In some embodiments of the method of treating a solid tumor in a subject in need thereof, the combination of the MVAΔC7L-OX40L or MVAΔC7L-hFlt3L-OX40L and the immune checkpoint blocking agent has a synergistic effect in the treatment of the tumor as compared to administration of either the MVAΔC7L-OX40L or MVAΔC7L-hFlt3L-OX40L or of the immune checkpoint blocking agent alone.

In another aspect, the present disclosure provides a method of stimulating an immune response comprising administering to a subject an effective amount of the virus of the present technology (e.g., MVAΔC7L-OX40L, MVAΔC7L-hFlt3L-OX40L) or an immunogenic composition of the present technology. In some embodiments, the method further comprises administering to the subject one or more immune checkpoint blocking agents. In some embodiments, the one or more immune checkpoint blocking agents is selected from the group consisting of anti-PD-1 antibody, anti-PD-L1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof. In some embodiments, the one or more immune checkpoint blocking agents comprises anti-PD-1 antibody. In some embodiments, the one or more immune checkpoint blocking agents comprises anti-PD-L1 antibody. In some embodiments, the one or more immune checkpoint blocking agents comprises anti-CTLA-4 antibody.

In another aspect, the present disclosure provides, a recombinant modified vaccinia Ankara (MVA) virus comprising a mutant E3 gene and a heterologous nucleic acid molecule encoding OX40L (MVAΔE3L-OX40L). In some embodiments, the recombinant MVAΔE3L-OX40L virus comprises a mutant thymidine kinase (TK) gene. In some embodiments, the mutant TK gene comprises replacement of at least a portion of the gene with one or more gene cassettes comprising a heterologous nucleic acid molecule. In some embodiments, the one or more gene cassettes comprise the heterologous nucleic acid molecule encoding OX40L. In some embodiments of the virus of the present technology, the OX40L is expressed from within a MVA viral gene. In some embodiments of the virus of the present technology, the OX40L is expressed from within a viral gene selected from the group consisting of the thymidine kinase (TK) gene, the C7 gene, the C11 gene, the K3 gene, the F1 gene, the F2 gene, the F4 gene, the F6 gene, the F8 gene, the F9 gene, the F11 gene, the F14.5 gene, the J2 gene, the A46 gene, the E3L gene, the B18R gene (WR200), the E5R gene, the K7R gene, the C12L gene, the B8R gene, the B14R gene, the N1L gene, the K1L gene, the C16 gene, the M1L gene, the N2L gene, and the WR199 gene. In some embodiments, of the virus of the present technology, the OX40L is expressed from within the TK gene. In some embodiments, of the virus of the present technology, the virus comprises a heterologous nucleic acid molecule encoding one or more of hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or a deletion of any one or more of E3LΔ83N, B2R (ΔB2R), B19R (B18R; ΔWR200), E5R, K7R, C12L (IL18BP), B8R, B14R, N1L, C11R, K1L, M1L, N2L, or WR199. In some embodiments, of the virus of the present technology, the recombinant MVA virus exhibits one or more of the following characteristics: induction of increased levels of effector T-cells in tumor cells as compared to tumor cells infected with the corresponding MVAΔE3L virus; induction of increased splenic production of effector T-cells as compared to the corresponding MVAΔE3L virus; and reduction of tumor volume in tumor cells contacted with the recombinant MVAΔE3L-OX40L virus as compared to tumor cells contacted with the corresponding MVAΔE3L virus. In some embodiments, of the virus of the present technology, the tumor cells comprise melanoma cells. In some embodiments, the mutant E3 gene is at least partially deleted, is not expressed, is expressed at levels so low as to have no effect, or expressed as a non-functional protein. In some embodiments, the mutant TK gene is at least partially deleted, is not expressed, is expressed at levels so low as to have no effect, or expressed as a non-functional protein.

In another aspect, the present disclosure provides an immunogenic composition comprising the recombinant MVAΔE3L-OX40L virus of the present technology. In some embodiments, the immunogenic composition of the present technology comprises a pharmaceutically acceptable carrier. In some embodiments, the immunogenic composition of the present technology comprises a pharmaceutically acceptable adjuvant.

In another aspect, the present disclosure provided a method for treating a solid tumor in a subject in need thereof, the method comprising delivering to a tumor a composition comprising an effective amount of the recombinant MVAΔE3L-OX40L virus of the present technology or an immunogenic composition of the present technology. In some embodiments of the method for treating a solid tumor in a subject in need thereof, the treatment comprises one or more of the following: inducing an immune response in the subject against the tumor or enhancing or promoting an ongoing immune response against the tumor in the subject, reducing the size of the tumor, eradicating the tumor, inhibiting the growth of the tumor, inhibiting metastatic growth of the tumor, inducing apoptosis of tumor cells, or prolonging survival of the subject. In some embodiments of the method for treating a solid tumor in a subject in need thereof, the induction, enhancement, or promotion of the immune response comprises one or more of the following: increased levels of effector T-cells in tumor cells as compared to tumor cells infected with the corresponding MVAΔE3L virus; and increased splenic production of effector T-cells as compared to the corresponding MVAΔE3L virus. In some embodiments of the method for treating a solid tumor in a subject in need thereof, the composition is administered by intratumoral or intravenous injection or a simultaneous or sequential combination of intratumoral and intravenous injection. In some embodiments of the method for treating a solid tumor in a subject in need thereof, the tumor is melanoma, colon, breast, bladder, or prostate carcinoma. In some embodiments of the method for treating a solid tumor in a subject in need thereof, the composition further comprises one or more immune checkpoint blocking agents. In some embodiments of the method for treating a solid tumor in a subject in need thereof, the one or more immune checkpoint blocking agent is selected from the group consisting of anti-PD-1 antibody, anti-PD-L1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof. In some embodiments of the method for treating a solid tumor in a subject in need thereof, the one or more immune checkpoint blocking agents comprises anti-PD-1 antibody. In some embodiments of the method for treating a solid tumor in a subject in need thereof, the one or more immune checkpoint blocking agents comprises anti-PD-L1 antibody. In some embodiments of the method for treating a solid tumor in a subject in need thereof, the one or more immune checkpoint blocking agents comprises anti-CTLA-4 antibody. In some embodiments of the method for treating a solid tumor in a subject in need thereof, the combination of the MVAΔE3L-OX40L and the immune checkpoint blocking agent has a synergistic effect in the treatment of the tumor as compared to administration of either MVAΔE3L-OX40L or the immune checkpoint blocking agent alone.

In another aspect, the present disclosure provides a method of stimulating an immune response comprising administering to a subject an effective amount of a virus of the present technology (e.g., MVAΔE3L-OX40L) or an immunogenic composition of the present technology. In some embodiments, the method further comprises administering one or more immune checkpoint blocking agents to the subject. In some embodiments, the one or more immune checkpoint blocking agents is selected from the group consisting of anti-PD-1 antibody, anti-PD-L1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof. In some embodiments, the one or more immune checkpoint blocking agents comprises anti-PD-1 antibody. In some embodiments, the one or more immune checkpoint blocking agents comprises anti-PD-L1 antibody. In some embodiments, the one or more immune checkpoint blocking agents comprises anti-CTLA-4 antibody. In some embodiments, the combination of MVAΔE3L-OX40L and the immune checkpoint blocking agent has a synergistic effect in the treatment of the tumor as compared to administration of either MVAΔE3L-OX40L or the immune checkpoint blocking agent alone.

In another aspect, the present disclosure provides a recombinant vaccinia virus (VACV) comprising a mutant C7 gene and a heterologous nucleic acid molecule encoding OX40L (VACVΔC7L-OX40L). In some embodiments, the virus comprises a heterologous nucleic acid encoding human Fms-like tyrosine kinase 3 ligand (hFlt3L) (VACVΔC7L-hFlt3L-OX40L). In some embodiments, the virus comprises a mutant thymidine kinase (TK) gene. In some embodiments, the mutant TK gene comprises replacement of at least a portion of the gene with one or more gene cassettes comprising a heterologous nucleic acid molecule.

In some embodiments, the one or more gene cassettes comprise the heterologous nucleic acid molecule encoding OX40L. In some embodiments, the mutant C7 gene comprises an insertion of one or more gene cassettes comprising a heterologous nucleic acid molecule. In some embodiments, the mutant C7 gene comprises replacement of all or at least a portion of the gene with one or more gene cassettes comprising a heterologous nucleic acid molecule.

In some embodiments, the one or more gene cassettes comprise a heterologous nucleic acid molecule encoding hFlt3L. In some embodiments, the mutant C7 gene comprises replacement of at least a portion of the gene with one or more gene cassettes comprising the heterologous nucleic acid molecule encoding hFlt3L, and wherein the virus further comprises a mutant TK gene comprising replacement of at least a portion of the TK gene with one or more gene cassettes comprising the heterologous nucleic acid molecule encoding OX40L (VACVΔC7L-hFlt3L-TK(−)-OX40L). In some embodiments, the OX40L is expressed from within a vaccinia viral gene. In some embodiments, the OX40L is expressed from within a viral gene selected from the group consisting of the thymidine kinase (TK) gene, the C7 gene, the C11 gene, the K3 gene, the F1 gene, the F2 gene, the F4 gene, the F6 gene, the F8 gene, the F9 gene, the F11 gene, the F14.5 gene, the J2 gene, the A46 gene, the E3L gene, the B18R gene (WR200), the E5R gene, the K7R gene, the C12L gene, the B8R gene, the B14R gene, the N1L gene, the K1L gene, the C16 gene, the M1L gene, the N2L gene, and the WR199 gene. In some embodiments, the OX40L is expressed from within the TK gene. In some embodiments, the OX40L is expressed from within the TK gene and the hFlt3L is expressed from within the C7 gene. In some embodiments, the virus further comprises a heterologous nucleic acid molecule encoding one or more of hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or a deletion of any one or more of E3L (ΔE3L), E3LΔ83N, B2R (ΔB2R), B19R (B18R; ΔWR200), E5R, K7R, C12L (IL18BP), B8R, B14R, N1L, C11R, K1L, M1L, N2L, or WR199. In some embodiments, the virus further comprises a heterologous nucleic acid encoding hIL-12 and a heterologous nucleic acid encoding anti-huCTLA-4 (VACVΔC7L-anti-huCTLA-4-hFlt3L-OX40L-hIL-12). In some embodiments, the recombinant VACVΔC7L-OX40L virus exhibits one or more of the following characteristics: induction of increased levels of effector T-cells in tumor cells as compared to tumor cells infected with the corresponding VACVΔC7L virus; induction of increased splenic production of effector T-cells as compared to the corresponding VACVΔC7L virus; and reduction of tumor volume in tumor cells contacted with the recombinant VACVΔC7L-OX40L virus as compared to tumor cells contacted with the corresponding VACVΔC7L virus. In some embodiments, the tumor cells comprise melanoma cells.

In another aspect, the present disclosure provides, an immunogenic composition comprising the recombinant VACVΔC7L-OX40L virus of the present technology. In some embodiments, the immunogenic composition comprises a pharmaceutically acceptable carrier. In some embodiments, the immunogenic composition comprises a pharmaceutically acceptable adjuvant.

In another aspect, the present disclosure provides a method for treating a solid tumor in a subject in need thereof, the method comprising delivering to a tumor a composition comprising an effective amount of the recombinant VACVΔC7L-OX40L virus of the present technology or the immunogenic composition of the present technology. In some embodiments of the method for treating a solid tumor in a subject in need thereof, the treatment comprises one or more of the following: inducing an immune response in the subject against the tumor or enhancing or promoting an ongoing immune response against the tumor in the subject, reducing the size of the tumor, eradicating the tumor, inhibiting the growth of the tumor, inhibiting metastatic growth of the tumor, inducing apoptosis of tumor cells, or prolonging survival of the subject. In some embodiments of the method for treating a solid tumor in a subject in need thereof, the treatment comprises the induction, enhancement, or promotion of the immune response comprises one or more of the following: increased levels of effector T-cells in tumor cells as compared to tumor cells infected with the corresponding VACVΔC7L virus; and increased splenic production of effector T-cells as compared to the corresponding VACVΔC7L virus. In some embodiments of the method for treating a solid tumor in a subject in need thereof, the composition is administered by intratumoral or intravenous injection or a simultaneous or sequential combination of intratumoral and intravenous injection. In some embodiments of the method for treating a solid tumor in a subject in need thereof, the tumor is melanoma, colon, breast, bladder, or prostate carcinoma. In some embodiments of the method for treating a solid tumor in a subject in need thereof, the composition further comprises one or more immune checkpoint blocking agents. In some embodiments, the method further comprises administering to the subject one or more immune checkpoint blocking agents. In some embodiments the one or more immune checkpoint blocking agents is selected from the group consisting of anti-PD-1 antibody, anti-PD-L1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof. In some embodiments of the method for treating a solid tumor in a subject in need thereof, the one or more immune checkpoint blocking agents comprises the one or more immune checkpoint blocking agent comprises anti-PD-1 antibody. In some embodiments of the method for treating a solid tumor in a subject in need thereof, the one or more immune checkpoint blocking agents comprises the one or more immune checkpoint blocking agent comprises anti-PD-L1 antibody. In some embodiments of the method for treating a solid tumor in a subject in need thereof, comprises the one or more immune checkpoint blocking agent comprises anti-CTLA-4 antibody. In some embodiments of the method for treating a solid tumor in a subject in need thereof, the combination of the VACVΔC7L-OX40L or VACVΔC7L-hFlt3L-OX40L and the immune checkpoint blocking agent has a synergistic effect in the treatment of the tumor as compared to administration of VACVΔC7L-OX40L or VACVΔC7L-hFlt3L-OX40L or of the immune checkpoint blocking agent alone.

In another aspect, the present disclosure provides, a method for stimulating an immune response comprising administering to a subject an effective amount of the virus of the present technology (e.g., VACVΔC7L-OX40L or VACVΔC7L-hFlt3L-OX40L) or an immunogenic composition of the present technology. In some embodiments, the method further comprises administering one or more immune checkpoint blocking agents. In some embodiments, the one or more immune checkpoint blocking agents is selected from the group consisting of anti-PD-1 antibody, anti-PD-L1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof. In some embodiments, the immune checkpoint blocking agent comprises anti-PD-1 antibody. In some embodiments, the immune checkpoint blocking agent comprises anti-PD-L1 antibody. In some embodiments, the immune checkpoint blocking agent comprises anti-CTLA-4 antibody.

In another aspect, the present disclosure provides, a recombinant modified vaccinia Ankara (MVA) virus nucleic acid sequence, wherein the nucleic acid sequence between position 75,560 and 76,093 of SEQ ID NO: 1 is replaced with a heterologous nucleic acid sequence comprising an open reading frame that encodes OX40L, and wherein the MVA further comprises a C7 mutant. In some embodiments of the MVA virus of the present technology, the nucleic acid sequence between position 18,407 and 18,859 of SEQ ID NO: 1 is replaced with a heterologous nucleic acid sequence comprising an open reading frame that encodes human Fms-like tyrosine kinase 3 ligand (hFlt3L).

In another aspect, the present disclosure provides a recombinant modified vaccinia Ankara (MVA) virus nucleic acid sequence, wherein the nucleic acid sequence between position 75,798 to 75,868 of SEQ ID NO: 1 is replaced with a heterologous nucleic acid sequence comprising an open reading frame that encodes OX40L or encodes human Fms-like tyrosine kinase 3 ligand (hFlt3L), and wherein the MVA further comprises an E3 mutant.

In another aspect, the present disclosure provides a recombinant vaccinia virus (VACV) nucleic acid sequence, wherein the nucleic acid sequence between position 80,962 and 81,032 of SEQ ID NO: 2 is replaced with a heterologous nucleic acid sequence comprising an open reading frame that encodes OX40L or, and wherein the VACV further comprises a C7 mutant. In some embodiments the recombinant VACV of the present technology the nucleic acid sequence between position 15,716 and 16,168 of SEQ ID NO: 2 is replaced with a heterologous nucleic acid sequence comprising an open reading frame that encodes human Fms-like tyrosine kinase 3 ligand (hFlt3L).

In another aspect, the present disclosure provides a nucleic acid sequence encoding the recombinant MVAΔC7L-OX40L virus of the present technology.

In another aspect, the present disclosure provides a nucleic acid sequence encoding the recombinant MVAΔE3L-OX40L virus of the present technology.

In another aspect, the present disclosure provides a nucleic acid sequence encoding the recombinant VACVΔC7L-OX40L virus of any one of the present technology.

In another aspect, the present disclosure provides a kit comprising the recombinant MVAΔC7L-OX40L virus of the present technology or the immunogenic composition of the present technology, and instructions for use.

In another aspect, the present disclosure provides a kit comprising the recombinant MVAΔE3L-OX40L virus of any one of the present technology or the immunogenic composition of the present technology, and instructions for use.

In another aspect, the present disclosure provides a kit comprising the recombinant VACVΔC7L-OX40L virus of the present technology or the immunogenic composition of the present technology, and instructions for use.

In some embodiments, the present disclosure provides a recombinant MVAΔC7L-OX40L virus wherein the mutant C7 gene is at least partially deleted, is not expressed, is expressed at levels so low as to have no effect, or expressed as a non-functional protein. In some embodiments, the present disclosure provides a recombinant MVAΔC7L-OX40L virus, wherein the mutant TK gene is at least partially deleted, is not expressed, is expressed at levels so low as to have no effect, or expressed as a non-functional protein. In some embodiments, the present disclosure provides a recombinant MVAΔE3L-OX40L virus wherein the mutant E3 gene is at least partially deleted, is not expressed, is expressed at levels so low as to have no effect, or expressed as a non-functional protein. In some embodiments, the present disclosure provides a recombinant MVAΔE3L-OX40L virus wherein the mutant TK gene is at least partially deleted, is not expressed, is expressed at levels so low as to have no effect, or expressed as a non-functional protein. In some embodiments, the present technology provides a recombinant VACVΔC7L-OX40L virus wherein the mutant C7 gene is at least partially deleted, is not expressed, is expressed at levels so low as to have no effect, or expressed as a non-functional protein. In some embodiments, the present disclosure provides a recombinant VACVΔC7L-OX40L virus wherein the mutant TK gene is at least partially deleted, is not expressed, is expressed at levels so low as to have no effect, or expressed as a non-functional protein.

In another aspect, the present disclosure provides a method for treating a solid tumor in a subject in need thereof, the method comprising administering to the subject an antigen and a therapeutically effective amount of an adjuvant comprising a recombinant modified vaccinia Ankara (MVA) virus comprising a mutant C7 gene and a heterologous nucleic acid encoding OX40L (MVAΔC7L-OX40L). In some embodiments, the MVAΔC7L-OX40L virus further comprises a heterologous nucleic acid encoding human Fms-like tyrosine kinase 3 ligand (hFlt3L) (MVAΔC7L-hFlt3L-OX40L). In some embodiments, the virus further comprises a mutant thymidine kinase (TK) gene. In some embodiments, the mutant TK gene comprises replacement of at least a portion of the gene with one or more gene cassettes comprising a heterologous nucleic acid molecule. In some embodiments, the one or more gene cassettes comprise the heterologous nucleic acid molecule encoding OX40L. In some embodiments, the mutant C7 gene comprises an insertion of one or more gene cassettes comprising a heterologous nucleic acid molecule. In some embodiments, the mutant C7 gene comprises replacement of all or at least a portion of the gene with one or more gene cassettes comprising a heterologous nucleic acid. In some embodiments, the one or more gene cassettes comprise a heterologous nucleic acid molecule encoding hFlt3L. In some embodiments, the mutant C7 gene comprises replacement of at least a portion of the gene with one or more gene cassettes comprising the heterologous nucleic acid molecule encoding hFlt3L, and wherein the virus further comprises a mutant TK gene comprising replacement of at least a portion of the TK gene with one or more gene cassettes comprising the heterologous nucleic acid molecule encoding OX40L (MVAΔC7L-hFlt3L-TK(−)-OX40L). In some embodiments, the OX40L is expressed from within a MVA viral gene. In some embodiments, the OX40L is expressed from within a viral gene selected from the group consisting of the thymidine kinase (TK) gene, the C7 gene, the C11 gene, the K3 gene, the F1 gene, the F2 gene, the F4 gene, the F6 gene, the F8 gene, the F9 gene, the F11 gene, the F14.5 gene, the J2 gene, the A46 gene, the E3L gene, the B18R gene (WR200), the E5R gene, the K7R gene, the C12L gene, the B8R gene, the B14R gene, the N1L gene, the K1L gene, the C16 gene, the M1L gene, the N2L gene, and the WR199 gene. In some embodiments, the OX40L is expressed from within the TK gene. In some embodiments, the OX40L is expressed from within the TK gene and the hFlt3L is expressed from within the C7 gene. In some embodiments, the virus further comprises a heterologous nucleic acid molecule encoding one or more of hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or a deletion of any one or more of E3L (ΔE3L), E3LΔ83N, B2R (ΔB2R), B19R (B18R; ΔWR200), IL18BP, E5R, K7R, C12L, B8R, B14R, N1L, C11R, K1L, M1L, N2L, or WR199.

In some embodiments, the antigen is selected from the group consisting of tumor differentiation antigens, cancer testis antigens, neoantigens, viral antigens in the case of tumors associated with oncogenic virus infection, GPA33, HER2/neu, GD2, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, tyrosinase-related proteins 1 and 2, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, p53, kras, lung resistance protein (LRP) Bcl-2, prostate specific antigen (PSA), Ki-67, CEACAM6, colon-specific antigen-p (CSAp), NY-ESO-1, human papilloma virus E6 and E7, and any combination thereof.

In some embodiments, the administration step comprises administering the antigen and adjuvant in one or more doses and/or wherein the antigen and adjuvant are administered separately, sequentially, or simultaneously.

In some embodiments, the method further comprises administering to the subject an immune checkpoint blockade agent selected from the group consisting of anti-PD-1 antibody, anti-PD-L1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof.

In some embodiments, the antigen and adjuvant are delivered to the subject separately, sequentially, or simultaneously with the administration of the immune checkpoint blockade agent.

In some embodiments, the treatment comprises one or more of the following: inducing an immune response in the subject against the tumor or enhancing or promoting an ongoing immune response against the tumor in the subject, reducing the size of the tumor, eradicating the tumor, inhibiting growth of the tumor, inhibiting metastatic growth of the tumor, inducing apoptosis of the tumor cells, or prolonging survival of the subject. In some embodiments, the induction, enhancement, or promotion of the immune response comprises one or more of the following: (i) increased levels of interferon gamma (IFN-γ) expression in T-cells in the spleen, draining lymph nodes, and/or serum as compared to an untreated control sample; (ii) increased levels of antigen-specific T-cells in the spleen, draining lymph nodes, and/or serum as compared to an untreated control sample; and (iii) increased levels of antigen-specific immunoglobulin in serum as compared to an untreated control sample. In some embodiments, the antigen-specific immunoglobulin is IgG1 or IgG2.

In some embodiments, the antigen and adjuvant are formulated to be administered intratumorally, intramuscularly, intradermally, or subcutaneously.

In some embodiments, the tumor is selected from the group consisting of melanoma, colorectal cancer, breast cancer, bladder cancer, prostate cancer, lung cancer, pancreatic cancer, ovarian cancer, squamous cell carcinoma of the skin, Merkel cell carcinoma, gastric cancer, liver cancer, and sarcoma.

In some embodiments, the MVAΔC7L-OX40L virus is administered at a dosage per administration of about 10⁵ to about 10¹⁰ plaque-forming units (pfu).

In some embodiments of the method, the subject is human.

In one aspect, the present disclosure provides an immunogenic composition comprising the antigen and the adjuvant of the present technology. In some embodiments, the immunogenic composition further comprises a pharmaceutically acceptable carrier. In some embodiments, the antigen is selected from the group consisting of tumor differentiation antigens, cancer testis antigens, neoantigens, viral antigens in the case of tumors associated with oncogenic virus infection, GPA33, HER2/neu, GD2, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, tyrosinase-related proteins 1 and 2, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, p53, kras, lung resistance protein (LRP) Bcl-2, prostate specific antigen (PSA), Ki-67, CEACAM6, colon-specific antigen-p (CSAp), NY-ESO-1, human papilloma virus E6 and E7, and any combination thereof. In some embodiments, the immunogenic composition further comprises an immune checkpoint blockade agent selected from the group consisting of anti-PD-1 antibody, anti-PD-L1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof.

In one aspect, the present disclosure provides a kit comprising instructions for use, a container means, and a separate portion of each of: (a) an antigen; and (b) an adjuvant of the present technology. In some embodiments of the kit, the antigen is selected from the group consisting of tumor differentiation antigens, cancer testis antigens, neoantigens, viral antigens in the case of tumors associated with oncogenic virus infection, GPA33, HER2/neu, GD2, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, tyrosinase-related proteins 1 and 2, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, p53, kras, lung resistance protein (LRP) Bcl-2, prostate specific antigen (PSA), Ki-67, CEACAM6, colon-specific antigen-p (CSAp), NY-ESO-1, human papilloma virus E6 and E7, and any combination thereof.

In some embodiments, the kit further comprises (c) an immune checkpoint blockade agent selected from the group consisting of anti-PD-1 antibody, anti-PD-L1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof.

In some embodiments of the methods of the present technology, the antigen is a neoantigen selected from the group consisting of M27 (REGVELCPGNKYEMRRHGTTHSLVIHD) (SEQ ID NO: 17), M30 (PSKPSFQEFVDWENVSPELNSTDQPFL) (SEQ ID NO: 18), M48 (SHCHWNDLAVIPAGVVHNWDFEPRKVS) (SEQ ID NO: 19), and combinations thereof.

In some embodiments of the immunogenic compositions of the present technology, the antigen is a neoantigen selected from the group consisting of M27 (REGVELCPGNKYEMRRHGTTHSLVIHD) (SEQ ID NO: 17), M30 (PSKPSFQEFVDWENVSPELNSTDQPFL) (SEQ ID NO: 18), M48 (SHCHWNDLAVIPAGVVHNWDFEPRKVS) (SEQ ID NO: 19), and combinations thereof.

In some embodiments of the kit of the present technology, the antigen is a neoantigen selected from the group consisting of M27 (REGVELCPGNKYEMRRHGTTHSLVIHD) (SEQ ID NO: 17), M30 (PSKPSFQEFVDWENVSPELNSTDQPFL) (SEQ ID NO: 18), M48 (SHCHWNDLAVIPAGVVHNWDFEPRKVS) (SEQ ID NO: 19), and combinations thereof.

In one aspect, the present disclosure provides a modified vaccinia Ankara (MVA) virus genetically engineered to comprise a mutant E5R gene (MVAΔE5R). In some embodiments, the virus further comprises a heterologous nucleic acid molecule encoding one or more of OX40L, hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or a deletion of any one or more of thymidine kinase (TK), C7 (ΔC7L), E3L (ΔE3L), E3LΔ83N, B2R (ΔB2R), B19R (B18R; ΔWR200), IL18BP, K7R, C12L, B8R, B14R, N1L, C11R, K1L, M1L, N2L, or WR199. In some embodiments, the mutant E5R gene comprises replacement of at least a portion of the gene with one or more gene cassettes comprising the heterologous nucleic acid molecule. In some embodiments, the one or more gene cassettes comprises a heterologous nucleic acid molecule encoding OX40L (MVAΔE5R-OX40L). In some embodiments, the one or more gene cassettes further comprises a heterologous nucleic acid molecule encoding human Fms-like tyrosine kinase 3 ligand (hFlt3L) (MVAΔE5R-OX40L-hFlt3L). In some embodiments, the one or more gene cassettes comprises a heterologous nucleic acid molecule encoding human Fms-like tyrosine kinase 3 ligand (hFlt3L) (MVAΔE5R-hFlt3L). In some embodiments, the heterologous nucleic acid is expressed from within a viral gene selected from the group consisting of the thymidine kinase (TK) gene, the C7 gene, the C11 gene, the K3 gene, the F1 gene, the F2 gene, the F4 gene, the F6 gene, the F8 gene, the F9 gene, the F11 gene, the F14.5 gene, the J2 gene, the A46 gene, the E3L gene, the B18R gene (WR200), the E5R gene, the K7R gene, the C12L gene, the B8R gene, the B14R gene, the N1L gene, the K1L gene, the C16 gene, the M1L gene, the N2L gene, and the WR199 gene. In some embodiments, the virus further comprises a mutant thymidine kinase (TK) gene. In some embodiments, the mutant TK gene comprises replacement of at least a portion of the gene with one or more gene cassettes comprising a heterologous nucleic acid molecule. In some embodiments, the virus further comprises a mutant C7 gene. In some embodiments, the mutant C7 gene comprises an insertion of one or more gene cassettes comprising a heterologous nucleic acid molecule. In some embodiments, the mutant C7 gene comprises replacement of all or at least a portion of the gene with one or more gene cassettes comprising a heterologous nucleic acid molecule. In some embodiments, the MVAΔE5R-OX40L-hFlt3L virus further comprises a mutant C11R gene (MVAΔE5R-OX40L-hFlt3L-ΔC11R). In some embodiments, the mutant C11R gene comprises an insertion of one or more gene cassettes comprising a heterologous nucleic acid molecule. In some embodiments, the mutant C11R gene comprises replacement of all or at least a portion of the gene with one or more gene cassettes comprising a heterologous nucleic acid molecule. In some embodiments, the MVAΔE5R-OX40L-hFlt3L virus further comprises a mutant WR199 gene (MVAΔE5R-OX40L-hFlt3L-ΔWR199). In some embodiments, the mutant WR199 gene comprises an insertion or one or more gene cassettes comprising a heterologous nucleic acid molecule. In some embodiments, the mutant WR199 gene comprises replacement of all or at least a portion of the gene with one or more gene cassettes comprising a heterologous nucleic acid molecule. In some embodiments, the MVAΔE5R virus further comprises a mutant E3L gene (ΔE3L). In some embodiments, the mutant E3L gene comprises an insertion of one or more gene cassettes comprising a heterologous nucleic acid molecule. In some embodiments, the mutant E3L gene comprises replacement of all or at least a portion of the gene with one or more gene cassettes comprising a heterologous nucleic acid molecule. In some embodiments, the one or more gene cassettes comprises a heterologous nucleic acid molecule encoding OX40L. In some embodiments, the one or more gene cassettes further comprises a heterologous nucleic acid molecule encoding human Fms-like tyrosine kinase 3 ligand (hFlt3L). In some embodiments, the one or more gene cassettes comprises a heterologous nucleic acid encoding human Fms-like typrsine kinase 3 ligand (hFlt3L).

In one aspect, the present disclosure provides an immunogenic composition comprising the MVAΔE5R virus. In some embodiments, the immunogenic composition further comprises a pharmaceutically acceptable carrier. In some embodiments, the immunogenic composition further comprises a pharmaceutically acceptable adjuvant.

In one aspect, the present disclosure provides a method for treating a solid tumor in a subject in need thereof, the method comprising delivering to a tumor a composition comprising an effective amount of the MVAΔE5R virus or the immunogenic composition. In some embodiments, the treatment comprises one or more of the following: inducing an immune response in the subject against the tumor or enhancing or promoting an ongoing immune response against the tumor in the subject, reducing the size of the tumor, eradicating the tumor, inhibiting the growth of the tumor, inhibiting metastatic growth of the tumor, inducing apoptosis of tumor cells, or prolonging survival of the subject. In some embodiments, the composition is administered by intratumoral or intravenous injection or a simultaneous or sequential combination of intratumoral and intravenous injection. In some embodiments, the tumor is melanoma, colon, breast, bladder, or prostate carcinoma. In some embodiments, the composition further comprises one or more agents selected from: one or more immune checkpoint blocking agents; one or more anti-cancer drugs; fingolimod (FTY720); and any combination thereof.

In some embodiments, the method further comprises separately, sequentially, or simultaneously administering to the subject one or more agents selected from: one or more immune checkpoint blocking agents; one or more anti-cancer drugs, fingolimod (FTY720); and any combination thereof. In some embodiments, the one or more immune checkpoint blocking agents is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof; and/or the one or more anti-cancer drugs is selected from the group consisting of a Mek inhibitor (U0126, selumitinib (AZD6244), PD98059, trametinib, cobimetinib), an EGFR inhibitor (lapatinib (LPN), erlotinib (ERL)), a HER2 inhibitor (lapatinib (LPN), Trastuzumab), a Raf inhibitor (sorafenib (SFN)), a BRAF inhibitor (dabrafenib, vemurafenib), an anti-OX40 antibody, a GITR agonist antibody, an anti-CSFR antibody, a CSFR inhibitor, paclitaxel, TLR9 agonist CpG, and a VEGF inhibitor (Bevacizumab), and any combination thereof. In some embodiments, the one or more immune checkpoint blocking agents comprises anti-PD-L1 antibody. In some embodiments, the one or more immune checkpoint blocking agents comprises anti-PD-1 antibody. In some embodiments, the one or more immune checkpoint blocking agents comprises anti-CTLA-4 antibody. In some embodiments, the combination of the MVAΔE5R virus with the immune checkpoint blocking agent, anti-cancer drug, and/or fingolimod (FTY720) has a synergistic effect in the treatment of the tumor as compared to administration of either the MVAΔE5R virus or of the immune checkpoint blocking agent, anti-cancer drug, or fingolimod (FTY720) alone.

In one aspect, the present disclosure provides a method of stimulating an immune response comprising administering to a subject an effective amount of the virus or the immunogenic composition. In some embodiments, the method further comprises separately, sequentially, or simultaneously administering to the subject one or more agents selected from: one or more immune checkpoint blocking agents; one or more anti-cancer drugs; fingolimod (FTY720); and any combination thereof. In some embodiments, the one or more immune checkpoint blocking agents is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof; and/or the one or more anti-cancer drugs is selected from the group consisting of a Mek inhibitor (U0126, selumitinib (AZD6244), PD98059, trametinib, cobimetinib), an EGFR inhibitor (lapatinib (LPN), erlotinib (ERL)), a HER2 inhibitor (lapatinib (LPN), Trastuzumab), a Raf inhibitor (sorafenib (SFN)), a BRAF inhibitor (dabrafenib, vemurafenib), an anti-OX40 antibody, a GITR agonist antibody, an anti-CSFR antibody, a CSFR inhibitor, paclitaxel, TLR9 agonist CpG, and a VEGF inhibitor (Bevacizumab), and any combination thereof. In some embodiments, the one or more immune checkpoint blocking agents comprises anti-PD-L1 antibody. In some embodiments, the one or more immune checkpoint blocking agents comprises anti-PD-1 antibody. In some embodiments, the one or more immune checkpoint blocking agents comprises anti-CTLA-4 antibody. In some embodiments, the combination of the MVAΔE5R virus with the immune checkpoint blocking agent, anti-cancer drug, and/or fingolimod (FTY720) has a synergistic effect in the stimulation of an immune response as compared to administration of the MVAΔE5R virus or of the immune checkpoint blocking agent, anti-cancer drug, or fingolimod (FTY720) alone.

In one aspect, the present disclosure provides a nucleic acid encoding the engineered MVAΔE5R viruses described herein.

In one aspect, the present disclosure provides a kit comprising the engineered MVAΔE5R viruses described herein, and instructions for use.

In one aspect, the present disclosure provides a vaccinia virus (VACV) genetically engineered to comprise a mutant E5R gene (VACVΔE5R). In some embodiments, the virus further comprises a heterologous nucleic acid molecule encoding one or more of OX40L, hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or a deletion of any one or more of thymidine kinase (TK), C7 (ΔC7L), E3L (ΔE3L), E3LΔ83N, B2R (ΔB2R), B19R (B18R; ΔWR200), IL18BP, K7R, C12L, B8R, B14R, N1L, C11R, K1L, M1L, N2L, or WR199. In some embodiments, the mutant E5R gene comprises replacement of at least a portion of the gene with one or more gene cassettes comprising the heterologous nucleic acid molecule. In some embodiments, the one or more gene cassettes comprises a heterologous nucleic acid molecule encoding OX40L (VACVΔE5R-OX40L). In some embodiments, the one or more gene cassettes further comprises a heterologous nucleic acid molecule encoding human Fms-like tyrosine kinase 3 ligand (hFlt3L) (VACVΔE5R-OX40L-hFlt3L). In some embodiments, the one or more gene cassettes comprises a heterologous nucleic acid molecule encoding human Fms-like tyrosine kinase 3 ligand (hFlt3L) (VACVΔE5R-hFlt3L). In some embodiments, the heterologous nucleic acid is expressed from within a viral gene selected from the group consisting of the thymidine kinase (TK) gene, the C7 gene, the C11 gene, the K3 gene, the F1 gene, the F2 gene, the F4 gene, the F6 gene, the F8 gene, the F9 gene, the F11 gene, the F14.5 gene, the J2 gene, the A46 gene, the E3L gene, the B18R gene (WR200), the E5R gene, the K7R gene, the C12L gene, the B8R gene, the B14R gene, the N1L gene, the K1L gene, the C16 gene, the M1L gene, the N2L gene, and the WR199 gene. In some embodiments, the virus further comprises a mutant thymidine kinase (TK) gene. In some embodiments, the mutant TK gene comprises replacement of at least a portion of the gene with one or more gene cassettes comprising a heterologous nucleic acid molecule. In some embodiments, the virus further comprises a mutant C7 gene. In some embodiments, the mutant C7 gene comprises an insertion of one or more gene cassettes comprising a heterologous nucleic acid molecule. In some embodiments, the mutant C7 gene comprises replacement of all or at least a portion of the gene with one or more gene cassettes comprising a heterologous nucleic acid molecule.

In one aspect, the present disclosure provides an immunogenic composition comprising the VACVΔE5R virus. In some embodiments, the immunogenic composition further comprises a pharmaceutically acceptable carrier. In some embodiments, the immunogenic composition further comprises a pharmaceutically acceptable adjuvant.

In one aspect, the present disclosure provides a method for treating a solid tumor in a subject in need thereof, the method comprising delivering to a tumor a composition comprising an effective amount of the VACVΔE5R virus or the immunogenic composition.

In some embodiments, the treatment comprises one or more of the following: inducing an immune response in the subject against the tumor or enhancing or promoting an ongoing immune response against the tumor in the subject, reducing the size of the tumor, eradicating the tumor, inhibiting the growth of the tumor, inhibiting metastatic growth of the tumor, inducing apoptosis of tumor cells, or prolonging survival of the subject. In some embodiments, the composition is administered by intratumoral or intravenous injection or a simultaneous or sequential combination of intratumoral and intravenous injection. In some embodiments, the tumor is melanoma, colon, breast, bladder, or prostate carcinoma. In some embodiments, the composition further comprises one or more agents selected from: one or more immune checkpoint blocking agents; one or more anti-cancer drugs; fingolimod (FTY720); and any combination thereof.

In some embodiments, the method further comprises separately, sequentially, or simultaneously administering to the subject one or more agents selected from: one or more immune checkpoint blocking agents; one or more anti-cancer drugs, fingolimod (FTY720); and any combination thereof. In some embodiments, the one or more immune checkpoint blocking agents is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof; and/or the one or more anti-cancer drugs is selected from the group consisting of a Mek inhibitor (U0126, selumitinib (AZD6244), PD98059, trametinib, cobimetinib), an EGFR inhibitor (lapatinib (LPN), erlotinib (ERL)), a HER2 inhibitor (lapatinib (LPN), Trastuzumab), a Raf inhibitor (sorafenib (SFN)), a BRAF inhibitor (dabrafenib, vemurafenib), an anti-OX40 antibody, a GITR agonist antibody, an anti-CSFR antibody, a CSFR inhibitor, paclitaxel, TLR9 agonist CpG, and a VEGF inhibitor (Bevacizumab), and any combination thereof. In some embodiments, the one or more immune checkpoint blocking agents comprises anti-PD-L1 antibody. In some embodiments, the one or more immune checkpoint blocking agents comprises anti-PD-1 antibody. In some embodiments, the one or more immune checkpoint blocking agents comprises anti-CTLA-4 antibody. In some embodiments, the combination of the VACVΔE5R virus with the immune checkpoint blocking agent, anti-cancer drug, and/or fingolimod (FTY720) has a synergistic effect in the treatment of the tumor as compared to administration of the VACVΔE5R virus or of the immune checkpoint blocking agent, anti-cancer drug, or fingolimod (FTY720) alone.

In one aspect, the present disclosure provides a method of stimulating an immune response comprising administering to a subject an effective amount of the virus or the immunogenic composition of. In some embodiments, the method further comprises separately, sequentially, or simultaneously administering to the subject one or more agents selected from: one or more immune checkpoint blocking agents; one or more anti-cancer drugs; fingolimod (FTY720); and any combination thereof. In some embodiments, the one or more immune checkpoint blocking agents is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof; and/or the one or more anti-cancer drugs is selected from the group consisting of a Mek inhibitor (U0126, selumitinib (AZD6244), PD98059, trametinib, cobimetinib), an EGFR inhibitor (lapatinib (LPN), erlotinib (ERL)), a HER2 inhibitor (lapatinib (LPN), Trastuzumab), a Raf inhibitor (sorafenib (SFN)), a BRAF inhibitor (dabrafenib, vemurafenib), an anti-OX40 antibody, a GITR agonist antibody, an anti-CSFR antibody, a CSFR inhibitor, paclitaxel, TLR9 agonist CpG, and a VEGF inhibitor (Bevacizumab), and any combination thereof. In some embodiments, the one or more immune checkpoint blocking agents comprises anti-PD-L1 antibody. In some embodiments, the one or more immune checkpoint blocking agents comprises anti-PD-1 antibody. In some embodiments, the one or more immune checkpoint blocking agents comprises anti-CTLA-4 antibody. In some embodiments, the combination of the VACVΔE5R virus with the immune checkpoint blocking agent, anti-cancer drug, and/or fingolimod (FTY720) has a synergistic effect in the stimulation of an immune response as compared to administration of the VACVΔE5R virus or of the immune checkpoint blocking agent, anti-cancer drug, or fingolimod (FTY720) alone.

In one aspect, the present disclosure provides a nucleic acid encoding the engineered VACVΔE5R viruses of the present technology.

In one aspect, the present disclosure provides a kit comprising the engineered VACVΔE5R viruses of the present technology, and instructions for use.

In one aspect, the present disclosure provides a myxoma virus (MYXV) genetically engineered to comprise a mutant M31R gene (MYXVΔM31R). In some embodiments, the virus further comprises a heterologous nucleic acid molecule encoding one or more of OX40L, hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or a deletion of any one or more of myxoma orthologs of vaccinia virus thymidine kinase (TK), C7 (ΔC7L), E3L (ΔE3L), E3LΔ83N, B2R (ΔB2R), B19R (B18R; ΔWR200), IL18BP, K7R, C12L, B8R, B14R, N1L, C11R, K1L, M1L, N2L, or WR199. In some embodiments, the mutant M31R gene comprises replacement of at least a portion of the gene with one or more gene cassettes comprising the heterologous nucleic acid molecule. In some embodiments, the one or more gene cassettes comprises a heterologous nucleic acid molecule encoding OX40L (MYXVΔM31R-OX40L). In some embodiments, the one or more gene cassettes further comprises a heterologous nucleic acid molecule encoding human Fms-like tyrosine kinase 3 ligand (hFlt3L) (MYXVΔM31R-OX40L-hFlt3L). In some embodiments, the one or more gene cassettes comprises a heterologous nucleic acid molecule encoding human Fms-like tyrosine kinase 3 ligand (hFlt3L) (MYXVΔM31R-hFlt3L). In some embodiments, the heterologous nucleic acid is expressed from within a myxoma ortholog of a vaccinia viral gene selected from the group consisting of the thymidine kinase (TK) gene, the C7 gene, the C11 gene, the K3 gene, the F1 gene, the F2 gene, the F4 gene, the F6 gene, the F8 gene, the F9 gene, the F11 gene, the F14.5 gene, the J2 gene, the A46 gene, the E3L gene, the B18R gene (WR200), the E5R gene, the K7R gene, the C12L gene, the B8R gene, the B14R gene, the N1L gene, the K1L gene, the C16 gene, the M1L gene, the N2L gene, and the WR199 gene. In some embodiments, the virus further comprises a mutant myxoma ortholog of vaccinia virus thymidine kinase (TK) gene. In some embodiments, the mutant TK gene comprises replacement of at least a portion of the gene with one or more gene cassettes comprising a heterologous nucleic acid molecule. In some embodiments, the virus further comprises a mutant myxoma ortholog of vaccinia virus C7 gene. In some embodiments, the mutant C7 gene comprises an insertion of one or more gene cassettes comprising a heterologous nucleic acid molecule. In some embodiments, the mutant C7 gene comprises replacement of all or at least a portion of the gene with one or more gene cassettes comprising a heterologous nucleic acid molecule.

In one aspect, the present disclosure provides an immunogenic composition comprising the MYXVΔM31R virus. In some embodiments, the immunogenic composition further comprises a pharmaceutically acceptable carrier. In some embodiments, the immunogenic composition further comprises a pharmaceutically acceptable adjuvant.

In one aspect, the present disclosure provides a method for treating a solid tumor in a subject in need thereof, the method comprising delivering to a tumor a composition comprising an effective amount of the MYXVΔM31R virus or the immunogenic composition. In some embodiments, the treatment comprises one or more of the following: inducing an immune response in the subject against the tumor or enhancing or promoting an ongoing immune response against the tumor in the subject, reducing the size of the tumor, eradicating the tumor, inhibiting the growth of the tumor, inhibiting metastatic growth of the tumor, inducing apoptosis of tumor cells, or prolonging survival of the subject. In some embodiments, the composition is administered by intratumoral or intravenous injection or a simultaneous or sequential combination of intratumoral and intravenous injection. In some embodiments, the tumor is melanoma, colon, breast, bladder, or prostate carcinoma.

In some embodiments, the composition further comprises one or more agents selected from: one or more immune checkpoint blocking agents; one or more anti-cancer drugs; fingolimod (FTY720); and any combination thereof. In some embodiments, the method further comprises separately, sequentially, or simultaneously administering to the subject one or more agents selected from: one or more immune checkpoint blocking agents; one or more anti-cancer drugs, fingolimod (FTY720); and any combination thereof. In some embodiments, the one or more immune checkpoint blocking agents is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof; and/or the one or more anti-cancer drugs is selected from the group consisting of a Mek inhibitor (U0126, selumitinib (AZD6244), PD98059, trametinib, cobimetinib), an EGFR inhibitor (lapatinib (LPN), erlotinib (ERL)), a HER2 inhibitor (lapatinib (LPN), Trastuzumab), a Raf inhibitor (sorafenib (SFN)), a BRAF inhibitor (dabrafenib, vemurafenib), an anti-OX40 antibody, a GITR agonist antibody, an anti-CSFR antibody, a CSFR inhibitor, paclitaxel, TLR9 agonist CpG, and a VEGF inhibitor (Bevacizumab), and any combination thereof. In some embodiments, the one or more immune checkpoint blocking agents comprises anti-PD-L1 antibody. In some embodiments, the one or more immune checkpoint blocking agents comprises anti-PD-1 antibody. In some embodiments, the one or more immune checkpoint blocking agents comprises anti-CTLA-4 antibody. In some embodiments, the combination of the MYXVΔM31R virus with the immune checkpoint blocking agent, anti-cancer drug, and/or fingolimod (FTY720) has a synergistic effect in the treatment of the tumor as compared to administration of either the MYXVΔM31R virus or of the immune checkpoint blocking agent, anti-cancer drug, or fingolimod (FTY720) alone.

In one aspect, the present disclosure provides a method of stimulating an immune response comprising administering to a subject an effective amount of the virus or the immunogenic composition of. In some embodiments, the method further comprises separately, sequentially, or simultaneously administering to the subject one or more agents selected from: one or more immune checkpoint blocking agents; one or more anti-cancer drugs; fingolimod (FTY720); and any combination thereof. In some embodiments, the one or more immune checkpoint blocking agents is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof; and/or the one or more anti-cancer drugs is selected from the group consisting of a Mek inhibitor (U0126, selumitinib (AZD6244), PD98059, trametinib, cobimetinib), an EGFR inhibitor (lapatinib (LPN), erlotinib (ERL)), a HER2 inhibitor (lapatinib (LPN), Trastuzumab), a Raf inhibitor (sorafenib (SFN)), a BRAF inhibitor (dabrafenib, vemurafenib), an anti-OX40 antibody, a GITR agonist antibody, an anti-CSFR antibody, a CSFR inhibitor, paclitaxel, TLR9 agonist CpG, and a VEGF inhibitor (Bevacizumab), and any combination thereof. In some embodiments, the one or more immune checkpoint blocking agents comprises anti-PD-L1 antibody. In some embodiments, the one or more immune checkpoint blocking agents comprises anti-PD-1 antibody. In some embodiments, the one or more immune checkpoint blocking agents comprises anti-CTLA-4 antibody. In some embodiments, the combination of the MYXVΔM31R virus with the immune checkpoint blocking agent, anti-cancer drug, and/or fingolimod (FTY720) has a synergistic effect in the stimulation of an immune response as compared to administration of the MYXVΔM31R virus or of the immune checkpoint blocking agent, anti-cancer drug, or fingolimod (FTY720) alone.

In one aspect, the present disclosure provides a nucleic acid encoding the engineered MYXVΔM31R viruses of the present technology.

In one aspect, the present disclosure provides a kit comprising the engineered MYXVΔM31R viruses of the present technology, and instructions for use.

In one aspect, the present disclosure a vaccinia virus (VACV) genetically engineered to comprise a mutant B2R gene (VACVΔB2R).

In some embodiments, the VACVΔB2R virus further comprises a heterologous nucleic acid molecule encoding one or more of OX40L, hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or a deletion of any one or more of thymidine kinase (TK), C7 (ΔC7L), E3L (ΔE3L), E3LΔ83N, B19R (B18R; ΔWR200), IL18BP, K7R, C12L, B8R, B14R, N1L, C11R, K1L, M1L, N2L, or WR199. In some embodiments, the VACVΔB2R virus is selected from one or more of VACVΔE3L83NΔB2R, VACVΔE5RΔB2R, VACVΔE3L83NΔE5RΔB2R, VACVΔE3L83N-ΔTK-anti-huCTLA-4-ΔE5R-hFlt3L-OX40L-hIL-12-ΔB2R. In some embodiments, the mutant B2R gene comprises replacement of at least a portion of the gene with one or more gene cassettes comprising the heterologous nucleic acid molecule. In some embodiments, the one or more gene cassettes comprises a heterologous nucleic acid molecule encoding OX40L (VACVΔB2R-OX40L). In some embodiments, the one or more gene cassettes further comprises a heterologous nucleic acid molecule encoding human Fms-like tyrosine kinase 3 ligand (hFlt3L) (VACVΔB2R-OX40L-hFlt3L). In some embodiments, the one or more gene cassettes comprise a heterologous nucleic acid molecule encoding human Fms-like tyrosine kinase 3 ligand (hFlt3L) (VACVΔB2R-hFlt3L). In some embodiments, the heterologous nucleic acid is expressed from within a viral gene selected from the group consisting of the thymidine kinase (TK) gene, the C7 gene, the C11 gene, the K3 gene, the F1 gene, the F2 gene, the F4 gene, the F6 gene, the F8 gene, the F9 gene, the F11 gene, the F14.5 gene, the J2 gene, the A46 gene, the E3L gene, the B18R (WR200) gene, the E5R gene, the K7R gene, the C12L gene, the B8R gene, the B14R gene, the N1L gene, the K1L gene, the C16 gene, the M1L gene, the N2L gene, and the WR199 gene.

In some embodiments, the present disclosure provides an immunogenic composition comprising the VACVΔB2R virus. In some embodiments, the immunogenic composition further comprises a pharmaceutically acceptable carrier. In some embodiments, the immunogenic composition further comprises a pharmaceutically acceptable adjuvant.

In some embodiments, the present disclosure provides a method for treating a solid tumor in a subject in need thereof, the method comprising delivering to a tumor a composition comprising an effective amount of the VACVΔB2R virus or the immunogenic composition.

In some embodiments, the treatment comprises one or more of the following: inducing an immune response in the subject against the tumor or enhancing or promoting an ongoing immune response against the tumor in the subject, reducing the size of the tumor, eradicating the tumor, inhibiting the growth of the tumor, inhibiting metastatic growth of the tumor, inducing apoptosis of tumor cells, or prolonging survival of the subject.

In some embodiments, the composition is administered by intratumoral or intravenous injection or a simultaneous or sequential combination of intratumoral and intravenous injection.

In some embodiments, the tumor is melanoma, colon, breast, bladder, or prostate carcinoma.

In some embodiments, the composition further comprises one or more agents selected from: one or more immune checkpoint blocking agents; one or more anti-cancer drugs; fingolimod (FTY720); and any combination thereof. In some embodiments, the method further comprises separately, sequentially, or simultaneously administering to the subject one or more agents selected from: one or more immune checkpoint blocking agents; one or more anti-cancer drugs, fingolimod (FTY720); and any combination thereof. In some embodiments, the one or more immune checkpoint blocking agents is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof; and/or the one or more anti-cancer drugs is selected from the group consisting of a Mek inhibitor (U0126, selumitinib (AZD6244), PD98059, trametinib, cobimetinib), an EGFR inhibitor (lapatinib (LPN), erlotinib (ERL)), a HER2 inhibitor (lapatinib (LPN), Trastuzumab), a Raf inhibitor (sorafenib (SFN)), a BRAF inhibitor (dabrafenib, vemurafenib), an anti-OX40 antibody, a GITR agonist antibody, an anti-CSFR antibody, a CSFR inhibitor, paclitaxel, TLR9 agonist CpG, and a VEGF inhibitor (Bevacizumab), and any combination thereof. In some embodiments, the one or more immune checkpoint blocking agents comprises anti-PD-L1 antibody. In some embodiments, the one or more immune checkpoint blocking agents comprises anti-PD-1 antibody. In some embodiments, the one or more immune checkpoint blocking agents comprises anti-CTLA-4 antibody. In some embodiments, in the combination of the VACVΔB2R virus with the immune checkpoint blocking agent, anti-cancer drug, and/or fingolimod (FTY720) has a synergistic effect in the treatment of the tumor as compared to administration of the VACVΔE5R virus or of the immune checkpoint blocking agent, anti-cancer drug, or fingolimod (FTY720) alone.

In some embodiments, the present disclosure provides a method of stimulating an immune response comprising administering to a subject an effective amount of the virus or the immunogenic composition. In some embodiments, the method further comprises separately, sequentially, or simultaneously administering to the subject one or more agents selected from: one or more immune checkpoint blocking agents; one or more anti-cancer drugs; fingolimod (FTY720); and any combination thereof. In some embodiments, the one or more immune checkpoint blocking agents is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof; and/or the one or more anti-cancer drugs is selected from the group consisting of a Mek inhibitor (U0126, selumitinib (AZD6244), PD98059, trametinib, cobimetinib), an EGFR inhibitor (lapatinib (LPN), erlotinib (ERL)), a HER2 inhibitor (lapatinib (LPN), Trastuzumab), a Raf inhibitor (sorafenib (SFN)), a BRAF inhibitor (dabrafenib, vemurafenib), an anti-OX40 antibody, a GITR agonist antibody, an anti-CSFR antibody, a CSFR inhibitor, paclitaxel, TLR9 agonist CpG, and a VEGF inhibitor (Bevacizumab), and any combination thereof. In some embodiments, the one or more immune checkpoint blocking agents comprises anti-PD-L1 antibody. In some embodiments, the one or more immune checkpoint blocking agents comprises anti-PD-1 antibody. In some embodiments, the one or more immune checkpoint blocking agents comprises anti-CTLA-4 antibody. In some embodiments, the combination of the VACVΔB2R virus with the immune checkpoint blocking agent, anti-cancer drug, and/or fingolimod (FTY720) has a synergistic effect in the stimulation of an immune response as compared to administration of the VACVΔB2R virus or of the immune checkpoint blocking agent, anti-cancer drug, or fingolimod (FTY720) alone.

In some embodiments, the present disclosure provides a nucleic acid encoding the VACVΔB2R virus of the present technology.

In some embodiments, the present disclosure provides a kit comprising the VACVΔB2R virus of the present technology, and instructions for use.

In one aspect, the present disclosure provides a myxoma virus (MYXV) genetically engineered to comprise one or more mutants selected from (i) a mutant M63R gene (MYXVΔM63R); (ii) a mutant M64R gene (MYXVΔM64R); and (iii) a mutant M62R gene (MYXVΔM62R). In some embodiments, the virus further comprises a heterologous nucleic acid molecule encoding one or more of OX40L, hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or a deletion of any one or more of myxoma orthologs of vaccinia virus thymidine kinase (TK), C7 (ΔC7L), E3L (ΔE3L), E3LΔ83N, B2R (ΔB2R), B19R (B18R; ΔWR200), IL18BP, K7R, C12L, B8R, B14R, N1L, C11R, K1L, M1L, N2L, or WR199 (ΔWR199), or of myxoma M31R (ΔM31R). In some embodiments, the mutant M63R gene, M64R gene, and/or M62R gene comprises replacement of at least a portion of the gene with one or more gene cassettes comprising the heterologous nucleic acid molecule. In some embodiments, the heterologous nucleic acid is expressed from within a myxoma ortholog of a vaccinia viral gene selected from the group consisting of the thymidine kinase (TK) gene, the C7 gene, the C11 gene, the K3 gene, the F1 gene, the F2 gene, the F4 gene, the F6 gene, the F8 gene, the F9 gene, the F11 gene, the F14.5 gene, the J2 gene, the A46 gene, the E3L gene, the B2R gene, the B18R (WR200) gene, the E5R gene, the K7R gene, the C12L gene, the B8R gene, the B14R gene, the N1L gene, the K1L gene, the C16 gene, the M1L gene, the N2L gene, and the WR199 gene.

In some embodiments, the present disclosure provides an immunogenic composition comprising the MYXV virus of the present technology. In some embodiments, the immunogenic composition further comprises a pharmaceutically acceptable carrier. In some embodiments, the immunogenic composition further comprises a pharmaceutically acceptable adjuvant.

In some embodiments, the present disclosure provides a method for treating a solid tumor in a subject in need thereof, the method comprising delivering to a tumor a composition comprising an effective amount of the MYXV virus of the present technology or the immunogenic composition. In some embodiments, the treatment comprises one or more of the following: inducing an immune response in the subject against the tumor or enhancing or promoting an ongoing immune response against the tumor in the subject, reducing the size of the tumor, eradicating the tumor, inhibiting the growth of the tumor, inhibiting metastatic growth of the tumor, inducing apoptosis of tumor cells, or prolonging survival of the subject.

In some embodiments, the composition is administered by intratumoral or intravenous injection or a simultaneous or sequential combination of intratumoral and intravenous injection. In some embodiments, the tumor is melanoma, colon, breast, bladder, or prostate carcinoma.

In some embodiments, the composition further comprises one or more agents selected from: one or more immune checkpoint blocking agents; one or more anti-cancer drugs; fingolimod (FTY720); and any combination thereof. In some embodiments, the method further comprises separately, sequentially, or simultaneously administering to the subject one or more agents selected from: one or more immune checkpoint blocking agents; one or more anti-cancer drugs, fingolimod (FTY720); and any combination thereof. In some embodiments, the one or more immune checkpoint blocking agents is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof; and/or the one or more anti-cancer drugs is selected from the group consisting of a Mek inhibitor (U0126, selumitinib (AZD6244), PD98059, trametinib, cobimetinib), an EGFR inhibitor (lapatinib (LPN), erlotinib (ERL)), a HER2 inhibitor (lapatinib (LPN), Trastuzumab), a Raf inhibitor (sorafenib (SFN)), a BRAF inhibitor (dabrafenib, vemurafenib), an anti-OX40 antibody, a GITR agonist antibody, an anti-CSFR antibody, a CSFR inhibitor, paclitaxel, TLR9 agonist CpG, and a VEGF inhibitor (Bevacizumab), and any combination thereof. In some embodiments, the one or more immune checkpoint blocking agents comprises anti-PD-L1 antibody. In some embodiments, the one or more immune checkpoint blocking agents comprises anti-PD-1 antibody. In some embodiments, the one or more immune checkpoint blocking agents comprises anti-CTLA-4 antibody. In some embodiments, the combination of the MYXVΔM62R, MYXVΔM63R, and/or MYXVΔM64R virus with the immune checkpoint blocking agent, anti-cancer drug, and/or fingolimod (FTY720) has a synergistic effect in the treatment of the tumor as compared to administration of either the MYXVΔM62R, MYXVΔM63R, and/or MYXVΔM64R virus or of the immune checkpoint blocking agent, anti-cancer drug, or fingolimod (FTY720) alone.

In some embodiments, the present disclosure provides a method of stimulating an immune response comprising administering to a subject an effective amount of the virus of or the immunogenic composition. In some embodiments, the method further comprises separately, sequentially, or simultaneously administering to the subject one or more agents selected from: one or more immune checkpoint blocking agents; one or more anti-cancer drugs; fingolimod (FTY720); and any combination thereof. In some embodiments, the one or more immune checkpoint blocking agents is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof; and/or the one or more anti-cancer drugs is selected from the group consisting of a Mek inhibitor (U0126, selumitinib (AZD6244), PD98059, trametinib, cobimetinib), an EGFR inhibitor (lapatinib (LPN), erlotinib (ERL)), a HER2 inhibitor (lapatinib (LPN), Trastuzumab), a Raf inhibitor (sorafenib (SFN)), a BRAF inhibitor (dabrafenib, vemurafenib), an anti-OX40 antibody, a GITR agonist antibody, an anti-CSFR antibody, a CSFR inhibitor, paclitaxel, TLR9 agonist CpG, and a VEGF inhibitor (Bevacizumab), and any combination thereof. In some embodiments, the one or more immune checkpoint blocking agents comprises anti-PD-L1 antibody. In some embodiments, the one or more immune checkpoint blocking agents comprises anti-PD-1 antibody. In some embodiments, the one or more immune checkpoint blocking agents comprises anti-CTLA-4 antibody. In some embodiments, the combination of the MYXV virus with the immune checkpoint blocking agent, anti-cancer drug, and/or fingolimod (FTY720) has a synergistic effect in the stimulation of an immune response as compared to administration of the MYXV virus or of the immune checkpoint blocking agent, anti-cancer drug, or fingolimod (FTY720) alone.

In some embodiments, the present disclosure provides a nucleic acid encoding the MYXV virus.

In some embodiments, the virus further comprises a heterologous nucleic acid molecule encoding hIL-12. In some embodiments, the virus comprises MVAΔE3LΔE5R-hFlt3L-OX40LΔWR199-hIL-12. In some embodiments, the virus further comprises a mutant C11R gene (MVAΔE3LΔE5R-hFlt3L-OX40LΔWR199-hIL-12ΔC11R). In some embodiments, the virus further comprises a nucleic acid molecule encoding hIL-15/IL-15Rα.

In some embodiments, the virus further comprises a mutant ΔE3L83N, a mutant thymidine kinase (ΔTK), a mutant B2R (ΔB2R), a mutant WR199 (ΔWR199), and a mutant WR200 (ΔSR200), and comprising a nucleic acid molecule encoding anti-CTLA-4 and a nucleic acid molecule encoding IL-12 (VACVΔE3L83N-ΔTK-anti-CTLA-4-ΔE5R-hFlt3L-OX40L-IL-12ΔB2RΔWR199ΔWR200). In some embodiments, the VACVΔE3L83N-ΔTK-anti-CTLA-4-ΔE5R-hFlt3L-OX40L-IL-12ΔB2RΔWR199ΔWR200 virus further comprises a nucleic acid molecule encoding hIL-15/IL-15Rα (VACVΔE3L83N-ΔTK-anti-CTLA-4-ΔE5R-hFlt3L-OX40L-IL-12ΔB2RΔWR199ΔWR200-hIL-15/IL-15Rα). In some embodiments, the VACVΔE3L83N-ΔTK-anti-CTLA-4-ΔE5R-hFlt3L-OX40L-IL-12ΔB2RΔWR199ΔWR200 virus further comprises a mutant C11R gene (ΔC11R) (VACVΔE3L83N-ΔTK-anti-CTLA-4-ΔE5R-hFlt3L-OX40L-IL-12ΔB2RΔWR199ΔWR200ΔC11R). In some embodiments, the VACVΔE3L83N-ΔTK-anti-CTLA-4-ΔE5R-hFlt3L-OX40L-IL-12ΔB2RΔWR199ΔWR200ΔC11R virus further comprises a nucleic acid molecule encoding hIL-15/IL-15Rα (VACVΔE3L83N-ΔTK-anti-CTLA-4-ΔE5R-hFlt3L-OX40L-IL-12ΔB2RΔWR199ΔWR200-hIL-15/IL-15Rα ΔC11R).

In some embodiments, MYXV viruses of the present technology are genetically engineered to comprise a mutant M62R gene (ΔM62R), a mutant M63R gene (ΔM63R), and a mutant M64R gene (ΔM64R) (MYXVΔM62RΔM63RΔM64R).

In one aspect, the present disclosure provides a recombinant poxvirus selected from the group consisting of: MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-CTLA-4, and MYXVΔM62RΔM63RΔM64RΔM31R-hFlt3L-OX40L-IL-12-IL-15/IL-15Rα-CTLA-4.

In some embodiments, the present disclosure provides a nucleic acid sequence encoding the recombinant poxvirus.

In some embodiments, the present disclosure provides a kit comprising the recombinant poxvirus.

In some embodiments, the present disclosure provides an immunogenic composition comprising the recombinant poxvirus. In some embodiments, the immunogenic composition further comprises a pharmaceutically acceptable carrier. In some embodiments, the immunogenic composition further comprises a pharmaceutically acceptable adjuvant.

In some embodiments, the present disclosure provides a method for treating a solid tumor in a subject in need thereof, the method comprising delivering to a tumor a composition comprising an effective amount of the recombinant poxvirus or the immunogenic composition. In some embodiments, the treatment comprises one or more of the following: inducing an immune response in the subject against the tumor or enhancing or promoting an ongoing immune response against the tumor in the subject, reducing the size of the tumor, eradicating the tumor, inhibiting the growth of the tumor, inhibiting metastatic growth of the tumor, inducing apoptosis of tumor cells, or prolonging survival of the subject.

In some embodiments, the composition is administered by intratumoral or intravenous injection or a simultaneous or sequential combination of intratumoral and intravenous injection.

In some embodiments, the tumor is melanoma, colon, breast, bladder, or prostate carcinoma.

In some embodiments, the composition further comprises one or more agents selected from: one or more immune checkpoint blocking agents; one or more anti-cancer drugs; fingolimod (FTY720); and any combination thereof. In some embodiments, the method further comprises separately, sequentially, or simultaneously administering to the subject one or more agents selected from: one or more immune checkpoint blocking agents; one or more anti-cancer drugs, fingolimod (FTY720); and any combination thereof. In some embodiments, the one or more immune checkpoint blocking agents is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof; and/or the one or more anti-cancer drugs is selected from the group consisting of a Mek inhibitor (U0126, selumitinib (AZD6244), PD98059, trametinib, cobimetinib), an EGFR inhibitor (lapatinib (LPN), erlotinib (ERL)), a HER2 inhibitor (lapatinib (LPN), Trastuzumab), a Raf inhibitor (sorafenib (SFN)), a BRAF inhibitor (dabrafenib, vemurafenib), an anti-OX40 antibody, a GITR agonist antibody, an anti-CSFR antibody, a CSFR inhibitor, paclitaxel, TLR9 agonist CpG, and a VEGF inhibitor (Bevacizumab), and any combination thereof. In some embodiments, the one or more immune checkpoint blocking agents comprises anti-PD-L1 antibody. In some embodiments, the one or more immune checkpoint blocking agents comprises. anti-PD-1 antibody. In some embodiments, the one or more immune checkpoint blocking agents comprises anti-CTLA-4 antibody. In some embodiments, the combination of the recombinant poxvirus with the immune checkpoint blocking agent, anti-cancer drug, and/or fingolimod (FTY720) has a synergistic effect in the treatment of the tumor as compared to administration of the recombinant poxvirus or of the immune checkpoint blocking agent, anti-cancer drug, or fingolimod (FTY720) alone.

In some embodiments, the present disclosure provides a method of stimulating an immune response comprising administering to a subject an effective amount of the virus or the immunogenic composition. In some embodiments, the method further comprises separately, sequentially, or simultaneously administering to the subject one or more agents selected from: one or more immune checkpoint blocking agents; one or more anti-cancer drugs; fingolimod (FTY720); and any combination thereof. In some embodiments, the one or more immune checkpoint blocking agents is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof; and/or the one or more anti-cancer drugs is selected from the group consisting of a Mek inhibitor (U0126, selumitinib (AZD6244), PD98059, trametinib, cobimetinib), an EGFR inhibitor (lapatinib (LPN), erlotinib (ERL)), a HER2 inhibitor (lapatinib (LPN), Trastuzumab), a Raf inhibitor (sorafenib (SFN)), a BRAF inhibitor (dabrafenib, vemurafenib), an anti-OX40 antibody, a GITR agonist antibody, an anti-CSFR antibody, a CSFR inhibitor, paclitaxel, TLR9 agonist CpG, and a VEGF inhibitor (Bevacizumab), and any combination thereof. In some embodiments, the one or more immune checkpoint blocking agents comprises anti-PD-L1 antibody. In some embodiments, the one or more immune checkpoint blocking agents comprises anti-PD-1 antibody. In some embodiments, the one or more immune checkpoint blocking agents comprises anti-CTLA-4 antibody. In some embodiments, the combination of the recombinant poxvirus with the immune checkpoint blocking agent, anti-cancer drug, and/or fingolimod (FTY720) has a synergistic effect in the stimulation of an immune response as compared to administration of the recombinant poxvirus or of the immune checkpoint blocking agent, anti-cancer drug, or fingolimod (FTY720) alone.

### BRIEF DESCRIPTION OF THE DRAWINGS

FIG. 1 is a schematic diagram of homologous recombination between plasmid DNA pCB vector and MVAΔE3L viral genomic DNA at the thymidine kinase gene (TK; J2R) locus. pCB-gpt plasmid was used to insert murine OX40L gene under the control of the vaccinia synthetic early and late promoter (PsE/L) into the TK locus. In this case, drug selection marker (gpt) is under the control of the vaccinia p7.5 promoter. The expression cassette was flanked by partial sequence of TK gene flank regions (TK-L and TK-R) on each side.

FIGS. 2A-2B show the verification of OX40L expression from recombinant virus MVAΔE3L-TK(−)-mOX40L. FIG. 2A is an image of PCR amplification of mOX40L gene and TK gene in MVAΔE3L and MVAΔE3L-TK(−)-mOX40L viral genome. FIG. 2B: Representative FACS plots showing the expression of mOX40L in B16-F10 cells infected with MVAΔE3L-TK(−)-mOX40L. Briefly, B16-F10 murine melanoma cells were infected at a MOI of 10 for 24 hours. Cells were then stained with PE-conjugated anti-mOX40L antibody.

FIGS. 3A-3H are a series of graphical representations of data showing that intratumoral injection of MVAΔE3L-OX40L generated more activated tumor-infiltrating effector T cells in distant tumors compared with MVAΔE3L in B16-F10 bilateral tumor model. B16-F10 murine melanoma bilateral tumor implantation model was used. Briefly, B16-F10 melanoma cells were implanted intradermally to the left and right flanks of C57B/6J mice (5×10⁵ to the right flank and 2.5×10⁵ to the left flank). Seven days post tumor implantation, 2×10⁷ pfu of either MVAΔE3L, MVAΔE3L-OX40L, or PBS was intratumorally (IT) injected into the larger tumors on the right flank twice, three days apart. Tumors were harvested at 2 days post second injection and tumor infiltrating lymphocytes were analyzed by FACS. FIGS. 3A-3C: Representative dot plots of Granzyme B⁺ CD8⁺ T cells in none-injected tumors after treatment with either MVAΔE3L, MVAΔE3L-OX40L, or PBS. FIG. 3D: Graph of percentages of Granzyme B⁺ CD8⁺ T cells out of CD8⁺ cells. Data are means±SEM (n=3 or 4). (**P<0.01; t test). FIGS. 3E-3G: Representative dot plots of Granzyme B⁺ CD4⁺ T cells in non-injected tumors after treatment with MVAΔE3L, MVAΔE3L-OX40L, or PBS. FIG. 3H: Graph of percentages of Granzyme B⁺ CD4⁺ T cells out of CD4⁺ cells. Data are means±SEM (n=3 or 4). (**P<0.01; ***P<0.001, t test).

FIGS. 4A and 4B are representative ELISPOT blots and graph showing that IT injection of MVAΔE3L-OX40L generated more antitumor CD8+ T cells in the spleens compared with MVA. B16-F10-bearing mice were treated with IT injection of either MVAΔE3L, MVAΔE3L-hFlt3L at 2×10⁷ pfu, or PBS twice, three days apart. Spleens were collected at 2 days after second injection. ELISPOT assay was performed by co-culturing irradiated B16-F10 cells (150,000) and purified CD8⁺ T cells (300,000) in a 96-well plate. FIG. 4A: Image of ELISPOT of triplicate samples from left to right. FIG. 4B: Graph of IFN-γ⁺ spots per 300,000 purified CD8⁺ T cells. Each bar represents spleen sample from individual mouse (n=3 or 4).

FIGS. 5A and 5B show a schematic diagram of two-step homologous recombination to generate MVAΔC7L-hFlt3L-TK(−)-muOX40L. FIG. 5A: First step: homologous recombination between plasmid DNA pUC57 vector and MVA viral genomic DNA at the C6 and C8 gene flanking C7 locus to insert hFlt3L and GFP expression cassette into the C7 locus (replacing C7 gene). The human Flt3L gene is under the control of the vaccinia synthetic early and late promoter (PsE/L). GFP is under the control of the vaccinia P7.5 promoter. FIG. 5B: Second step: homologous recombination between plasmid DNA pCB vector and MVAΔC7L-hFlt3L viral genomic DNA at the TK (J2R) gene locus to insert muOX40L and drug selection marker expression cassette into the TK (J2R) gene locus. The murine OX40L gene is under the control of the vaccinia synthetic early and late promoter (PsE/L). The drug selection marker gpt is under the control of the vaccinia P7.5 promoter. FIG. 5C shows that viral genomic DNAs were analyzed by PCR to verify the expression of OX40L and hFlt3L and confirm the insertion of the transgenes.

FIG. 6 are a series of dot plots from FACS analysis demonstrating hFlt3L expression in B16-F10 and SK-MEL-28 cell lines infected with either MVAΔC7L-hFlt3L or MVAΔC7L-hFlt3L-TK(−)-muOX40L. Cells were infected at a MOI of 10 for 24 hours prior to antibody staining and FACS analysis. MVAΔC7L, MVAΔC7L-hFlt3L or MVAΔC7L-hFlt3L-TK(−)-muOX40L-infected cells expressed GFP marker.

FIG. 7 are a series of dot plots from FACS analysis demonstrating murine OX40L expression in B16-F10 and SK-MEL-28 cell lines infected with MVAΔC7L-hFlt3L-TK(−)-muOX40L. Cells were infected at a MOI of 10 for 24 hours prior to antibody staining and FACS analysis.

FIGS. 8A-8C are a series of graphical representations of data showing that intratumoral injection of MVAΔC7L-hFlt3L-TK(−)-muOX40L generated more activated tumor-infiltrating effector CD8⁺ T cells in distant tumors compared with MVAΔC7L, MVAΔC7L-hFlt3L, or Heat-inactivated MVAΔC7L-hFlt3L in a B16-F10 bilateral murine melanoma model. Briefly, B16-F10 melanoma cells were implanted intradermally to the left and right flanks of C57B/6J mice (5×10⁵ to the right flank and 2.5×10⁵ to the left flank). Seven days post tumor implantation, intratumoral (IT) injections (2×10⁷ pfu) of either MVAΔC7L-hFlt3L-TK(−)-muOX40L, MVAΔC7L, MVAΔC7L-hFlt3L, or Heat-inactivated MVAΔC7L-hFlt3L were performed to the larger tumors on the right flank twice, three days apart. The non-injected distant tumors were harvested at 2 days post second injection and tumor-infiltrating lymphocytes were analyzed by FACS. FIG. 8A: Representative dot plots of Granzyme B⁺ CD8⁺ T cells in none-injected tumors after treatment with either PBS, MVAΔC7L, MVAΔC7L-hFlt3L, MVAΔC7L-hFlt3L-muOX40L, or Heat-iMVAΔC7L-hFlt3L. FIG. 8B: Graph of the absolute numbers of CD8⁺ T cells per gram of distant non-injected tumors. Data are means±SEM (n=4 or 5). (*P<0.05; **P<0.01; t test). FIG. 8C: Graph of the absolute numbers of Granzyme B⁺CD8⁺ T cells per gram of distant non-injected tumors. Data are means±SEM (n=4 or 5). (*P<0.05; **P<0.01; t test).

FIGS. 9A-9C are a series of graphical representations of data showing that intratumoral injection of MVAΔC7L-hFlt3L-TK(−)-muOX40L generated more activated tumor-infiltrating effector CD4⁺ T cells in distant tumors compared with MVAΔC7L, MVAΔC7L-hFlt3L, or Heat-inactivated MVAΔC7L-hFlt3L in a B16-F10 bilateral murine melanoma model. Briefly, B16-F10 melanoma cells were implanted intradermally to the left and right flanks of C57B/6J mice (5×10⁵ to the right flank and 2.5×10⁵ to the left flank). Seven days post tumor implantation, intratumoral (IT) injections (2×10⁷ pfu) of either MVAΔC7L-hFlt3L-TK(−)-muOX40L, MVAΔC7L, MVAΔC7L-hFlt3L, or Heat-inactivated MVAΔC7L-hFlt3L were performed to the larger tumors on the right flank twice, three days apart. The distant non-injected tumors were harvested at 2 days post second injection and tumor-infiltrating lymphocytes were analyzed by FACS. FIG. 9A: Representative dot plots of Granzyme B⁺ CD4⁺ T cells in none-injected tumors after treatment with either PBS, MVAΔC7L, MVAΔC7L-hFlt3L, MVAΔC7L-hFlt3L-muOX40L, or Heat-iMVAΔC7L-hFlt3L. FIG. 9B: Graph of the absolute numbers of CD4⁺ T cells per gram of distant non-injected tumors. Data are means±SEM (n=4 or 5). (*P<0.05; **P<0.01; t test). FIG. 9C: Graph of the absolute numbers of Granzyme B⁺ CD4⁺ T cells per gram of distant non-injected tumors. Data are means±SEM (n=4 or 5). (*P<0.05; **P<0.01; t test).

FIGS. 10A and 10B are representative ELISPOT blots and graph showing that IT injection of MVAΔC7L-hFlt3L-TK(−)-muOX40L generated stronger antitumor CD8⁺ T cell responses in the spleens compared with MVAΔC7L, MVAΔC7L-hFlt3L, or Heat-inactivated MVAΔC7L-hFlt3L. B16-F10-bearing mice were treated with IT injection of either MVAΔC7L-hFlt3L-TK(−)-muOX40L, MVAΔC7L, MVAΔC7L-hFlt3L at 2×10⁷ pfu, or Heat-inactivated MVAΔC7L-hFlt3L twice, three days apart. Spleens were collected at 2 days after second injection. ELISPOT assay was performed by co-culturing irradiated B16-F10 cells (150,000) and purified CD8⁺ T cells (300,000) in a 96-well plate. FIG. 10A: Image of ELISPOT of triplicate samples from left to right. FIG. 10B: Graph of IFN-γ⁺ spots per 300,000 purified CD8⁺ T cells. Each bar represents spleen sample from individual mouse (n=5).

FIGS. 11A-11G are graphical representations of data showing the combination of IT MVAΔC7L-hFlt3L-TK(−)-muOX40L and systemic delivery immune checkpoint blockade antibody anti-CTLA-4 or anti-PD-L1 delays tumor growth and prolongs survival in murine B16-F10 melanoma bilateral tumor implantation model. FIG. 11A is a scheme of tumor implantation and treatment for a B16-F10 bilateral tumor implantation model. Briefly, B16-F10 melanoma cells were implanted intradermally to the left and right flanks of C57B/6J mice (5×10⁵ to the right flank and 1×10⁵ to the left flank). Nine days post tumor implantation, intratumoral injections (2×10⁷ pfu) of MVAΔC7L-hFlt3L-TK(−)mOX40L were performed twice weekly to the larger tumors on the right flank. Anti-CTLA-4 or anti-PD-L1 antibody at 250 μg per mouse was given intraperitoneally. The tumor sizes were measured and the survival of mice was monitored. FIGS. 11B and 11C are graphical representations of data showing volumes of injected (FIG. 11B) and non-injected (FIG. 11C) tumors over days after PBS, MVAΔC7L-hFlt3L-TK(−)-mOX40L, MVAΔC7L-hFlt3L-TK(−)-mOX40L plus anti-CTLA-4 antibody, MVAΔC7L-hFlt3L-TK(−)-mOX40L plus anti-PD-L1 antibody treatments. FIGS. 11D and 11E are graphical representations of data showing initial volumes of injected (FIG. 11D) and non-injected (FIG. 11E) tumors and at Day 7 and Day 11 post PBS, MVAΔC7L-hFlt3L-TK(−)-mOX40L, MVAΔC7L-hFlt3L-TK(−)-mOX40L plus anti-CTLA-4 antibody, MVAΔC7L-hFlt3L-TK(−)-mOX40L plus anti-PD-L1 antibody treatments. FIG. 11F is a graph of the Kaplan-Meier survival curve of tumor-bearing mice treated with either PBS, MVAΔC7L-hFlt3L-TK(−)-mOX40L, MVAΔC7L-hFlt3L-TK(−)-mOX40L plus anti-CTLA-4 antibody, MVAΔC7L-hFlt3L-TK(−)-mOX40L plus anti-PD-L1 antibody treatments. (n=10, **P<0.01; ***P<0.001; Mantel-Cox test). FIG. 11G is a table showing median survival of mice treated with either PBS, MVAΔC7L-hFlt3L-TK(−)-mOX40L, MVAΔC7L-hFlt3L-TK(−)-mOX40L plus anti-CTLA-4 antibody, MVAΔC7L-hFlt3L-TK(−)-mOX40L plus anti-PD-L1 antibody.

FIG. 12 are a series of graphical representations of data showing tumor growth curves in mice treated in a B16-F10 unilateral large tumor model. Briefly, B16-F10 melanoma cells were implanted intradermally to the right flank of C57B/6J mice (5×10⁵ cells). Eleven days post implantation viruses were injected intratumorally twice per week, and Anti-CTLA-4 or anti-PD-L1 antibodies were injected twice per week intraperitoneally.

FIGS. 13A and 13B are schematic diagrams of two-step homologous recombination to generate MVAΔC7L-hFlt3L-TK(−)-huOX40L. FIG. 13A: First step: homologous recombination between plasmid DNA pUC57 vector and MVA viral genomic DNA at the C6 and C8 gene flanking C7 locus to insert hFlt3L and GFP expression cassette into the C7 locus (replacing C7 gene). The human Flt3L gene is under the control of the vaccinia synthetic early and late promoter (PsE/L). GFP is under the control of the vaccinia P7.5 promoter. FIG. 13B: Second step: homologous recombination between plasmid DNA pUC57ΔTK-hOX40L-mCherry and MVAΔC7L-hFlt3L viral genomic DNA at the J1R and J3R (TK-R and TK-L) loci flanking J2R (TK) gene to insert huOX40L and mCherry expression cassette into the TK (J2R) gene locus. The human OX40L gene is under the control of the vaccinia synthetic early and late promoter (PsE/L). mCherry is under the control of the vaccinia P7.5 promoter. FIG. 13C: PCR verification of three independent clones of recombinant MVAΔC7L-hFlt3L-TK(−)-huOX40L, which contains hOX40L gene and hFlt3L gene insert but lacks TK (J2R) gene. H1, h2 and H3 are individual recombinant MVA. “+”: positive control for the PCR reaction.

FIGS. 14A and 14B are two graphs showing a multi-step growth of the parental MVA and recombinant viruses, including MVAΔC7L-hFlt3L, MVAΔC7L-hFlt3L-TK(−)-muOX40L, MVAΔC7L-hFlt3L-TK(−)-hOX40L in primary chicken embryo fibroblasts (CEFs). FIG. 14A is a multi-step growth curve of these viruses in CEFs. Briefly, CEFs were infected with the above-mentioned viruses at a MOI of 0.05. Cells were collected at 1, 24, 48 and 72 h. Viral titers were determined on BHK21 cells by serial dilution and counting GFP⁺ foci under confocal microscope. FIG. 14B show the log (fold change) of viral titers at 72 h post infection over 1 h post infection.

FIGS. 15A and 15B are a series of representative dot plots of FACS data showing the expression of hOX40L in BHK21 cells and human monocyte-derived dendritic cells (moDCs) infected by MVAΔC7L-hFlt3L-TK(−)-hOX40L virus. FIG. 15A: BHK21 cells were either mock-infected, or infected with MVAΔC7L-hFlt3L or with MVAΔC7L-hFlt3L-TK(−)-hOX40L at a MOI of 10. Cells were collected at 24 h post infection and stained with PE-conjugated anti-hOX40L antibody prior to FACS analyses. FIG. 15B: human moDCs were either treated with poly I:C at 10 μg/ml, or infected with Heat-iMVA, MVAΔC7L-TK(−), or MVAΔC7L-hFlt3L-TK(−)-hOX40L at MOI of 1. At 24 h post infection, cells were collected and stained with PE-conjugated anti-hOX40L antibody prior to FACS analyses. Untreated murine B16-F10 melanoma cells were used as a negative control.

FIGS. 16A and 16B are a series of graphical representations of data showing hOX40L mRNA levels in MVAΔC7L-hFlt3L-TK(−)-hOX40L-infected BHK21 (FIG. 16A) and B16-F10 cells (FIG. 16B). BHK21 or B16-F10 cells were infected with either MVA or MVAΔC7L-hFlt3L-TK(−)-hOX40L at a MOI of 10. At 8 and 16 h post infection, cells were collected and RNAs were extracted. Quantitative RT-PCR analyses were performed to examine the expression of viral E3L gene and hOX40L gene.

FIG. 17 is a schematic diagram showing the workflow of constructing vaccinia virus viral early gene expression plasmids. 72 viral early genes were selected. PCR was performed to amplify the gene of interest from vaccinia viral genome. Adaptors were added to both ends of PCR products by a second round of PCR. Then the DNA fragments were cloned into pDONR™/ZEO, and then to pcDNA™3.2-DEST, a mammalian expression vector. The DNA constructs were later verified by sequencing. The plasmid DNAs were then used to transfect into HEK-293T cells, along with other plasmids, which will be described below.

FIG. 18 shows dual luciferase screening strategy. In HEK293T cells, cGAS and STING expression plasmids were co-transfected with IFN-β luciferase plasmid and pRL-TK. Viral gene expression plasmids or vector were transfected together. After 24 h, luciferase signal was measured. The relative luciferase activity was expressed as arbitrary units by normalizing firefly luciferase activity to Renilla luciferase activity.

FIGS. 19A-19C show the dual luciferase screening results of vaccinia virus ORFs that inhibit cGAS/STING-dependent IFNβ-luc activity. FIGS. 19A-19C: HEK293T cells were transfected with plasmids expressing IFNβ-luc reporter, murine cGAS, human STING and vaccinia virus ORFs as indicated. Dual luciferase assays were performed 24 h after transfection. Adenovirus E1A gene was used as a positive control.

FIGS. 20A-20E. Vaccinia virus B18, E5, K7, C11 and B14 inhibits cGAS/STING-induced IFNβ promoter activity. HEK293T cells were transfected with IFNB luciferase reporter, cGAS, STING and expression plasmids as indicated, and luciferase activity was assayed 24 h after transfection. FIG. 20A: Mouse cGAS was co-transfected with expression plasmids. FIG. 20B: Human cGAS was co-transfected with expression plasmids. FIG. 20C: Mouse cGAS was co-transfected with FLAG-tagged vaccinia ORFs of K7R, E5R, B14R, C11R, and B18R. FIGS. 20D and 20E are charts showing the induction of IFNB in cells over-expressing E5, B14, K7, or B18 by Heat-iMVA infection (FIG. 20D) or ISD treatment (FIG. 20E).

FIG. 21 shows additional dual luciferase screening results of vaccinia virus ORFs that inhibit cGAS/STING-dependent IFNβ-luc activity. HEK293T cells were transfected with plasmids expressing IFNβ-luc reporter, murine cGAS, human STING and vaccinia virus ORFs as indicated. Dual luciferase assays were performed 24 h after transfection. Adenovirus E1A gene was used as a positive control.

FIG. 22 shows MVA genome sequence as set forth in SEQ ID NO: 1, and given by GenBank Accession No. U94848.1.

FIG. 23 shows the vaccinia virus (Western Reserve strain; WR) genome sequence as set forth in SEQ ID NO: 2, and given by GenBank Accession No. AY243312.1.

FIGS. 24A and 24B are two graphs showing a multi-step growth of the vaccinia and the recombinant viruses, including E3LΔ83N-TK⁻-hFlt3L-anti-muCTLA-4, E3LΔ83N-TK⁻-hFlt3L-anti-muCTLA-4/C7L⁻-mOX40L, and VAC-TK⁻-anti-muCTLA-4/C7L⁻-mOX40L in murine B16-F10 melanoma cells. FIG. 24A is a multi-step growth of these viruses in B16-F10 cells. Briefly, B16-F10 cells were infected with the above-mentioned viruses at a MOI of 0.1. Cells were collected at 1, 24, 48, and 72 h post infection and viral yields (log pfu) were determined by titrating on BSC40 cells. Viral yields were plotted against hours post infection. FIG. 24B shows the log (fold change) of viral titers at 72 h post infection over 1 h post infection.

FIG. 25 shows a Western blot analysis of anti-mCTLA-4 antibody, murine OX40L, and human Flt3L expression in E3LΔ83N-TK⁻-hFlt3L-anti-muCTLA-4 or E3LΔ83N-TK⁻-hFlt3L-anti-muCTLA-4/C7L⁻-mOX40L virus-infected murine B16-F10 melanoma cells. B16-F10 cells were infected or mock infected with E3LΔ83N-TK⁻-hFlt3L-anti-muCTLA-4 or E3LΔ83N-TK⁻-hFlt3L-anti-muCTLA-4/C7L⁻-mOX40L viruses at a MOI of 10. Cell lysates were collected at 7, 24 and 48 h post infection, and the polypeptides in cell lysates were separated using 10% SDS-PAGE. HRP-conjugated anti-mouse IgG (heavy and light chain), anti-mOX40L antibody, and anti-human Flt3L antibody was used to detect the anti-mCTLA-4 antibody, murine OX40L, and human Flt3L protein respectively.

FIG. 26 shows the surface expression of murine OX40L protein in E3LΔ83N-TK⁻-hFlt3L-anti-muCTLA-4/C7L⁻-mOX40L or VAC-TK⁻-anti-mCTLA-4/C7L⁻-mOX40L virus infected murine B16-F10 melanoma cells. Briefly, B16-F10 cells were infected or mock infected with E3LΔ83N-TK⁻-vector, E3LΔ83N-TK⁻-hFlt3L-anti-muCTLA-4/C7L⁻-vector, E3LΔ83N-TK⁻-hFlt3L-anti-muCTLA-4/C7L⁻-mOX40L, or VAC-TK⁻-anti-mCTLA-4/C7L⁻-mOX40L viruses at a MOI of 5. Cells were collected at 24 h post infection, and stained with PE-conjugated anti-mOX40L antibody, and analyzed by FACS. Data were analyzed with FlowJo software (FlowJo, Becton-Dickinson, Franklin Lakes, NJ).

FIG. 27 shows a scheme of tumor implantation and treatment for a B16-F10 murine melanoma unilateral tumor implantation model. Briefly, 5×10⁵ B16-F10 melanoma cells were implanted intradermally to the right flank of C57B/6J mice. Nine days post tumor implantation, 4×10⁷ pfu of MVAΔC7L-hFlt3L-TK(−)mOX40L were intratumorally injected twice weekly. Anti-PD-L1 antibody at 250 μg per mouse was given intraperitoneally. The tumor sizes were measured and the survival of mice was monitored.

FIGS. 28A-28C are graphical representations of data showing volumes of tumors over days after PBS (FIG. 28A), MVAΔC7L-hFlt3L-TK(−)-mOX40L (FIG. 28B), or MVAΔC7L-hFlt3L-TK(−)-mOX40L plus anti-PD-L1 antibody (FIG. 28C) treatments.

FIGS. 29A and 29B demonstrate survival studies of mice treated with either PBS, MVAΔC7L-hFlt3L-TK(−)-mOX40L, or MVAΔC7L-hFlt3L-TK(−)-mOX40L plus anti-PD-L1 antibody. FIG. 29A is a graph of the Kaplan-Meier survival curve of tumor-bearing mice treated with either PBS, MVAΔC7L-hFlt3L-TK(−)-mOX40L, or MVAΔC7L-hFlt3L-TK(−)-mOX40L plus anti-PD-L1 antibody treatments. (n=5˜10, **P<0.01; ***P<0.001; Mantel-Cox test). FIG. 29B is a table showing median survival of mice treated with either PBS, MVAΔC7L-hFlt3L-TK(−)-mOX40L, MVAΔC7L-hFlt3L-TK(−)-mOX40L plus anti-PD-L1 antibody.

FIG. 30 shows a scheme of tumor implantation and treatment for a MC38 unilateral tumor implantation model. Briefly, 5×10⁵ MC38 melanoma cells were implanted intradermally to the right flank of C57B/6J mice. Nine days post tumor implantation, 4×10⁷ pfu of MVAΔC7L-hFlt3L-TK(−)mOX40L were intratumorally injected twice weekly. Anti-PD-L1 antibody at 250 μg per mouse was given intraperitoneally. The tumor sizes were measured and the survival of mice was monitored.

FIGS. 31A-31C are graphical representations of data showing volumes of tumors over days after PBS (FIG. 31A), MVAΔC7L-hFlt3L-TK(−)-mOX40L (FIG. 31B), or MVAΔC7L-hFlt3L-TK(−)-mOX40L plus anti-PD-L1 antibody (FIG. 31C) treatments.

FIGS. 32A and 32B demonstrate survival studies of mice treated with either PBS, MVAΔC7L-hFlt3L-TK(−)-mOX40L, or MVAΔC7L-hFlt3L-TK(−)-mOX40L plus anti-PD-L1 antibody. FIG. 32A is a graph of the Kaplan-Meier survival curve of tumor-bearing mice treated with either PBS, MVAΔC7L-hFlt3L-TK(−)-mOX40L, or MVAΔC7L-hFlt3L-TK(−)-mOX40L plus anti-PD-L1 antibody treatments. (n=4-8, **P<0.01; ***P<0.001; Mantel-Cox test). FIG. 32B is a table showing median survival of mice treated with either PBS, MVAΔC7L-hFlt3L-TK(−)-mOX40L, or MVAΔC7L-hFlt3L-TK(−)-mOX40L plus anti-PD-L1 antibody.

FIG. 33 shows a scheme of tumor implantation and treatment for a MB49 unilateral tumor implantation model. Briefly, 2.5×10⁵ MB49 melanoma cells were implanted intradermally to the right flank of C57B/6J mice. Eight days post tumor implantation, 4×10⁷ pfu of MVAΔC7L-hFlt3L-TK(−)mOX40L were intratumorally injected twice weekly. Anti-PD-L1 antibody at 250 μg per mouse was given intraperitoneally. The tumor sizes were measured and the survival of mice was monitored.

FIGS. 34A-34D are graphical representations of data showing volumes of tumors over days after PBS (FIG. 34A), MVAΔC7L-hFlt3L-TK(−)-mOX40L (FIG. 34B), MVAΔC7L-hFlt3L-TK(−)-mOX40L plus anti-PD-L1 antibody (FIG. 34V), or anti-PD-L1 antibody (FIG. 34D) treatments.

FIGS. 35A and 35B demonstrate survival studies of mice treated with either PBS, MVAΔC7L-hFlt3L-TK(−)-mOX40L, MVAΔC7L-hFlt3L-TK(−)-mOX40L plus anti-PD-L1 antibody, or anti-PD-L1 antibody treatments. FIG. 35A is a graph of the Kaplan-Meier survival curve of tumor-bearing mice treated with either PBS, MVAΔC7L-hFlt3L-TK(−)-mOX40L, MVAΔC7L-hFlt3L-TK(−)-mOX40L plus anti-PD-L1 antibody, or anti-PD-L1 antibody treatments. (n=10, *P<0.05, **P<0.01; ***P<0.001; Mantel-Cox test). FIG. 35B is a table showing median survival of mice treated with either PBS, MVAΔC7L-hFlt3L-TK(−)-mOX40L, MVAΔC7L-hFlt3L-TK(−)-mOX40L plus anti-PD-L1 antibody, or anti-PD-L1 antibody.

FIG. 36 shows a representative graph of tumors isolated from a female MMTV-PyVmT mouse. Briefly, the mice were treated with IT injection of PBS, 4×10⁷ pfu of MVAΔC7L-hFlt3L-TK(−)-mOX40L twice weekly after developing palpable mammary tumors with a mean latency of 92 days of age. Anti-PD-L1 antibody at 250 μg per mouse was given intraperitoneally twice weekly. The tumor sizes were measured and the survival of mice was monitored.

FIG. 37 are graphical representations of data showing volumes of tumors over days after PBS, MVAΔC7L-hFlt3L-TK(−)-mOX40L, or MVAΔC7L-hFlt3L-TK(−)-mOX40L plus anti-PD-L1 antibody treatments. (P0—PBS; V1-MVAΔC7L-hFlt3L-muOX40L; C1, C2, C3—MVAΔC7L-hFlt3L-muOX40L+anti-PD-L1).

FIG. 38 shows representative dot plots of CD8⁺ and CD4⁺ T cells in injected and non-injected tumors after treatment with either PBS, MVAΔC7L-hFlt3L-TK(−)-muOX40L, or MVAΔC7L-hFlt3L-TK(−)-muOX40L plus anti-PD-L1 antibody.

FIG. 39 shows representative dot plots of CD8⁺CD69⁺CD103⁺ T cells in injected and non-injected tumors after treatment with either PBS, MVAΔC7L-hFlt3L-TK(−)-muOX40L, or MVAΔC7L-hFlt3L-TK(−)-muOX40L plus anti-PD-L1 antibody.

FIG. 40 shows representative dot plots of CD4⁺CD69⁺CD103⁺ T cells in injected and non-injected tumors after treatment with either PBS, MVAΔC7L-hFlt3L-TK(−)-muOX40L, or MVAΔC7L-hFlt3L-TK(−)-muOX40L plus anti-PD-L1 antibody.

FIGS. 41A and 41B are representative FACS plots showing the expression of hFlt3L (FIG. 41A) or mOX40L (FIG. 41B) by B16-F10-hFlt3L or B16-F10-mOX40L stable cell lines.

FIG. 42 is a scheme of tumor implantation and treatment for a B16-F10 bilateral tumor implantation model. Briefly, 5×10⁵ B16-F10 melanoma cells were implanted intradermally to right flanks of C57B/6J mice and 5×10⁵ B16-F10-hFlt3L melanoma cells were implanted intradermally to left flanks of C57B/6J mice. Nine days post tumor implantation, PBS or 4×10⁷ pfu of MVAΔC7L were intratumorally injected twice weekly to the tumors on both flanks. Tumors were harvested 2 days post second injection and tumor infiltrating lymphocytes (TILs) were analyzed by FACS.

FIGS. 43A-43D are graphical representations of data showing volumes of tumors in either the right of left flanks of C57B/6J mice over days after PBS or MVAΔC7L treatments.

FIGS. 44A-44E are graphs of the percentage of tumor infiltrating CD8⁺ (FIG. 44A), CD8⁺GranzymeB⁺ (FIG. 44B), CD4⁺ (FIG. 44C), CD4⁺GranzymeB⁺ (FIG. 44D), and CD4⁺FoxP3⁺ (FIG. 44E) T cells after PBS or MVAΔC7L treatments. (n=5, *P<0.05; **P<0.01; ***P<0.001, ****P<0.0001; One-way ANOVA).

FIGS. 45A-45E are graphs of the absolute numbers of tumor infiltrating CD8⁺ (FIG. 45A), CD8⁺GranzymeB⁺ (FIG. 45B), CD4⁺ (FIG. 45C), CD4⁺GranzymeB⁺ (FIG. 45D), CD4⁺FoxP3⁺ (FIG. 45E) T cells per gram of tumors after PBS, MVAΔC7L treatments. (n=5, *P<0.05; **P<0.01; ***P<0.001, ****P<0.0001; One-way ANOVA).

FIG. 46 is a scheme of tumor implantation and treatment for a B16-F10 bilateral tumor implantation model. Briefly, 5×10⁵ B16-F10 melanoma cells were implanted intradermally to right flanks of C57B/6J mice and 5×10⁵ B16-F10-OX40L melanoma cells were implanted intradermally to left flanks of C57B/6J mice. Nine days post tumor implantation, PBS or 4×10⁷ pfu of MVAΔC7L were intratumorally injected twice weekly to the tumors on both flanks. Tumors were harvested 2 days post second injection and tumor infiltrating lymphocytes (TILs) were analyzed by FACS.

FIGS. 47A-47D are graphical representations of data showing volumes of tumors in either the right of left flanks of C57B/6J mice over days after PBS or MVAΔC7L treatments.

FIGS. 48A-48E are graphs of the percentage of tumor infiltrating CD8⁺ (FIG. 48A), CD8⁺GranzymeB⁺ (FIG. 48B), CD4⁺ (FIG. 48C), CD4⁺GranzymeB⁺ (FIG. 48D), CD4⁺FoxP3⁺ (FIG. 48E) T cells after PBS, MVAΔC7L treatments. (n=5, *P<0.05; **P<0.01; ***P<0.001, ****P<0.0001; One-way ANOVA).

FIGS. 49A-49E are graphs of the absolute numbers of tumor infiltrating CD8⁺ (FIG. 49A), CD8⁺GranzymeB⁺ (FIG. 49B), CD4⁺ (FIG. 49C), CD4⁺GranzymeB⁺ (FIG. 49D), CD4⁺FoxP3⁺ (FIG. 49E) T cells per gram of tumors after PBS, MVAΔC7L treatments. (n=5, *P<0.05; **P<0.01; ***P<0.001, ****P<0.0001; One-way ANOVA).

FIGS. 50A and 50B show the mechanism of action of FTY720 and its chemical structure. FIG. 50A is adapted from a drawing in Gong et al. Front. Immunol. (2014), Naïve T cells circulate between lymphoid organs and blood. Upon infection or tumor implantation, antigen-presenting cells present antigen to prime cognate T cells, which then proliferate and differentiate into effector T cells and memory T cells. Effector T cells are recruited to the site of infection or tumors and memory T cells recirculate. FTY720 (FIG. 50B), a sphingosine-1-phosphate receptor modulator, blocks the exit of lymphocytes from lymphoid organs.

FIG. 51 is a scheme of tumor implantation and treatment for a B16-F10 unilateral tumor implantation model. Briefly, 5×10⁵ B16-F10 melanoma cells were implanted intradermally to right flanks of C57B/6J mice. Nine days post tumor implantation, PBS or 4×10⁷ pfu of MVAΔC7L-hFlt3L-TK(−)-muOX40L were intratumorally injected twice. FTY720 at 25 μg per mouse was given intraperitoneally daily during the treatment, starting 1 day prior to the first MVAΔC7L-hFlt3L-TK(−)-muOX40L injection. The tumor sizes were measured and the survival of mice was monitored.

FIGS. 52A-52D are graphical representations of data showing volumes of tumors in C57B/6J mice over days after PBS or MVAΔC7L treatments with or without FTY720 treatment.

FIGS. 53A and 53B are graphical representations of data showing volumes of tumors in C57B/6J mice over days after PBS or MVAΔC7L treatments with or without FTY720 treatment.

FIGS. 53C and 53D are graphs of the Kaplan-Meier survival curve of tumor-bearing mice treated with PBS or MVAΔC7L-hFlt3L-TK(−)-mOX40L with or without FTY720 (n=5˜10, **P<0.01; ***P<0.001; Mantel-Cox test).

FIGS. 54A and 54B show domain organization and sequence conservation of vaccinia E5 amongst the poxvirus family. FIG. 54A is a schematic diagram of vaccinia E5. E5 is 328-aa protein, which is comprised of a N-terminal domain followed by two BEN domains. BEN is named after its presence in BANP/SMAR1, poxvirus E5R, and NACL. BEN domain containing proteins are involved in chromatin organization, transcription regulation, and possibly viral DNA organization. FIG. 54B is a schematic diagram that demonstrates that vaccinia E5 is highly conserved in the poxvirus family.

FIGS. 55A-55C show that VACVΔE5R is highly attenuated in an intranasal infection model. FIG. 55A shows a scheme for generating VACVΔE5R virus through homologous recombination at the E4L and E6R loci flanking E5R gene of the vaccinia genome. FIG. 55B shows weight loss over days after intranasal infection with either WT VACV (2×10⁶ pfu), VACVΔE5R (2×10⁶ pfu), or VACVΔE5R (2×10⁷ pfu). FIG. 55C shows Kaplan-Meier survival curves of mice infected with WT VACV (2×10⁶ pfu) or VACVΔE5R (2×10⁶ pfu or 2×10⁷ pfu).

FIGS. 56A and 56B demonstrates that infection with VACVΔE5R of BMDCs induce IFNB gene expression and IFN-β protein secretion. FIG. 56A shows RT-PCR results of BMDCs that were infected MVA, VACV, or VACVΔE5R at a MOI of 10. Cells were collected at 8 h post infection. RNAs were extracted and RT-PCRs were performed. FIG. 56B shows that BMDCs were infected MVA, VACV, or VACVΔE5R at a MOI of 10. Supernatants were collected at 21 h post infection. IFN-β protein levels were determined by ELISA.

FIGS. 57A-57D show IFNBgene induction by MVAΔE5R and MVAΔK7R in BMDCs and BMDMs. FIG. 57A shows a scheme for generating MVAΔE5R virus through homologous recombination at the E4L and E6R loci flanking E5R gene of the MVA genome. FIG. 57B shows a scheme for generating MVAΔK7R virus through homologous recombination at the K5,6L and F1L loci flanking K7R gene of the MVA genome. FIG. 57C show that BMDCs were infected with either MVA, MVAΔE5R, or MVAΔK7R at MOI of 10. Cells were collected at 6 h post infection. IFNB gene expression was measured by RT-PCR. FIG. 57D shows that BMDMs were infected with either MVA, MVAΔE5R, or MVAΔK7R at MOI of 10. Cells were collected at 6 h post infection. IFNB gene expression was measured by RT-PCR.

FIGS. 58A-58C show that BMDCs were infected with either MVA or MVAΔE5R at a MOI of 10. Cells were collected at 6 h post infection. IFNA (FIG. 58A), CCL4 (FIG. 58B), and CCL5 (FIG. 58C) gene expressions were determined by quantitative RT-PCR.

FIGS. 59A-59C show that MVAΔE5R infection of BMDCs induce high levels of IFNB and viral E3R gene expression and IFN-β protein secretion from BMDCs. FIGS. 59A and 59B show real-time quantitative PCR (RT-PCR) analyses of IFNB (FIG. 59A) and viral E3R (FIG. 59B) gene expression induced by MVAΔE5R or Heat-inactivated MVAΔE5R (“Heat-iMVAΔE5R”). BMDCs were generated by culturing bone marrow cells in the presence of mGM-CSF. Cells were infected with either MVAΔE5R or Heat-iMVAΔE5R virus at MOIs of 0.25, 1, 3, or 10. Cells were washed after 1 h infection and fresh medium was added. Cells were collected at 14 h post infection. IFNB and E3 gene expressions were determined by RT-PCR. FIG. 59C BMDCs were infected with either MVAΔE5R or Heat-iMVAΔE5R virus at MOIs of 0.25, 1, 3, or 10. Supernatants were collected at 14 h post infection. The concentrations of IFN-β in the supernatants were measured by ELISA.

FIGS. 60A-60D show that MVAΔE5R-induced IFNB gene expression and IFN-β secretion was dependent on cGAS. FIG. 60A shows IFNA induction by MVAΔE5R in WT and cGAS^{-/-} BMDCs. FIG. 60B shows IFNA induction by MVAΔE5R in WT and cGAS^{-/-} BMDCs. FIG. 60C shows vaccinia E3R gene expression in WT and cGAS^{-/-} BMDCs infected with MVAΔE5R. BMDCs were infected with either MVA or MVAΔE5R at a MOI of 10. Cells were collected at 6 h post infection. RNAs were extracted. Real-time quantitative PCR analysis was performed. FIG. 60D shows MVAΔE5R induces higher levels of IFN-β protein secretion compared with Heat-iMVA or Heat-iMVAΔE5R, and the induction is completely dependent on cGAS. WT or cGAS^{-/-} BMDCs were infected with either MVA, MVAΔE5R at a MOI of 10, Heat-iMVA, or Heat-iMVAΔE5R at an equivalent of MOI. Supernatants were collected at 8 and 16 h post infection. IFN-β protein levels in the supernatants were measured by ELISA.

FIGS. 61A and 61B show MVAΔE5R-induced IFNB gene expression and protein secretion from BMDCs is dependent on STING. FIG. 61A show BMDCs from WT or STING^{Gt/Gt} mice were infected with either MVA, MVAΔE5R, or Heat-iMVAΔE5R. Cells were collected at 8 h post infection and RT-PCR analysis was performed. Fold induction of IFNB gene expression is shown. FIG. 61B shows bone marrow derived macrophages (BMDMs) were generated by culturing bone marrow cells from WT and STING^{Gt/Gt} mice in the presence of M-CSF (macrophage colony stimulating factor). BMDMs were infected with either MVA, MVAΔE5R, Heat-iMVA, or Heat-iMVAΔE5R. Supernatants were collected at 16 h post infection and IFN-β protein levels were measured by ELISA.

FIGS. 62A-62D show that MVAΔE5R-induced IFN-β protein secretion requires IRF3, IRF7 and IFNAR. FIG. 62A shows that WT or IRF3^{-/-} BMDCs were infected with either MVA, MVAΔE5R, Heat-iMVA, or Heat-iMVAΔE5R. Supernatants were collected at 8 and 16 h post infection. IFN-β levels were determined by ELISA. FIG. 62B shows that WT or IRF3^{-/-} BMDMs were infected with either MVA, MVAΔE5R, Heat-iMVA, or Heat-iMVAΔE5R. Supernatants were collected at 8 and 16 h post infection. IFN-β levels were determined by ELISA. FIG. 62C shows that WT or IRF7^{-/-} BMDCs were infected with MVAΔE5R at a MOI of 10. Supernatants were collected at 21 h post infection. IFN-β levels in the supernatants were determined by ELISA. FIG. 62D shows that WT, cGAS^{-/-}, or IFNAR^{-/-} BMDCs were infected with either MVA, MVAΔE5R, Heat-iMVA, or Heat-IMVAΔE5R. Supernatants were collected at 16 h post infection. IFN-β levels were determined by ELISA.

FIGS. 63A-63C demonstrate that WT VACV-induced cGAS degradation is mediated through a proteasome-dependent pathway. FIG. 63A shows that murine embryonic fibroblasts were pretreated with either cycloheximide (CHX), a proteasomal inhibitor, MG132, a pan-caspase inhibitor, Z-VAD, or an AKT1/2 inhibitor VIII for 30 min. MEFs were then infected with WT VACV in the presence of each drug. Cells were collected at 6 h post infection. Western blot analysis was performed with anti-cGAS and anti-GAPDH antibodies. FIG. 63B shows that MEFs were treated with DMSO or MG132. Cells were collected at 2, 4, and 6 h post treatment. Western blot analysis was performed with anti-cGAS and anti-GAPDH antibodies. FIG. 63C demonstrates that WT VACV infection of BMDCs resulted in cGAS degradation, whereas VACVΔE5R did not. In the presence of MG132, WT VACV-induced cGAS degradation was blocked. BMDCs were infected with WT VACV or VACV at MOI of 10 in the presence or absence of MG132. Cells were collected at 2, 4, and 6 h post infection. Western blot analyses were performed using anti-cGAS and anti-GAPDH antibodies.

FIG. 64 demonstrates that the E5R gene in MVA is important in mediating cGAS degradation in BMDCs. BMDCs were infected with either MVA or MVAΔE5R at a MOI of 10. Cells were collected at 2, 4, 6, 8, and 12 h post infection. Western blot analysis was performed using anti-cGAS and anti-GAPDH antibodies.

FIG. 65 demonstrates that MVAΔE5R induces higher levels of phosphorylated Stat2 compared with MVA. BMDCs were infected with either MVA or MVAΔE5R at a MOI of 10. Cells were collected at 2, 4, 6, 8, and 12 h post infection. Western blot analysis was performed using anti-phospho-STAT2, anti-STAT2, and anti-GAPDH antibodies.

FIG. 66 shows that MVAΔE5R induces high levels of cGAMP production in infected BMDCs. 2.5×10⁶ BMDCs were infected with either MVA or MVAΔE5R at a MOI of 10. Cells were collected at 2, 4, 6 and 8 h post infection. cGAMP concentrations were measured by incubating cell lysates with permeabilized differentiated THP1-Dual™ cells, which were derived from the human THP-1 monocyte cell line by stable integration of two inducible reporter constructs. Supernatants were collected at 24 h, and luciferase activities (as an indication for IRF pathway activation) were measured. cGAMP levels were calculated by comparing with cGAMP standards.

FIGS. 67A and 67B show that MVAΔE5R induces IFN-β protein secretion from plasmacytoid dendritic cells. FIG. 67A shows 1.2×10⁵ pDCs (B220⁺PDCA-1⁺) sorted from splenocytes were infected with either MVA, Heat-iMVA, or MVAΔE5R. Non-infected splenocytes were included as a control. Supernatants were collected at 18 h post infection. IFN-β levels in the supernatants were measured by ELISA. FIG. 67B shows 4×10⁵ pDCs (B220⁺PDCA-1⁺) sorted from Flt3L-BMDCs were infected with either MVA, Heat-iMVA, or MVAΔE5R. Non-infected sorted pDCs were included as a control. Supernatants were collected at 18 h post infection. IFN-β levels in the supernatants were measured by ELISA.

FIG. 68 shows that MVAΔE5R-induced IFN-β secretion from pDCs is dependent on cGAS. pDCs were sorted from Flt3L-cultured BMDCs (B220⁺PDCA-1⁺) obtained from WT, cGAS^{-/-}, or MyD88^{-/-} mice. 2×10⁵ cells were infected with either MVA or MVAΔE5R. NT control was included. Supernatants were collected at 18 h post infection. IFN-β levels in the supernatants were measured by ELISA.

FIG. 69 shows that MVAΔE5R infection induces IFN-β protein secretion from CD103⁺ DCs through a cGAS-dependent pathway. CD103⁺ DCs were sorted from Flt3L-cultured BMDCs (CD11c⁺CD103⁺) obtained from WT, cGAS^{-/-}, or MyD88^{-/-} mice. 2×10⁵ cells were infected with either MVA or MVAΔE5R. NT control was included. Supernatants were collected at 18 h post infection. IFN-β levels in the supernatants were measured by ELISA.

FIG. 70 shows that MVAΔE5R infection of BMDCs results in lower levels of cell death compared with MVA. BMDCs were infected with either MVA or MVAΔE5R at a MOI of 10. Cells were harvested at 16 h post infection and stained with LIVE/DEAD fixable viability dye and subjected for flow cytometry analysis.

FIGS. 71A and 71B show that MVAΔE5R infection promotes DC maturation in a cGAS-dependent manner. BMDCs from WT and cGAS^{-/-} mice were infected with MVA-OVA or MVAΔE5R-OVA at MOI of 10. Cells were collected at 16 h post infection and stained for DC maturation markers: CD40 (FIG. 71A) and CD86 (FIG. 71B).

FIGS. 72A-72G shows that MVAΔE5R infection of BMDCs promotes antigen cross-presentation as measured by T cell activation. BMDCs were infected with either MVA, MVAΔE5R, Heat-iMVA, or Heat-iMVAΔE5R at MOI of 3 for 3 h and then incubated with OVA for 3 h. OVA was washed away and cells were then incubated with OT-I cells (which recognizes OVA₂₅₇₋₂₆₄ SIINFEKL peptide) for 3 days. OT-1 cells were stained with anti-CD69 and anti-CD8 antibodies and analyzed by flow cytometry. Supernatants were collected and IFN-γ levels were determined by ELISA. Dot plots demonstrate CD8+ cells expressing CD69.

FIG. 73 shows that MVAΔE5R infection of BMDCs promotes antigen cross-presentation as measured by IFN-γ production by activated T cells. BMDCs were infected with either MVA, MVAΔE5R, Heat-iMVA, or Heat-iMVAΔE5R at MOI of 3 for 3 h and then incubated with OVA for 3 h. OVA was washed away and cells were then incubated with OT-I cells (which recognizes OVA₂₅₇₋₂₆₄ SIINFEKL peptide) for 3 days. OT-1 cells were stained with anti-CD69 and anti-CD8 antibodies and analyzed by flow cytometry. Supernatants were collected and IFN-γ levels were determined by ELISA. FIG. 73 shows IFN-γ levels in the supernatants of the BMDC:T cells co-culture.

FIG. 74 shows that VACVΔE5R infection of BMDCs promotes antigen cross-presentation as measured by IFN-γ production by activated T cells. BMDCs were infected with either MVA, MVAΔE5R, or VACVΔE5R at MOI of 3 for 3 h; or BMDCs were incubated with cGAMP or mock control for 3h. BMDCs were subsequently incubated with OVA for 3 h and then the OVA was washed away. Cells were then incubated with OT-I cells (which recognizes OVA₂₅₇₋₂₆₄ SIINFEKL peptide) for 3 days. Supernatants were collected and IFN-γ levels were determined by ELISA.

FIGS. 75A-75C show that deletion of the E5R gene from MVA improves vaccination efficacy. FIG. 75A is a scheme of vaccination strategy. On day 0, C57BL/6J mice were vaccinated with MVA-OVA or MVAΔE5R-OVA at 2×10⁷ pfu either through skin scarification or intradermal injection. Spleens were harvested from euthanized mice one week later and co-cultured with OVA₂₅₇₋₂₆₄ (SIINFEKL) peptide (10 g/ml) pulsed BMDCs for 12 h. The intracellular IFN-γ levels in CD8⁺ T cells was then measured by flow cytometry. (* p<0.05; ** p<0.01). FIG. 75B shows activated CD8⁺ T cells after vaccination through skin scarification with MVA-OVA or MVAΔE5R-OVA. FIG. 75C shows activated CD8⁺ T cells after vaccination through intradermal injection of MVA-OVA or MVAΔE5R-OVA.

FIGS. 76A and 76B show that MVAΔE5R infection induces IFNB gene expression and IFN-β secretion from murine primary fibroblasts in a cGAS-dependent manner. Skin dermal fibroblasts were generated from female WT and cGAS^{-/-} C57BL/6J mice. Cells were infected with either MVA, MVAΔE5R, Heat-iMVA, or Heat-iMVAΔE5R. Cells and supernatants were collected at 16 h post infection. FIG. 76A shows RT-PCR results of IFNB gene expression in WT and cGAS^{-/-} cells. FIG. 76B shows IFN-β protein levels in the supernatants of infected WT and cGAS^{-/-} cells as measured by ELISA.

FIGS. 77A-77D show that MVAΔE5R gains its capacity to replicate its DNA in cGAS- or IFNAR1-deficient skin primary dermal fibroblasts. Skin primary dermal fibroblasts from WT, cGAS^{-/-} or IFNAR1^{-/-} mice were infected with either MVA or MVAΔE5R at a MOI of 3. Cells were collected 1, 4, 10 and 24 h post infection. Viral DNA copy numbers were determined by quantitative PCR. FIG. 77A shows DNA copy numbers in MVA-infected WT, cGAS^{-/-} or IFNAR1^{-/-} skin dermal fibroblasts. FIG. 77B shows the fold-change compared with the DNA copy numbers at 1 h post infection with MVA. FIG. 77C shows DNA copy numbers in MVAΔE5R-infected WT, cGAS^{-/-} or IFNAR1^{-/-} skin dermal fibroblasts. FIG. 77D shows the fold-change compared with the DNA copy numbers at 1 h post infection with MVAΔE5R.

FIGS. 78A-78D show that MVAΔE5R gains its capacity to generate infectious progeny viruses in cGAS-deficient skin primary dermal fibroblasts. Skin primary dermal fibroblasts from WT, cGAS^{-/-} or IFNAR1^{-/-} mice were infected with either MVA or MVAΔE5R at a MOI of 0.05. Cells were collected 1, 24, and 48 h post infection. Viral titers were determined by titrating on BHK21 cells. FIG. 78A shows MVA titers over time after infection in WT, cGAS^{-/-} or IFNAR1^{-/-} skin dermal fibroblasts. FIG. 78 shows the fold-change compared with the viral titers at 1 h post infection with MVA. FIG. 78C shows MVAΔE5R titers over time after infection in WT, cGAS^{-/-} or IFNAR1^{-/-} skin dermal fibroblasts. FIG. 78D shows the fold-change compared with the viral titers at 1 h post infection with MVAΔE5R.

FIGS. 79A and 79B show that MVAΔE5R infection of murine melanoma cells induce IFNB gene expression and IFN-β protein secretion in a STING-dependent manner. FIG. 79A shows RT-PCR results of IFNB induction by MVAΔE5R in WT B16-F10 murine melanoma cells and STING^{-/-} B16-F10 cells. WT and STING^{-/-} B16-F10 cells were infected with either MVA or MVAΔE5R at a MOI of 10. Cells were collected at 18 h post infection. RNAs were extracted and quantitative real-time PCR analysis was performed. FIG. 79B shows ELISA results of IFN-β protein levels in the supernatants of WT and STING^{-/-} B16-F10 cells infected with either MVA or MVAΔE5R collected at 18 h post infection.

FIG. 80 shows that MVAΔE5R infection of murine melanoma cells induces ATP release, which is a hallmark of immunogenic cell death. B16-F10 cells were infected with WT vaccinia, MVA, or MVAΔE5R at a MOI of 10. Supernatants were collected at 48 h post infection. ATP levels were determined by using ATPlite 1step Luminescence ATP Detection Assay System (PerkinElmer, Waltham, MA).

FIG. 81 shows a scheme of generating recombinant MVAΔE5R expressing hFlt3L and hOX40L through homologous recombination at the E4L and E6R loci of the MVA genome. pUC57 vector is used to insert a single expression cassette designed to express both hFlt3L and hOX40L using the vaccinia viral synthetic early and late promoter (PsE/L). The coding sequence of the hFlt3L and hOX40L was separated by a cassette including a furin cleavage site followed by a Pep2A sequence. Homologous recombination that occurred at the E4L and E6R loci results in the insertion of expression cassette for hFlt3L and hOX40L.

FIGS. 82A and 82B show that the recombinant MVAΔE5R-hFlt3L-hOX40L virus has the expected insertion as determined by PCR analysis. Lane 1 shows the Fermentas 1 kb plus DNA ladder. Lane 2 shows a band with expected size of 1120 bp using F0/R5 primer pairs. Lane 3 shows a band with expected size of 1166 bp using F2/R2 primer pairs. Lane 4 shows a band with expected size of 1136 bp using F5/RO primer pairs.

FIGS. 83A-83C show that MVAΔE5R-hFlt3L-hOX40L virus expresses both hFlt3L and hOX40L on the surface of infected cells. FIG. 83A are dot plots of FACS analysis of hFlt3L expression on the Y axis and hOX40L expression on the X axis of BHK21 cells infected with either MVA or MVAΔE5R-hFlt3L-hOX40L for 24 h. Cells were infected at a MOI of 10. A mock infection, no virus control was included. FIG. 83B are dot plots of FACS analysis of hFlt3L expression on the Y axis and hOX40L expression on the X axis of murine B16-F10 melanoma infected with either MVA or MVAΔE5R-hFlt3L-hOX40L for 24 h. Cells were infected at a MOI of 10. A mock infection, no virus control was included. FIG. 83C are dot plots of FACS analysis of hFlt3L expression on the Y axis and hOX40L expression on the X axis of human melanoma cells SK-MEL28 infected with either MVA or MVAΔE5R-hFlt3L-hOX40L for 24 h. Cells were infected at a MOI of 10. A mock infection, no virus control was included.

FIG. 84 shows Western blot results of the expression of hFlt3L and hOX40L in MVAΔE5R-hFlt3L-hOX40L-infected BHK21 cells. BHK21 cells were either mock infected, or infected with MVA or MVAΔE5R-hFlt3L-hOX40L. Cells lysates were collected at 24 h post infection. Western blot analysis was performed using anti-hFlt3L and anti-hOX40L antibodies.

FIGS. 85A and 85B show a scheme to generate VACV-TK⁻-anti-CTLA-4-ΔE5R-hFlt3L-mOX40L. FIG. 85A shows a schematic diagram of pCB vector with a single expression cassette designed to express the heavy chain and light of the antibody using the vaccinia viral synthetic early and late promoter (PsE/L). The coding sequence of the heavy chain (muIgG2a) and the light chain of 9D9 was separated by a cassette including a furin cleavage site followed by a 2A peptide (Pep2A) sequence to enables ribosome skipping. The pCB plasmid containing the anti-mu-CTLA-4 gene under the control of the vaccinia PsE/L as well as the E. coli xanthine-guanine phosphoribosyl transferase gene (gpt) under the control of vaccinia P7.5 promoter flanked by the thymidine kinase (TK) gene on either side. Recombinant virus expressing anti-mu-CTLA-4 from TK locus was generated through homologous recombination at the TK locus between pCB plasmid DNA and viral genomic DNA. FIG. 85B is the schematic diagram of using the vaccinia viral synthetic early and late promoter (PsE/L) to express both human Flt3L and murine OX40L as a fusion protein in a single expression cassette. The coding sequence of human Flt3L and murine OX40L was separated by a cassette including a furin cleavage site followed by a 2A peptide (Pep2A) sequence. A pUC57 plasmid containing human Flt3L and murine OX40L fusion gene flanked by the E4L and E6R genes on either side was constructed. Recombinant virus expressing human Flt3L and murine OX40L fusion protein from E5R locus was generated through homologous recombination at E4L and E6R loci between pUC57 plasmid and viral genomic DNA.

FIGS. 86A-86C show the scheme and the results of PCR analysis to verify the recombinant vaccinia virus VACV-TK⁻-anti-muCTLA-4-E5R⁻-hFlt3L-mOX40L, as well as the Western blot results on the expression of anti-CTLA-4 antibodies by the cells infected with the recombinant virus. FIG. 86A shows the schematic diagram of the primers used to amplify the different gene fragments from the inserted expression cassette of pUC57 expression plasmid. Primer pair f1/r1 was used to generate a 2795 bp PCR fragment, which contains the whole expression cassette. This primer pair was also used to check the purity of the recombinant virus. Primer pair f0/r5 was used to confirm the expression cassette was inserted into the right position in virus genome. Human Flt3L gene was amplified using primer pair f1/r3 or fo/r2 from VACV-TK⁻-anti-muCTLA-4-E5R⁻-hFlt3L-mOX40L. Murine OX40L gene was generated using primer pair f4/r1. And finally, pCB-R4 and TK-F5 primer pair was used to generate the anti-mu-CTLA-4 gene inserted in TK locus from VACV-TK⁻-anti-muCTLA-4-E5R⁻-hFlt3L-mOX40L or MVA-TK⁻-anti-muCTLA-4-E5R⁻-hFlt3L-mOX40L recombinant virus. FIG. 86B shows the gel image of the PCR results using the primer pairs described in FIG. 86A and a table that displays the predicated sizes of the amplified DNA fragments with the primer pairs. FIG. 86C shows Western blot results of human SK-MEL-28 melanoma cells mock infected or infected with E3LΔ83N-TK⁻-vector, E3LΔ83N-TK⁻-hFlt3L-anti-muCTLA-4, E3LΔ83N-TK⁻-hFlt3L-anti-muCTLA-4-C7L⁻-mOX40L, VACV, VACV-TK⁻-anti-muCTLA-4-C7L⁻-mOX40L, or VACV-TK⁻-anti-muCTLA-4-E5R⁻-hFlt3L-mOX40L at a MOI of 10. Cell lysates were collected at 24 hours post-infection, and polypeptides were separated using 10% SDS-PAGE. HRP-linked anti-mouse IgG (heavy and light chain) antibody was used to detect full-length (FL), heavy chain (HC), and light chain (LC) of anti-muCTLA-4 antibodies.

FIG. 87 shows the protein sequence alignments of E5 orthologs from multiple members of the poxvirus family.

FIG. 88A shows the protein sequence alignments of E5 from vaccinia virus and Modified vaccinia virus Ankara (MVA). FIG. 88B shows the protein sequence alignments of E5 from vaccinia virus and myxoma virus.

FIGS. 89A and 89B show that myxoma virus M31R inhibits cGAS and STING induced IFN-β pathway. FIG. 89A shows that HEK293T cells were transfected with plasmids expressing murine cGAS, human STING together with either E5R, M31R or pcDNA vector control expressing plasmid. After 24 h, Luciferase signals were determined.

FIG. 89B shows that HEK293T cells were transfected with plasmids expressing murine STING together with either E5R, M31R or pcDNA vector control expressing plasmid. After 24 h, Luciferase signal were determined.

FIGS. 90A and 90B show that vaccinia E5 promotes cGAS ubiquitination. FIG. 90A shows that HEK293T cells were transfected with Flag-cGAS and HA-ubiquitin. After 24 h, cells were infected with either WT VACV or VACVΔE5R. Cell lysis were collected after 6 h. cGAS was immunoprecipitated with anti-Flag antibody and ubiquitination was detected by anti-HA antibody. FIG. 90B shows Western blot analysis of cGAS and 3-actin in whole cell lysates (WCL).

FIGS. 91A-91C are graphical representations of data showing IT MVAΔE5R delays tumor growth and prolongs survival in murine B16-F10 melanoma unilateral tumor implantation model. FIG. 91A is a scheme of tumor implantation and treatment for a B16-F10 unilateral tumor implantation model. Briefly, 5×10⁵ B16-F10 melanoma cells were implanted intradermally to the right flank of C57B/6J mice. Eight days post tumor implantation, PBS, 4×10⁷ pfu of MVA, MVAΔE5R or Heat-iMVA were intratumorally injected twice weekly. The tumor sizes were measured and the survival of mice was monitored. FIG. 91B is a graph of the Kaplan-Meier survival curve of tumor-bearing mice treated with either PBS, MVA, MVAΔE5R, or Heat-iMVA, treatments. (n=5, *P<0.05; **P<0.01; Mantel-Cox test). FIG. 91C is a table showing median survival of mice treated with either PBS, MVA, MVAΔE5R, or Heat-iMVA.

FIGS. 92A-92E show that MVAΔC7L-hFlt3L-TK(−)mOX40LΔE5R infection of BMDCs results in higher levels of IFNB gene expression and IFN-□ protein secretion compared with MVAΔE5R. FIGS. 92A-92C show schematic diagrams of generating MVAΔC7L-hFlt3L-TK(−)-mOX40LΔE5R virus. The first step involves the generation of MVAΔC7L-hFlt3L through homologous recombination at the C8L and C6R loci, replacing C7L gene with hFlt3L under the control of PsE/L promoter (FIG. 92A). The second step involves the generation of MVAΔC7L-hFlt3L-TK(−)-mOX40L through homologous recombination at the TK loci, replacing TK gene with mOX40L under the control of PsE/L promoter (FIG. 92B). The resulting virus was described in FIGS. 5A and 5B. The third step is to generate MVAΔC7L-hFlt3L-TK(−)-mOX40LΔE5R through homologous recombination at the E4L and E6R loci, replacing E5R gene with mCherry under the control of P7.5 promoter (FIG. 92C). FIG. 92D shows RT-PCR results of IFNB gene expression in BMDCs infected with either MVAΔE5R, MVAΔC7L-hFlt3L-TK(−)-mOX40L, or MVAΔC7L-hFlt3L-TK(−)mOX40LΔE5R. WT and IFNAR^{-/-} BMDCs were mock-infected or infected with MVAΔE5R, MVAΔC7L-hFlt3L-TK(−)-mOX40L, or MVAΔC7L-hFlt3L-TK(−)mOX40LΔE5R at a MOI of 10. Cells were collected at 16 h post infection and RT-PCR was performed. FIG. 92E shows ELISA results of IFN-□ protein levels in the supernatants of BMDCs infected with either MVAΔE5R, MVAΔC7L-hFlt3L-TK(−)-mOX40L, or MVAΔC7L-hFlt3L-TK(−)mOX40LΔE5R. WT and IFNAR^{-/-} BMDCs were mock-infected or infected with MVAΔE5R, MVAΔC7L-hFlt3L-TK(−)-mOX40L, or MVAΔC7L-hFlt3L-TK(−)mOX40LΔE5R at a MOI of 10. Supernatants were collected at 16 h post infection and ELISA was performed to measure IFN-□ protein levels.

FIGS. 93A and 93B show the scheme of generating MVAΔC7LΔE5R-hFlt3L-mOX40L and MVAΔC7L-OVA-ΔE5R-hFlt3L-mOX40L. FIG. 93A shows the scheme of generating MVAΔC7LΔE5R-hFlt3L-mOX40L. pUC57 plasmid is constructed to use the vaccinia viral synthetic early and late promoter (PsE/L) to express both human Flt3L and murine OX40L as a fusion protein in a single expression cassette. The coding sequence of human Flt3L and murine OX40L was separated by a cassette including a furin cleavage site followed by a 2A peptide (Pep2A) sequence. Recombinant virus expressing human Flt3L and murine OX40L fusion protein from E5R locus was generated through homologous recombination at E4L and E5R loci between pUC57 plasmid and MVAΔC7L viral genome. FIG. 93B shows the scheme of generating MVAΔC7L-OVA-ΔE5R-hFlt3L-mOX40L. Recombinant virus expressing human Flt3L and murine OX40L fusion protein from E5R locus was generated through homologous recombination at E4L and E5R loci between pUC57 plasmid and MVAΔC7L-OVA viral genome.

FIGS. 94A and 94B. FIG. 94A: Dual-luciferase assay of HEK293T cells transfected with ISRE-firefly luciferase reporter, a control plasmid pRL-TK that expresses Renilla luciferase, together with either myxoma M62R, Myxoma M62R-HA, Myxoma M64R, Myxoma M64R-HA, vaccinia C7L-expressing or control plasmid. 24 h post transfection, cells were treated with IFN-β for another 24 h before harvesting. FIG. 94B: Dual-luciferase assay of HEK293T cells transfected with IFNB-firefly luciferase reporter, a control plasmid pRL-TK that expresses Renilla luciferase, and STING-expressing plasmid, together with either myxoma M62R, Myxoma M62R-HA, Myxoma M64R, Myxoma M64R-HA, vaccinia C7L-expressing, or control plasmid. Cells were harvested at 24 h post transfection.

FIG. 95 shows a scheme of generating recombinant MVAΔE5R expressing hFlt3L and mOX40L through homologous recombination at the E4L and E6R loci of the MVA genome. pUC57 vector was used to insert a single expression cassette designed to express both hFlt3L and mOX40L using the vaccinia viral synthetic early and late promoter (PsE/L). The coding sequence of the hFlt3L and mOX40L was separated by a cassette including a furin cleavage site followed by a Pep2A sequence. Homologous recombination that occurred at the E4L and E6R loci results in the insertion of expression cassette for hFlt3L and mOX40L.

FIGS. 96A and 96B show that MVAΔE5R-hFlt3L-mOX40L virus expresses both hFlt3L and mOX40L on the surface of infected cells. FIG. 96A shows the dot plots of FACS analysis of hFlt3L expression on the Y axis and mOX40L expression on the X axis of BHK21 cells, murine melanoma cells B16-F10, or human melanoma cells SK-MEL28. Cells were infected with either MVA or MVAΔE5R-hFlt3L-mOX40L at a MOI of 10 for 24 h. No virus mock infection control was included. FIG. 96B shows the graphs of medium fluorescence intensity (MFI) of human Flt3L and murine OX40L on infected BHK21, B16-F10, and SK-MEL28 cells infected with either MVA, MVAΔE5R-hFlt3L-mOX40L, or PBS.

FIGS. 97-102 are a series of graphical representations of data showing that intratumoral injection of MVAΔE5R-hFlt3L-mOX40L generated more activated tumor-infiltrating effector T cells in injected and non-injected distant tumors as well as in the spleens compared with MVA, MVAΔE5R, or Heat-iMVA in a B16-F10 bilateral tumor model. FIG. 97 shows the experimental scheme. Briefly, B16-F10 melanoma cells were implanted intradermally to the left and right flanks of C57B/6J mice (5×10⁵ to the right flank and 2.5×10⁵ to the left flank). Seven days post tumor implantation, 2×10⁷ pfu of either MVAΔE5R-hFlt3L-mOX40L, MVA, MVAΔE5R, an equivalent amount of Heat-iMVA, or PBS was intratumorally (IT) injected into the larger tumors on the right flank twice, three days apart. Spleens were harvested at 2 days post second injection, ELISPOT analyses were performed to evaluate tumor-specific T cells in the spleens. Both injected and non-injected tumors were also isolated and tumor infiltrating lymphocytes were analyzed by FACS.

FIGS. 98A-98B. ELISPOT assay was performed by co-culturing irradiated B16-F10 cells (150,000) and splenocytes (1,000,000) in a 96-well plate. FIG. 98A shows the image of ELISPOT of triplicate samples from left to right. FIG. 98B shows the graph of IFN-γ⁺ spots per 1,000,000 purified CD8⁺ T cells. Each bar represents spleen sample from individual mouse (n=3-8) (*P<0.05; **P<0.01, t test).

FIGS. 99A-99C. FIG. 99A shows the representative dot plots of Granzyme B⁺ CD8⁺ T cells in non-injected tumors after treatment with either MVAΔE5R-hFlt3L-mOX40L, Heat-iMVA, or PBS. FIG. 99B shows the graph of percentages of CD8⁺ T cells out of CD3⁺ cells. Data are means±SEM (n=5-9). (**P<0.01; ***P<0.001, t test). FIG. 99C shows the graph of percentages of Granzyme B⁺CD8⁺ T cells out of CD8⁺ cells). Data are means±SEM (n=5-9) (**P<0.01; ***P<0.001, t test).

FIGS. 100A-100C. FIG. 100A shows the representative dot plots of Granzyme B⁺ CD4⁺ T cells in non-injected tumors after treatment with either MVAΔE5R-hFlt3L-mOX40L, Heat-iMVA, or PBS. FIG. 100B shows the graph of percentages of CD4⁺ T cells out of CD3⁺ cells. Data are means±SEM (n=5-9). (*P<0.05; t test). FIG. 100C shows the graph of percentages of Granzyme B⁺ CD4⁺ T cells out of CD4⁺ cells). Data are means±SEM (n=5-9) (**P<0.01; ***P<0.001, t test).

FIGS. 101A-101C. FIG. 101A shows the representative dot plots of Granzyme B⁺ CD8⁺ T cells in the injected tumors after treatment with either MVAΔE5R-hFlt3L-mOX40L, Heat-iMVA, or PBS. FIG. 101B shows the graph of percentages of CD8⁺ T cells out of CD3⁺ cells. Data are means±SEM (n=5-9). ****P<0.0001, t test). FIG. 101C shows the graph of percentages of Granzyme B⁺ CD8⁺ T cells out of CD8⁺ cells). Data are means±SEM (n=5-9) (*P<0.05; ****P<0.0001, t test).

FIGS. 102A-102C. FIG. 102A shows the representative dot plots of Granzyme B⁺ CD4⁺ T cells in the injected tumors after treatment with either MVAΔE5R-hFlt3L-mOX40L, Heat-iMVA, or PBS. FIG. 102B shows the graph of percentages of CD4⁺ T cells out of CD3⁺ cells. Data are means±SEM (n=5-9). (**P<0.01; t test). FIG. 102C shows the graph of percentages of Granzyme B⁺ CD4⁺ T cells out of CD4⁺ cells). Data are means±SEM (n=5-9) (*P<0.05; **P<0.01; ****P<0.0001, t test).

FIGS. 103A-104C are a series of graphical representations of data showing that intratumoral injection of MVAΔE5R-hFlt3L-mOX40L reduced FoxP3⁺CD4⁺ regulatory T cells in the injected tumors, but not in the non-injected tumors. FIG. 103A shows the representative dot plots of FoxP3⁺CD4⁺ cells in the injected tumors after treatment with either MVAΔE5R-hFlt3L-mOX40L, Heat-iMVA, or PBS. FIG. 103B shows the graph of percentages of FoxP3⁺CD4⁺ T cells out of CD4⁺ cells. Data are means±SEM (n=5-9). (**P<0.01; ***P<0.001, t test). FIG. 103C shows the graph of absolute numbers of FoxP3⁺CD4⁺ T cells per gram of tumor. Data are means±SEM (n=5-9). (*P<0.05, t test).

FIG. 104A shows the representative dot plots of FoxP3⁺CD4⁺ cells in the non-injected tumors after treatment with either MVAΔE5R-hFlt3L-mOX40L, Heat-iMVA, or PBS. FIG. 104B shows the graph of percentages of FoxP3⁺CD4⁺ T cells out of CD4⁺ cells. Data are means±SEM (n=5-9). FIG. 104C shows the graph of absolute numbers of FoxP3⁺CD4⁺ T cells per gram of tumor. Data are means±SEM (n=5-9).

FIGS. 105A to 109C are a series of graphical representations of data showing that intratumoral injection of MVAΔE5R-hFlt3L-mOX40L preferentially reduces OX40⁺FoxP3⁺CD4⁺ regulatory T cells in the injected tumors. FIG. 105A shows the representative dot plots of OX40⁺FoxP3⁺CD4⁺ cells in the non-injected tumors after treatment with either MVAΔE5R-hFlt3L-mOX40L, Heat-iMVA, or PBS. FIG. 105B shows the graph of percentages of OX40⁺FoxP3⁺CD4⁺ T cells out of CD4⁺ cells in the injected tumors. Data are means±SEM (n=5-9). (**P<0.01; ***P<0.001, t test). FIG. 105C shows the graph of absolute numbers of OX40⁺FoxP3⁺CD4⁺ T cells per gram of tumor. Data are means±SEM (n=5-9). (*P<0.05, t test).

FIG. 106A shows the representative dot plots of OX40⁺FoxP3⁺CD4⁺ cells in the non-injected tumors after treatment with either MVAΔE5R-hFlt3L-mOX40L, Heat-iMVA, or PBS. FIG. 106B shows the graph of percentages of OX40⁺FoxP3⁺CD4⁺ T cells out of CD4⁺ cells. Data are means±SEM (n=5-9). FIG. 106C shows the graph of absolute numbers of OX40⁺FoxP3⁺CD4⁺ T cells per gram of tumor. Data are means±SEM (n=5-9).

FIG. 107A shows the representative dot plots of OX40⁺FoxP3⁻CD4⁺ cells in the non-injected tumors after treatment with either MVAΔE5R-hFlt3L-mOX40L, Heat-iMVA, or PBS. FIG. 107B shows the graph of percentages of OX40⁺FoxP3⁻ CD4⁺ T cells out of CD4⁺ cells. Data are means±SEM (n=5-9). FIG. 107C shows the graph of absolute numbers of OX40⁺FoxP3⁻CD4⁺ T cells per gram of tumor. Data are means±SEM (n=5-9).

FIG. 108 shows the representative dot plots of OX40⁺CD8⁺ cells in the non-injected tumors after treatment with either MVAΔE5R-hFlt3L-mOX40L, Heat-iMVA, or PBS.

FIGS. 109A-109C are a series of graphical representations of data showing that intratumoral injection of MVAΔE5R-hFlt3L-mOX40L results in more reduction of FoxP3⁺CD4⁺ regulatory T cells in the injected tumors compared with MVAΔE5R. FIG. 109A shows the representative dot plots of FoxP3⁺CD4⁺ cells in the injected tumors after treatment with either MVAΔE5R-hFlt3L-mOX40L, MVAΔE5R, or PBS. FIG. 109B shows the graph of percentages of FoxP3⁺CD4⁺ T cells out of CD4⁺ cells in the injected tumors. Data are means±SEM (n=5-9). (*P<0.05; **P<0.01, t test). FIG. 109C shows the graph of absolute numbers of FoxP3⁺CD4⁺ T cells per gram of tumor. Data are means±SEM (n=5-9). (**P<0.01, t test).

FIGS. 110-115C are a series of graphical representations of data showing that intratumoral injection of MVAΔE5R-hFlt3L-mOX40L generated more activated tumor-infiltrating effector CD8⁺ and CD4⁺ T cells in the injected and non-injected distant tumors in OX40^{-/-} mice compared with WT mice in a B16-F10 bilateral murine melanoma model. FIG. 110 shows the experimental scheme. Briefly, B16-F10 melanoma cells were implanted intradermally to the left and right flanks of C57B/6J mice (5×10⁵ to the right flank and 2.5×10⁵ to the left flank). Ten days post tumor implantation, intratumoral injections (4×10⁷ pfu) of either MVAΔE5R-hFlt3L-mOX40L or PBS were performed to the larger tumors on the right flank twice, three days apart. Both the injected and non-injected distant tumors were harvested at 2 days post second injection and tumor-infiltrating lymphocytes were analyzed by FACS.

FIG. 111A shows the representative dot plots of Granzyme B⁺ CD8⁺ T cells in the injected tumors from WT and OX40^{-/-} mice after treatment with either MVAΔE5R-hFlt3L-mOX40L or PBS. FIG. 111B shows the graph of percentages of CD8⁺ T cells out of CD3⁺ cells. Data are means±SEM (n=4-10). FIG. 111C shows the graph of absolute numbers of CD8⁺ T cells per gram of tumor. Data are means±SEM (n=4-10). FIG. 111D shows the graph of percentages of Granzyme B⁺ CD8⁺ T cells out of CD8⁺ cells. Data are means±SEM (n=4-10). FIG. 111E shows the graph of absolute numbers of Granzyme B⁺ CD8⁺ T cells per gram of tumor. Data are means±SEM (n=4-10).

FIG. 112A shows the representative dot plots of Granzyme B⁺ CD4⁺ T cells in the injected tumors from WT and OX40^{-/-} mice after treatment with either MVAΔE5R-hFlt3L-mOX40L or PBS. FIG. 112B shows the graph of percentages of CD4⁺ T cells out of CD3⁺ cells. Data are means±SEM (n=4-10). FIG. 112C shows the graph of absolute numbers of CD4⁺ T cells per gram of tumor. Data are means±SEM (n=4-10). FIG. 112D shows the graph of percentages of Granzyme B⁺ CD4⁺ T cells out of CD4⁺ cells. Data are means±SEM (n=4-10). FIG. 112E shows the graph of absolute numbers of Granzyme B⁺ CD4⁺ T cells per gram of tumor. Data are means±SEM (n=4-10).

FIG. 113A shows the IT injection of MVAΔE5R-hFlt3L-mOX40L fails to reduce FoxP3⁺CD4⁺ T cells in the injected tumors from OX40^{-/-} mice. Representative dot plots of FoxP3⁺CD4⁺ T cells in the injected tumors from WT and OX40^{-/-} mice after treatment with either MVAΔE5R-hFlt3L-mOX40L or PBS. FIG. 113B shows the graph of percentages of FoxP3⁺CD4⁺ T cells out of CD4⁺ cells Data are means±SEM (n=4-10).

FIGS. 114A-115C demonstrate that IT injection of MVAΔE5R-hFlt3L-mOX40L reduces OX40⁺FoxP3⁺CD4⁺ and OX40⁺FoxP3⁻CD4⁺ T cells in the injected tumors from the WT mice. FIG. 114A shows the representative dot plots of OX40⁺FoxP3⁺CD4⁺ T cells in the injected tumors from WT and OX40^{-/-} mice after treatment with either MVAΔE5R-hFlt3L-mOX40L or PBS. FIG. 114B shows the graph of percentages of OX40⁺FoxP3⁺CD4⁺ T cells out of CD4⁺ cells. Data are means±SEM (n=4-10). FIG. 114C shows the graph of absolute numbers of OX40⁺FoxP3⁺CD4⁺ T cells per gram of tumor. Data are means±SEM (n=4-10).

FIG. 115A shows the representative dot plots of OX40⁺FoxP3⁻CD4⁺ T cells in the injected tumors from WT and OX40^{-/-} mice after treatment with either MVAΔE5R-hFlt3L-mOX40L or PBS. FIG. 115B shows the graph of percentages of OX40⁺FoxP3⁻CD4⁺ T cells out of CD4⁺ cells. Data are means±SEM (n=4-10). FIG. 115C shows the graph of absolute numbers of OX40⁺FoxP3⁻CD4⁺ T cells per gram of tumor. Data are means±SEM (n=4-10).

FIGS. 116A-119C are a series of graphical representations of data showing that intratumoral injection of MVAΔE5R-hFlt3L-mOX40L results in more proliferation and activation of tumor-infiltrating effector CD8⁺ and CD4⁺ T cells in distant non-injected tumors from OX40^{-/-} mice compared with WT mice. FIG. 116A shows the representative dot plots of Granzyme B⁺CD8⁺ T cells in non-injected tumors from WT and OX40^{-/-} mice after treatment with either MVAΔE5R-hFlt3L-mOX40L or PBS. FIG. 116B shows the graph of percentages of CD8⁺ T cells out of CD45⁺ cells. Data are means±SEM (n=5-10). FIG. 116C shows the graph of absolute numbers of CD8⁺ T cells per gram of tumor. Data are means±SEM (n=5-10). FIG. 116D shows the graph of percentages of Granzyme B⁺ CD8⁺ T cells out of CD8⁺ cells. Data are means±SEM (n=5-10). FIG. 116E shows the graph of absolute numbers of Granzyme B⁺ CD8⁺ T cells per gram of tumor. Data are means±SEM (n=5-10).

FIG. 117A shows the representative dot plots of Ki67⁺ CD8⁺ T cells in non-injected tumors from WT and OX40^{-/-} mice after treatment with either MVAΔE5R-hFlt3L-mOX40L or PBS. FIG. 117B shows the graph of percentages of Ki67⁺CD8⁺ T cells out of CD8⁺ cells. Data are means±SEM (n=5-10). FIG. 117C shows the graph of absolute numbers of Ki67⁺CD8⁺ T cells per gram of tumor. Data are means±SEM (n=5-10).

FIG. 118A shows the representative dot plots of Granzyme B⁺CD4⁺ T cells in none-injected tumors from WT and OX40^{-/-} mice after treatment with either MVAΔE5R-hFlt3L-mOX40L or PBS. FIG. 118B shows the graph of percentages of CD4⁺ T cells out of CD45⁺ cells. Data are means±SEM (n=5-10). FIG. 118C shows the graph of absolute numbers of CD4⁺ T cells per gram of tumor. Data are means±SEM (n=5-10). FIG. 118D shows the graph of percentages of Granzyme B⁺ CD4⁺ T cells out of CD4⁺ cells. Data are means±SEM (n=5-10). FIG. 118E shows the graph of absolute numbers of Granzyme B⁺ CD4⁺ T cells per gram of tumor. Data are means±SEM (n=5-10).

FIG. 119A shows the representative dot plots of Ki67⁺CD4⁺ T cells in non-injected tumors from WT and OX40^{-/-} mice after treatment with either MVAΔE5R-hFlt3L-mOX40L or PBS. FIG. 119B shows the graph of percentages of Ki67⁺CD4⁺ T cells out of CD4⁺ cells. Data are means±SEM (n=5-10). FIG. 119C shows the graph of absolute numbers of Ki67⁺CD4⁺ T cells per gram of tumor. Data are means±SEM (n=5-10).

FIGS. 120-124B are a series of graphical representations of data showing that intratumoral injection of MVAΔE5R-hFlt3L-mOX40L was capable of inducing antitumor effects without recruiting T cells from the lymphoid organs. FIG. 120 shows the experimental scheme. Briefly, B16-F10 melanoma cells were implanted intradermally to the left and right flanks of C57B/6J mice (5×10⁵ to the right flank and 2.5×10⁵ to the left flank). Nine days post tumor implantation, intratumoral injections (4×10⁷ pfu) of either MVAΔE5R-hFlt3L-mOX40L, or PBS were performed to the larger tumors on the right flank twice on day 9 and 12. The injected tumors were harvested at 2 days post second injection and tumor-infiltrating lymphocytes were analyzed by FACS. FTY720 (25 μg), which blocks egress of lymphocytes from the lymphoid organs, was given to the mice intraperitoneally on day 7, 9, 11, and 13.

FIG. 121 (upper panel) shows the graphs of tumor volumes of both injected and non-injected tumors over time. Data are means±SEM (n=7-8). FIG. 121 (lower panel) shows the graphs of tumor volumes of both injected and non-injected tumors at day 6 post first injection. Data are means±SEM (n=7-8).

FIGS. 122A-122D shows the representative dot plots of Granzyme B⁺ CD8⁺ T cells in injected tumors from mice after treatment with either MVAΔE5R-hFlt3L-mOX40L or PBS in combination with intraperitoneal delivery of FTY720 or DMSO. FIG. 122B shows the graph of percentages of CD8⁺ T cells out of CD45⁺ cells. Data are means±SEM (n=7-8). FIG. 122C shows the graph of percentages of Granzyme B⁺CD8⁺ T cells out of CD8⁺ cells. Data are means±SEM (n=7-8). FIG. 122D shows the graph of percentages of Ki67⁺CD8⁺ T cells out of CD8⁺ cells. Data are means±SEM (n=7-8).

FIG. 123A shows the representative dot plots of Granzyme B⁺ CD8⁺ T cells in TDLNs of the injected tumors from mice after treatment with either MVAΔE5R-hFlt3L-mOX40L or PBS in combination with intraperitoneal delivery of FTY720 or DMSO. FIG. 123B shows the graph of percentages of CD8⁺ T cells out of CD3⁺ cells. Data are means±SEM (n=7-8). FIG. 123C shows the graph of percentages of Granzyme B⁺CD8⁺ T cells out of CD8⁺ cells. Data are means±SEM (n=7-8).

FIG. 124A shows the representative dot plots of Ki67⁺ CD8⁺ T cells in TDLNs of the injected tumors from mice after treatment with either MVAΔE5R-hFlt3L-mOX40L or PBS in combination with intraperitoneal delivery of FTY720 or DMSO. FIG. 124B shows the graph of percentages of Ki67⁺CD8⁺ T cells out of CD8⁺ cells. Data are means±SEM (n=7-8).

FIGS. 125-129C are graphical representations of data showing intratumoral delivery of MVAΔE5R-hFlt3L-mOX40L delays tumor growth, activates CD8⁺ T cells and reduces FoxP3⁺CD4⁺ T cells in the injected tumors in a murine AT3 breast cancer fat pad implantation model. FIG. 125 is a scheme of tumor implantation and treatment for murine breast cancer AT3 fat pad implantation model. Briefly, AT3 cells (1×10⁶) were implanted into the₄th fat pad of the C57B/6J mice. 14 days post tumor implantation, intratumoral injections (6×10⁷ pfu) of MVAΔE5R-hFlt3L-mOX40L, or Heat-iMVA, or PBS, were performed twice, three days apart. The injected tumors were measured and harvested for FACS analysis.

FIG. 126A shows the graph of tumor volumes of injected AT3 tumors after treatment with MVAΔE5R-hFlt3L-mOX40L, or Heat-iMVA, or PBS over time. Data are means±SEM (n=5). Graph of tumor weight of injected tumors at day 6 post first injection. Data are means±SEM (n=5). FIG. 126B shows the tumor weighs on day 6. *=p<0.05.

FIG. 127A shows the representative dot plots of Granzyme B⁺ CD8⁺ T cells in injected AT3 tumors after treatment with either MVAΔE5R-hFlt3L-mOX40L, or Heat-iMVA, or PBS. FIG. 127B shows the graph of percentages of CD8⁺ T cells out of CD3⁺ cells. Data are means±SEM (n=5). FIG. 127C shows the graph of absolute numbers of CD8⁺ T cells per gram of tumor. Data are means±SEM (n=5). FIG. 127D shows the graph of percentages of Granzyme B⁺ CD8⁺ T cells out of CD8⁺ cells. Data are means±SEM (n=5). FIG. 127E shows the graph of absolute numbers of Granzyme B⁺ CD8⁺ T cells per gram of tumor. Data are means±SEM (n=5).

FIG. 128A shows the representative dot plots of Granzyme B⁺ CD4⁺ T cells in injected AT3 tumors after treatment with either MVAΔE5R-hFlt3L-mOX40L, or Heat-iMVA, or PBS. FIG. 128B shows the graph of percentages of CD4⁺ T cells out of CD3⁺ cells. Data are means±SEM (n=5). FIG. 128C shows the graph of absolute numbers of CD4⁺ T cells per gram of tumor. Data are means±SEM (n=5). FIG. 128D shows the graph of percentages of Granzyme B⁺ CD4⁺ T cells out of CD4⁺ cells. Data are means±SEM (n=5). FIG. 128E shows the graph of absolute numbers of Granzyme B⁺ CD4⁺ T cells per gram of tumor. Data are means±SEM (n=5).

FIG. 129A shows the representative dot plots of FoxP3⁺ CD4⁺ T cells in injected AT3 tumors after treatment with either MVAΔE5R-hFlt3L-mOX40L, or Heat-iMVA, or PBS. FIG. 129B shows the graph of percentages of FoxP3⁺CD4⁺ T cells out of CD4⁺ cells. Data are means±SEM (n=5). FIG. 129C shows the graph of absolute numbers of FoxP3⁺CD4⁺ T cells per gram of tumor. Data are means±SEM (n=5).

FIGS. 130-131B are graphical representations of data showing that combination of IT delivery of MVAΔE5R-hFlt3L-mOX40L and intraperitoneal delivery of anti-PD-L1 results in enhanced therapeutic efficacy in a bilateral B16-F10 melanoma implantation model.

FIG. 130 shows the experimental scheme. Briefly, B16-F10 melanoma cells were implanted intradermally to the left and right flanks of C57B/6J mice (5×10⁵ to the right flank and 1×10⁵ to the left flank). Seven days post tumor implantation, 4×10⁷ pfu of either MVAΔE5R-hFlt3L-mOX40L or PBS was intratumorally (IT) injected into the larger tumors on the right flank twice per week. One group of the mice also received anti-PD-L1 antibody (250 μg) twice a week in conjunction with IT MVAΔE5R-hFlt3L-mOX40L. Tumor volumes and mice survival were monitored.

FIG. 131A shows tumor volumes of both injected and non-injected tumors in mice treated with either PBS or MVAΔE5R-hFlt3L-mOX40L intratumorally, or with the combination of IT MVAΔE5R-hFlt3L-mOX40L plus IP anti-PD-L1. FIG. 131B shows the Kaplan Meier survival curve of the three groups.

FIGS. 132A-134B are graphical representations of data showing MVAΔE5R-hFlt3L-hOX40L infection of BMDCs induces IFNB gene expression and IFN-□ protein secretion; infection of human tumors (extramammary Paget's disease) with MVAΔE5R-hFlt3L-hOX40L ex vivo results in the increase of Granzyme B⁺ CD8⁺ T cells and the reduction of FoxP3⁺CD4⁺ T cells.

FIG. 132A shows the RT-PCR results of BMDCs that were infected with either MVA or MVAΔE5R-hFlt3L-hOX40L at a MOI of 10. Cells were collected at 6 h post infection. RNAs were extracted and RT-PCRs were performed. FIG. 132B shows the IFN-β protein levels in BMDCs infected with either MVA or MVAΔE5R-hFlt3L-hOX40L at a MOI of 10. Supernatants were collected at 19 h post infection. IFN-β protein levels were determined by ELISA.

FIG. 133A shows the representative dot plots of Granzyme B⁺ CD8⁺ T cells in human tumors after infection with MVAΔE5R-hFlt3L-mOX40L or PBS for two days. FIG. 133B shows the representative dot plots of FoxP3⁺CD4⁺ T cells in human tumors after infection with MVAΔE5R-hFlt3L-mOX40L or PBS for two days.

FIG. 134A shows the graph of percentages of Granzyme⁺CD8⁺ T cells out of CD8⁺ cells after infection with MVAΔE5R-hFlt3L-mOX40L or PBS control for two days. Data are means±SEM (n=3). FIG. 134B shows the graph of percentages of FoxP3⁺CD4⁺ T cells T cells out of CD4⁺ cells after infection with MVAΔE5R-hFlt3L-mOX40L or PBS control for two days. Data are means±SEM (n=3).

FIGS. 135A-137B are graphical representations of data showing MVAΔE3LΔE5R induces higher levels of type I IFN in BMDCs and B16-F10 melanoma cells compared with MVAΔE5R or MVAΔE3L.

FIGS. 135A-135C show a scheme of generating recombinant MVAΔE3LΔE5R and MVAΔE3LΔE5R-hFlt3L-mOX40L through homologous recombination at the E2L and E4L loci of the MVAΔE5R or MVAΔE5R-hFlt3L-mOX40L genome. Homologous recombination that occurred at the E2L and E4L loci results in the deletion of E3L gene from the MVAΔE5R or MVAΔE5R-hFlt3L-mOX40L genome.

FIG. 136 shows that MVAΔE3LΔE5R infection of BMDCs induced higher levels of IFNB gene expression compared with MVAΔE5R, MVA, or Heat-iMVA. BMDCs from WT or cGAS^{-/-} mice were infected with MVA, Heat-iMVA, MVAΔE5R, or MVAΔE3LΔE5R at a MOI of 10. Cells were collected at 6 h post infection and RNAs were extracted. Quantitative RT-PCR analyses were performed to examine the expression of IFNB gene.

FIGS. 137A-137B show that MVAΔE3LΔE5R infection of murine B16-F10 melanoma cells induces higher levels of IFNB gene expression and IFN-Q protein secretion compared with MVAΔE3L or MVAΔE5R. FIG. 137A shows the quantitative RT-PCR analyses with WT or MDA5^{-/-}, or STING^{-/-}MDA5^{-/-} B16-F10 cells infected with MVAΔE3L, or MVAΔE5R or MVAΔE3LΔE5R at a MOI of 10. Cells were collected at 16 h post infection and RNAs were extracted. Quantitative RT-PCR analyses were performed to examine the expression of IFNB gene. FIG. 137B shows the IFN-□ protein levels in WT or MDA5^{-/-}, or STING^{-/-}MDA5^{-/-} B16-F10 cells infected with MVAΔE3L, or MVAΔE5R, or MVAΔE3LΔE5R at a MOI of 10. Supernatants were collected at 24 h post infection and IFN-D protein levels in the supernatants were determined by ELISA.

FIGS. 138-139B are graphical representations of data showing MVAΔE3LΔE5R-hFlt3L-mOX40L expressed human Flt3L and murine OX40L transgenes in B16-F10 melanoma cells and intratumoral delivery of MVAΔE3LΔE5R-hFlt3L-mOX40L induced stronger systemic antitumor T cell responses in a B16-F10 murine melanoma model.

FIG. 138 shows the FACS data demonstrating the mOX40L and hFlt3L expression on B16-F10 cells infected with either MVAΔE5R-hFlt3L-mOX40L or with MVAΔE3LΔE5R-hFlt3L-mOX40L. B16-F10 cells were infected with either MVAΔE5R-hFlt3L-mOX40L, or with MVAΔE3LΔE5R-hFlt3L-mOX40L, or with MVAΔE3LΔE5R at a MOI of 10. Cells were washed 1 h later and harvested at 24 h post infection. Cells were strained with anti-mOX40L or anti-hFlt3L antibody for FACS.

FIGS. 139A-139B shows that intratumoral injection of MVAΔE3LΔE5R-hFlt3L-mOX40L generated stronger antitumor-specific T cells in the spleens compared with MVAΔE5R-hFlt3L-mOX40L. Briefly, B16-F10 melanoma cells were implanted intradermally to the left and right flanks of C57B/6J mice (5×10⁵ to the right flank and 2.5×10⁵ to the left flank). Seven days post tumor implantation, 2×10⁷ pfu of either MVAΔE5R-hFlt3L-mOX40L, MVAΔE3LΔE5R-hFlt3L-mOX40L, an equivalent amount of Heat-iMVA, or PBS was intratumorally (IT) injected into the larger tumors on the right flank twice, three days apart. Spleens were harvested at 2 days post second injection, ELISPOT analyses were performed to evaluate tumor-specific T cells in the spleens. ELISPOT assay was performed by co-culturing irradiated B16-F10 cells (150,000) and splenocytes (1,000,000) in a 96-well plate. FIG. 139A shows the image of ELISPOT of triplicate samples of combined splenocytes from mice in the same treatment group. FIG. 139B shows the graph of IFN-γ⁺ spots per 1,000,000 splenocytes. Each dot represents splenocyte samples from an individual mouse (n=5-6) (*P<0.05; **P<0.01, t test).

FIGS. 140-141B are graphical representations of data showing intratumoral delivery of MVAΔE3LΔE5R-hFlt3L-mOX40L delayed the growth of both WT and □2M^{-/-} B16-F10 tumor cells.

FIG. 140 shows the experimental scheme. Briefly, WT or b2M^{-/-} B16-F10 tumor cells (2×10⁵) (which were generated by CRISPR-cas9 technology in the inventors' lab) were implanted intradermally to the right flanks of C57BL/6J mice. 10 days after tumor implantation, tumors were injected with MVAΔE3LΔE5R-hFlt3L-mOX40L twice a week. Tumor volumes were measured and mice survival were monitored.

FIG. 141A shows tumor volumes of the injected WT and b2M^{-/-} B16-F10 tumors in mice treated with either PBS or MVAΔE5R-hFlt3L-mOX40L intratumorally. FIG. 141B shows the Kaplan Meier survival curve of the four groups.

FIGS. 142-148 are a series of graphical representations of data showing that intratumoral injection of MVAΔE3LΔE5R-hFlt3L-mOX40L generated more activated tumor-infiltrating effector T cells and reduced percentage of macrophage and DCs in injected tumors compared with MVAΔE5R-hFlt3L-mOX40L in a AT3 bilateral tumor implantation model.

FIG. 142 shows the experimental scheme. Briefly, 10⁵ AT3 breast cancer cells were implanted into the 4^{th} fat pad of the C57B/6J mice. Twelve days post tumor implantation, 6×10⁷ pfu of either MVAΔE5R-hFlt3L-mOX40L, MVAΔE3LΔE5R-hFlt3L-mOX40L, or PBS was intratumorally (IT) injected into the tumors on both flanks twice, three days apart. Injected tumors were isolated. Tumor infiltrating lymphocytes and myeloid cells were analyzed by FACS.

FIG. 143A shows the representative dot plots of Granzyme B⁺ CD8⁺ T cells in injected tumors after treatment with either MVAΔE5R-hFlt3L-mOX40L, or MVAΔE3LΔE5R-hFlt3L-mOX40L. FIG. 143B shows the graph of percentages of CD8⁺ T cells out of CD45⁺ cells. Data are means±SEM (n=4-6). (**P<0.01; ***P<0.001, t test). FIG. 143C shows the graph of absolute number of CD8⁺ T cells. Data are means±SEM (n=4-6). (**P<0.01; ***P<0.001, t test). FIG. 143D shows the graph of percentages of Granzyme B⁺ CD8⁺ T cells out of CD8⁺ cells. Data are means±SEM (n=4-6) (**P<0.01; ***P<0.001, t test). FIG. 143E shows the graph of absolute number of Granzyme B⁺ CD8⁺ T cells. Data are means±SEM (n=4-6) (**P<0.01; ***P<0.001, t test).

FIG. 144A shows the representative dot plots of Ki67⁺ CD8⁺ T cells in injected tumors after treatment with either MVAΔE5R-hFlt3L-mOX40L or MVAΔE3LΔE5R-hFlt3L-mOX40L. FIG. 144B shows the graph of percentages of Ki67⁺ CD8⁺ T cells out of CD8⁺ cells Data are means±SEM (n=4-6) (**P<0.01; ***P<0.001, t test). FIG. 144C shows the graph of absolute number of Ki67⁺ CD8⁺ T cells. Data are means±SEM (n=4-6) (**P<0.01; ***P<0.001, t test).

FIG. 145A shows the representative dot plots of Granzyme B⁺ CD4⁺ T cells in injected tumors after treatment with either MVAΔE5R-hFlt3L-mOX40L or MVAΔE3LΔE5R-hFlt3L-mOX40L. FIG. 145B shows the graph of percentages of CD4⁺ T cells out of CD3⁺ cells. Data are means±SEM (n=4-6). (**P<0.01; ***P<0.001, t test). FIG. 145C shows the graph of absolute number of CD4⁺ T cells. Data are means±SEM (n=4-6). (**P<0.01; ***P<0.001, t test). FIG. 145D shows the graph of percentages of Granzyme B⁺ CD4⁺ T cells out of CD4⁺ cells. Data are means±SEM (n=4-6) (**P<0.01; ***P<0.001, t test). FIG. 145E shows the graph of absolute number of Granzyme B⁺ CD4⁺ T cells. Data are means □T cells. Data aP<0.01; ***P<0.001, t test).

FIG. 146A shows the representative dot plots of FoxP3⁺CD4⁺ T cells in injected tumors after treatment with either MVAΔE5R-hFlt3L-mOX40L or MVAΔE3LΔE5R-hFlt3L-mOX40L. FIG. 146B shows the graph of percentages of FoxP3⁺CD4⁺ T cells out of CD4⁺=cells. Data are means±SEM (n=4-6). (**P<0.01; ***P<0.001, t test). FIG. 146C shows the graph of absolute number of FoxP3⁺CD4⁺ T cells. Data are means±SEM. Data are P<0.01; ***P<0.001, t test).

FIG. 147A shows the representative dot plots of OX40⁺FoxP3⁺ CD4⁺ T cells in injected tumors after treatment with either MVAΔE5R-hFlt3L-mOX40L or MVAΔE3LΔE5R-hFlt3L-mOX40L. FIG. 147B shows the graph of percentages of OX40⁺FoxP3⁺CD4⁺ T cells out of FoxP3⁺CD4⁺ cells. Data are means±SEM (n=4-6). (**P<0.01; ***P<0.001, t test). FIG. 147C shows the graph of absolute number of OX40⁺FoxP3⁺CD4⁺ T cells. Data are means±SEM (n=4-6). (**P<0.01; ***P<0.001, t test).

FIGS. 148A-148H are series of data showing that intratumoral injection of MVAΔE5R-hFlt3L-mOX40L or MVAΔE3LΔE5R-hFlt3L-mOX40L reduces the percentage of macrophages and DCs in injected tumors. FIG. 148A shows the graph of percentages of macrophages in injected tumors after treatment with either MVAΔE5R-hFlt3L-mOX40L or MVAΔE3LΔE5R-hFlt3L-mOX40L. Data are means±SEM (n=4-6). FIG. 148B shows the graph of absolute number of macrophages. Data are means±SEM (n=4-6). (**P<0.01; ***P<0.001, t test). FIG. 148C shows the graph of percentages of DCs. Data are means±SEM (n=4-6). (**P<0.01; ***P<0.001, t test). FIG. 148D shows the graph of absolute number of DCs. Data are means±SEM (n=4-6). (**P<0.01; ***P<0.001, t test). FIG. 148E shows the graph of percentages of CD11b⁺DCs. Data are means±SEM (n=4-6). (**P<0.01; ***P<0.001, t test). FIG. 148F shows the graph of absolute number of CD11b⁺DCs. Data are means±SEM (n=4-6). (**P<0.01; ***P<0.001, t test). FIG. 148G shows the graph of percentages of CD103⁺DCs. Data are means±SEM (n=4-6). (**P<0.01; ***P<0.001, t test). FIG. 148H shows the graph of absolute number of CD103⁺DCs. Data are means±SEM (n=4-6). (**P<0.01; ***P<0.001, t test).

FIGS. 149-150 are a series of graphical representations of data showing that the combination of intratumoral injection of MVAΔE3LΔE5R-hFlt3L-mOX40L with anti-PD-L1 and anti-CTLA-4 antibody had superior anti-tumor efficacy in MMTV-PyMT breast cancer model. FIG. 149 shows the experimental scheme. After the first tumor became palpable, 4×10⁷ pfu of MVAΔE3LΔE5R-hFlt3L-mOX40L or PBS was intratumorally (IT) injected into the tumors twice a week. 250 μg Anti-PD-L1 and 100 μg anti-CTLA-4 antibody or isotype control antibody were injected intraperitoneally to each mouse twice a week. Tumor volumes were measured twice a week. FIG. 150 shows the graph of tumor growth curve after treatment with IT MVAΔE3LΔE5R-hFlt3L-mOX40L and IP anti-PD-L1 and anti-CTLA-4 antibody. Data are means±SEM (n=3-4).

FIGS. 151-152B are a series of graphical representations of data showing that deletion of C11R gene from MVAΔE5R-hFlt3L-mOX40L increases IFN production in BMDCs.

FIG. 151 is a schematic diagram of homologous recombination to generate MVAΔE5R-hFlt3L-mOX40AC11R.

FIGS. 152A-152B show that MVAΔE5R-hFlt3L-mOX40AC11R infection of BMDCs induces higher levels of IFNB gene expression (FIG. 152A) and protein secretion (FIG. 152B) compared with MVAΔE5R or MVA. BMDCs from WT mice were infected with MVA, MVAΔE5R, or MVAΔE5R-hFlt3L-mOX40ΔC11R at a MOI of 10. For assessing IFNB gene expression, cells were collected at 6 h post infection and RNAs were extracted. Quantitative RT-PCR analyses were performed to examine the expression of IFNB gene. For testing IFN-b protein secretion from BMDCs, supernatants were collected at 19 h post infection. IFN-b protein levels were determined by ELISA.

FIGS. 153-154C are a series of graphical representations of data showing that deletion of WR199 gene from MVA or MVAΔE5R-hFlt3L-mOX40L increases IFN production in BMDCs.

FIG. 153 is a schematic diagram of homologous recombination to generate MVAΔE5R-hFlt3L-mOX40ΔWR199. Homologous recombination that occurred at the B17L and B19R loci results in the deletion of WR199 and the insertion of expression cassette for mcherry flanked by two FRT sites.

FIG. 154A shows the IFNB gene expression in PBS, MVA, MVAΔWR199, or Heat-iMVA infected BMDCs from WT or cGAS^{-/-} mice at a MOI of 10. FIGS. 154B-154C shows that MVAΔE5R-hFlt3L-mOX40ΔC11R infection of BMDCs induces higher levels of IFNB gene expression (FIG. 154B) and protein secretion (FIG. 154C) compared with MVAΔE5R or MVA. BMDCs from WT mice were infected with MVA, MVAΔE5R, or MVAΔE5R-hFlt3L-mOX40ΔC11R at a MOI of 10. For assessing IFNB gene expression, cells were collected at 6 h post infection and RNAs were extracted. Quantitative RT-PCR analyses were performed to examine the expression of IFNB gene. For testing IFN-□ protein secretion from BMDCs, supernatants were collected at 19 h post infection. IFN-□ protein levels were determined by ELISA.

FIG. 155 shows a scheme of generating recombinant VACVΔB2R virus through homologous recombination at the B1R and B3R loci of the vaccinia virus (WR) genome. Homologous recombination that occurred at the B1R and B3R loci results in the deletion of B2R gene from the vaccinia virus (WR) genome.

FIGS. 156A-156B show that VACVΔB2R was highly attenuated in an intranasal infection model. FIG. 156A shows the weight loss after intranasal infection with either high dose of VACVΔB2R (H, 2×10⁷ pfu), or low dose of VACVΔB2R (L, 2×10⁶ pfu). FIG. 156B shows the Kaplan-Meier survival curves of intranasal infected mice with high dose of VACVΔB2R (H, 2×10⁷ pfu) or low dose of VACVΔB2R (L, 2×10⁶ pfu).

FIGS. 157A-157B shows a schematic diagrams of generating recombinant VACVΔE3L83NΔB2R, VACVΔE5RΔB2R, and VACVΔE3L83NΔE5RΔB2R viruses. FIG. 157A shows that VACVΔE3L83NΔB2R was generated through homologous recombination at the B1R and B3R loci of the vaccinia VACVΔE3L83N genome, resulting in the deletion of B2R gene from the VACVΔE3L83N genome. FIG. 157B shows that VACVΔE5RΔB2R and VACVΔE3L83NΔE5RΔB2R are generated through homologous recombination at the B1R and B3R loci of the vaccinia VACVΔE5R or VACVΔE3L83NΔE5R genome, respectively. Homologous recombination that occurred at the B1R and B3R loci results in the deletion of B2R gene from the VACVΔE5R or VACVΔE3L83NΔE5R genome, respectively.

FIG. 158 shows that VACVΔE3L83NΔE5R, VACVΔE5RΔB2R, and VACVΔE3L83NΔE5RΔB2R are highly attenuated in an intranasal infection model. WT mice were intranasally infected with 2×10⁷ pfu of VACVΔB2R, VACVΔE5R, VACVΔE3L83NΔE5R, VACVΔE5RΔB2R, or VACVΔE3L83NΔE5RΔB2R, and survival and weight loss were monitored daily. This figure shows weight loss after intranasal infection with these five different viruses.

FIGS. 159A-159B shows that VACVΔE5RΔB2R and VACVΔE3L83NΔE5RΔB2R infection of murine BMDC induce higher levels of IFNB gene expression and IFN-γ protein secretion compared with. VACVΔB2R or VACVΔE5R. FIG. 159A shows the IFNB gene expression in WT and cGAS knockout BMDC cells infected with VACV, VACVΔ83N, VACVΔB2R, VACVΔE5R, VACVΔE5RΔB2R, VACVΔE3L83NΔE5R, or VACVΔE3L83NΔE5RΔB2R at a MOI of 10. Cells were collected at 6 hours post infection and RNAs were extracted. Quantitative RT-PCR analyses were performed to examine the expression of IFNB gene. FIG. 159B shows the IFN-γ protein secretion by WT and cGAS knockout BMDC cells infected with VACV, VACVΔ83N, VACVΔB2R, VACVΔE5R, VACVΔE5RΔB2R, VACVΔE3L83NΔE5R, or VACVΔE3L83NΔE5RΔB2R at a MOI of 10. Supernatants were collected at 24 h post infection and IFN-γ protein levels in the supernatants were determined by ELISA.

FIG. 160 shows that VACVΔE5RΔB2R and VACVΔE3L83NΔE5RΔB2R infection of murine BMDC induce higher levels of phosphorylation of STING, IRF3 and TBK1 compared with VACVΔB2R or VACVΔE5R. WT BMDC cells were infected with VACV, VACVΔB2R, VACVΔE5R, VACVΔE5RΔB2R, VACVΔE3L83NΔE5R, or VACVΔE3L83NΔE5RΔB2R at a MOI of 10. Cell lysis were collected at different time points. The phosphorylation of STING, IRF3, and TBK1 were detected by antibodies against phosphorylated STING, IRF3, and TBK1, respectively.

FIG. 161 shows a scheme of stepwise strategy to generate recombinant VACVΔE3L83NΔTKΔE5R virus expressing anti-muCTLA-4 antibody, and hFlt3L, mOX40L and mIL12 proteins through homologous recombination first at the TK and then at the E5R loci of the VACVΔE3L83N genome. pCB vector was used to insert a single expression cassette to express the anti-muCTLA-4 antibody heavy and light chains under the control of the vaccinia virus synthetic early and late promoter (PsE/L). Homologous recombination that occurred at the TK-L and TK-R sites results in the insertion of expression cassette of anti-muCTLA-4 antibody into TK locus on VACVΔE3L83N genome. pUC57 vector was used to insert two expression cassettes designed to express both hFlt3L-mOX40L fusion protein and mIL-12 using the vaccinia viral synthetic early and late promoter (PsE/L). The coding sequence of the hFlt3L-mOX40L was separated by a furin cleavage site followed by a Pep2A sequence. The coding sequence of p40 and p30 subunits of mIL12 was separated by a furin cleavage site followed by a Pep2A sequence. The C-terminus of p30 subunit was tagged with a matrix binding sequence. Homologous recombination at the E4L and E6R loci results in the insertion of expression cassette for hFlt3L-mOX40L and mIL12 into the E5L locus of VACVΔE3L83N-ΔTK-anti-muCTLA-4 genome.

FIG. 162 shows a scheme of generating recombinant VACVΔE3L83N-ΔTK-anti-muCTLA-4-ΔE5R-hFlt3L-mOX40L-mIL-12 virus with deletion of B2R gene through homologous recombination at the B1R and B3R loci of the VACVΔE3L83N-ΔTK-anti-muCTLA-4-ΔE5R-hFlt3L-mOX40L-mIL-12 (OV-VACVΔE5R) genome. Homologous recombination that occurred at the B1R and B3R loci results in the deletion of B2R gene from the virus genome to generate the VACVΔE3L83N-ΔTK-anti-muCTLA-4ΔE5R-hFlt3L-mOX40L-mIL-12-ΔB2R virus (OV-VACVΔE5RΔB2R).

FIG. 163 shows a multistep growth curve of the recombinant viruses VACVΔE3L83N-ΔTK-anti-muCTLA-4ΔE5R-hFlt3L-mOX40L-mIL-12 (OV-VACVΔE5R) and VACVΔE3L83N-ΔTK-anti-muCTLA-4ΔE5R-hFlt3L-mOX40L-mIL-12-ΔB2R (OV-VACVΔE5RΔB2R) in BSC40 cells compared with WT VACV. BSC40 cells were infected with VACV, VACVΔE3L83N-ΔTK-anti-muCTLA-4ΔE5R-hFlt3L-mOX40L-mIL-12, and VACVΔE3L83N-ΔTK-anti-muCTLA-4ΔE5R-hFlt3L-mOX40L-mIL-12-ΔB2R at a MOI of 0.01. Virus samples were collected at different time points and virus titers were determined using BSC40 cells.

FIGS. 164A-164B shows that VACVΔE3L83N-ΔTK-anti-muCTLA-4ΔE5R-hFlt3L-mOX40L-mIL-12-ΔB2R (OV-VACVΔE5RΔB2R) infection of murine BMDC induce higher levels of IFNB gene expression and IFN-γ protein secretion compared with VACVΔE3L83N-ΔTK-anti-muCTLA-4ΔE5R-hFlt3L-mOX40L-mIL-12 (OV-VACVΔE5R). FIG. 164A shows the IFNB gene expression in WT BMDC cells infected with VACVΔE3L83N-ΔTK-anti-muCTLA-4ΔE5R-hFlt3L-mOX40L-mIL-12, VACVΔE3L83N-ΔTK-anti-muCTLA-4ΔE5R-hFlt3L-mOX40L-mIL-12-ΔB2R, or Heat-iMVA at a MOI of 10. Cells were collected at 6 hours post infection and RNAs were extracted. Quantitative RT-PCR analyses were performed to examine the expression of IFNB gene. FIG. 164B shows the IFN-γ protein secretion in same infection as in FIG. 164A. Supernatants were collected at 24 h post infection and IFN-γ protein levels in the supernatants were determined by ELISA.

FIGS. 165A-165C show the expression of the transgenes anti-muCTLA-4, mOX40L, or human Flt3L in VACVΔE3L83N-ΔTK-anti-muCTLA-4-ΔE5R-hFlt3L-mOX40L-mIL-12 (OV-VACVΔE5R) infected B16-F10 cells and in tumors injected with virus. FIG. 165A shows a western blot demonstrating that murine anti-CTLA-4 and human Flt3L are expressed in VACVΔE3L83N-ΔTK-anti-muCTLA-4-ΔE5R-hFlt3L-mOX40L-mIL-12 (OV-VACVΔE5R)-infected B16-F10 cells. FL: full length of anti-muCTLA-4; HC: heavy chain of anti-muCTLA-4; LC: light chain of anti-muCTLA-4. FIG. 165B shows a dot plot of FACS analysis of mOX40 expression on murine melanoma cells B16-F10. Cells were infected with VACVΔE3L83N-ΔTK-anti-muCTLA-4-ΔE5R-hFlt3L-mOX40L-mIL-12 (expressing GFP) at a MOI of 10 for 24 h. No virus mock infection control was included. Cells were stained with anti-mOX40L antibody. FIG. 165C shows the mIL-12 expression in B16-F10 melanoma tumors after intratumoral injection of VACVΔE3L83N-ΔTK-anti-muCTLA-4-ΔE5R-hFlt3L-mOX40L-mIL-12 (OV-VACVΔE5R) virus. Intradermally implanted B16-F10 melanoma tumors were injected with 4×10⁷ pfu of VACVΔE3L83N-ΔTK-anti-muCTLA-4-ΔE5R-hFlt3L-mOX40L-mIL-12 (OV-VACVΔE5R) in 100 μl of PBS, and tumors were collected at 48 hours after treatment. Tumor samples were lysed and the expression of murine IL-12 were examined by western blot using anti-p40 antibody.

FIGS. 166A-166D. FIGS. 166A-166C show the expression and secretion of murine IL-12 after VACVΔE3L83N-ΔTK-anti-muCTLA-4-ΔE5R-hFlt3L-mOX40L-mIL-12 (OV-VACVΔE5R) virus infection of three different murine cancer cell lines. Tumor cells were infected with VACV, VACVΔE3L83N-ΔTK-anti-muCTLA-4-ΔE5R-hFlt3L-mOX40L-mIL-12, or mock infected. Supernatant were collected at 24 and 48 hours after virus infection and the concentration of IL-12 in cell culture supernatant were determined by ELISA. FIG. 166A shows the mIL-12 levels in OV-VACVΔE5R-infected B16-F10 melanoma cells. FIG. 166B shows the mIL-12 levels in OV-VACVΔE5R-infected 4T1 breast cancer cells. FIG. 166C shows the mIL-12 level in OV-VACVΔE5R-infected MC38 colon cancer cells. FIG. 166D shows serum mIL-12 levels in mice treated with OV-VACVΔE5R. At 48 h and 72 h post intratumoral injection of the virus, mice were euthanized and blood/serum was collected for cytokine measurement by ELISA.

FIGS. 167A-167B shows antitumor efficacy of intratumoral delivery of VACVΔE3L83N-ΔTK-anti-muCTLA-4-ΔE5R-hFlt3L-mOX40L-mIL-12 (OV-VACVΔE5R) either alone or in combination with anti-PD-L1 antibody in a bilateral B16-F10 tumor implantation model. Briefly, B16-F10 melanoma cells were implanted intradermally to the left and right flanks of C57B/6J mice (5×10⁵ to the right flank and 1×10⁵ to the left flank). Seven days post tumor implantation, 4×10⁷ pfu of either VACVΔE3L83N-ΔTK-anti-muCTLA-4-ΔE5R-hFlt3L-mOX40L-mIL-12 (OV-VACVΔE5R), Heat-iMVA, or PBS was intratumorally (IT) injected into the larger tumors on the right flank twice per week. One group of the mice also received anti-PD-L1 antibody (250 μg) twice a week in conjunction with IT VACVΔE3L83N-ΔTK-anti-muCTLA-4-ΔE5R-hFlt3L-mOX40L-mIL-12 (OV-VACVΔE5R). Tumor volumes and mice survival were monitored. FIG. 167A shows tumor volumes of both injected and non-injected tumors in mice treated with either PBS or VACVΔE3L83N-ΔTK-anti-muCTLA-4-ΔE5R-hFlt3L-mOX40L-mIL-12 (OV-VACVΔE5R), Heat-iMVA intratumorally, or with the combination of IT VACVΔE3L83N-ΔTK-anti-muCTLA-4-ΔE5R-hFlt3L-mOX40L-mIL-12 (OV-VACVΔE5R) plus IP anti-PD-L1. FIG. 167B shows the Kaplan Meier survival curve of the four groups.

FIGS. 168A-168B show that intratumoral injection of VACVΔE3L83N-ΔTK-anti-muCTLA-4-ΔE5R-hFlt3L-mOX40L-mIL-12ΔB2R (OV-VACVΔE5RΔB2R) generated stronger antitumor-specific T cells in the spleens compared with VACVΔE3L83N-ΔTK-anti-muCTLA-4-ΔE5R-hFlt3L-mOX40L-mIL-12 (OV-VACVΔE5R) or Heat-iMVA. Briefly, B16-F10 melanoma cells were implanted intradermally to the left and right flanks of C57B/6J mice (5×10⁵ to the right flank and 2.5×10⁵ to the left flank). Seven days post tumor implantation, 4×10⁷ pfu of either OV-VACVΔE5RΔB2R, OV-VACVΔE5R, an equivalent amount of Heat-iMVA, or PBS was intratumorally (IT) injected into the larger tumors on the right flank twice, three days apart. Spleens were harvested at 2 days post second injection, ELISPOT analyses were performed to evaluate tumor-specific T cells in the spleens. ELISPOT assay was performed by co-culturing irradiated B16-F10 cells (150,000) and splenocytes (1,000,000) in a 96-well plate. FIG. 168A: Graph of IFN-γ⁺ spots per 1,000,000 splenocytes. Each dot represents splenocytes from an individual mouse (n=4) (*P<0.05; **P<0.01, ****P<0.0001, t test). FIG. 168B: Image of ELISPOT of triplicate samples of combined splenocytes from mice in the same treatment group.

FIG. 169 shows a scheme of stepwise strategy to generate recombinant VACVΔE3L83NΔTKΔE5R virus expressing anti-huCTLA-4 antibody, and hFlt3L, hOX40L and hIL12 proteins through homologous recombination first at the TK and then at the E5R loci of the VACVΔE3L83N genome. pCB vector was used to insert a single expression cassette to express the anti-huCTLA-4 antibody heavy and light chains under the control of the vaccinia virus synthetic early and late promoter (PsE/L). Homologous recombination that occurred at the TK-L and TK-R sites results in the insertion of expression cassette of anti-huCTLA-4 antibody into TK locus on VACVΔE3L83N genome, which was generated by deleting the DNA fragment of E3L gene encoding N-terminal 83 amino acid. pUC57 vector was used to insert two expression cassettes designed to express both hFlt3L-hOX40L fusion protein and mIL-12 using the vaccinia viral synthetic early and late promoter (PsE/L). The coding sequence of the hFlt3L-mOX40L was separated by a furin cleavage site followed by a Pep2A sequence. The coding sequence of p40 and p30 subunits of hIL12 was separated by a furin cleavage site followed by a Pep2A sequence. The C-terminus of p30 subunit was tagged with a matrix binding sequence. Homologous recombination at the E4L and E6R loci results in the insertion of expression cassette for hFlt3L-hOX40L and hIL12 into the E5L locus of VACVΔE3L83N-ΔTK-anti-muCTLA-4 genome.

FIG. 170 shows a scheme of generating recombinant myxoma virus (Lausanne strain) with deletion of M063R gene through homologous recombination at the M062R and M064R loci of the myxomaΔM127-mcherry genome, as well as generating recombinant myxoma virus (Lausanne strain) with deletion of M064R gene through homologous recombination at the M063R and M065R loci of the myxomaΔM127-mcherry genome. Homologous recombination that occurred at the M062R and M064R loci results in the deletion of M063R gene from the virus genome to generate MyxomaΔM063R virus. Homologous recombination that occurred at the M063R and M065R loci results in the deletion of M064R gene from the virus genome to generate MyxomaΔM064R virus.

FIGS. 171A-171B show that MyxomaΔM064R and MyxomaΔM063R infection of murine BMDC induce higher levels of IFNB gene expression and IFN-B protein secretion compared with the parental myxoma virus expressing mcherry (Myxoma-mcherry) which also contains a deletion of the M0127 gene. BMDC cells were infected with Myxoma-mcherry, MyxomaΔM063R, MyxomaΔM064R, or MVA at a MOI of 10. Cells were collected at 6 hours post infection and RNAs were extracted. Quantitative RT-PCR analyses were performed to examine the expression of IFNB gene. FIG. 171A shows the RT-PCR result of IFNB gene expression in infected BMDCs. Supernatants were collected at 24 h post infection and IFN-β protein levels in the supernatants were determined by ELISA. FIG. 171B shows the ELISA results of IFN-β protein levels in the supernatants of infected BMDCs.

FIGS. 172-174B are a series of graphical representations of data showing that intratumoral injection of myxomaΔM064R or myxoma-mcherry leads to activation of effector CD4⁺ and CD8⁺ T cells in a B16-F10 melanoma model. FIG. 172 shows the experimental scheme. Briefly, B16-F10 melanoma cells were implanted intradermally to the left and right flanks of C57B/6J mice (5×10⁵ to the right flank and 2.5×10⁵ to the left flank). Seven days post tumor implantation, 2×10⁷ pfu of either Myxoma-mCherry, MyxomaΔM064R, MVAΔE5R or PBS was intratumorally (IT) injected into the larger tumors on the right flank. Two days post injection, the injected tumors were isolated and tumor infiltrating lymphocytes were analyzed by FACS. FIG. 172 shows that intratumoral injection of myxomaΔ0M64R or myxoma-mcherry leads to activation of effector CD4⁺ and CD8⁺ T cells in a B16-F10 melanoma model.

FIG. 173A shows the representative dot plots of Granzyme B⁺ CD8⁺ T cells in the injected tumors with either Myxoma-mCherry, MyxomaΔM064R, MVAΔE5R or PBS treatment. FIG. 173B shows the graph of absolute number of CD45⁺ cells in the injected tumors. Data are means±SEM (n=5-8). FIG. 173C shows the graph of percentage of Granzyme B⁺ CD8⁺ T cells out of CD8⁺ T cells. Data are means±SEM (n=5-8) (*P<0.05, t test).

FIG. 174A shows the representative dot plots of Granzyme B⁺ CD4⁺ T cells in the injected tumors with either Myxoma-mCherry, MyxomaΔM064R, MVAΔE5R or PBS treatment. FIG. 174B shows the graph of percentage of Granzyme B⁺ CD4⁺ T cells out of CD4⁺ T cells. Data are means±SEM (n=5-8) (**P<0.01; ***P<0.001; ****P<0.0001, t test).

### DETAILED DESCRIPTION

It is to be appreciated that certain aspects, modes, embodiments, variations, and features of the present technology are described below in various levels of detail in order to provide a substantial understanding of the present technology.

### I. Definitions

The definitions of certain terms as used in this specification are provided below. Unless defined otherwise, all technical and scientific terms used herein generally have the same meaning as commonly understood by one of ordinary skill in the art to which this present technology belongs.

As used in this specification and the appended claims, the singular forms “a,” “an,” and “the” include plural referents unless the content clearly dictates otherwise. For example, reference to “a cell” includes a combination of two or more cells, and the like.

As used herein, the term “about” encompasses the range of experimental error that may occur in a measurement and will be clear to the skilled artisan.

As used herein, the term “adjuvant” refers to a substance that enhances, augments, or potentiates the host's immune response to antigens, including tumor antigens.

As used herein, the “administration” of an agent or drug to a subject includes any route of introducing or delivering to a subject a compound to perform its intended function. Administration can be carried out by any suitable route, including but not limited to, orally, intranasally, parenterally (intravenously, intramuscularly, intradermally, intraperitoneally, or subcutaneously), rectally, intrathecally, intratumorally, or topically. Administration includes self-administration and the administration by another.

As used herein, the term “antigen” refers to a molecule to which an antibody (or antigen binding fragment thereof) can selectively bind. The target antigen may be a protein, carbohydrate, nucleic acid, lipid, hapten, or other naturally occurring or synthetic compound. In some embodiments, the antigen is contained within a whole cell, such as in a tumor antigen-containing whole cell vaccine. In some embodiments, the target antigen encompasses cancer-related antigens or neoantigens and includes proteins or other molecules expressed by tumor or non-tumor cancers, such as molecules that are present in cancer cells but absent in non-cancer cells, and molecules that are up-regulated in cancer cells as compared to non-cancer cells.

As used herein, “attenuated,” as used in conjunction with a virus, refers to a virus having reduced virulence or pathogenicity as compared to a non-attenuated counterpart, yet is still viable or live. Typically, attenuation renders an infectious agent, such as a virus, less harmful or virulent to an infected subject compared to a non-attenuated virus. This is in contrast to a killed or completely inactivated virus.

As used herein, “conjoint administration” refers to administration of a second therapeutic modality in combination with one or more engineered poxviruses of the present technology (e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, and/or MVAΔE5R-hFlt3L-OX40L-ΔWR199). For example, an immune checkpoint blocking agent, immunomodulatory agent, and/or anti-cancer drug administered in close temporal proximity with one or more engineered poxviruses of the present technology (e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, and/or MVAΔE5R-hFlt3L-OX40L-ΔWR199). For example, a PD-1/PD-L1 inhibitor and/or a CTLA-4 inhibitor (in more specific embodiments, an antibody) can be administered simultaneously (i.e., concurrently) with one or more engineered poxviruses of the present technology (e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, and/or MVAΔE5R-hFlt3L-OX40L-ΔWR199) (by intravenous or intratumoral injection when the MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, and/or MVAΔE5R-hFlt3L-OX40L-ΔWR199 is administered intratumorally or systemically as stated above) or before or after the MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, and/or MVAΔE5R-hFlt3L-OX40L-ΔWR199 administration. In some embodiments, if the MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, and/or MVAΔE5R-hFlt3L-OX40L-ΔWR199 administration and the immune checkpoint blocking agent, immunomodulatory agent, and/or anti-cancer drug are administered about 1 to about 7 days apart or even up to three weeks apart, this would still be within “close temporal proximity” as stated herein, therefore such administration will qualify as “conjoint.”

The term “corresponding wild-type strain” or “corresponding wild-type virus” is used herein to refer to the wild-type MVA, vaccinia virus (VACV), or myxoma virus (MYXV) strain from which the engineered MVA, vaccinia, or myxoma strain or virus was derived. As used herein, a wild-type MVA, vaccinia, or myxoma strain or virus is a strain or virus that has not been engineered to disrupt or delete (knock out) a particular gene of interest and/or to express a heterologous nucleic acid. For example, in some embodiments, a wild-type MVA, vaccinia, or myxoma strain or virus is a strain or virus that has not been engineered to disrupt or delete (knock out) the C7 gene and express OX40L. In other embodiments, a wild-type MVA, vaccinia, or myxoma strain or virus is a strain or virus that has not been engineered to disrupt or delete (knock out) the E5R (or M31R) gene. The engineered MVA, vaccinia, or myxoma strain or virus may have been modified to disrupt or delete (knock out) the C7 gene and express OX40L alone or in combination with further modifications (e.g., engineered to express additional immunomodulatory proteins and/or comprise additional gene deletions) as described herein. Additionally or alternatively, the engineered MVA, vaccinia, or myxoma strain or virus may have been modified to disrupt or delete (knock out) the E5R (or M31R) gene alone or in combination with further modifications (e.g., engineered to express additional immunomodulatory proteins and/or comprise additional gene deletions) as described herein. The term “corresponding MVAΔE3L strain” or “corresponding MVAΔE3L virus” is used herein to refer to the MVA strain or virus having an E3L deletion alone (i.e., an MVAΔE3L strain or virus comprising no other genetic deletions or additions). The term “corresponding MVAΔC7L strain” or “corresponding MVAΔC7L virus” is used herein to refer to the MVA strain or virus having a C7L deletion alone (i.e., an MVAΔC7L strain or virus comprising no other genetic deletions or additions). The term “corresponding MVAΔE5R strain” or “corresponding MVAΔE5R virus” is used herein to refer to the MVA strain or virus having an E5R deletion alone (i.e., an MVAΔE5R strain or virus comprising no other genetic deletions or additions). The term “corresponding VACVΔC7L strain” or “corresponding VACVΔC7L virus” is used herein to refer to the vaccinia strain or virus having a C7L deletion alone (i.e., a VACVΔC7L strain or virus comprising no other genetic deletions or additions). The term “corresponding VACVΔE5R strain” or “corresponding VACVΔE5R virus” is used herein to refer to the vaccinia strain or virus having an E5R deletion alone (i.e., a VACVΔE5R strain or virus comprising no other genetic deletions or additions).

As used herein, the terms “delivering” and “contacting” refer to depositing the one or more engineered poxviruses (e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, and/or MVAΔE5R-hFlt3L-OX40L-ΔWR199) of the present disclosure in the tumor microenvironment whether this is done by local administration to the tumor (intratumoral) or by, for example, intravenous route. The term focuses on engineered virus (e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, and/or MVAΔE5R-hFlt3L-OX40L-ΔWR199) that reaches the tumor itself. In some embodiments, “delivering” is synonymous with administering, but it is used with a particular administration locale in mind, e.g., intratumoral.

The terms “disruption” and “mutation” are used interchangeably herein to refer to a detectable and heritable change in the genetic material. Mutations may include insertions, deletions, substitutions (e.g., transitions, transversion), transpositions, inversions, knockouts, and combinations thereof. Mutations may involve only a single nucleotide (e.g., a point mutation or a single nucleotide polymorphism) or multiple nucleotides. In some embodiments, mutations are silent, that is, no phenotypic effect of the mutation is detected. In other embodiments, the mutation causes a phenotypic change, for example, the expression level of the encoded product is altered, or the encoded product itself is altered. In some embodiments, a disruption or mutation may result in a disrupted gene with decreased levels of expression of a gene product (e.g., protein or RNA) as compared to the wild-type strain. In other embodiments, a disruption or mutation may result in an expressed protein with activity that is lower as compared to the activity of the expressed protein from the wild-type strain.

As used herein, an “effective amount” or “therapeutically effective amount” refers to a sufficient amount of an agent, which, when administered at one or more dosages and for a period of time, is sufficient to provide a desired biological result in alleviating, curing, or palliating a disease. In the present disclosure, an effective amount of one or more engineered poxviruses of the present technology (e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, and/or MVAΔE5R-hFlt3L-OX40L-ΔWR199) comprises an amount that (when administered for a suitable period of time and at a suitable frequency) reduces the number of cancer cells; or reduces the tumor size or eradicates the tumor; or inhibits (i.e., slows down or stops) cancer cell infiltration into peripheral organs; inhibits (i.e., slows down or stops) metastatic growth; inhibits (stabilizes or arrests) tumor growth; allows for treatment of the tumor; and/or induces and promotes an immune response against the tumor. An appropriate therapeutic amount in any individual case may be determined by one of ordinary skill in the art using routine experimentation in light of the present disclosure. Such determination may begin with amounts found effective in vitro and amounts found effective in animals. The therapeutically effective amount will be initially determined based on the concentration or concentrations found to confer a benefit to cells in culture. Effective amounts can be extrapolated from data within the cell culture and can be adjusted up or down based on factors such as detailed herein. Effective amounts of the viral constructs are generally within the range of about 10⁵ to about 10¹⁰ plaque forming units (pfu), although a lower or higher dose may be administered. In some embodiments, the dosage is about 10⁶-10⁹ pfu. In some embodiments, a unit dosage is administered in a volume within the range from 1 to 10 mL. The equivalence of pfu to virus particles can differ according to the specific pfu titration method used. Generally, pfu is equal to about 5 to 100 virus particles. A therapeutically effective amount the hFlt3L transgene bearing viruses can be administered in one or more divided doses for a prescribed period of time and at a prescribed frequency of administration. For example, a therapeutically effective amount of hFlt3L bearing viruses in accordance with the present disclosure may vary according to factors such as the disease state, age, sex, weight, and general condition of the subject, and the potency of the viral constructs to elicit a desired immunological response in the particular subject for the particular cancer.

With particular reference to the viral-based immunostimulatory agents disclosed herein, an “effective amount” or “therapeutically effective amount” refers to an amount of a composition comprising one or more one or more engineered poxviruses of the present technology (e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, and/or MVAΔE5R-hFlt3L-OX40L-ΔWR199) sufficient to reduce, inhibit, or abrogate tumor cell growth, thereby reducing or eradicating the tumor, or sufficient to inhibit, reduce or abrogate metastatic spread either in vitro, ex vivo, or in a subject or to elicit and promote an immune response against the tumor that will eventually result in one or more of metastatic spread reduction, inhibition, and/or abrogation as the case may be. The reduction, inhibition, or eradication of tumor cell growth may be the result of necrosis, apoptosis, or an immune response, or a combination of two or more of the foregoing (however, the precipitation of apoptosis, for example, may not be due to the same factors as observed with oncolytic viruses). The amount that is therapeutically effective may vary depending on such factors as the particular virus used in the composition, the age and condition of the subject being treated, the extent of tumor formation, the presence or absence of other therapeutic modalities, and the like. Similarly, the dosage of the composition to be administered and the frequency of its administration will depend on a variety of factors, such as the potency of the active ingredient, the duration of its activity once administered, the route of administration, the size, age, sex, and physical condition of the subject, the risk of adverse reactions and the judgment of the medical practitioner. The compositions are administered in a variety of dosage forms, such as injectable solutions.

With particular reference to combination therapy with an immune checkpoint inhibitor, an “effective amount” or “therapeutically effective amount” for an immune checkpoint blocking agent means an amount of an immune checkpoint blocking agent sufficient to reverse or reduce immune suppression in the tumor microenvironment and to activate or enhance host immunity in the subject being treated. Immune checkpoint blocking agents include, but are not limited to, inhibitory antibodies against CD28 inhibitor such as CTLA-4 (cytotoxic T lymphocyte antigen 4) (e.g., ipilimumab), anti-PD-1 (programmed Death 1) inhibitory antibodies (e.g., nivolumab, pembrolizumab, pidilizumab, lambrolizumab), and anti-PD-L1 (Programmed death ligand 1) inhibitory antibodies (MPDL3280A, BMS-936559, MEDI4736, MSB 00107180), as well as inhibitory antibodies against LAG-3 (lymphocyte activation gene 3), TIM3 (T-cell immunoglobulin and mucin-3), B7-H3, TIGIT (T-cell immunoreceptor with Ig and ITIM domains), AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, or PDR001, and combinations thereof. Dosage ranges of the foregoing are known or readily within the skill in the art as several dosing clinical trials have been completed, making extrapolation to other agents possible.

In some embodiments, the tumor expresses the particular checkpoint, but in the context of the present technology, this is not strictly necessary as immune checkpoint blocking agents block more generally immune suppressive mechanisms within the tumors, elicited by tumor cells, stromal cells, and tumor-infiltrating immune cells.

For example, the CTLA-4 inhibitor ipilimumab, when administered as adjuvant therapy after surgery in melanoma, is administered at 1-2 mg/mL over 90 minutes for a total infusion amount of 3 mg/kg every three weeks for a total of 4 doses. This therapy is often accompanied by severe, even life-threatening, immune-mediated adverse reactions, which limits the tolerated dose as well as the cumulative amount that can be administered. It is anticipated that it will be possible to reduce the dose and/or cumulative amount of ipilimumab when it is administered conjointly with one or more engineered poxviruses of the present technology (e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, and/or MVAΔE5R-hFlt3L-OX40L-ΔWR199). In particular, in light of the experimental results set forth below, it is anticipated that it will be further possible to reduce the CTLA-4 inhibitor's dose if it is administered directly to the tumor conjointly with one or both the foregoing MVA viruses. Accordingly, the amounts provided above for ipilimumab may be a starting point for determining the particular dosage and cumulative amount to be given to a patient in conjoint administration.

As another example, pembrolizumab is prescribed for administration as adjuvant therapy in melanoma diluted to 25 mg/mL. It is administered at a dosage of 2 mg/kg over 30 minutes every three weeks. This may be a starting point for determining dosage and administration in the conjoint administration of one or more engineered poxviruses of the present technology (e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, and/or MVAΔE5R-hFlt3L-OX40L-ΔWR199).

Nivolumab could also serve as a starting point in determining the dosage and administration regimen of checkpoint inhibitors administered in combination with one or more engineered poxviruses of the present technology (e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, and/or MVAΔE5R-hFlt3L-OX40L-ΔWR199). Nivolumab is prescribed for administration at 3 mg/kg as an intravenous infusion over 60 minutes every two weeks.

Immune stimulating agents such as agonist antibodies have also been explored as immunotherapy for cancers. For example, anti-ICOS antibody binds to the extracellular domain of ICOS leading to the activation of ICOS signaling and T-cell activation. Anti-OX40 antibody can bind to OX40 and potentiate T-cell receptor signaling leading to T-cell activation, proliferation and survival. Other examples include agonist antibodies against 4-1BB (CD137), GITR.

The immune stimulating agonist antibodies can be used systemically in combination with intratumoral injection of one or more engineered poxviruses of the present technology (e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, and/or MVAΔE5R-hFlt3L-OX40L-ΔWR199). Alternatively, the immune stimulating agonist antibodies can be used conjointly with one or more engineered poxviruses of the present technology (e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, and/or MVAΔE5R-hFlt3L-OX40L-ΔWR199) via intratumoral delivery either simultaneously (i.e., concurrently) or sequentially.

The term “immunomodulatory drug” is used herein to refer to Fingolimod (FTY720).

The terms “engineered” or “genetically engineered” are used herein to refer to an organism that has been manipulated to be genetically altered, modified, or changed, e.g., by disruption of the genome. For example, an “engineered vaccinia virus strain,” “engineered modified vaccinia Ankara virus,” or “engineered myxoma virus” refers to a vaccinia, modified vaccinia Ankara, or myxoma strain that has been manipulated to be genetically altered, modified, or changed. In the present context, “engineered” or “genetically engineered” includes recombinant vaccinia viruses, recombinant modified vaccinia Ankara viruses, and recombinant myxoma viruses.

The term “gene cassette” is used herein to refer to a DNA sequence encoding and capable of expressing one or more genes of interest (e.g., OX40L, hFlt3L, a selectable marker, or a combination thereof) that can be inserted between one or more selected restriction sites of a DNA sequence. In some embodiments, insertion of a gene cassette results in a disrupted gene. In some embodiments, disruption of the gene involves replacement of at least a portion of the gene with a gene cassette, which includes a nucleotide sequence encoding a gene of interest (e.g., OX40L, hFlt3L, a selectable marker, or a combination thereof).

As used herein, “heterologous nucleic acid,” refers to a nucleic acid, DNA, or RNA, which has been introduced into a virus, and which is not a copy of a sequence naturally found in the virus into which it is introduced. Such heterologous nucleic acid may comprise segments that are a copy of a sequence that is naturally found in the virus into which it has been introduced.

As used herein, wherever a gene is described, the gene may be either human or murine such that the designation of human (h or hu) or murine (m or mu) may be used interchangeably and is not intended to be limiting. For example, where mIL-12 is described, hIL-12 may be substituted for mIL-12 in the described constructs, and vice versa.

As used herein, “IL-15/IL-15Rα” encompasses membrane bound hIL-15/IL-15Rα transpresentation constructs and fusion proteins as described in Van den Bergh et al. (Pharmacology & Therapeutics 170:73-79 (2017); Kowalsky et al. (Molecular Therapy 26(10):2476-2486 (2018); Stoklasek et al. (J. Immunol. 177:6072-6080); Duboi et al. (J. Immunol. 180:2099-2106 (2008); Epardaud et al, (Cancer Res. 68:2972-2983 (2008); and Dubois et al. (Immunity 17:537-547 (2002), each of which is herein incorporated by reference.

As used herein, “immune checkpoint inhibitor” or “immune checkpoint blocking agent” or “immune checkpoint blockade inhibitor” refers to molecules that completely or partially reduce, inhibit, interfere with, or modulate the activity of one or more checkpoint proteins. Checkpoint proteins regulate T-cell activation or function. Checkpoint proteins include, but are not limited to, CD28 receptor family members, CTLA-4 and its ligands CD80 and CD86; PD-1 and its ligands PD-L1 and PD-L2; LAG3, B7-H3, B7-H4, TIM3, ICOS, II DLBCL, BTLA or any combination of two or more of the foregoing. Non-limiting examples of immune checkpoint blocking agents contemplated for use herein include, but are not limited to, inhibitory antibodies against CD28 inhibitor such as CTLA-4 (cytotoxic T lymphocyte antigen 4) (e.g., ipilimumab), anti-PD-1 (programmed Death 1) inhibitory antibodies (e.g., nivolumab, pembrolizumab, pidilizumab, lambrolizumab), and anti-PD-L1 (Programmed death ligand 1) inhibitory antibodies (MPDL3280A, BMS-936559, MEDI4736, MSB 00107180), as well as inhibitory antibodies against LAG-3 (lymphocyte activation gene 3), TIM3 (T-cell immunoglobulin and mucin-3), B7-H3, TIGIT (T-cell immunoreceptor with Ig and ITIM domains), AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, or BTLA, PDR001, and combinations thereof.

As used herein, “immune response” refers to the action of one or more of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells, metastatic tumor cells, etc. An immune response may include a cellular response, such as a T-cell response that is an alteration (modulation, e.g., significant enhancement, stimulation, activation, impairment, or inhibition) of cellular, i.e., T-cell function. A T-cell response may include generation, proliferation or expansion, or stimulation of a particular type of T-cell, or subset of T-cells, for example, effector CD4⁺, CD4⁺ helper, effector CD8⁺, CD8⁺cytotoxic, or natural killer (NK) cells. Such T-cell subsets may be identified by detecting one or more cell receptors or cell surface molecules (e.g., CD or cluster of differentiation molecules). A T-cell response may also include altered expression (statistically significant increase or decrease) of a cellular factor, such as a soluble mediator (e.g., a cytokine, lymphokine, cytokine binding protein, or interleukin) that influences the differentiation or proliferation of other cells. For example, Type I interferon (IFN-α/β) is a critical regulator of the innate immunity (Huber et al., Immunology 132(4):466-474 (2011)). Animal and human studies have shown a role for IFN-α/β in directly influencing the fate of both CD4⁺ and CD8⁺ T-cells during the initial phases of antigen recognition and anti-tumor immune response. IFN Type I is induced in response to activation of dendritic cells, in turn a sentinel of the innate immune system. An immune response may also include humoral (antibody) response.

The term “immunogenic composition” is used herein to refer to a composition that will elicit an immune response in a mammal that has been exposed to the composition. In some embodiments, an immunogenic composition comprises MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, and/or MVAΔE5R-hFlt3L-OX40L-ΔWR199, an antigen, an adjuvant comprising any one or more of the foregoing engineered viruses, and/or an adjuvant comprising MVAΔC7L-hFlt3L-TK(−)-OX40L, MVAΔE5R-hFlt3L-OX40L, MVAΔC7LΔE5R-hFlt3L-OX40L, and/or Heat-iMVAΔE5R alone or in combination with immune checkpoint blockade inhibitors. As used herein, an immunogenic composition encompasses vaccines. In some embodiments, the immunogenic composition comprises a tumor antigen-containing whole cell vaccine (e.g., an irradiated whole cell vaccine).

As used herein, the term “inactivated MVA” refers to heat-inactivated MVA (Heat-iMVA) and/or UV-inactivated MVA which are infective, nonreplicative, and do not suppress IFN Type I production in infected DC cells. As used herein, the term “inactivated vaccinia virus” includes heat-inactivated vaccinia virus and/or UV-inactivated vaccinia virus. MVA or vaccinia virus inactivated by a combination of heat and UV radiation is also within the scope of the present disclosure.

As used herein, “Heat-inactivated MVA” (Heat-iMVA) and “Heat-inactivated vaccinia virus” refer to MVA and vaccinia virus, respectively, which have been exposed to heat treatment under conditions that do not destroy its immunogenicity or its ability to enter target cells (tumor cells) but remove residual replication ability of the virus as well as factors that inhibit the host's immune response. An example of such conditions is exposure to a temperature within the range of about 50 to about 60° C. for a period of time of about an hour. Other times and temperatures can be determined by one of skill in the art.

As used herein, “UV-inactivated MVA” and “UV-inactivated vaccinia virus” refer to MVA and vaccinia virus, respectively, that have been inactivated by exposure to UV under conditions that do not destroy its immunogenicity or its ability to enter target cells (tumor cells) but remove residual replication ability of the virus. An example of such conditions, which can be useful in the present methods, is exposure to UV using, for example, a 365 nm UV bulb for a period of about 30 min to about 1 hour. Other limits of these conditions of UV wavelength and exposure can be determined by one of skill in the art.

A “knock out,” “knocked out gene,” or a “gene deletion” refers to a gene including a null mutation (e.g., the wild-type product encoded by the gene is not expressed, expressed at levels so low as to have no effect, or is non-functional). In some embodiments, the knocked out gene includes heterologous sequences (e.g., one or more gene cassettes comprising a heterologous nucleic acid sequence) or genetically engineered non-functional sequences of the gene itself, which renders the gene non-functional. In other embodiments, the knocked out gene is lacking a portion of the wild-type gene. For example, in some embodiments, at least about 10%, at least about 20%, at least about 30%, at least about 40%, or at least about 60% of the wild-type gene sequence is deleted. In other embodiments, the knocked out gene is lacking at least about 70%, at least about 75%, at least about 80%, at least about 90%, at least about 95% or at least about 100% of the wild-type gene sequence. In other embodiments, the knocked out gene may include up to 100% of the wild-type gene sequence (e.g., some portion of the wild-type gene sequence may be deleted) but also include one or more heterologous and/or non-functional nucleic acid sequences inserted therein.

As used herein, “metastasis” refers to the spread of cancer from its primary site to neighboring tissues or distal locations in the body. Cancer cells (including cancer stem cells) can break away from a primary tumor, penetrate lymphatic and blood vessels, circulate through the bloodstream, and grow in normal tissues elsewhere in the body. Metastasis is a sequential process, contingent on tumor cells (or cancer stem cells) breaking off from the primary tumor, traveling through the bloodstream or lymphatics, and stopping at a distant site. Once at another site, cancer cells re-penetrate through the blood vessels or lymphatic walls, continue to multiply, and eventually form a new tumor (metastatic tumor). In some embodiments, this new tumor is referred to as a metastatic (or secondary) tumor.

As used herein, “MVA” means “modified vaccinia Ankara” and refers to a highly attenuated strain of vaccinia derived from the Ankara strain and developed for use as a vaccine and vaccine adjuvant. The original MVA was isolated from the wild-type Ankara strain by successive passage through chicken embryonic cells. Treated thus, it lost about 15% of the genome of wild-type vaccinia including its ability to replicate efficiently in primate (including human) cells. (Mayr et al., Zentralbl Bakteriol B 167:375-390 (1978)). MVA is considered an appropriate candidate for development as a recombinant vector for gene or vaccination delivery against infectious diseases or tumors. (Verheust et al., Vaccine 30(16):2623-2632 (2012)). MVA has a genome of 178 kb in length and a sequence first disclosed in Antoine et al., Virol. 244(2): 365-396 (1998). Sequences are also disclosed in GenBank Accession No. U94848.1 (SEQ ID NO: 1). Clinical grade MVA is commercially and publicly available from Bavarian Nordic A/S Kvistgaard, Denmark. Additionally, MVA is available from ATCC, Rockville, MD, and from CMCN (Institut Pasteur Collection Nationale des Microorganismes) Paris, France.

The term “MVAΔC7L,” is used herein to refer to a modified vaccinia Ankara (MVA) mutant virus or a vaccine comprising the virus, in which the C7 gene is not expressed, expressed at levels so low as to have no effect, or the expressed protein is non-functional (e.g., is a null-mutation). As used herein, “MVAΔC7L” includes a deletion mutant of MVA which lacks a functional C7L gene and is infective but non-replicative and it is further impaired in its ability to evade the host's immune system. As used herein, “MVAΔC7L” encompasses a recombinant MVA virus that does not express a functional C7 protein. In some embodiments, the ΔC7L mutant includes a heterologous nucleic acid sequence in place of all or a majority of the C7L gene sequence. For example, as used herein, “MVAΔC7L” encompasses a recombinant MVA nucleic acid sequence, wherein the nucleic acid sequence corresponding to the position of C7 in the MVA genome (e.g., position 18,407 to 18,859 of SEQ ID NO: 1) is replaced with a heterologous nucleic acid sequence comprising an open reading frame that encodes a specific gene of interest (SG), such as human OX40L (“MVAΔC7L-OX40L”) or human Fms-like tyrosine kinase 3 ligand (hFlt3L) (“MVAΔC7L-hFlt3L”). In some embodiments, the heterologous nucleic acid sequence comprises an open reading frame that encodes a selectable marker. In some embodiments, the selectable marker is a fluorescent protein (e.g., gpt, GFP, mCherry). In some embodiments, the MVAΔC7L virus encompasses a recombinant MVA virus that does not express a functional thymidine kinase (TK) protein. In some embodiments, a specific gene of interest (e.g., OX40L, hFlt3L) is inserted into the TK locus of the MVA genome (e.g., position 75,560 to 76,093 of SEQ ID NO: 1), splitting the TK gene and obliterating it (“MVAΔC7L-OX40L-TK(−)”; “MVAΔC7L-hFlt3L-TK(−)”). In some embodiments, MVAΔC7L encompasses a recombinant MVA virus in which all or a majority of the C7L gene sequence is replaced by a first specific gene of interest (e.g., hFlt3L) and a second gene of interest (e.g., OX40L) is inserted into the TK locus (“MVAΔC7L-hFlt3L-TK(−)-OX40L”). In some embodiments, the recombinant MVAΔC7L-OX40L viruses of the present technology are further modified to express at least one further heterologous gene, such as any one or more of hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L (where “h” or “hu” designates the human protein), and/or include at least one further viral gene mutation or deletion, such as any one or more of the following deletions: E3L (ΔE3L); E3LΔ83N; B2R (ΔB2R), B19R (B18R; ΔWR200); E5R; K7R; C12L (IL18BP); B8R; B14R; N1L; C11R; K1L; M1L; N2L; and/or WR199. For example, in some embodiments, MVAΔC7L-hFlt3L-TK(−)-OX40 is further modified to comprise a deletion of E5R, in which the E5R gene is replaced by a selectable marker (e.g., mCherry) through homologous recombination at the E4L and E6R loci. In some embodiments, MVAΔC7L is modified to express one or more heterologous genes from within other loci, such as the E5R locus. For example, in some embodiments, MVAΔC7L encompasses a recombinant MVA virus in which all or a majority of the E5R gene sequence is replaced by a first specific gene of interest (e.g., hFtl3L) and a second specific gene of interest (e.g., OX40L), wherein the coding sequences of the first and second specific genes of interest are separated by a cassette including a furin cleavage site followed by a 2A peptide (Pep2A) sequence, thereby forming a recombinant virus such as “MVAΔC7LΔE5R-hFlt3L-OX40L.” In other embodiments, the recombinant MVAΔC7L-OX40L viruses of the present technology contain no further heterologous genes and/or viral gene mutations other than those specifically referred to in the name of the virus.

The term “MVAΔE3L” means a deletion mutant of MVA which lacks a functional E3L gene and is infective but non-replicative and it is further impaired in its ability to evade the host's immune system. It has been used as a vaccine vector to transfer tumor or viral antigens. The mutant MVA E3L knockout and its preparation have been described in U.S. Pat. No. 7,049,145, for example. As used herein, “MVAΔE3L” encompasses a recombinant MVA modified to express a specific gene of interest (SG), such as OX40L (“MVAΔE3L-OX40L”). In some embodiments, the MVAΔE3L virus encompasses a recombinant MVA virus that does not express a functional thymidine kinase (TK) protein. In some embodiments, a specific gene of interest (e.g., OX40L, hFlt3L) is inserted into the TK locus, splitting the TK gene and obliterating it (“MVAΔE3L-OX40L-TK(−)”; “MVAΔE3L-hFlt3L-TK(−)”). In some embodiments, the recombinant MVAΔE3L-OX40L viruses of the present technology are further modified to express at least one further heterologous gene, such as any one or more hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or include at least one viral gene mutation or deletion, such as any one or more of the following deletions: B2R (ΔB2R), B19R (B18R; ΔWR200); E5R; K7R; C12L (IL18BP); B8R; B14R; N1L; C11R; K1L; M1L; N2L; and/or WR199. In other embodiments, the recombinant MVAΔE3L-OX40L viruses of the present technology do not express any further heterologous genes and/or do not include any additional viral gene mutations or deletions other than those specifically indicated in the name of the virus.

The term “MVAΔE5R,” is used herein to refer to a modified vaccinia Ankara (MVA) mutant virus or a vaccine comprising the virus, in which the E5R gene is not expressed, expressed at levels so low as to have no effect, or the expressed protein is non-functional (e.g., is a null-mutation). As used herein, “MVAΔE5R” includes a deletion mutant of MVA which lacks a functional E5R gene and is infective but non-replicative and it is further impaired in its ability to evade the host's immune system. As used herein, “MVAΔE5R” encompasses a recombinant MVA virus that does not express a functional E5 protein. In some embodiments, the ΔE5R mutant includes a heterologous nucleic acid sequence in place of all or a majority of the E5R gene sequence. For example, as used herein, “MVAΔE5R” encompasses a recombinant MVA nucleic acid sequence, wherein the nucleic acid sequence corresponding to the position of E5R in the MVA genome (e.g., position 38,432 to 39,385 of SEQ ID NO: 1) is replaced with a heterologous nucleic acid sequence comprising an open reading frame that encodes a specific gene of interest (SG), such as human OX40L (“MVAΔE5R-OX40L”) or human Fms-like tyrosine kinase 3 ligand (hFlt3L) (“MVAΔE5R-hFlt3L”). In some embodiments, MVAΔE5R encompasses a recombinant MVA wherein the E5R locus is modified to express one or more heterologous genes. For example, in some embodiments, MVAΔE5R encompasses a recombinant MVA in which all or a majority of the E5R gene sequence is replaced by a first specific gene of interest (e.g., hFtl3L) and a second specific gene of interest (e.g., OX40L), wherein the coding sequences of the first and second specific genes of interest are separated by a cassette including a furin cleavage site followed by a 2A peptide (Pep2A) sequence, thereby forming a recombinant virus such as “MVAΔE5R-hFlt3L-OX40L.” In some embodiments, the heterologous nucleic acid sequence comprises an open reading frame that encodes a selectable marker. In some embodiments, the selectable marker is a fluorescent protein (e.g., gpt, GFP, mCherry). In some embodiments, the MVAΔE5R virus encompasses a recombinant MVA virus that does not express a functional thymidine kinase (TK) protein. In some embodiments, a specific gene of interest (e.g., OX40L, hFlt3L) is inserted into the TK locus of the MVA genome (e.g., position 75,560 to 76,093 of SEQ ID NO: 1), splitting the TK gene and obliterating it (“MVAΔE5R-OX40L-TK(−)”; “MVAΔE5R-hFlt3L-TK(−)”). In some embodiments, MVAΔE5R encompasses a recombinant MVA virus in which all or a majority of the E5R gene sequence is replaced by a first specific gene of interest (e.g., hFlt3L) and a second gene of interest (e.g., OX40L) is inserted into the TK locus (“MVAΔE5R-hFlt3L-TK(−)-OX40L”). In some embodiments, the engineered MVAΔE5R viruses of the present technology are modified to express at least one heterologous gene, such as any one or more of hOX40L, hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L (where “h” or “hu” designates the human protein), and/or include at least one further viral gene mutation or deletion, such as any one or more of the following deletions: E3L (ΔE3L); E3LΔ83N; C7L (ΔC7L); B2R (ΔB2R), B19R (B18R; ΔWR200); E5R; K7R; C12L (IL18BP); B8R; B14R; N1L; C11R; K1L; M1L; N2L; and/or WR199. In other embodiments, the MVAΔE5R viruses of the present technology contain no further heterologous genes and/or viral gene mutations other than those specifically referred to in the name of the virus.

The term “MVAΔWR199,” is used herein to refer to a modified vaccinia Ankara (MVA) mutant virus or a vaccine comprising the virus, in which the WR199 gene is not expressed, expressed at levels so low as to have no effect, or the expressed protein is non-functional (e.g., is a null-mutation). As used herein, “MVAΔWR199” includes a deletion mutant of MVA which lacks a functional WR199 gene and is infective but non-replicative and it is further impaired in its ability to evade the host's immune system. As used herein, “MVAΔWR199” encompasses a recombinant MVA virus that does not express a functional E5 protein. In some embodiments, the ΔWR199 mutant includes a heterologous nucleic acid sequence in place of all or a majority of the WR199 gene sequence. For example, as used herein, “MVAΔWR199” encompasses a recombinant MVA nucleic acid sequence, wherein the nucleic acid sequence corresponding to the position of WR199 in the MVA genome (e.g., position 158,399 to 160,143 of the sequence set forth in GenBank Accession No. AY603355) is replaced with a heterologous nucleic acid sequence comprising an open reading frame that encodes a specific gene of interest (SG), such as human OX40L (“MVAΔWR199-OX40L”) or human Fms-like tyrosine kinase 3 ligand (hFlt3L) (“MVAΔWR199-hFlt3L”). In some embodiments, MVAΔWR199 encompasses a recombinant MVA wherein the WR199 locus is modified to express one or more heterologous genes. For example, in some embodiments, MVAΔWR199 encompasses a recombinant MVA in which all or a majority of the WR199 gene sequence is replaced by a first specific gene of interest (e.g., hFtl3L) and a second specific gene of interest (e.g., OX40L), wherein the coding sequences of the first and second specific genes of interest are separated by a cassette including a furin cleavage site followed by a 2A peptide (Pep2A) sequence, thereby forming a recombinant virus such as “MVAΔWR199-hFlt3L-OX40L.” In some embodiments, the heterologous nucleic acid sequence comprises an open reading frame that encodes a selectable marker. In some embodiments, the selectable marker is a fluorescent protein (e.g., gpt, GFP, mCherry). In some embodiments, the MVAΔWR199 virus encompasses a recombinant MVA virus that does not express a functional thymidine kinase (TK) protein. In some embodiments, a specific gene of interest (e.g., OX40L, hFlt3L) is inserted into the TK locus of the MVA genome (e.g., position 75,560 to 76,093 of SEQ ID NO: 1), splitting the TK gene and obliterating it (“MVAΔWR199-OX40L-TK(−)”; “MVAΔWR199-hFlt3L-TK(−)”). In some embodiments, MVAΔWR199 encompasses a recombinant MVA virus in which all or a majority of the WR199 gene sequence is replaced by a first specific gene of interest (e.g., hFlt3L) and a second gene of interest (e.g., OX40L) is inserted into the TK locus (“MVAΔWR199-hFlt3L-TK(−)-OX40L”). In some embodiments, the engineered MVAΔWR199 viruses of the present technology are modified to express at least one heterologous gene, such as any one or more of hOX40L, hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L (where “h” or “hu” designates the human protein), and/or include at least one further viral gene mutation or deletion, such as any one or more of the following deletions: E3L (ΔE3L); E3LΔ83N; C7L (ΔC7L); B2R (ΔB2R), B19R (B18R; ΔWR200); E5R; K7R; C12L (IL18BP); B8R; B14R; N1L; C11R; K1L; M1L; and/or N2L. In other embodiments, the MVAΔWR199 viruses of the present technology contain no further heterologous genes and/or viral gene mutations other than those specifically referred to in the name of the virus.

The term “VACVΔC7L,” is used herein to refer to a vaccinia mutant virus or vaccine comprising the virus in which the C7 gene is not expressed, expressed at levels so low as to have no effect, or the expressed protein is non-functional (e.g., is a null-mutation). As used herein, “VACVΔC7L” encompasses a recombinant vaccinia virus (VACV) that does not express a functional C7 protein. In some embodiments, the vaccinia virus is derived from the Western Reserve (WR) strain. In some embodiments, the ΔC7L mutant includes a heterologous sequence in place of all or a majority of the C7L gene sequence. For example, as used herein, “VACVΔC7L” encompasses a recombinant vaccinia virus nucleic acid sequence, wherein the nucleic acid sequence corresponding to the position of C7 in the VACV genome (e.g., position 15,716 to 16,168 of SEQ ID NO: 2) is replaced with a heterologous nucleic acid sequence comprising an open reading frame that encodes a specific gene of interest (SG), such as human OX40L (“VACVΔC7L-OX40L”) or human Fms-like tyrosine kinase 3 ligand (hFlt3L) gene (“VACVΔC7L-hFlt3L”). In some embodiments, the heterologous nucleic acid sequence comprises an open reading frame that encodes a selectable marker. In some embodiments, the selectable marker is a fluorescent protein (e.g., gpt, GFP, mCherry). In some embodiments, the VACVΔC7L virus encompasses a recombinant vaccinia virus that does not express a functional thymidine kinase (TK) protein. In some embodiments, a specific gene of interest (e.g., OX40L, hFlt3L) is inserted into the TK locus (e.g., position 80,962 to 81,032 of SEQ ID NO: 2), splitting the TK gene and obliterating it (“VACVΔC7L-OX40L-TK(−)”; “VACVΔC7L-hFlt3L-TK(−)”). In some embodiments, VACVΔC7L encompasses a recombinant vaccinia virus in which all or a majority of the C7L gene sequence is replaced by a first specific gene of interest (e.g., hFlt3L) and a second gene of interest (e.g., OX40L) is inserted into the TK locus (“VACVΔC7L-hFlt3L-TK(−)-OX40L”). In some embodiments, the recombinant VACVΔC7L-OX40L viruses of the present technology are further modified to express at least one additional heterologous gene, such as any one or more of hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or include at least one viral gene mutation or deletion, such as any one or more of the following vaccinia viral deletions: E3L (ΔE3L); E3LΔ83N; B2R (ΔB2R), B19R (B18R; ΔWR200); E5R; K7R; C12L (IL18BP); B8R; B14R; N1L; C11R; K1L; M1L; N2L; and/or WR199. For example, in some embodiments, the disclosure of the present technology provides a recombinant VACVΔ E3L83N-hFlt3L-anti-CTLA-4-ΔC7L-OX40L virus. In other embodiments, the recombinant VACVΔC7L-OX40L viruses of the present technology do not express any further heterologous genes and/or do not include any additional viral gene mutations or deletions other than those specifically indicated in the name of the virus.

The term “VACVΔE5R,” is used herein to refer to a vaccinia mutant virus or vaccine comprising the virus in which the E5R gene is not expressed, expressed at levels so low as to have no effect, or the expressed protein is non-functional (e.g., is a null-mutation). As used herein, “VACVΔE5R” encompasses a recombinant vaccinia virus (VACV) that does not express a functional E5 protein. In some embodiments, the vaccinia virus is derived from the Western Reserve (WR) strain. In some embodiments, the ΔE5R mutant includes a heterologous sequence in place of all or a majority of the E5R gene sequence. For example, as used herein, “VACVΔE5R” encompasses a recombinant vaccinia virus nucleic acid sequence, wherein the nucleic acid sequence corresponding to the position of E5R in the VACV genome (e.g., position 49,236 to 50,261 of SEQ ID NO: 2) is replaced with a heterologous nucleic acid sequence comprising an open reading frame that encodes a specific gene of interest (SG), such as human OX40L (“VACVΔE5R-OX40L”) or human Fms-like tyrosine kinase 3 ligand (hFlt3L) gene (“VACVΔE5R-hFlt3L”). In some embodiments, the heterologous nucleic acid sequence comprises an open reading frame that encodes a selectable marker. In some embodiments, the selectable marker is a fluorescent protein (e.g., gpt, GFP, mCherry). In some embodiments, the VACVΔE5R virus encompasses a recombinant vaccinia virus that does not express a functional thymidine kinase (TK) protein. In some embodiments, a specific gene of interest (e.g., OX40L, hFlt3L) is inserted into the TK locus (e.g., position 80,962 to 81,032 of SEQ ID NO: 2), splitting the TK gene and obliterating it (“VACVΔE5R-OX40L-TK(−)”; “VACVΔE5R-hFlt3L-TK(−)”). In some embodiments, VACVΔE5R encompasses a recombinant vaccinia virus in which all or a majority of the E5R gene sequence is replaced by a first specific gene of interest (e.g., hFlt3L) and a second gene of interest (e.g., OX40L) is inserted into the TK locus (“VACVΔE5R-hFlt3L-TK(−)-OX40L”). In some embodiments, the engineered VACVΔE5R viruses of the present technology are modified to express at least one heterologous gene, such as any one or more of hOX40L, hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or include at least one viral gene mutation or deletion, such as any one or more of the following vaccinia viral deletions: E3L (ΔE3L); E3LΔ83N; C7L (ΔC7L); B2R (ΔB2R), B19R (B18R; ΔWR200); E5R; K7R; C12L (IL18BP); B8R; B14R; N1L; C11R; K1L; M1L; N2L; and/or WR199. For example, in some embodiments, the disclosure of the present technology provides a recombinant VACVΔE3L-hFlt3L-anti-CTLA-4-OX40L-ΔE5R virus. As another example, in some embodiments, the TK locus of the vaccinia genome is modified through homologous recombination to express both the heavy and light chain of an antibody, such as anti-CTLA-4, wherein the coding sequences of the heavy chain and light chain are separated by a cassette including a furin cleavage site followed by a 2A peptide (Pep2A) sequence to produce VACV-TK(−)-anti-CTLA-4. In some embodiments, the VACV-TK(−)-anti-CTLA-4 genome is further modified to comprise a deletion of E5R, in which all or a majority of the E5R gene sequence is replaced by a first specific gene of interest (e.g., hFlt3L) and a second specific gene of interest (e.g., OX40L), wherein the coding sequences of the first and second specific genes of interest are separated by a cassette including a furin cleavage site followed by a 2A peptide (Pep2A) sequence, thereby forming a recombinant virus such as VACV-TK⁻-anti-CTLA-4-E5R⁻-hFlt3L-OX40L (or VACVΔE5R-TK(−)-anti-CTLA-4-hFlt3L-OX40L). In other embodiments, the VACVΔE5R viruses of the present technology do not express any further heterologous genes and/or do not include any additional viral gene mutations or deletions other than those specifically indicated in the name of the virus.

The term “VACVΔB2R,” is used herein to refer to a vaccinia mutant virus or vaccine comprising the virus in which the B2R gene is not expressed, expressed at levels so low as to have no effect, or the expressed protein is non-functional (e.g., is a null-mutation). As used herein, “VACVΔB2R” encompasses a recombinant vaccinia virus (VACV) that does not express a functional B2 protein. In some embodiments, the vaccinia virus is derived from the Western Reserve (WR) strain. In some embodiments, the ΔB2R mutant includes a heterologous sequence in place of all or a majority of the B2R gene sequence. For example, as used herein, “VACVΔB2R” encompasses a recombinant vaccinia virus nucleic acid sequence, wherein the nucleic acid sequence corresponding to the position of B2R in the VACV genome (e.g., position 164,856 to 165,530 of SEQ ID NO: 2) is replaced with a heterologous nucleic acid sequence comprising an open reading frame that encodes a specific gene of interest (SG), such as human OX40L (“VACVΔB2R-OX40L”) or human Fms-like tyrosine kinase 3 ligand (hFlt3L) gene (“VACVΔB2R-hFlt3L”). In some embodiments, the heterologous nucleic acid sequence comprises an open reading frame that encodes a selectable marker. In some embodiments, the selectable marker is a fluorescent protein (e.g., gpt, GFP, mCherry). In some embodiments, the VACVΔB2R virus encompasses a recombinant vaccinia virus that does not express a functional thymidine kinase (TK) protein. In some embodiments, a specific gene of interest (e.g., OX40L, hFlt3L) is inserted into the TK locus (e.g., position 80,962 to 81,032 of SEQ ID NO: 2), splitting the TK gene and obliterating it (“VACVΔB2R-OX40L-TK(−)”; “VACVΔB2R-hFlt3L-TK(−)”). In some embodiments, VACVΔB2R encompasses a recombinant vaccinia virus in which all or a majority of the B2R gene sequence is replaced by a first specific gene of interest (e.g., hFlt3L) and a second gene of interest (e.g., OX40L) is inserted into the TK locus (“VACVΔB2R-hFlt3L-TK(−)-OX40L”). In some embodiments, the engineered VACVΔB2R viruses of the present technology are modified to express at least one heterologous gene, such as any one or more of hOX40L, hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or include at least one viral gene mutation or deletion, such as any one or more of the following vaccinia viral deletions: E3L (ΔE3L); E3LΔ83N; C7L (ΔC7L); B19R (B18R; ΔWR200); E5R; K7R; C12L (IL18BP); B8R; B14R; N1L; C11R; K1L; M1L; N2L; and/or WR199. As another example, in some embodiments, the TK locus of the vaccinia genome is modified through homologous recombination to express both the heavy and light chain of an antibody, such as anti-CTLA-4, wherein the coding sequences of the heavy chain and light chain are separated by a cassette including a furin cleavage site followed by a 2A peptide (Pep2A) sequence to produce VACV-TK(−)-anti-CTLA-4. In some embodiments, the VACV-TK(−)-anti-CTLA-4 genome is further modified to comprise a deletion of B2R, in which all or a majority of the B2R gene sequence is replaced by a first specific gene of interest (e.g., hFlt3L) and a second specific gene of interest (e.g., OX40L), wherein the coding sequences of the first and second specific genes of interest are separated by a cassette including a furin cleavage site followed by a 2A peptide (Pep2A) sequence. In other embodiments, the VACVΔB2R viruses of the present technology do not express any further heterologous genes and/or do not include any additional viral gene mutations or deletions other than those specifically indicated in the name of the virus.

The term “MYXVΔM31R,” is used herein to refer to a myxoma mutant virus or vaccine comprising the virus in which the M31R gene is not expressed, expressed at levels so low as to have no effect, or the expressed protein is non-functional (e.g., is a null-mutation).

Myxoma virus M31R is the ortholog of the vaccinia virus E5R. As used herein, “MYXVΔM31R” encompasses a recombinant myxoma virus (MYXV) that does not express a functional M31R protein. In some embodiments, the ΔM31R mutant includes a heterologous sequence in place of all or a majority of the M31R gene sequence. For example, as used herein, “MYXVΔM31R” encompasses a recombinant myxoma virus nucleic acid sequence, wherein the nucleic acid sequence corresponding to the position of M31R in the MYXV genome (e.g., position 30,138 to 31,319 of the MYXV genome) is replaced with a heterologous nucleic acid sequence comprising an open reading frame that encodes a specific gene of interest (SG), such as human OX40L (“MYXVΔM31R-OX40L”) or human Fms-like tyrosine kinase 3 ligand (hFlt3L) gene (“MYXVΔM31R-hFlt3L”). In some embodiments, MYXVΔM31R encompasses a recombinant MYXV wherein the M31R locus is modified to express one or more heterologous genes. For example, in some embodiments, MYXVΔM31R encompasses a recombinant MYXV in which all or a majority of the M31R gene sequence is replaced by a first specific gene of interest (e.g., hFtl3L) and a second specific gene of interest (e.g., OX40L), wherein the coding sequences of the first and second specific genes of interest are separated by a cassette including a furin cleavage site followed by a 2A peptide (Pep2A) sequence, thereby forming a recombinant virus such as “MYXVΔM31R-hFlt3L-OX40L.” In some embodiments, the heterologous nucleic acid sequence further comprises an open reading frame that encodes a selectable marker. In some embodiments, the selectable marker is a fluorescent protein (e.g., gpt, GFP, mCherry). In some embodiments, the MYXVΔM31R virus encompasses a recombinant myxoma virus that does not express a functional thymidine kinase (TK) protein. In some embodiments, a specific gene of interest (e.g., OX40L, hFlt3L) is inserted into the TK locus (e.g., position 57,797 to 58,333 of the myxoma genome), splitting the TK gene and obliterating it (“MYXVΔM31R-OX40L-TK(−)”; “MYXVΔM31R-hFlt3L-TK(−)”). In some embodiments, MYXVΔM31R encompasses a recombinant myxoma virus in which all or a majority of the M31R gene sequence is replaced by a first specific gene of interest (e.g., hFlt3L) and a second gene of interest (e.g., OX40L) is inserted into the TK locus (“MYXVΔM31R-hFlt3L-TK(−)-OX40L”). In some embodiments, the engineered MYXVΔM31R viruses of the present technology are modified to express at least one heterologous gene, such as any one or more of hOX40L, hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or include at least one viral gene mutation or deletion, such as any one or more of the following myxoma orthologs of vaccinia viral deletions: E3L (ΔE3L); E3LΔ83N; C7L (ΔC7L); B2R (ΔB2R), B19R (B18R; ΔWR200); K7R; C12L (IL18BP); B8R; B14R; N1L; C11R; K1L; M1L; N2L; and/or WR199. In other embodiments, the MYXVΔM31R viruses of the present technology do not express any further heterologous genes and/or do not include any additional viral gene mutations or deletions other than those specifically indicated in the name of the virus.

The term “MYXVΔM63R,” is used herein to refer to a myxoma mutant virus or vaccine comprising the virus in which the M63R gene is not expressed, expressed at levels so low as to have no effect, or the expressed protein is non-functional (e.g., is a null-mutation). As used herein, “MYXVΔM63R” encompasses a recombinant myxoma virus (MYXV) that does not express a functional M63R protein. In some embodiments, the ΔM63R mutant includes a heterologous sequence in place of all or a majority of the M63R gene sequence. For example, as used herein, “MYXVΔM63R” encompasses a recombinant myxoma virus nucleic acid sequence, wherein the nucleic acid sequence corresponding to the position of M63R in the MYXV genome is replaced with a heterologous nucleic acid sequence comprising an open reading frame that encodes a specific gene of interest (SG), such as human OX40L (“MYXVΔM63R-OX40L”) or human Fms-like tyrosine kinase 3 ligand (hFlt3L) gene (“MYXVΔM63R-hFlt3L”). In some embodiments, MYXVΔM63R encompasses a recombinant MYXV wherein the M63R locus is modified to express one or more heterologous genes. For example, in some embodiments, MYXVΔM63R encompasses a recombinant MYXV in which all or a majority of the M63R gene sequence is replaced by a first specific gene of interest (e.g., hFtl3L) and a second specific gene of interest (e.g., OX40L), wherein the coding sequences of the first and second specific genes of interest are separated by a cassette including a furin cleavage site followed by a 2A peptide (Pep2A) sequence, thereby forming a recombinant virus such as “MYXVΔM63R-hFlt3L-OX40L.” Additionally or alternatively, in some embodiments, the heterologous nucleic acid sequence comprises an open reading frame that encodes a selectable marker. In some embodiments, the selectable marker is a fluorescent protein (e.g., gpt, GFP, mCherry). In some embodiments, the MYXVΔM63R virus encompasses a recombinant myxoma virus that does not express a functional thymidine kinase (TK) protein. In some embodiments, a specific gene of interest (e.g., OX40L, hFlt3L) is inserted into the TK locus (e.g., position 57,797 to 58,333 of the myxoma genome), splitting the TK gene and obliterating it (“MYXVΔM63R-OX40L-TK(−)”; “MYXVΔM63R-hFlt3L-TK(−)”). In some embodiments, MYXVΔM63R encompasses a recombinant myxoma virus in which all or a majority of the M63R gene sequence is replaced by a first specific gene of interest (e.g., hFlt3L) and a second gene of interest (e.g., OX40L) is inserted into the TK locus (“MYXVΔM63R-hFlt3L-TK(−)-OX40L”). In some embodiments, the engineered MYXVΔM63R viruses of the present technology are modified to express at least one heterologous gene, such as any one or more of hOX40L, hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or include at least one viral gene mutation or deletion, such as any one or more of the following myxoma orthologs of vaccinia viral deletions: E3L (ΔE3L); E3LΔ83N; C7L (ΔC7L); B2R (ΔB2R), B19R (B18R; ΔWR200); K7R; C12L (IL18BP); B8R; B14R; N1L; C11R; K1L; M1L; N2L; and/or WR199. In some embodiments, MYXVΔM63R is further engineered to comprise additional myxoma gene deletions (e.g., ΔM31R, ΔM62R, and/or ΔM64R). In other embodiments, the MYXVΔM63R viruses of the present technology do not express any further heterologous genes and/or do not include any additional viral gene mutations or deletions other than those specifically indicated in the name of the virus.

The term “MYXVΔM64R,” is used herein to refer to a myxoma mutant virus or vaccine comprising the virus in which the M64R gene is not expressed, expressed at levels so low as to have no effect, or the expressed protein is non-functional (e.g., is a null-mutation). As used herein, “MYXVΔM64R” encompasses a recombinant myxoma virus (MYXV) that does not express a functional M64R protein. In some embodiments, the ΔM64R mutant includes a heterologous sequence in place of all or a majority of the M64R gene sequence. For example, as used herein, “MYXVΔM64R” encompasses a recombinant myxoma virus nucleic acid sequence, wherein the nucleic acid sequence corresponding to the position of M64R in the MYXV genome is replaced with a heterologous nucleic acid sequence comprising an open reading frame that encodes a specific gene of interest (SG), such as human OX40L (“MYXVΔM64R-OX40L”) or human Fms-like tyrosine kinase 3 ligand (hFlt3L) gene (“MYXVΔM64R-hFlt3L”). In some embodiments, MYXVΔM64R encompasses a recombinant MYXV wherein the M64R locus is modified to express one or more heterologous genes. For example, in some embodiments, MYXVΔM64R encompasses a recombinant MYXV in which all or a majority of the M64R gene sequence is replaced by a first specific gene of interest (e.g., hFtl3L) and a second specific gene of interest (e.g., OX40L), wherein the coding sequences of the first and second specific genes of interest are separated by a cassette including a furin cleavage site followed by a 2A peptide (Pep2A) sequence, thereby forming a recombinant virus such as “MYXVΔM64R-hFlt3L-OX40L.” Additionally or alternatively, in some embodiments, the heterologous nucleic acid sequence comprises an open reading frame that encodes a selectable marker. In some embodiments, the selectable marker is a fluorescent protein (e.g., gpt, GFP, mCherry). In some embodiments, the MYXVΔM64R virus encompasses a recombinant myxoma virus that does not express a functional thymidine kinase (TK) protein. In some embodiments, a specific gene of interest (e.g., OX40L, hFlt3L) is inserted into the TK locus (e.g., position 57,797 to 58,333 of the myxoma genome), splitting the TK gene and obliterating it (“MYXVΔM64R-OX40L-TK(−)”; “MYXVΔM64R-hFlt3L-TK(−)”). In some embodiments, MYXVΔM64R encompasses a recombinant myxoma virus in which all or a majority of the M64R gene sequence is replaced by a first specific gene of interest (e.g., hFlt3L) and a second gene of interest (e.g., OX40L) is inserted into the TK locus (“MYXVΔM64R-hFlt3L-TK(−)-OX40L”). In some embodiments, the engineered MYXVΔM64R viruses of the present technology are modified to express at least one heterologous gene, such as any one or more of hOX40L, hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or include at least one viral gene mutation or deletion, such as any one or more of the following myxoma orthologs of vaccinia viral deletions: E3L (ΔE3L); E3LΔ83N; C7L (ΔC7L); B2R (ΔB2R), B19R (B18R; ΔWR200); K7R; C12L (IL18BP); B8R; B14R; N1L; C11R; K1L; M1L; N2L; and/or WR199. In some embodiments, MYXVΔM64R is further engineered to comprise additional myxoma gene deletions (e.g., ΔM31R, ΔM62R, and/or ΔM63R). In other embodiments, the MYXVΔM64R viruses of the present technology do not express any further heterologous genes and/or do not include any additional viral gene mutations or deletions other than those specifically indicated in the name of the virus.

As used herein, “oncolytic virus” refers to a virus that preferentially infects cancer cells, replicates in such cells, and induces lysis of the cancer cells through its replication process. Nonlimiting examples of naturally occurring oncolytic viruses include vesicular stomatitis virus, reovirus, as well as viruses engineered to be oncoselective such as adenovirus, Newcastle disease virus and herpes simplex virus (See, e.g., Nemunaitis, J. Invest. New Drugs 17(4):375-86 (1999); Kirn, D H et al., Nat. Rev. Cancer 9(1):64-71(2009); Kirn et al., Nat. Med. 7:781 (2001); Coffey et al., Science 282:1332 (1998)). Vaccinia virus infects many types of cells but replicates preferentially in tumor cells due to the fact that tumor cells have a metabolism that favors replication, exhibit activation of certain pathways that also favor replication and create an environment that evades the innate immune system, which also favors viral replication.

As used herein, “parenteral,” when used in the context of administration of a therapeutic substance or composition, includes any route of administration other than administration through the alimentary tract. Particularly relevant for the methods disclosed herein are intravenous (including, for example, through the hepatic portal vein for hepatic delivery), intratumoral, or intrathecal administration.

The terms “pharmaceutically acceptable excipient,” “pharmaceutically acceptable diluent,” “pharmaceutically acceptable carrier,” or “pharmaceutically acceptable adjuvant” refer to an excipient, diluent, carrier, and/or adjuvant useful in preparing a pharmaceutical composition that is generally safe, non-toxic and neither biologically nor otherwise undesirable, and includes an excipient, diluent, carrier, and adjuvant that is acceptable for pharmaceutical use. “A pharmaceutically acceptable excipient, diluent, carrier and/or adjuvant” as used in the specification and claims includes one and more such excipients, diluents, carriers, and adjuvants.

As used herein, “prevention,” “prevent,” or “preventing” of a disorder or condition refers to one or more compounds that, in a statistical sample, reduces the occurrence of the disorder or condition in the treated sample relative to an untreated control sample, or delays the onset of one or more symptoms of the disorder or condition relative to the untreated control sample.

As used herein, the term “recombinant” when used with reference, e.g., to a virus, or cell, or nucleic acid, or protein, or vector, indicates that the virus, cell, nucleic acid, protein or vector, has been modified by the introduction of a heterologous nucleic acid or protein or the alteration of a native nucleic acid or protein, or that the material is derived from a virus or cell so modified. Thus, for example, recombinant viruses or cells express genes that are not found within the native (non-recombinant) form of the virus or cell or express native genes that are otherwise abnormally expressed, under expressed or not expressed at all.

As used herein, “solid tumor” refers to all neoplastic cell growth and proliferation, and all pre-cancerous and cancerous cells and tissues, except for hematologic cancers such as lymphomas, leukemias, and multiple myeloma. Examples of solid tumors include, but are not limited to: soft tissue sarcoma, such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor and other bone tumors (e.g., osteosarcoma, malignant fibrous histiocytoma), leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, brain/CNS tumors (e.g., astrocytoma, glioma, glioblastoma, childhood tumors, such as atypical teratoid/rhabdoid tumor, germ cell tumor, embryonal tumor, ependymoma) medulloblastoma, craniopharyngioma, ependymoma, pinealoma, hemangioblastoma, acoustic neuroma, oligodendroglioma, meningioma, melanoma, neuroblastoma, and retinoblastoma. Some of the most common solid tumors for which the compositions and methods of the present disclosure would be useful include: head-and-neck cancer, rectal adenocarcinoma, glioma, medulloblastoma, urothelial carcinoma, pancreatic adenocarcinoma, uterine (e.g., endometrial cancer, fallopian tube cancer) ovarian cancer, cervical cancer prostate adenocarcinoma, non-small cell lung cancer (squamous and adenocarcinoma), small cell lung cancer, melanoma, breast carcinoma, bladder cancer, ductal carcinoma in situ, renal cell carcinoma, and hepatocellular carcinoma, adrenal tumors (e.g., adrenocortical carcinoma), esophageal, eye (e.g., melanoma, retinoblastoma), gallbladder, gastrointestinal, Wilms' tumor, heart, head and neck, laryngeal and hypopharyngeal, oral (e.g., lip, mouth, salivary gland), nasopharyngeal, neuroblastoma, peritoneal, pituitary, Kaposi's sarcoma, small intestine, stomach, testicular, thymus, thyroid, parathyroid, vaginal tumor, and the metastases of any of the foregoing.

As used herein, the terms “subject,” “individual,” or “patient” are used interchangeably herein, and can be an individual organism, a vertebrate, a mammal, or a human. In some embodiments, “subject” means any animal (mammalian, human, or other) patient that can be afflicted with cancer and when thus afflicted is in need of treatment. In some embodiments, “subject” means human.

As used herein, a “synergistic therapeutic effect” in some embodiments reflects a greater-than-additive therapeutic effect that is produced by a combination of at least two agents, and which exceeds that which would otherwise result from the individual administration of the agents. In some embodiments, a “synergistic therapeutic effect” reflects an enhanced therapeutic effect that is produced by a combination of at least two agents relative to the individual administration of the agents. For example, lower doses of one or more agents may be used in treating a disease or disorder, resulting in increased therapeutic efficacy and decreased side-effects.

“Treating,” “treat,” “treated,” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission. In some embodiments, “inhibiting,” means reducing or slowing the growth of a tumor. In some embodiments, the inhibition of tumor growth may be, for example, by 5% or more, 10% or more, 20% or more, 30% or more, 40% or more, 50% or more, 60% or more, 70% or more, 80% or more, or 90% or more. In some embodiments, the inhibition may be complete.

It is also to be appreciated that the various modes of treatment or prevention of medical diseases and conditions as described are intended to mean “substantial,” which includes total but also less than total treatment or prevention, and wherein some biologically or medically relevant result is achieved.

As used herein, “tumor immunity” refers to one or more processes by which tumors evade recognition and clearance by the immune system. Thus, as a therapeutic concept, tumor immunity is “treated” when such evasion is attenuated or eliminated, and the tumors are recognized and attacked by the immune system (the latter being termed herein “anti-tumor immunity”). An example of tumor recognition is tumor binding, and examples of tumor attack are tumor reduction (in number, size, or both) and tumor clearance.

As used herein, “T-cell” refers to a thymus derived lymphocyte that participates in a variety of cell-mediated adaptive immune reactions. As used herein, “effector T-cell” includes helper, killer, and regulatory T-cells.

As used herein, “helper T-cell” refers to a CD4⁺ T-cell; helper T-cells recognize antigen bound to MHC Class II molecules. There are at least two types of helper T-cells, Th1 and Th2, which produce different cytokines.

As used herein, “cytotoxic T-cell” refers to a T-cell that usually bears CD8 molecular markers on its surface (CD8⁺) and that functions in cell-mediated immunity by destroying a target T-cell having a specific antigenic molecule on its surface. Cytotoxic T-cells also release Granzyme, a serine protease that can enter target T-cells via the perforin-formed pore and induce apoptosis (cell death). Granzyme serves as a marker of cytotoxic phenotype. Other names for cytotoxic T-cell include CTL, cytolytic T-cell, cytolytic T lymphocyte, killer T-cell, or killer T lymphocyte. Targets of cytotoxic T-cells may include virus-infected cells, cells infected with bacterial or protozoal parasites, or cancer cells. Most cytotoxic T-cells have the protein CD8 present on their cell surfaces. CD8 is attracted to portions of the Class I MHC molecule. Typically, a cytotoxic T-cell is a CD8⁺ cell.

As used herein, “tumor-infiltrating leukocytes” refers to white blood cells of a subject afflicted with a cancer (such as melanoma), that are resident in or otherwise have left the circulation (blood or lymphatic fluid) and have migrated into a tumor.

As used herein, “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “operatively linked,” “under control,” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or inhibitory RNA (e.g., shRNA, miRNA) from a transcribed gene. A non-limiting example of a pCB-OX40L-gpt vector according to the present technology is set forth in SEQ ID NO: 3. A non-limiting example of a pUC57-hFlt3L-GFP vector according to the present technology is set forth in SEQ ID NO: 4. A non-limiting example of a pUC57-delC7-hOX40L-mCherry vector is set forth in SEQ ID NO: 5.

The term “virulence” as used herein to refer to the relative ability of a pathogen to cause disease. The term “attenuated virulence” or “reduced virulence” is used herein to refer to a reduced relative ability of a pathogen to cause disease.

### II. Immune System and Cancer

Malignant tumors are inherently resistant to conventional therapies and present significant therapeutic challenges. Immunotherapy has become an evolving area of research and an additional option for the treatment of certain types of cancers. The immunotherapy approach rests on the rationale that the immune system may be stimulated to identify tumor cells and target them for destruction.

Numerous studies support the importance of the differential presence of immune system components in cancer progression (Jochems et al., Exp. Biol. Med. 236(5):567-579 (2011)). Clinical data suggest that high densities of tumor-infiltrating lymphocytes are linked to improved clinical outcome (Mlecnik et al., Cancer Metastasis Rev. 30:5-12, (2011)). The correlation between a robust lymphocyte infiltration and patient survival has been reported in various types of cancer, including melanoma, ovarian, head and neck, breast, bladder, urothelial, colorectal, lung, hepatocellular, gallbladder, and esophageal cancer (Angell et al., Current Opinion in Immunology 25:1-7, (2013)). Tumor immune infiltrates include macrophages, dendritic cells (DC), monocytes, neutrophils, natural killer (NK) cells, naïve and memory lymphocytes, B cells and effector T-cells (T lymphocytes), primarily responsible for the recognition of antigens expressed by tumor cells and subsequent destruction of the tumor cells by cytotoxic T-cells.

Despite presentation of antigens by cancer cells and the presence of immune cells that could potentially react against tumor cells, in many cases the immune system does not get activated or is affirmatively suppressed. Key to this phenomenon is the ability of tumors to protect themselves from immune response by coercing cells of the immune system to inhibit other cells of the immune system. Tumors develop a number of immunomodulatory mechanisms to evade antitumor immune responses. For example, tumor cells secrete immune inhibitory cytokines (such as TGF-β) or induce immune cells, such as CD4⁺ T regulatory cells and macrophages, in tumor lesions to secrete these cytokines. Tumors also have the ability to bias CD4⁺ T-cells to express the regulatory phenotype. The overall result is impaired T-cell responses and impaired induction of apoptosis or reduced anti-tumor immune capacity of CD8⁺ cytotoxic T-cells. Additionally, tumor-associated altered expression of MHC class I on the surface of tumor cells makes them “invisible” to the immune response (Garrido et al. Cancer Immunol. Immunother. 59(10):1601-1606 (2010)). Inhibition of antigen-presenting functions and dendritic cell (DC) additionally contributes to the evasion of anti-tumor immunity (Gerlini et al. Am. J. Pathol. 165(6):1853-1863 (2004)).

Moreover, the local immunosuppressive nature of the tumor microenvironment, along with immune editing, can lead to the escape of cancer cell subpopulations that do not express the target antigens. Thus, finding an approach that would promote the preservation and/or restoration of anti-tumor activities of the immune system would be of considerable therapeutic benefit.

Immune checkpoints have been implicated in the tumor-mediated downregulation of anti-tumor immunity and used as therapeutic targets. It has been demonstrated that T-cell dysfunction occurs concurrently with an induced expression of the inhibitory receptors, CTLA-4 and programmed death 1 polypeptide (PD-1), members of the CD28 family of receptors. PD-1 is an inhibitory member of the CD28 family of receptors that in addition to PD-1 includes CD28, CTLA-4, ICOS, and BTLA. However, while promise regarding the use of immunotherapy in the treatment of melanoma has been underscored by the clinical use and even regulatory approval of anti-CTLA-4 (ipilimumab) and anti-PD-1 drugs (e.g., pembrolizumab and nivolumab), the response of patients to these immunotherapies has been limited. Clinical trials, focused on blocking these inhibitory signals in T-cells (e.g., CTLA-4, PD-1, and the ligand of PD-1, PD-L1), have shown that reversing T-cell suppression is critical for successful immunotherapy (Sharma et al., Science 348(6230):56-61 (2015); Topalian et al., Curr. Opin. Immunol. 24(2):202-217 (2012)). These observations highlight the need for development of novel therapeutic approaches for harnessing the immune system against cancer.

### III. Poxviruses: Vaccinia Virus (VACV), Modified Vaccinia Ankara (MVA) Virus, and Myxoma Virus (MYXV)

Poxviruses, such as engineered vaccinia viruses, are in the forefront as oncolytic therapy for metastatic cancers (Kirn et al., Nature Review Cancer 9:64-71 (2009)). Vaccinia viruse (VACV), a member of the Poxvirus family, is a large DNA virus, which has a rapid life cycle and efficient hematogenous spread to distant tissues. Poxviruses are well-suited as vectors to express multiple transgenes in cancer cells and thus to enhance therapeutic efficacy (Breitbach et al., Current pharmaceutical biotechnology 13:1768-1772 (2012)). Preclinical studies and clinical trials have demonstrated efficacy of using oncolytic vaccinia viruses and other poxviruses for treatment of advanced cancers refractory to conventional therapy (Park et al., Lacent Oncol. 9:533-542 (2008); Kirn et al., PLoS Med 4:e353 (2007); Thorne et al., J. Clin. Invest. 117:3350-3358 (2007)). Poxvirus-based oncolytic therapy has the advantage of killing cancer cells through a combination of cell lysis, apoptosis, and necrosis. It also triggers innate immune sensing pathway that facilitates the recruitment of immune cells to the tumors and the development of anti-tumor adaptive immune responses. The current oncolytic vaccinia strains in clinical trials (JX-594, for example) are replicative strains. They use wild-type vaccinia with deletion of thymidine kinase to enhance tumor selectivity, and with expression of transgenes such as granulocyte macrophage colony stimulating factor (GM-CSF) to stimulate immune responses (Breitbach et al., Curr. Pharm. Biotechnol. 13:1768-1772 (2012)). Many studies have shown, however, that wild-type vaccinia has immune suppressive effects on antigen presenting cells (APCs) (Engelmayer et al., J. Immunol. 163:6762-6768 (1999); Jenne et al., Gene Therapy 7:1575-1583 (2000); P. Li et al., J. Immunol. 175:6481-6488 (2005); Deng et al., J. Virol. 80:9977-9987 (2006)), and thus adds to the immunosuppressive and immunoevasive effects of tumors themselves.

The vaccinia virus (Western Reserve strain; WR) genome sequence is set forth in SEQ ID NO: 2, and is given by GenBank Accession No. AY243312.1.

Modified Vaccinia Ankara (MVA) virus is also a member of the Poxvirus family. MVA was generated by approximately 570 serial passages on chicken embryo fibroblasts (CEF) of the Ankara strain of vaccinia virus (CVA) (Mayr et al., Infection 3:6-14 (1975)). As a consequence of these long-term passages, the resulting MVA virus contains extensive genome deletions and is highly host cell restricted to avian cells (Meyer et al., J. Gen. Virol. 72:1031-1038 (1991)). It was shown in a variety of animal models that the resulting MVA is significantly avirulent (Mayr et al., Dev. Biol. Stand. 41:225-34 (1978)).

The safety and immunogenicity of MVA has been extensively tested and documented in clinical trials, particularly against the human smallpox disease. These studies included over 120,000 individuals and have demonstrated excellent efficacy and safety in humans. Moreover, compared to other vaccinia based vaccines, MVA has weakened virulence (infectiousness) while it triggers a good specific immune response. Thus, MVA has been established as a safe vaccine vector, with the ability to induce a specific immune response.

Due to the above mentioned characteristics, MVA became an attractive candidate for the development of engineered MVA vectors, used for recombinant gene expression and vaccines. As a vaccine vector, MVA has been investigated against numerous pathological conditions, including HIV, tuberculosis and malaria, as well as cancer (Sutter et al., Curr. Drug Targets Infect. Disord. 3:263-271(2003); Gomez et al., Curr. Gene Ther. 8:97-120 (2008)).

It has been demonstrated that MVA infection of human monocyte-derived dendritic cells (DC) causes DC activation, characterized by the upregulation of co-stimulatory molecules and secretion of proinflammatory cytokines (Drillien et al., J. Gen. Virol. 85:2167-2175 (2004)). In this respect, MVA differs from standard wild type vaccinia virus (WT-VAC), which fails to activate DCs. Dendritic cells can be classified into two main subtypes: conventional dendritic cells (cDCs) and plasmacytoid dendritic cells (pDCs). The former, especially the CD103⁺/CD8α⁺ subtype, are particularly adapted to cross-presenting antigens to T-cells; the latter are strong producers of Type I IFN.

Viral infection of human cells results in activation of an innate immune response (the first line of defense) mediated by type I interferons, notably interferon-alpha (a). This normally leads to activation of an immunological “cascade,” with recruitment and proliferation of activated T-cells (both CTL and helper) and eventually with antibody production. However, viruses express factors that dampen immune responses of the host. MVA is a better immunogen than WT-VAC and replicates poorly in mammalian cells. (See, e.g., Brandler et al., J. Virol. 84:5314-5328 (2010)).

However, MVA is not entirely non-replicative and contains some residual immunosuppressive activity. Nevertheless, MVA has been shown to prolong survival of treated subjects.

The MVA genome sequence is set forth in SEQ ID NO: 1 and is given by GenBank Accession No. U94848.1.

Myxoma virus (MYXV) is the prototypic member of the Leporipoxvirus genus within the Poxviridae family. The MYXV Lausanne strain genome (given by, e.g., GenBank Accession No. AF170726.2) is 161.8 kbp in size, encoding about 171 genes. The central region of the genome encodes less than 100 genes that are highly conserved in all poxviruses while the terminal genomic regions are enriched for more unique genes that encode immunomodulatory and host-interactive factors that are involved in subverting the host immune system and other anti-viral responses. Myxoma virus exhibits a very restricted host range and is only pathogenic to European rabbits. Despite its narrow host range in nature, MYXV has been shown to productively infect various classes of human cancer cells. Attractive features of MYXV as an oncolytic agent include its ability to productively infect various human cancer cells and its consistent safety in all non-rabbit hosts tested, including mice and humans. In some embodiments, the myxoma virus is derived from strain Lausanne.

### V. Vaccinia Virus C7 Protein and MVA or Vaccinia Virus Comprisinm Deletion of C7 (MVAΔC7L, VACVΔC7L), or Myxoma Virus Comprising Deletion of Myxoma C7 Orthologs

Vaccinia virus C7 protein is an important host range factor for vaccinia virus life cycle in mammalian cells. C7L homologs are present in almost all of the poxviruses that infect mammalian hosts. Deletion of both host range gene C7L and K1L renders the virus incapable of replication in human cells (Perkus et al., Virology, 1990). The mutant virus deficient of both K1L and C7L gains its ability to replicate in human HeLa cells when SAMD9 is knocked-out (Sivan et al., MBio, 2015). Both K1 and C7 have been found to interact with SAMD9 (Sivan et al., MBio, 2015). Overexpression of IRF1 leads to host restriction of C7L and K1L double deleted vaccinia virus (Meng et al., Journal of Virology, 2012). Both C7 and K1 interact with SAMD9 in vitro (Sivan et al., MBio. 2015). Whether C7 directly modulates IFN production or signaling is unknown. Type I IFN plays an important role in host defense of viral infection, and yet, the role of C7 in immune modulation of the IFN pathway is unclear.

Without wishing to be bound by theory, it is thought that vaccinia C7 is an inhibitor of type I IFN induction and IFN signaling. TANK Binding Kinase 1 (TBK1) is a serine/threonine kinase that plays a critical role in the induction of innate immune responses to various pathogen-associated molecular patterns (PAMPs), including nucleic acids. On the one hand, RIG-I-like receptors such as RIG-I and MDA5, which detect 5′ triphosphate RNA and dsRNA, respectively, interact with a mitochondrial protein IPS-1 or MAVS, leading to the activation and phosphorylation of TBK1. Endosomal dsRNA binds to Toll-like receptor 3 (TLR3), which results in the recruitment of TRIF and TRAF3 and activation of TBK1. On the other hand, cytosolic DNA can be detected by the cytosolic DNA sensor cyclic GMP-AMP synthase (cGAS), which leads to the production of cyclic GMP-AMP (cGAMP). cGAMP, in turn, binds to the endoplasmic reticulum (ER)-localized adaptor STING, leading to the recruitment and activation of TBK1. TBK1 phosphorylates transcription factor IRF3, which translocates to the nucleus to activate IFNB gene expression. Without wishing to be bound by theory, it is believed that C7 inhibits IFNB induction by various stimuli, including RNA virus, DNA virus, poly (I:C), immunostimulatory DNA (ISD). C7 may exert its inhibitory effect at the level of TBK1/IRF3 complex. Once secreted, type I IFN binds to IFNAR, which leads to the activation of the JAK/STAT signaling pathway. Phosphorylated STAT1 and STAT2 translocate to the nucleus, where together with IRF9, they activate the expression of IFN-stimulated genes (ISGs). Without wishing to be bound by theory, it is believed that in addition to its ability to inhibit IFNB induction, C7 can also block IFNAR signaling through its interaction of STAT2, thereby preventing IFN-β-induced STAT2 phosphorylation. Without wishing to be bound by theory, it is believed that vaccinia C7 has dual inhibitory role of type I IFN production and signaling. Previous studies have shown that the deletion of C7L from WT vaccinia (VACVΔC7L) results in the attenuation of the virus and deletion of C7L from MVA (MVAΔC7L) leads to enhanced immunostimulatory functions compared with MVA.

Ectopic C7 expression has been shown to block STING, TBK1, or IRF3-induced IFNB and ISRE (interferon stimulated response element) promoter activation. Murine or human macrophage cell lines that overexpress C7 have been shown to have blunted innate immune responses to DNA or RNA stimuli, or the infection of DNA or RNA viruses. It has also been shown that overexpression of C7 attenuates ISG gene expression induced by IFN-β treatment. MVA with deletion of C7L (MVAΔC7L) infection of cDCs has been shown to induce higher levels of type I IFN than MVA. C7 has been shown to block IFN-β-induced Janus kinase/signal transducer and activator of transcription (JAK/STAT) signaling pathway via preventing Stat2 phosphorylation. C7 has been shown to directly interact with Stat2 as demonstrated by co-immunoprecipitation studies.

An illustrative full-length vaccinia virus C7 host range protein, given by GenBank Accession No. AAB96405.1 (SEQ ID NO: 6) is provided below.
1 mgighefdii ingdialrnl glhkgdnygc klkiisndyk klkfrfiirp dwseidevkg
61 ltvfannyav kvnkvddtfy yviyeavihl ynkkteiliy sddenelfkh yypyislnmi
121 skkykvkeen ysspyiehpl ipyrdyesmd

Myxoma virus has three C7 orthologs, M62, M63, M64. Myxoma M64 shares similar structure features with vaccinia C7, despite having only 23% sequence identity. Myxoma M62 can rescue replication defects of VACVΔK1LΔC7L in human cells. Myxoma M63 deletion results in a recombinant virus that is non-replicative in rabbit cells. In some embodiments, the technology of the present disclosure provides an engineered myxoma virus such as MYXVΔM64R or MYXVΔM64R-hFlt3L-mOX40L.

### VI. OX40 Ligand (OX40L)

The OX40 ligand (OX40L) and its binding partner, tumor necrosis factor receptor OX40, are members of the TNFR/TNF superfamily and are expressed on activated CD4 and CD8 T-cells as well as a number of other lymphoid and non-lymphoid cells. The OX40L-OX40 interaction provides survival and activation signals for T-cells expressing OX40. OX40 additionally suppresses the differentiation and activity of Treg, further amplifying this process. OX40 and OX40L also regulate cytokine production from T-cells, antigen-presenting cells, NK cells, and NKT cells, and modulate cytokine receptor signaling. The OX40L of the MVAΔE3L-OX40L, MVAΔC7L-OX40L, MVAΔC7L-hFlt3L-OX40L, MVAΔE5R, VACVΔC7L-OX40L, VACVΔC7L-hFlt3L-OX40L, VACVΔE5R, and MYXVΔM31R recombinant viruses of the present technology can be either huOX40L or muOX40L.

Illustrative human OX40L (huOX40L) nucleic acid (SEQ ID NO: 7) and polypeptide sequences (SEQ ID NO: 8) are provided below.

huOX40L-ORF (SEQ ID NO: 7):
ATGGAAAGGG TCCAACCCCT GGAAGAGAAT GTGGGAAATG
CAGCCAGGCC AAGATTCGAG AGGAACAAGC TATTGCTGGT
GGCCTCTGTA ATTCAGGGAC TGGGGCTGCT CCTGTGCTTC
ACCTACATCT GCCTGCACTT CTCTGCTCTT CAGGTATCAC
ATCGGTATCC TCGAATTCAA AGTATCAAAG TACAATTTAC
CGAATATAAG AAGGAGAAAG GTTTCATCCT CACTTCCCAA
AAGGAGGATG AAATCATGAA GGTGCAGAAC AACTCAGTCA
TCATCAACTG TGATGGGTTT TATCTCATCT CCCTGAAGGG
CTACTTCTCC CAGGAAGTCA ACATTAGCCT TCATTACCAG
AAGGATGAGG AGCCCCTCTT CCAACTGAAG AAGGTCAGGT
CTGTCAACTC CTTGATGGTG GCCTCTCTGA CTTACAAAGA
CAAAGTCTAC TTGAATGTGA CCACTGACAA TACCTCCCTG
GATGACTTCC ATGTGAATGG CGGAGAACTG ATTCTTATCC
ATCAAAATCC TGGTGAATTC TGTGTCCTTT GA
huOX40L polypeptide
(SEQ ID NO: 8)
ME R V Q P L E E N V G N A A R P R F E R N K L L L
V A S V I Q G L G L L L C F T Y I C L H F S A L Q
V S H R Y P R I Q S I K V Q F T E Y K K E K G F I
L T S Q K E D E I MK V Q N N S V I I N C D G F Y L
I S L K G Y F S Q E V N I S L H Y Q K D E E P L F
Q L K K V R S V N S L MV A S L T Y K D K V Y L N V
T T D N T S L D D F H V N G G E L I L I H Q N P G
E F C V L Stop

Illustrative murine OX40L (muOX40L) nucleic acid (SEQ ID NO: 9) and polypeptide sequences (SEQ ID NO: 10) are provided below.

muOX40L-ORF (codon optimized)(SEQ ID NO: 9):
ATGGAGGGCGAGGGGGTCCAGCCTCTGGACGAGAACCTCGAAAACGGGTC
TCGCCCTCGCTTTAAATGGAAGAAGACTCTTAGGCTCGTTGTAAGCGGCA
TCAAGGGGGCCGGTATGTTGCTGTGCTTCATATATGTGTGTTTGCAACTT
AGCTCTTCACCTGCAAAAGACCCCCCCATACAACGCCTTCGGGGGGCTGT
GACCCGCTGTGAAGATGGTCAATTGTTTATTTCTTCTTACAAGAACGAGT
ATCAGACGATGGAAGTCCAGAATAACTCCGTAGTGATTAAGTGTGACGGA
CTGTACATCATCTACTTGAAAGGATCTTTTTTCCAGGAGGTCAAAATTGA
CCTCCACTTCAGGGAGGATCACAACCCTATCTCAATCCCTATGTTGAACG
ACGGCAGAAGAATCGTCTTTACTGTAGTCGCTTCACTGGCCTTCAAGGAT
AAGGTGTACTTGACCGTAAACGCTCCTGATACCTTGTGCGAGCATTTGCA
AATCAACGATGGAGAACTTATCGTTGTCCAACTCACACCAGGTTACTGTG
CTCCTGAGGGCAGTTATCACAGTACAGTGAACCAAGTCCCACTGTGA
muOX40L polypeptide (SEQ ID NO: 10):
ME G E G V Q P L D E N L E N G S R P R F K W K K T
L R L V V S G I K G A G ML L C F I Y V C L Q L S S
S P A K D P P I Q R L R G A V T R C E D G Q L F I
S S Y K N E Y Q T ME V Q N N S V V I K C D G L Y I
I Y L K G S F F Q E V K I D L H F R E D H N P I S
I P ML N D G R R I V F T V V A S L A F K D K V Y L
T V N A P D T L C E H L Q I N D G E L I V V Q L T
P G Y C A P E G S Y H S T V N Q V P L Stop

### VII. Human Fms-Like Tyrosine Kinase 3 Ligand (hFlt3L)

Human Fms-like tyrosine kinase 3 ligand (hFlt3L), a type I transmembrane protein that stimulates the proliferation of bone marrow cells, was cloned in 1994 (Lyman et al., 1994). The use of hFlt3L has been explored in various preclinical and clinical settings including stem cell mobilization in preparation for bone marrow transplantation, cancer immunotherapy such as expansion of dendritic cells, as well as a vaccine adjuvant. Recombinant human Flt3L (rhuFlt3L) has been tested in more than 500 human subjects and is bioactive, safe, and well-tolerated. Much progress has been made in understanding the critical role of the growth factor Flt3L in the development of DC subsets, including CD8α⁺/CD103⁺ DCs and pDCs.

CD103⁺/CD8α⁺ DCs are required for spontaneous cross-priming of tumor antigen-specific CD8⁺ T-cells. It has been reported that CD103⁺ DCs are sparsely present within the tumors and they compete for tumor antigens with abundant tumor-associated macrophages. CD103⁺ DCs are uniquely capable of stimulating naïve as well as activated CD8⁺ T-cells and are critical for the success of adoptive T-cell therapy (Broz, et al. Cancer Cell, 26(5):638-52 (2014)). Spranger et al. reported that the activation of oncogenic signaling pathway WNT/β-catenin leads to reduction of CD103⁺ DCs and anti-tumor T-cells within the tumors (Spranger et al., 2015). Intratumoral delivery of Flt3L-cultured bone marrow derived dendritic cells (BMDCs) leads to responsiveness to the combination of anti-CTLA-4 and anti-PD-L1 immunotherapy (Spranger et al., 2015). Systemic administration of Flt3L, a growth factor for CD103⁺ DCs, and intratumor injection of poly I:C (TLR3 agonist) expanded and activated the CD103⁺ DC populations within the tumors and overcame resistance or enhanced responsiveness to immunotherapy in a murine melanoma and MC38 colon cancer models.

The recent discovery of tumor neoantigens in various solid tumors indicates that solid tumors harbor unique neoantigens that usually differ from person to person (Castle et al., Cancer Res 72:1081-1091 (2012); Schumacher et al., Science 348:69-74 (2015)). The genetically engineered or recombinant viruses disclosed herein do not exert their activity by expressing tumor antigens. Intratumoral delivery of the present genetically engineered or recombinant MVA viruses allows efficient cross-presentation of tumor neoantigens and generation of anti-tumor adaptive immunity within the tumors (and also extending systemically), and therefore leads to “in situ cancer vaccination” utilizing tumor differentiation antigens and neoantigens expressed by the tumor cells in mounting an immune response against the tumor.

Despite the presence of neoantigens generated by somatic mutations within tumors, the functions of tumor antigen-specific T-cells are often held in check by multiple inhibitory mechanisms (Mellman et al., Nature 480, 480-489 (2011)). For example, the up-regulation of cytotoxic T lymphocyte antigen 4 (CTLA-4) on activated T-cells can compete with T-cell co-stimulator CD28 to interact with CD80 (B71)/CD86 (B7.2) on dendritic cells (DCs), and thereby inhibit T-cell activation and proliferation. CTLA-4 is also expressed on regulatory T (Treg) cells and plays an important role in mediating the inhibitory function of Tregs (Wing et al., Science 322:271-275 (2008); Peggs, et al., J. Exp. Med. 206:1717-1725 (2009)). In addition, the expression of PD-L/PD-L2 on tumor cells can lead to the activation of the inhibitory receptor of the CD28 family, PD-1, leading to T-cell exhaustion. Immunotherapy utilizing antibodies against inhibitory receptors, such as CTLA-4 and programmed death 1 polypeptide (PD-1), have shown remarkable preclinical activities in animal studies and clinical responses in patients with metastatic cancers, and have been approved by the FDA for the treatment of metastatic melanoma, non-small cell lung cancer, as well as renal cell carcinoma (Leach et al., Science 271:1734-1746 (1996); Hodi et al., NEJM 363:711-723 (2010); Robert et al., NEJM 364:2517-2526 (2011); Topalian et al., Cancer Cell 27:450-461 (2012); Sharma et al., Science 348(6230):56-61 (2015)).

### VIII. ΔE3L and E3LΔ83N

Poxviruses are extraordinarily adept at evading and antagonizing multiple innate immune signaling pathways by encoding proteins that interdict the extracellular and intracellular components of those pathways (Seet et al. Annu. Rev. Immunol. 21:377-423 (2003)). Chief among the poxvirus antagonists of intracellular innate immune signaling is the vaccinia virus duel Z-DNA and dsRNA-binding protein E3, which can inhibit the PKR and NF-κB pathways (Cheng et al., Proc. Natl. Acad. Sci. USA 89:4825-4829 (1992); Deng et al., J. Virol. 80:9977-9987 (2006)) that would otherwise be activated by vaccinia virus infection. A mutant vaccinia virus lacking the E3L gene (ΔE3L) has a restricted host range, is highly sensitive to IFN, and has greatly reduced virulence in animal models of lethal poxvirus infection (Beattie et al., Virus Genes 1289-94 (1996); Brandt et al., Virology 333263-270 (2004)). Recent studies have shown that infection of cultured cell lines with ΔE3L virus elicits proinflammatory responses that are masked during infection with wild-type vaccinia virus (Deng et al., J. Virol. 80:9977-9987 (2006); Langland et al. J. Virol. 80:10083-10095). Infection of a mouse epidermal dendritic cell line with wild-type vaccinia virus attenuated proinflammatory responses to the TLR agonists lipopolysaccharide (LPS) and poly(I:C), an effect that was diminished by deletion of E3L. Moreover, infection of the dendritic cells with ΔE3L virus triggered NF-κB activation in the absence of exogenous agonists (Deng et al., J. Virol. 80:9977-9987 (2006)). Whereas wild-type vaccinia virus infection of murine keratinocytes does not induce the production of proinflammatory cytokines and chemokines, infection with ΔE3L virus does induce the production of IFN-β, IL-6, CCL4 and CCL5 from murine keratinocytes, which is dependent on the cytosolic dsRNA-sensing pathway mediated by the mitochondrial antiviral signaling protein (MAVS; an adaptor for the cytosolic RNA sensors RIG-I and MDA5) and the transcription factor IRF3 (Deng et al., J. Virol. 82(21):10735-10746 (2008)).

E3LΔ83N virus with deletion of the Z-DNA-binding domain is 1,000-fold more attenuated than wild-type vaccinia virus in an intranasal infection model (Brandt et al., 2001). E3LΔ83N also has reduced neurovirulence compared with wild-type vaccinia in an intra-cranial inoculation model (Brandt et al., 2005). A mutation within the Z-DNA binding domain of E3 (Y48A) resulting in decreased Z-DNA-binding leads to decreased neurovirulence (Kim et al., 2003). Although the N-terminal Z-DNA binding domain of E3 is important in viral pathogenesis, how it affects host innate immune sensing of vaccinia virus is not well understood. Myxoma virus but not wild-type vaccinia infection of murine plasmacytoid dendritic cells induces type I IFN production via the TLR9/MyD88/IRF5/IRF7-dependent pathway (Dai et al., 2011). Myxoma virus E3 ortholog M029 retains the dsRNA-binding domain of E3 but lacks the Z-DNA binding domain of E3. It was found that the Z-DNA-binding domain of E3 (but probably not Z-DNA-binding activity per se) plays an important role in inhibiting poxviral sensing in murine and human pDCs (Dai et al., 2011; Cao et al., 2012).

Deletion of E3L sensitizes vaccinia virus replication to IFN inhibition in permissive RK13 cells and results in a host range phenotype, whereby ΔE3L cannot replicate in HeLa or BSC40 cells (Chang et al., 1995). The C-terminal dsRNA-binding domain of E3 is responsible for the host range effects, whereas E3LΔ83N virus with deletion of the N-terminal Z-DNA-binding domain is replication competent in HeLa and BSC40 cells (Brandt et al., 2001).

Vaccinia virus (Western Reserve strain; WR) with deletion of thymidine kinase is highly attenuated in non-dividing cells but is replicative in transformed cells (Buller et al., 1988). TK-deleted vaccinia virus selectively replicates in tumor cells in vivo (Puhlmann et al., 2000). Thorne et al. showed that compared with other vaccinia strains, WR strain has the highest burst ratio in tumor cell lines relative to normal cells (Thorne et al., 2007).

### IX. Vaccinia Virus E5 is a Dominant Inhibitor of the Cytosolic DNA Sensor cGAS

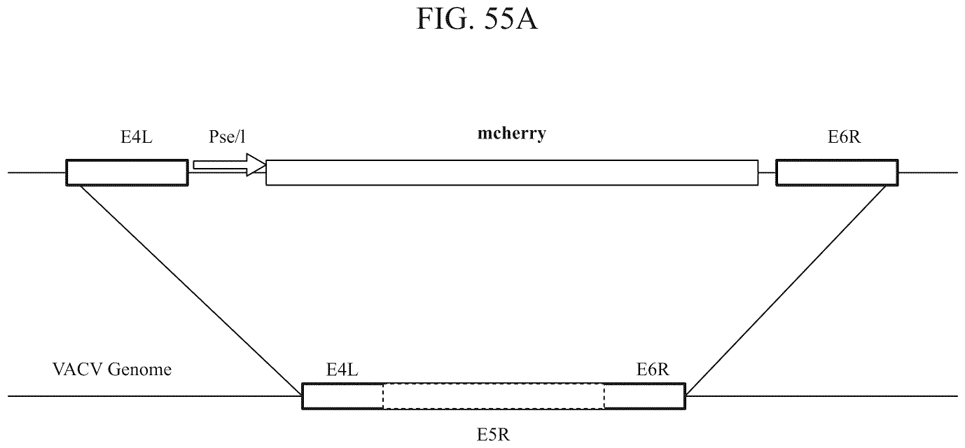

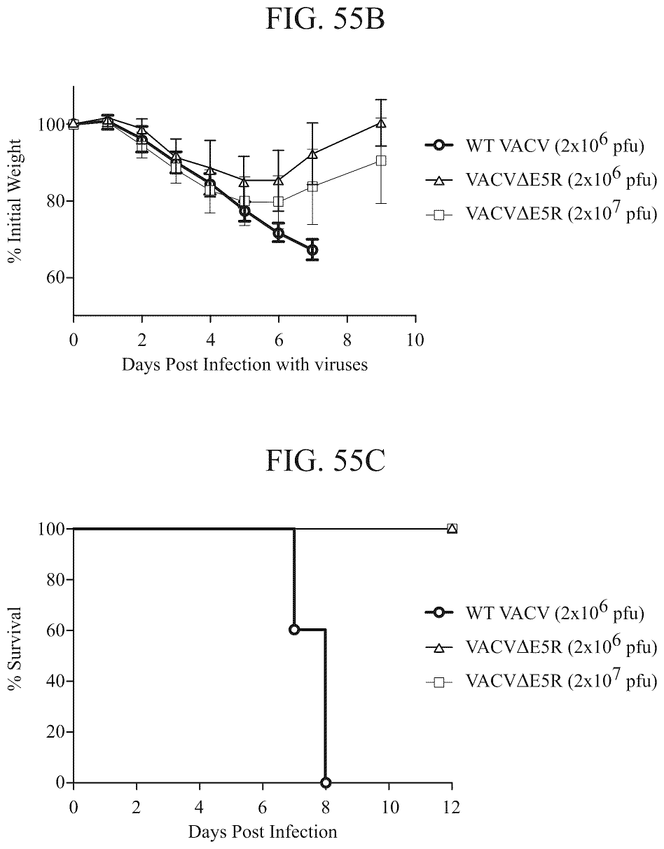

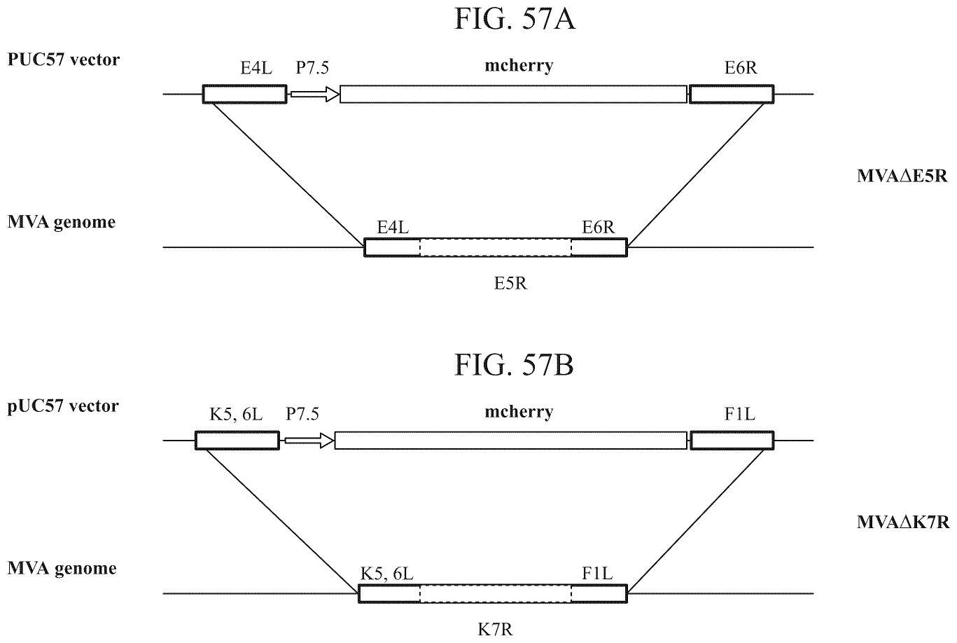

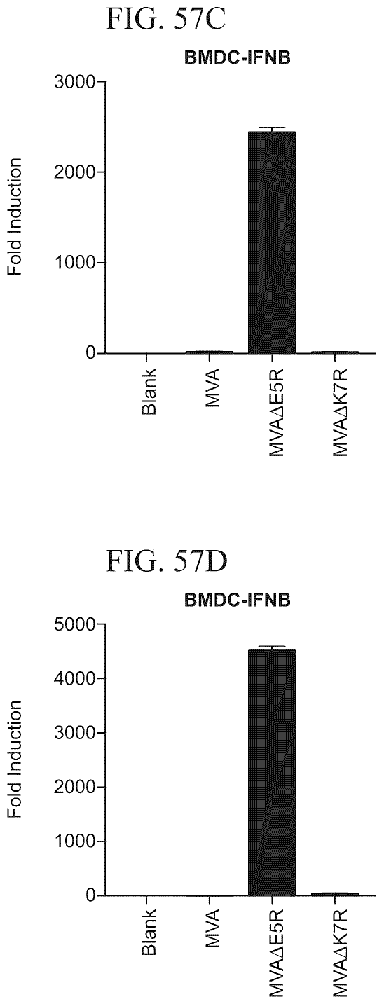

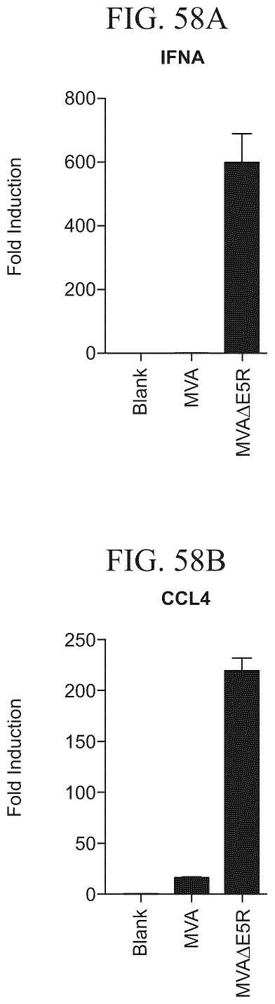

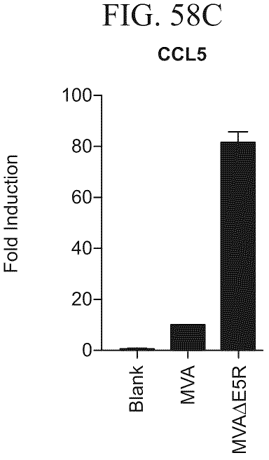

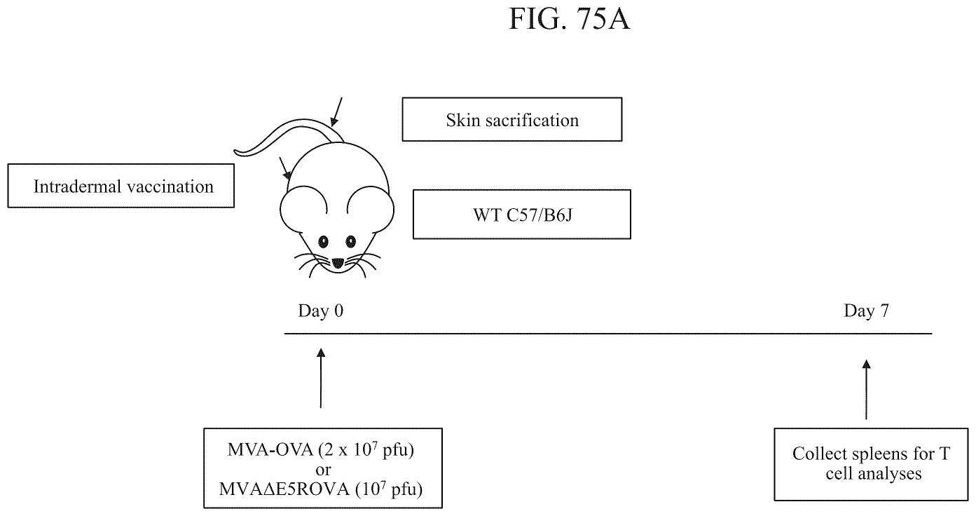

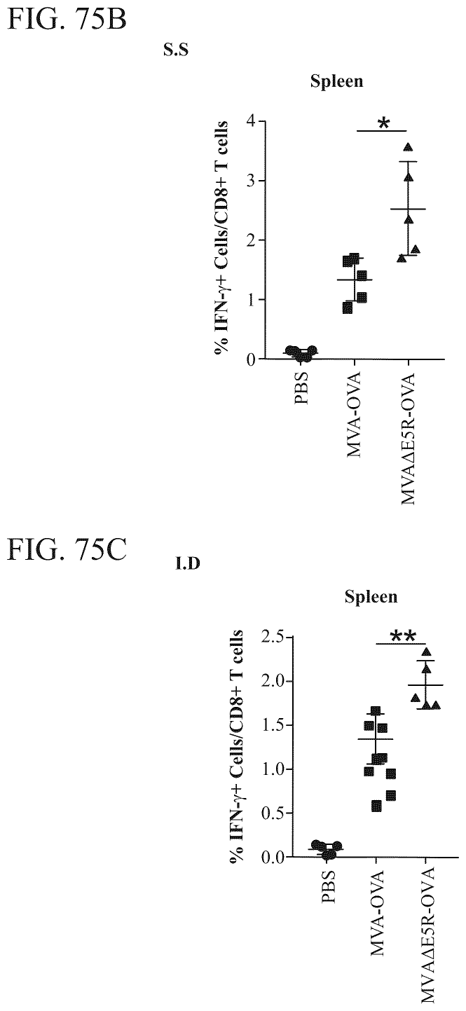

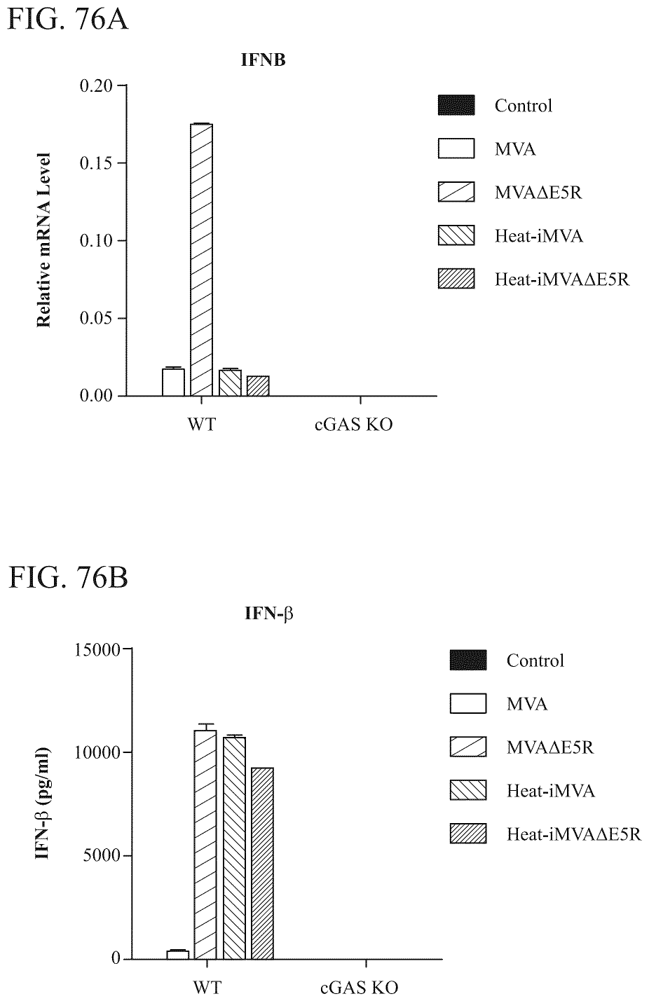

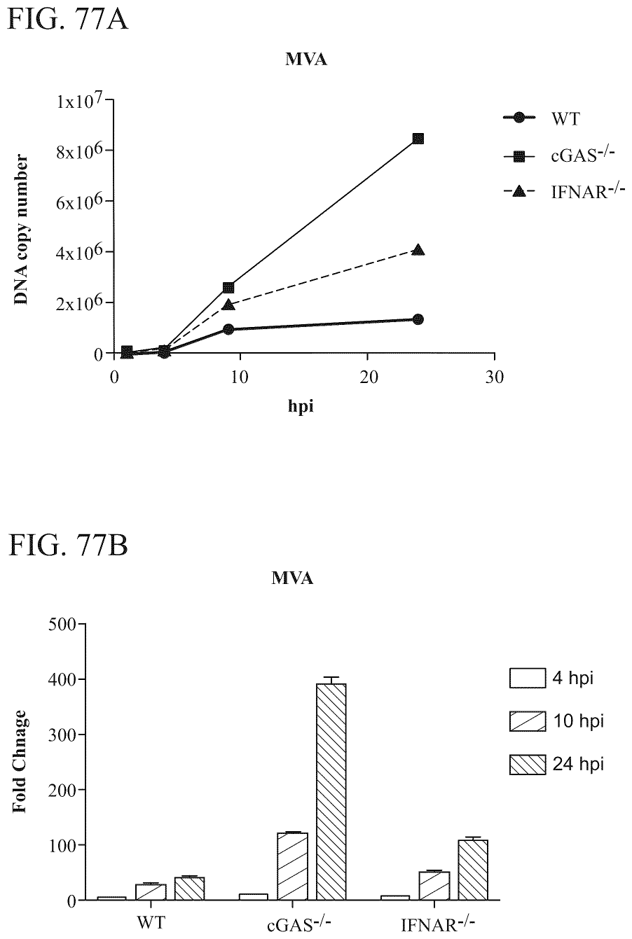

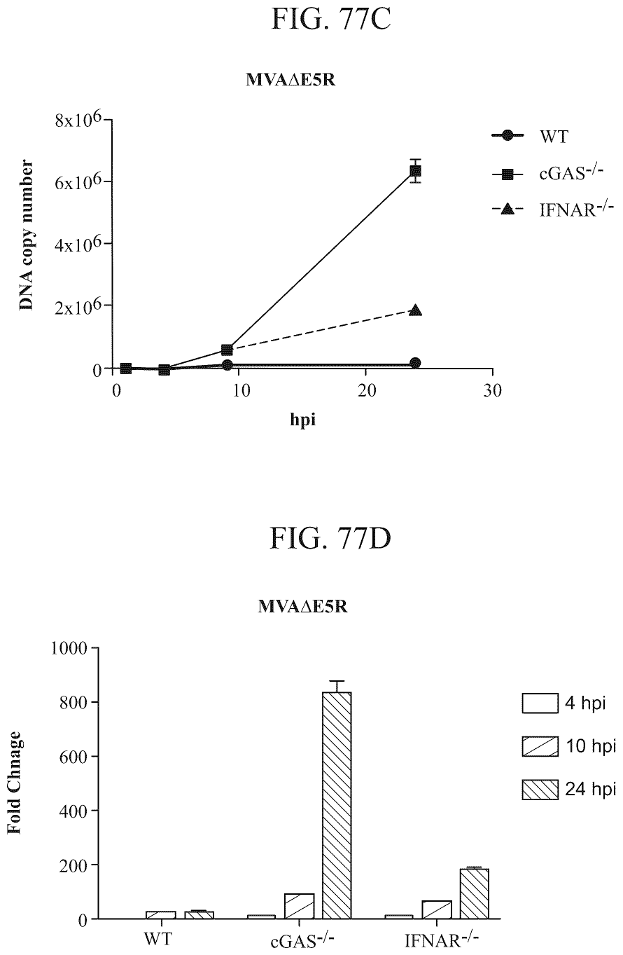

The cytosolic DNA sensor cGAS plays an important role in detecting viral nucleic acid, which leads to type I IFN production. It has been shown that infection of conventional dendritic cells with modified vaccinia virus Ankara (MVA), a highly attenuated vaccinia strain, induces IFN production via a cGAS/STING-dependent mechanism. However, MVA infection triggers cGAS degradation. Vaccinia virus (VACV) is a cytoplasmic DNA virus, which encodes more than 200 genes. As described in the experimental examples section, seventy vaccinia viral early genes were screened for inhibition of cGAS/STING pathway in HEK293 T cells using a dual luciferase system. It was found that vaccinia E5R is a dominant inhibitor of the cGAS and is the key protein mediating cGAS degradation. MVAΔE5R induces much higher levels of type I IFN than MVA in multiple cell types, including bone marrow derived dendritic cells (BMDC), bone marrow-derived macrophages (BMDM), and skin primary fibroblasts (FIGS. 57C and 57D; 76A and 76B). MVAΔE5R-mediated type I IFN production is dependent on cGAS (FIGS. 58A-58C). Furthermore, MVAΔE5R gains replication capability in cGAS^{-/-} skin fibroblasts (FIGS. 77C and 77D). As a vaccine vector, skin scarification or intradermal vaccination with MVAΔE5R-OVA leads to much higher OVA-specific CD8⁺ T cell responses than MVA-OVA in vivo (FIGS. 75B and 75C). Intratumoral injection of MVAΔE5R leads to stronger anti-tumor immune responses and better survival compared with MVA (FIGS. 91A-91C). Finally, in an intranasal infection model, VACVΔE5R is at least 1000-fold attenuated compared with WT VACV (FIG. 55B). Taken together, these results provide strong evidence that E5 is a key viral virulence factor targeting the cytosolic DNA sensor cGAS and thereby inhibits type I IFN production. The inventors of the present technology are the first to describe the role of E5R in immune evasion.

An illustrative full-length vaccinia virus E5R host range protein, given by GenBank Accession No. AAB59825.1 (SEQ ID NO: 20) is provided below.

MLILTKVNIYMLIIVLWLYGYNFIISESQCPMINDDSFTLKRKYQIDSA
ESTIKMDKKRTKFQNRAKMVKEINQTIRAAQTHYETLKLGYIKFKRMIR
TTTLEDIAPSIPNNQKTYKLESDISAIGKASRNPSKMVYALLLYMFPNL
FGDDHRFIRYRMHPMSKIKHKIFSPFKLNLIRILVEERFYNNECRSNKW
RIIGTQVDKMLIAESDKYTIDARYNLKPMYRIKGKSEEDTLFIKQMVEQ
CVTSQELVEKVLKILFRDLFKSGEYKAYRYDDDVENGFIGLDTLKLNIV
HDIVEPCMPVRRPVAKILCKEMVNKYFENPLHIIGKNLQECIDEVSE

The myxoma ortholog of vaccinia virus E5R is M31R. An illustrative full-length myxoma virus M31R protein, given by GenBank Accession No. AAF14919.1 (SEQ ID NO: 21), is provided below.

MEGDYLIRPG EKQASYACRL LGILTKHSTY PPEEYEPLVR
SIMSMYNTLI KDDVIWFREI APYLYEYTMY KQNARNPSFY
ISTNVVNLTT CRVSKSSAKS AKYRAKSKQM KMRRVADGVP
FEEKLKRDEA IRQKNKKDYF EIKKLYMRLK KFVRGKKSAD
DNMLCNKVRM IYGHINEIER VAVNEYSMAK SLLHYVEPNL
FNDDKHHLFY RCTKMDGLGV LPSKKLNLIR VILENKFKIS
KRKWTMLKKY IDTVCATGKL RVRLGTYPYY KLKSLNALVA
SYQGDSVDEL KTLVLSSFSL VDLTEKLIKT TFPEVVKSGE
GHNYRCYPDG THQGLDPERV IDMCYKARVA TDSESVVDVH
NAIVETVNRF LIRSEKKVGD NIDECIVMAK TIN

### X. Engineered Poxvirus Strains of the Present Technology MVAΔC7L

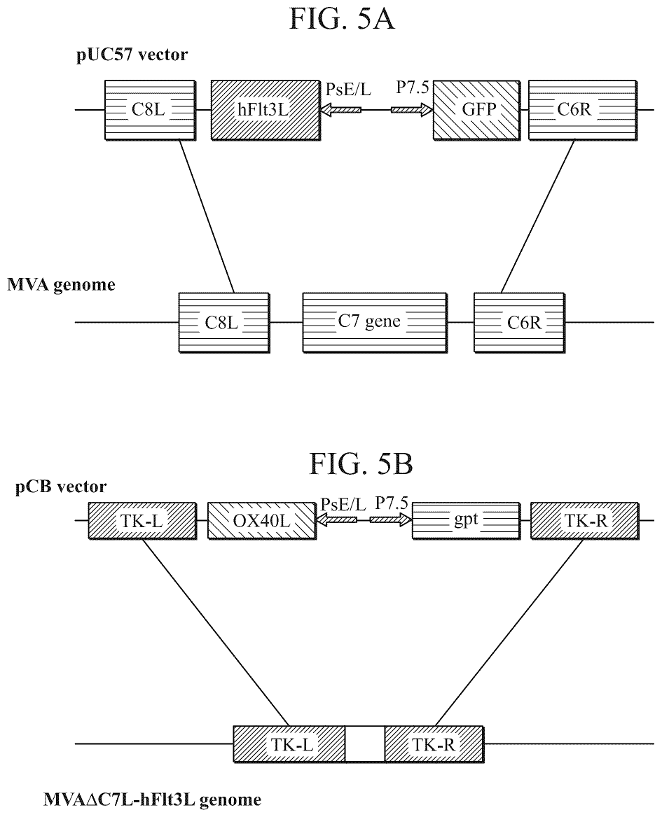

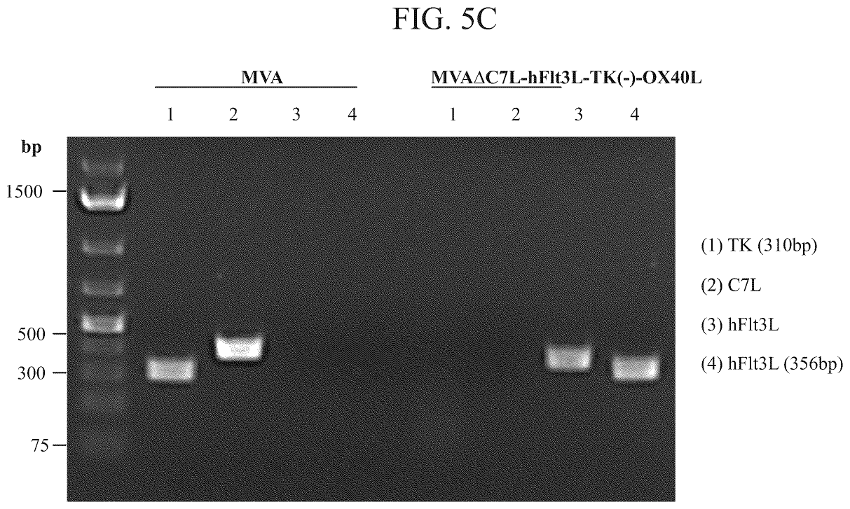

The disclosure of the present technology relates to a C7L mutant modified vaccinia Ankara (MVA) virus (i.e., MVAΔC7L; MVA virus comprising a C7L deletion; MVA genetically engineered to comprise a mutant C7L gene), or immunogenic compositions comprising the virus, in which the virus is engineered to express one or more specific genes of interest (SG), such as OX40L (MVAΔC7L-OX40L), and their use as a cancer immunotherapeutic. In some embodiments, the C7 gene of the MVA virus, through homologous recombination techniques, is engineered to contain a disruption comprising a heterologous nucleic acid sequence comprising one or more expression cassettes, which result in a C7 knockout such that the C7 gene is not expressed, expressed at levels so low as to have no effect, or the expressed protein is non-functional (e.g., is a null-mutation). In some embodiments, the ΔC7L mutant includes a heterologous nucleic acid sequence in place of all or a majority of the C7L gene sequence. For example, in some embodiments, the nucleic acid sequence corresponding to the position of C7 in the MVA genome (e.g., position 18,407 to 18,859 of SEQ ID NO: 1) is replaced with a heterologous nucleic acid sequence comprising an open reading frame that encodes a specific gene of interest (SG), such as human OX40L, resulting in MVAΔC7L-OX40L. In some embodiments, the expression cassette comprises a single open reading frame that encodes hFlt3L, resulting in MVAΔC7L-hFlt3L. (See, e.g., FIG. 5A).

Additionally or alternatively, in some embodiments, MVAΔC7L is engineered to express both OX40L and hFlt3L. In some embodiments, the thymidine kinase (TK) gene of the MVA virus (e.g., position 75,560 to 76,093 of SEQ ID NO: 1), through homologous recombination, is engineered to contain a disruption comprising a heterologous nucleic acid sequence comprising one or more expression cassettes, which results in a TK gene knockout such that the TK gene is not expressed, expressed at levels so low as to have no effect, or the expressed protein is non-functional (e.g., is a null-mutation). The resulting MVAΔC7L-TK(−) virus is further engineered to comprise one or more expression cassettes that are flanked by a partial sequence of the TK gene (TK-L and TK-R) on either side (see, e.g., FIG. 5B). In some embodiments, the expression cassette comprises a single open reading frame that encodes a specific gene of interest (SG), such as OX40L or hFlt3L using the vaccinia viral synthetic early and late promoter (PsE/L), resulting in MVAΔC7L-TK(−)-OX40L. In some embodiments, the recombinant virus is further modified at the C7 locus, through homologous recombination techniques, to contain a disruption comprising a heterologous nucleic acid sequence comprising one or more expression cassettes, which result in a C7 knockout such that the C7 gene is not expressed, expressed at levels so low as to have no effect, or the expressed protein is non-functional (e.g., is a null-mutation). In some embodiments, the expression cassette comprises a single open reading frame that encodes a specific gene of interest (SG), such as hFlt3L, resulting in MVAΔC7L-hFlt3L-TK(−)-OX40L. In some embodiments, the expression cassette encoding OX40L is inserted into the C7 locus while the expression cassette encoding hFlt3L is inserted into the TK locus.

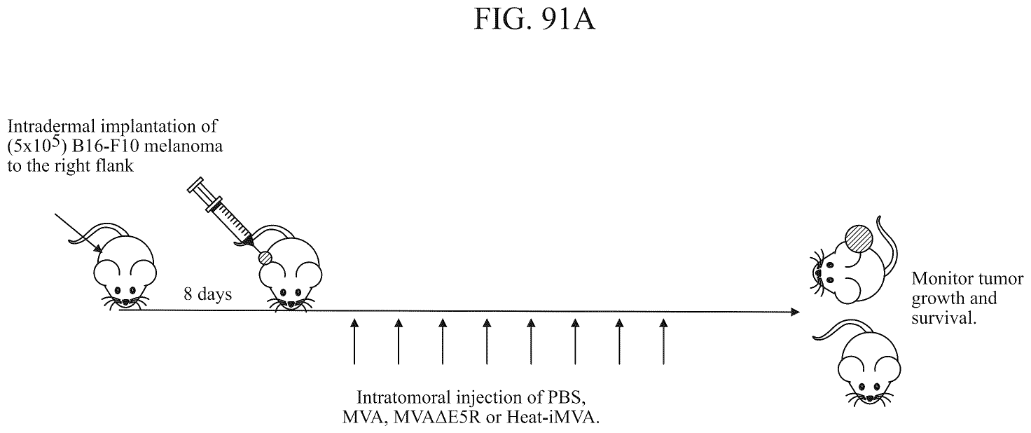

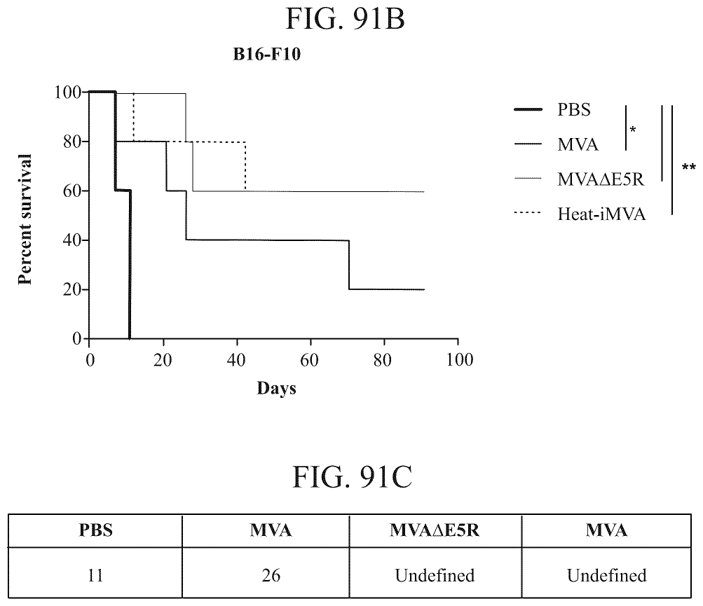

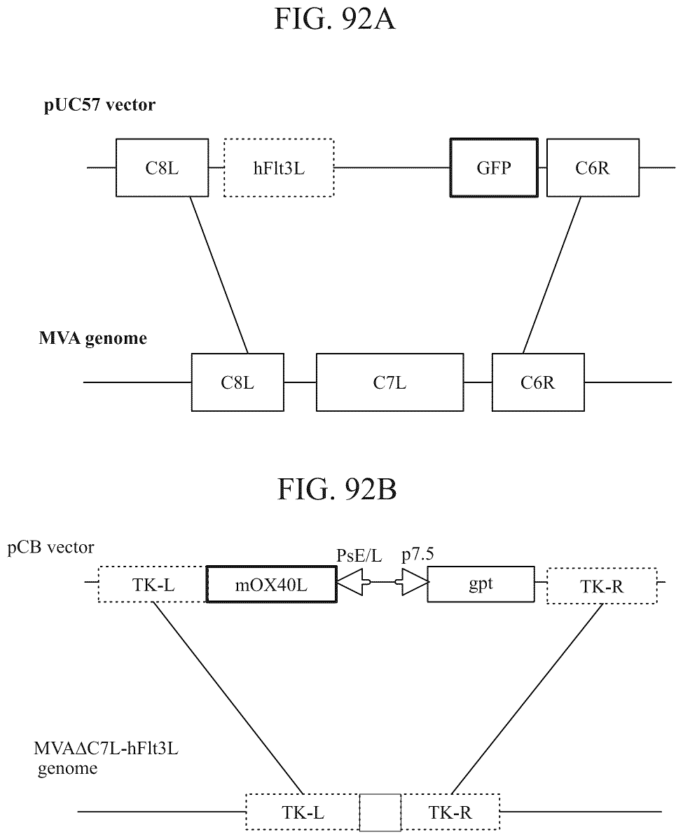

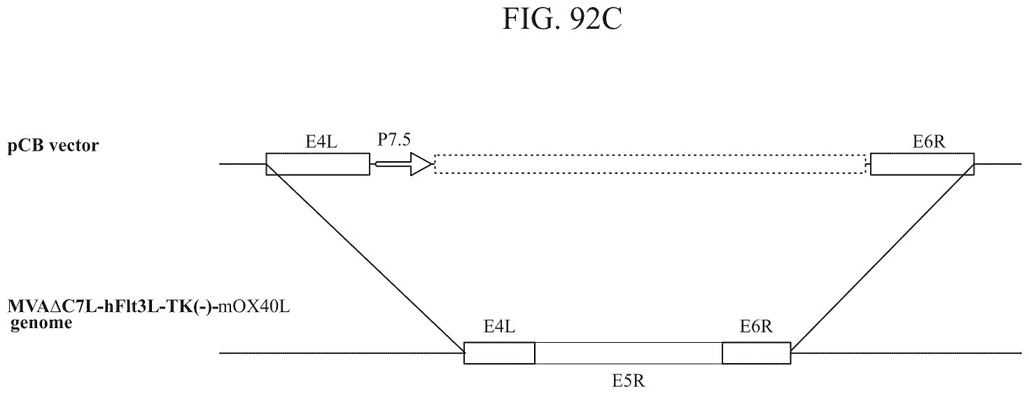

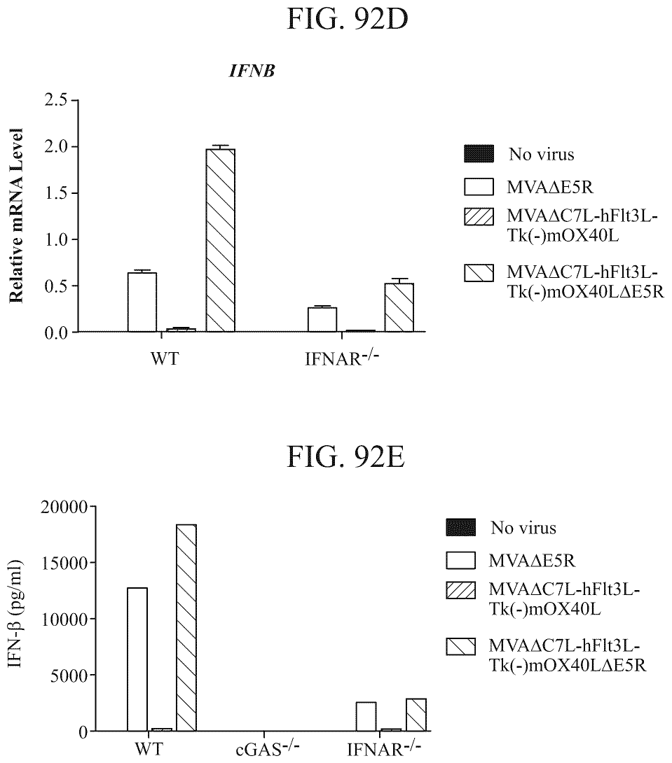

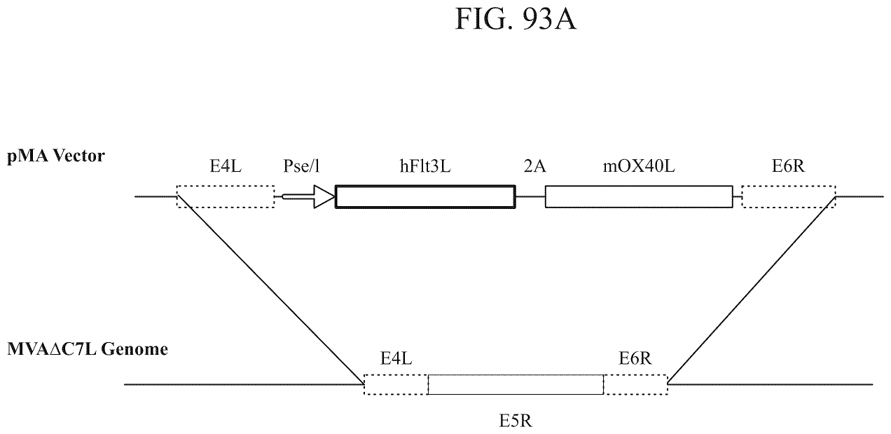

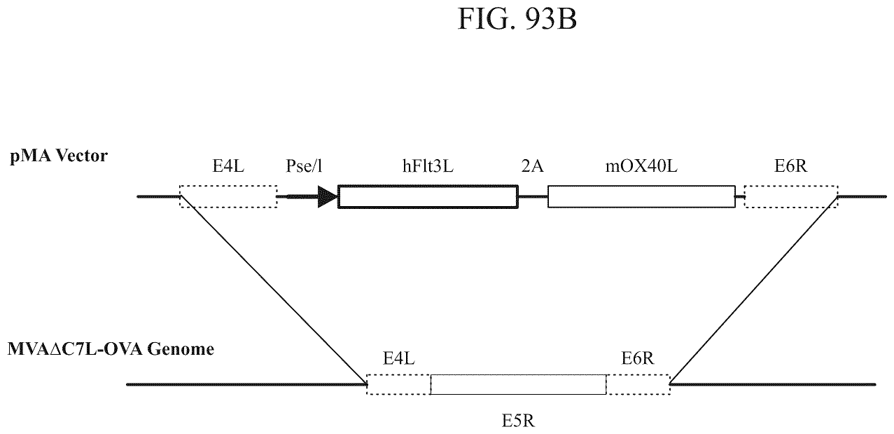

Additionally or alternatively, in some embodiments, the heterologous nucleic acid sequence comprises an expression cassette comprising two or more open reading frames encoding two or more specific genes of interest, separated by a nucleotide sequence that encodes, in the 5′ to 3′ direction, a protease cleavage site (e.g., a furin cleavage site) and a 2A peptide (Pep2A) sequence. For example, in some embodiments, MVAΔC7L encompasses a recombinant MVA virus in which all or a majority of the E5R gene sequence is replaced by a first specific gene of interest (e.g., hFtl3L) and a second specific gene of interest (e.g., OX40L), wherein the coding sequences of the first and second specific genes of interest are separated by a cassette including a furin cleavage site followed by a 2A peptide (Pep2A) sequence, thereby forming a recombinant virus such as MVAΔC7LΔE5R-hFlt3L-OX40L (see, e.g., FIG. 93A). As another example, in some embodiments, the present technology provides a recombinant MVAΔC7L-hFlt3L-TK(−)-OX40LΔE5R virus that is formed by making three separate modifications. The first modification involves replacing the C7L gene, through homologous recombination at the C8L and C6R loci, with a heterologous nucleic acid sequence comprising an expression cassette comprising an open reading frame encoding hFlt3L, thereby forming MVAΔC7L-hFlt3L. For the second modification, through homologous recombination at the TK locus, the TK gene is replaced with a heterologous nucleic acid sequence comprising an expression cassette comprising an open reading frame encoding OX40L, thereby forming MVAΔC7L-hFlt3L-TK(−)-OX40L. For the third modification, through homologous recombination at the E4L and E6R loci, the E5R gene is replaced with a heterologous nucleic acid sequence comprising an expression cassette comprising an open reading frame encoding a selectable marker, such as mCherry, thereby forming MVAΔC7L-hFlt3L-TK(−)-OX40LΔE5R (see, e.g., FIG. 92A).

In some embodiments, the recombinant MVAΔC7L-OX40L viruses described above are modified to express at least one additional heterologous gene, such as any one or more of hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or include any one or more of the following deletions: E3L (ΔE3L); E3LΔ83N; B2R (ΔB2R), B19R (B18R; ΔWR200); E5R; K7R; C12L (IL18BP); B8R; B14R; N1L; C11R; K1L; M1L, N2L, and/or WR199.

Although in certain embodiments described above, the transgene may be inserted into the TK locus, splitting the TK gene and obliterating it, other suitable integration loci can be selected. For example, MVA encodes several immune modulatory genes, including but not limited to C11, K3, F1, F2, F4, F6, F8, F9, F11, F14.5, J2, A46, E3L, B18R (WR200), E5R, K7R, C12L, B8R, B14R, N1L, C11R, K1L, C16, M1L, N2L, and WR199. Accordingly, in some embodiments, these genes can be deleted to potentially enhance immune activating properties of the virus and allow insertion of transgenes. For example, in some embodiments, the present technology provides an MVAΔC7L-hFlt3L-TK(−)-OX40L virus expressing transgenes IL-2, IL-12, IL-18, IL-15, and/or IL-21 from within the E5R gene locus. In other embodiments, no further heterologous genes are added other than those provided in the name of the virus herein (e.g. OX40L or OX40L and hFlt3L), and/or no further viral genes other than C7L or C7L and TK are disrupted or deleted.

In some embodiments, the heterologous nucleotide sequence further comprises an additional expression cassette comprising an open reading frame that encodes a selectable marker operably linked to a promoter that is capable of directing expression of the selectable marker (FIGS. 5A-5B). In some embodiments, the selectable marker is a xanthine-guanine phosphoribosyl transferase (gpt) gene. In some embodiments, the selectable marker is a green fluorescent protein (GFP) gene. In some embodiments, the selectable marker is an mCherry gene encoding a red fluorescent protein.

Non-limiting examples of OX40L expression construct open reading frames according to the present technology are shown in SEQ ID NOs: 3 and 5 (Table 1). A non-limiting example of an hFlt3L expression construct according to the present technology is shown in SEQ ID NO: 4 (Table 1).

TABLE 1
Exemplary nucleotide sequences for the open reading frames of
the recombinant MVA constructs of the present technology.

```
pCB-mOX40L-gpt vector nucleic acid sequence
(SEQ ID NO: 3)
CGCGCGTAATACGACTCACTATAGGGCGAATTGGAGCTCTTTTTATCTGCGCGGTTAAC
CGCCTTTTTATCCATCAGGTGATCTGTTTTTATTGTGGAGTCTAGATTATCACAGTGGG
ACTTGGTTCACTGTACTGTGATAACTGCCCTCAGGAGCACAGTAACCTGGTGTGAGTTG
GACAACGATAAGTTCTCCATCGTTGATTTGCAAATGCTCGCACAAGGTATCAGGAGCG
TTTACGGTCAAGTACACCTTATCCTTGAAGGCCAGTGAAGCGACTACAGTAAAGACGA
TTCTTCTGCCGTCGTTCAACATAGGGATTGAGATAGGGTTGTGATCCTCCCTGAAGTGG
AGGTCAATTTTGACCTCCTGGAAAAAAGATCCTTTCAAGTAGATGATGTACAGTCCGT
CACACTTAATCACTACGGAGTTATTCTGGACTTCCATCGTCTGATACTCGTTCTTGTAA
GAAGAAATAAACAATTGACCATCTTCACAGCGGGTCACAGCCCCCCGAAGGCGTTGTA
TGGGGGGGTCTTTTGCAGGTGAAGAGCTAAGTTGCAAACACACATATATGAAGCACAG
CAACATACCGGCCCCCTTGATGCCGCTTACAACGAGCCTAAGAGTCTTCTTCCATTTAA
AGCGAGGGCGAGACCCGTTTTCGAGGTTCTCGTCCAGAGGCTGGACCCCCTCGCCCTC
CATGGTGGTGGCCTAGAATTCGATATCAAGCTCAGGCCTAGATCTGTCGACTTCGAGC
TTATTTATATTCCAAAAAAAAAAAATAAAATTTCAATTTTTAAGCTTTCACTAATTCCA
AACCCACCCGCTTTTTATAGTAAGTTTTTCACCCATAAATAATAAATACAATAATTAAT
TTCTCGTAAAAGTAGAAAATATATTCTAATTTATTGCACGGTAAGGAAGTAGATCATA
ACTCGAGGAATTGGGGATCTCTATAATCTCGCGCAACCTATTTTCCCCTCGAACACTTT
TTAAGCCGTAGATAAACAGGCTGGGACACTTCACATGAGCGAAAAATACATCGTCACC
TGGGACATGTTGCAGATCCATGCACGTAAACTCGCAAGCCGACTGATGCCTTCTGAAC
AATGGAAAGGCATTATTGCCGTAAGCCGTGGCGGTCTGGTACCGGGTGCGTTACTGGC
GCGTGAACTGGGTATTCGTCATGTCGATACCGTTTGTATTTCCAGCTACGATCACGACA
ACCAGCGCGAGCTTAAAGTGCTGAAACGCGCAGAAGGCGATGGCGAAGGCTTCATCG
TTATTGATGACCTGGTGGATACCGGTGGTACTGCGGTTGCGATTCGTGAAATGTATCCA
AAAGCGCACTTTGTCACCATCTTCGCAAAACCGGCTGGTCGTCCGCTGGTTGATGACTA
TGTTGTTGATATCCCGCAAGATACCTGGATTGAACAGCCGTGGGATATGGGCGTCGTA
TTCGTCCCGCCAATCTCCGGTCGCTAATCTTTTCAACGCCTGGCACTGCCGGGCGTTGT
TCTTTTTAACTTCAGGCGGGTTACAATAGTTTCCAGTAAGTATTCTGGAGGCTGCATCC
ATGACACAGGCAAACCTGCGGATCCCAGCTTTTGTTCCCTTTAGTGAGGGTTAATTGCG
CGCAGTTATAGTAGCCGCACTCGATGGGACATTTCAACGTAAACCGTTTAATAATATTT
TGAATCTTATTCCATTATCTGAAATGGTGGTAAAACTAACTGCTGTGTGTATGAAATGC
TTTAAGGAGGCTTCCTTTTCTAAACGATTGGGTGAGGAAACCGAGATAGAAATAATAG
GAGGTAATGATATGTATCAATCGGTGTGTAGAAAGTGTTACATCGACTCATAATATTA
TATTTTTTATCTAAAAAACTAAAAATAAACATTGATTAAATTTTAATATAATACTTAAA
AATGGATGTTGTGTCGTTAGATAAACCGTTTATGTATTTTGAGGAAATTGATAATGAGT
TAGATTACGAACCAGAAAGTGCAAATGAGGTCGCAAAAAAACTGCCGTATCAAGGAC
AGTTAAAACTATTACTAGGAGAATTATTTTTTCTTAGTAAGTTACAGCGACACGGTATA
TTAGATGGTGCCACCGTAGTGTATATAGGATCTGCTCCCGGTACACATATACGTTATTT
GAGAGATCATTTCTATAATTTAGGAGTGATCATCAAATGGATGCTAATTGACGGCCGC
CATCATGATCCTATTTTAAATGGATTGCGTGATGTGACTCTAGTGACTCGGTTCGTTGA
TGAGGAATATCTACGATCCATCAAAAAACAACTGCATCCTTCTAAGATTATTTTAATTT
CTGATGTGAGATCCAAACGAGGAGGAAATGAACCTAGTACGGCGGATTTACTAAGTA
ATTACGCTCTACAAAATGTCATGATTAGTATTTTAAACCCCGTGGCGTCTAGTCTTAAA
TGGAGATGCCCGTTTCCAGATCAATGGATCAAGGACTTTTATATCCCACACGGTAATA
AAATGTTACAACCTTTTGCTCCTTCATATTCAGCTGAAATGAGATTATTAAGTATTTAT
ACCGGTGAGAACATGAGACTGACTCGGGCCGCGTTGCTGGCGTTTTTCCATAGGCTCC
GCCCCCCTGACGAGCATCACAAAAATCGACGCTCAAGTCAGAGGTGGCGAAACCCGA
CAGGACTATAAAGATACCAGGCGTTTCCCCCTGGAAGCTCCCTCGTGCGCTCTCCTGTT
CCGACCCTGCCGCTTACCGGATACCTGTCCGCCTTTCTCCCTTCGGGAAGCGTGGCGCT
TTCTCAATGCTCACGCTGTAGGTATCTCAGTTCGGTGTAGGTCGTTCGCTCCAAGCTGG
GCTGTGTGCACGAACCCCCCGTTCAGCCCGACCGCTGCGCCTTATCCGGTAACTATCGT
CTTGAGTCCAACCCGGTAAGACACGACTTATCGCCACTGGCAGCAGCCACTGGTAACA
GGATTAGCAGAGCGAGGTATGTAGGCGGTGCTACAGAGTTCTTGAAGTGGTGGCCTAA
CTACGGCTACACTAGAAGGACAGTATTTGGTATCTGCGCTCTGCTGAAGCCAGTTACCT
TCGGAAAAAGAGTTGGTAGCTCTTGATCCGGCAAACAAACCACCGCTGGTAGCGGTGG
TTTTTTTGTTTGCAAGCAGCAGATTACGCGCAGAAAAAAAGGATCTCAAGAAGATCCT
TTGATCTTTTCTACGGGGTCTGACGCTCAGTGGAACGAAAACTCACGTTAAGGGATTTT
GGTCATGAGATTATCAAAAAGGATCTTCACCTAGATCCTTTTAAATTAAAAATGAAGT
TTTAAATCAATCTAAAGTATATATGAGTAAACTTGGTCTGACAGTTACCAATGCTTAAT
CAGTGAGGCACCTATCTCAGCGATCTGTCTATTTCGTTCATCCATAGTTGCCTGACTCC
CCGTCGTGTAGATAACTACGATACGGGAGGGCTTACCATCTGGCCCCAGTGCTGCAAT
GATACCGCGAGACCCACGCTCACCGGCTCCAGATTTATCAGCAATAAACCAGCCAGCC
GGAAGGGCCGAGCGCAGAAGTGGTCCTGCAACTTTATCCGCCTCCATCCAGTCTATTA
ATTGTTGCCGGGAAGCTAGAGTAAGTAGTTCGCCAGTTAATAGTTTGCGCAACGTTGTT
GCCATTGCTGCAGGCATCGTGGTGTCACGCTCGTCGTTTGGTATGGCTTCATTCAGCTC
CGGTTCCCAACGATCAAGGCGAGTTACATGATCCCCCATGTTGTGCAAAAAAGCGGTT
AGCTCCTTCGGTCCTCCGATCGTTGTCAGAAGTAAGTTGGCCGCAGTGTTATCACTCAT
GGTTATGGCAGCACTGCATAATTCTCTTACTGTCATGCCATCCGTAAGATGCTTTTCTG
TGACTGGTGAGTACTCAACCAAGTCATTCTGAGAATAGTGTATGCGGCGACCGAGTTG
CTCTTGCCCGGCGTCAACACGGGATAATACCGCGCCACATAGCAGAACTTTAAAAGTG
CTCATCATTGGAAAACGTTCTTCGGGGCGAAAACTCTCAAGGATCTTACCGCTGTTGA
GATCCAGTTCGATGTAACCCACTCGTGCACCCAACTGATCTTCAGCATCTTTTACTTTC
ACCAGCGTTTCTGGGTGAGCAAAAACAGGAAGGCAAAATGCCGCAAAAAAGGGAATA
AGGGCGACACGGAAATGTTGAATACTCATACTCTTCCTTTTTCAATATTATTGAAGCAT
TTATCAGGGTTATTGTCTCATGAGCGGATACATATTTGAATGTATTTAGAAAAATAAAC
AAATAGGGGTTCCGCGCACATTTCCCCGAAAAGTGCCACCTGACGTCTAAGAAACCAT
TATTATCATGACATTAACCTATAAAAATAGGCGTATCACGAGGCCCTTTCGTCTTCGAA
TAAATACCTGTGACGGAAGATCACTTCGCAGAATAAATAAATCCTGGTGTCCCTGTTG
ATACCGGGAAGCCCTGGGCCAACTTTTGGCGAAAATGAGACGTTGATCGGCACGTAAG
AGGTTCCAACTTTCACCATAATGAAATAAGATCACTACCGGGCGTATTTTTTGAGTTAT
CGAGATTTTCAGGAGCTAAGGAAGCTAAAATGGAGAAAAAAATCACTGGATATACCA
CCGTTGATATATCCCAATGGCATCGTAAAGAACATTTTGAGGCATTTCAGTCAGTTGCT
CAATGTACCTATAACCAGACCGTTCAGAGCTTTTGGGATCAATAAATGGATCACAACC
AGTATCTCTTAACGATGTTCTTCGCAGATGATGATTCATTTTTTAAGTATTTGGCTAGTC
AAGATGATGAATCTTCATTATCTGATATATTGCAAATCACTCAATATGTAGCTAGACTT
TCTGTTATTATTATTGATCCAATCAAAAAATAAATTAGAAGCCGTGGGTCATTGTTATG
AATCTCTTTCAGAGGAATACAGACAATTGACAAAATTCACAGACTTTCAAGATTTTAA
AAAACTGTTTAACAAGGTCCCTATTGACAGATGGAAGGGTCAAACTTAATAAAGGATA
TTTGTTCGACTTTGTGATTAGTTTGATGCGATTCAAAAAAGAATCCTCTCTAGCTACCA
CCGCAATAGATCCTGTTAGATACATAGATCCTCGTCGCAATATCGCATTTTCTAACGTG
ATGGATATATTAAAGTCGAATAAAGTGAACAATAATTAATTCTTTATTGTCATCATGAA
CGGCGGACATATTCAGTTGATAATCGGCCCCATGTTTTCAGGTAAAAGTACAGAATTA
ATTAGACGAGTTAGACGTTATCAAATAGCTCAATATAAATGCGTGACTATAAAATATT
CTAACGATAATAGATACGGAACGGGACTATGGACGCATGATAAGAATAATTTTGAAGC
ATTGGAAGCAACTAAACTATGTGATCTCTTGGAATCAATTACAGATTTCTCCGTGATAG
G
pUC57-delC7-hOX40L-mCherry vector nucleic acid sequence
(SEQ ID NO: 5)
      1 TCGCGCGTTT CGGTGATGAC GGTGAAAACC TCTGACACAT GCAGCTCCCG GAGACGGTCA
    61 CAGCTTGTCT GTAAGCGGAT GCCGGGAGCA GACAAGCCCG TCAGGGCGCG TCAGCGGGTG
  121 TTGGCGGGTG TCGGGGCTGG CTTAACTATG CGGCATCAGA GCAGATTGTA CTGAGAGTGC
  181 ACCATATGCG GTGTGAAATA CCGCACAGAT GCGTAAGGAG AAAATACCGC ATCAGGCGCC
  241 ATTCGCCATT CAGGCTGCGC AACTGTTGGG AAGGGCGATC GGTGCGGGCC TCTTCGCTAT
  301 TACGCCAGCT GGCGAAAGGG GGATGTGCTG CAAGGCGATT AAGTTGGGTA ACGCCAGGGT
  361 TTTCCCAGTC ACGACGTTGT AAAACGACGG CCAGTGAATT CGAGCTCGGT ACCTCGCGAA
  421 TGCATCTAGA TTCGCAGATG ATGATTCATT TTTTAAGTAT TTGGCTAGTC AAGATGATGA
  481 ATCTTCATTA TCTGATATAT TGCAAATCAC TCAATATCTA GACTTTCTGT TATTATTATT
  541 GATCCAATCA AAAAATAAAT TAGAAGCCGT GGGTCATTGT TATGAATCTC TTTCAGAGGA
  601 ATACAGACAA TTGACAAAAT TCACAGACTT TCAAGATTTT AAAAAACTGT TTAACAAGGT
  661 CCCTATTGTT ACAGATGGAA GGGTCAAACT TAATAAAGGA TATTTGTTCG ACTTTGTGAT
  721 TAGTTTGATG CGATTCAAAA AAGAATCCTC TCTAGCTACC ACCGCAATAG ATCCTATTAG
  781 ATACATAGAT CCTCGTCGCG ATATCGCATT TTCTAACGTG ATGGATATAT TAAAGTCGAA
  841 TAAAGTGAAC AATAATTAAT TCTTTATTGT CATCAGGCCT AGAAAAAGCT AGTTCACTAA
  901 TTCCAAACCC ACCCGCTTTT TATAGTAAGT TTTTCACCCA TAAATAATAA ATACAATAAT
  961 TAATTTCTCG TAAAAGTAGA AAATATATTC TAATTTATTG CACGGTAAGG AAGTAGATCA
1021 TAACTCGACA TGGTGAGCAA GGGCGAGGAG GATAACATGG CCATCATCAA GGAGTTCATG
1081 CGCTTCAAGG TGCACATGGA GGGCTCCGTG AACGGCCACG AGTTCGAGAT CGAGGGCGAG
1141 GGCGAGGGCC GCCCCTACGA GGGCACCCAG ACCGCCAAGC TGAAGGTGAC CAAGGGTGGC
1201 CCCCTGCCCT TCGCCTGGGA CATCCTGTCC CCTCAGTTCA TGTACGGCTC CAAGGCCTAC
1261 GTGAAGCACC CCGCCGACAT CCCCGACTAC TTGAAGCTGT CCTTCCCCGA GGGCTTCAAG
1321 TGGGAGCGCG TGATGAACTT CGAGGACGGC GGCGTGGTGA CCGTGACCCA GGACTCCTCC
1381 CTGCAGGACG GCGAGTTCAT CTACAAGGTG AAGCTGCGCG GCACCAACTT CCCCTCCGAC
1441 GGCCCCGTAA TGCAGAAGAA GACCATGGGC TGGGAGGCCT CCTCCGAGCG GATGTACCCC
1501 GAGGACGGCG CCCTGAAGGG CGAGATCAAG CAGAGGCTGA AGCTGAAGGA CGGCGGCCAC
1561 TACGACGCTG AGGTCAAGAC CACCTACAAG GCCAAGAAGC CCGTGCAGCT GCCCGGCGCC
1621 TACAACGTCA ACATCAAGTT GGACATCACC TCCCACAACG AGGACTACAC CATCGTGGAA
1681 CAGTACGAAC GCGCCGAGGG CCGCCACTCC ACCGGCGGCA TGGACGAGCT GATCACGAAT
1741 TCTAGCTCAA AGGACACAGA ATTCACCAGG ATTTTGATGG ATAAGAATCA GTTCTCCGCC
1801 ATTCACATGG AAGTCATCCA GGGAGGTATT GTCAGTGGTC ACATTCAAGT AGACTTTGTC
1861 TTTGTAAGTC AGAGAGGCCA CCATCAAGGA GTTGACAGAC CTGACCTTCT TCAGTTGGAA
1921 GAGGGGCTCC TCATCCTTCT GGTAATGAAG GCTAATGTTG ACTTCCTGGG AGAAGTAGCC
1981 CTTCAGGGAG ATGAGATAAA ACCCATCACA GTTGATGATG ACTGAGTTGT TCTGCACCTT
2041 CATGATTTCA TCCTCCTTTT GGGAAGTGAG GATGAAACCT TTCTCCTTCT TATATTCGGT
2101 AAATTGTACT TTGATACTTT GAATTCGAGG ATACCGATGT GATACCTGAA GAGCAGAGAA
2161 GTGCAGGCAG ATGTAGGTGA AGCACAGGAG CAGCCCCAGT CCCTGAATTA CAGAGGCCAC
2221 CAGCAATAGC TTGTTCCTCT CGAATCTTGG CCTGGCTGCA TTTCCCACAT TCTCTTCCAG
2281 GGGTTGGACC CTTTCCATGA ATTCGTCGAC TTCGAGCTTA TTTATATTCC AAAAAAAAAA
2341 AATAAAATTT CAATTTTTAA GCTTTATTAT ATTTTTTATC TAAAAAACTA AAAATAAACA
2401 TTGATTAAAT TTTAATATAA TACTTAAAAA TGGATGTTGT GTCGTTAGAT AAACCGTTTA
2461 TGTATTTTGA GGAAATTGAT AATGAGTTAG ATTACGAACC AGAAAGTGCA AATGAGGTCG
2521 CAAAAAAACT GCCGTATCAA GGACAGTTAA AACTATTACT AGGAGAATTA TTTTTTCTTA
2581 GTAAGTTACA GCGACACGGT ATATTAGATG GTGCCACCGT AGTGTATATA GGATCTGCTC
2641 CCGGTACACA TATACGTTAT TTGAGAGATC ATTTCTATAA TTTAGGAGTG ATCATCAAAT
2701 GGATGCTAAT TGACGGCCGC CATCATGATC CTATTTTAAA TGGATTGCGT GATGTGACTC
2761 TAGTGACTCG GTTCGTTGAT GAGGAATATC TACGATCCAT CAAAAAACAT CGGATCCCGG
2821 GCCCGTCGAC TGCAGAGGCC TGCATGCAAG CTTGGCGTAA TCATGGTCAT AGCTGTTTCC
2881 TGTGTGAAAT TGTTATCCGC TCACAATTCC ACACAACATA CGAGCCGGAA GCATAAAGTG
2941 TAAAGCCTGG GGTGCCTAAT GAGTGAGCTA ACTCACATTA ATTGCGTTGC GCTCACTGCC
3001 CGCTTTCCAG TCGGGAAACC TGTCGTGCCA GCTGCATTAA TGAATCGGCC AACGCGCGGG
3061 GAGAGGCGGT TTGCGTATTG GGCGCTCTTC CGCTTCCTCG CTCACTGACT CGCTGCGCTC
3121 GGTCGTTCGG CTGCGGCGAG CGGTATCAGC TCACTCAAAG GCGGTAATAC GGTTATCCAC
3181 AGAATCAGGG GATAACGCAG GAAAGAACAT GTGAGCAAAA GGCCAGCAAA AGGCCAGGAA
3241 CCGTAAAAAG GCCGCGTTGC TGGCGTTTTT CCATAGGCTC CGCCCCCCTG ACGAGCATCA
3301 CAAAAATCGA CGCTCAAGTC AGAGGTGGCG AAACCCGACA GGACTATAAA GATACCAGGC
3361 GTTTCCCCCT GGAAGCTCCC TCGTGCGCTC TCCTGTTCCG ACCCTGCCGC TTACCGGATA
3421 CCTGTCCGCC TTTCTCCCTT CGGGAAGCGT GGCGCTTTCT CATAGCTCAC GCTGTAGGTA
3481 TCTCAGTTCG GTGTAGGTCG TTCGCTCCAA GCTGGGCTGT GTGCACGAAC CCCCCGTTCA
3541 GCCCGACCGC TGCGCCTTAT CCGGTAACTA TCGTCTTGAG TCCAACCCGG TAAGACACGA
3601 CTTATCGCCA CTGGCAGCAG CCACTGGTAA CAGGATTAGC AGAGCGAGGT ATGTAGGCGG
3661 TGCTACAGAG TTCTTGAAGT GGTGGCCTAA CTACGGCTAC ACTAGAAGAA CAGTATTTGG
3721 TATCTGCGCT CTGCTGAAGC CAGTTACCTT CGGAAAAAGA GTTGGTAGCT CTTGATCCGG
3781 CAAACAAACC ACCGCTGGTA GCGGTGGTTT TTTTGTTTGC AAGCAGCAGA TTACGCGCAG
3841 AAAAAAAGGA TCTCAAGAAG ATCCTTTGAT CTTTTCTACG GGGTCTGACG CTCAGTGGAA
3901 CGAAAACTCA CGTTAAGGGA TTTTGGTCAT GAGATTATCA AAAAGGATCT TCACCTAGAT
3961 CCTTTTAAAT TAAAAATGAA GTTTTAAATC AATCTAAAGT ATATATGAGT AAACTTGGTC
4021 TGACAGTTAC CAATGCTTAA TCAGTGAGGC ACCTATCTCA GCGATCTGTC TATTTCGTTC
4081 ATCCATAGTT GCCTGACTCC CCGTCGTGTA GATAACTACG ATACGGGAGG GCTTACCATC
4141 TGGCCCCAGT GCTGCAATGA TACCGCGAGA CCCACGCTCA CCGGCTCCAG ATTTATCAGC
4201 AATAAACCAG CCAGCCGGAA GGGCCGAGCG CAGAAGTGGT CCTGCAACTT TATCCGCCTC
4261 CATCCAGTCT ATTAATTGTT GCCGGGAAGC TAGAGTAAGT AGTTCGCCAG TTAATAGTTT
4321 GCGCAACGTT GTTGCCATTG CTACAGGCAT CGTGGTGTCA CGCTCGTCGT TTGGTATGGC
4381 TTCATTCAGC TCCGGTTCCC AACGATCAAG GCGAGTTACA TGATCCCCCA TGTTGTGCAA
4441 AAAAGCGGTT AGCTCCTTCG GTCCTCCGAT CGTTGTCAGA AGTAAGTTGG CCGCAGTGTT
4501 ATCACTCATG GTTATGGCAG CACTGCATAA TTCTCTTACT GTCATGCCAT CCGTAAGATG
4561 CTTTTCTGTG ACTGGTGAGT ACTCAACCAA GTCATTCTGA GAATAGTGTA TGCGGCGACC
4621 GAGTTGCTCT TGCCCGGCGT CAATACGGGA TAATACCGCG CCACATAGCA GAACTTTAAA
4681 AGTGCTCATC ATTGGAAAAC GTTCTTCGGG GCGAAAACTC TCAAGGATCT TACCGCTGTT
4741 GAGATCCAGT TCGATGTAAC CCACTCGTGC ACCCAACTGA TCTTCAGCAT CTTTTACTTT
4801 CACCAGCGTT TCTGGGTGAG CAAAAACAGG AAGGCAAAAT GCCGCAAAAA AGGGAATAAG
4861 GGCGACACGG AAATGTTGAA TACTCATACT CTTCCTTTTT CAATATTATT GAAGCATTTA
4921 TCAGGGTTAT TGTCTCATGA GCGGATACAT ATTTGAATGT ATTTAGAAAA ATAAACAAAT
4981 AGGGGTTCCG CGCACATTTC CCCGAAAAGT GCCACCTGAC GTCTAAGAAA CCATTATTAT
5041 CATGACATTA ACCTATAAAA ATAGGCGTAT CACGAGGCCC TTTCGTC
pUC57-hFlt3L-GFPnucleic acid sequence (also referred to as pUC57-P501-GFP)
(SEQ ID NO: 4)
      1 TCGCGCGTTT CGGTGATGAC GGTGAAAACC TCTGACACAT GCAGCTCCCG
    51 GAGACGGTCA CAGCTTGTCT GTAAGCGGAT GCCGGGAGCA GACAAGCCCG
  101 TCAGGGCGCG TCAGCGGGTG TTGGCGGGTG TCGGGGCTGG CTTAACTATG
  151 CGGCATCAGA GCAGATTGTA CTGAGAGTGC ACCATATGCG GTGTGAAATA
  201 CCGCACAGAT GCGTAAGGAG AAAATACCGC ATCAGGCGCC ATTCGCCATT
  251 CAGGCTGCGC AACTGTTGGG AAGGGCGATC GGTGCGGGCC TCTTCGCTAT
  301 TACGCCAGCT GGCGAAAGGG GGATGTGCTG CAAGGCGATT AAGTTGGGTA
  351 ACGCCAGGGT TTTCCCAGTC ACGACGTTGT AAAACGACGG CCAGTGAATT
  401 CATATTACGA TGGTAACATA TACGATTTAG CTAAAGATAT AAATGCGATG
  451 TCATTCGACA GTTTTATAAG ATCTCTACAA AATATCTCTT CAAAGAAAGA
  501 TAAACTCACT GTTTATGGAA CCATGGGACT GCTGTCTATT GTCGTAGATA
  551 TTAACAAAGG TTGTGATATA TCCAATATCA AGTTCGCTGC CGGAATAATC
  601 ATTTTAATGG AGTATATTTT TGATGACACG GATATGTCTC ATCTTAAAGT
  651 AGCACTCTAT CGTAGAATAC AGAGACGTGA TGATGTAGAT AGATATTTTT
  701 TTTTCCTAAA CTGATTTCTC TGTTTAAATT CGTAGCGATA TATAAAACAA
  751 CATGTAATTA ATTAATAAAC TTTAAGACAT GTGTGTTATA CTAAGATGGT
  801 TGGCTTATTC CATAGTAGCT TGTGGAATTT ATAAACTTAT GATAGTAAAA
  851 CTAGTACCCA ATATGTAAAG ATGAAAAAGT AAATTACTAT TAACGCCGTC
  901 GGTATTCGTT CATCCATTCA GTAAGCTTAA AAATTGAAAT TTTATTTTTT
  951 TTTTTTGGAA TATAAATAAG CTCGAAGTCG ACGAATTCAT GACAGTGCTG
1001 GCGCCAGCCT GGAGCCCAAC AACCTATCTC CTCCTGCTGC TGCTGCTGAG
1051 CTCGGGACTC AGTGGGACCC AGGACTGCTC CTTCCAACAC AGCCCCATCT
1101 CCTCCGACTT CGCTGTCAAA ATCCGTGAGC TGTCTGACTA CCTGCTTCAA
1151 GATTACCCAG TCACCGTGGC CTCCAACCTG CAGGACGAGG AGCTCTGCGG
1201 GGGCCTCTGG CGGCTGGTCC TGGCACAGCG CTGGATGGAG CGGCTCAAGA
1251 CTGTCGCTGG GTCCAAGATG CAAGGCTTGC TGGAGCGCGT GAACACGGAG
1301 ATACACTTTG TCACCAAATG TGCCTTTCAG CCCCCCCCCA GCTGTCTTCG
1351 CTTCGTCCAG ACCAACATCT CCCGCCTCCT GCAGGAGACC TCCGAGCAGC
1401 TGGTGGCGCT GAAGCCCTGG ATCACTCGCC AGAACTTCTC CCGGTGCCTG
1451 GAGCTGCAGT GTCAGCCCGA CTCCTCAACC CTGCCACCCC CATGGAGTCC
1501 CCGGCCCCTG GAGGCCACAG CCCCGACAGC CCCGCAGCCC CCTCTGCTCC
1551 TCCTACTGCT GCTGCCCGTG GGCCTCCTGC TGCTGGCCGC TGCCTGGTGC
1601 CTGCACTGGC AGAGGACGCG GCGGAGGACA CCCCGCCCTG GGGAGCAGGT
1651 GCCCCCCGTC CCCAGTCCCC AGGACCTGCT GCTTGTGGAG CACTGACTCG
1701 AGTTTACTTG TACAGCTCGT CCATGCCGAG AGTGATCCCG GCGGCGGTCA
1751 CGAACTCCAG CAGGACCATG TGATCGCGCT TCTCGTTGGG GTCTTTGCTC
1801 AGGGCGGACT GGGTGCTCAG GTAGTGGTTG TCGGGCAGCA GCACGGGGCC
1851 GTCGCCGATG GGGGTGTTCT GCTGGTAGTG GTCGGCGAGC TGCACGCTGC
1901 CGTCCTCGAT GTTGTGGCGG ATCTTGAAGT TCACCTTGAT GCCGTTCTTC
1951 TGCTTGTCGG CCATGATATA GACGTTGTGG CTGTTGTAGT TGTACTCCAG
2001 CTTGTGCCCC AGGATGTTGC CGTCCTCCTT GAAGTCGATG CCCTTCAGCT
2051 CGATGCGGTT CACCAGGGTG TCGCCCTCGA ACTTCACCTC GGCGCGGGTC
2101 TTGTAGTTGC CGTCGTCCTT GAAGAAGATG GTGCGCTCCT GGACGTAGCC
2151 TTCGGGCATG GCGGACTTGA AGAAGTCGTG CTGCTTCATG TGGTCGGGGT
2201 AGCGGCTGAA GCACTGCACG CCGTAGGTCA GGGTGGTCAC GAGGGTGGGC
2251 CAGGGCACGG GCAGCTTGCC GGTGGTGCAG ATGAACTTCA GGGTCAGCTT
2301 GCCGTAGGTG GCATCGCCCT CGCCCTCGCC GGACACGCTG AACTTGTGGC
2351 CGTTTACGTC GCCGTCCAGC TCGACCAGGA TGGGCACCAC CCCGGTGAAC
2401 AGCTCCTCGC CCTTGCTCAC CATGGTACCA GGCCTAGATC TGTCGACTTC
2451 GAGCTTATTT ATATTCCAAA AAAAAAAAAT AAAATTTCAA TTTTTCTCGA
2501 GTATGAGTAT AGTGTTAAAT GACACTTACT AAATAGCCAA GGTGATTATT
2551 CGTATTTTTT TAAGGAGTAA CCATGTCCGC AATTAGATTT ATTGCATGTC
2601 TATATCTCAT TTCCATCTTC GGAAATTGTC ATGAGGATCC ATATTATCAA
2651 CCATTTGATA AATTAAACAT TACTCTAGAT ATATACACTT ATGAGGATCT
2701 AGTACCATAC ACCGTAGACA ATGACACAAC TTCTTTCGTT AAGATATACT
2751 TTAAAAATTT TTGGATTACG GTTATGACTA AATGGTGTGC TCCGTTTATT
2801 GATACCGTTA GCGTATACAC ATCTCATGAT AATCTGAATA TACAATTTTA
2851 TAGTAGGGAC GAATATGATA CACAAAGCGA GGATAAAATT TGTACCATTG
2901 ATGTTAAAGC ACGATGCAAA CATCTAACAA AACGAGAAGT TACAGTACAA
2951 CAAGAAGCCT ACAGATAATC TAGATGCATT CGCGAGGTAC CGAATCGGAT
3001 CCCGGGCCCG TCGACTGCAG AGGCCTGCAT GCAAGCTTGG CGTAATCATG
3051 GTCATAGCTG TTTCCTGTGT GAAATTGTTA TCCGCTCACA ATTCCACACA
3101 ACATACGAGC CGGAAGCATA AAGTGTAAAG CCTGGGGTGC CTAATGAGTG
3151 AGCTAACTCA CATTAATTGC GTTGCGCTCA CTGCCCGCTT TCCAGTCGGG
3201 AAACCTGTCG TGCCAGCTGC ATTAATGAAT CGGCCAACGC GCGGGGAGAG
3251 GCGGTTTGCG TATTGGGCGC TCTTCCGCTT CCTCGCTCAC TGACTCGCTG
3301 CGCTCGGTCG TTCGGCTGCG GCGAGCGGTA TCAGCTCACT CAAAGGCGGT
3351 AATACGGTTA TCCACAGAAT CAGGGGATAA CGCAGGAAAG AACATGTGAG
3401 CAAAAGGCCA GCAAAAGGCC AGGAACCGTA AAAAGGCCGC GTTGCTGGCG
3451 TTTTTCCATA GGCTCCGCCC CCCTGACGAG CATCACAAAA ATCGACGCTC
3501 AAGTCAGAGG TGGCGAAACC CGACAGGACT ATAAAGATAC CAGGCGTTTC
3551 CCCCTGGAAG CTCCCTCGTG CGCTCTCCTG TTCCGACCCT GCCGCTTACC
3601 GGATACCTGT CCGCCTTTCT CCCTTCGGGA AGCGTGGCGC TTTCTCATAG
3651 CTCACGCTGT AGGTATCTCA GTTCGGTGTA GGTCGTTCGC TCCAAGCTGG
3701 GCTGTGTGCA CGAACCCCCC GTTCAGCCCG ACCGCTGCGC CTTATCCGGT
3751 AACTATCGTC TTGAGTCCAA CCCGGTAAGA CACGACTTAT CGCCACTGGC
3801 AGCAGCCACT GGTAACAGGA TTAGCAGAGC GAGGTATGTA GGCGGTGCTA
3851 CAGAGTTCTT GAAGTGGTGG CCTAACTACG GCTACACTAG AAGAACAGTA
3901 TTTGGTATCT GCGCTCTGCT GAAGCCAGTT ACCTTCGGAA AAAGAGTTGG
3951 TAGCTCTTGA TCCGGCAAAC AAACCACCGC TGGTAGCGGT GGTTTTTTTG
4001 TTTGCAAGCA GCAGATTACG CGCAGAAAAA AAGGATCTCA AGAAGATCCT
4051 TTGATCTTTT CTACGGGGTC TGACGCTCAG TGGAACGAAA ACTCACGTTA
4101 AGGGATTTTG GTCATGAGAT TATCAAAAAG GATCTTCACC TAGATCCTTT
4151 TAAATTAAAA ATGAAGTTTT AAATCAATCT AAAGTATATA TGAGTAAACT
4201 TGGTCTGACA GTTACCAATG CTTAATCAGT GAGGCACCTA TCTCAGCGAT
4251 CTGTCTATTT CGTTCATCCA TAGTTGCCTG ACTCCCCGTC GTGTAGATAA
4301 CTACGATACG GGAGGGCTTA CCATCTGGCC CCAGTGCTGC AATGATACCG
4351 CGAGACCCAC GCTCACCGGC TCCAGATTTA TCAGCAATAA ACCAGCCAGC
4401 CGGAAGGGCC GAGCGCAGAA GTGGTCCTGC AACTTTATCC GCCTCCATCC
4451 AGTCTATTAA TTGTTGCCGG GAAGCTAGAG TAAGTAGTTC GCCAGTTAAT
4501 AGTTTGCGCA ACGTTGTTGC CATTGCTACA GGCATCGTGG TGTCACGCTC
4551 GTCGTTTGGT ATGGCTTCAT TCAGCTCCGG TTCCCAACGA TCAAGGCGAG
4601 TTACATGATC CCCCATGTTG TGCAAAAAAG CGGTTAGCTC CTTCGGTCCT
4651 CCGATCGTTG TCAGAAGTAA GTTGGCCGCA GTGTTATCAC TCATGGTTAT
4701 GGCAGCACTG CATAATTCTC TTACTGTCAT GCCATCCGTA AGATGCTTTT
4751 CTGTGACTGG TGAGTACTCA ACCAAGTCAT TCTGAGAATA GTGTATGCGG
4801 CGACCGAGTT GCTCTTGCCC GGCGTCAATA CGGGATAATA CCGCGCCACA
4851 TAGCAGAACT TTAAAAGTGC TCATCATTGG AAAACGTTCT TCGGGGCGAA
4901 AACTCTCAAG GATCTTACCG CTGTTGAGAT CCAGTTCGAT GTAACCCACT
4951 CGTGCACCCA ACTGATCTTC AGCATCTTTT ACTTTCACCA GCGTTTCTGG
5001 GTGAGCAAAA ACAGGAAGGC AAAATGCCGC AAAAAAGGGA ATAAGGGCGA
5051 CACGGAAATG TTGAATACTC ATACTCTTCC TTTTTCAATA TTATTGAAGC
5101 ATTTATCAGG GTTATTGTCT CATGAGCGGA TACATATTTG AATGTATTTA
5151 GAAAAATAAA CAAATAGGGG TTCCGCGCAC ATTTCCCCGA AAAGTGCCAC
5201 CTGACGTCTA AGAAACCATT ATTATCATGA CATTAACCTA TAAAAATAGG
5251 CGTATCACGA GGCCCTTTCG TC
```

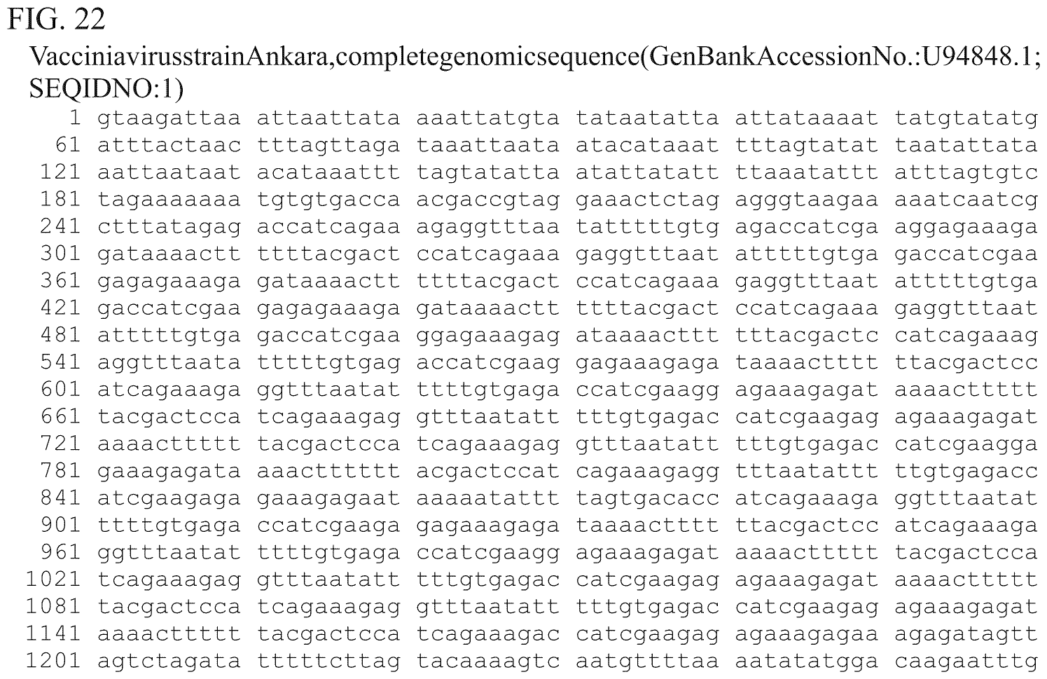

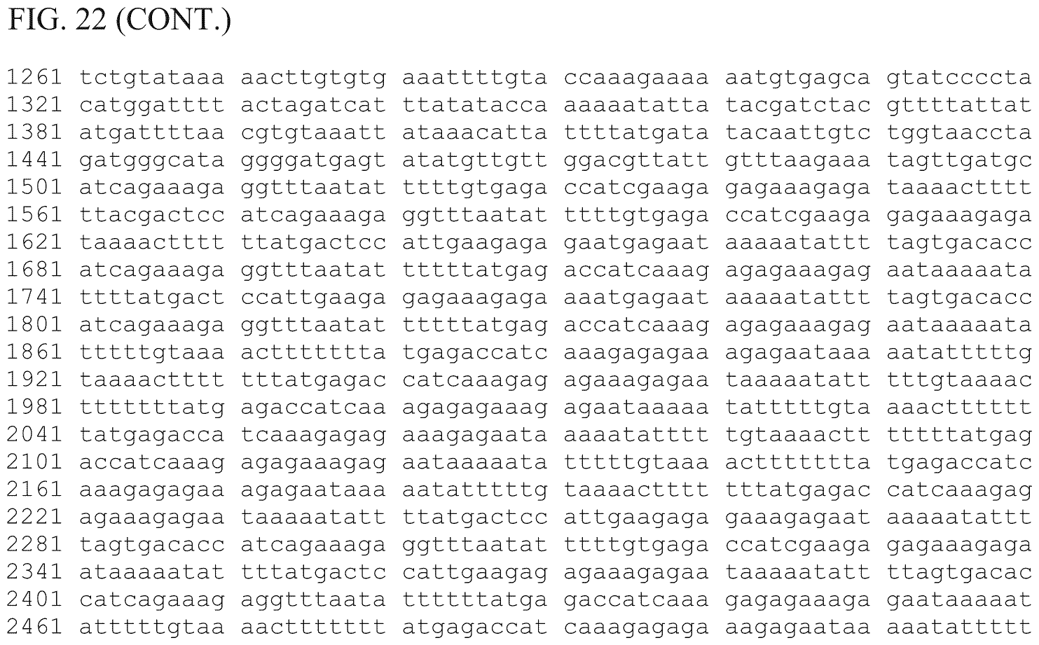

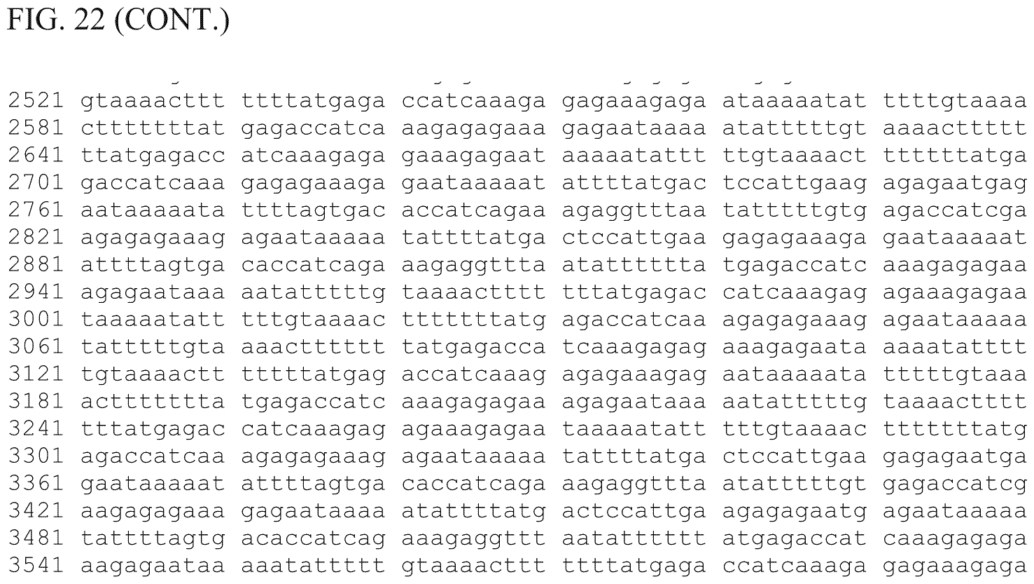

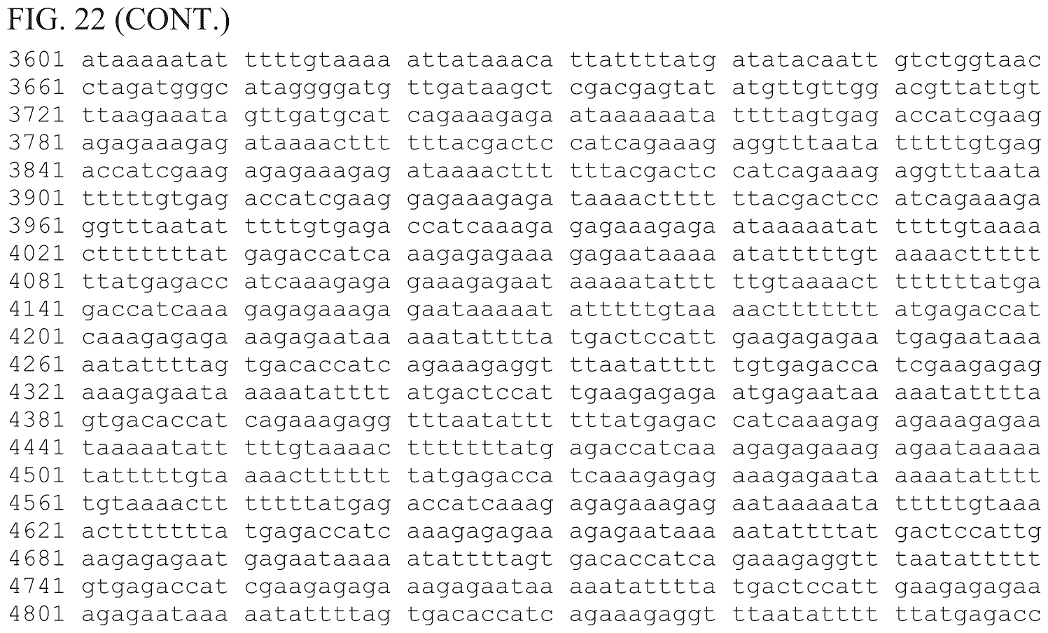

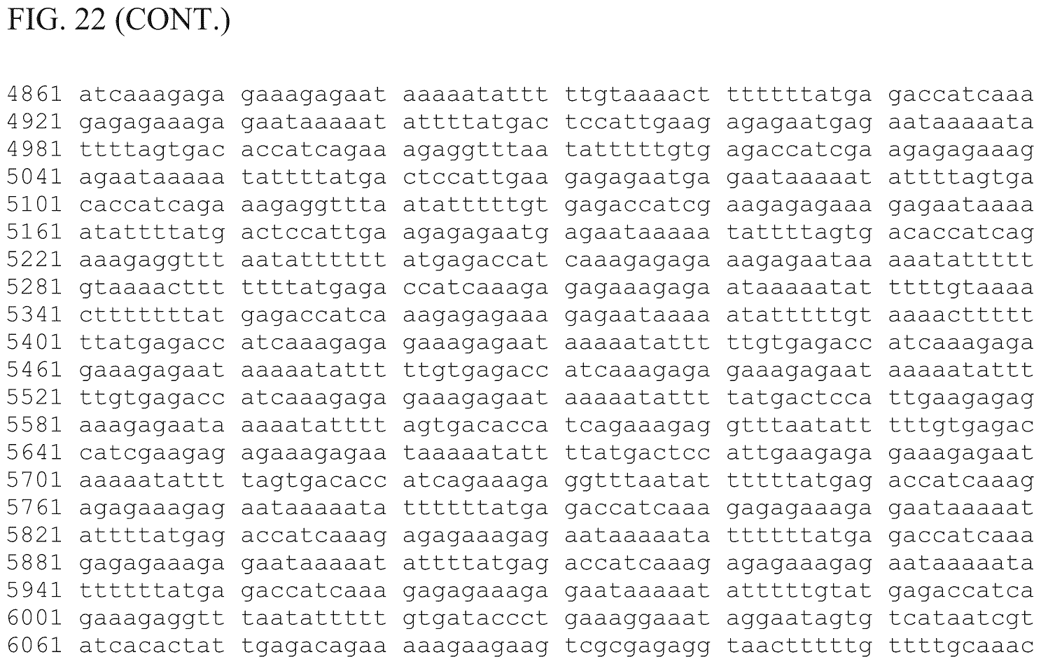

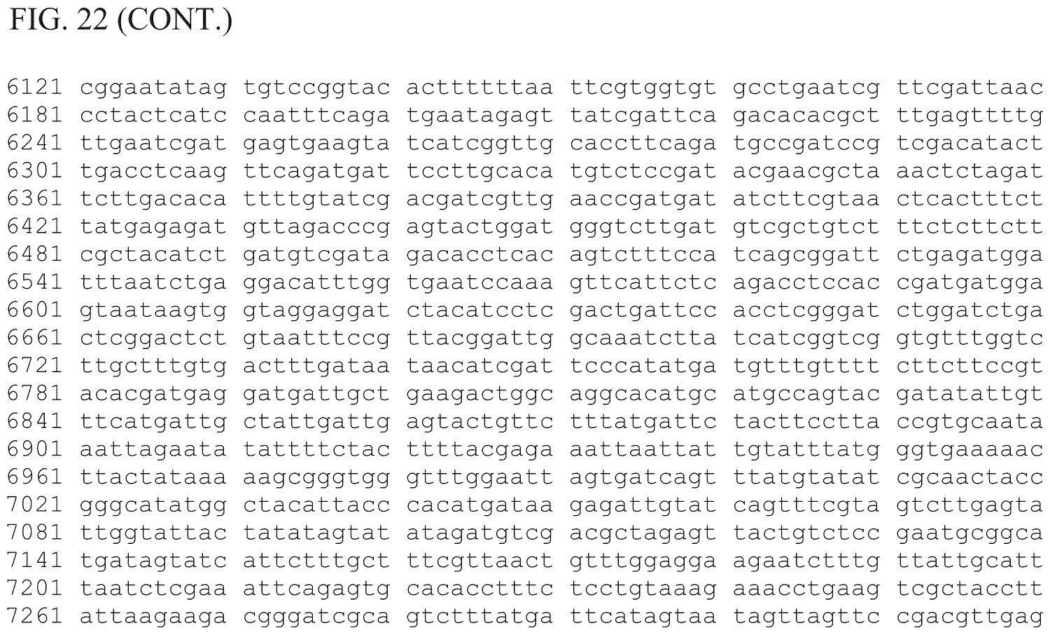

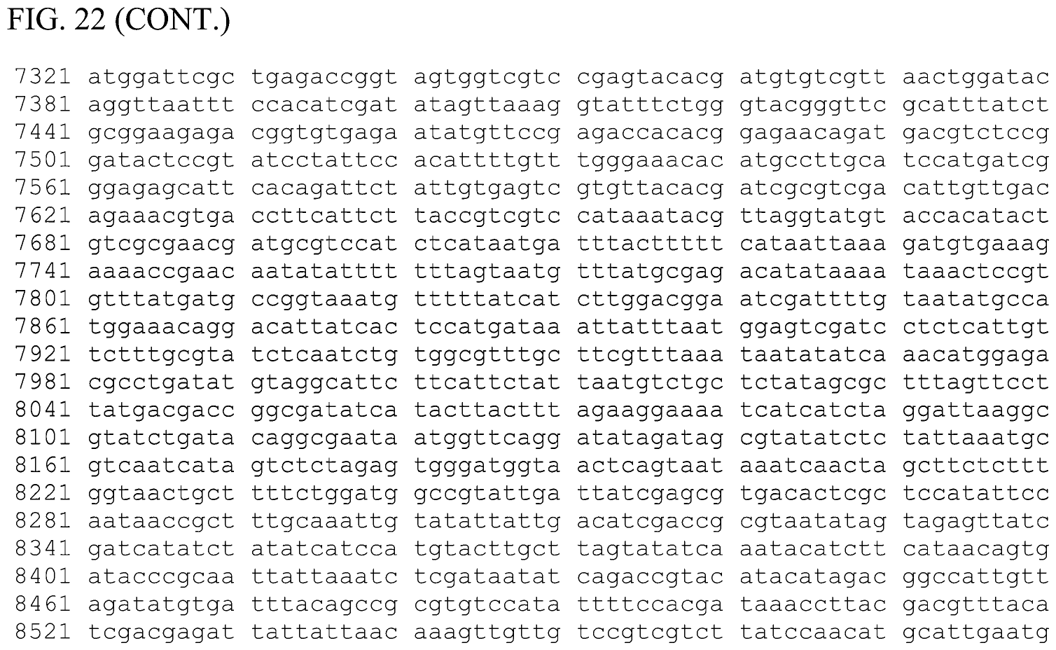

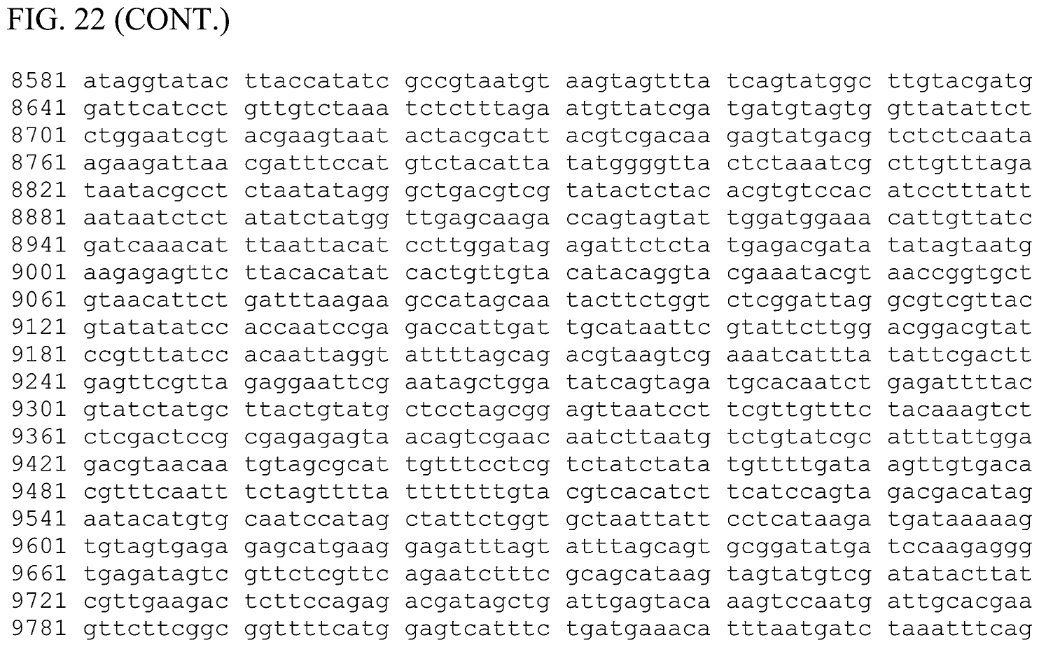

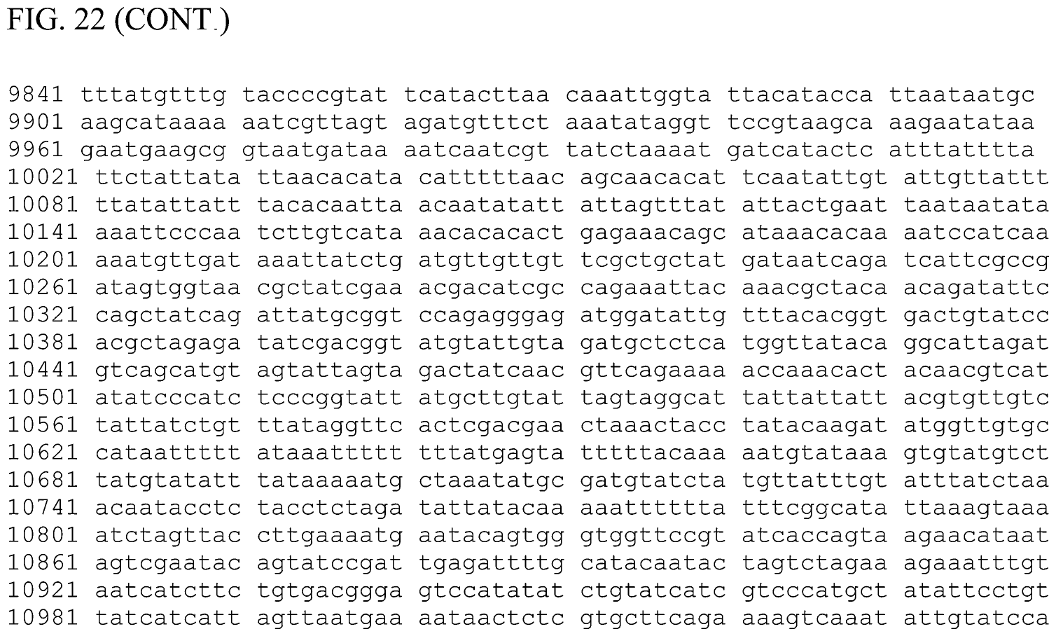

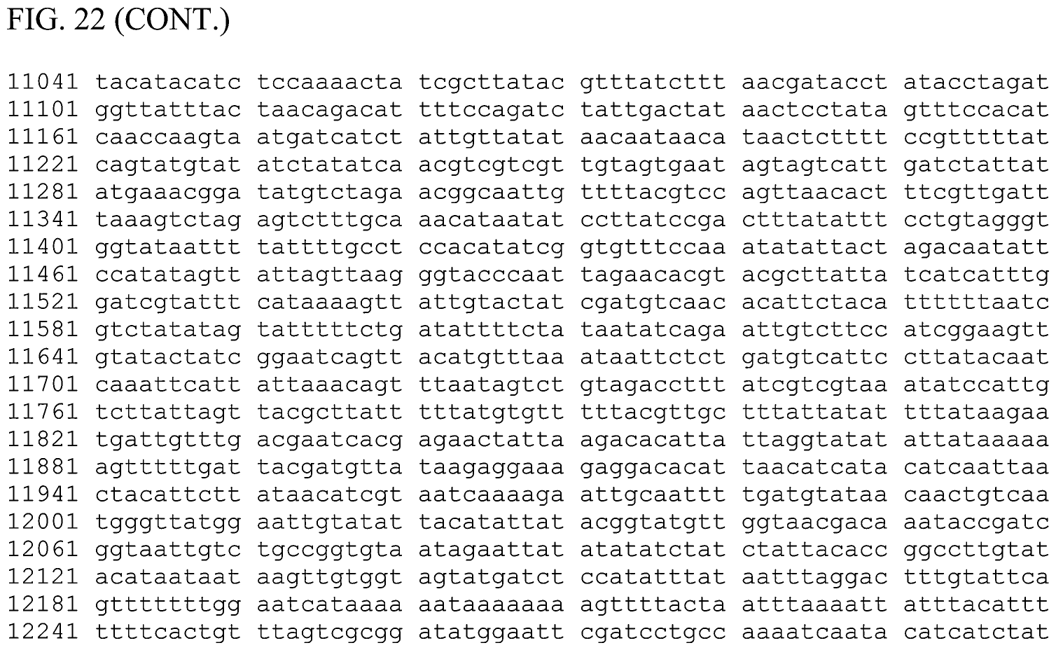

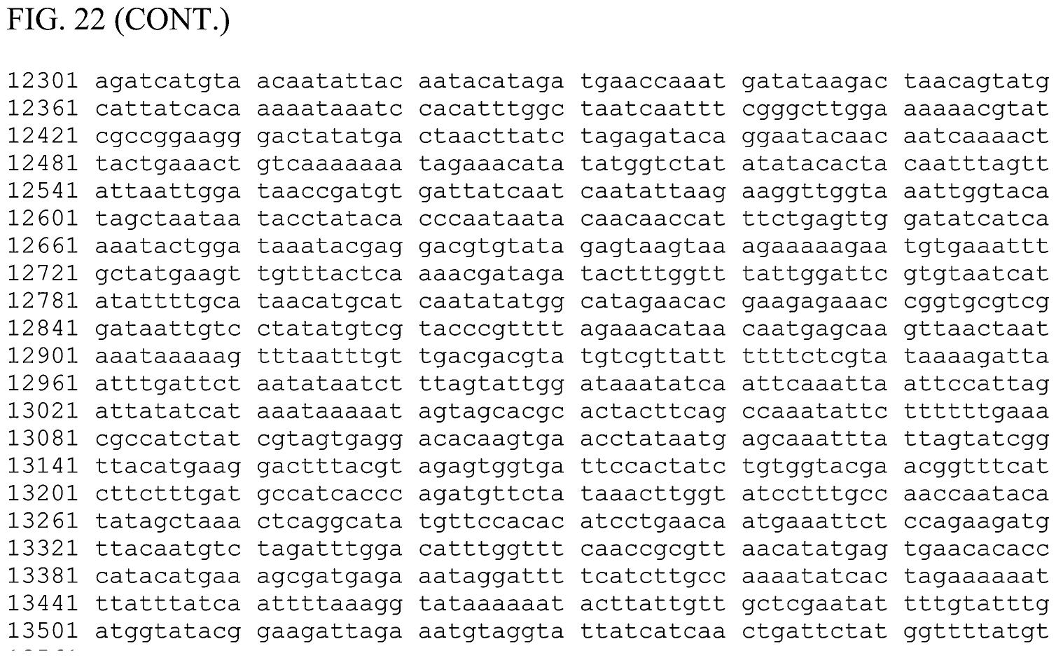

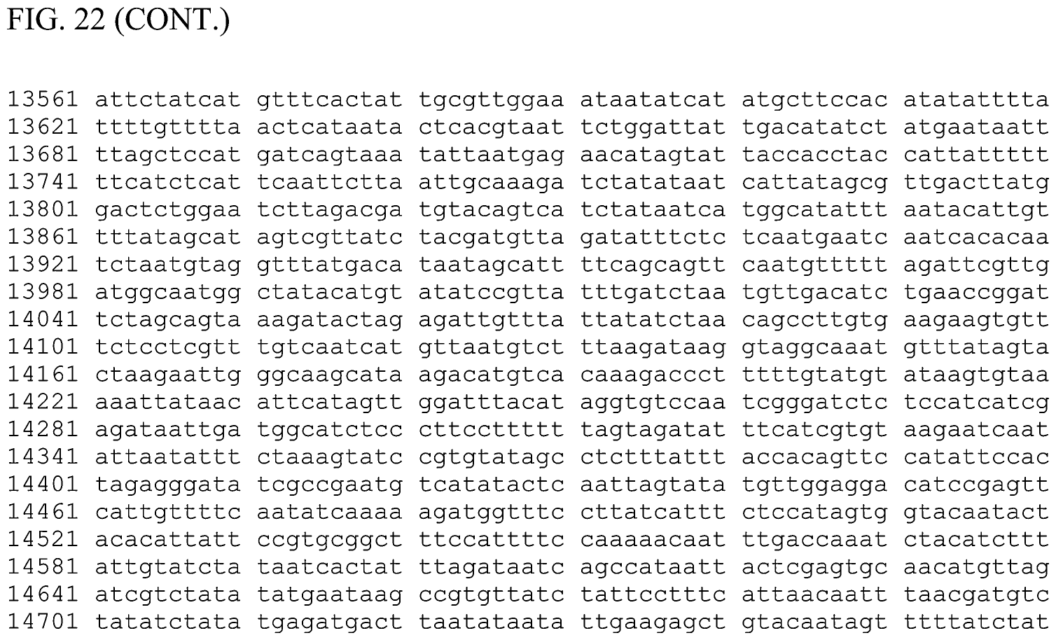

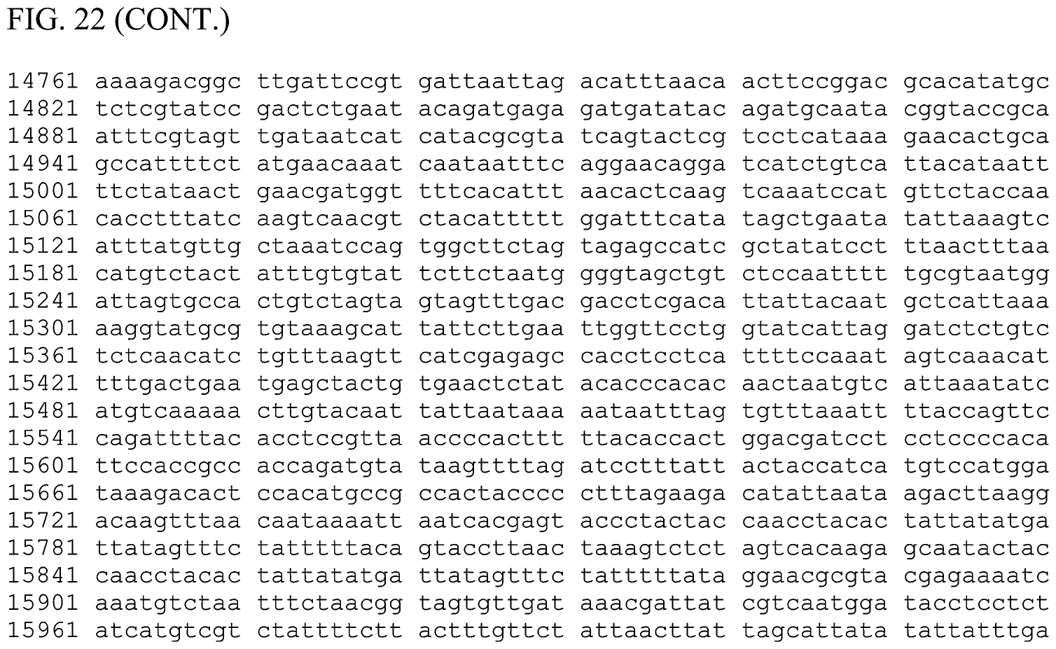

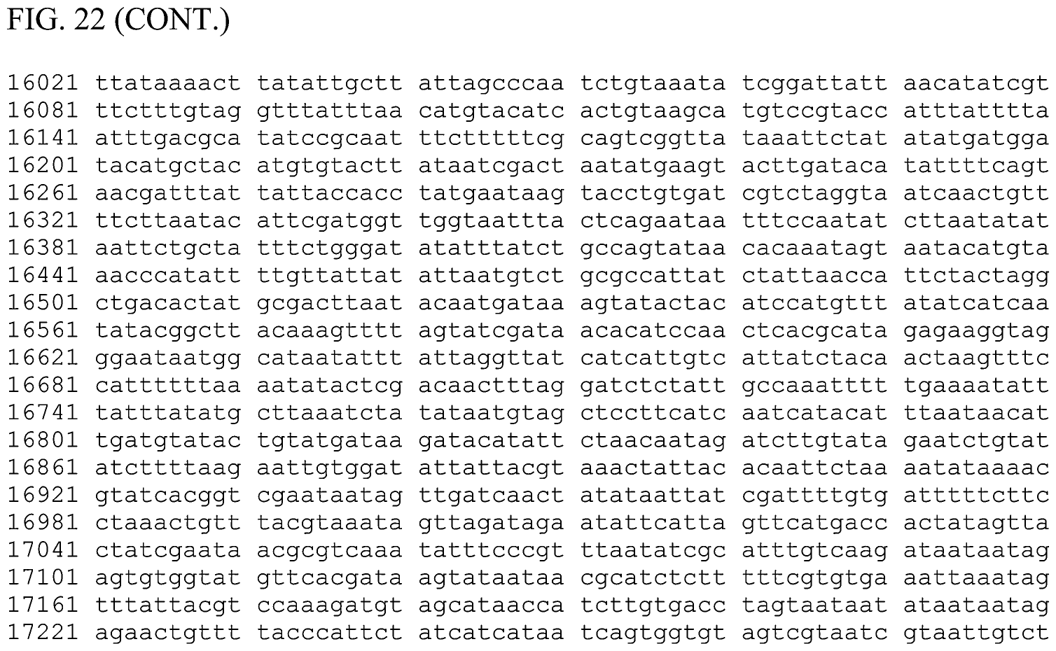

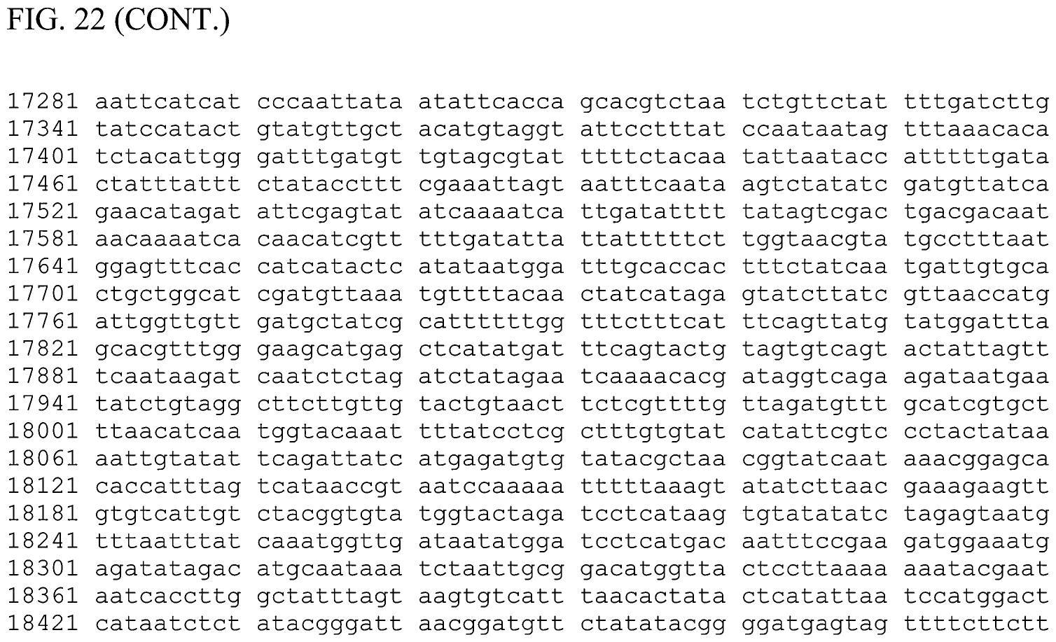

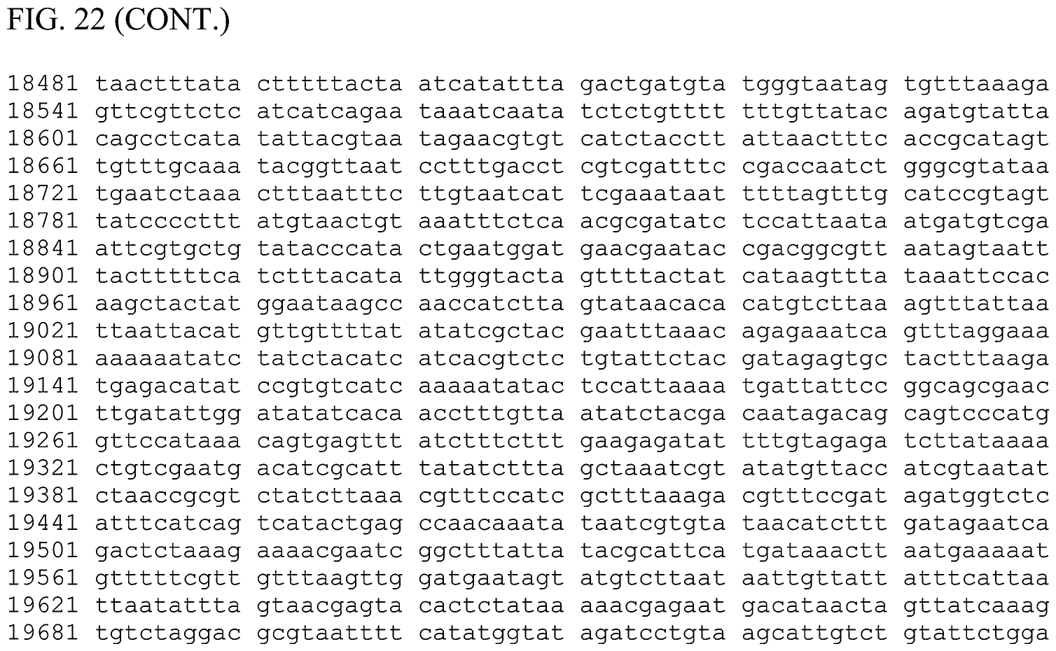

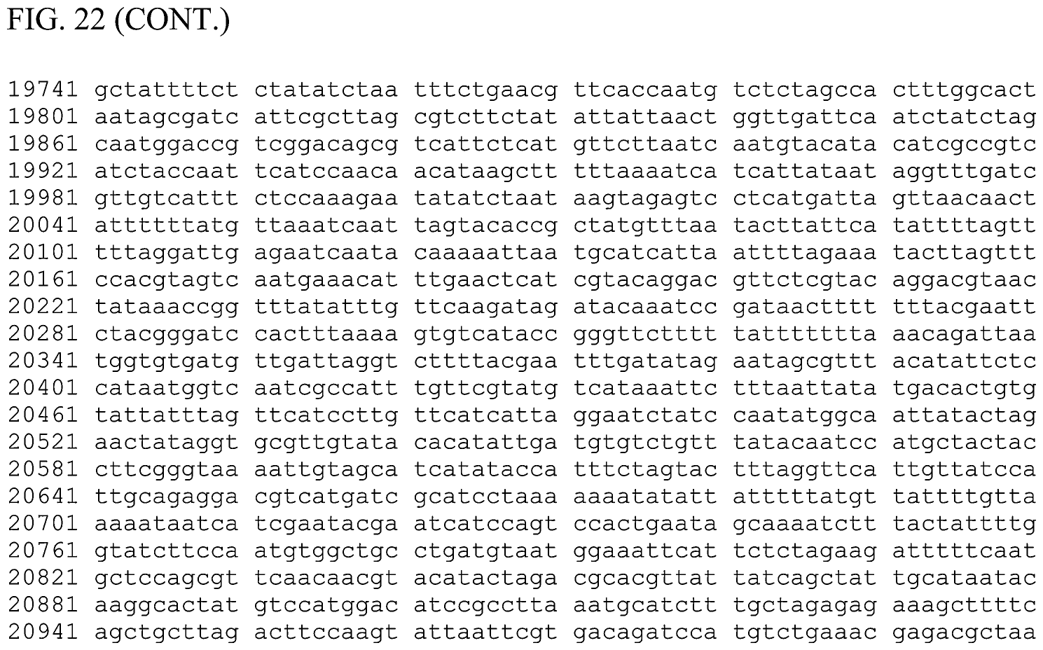

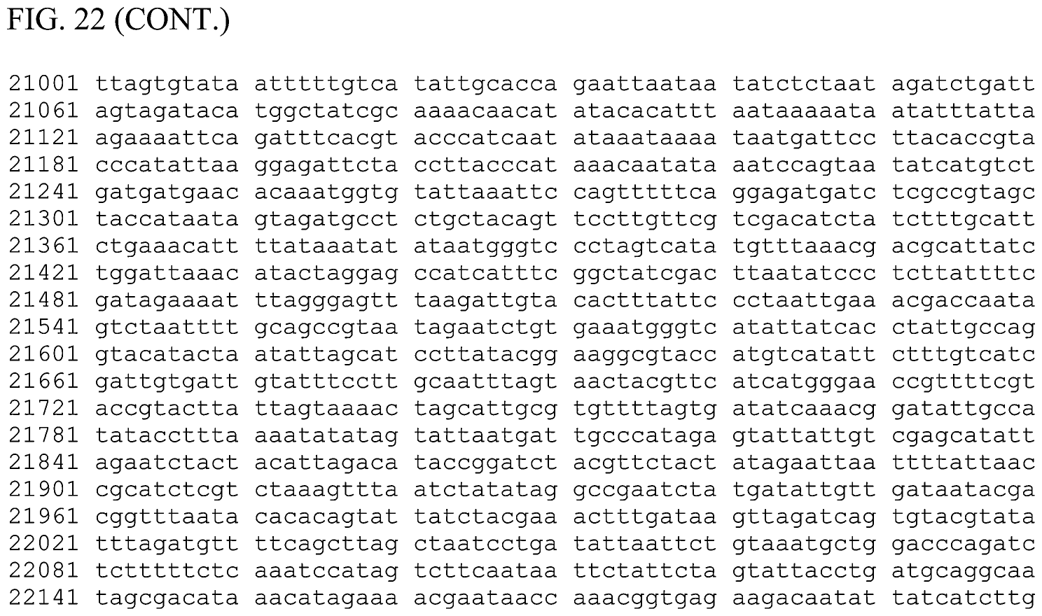

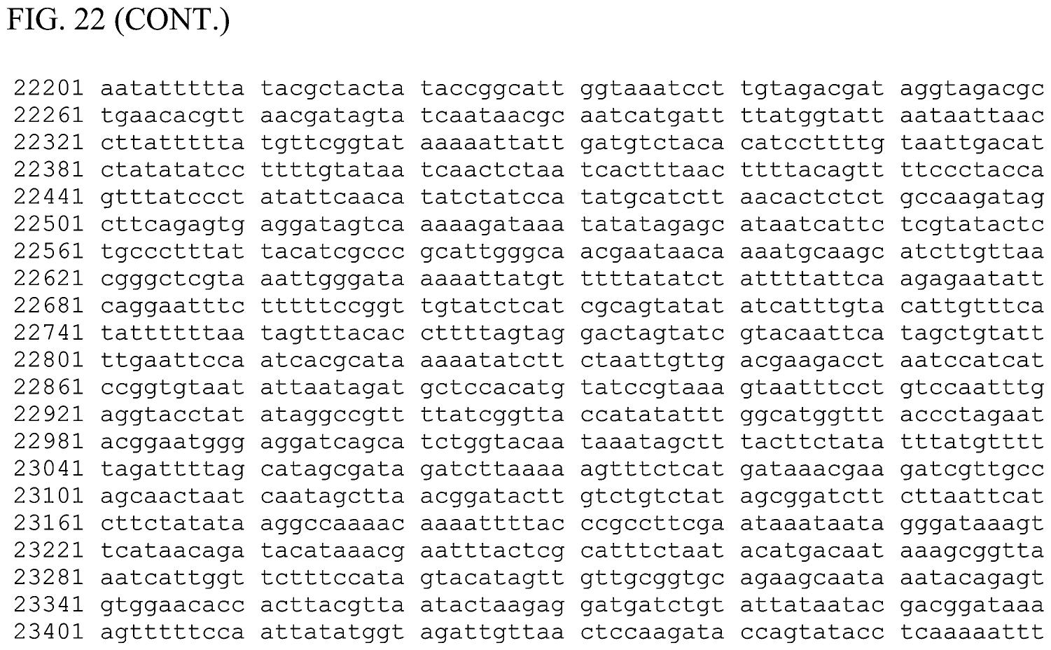

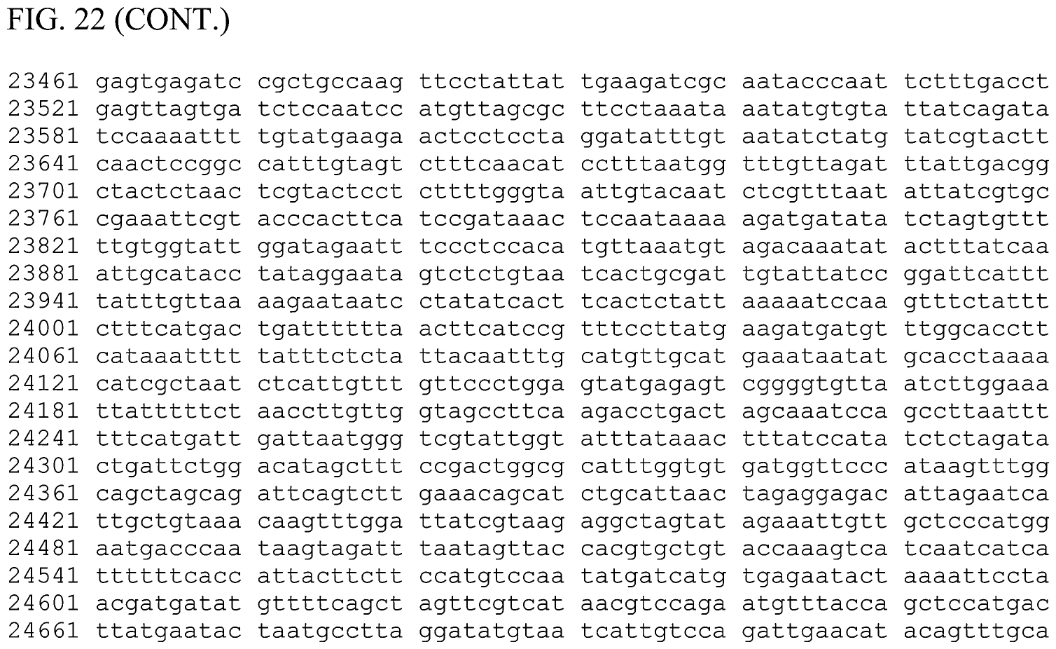

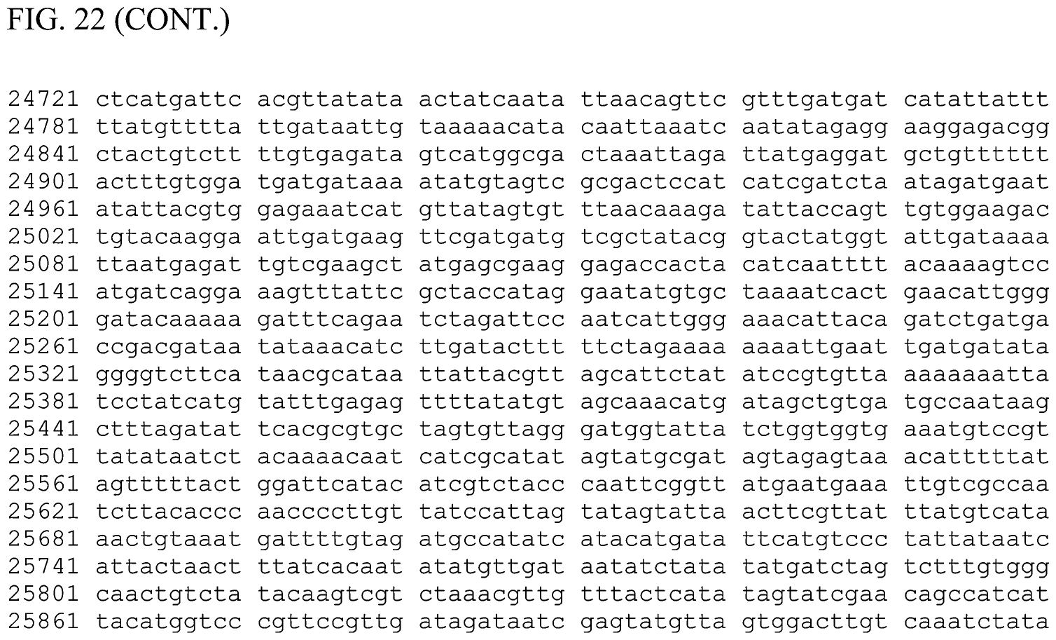

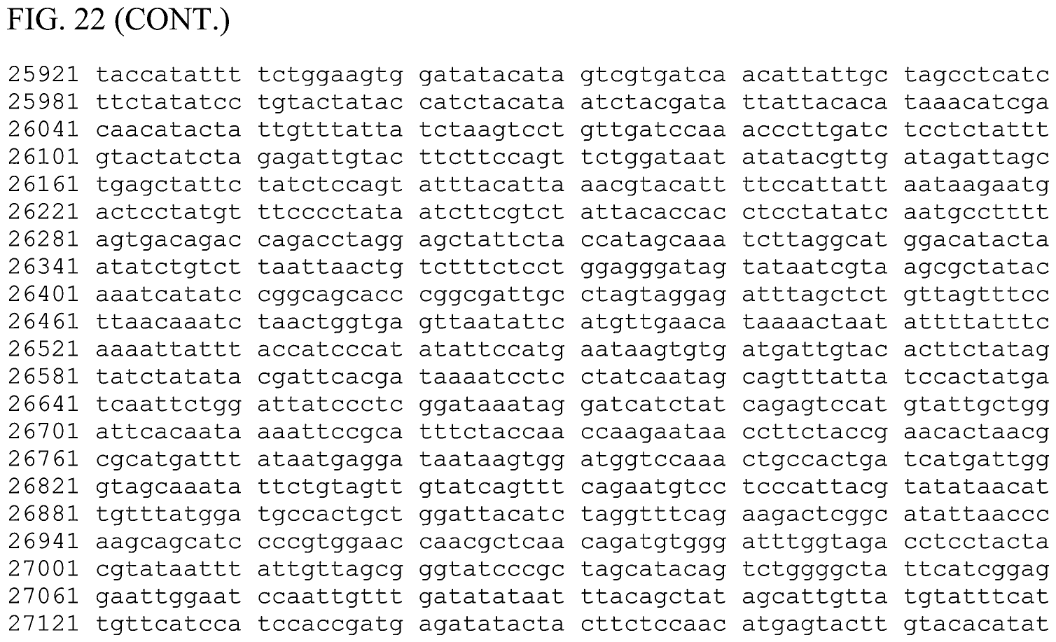

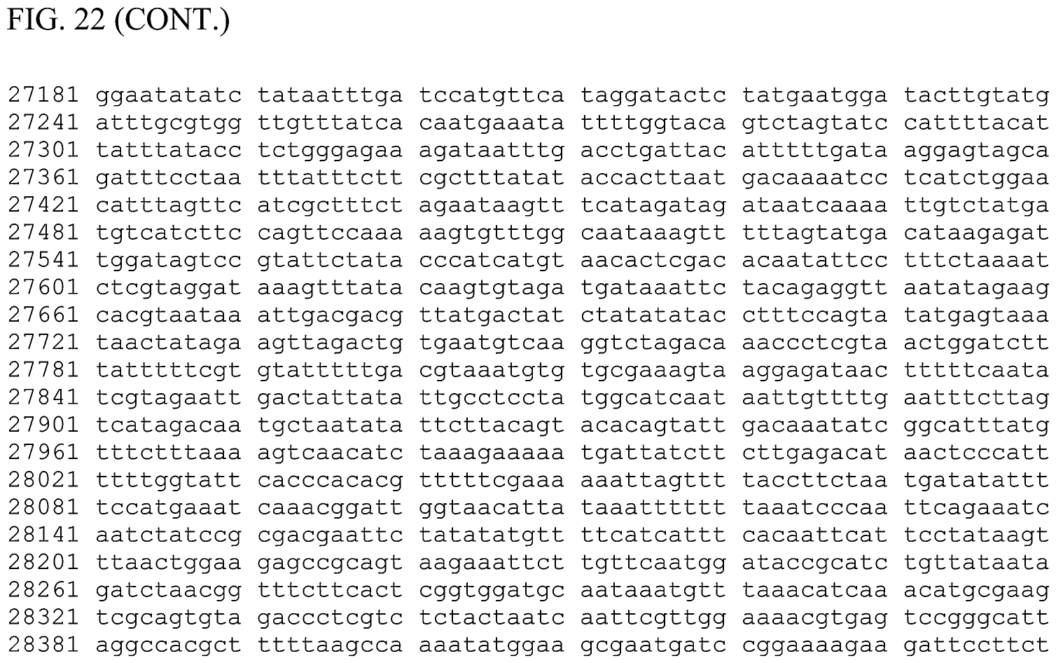

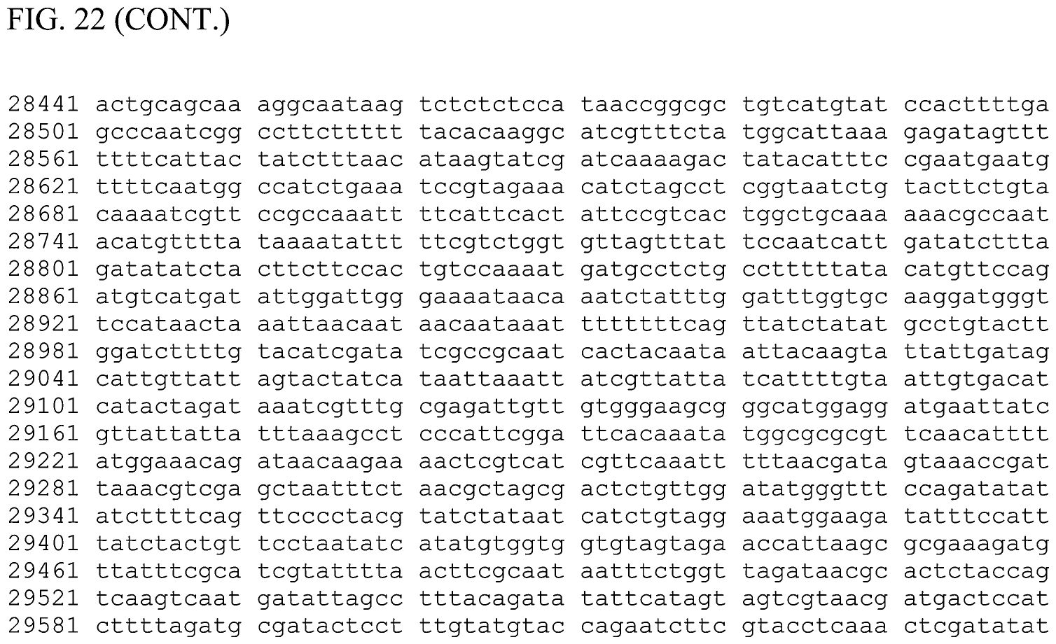

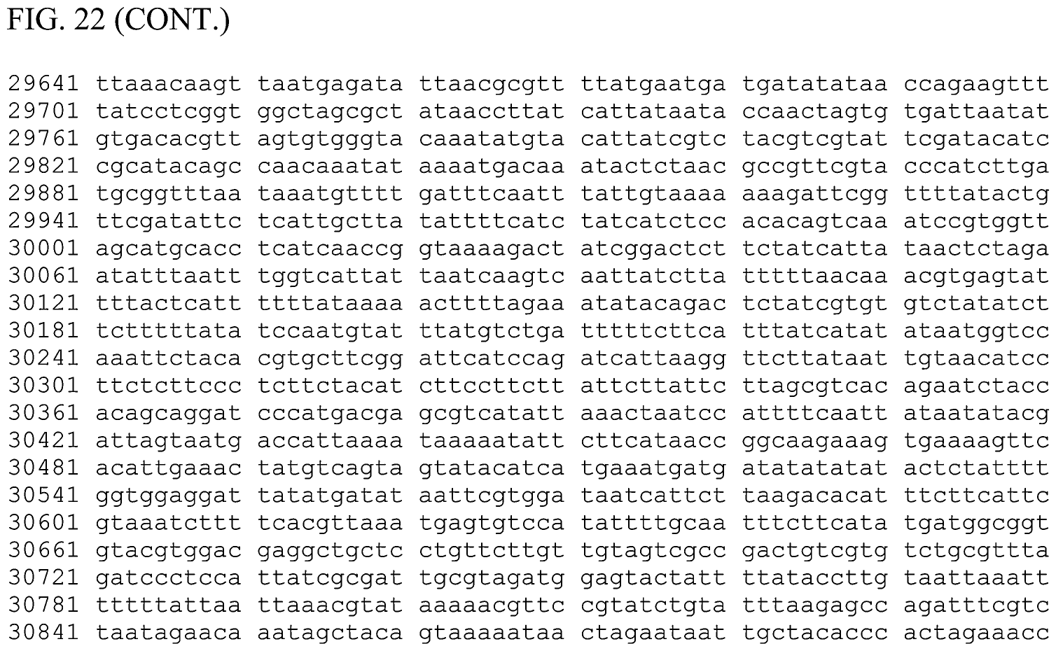

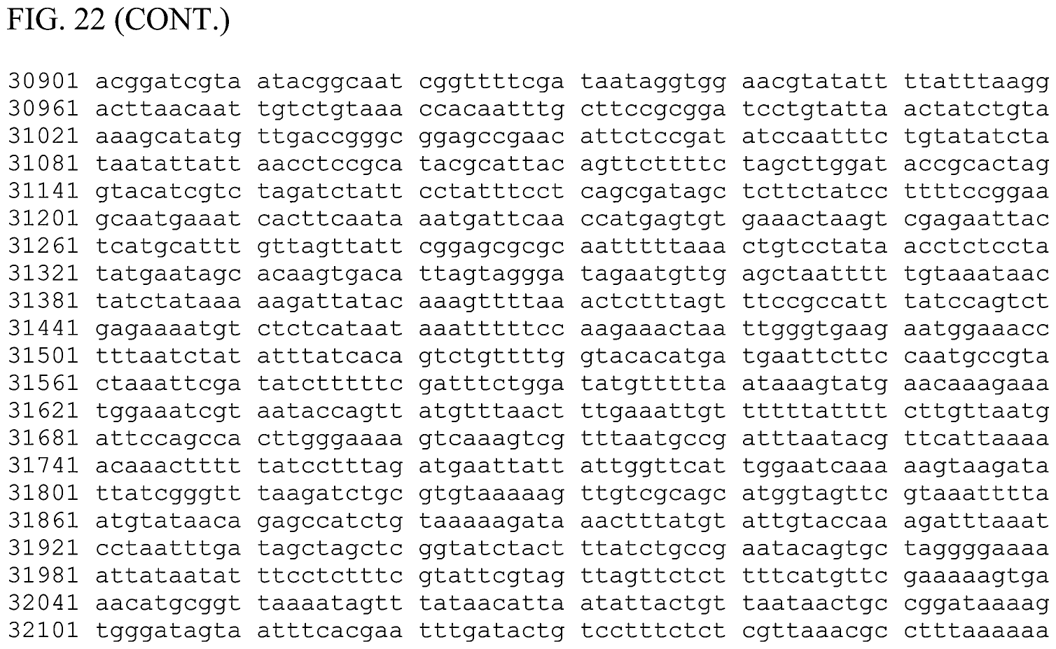

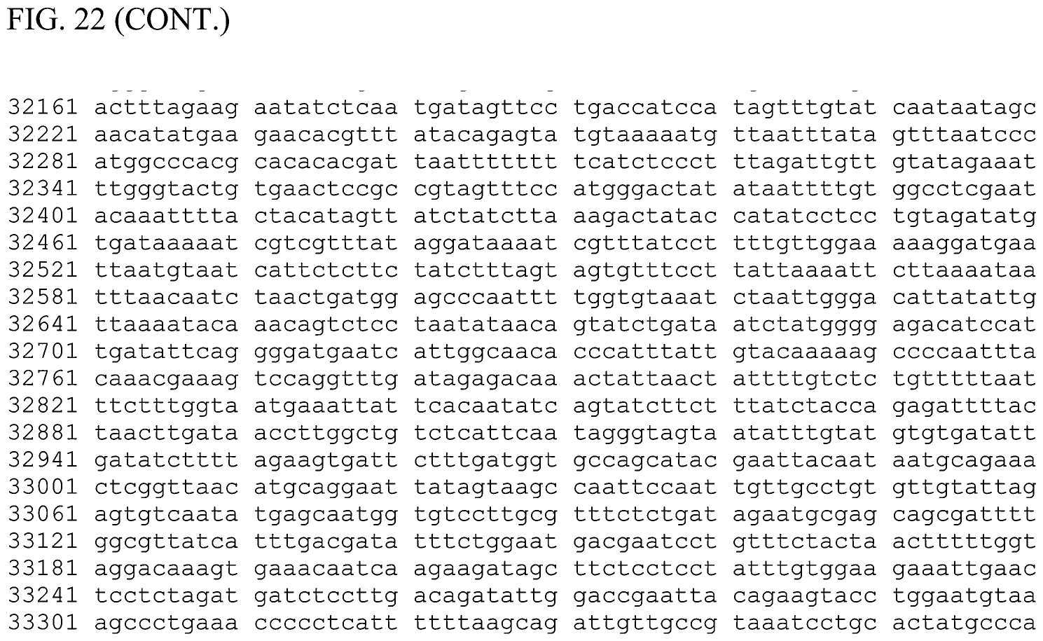

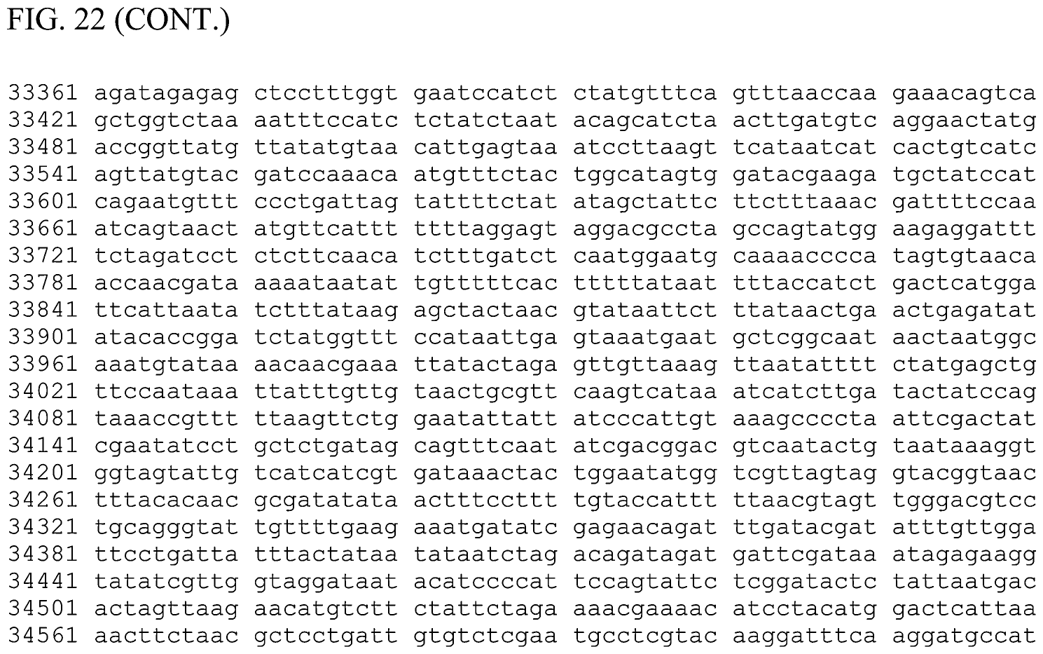

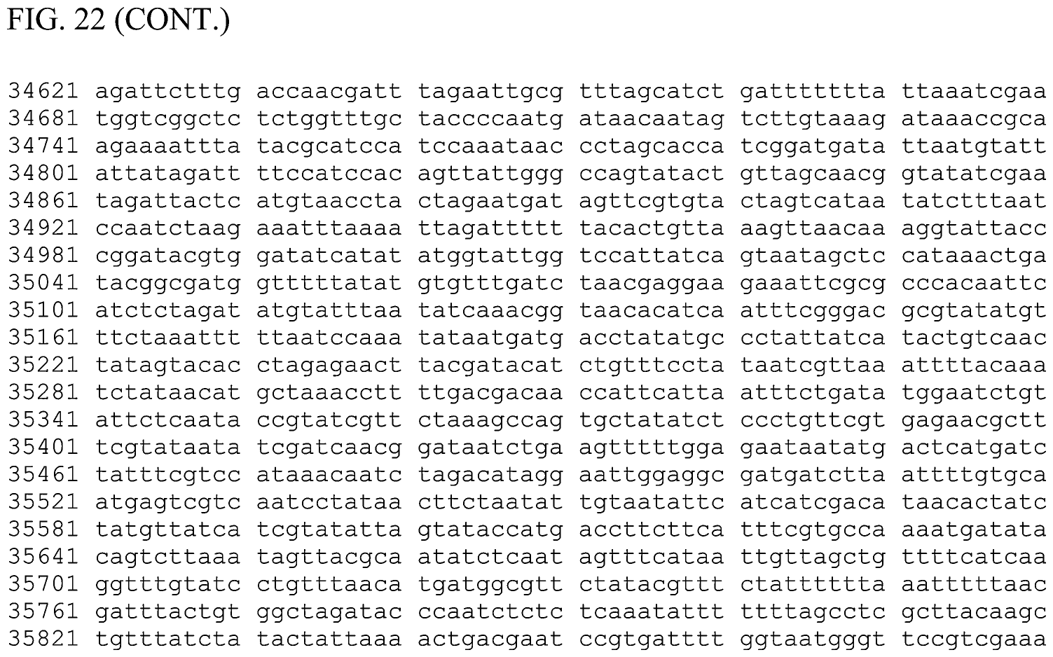

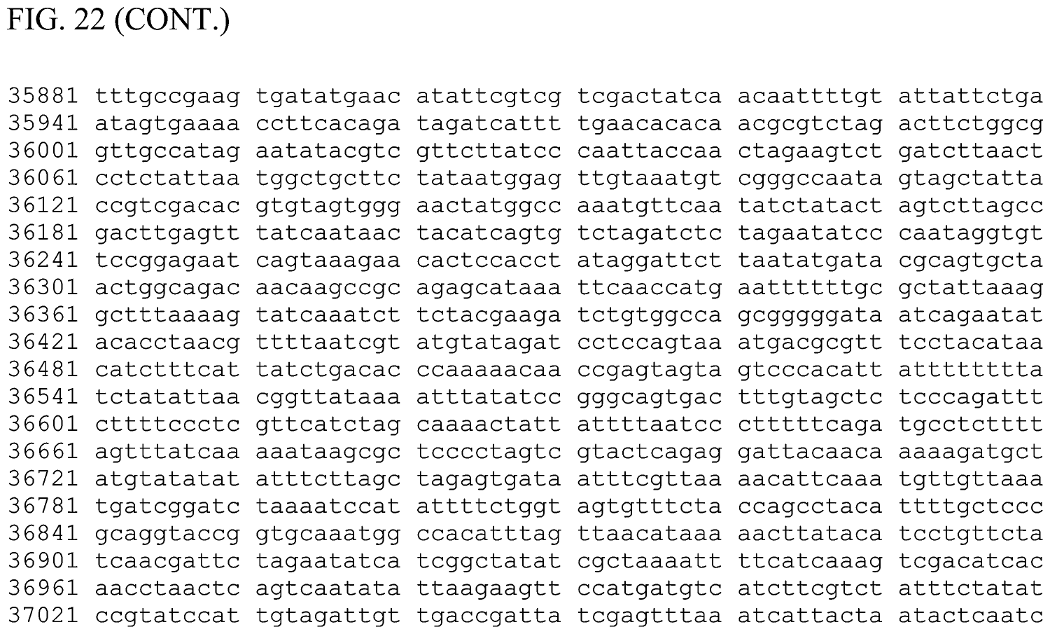

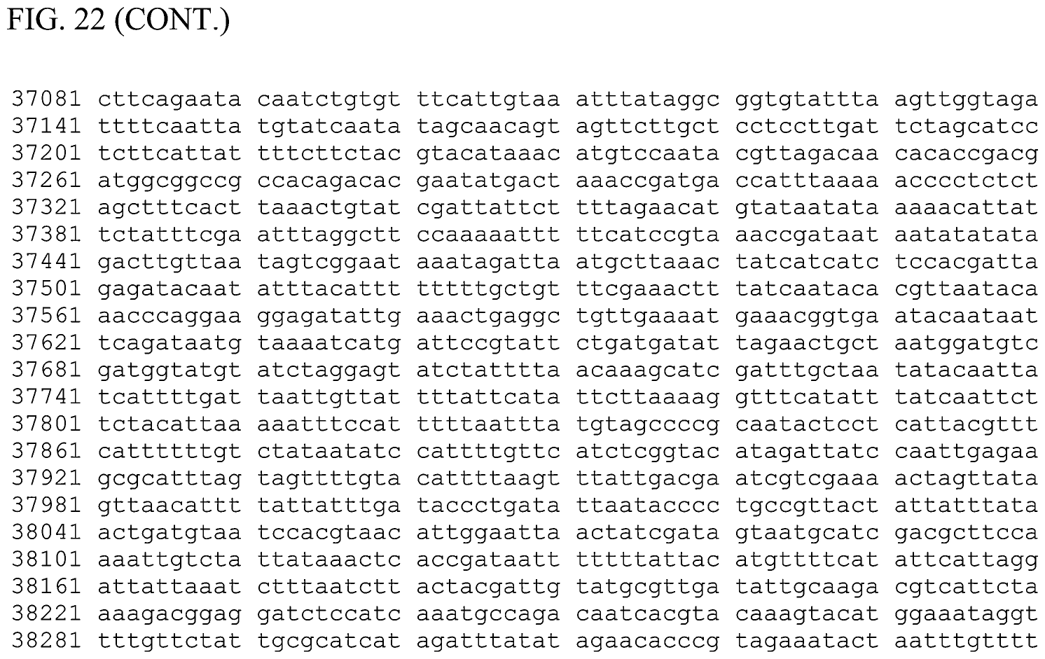

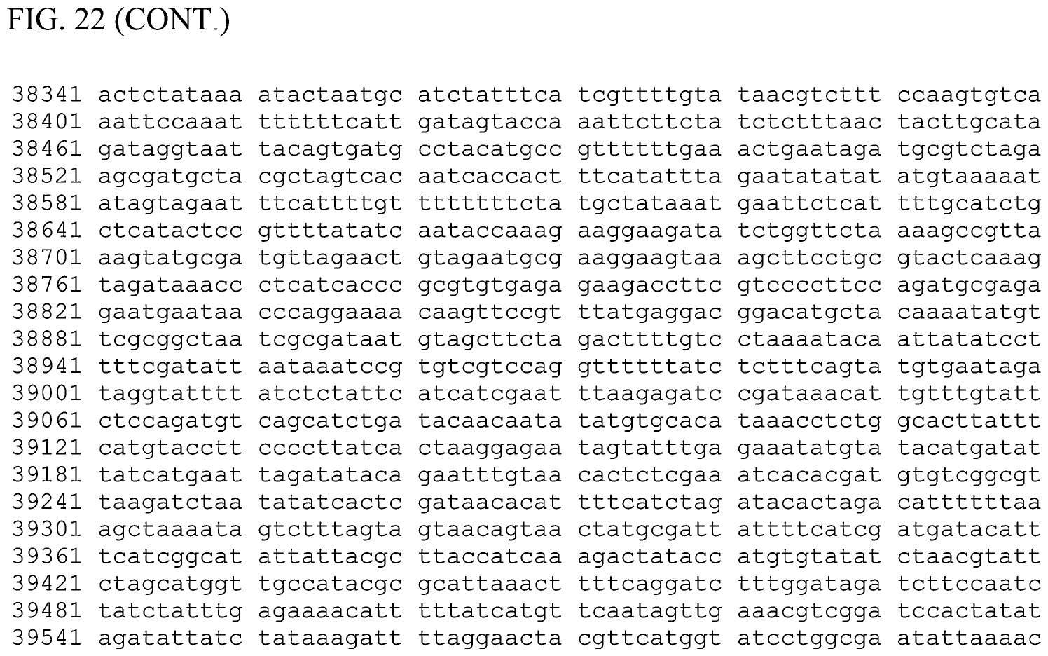

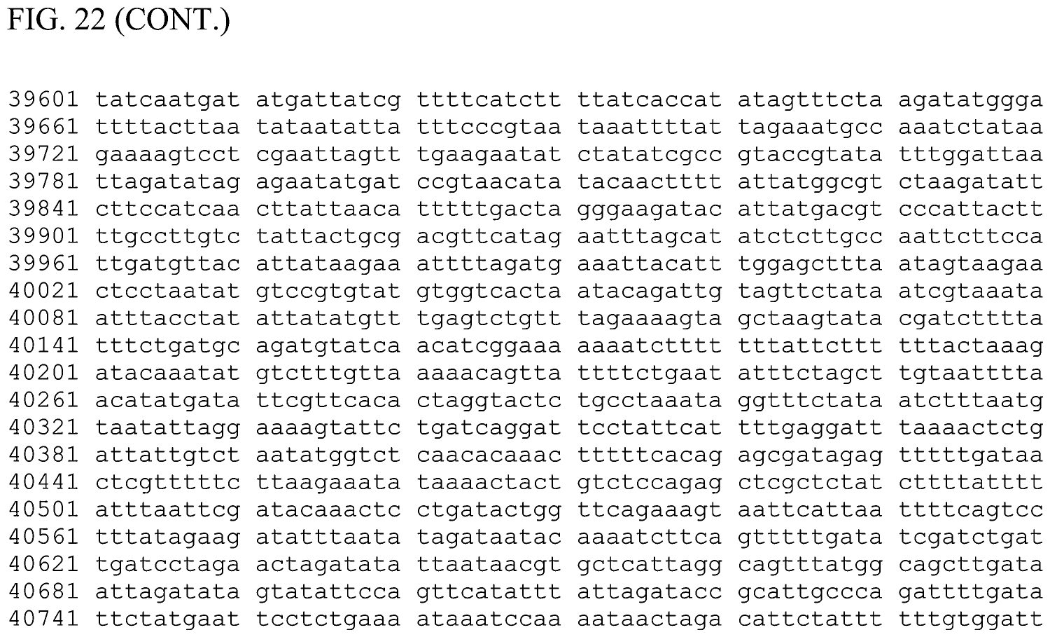

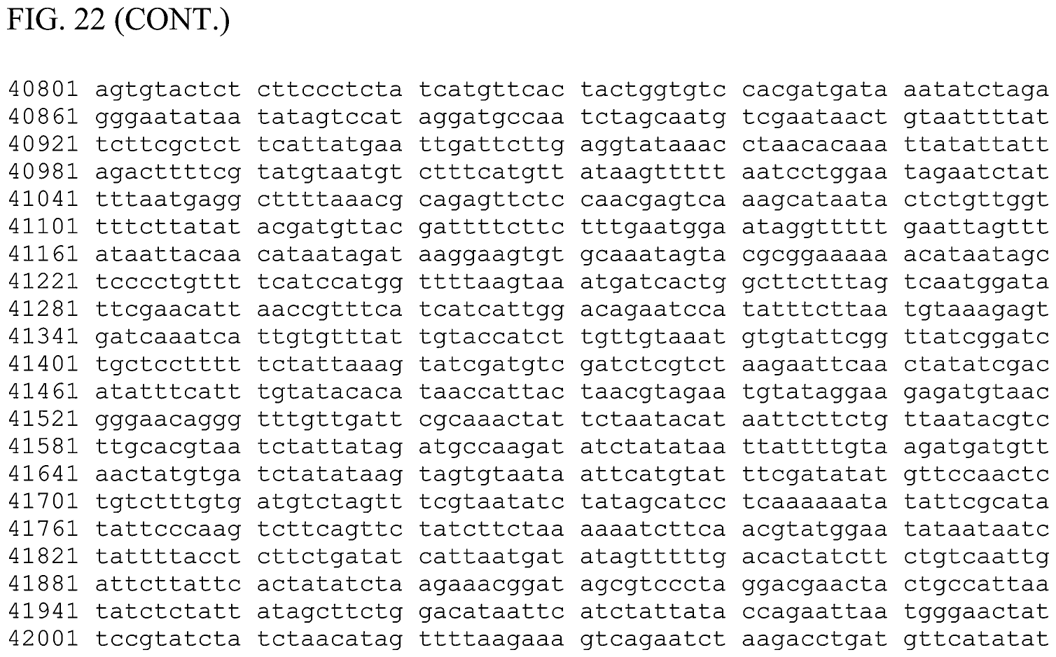

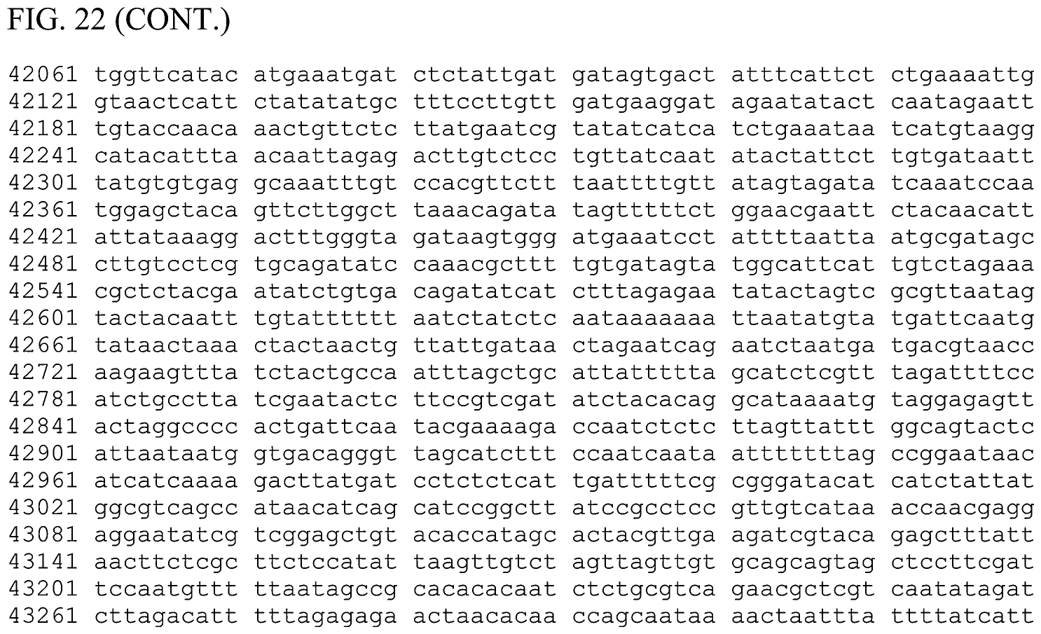

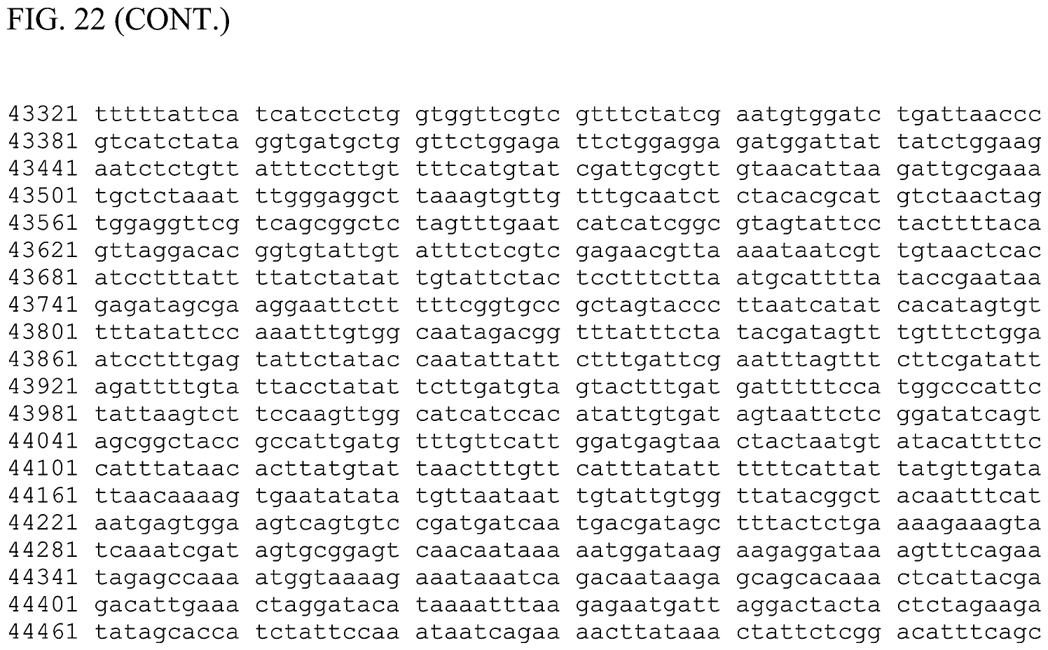

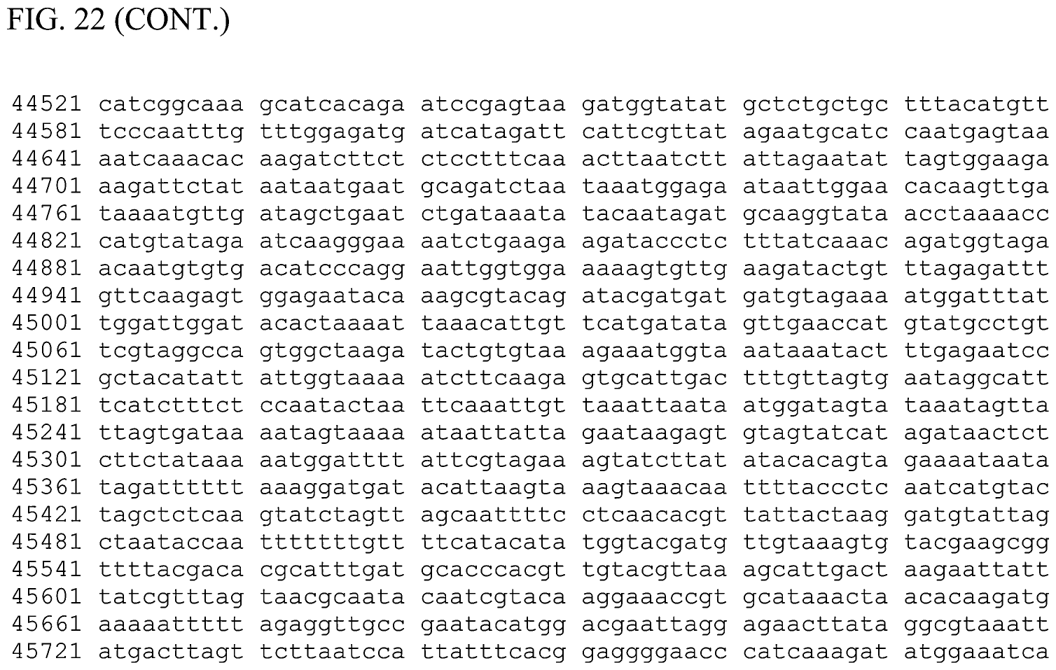

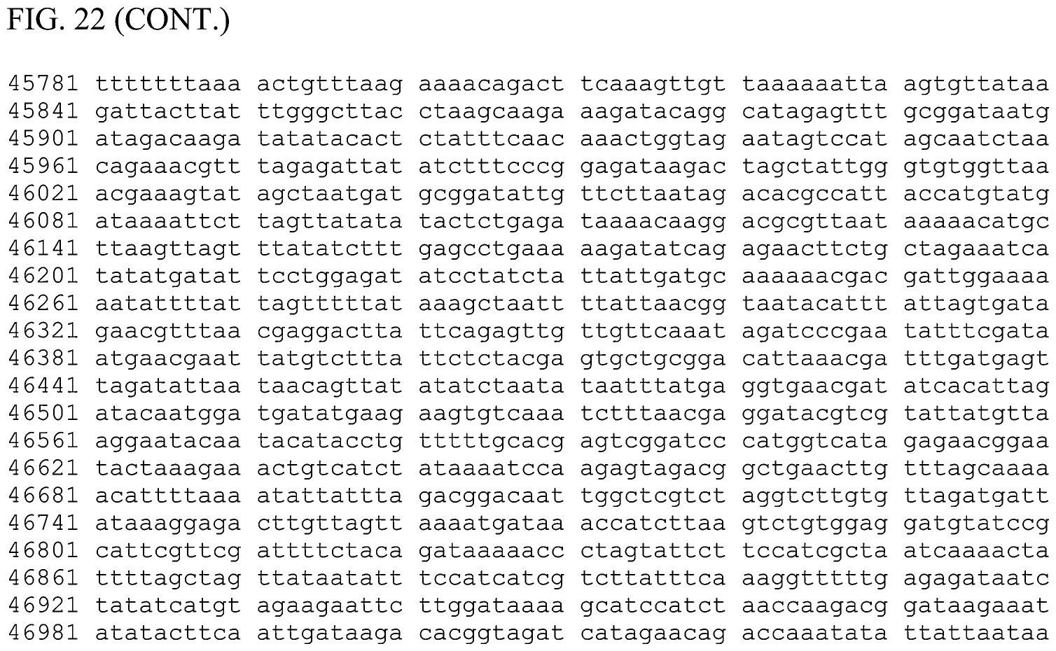

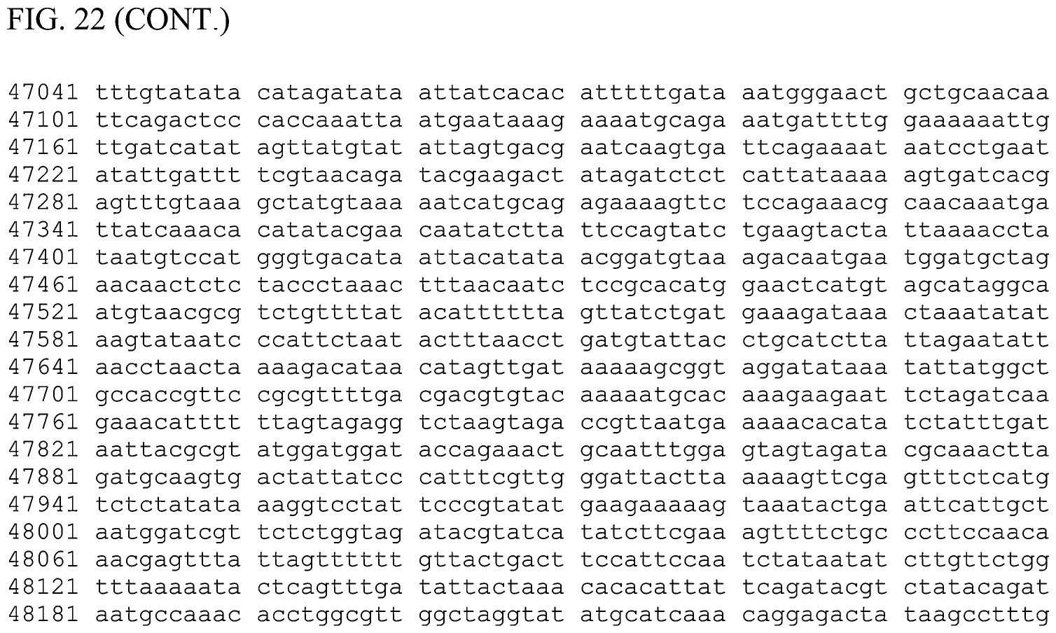

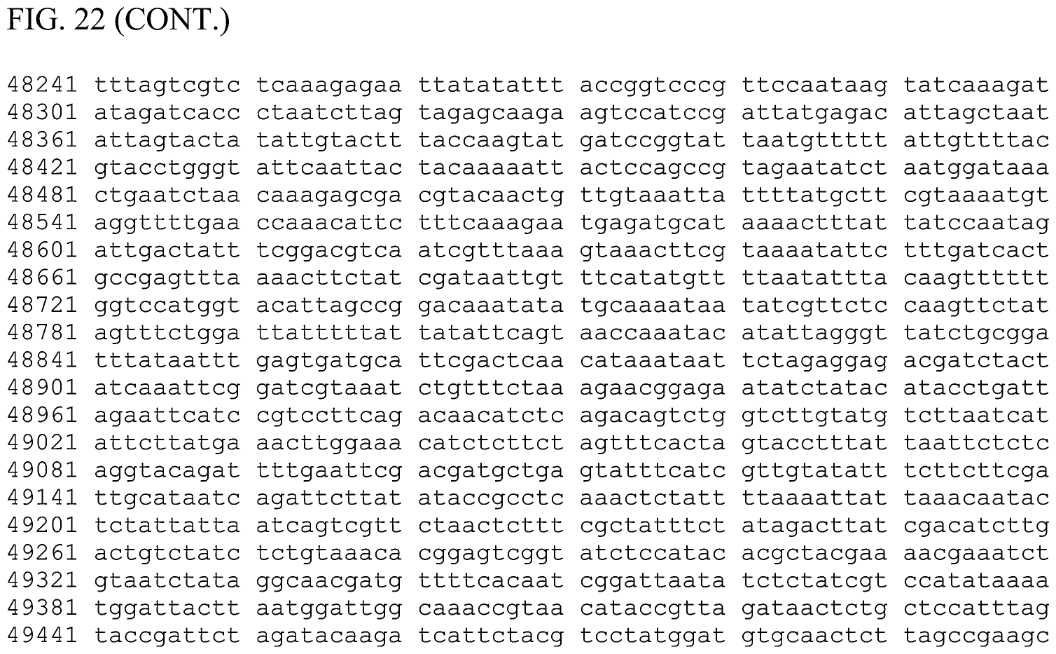

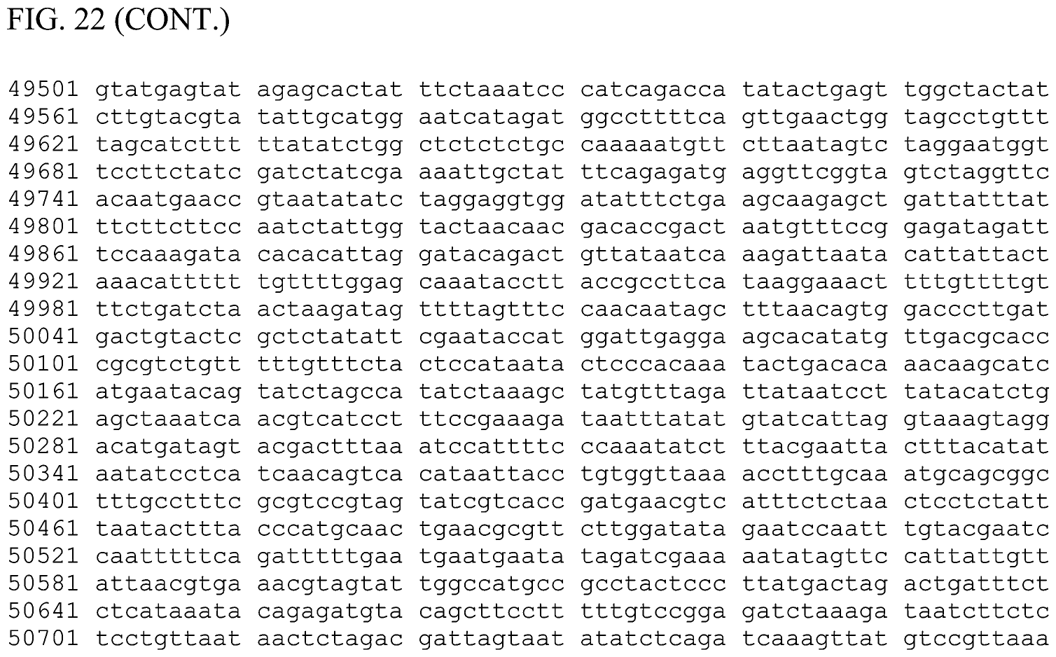

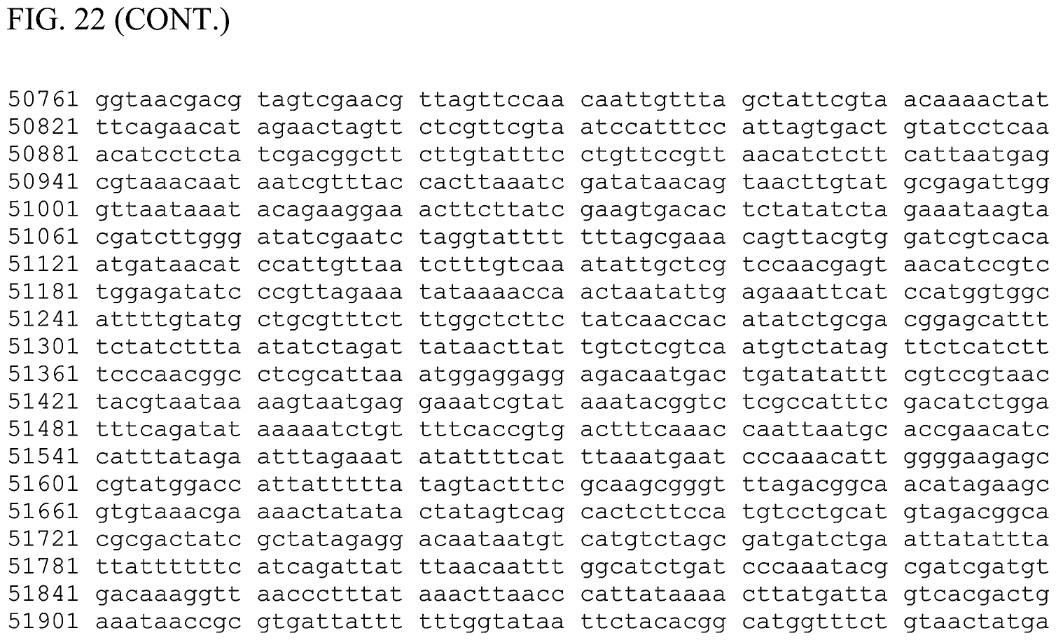

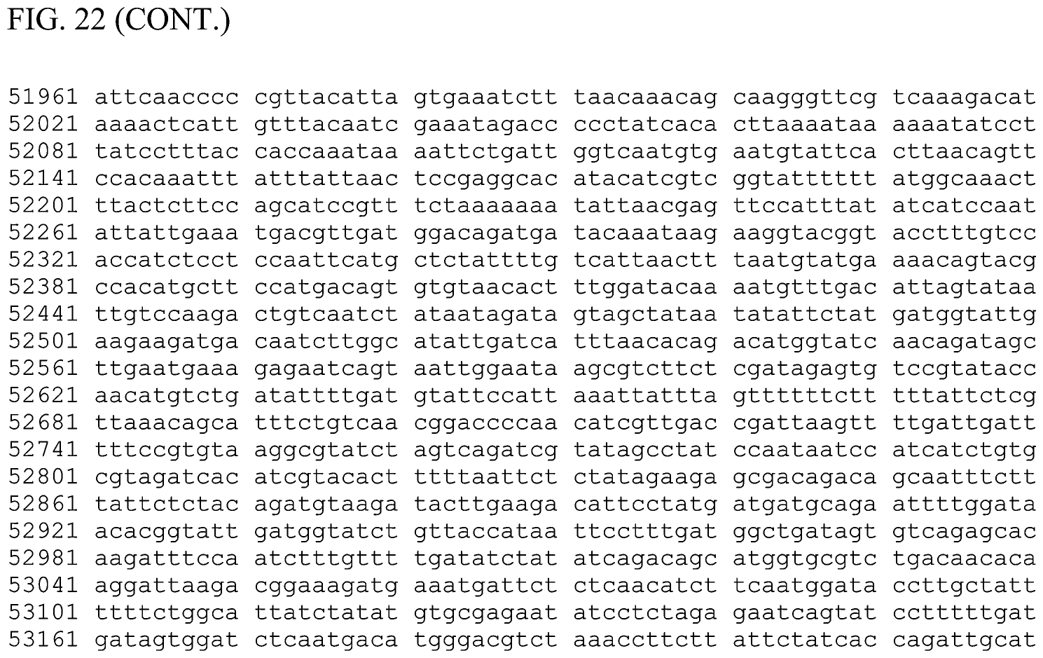

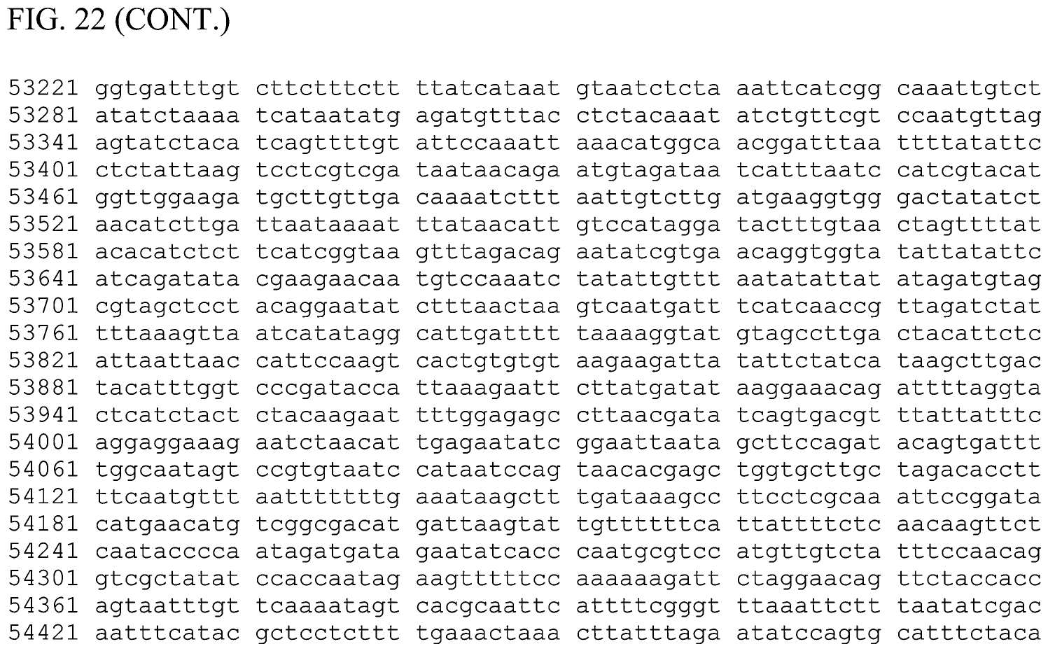

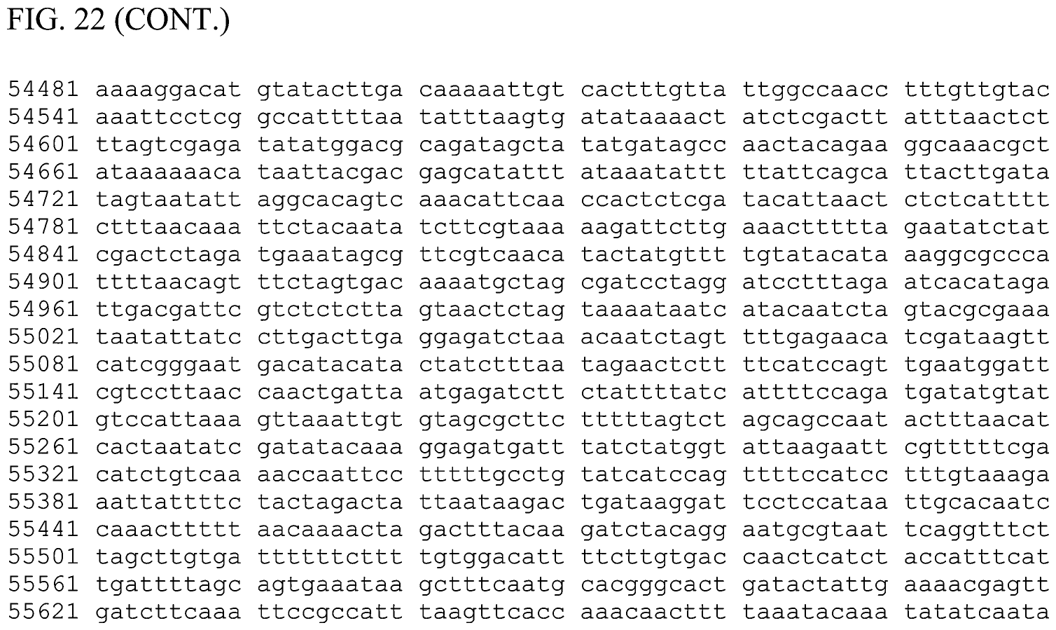

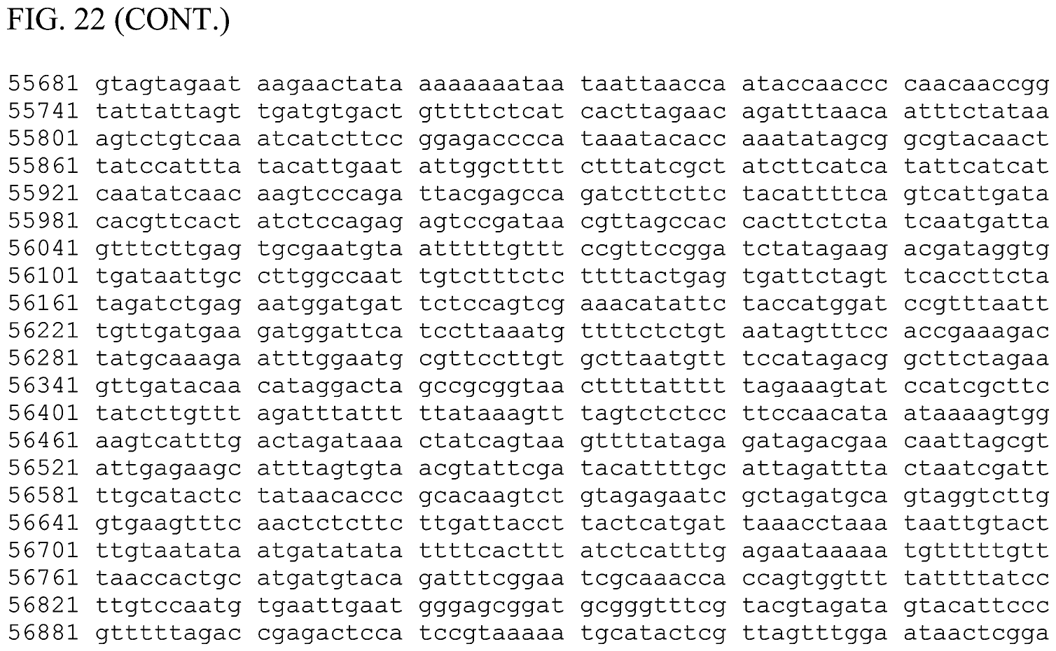

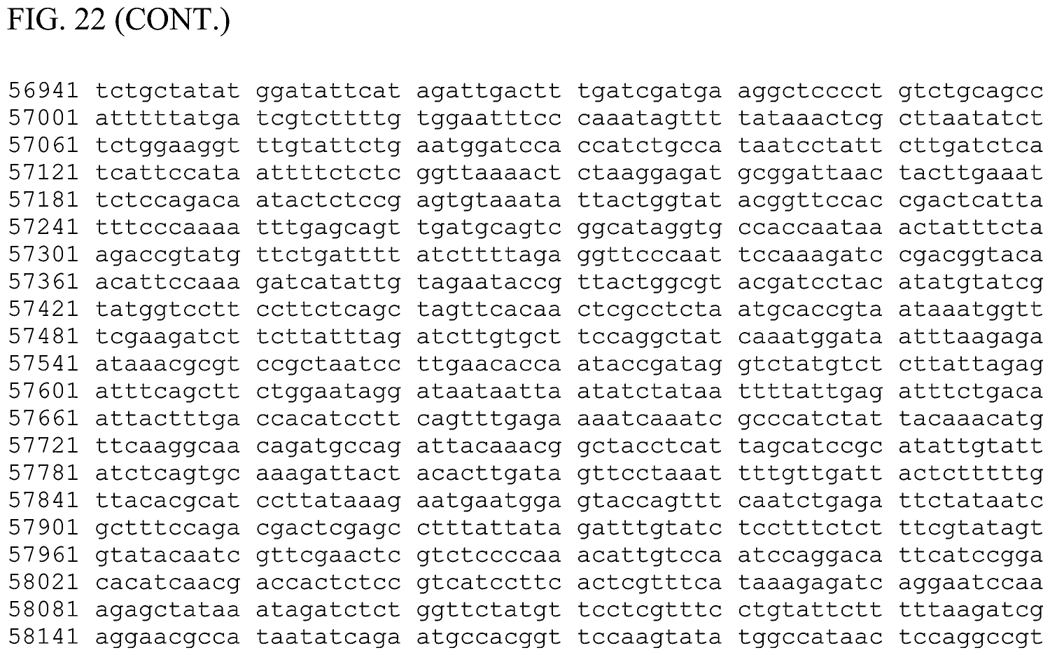

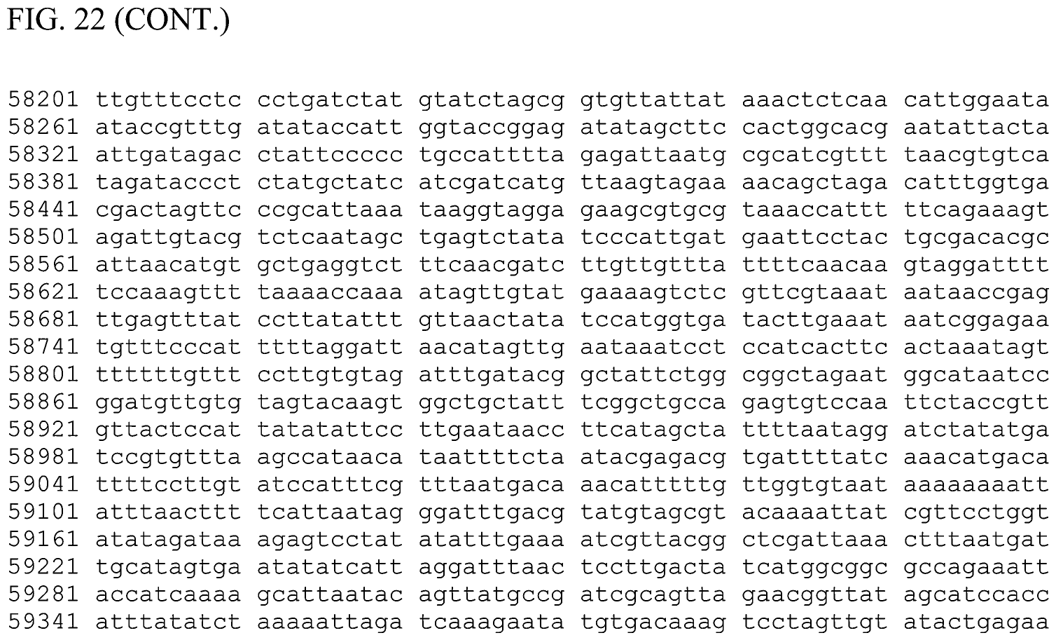

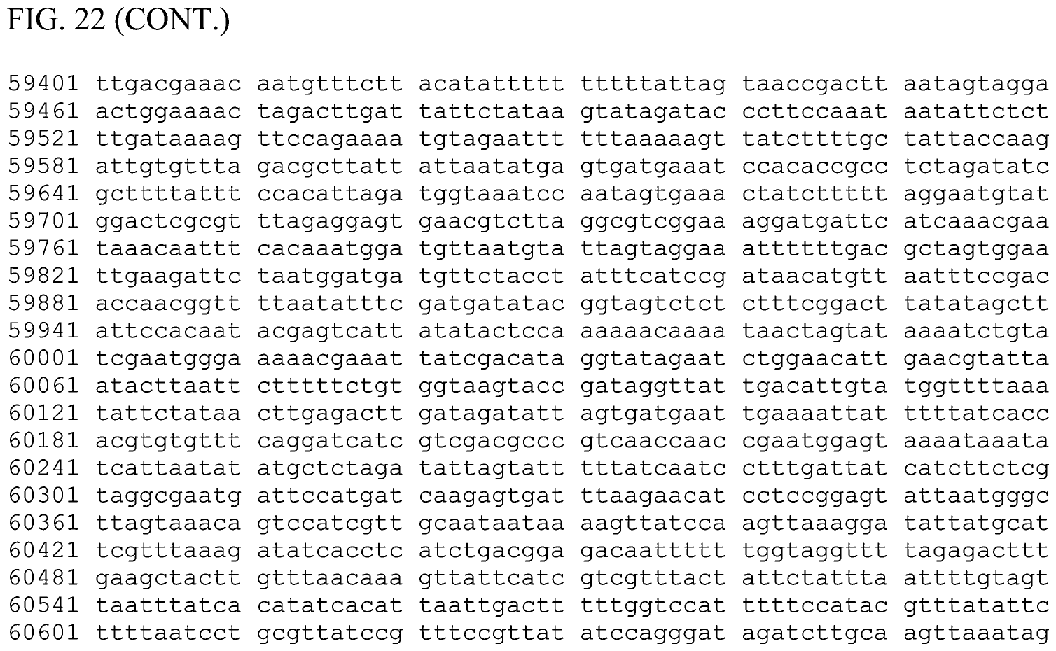

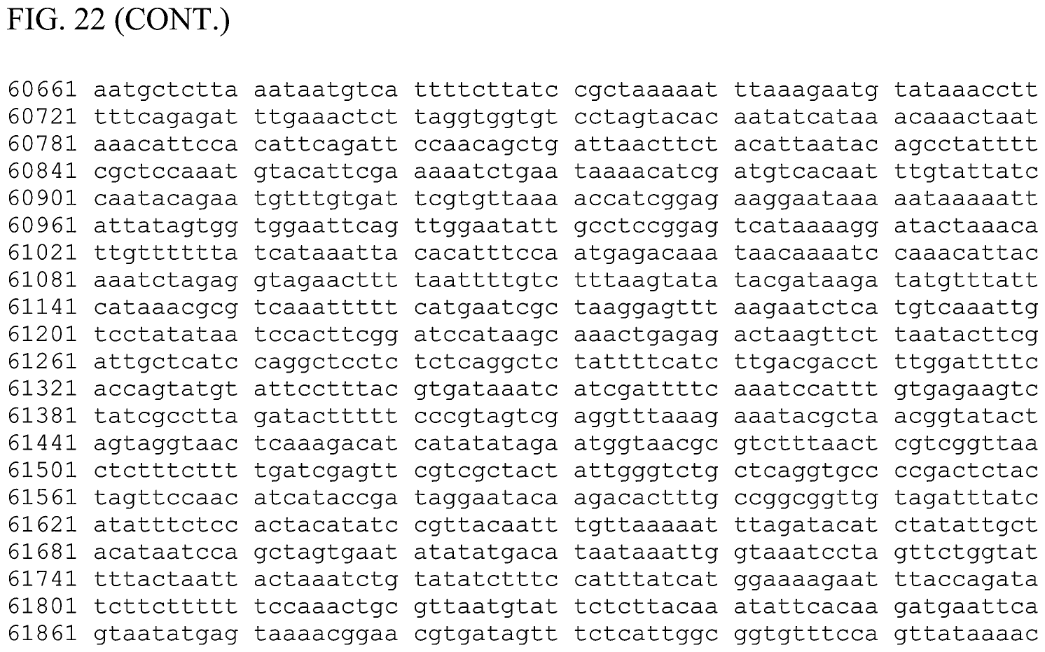

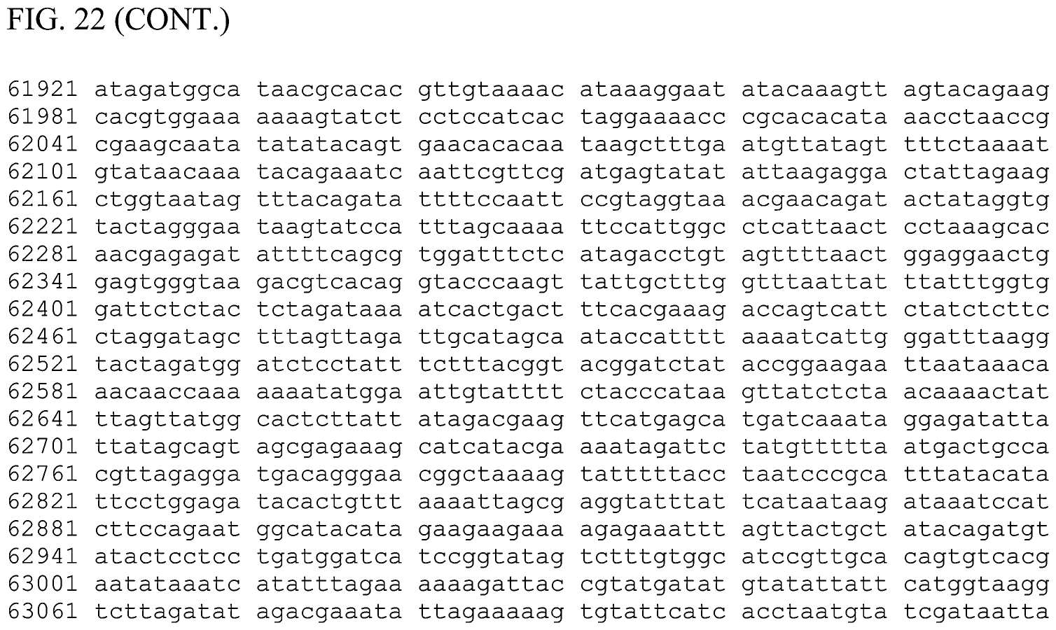

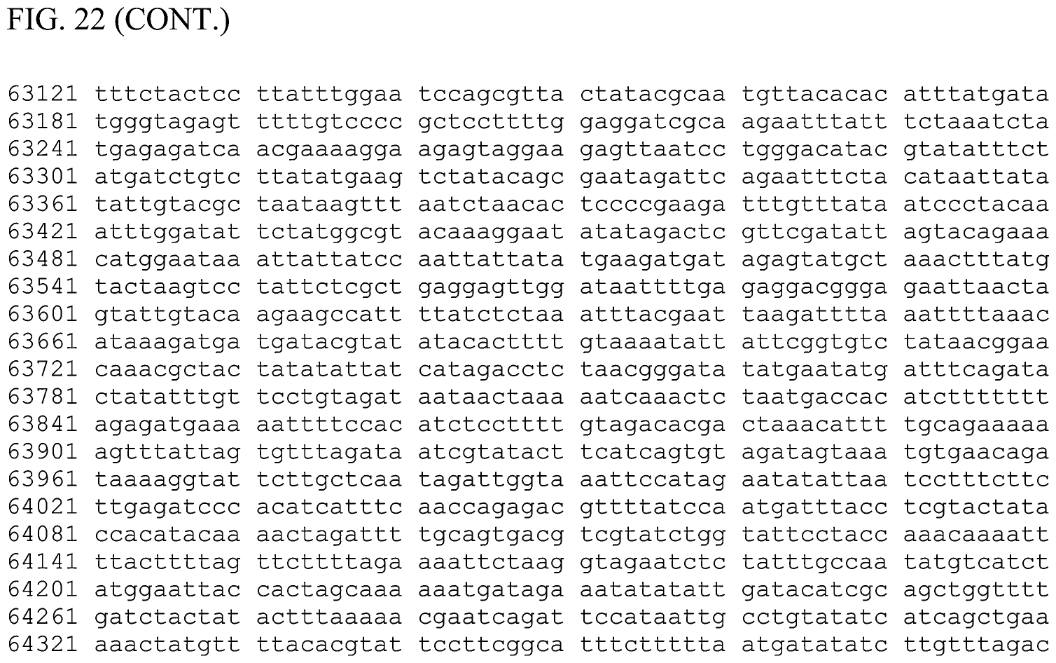

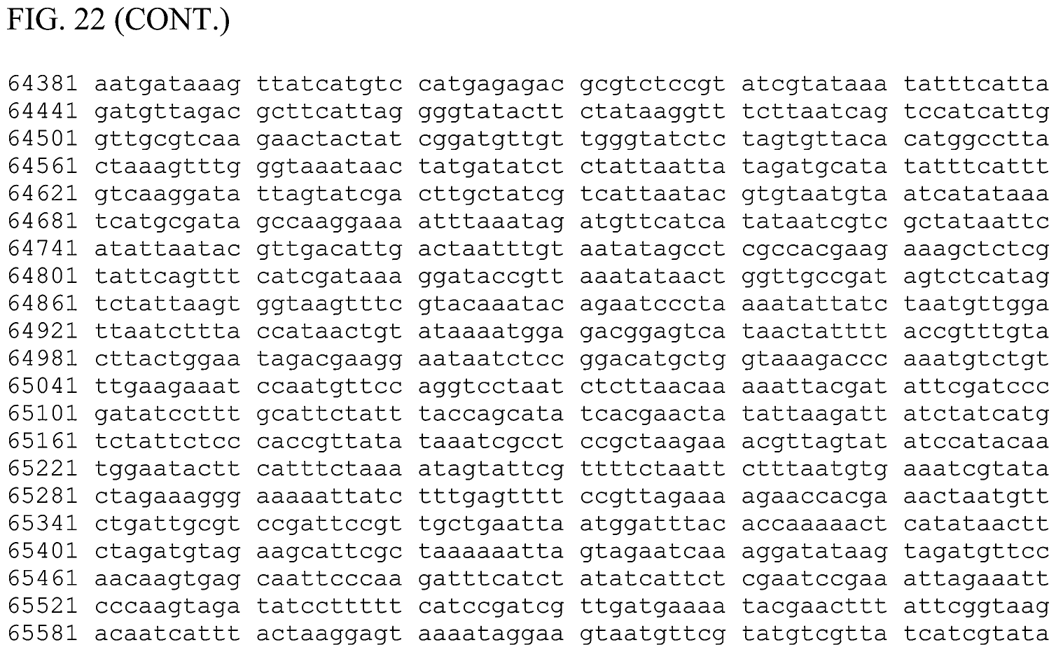

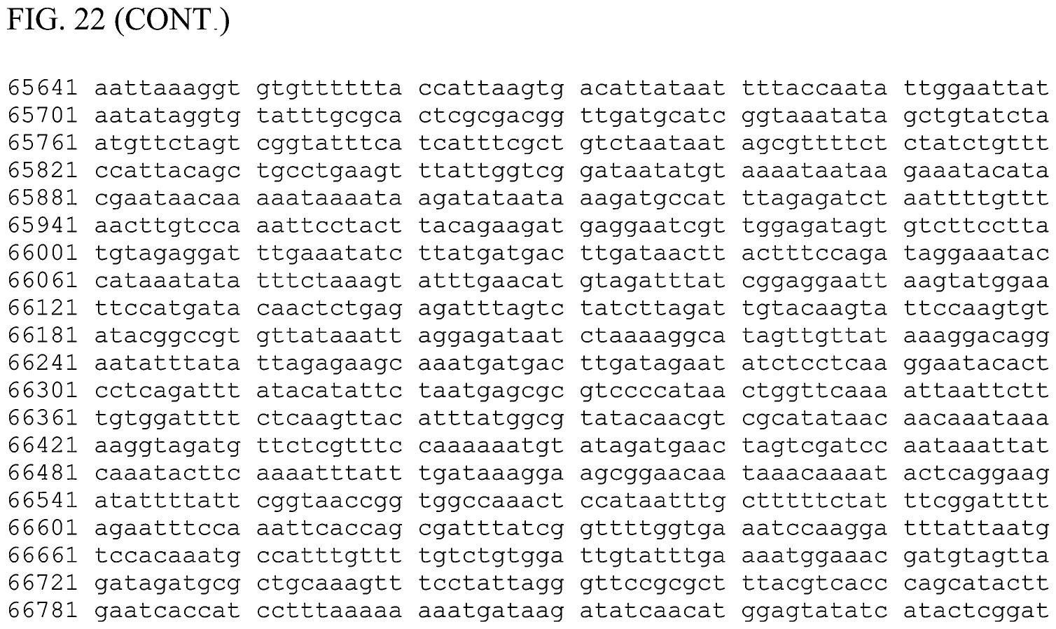

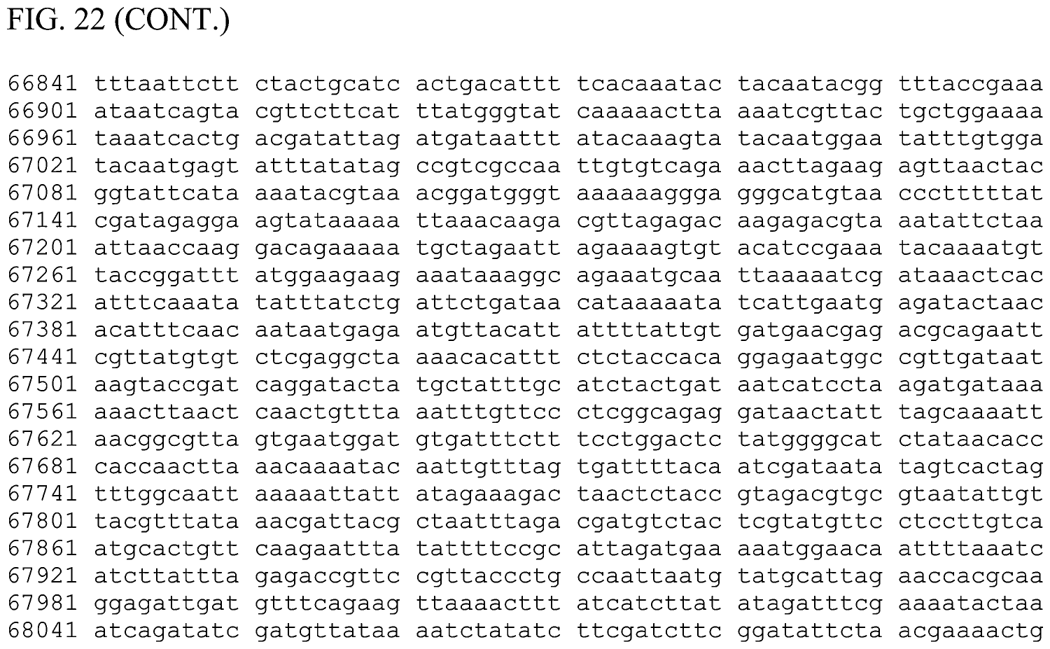

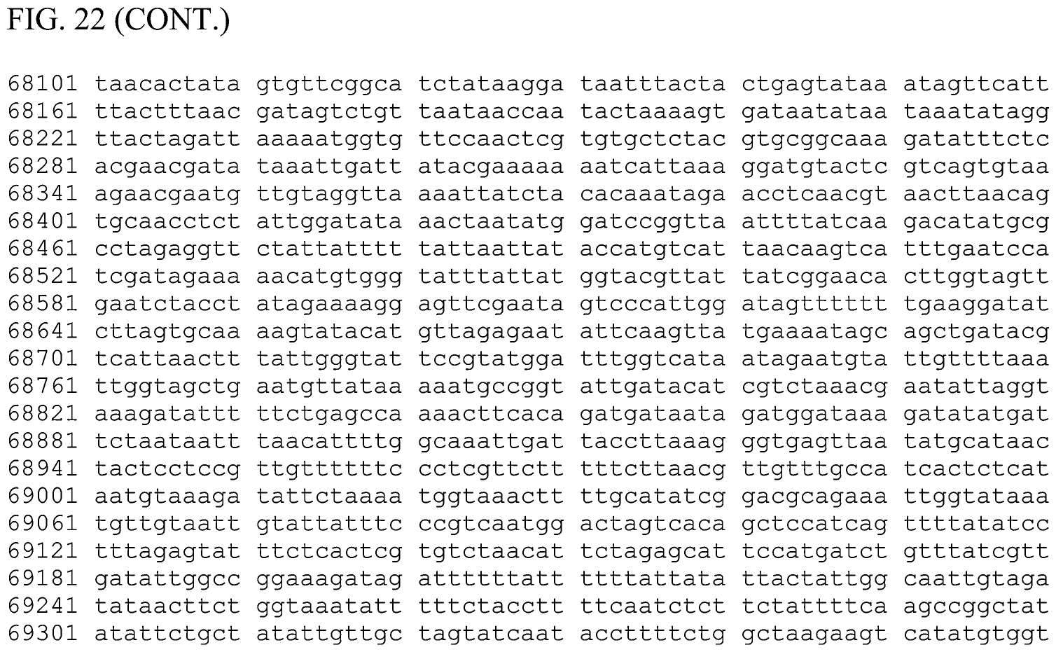

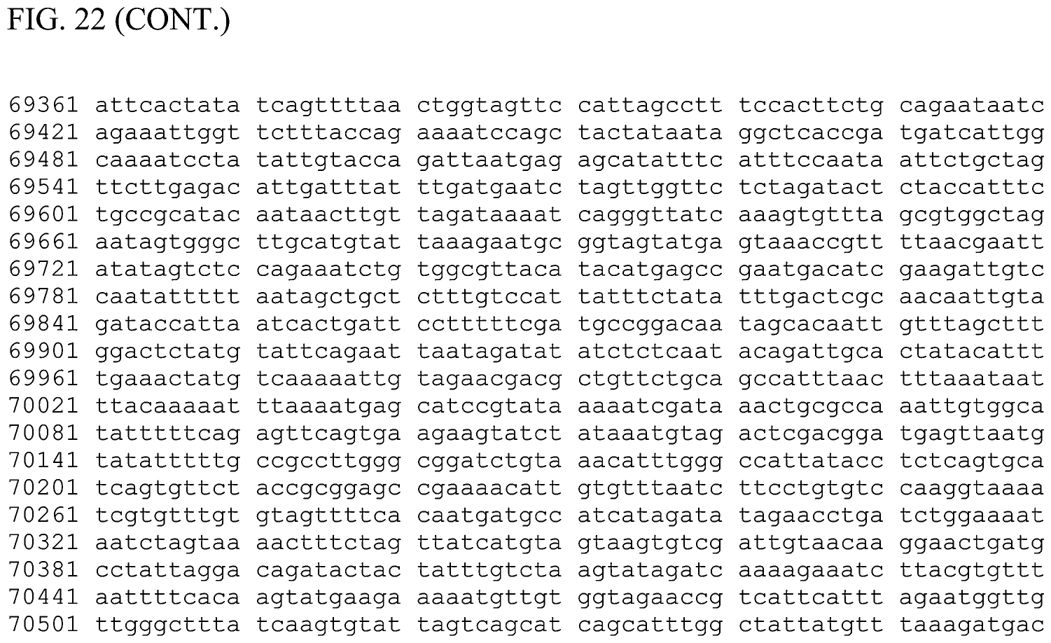

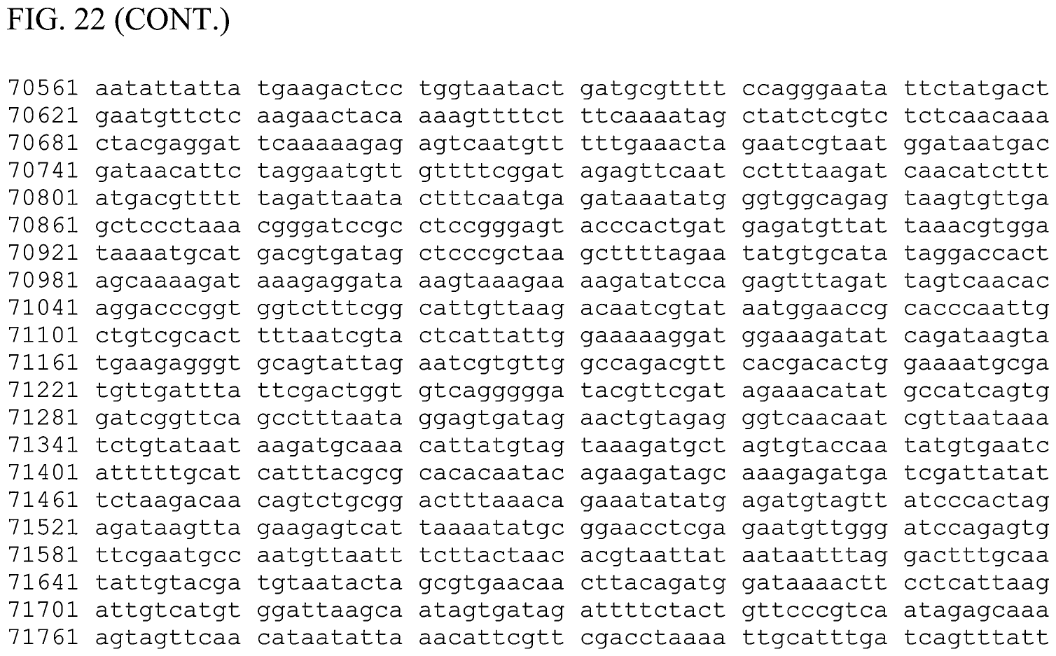

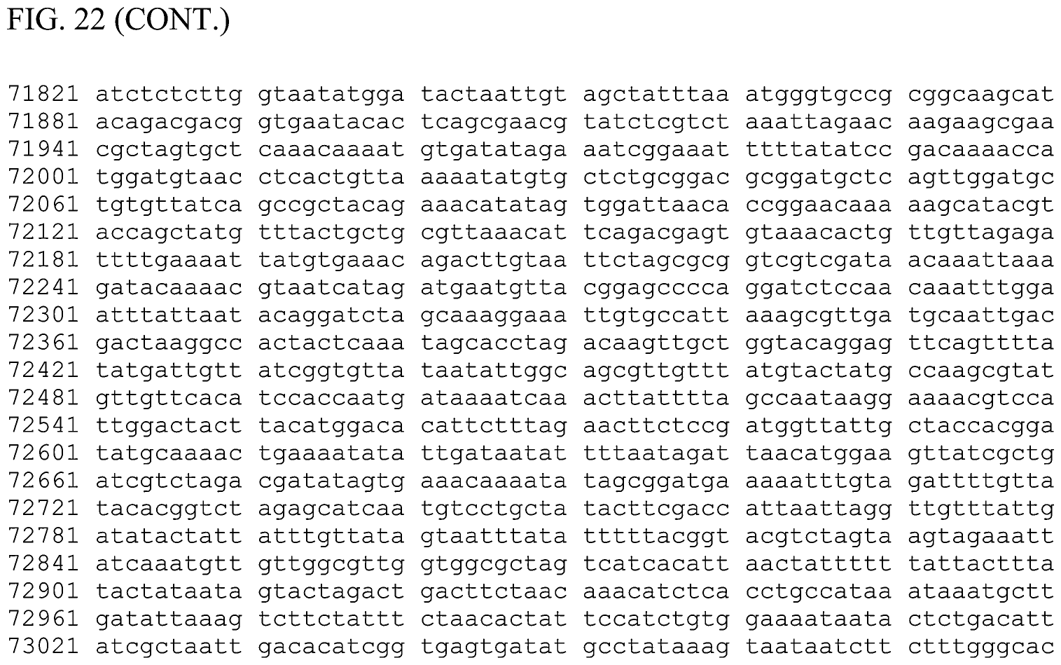

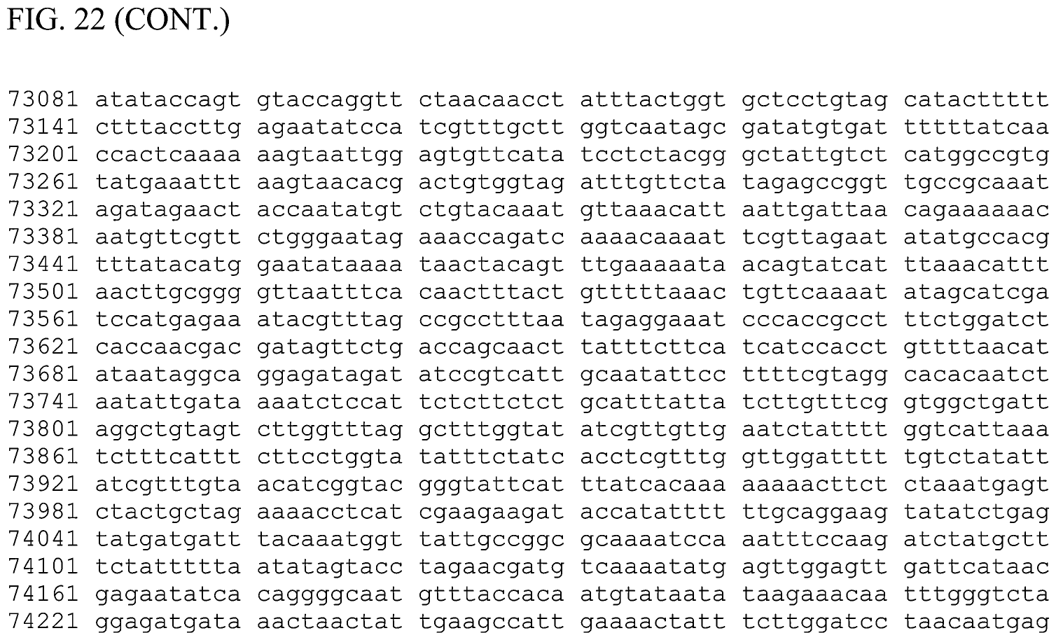

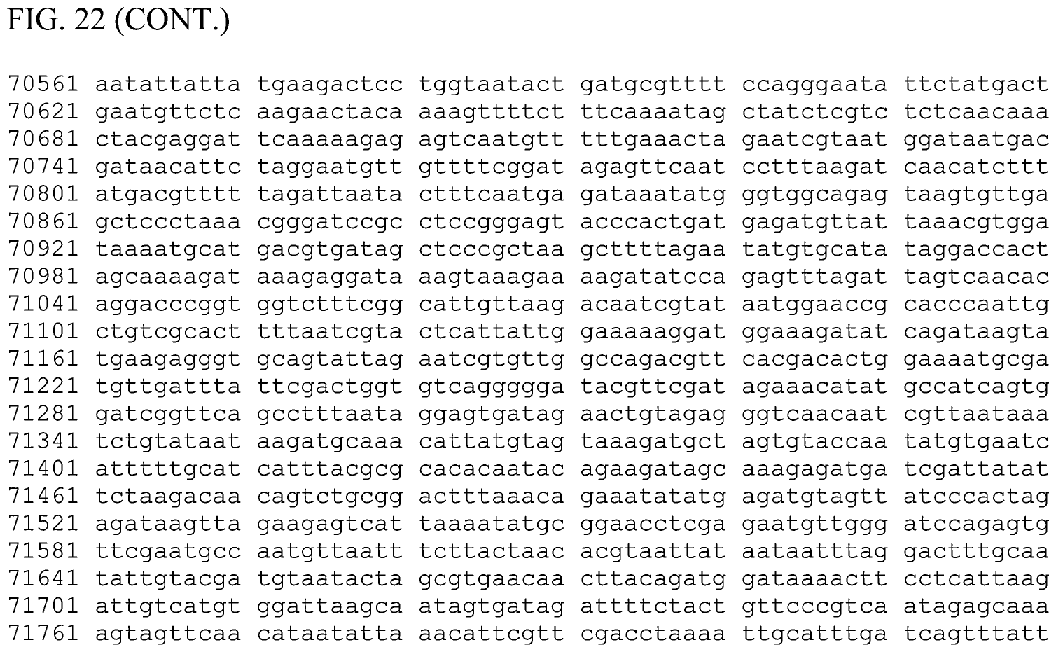

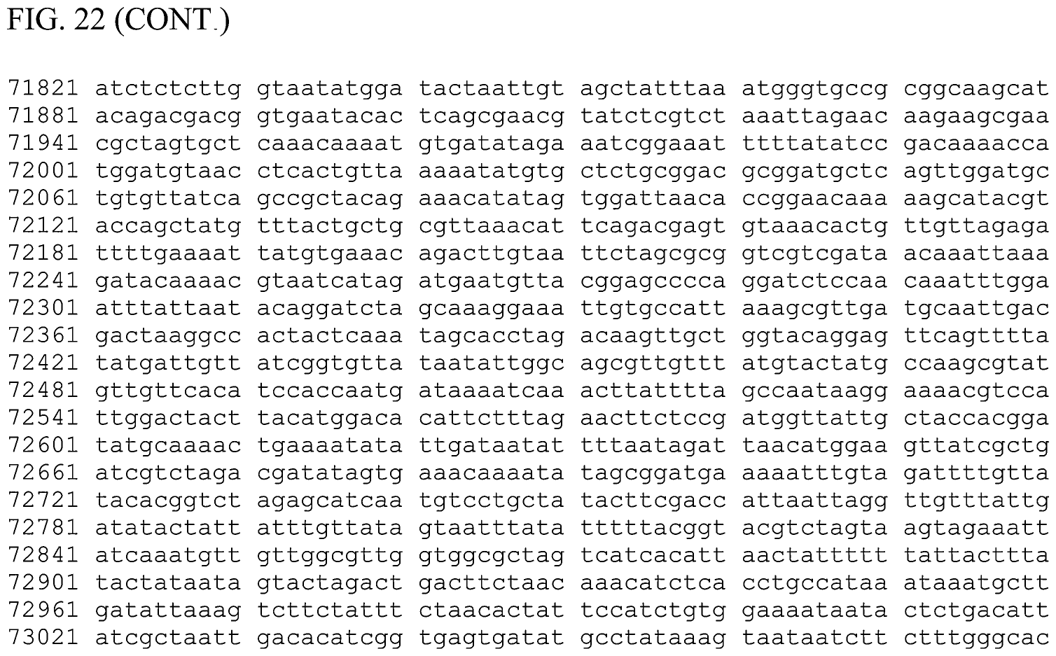

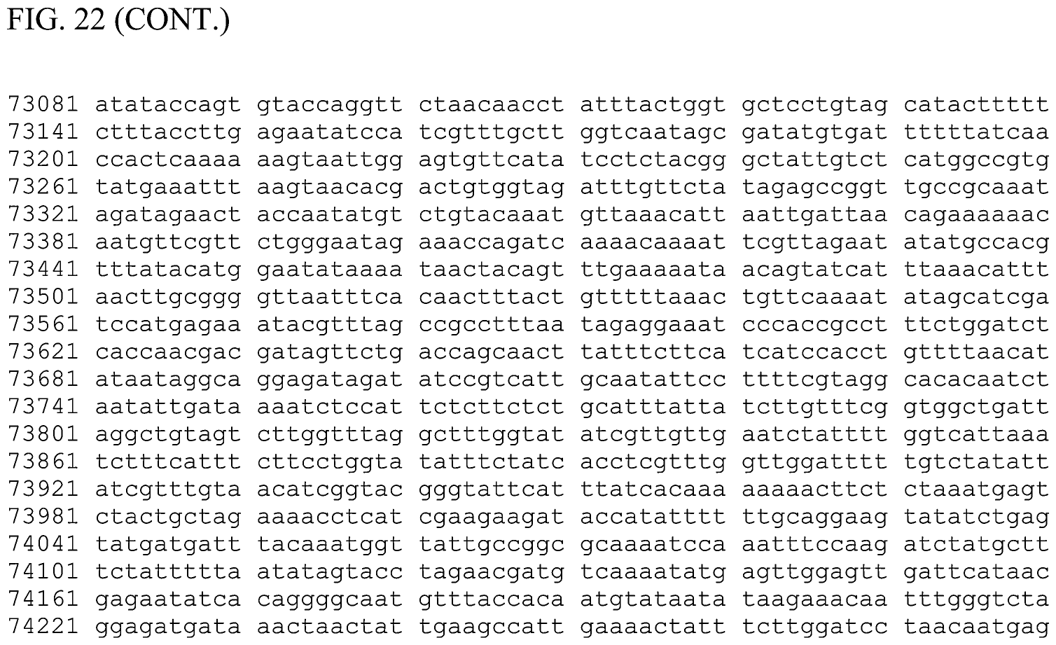

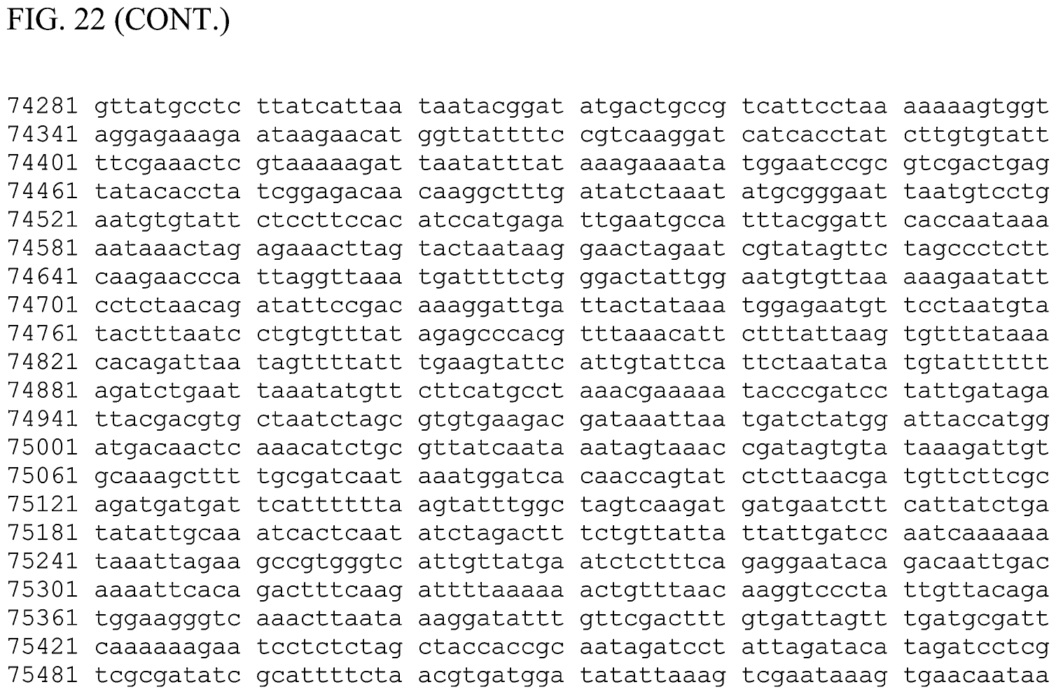

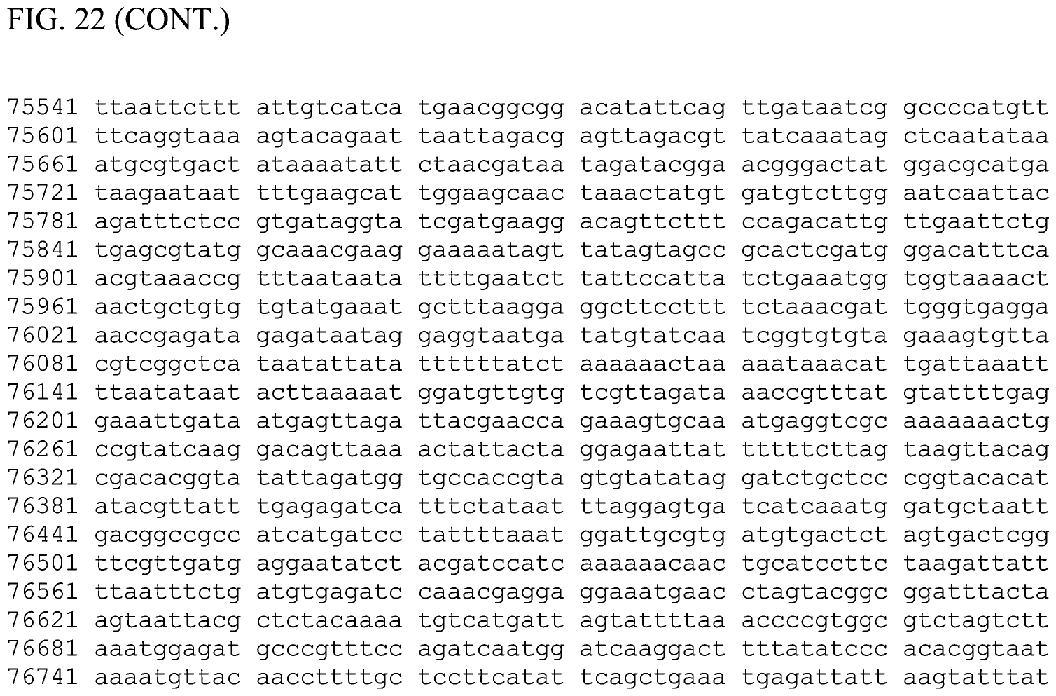

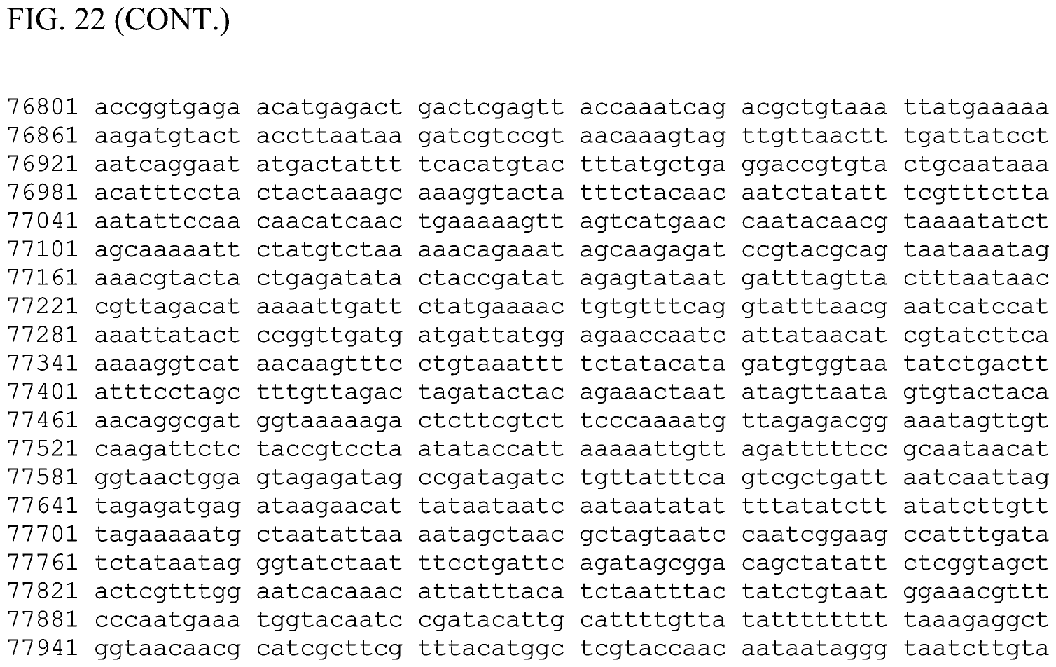

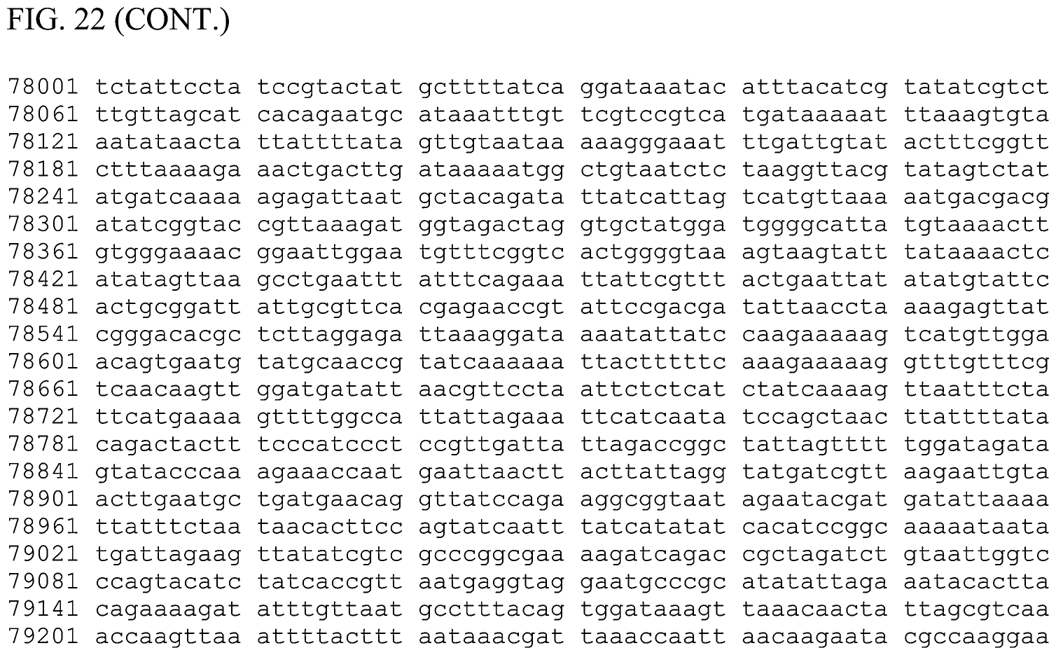

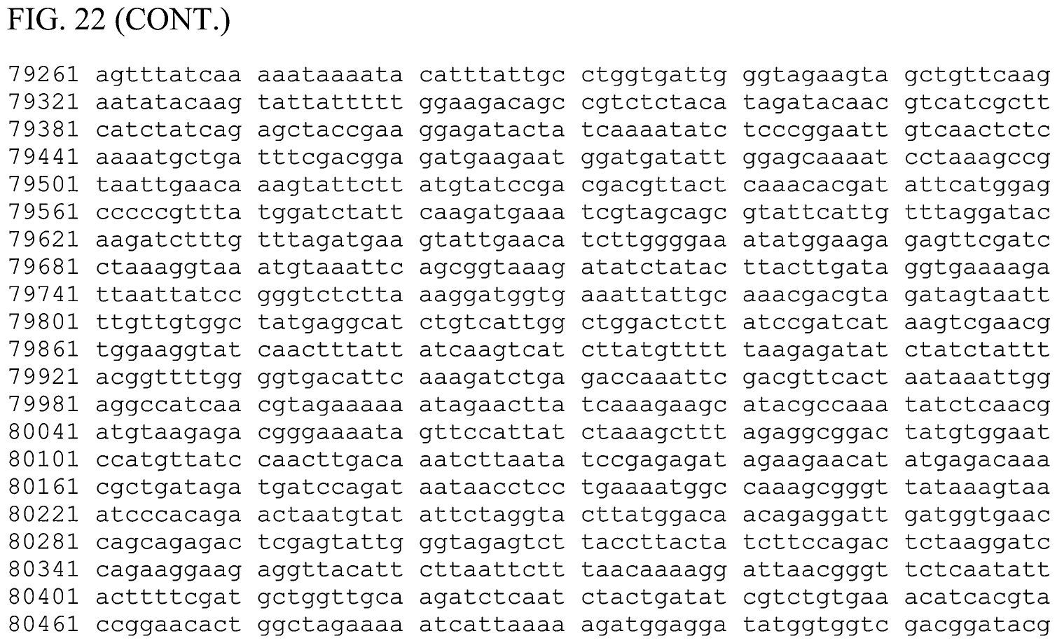

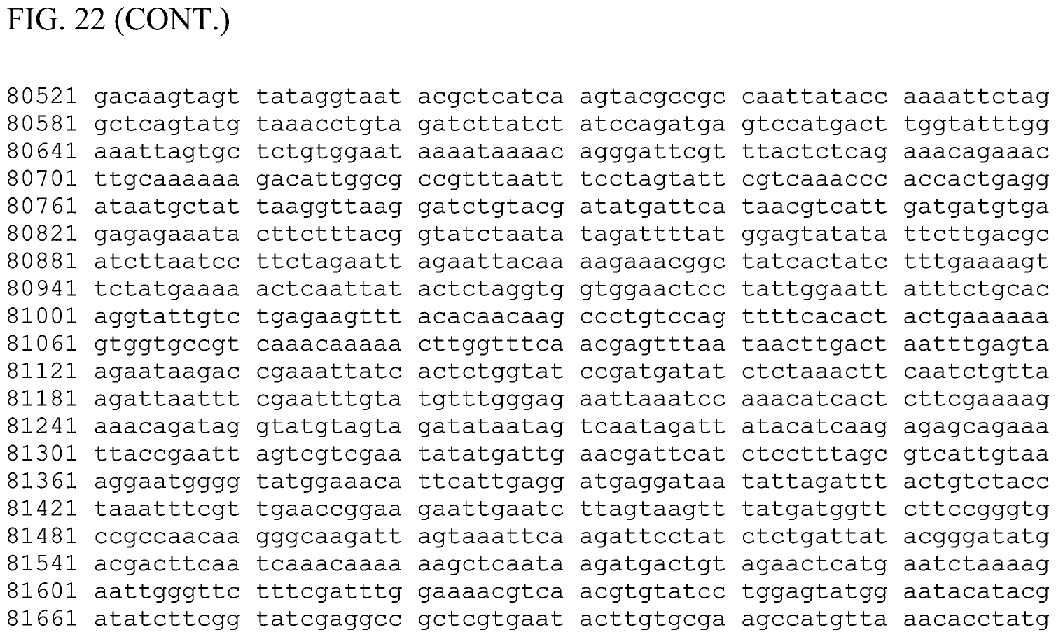

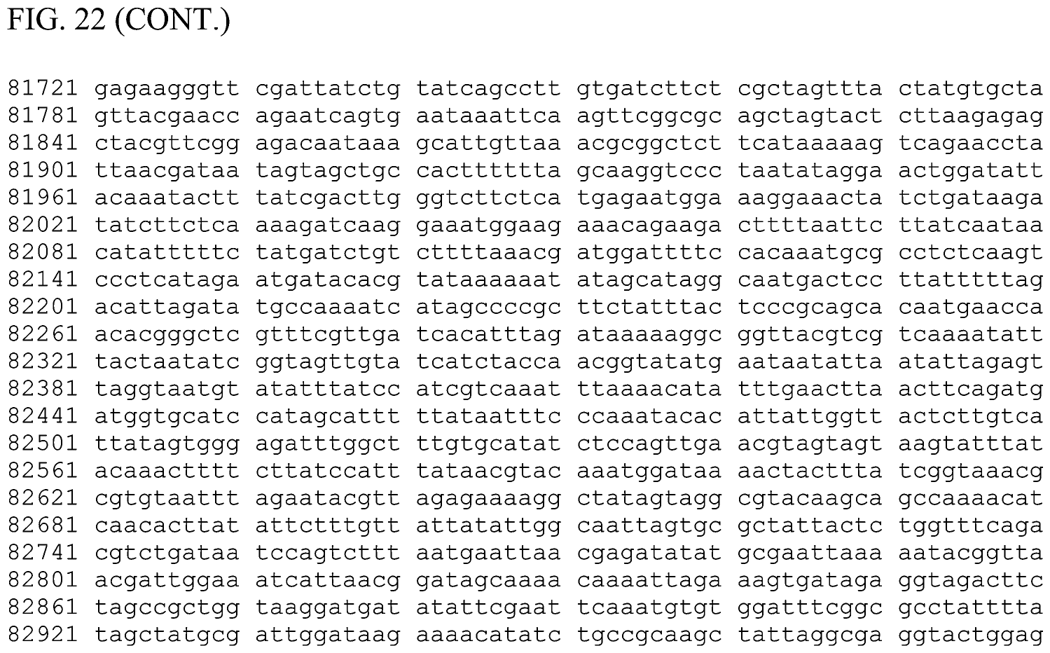

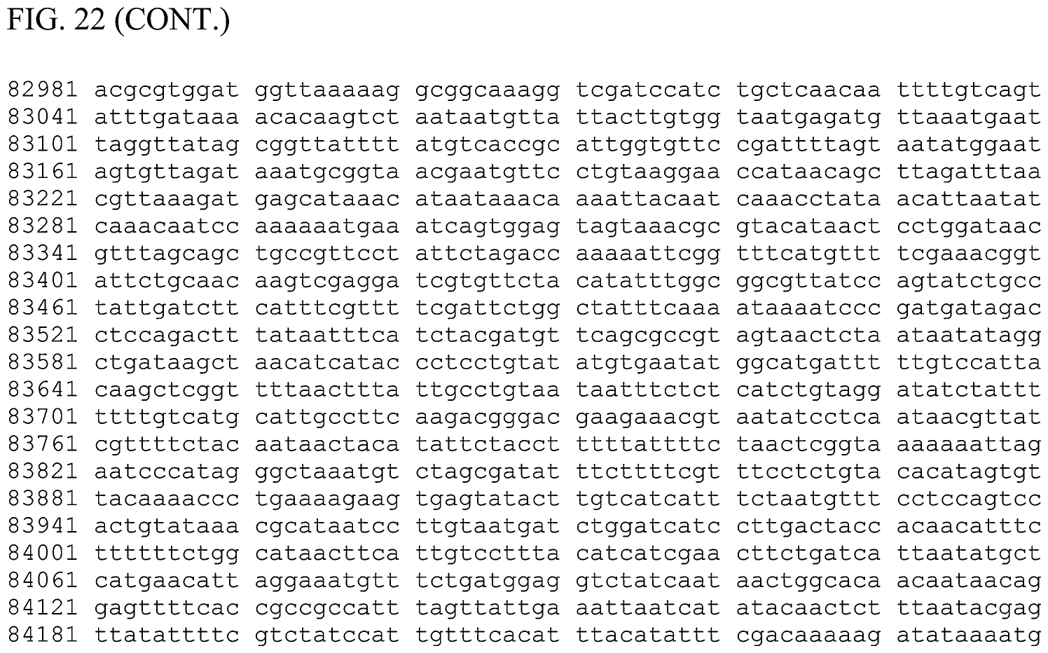

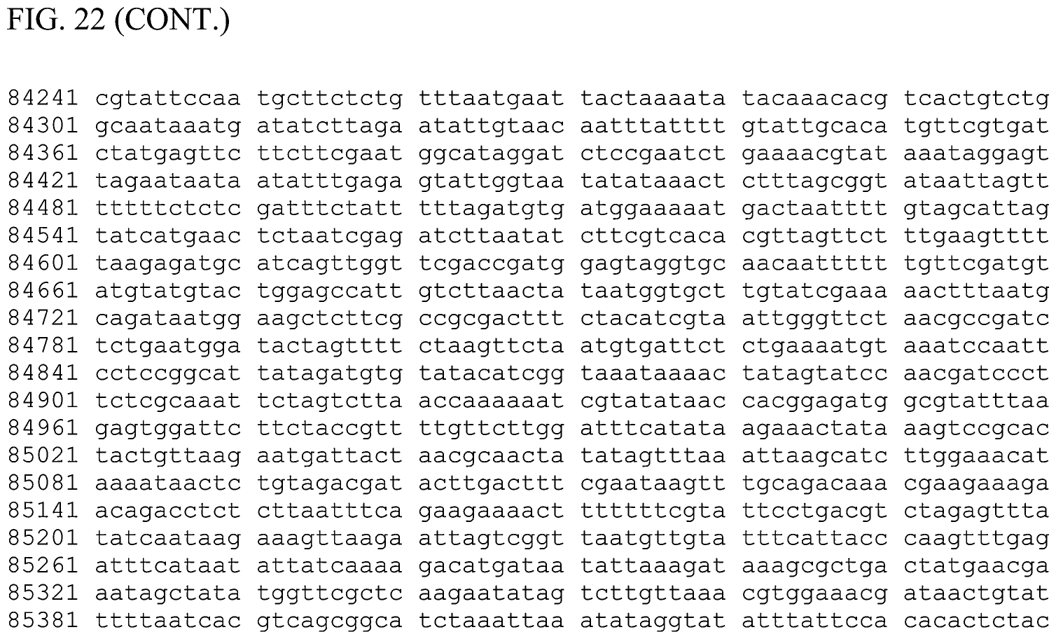

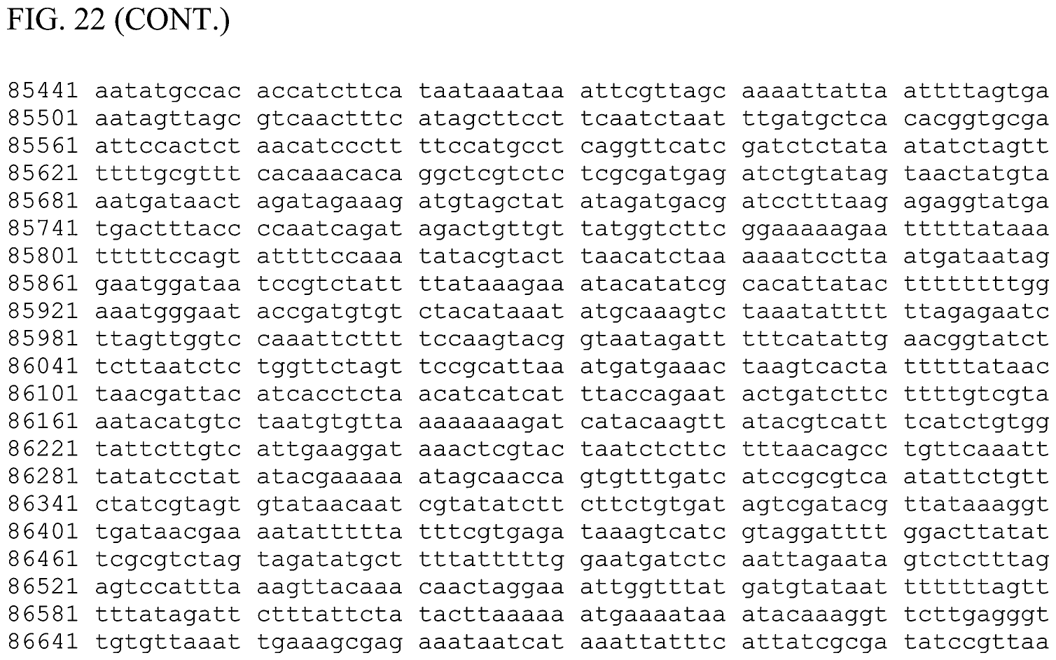

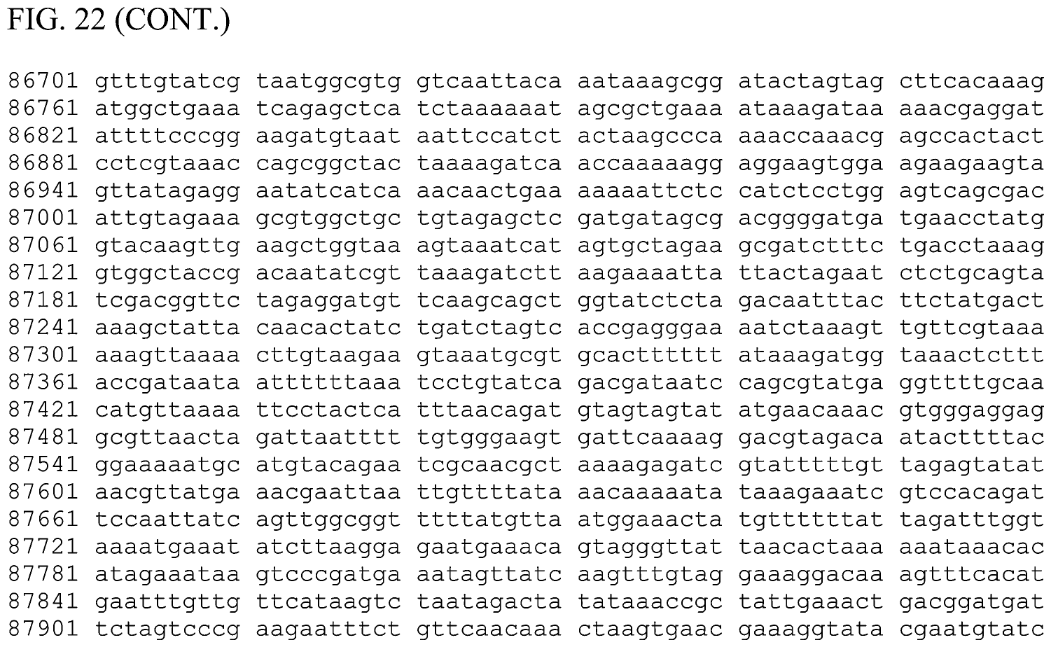

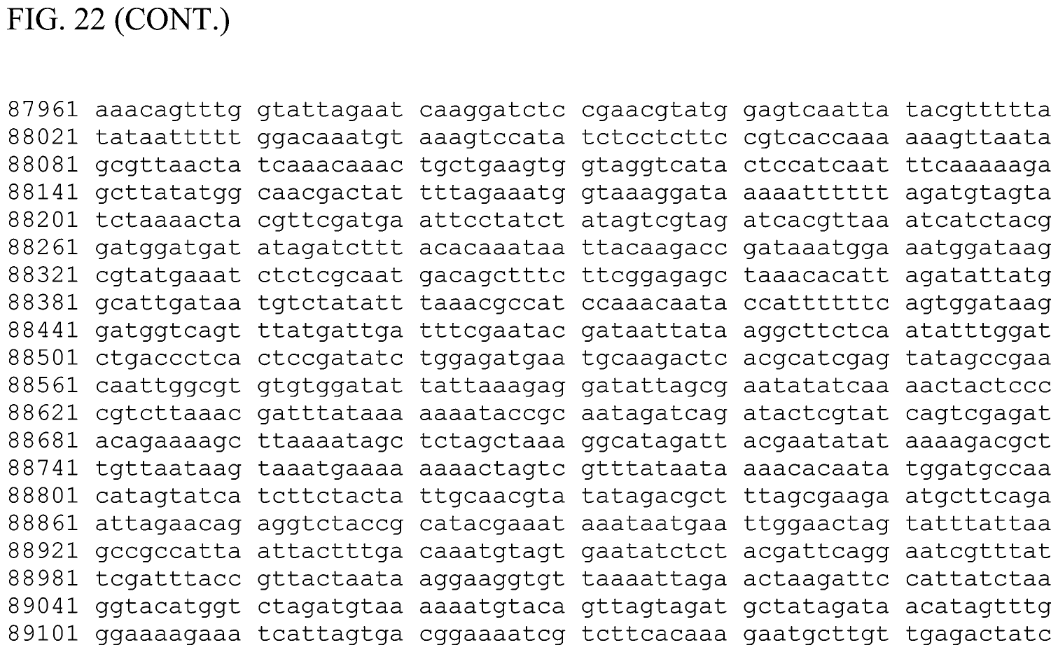

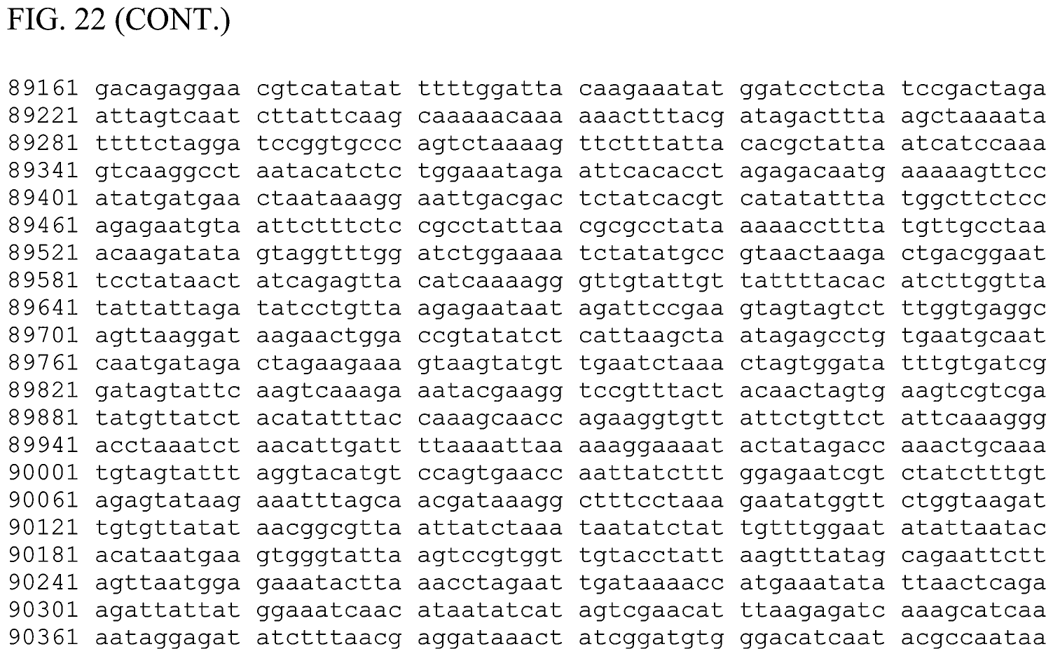

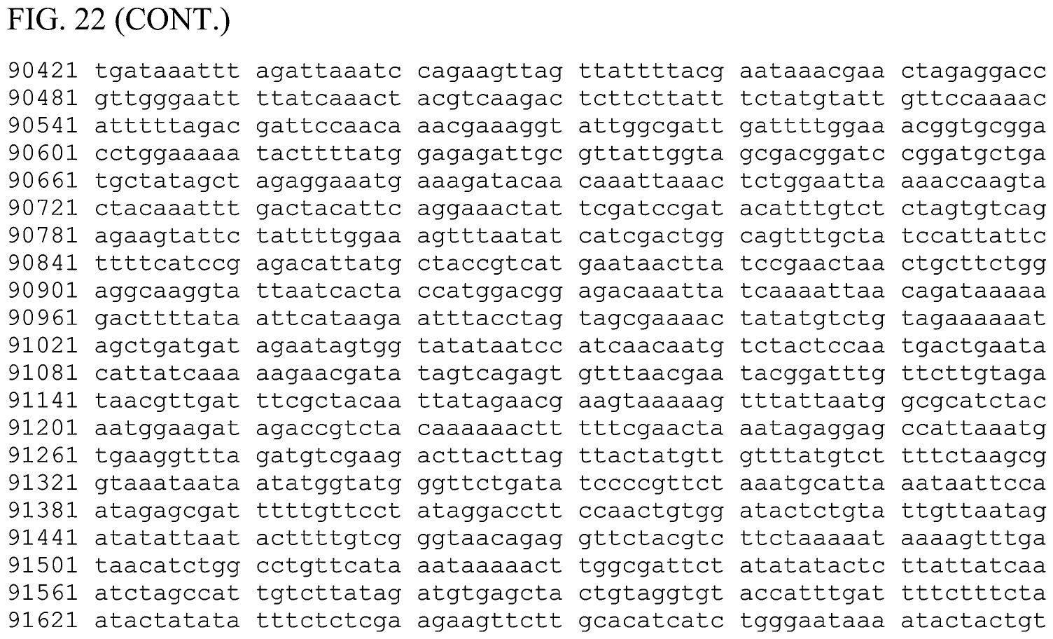

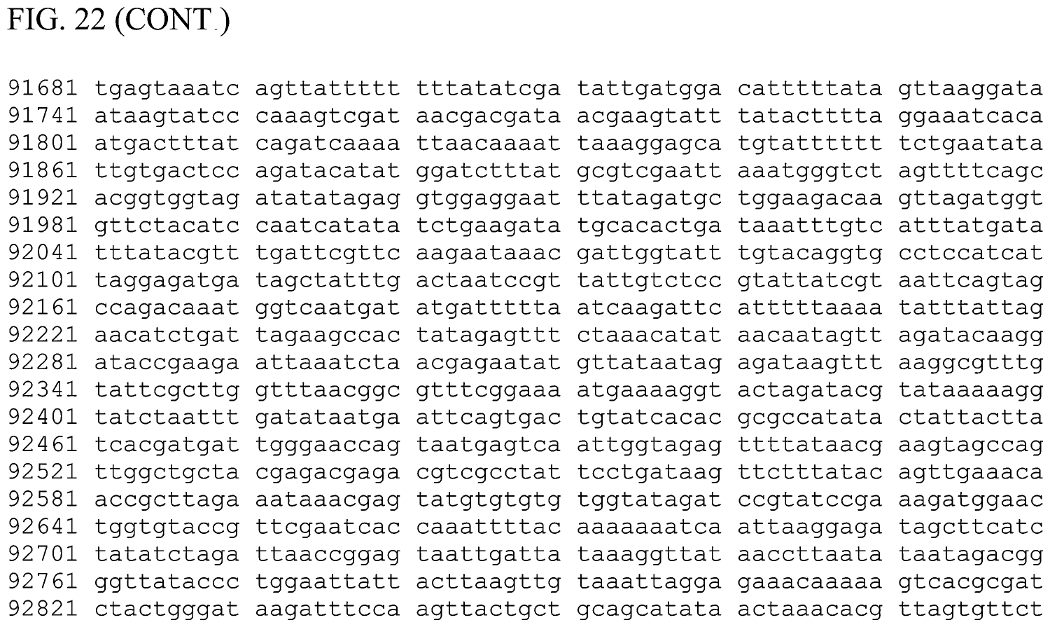

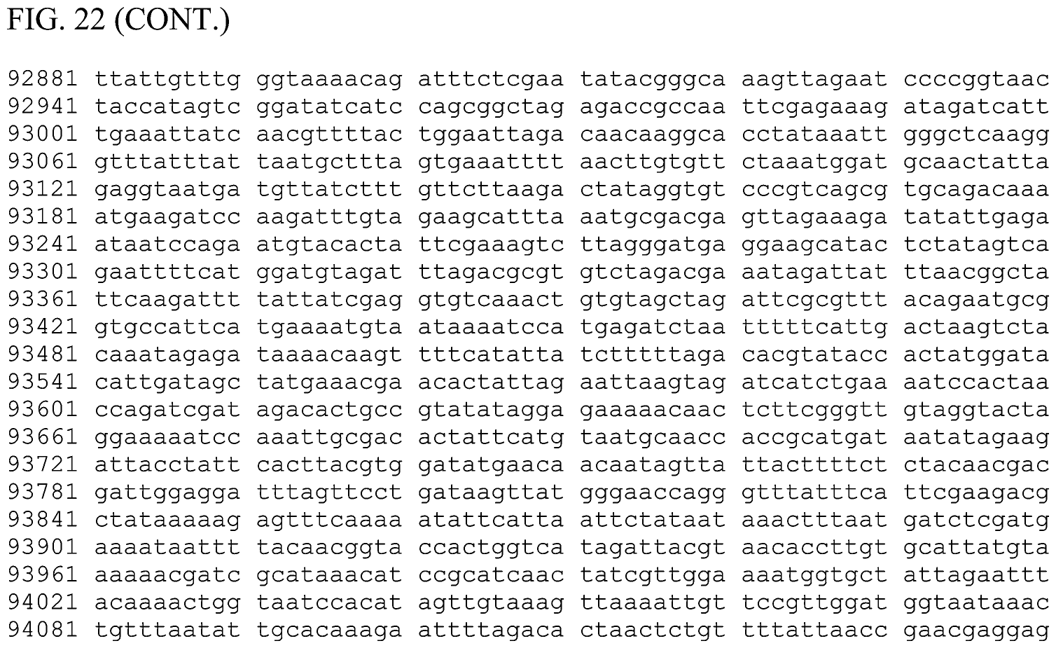

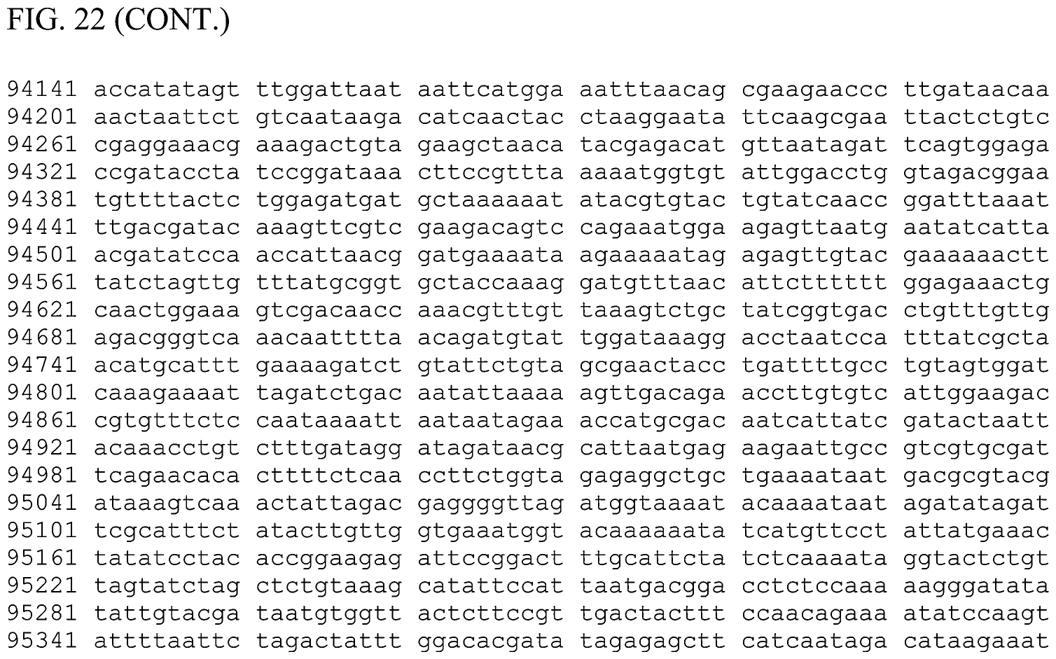

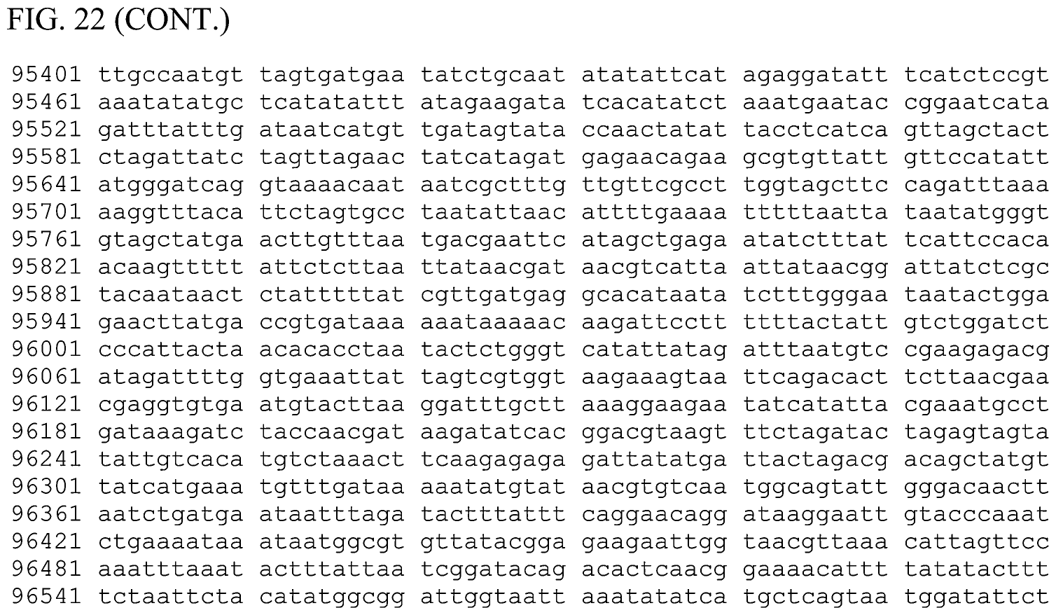

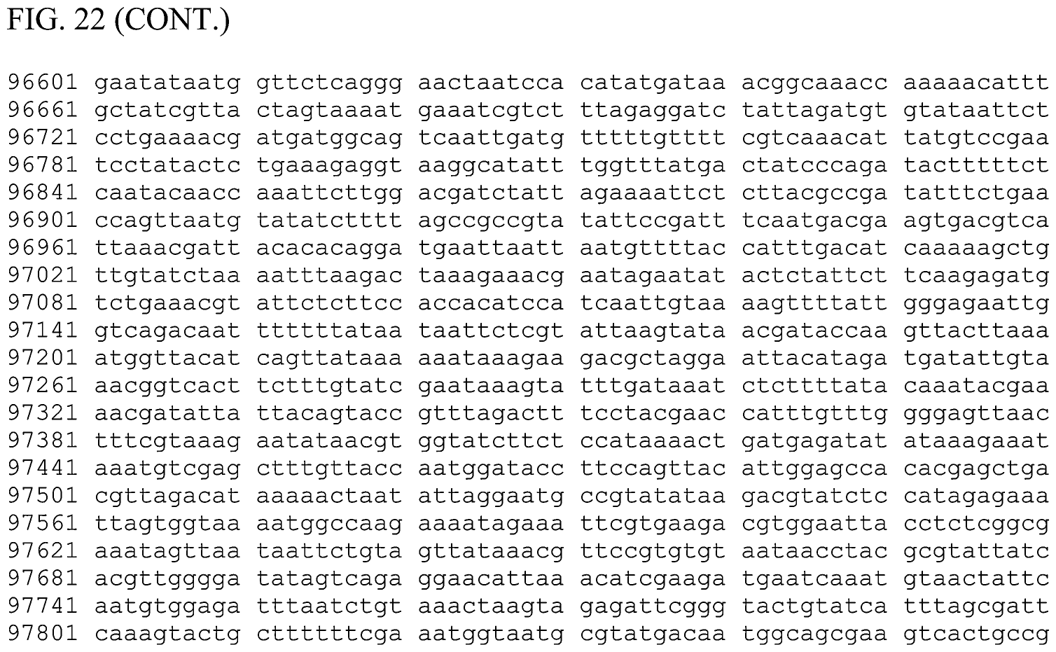

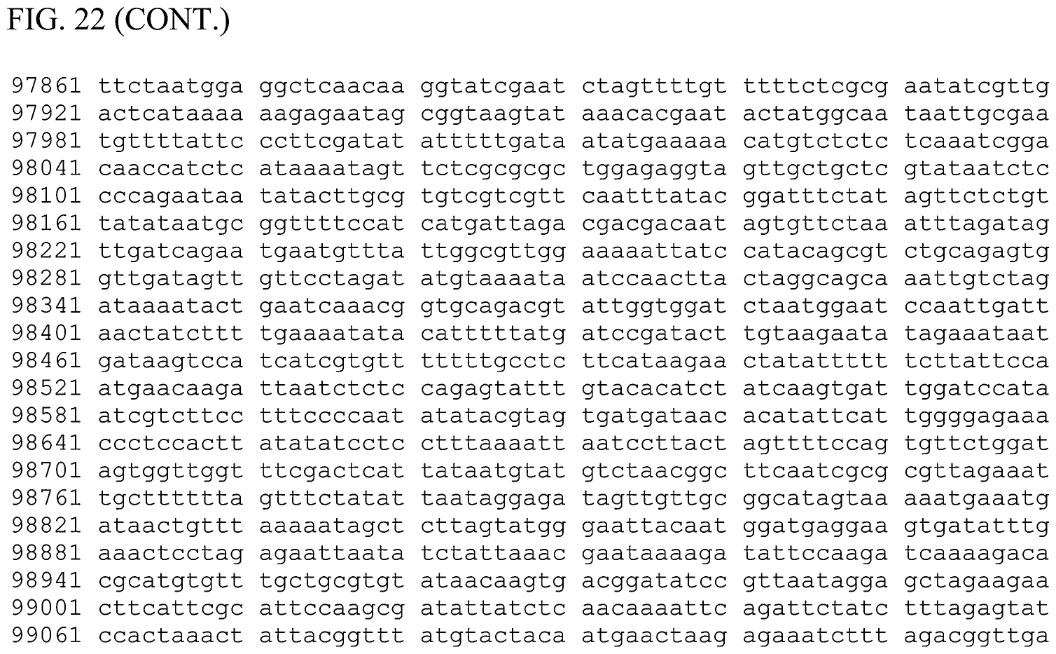

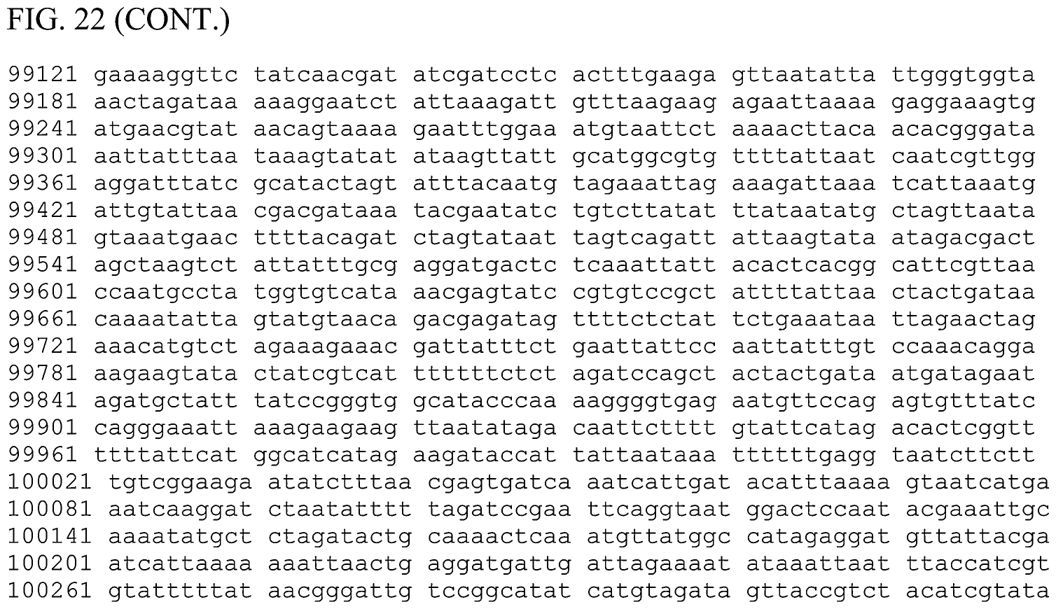

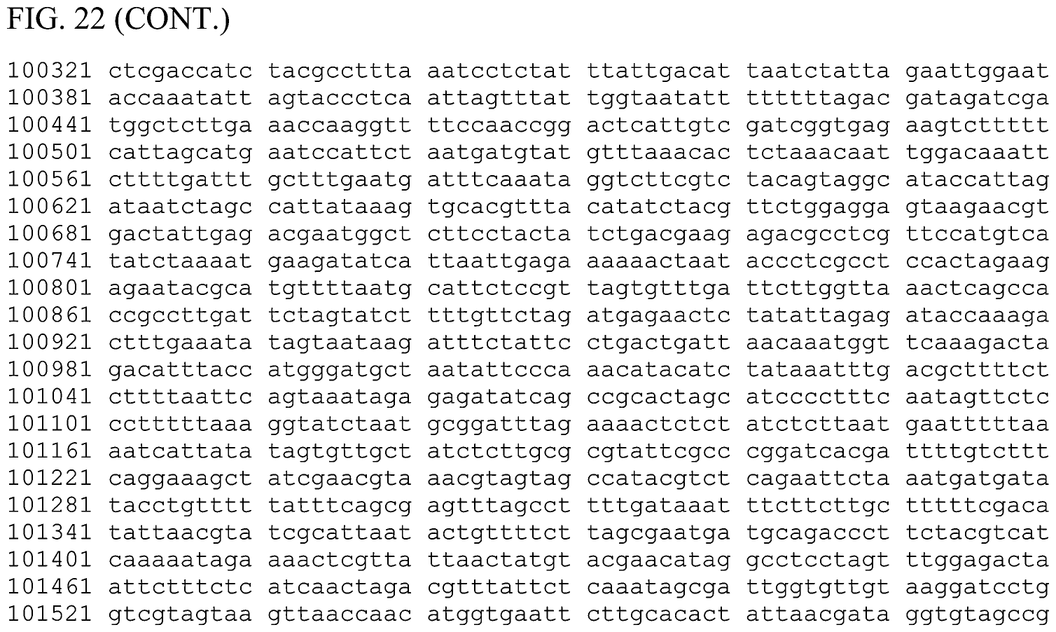

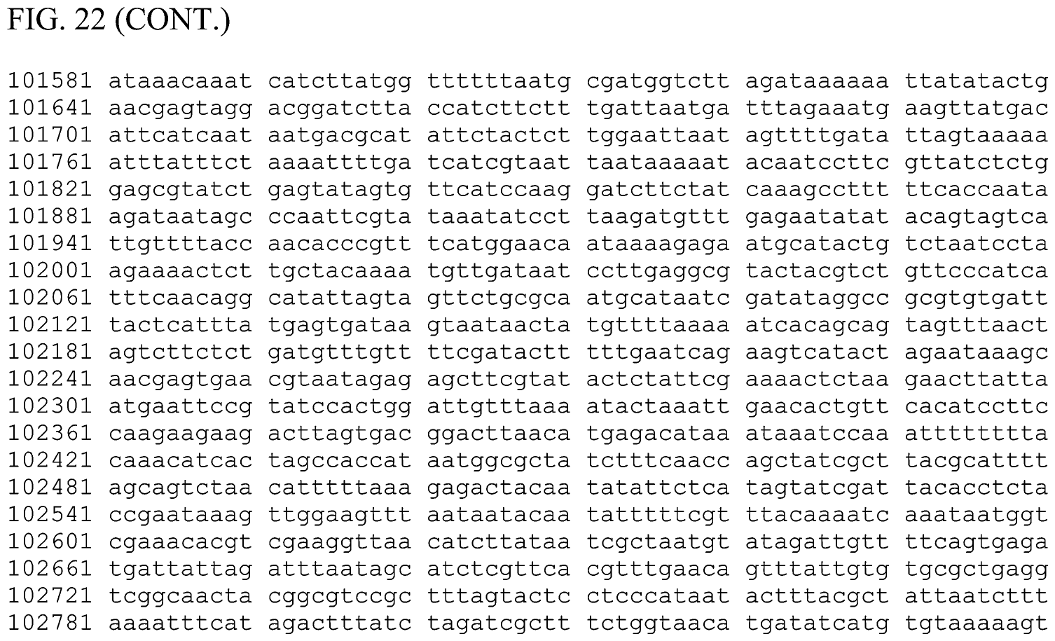

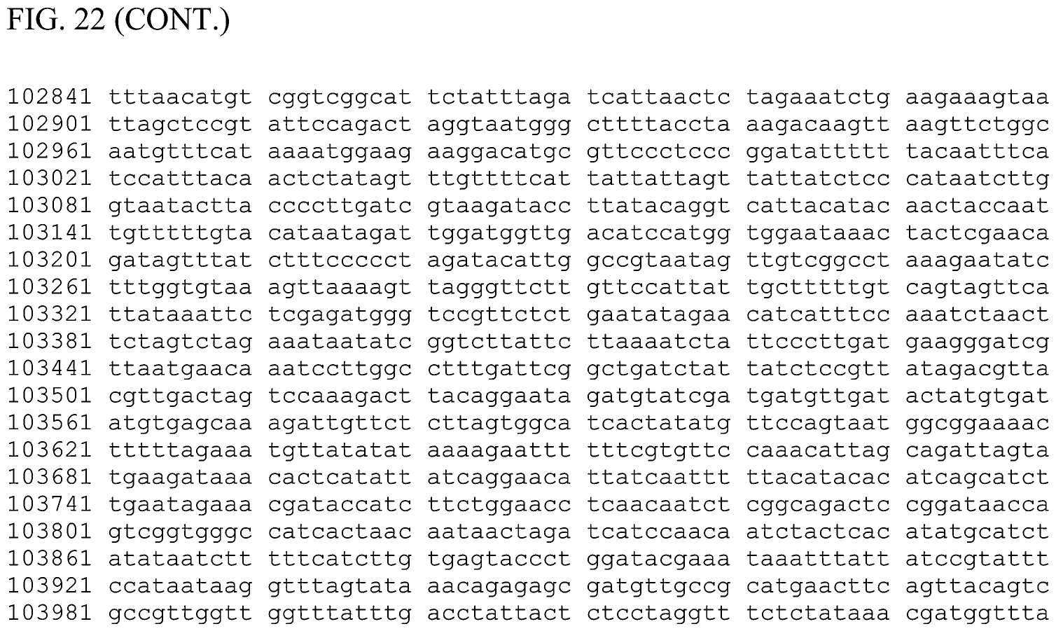

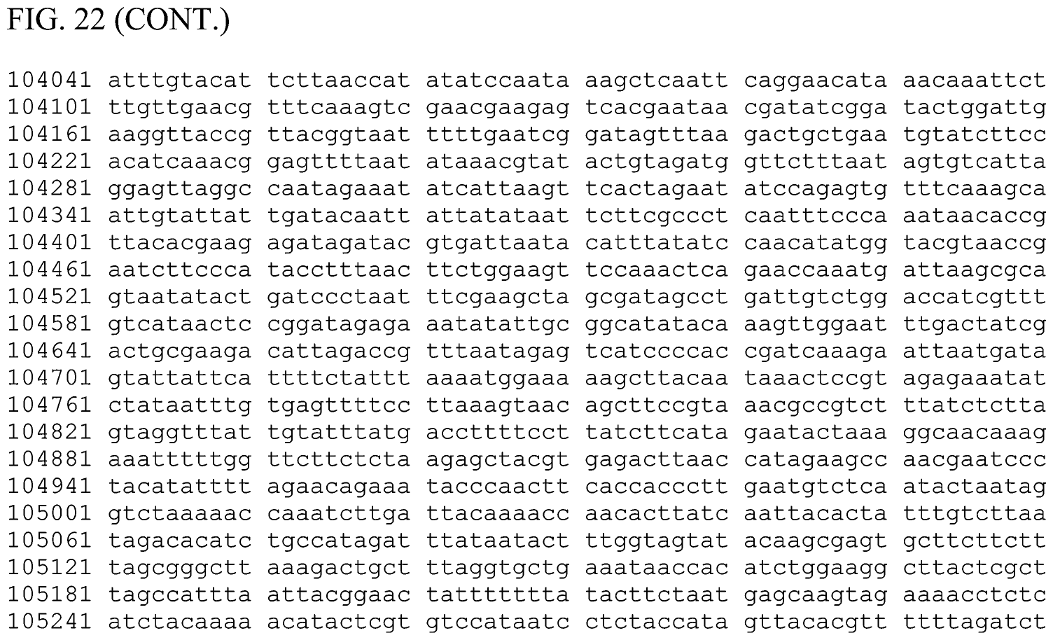

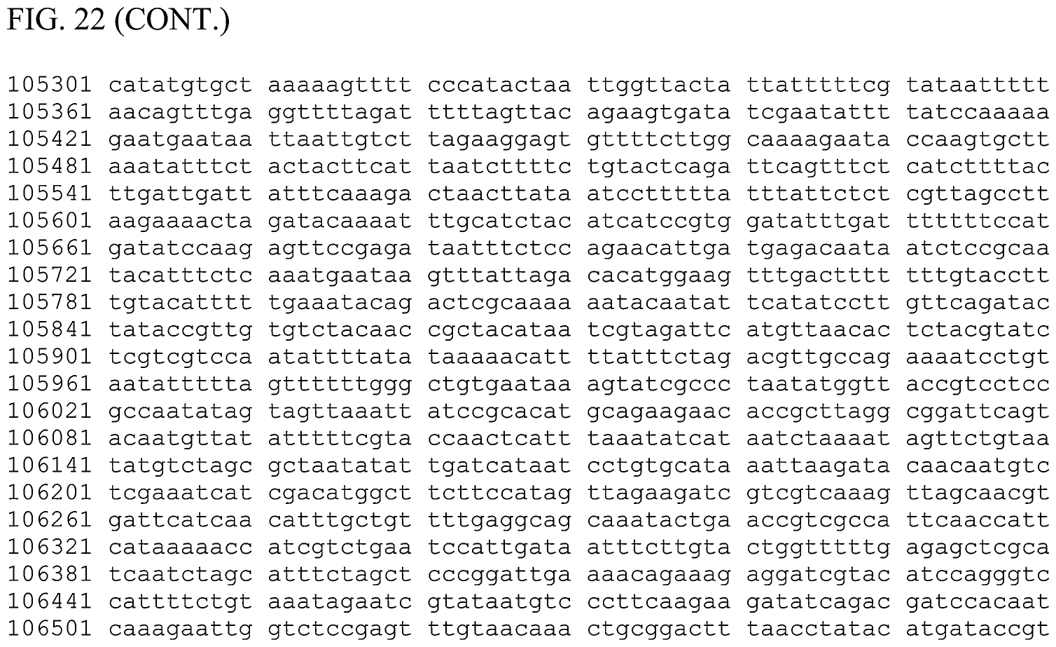

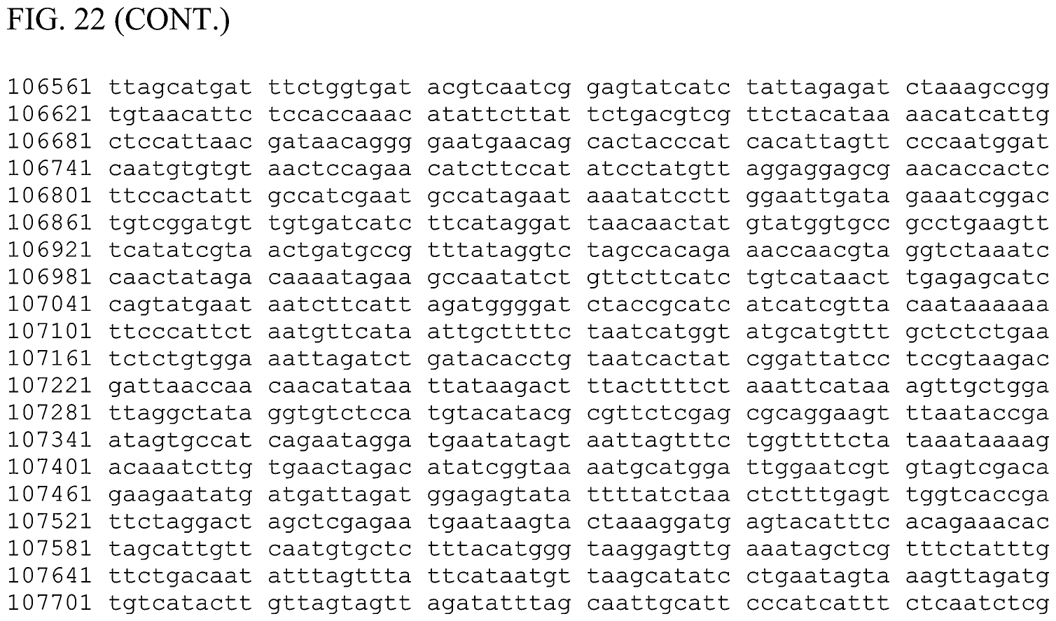

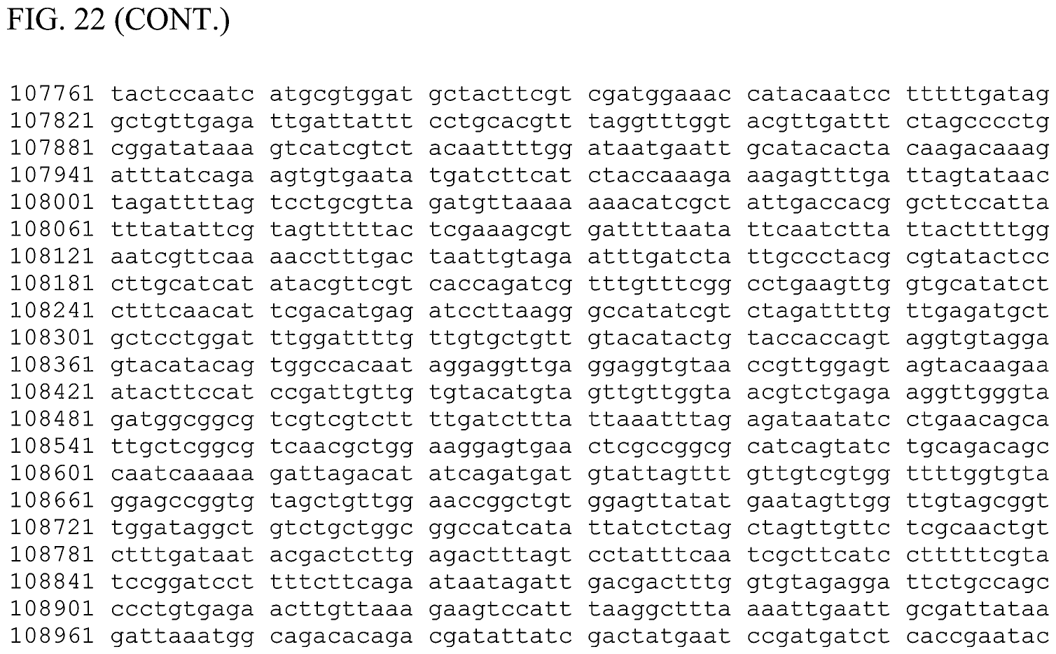

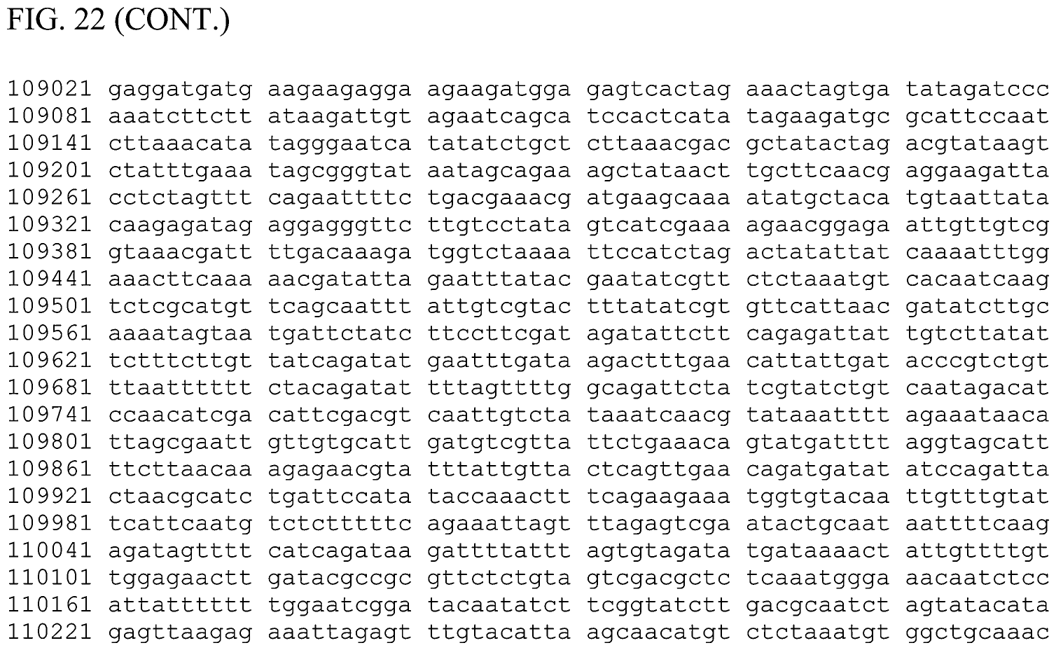

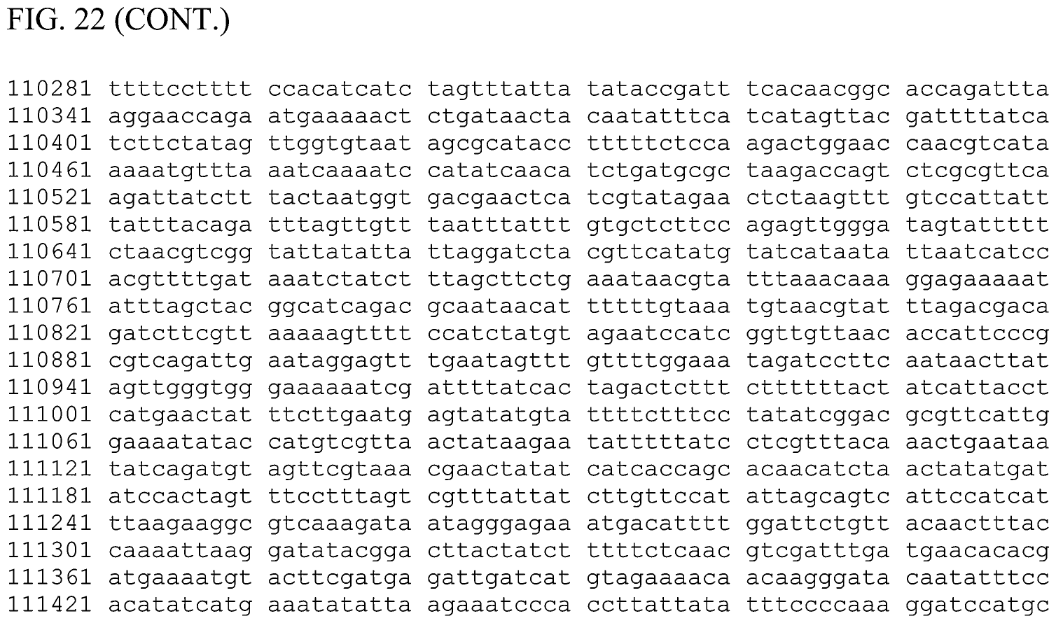

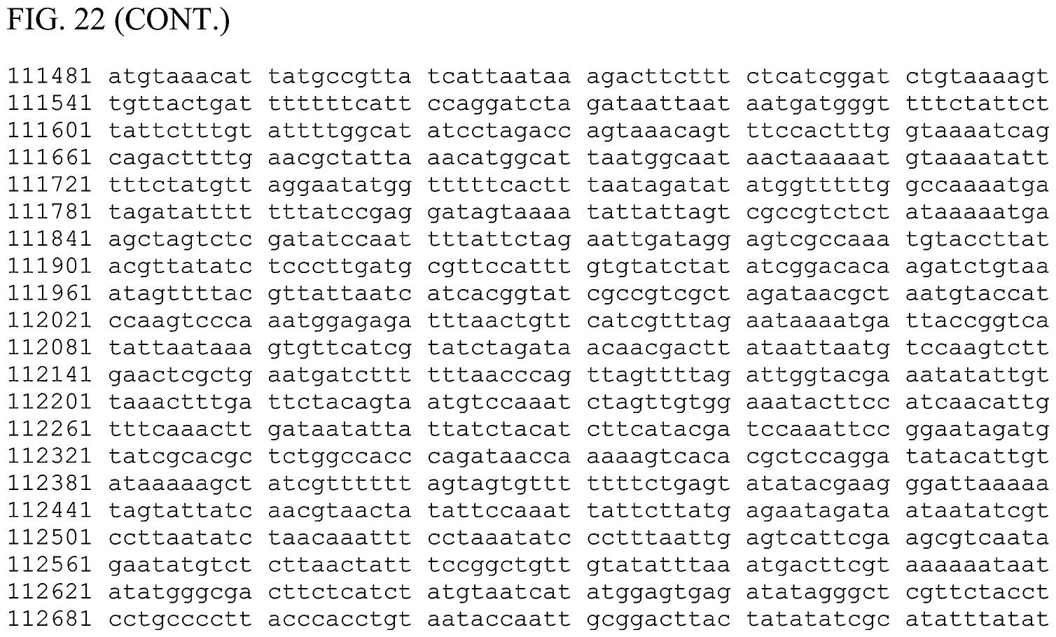

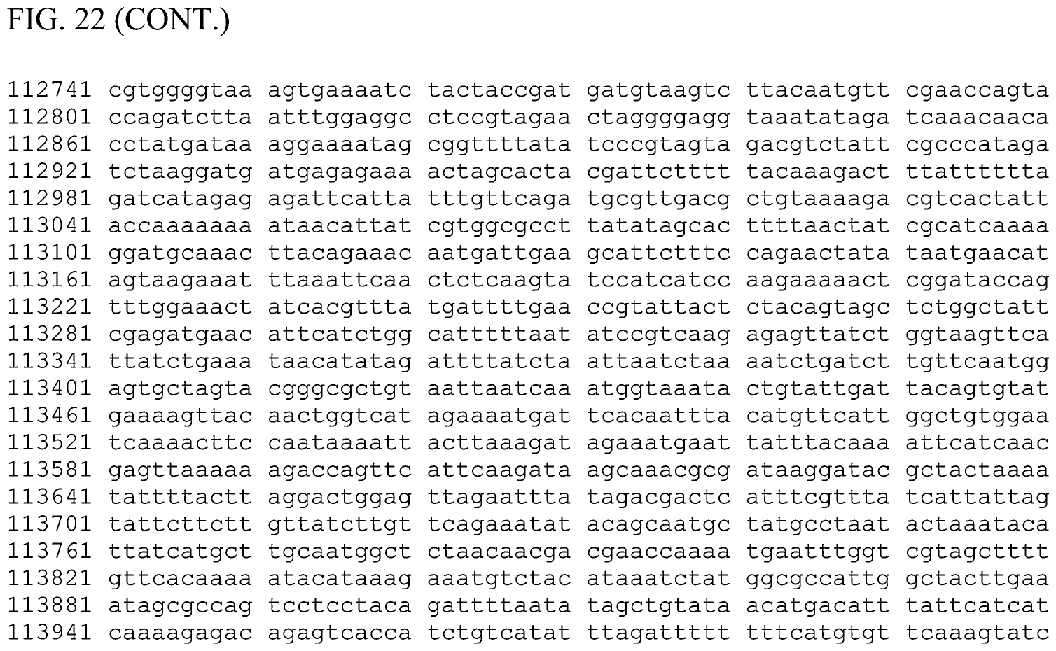

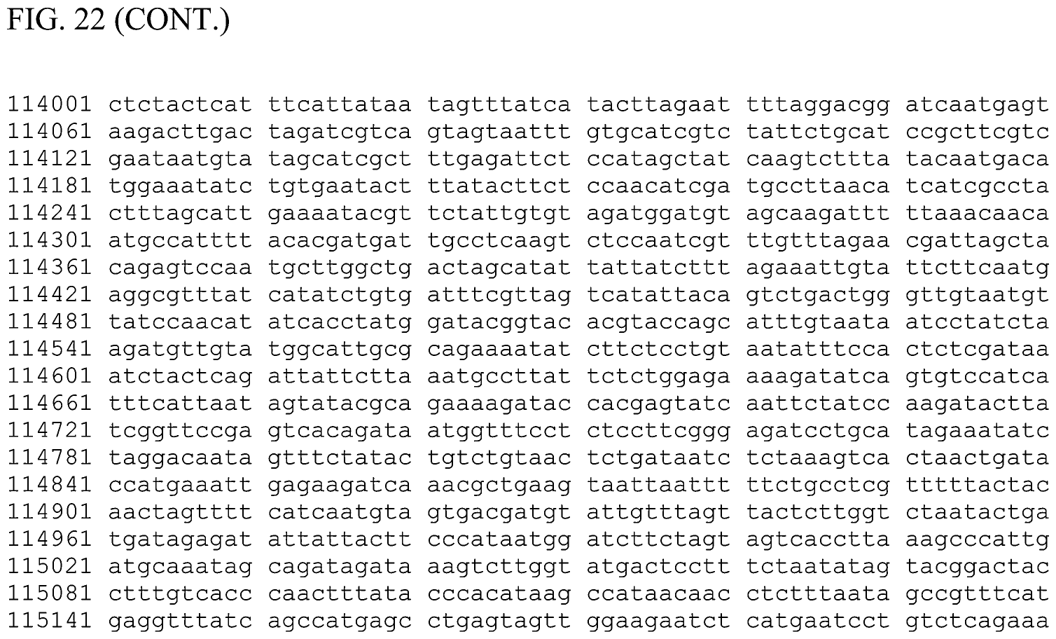

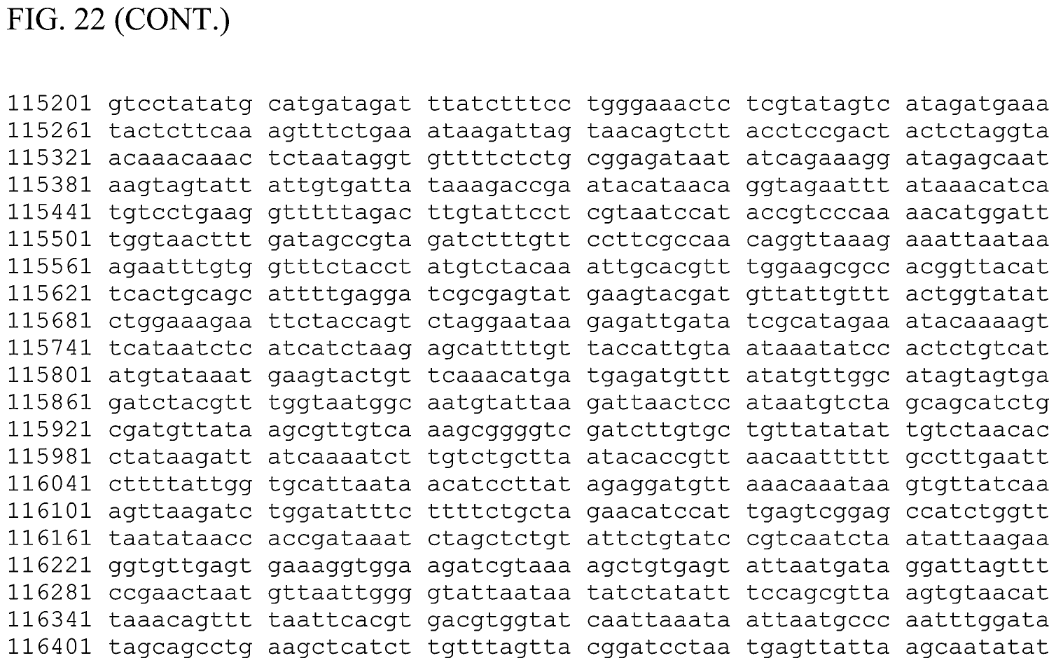

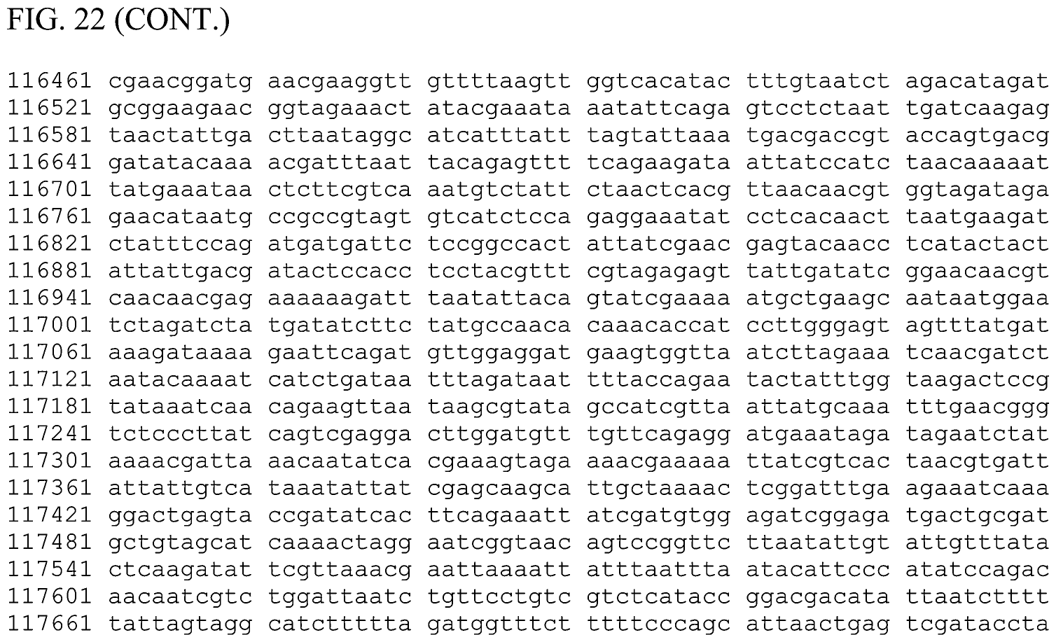

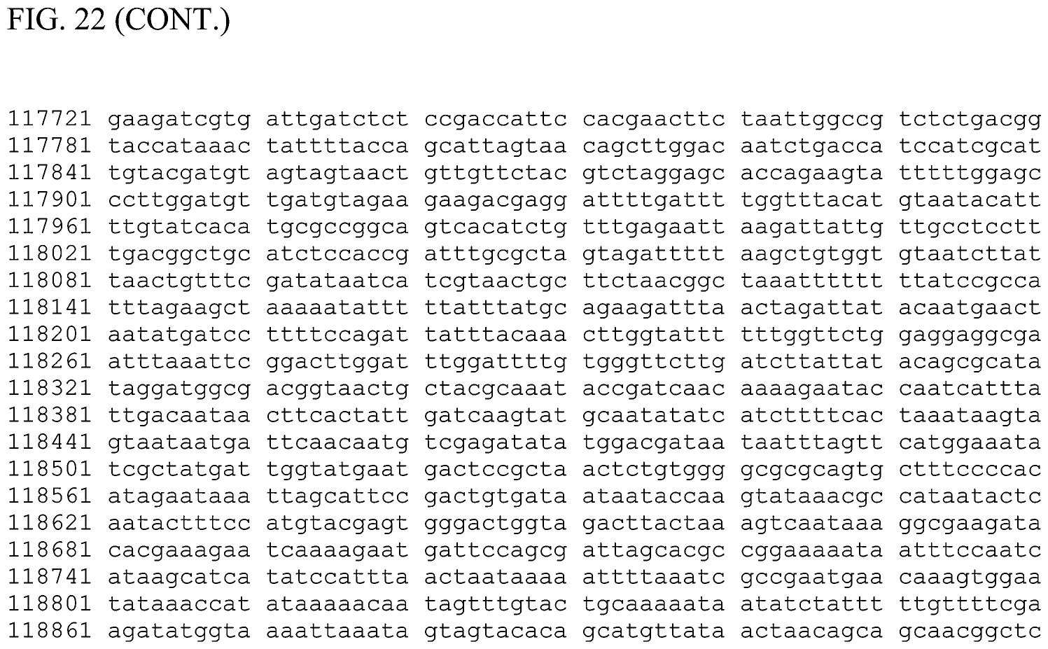

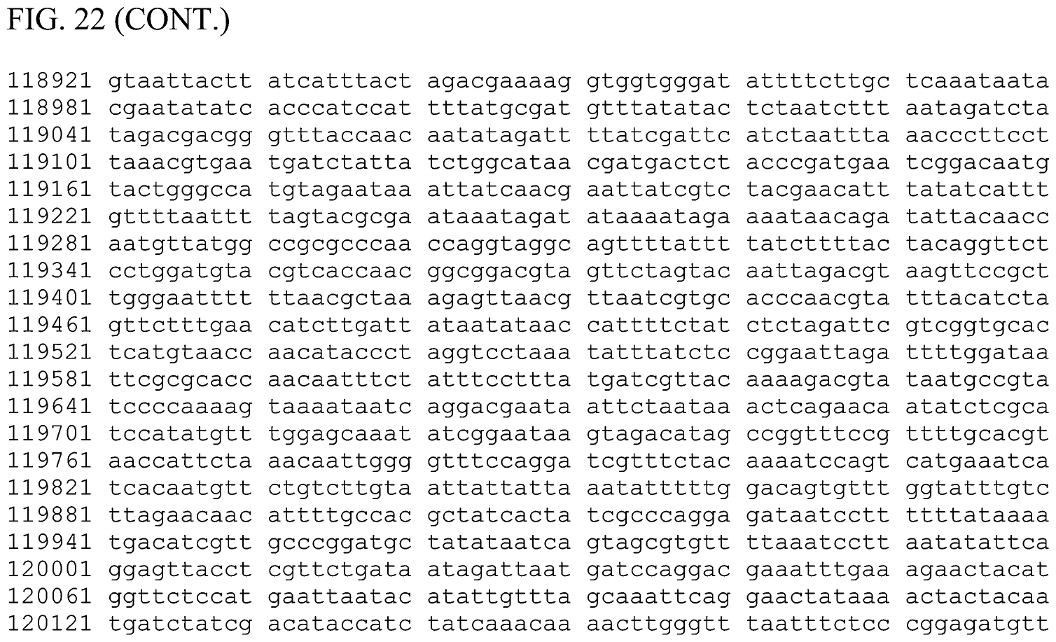

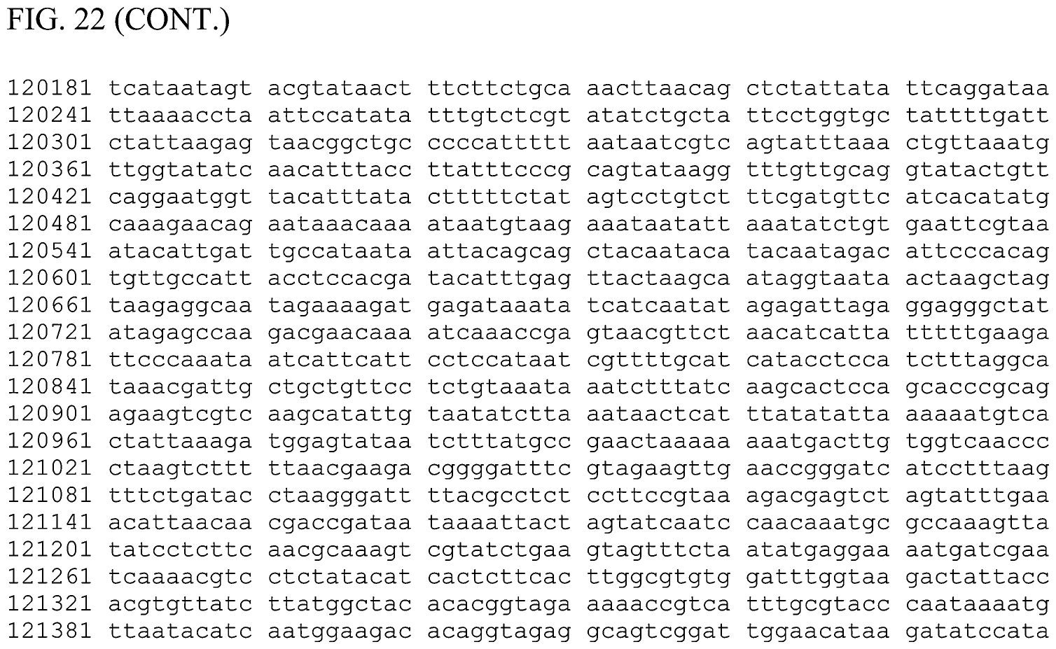

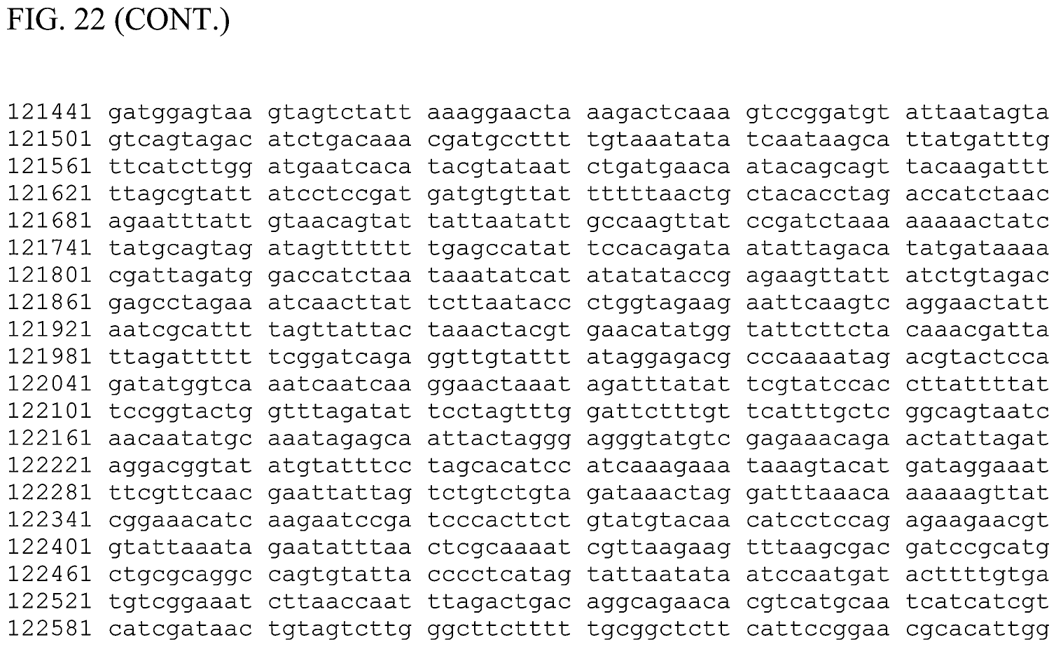

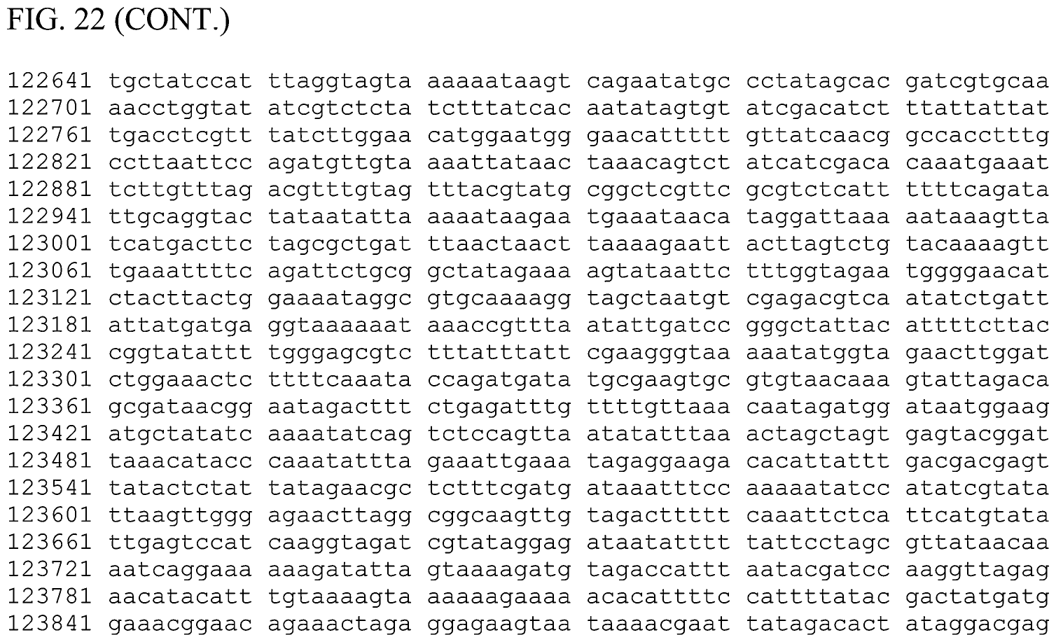

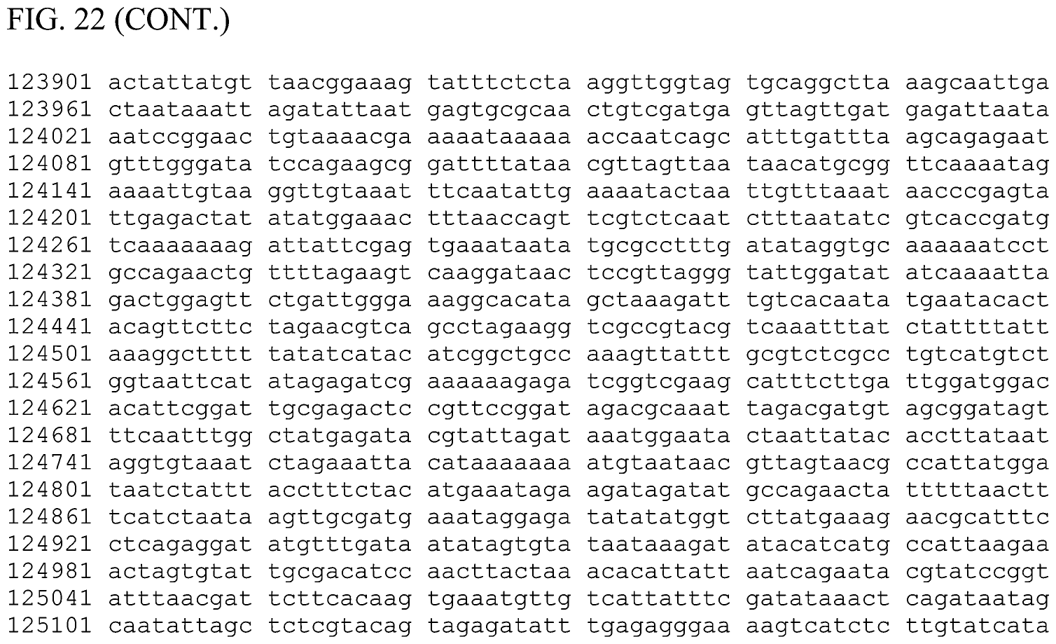

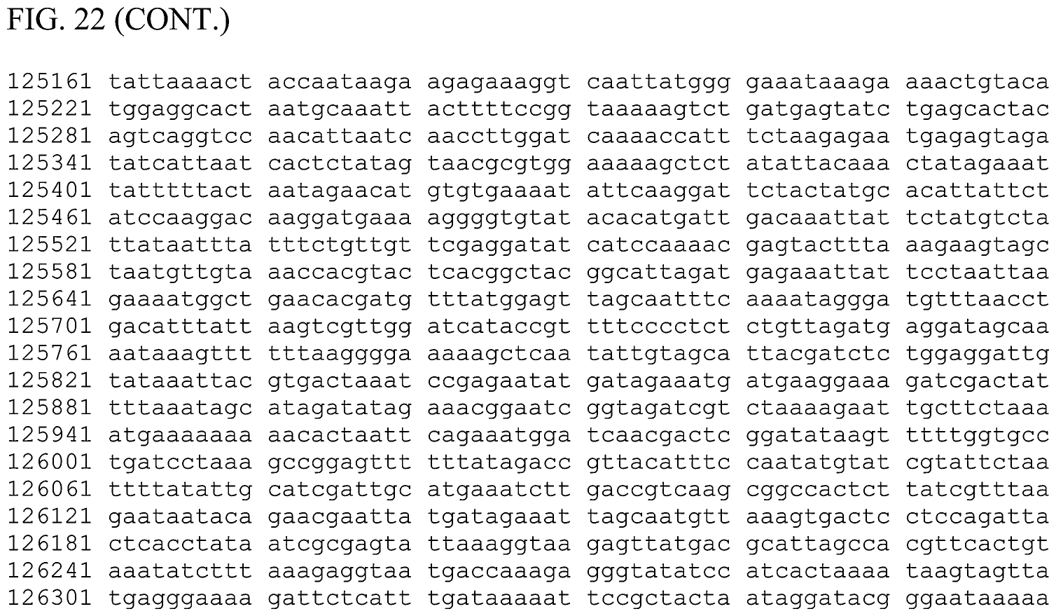

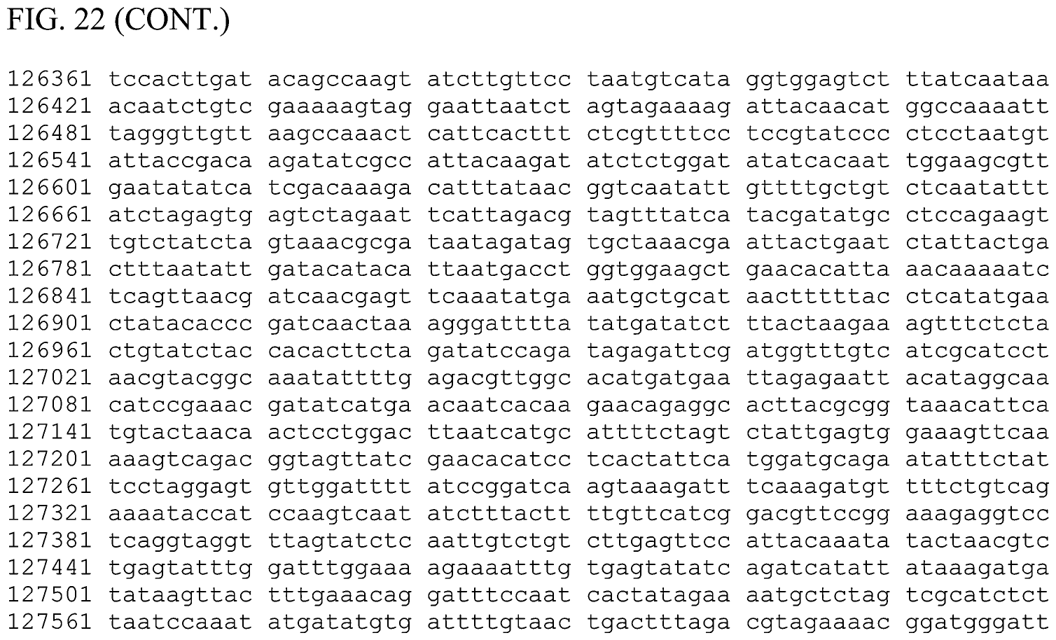

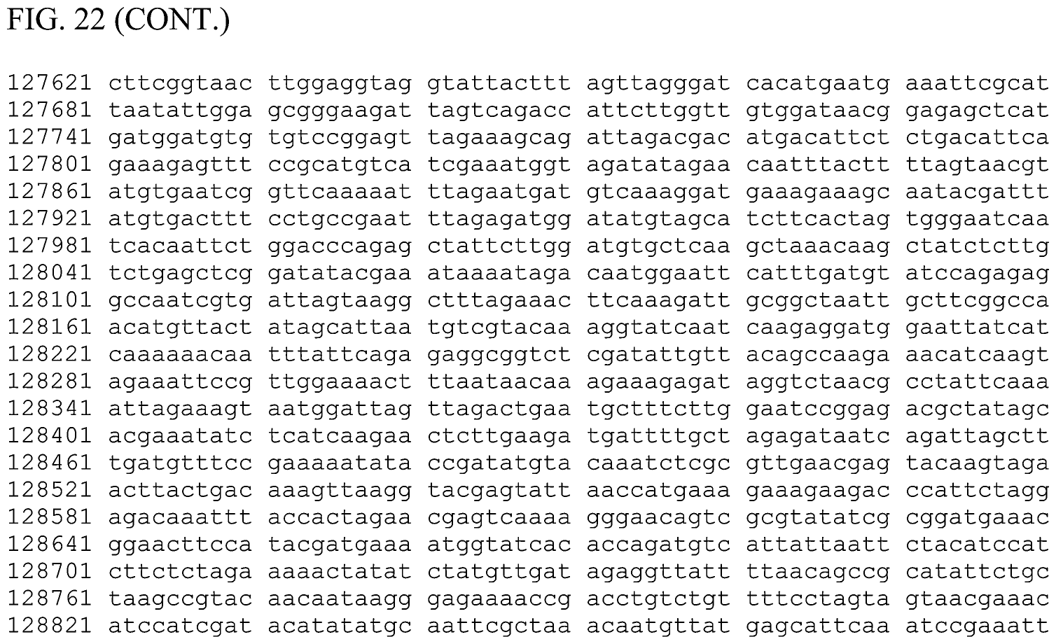

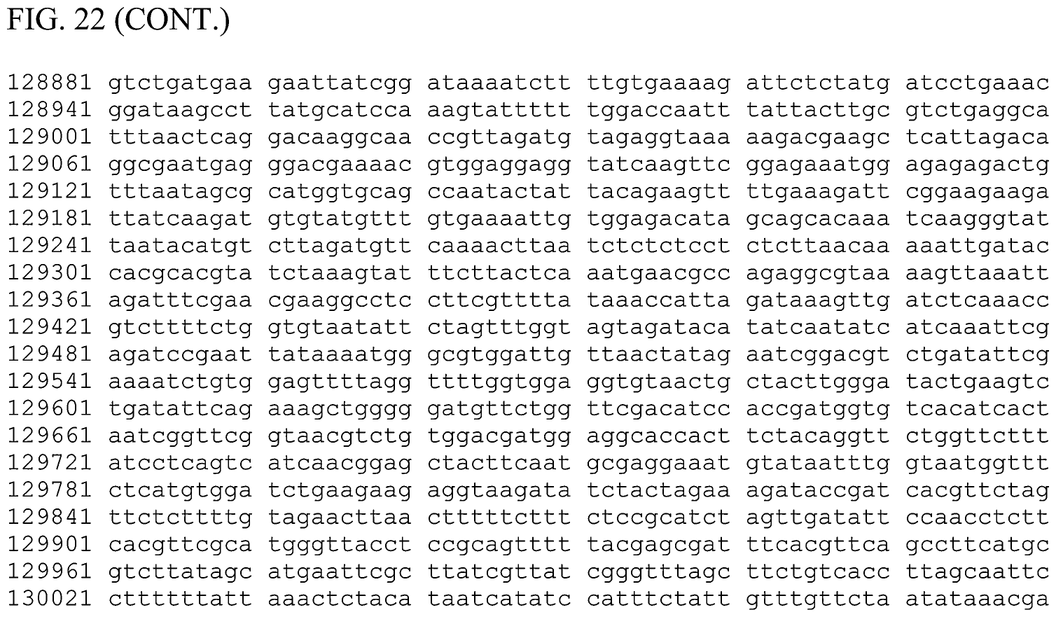

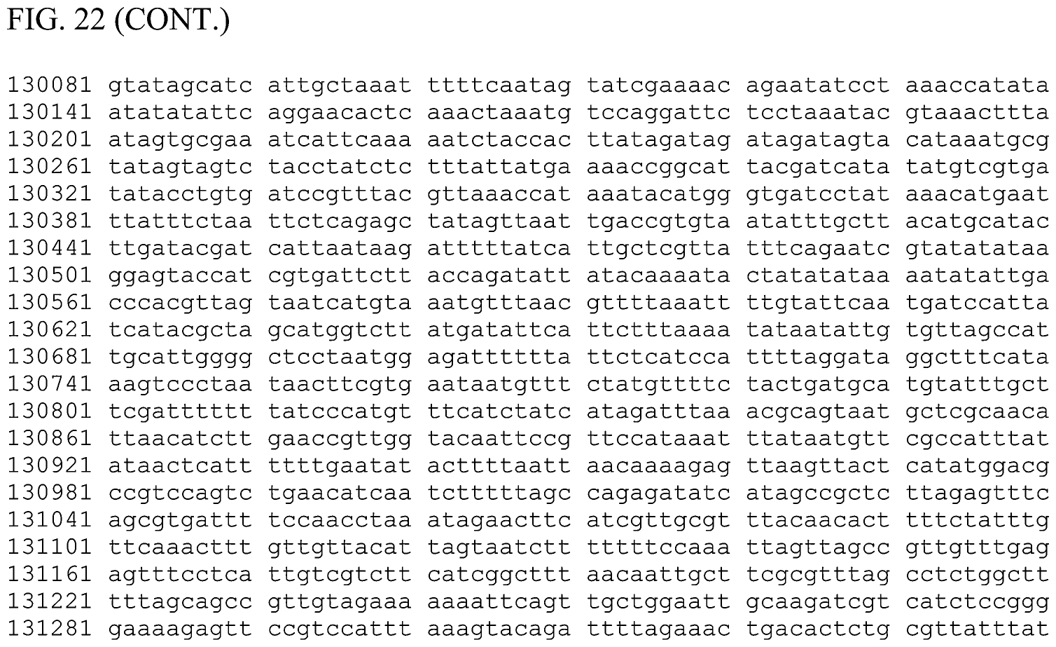

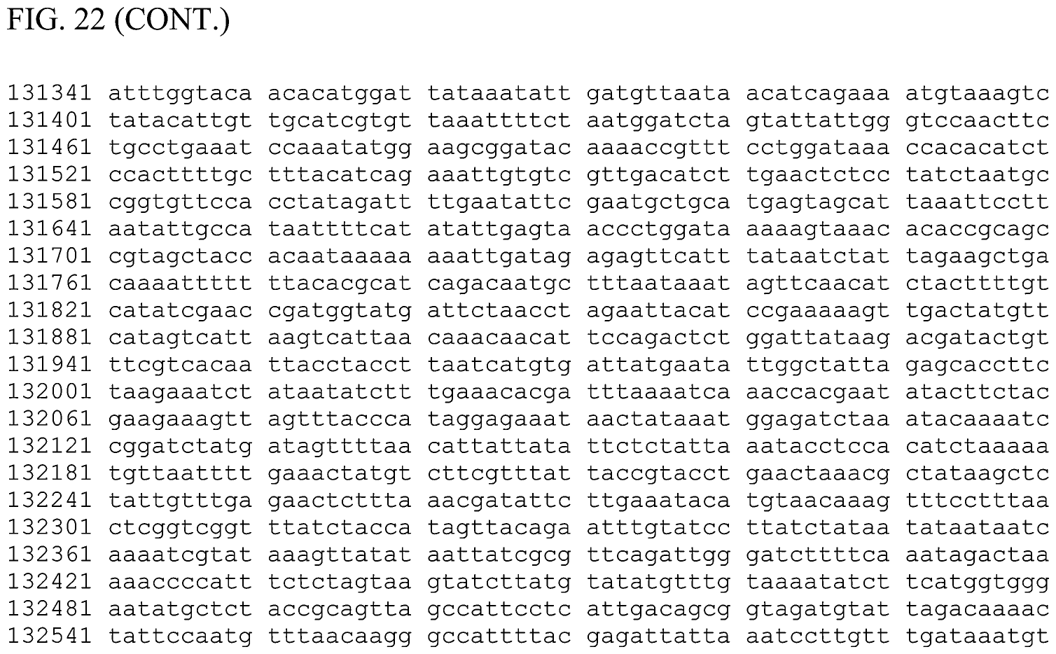

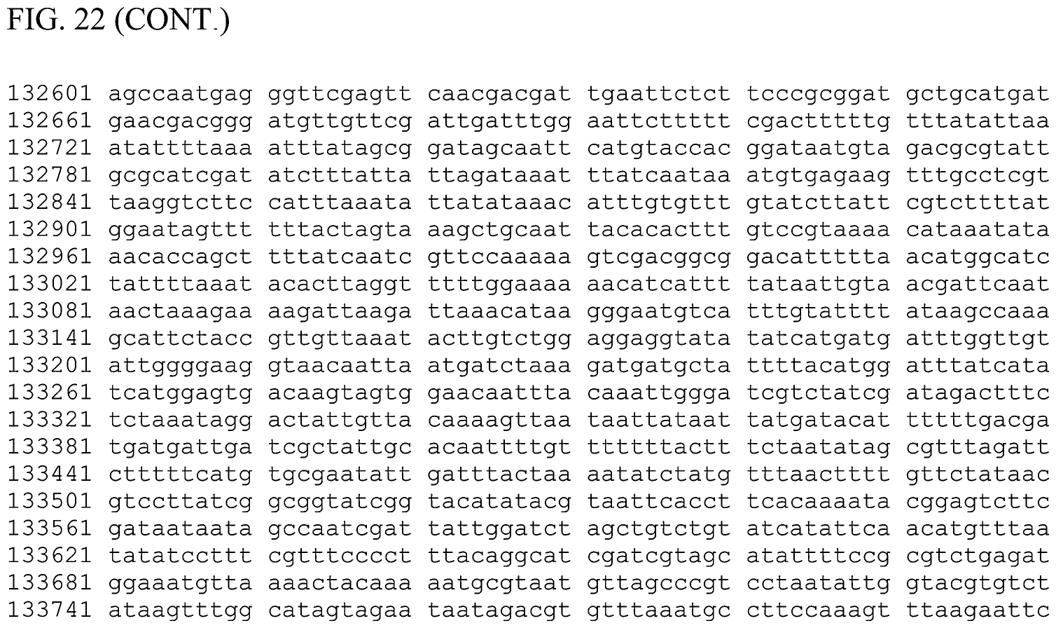

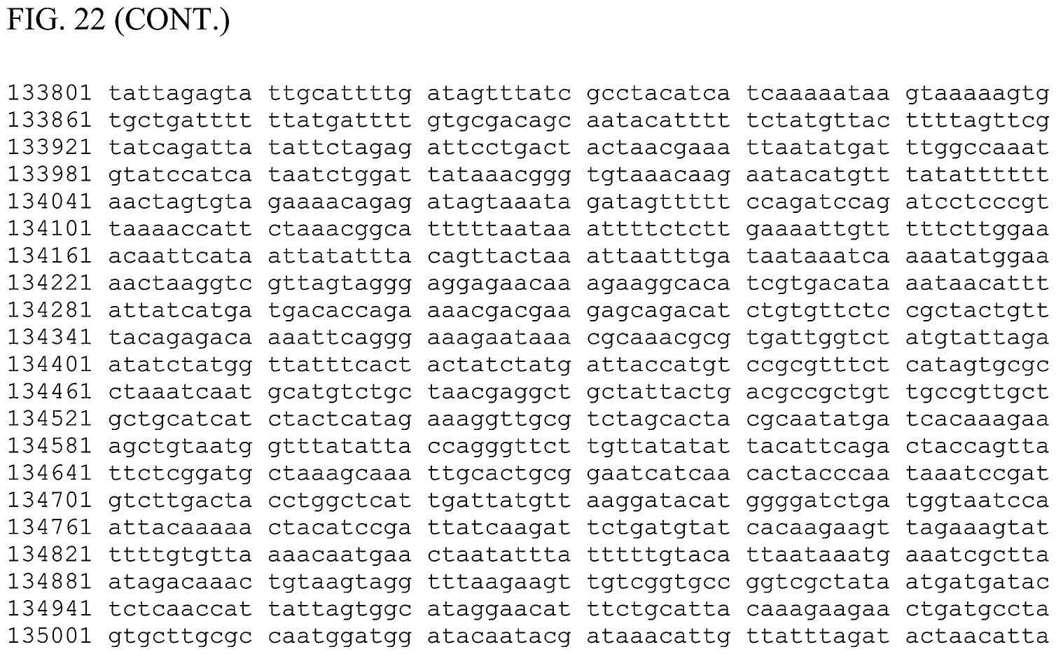

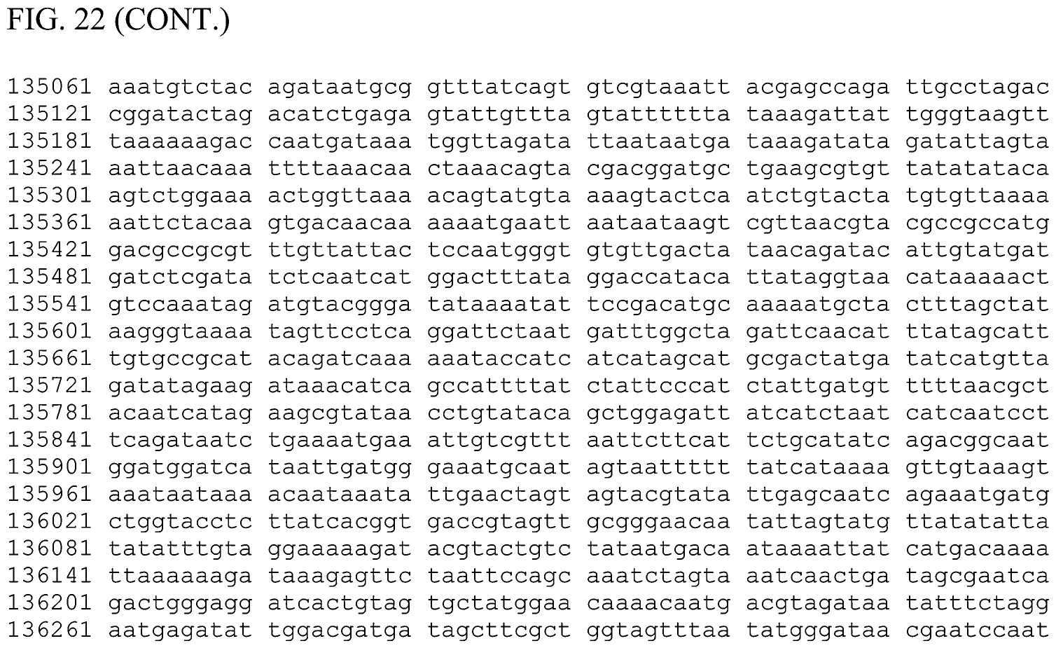

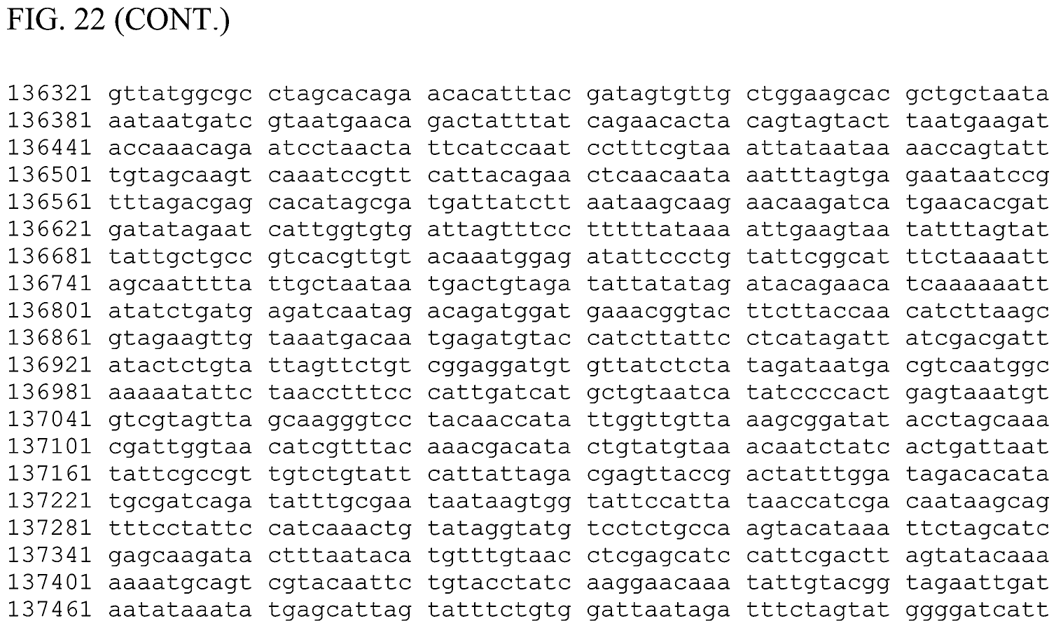

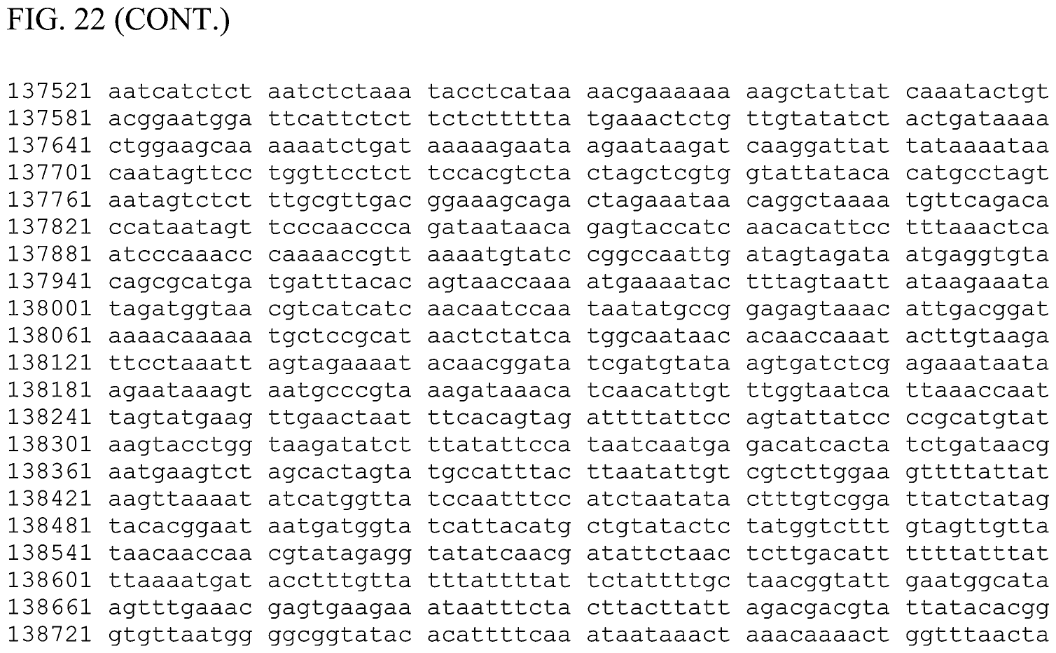

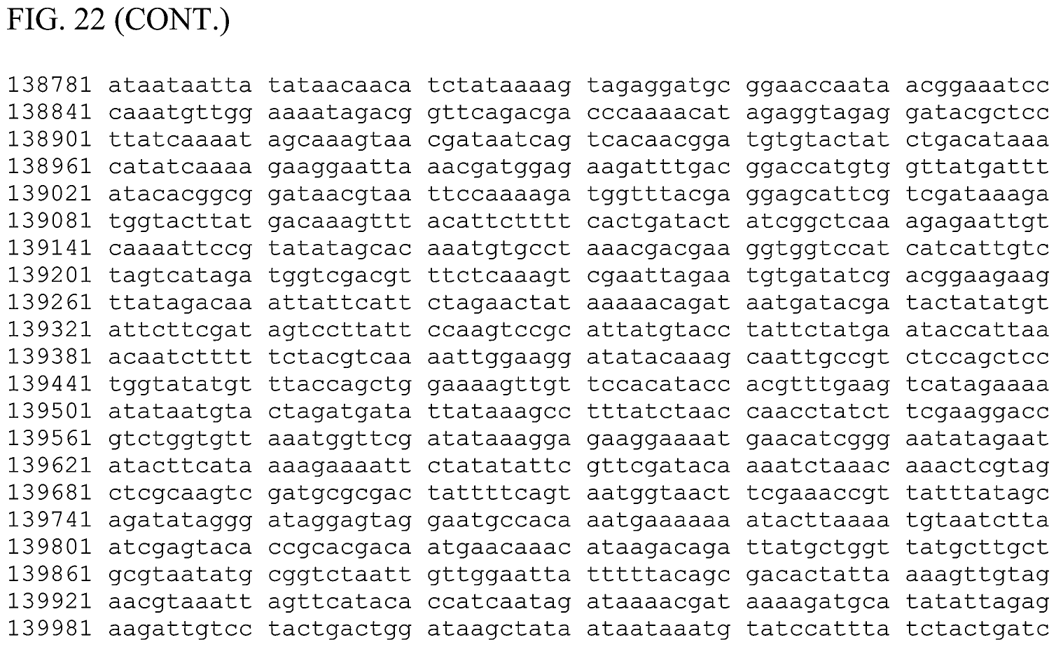

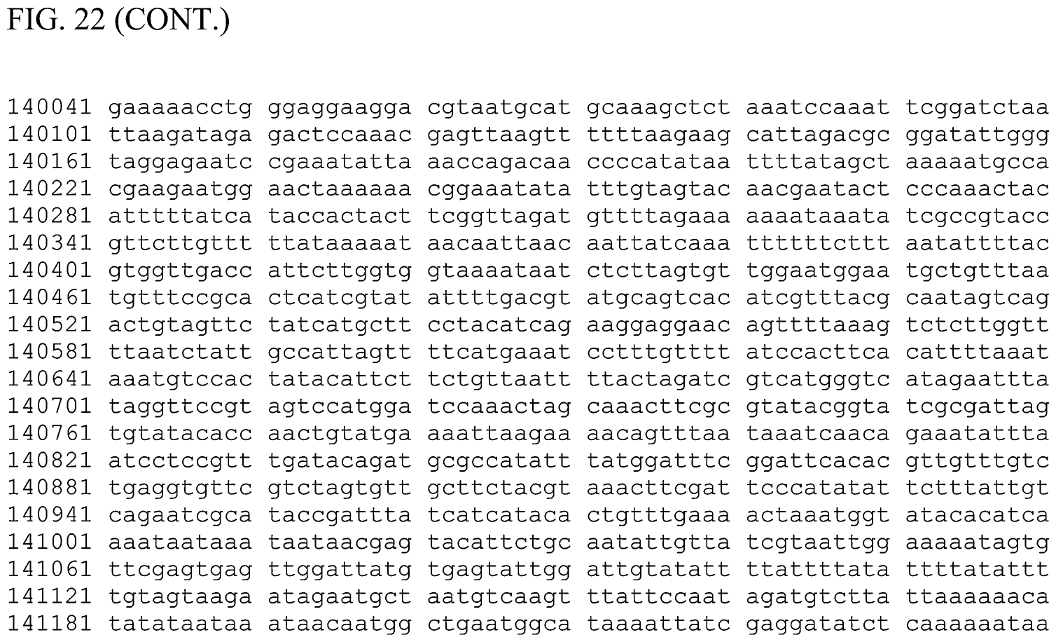

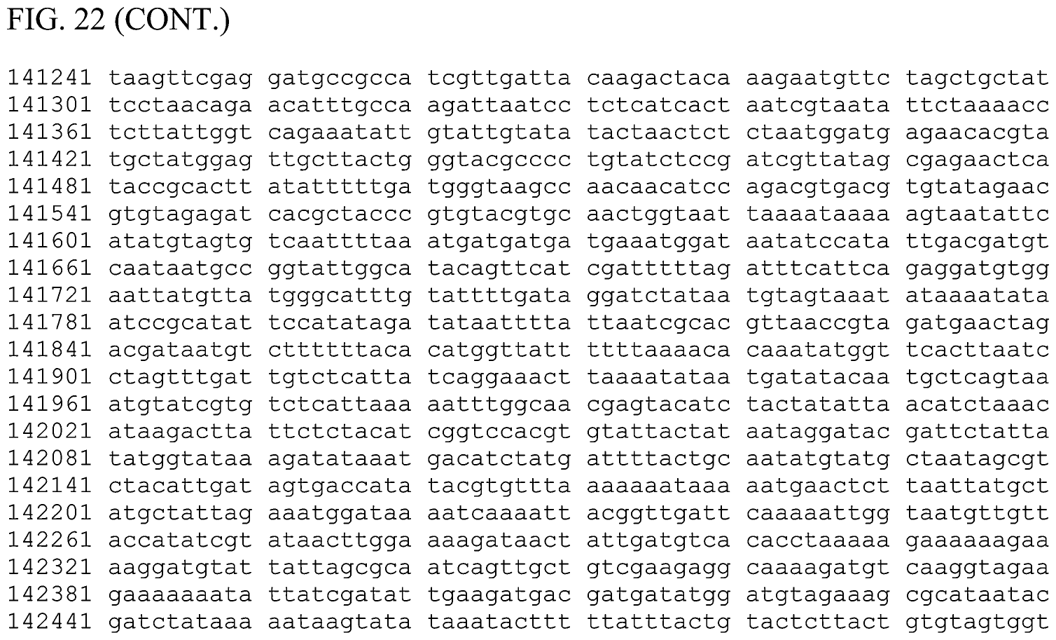

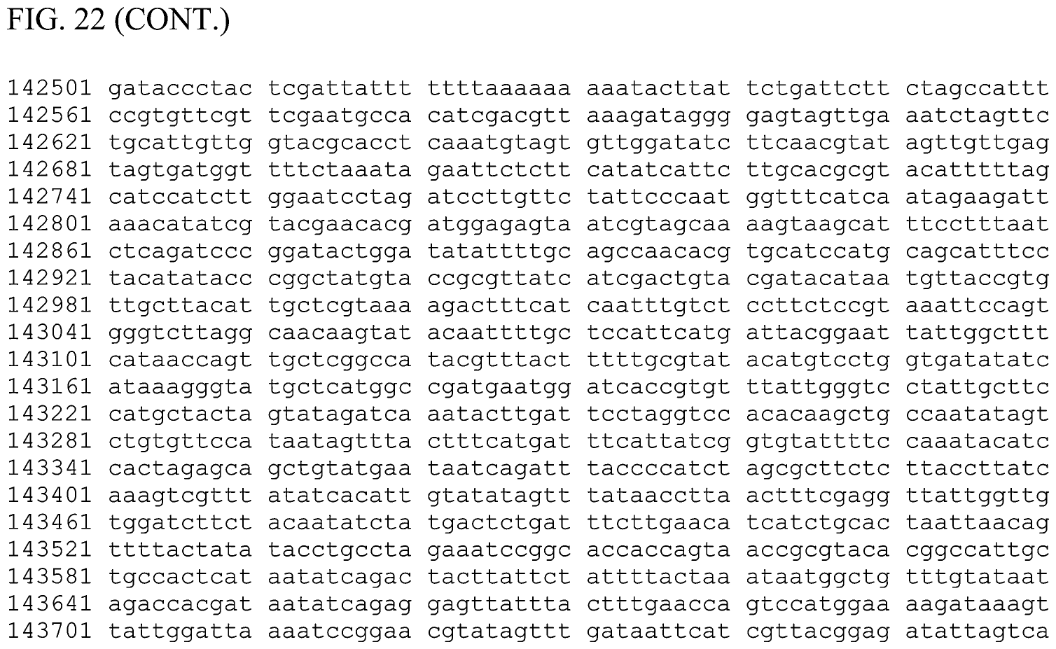

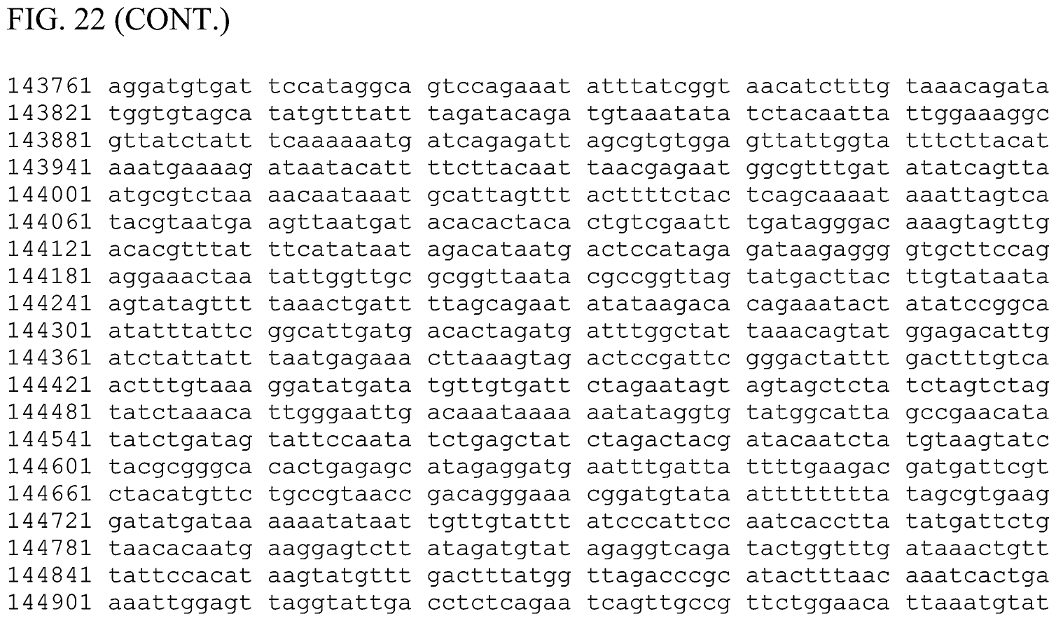

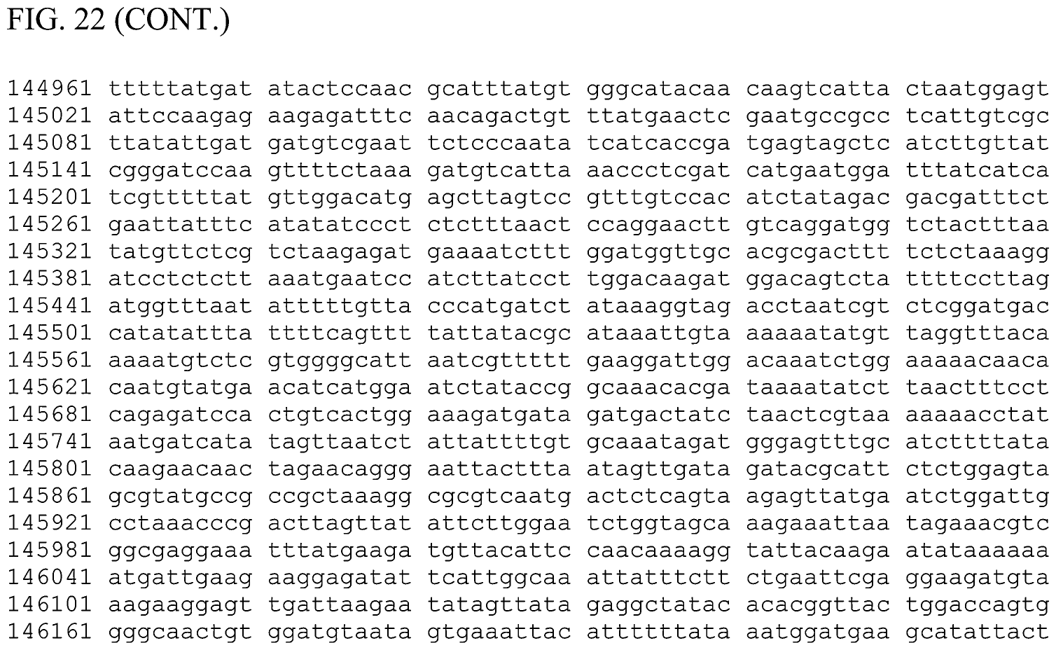

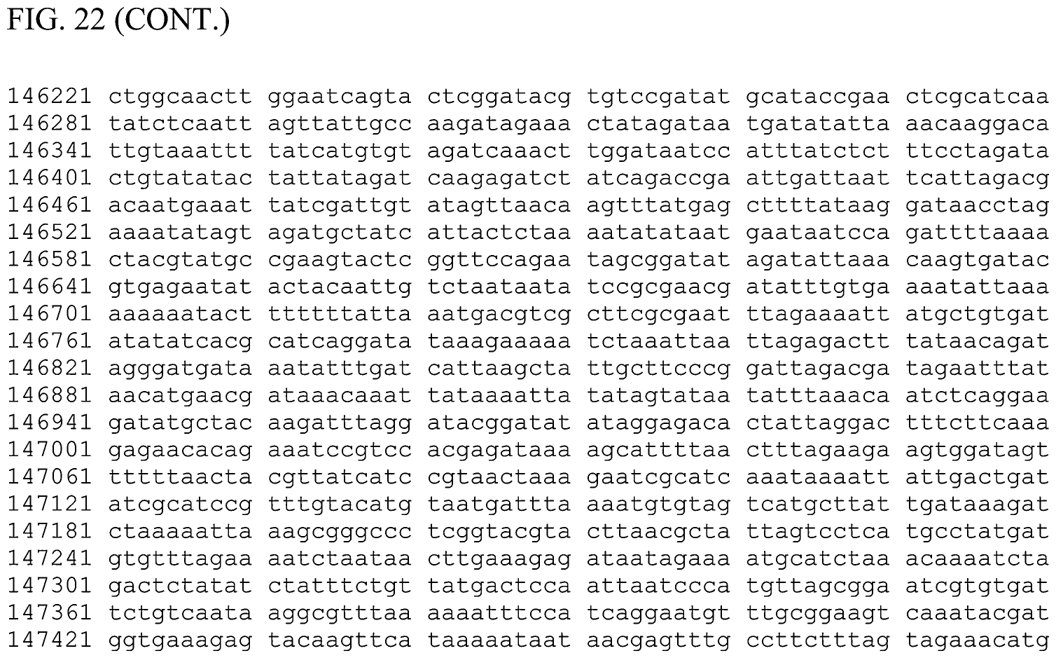

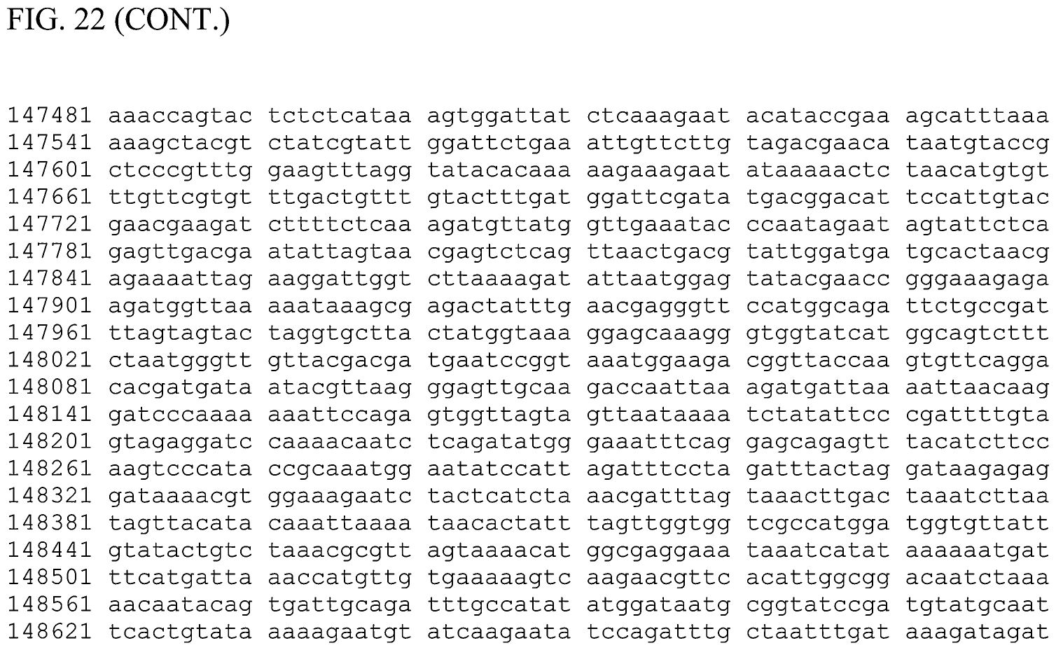

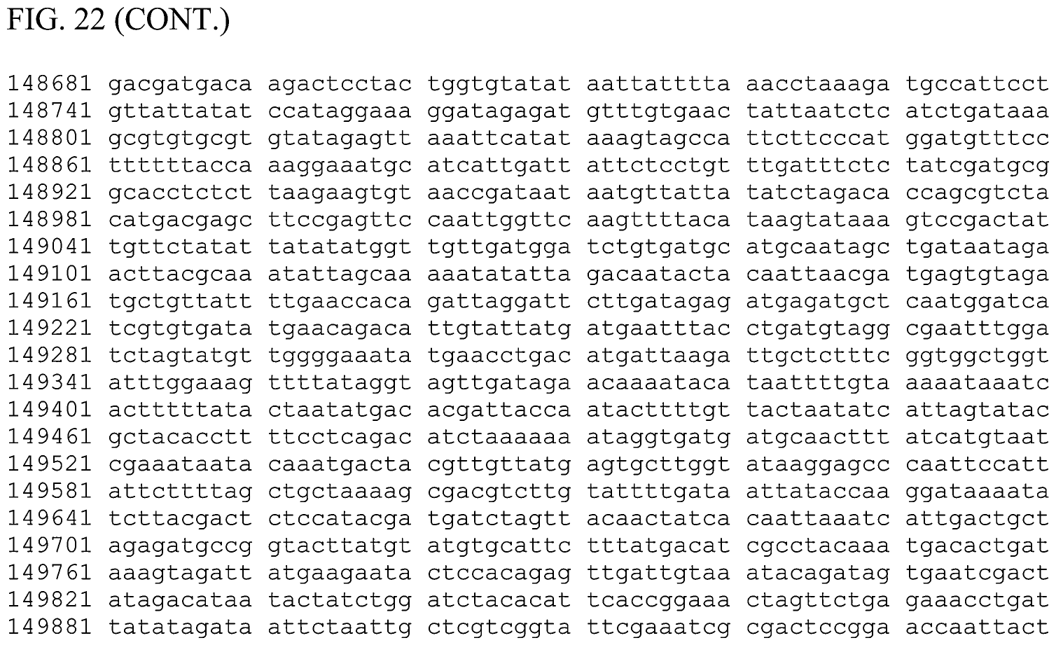

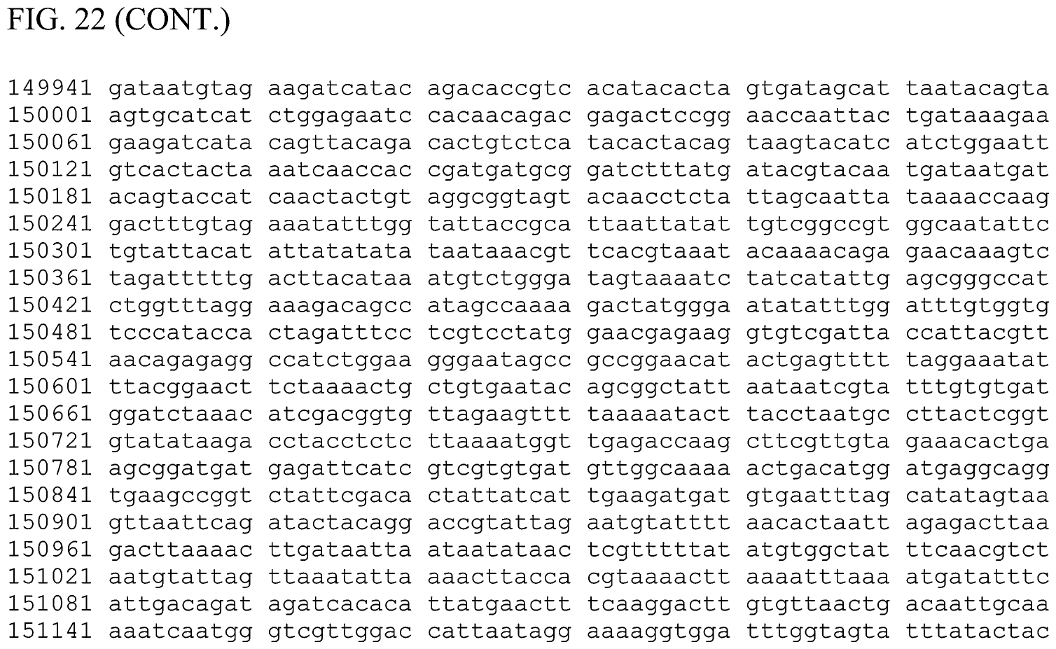

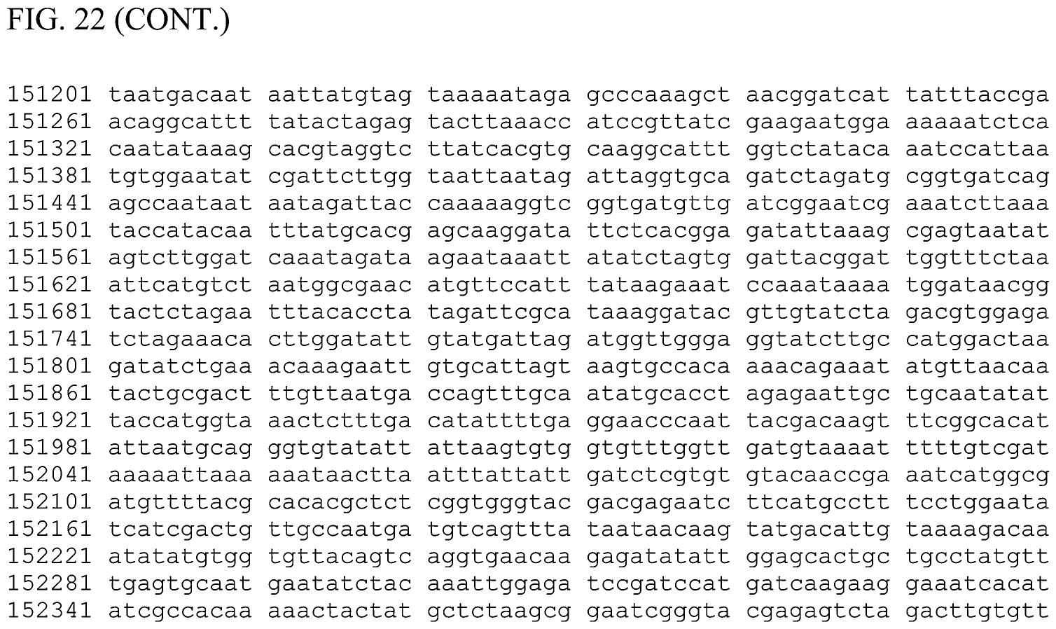

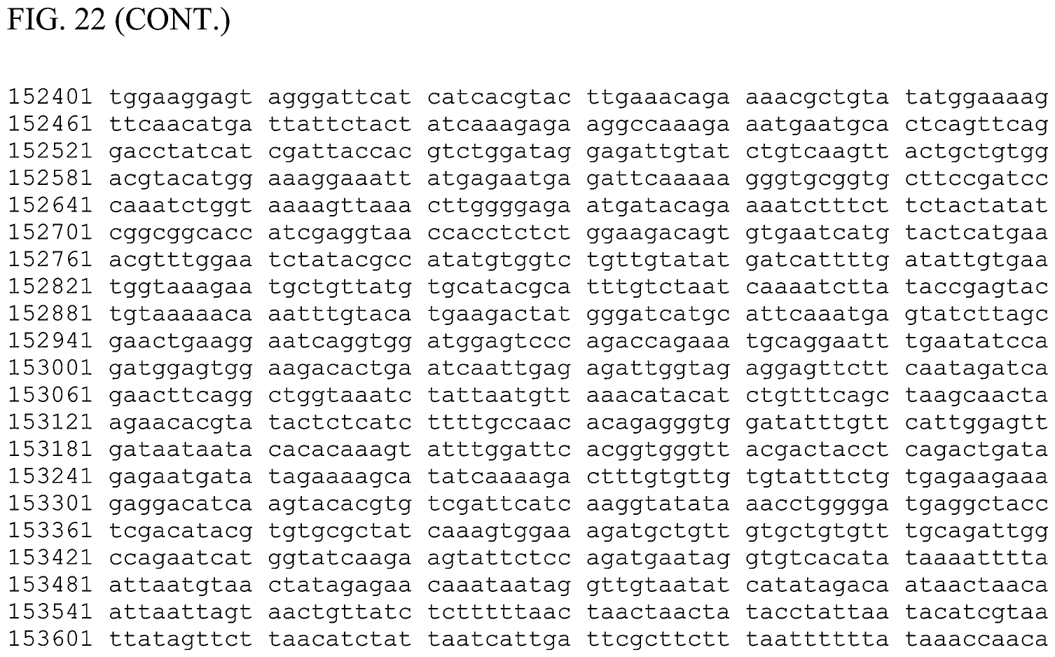

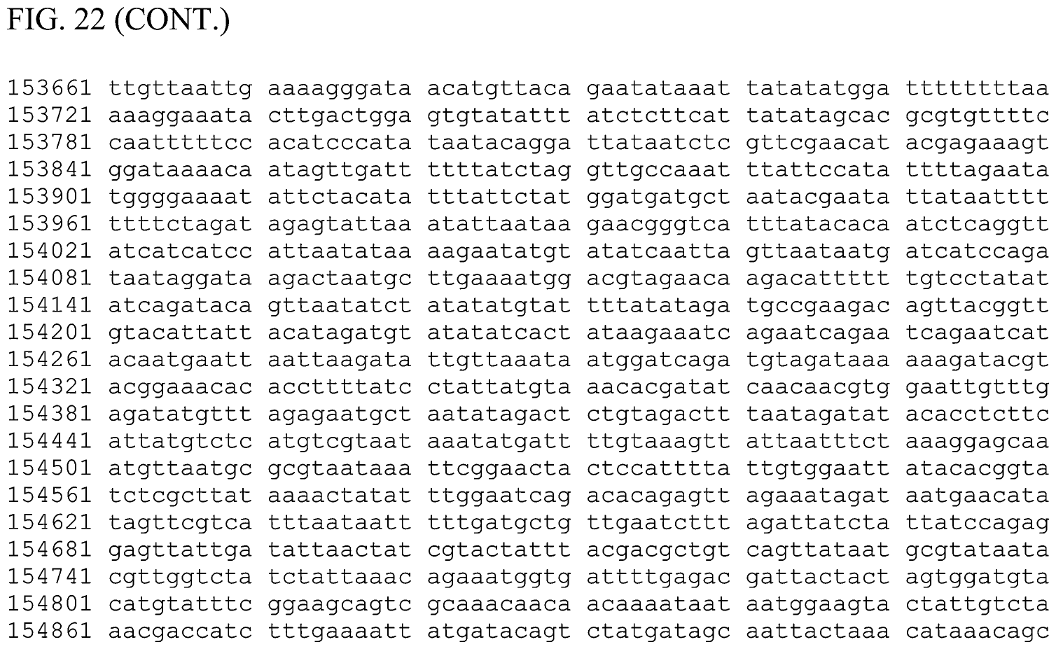

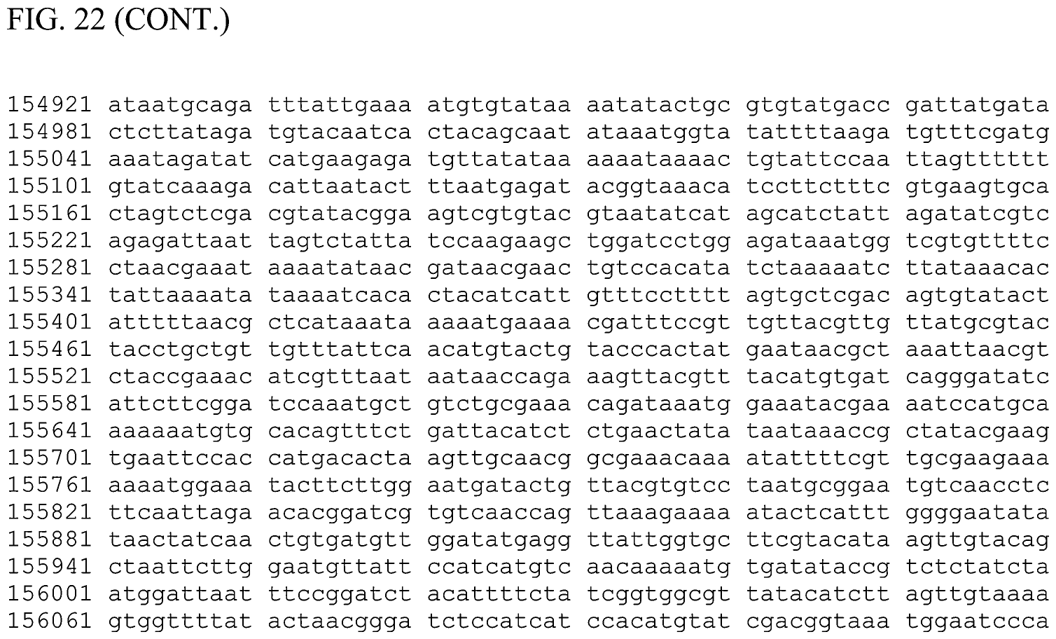

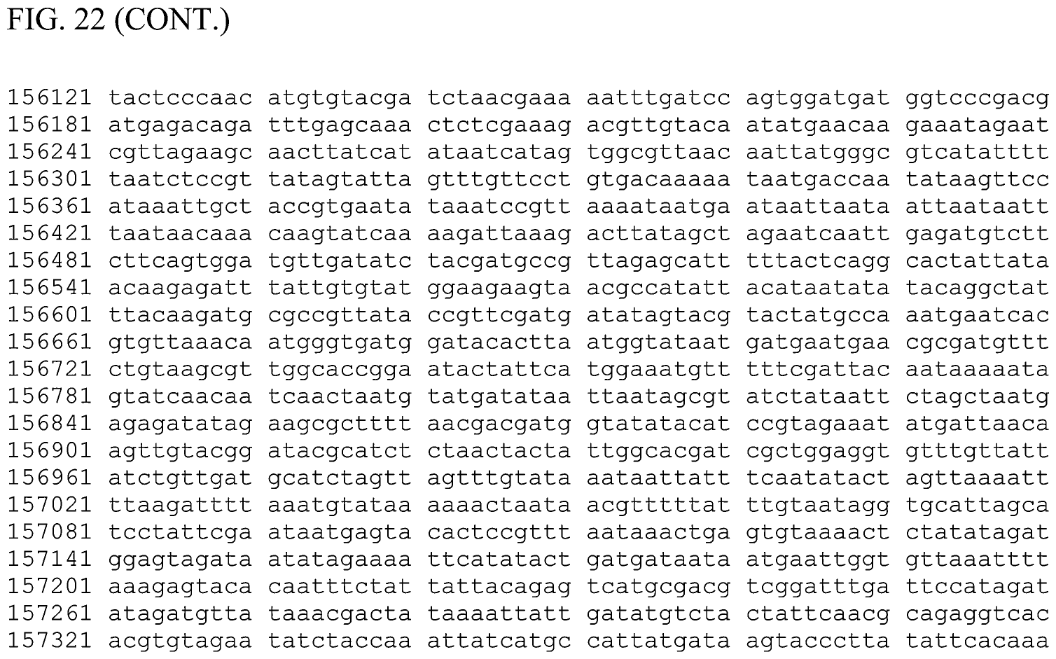

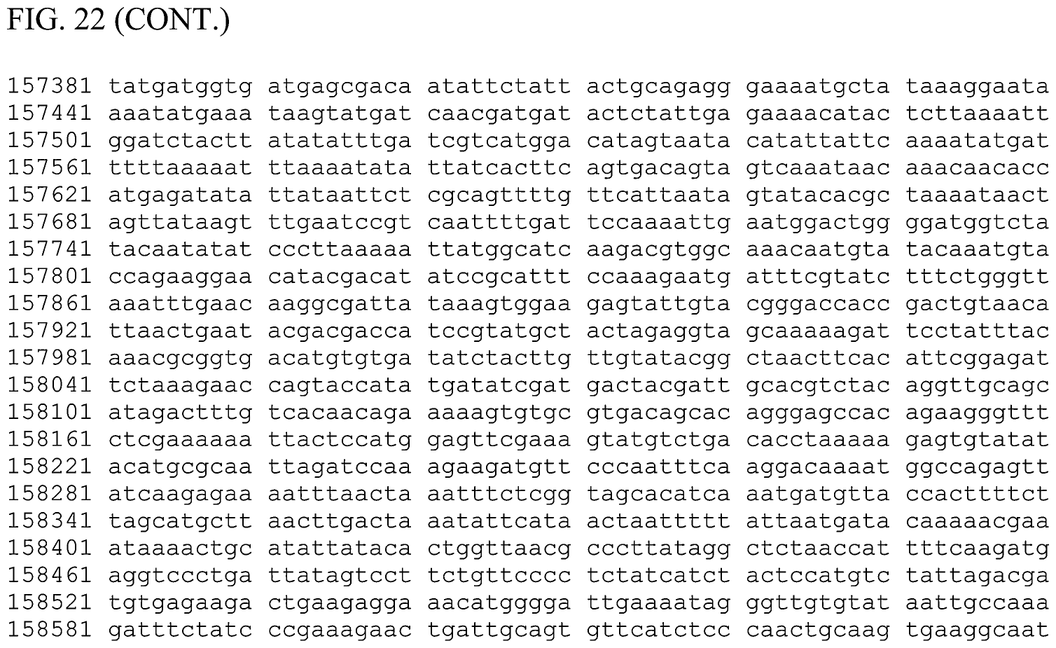

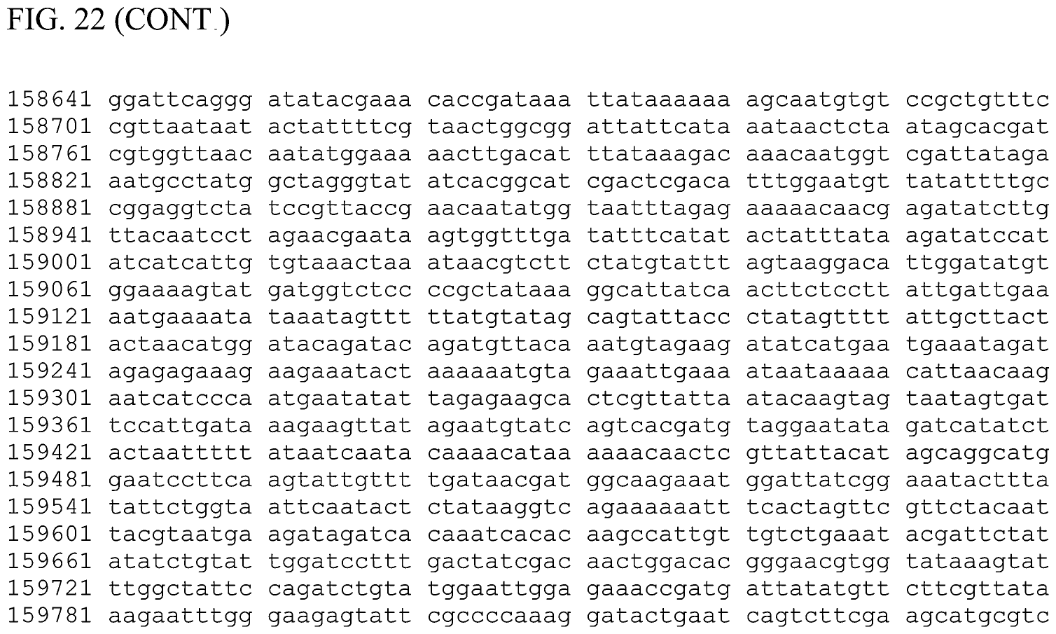

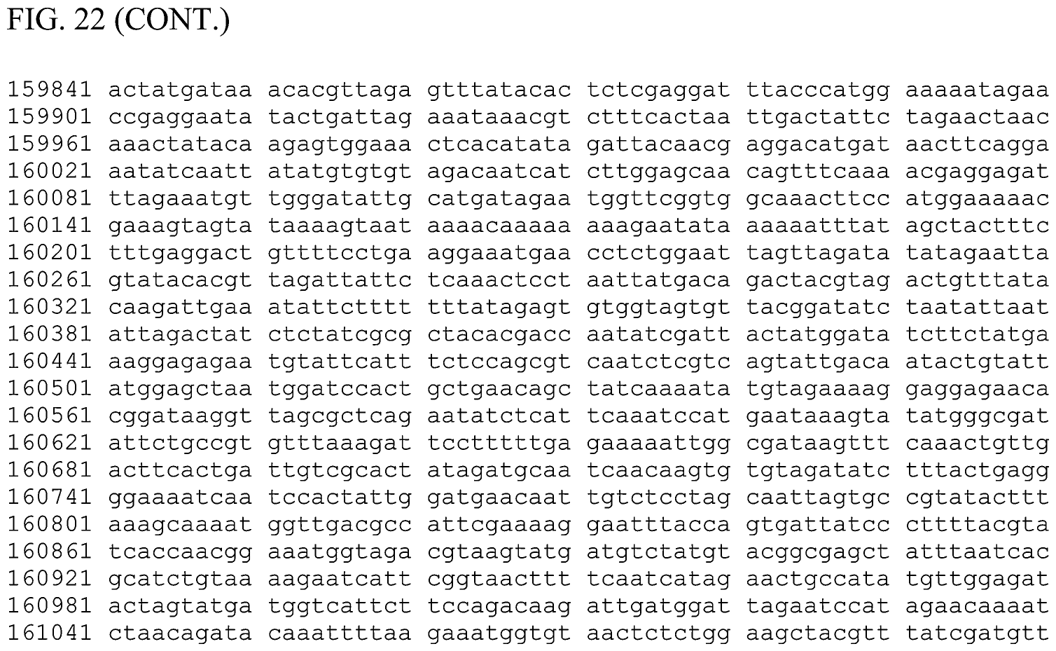

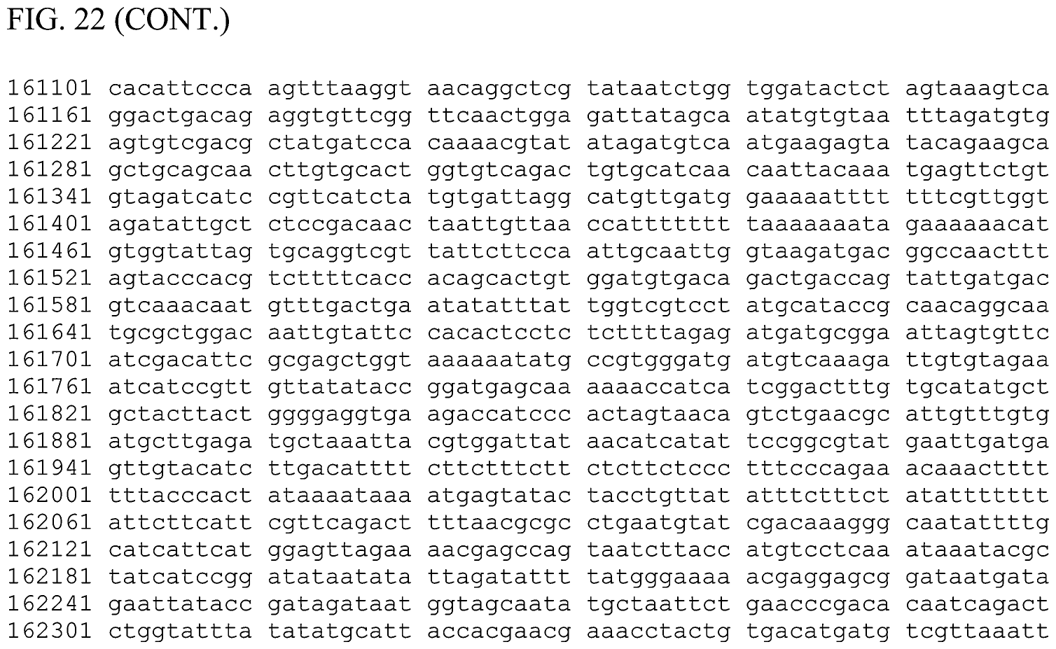

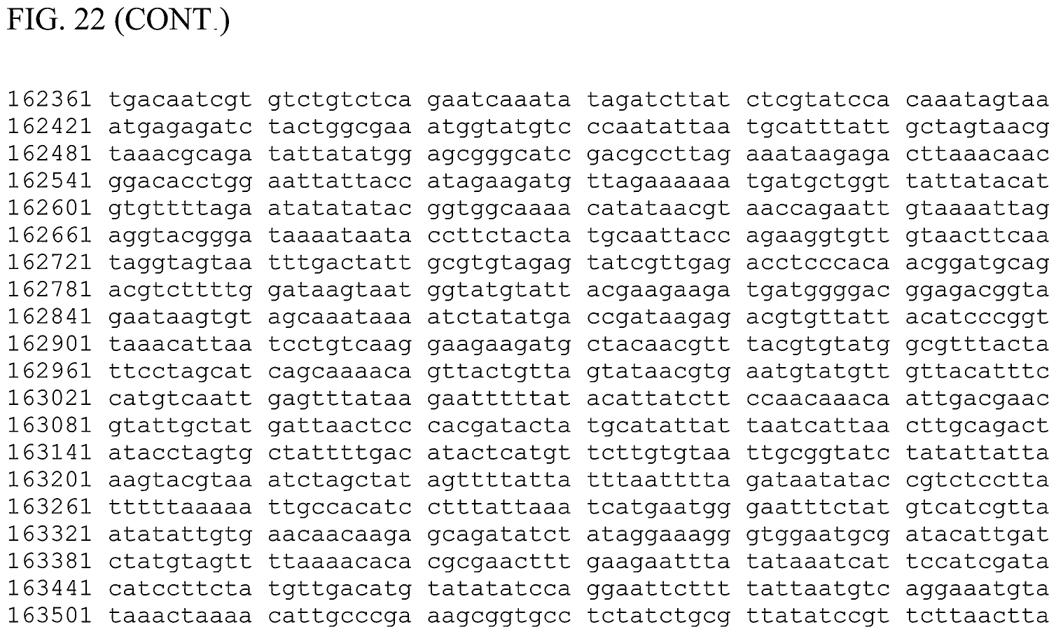

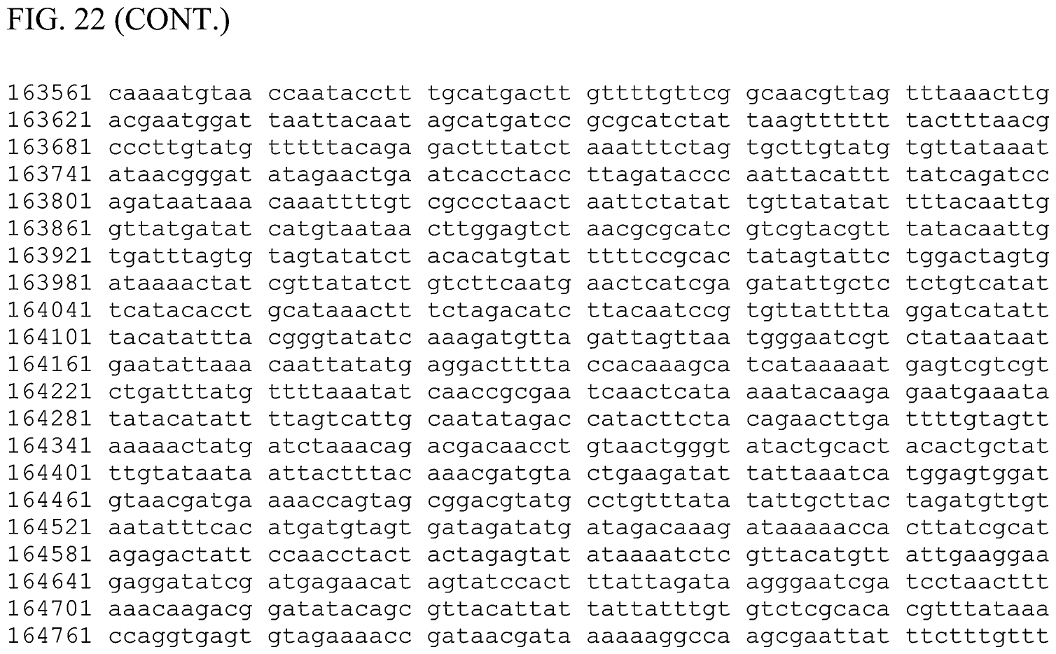

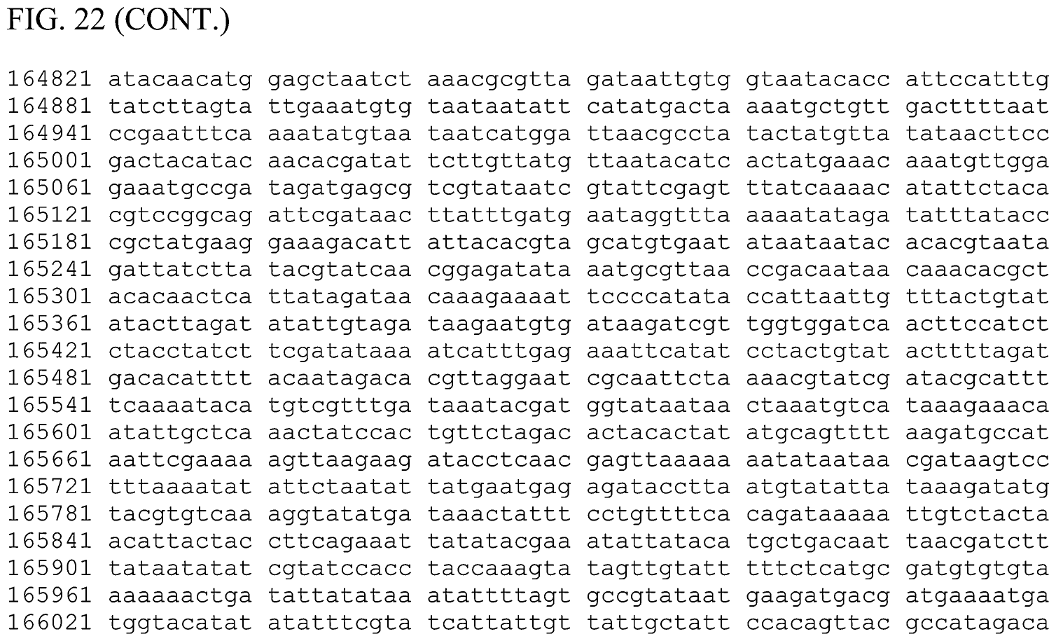

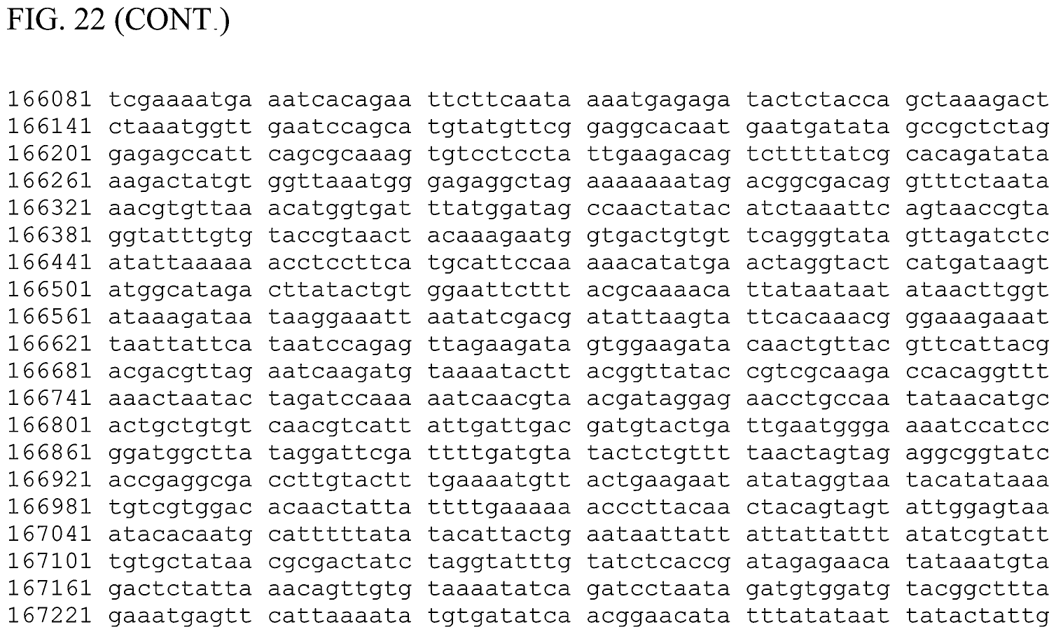

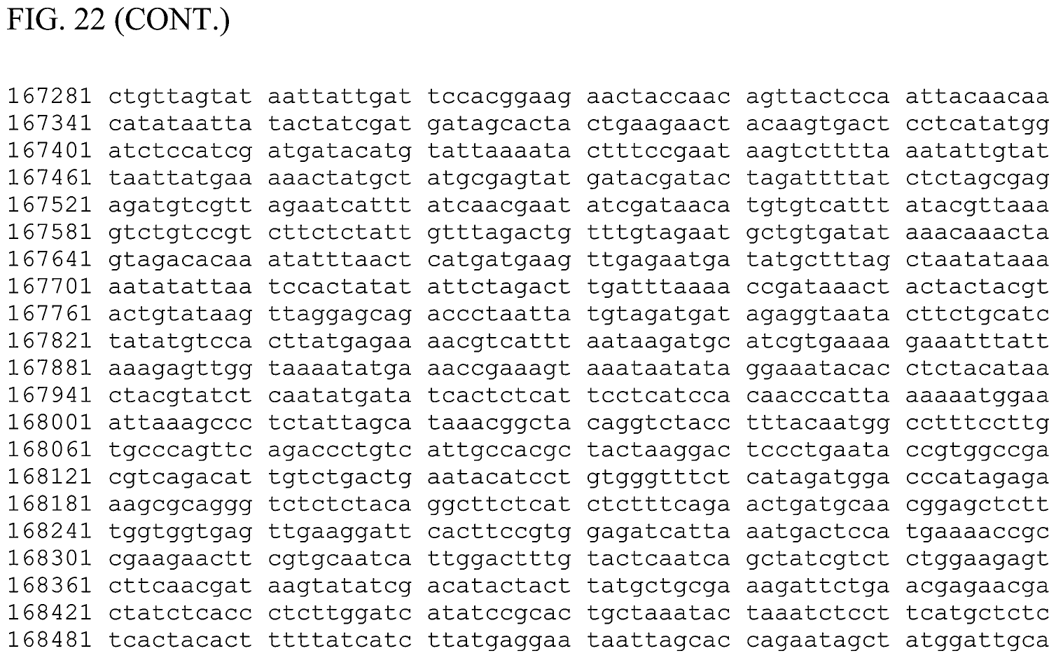

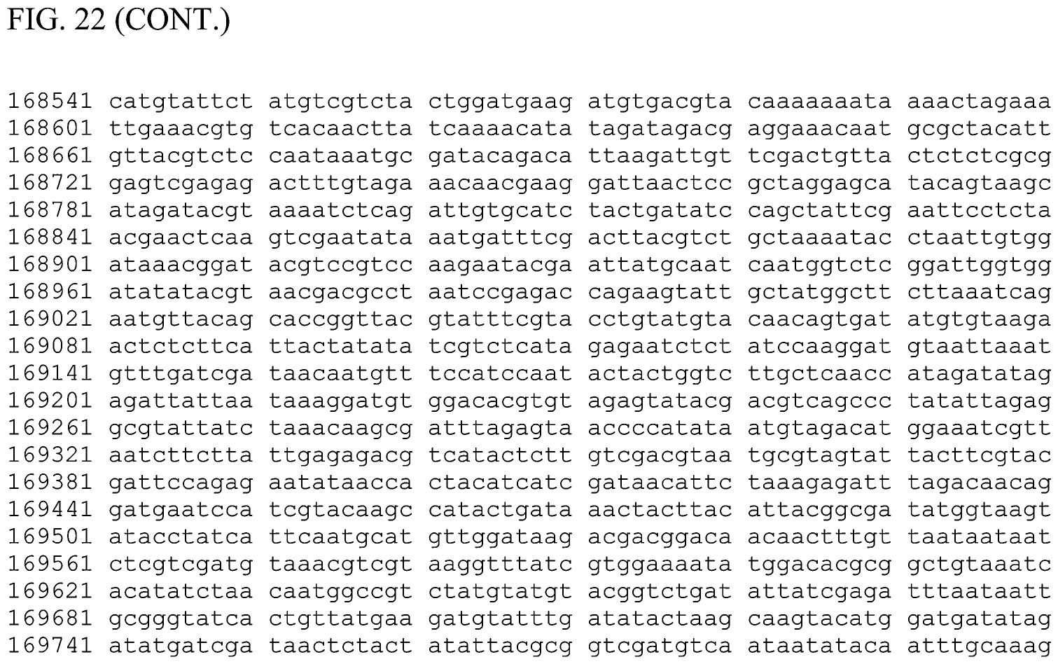

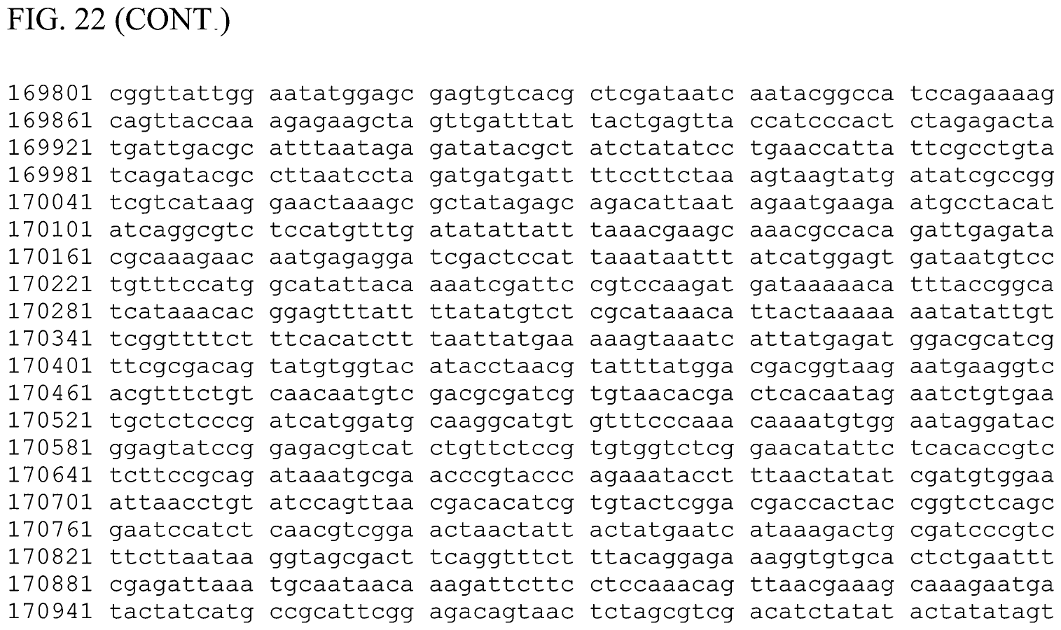

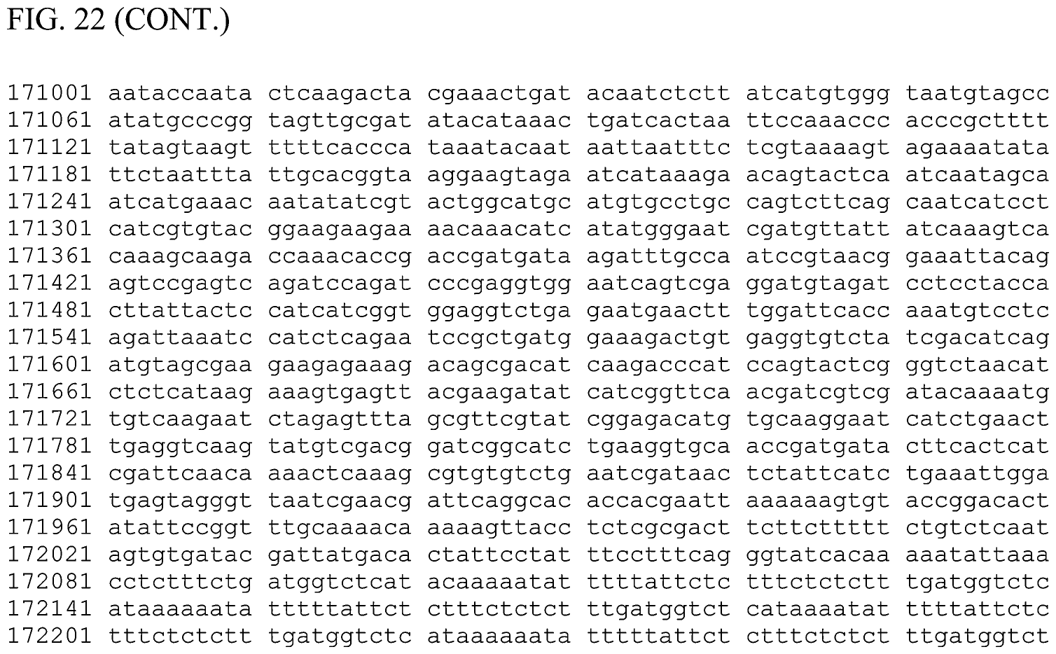

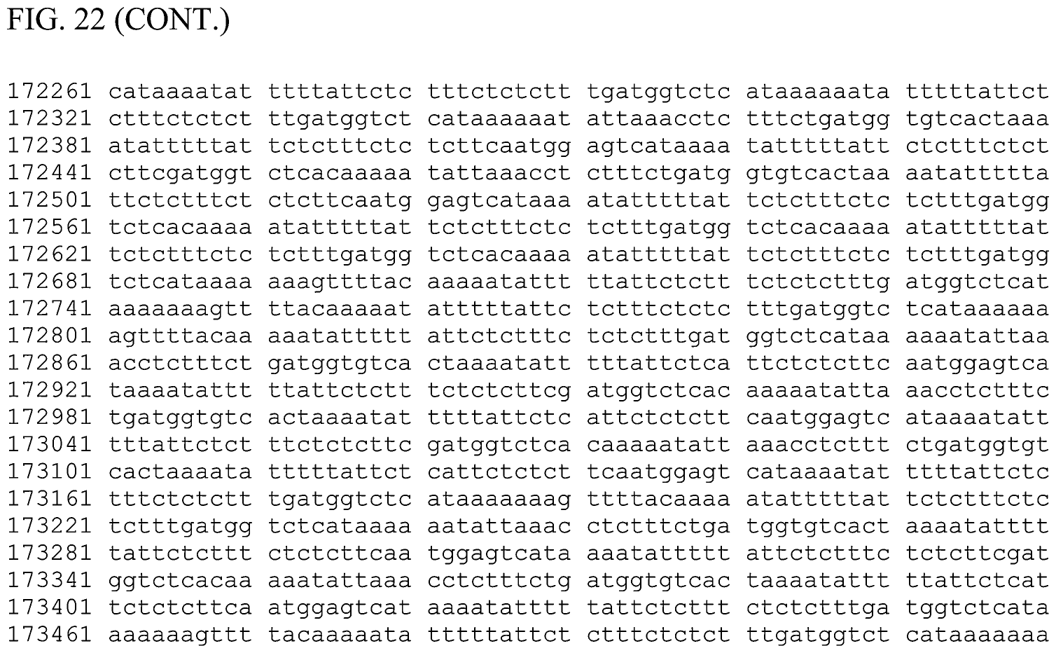

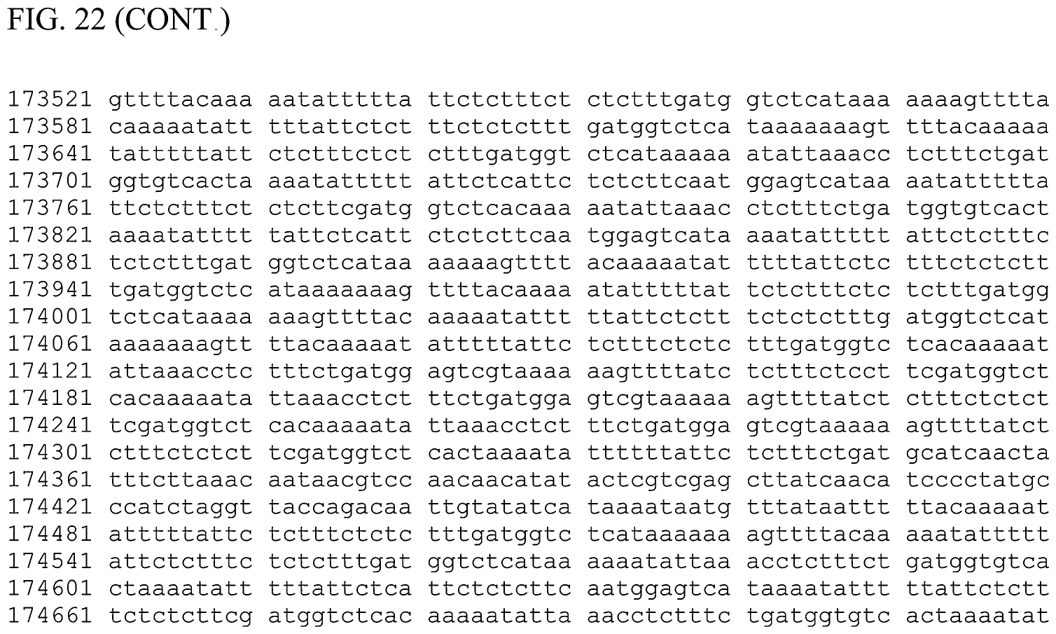

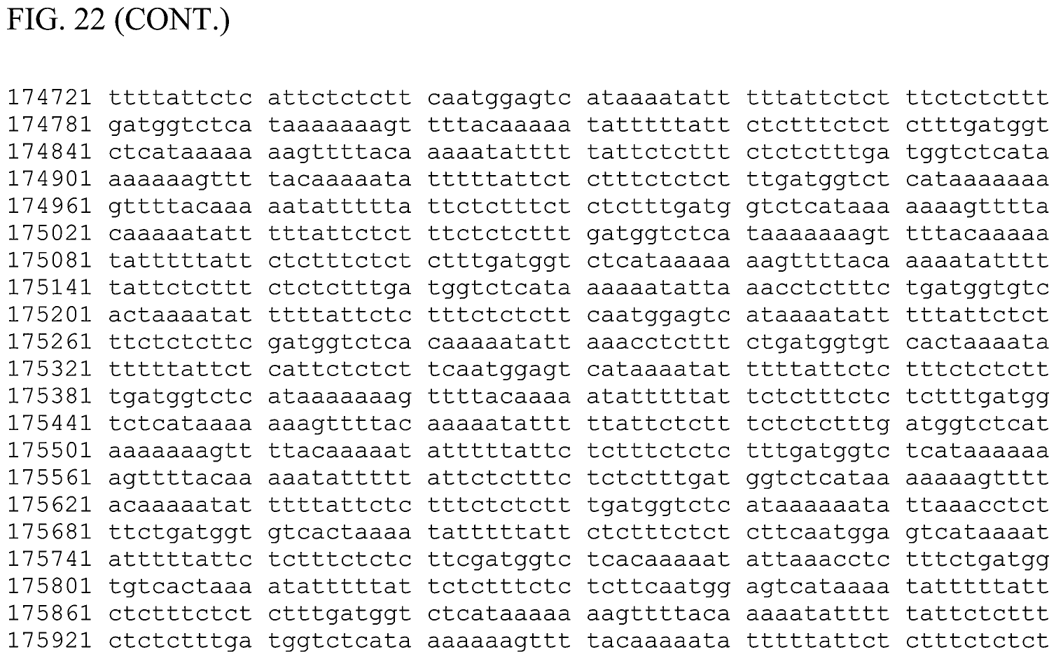

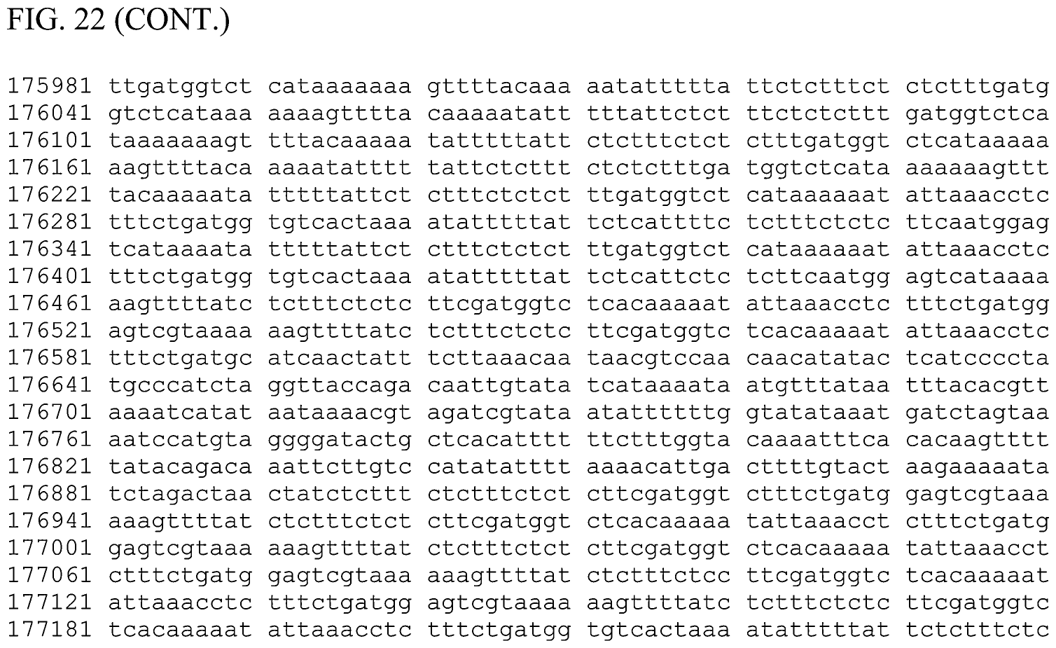

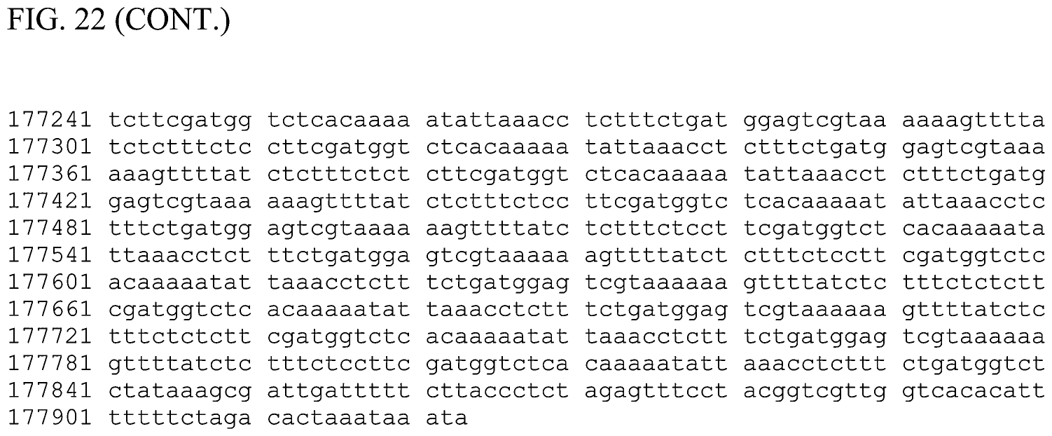

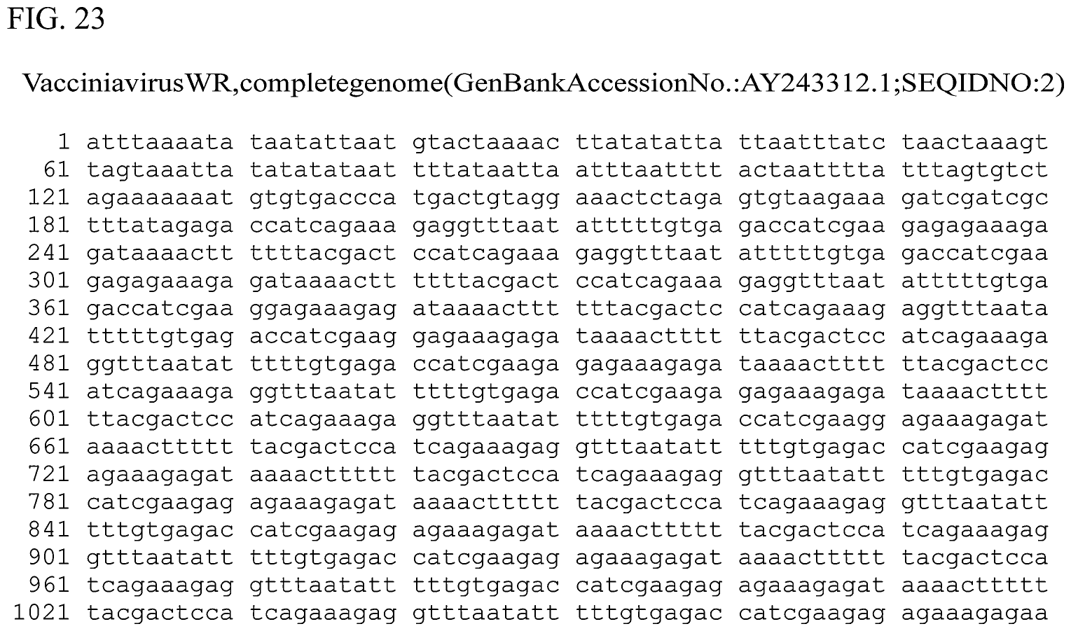

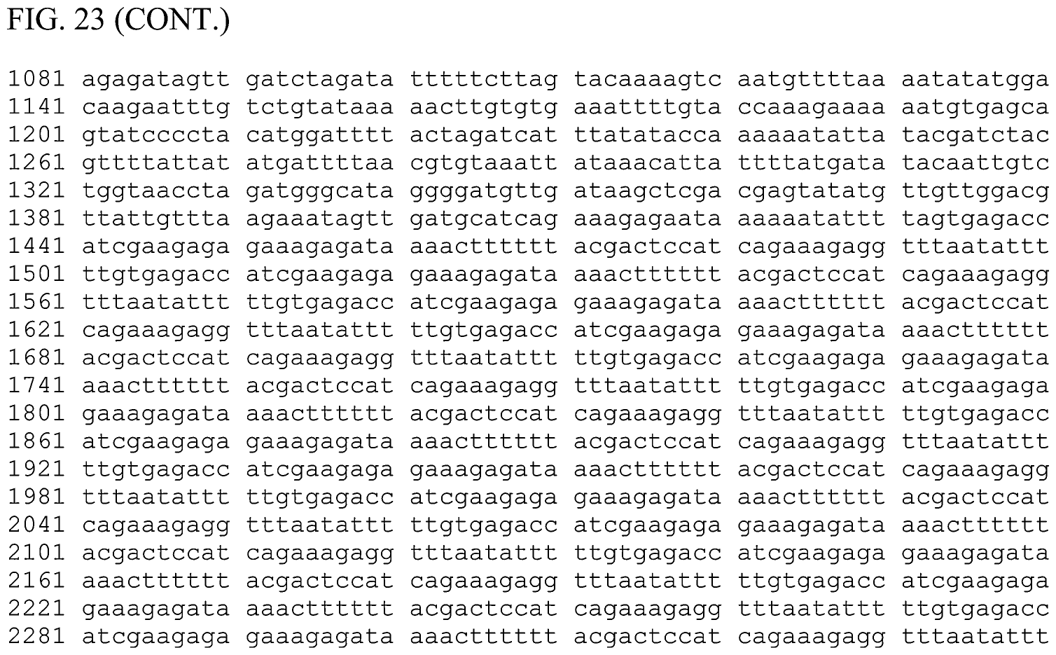

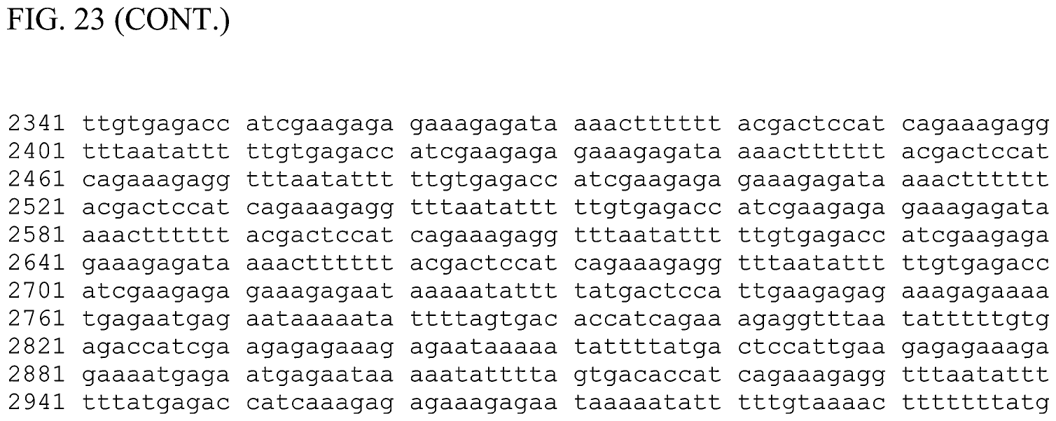

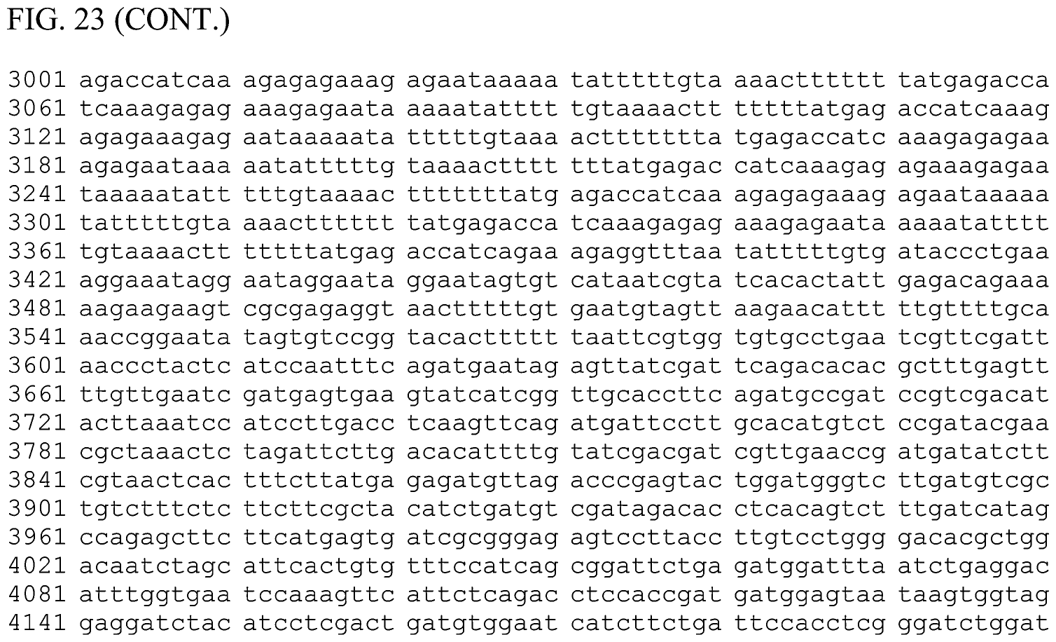

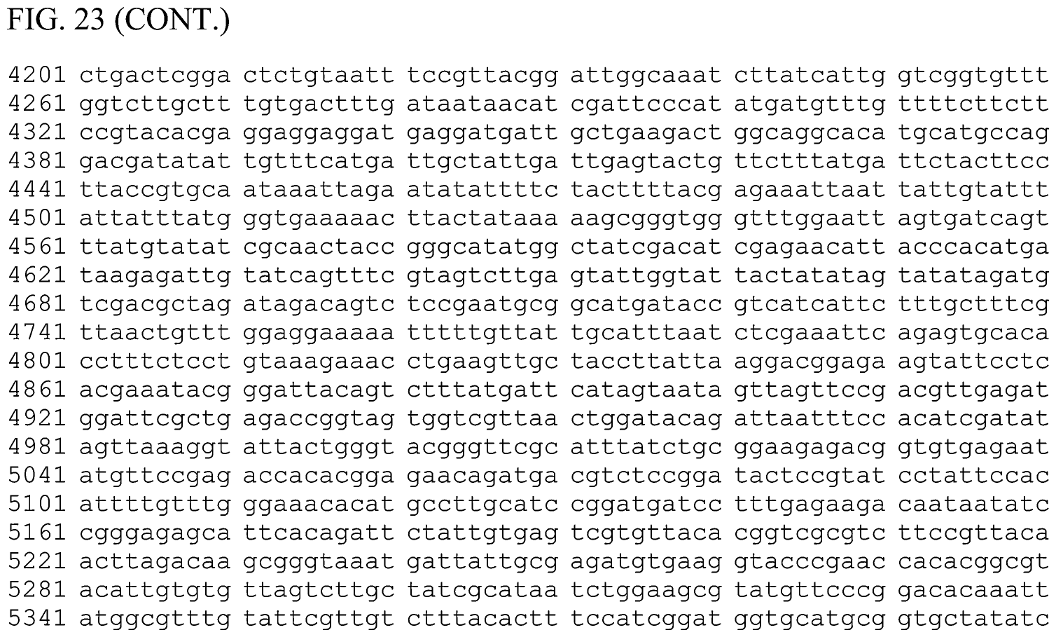

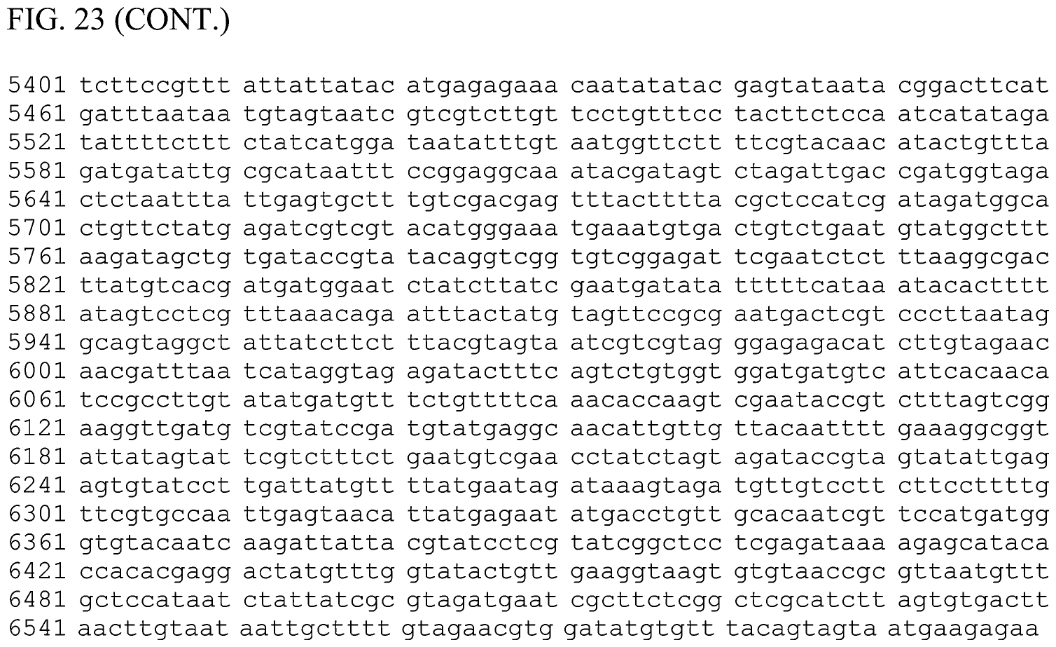

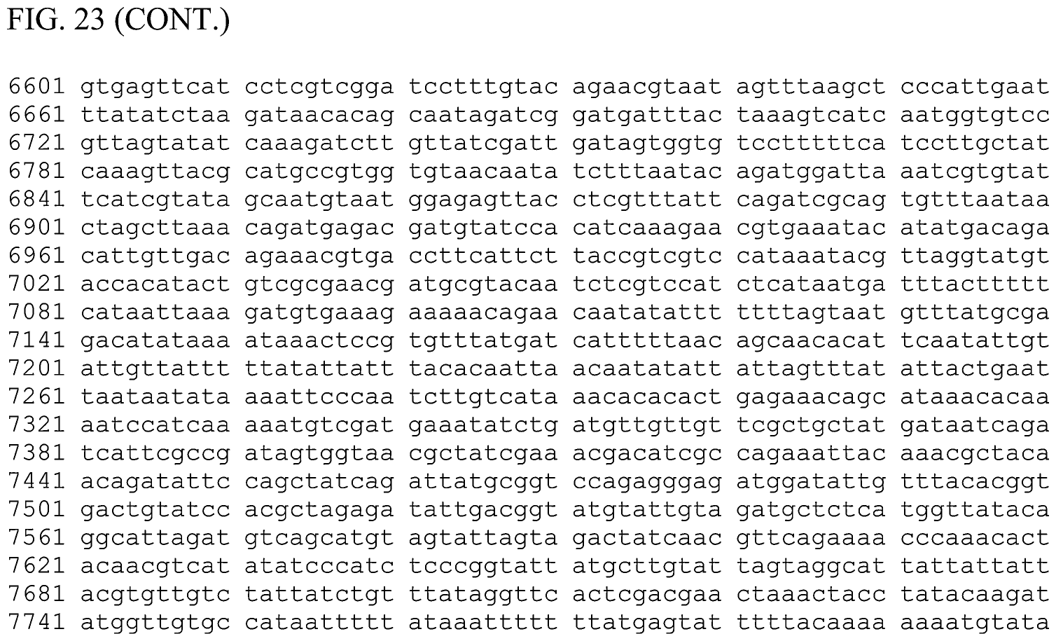

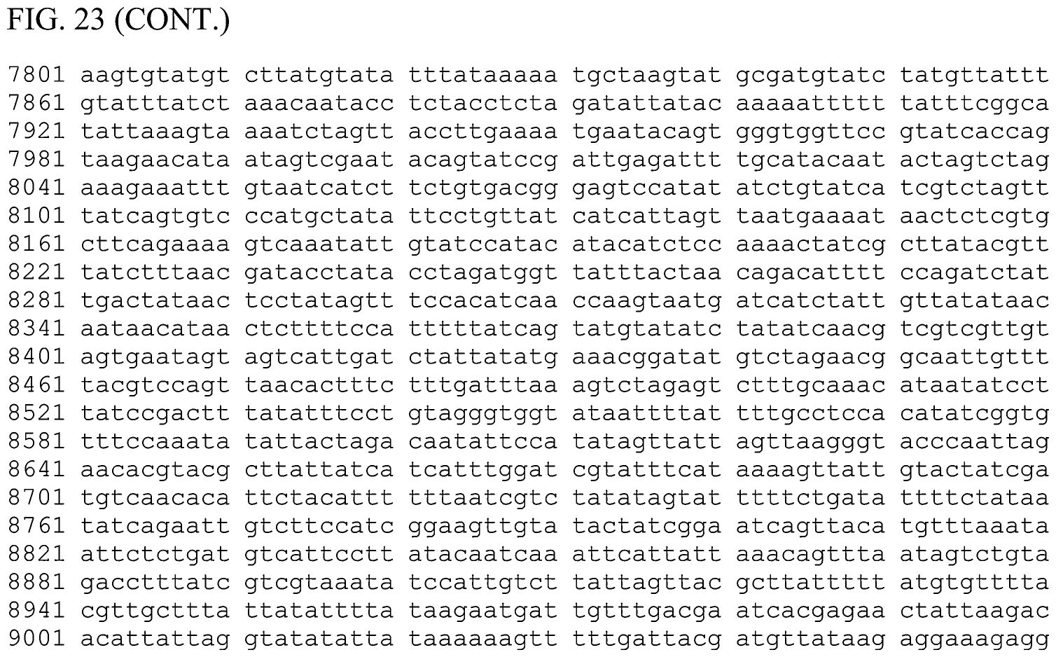

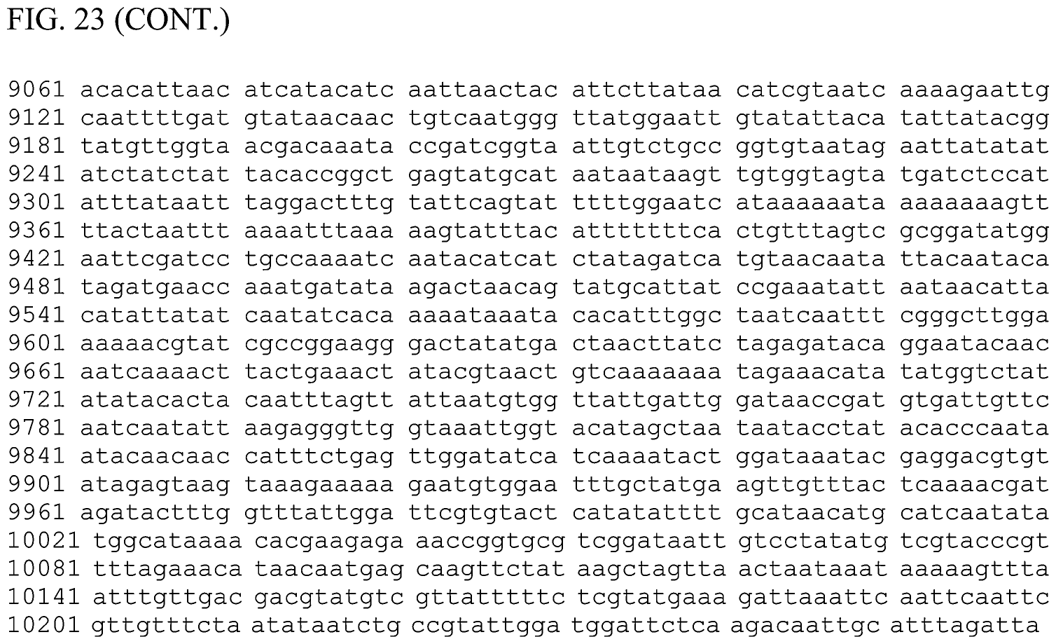

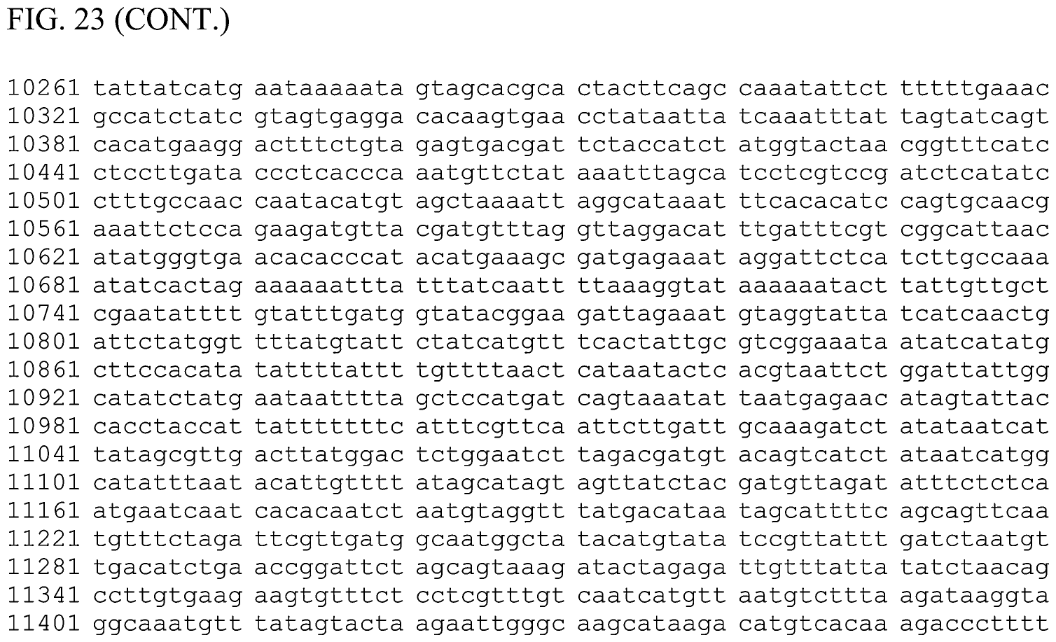

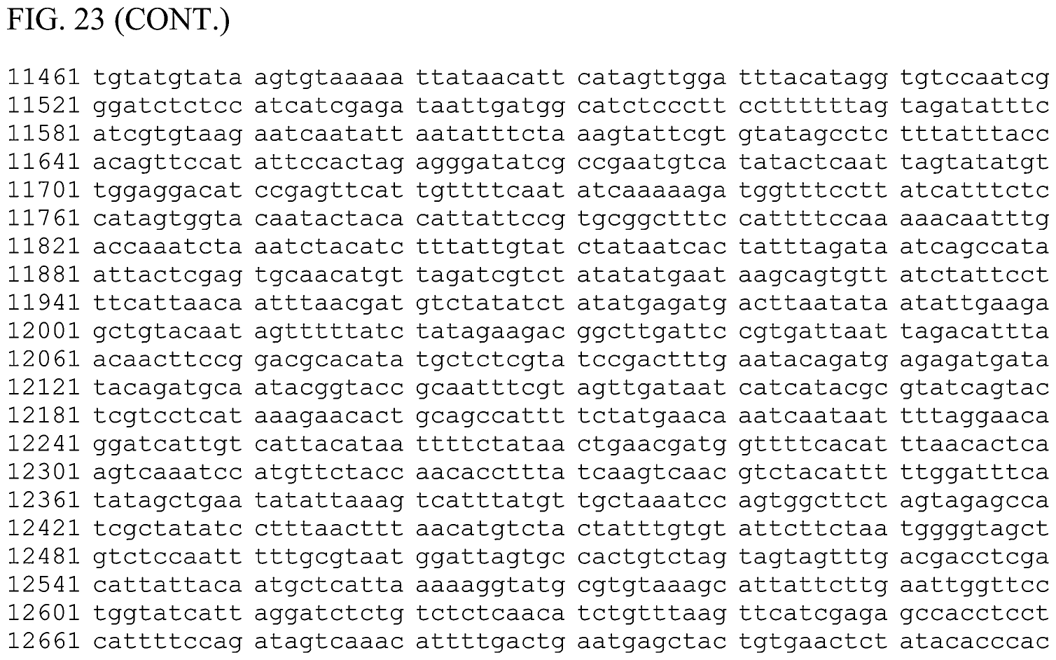

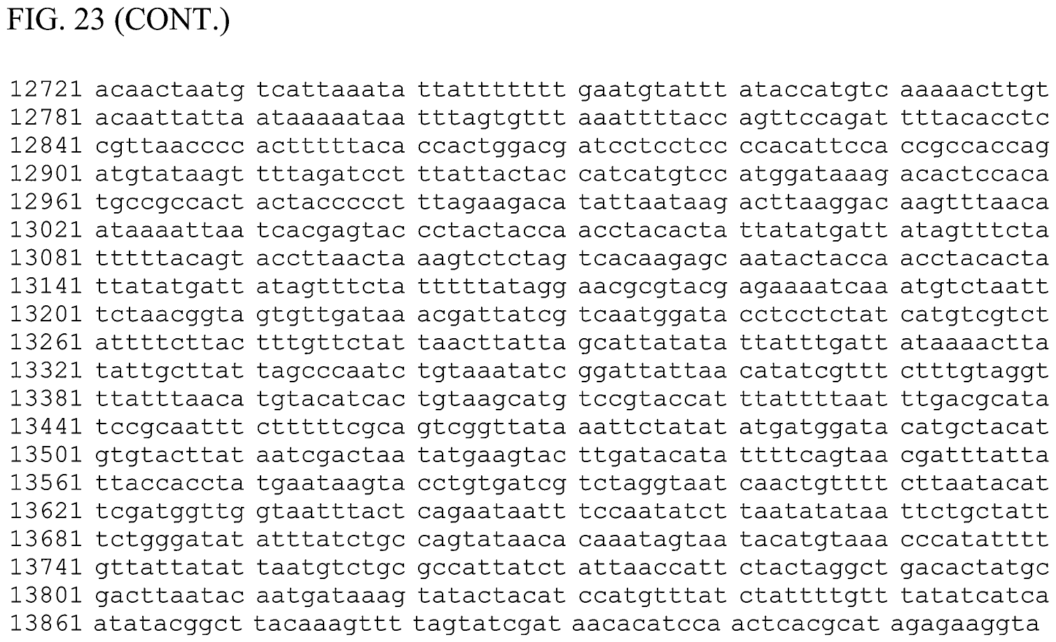

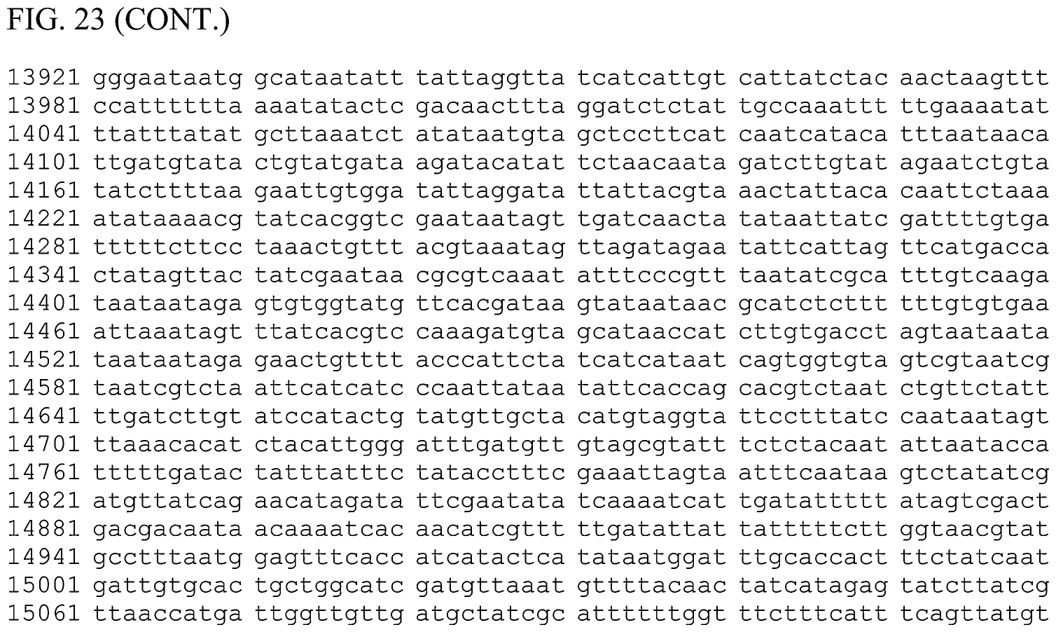

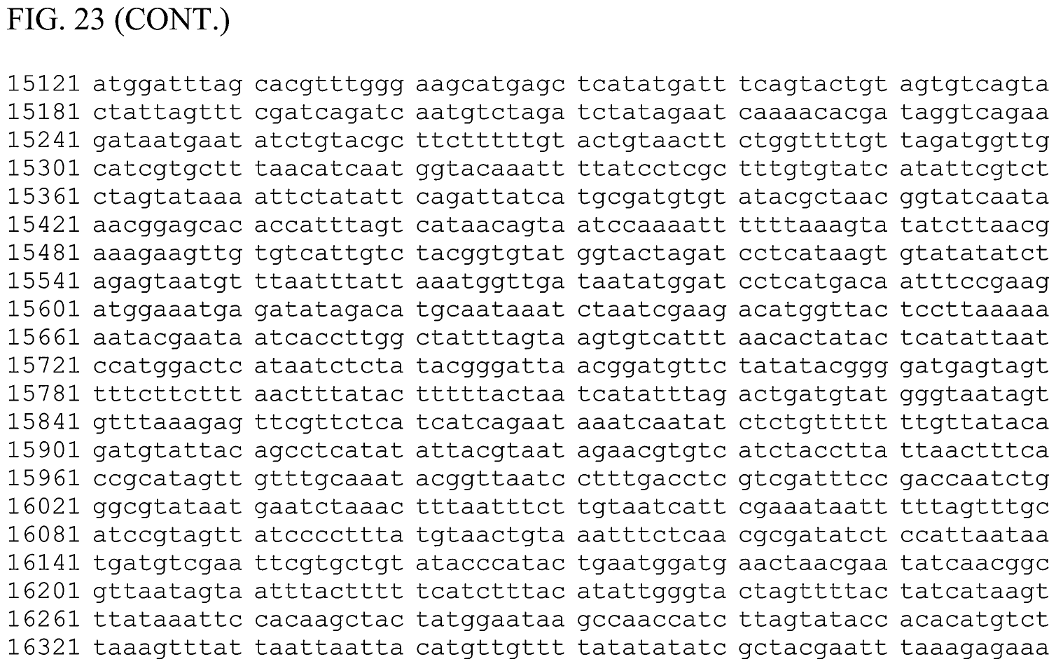

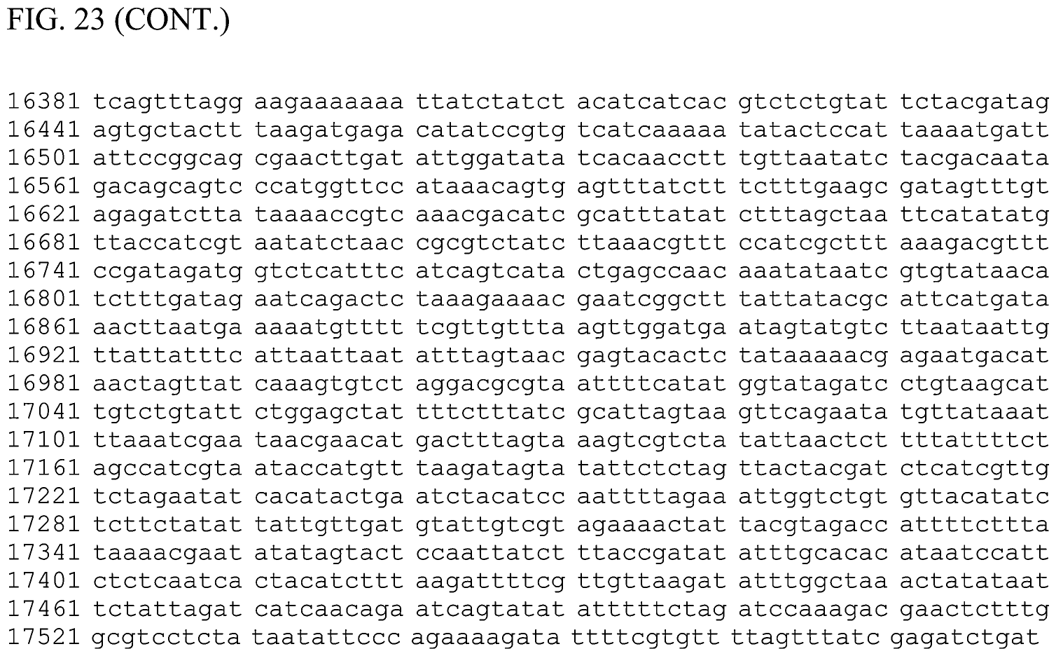

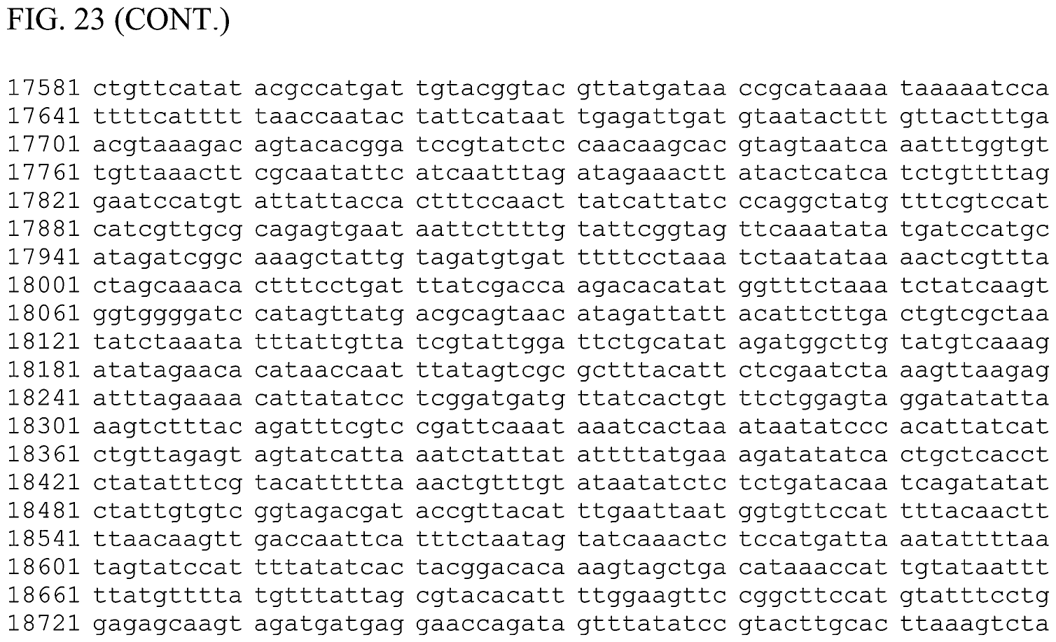

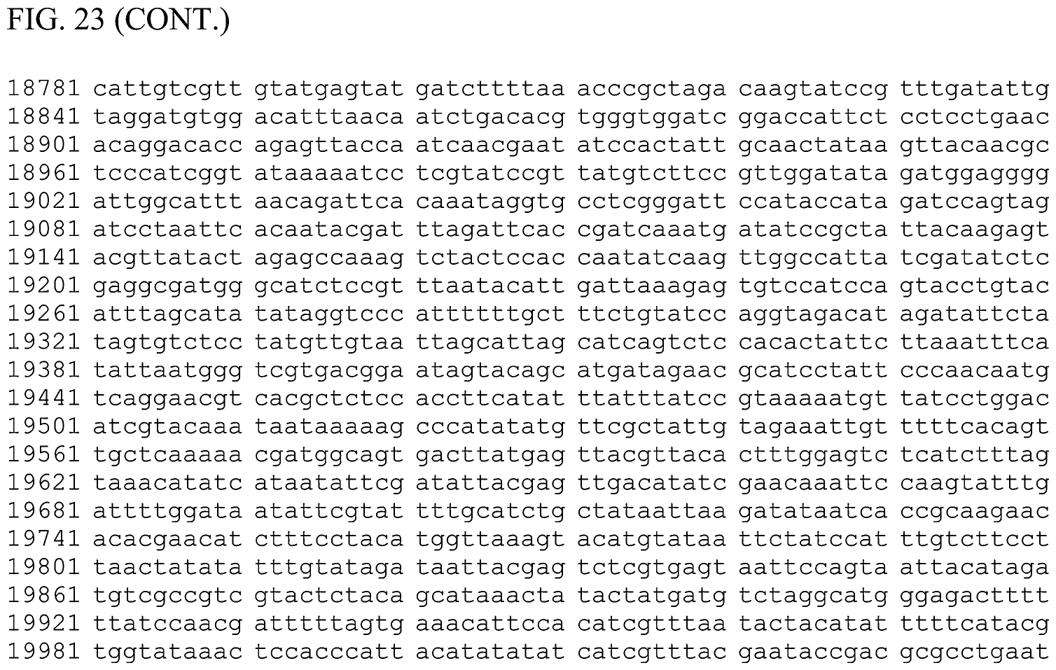

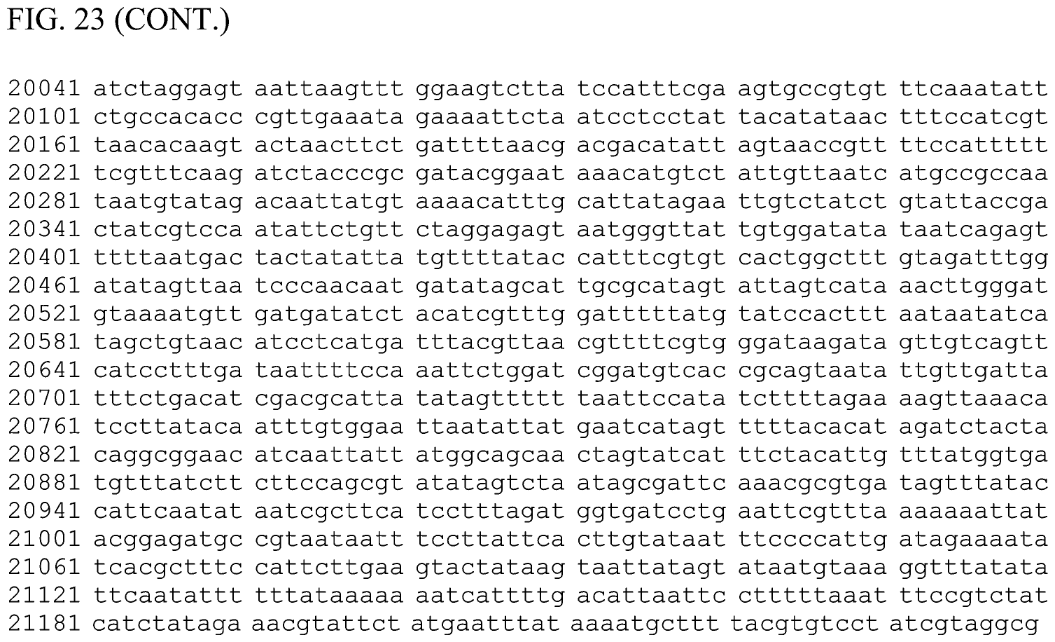

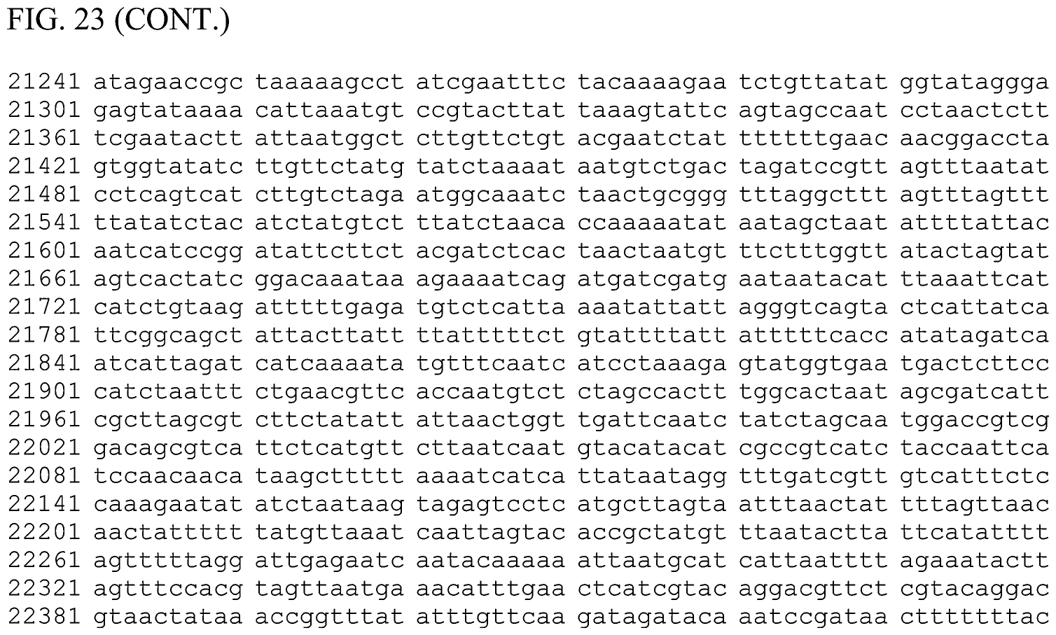

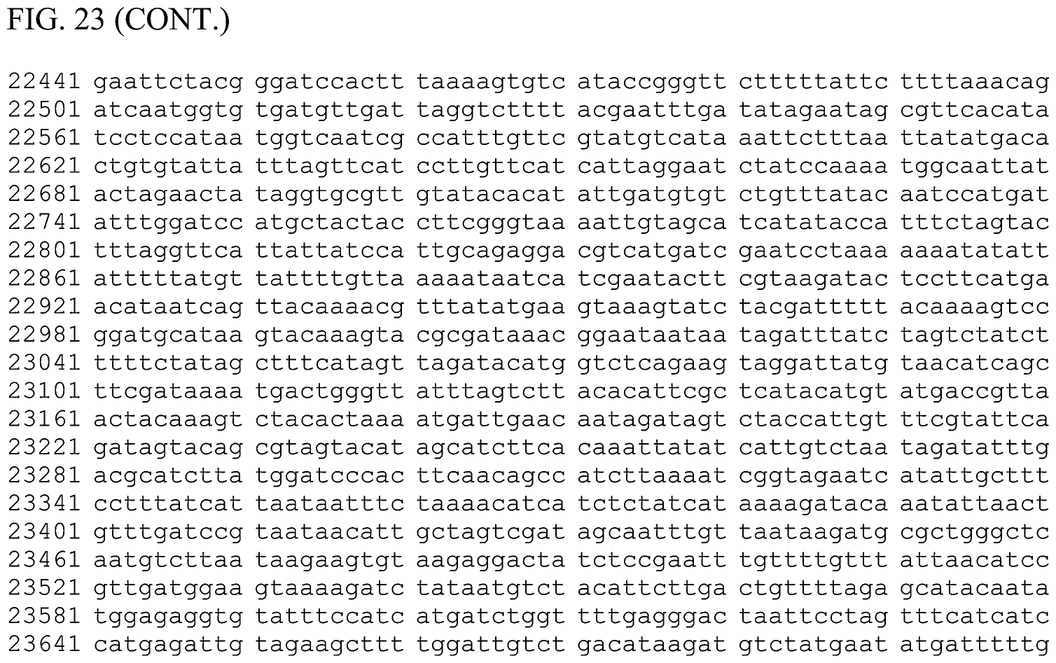

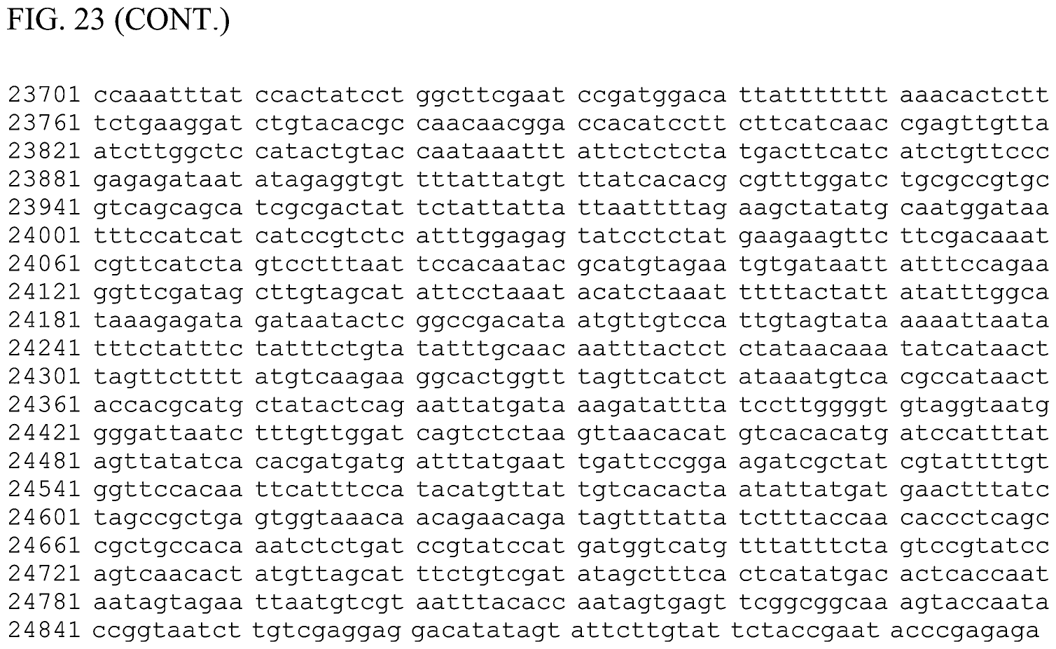

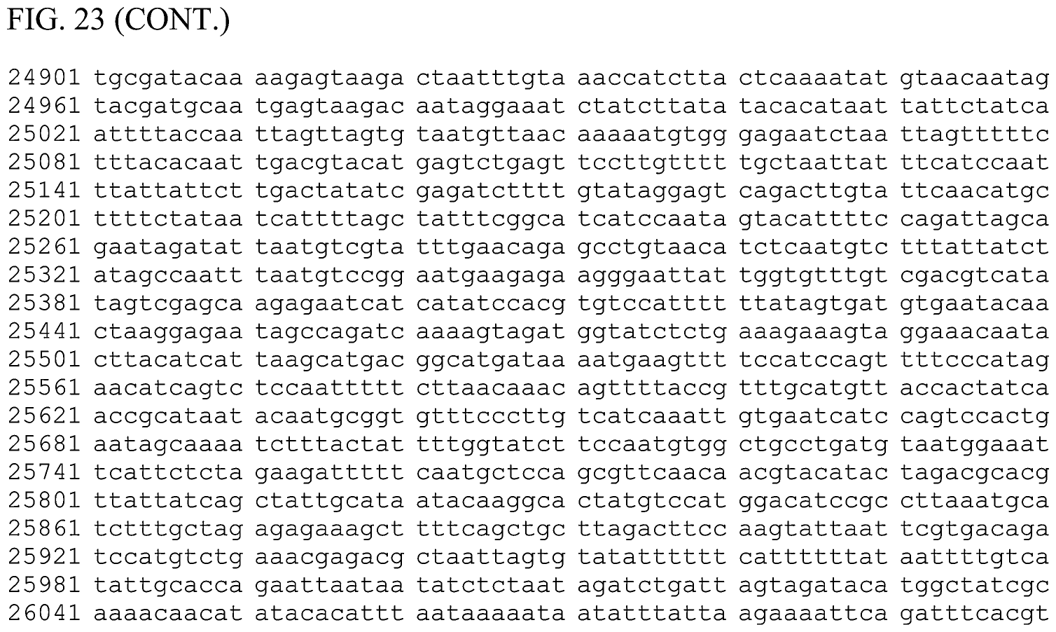

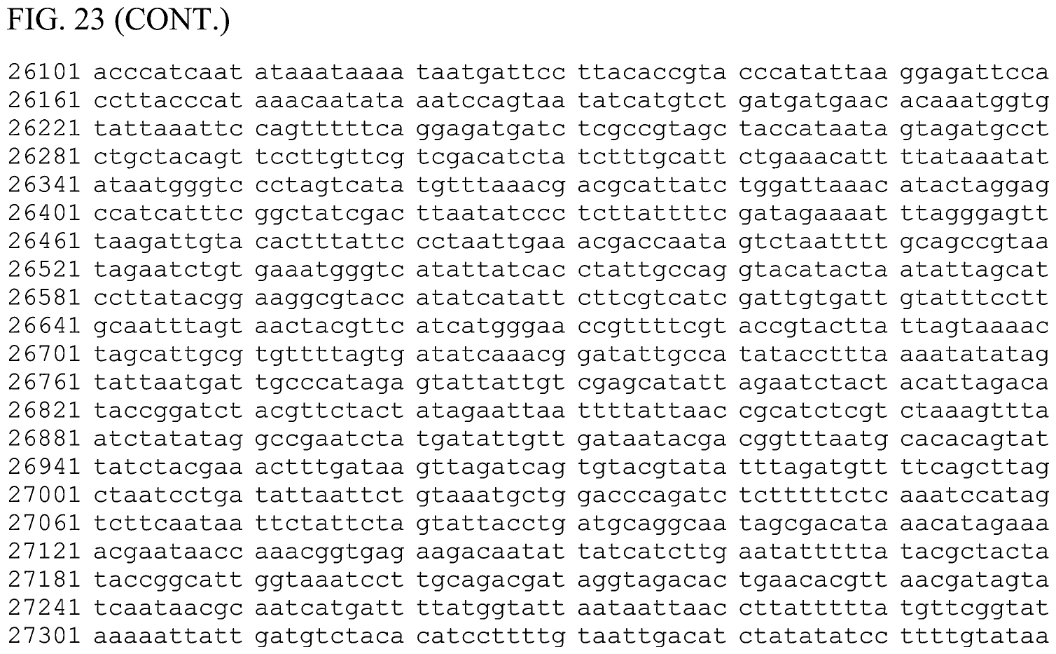

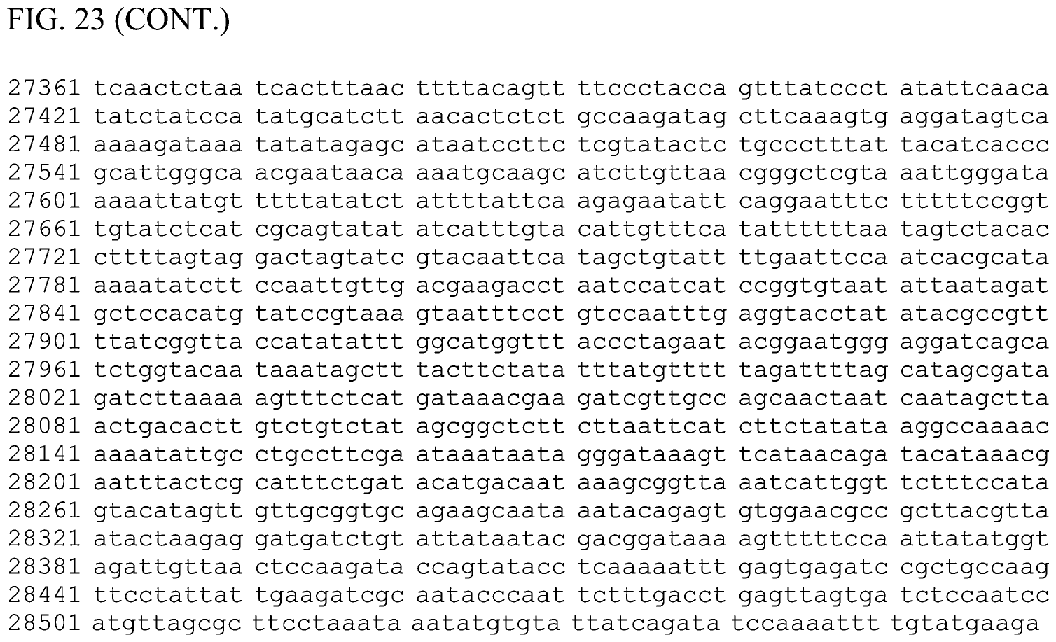

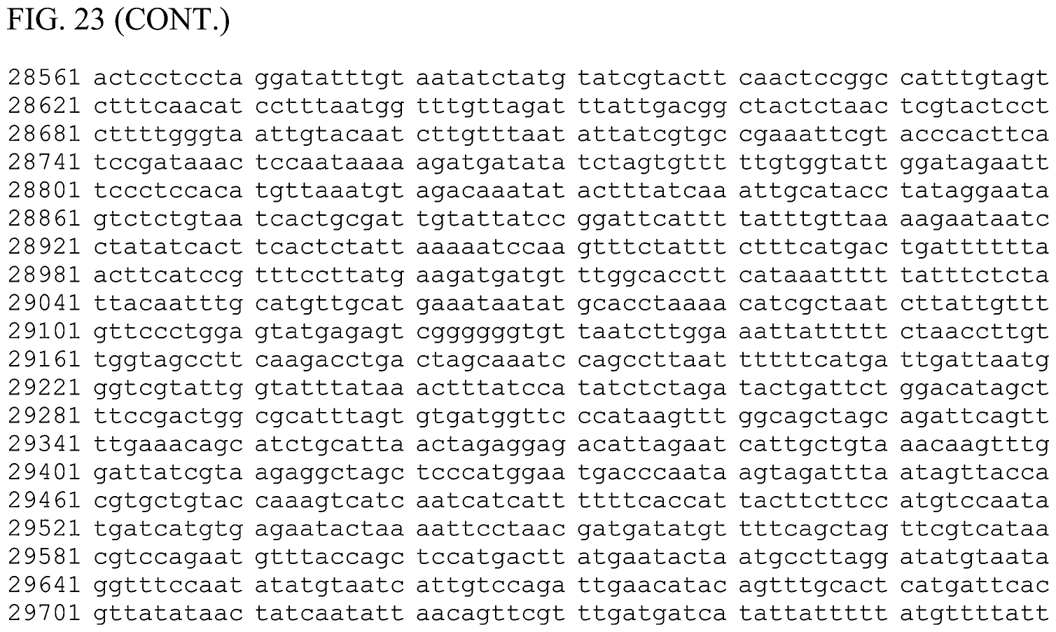

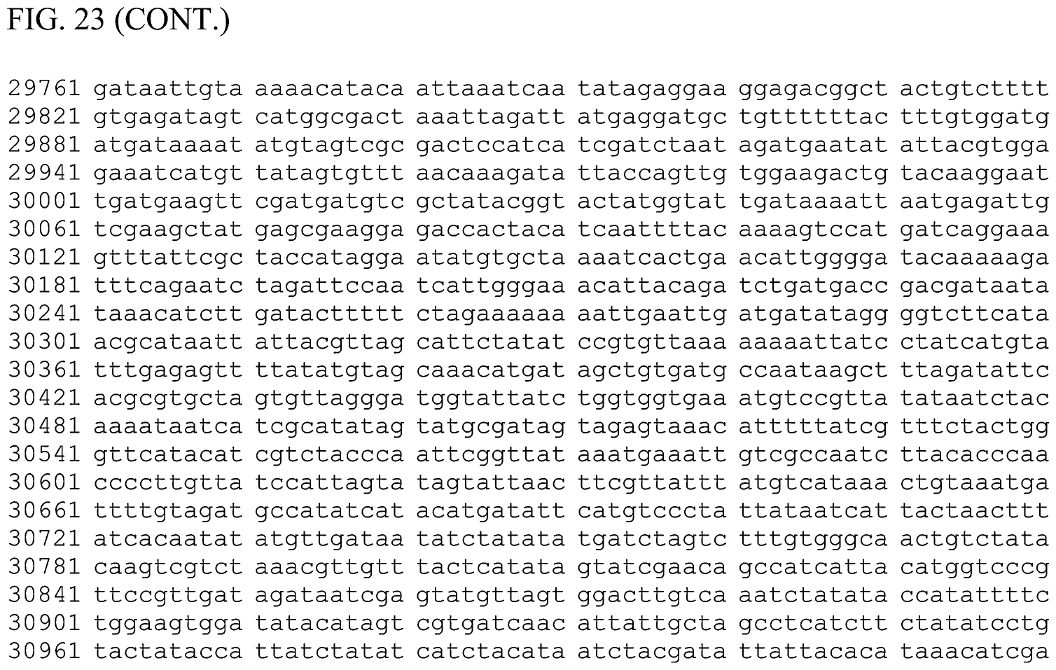

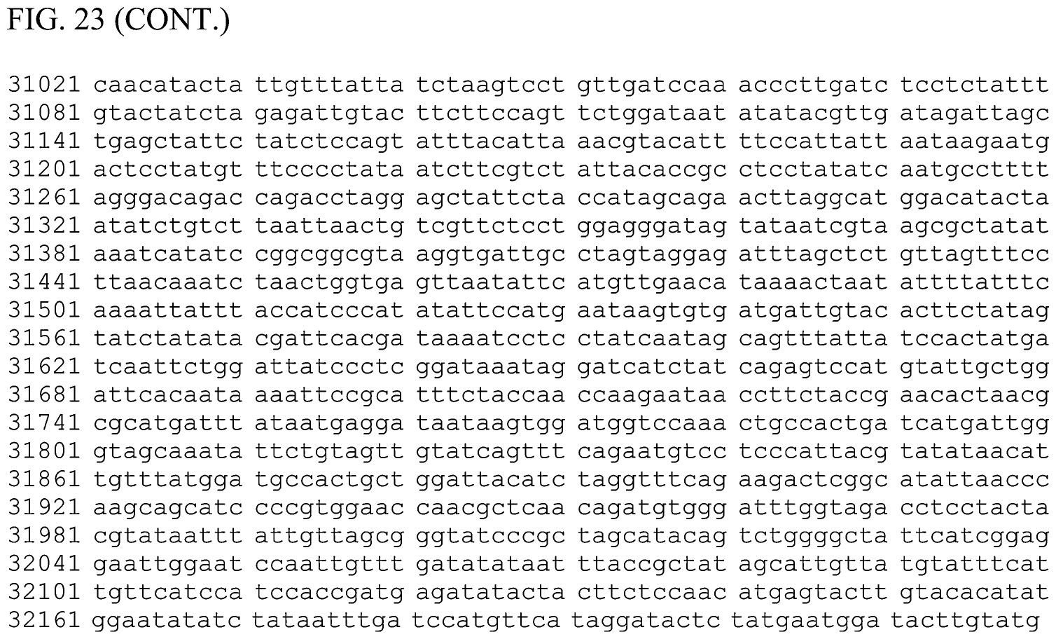

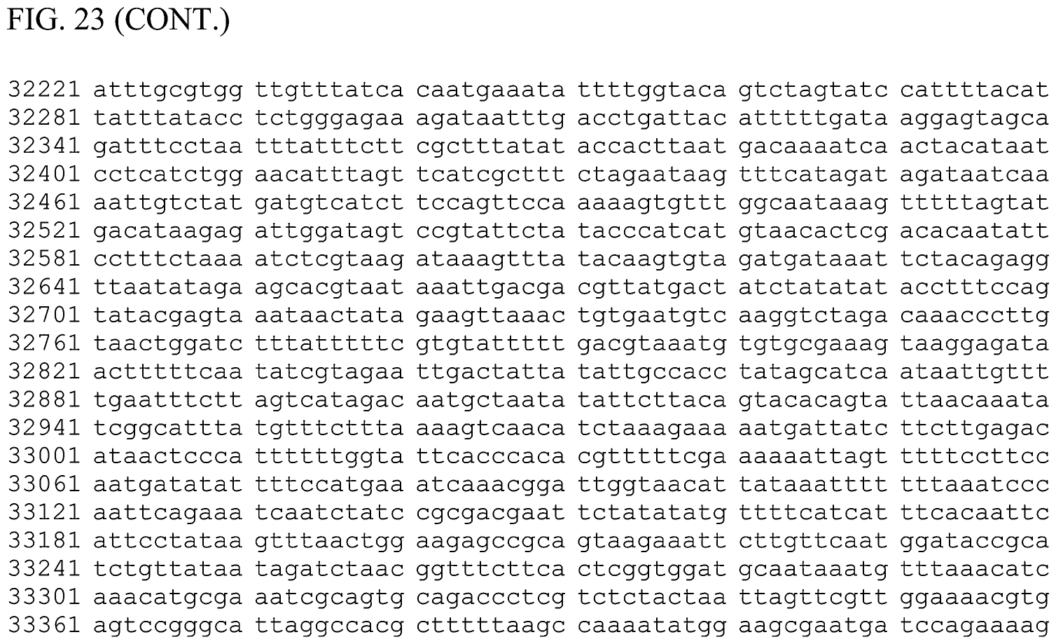

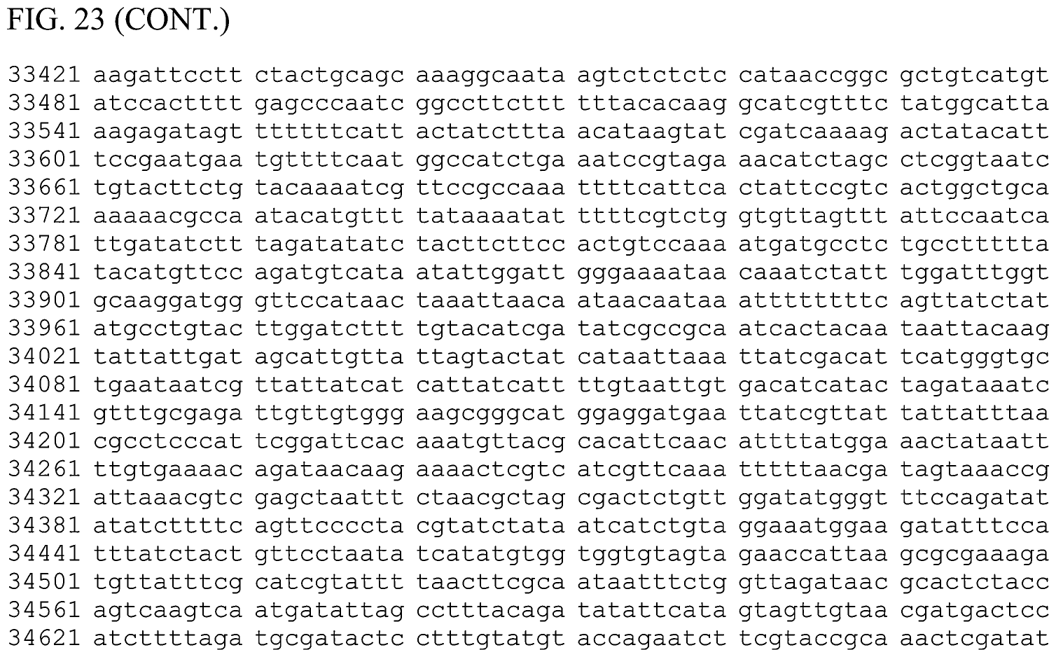

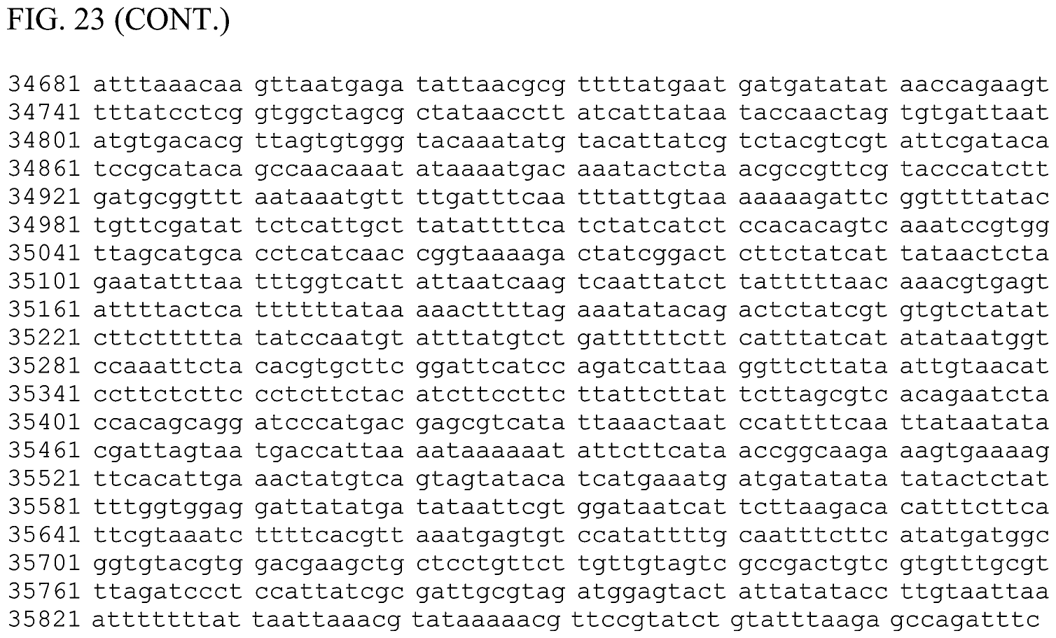

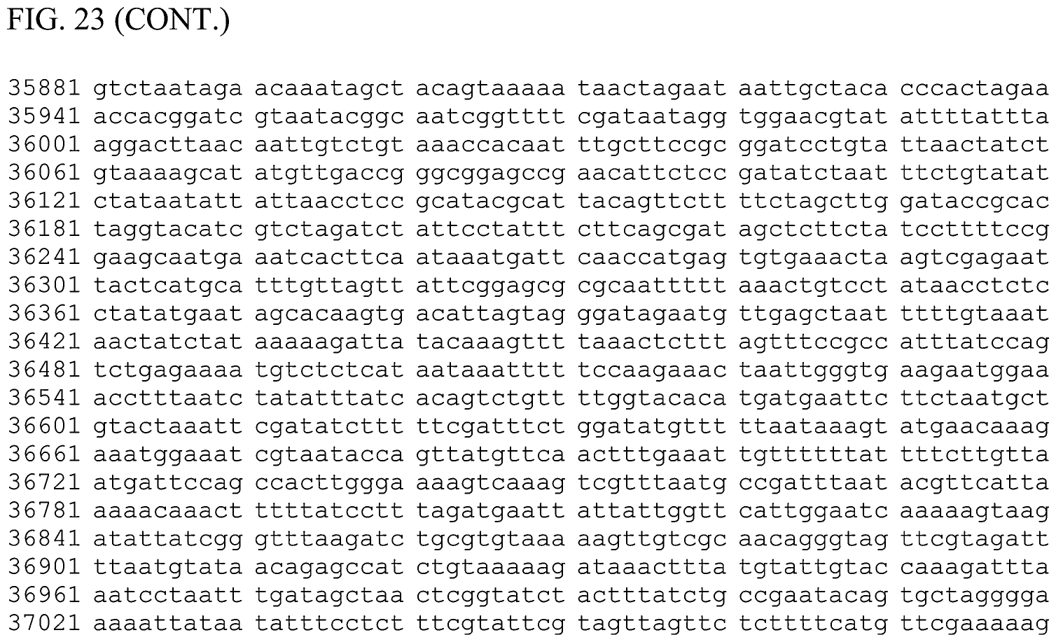

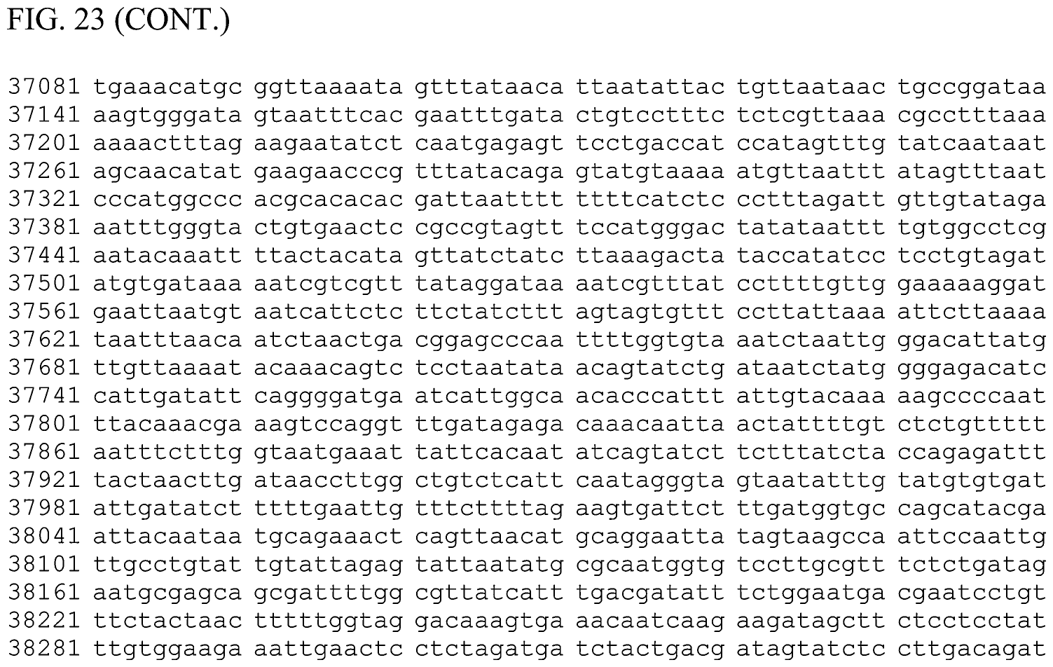

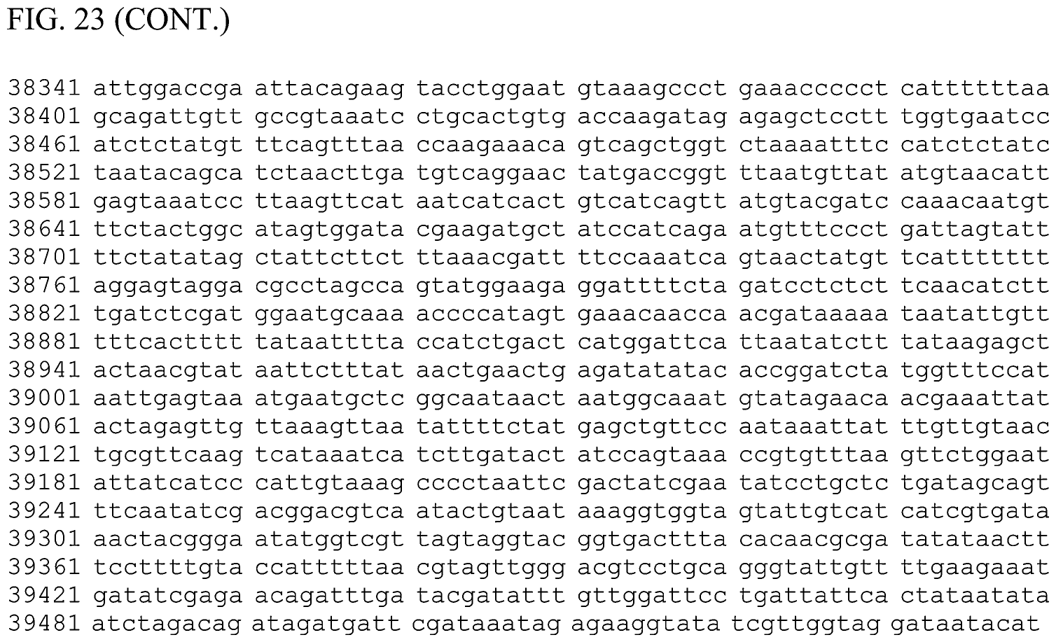

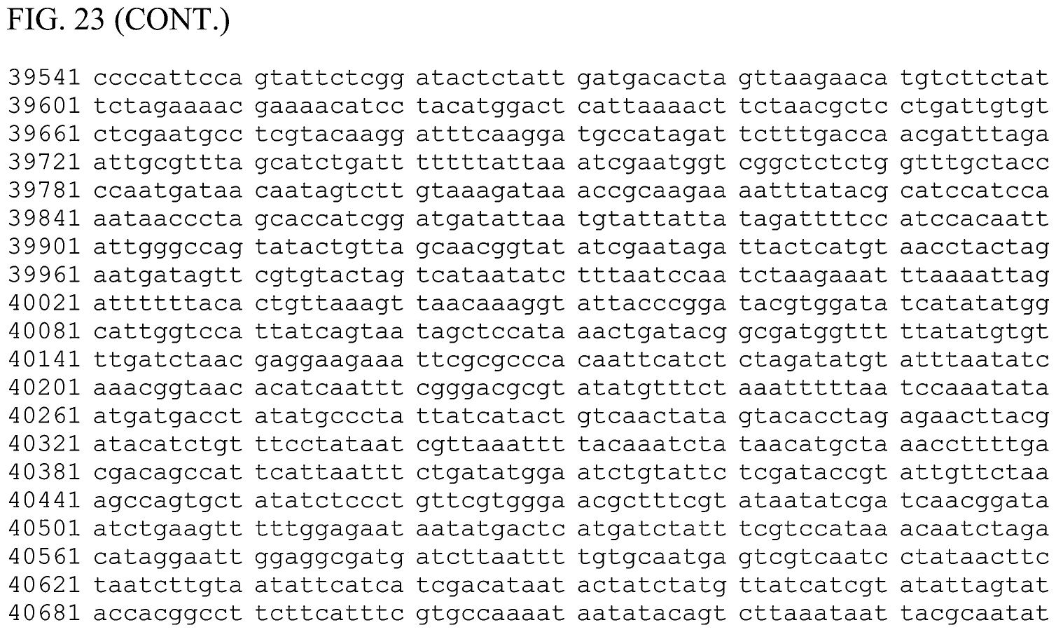

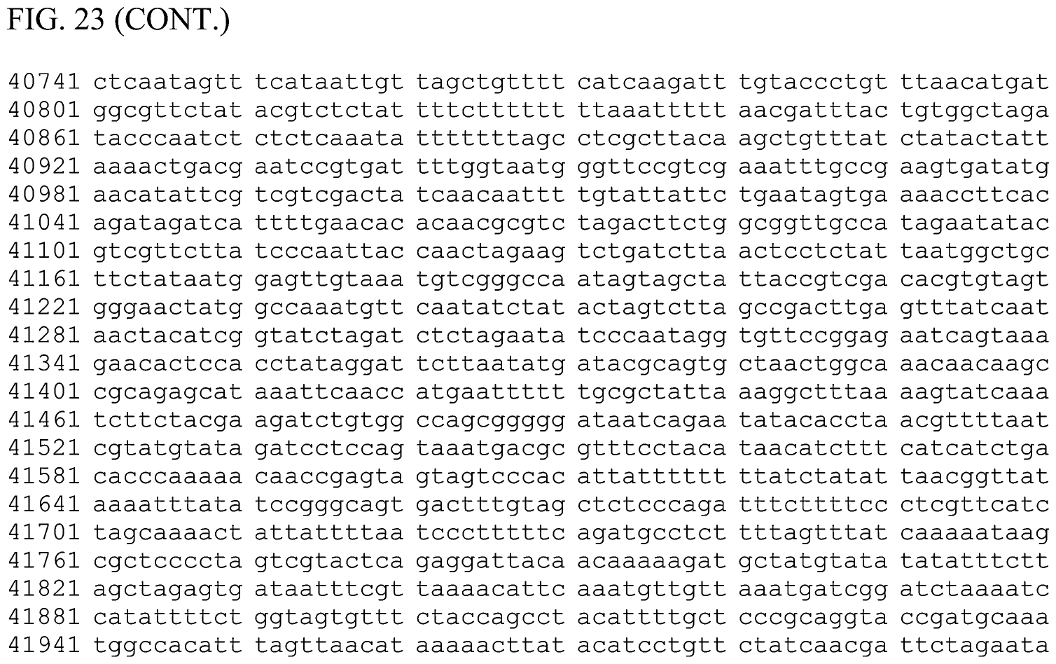

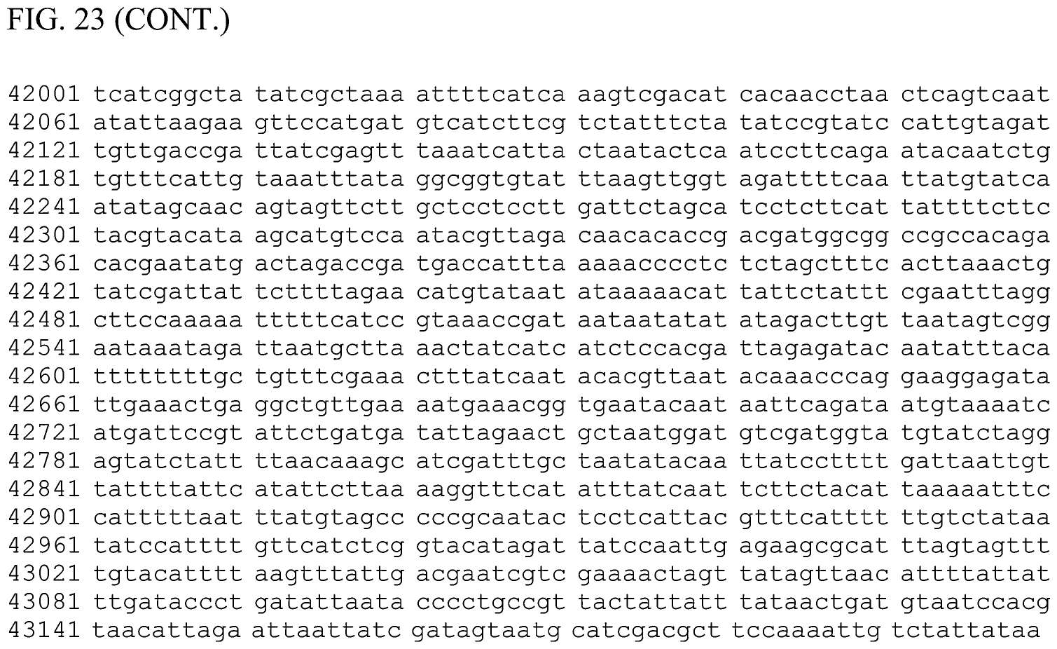

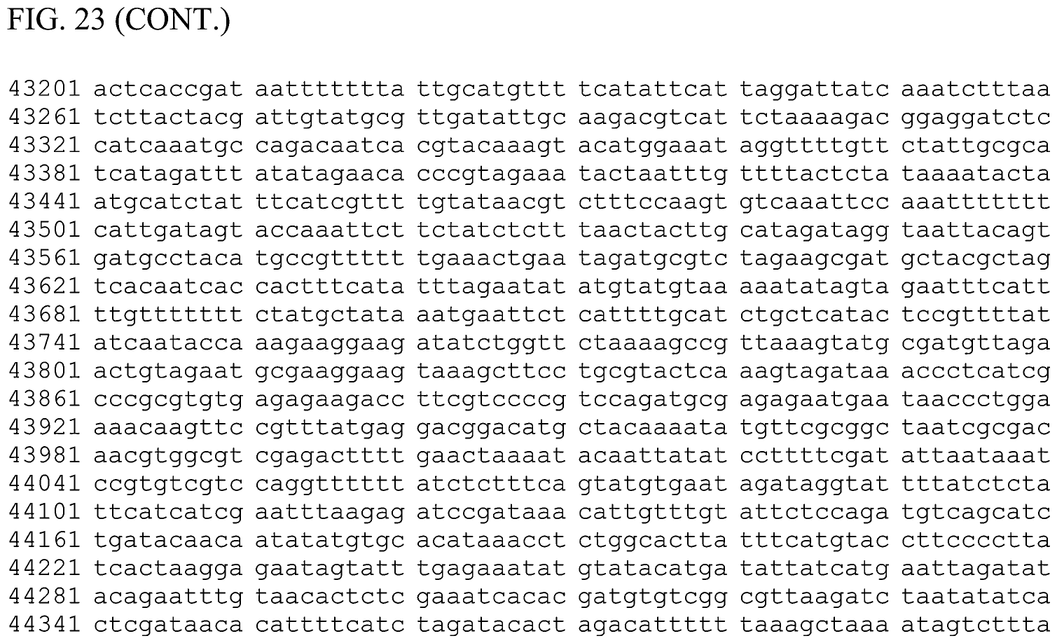

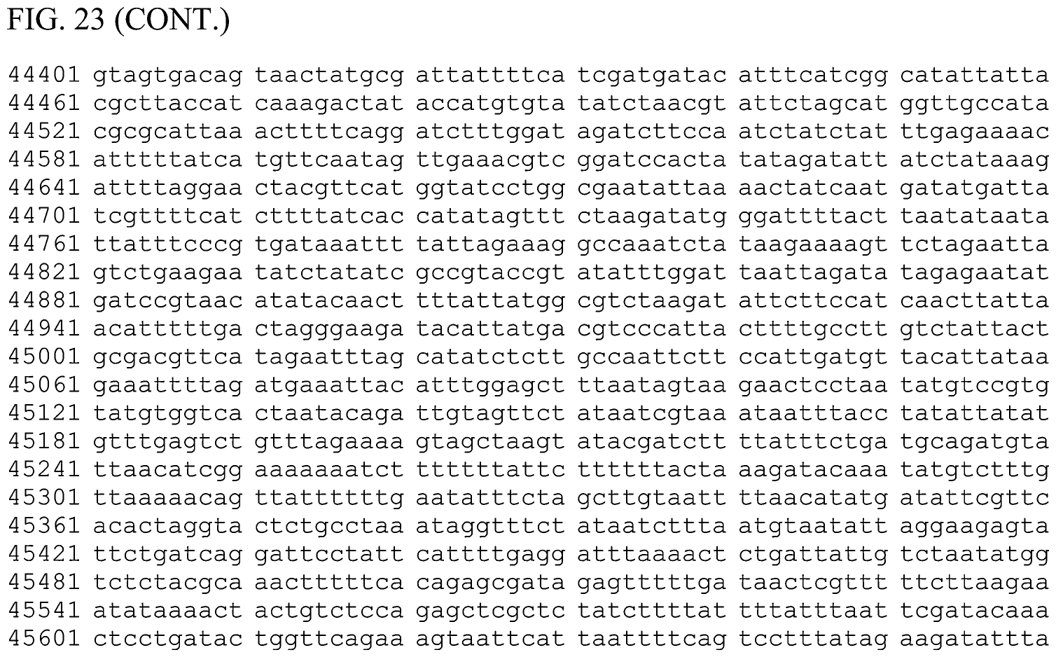

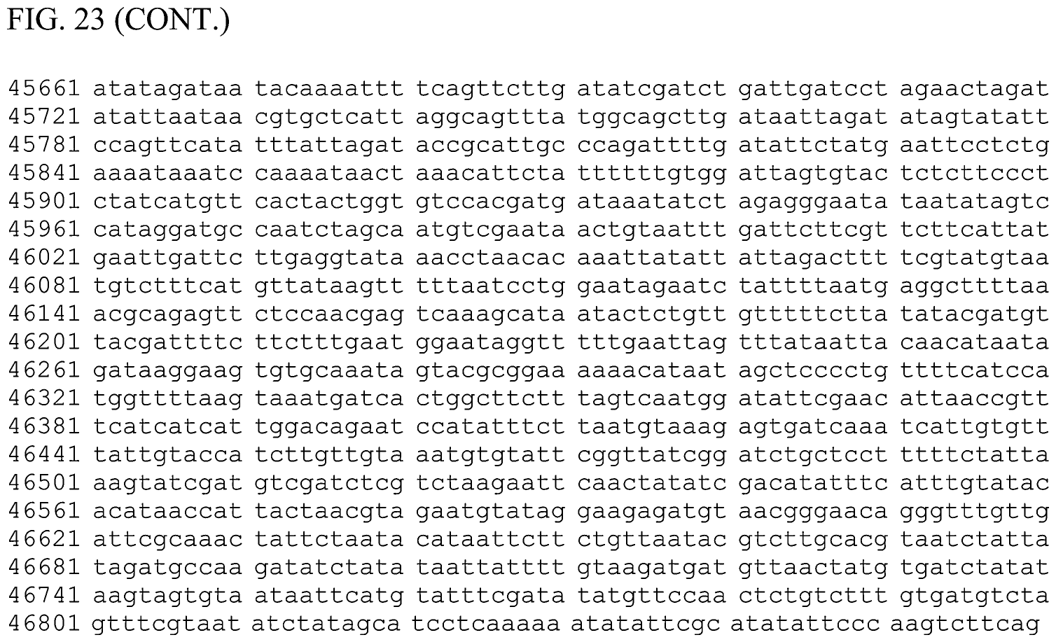

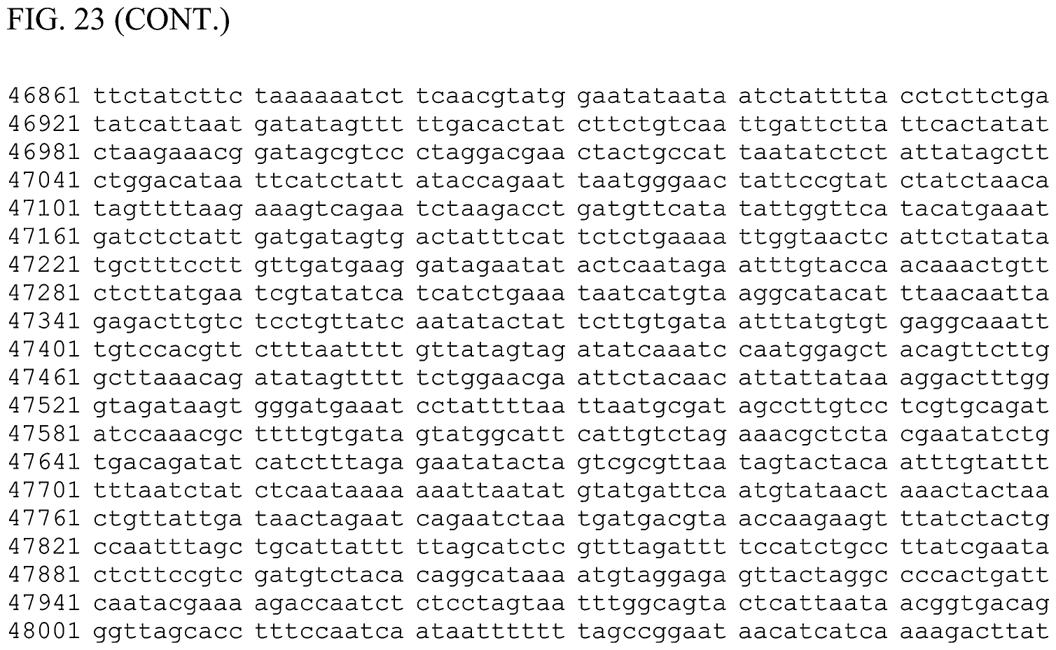

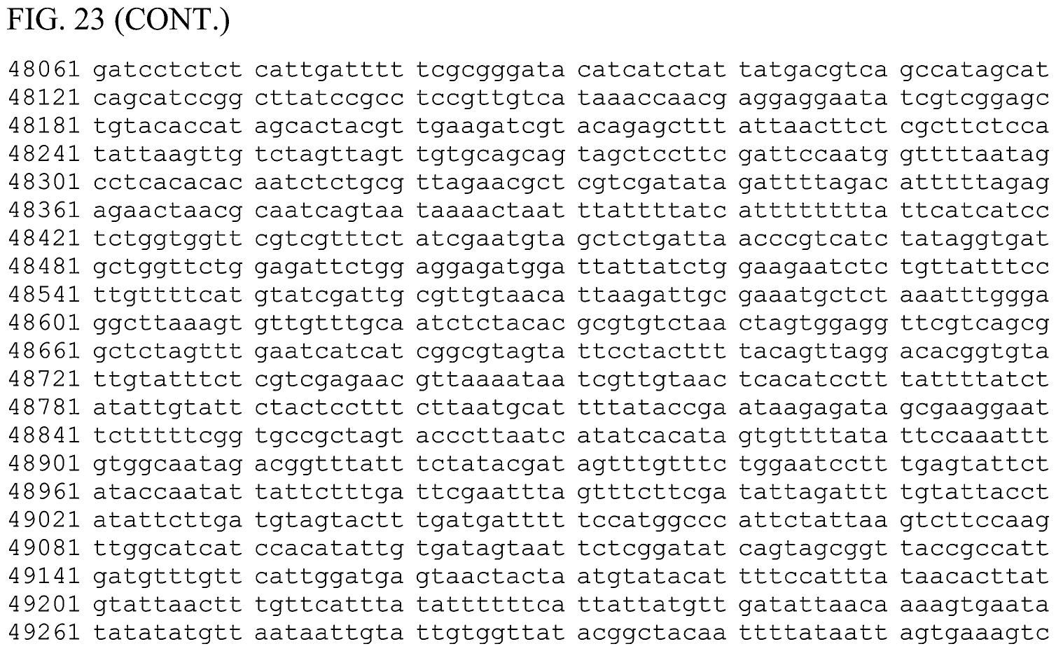

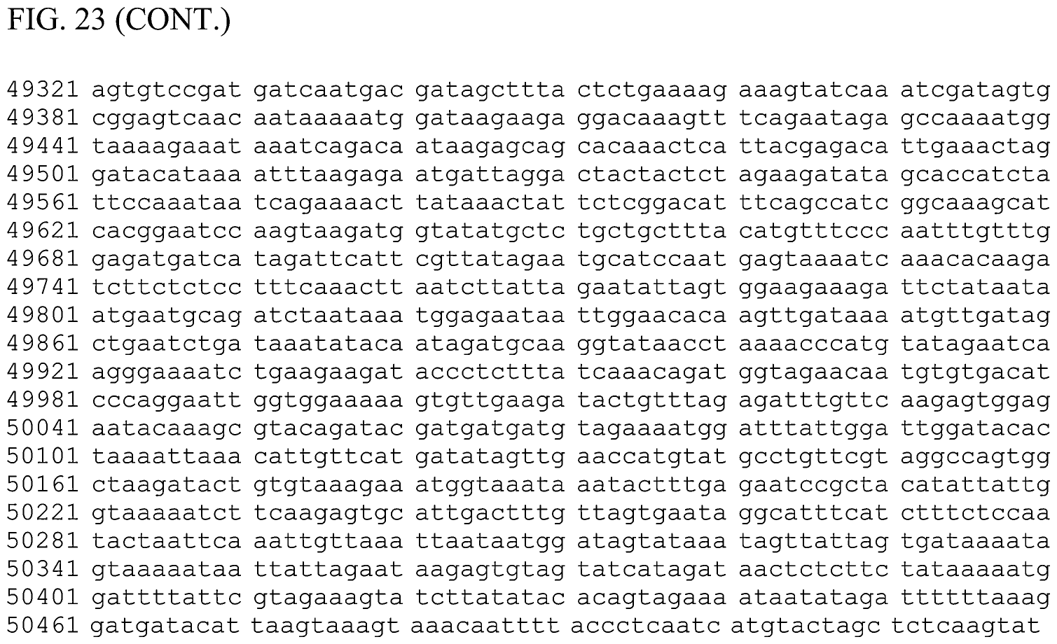

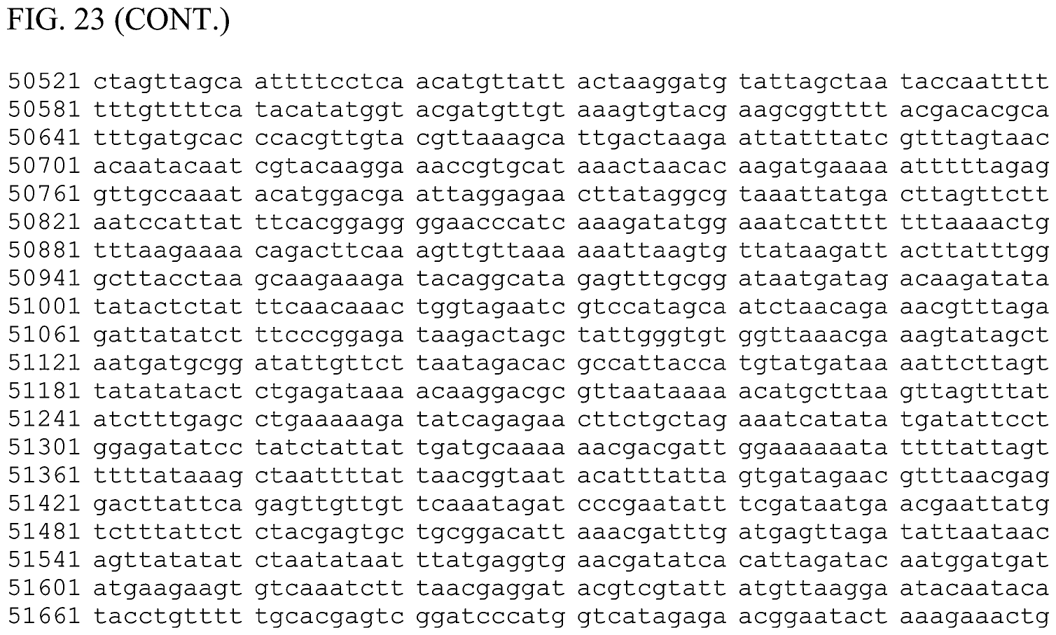

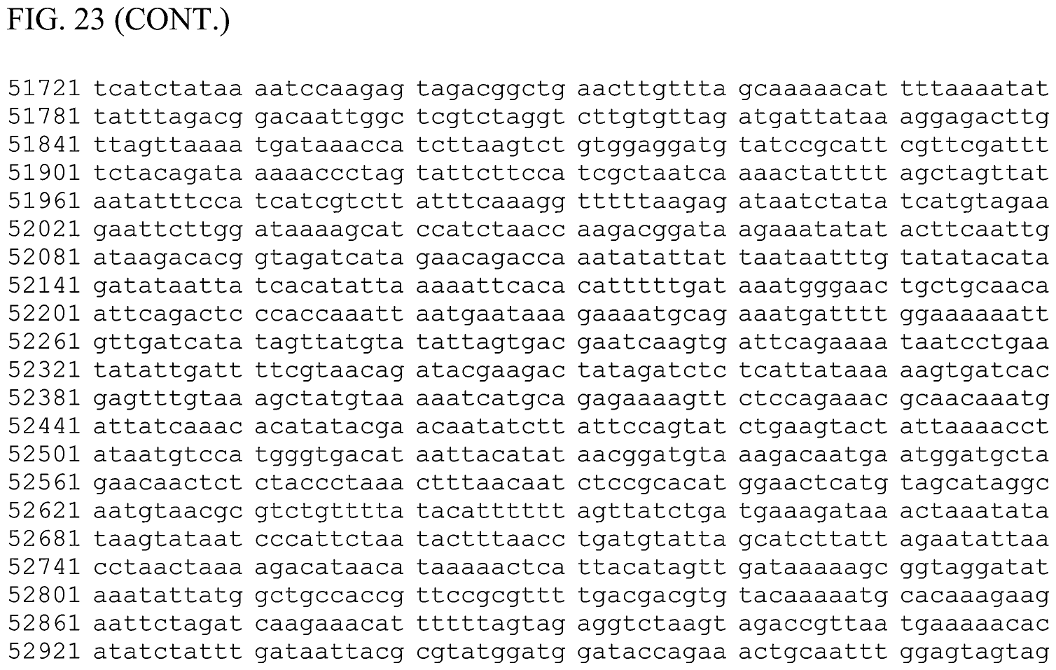

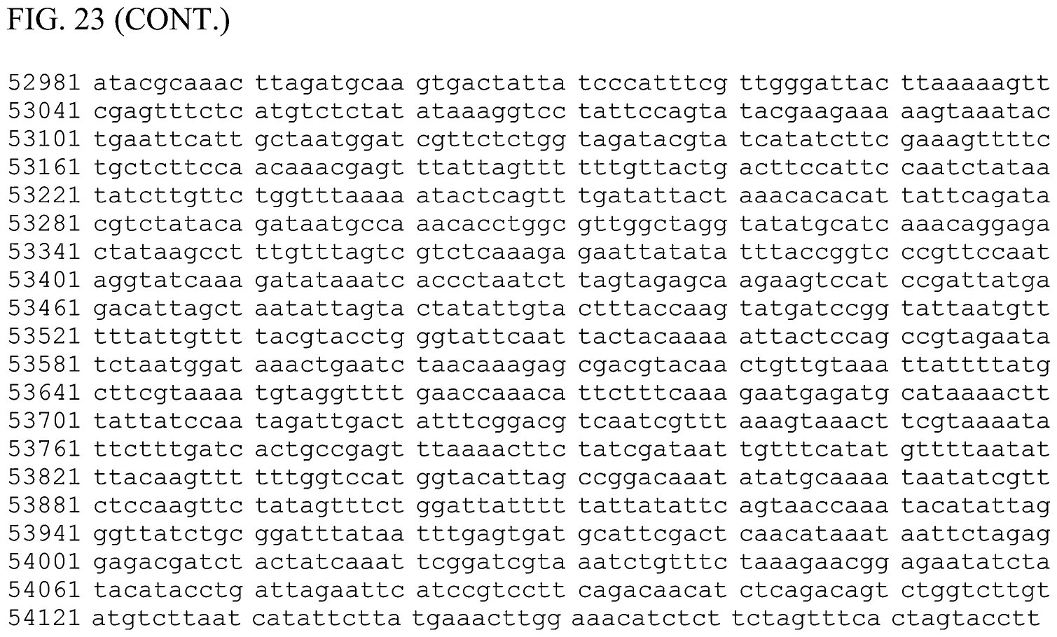

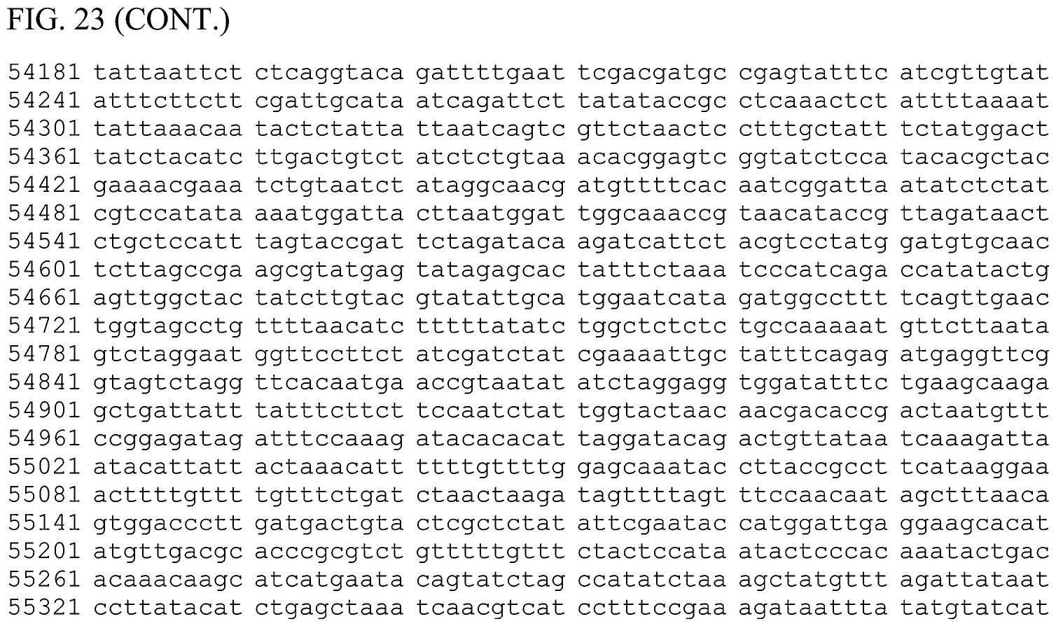

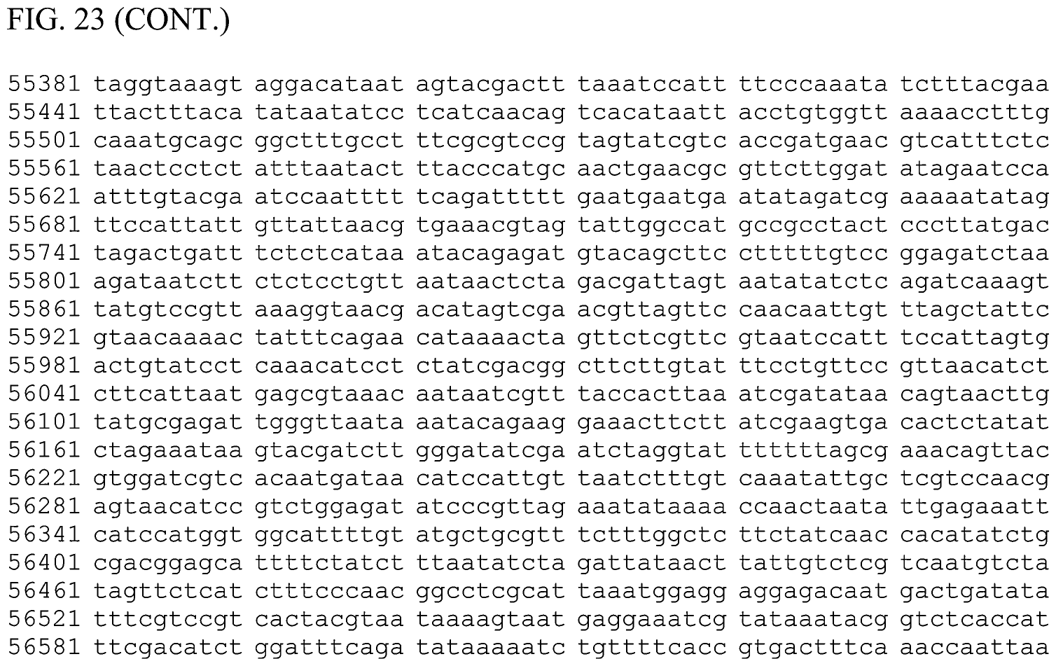

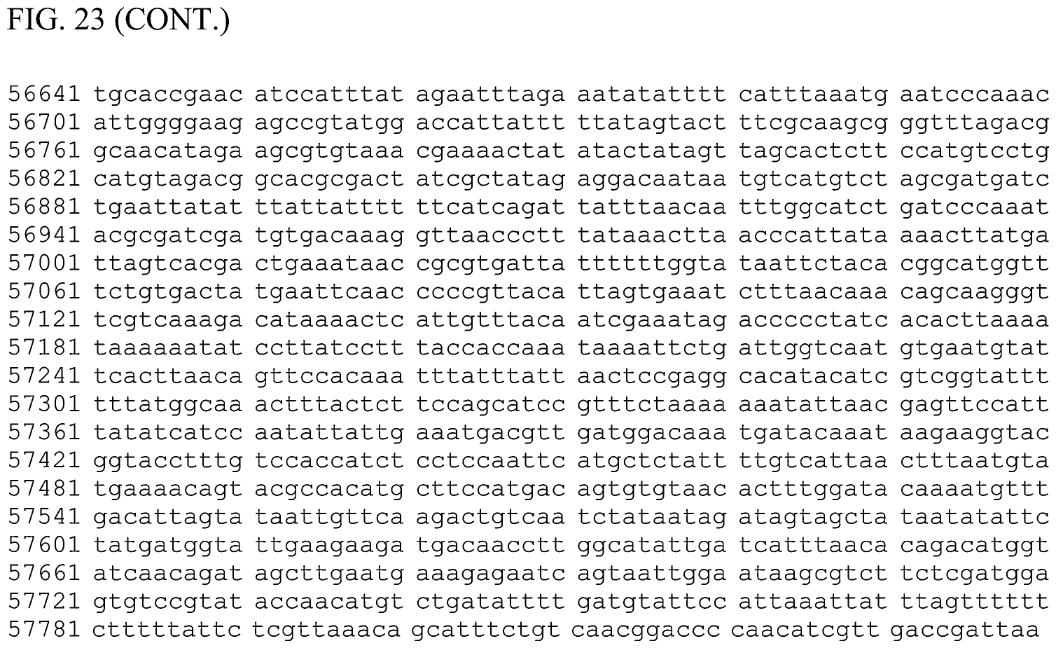

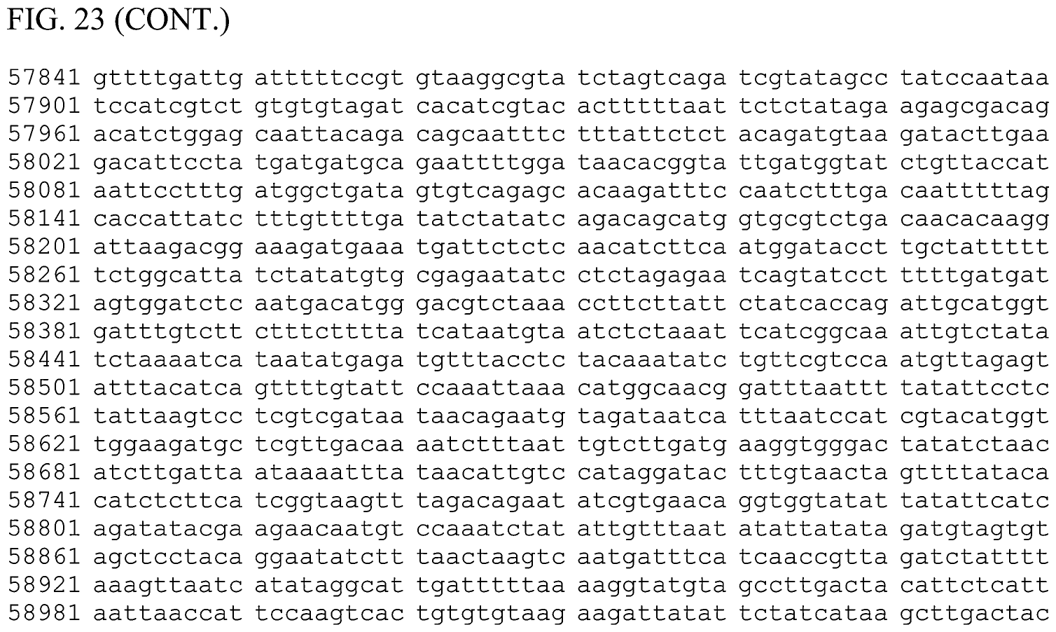

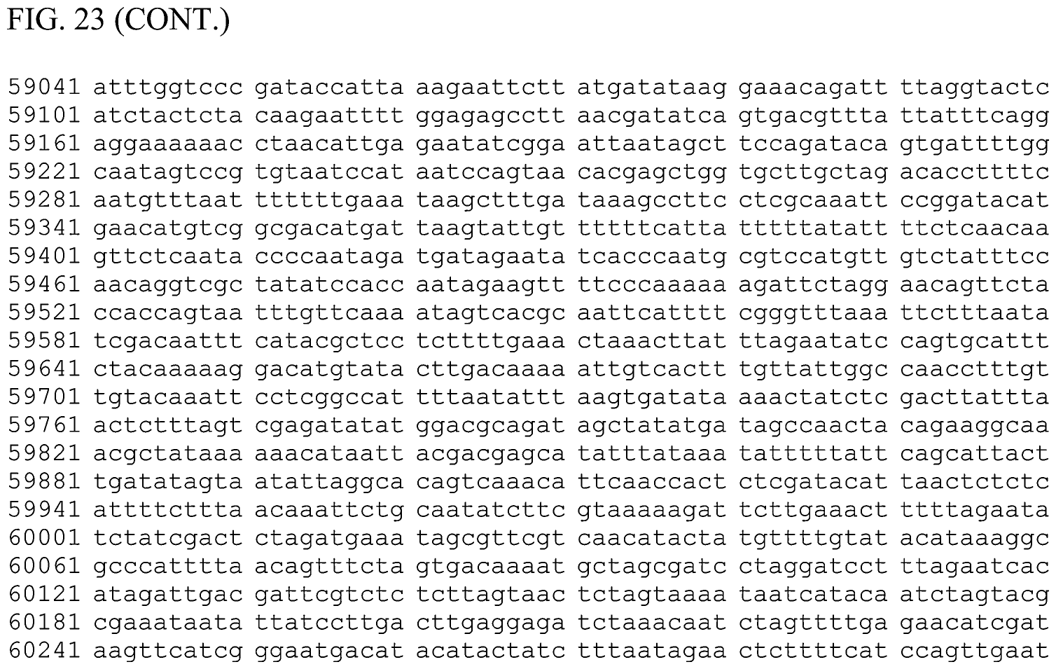

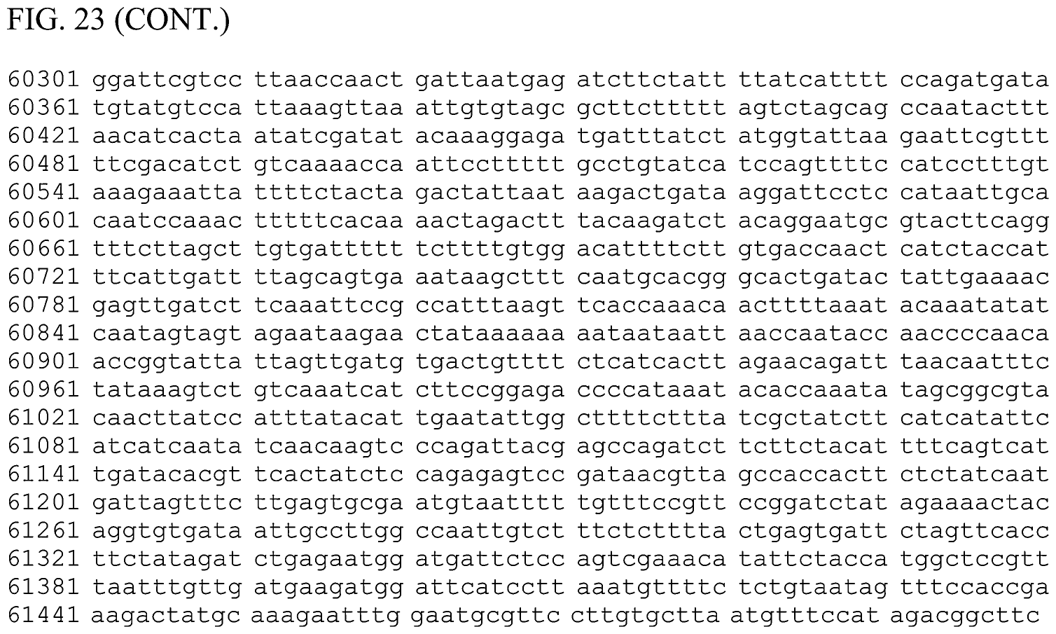

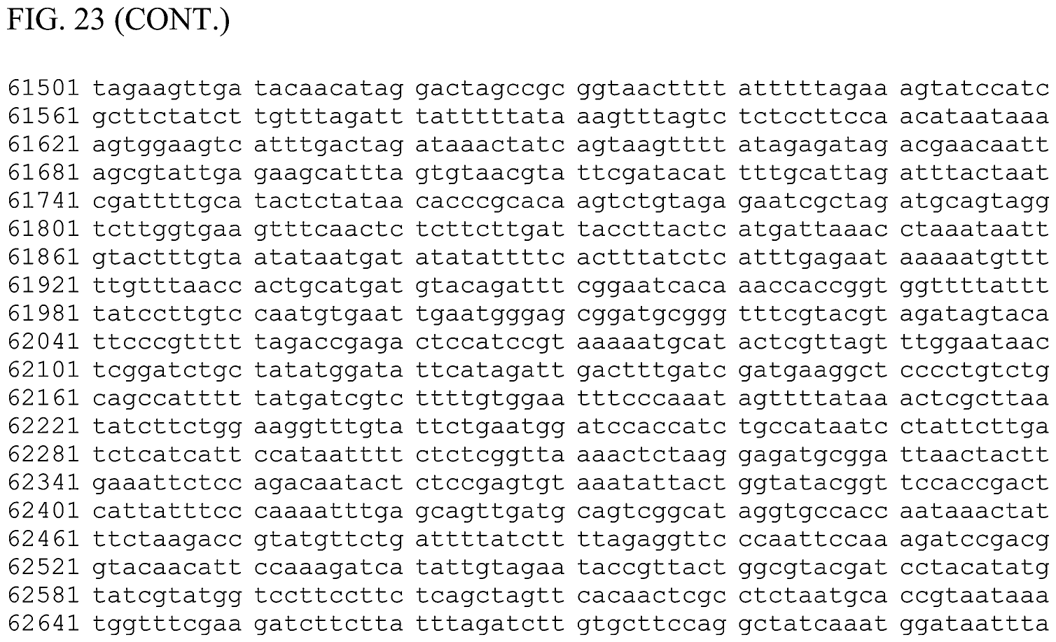

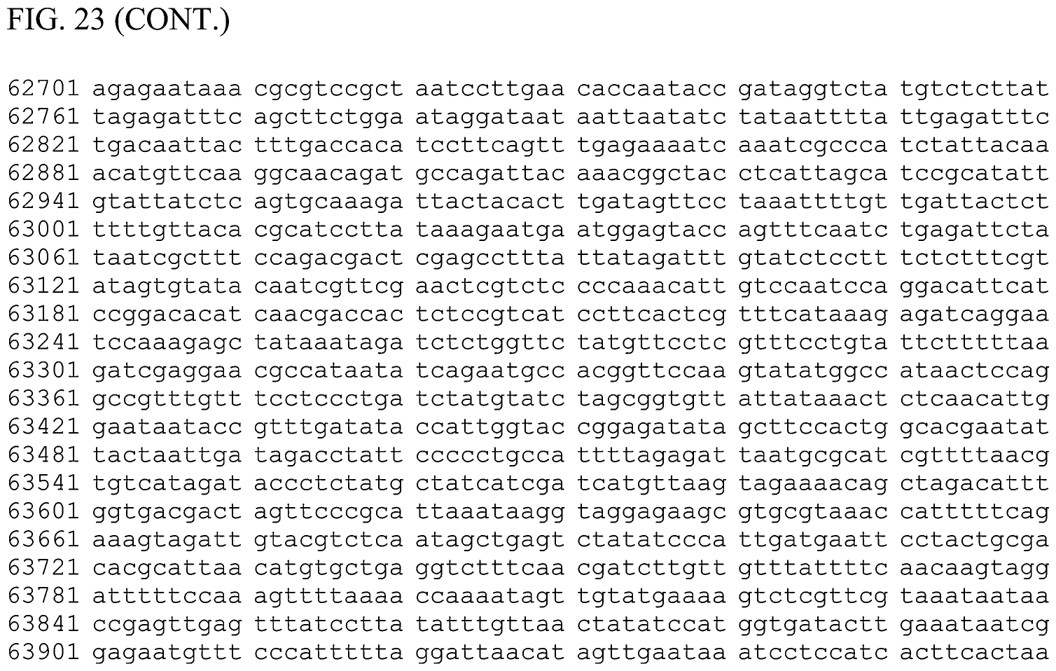

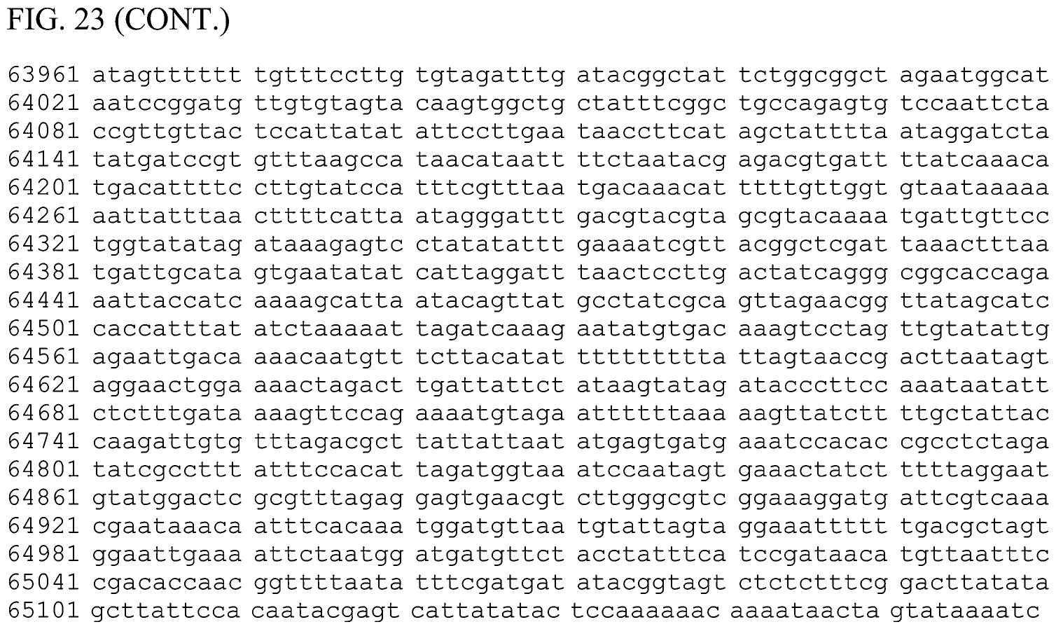

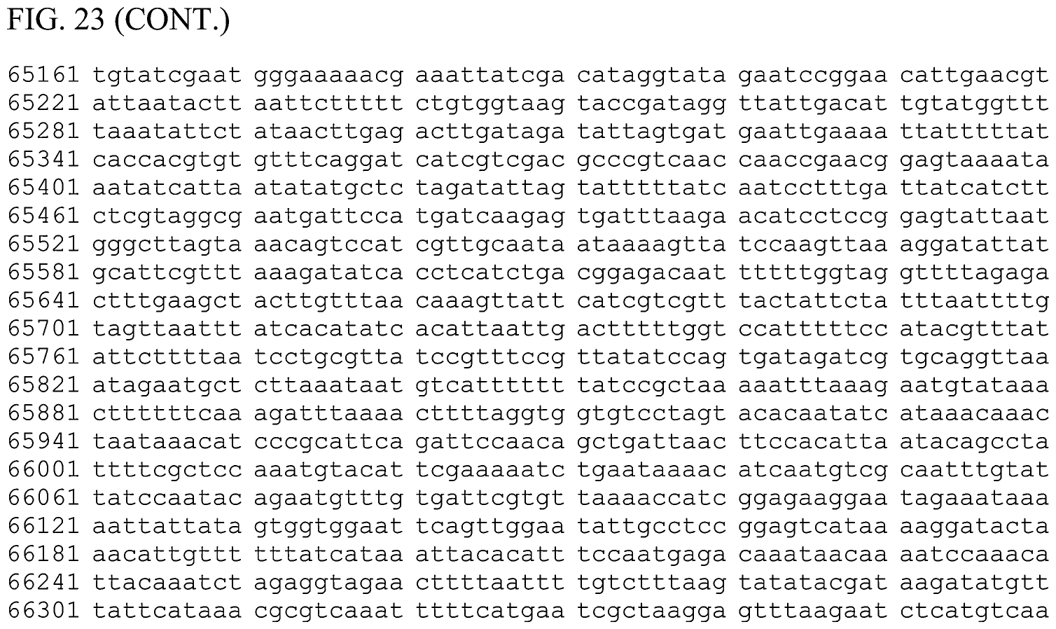

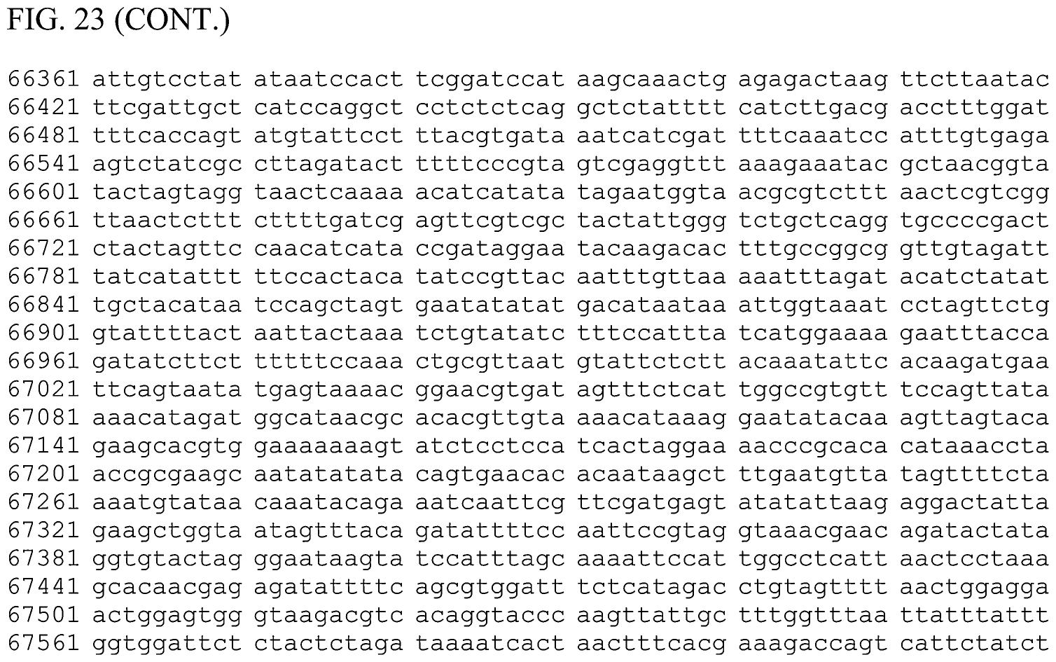

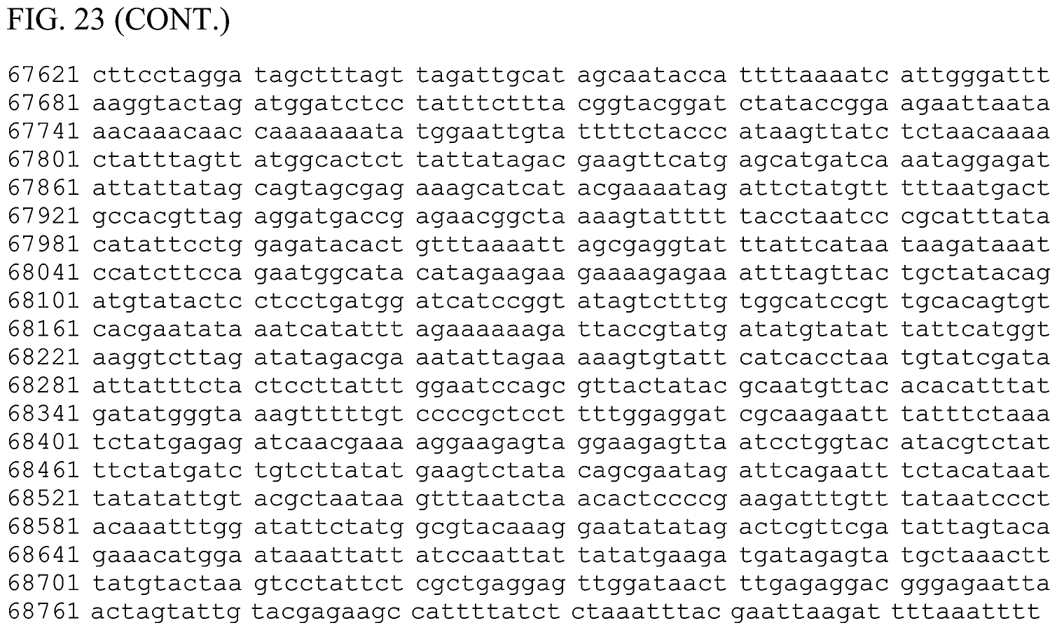

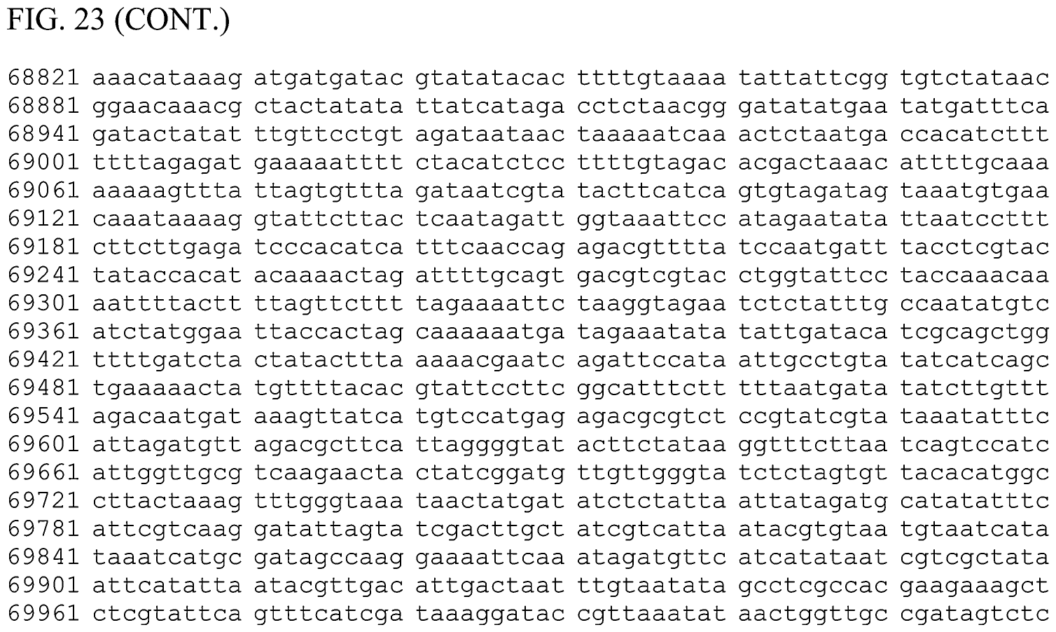

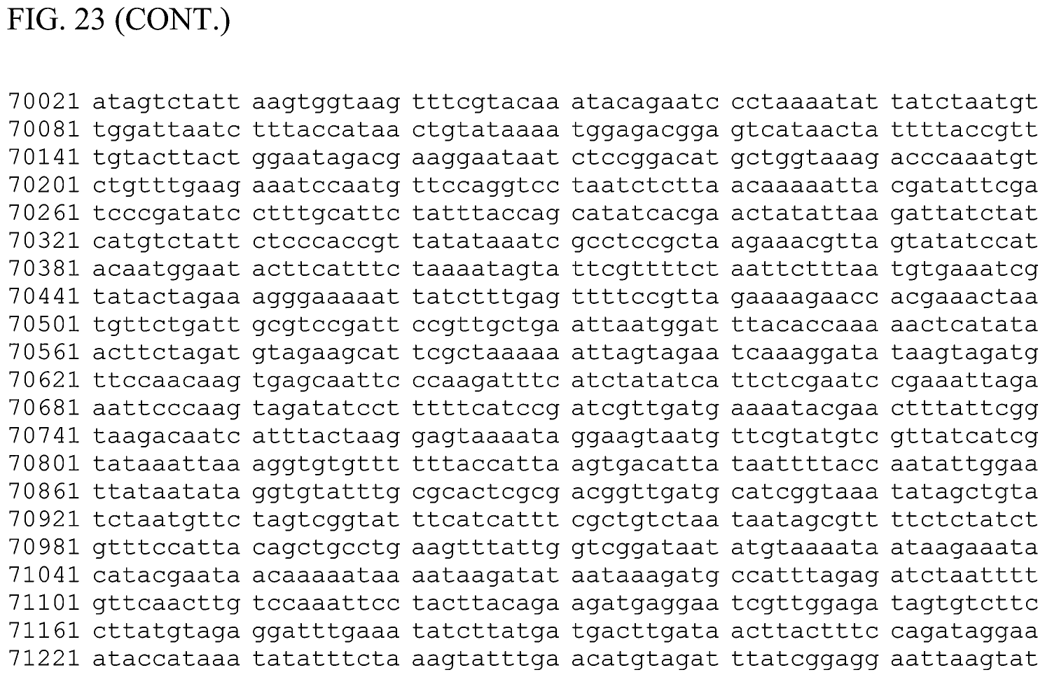

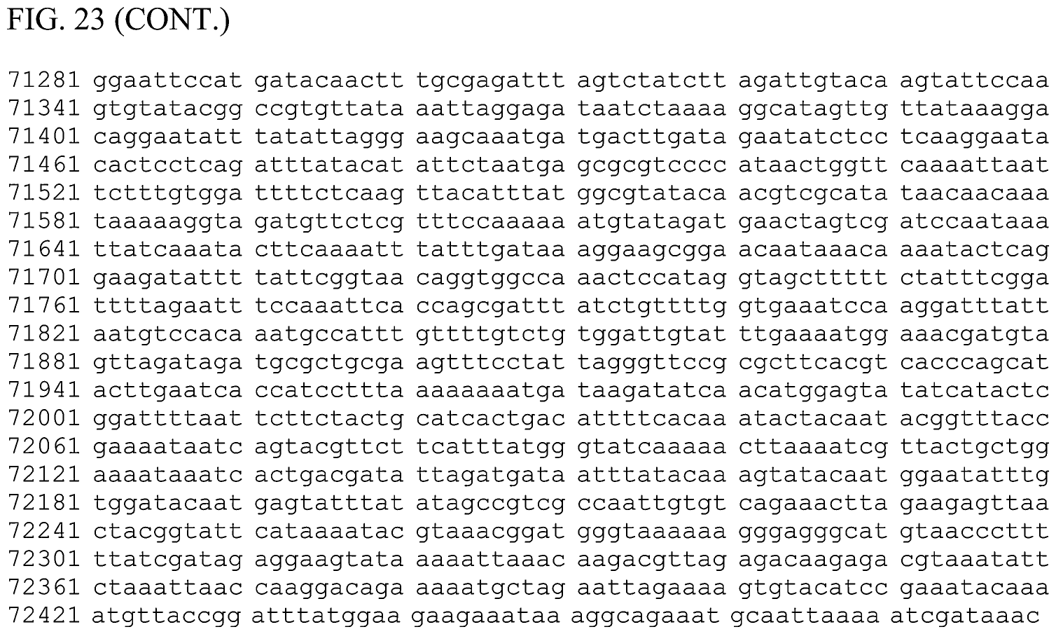

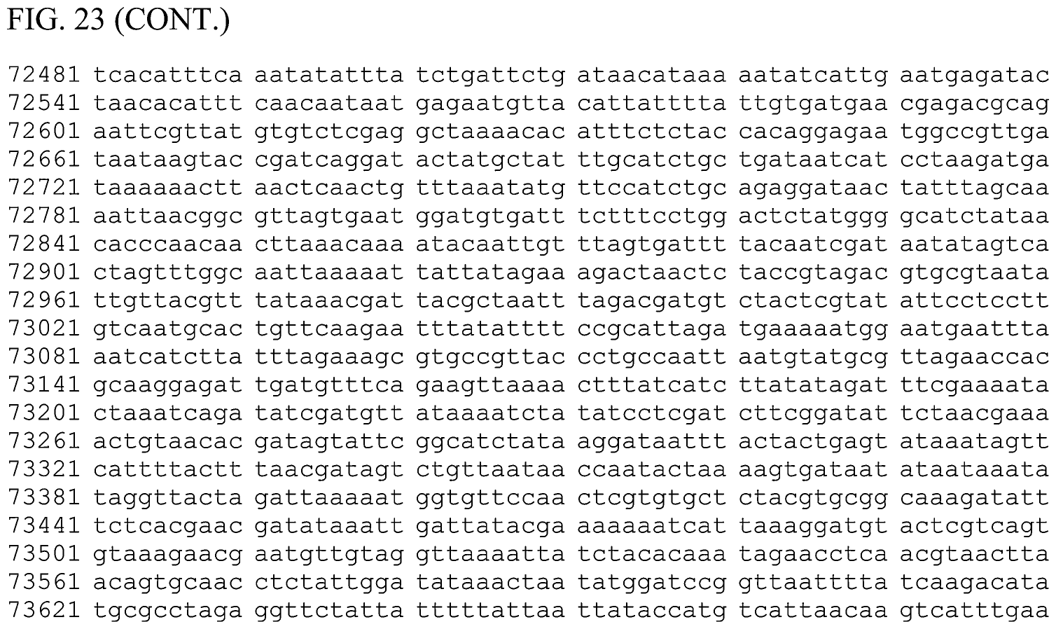

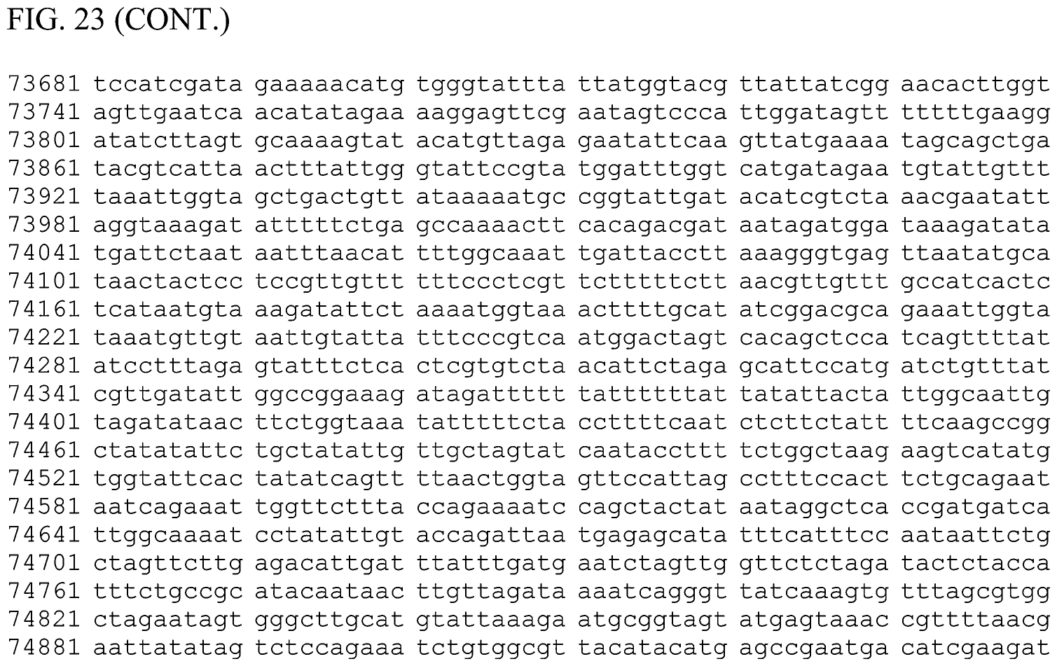

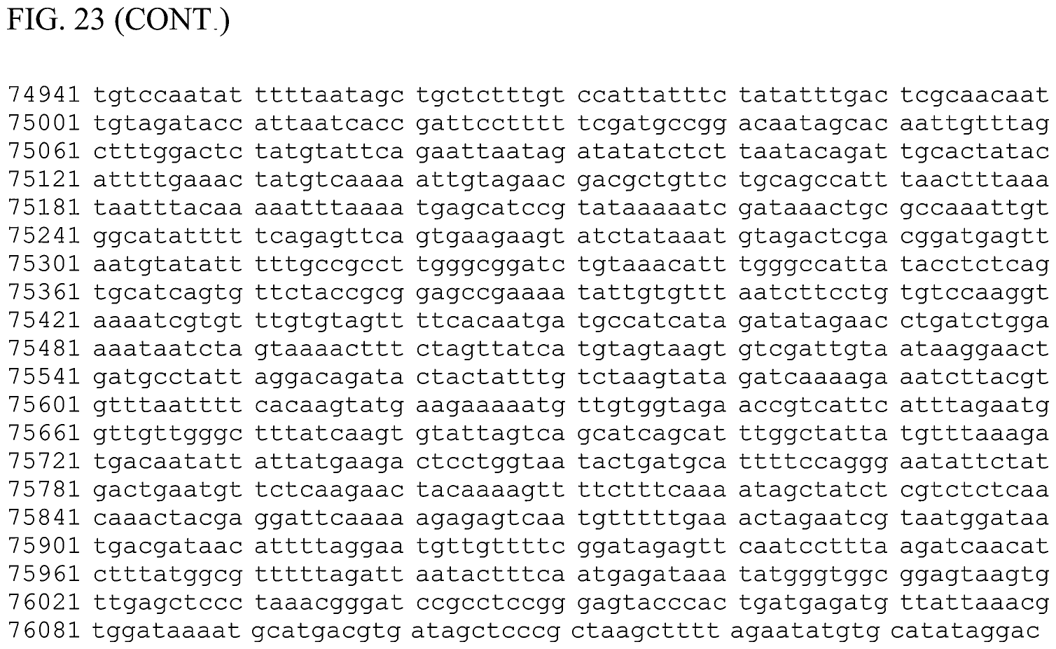

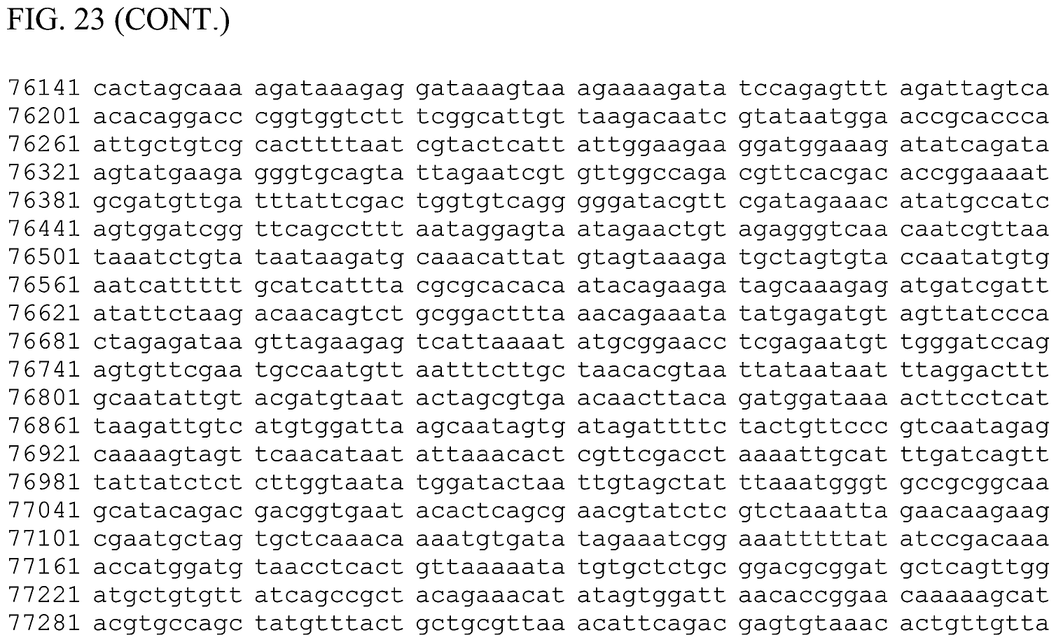

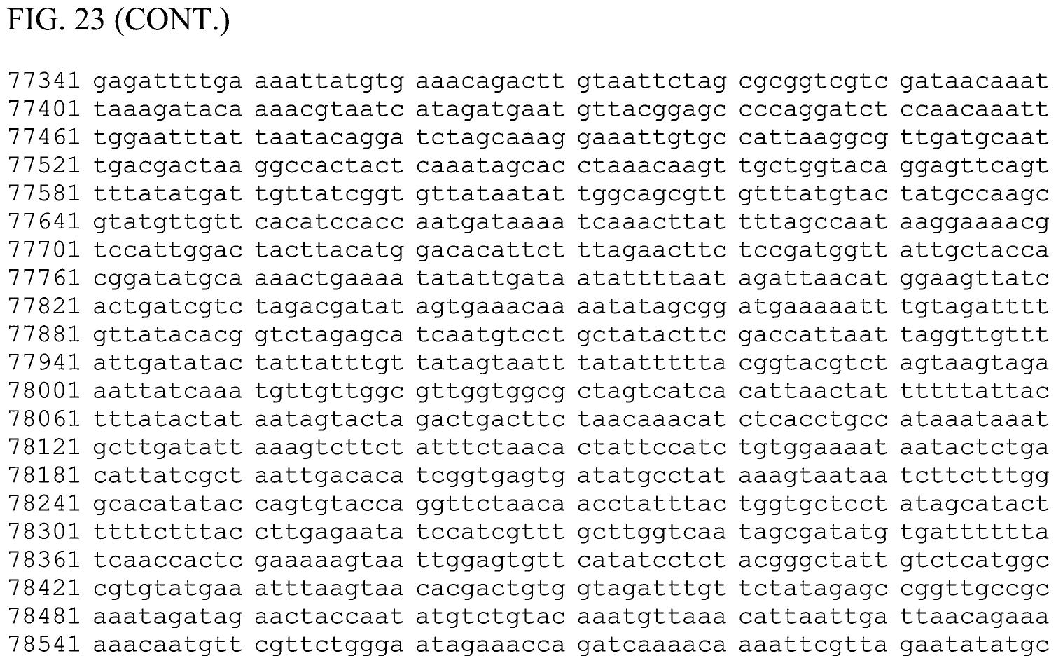

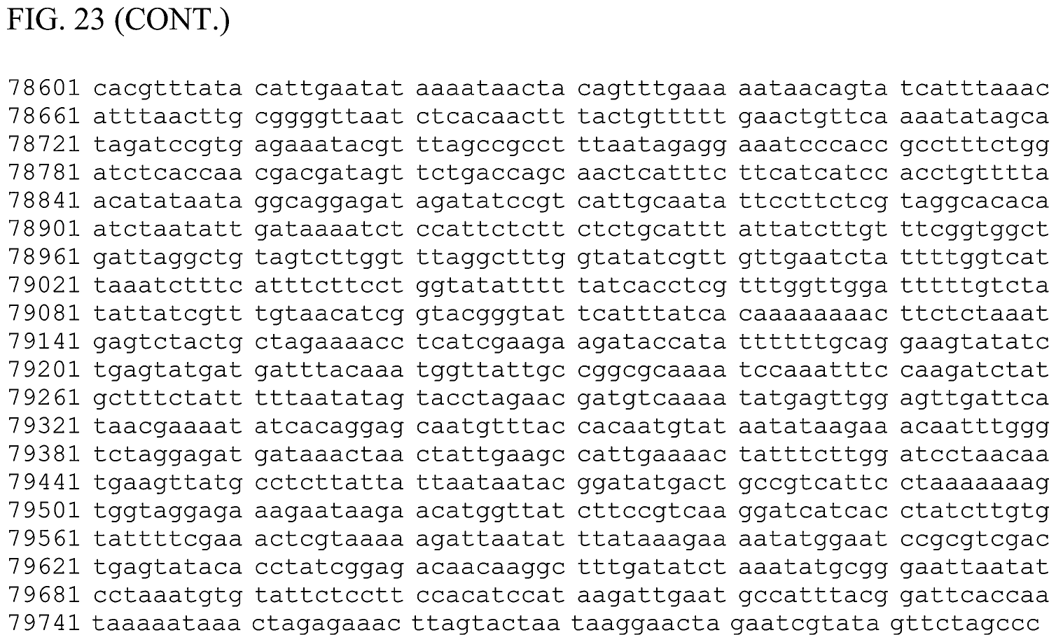

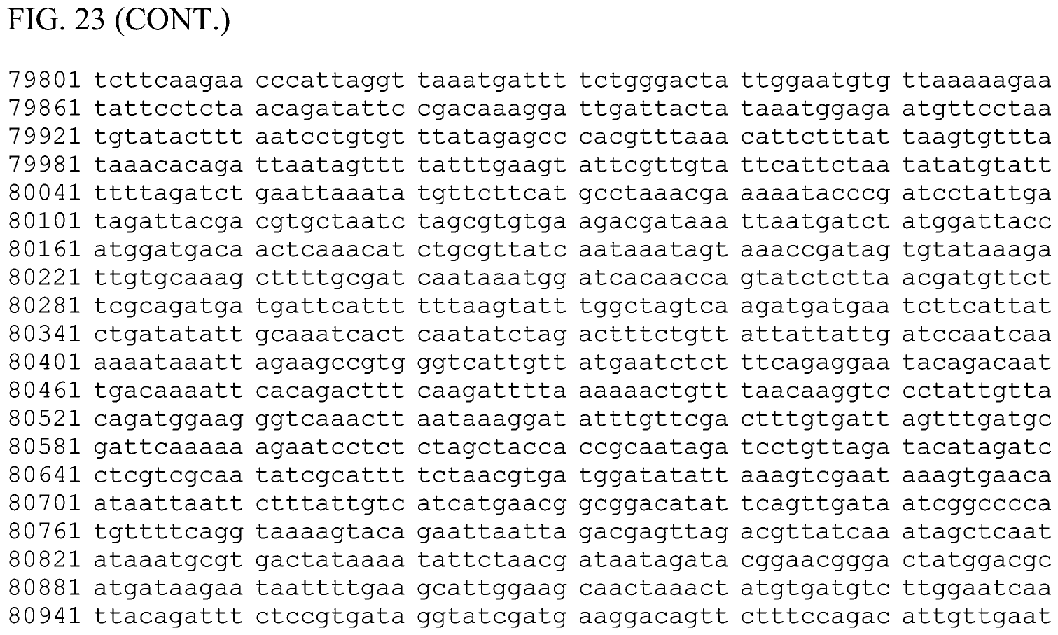

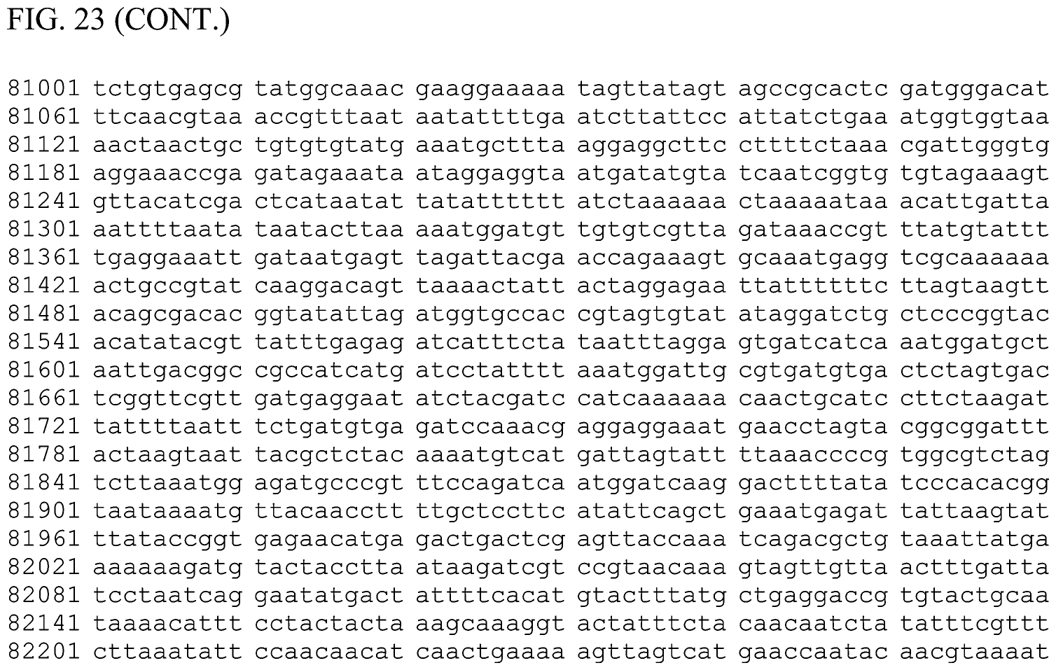

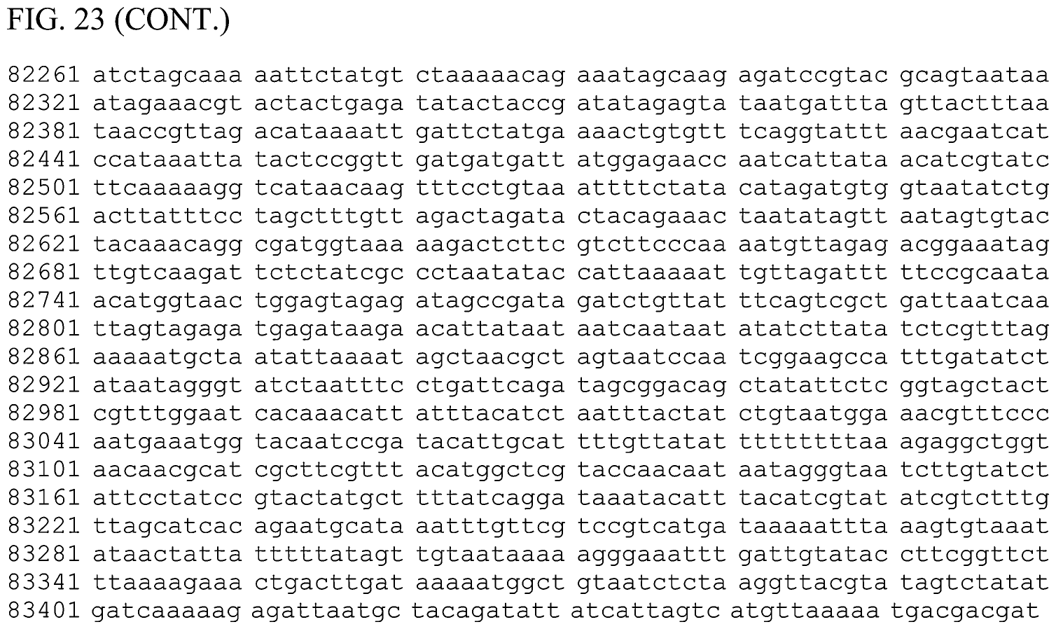

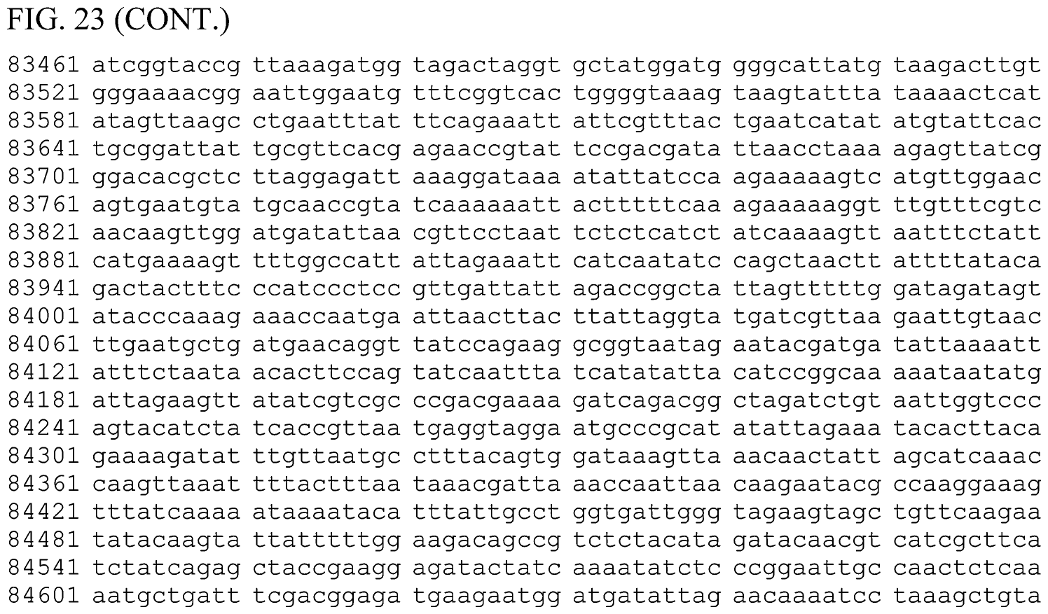

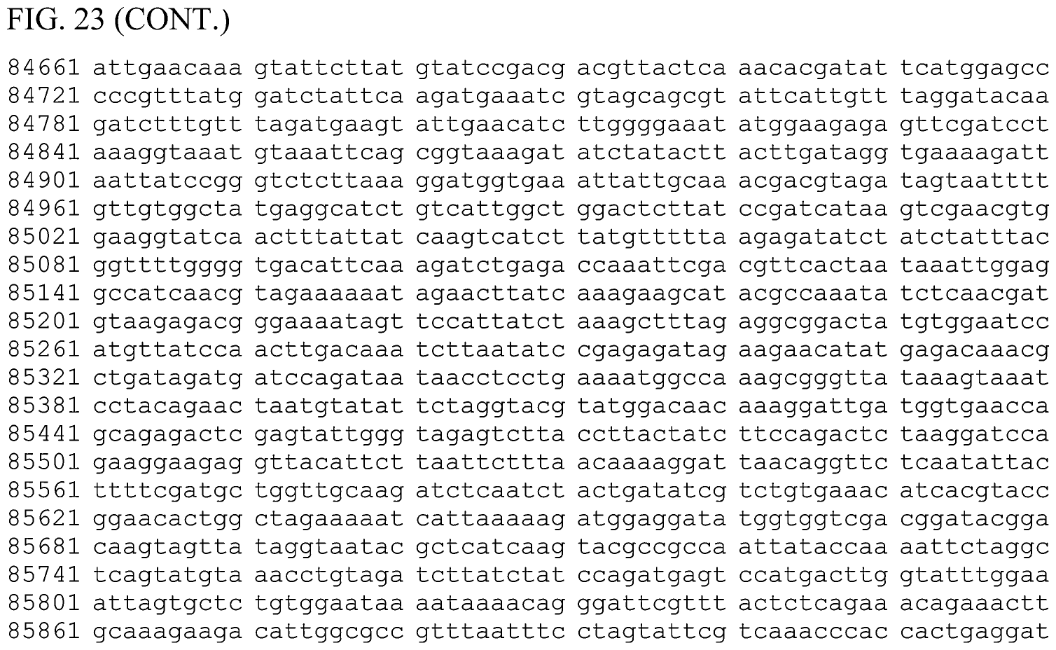

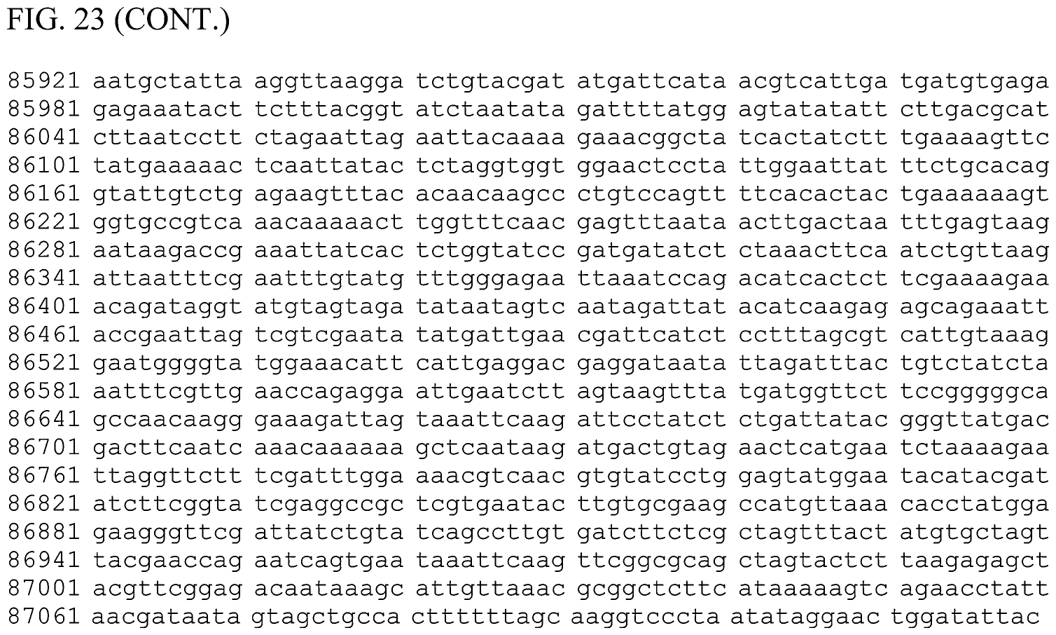

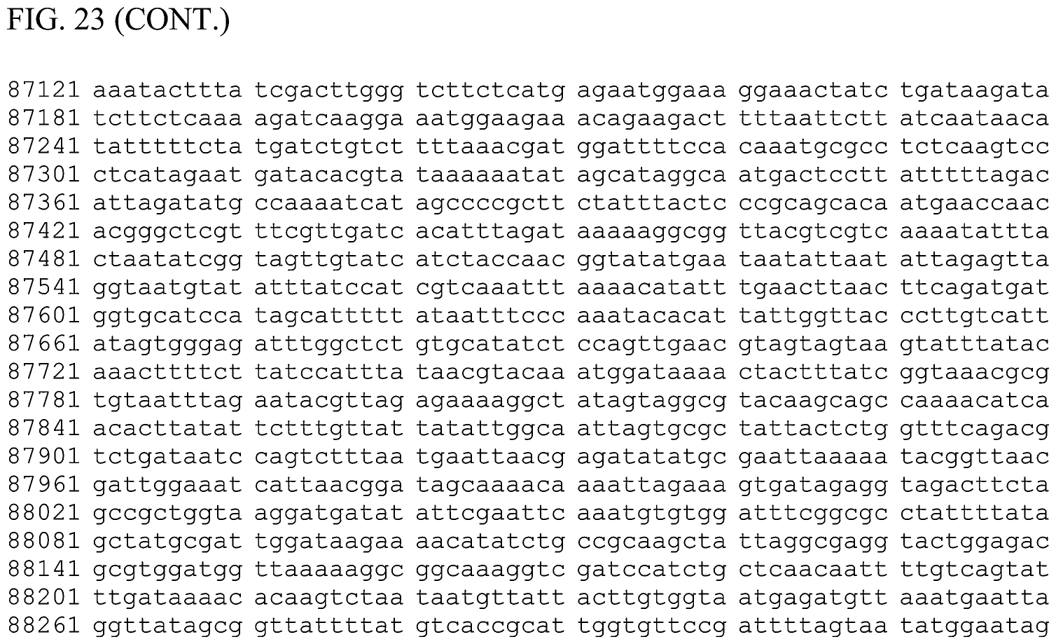

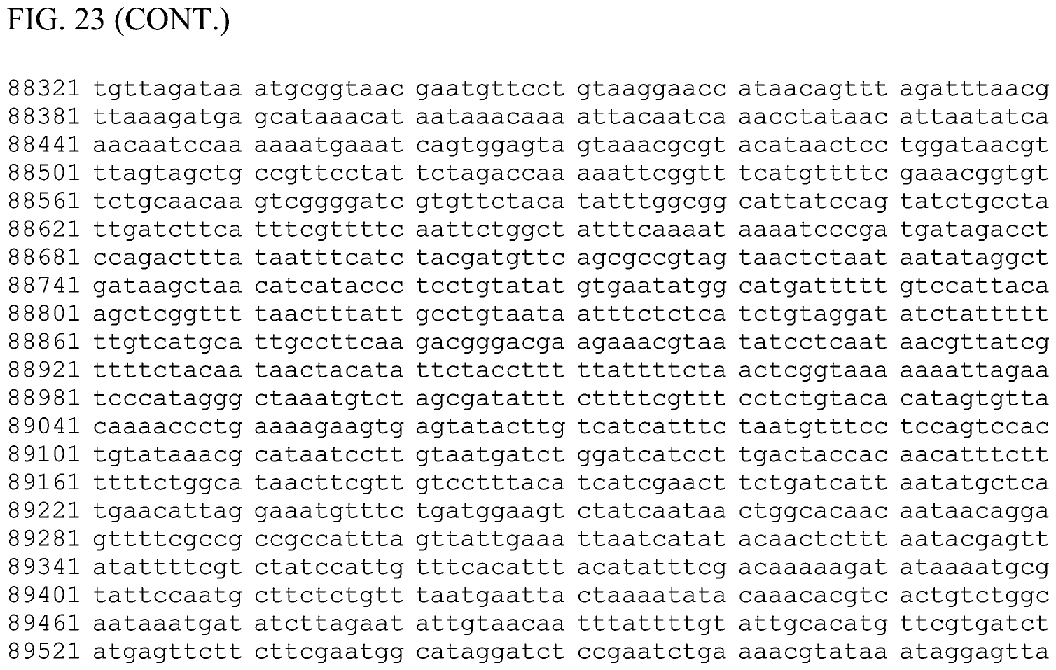

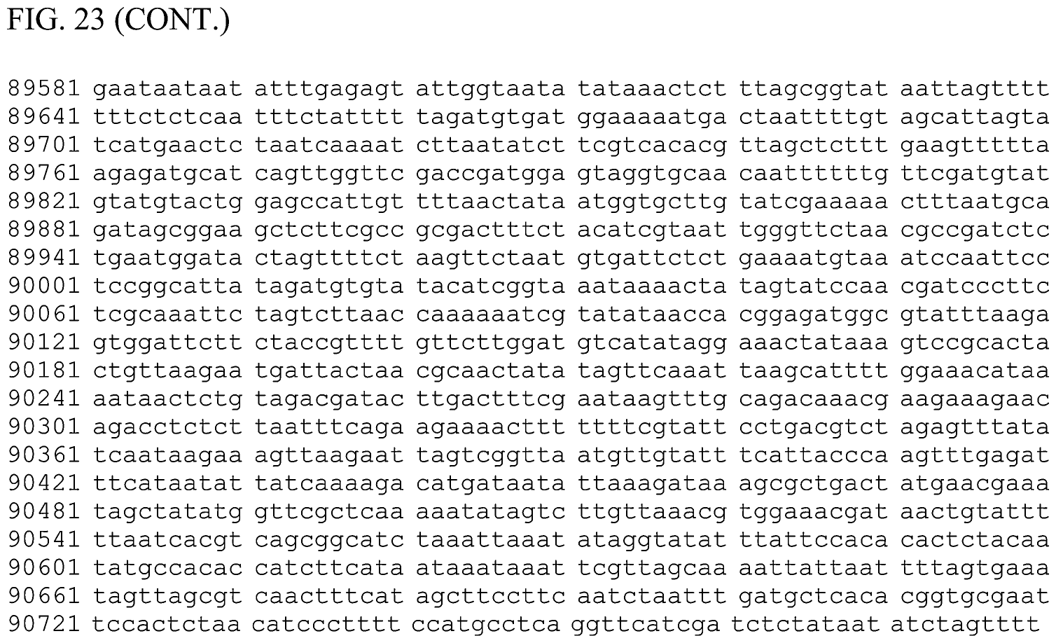

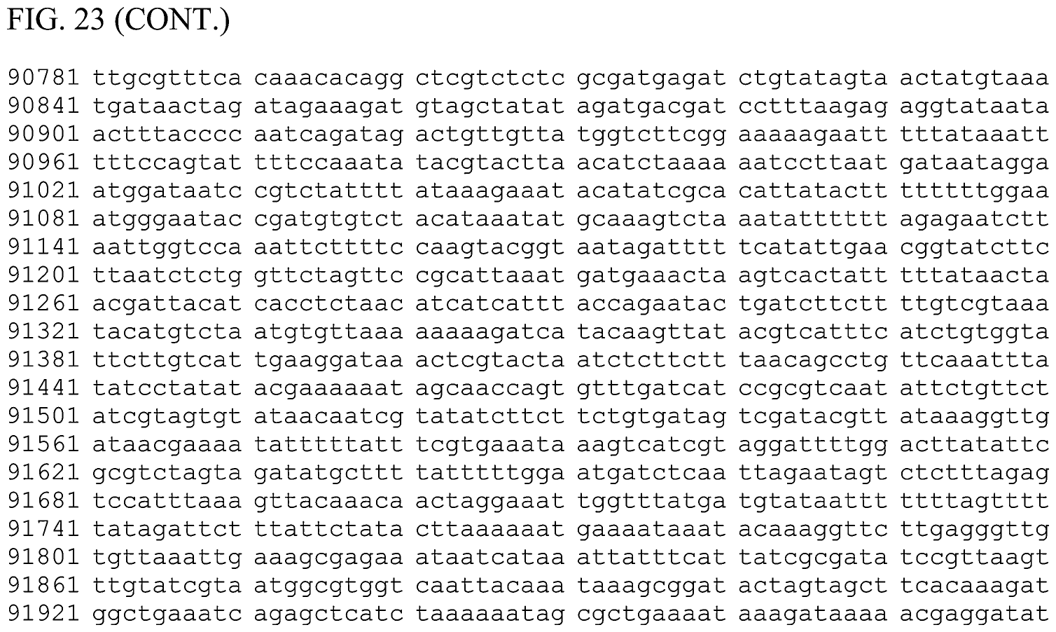

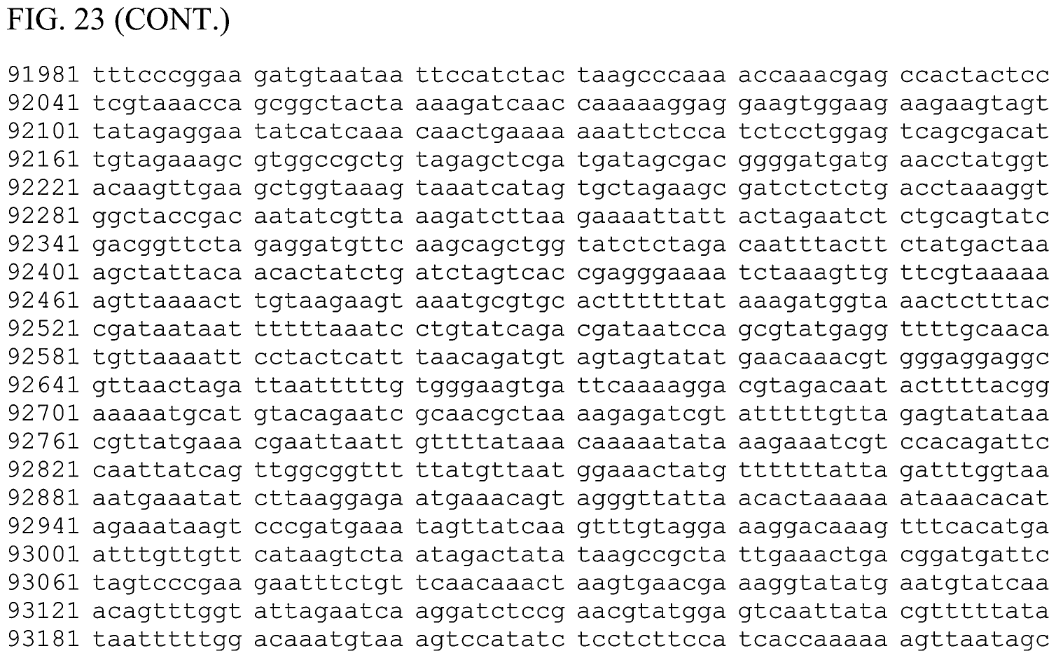

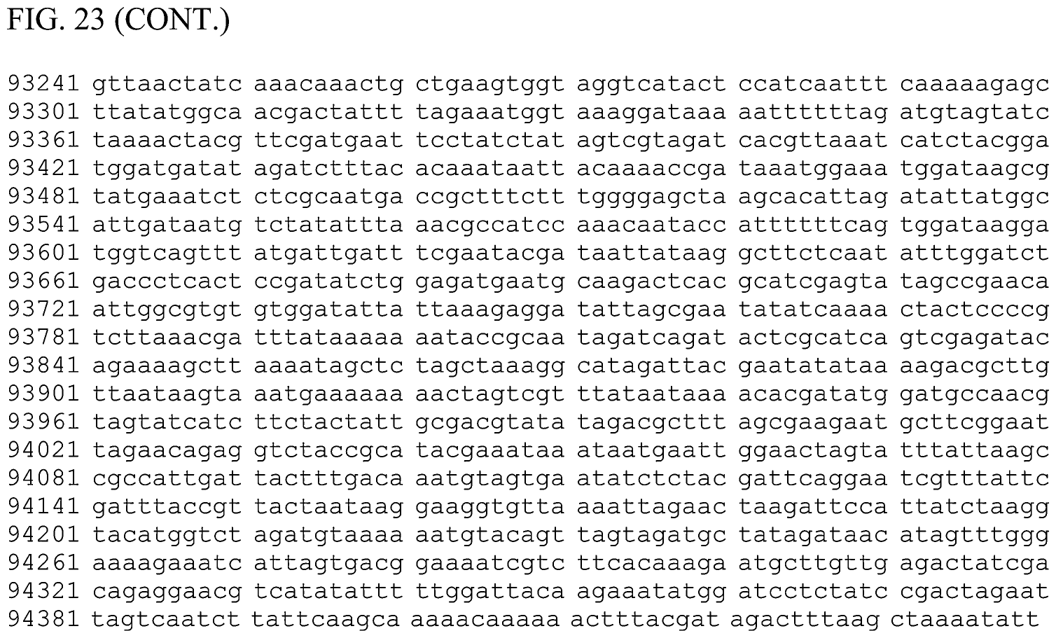

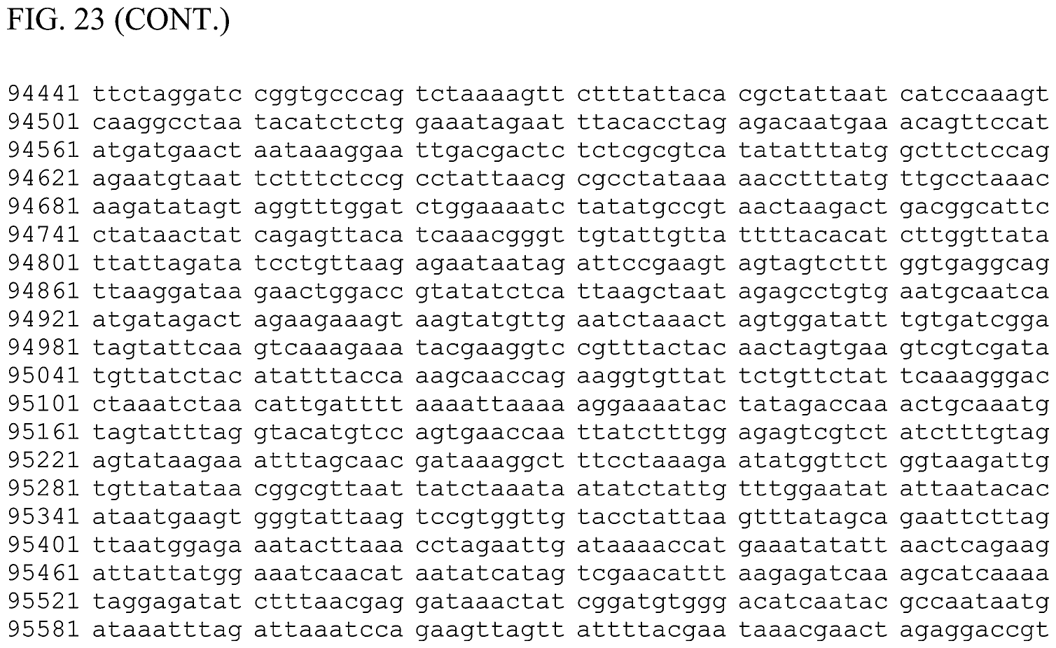

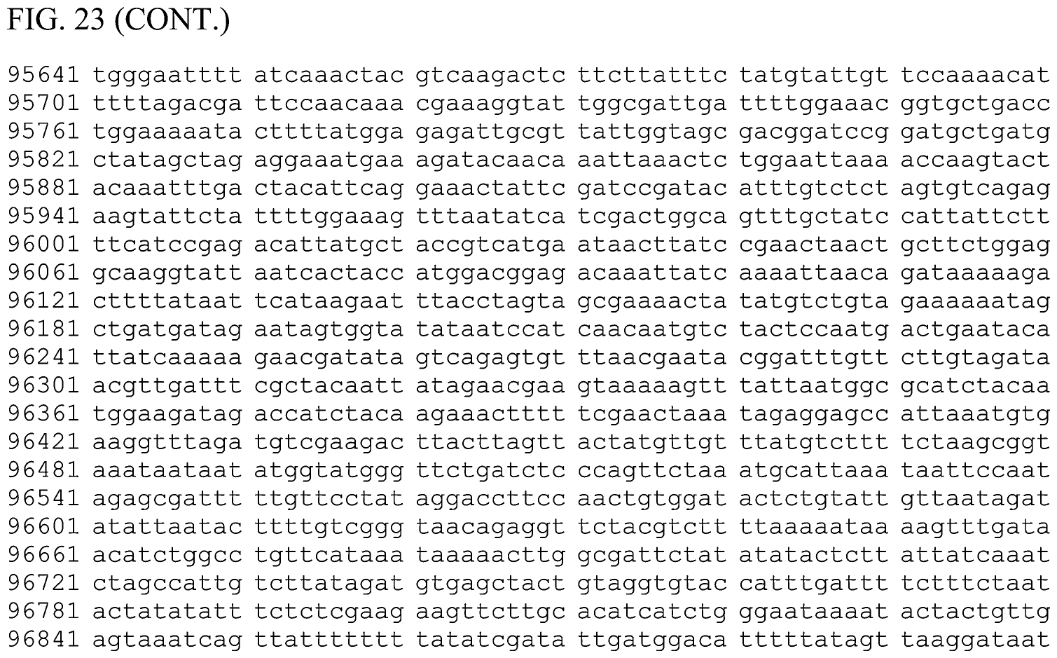

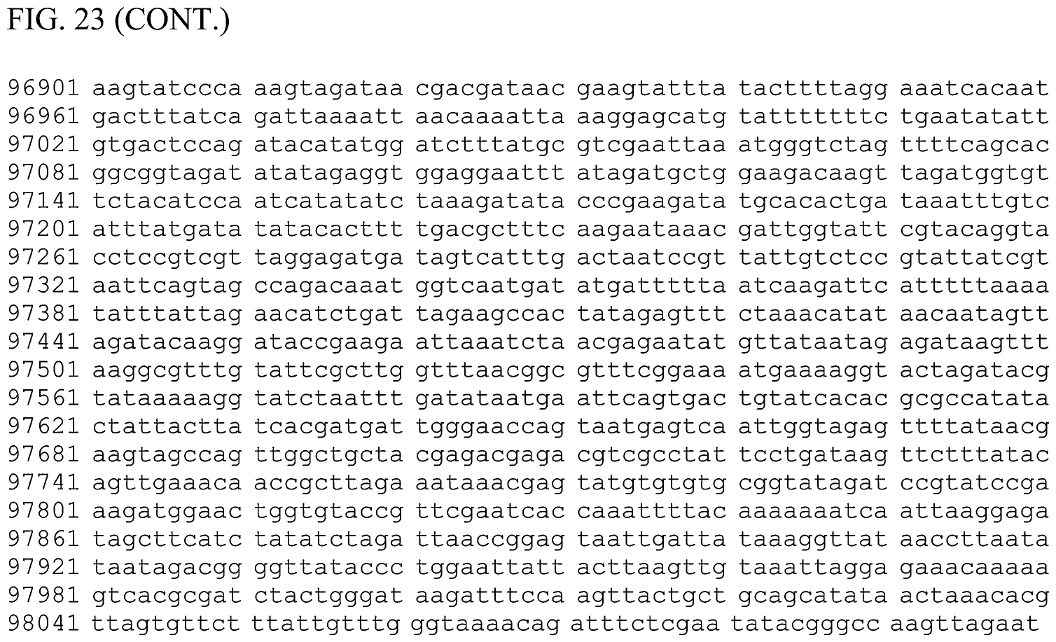

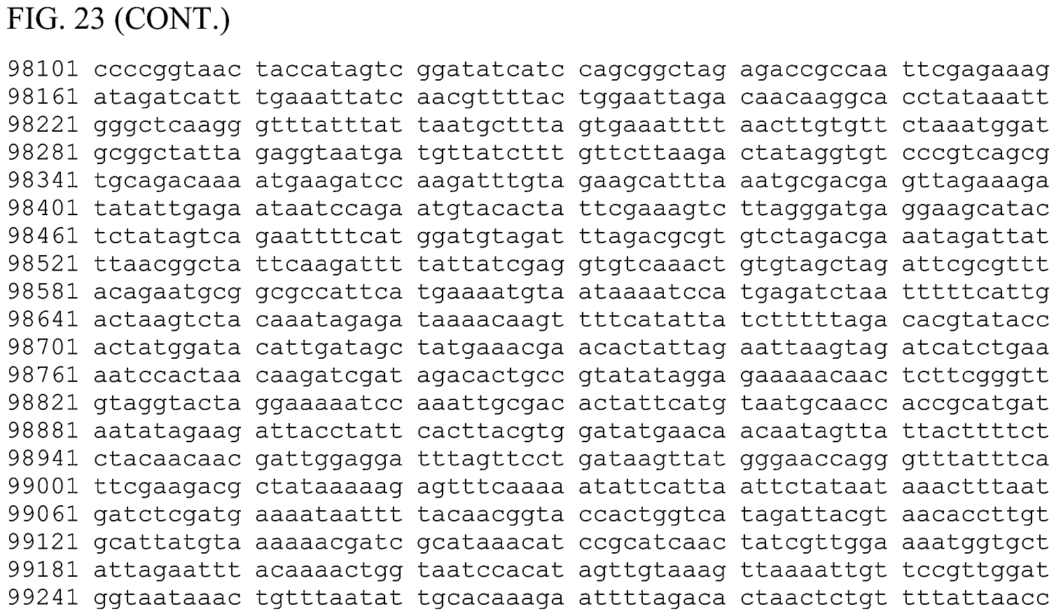

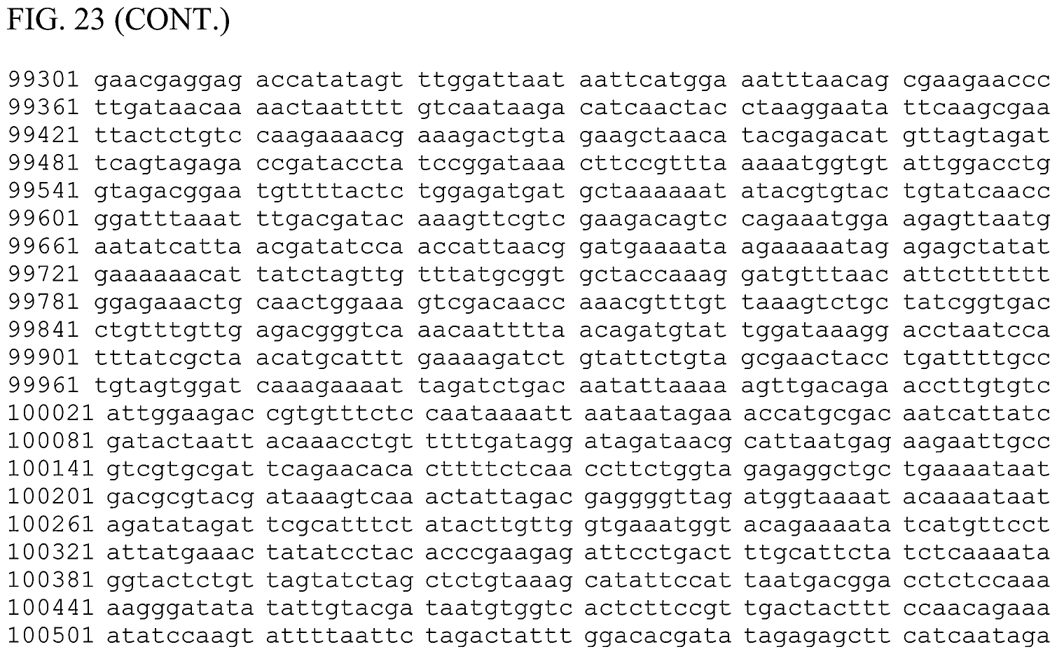

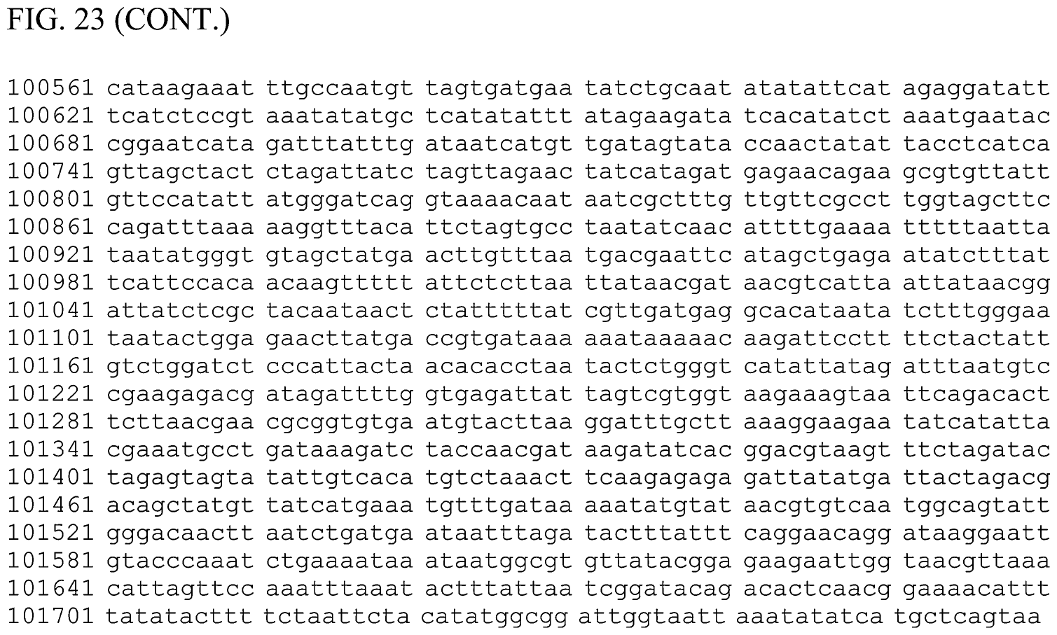

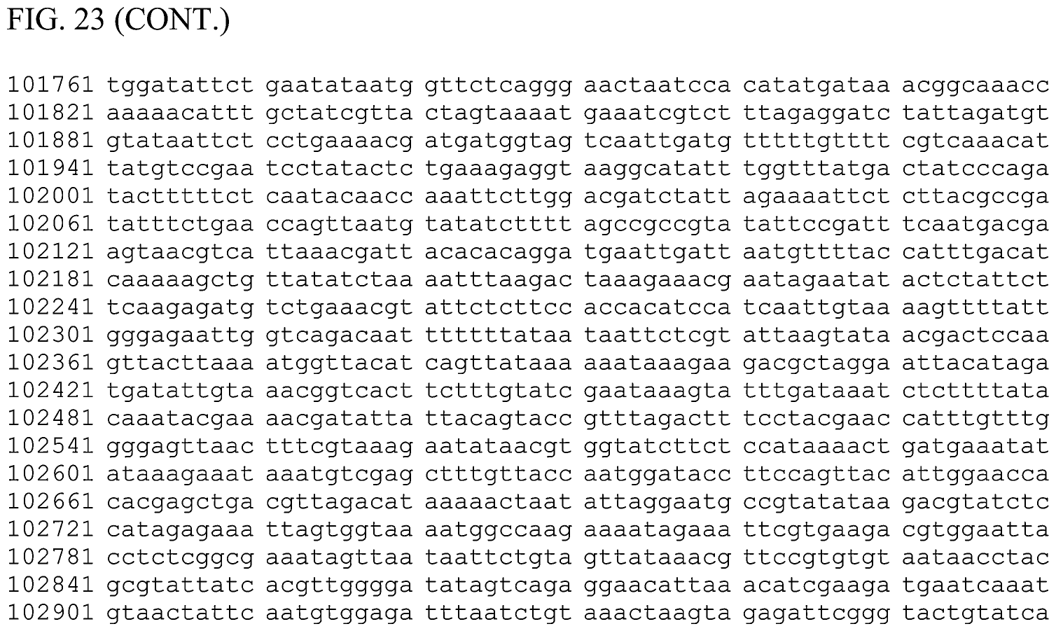

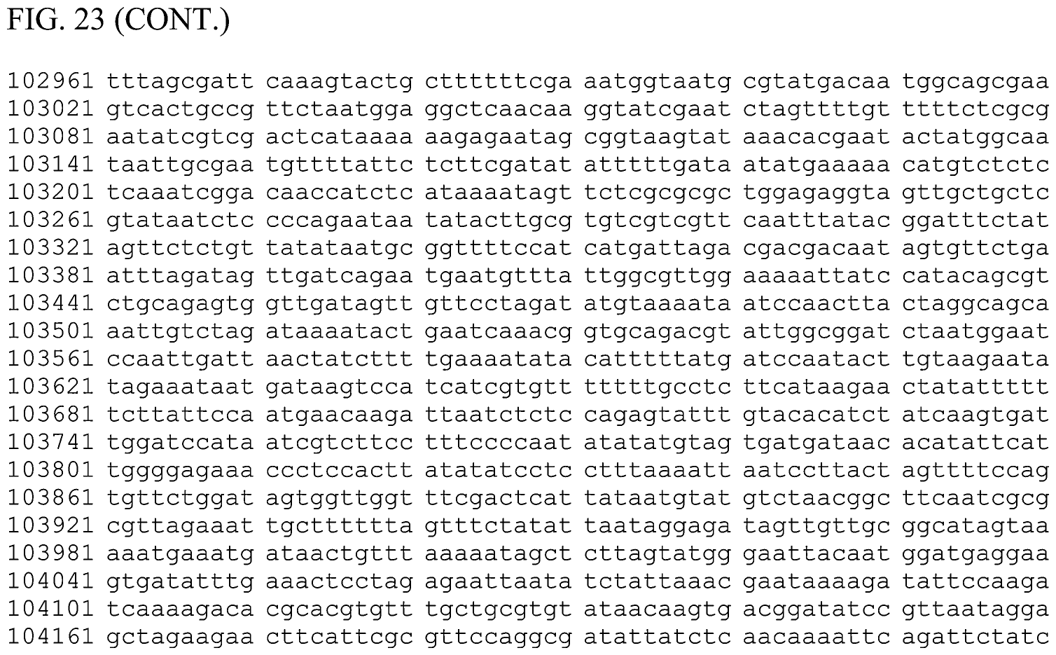

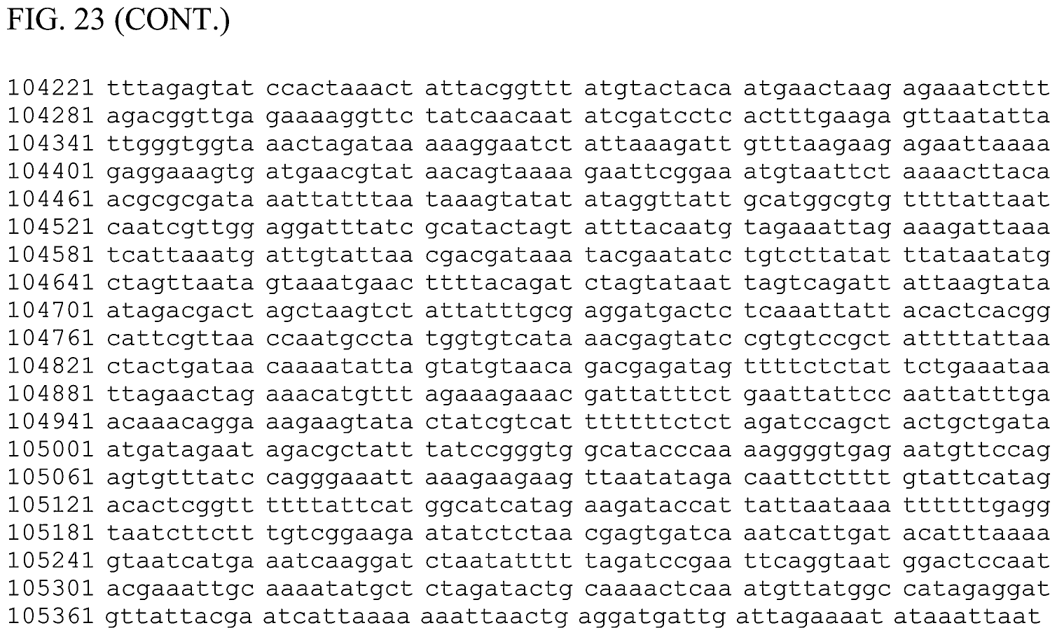

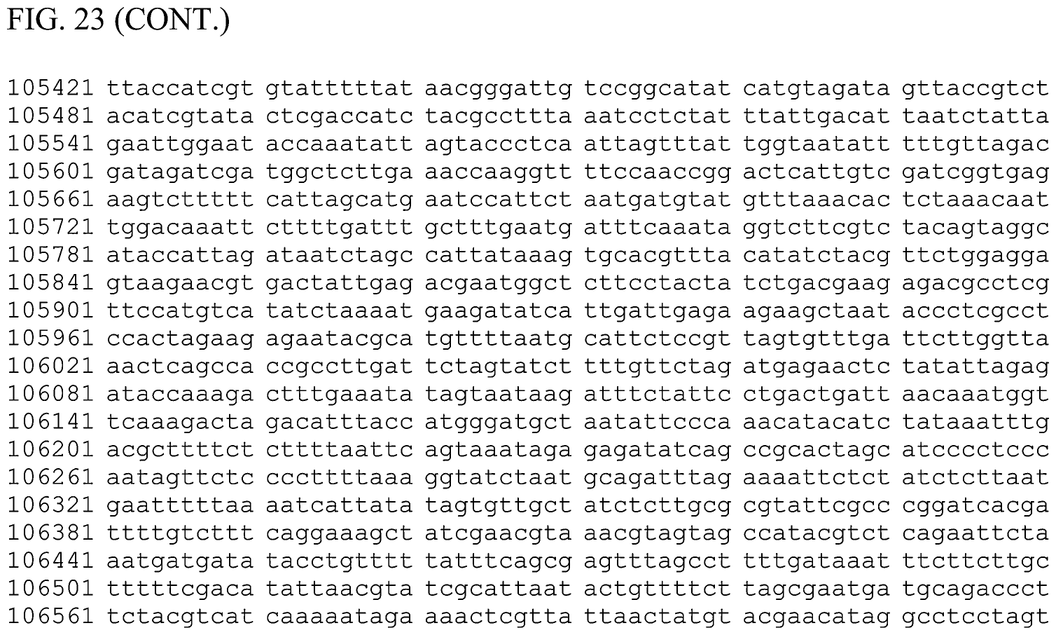

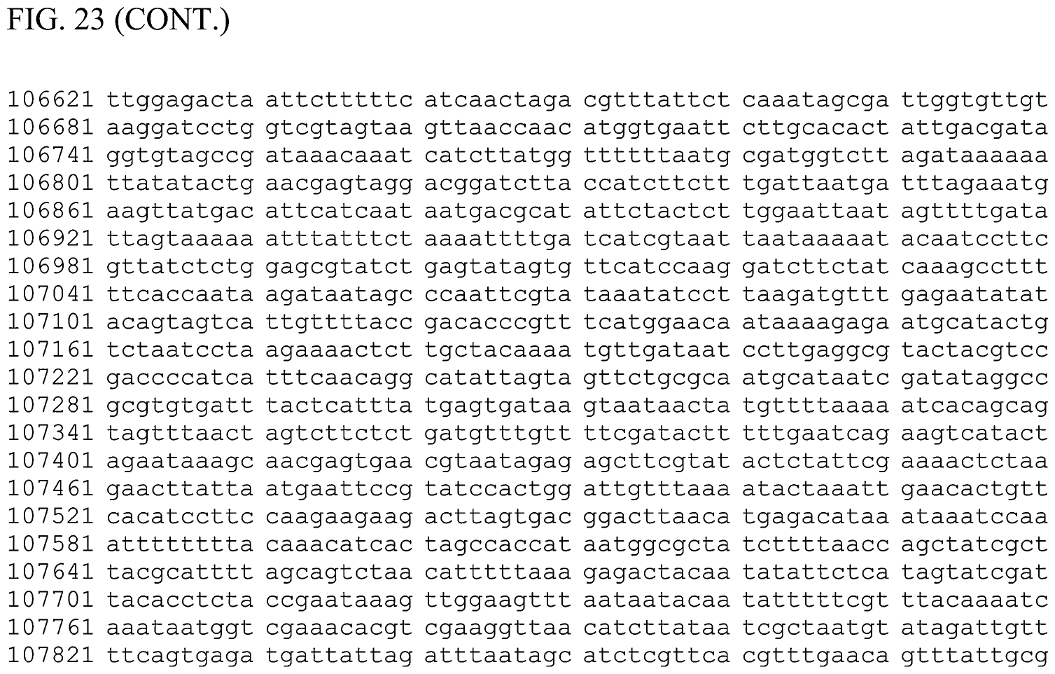

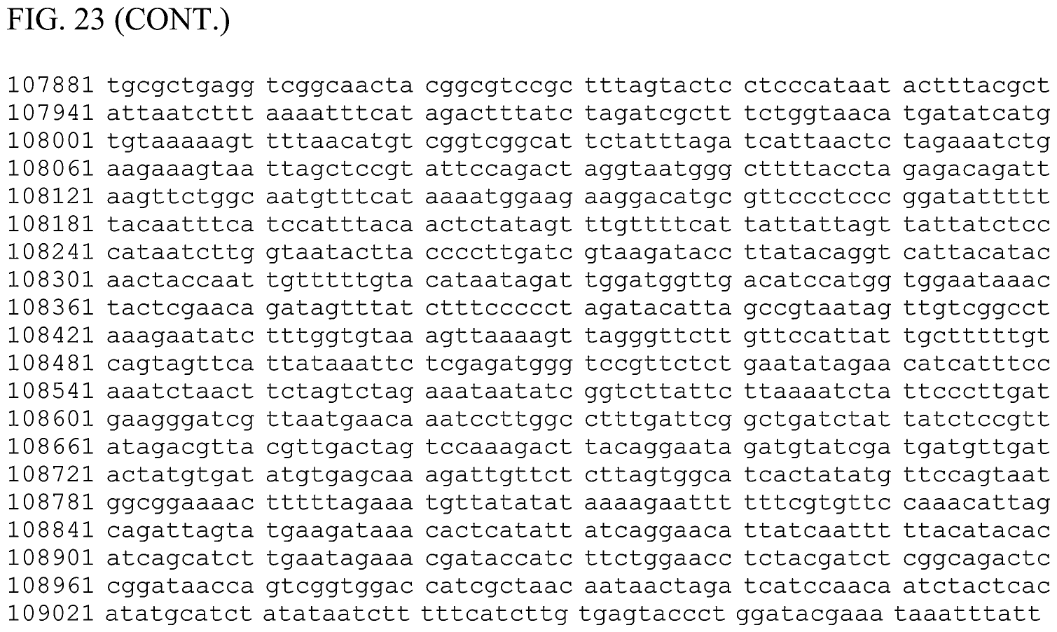

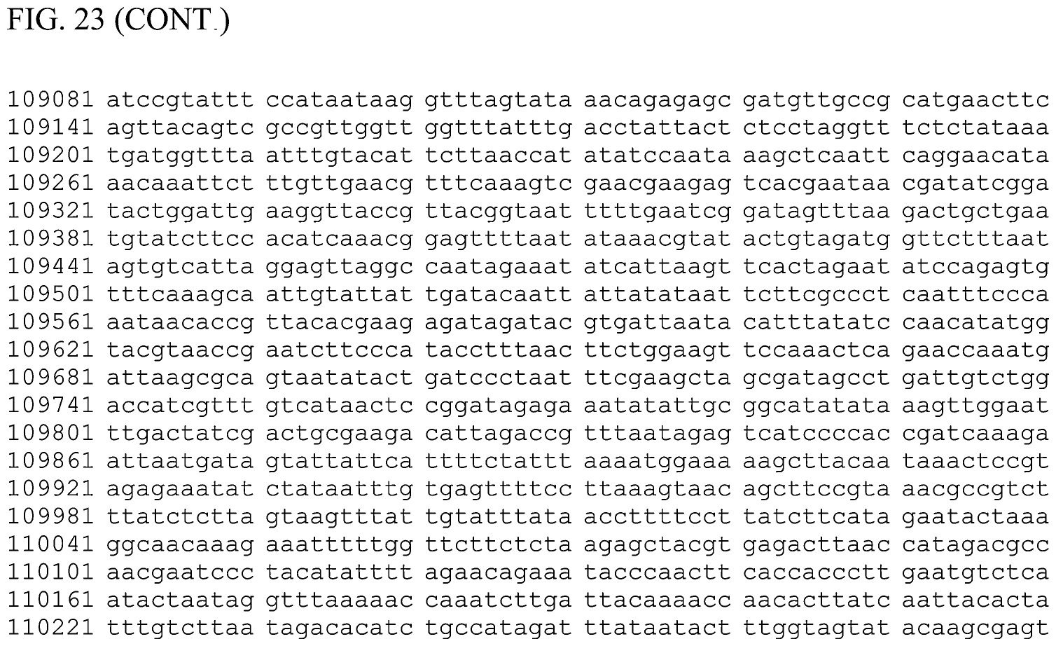

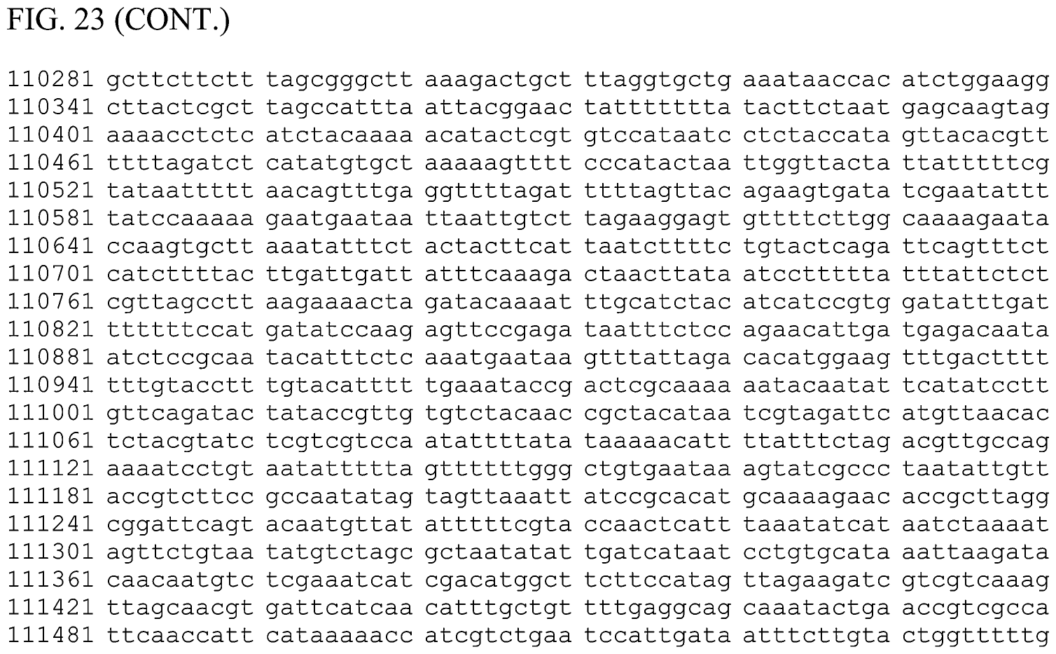

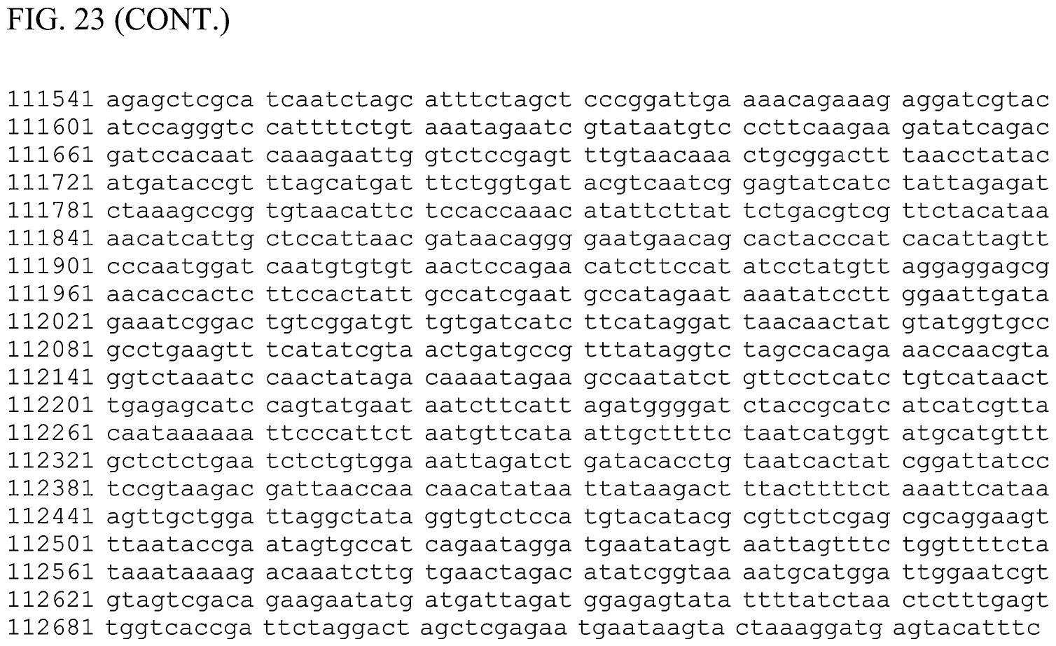

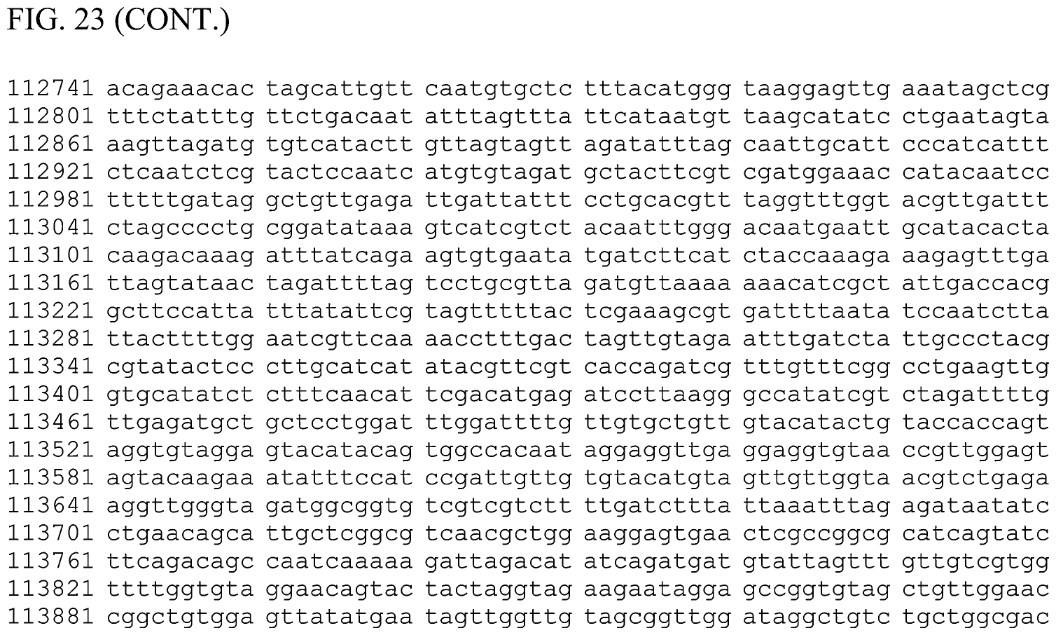

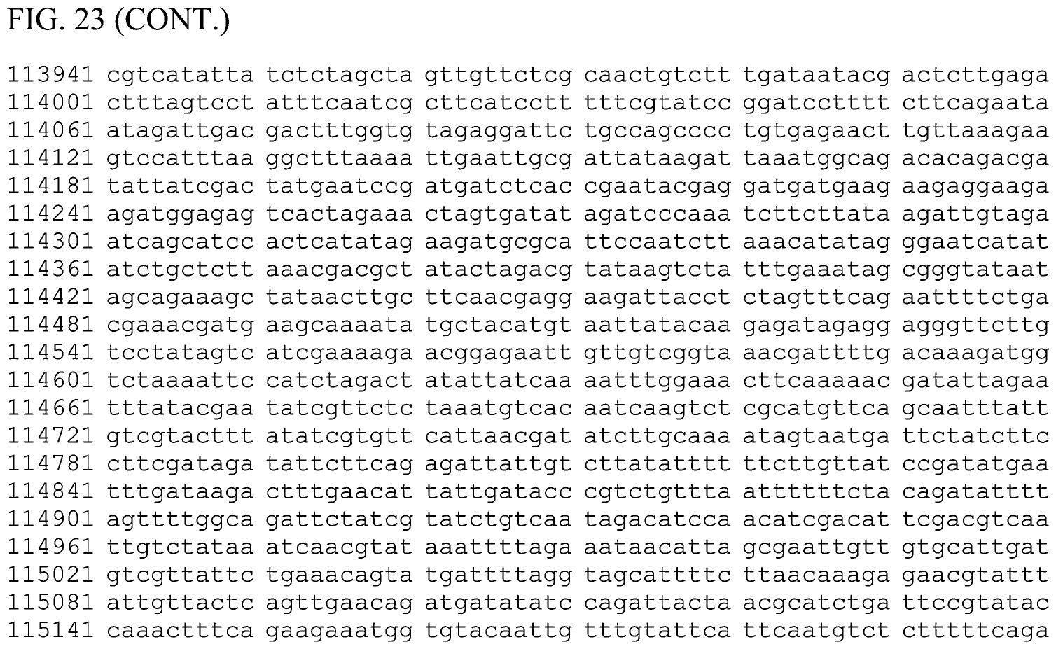

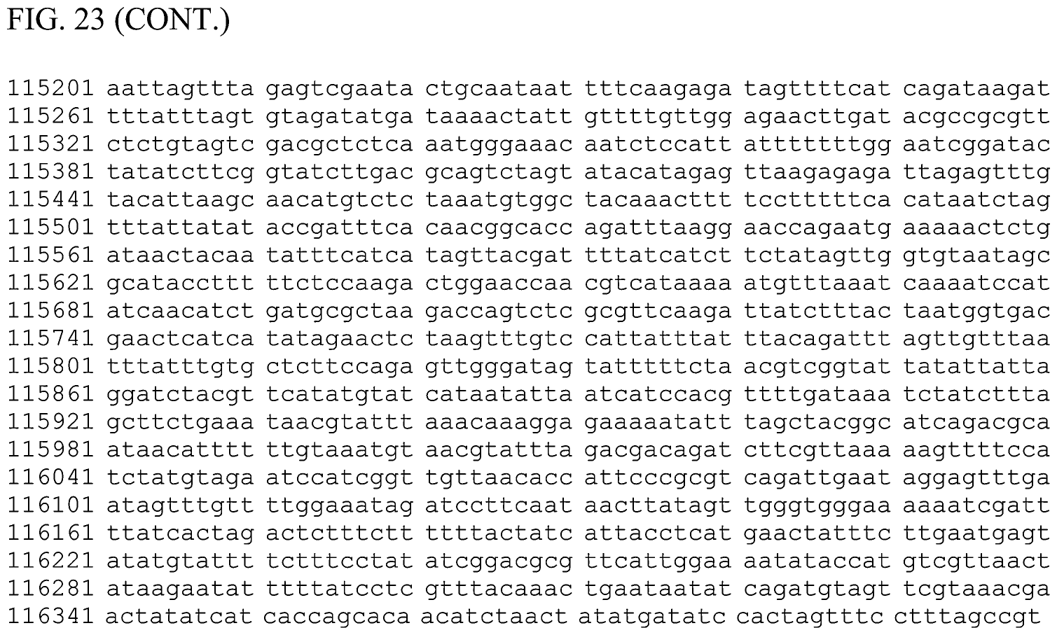

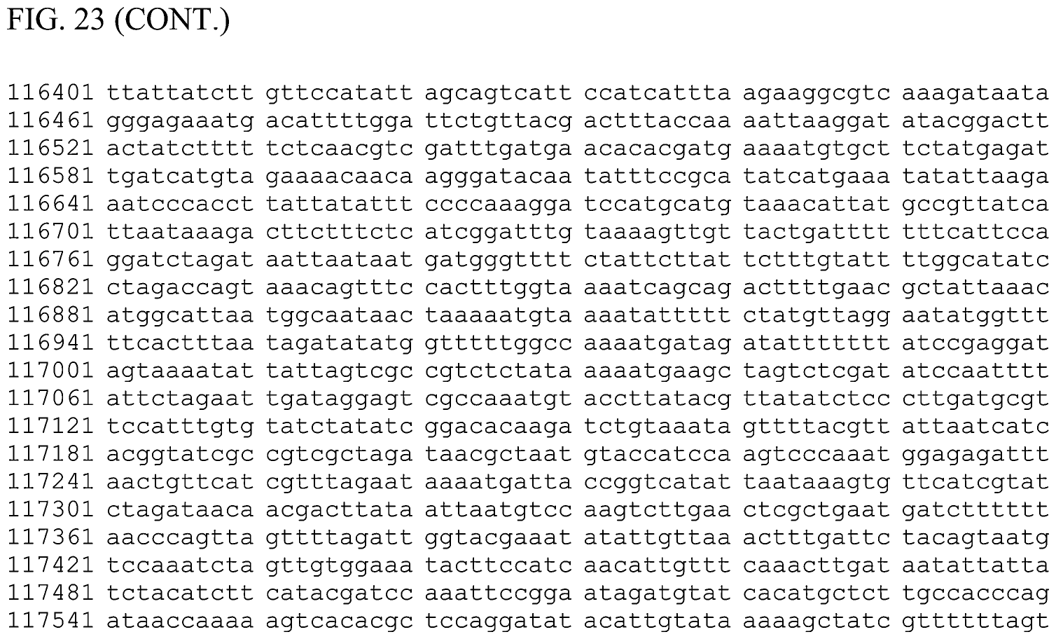

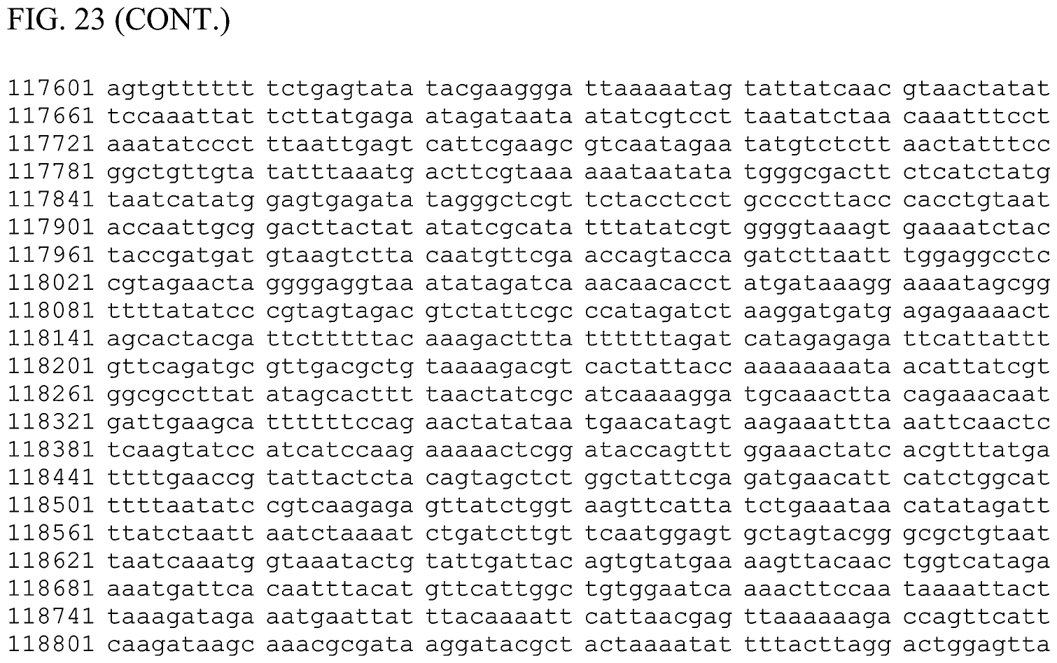

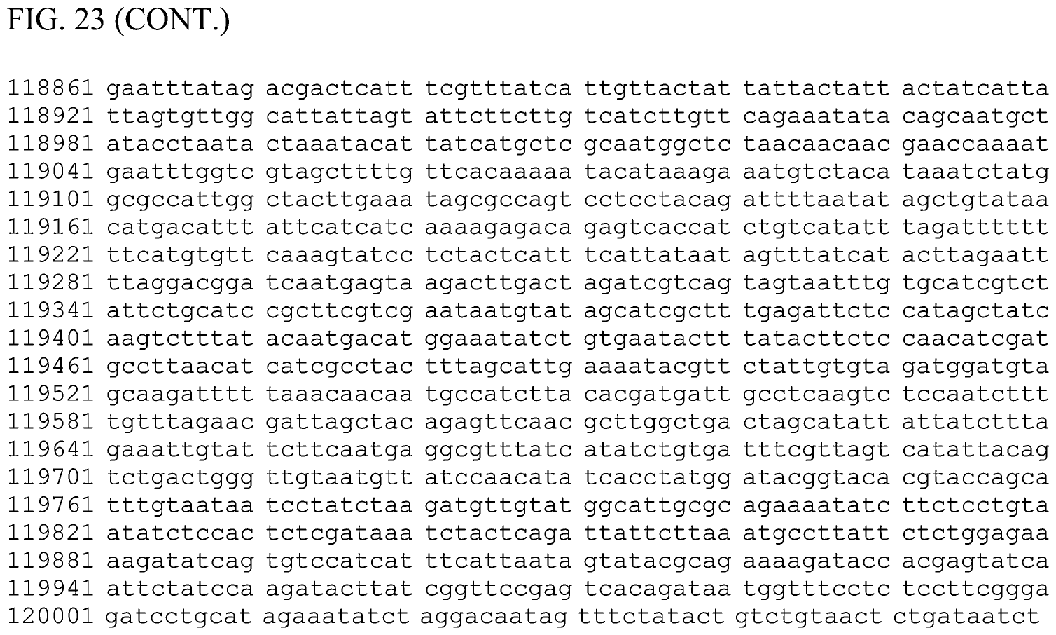

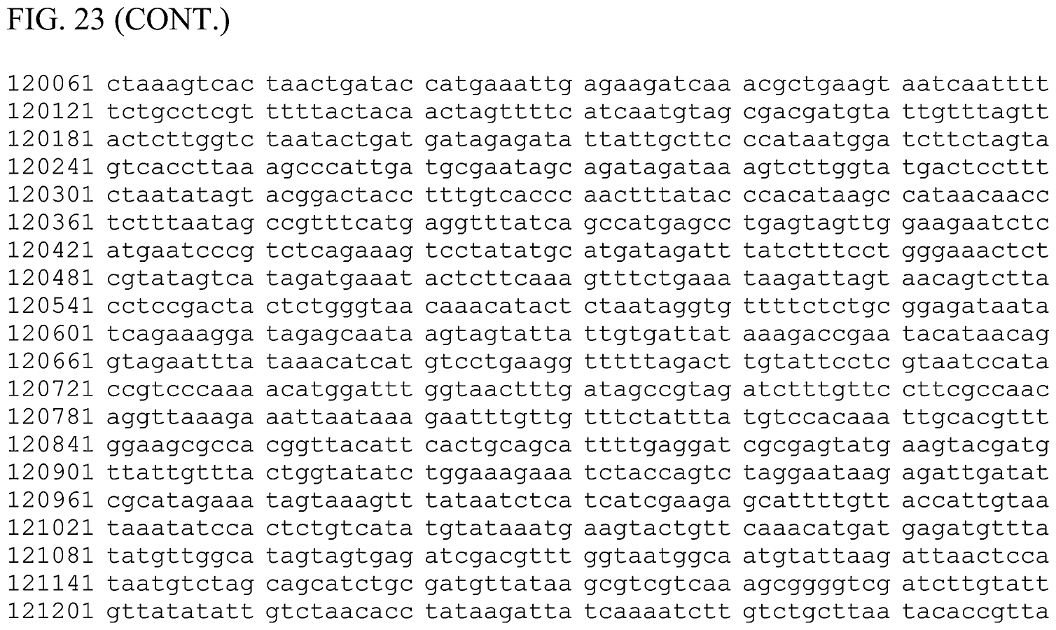

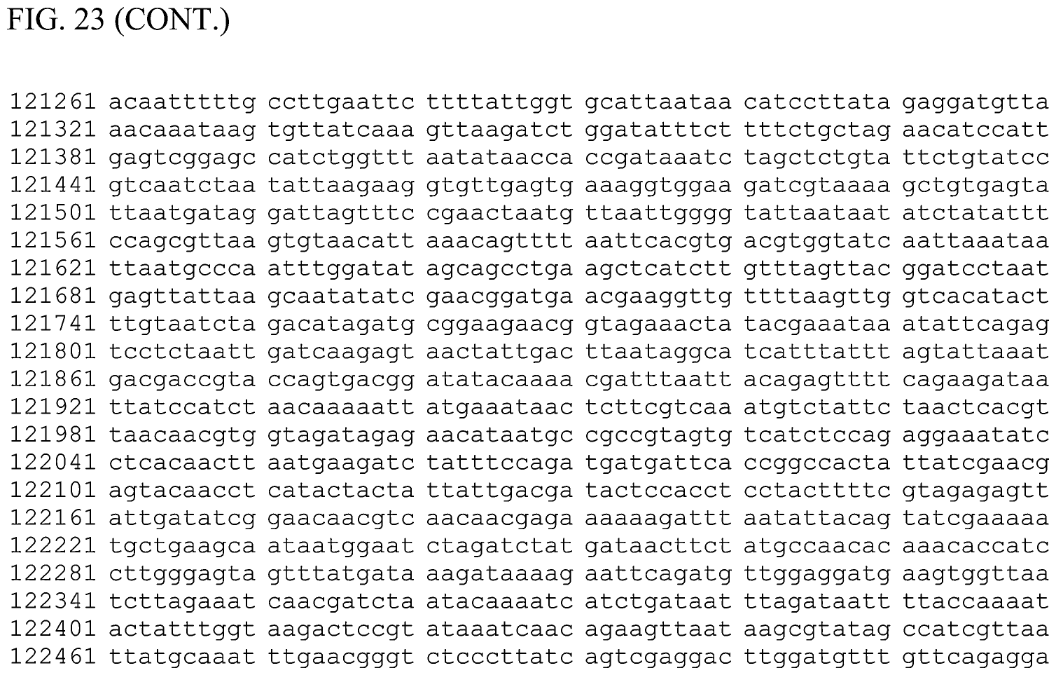

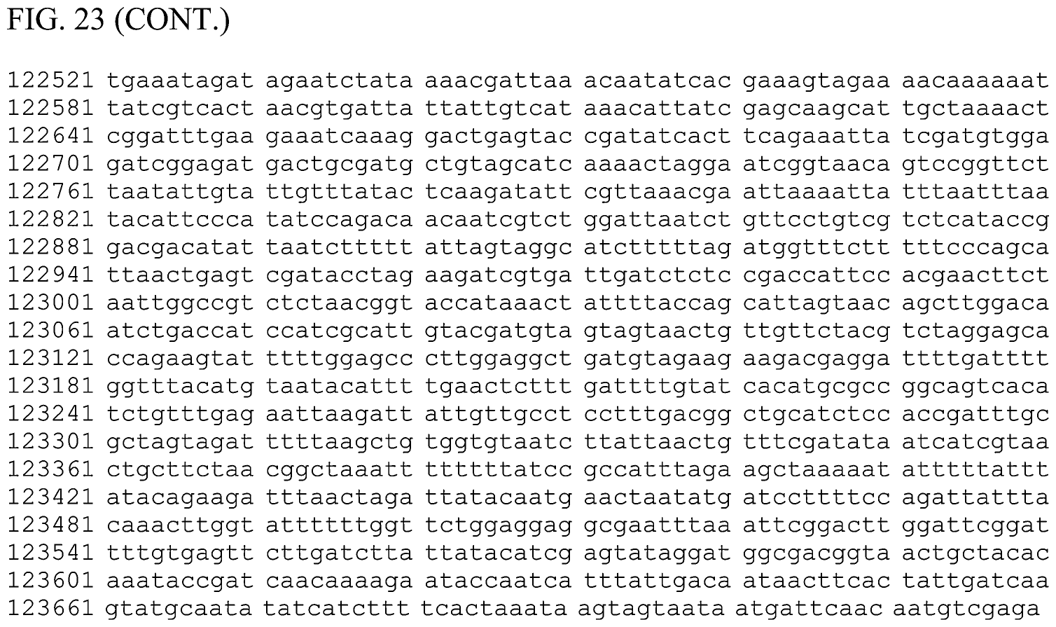

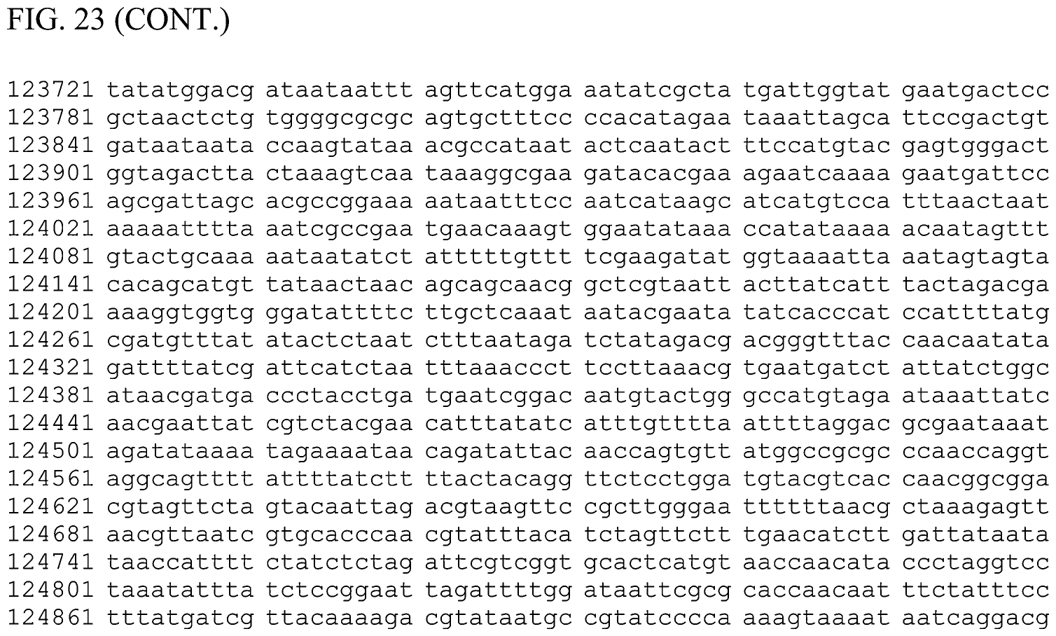

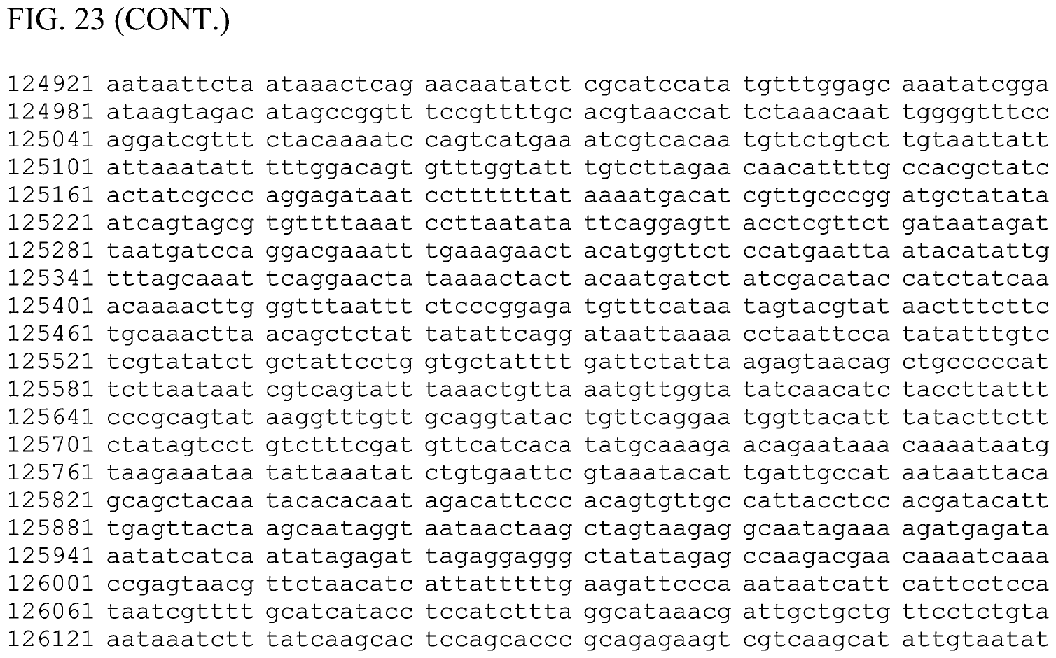

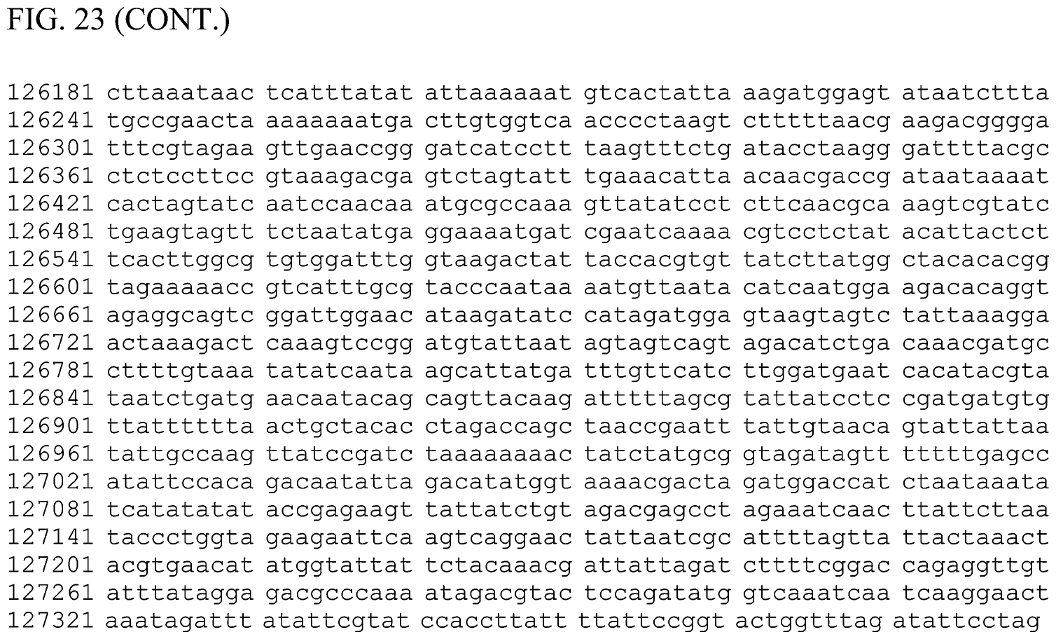

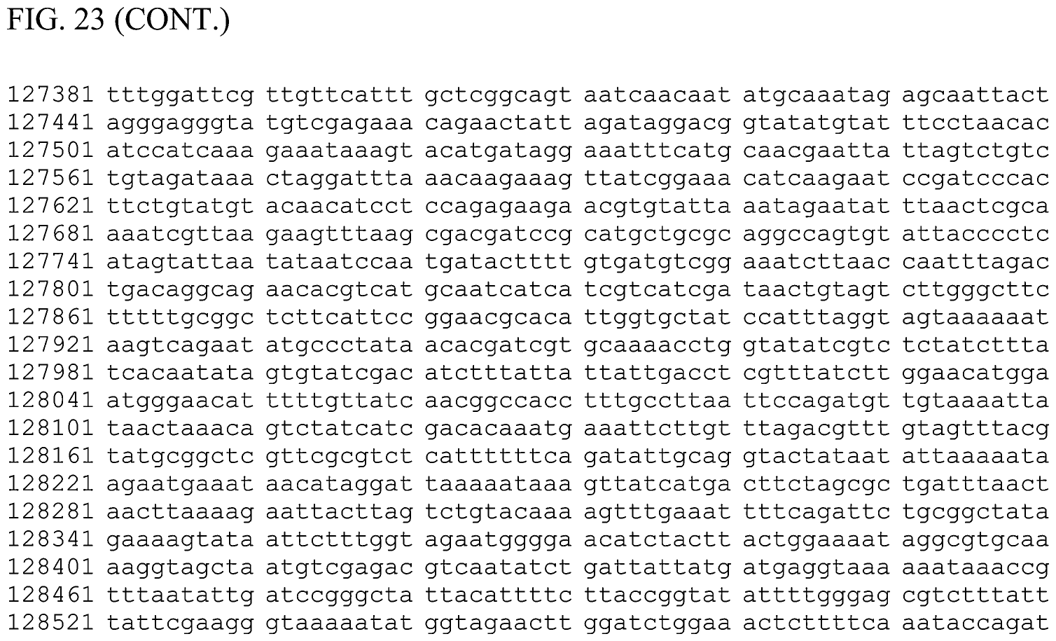

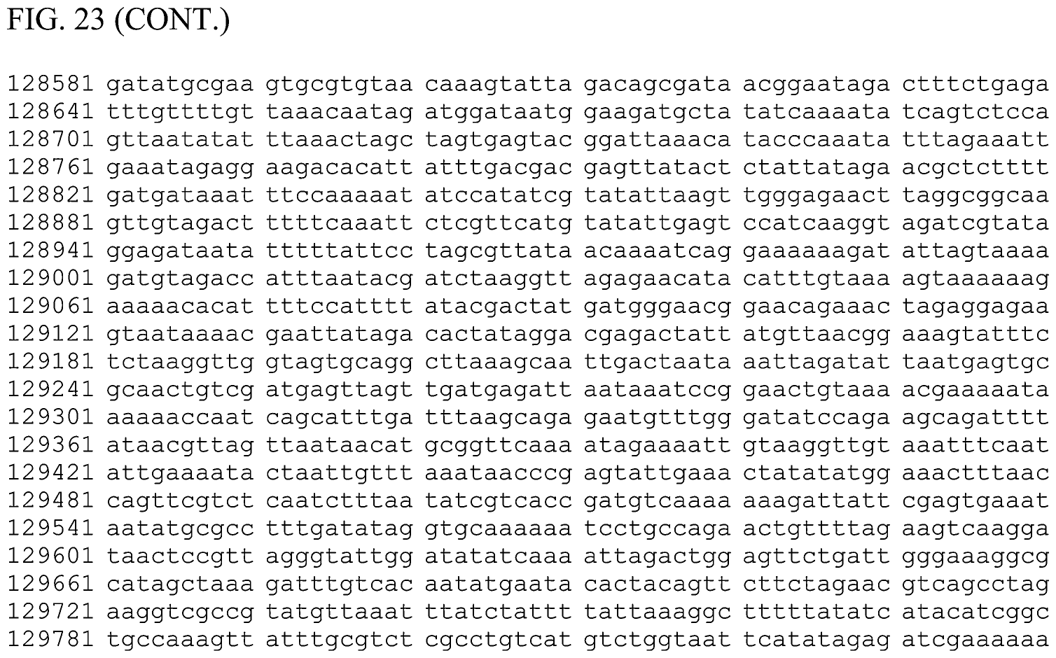

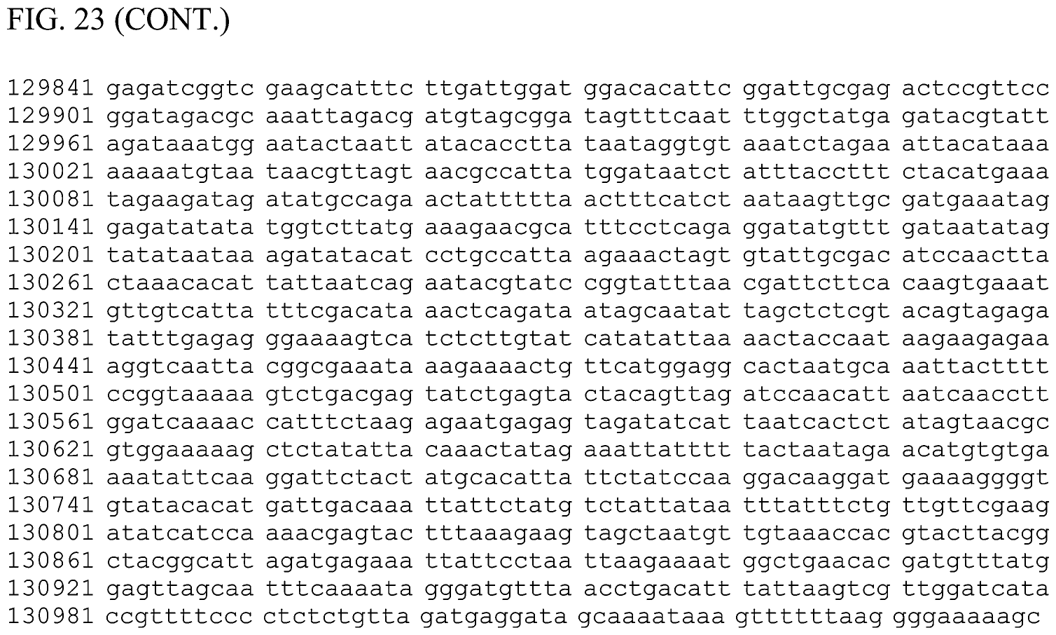

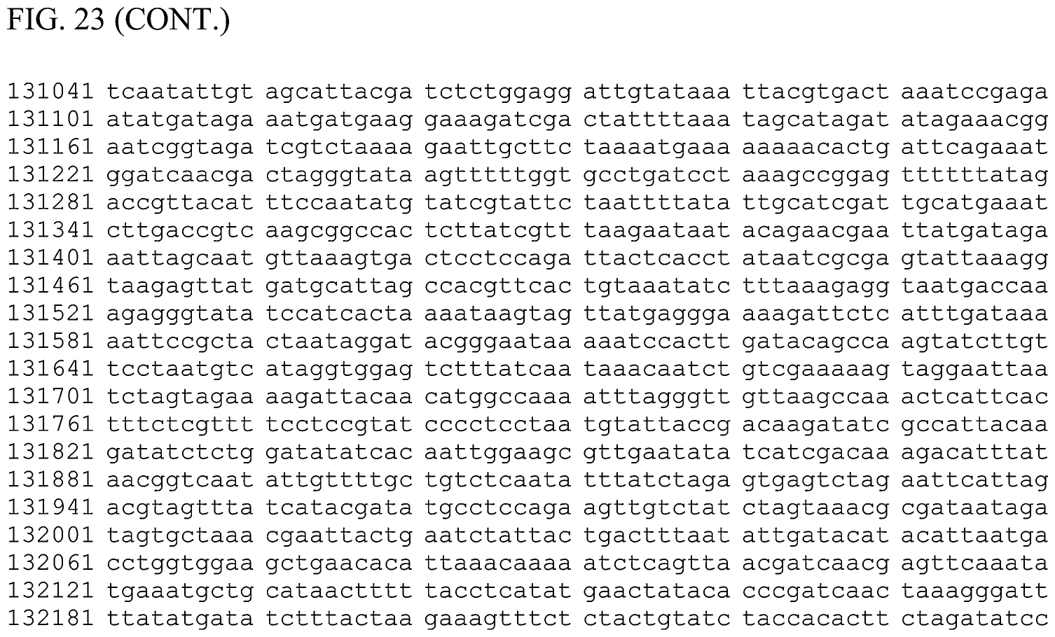

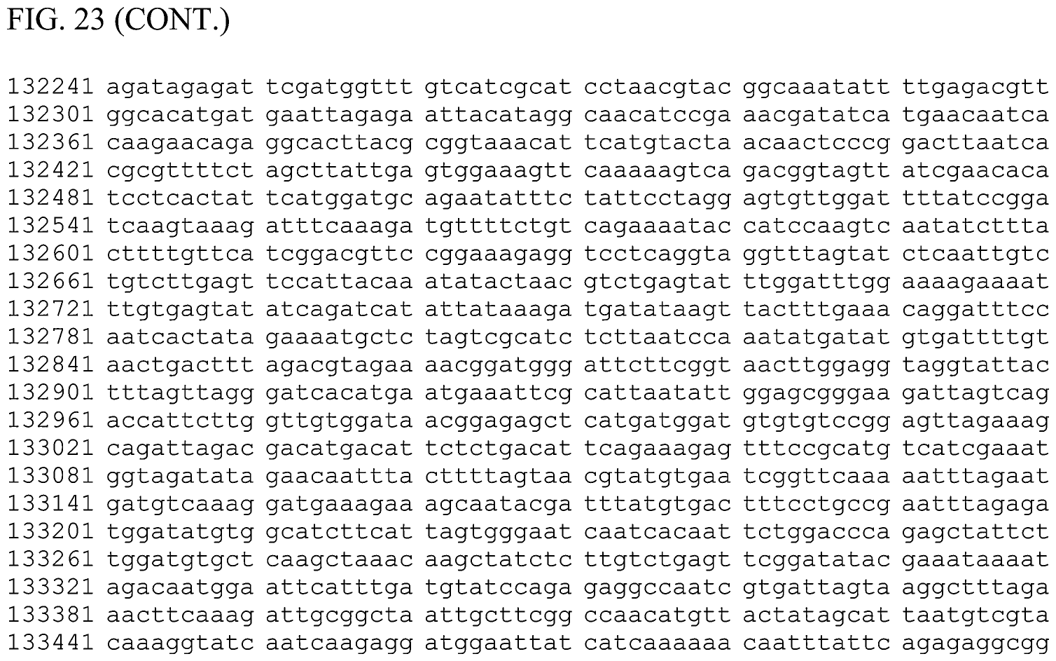

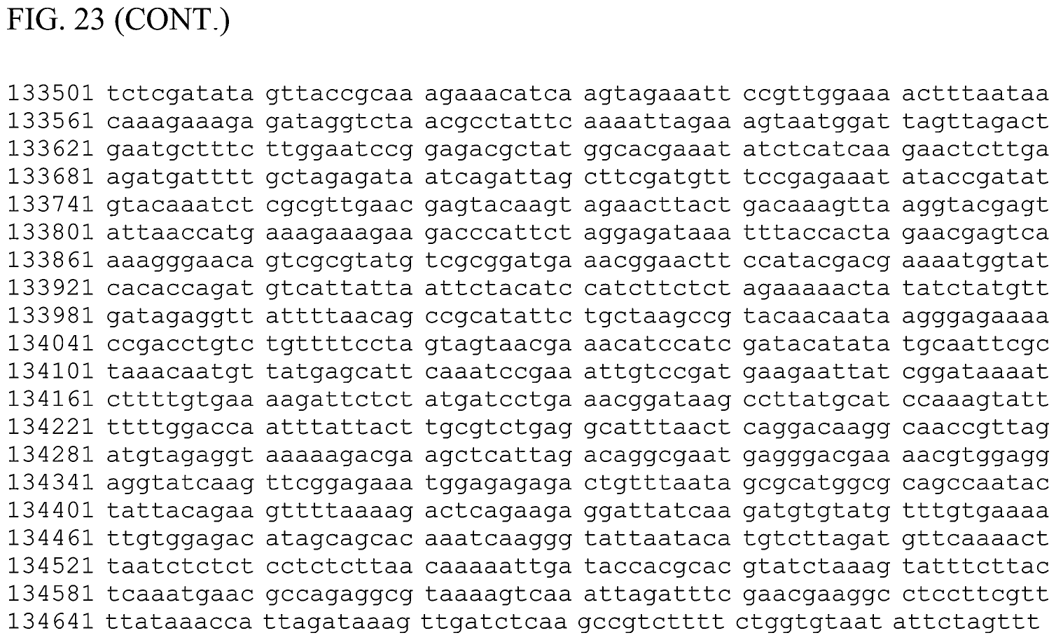

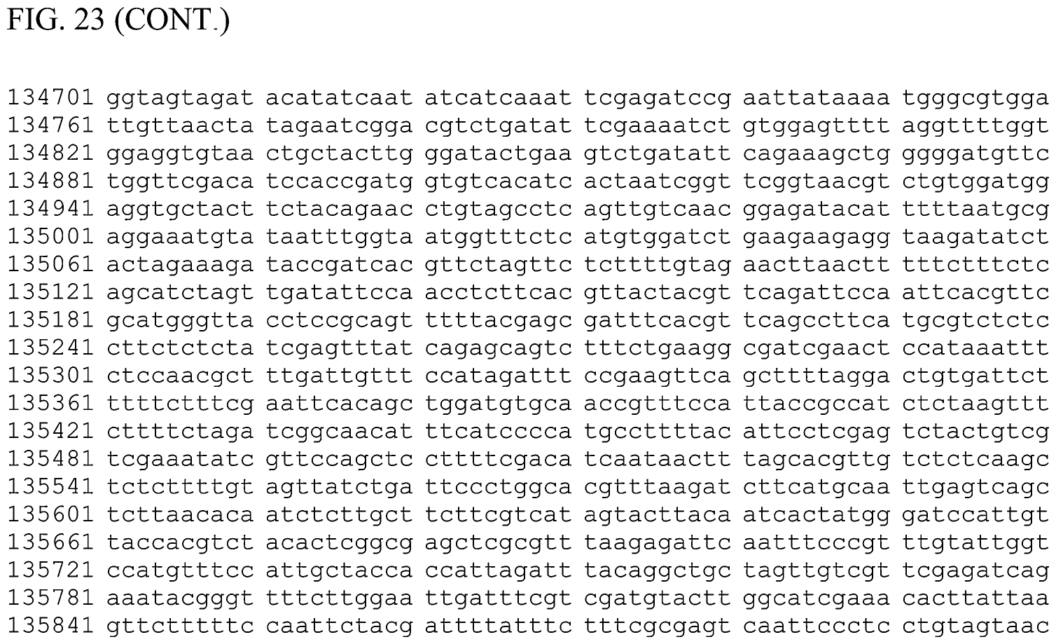

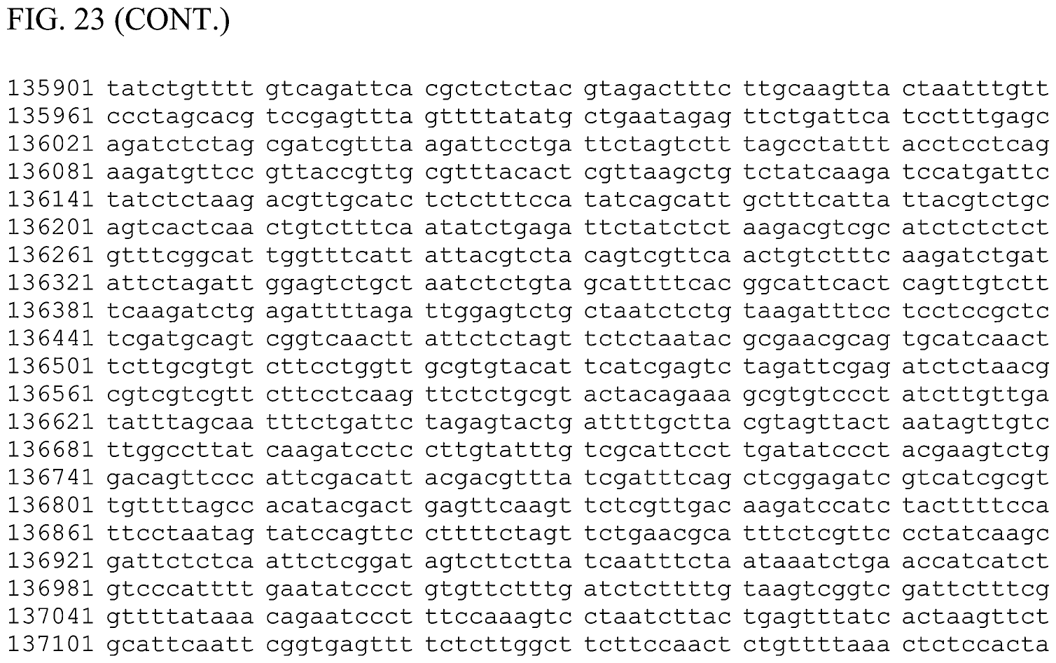

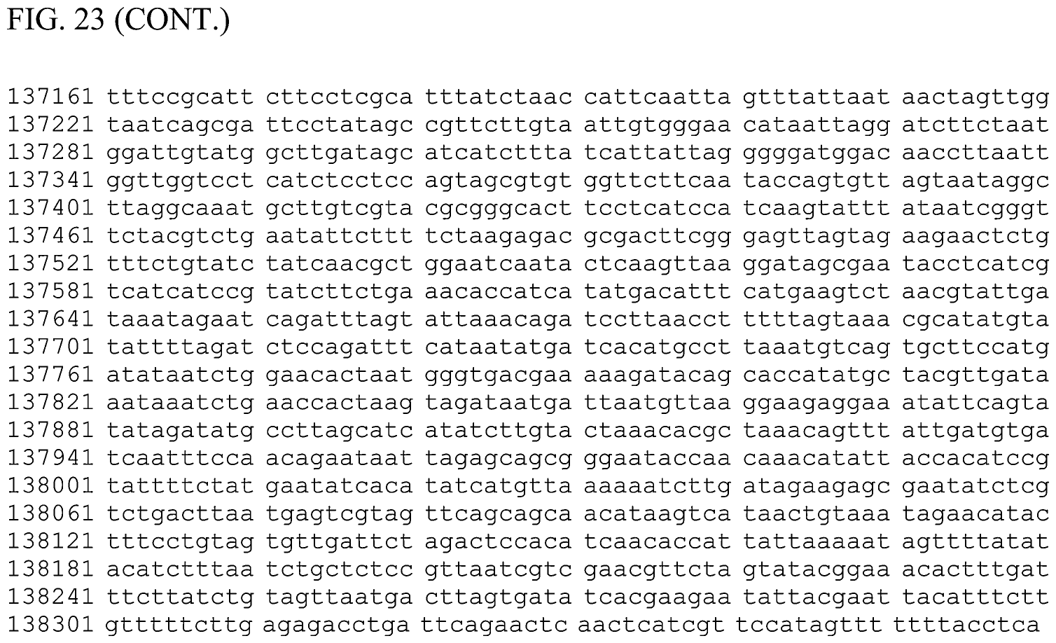

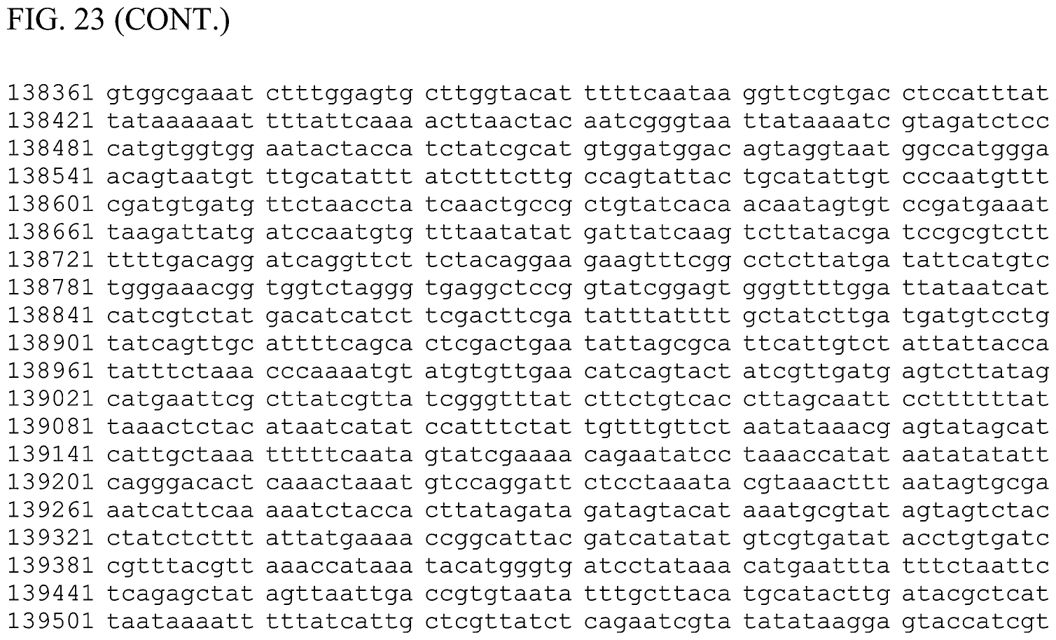

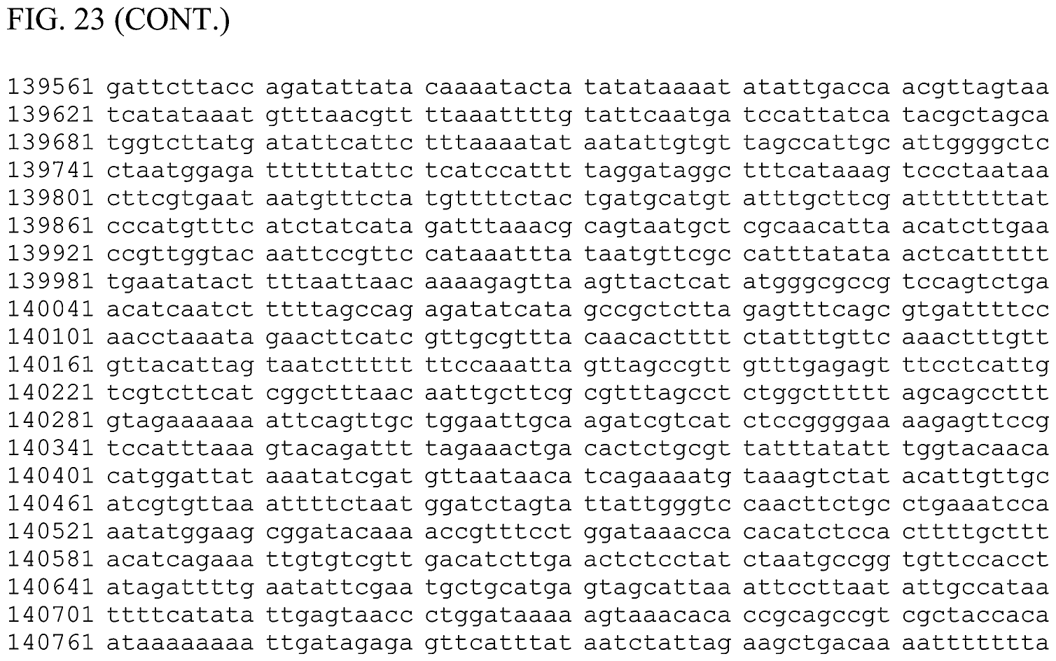

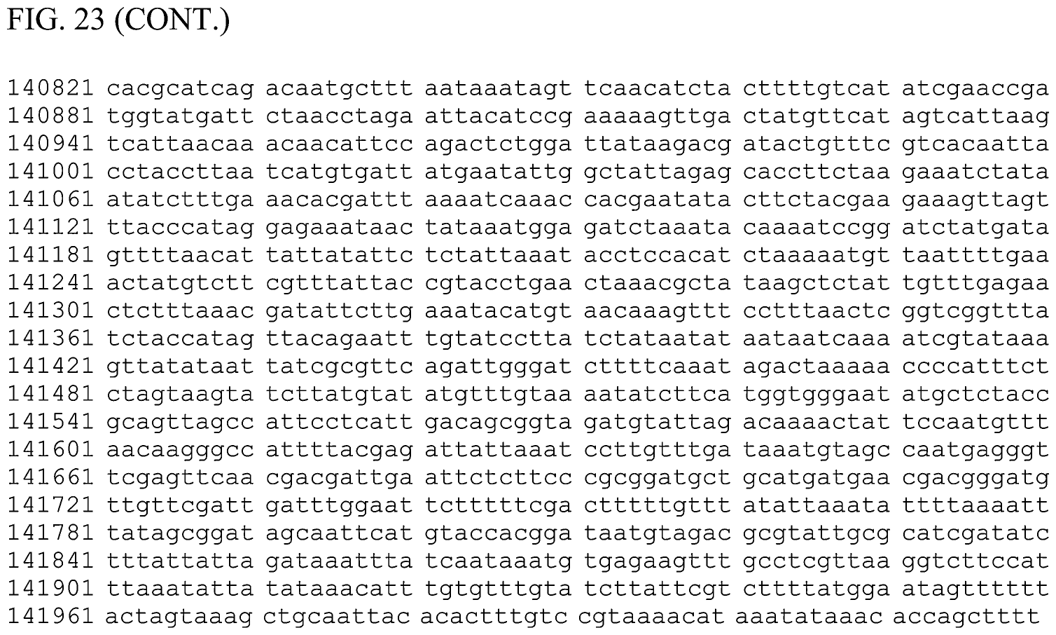

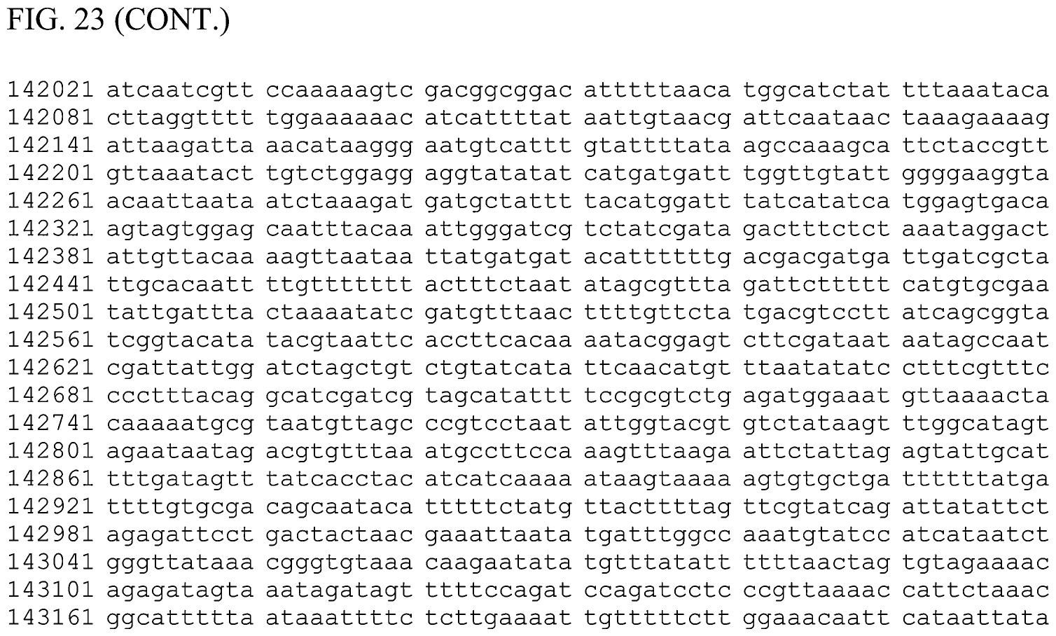

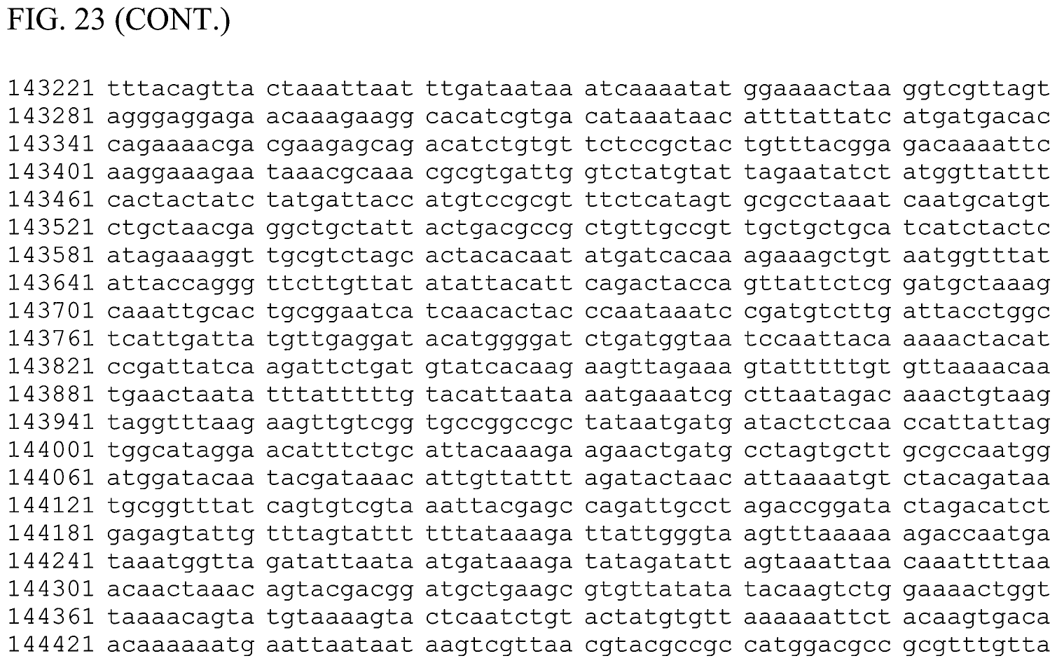

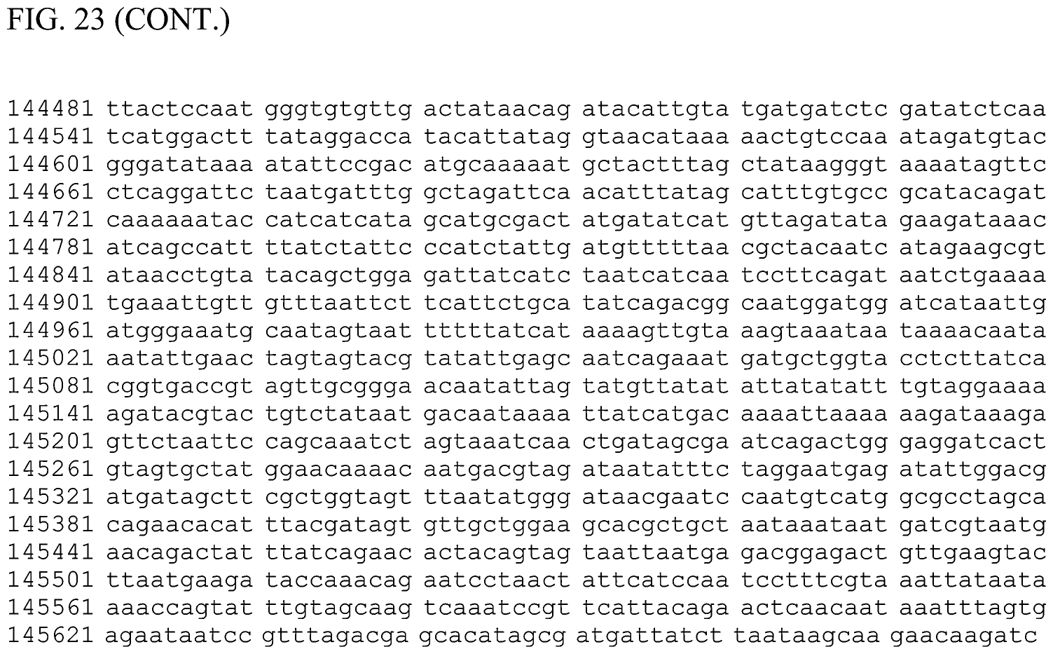

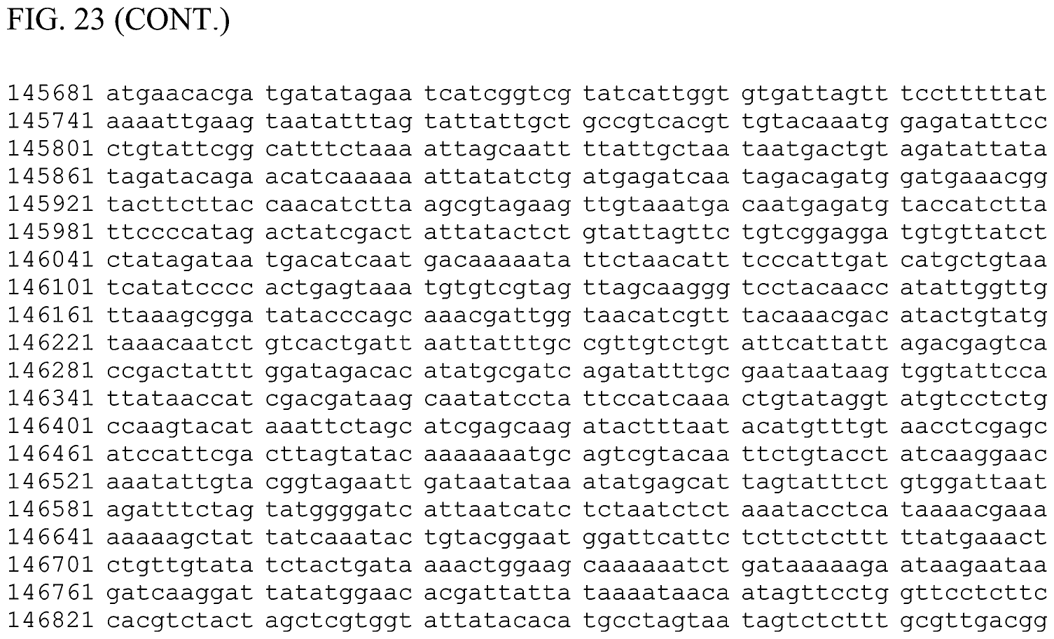

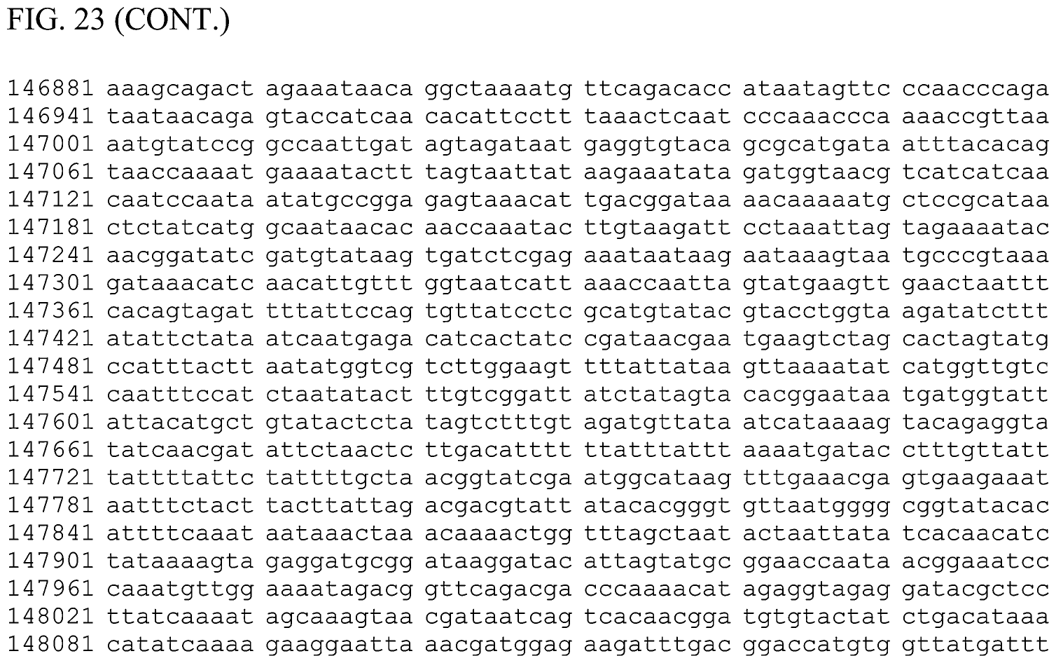

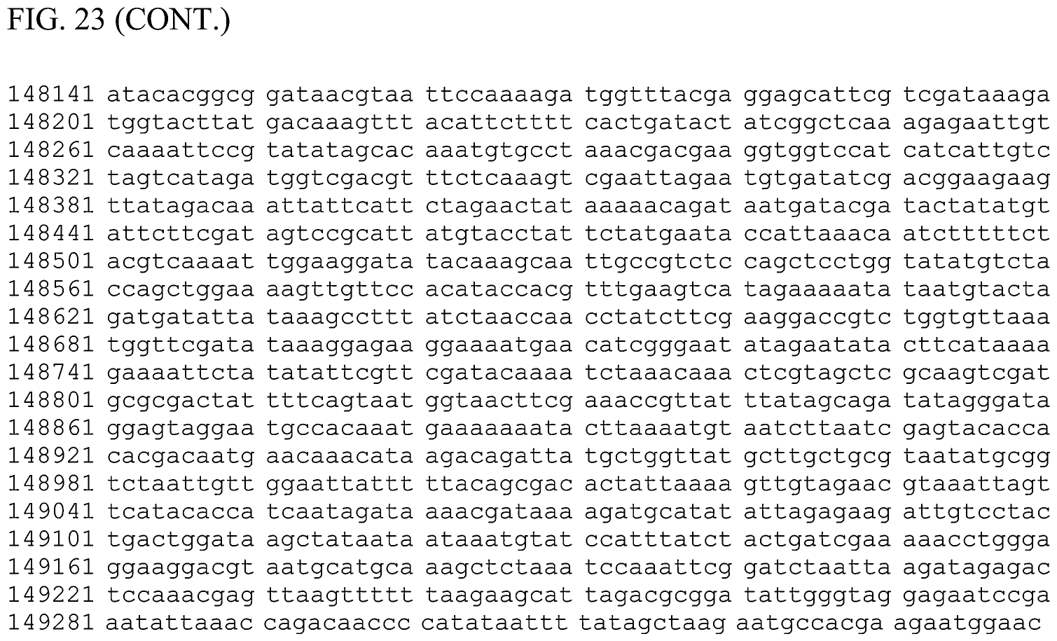

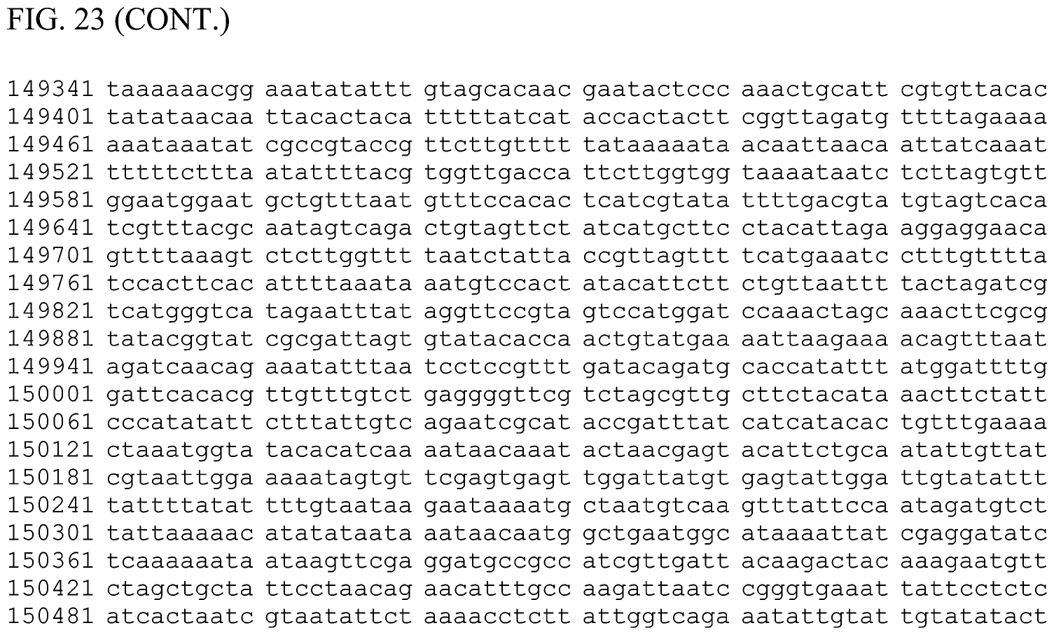

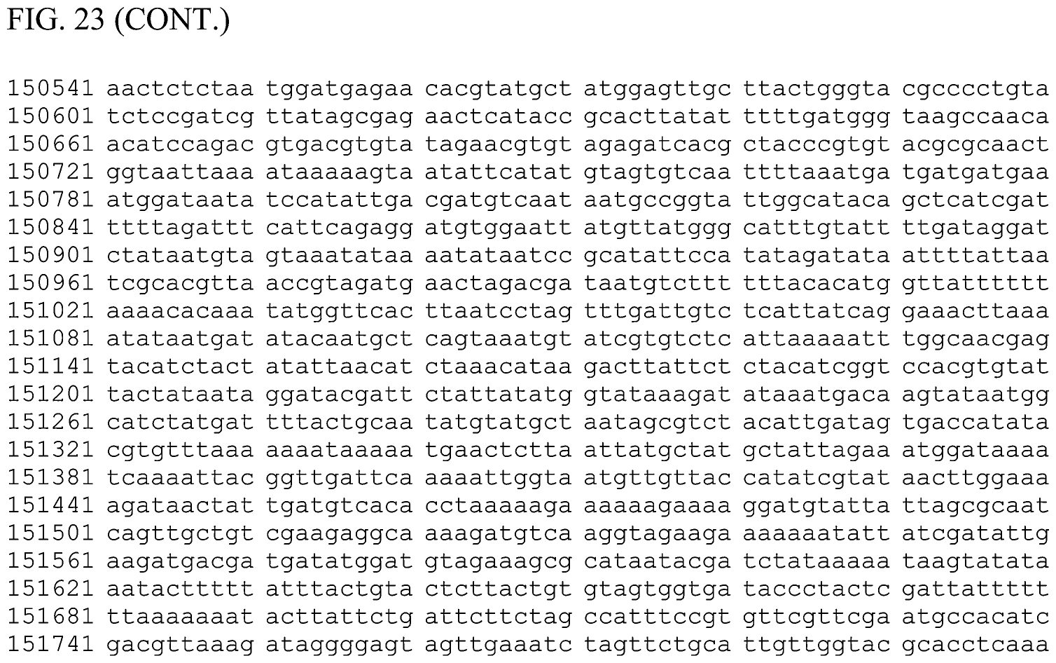

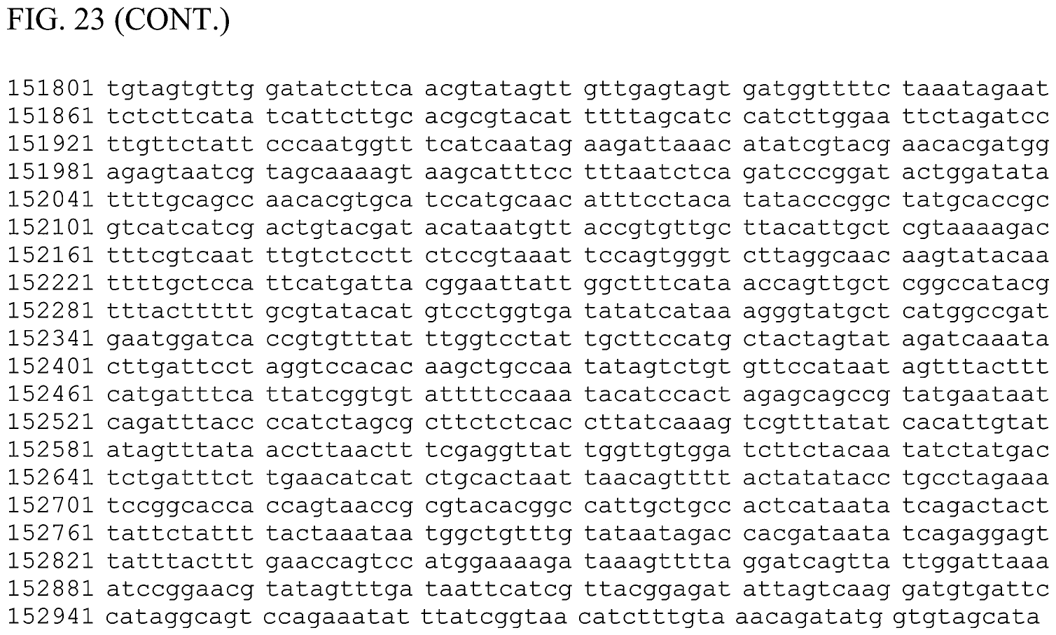

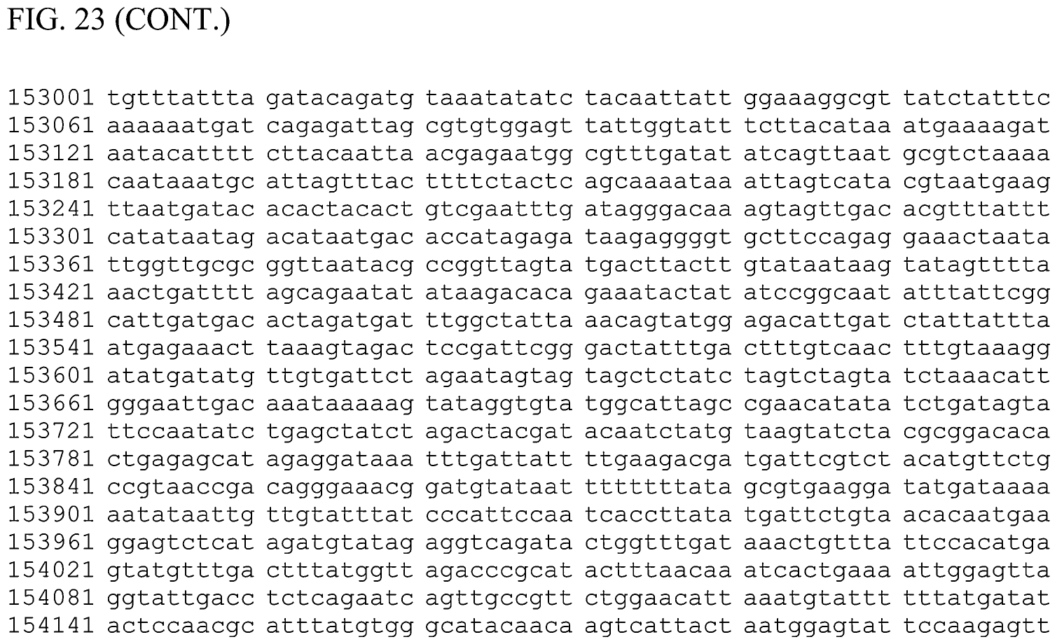

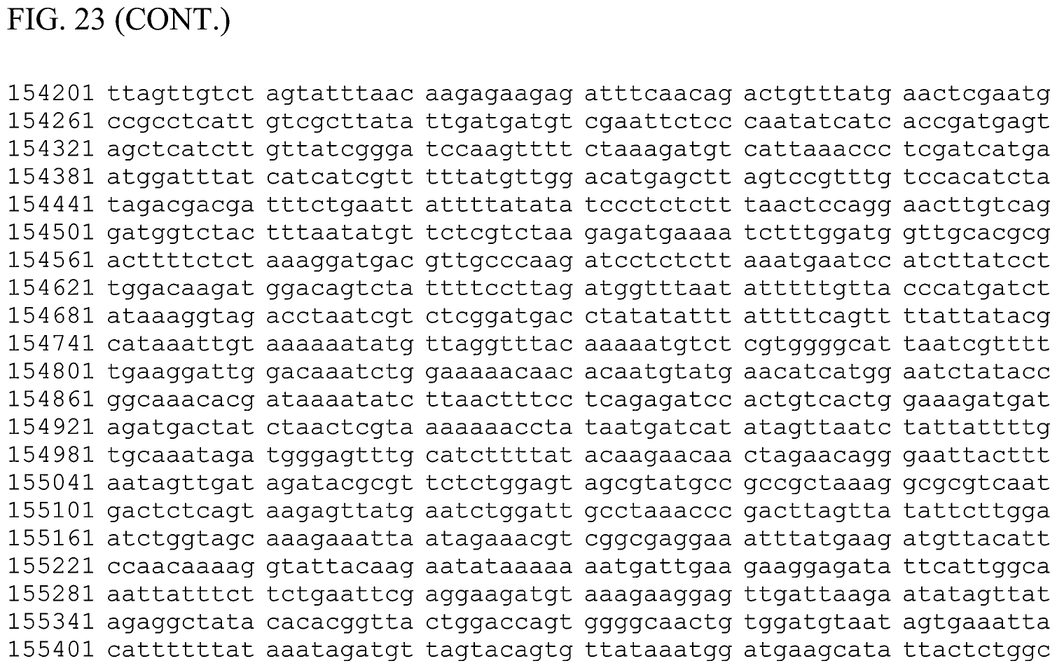

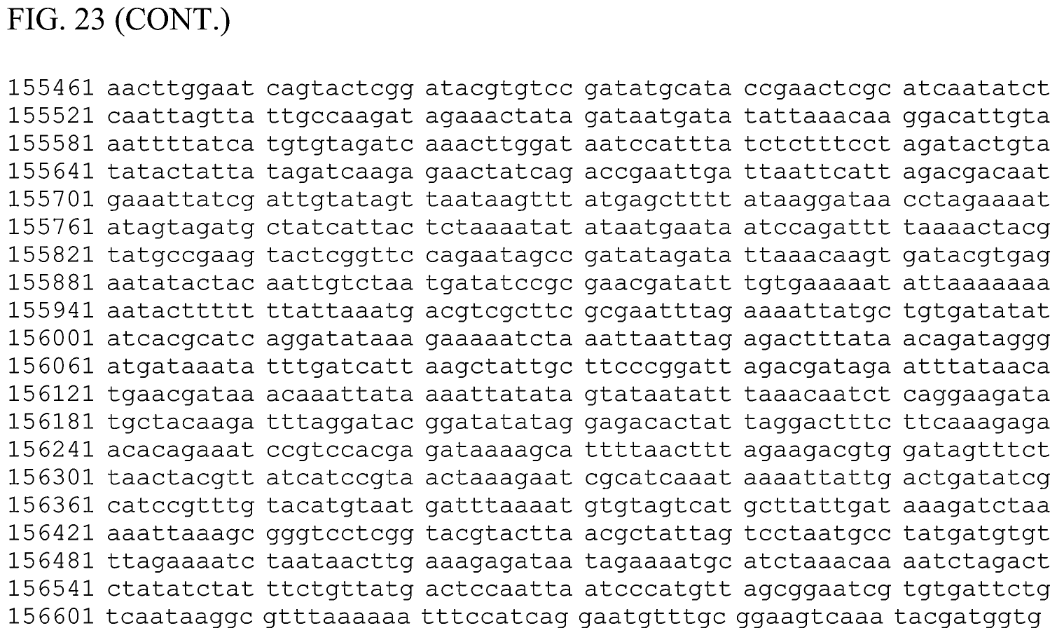

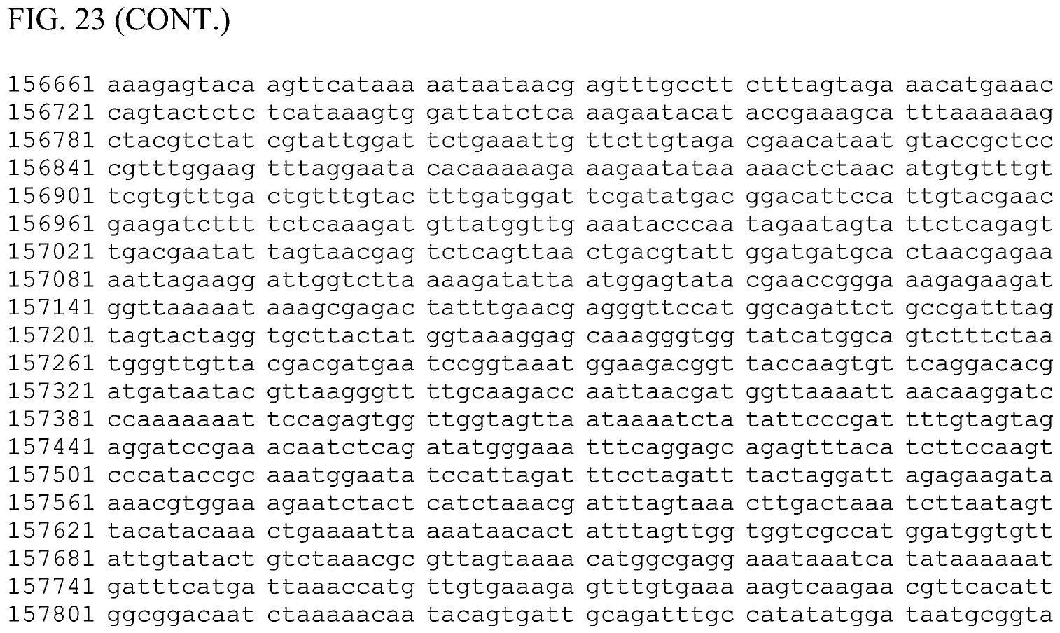

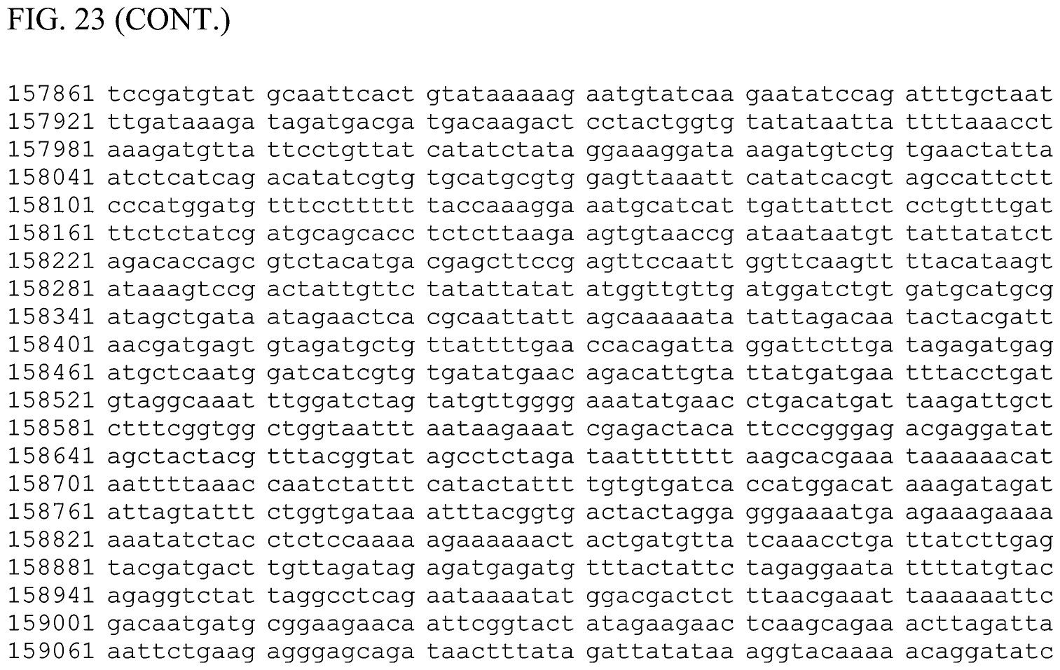

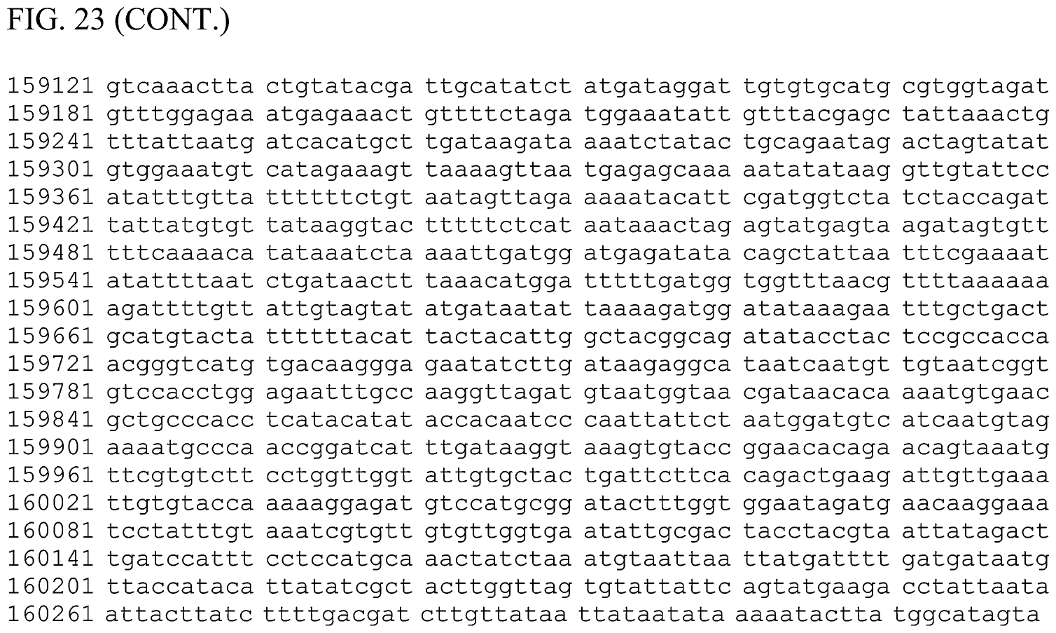

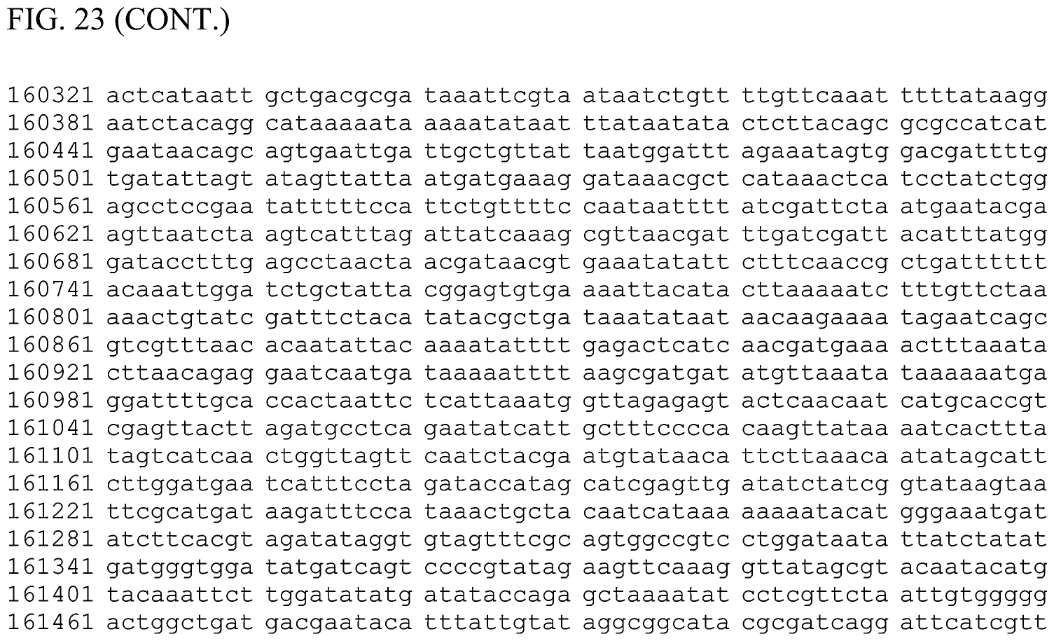

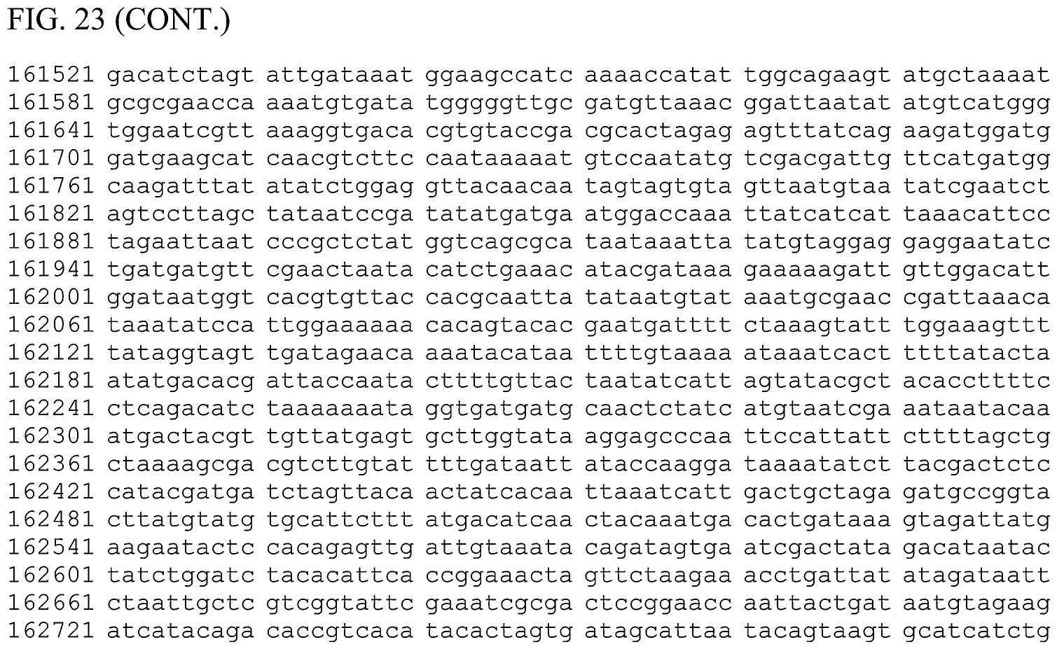

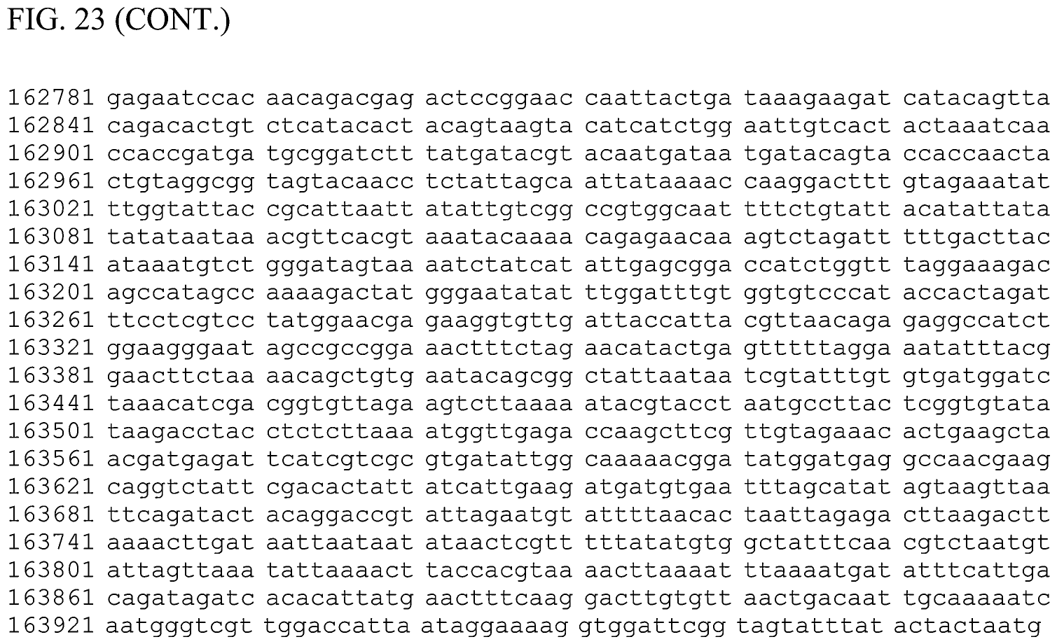

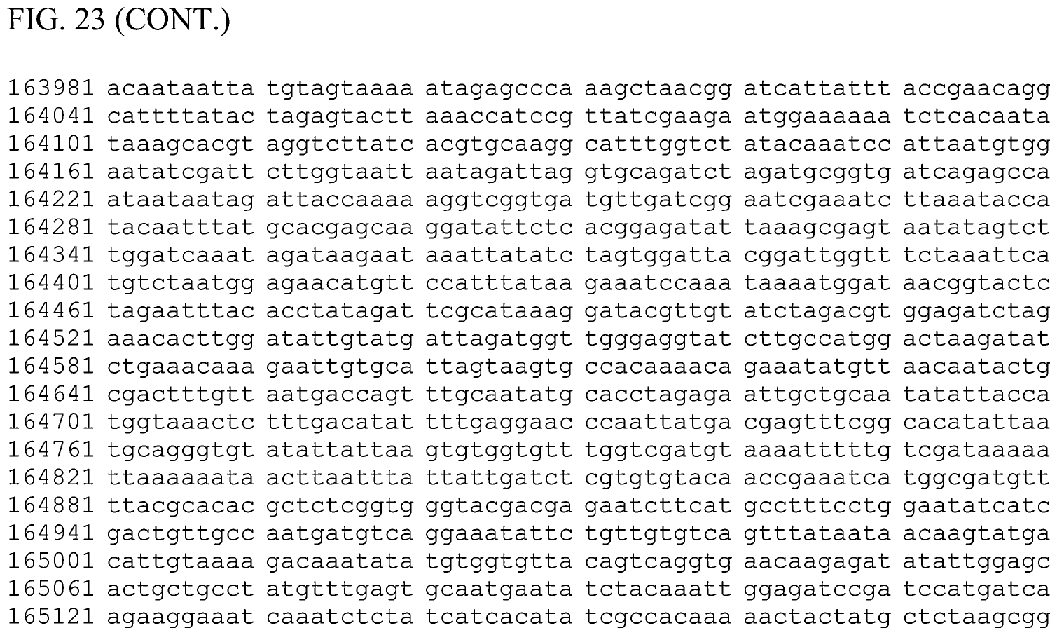

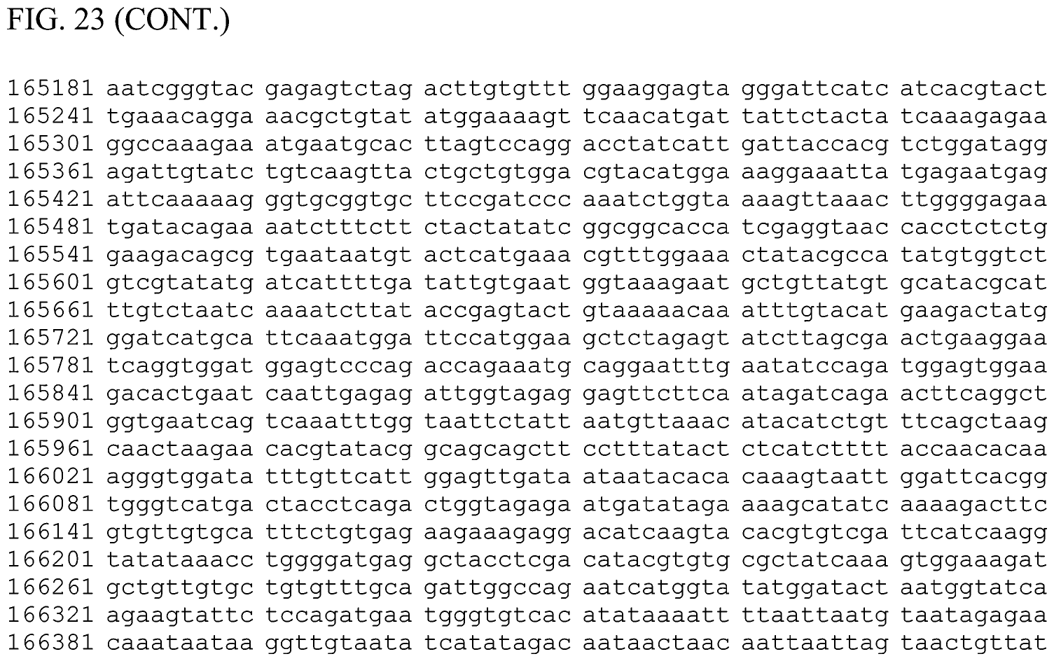

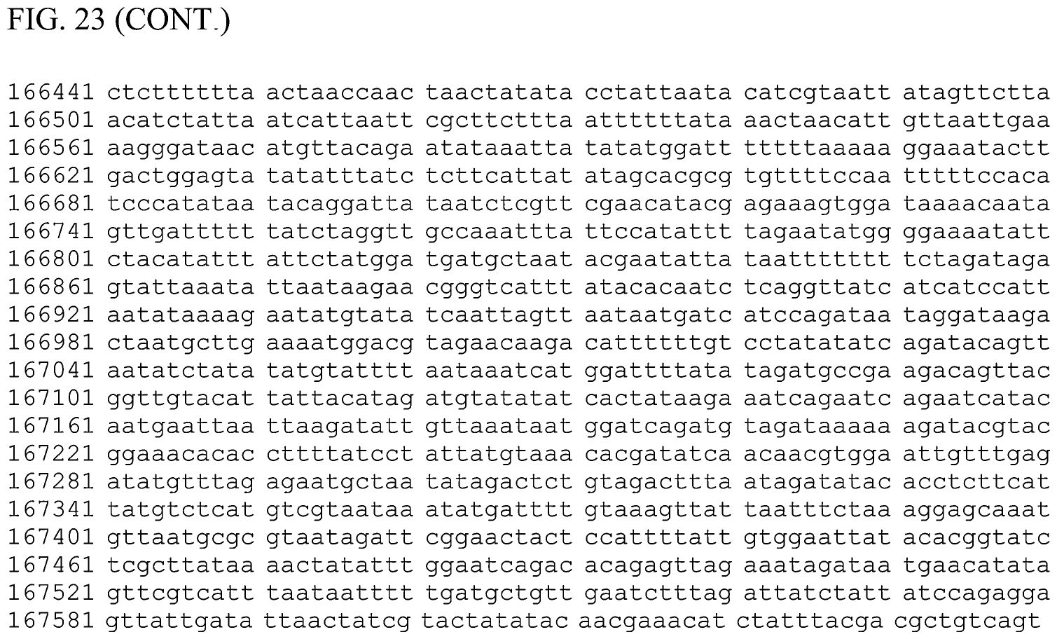

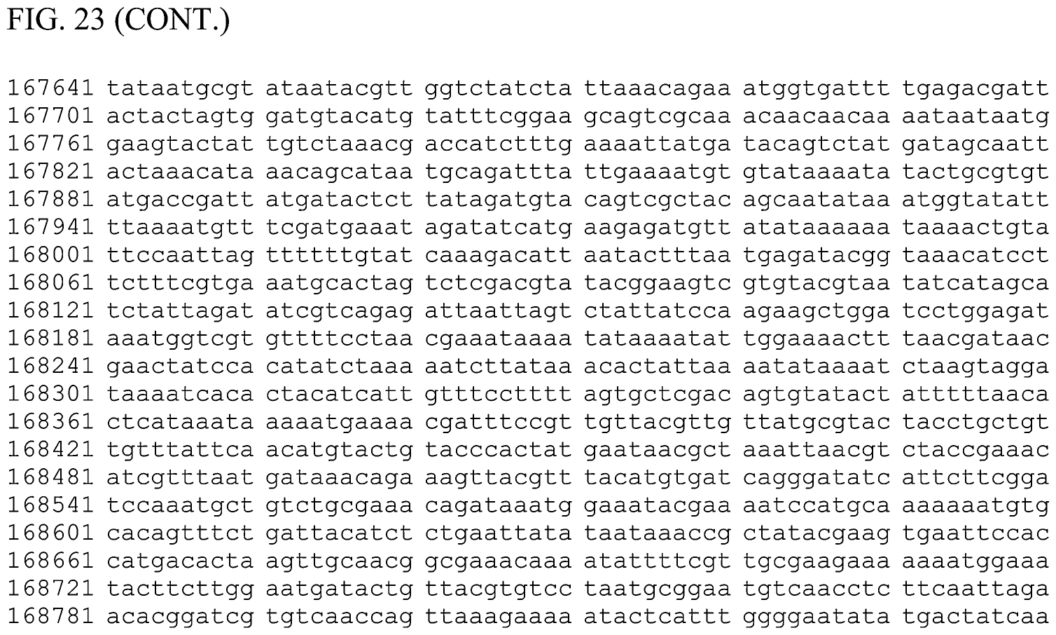

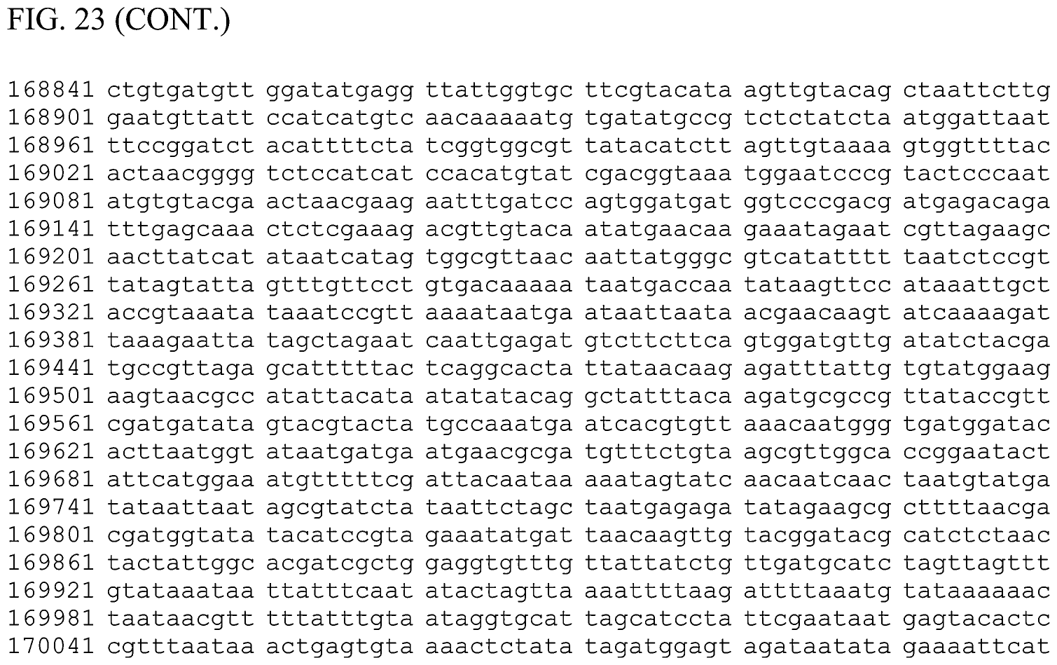

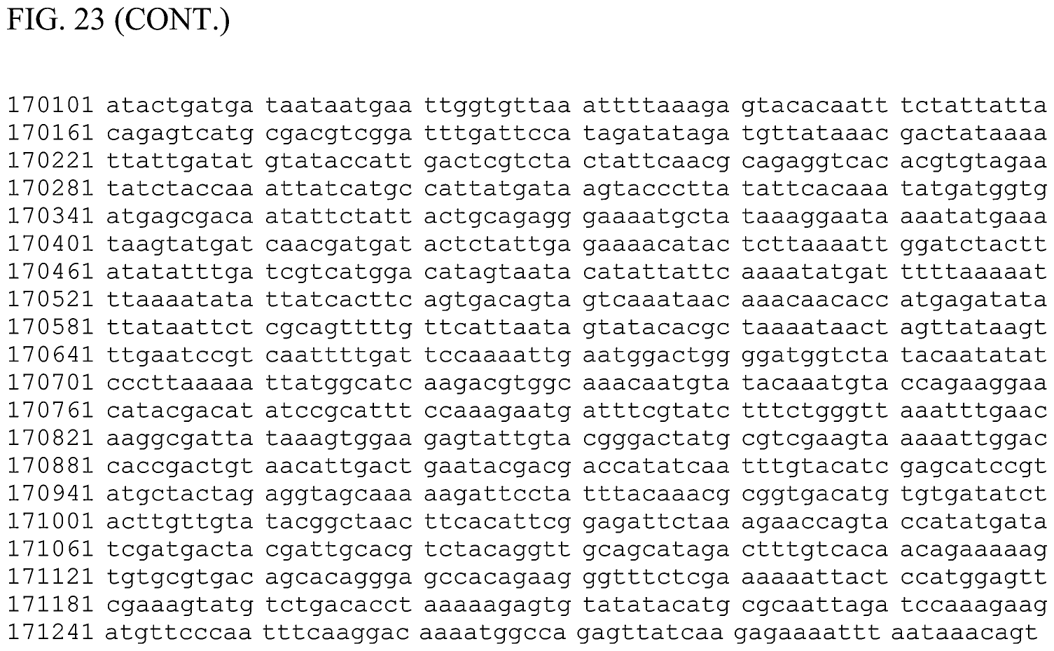

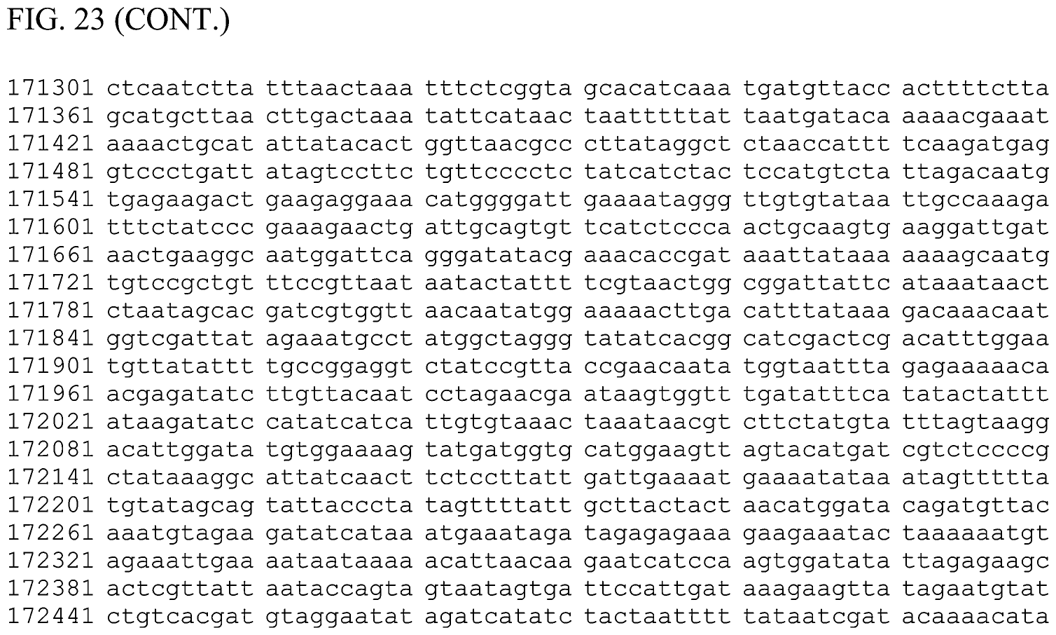

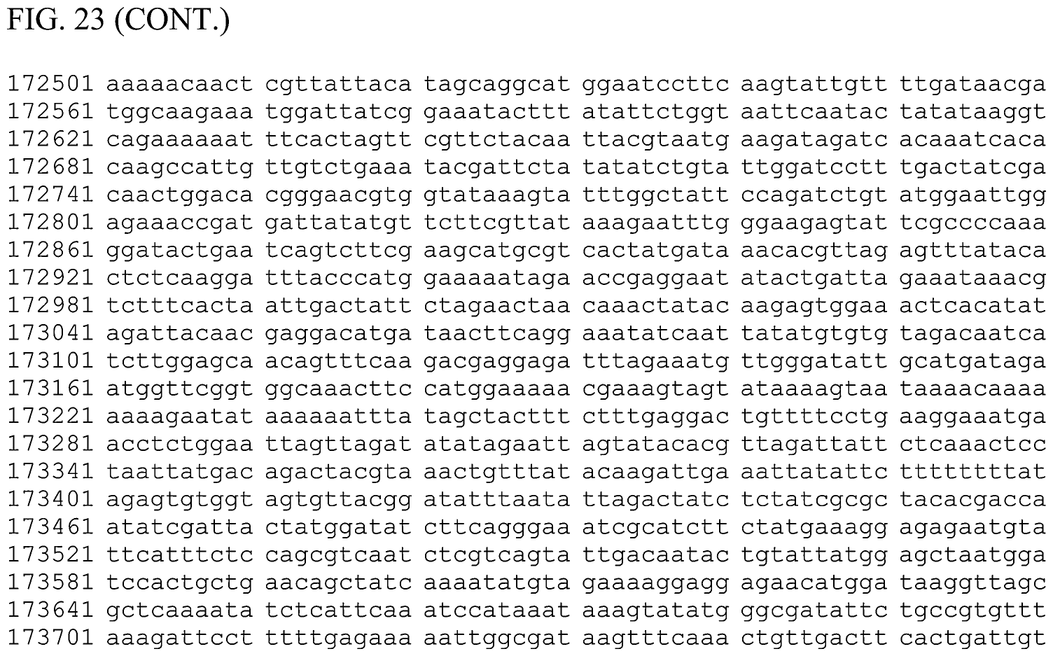

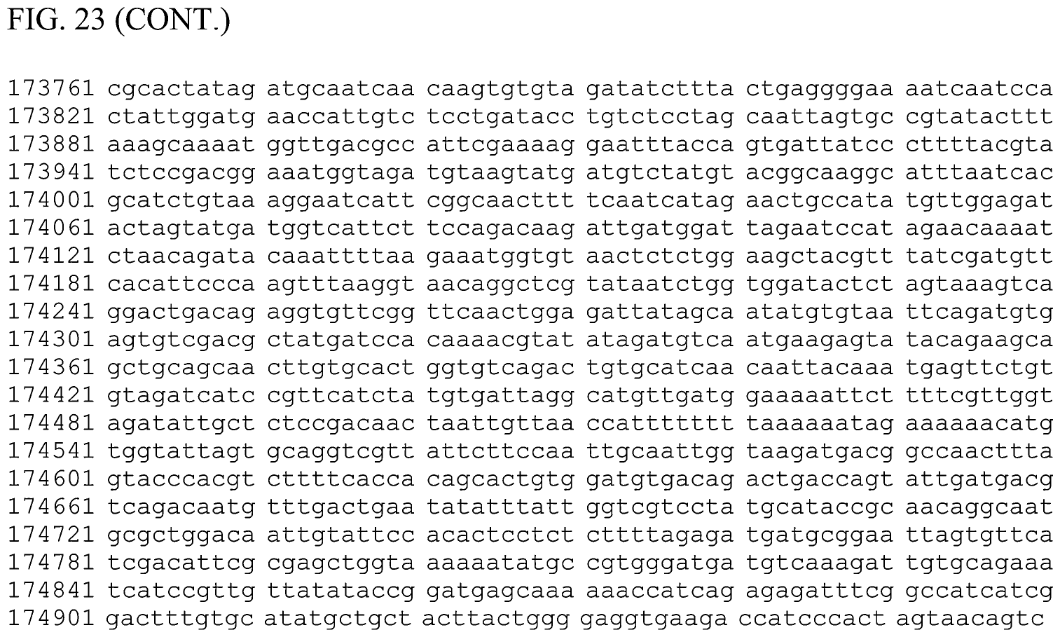

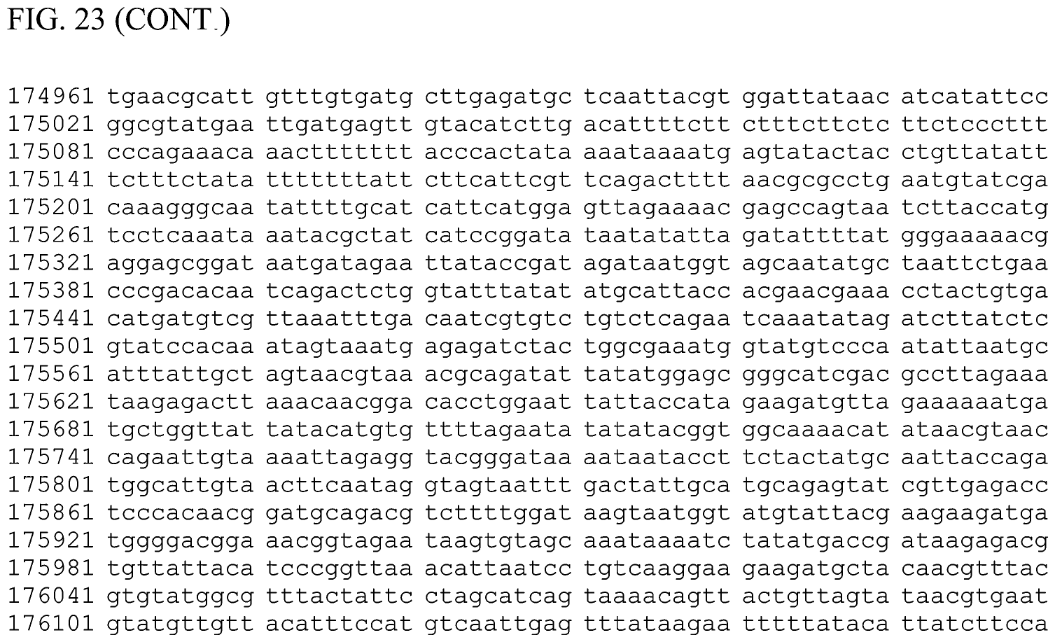

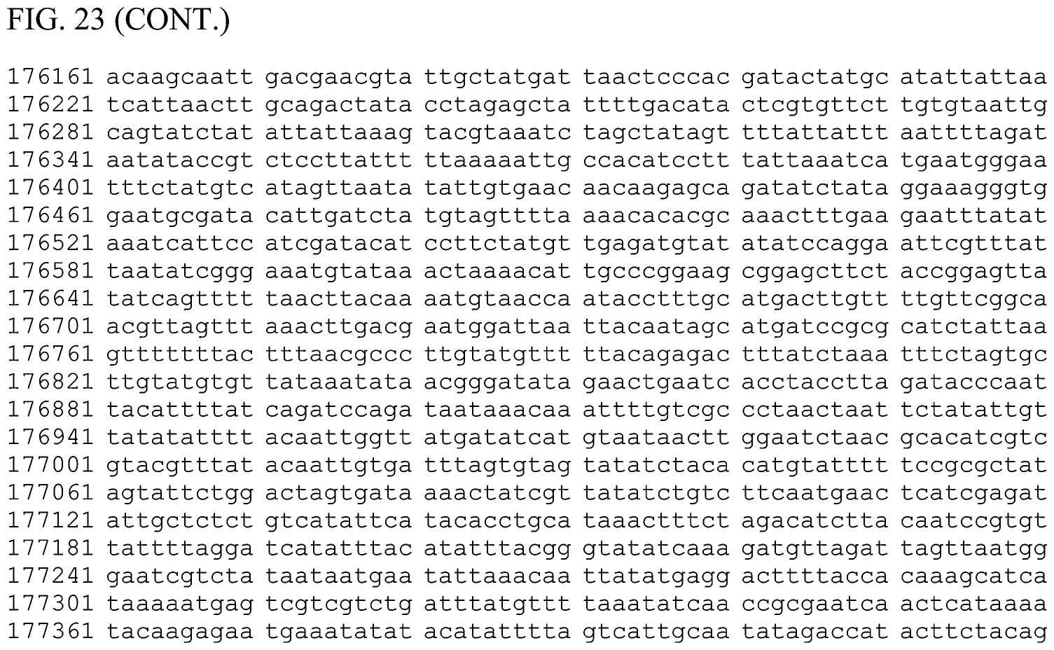

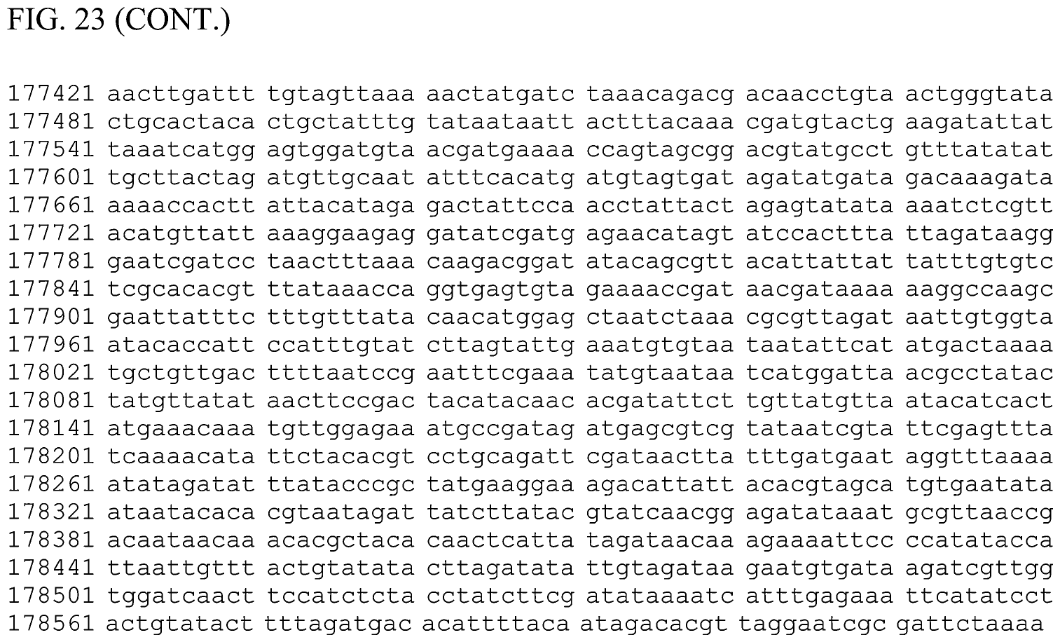

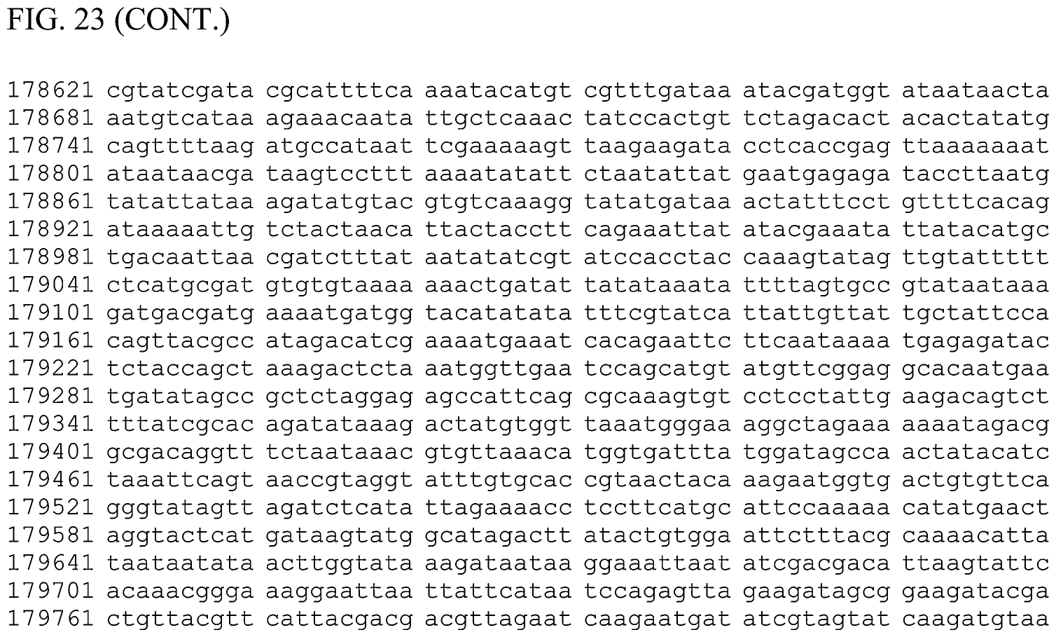

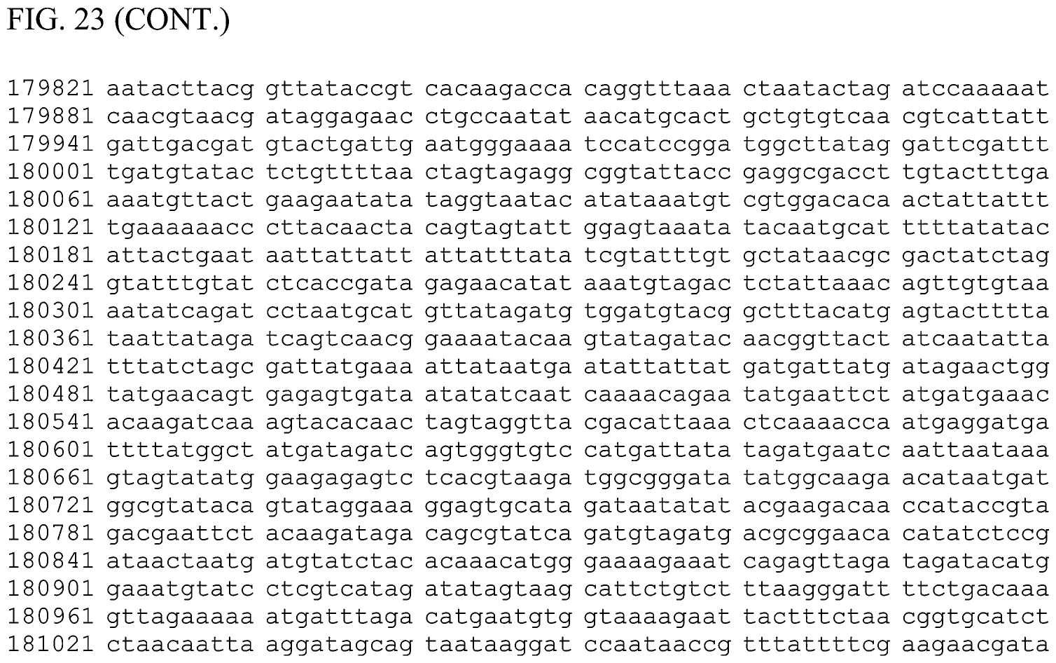

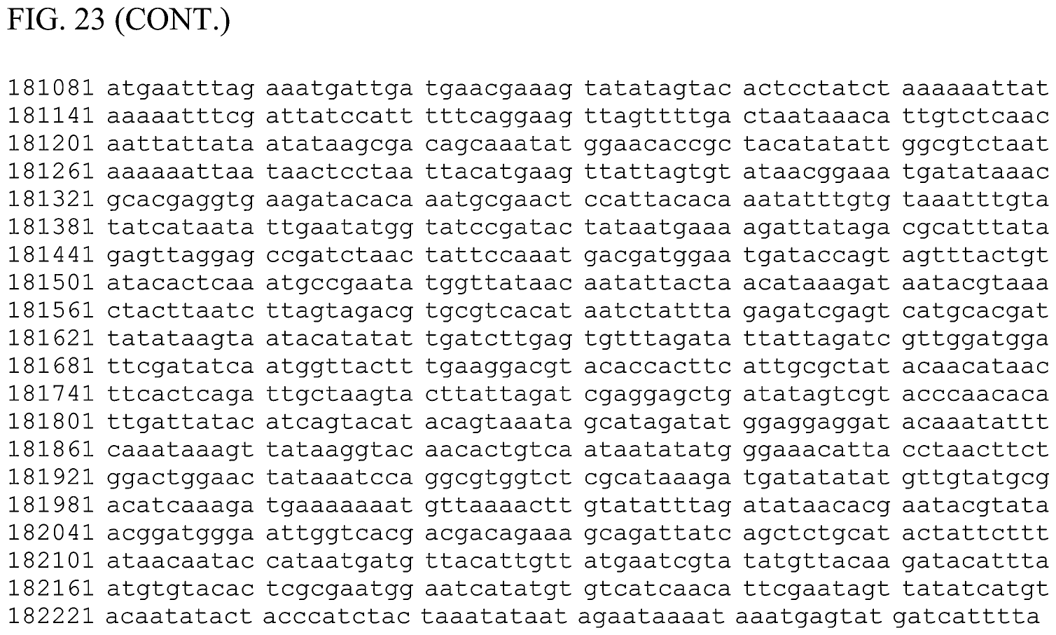

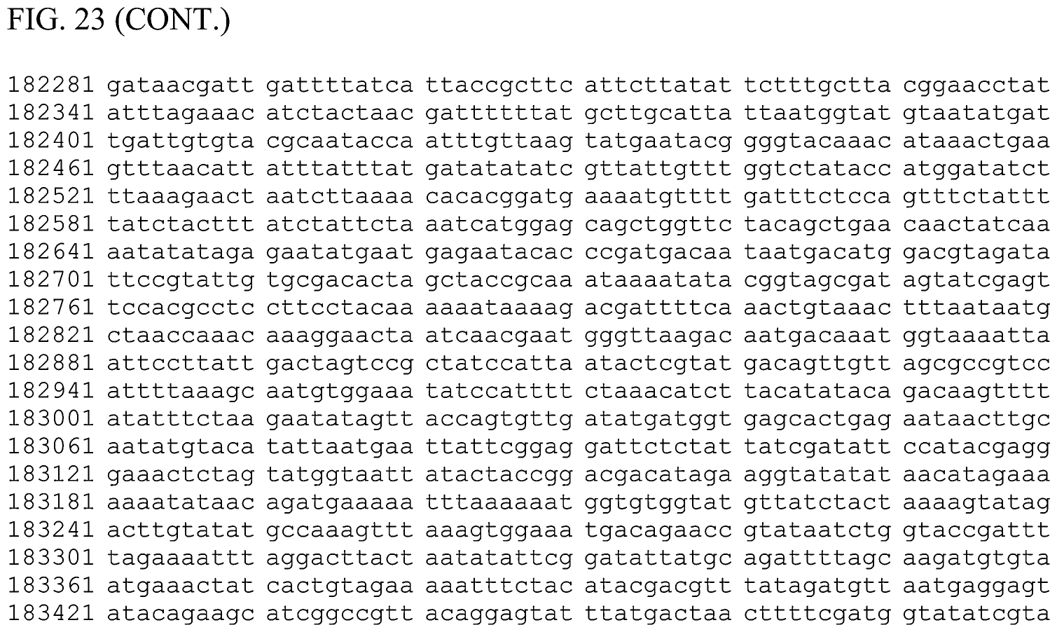

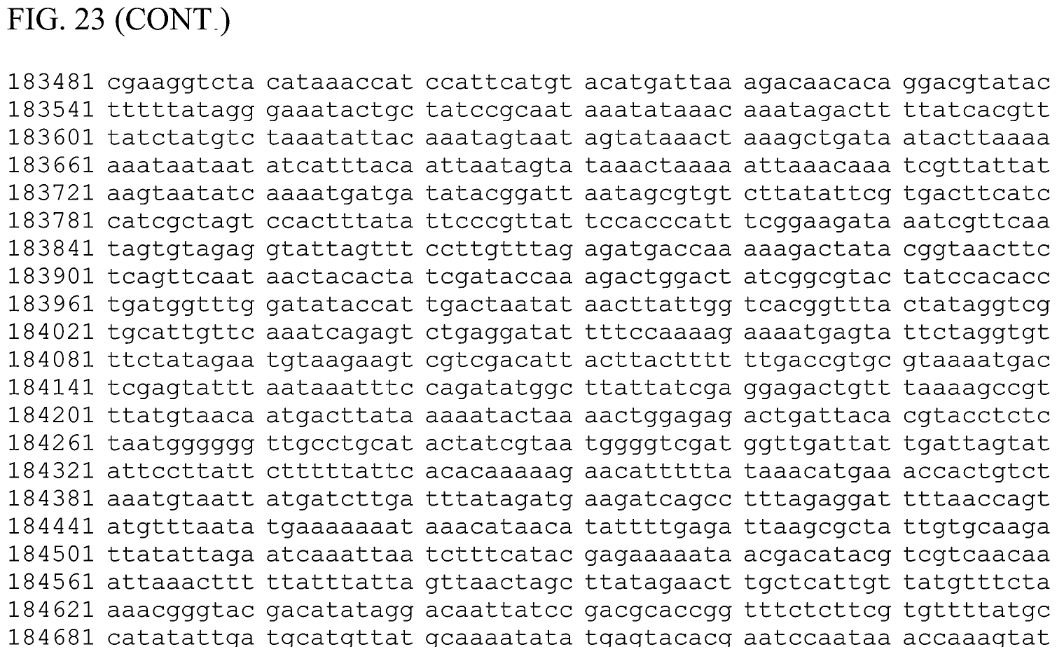

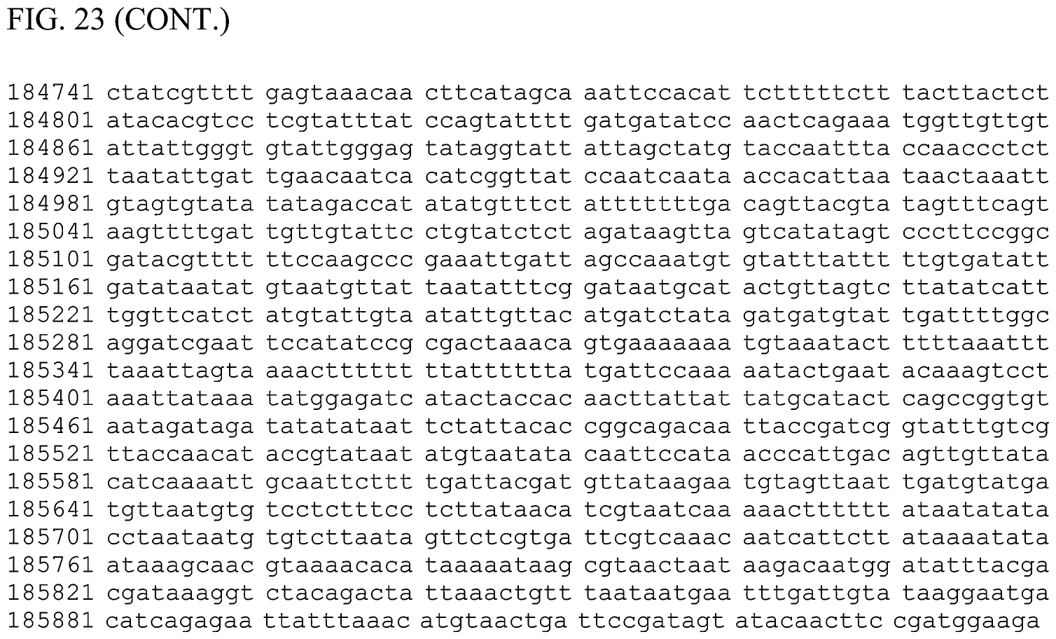

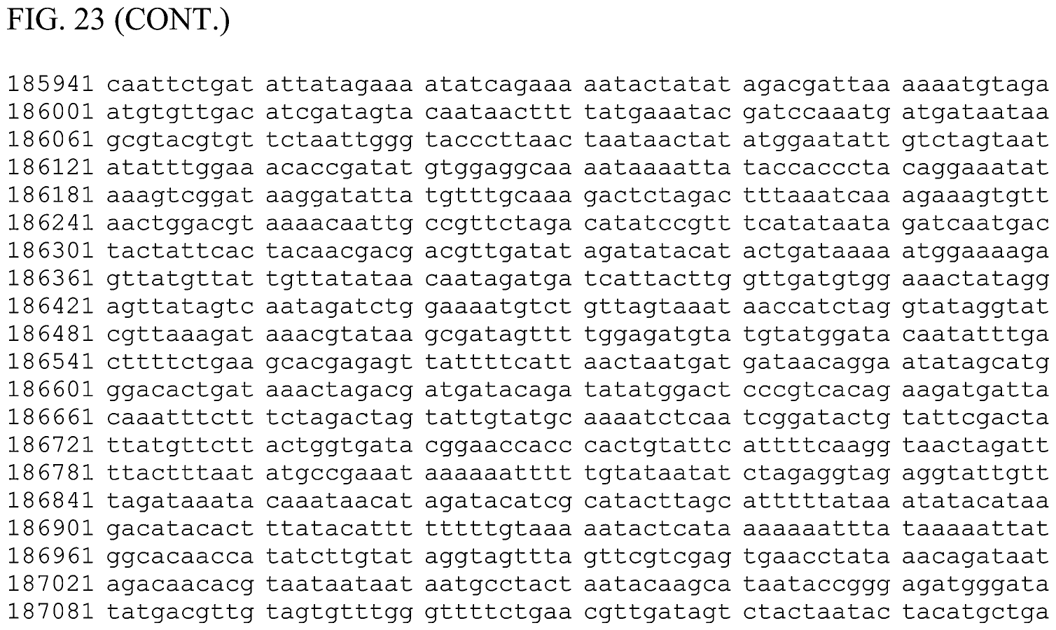

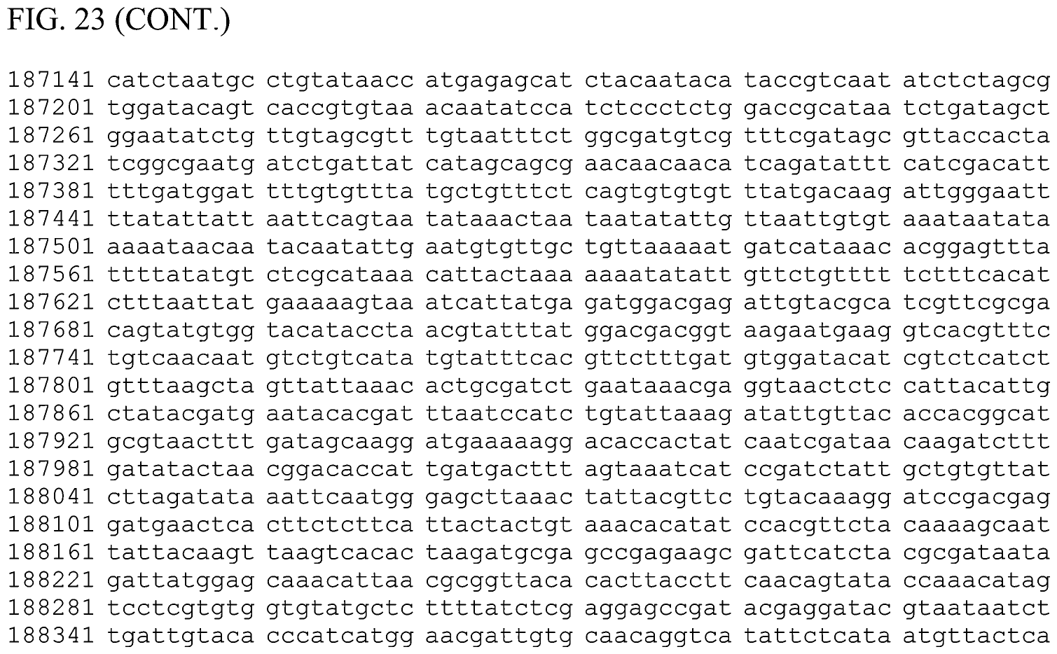

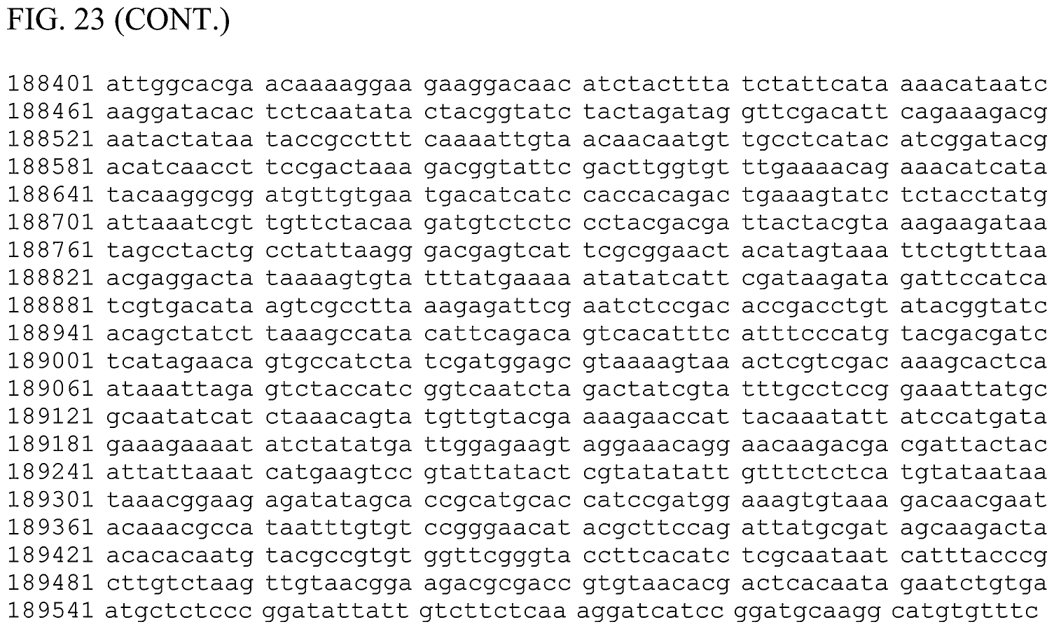

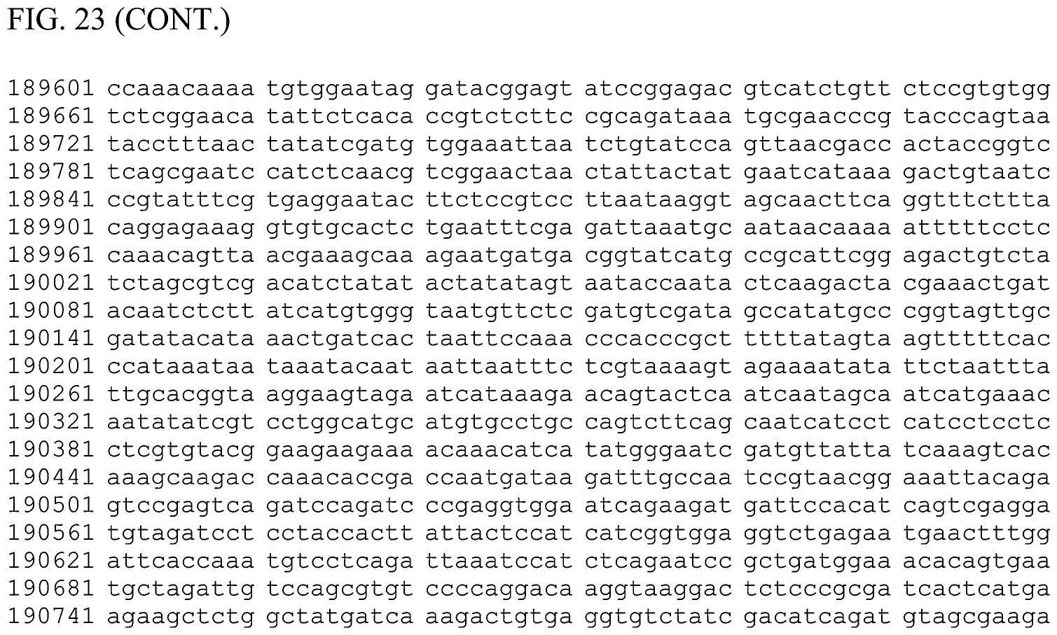

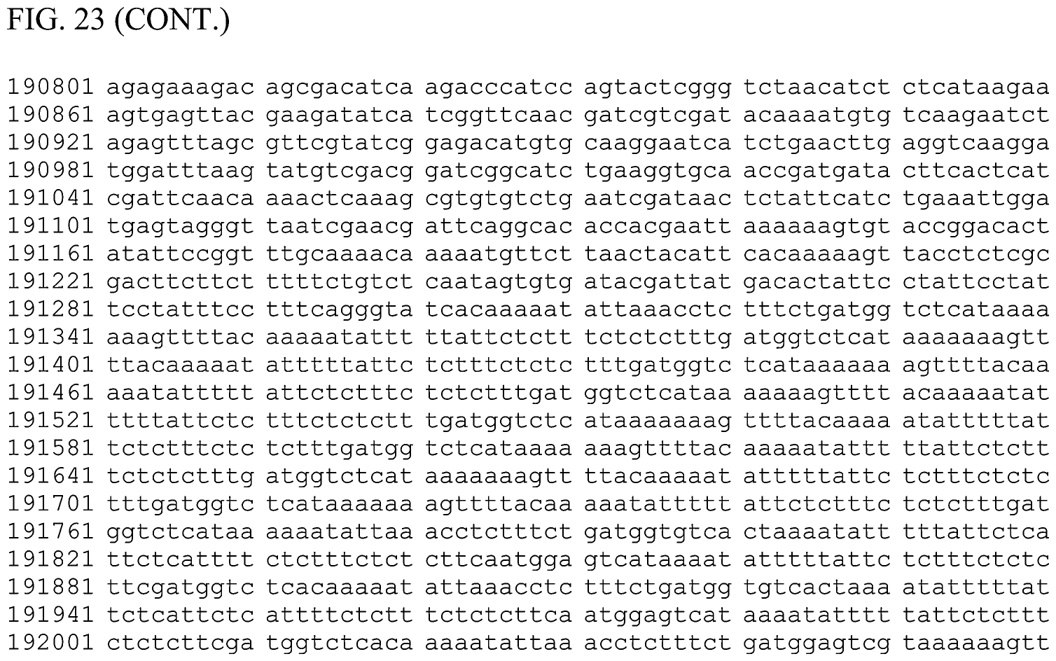

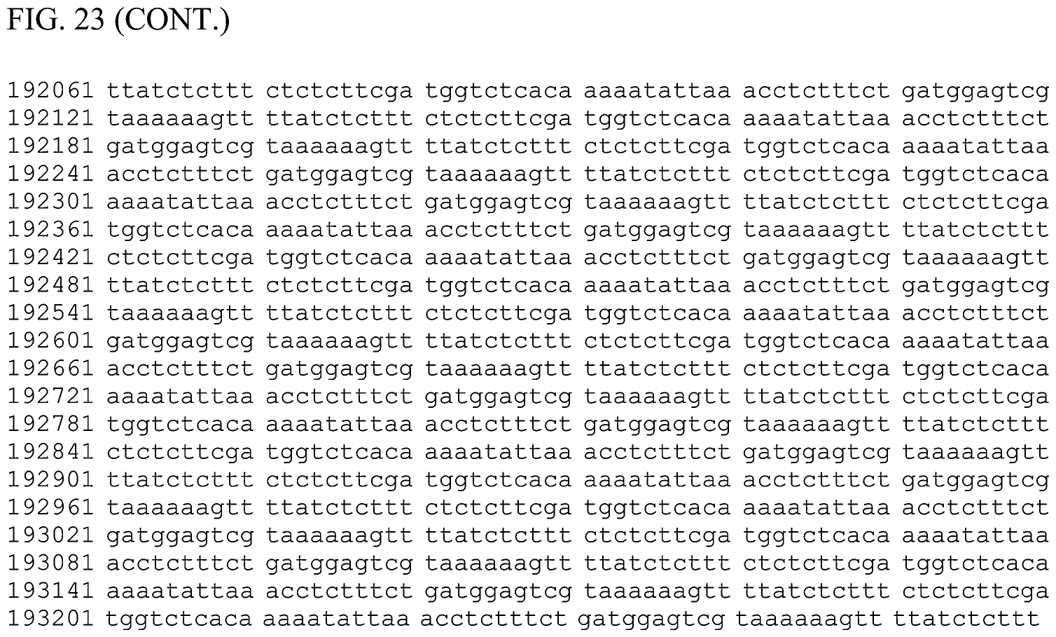

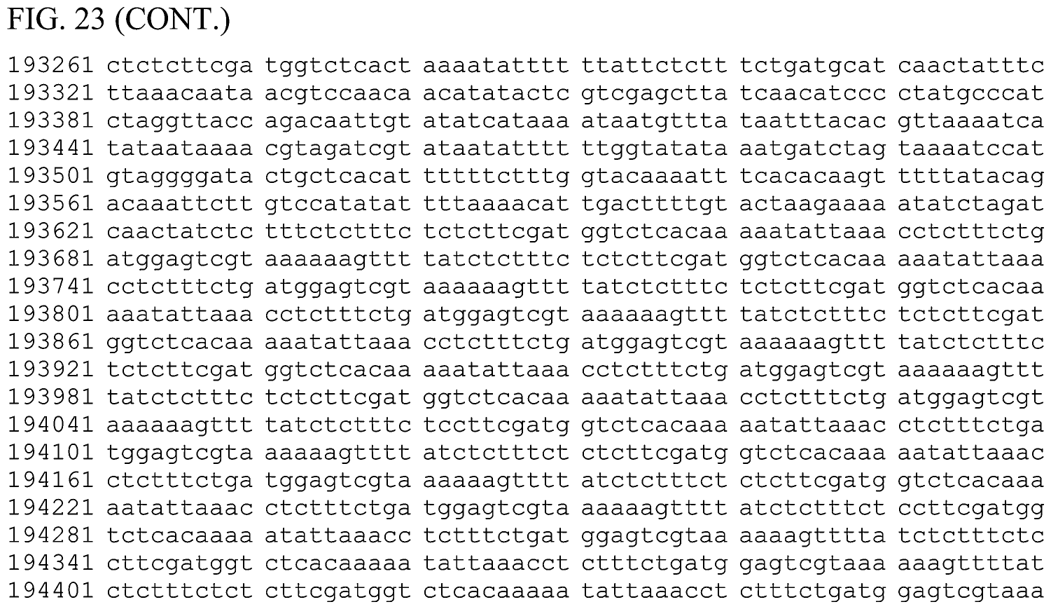

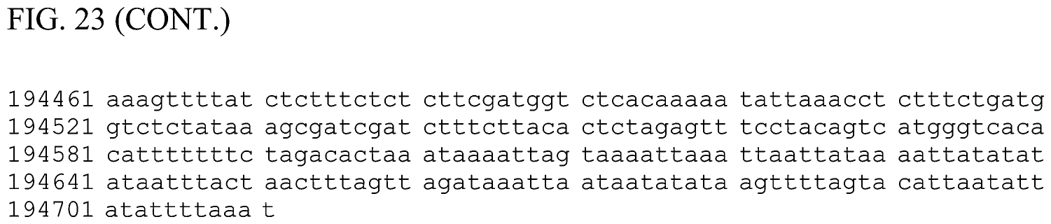

The MVA virus genome sequence (SEQ ID NO: 1) given by GenBank Accession No. is provided in FIG. 22. In some embodiments, engineered MVAΔC7L virus expressing OX40L is generated by inserting an expression construct such as those illustrated by SEQ ID NOs: 3 and 5 into the MVA genomic region that corresponds to the position of the TK locus (e.g., position 75,560 to 76,093 of SEQ ID NO: 1). Additionally or alternatively, in some embodiments engineered MVAΔC7L-OX40L is further modified to express hFlt3L by inserting an expression construct such as that which is illustrated by SEQ ID NO: 4 into the MVA genomic region that corresponds to the C7 locus (e.g., position 18,407 to 18,859 of SEQ ID NO: 1).

### MVAΔE3L

The disclosure of the present technology relates to an E3L mutant modified vaccinia Ankara (MVA) virus (i.e., MVAΔE3L; MVA virus comprising an E3L deletion; MVA virus genetically engineered to comprise a mutant E3L gene), or immunogenic compositions comprising the virus, in which the virus is engineered to express one or more specific genes of interest (SG), such as OX40L (MVAΔE3L-OX40L), and their use as a cancer immunotherapeutic. In some embodiments, the thymidine kinase (TK) gene of the MVA virus, through homologous recombination techniques, is engineered to contain a disruption comprising a heterologous nucleic acid sequence comprising one or more expression cassettes, which result in a TK knockout such that the TK gene is not expressed, expressed at levels so low as to have no effect, or the expressed protein is non-functional (e.g., is a null-mutation). The resulting MVAΔE3L-TK(−) virus is further engineered to comprise one or more expression cassettes that are flanked by a partial sequence of the TK gene (TK-L and TK-R) on either side. For example, in some embodiments, the nucleic acid sequence corresponding to the position of TK in the MVAΔE3L genome (e.g., position 75,798 to 75,868 of SEQ ID NO: 1) is replaced with a heterologous nucleic acid sequence comprising an open reading frame that encodes a specific gene of interest (SG), such as human OX40L, resulting in MVAΔE3L-TK(−)-OX40L. In some embodiments, the expression cassette comprises a single open reading frame that encodes a specific gene of interest (SG), such as OX40L using the vaccinia viral synthetic early and late promoter (PsE/L).

Although in certain embodiments described above, the transgene (e.g., OX40L) may be inserted into the TK locus, splitting the TK gene and obliterating it, other suitable integration loci can be selected. For example, MVA encodes several immune modulatory genes, including but not limited to C11, K3, F1, F2, F4, F6, F8, F9, F11, F14.5, J2, A46, E3L, B18R (WR200), E5R, K7R, C12L, B8R, B14R, N1L, C11R, K1L, C16, M1L, N2L, and WR199. Accordingly, in some embodiments, these genes can be deleted to potentially enhance immune activating properties of the virus, and allow insertion of transgenes.

In some embodiments, the recombinant MVAΔE3L-OX40L viruses described above are modified to express at least one other heterologous gene, such as any one or more of hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or include at least one other viral gene mutation or deletion, such as any one or more of the following deletions: C7; E3LΔ83N; B2R (ΔB2R), B19R (B18R; ΔWR200); E5R; K7R; C12L (IL18BP); B8R; B14R; N1L; C11R; K1L; M1L; N2L; and/or WR199. In other embodiments, no further heterologous genes are added other than those provided in the name of the virus herein (e.g., OX40L), and/or no further viral genes other than E3L or E3L and TK are disrupted or deleted.

In some embodiments, MVAΔE3L is engineered to express both OX40L and hFlt3L. In some embodiments, the recombinant virus is further modified at the E3 locus, through homologous recombination techniques, to contain a disruption comprising a heterologous nucleic acid sequence comprising one or more expression cassettes, which result in an E3 knockout such that the E3 gene is not expressed, expressed at levels so low as to have no effect, or the expressed protein is non-functional (e.g., is a null-mutation). In some embodiments, the expression cassette comprises a single open reading frame that encodes a specific gene of interest (SG), such as hFlt3L, resulting in MVAΔE3L-hFlt3L-TK(−)-OX40L. In some embodiments, the expression cassette encoding OX40L is inserted into the E3 locus while the expression cassette encoding hFlt3L is inserted into the TK locus.

Additionally or alternatively, in some embodiments, the heterologous nucleic acid sequence comprises an expression cassette comprising two or more open reading frames encoding two or more specific genes of interest, separated by a nucleotide sequence that encodes, in the 5′ to 3′ direction, a protease cleavage site (e.g., a furin cleavage site) and a 2A peptide (Pep2A) sequence.

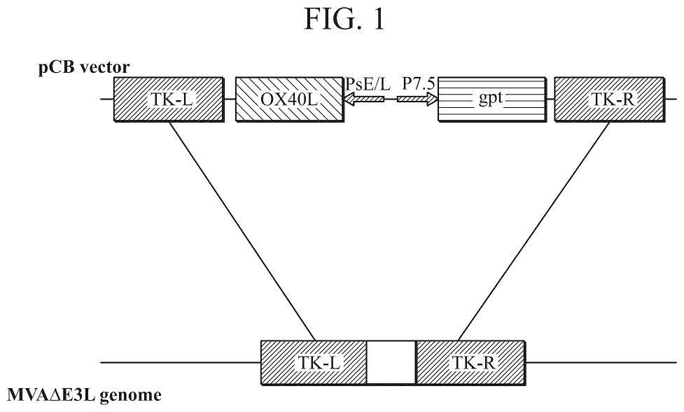

In some embodiments, the heterologous nucleotide sequence further comprises an additional expression cassette comprising an open reading frame that encodes a selectable marker operably linked to a promoter that is capable of directing expression of the selectable marker (see, e.g., FIG. 1). In some embodiments, the selectable marker is a xanthine-guanine phosphoribosyl transferase (gpt) gene. In some embodiments, the selectable marker is a green fluorescent protein (GFP) gene. In some embodiments, the selectable marker is an mCherry gene encoding a red fluorescent protein.

Non-limiting examples of OX40L expression construct open reading frames according to the present technology are shown above in Table 1.

### MVAΔE5R

The disclosure of the present technology relates to an E5R mutant modified vaccinia Ankara (MVA) virus (i.e., MVAΔE5R; MVA virus comprising an E5R deletion; MVA genetically engineered to comprise a mutant E5R gene), or immunogenic compositions comprising the virus, and their use as a cancer immunotherapeutic. In some embodiments, the E5R gene of the MVA virus, through homologous recombination techniques, is engineered to contain a disruption comprising a heterologous nucleic acid sequence comprising one or more expression cassettes, which result in an E5R knockout such that the E5R gene is not expressed, expressed at levels so low as to have no effect, or the expressed protein is non-functional (e.g., is a null-mutation). In some embodiments, the ΔE5R mutant includes a heterologous nucleic acid sequence in place of all or a majority of the E5R gene sequence. For example, in some embodiments, the nucleic acid sequence corresponding to the position of E5R in the MVA genome (e.g., position 38,432 to 39,385 of SEQ ID NO: 1) is replaced with a heterologous nucleic acid sequence comprising an open reading frame that encodes a specific gene of interest (SG), such as human OX40L, resulting in MVAΔE5R-OX40L. In some embodiments, the expression cassette comprises a single open reading frame that encodes hFlt3L, resulting in MVAΔE5R-hFlt3L.

In some embodiments, the MVAΔE5R virus is engineered to express one or more specific genes of interest (SG), such as a heterologous gene selected from any one or more of hOX40L, hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or include at least one other viral gene mutation or deletion, such as any one or more of the following deletions or mutations: C7 (AC7); E3L (ΔE3L); E3LΔ83N; B2R (ΔB2R), B19R (B18R; ΔWR200); E5R (ΔE5R); K7R; C12L (IL18BP); B8R; B14R; N1L; C11R; K1L; M1L; N2L; and/or WR199. In some embodiments, the MVAΔE5R virus is selected from MVAΔE3LΔE5R, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, MVAΔE3LΔE5R-hFlt3L-OX40LΔWR199-hIL-12ΔC11R, MVAΔE3LΔE5R-hFlt3L-OX40LΔWR199-hIL-12ΔC11R-hIL-15/IL-15Rα, or MVAΔE5R-hFlt3L-OX40L-ΔWR199.

In some embodiments, the thymidine kinase (TK) gene of the MVA virus, through homologous recombination techniques, is engineered to contain a disruption comprising a heterologous nucleic acid sequence comprising one or more expression cassettes, which result in a TK knockout such that the TK gene is not expressed, expressed at levels so low as to have no effect, or the expressed protein is non-functional (e.g., is a null-mutation). The resulting MVAΔE5R-TK(−) virus is further engineered to comprise one or more expression cassettes that are flanked by a partial sequence of the TK gene (TK-L and TK-R) on either side. For example, in some embodiments, the nucleic acid sequence corresponding to the position of TK in the MVAΔE5R genome (e.g., position 75,798 to 75,868 of SEQ ID NO: 1) is replaced with a heterologous nucleic acid sequence comprising an open reading frame that encodes a specific gene of interest (SG), such as human OX40L, resulting in MVAΔE5R-TK(−)-OX40L. In some embodiments, the expression cassette comprises a single open reading frame that encodes a specific gene of interest (SG), such as OX40L using the vaccinia viral synthetic early and late promoter (PsE/L).

Although in certain embodiments described above, the transgene (e.g., OX40L) may be inserted into the TK locus, splitting the TK gene and obliterating it, other suitable integration loci can be selected. For example, MVA encodes several immune modulatory genes, including but not limited to C11, K3, F1, F2, F4, F6, F8, F9, F11, F14.5, J2, A46, E3L, B18R (WR200), E5R, K7R, C12L, B8R, B14R, N1L, C11R, K1L, C16, M1L, N2L, and WR199. Accordingly, in some embodiments, these genes can be deleted to potentially enhance immune activating properties of the virus, and allow insertion of transgenes.

In other embodiments, no further heterologous genes are added other than those provided in the name of the virus herein (e.g., OX40L, hFlt3L), and/or no further viral genes other than E5R or E5R and TK are disrupted or deleted.

In some embodiments, MVAΔE5R is engineered to express both OX40L and hFlt3L. In some embodiments, the recombinant virus is further modified at the E5R locus, through homologous recombination techniques, to contain a disruption comprising a heterologous nucleic acid sequence comprising one or more expression cassettes, which result in an E5R knockout such that the E5R gene is not expressed, expressed at levels so low as to have no effect, or the expressed protein is non-functional (e.g., is a null-mutation). In some embodiments, the expression cassette comprises a single open reading frame that encodes a specific gene of interest (SG), such as hFlt3L, resulting in MVAΔE5R-hFlt3L-TK(−)-OX40L. In some embodiments, the expression cassette encoding OX40L is inserted into the E5R locus while the expression cassette encoding hFlt3L is inserted into the TK locus.

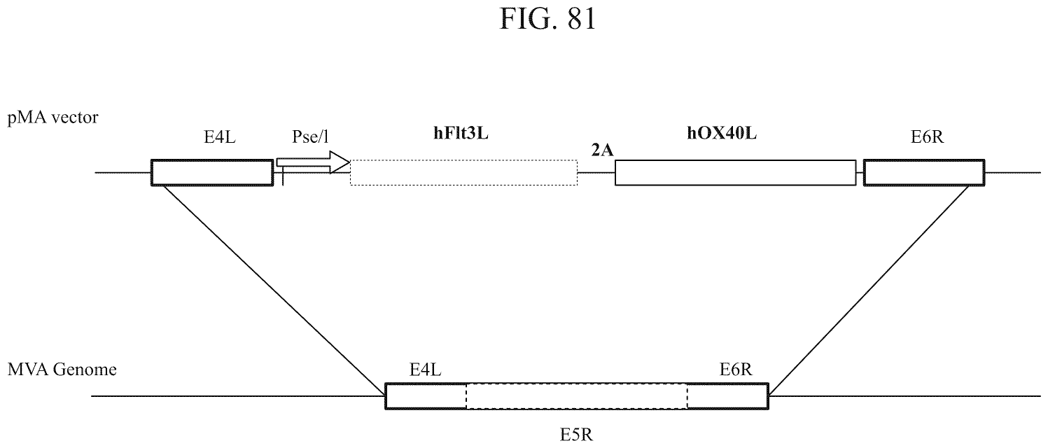

Additionally or alternatively, in some embodiments, the heterologous nucleic acid sequence comprises an expression cassette comprising two or more open reading frames encoding two or more specific genes of interest, separated by a nucleotide sequence that encodes, in the 5′ to 3′ direction, a protease cleavage site (e.g., a furin cleavage site) and a 2A peptide (Pep2A) sequence. For example, in some embodiments, MVAΔE5R encompasses a recombinant MVA in which all or a majority of the E5R gene sequence is replaced by a first specific gene of interest (e.g., hFtl3L) and a second specific gene of interest (e.g., OX40L), wherein the coding sequences of the first and second specific genes of interest are separated by a cassette including a furin cleavage site followed by a 2A peptide (Pep2A) sequence, thereby forming a recombinant virus such as MVAΔE5R-hFlt3L-OX40L (see, e.g., FIG. 81).

In some embodiments, the heterologous nucleotide sequence further comprises an additional expression cassette comprising an open reading frame that encodes a selectable marker operably linked to a promoter that is capable of directing expression of the selectable marker. In some embodiments, the selectable marker is a xanthine-guanine phosphoribosyl transferase (gpt) gene. In some embodiments, the selectable marker is a green fluorescent protein (GFP) gene. In some embodiments, the selectable marker is an mCherry gene encoding a red fluorescent protein.

Non-limiting examples of expression and deletion construct open reading frames according to the present technology are shown in (Table 2).

TABLE 2
Exemplary nucleotide sequences for the open reading frames of the
recombinant MVAΔE5R constructs of the present technology.

```
pUC57-MVA-ΔE5R-mCherry vector nucleic acid sequence
(SEQ ID NO: 22)
      1 TCGCGCGTTT CGGTGATGAC GGTGAAAACC TCTGACACAT GCAGCTCCCG GAGACGGTCA
    61 CAGCTTGTCT GTAAGCGGAT GCCGGGAGCA GACAAGCCCG TCAGGGCGCG TCAGCGGGTG
  121 TTGGCGGGTG TCGGGGCTGG CTTAACTATG CGGCATCAGA GCAGATTGTA CTGAGAGTGC
  181 ACCATATGCG GTGTGAAATA CCGCACAGAT GCGTAAGGAG AAAATACCGC ATCAGGCGCC
  241 ATTCGCCATT CAGGCTGCGC AACTGTTGGG AAGGGCGATC GGTGCGGGCC TCTTCGCTAT
  301 TACGCCAGCT GGCGAAAGGG GGATGTGCTG CAAGGCGATT AAGTTGGGTA ACGCCAGGGT
  361 TTTCCCAGTC ACGACGTTGT AAAACGACGG CCAGTGAATT CGAGCTCGGT ACCgagatag
  421 cgaaggaatt ctttttcggt gccgctagta cccttaatca tatcacatag tgttttatat
  481 tccaaatttg tggcaataga cggtttattt ctatacgata gtttgtttct ggaatccttt
  541 gagtattcta taccaatatt attctttgat tcgaatttag tttcttcgat attagatttt
  601 gtattaccta tattcttgat gtagtacttt gatgattttt ccatggccca ttctattaag
  661 tcttccaagt tggcatcatc cacatattgt gatagtaatt ctcggatatc agtagcggct
  721 accgccattg atgtttgttc attggatgag taactactaa tgtatacatt ttccatttat
  781 aacacttatg tattaacttt gttcatttat attttttcat tattatgttg atattaacaa
  841 aagtgaatat ataagcttga tcaggccttc actaattcca aacccacccg ctttttatag
  901 taagtttttc acccataaat aataaataca ataattaatt tctcgtaaaa gtagaaaata
  961 tattctaatt tattgcacgg taaggaagta gatcataact cgacatggtg agcaagggcg
1021 aggaggataa catggccatc atcaaggagt tcatgcgctt caaggtgcac atggagggct
1081 ccgtgaacgg ccacgagttc gagatcgagg gcgagggcga gggccgcccc tacgagggca
1141 cccagaccgc caagctgaag gtgaccaagg gtggccccct gcccttcgcc tgggacatcc
1201 tgtcccctca gttcatgtac ggctccaagg cctacgtgaa gcaccccgcc gacatccccg
1261 actacttgaa gctgtccttc cccgagggct tcaagtggga gcgcgtgatg aacttcgagg
1321 acggcggcgt ggtgaccgtg acccaggact cctccctgca ggacggcgag ttcatctaca
1381 aggtgaagct gcgcggcacc aacttcccct ccgacggccc cgtaatgcag aagaagacca
1441 tgggctggga ggcctcctcc gagcggatgt accccgagga cggcgccctg aagggcgaga
1501 tcaagcagag gctgaagctg aaggacggcg gccactacga cgctgaggtc aagaccacct
1561 acaaggccaa gaagcccgtg cagctgcccg gcgcctacaa cgtcaacatc aagttggaca
1621 tcacctccca caacgaggac tacaccatcg tggaacagta cgaacgcgcc gagggccgcc
1681 actccaccgg cggcatggac gagctGATCA CGAATTgtta acctgcattt catctttctc
1741 caatactaat tcaaattgtt aaattaataa tggatagtat aaatagttat tagtgataaa
1801 atagtaaaaa taattattag aataagagtg tagtatcata gataactctc ttctataaaa
1861 atggatttta ttcgtagaaa gtatcttata tacacagtag aaaataatat agatttttta
1921 aaggatgata cattaagtaa agtaaacaat tttaccctca atcatgtact agctctcaag
1981 tatctagtta gcaattttcc tcaacacgtt attactaagg atgtattagc taataccaat
2041 ttttttgttt tcatacatat ggtacgatgt tgtaaagtgt acgaagcggt tttacgacac
2101 gcatttgatg cacccacgtt gtacgttaaa gcattgacta agaattattG GATCCCGGGC
2161 CCGTCGACTG CAGAGGCCTG CATGCAAGCT TGGCGTAATC ATGGTCATAG CTGTTTCCTG
2221 TGTGAAATTG TTATCCGCTC ACAATTCCAC ACAACATACG AGCCGGAAGC ATAAAGTGTA
2281 AAGCCTGGGG TGCCTAATGA GTGAGCTAAC TCACATTAAT TGCGTTGCGC TCACTGCCCG
2341 CTTTCCAGTC GGGAAACCTG TCGTGCCAGC TGCATTAATG AATCGGCCAA CGCGCGGGGA
2401 GAGGCGGTTT GCGTATTGGG CGCTCTTCCG CTTCCTCGCT CACTGACTCG CTGCGCTCGG
2461 TCGTTCGGCT GCGGCGAGCG GTATCAGCTC ACTCAAAGGC GGTAATACGG TTATCCACAG
2521 AATCAGGGGA TAACGCAGGA AAGAACATGT GAGCAAAAGG CCAGCAAAAG GCCAGGAACC
2581 GTAAAAAGGC CGCGTTGCTG GCGTTTTTCC ATAGGCTCCG CCCCCCTGAC GAGCATCACA
2641 AAAATCGACG CTCAAGTCAG AGGTGGCGAA ACCCGACAGG ACTATAAAGA TACCAGGCGT
2701 TTCCCCCTGG AAGCTCCCTC GTGCGCTCTC CTGTTCCGAC CCTGCCGCTT ACCGGATACC
2761 TGTCCGCCTT TCTCCCTTCG GGAAGCGTGG CGCTTTCTCA TAGCTCACGC TGTAGGTATC
2821 TCAGTTCGGT GTAGGTCGTT CGCTCCAAGC TGGGCTGTGT GCACGAACCC CCCGTTCAGC
2881 CCGACCGCTG CGCCTTATCC GGTAACTATC GTCTTGAGTC CAACCCGGTA AGACACGACT
2941 TATCGCCACT GGCAGCAGCC ACTGGTAACA GGATTAGCAG AGCGAGGTAT GTAGGCGGTG
3001 CTACAGAGTT CTTGAAGTGG TGGCCTAACT ACGGCTACAC TAGAAGAACA GTATTTGGTA
3061 TCTGCGCTCT GCTGAAGCCA GTTACCTTCG GAAAAAGAGT TGGTAGCTCT TGATCCGGCA
3121 AACAAACCAC CGCTGGTAGC GGTGGTTTTT TTGTTTGCAA GCAGCAGATT ACGCGCAGAA
3181 AAAAAGGATC TCAAGAAGAT CCTTTGATCT TTTCTACGGG GTCTGACGCT CAGTGGAACG
3241 AAAACTCACG TTAAGGGATT TTGGTCATGA GATTATCAAA AAGGATCTTC ACCTAGATCC
3301 TTTTAAATTA AAAATGAAGT TTTAAATCAA TCTAAAGTAT ATATGAGTAA ACTTGGTCTG
3361 ACAGTTACCA ATGCTTAATC AGTGAGGCAC CTATCTCAGC GATCTGTCTA TTTCGTTCAT
3421 CCATAGTTGC CTGACTCCCC GTCGTGTAGA TAACTACGAT ACGGGAGGGC TTACCATCTG
3481 GCCCCAGTGC TGCAATGATA CCGCGAGACC CACGCTCACC GGCTCCAGAT TTATCAGCAA
3541 TAAACCAGCC AGCCGGAAGG GCCGAGCGCA GAAGTGGTCC TGCAACTTTA TCCGCCTCCA
3601 TCCAGTCTAT TAATTGTTGC CGGGAAGCTA GAGTAAGTAG TTCGCCAGTT AATAGTTTGC
3661 GCAACGTTGT TGCCATTGCT ACAGGCATCG TGGTGTCACG CTCGTCGTTT GGTATGGCTT
3721 CATTCAGCTC CGGTTCCCAA CGATCAAGGC GAGTTACATG ATCCCCCATG TTGTGCAAAA
3781 AAGCGGTTAG CTCCTTCGGT CCTCCGATCG TTGTCAGAAG TAAGTTGGCC GCAGTGTTAT
3841 CACTCATGGT TATGGCAGCA CTGCATAATT CTCTTACTGT CATGCCATCC GTAAGATGCT
3901 TTTCTGTGAC TGGTGAGTAC TCAACCAAGT CATTCTGAGA ATAGTGTATG CGGCGACCGA
3961 GTTGCTCTTG CCCGGCGTCA ATACGGGATA ATACCGCGCC ACATAGCAGA ACTTTAAAAG
4021 TGCTCATCAT TGGAAAACGT TCTTCGGGGC GAAAACTCTC AAGGATCTTA CCGCTGTTGA
4081 GATCCAGTTC GATGTAACCC ACTCGTGCAC CCAACTGATC TTCAGCATCT TTTACTTTCA
4141 CCAGCGTTTC TGGGTGAGCA AAAACAGGAA GGCAAAATGC CGCAAAAAAG GGAATAAGGG
4201 CGACACGGAA ATGTTGAATA CTCATACTCT TCCTTTTTCA ATATTATTGA AGCATTTATC
4261 AGGGTTATTG TCTCATGAGC GGATACATAT TTGAATGTAT TTAGAAAAAT AAACAAATAG
4321 GGGTTCCGCG CACATTTCCC CGAAAAGTGC CACCTGACGT CTAAGAAACC ATTATTATCA
4381 TGACATTAAC CTATAAAAAT AGGCGTATCA CGAGGCCCTT TCGTC
pMA-MVA-ΔE5R-hFlt3L-hOX40L vector nucleic acid sequence
(SEQ ID NO: 23)
      1 CTAAATTGTA AGCGTTAATA TTTTGTTAAA ATTCGCGTTA AATTTTTGTT AAATCAGCTC
    61 ATTTTTTAAC CAATAGGCCG AAATCGGCAA AATCCCTTAT AAATCAAAAG AATAGACCGA
  121 GATAGGGTTG AGTGGCCGCT ACAGGGCGCT CCCATTCGCC ATTCAGGCTG CGCAACTGTT
  181 GGGAAGGGCG TTTCGGTGCG GGCCTCTTCG CTATTACGCC AGCTGGCGAA AGGGGGATGT
  241 GCTGCAAGGC GATTAAGTTG GGTAACGCCA GGGTTTTCCC AGTCACGACG TTGTAAAACG
  301 ACGGCCAGTG AGCGCGACGT AATACGACTC ACTATAGGGC GAATTGGCGG AAGGCCGTCA
  361 AGGCCGCATT GGAGGTTCGT CAGCGGCTCT AGTTTGAATC ATCATCGGCG TAGTATTCCT
  421 ACTTTTACAG TTAGGACACG GTGTATTGTA TTTCTCGTCG AGAACGTTAA AATAATCGTT
  481 GTAACTCACA TCCTTTATTT TATCTATATT GTATTCTACT CCTTTCTTAA TGCATTTTAT
  541 ACCGAATAAG AGATAGCGAA GGAATTCTTT TTCGGTGCCG CTAGTACCCT TAATCATATC
  601 ACATAGTGTT TTATATTCCA AATTTGTGGC AATAGACGGT TTATTTCTAT ACGATAGTTT
  661 GTTTCTGGAA TCCTTTGAGT ATTCTATACC AATATTATTC TTTGATTCGA ATTTAGTTTC
  721 TTCGATATTA GATTTTGTAT TACCTATATT CTTGATGTAG TACTTTGATG ATTTTTCCAT
  781 GGCCCATTCT ATTAAGTCTT CCAAGTTGGC ATCATCCACA TATTGTGATA GTAATTCTCG
  841 GATATCAGTA GCGGCTACCG CCATTGATGT TTGTTCATTG GATGAGTAAC TACTAATGTA
  901 TACATTTTCC ATTTATAACA CTTATGTATT AACTTTGTTC ATTTATATTT TTTCATTATT
  961 ATGTTGATAT TAACAAAAGT GAATATATGG ATCCAAAAAT TGAAATTTTA TTTTTTTTTT
1021 TTGGAATATA AATAAGCTCG AAGTCGACGA ATTCATGACA GTACTAGCTC CAGCTTGGTC
1081 CCCGACAACA TACCTTCTAC TACTACTATT GCTATCCTCC GGACTATCTG GAACCCAGGA
1141 TTGCTCTTTT CAGCACTCTC CGATCTCGTC TGATTTCGCG GTTAAGATCA GAGAGCTATC
1201 CGACTACTTG CTACAGGATT ACCCAGTAAC CGTCGCGTCC AACCTACAAG ATGAAGAACT
1261 ATGTGGTGGA CTTTGGAGAC TAGTCCTAGC GCAAAGATGG ATGGAAAGAC TTAAGACCGT
1321 AGCGGGATCT AAGATGCAGG GACTACTAGA AAGAGTCAAC ACCGAGATCC ACTTCGTCAC
1381 AAAGTGTGCG TTTCAACCAC CACCGTCCTG TCTAAGATTC GTCCAGACAA ACATCTCCAG
1441 ACTACTACAA GAAACCTCCG AGCAGCTAGT AGCGCTAAAA CCGTGGATCA CAAGACAGAA
1501 CTTCTCGAGA TGTCTAGAGC TACAGTGTCA GCCGGATTCT TCTACATTAC CACCACCATG
1561 GTCACCAAGA CCACTAGAAG CTACAGCTCC AACTGCTCCA CAACCACCAT TGCTACTTTT
1621 GCTATTGCTA CCCGTCGGAT TGCTACTATT AGCTGCTGCT TGGTGTCTAC ACTGGCAGAG
1681 AACTAGAAGA AGAACTCCAA GACCGGGAGA ACAAGTACCA CCAGTACCAT CTCCACAGGA
1741 CCTACTACTA GTCGAGCACA GAAGAAGAAG AAGATCGGGA GCGACCAACT TCTCGCTATT
1801 GAAACAAGCG GGAGATGTCG AAGAAAATCC GGGACCAATG GAAAGAGTAC AGCCGCTAGA
1861 AGAAAACGTA GGAAATGCGG CTAGACCGAG ATTCGAGAGA AACAAGCTAC TATTGGTCGC
1921 GTCCGTCATC CAAGGACTAG GATTGCTATT GTGCTTCACC TACATCTGCC TACACTTCTC
1981 CGCGCTACAA GTCTCTCATA GATACCCGAG AATCCAGTCC ATCAAGGTCC AGTTCACCGA
2041 GTACAAGAAA GAGAAGGGAT TCATCCTAAC CTCGCAGAAA GAGGACGAGA TCATGAAGGT
2101 CCAGAACAAC TCCGTCATCA TCAACTGCGA CGGATTCTAC CTAATCTCCC TAAAGGGATA
2161 CTTCTCCCAA GAAGTCAACA TCTCCTTGCA CTACCAGAAG GATGAGGAAC CGCTATTCCA
2221 GCTAAAGAAA GTCAGATCCG TCAACTCCCT AATGGTCGCC TCTCTAACGT ACAAGGACAA
2281 GGTCTACCTA AACGTCACCA CCGACAACAC ATCCCTAGAT GATTTCCACG TAAACGGTGG
2341 AGAGCTAATC CTAATCCATC AGAACCCGGG AGAGTTCTGT GTATTATAAG TTAACAGGCC
2401 TGCATTTCAT CTTTCTCCAA TACTAATTCA AATTGTTAAA TTAATAATGG ATAGTATAAA
2461 TAGTTATTAG TGATAAAATA GTAAAAATAA TTATTAGAAT AAGAGTGTAG TATCATAGAT
2521 AACTCTCTTC TATAAAAATG GATTTTATTC GTAGAAAGTA TCTTATATAC ACAGTAGAAA
2581 ATAATATAGA TTTTTTAAAG GATGATACAT TAAGTAAAGT AAACAATTTT ACCCTCAATC
2641 ATGTACTAGC TCTCAAGTAT CTAGTTAGCA ATTTTCCTCA ACACGTTATT ACTAAGGATG
2701 TATTAGCTAA TACCAATTTT TTTGTTTTCA TACATATGGT ACGATGTTGT AAAGTGTACG
2761 AAGCGGTTTT ACGACACGCA TTTGATGCAC CCACGTTGTA CGTTAAAGCA TTGACTAAGA
2821 ATTATTTATC GTTTAGTAAC GCAATACAAT CGTACAAGGA AACCGTGCAT AAACTAACAC
2881 AAGATGAAAA ATTTTTAGAG GTTGCCGAAT ACATGGACGA ATTAGGAGAA CTTATAGGCG
2941 TAAATTATGA CTTAGTTCTT AATCCATTAT TTCACGGAGG GGAACCCATC AAAGATATGG
3001 AAATCACTGG GCCTCATGGG CCTTCCGCTC ACTGCCCGCT TTCCAGTCGG GAAACCTGTC
3061 GTGCCAGCTG CATTAACATG GTCATAGCTG TTTCCTTGCG TATTGGGCGC TCTCCGCTTC
3121 CTCGCTCACT GACTCGCTGC GCTCGGTCGT TCGGGTAAAG CCTGGGGTGC CTAATGAGCA
3181 AAAGGCCAGC AAAAGGCCAG GAACCGTAAA AAGGCCGCGT TGCTGGCGTT TTTCCATAGG
3241 CTCCGCCCCC CTGACGAGCA TCACAAAAAT CGACGCTCAA GTCAGAGGTG GCGAAACCCG
3301 ACAGGACTAT AAAGATACCA GGCGTTTCCC CCTGGAAGCT CCCTCGTGCG CTCTCCTGTT
3361 CCGACCCTGC CGCTTACCGG ATACCTGTCC GCCTTTCTCC CTTCGGGAAG CGTGGCGCTT
3421 TCTCATAGCT CACGCTGTAG GTATCTCAGT TCGGTGTAGG TCGTTCGCTC CAAGCTGGGC
3481 TGTGTGCACG AACCCCCCGT TCAGCCCGAC CGCTGCGCCT TATCCGGTAA CTATCGTCTT
3541 GAGTCCAACC CGGTAAGACA CGACTTATCG CCACTGGCAG CAGCCACTGG TAACAGGATT
3601 AGCAGAGCGA GGTATGTAGG CGGTGCTACA GAGTTCTTGA AGTGGTGGCC TAACTACGGC
3661 TACACTAGAA GAACAGTATT TGGTATCTGC GCTCTGCTGA AGCCAGTTAC CTTCGGAAAA
3721 AGAGTTGGTA GCTCTTGATC CGGCAAACAA ACCACCGCTG GTAGCGGTGG TTTTTTTGTT
3781 TGCAAGCAGC AGATTACGCG CAGAAAAAAA GGATCTCAAG AAGATCCTTT GATCTTTTCT
3841 ACGGGGTCTG ACGCTCAGTG GAACGAAAAC TCACGTTAAG GGATTTTGGT CATGAGATTA
3901 TCAAAAAGGA TCTTCACCTA GATCCTTTTA AATTAAAAAT GAAGTTTTAA ATCAATCTAA
3961 AGTATATATG AGTAAACTTG GTCTGACAGT TACCAATGCT TAATCAGTGA GGCACCTATC
4021 TCAGCGATCT GTCTATTTCG TTCATCCATA GTTGCCTGAC TCCCCGTCGT GTAGATAACT
4081 ACGATACGGG AGGGCTTACC ATCTGGCCCC AGTGCTGCAA TGATACCGCG AGAACCACGC
4141 TCACCGGCTC CAGATTTATC AGCAATAAAC CAGCCAGCCG GAAGGGCCGA GCGCAGAAGT
4201 GGTCCTGCAA CTTTATCCGC CTCCATCCAG TCTATTAATT GTTGCCGGGA AGCTAGAGTA
4261 AGTAGTTCGC CAGTTAATAG TTTGCGCAAC GTTGTTGCCA TTGCTACAGG CATCGTGGTG
4321 TCACGCTCGT CGTTTGGTAT GGCTTCATTC AGCTCCGGTT CCCAACGATC AAGGCGAGTT
4381 ACATGATCCC CCATGTTGTG CAAAAAAGCG GTTAGCTCCT TCGGTCCTCC GATCGTTGTC
4441 AGAAGTAAGT TGGCCGCAGT GTTATCACTC ATGGTTATGG CAGCACTGCA TAATTCTCTT
4501 ACTGTCATGC CATCCGTAAG ATGCTTTTCT GTGACTGGTG AGTACTCAAC CAAGTCATTC
4561 TGAGAATAGT GTATGCGGCG ACCGAGTTGC TCTTGCCCGG CGTCAATACG GGATAATACC
4621 GCGCCACATA GCAGAACTTT AAAAGTGCTC ATCATTGGAA AACGTTCTTC GGGGCGAAAA
4681 CTCTCAAGGA TCTTACCGCT GTTGAGATCC AGTTCGATGT AACCCACTCG TGCACCCAAC
4741 TGATCTTCAG CATCTTTTAC TTTCACCAGC GTTTCTGGGT GAGCAAAAAC AGGAAGGCAA
4801 AATGCCGCAA AAAAGGGAAT AAGGGCGACA CGGAAATGTT GAATACTCAT ACTCTTCCTT
4861 TTTCAATATT ATTGAAGCAT TTATCAGGGT TATTGTCTCA TGAGCGGATA CATATTTGAA
4921 TGTATTTAGA AAAATAAACA AATAGGGGTT CCGCGCACAT TTCCCCGAAA AGTGCCAC
pMA-MVAΔE5R-hFlt3L-mOX40L vector nucleic acid sequence
(SEQ ID NO: 24)
      1 CTAAATTGTA AGCGTTAATA TTTTGTTAAA ATTCGCGTTA AATTTTTGTT AAATCAGCTC
    61 ATTTTTTAAC CAATAGGCCG AAATCGGCAA AATCCCTTAT AAATCAAAAG AATAGACCGA
  121 GATAGGGTTG AGTGGCCGCT ACAGGGCGCT CCCATTCGCC ATTCAGGCTG CGCAACTGTT
  181 GGGAAGGGCG TTTCGGTGCG GGCCTCTTCG CTATTACGCC AGCTGGCGAA AGGGGGATGT
  241 GCTGCAAGGC GATTAAGTTG GGTAACGCCA GGGTTTTCCC AGTCACGACG TTGTAAAACG
  301 ACGGCCAGTG AGCGCGACGT AATACGACTC ACTATAGGGC GAATTGGCGG AAGGCCGTCA
  361 AGGCCGCATT GGAGGTTCGT CAGCGGCTCT AGTTTGAATC ATCATCGGCG TAGTATTCCT
  421 ACTTTTACAG TTAGGACACG GTGTATTGTA TTTCTCGTCG AGAACGTTAA AATAATCGTT
  481 GTAACTCACA TCCTTTATTT TATCTATATT GTATTCTACT CCTTTCTTAA TGCATTTTAT
  541 ACCGAATAAG AGATAGCGAA GGAATTCTTT TTCGGTGCCG CTAGTACCCT TAATCATATC
  601 ACATAGTGTT TTATATTCCA AATTTGTGGC AATAGACGGT TTATTTCTAT ACGATAGTTT
  661 GTTTCTGGAA TCCTTTGAGT ATTCTATACC AATATTATTC TTTGATTCGA ATTTAGTTTC
  721 TTCGATATTA GATTTTGTAT TACCTATATT CTTGATGTAG TACTTTGATG ATTTTTCCAT
  781 GGCCCATTCT ATTAAGTCTT CCAAGTTGGC ATCATCCACA TATTGTGATA GTAATTCTCG
  841 GATATCAGTA GCGGCTACCG CCATTGATGT TTGTTCATTG GATGAGTAAC TACTAATGTA
  901 TACATTTTCC ATTTATAACA CTTATGTATT AACTTTGTTC ATTTATATTT TTTCATTATT
  961 ATGTTGATAT TAACAAAAGT GAATATATGG ATCCAAAAAT TGAAATTTTA TTTTTTTTTT
1021 TTGGAATATA AATAAGCTCG AAGTCGACGA ATTCATGACA GTACTAGCTC CAGCTTGGTC
1081 CCCGACAACA TACCTTCTAC TACTACTATT GCTATCCTCC GGACTATCTG GAACCCAGGA
1141 TTGCTCTTTT CAGCACTCTC CGATCTCGTC TGATTTCGCG GTTAAGATCA GAGAGCTATC
1201 CGACTACTTG CTACAGGATT ACCCAGTAAC CGTCGCGTCC AACCTACAAG ATGAAGAACT
1261 ATGTGGTGGA CTTTGGAGAC TAGTCCTAGC GCAAAGATGG ATGGAAAGAC TTAAGACCGT
1321 AGCGGGATCT AAGATGCAGG GACTACTAGA AAGAGTCAAC ACCGAGATCC ACTTCGTCAC
1381 AAAGTGTGCG TTTCAACCAC CACCGTCCTG TCTAAGATTC GTCCAGACAA ACATCTCCAG
1441 ACTACTACAA GAAACCTCCG AGCAGCTAGT AGCGCTAAAA CCGTGGATCA CAAGACAGAA
1501 CTTCTCGAGA TGTCTAGAGC TACAGTGTCA GCCGGATTCT TCTACATTAC CACCACCATG
1561 GTCACCAAGA CCACTAGAAG CTACAGCTCC AACTGCTCCA CAACCACCAT TGCTACTTTT
1621 GCTATTGCTA CCCGTCGGAT TGCTACTATT AGCTGCTGCT TGGTGTCTAC ACTGGCAGAG
1681 AACTAGAAGA AGAACTCCAA GACCGGGAGA ACAAGTACCA CCAGTACCAT CTCCACAGGA
1741 CCTACTACTA GTCGAGCACA GAAGAAGAAG AAGATCGGGA GCGACCAACT TCTCGCTATT
1801 GAAACAAGCG GGAGATGTCG AAGAAAATCC GGGACCAATG GAGGGCGAGG GGGTCCAGCC
1861 TCTGGACGAG AACCTCGAAA ACGGGTCTCG CCCTCGCTTT AAATGGAAGA AGACTCTTAG
1921 GCTCGTTGTA AGCGGCATCA AGGGGGCCGG TATGTTGCTG TGCTTCATAT ATGTGTGTTT
1981 GCAACTTAGC TCTTCACCTG CAAAAGACCC CCCCATACAA CGCCTTCGGG GGGCTGTGAC
2041 CCGCTGTGAA GATGGTCAAT TGTTTATTTC TTCTTACAAG AACGAGTATC AGACGATGGA
2101 AGTCCAGAAT AACTCCGTAG TGATTAAGTG TGACGGACTG TACATCATCT ACTTGAAAGG
2161 ATCTTTTTTC CAGGAGGTCA AAATTGACCT CCACTTCAGG GAGGATCACA ACCCTATCTC
2221 AATCCCTATG TTGAACGACG GCAGAAGAAT CGTCTTTACT GTAGTCGCTT CACTGGCCTT
2281 CAAGGATAAG GTGTACTTGA CCGTAAACGC TCCTGATACC TTGTGCGAGC ATTTGCAAAT
2341 CAACGATGGA GAACTTATCG TTGTCCAACT CACACCAGGT TACTGTGCTC CTGAGGGCAG
2401 TTATCACAGT ACAGTGAACC AAGTCCCACT GTGAGTTAAC AGGCCTGCAT TTCATCTTTC
2461 TCCAATACTA ATTCAAATTG TTAAATTAAT AATGGATAGT ATAAATAGTT ATTAGTGATA
2521 AAATAGTAAA AATAATTATT AGAATAAGAG TGTAGTATCA TAGATAACTC TCTTCTATAA
2581 AAATGGATTT TATTCGTAGA AAGTATCTTA TATACACAGT AGAAAATAAT ATAGATTTTT
2641 TAAAGGATGA TACATTAAGT AAAGTAAACA ATTTTACCCT CAATCATGTA CTAGCTCTCA
2701 AGTATCTAGT TAGCAATTTT CCTCAACACG TTATTACTAA GGATGTATTA GCTAATACCA
2761 ATTTTTTTGT TTTCATACAT ATGGTACGAT GTTGTAAAGT GTACGAAGCG GTTTTACGAC
2821 ACGCATTTGA TGCACCCACG TTGTACGTTA AAGCATTGAC TAAGAATTAT TTATCGTTTA
2881 GTAACGCAAT ACAATCGTAC AAGGAAACCG TGCATAAACT AACACAAGAT GAAAAATTTT
2941 TAGAGGTTGC CGAATACATG GACGAATTAG GAGAACTTAT AGGCGTAAAT TATGACTTAG
3001 TTCTTAATCC ATTATTTCAC GGAGGGGAAC CCATCAAAGA TATGGAAATC ACTGGGCCTC
3061 ATGGGCCTTC CGCTCACTGC CCGCTTTCCA GTCGGGAAAC CTGTCGTGCC AGCTGCATTA
3121 ACATGGTCAT AGCTGTTTCC TTGCGTATTG GGCGCTCTCC GCTTCCTCGC TCACTGACTC
3181 GCTGCGCTCG GTCGTTCGGG TAAAGCCTGG GGTGCCTAAT GAGCAAAAGG CCAGCAAAAG
3241 GCCAGGAACC GTAAAAAGGC CGCGTTGCTG GCGTTTTTCC ATAGGCTCCG CCCCCCTGAC
3301 GAGCATCACA AAAATCGACG CTCAAGTCAG AGGTGGCGAA ACCCGACAGG ACTATAAAGA
3361 TACCAGGCGT TTCCCCCTGG AAGCTCCCTC GTGCGCTCTC CTGTTCCGAC CCTGCCGCTT
3421 ACCGGATACC TGTCCGCCTT TCTCCCTTCG GGAAGCGTGG CGCTTTCTCA TAGCTCACGC
3481 TGTAGGTATC TCAGTTCGGT GTAGGTCGTT CGCTCCAAGC TGGGCTGTGT GCACGAACCC
3541 CCCGTTCAGC CCGACCGCTG CGCCTTATCC GGTAACTATC GTCTTGAGTC CAACCCGGTA
3601 AGACACGACT TATCGCCACT GGCAGCAGCC ACTGGTAACA GGATTAGCAG AGCGAGGTAT
3661 GTAGGCGGTG CTACAGAGTT CTTGAAGTGG TGGCCTAACT ACGGCTACAC TAGAAGAACA
3721 GTATTTGGTA TCTGCGCTCT GCTGAAGCCA GTTACCTTCG GAAAAAGAGT TGGTAGCTCT
3781 TGATCCGGCA AACAAACCAC CGCTGGTAGC GGTGGTTTTT TTGTTTGCAA GCAGCAGATT
3841 ACGCGCAGAA AAAAAGGATC TCAAGAAGAT CCTTTGATCT TTTCTACGGG GTCTGACGCT
3901 CAGTGGAACG AAAACTCACG TTAAGGGATT TTGGTCATGA GATTATCAAA AAGGATCTTC
3961 ACCTAGATCC TTTTAAATTA AAAATGAAGT TTTAAATCAA TCTAAAGTAT ATATGAGTAA
4021 ACTTGGTCTG ACAGTTACCA ATGCTTAATC AGTGAGGCAC CTATCTCAGC GATCTGTCTA
4081 TTTCGTTCAT CCATAGTTGC CTGACTCCCC GTCGTGTAGA TAACTACGAT ACGGGAGGGC
4141 TTACCATCTG GCCCCAGTGC TGCAATGATA CCGCGAGAAC CACGCTCACC GGCTCCAGAT
4201 TTATCAGCAA TAAACCAGCC AGCCGGAAGG GCCGAGCGCA GAAGTGGTCC TGCAACTTTA
4261 TCCGCCTCCA TCCAGTCTAT TAATTGTTGC CGGGAAGCTA GAGTAAGTAG TTCGCCAGTT
4321 AATAGTTTGC GCAACGTTGT TGCCATTGCT ACAGGCATCG TGGTGTCACG CTCGTCGTTT
4381 GGTATGGCTT CATTCAGCTC CGGTTCCCAA CGATCAAGGC GAGTTACATG ATCCCCCATG
4441 TTGTGCAAAA AAGCGGTTAG CTCCTTCGGT CCTCCGATCG TTGTCAGAAG TAAGTTGGCC
4501 GCAGTGTTAT CACTCATGGT TATGGCAGCA CTGCATAATT CTCTTACTGT CATGCCATCC
4561 GTAAGATGCT TTTCTGTGAC TGGTGAGTAC TCAACCAAGT CATTCTGAGA ATAGTGTATG
4621 CGGCGACCGA GTTGCTCTTG CCCGGCGTCA ATACGGGATA ATACCGCGCC ACATAGCAGA
4681 ACTTTAAAAG TGCTCATCAT TGGAAAACGT TCTTCGGGGC GAAAACTCTC AAGGATCTTA
4741 CCGCTGTTGA GATCCAGTTC GATGTAACCC ACTCGTGCAC CCAACTGATC TTCAGCATCT
4801 TTTACTTTCA CCAGCGTTTC TGGGTGAGCA AAAACAGGAA GGCAAAATGC CGCAAAAAAG
4861 GGAATAAGGG CGACACGGAA ATGTTGAATA CTCATACTCT TCCTTTTTCA ATATTATTGA
4921 AGCATTTATC AGGGTTATTG TCTCATGAGC GGATACATAT TTGAATGTAT TTAGAAAAAT
4981 AAACAAATAG GGGTTCCGCG CACATTTCCC CGAAAAGTGC CAC
E3L-FRT mCherry Kan Plasmid nucleic acid sequence
(SEQ ID NO: 31)
ATAGCGTCCCTAGGACGAACTACTGCCATTAATATCTCTATTATAGCTTCTGGACATAATTCATC
TATTATACCAGAATTAATGGGAACTATTCCGTATCTATCTAACATAGTTTTAAGAAAGTCAGAAT
CTAAGACCTGATGTTCATATATTGGTTCATACATGAAATGATCTCTATTGATGATAGTGACTATT
TCATTCTCTGAAAATTGGTAACTCATTCTATATATGCTTTCCTTGTTGATGAAGGATAGAATATA
CTCAATAGAATTTGTACCAACAAACTGTTCTCTTATGAATCGTATATCATCATCTGAAATAATCA
TGTAAGGCATACATTTAACAATTAGAGACTTGTCTCCTGTTATCAATATACTATTCTTGTGATAA
TTTATGTGTGAGGCAAATTTGTCCACGTTCTTTAATTTTGTTATAGTAGATATCAAATCCAATGG
AGCTACAGTTCTTGGCTTAAACAGATATAGTTTTTCTGGAACGAATTCTACAACATTATTATAAA
GGACTTTGGGTAGATAAGTGGGATGAAATCCTATTTTAATTAATGCGATAGCCTTGTCCTCGTGC
AGATATCCAAACGCTTTTGTGATAGTATGGCATTCATTGTCTAGAAACGCTCTACGAATATCTGT
GACAGATATCATCTTTAGAGAATATACTAGTCGCGTTAATAGTACTACAATTTGTATTTTTTAAT
CTATCTCAATAAAAAAATTAATATGTATGATTCAATGTATAACTAAACTACTAACTGTTATTGAT
AACTAGAATCAGAATCTAATGATGACGTAACCAAGCTAGCGCGGTTAACCGCCTTTTTATCCAT
CAGGTGATCTGTTTTTATTGTGGAGTCTAGAACTAGTGGATCCCCCGGGCTGCAGGAATTCGAT
ATCAAGCTCAGGCCTAGATCTGTCGACTTCGAGCTTATTTATATTCCAAAAAAAAAAAATAAAA
TTTCAATTTTTAAGCTTTTGAAGTTCCTATACTTTCTAGAGAATAGGAACTTCCACTAATTCCAA
ACCCACCCGCTTTTTATAGTAAGTTTTTCACCCATAAATAATAAATACAATAATTAATTTCTCGT
AAAAGTAGAAAATATATTCTAATTTATTGCACGGTAAGGAAGTAGATCATAACTCGAGGAATTG
GGGATCTCTATAATCTCGCGCAACCTATTTTCCCCTCGAACACTTTTTAAGCCGTAGATAAACAG
GCTGGGACACTTCACACGCGTATGGTGAGCAAGGGCGAGGAGGATAACATGGCCATCATCAAG
GAGTTCATGCGCTTCAAGGTGCACATGGAGGGCTCCGTGAACGGCCACGAGTTCGAGATCGAG
GGCGAGGGCGAGGGCCGCCCCTACGAGGGCACCCAGACCGCCAAGCTGAAGGTGACCAAGGGT
GGCCCCCTGCCCTTCGCCTGGGACATCCTGTCCCCTCAGTTCATGTACGGCTCCAAGGCCTACGT
GAAGCACCCCGCCGACATCCCCGACTACTTGAAGCTGTCCTTCCCCGAGGGCTTCAAGTGGGAG
CGCGTGATGAACTTCGAGGACGGCGGCGTGGTGACCGTGACCCAGGACTCCTCCCTGCAGGAC
GGCGAGTTCATCTACAAGGTGAAGCTGCGCGGCACCAACTTCCCCTCCGACGGCCCCGTAATGC
AGAAGAAGACCATGGGCTGGGAGGCCTCCTCCGAGCGGATGTACCCCGAGGACGGCGCCCTGA
AGGGCGAGATCAAGCAGAGGCTGAAGCTGAAGGACGGCGGCCACTACGACGCTGAGGTCAAG
ACCACCTACAAGGCCAAGAAGCCCGTGCAGCTGCCCGGCGCCTACAACGTCAACATCAAGTTG
GACATCACCTCCCACAACGAGGACTACACCATCGTGGAACAGTACGAACGCGCCGAGGGCCGC
CACTCCACCGGCGGCATGGACGAGCTGTACAAGTAATGAAGTTCCTATACTTTCTAGAGAATAG
GAACTTCCGGTTAACCGGTGATATAGGCAACCAGCAATAAAACTAATTTATTTTATCATTTTTTT
ATTCATCATCCTCTGGTGGTTCGTCGTTTCTATCGAATGTGGATCTGATTAACCCGTCATCTATA
GGTGATGCTGGTTCTGGAGATTCTGGAGGAGATGGATTATTATCTGGAAGAATCTCTGTTATTTC
CTTGTTTTCATGTATCGATTGCGTTGTAACATTAAGATTGCGAAATGCTCTAAATTTGGGAGGCT
TAAAGTGTTGTTTGCAATCTCTACACGCATGTCTAACTAGTGGAGGTTCGTCAGCGGCTCTAGTT
TGAATCATCATCGGCGTAGTATTCCTACTTTTACAGTTAGGACACGGTGTATTGTATTTCTCGTC
GAGAACGTTAAAATAATCGTTGTAACTCACATCCTTTATTTTATCTATATTGTATTCTACTCCTTT
CTTAATGCATTTTATACCGAATAAGAGATAGCGAAGGAATTCTTTTTCGGTGCCGCTAGTACCCT
TAATCATATCACATAGTGTTTTATATTCCAAATTTGTGGCAATAGACGGTTTATTTCTATACGAT
AGTTTGTTTCTGGAATCCTTTGAGTATTCTATACCAATATTATTCTTTGATTCGAATTTAGTTTCT
TCGATATTAGATTTTGTATTACCTATATTCTTGATGTAGTACTTTGATGATTTTTCCATGGCCCAT
TCTATCACGTAGAAAGCCAGTCCGCAGAAACGGTGCTGACCCCGGATGAATGTCAGCTACTGGG
CTATCTGGACAAGGGAAAACGCAAGCGCAAAGAGAAAGCAGGTAGCTTGCAGTGGGCTTACAT
GGCGATAGCTAGACTGGGCGGTTTTATGGACAGCAAGCGAACCGGAATTGCCAGCTGGGGCGC
CCTCTGGTAAGGTTGGGAAGCCCTGCAAAGTAAACTGGATGGCTTTCTCGCCGCCAAGGATCTG
ATGGCGCAGGGGATCAAGCTCTGATCAAGAGACAGGATGAGGATCGTTTCGCATGATTGAACA
AGATGGATTGCACGCAGGTTCTCCGGCCGCTTGGGTGGAGAGGCTATTCGGCTATGACTGGGCA
CAACAGACAATCGGCTGCTCTGATGCCGCCGTGTTCCGGCTGTCAGCGCAGGGGCGCCCGGTTC
TTTTTGTCAAGACCGACCTGTCCGGTGCCCTGAATGAACTGCAAGACGAGGCAGCGCGGCTATC
GTGGCTGGCCACGACGGGCGTTCCTTGCGCAGCTGTGCTCGACGTTGTCACTGAAGCGGGAAGG
GACTGGCTGCTATTGGGCGAAGTGCCGGGGCAGGATCTCCTGTCATCTCACCTTGCTCCTGCCG
AGAAAGTATCCATCATGGCTGATGCAATGCGGCGGCTGCATACGCTTGATCCGGCTACCTGCCC
ATTCGACCACCAAGCGAAACATCGCATCGAGCGAGCACGTACTCGGATGGAAGCCGGTCTTGTC
GATCAGGATGATCTGGACGAAGAGCATCAGGGGCTCGCGCCAGCCGAACTGTTCGCCAGGCTC
AAGGCGAGCATGCCCGACGGCGAGGATCTCGTCGTGACCCATGGCGATGCCTGCTTGCCGAATA
TCATGGTGGAAAATGGCCGCTTTTCTGGATTCATCGACTGTGGCCGGCTGGGTGTGGCGGACCG
CTATCAGGACATAGCGTTGGCTACCCGTGATATTGCTGAAGAGCTTGGCGGCGAATGGGCTGAC
CGCTTCCTCGTGCTTTACGGTATCGCCGCTCCCGATTCGCAGCGCATCGCCTTCTATCGCCTTCTT
GACGAGTTCTTCTGAATTATTAACGCTTACAATTTCCTGATGCGGTATTTTCTCCTTACGCATCTG
TGCGGTATTTCACACCGCATACAGGTGGCACTTTTCGGGGAAATGTGCGCGGAACCCCTATTTTC
GTTCCACTGAGCGTCAGACCCCGTAGAAAAGATCAAAGGATCTTCTTGAGATCCTTTTTTTCTGC
GCGTAATCTGCTGCTTGCAAACAAAAAAACCACCGCTACCAGCGGTGGTTTGTTTGCCGGATCA
AGAGCTACCAACTCTTTTTCCGAAGGTAACTGGCTTCAGCAGAGCGCAGATACCAAATACTGTC
CTTCTAGTGTAGCCGTAGTTAGGCCACCACTTCAAGAACTCTGTAGCACCGCCTACATACCTCGC
TCTGCTAATCCTGTTACCAGTGGCTGCTGCCAGTGGCGATAAGTCGTGTCTTACCGGGTTGGACT
CAAGACGATAGTTACCGGATAAGGCGCAGCGGTCGGGCTGAACGGGGGGTTCGTGCACACAGC
CCAGCTTGGAGCGAACGACCTACACCGAACTGAGATACCTACAGCGTGAGCTATGAGAAAGCG
CCACGCTTCCCGAAGGGAGAAAGGCGGACAGGTATCCGGTAAGCGGCAGGGTCGGAACAGGA
GAGCGCACGAGGGAGCTTCCAGGGGGAAACGCCTGGTATCTTTATAGTCCTGTCGGGTTTCGCC
ACCTCTGACTTGAGCGTCGATTTTTGTGATGCTCGTCAGGGGGGCGGAGCCTATGGAAAAACGC
CAGCAACGCGGCCTTTTTACGGTTCCTGGGCTTTTGCTGGCCTTTTGCTCACATGTTCTTTCCTGC
GTTATCCCCTGATTCTGTGGATAACCGTATTACCGCCTTTGAGTGAGCTGATACCGCTCGCCGCA
GCCGAACGACCGAGCGCAGCGAGTCA
E5R-FRT GFP Kan plasmid nucleic acid sequence
(SEQ ID NO: 32)
TAGTGGAGGTTCGTCAGCGGCTCTAGTTTGAATCATCATCGGCGTAGTATTCCTACTTTTACAGT
TAGGACACGGTGTATTGTATTTCTCGTCGAGAACGTTAAAATAATCGTTGTAACTCACATCCTTT
ATTTTATCTATATTGTATTCTACTCCTTTCTTAATGCATTTTATACCGAATAAGAGATAGCGAAG
GAATTCTTTTTCGGTGCCGCTAGTACCCTTAATCATATCACATAGTGTTTTATATTCCAAATTTGT
GGCAATAGACGGTTTATTTCTATACGATAGTTTGTTTCTGGAATCCTTTGAGTATTCTATACCAA
TATTATTCTTTGATTCGAATTTAGTTTCTTCGATATTAGATTTTGTATTACCTATATTCTTGATGTA
GTACTTTGATGATTTTTCCATGGCCCATTCTATTAAGTCTTCCAAGTTGGCATCATCCACATATTG
TGATAGTAATTCTCGGATATCAGTAGCGGCTACCGCCATTGATGTTTGTTCATTGGATGAGTAAC
TACTAATGTATACATTTTCCATTTATAACACTTATGTATTAACTTTGTTCATTTATATTTTTTCATT
ATTATGTTGATATTAACAAAAGTGAATATAAGCTAGCGCGGTTAACCGCCTTTTTATCCATCAGG
TGATCTGTTTTTATTGTGGAGTCTAGAACTAGTGGATCCCCCGGGCTGCAGGAATTCGATATCAA
GCTCAGGCCTAGATCTGTCGACTTCGAGCTTATTTATATTCCAAAAAAAAAAAATAAAATTTCA
ATTTTTAAGCTTTTGAAGTTCCTATACTTTCTAGAGAATAGGAACTTCCACTAATTCCAAACCCA
CCCGCTTTTTATAGTAAGTTTTTCACCCATAAATAATAAATACAATAATTAATTTCTCGTAAAAG
TAGAAAATATATTCTAATTTATTGCACGGTAAGGAAGTAGATCATAACTCGAGGAATTGGGGAT
CTCTATAATCTCGCGCAACCTATTTTCCCCTCGAACACTTTTTAAGCCGTAGATAAACAGGCTGG
GACACTTCACACGCGTATGGTGAGCAAGGGCGAGGAGCTGTTCACCGGGGTGGTGCCCATCCTG
GTCGAGCTGGACGGCGACGTAAACGGCCACAAGTTCAGCGTGTCCGGCGAGGGCGAGGGCGAT
GCCACCTACGGCAAGCTGACCCTGAAGTTCATCTGCACCACCGGCAAGCTGCCCGTGCCCTGGC
CCACCCTCGTGACCACCCTGACCTACGGCGTGCAGTGCTTCAGCCGCTACCCCGACCACATGAA
GCAGCACGACTTCTTCAAGTCCGCCATGCCCGAAGGCTACGTCCAGGAGCGCACCATCTTCTTC
AAGGACGACGGCAACTACAAGACCCGCGCCGAGGTGAAGTTCGAGGGCGACACCCTGGTGAAC
CGCATCGAGCTGAAGGGCATCGACTTCAAGGAGGACGGCAACATCCTGGGGCACAAGCTGGAG
TACAACTACAACAGCCACAACGTCTATATCATGGCCGACAAGCAGAAGAACGGCATCAAGGTG
AACTTCAAGATCCGCCACAACATCGAGGACGGCAGCGTGCAGCTCGCCGACCACTACCAGCAG
AACACCCCCATCGGCGACGGCCCCGTGCTGCTGCCCGACAACCACTACCTGAGCACCCAGTCCA
AGCTGAGCAAAGACCCCAACGAGAAGCGCGATCACATGGTCCTGCTGGAGTTCGTGACCGCCG
CCGGGATCACTCTCGGCATGGACGAGCTGTACAAGTAATGAAGTTCCTATACTTTCTAGAGAAT
AGGAACTTCCGGTTAACCGGTGATATTTCATCTTTCTCCAATACTAATTCAAATTGTTAAATTAA
TAATGGATAGTATAAATAGTTATTAGTGATAAAATAGTAAAAATAATTATTAGAATAAGAGTGT
AGTATCATAGATAACTCTCTTCTATAAAAATGGATTTTATTCGTAGAAAGTATCTTATATACACA
GTAGAAAATAATATAGATTTTTTAAAGGATGATACATTAAGTAAAGTAAACAATTTTACCCTCA
ATCATGTACTAGCTCTCAAGTATCTAGTTAGCAATTTTCCTCAACACGTTATTACTAAGGATGTA
TTAGCTAATACCAATTTTTTTGTTTTCATACATATGGTACGATGTTGTAAAGTGTACGAAGCGGT
TTTACGACACGCATTTGATGCACCCACGTTGTACGTTAAAGCATTGACTAAGAATTATTTATCGT
TTAGTAACGCAATACAATCGTACAAGGAAACCGTGCATAAACTAACACAAGATGAAAAATTTTT
AGAGGTTGCCGAATACATGGACGAATTAGGAGAACTTATAGGCGTAAATTATGACTTAGTTCTT
AATCCATTATTTCACGGAGGGGAACCCATCAAAGATATGGAAATCATTTTTTTAAAACTGTTTA
AGAAAACAGACTTCAAAGTTGTTAAAAAATTAAGTGTTATAAGATTACTTATTTGGGCTTACCT
AAGCAAGAAAGATACAGGTCACGTAGAAAGCCAGTCCGCAGAAACGGTGCTGACCCCGGATGA
ATGTCAGCTACTGGGCTATCTGGACAAGGGAAAACGCAAGCGCAAAGAGAAAGCAGGTAGCTT
GCAGTGGGCTTACATGGCGATAGCTAGACTGGGCGGTTTTATGGACAGCAAGCGAACCGGAATT
GCCAGCTGGGGCGCCCTCTGGTAAGGTTGGGAAGCCCTGCAAAGTAAACTGGATGGCTTTCTCG
CCGCCAAGGATCTGATGGCGCAGGGGATCAAGCTCTGATCAAGAGACAGGATGAGGATCGTTT
CGCATGATTGAACAAGATGGATTGCACGCAGGTTCTCCGGCCGCTTGGGTGGAGAGGCTATTCG
GCTATGACTGGGCACAACAGACAATCGGCTGCTCTGATGCCGCCGTGTTCCGGCTGTCAGCGCA
GGGGCGCCCGGTTCTTTTTGTCAAGACCGACCTGTCCGGTGCCCTGAATGAACTGCAAGACGAG
GCAGCGCGGCTATCGTGGCTGGCCACGACGGGCGTTCCTTGCGCAGCTGTGCTCGACGTTGTCA
CTGAAGCGGGAAGGGACTGGCTGCTATTGGGCGAAGTGCCGGGGCAGGATCTCCTGTCATCTCA
CCTTGCTCCTGCCGAGAAAGTATCCATCATGGCTGATGCAATGCGGCGGCTGCATACGCTTGAT
CCGGCTACCTGCCCATTCGACCACCAAGCGAAACATCGCATCGAGCGAGCACGTACTCGGATGG
AAGCCGGTCTTGTCGATCAGGATGATCTGGACGAAGAGCATCAGGGGCTCGCGCCAGCCGAAC
TGTTCGCCAGGCTCAAGGCGAGCATGCCCGACGGCGAGGATCTCGTCGTGACCCATGGCGATGC
CTGCTTGCCGAATATCATGGTGGAAAATGGCCGCTTTTCTGGATTCATCGACTGTGGCCGGCTGG
GTGTGGCGGACCGCTATCAGGACATAGCGTTGGCTACCCGTGATATTGCTGAAGAGCTTGGCGG
CGAATGGGCTGACCGCTTCCTCGTGCTTTACGGTATCGCCGCTCCCGATTCGCAGCGCATCGCCT
TCTATCGCCTTCTTGACGAGTTCTTCTGAATTATTAACGCTTACAATTTCCTGATGCGGTATTTTC
TCCTTACGCATCTGTGCGGTATTTCACACCGCATACAGGTGGCACTTTTCGGGGAAATGTGCGCG
GAACCCCTATTTTCGTTCCACTGAGCGTCAGACCCCGTAGAAAAGATCAAAGGATCTTCTTGAG
ATCCTTTTTTTCTGCGCGTAATCTGCTGCTTGCAAACAAAAAAACCACCGCTACCAGCGGTGGTT
TGTTTGCCGGATCAAGAGCTACCAACTCTTTTTCCGAAGGTAACTGGCTTCAGCAGAGCGCAGA
TACCAAATACTGTCCTTCTAGTGTAGCCGTAGTTAGGCCACCACTTCAAGAACTCTGTAGCACCG
CCTACATACCTCGCTCTGCTAATCCTGTTACCAGTGGCTGCTGCCAGTGGCGATAAGTCGTGTCT
TACCGGGTTGGACTCAAGACGATAGTTACCGGATAAGGCGCAGCGGTCGGGCTGAACGGGGGG
TTCGTGCACACAGCCCAGCTTGGAGCGAACGACCTACACCGAACTGAGATACCTACAGCGTGA
GCTATGAGAAAGCGCCACGCTTCCCGAAGGGAGAAAGGCGGACAGGTATCCGGTAAGCGGCAG
GGTCGGAACAGGAGAGCGCACGAGGGAGCTTCCAGGGGGAAACGCCTGGTATCTTTATAGTCC
TGTCGGGTTTCGCCACCTCTGACTTGAGCGTCGATTTTTGTGATGCTCGTCAGGGGGGCGGAGCC
TATGGAAAAACGCCAGCAACGCGGCCTTTTTACGGTTCCTGGGCTTTTGCTGGCCTTTTGCTCAC
ATGTTCTTTCCTGCGTTATCCCCTGATTCTGTGGATAACCGTATTACCGCCTTTGAGTGAGCTGAT
ACCGCTCGCCGCAGCCGAACGACCGAGCGCAGCGAGTCA
C11L-FRT mCherry Kan plasmid nucleic acid sequence
(SEQ ID NO: 33)
ATTGGAGACGTAACAATGTAGCGCATTGTTTCCTCGTCTATCTATATGTTTTGATAAGTTGTGAC
ACGTTTCAATTTCTAGTTTTATTTTTTTGTACGTCACATCTTCATCCAGTAGACGACATAGAATAC
ATGTGCAATCCATAGCTATTCTGGTGCTAATTATTCCTCATAAGATGATAAAAAGTGTAGTGAG
AGAGCATGAAGGAGATTTAGTATTTAGCAGTGCGGATATGATCCAAGAGGGTGAGATAGTCGTT
CTCGTTCAGAATCTTTCGCAGCATAAGTAGTATGTCGATATACTTATCGTTGAAGACTCTTCCAG
AGACGATAGCTGATTGAGTACAAAGTCCAATGATTGCACGAAGTTCTTCGGCGGTTTTCATGGA
GTCATTTCTGATGAAACATTTAATGATCTAAATTTCAGTTTATGTTTGTACCCCGTATTCATACTT
AACAAATTGGTATTACATACCATTAATAATGCAAGCATAAAAAATCGTTAGTAGATGTTTCTAA
ATATAGGTTCCGTAAGCTAGCGCGGTTAACCGCCTTTTTATCCATCAGGTGATCTGTTTTTATTG
TGGAGTCTAGAACTAGTGGATCCCCCGGGCTGCAGGAATTCGATATCAAGCTCAGGCCTAGATC
TGTCGACTTCGAGCTTATTTATATTCCAAAAAAAAAAAATAAAATTTCAATTTTTAAGCTTTTGA
AGTTCCTATACTTTCTAGAGAATAGGAACTTCCACTAATTCCAAACCCACCCGCTTTTTATAGTA
AGTTTTTCACCCATAAATAATAAATACAATAATTAATTTCTCGTAAAAGTAGAAAATATATTCTA
ATTTATTGCACGGTAAGGAAGTAGATCATAACTCGAGGAATTGGGGATCTCTATAATCTCGCGC
AACCTATTTTCCCCTCGAACACTTTTTAAGCCGTAGATAAACAGGCTGGGACACTTCACACGCGT
ATGGTGAGCAAGGGCGAGGAGGATAACATGGCCATCATCAAGGAGTTCATGCGCTTCAAGGTG
CACATGGAGGGCTCCGTGAACGGCCACGAGTTCGAGATCGAGGGCGAGGGCGAGGGCCGCCCC
TACGAGGGCACCCAGACCGCCAAGCTGAAGGTGACCAAGGGTGGCCCCCTGCCCTTCGCCTGG
GACATCCTGTCCCCTCAGTTCATGTACGGCTCCAAGGCCTACGTGAAGCACCCCGCCGACATCC
CCGACTACTTGAAGCTGTCCTTCCCCGAGGGCTTCAAGTGGGAGCGCGTGATGAACTTCGAGGA
CGGCGGCGTGGTGACCGTGACCCAGGACTCCTCCCTGCAGGACGGCGAGTTCATCTACAAGGTG
AAGCTGCGCGGCACCAACTTCCCCTCCGACGGCCCCGTAATGCAGAAGAAGACCATGGGCTGG
GAGGCCTCCTCCGAGCGGATGTACCCCGAGGACGGCGCCCTGAAGGGCGAGATCAAGCAGAGG
CTGAAGCTGAAGGACGGCGGCCACTACGACGCTGAGGTCAAGACCACCTACAAGGCCAAGAAG
CCCGTGCAGCTGCCCGGCGCCTACAACGTCAACATCAAGTTGGACATCACCTCCCACAACGAGG
ACTACACCATCGTGGAACAGTACGAACGCGCCGAGGGCCGCCACTCCACCGGCGGCATGGACG
AGCTGTACAAGTAATGAAGTTCCTATACTTTCTAGAGAATAGGAACTTCCGGTTCACTCGACGA
ACTAAACTACCTATACAAGATATGGTTGTGCCATAATTTTTATAAATTTTTTTTATGAGTATTTTT
ACAAAAATGTATAAAGTGTATGTCTTATGTATATTTATAAAAATGCTAAATATGCGATGTATCTA
TGTTATTTGTATTTATCTAAACAATACCTCTACCTCTAGATATTATACAAAAATTTTTTATTTCGG
CATATTAAAGTAAAATCTAGTTACCTTGAAAATGAATACAGTGGGTGGTTCCGTATCACCAGTA
AGAACATAATAGTCGAATACAGTATCCGATTGAGATTTTGCATACAATACTAGTCTAGAAAGAA
ATTTGTAATCATCTTCTGTGACGGGAGTCCATATATCTGTATCATCGTCCCATGCTATATTCCTGT
TATCATCATTAGTTAATGAAAATAACTCTCGTGCTTCAGAAAAGTCAAATATTGTATCCATACAT
ACATCTCCAAAACTATCGCTTATACGTTTATCTTTAACGATACCTATACCTAGATGGTTATTTAC
TAACAGACATTTTCCAGATCTATTGACTATAACTCCTATAGTTTCCACATCAACCAAGTAATGAT
CATCTATTGTTATATAACAATAACATAACTCTTTTCCGTTTTTATCAGTATGTATATCTATATCAA
CGTCGTCGTTGTAGTGAGCTCCTTTTCACGTAGAAAGCCAGTCCGCAGAAACGGTGCTGACCCC
GGATGAATGTCAGCTACTGGGCTATCTGGACAAGGGAAAACGCAAGCGCAAAGAGAAAGCAGG
TAGCTTGCAGTGGGCTTACATGGCGATAGCTAGACTGGGCGGTTTTATGGACAGCAAGCGAACC
GGAATTGCCAGCTGGGGCGCCCTCTGGTAAGGTTGGGAAGCCCTGCAAAGTAAACTGGATGGCT
TTCTCGCCGCCAAGGATCTGATGGCGCAGGGGATCAAGCTCTGATCAAGAGACAGGATGAGGA
TCGTTTCGCATGATTGAACAAGATGGATTGCACGCAGGTTCTCCGGCCGCTTGGGTGGAGAGGC
TATTCGGCTATGACTGGGCACAACAGACAATCGGCTGCTCTGATGCCGCCGTGTTCCGGCTGTC
AGCGCAGGGGCGCCCGGTTCTTTTTGTCAAGACCGACCTGTCCGGTGCCCTGAATGAACTGCAA
GACGAGGCAGCGCGGCTATCGTGGCTGGCCACGACGGGCGTTCCTTGCGCAGCTGTGCTCGACG
TTGTCACTGAAGCGGGAAGGGACTGGCTGCTATTGGGCGAAGTGCCGGGGCAGGATCTCCTGTC
ATCTCACCTTGCTCCTGCCGAGAAAGTATCCATCATGGCTGATGCAATGCGGCGGCTGCATACG
CTTGATCCGGCTACCTGCCCATTCGACCACCAAGCGAAACATCGCATCGAGCGAGCACGTACTC
GGATGGAAGCCGGTCTTGTCGATCAGGATGATCTGGACGAAGAGCATCAGGGGCTCGCGCCAG
CCGAACTGTTCGCCAGGCTCAAGGCGAGCATGCCCGACGGCGAGGATCTCGTCGTGACCCATGG
CGATGCCTGCTTGCCGAATATCATGGTGGAAAATGGCCGCTTTTCTGGATTCATCGACTGTGGCC
GGCTGGGTGTGGCGGACCGCTATCAGGACATAGCGTTGGCTACCCGTGATATTGCTGAAGAGCT
TGGCGGCGAATGGGCTGACCGCTTCCTCGTGCTTTACGGTATCGCCGCTCCCGATTCGCAGCGC
ATCGCCTTCTATCGCCTTCTTGACGAGTTCTTCTGAATTATTAACGCTTACAATTTCCTGATGCGG
TATTTTCTCCTTACGCATCTGTGCGGTATTTCACACCGCATACAGGTGGCACTTTTCGGGGAAAT
GTGCGCGGAACCCCTATTTTCGTTCCACTGAGCGTCAGACCCCGTAGAAAAGATCAAAGGATCT
TCTTGAGATCCTTTTTTTCTGCGCGTAATCTGCTGCTTGCAAACAAAAAAACCACCGCTACCAGC
GGTGGTTTGTTTGCCGGATCAAGAGCTACCAACTCTTTTTCCGAAGGTAACTGGCTTCAGCAGA
GCGCAGATACCAAATACTGTCCTTCTAGTGTAGCCGTAGTTAGGCCACCACTTCAAGAACTCTG
TAGCACCGCCTACATACCTCGCTCTGCTAATCCTGTTACCAGTGGCTGCTGCCAGTGGCGATAA
GTCGTGTCTTACCGGGTTGGACTCAAGACGATAGTTACCGGATAAGGCGCAGCGGTCGGGCTGA
ACGGGGGGTTCGTGCACACAGCCCAGCTTGGAGCGAACGACCTACACCGAACTGAGATACCTA
CAGCGTGAGCTATGAGAAAGCGCCACGCTTCCCGAAGGGAGAAAGGCGGACAGGTATCCGGTA
AGCGGCAGGGTCGGAACAGGAGAGCGCACGAGGGAGCTTCCAGGGGGAAACGCCTGGTATCTT
TATAGTCCTGTCGGGTTTCGCCACCTCTGACTTGAGCGTCGATTTTTGTGATGCTCGTCAGGGGG
GCGGAGCCTATGGAAAAACGCCAGCAACGCGGCCTTTTTACGGTTCCTGGGCTTTTGCTGGCCT
TTTGCTCACATGTTCTTTCCTGCGTTATCCCCTGATTCTGTGGATAACCGTATTACCGCCTTTGAG
TGAGCTGATACCGCTCGCCGCAGCCGAACGACCGAGCGCAGCGAGTCA
```

In some embodiments, engineered MVAΔE5R virus is generated by inserting an expression construct such as those illustrated by SEQ ID NOs: 22-24 and 32 (Table 2) into the MVA genomic region that corresponds to the position of the E5R locus (e.g., position 38,432 to 39,385 of SEQ ID NO: 1; or position 38,389 to 39,389 of the sequence set forth in Genflank Accession No. AY603355). In some embodiments, the MVAΔE5R virus is further engineered by inserting an expression construct such as that illustrated by SEQ ID NO: 31 into the MVA genomic region that corresponds to the E3L locus (e.g., position 36, 931 to 37,497 of the sequence set forth in GenBank Accession No. AY603355). In some embodiments, the MVAΔE5R virus is further engineered by inserting an expression construct such as that illustrated by SEQ ID NO: 33 into the MVA genomic region that corresponds to the C11R locus (e.g., position 4,160-4,785 of the sequence set forth in GenBank Accession No. AY603355).

A non-limiting example of an MVAΔK7R construct open reading frame according to the present technology is shown in SEQ ID NO: 25 (Table 3).

TABLE 3
Exemplary nucleotide sequence for the open reading frames of the
recombinant MVAΔK7R construct of the present technology.

```
pUC57-MVA-ΔK7R-mCherry vector nucleic acid sequence
(SEQ ID NO: 25)
      1 TCGCGCGTTT CGGTGATGAC GGTGAAAACC TCTGACACAT GCAGCTCCCG GAGACGGTCA
    61 CAGCTTGTCT GTAAGCGGAT GCCGGGAGCA GACAAGCCCG TCAGGGCGCG TCAGCGGGTG
  121 TTGGCGGGTG TCGGGGCTGG CTTAACTATG CGGCATCAGA GCAGATTGTA CTGAGAGTGC
  181 ACCATATGCG GTGTGAAATA CCGCACAGAT GCGTAAGGAG AAAATACCGC ATCAGGCGCC
  241 ATTCGCCATT CAGGCTGCGC AACTGTTGGG AAGGGCGATC GGTGCGGGCC TCTTCGCTAT
  301 TACGCCAGCT GGCGAAAGGG GGATGTGCTG CAAGGCGATT AAGTTGGGTA ACGCCAGGGT
  361 TTTCCCAGTC ACGACGTTGT AAAACGACGG CCAGTGAATT Cttgctgtaa acaagtttgg
  421 attatcgtaa gaggctagta tagaaattgt tgctcccatg gaatgaccca ataagtagat
  481 ttaatagtta ccacgtgctg taccaaagtc atcaatcatc attttttcac cattacttct
  541 tccatgtcca atatgatcat gtgagaatac taaaattcct aacgatgata tgttttcagc
  601 tagttcgtca taacgtccag aatgtttacc agctccatga cttatgaata ctaatgcctt
  661 aggatatgta atcattgtcc agattgaaca tacagtttgc actcatgatt cacgttatat
  721 aactatcaat attaacagtt cgtttgatga tcatattatt tttatgtttt attgataatt
  781 gtaaaaacat acaattaaat caatatagag gaaggagacg gctactgtct tttgtgagat
  841 agtcgatatc tcactaattc caaacccacc cgctttttat agtaagtttt tcacccataa
  901 ataataaata caataattaa tttctcgtaa aagtagaaaa tatattctaa tttattgcac
  961 ggtaaggaag tagatcataa ctcgacatgg tgagcaaggg cgaggaggat aacatggcca
1021 tcatcaagga gttcatgcgc ttcaaggtgc acatggaggg ctccgtgaac ggccacgagt
1081 tcgagatcga gggcgagggc gagggccgcc cctacgaggg cacccagacc gccaagctga
1141 aggtgaccaa gggtggcccc ctgcccttcg cctgggacat cctgtcccct cagttcatgt
1201 acggctccaa ggcctacgtg aagcaccccg ccgacatccc cgactacttg aagctgtcct
1261 tccccgaggg cttcaagtgg gagcgcgtga tgaacttcga ggacggcggc gtggtgaccg
1321 tgacccagga ctcctccctg caggacggcg agttcatcta caaggtgaag ctgcgcggca
1381 ccaacttccc ctccgacggc cccgtaatgc agaagaagac catgggctgg gaggcctcct
1441 ccgagcggat gtaccccgag gacggcgccc tgaagggcga gatcaagcag aggctgaagc
1501 tgaaggacgg cggccactac gacgctgagg tcaagaccac ctacaaggcc aagaagcccg
1561 tgcagctgcc cggcgcctac aacgtcaaca tcaagttgga catcacctcc cacaacgagg
1621 actacaccat cgtggaacag tacgaacgcg ccgagggccg ccactccacc ggcggcatgg
1681 acgagctGAT CACGAATTgt taactgatat aggggtcttc ataacgcata attattacgt
1741 tagcattcta tatccgtgtt aaaaaaaatt atcctatcat gtatttgaga gttttatatg
1801 tagcaaacat gatagctgtg atgccaataa gctttagata ttcacgcgtg ctagtgttag
1861 ggatggtatt atctggtggt gaaatgtccg ttatataatc tacaaaacaa tcatcgcata
1921 tagtatgcga tagtagagta aacattttta tagtttttac tggattcata catcgtctac
1981 ccaattcggt tatgaatgaa attgtcgcca atcttacacc caaccccttg ttatccatta
2041 gtatagtatt aacttcgtta tttatgtcat aaactgtaaa tgattttgta gatgccatat
2101 catacatgat attcatgtcc ctattataat cAAGCTTGGC GTAATCATGG TCATAGCTGT
2161 TTCCTGTGTG AAATTGTTAT CCGCTCACAA TTCCACACAA CATACGAGCC GGAAGCATAA
2221 AGTGTAAAGC CTGGGGTGCC TAATGAGTGA GCTAACTCAC ATTAATTGCG TTGCGCTCAC
2281 TGCCCGCTTT CCAGTCGGGA AACCTGTCGT GCCAGCTGCA TTAATGAATC GGCCAACGCG
2341 CGGGGAGAGG CGGTTTGCGT ATTGGGCGCT CTTCCGCTTC CTCGCTCACT GACTCGCTGC
2401 GCTCGGTCGT TCGGCTGCGG CGAGCGGTAT CAGCTCACTC AAAGGCGGTA ATACGGTTAT
2461 CCACAGAATC AGGGGATAAC GCAGGAAAGA ACATGTGAGC AAAAGGCCAG CAAAAGGCCA
2521 GGAACCGTAA AAAGGCCGCG TTGCTGGCGT TTTTCCATAG GCTCCGCCCC CCTGACGAGC
2581 ATCACAAAAA TCGACGCTCA AGTCAGAGGT GGCGAAACCC GACAGGACTA TAAAGATACC
2641 AGGCGTTTCC CCCTGGAAGC TCCCTCGTGC GCTCTCCTGT TCCGACCCTG CCGCTTACCG
2701 GATACCTGTC CGCCTTTCTC CCTTCGGGAA GCGTGGCGCT TTCTCATAGC TCACGCTGTA
2761 GGTATCTCAG TTCGGTGTAG GTCGTTCGCT CCAAGCTGGG CTGTGTGCAC GAACCCCCCG
2821 TTCAGCCCGA CCGCTGCGCC TTATCCGGTA ACTATCGTCT TGAGTCCAAC CCGGTAAGAC
2881 ACGACTTATC GCCACTGGCA GCAGCCACTG GTAACAGGAT TAGCAGAGCG AGGTATGTAG
2941 GCGGTGCTAC AGAGTTCTTG AAGTGGTGGC CTAACTACGG CTACACTAGA AGAACAGTAT
3001 TTGGTATCTG CGCTCTGCTG AAGCCAGTTA CCTTCGGAAA AAGAGTTGGT AGCTCTTGAT
3061 CCGGCAAACA AACCACCGCT GGTAGCGGTG GTTTTTTTGT TTGCAAGCAG CAGATTACGC
3121 GCAGAAAAAA AGGATCTCAA GAAGATCCTT TGATCTTTTC TACGGGGTCT GACGCTCAGT
3181 GGAACGAAAA CTCACGTTAA GGGATTTTGG TCATGAGATT ATCAAAAAGG ATCTTCACCT
3241 AGATCCTTTT AAATTAAAAA TGAAGTTTTA AATCAATCTA AAGTATATAT GAGTAAACTT
3301 GGTCTGACAG TTACCAATGC TTAATCAGTG AGGCACCTAT CTCAGCGATC TGTCTATTTC
3361 GTTCATCCAT AGTTGCCTGA CTCCCCGTCG TGTAGATAAC TACGATACGG GAGGGCTTAC
3421 CATCTGGCCC CAGTGCTGCA ATGATACCGC GAGACCCACG CTCACCGGCT CCAGATTTAT
3481 CAGCAATAAA CCAGCCAGCC GGAAGGGCCG AGCGCAGAAG TGGTCCTGCA ACTTTATCCG
3541 CCTCCATCCA GTCTATTAAT TGTTGCCGGG AAGCTAGAGT AAGTAGTTCG CCAGTTAATA
3601 GTTTGCGCAA CGTTGTTGCC ATTGCTACAG GCATCGTGGT GTCACGCTCG TCGTTTGGTA
3661 TGGCTTCATT CAGCTCCGGT TCCCAACGAT CAAGGCGAGT TACATGATCC CCCATGTTGT
3721 GCAAAAAAGC GGTTAGCTCC TTCGGTCCTC CGATCGTTGT CAGAAGTAAG TTGGCCGCAG
3781 TGTTATCACT CATGGTTATG GCAGCACTGC ATAATTCTCT TACTGTCATG CCATCCGTAA
3841 GATGCTTTTC TGTGACTGGT GAGTACTCAA CCAAGTCATT CTGAGAATAG TGTATGCGGC
3901 GACCGAGTTG CTCTTGCCCG GCGTCAATAC GGGATAATAC CGCGCCACAT AGCAGAACTT
3961 TAAAAGTGCT CATCATTGGA AAACGTTCTT CGGGGCGAAA ACTCTCAAGG ATCTTACCGC
4021 TGTTGAGATC CAGTTCGATG TAACCCACTC GTGCACCCAA CTGATCTTCA GCATCTTTTA
4081 CTTTCACCAG CGTTTCTGGG TGAGCAAAAA CAGGAAGGCA AAATGCCGCA AAAAAGGGAA
4141 TAAGGGCGAC ACGGAAATGT TGAATACTCA TACTCTTCCT TTTTCAATAT TATTGAAGCA
4201 TTTATCAGGG TTATTGTCTC ATGAGCGGAT ACATATTTGA ATGTATTTAG AAAAATAAAC
4261 AAATAGGGGT TCCGCGCACA TTTCCCCGAA AAGTGCCACC TGACGTCTAA GAAACCATTA
4321 TTATCATGAC ATTAACCTAT AAAAATAGGC GTATCACGAG GCCCTTTCGT C
```

### VACVΔC7L

The disclosure of the present technology relates to a C7L mutant vaccinia virus (i.e., VACVΔC7L; VACV comprising a C7L deletion; VACV genetically engineered to comprise a mutant C7L gene), or immunogenic compositions comprising the virus, in which the virus is engineered to express one or more specific genes of interest (SG), such as OX40L, and its use as a cancer immunotherapeutic (VACVΔC7L-OX40L). In some embodiments, the C7 gene of the vaccinia virus, through homologous recombination techniques, is engineered to contain a disruption comprising a heterologous nucleic acid sequence comprising one or more expression cassettes, which result in a C7 knockout such that the C7 gene is not expressed, expressed at levels so low as to have no effect, or the expressed protein is non-functional (e.g., is a null-mutation). In some embodiments, the ΔC7L mutant includes a heterologous nucleic acid sequence in place of all or a majority of the C7L gene sequence. For example, in some embodiments, the nucleic acid sequence corresponding to the position of C7 in the VACV genome (e.g., position 15,716 to 16,168 of SEQ ID NO: 2) is replaced with a heterologous nucleic acid sequence comprising an open reading frame that encodes a specific gene of interest (SG), such as human OX40L, resulting in VACVΔC7L-OX40L. In some embodiments, the expression cassette comprises a single open reading frame that encodes hFlt3L, resulting in VACVΔC7L-hFlt3L.

Additionally or alternatively, in some embodiments, VACVΔC7L is engineered to express both OX40L and hFlt3L. In some embodiments, the thymidine kinase (TK) gene of the vaccinia virus (e.g., position 80,962 to 81,032 of SEQ ID NO: 2), through homologous recombination, is engineered to contain a disruption comprising a heterologous nucleic acid sequence comprising one or more expression cassettes, which results in a TK gene knockout such that the TK gene is not expressed, expressed at levels so low as to have no effect, or the expressed protein is non-functional (e.g., is a null-mutation). The resulting VACVΔC7L-TK(−) virus is further engineered to comprise one or more expression cassettes that are flanked by a partial sequence of the TK gene (TK-L and TK-R) on either side. In some embodiments, the expression cassette comprises a single open reading frame that encodes a specific gene of interest (SG), such as OX40L using the vaccinia viral synthetic early and late promoter (PsE/L), resulting in VACVΔC7L-TK(−)-OX40L. In some embodiments, the recombinant virus is further modified at the C7 locus, through homologous recombination techniques, to contain a disruption comprising a heterologous nucleic acid sequence comprising one or more expression cassettes, which result in a C7 knockout such that the C7 gene is not expressed, expressed at levels so low as to have no effect, or the expressed protein is non-functional (e.g., is a null-mutation). In some embodiments, the expression cassette comprises a single open reading frame that encodes a specific gene of interest (SG), such as hFlt3L, resulting in VACVΔC7L-hFlt3L-TK(−)-OX40L. In some embodiments, the expression cassette encoding OX40L is inserted into the C7 locus while the expression cassette encoding hFlt3L is inserted into the TK locus. In some embodiments, a VACVΔC7L-anti-CTLA-4-hFlt3L-TK(−) virus is further modified to encode OX40L, resulting in VACVΔC7L-anti-CTLA-4-TK(−)-hFlt3L-OX40L. In some embodiments, the VACVΔC7L-anti-CTLA-4-TK(−)-hFlt3L-OX40L virus is further modified to express hIL-12, resulting in VACVΔC7L-anti-CTLA-4-TK(−)-hFlt3L-OX40L-hIL-12.

Additionally or alternatively, in some embodiments, the heterologous nucleic acid sequence comprises an expression cassette comprising two or more open reading frames encoding two or more specific genes of interest, separated by a nucleotide sequence that encodes, in the 5′ to 3′ direction, a protease cleavage site (e.g., a furin cleavage site) and a 2A peptide (Pep2A) sequence.

In some embodiments, the disclosure of the present technology provides a VACVΔC7L-TK(−)-anti-CTLA-4-OX40L virus. In some embodiments, the disclosure of the present technology provides a VACVΔC7L-E3LΔ83N-TK(−)-hFlt3L-anti-CTLA-4-OX40L virus.

In some embodiments, the recombinant VACVΔC7L-OX40L viruses described above are modified to express at least one further heterologous gene, such as any one or more of hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or include at least one other viral gene mutation or deletion, such as any one or more of the following deletions: E3L (ΔE3L); E3LΔ83N; B2R (ΔB2R), B19R (B18R; ΔWR200); E5R; K7R; C12L (IL18BP); B8R; B14R; N1L; C11R; K1L; M1L; N2L; and/or WR199. In other embodiments, no further heterologous genes are added other than those provided in the name of the virus herein (e.g., OX40L or OX40L and hFlt3L), and/or no further viral genes other than C7L or C7L and TK are disrupted or deleted.

Although in certain embodiments described above, the transgene may be inserted into the TK locus, splitting the TK gene and obliterating it, other suitable integration loci can be selected. For example, VACV encodes several immune modulatory genes, including but not limited to C11, K3, F1, F2, F4, F6, F8, F9, F11, F14.5, J2, A46, E3L, B18R (WR200), E5R, K7R, C12L, B8R, B14R, N1L, C11R, K1L, C16, M1L, N2L, and WR199. Accordingly, in some embodiments, these genes can be deleted to potentially enhance immune activating properties of the virus and allow insertion of transgenes.

In some embodiments, the heterologous nucleotide sequence further comprises an additional expression cassette comprising an open reading frame that encodes a selectable marker operably linked to a promoter that is capable of directing expression of the selectable marker. In some embodiments, the selectable marker is a xanthine-guanine phosphoribosyl transferase (gpt) gene. In some embodiments, the selectable marker is a green fluorescent protein (GFP) gene. In some embodiments, the selectable marker is an mCherry gene encoding a red fluorescent protein.

### VACVΔE5R

The disclosure of the present technology relates to a E5R mutant vaccinia virus (i.e., VACVΔE5R; VACV comprising an E5R deletion; VACV genetically engineered to comprise a mutant E5R gene), or immunogenic compositions comprising the virus, in which the virus is engineered to express one or more specific genes of interest (SG), such as OX40L, and its use as a cancer immunotherapeutic (VACVΔE5R-OX40L). In some embodiments, the E5R gene of the vaccinia virus, through homologous recombination techniques, is engineered to contain a disruption comprising a heterologous nucleic acid sequence comprising one or more expression cassettes, which result in an E5R knockout such that the E5R gene is not expressed, expressed at levels so low as to have no effect, or the expressed protein is non-functional (e.g., is a null-mutation). In some embodiments, the ΔE5R mutant includes a heterologous nucleic acid sequence in place of all or a majority of the E5R gene sequence. For example, in some embodiments, the nucleic acid sequence corresponding to the position of E5R in the VACV genome (e.g., position 49,236 to 50,261 of SEQ ID NO: 2) is replaced with a heterologous nucleic acid sequence comprising an open reading frame that encodes a specific gene of interest (SG), such as human OX40L, resulting in VACVΔE5R-OX40L. In some embodiments, the expression cassette comprises a single open reading frame that encodes hFlt3L, resulting in VACVΔE5R-hFlt3L.

Additionally or alternatively, in some embodiments, VACVΔE5R is engineered to express both OX40L and hFlt3L. In some embodiments, the thymidine kinase (TK) gene of the vaccinia virus (e.g., position 80,962 to 81,032 of SEQ ID NO: 2), through homologous recombination, is engineered to contain a disruption comprising a heterologous nucleic acid sequence comprising one or more expression cassettes, which results in a TK gene knockout such that the TK gene is not expressed, expressed at levels so low as to have no effect, or the expressed protein is non-functional (e.g., is a null-mutation). The resulting VACVΔE5R-TK(−) virus is further engineered to comprise one or more expression cassettes that are flanked by a partial sequence of the TK gene (TK-L and TK-R) on either side. In some embodiments, the expression cassette comprises a single open reading frame that encodes a specific gene of interest (SG), such as OX40L using the vaccinia viral synthetic early and late promoter (PsE/L), resulting in VACVΔE5R-TK(−)-OX40L. In some embodiments, the recombinant virus is further modified at the E5R locus, through homologous recombination techniques, to contain a disruption comprising a heterologous nucleic acid sequence comprising one or more expression cassettes, which result in an E5R knockout such that the E5R gene is not expressed, expressed at levels so low as to have no effect, or the expressed protein is non-functional (e.g., is a null-mutation). In some embodiments, the expression cassette comprises a single open reading frame that encodes a specific gene of interest (SG), such as hFlt3L, resulting in VACVΔE5R-hFlt3L-TK(−)-OX40L. In some embodiments, the expression cassette encoding OX40L is inserted into the E5R locus while the expression cassette encoding hFlt3L is inserted into the TK locus. In some embodiments, a VACVΔE5R-anti-CTLA-4-hFlt3L-TK(−) virus is further modified to encode OX40L, resulting in VACVΔE5R-anti-CTLA-4-TK(−)-hFlt3L-OX40L. In some embodiments, the VACVΔE5R-anti-CTLA-4-TK(−)-hFlt3L-OX40L virus is further modified to express hIL-12, resulting in VACVΔE5R-anti-CTLA-4-TK(−)-hFlt3L-OX40L-hIL-12. In some embodiments, the VACVΔE5R is engineered to comprise a nucleic acid encoding IL-15/IL-15Rα (VACVΔE5R-IL-15/IL-15Rα) alone or in combination with one or more additional modifications as described herein. For example, in some embodiments, the VACVΔE5R-IL-15/IL-15Rα is further engineered to comprise a nucleic acid encoding OX40L (VACVΔE5R-IL-15/IL-15Rα-OX40L).

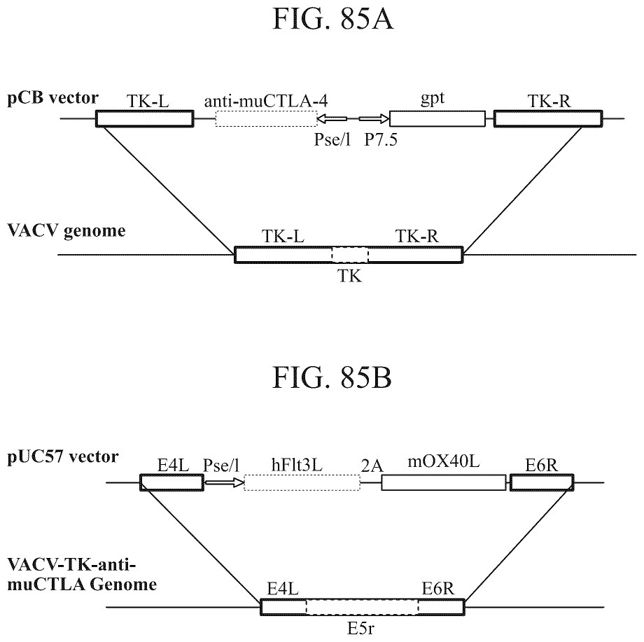

Additionally or alternatively, in some embodiments, the heterologous nucleic acid sequence comprises an expression cassette comprising two or more open reading frames encoding two or more specific genes of interest, separated by a nucleotide sequence that encodes, in the 5′ to 3′ direction, a protease cleavage site (e.g., a furin cleavage site) and a 2A peptide (Pep2A) sequence. For example, in some embodiments, the TK locus of the vaccinia genome is modified through homologous recombination to express both the heavy and light chain of an antibody, such as anti-CTLA-4, wherein the coding sequences of the heavy chain and light chain are separated by a cassette including a furin cleavage site followed by a 2A peptide (Pep2A) sequence to produce VACV-TK(−)-anti-CTLA-4. In some embodiments, the VACV-TK(−)-anti-CTLA-4 genome is further modified to comprise a deletion of E5R, in which all or a majority of the E5R gene sequence is replaced by a first specific gene of interest (e.g., hFlt3L) and a second specific gene of interest (e.g., OX40L), wherein the coding sequences of the first and second specific genes of interest are separated by a cassette including a furin cleavage site followed by a 2A peptide (Pep2A) sequence, thereby forming a recombinant virus such as VACV-TK⁻-anti-CTLA-4-E5R⁻-hFlt3L-OX40L (or VACVΔE5R-TK(−)-anti-CTLA-4-hFlt3L-OX40L) (see, e.g., FIG. 85A).

In some embodiments, the genetically engineered or recombinant VACVΔE5R viruses described above are modified to express at least one heterologous gene, such as any one or more of hOX40L, hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or include at least one other viral gene mutation or deletion, such as any one or more of the following deletions: E3L (ΔE3L); E3LΔ83N; C7 (ΔC7L); B2R (ΔB2R), B19R (B18R; ΔWR200); E5R; K7R; C12L (IL18BP); B8R; B14R; N1L; C11R; K1L; M1L; N2L; and/or WR199. In other embodiments, no further heterologous genes are added other than those provided in the name of the virus herein (e.g., OX40L or OX40L and hFlt3L), and/or no further viral genes other than E5R or E5R and TK are disrupted or deleted.

Although in certain embodiments described above, the transgene may be inserted into the TK locus, splitting the TK gene and obliterating it, other suitable integration loci can be selected. For example, VACV encodes several immune modulatory genes, including but not limited to C7, C11, K3, F1, F2, F4, F6, F8, F9, F11, F14.5, J2, A46, E3L, B18R (WR200), E5R, K7R, C12L, B8R, B14R, N1L, C11R, K1L, C16, M1L, N2L, and WR199. Accordingly, in some embodiments, these genes can be deleted to potentially enhance immune activating properties of the virus and allow insertion of transgenes.

In other embodiments, no further heterologous genes are added other than those provided in the name of the virus herein (e.g., OX40L), and/or no further viral genes other than E5R or E5R and TK are disrupted or deleted.

In some embodiments, the heterologous nucleotide sequence further comprises an additional expression cassette comprising an open reading frame that encodes a selectable marker operably linked to a promoter that is capable of directing expression of the selectable marker. In some embodiments, the selectable marker is a xanthine-guanine phosphoribosyl transferase (gpt) gene. In some embodiments, the selectable marker is a green fluorescent protein (GFP) gene. In some embodiments, the selectable marker is an mCherry gene encoding a red fluorescent protein.

Non-limiting examples of VACVΔE5R construct open reading frames according to the present technology are shown in SEQ ID NOs: 26-28 (Table 4).

TABLE 4
Exemplary nucleotide sequences for the open reading frames of the
recombinant VACVΔE5R constructs of the present technology.

```
pUC57-VACV-ΔE5R-mCherry vector nucleic acid sequence
(SEQ ID NO: 26)
      1 TCGCGCGTTT CGGTGATGAC GGTGAAAACC TCTGACACAT GCAGCTCCCG GAGACGGTCA
    61 CAGCTTGTCT GTAAGCGGAT GCCGGGAGCA GACAAGCCCG TCAGGGCGCG TCAGCGGGTG
  121 TTGGCGGGTG TCGGGGCTGG CTTAACTATG CGGCATCAGA GCAGATTGTA CTGAGAGTGC
  181 ACCATATGCG GTGTGAAATA CCGCACAGAT GCGTAAGGAG AAAATACCGC ATCAGGCGCC
  241 ATTCGCCATT CAGGCTGCGC AACTGTTGGG AAGGGCGATC GGTGCGGGCC TCTTCGCTAT
  301 TACGCCAGCT GGCGAAAGGG GGATGTGCTG CAAGGCGATT AAGTTGGGTA ACGCCAGGGT
  361 TTTCCCAGTC ACGACGTTGT AAAACGACGG CCAGTGAATT CGAGCTCGGT ACCgagatag
  421 cgaaggaatt ctttttcggt gccgctagta cccttaatca tatcacatag tgttttatat
  481 tccaaatttg tggcaataga cggtttattt ctatacgata gtttgtttct ggaatccttt
  541 gagtattcta taccaatatt attctttgat tcgaatttag tttcttcgat attagatttt
  601 gtattaccta tattcttgat gtagtacttt gatgattttt ccatggccca ttctattaag
  661 tcttccaagt tggcatcatc cacatattgt gatagtaatt ctcggatatc agtagcggtt
  721 accgccattg atgtttgttc attggatgag taactactaa tgtatacatt ttccatttat
  781 aacacttatg tattaacttt gttcatttat attttttcat tattaagctt gatcaggcct
  841 tcactaattc caaacccacc cgctttttat agtaagtttt tcacccataa ataataaata
  901 caataattaa tttctcgtaa aagtagaaaa tatattctaa tttattgcac ggtaaggaag
  961 tagatcataa ctcgacatgg tgagcaaggg cgaggaggat aacatggcca tcatcaagga
1021 gttcatgcgc ttcaaggtgc acatggaggg ctccgtgaac ggccacgagt tcgagatcga
1081 gggcgagggc gagggccgcc cctacgaggg cacccagacc gccaagctga aggtgaccaa
1141 gggtggcccc ctgcccttcg cctgggacat cctgtcccct cagttcatgt acggctccaa
1201 ggcctacgtg aagcaccccg ccgacatccc cgactacttg aagctgtcct tccccgaggg
1261 cttcaagtgg gagcgcgtga tgaacttcga ggacggcggc gtggtgaccg tgacccagga
1321 ctcctccctg caggacggcg agttcatcta caaggtgaag ctgcgcggca ccaacttccc
1381 ctccgacggc cccgtaatgc agaagaagac catgggctgg gaggcctcct ccgagcggat
1441 gtaccccgag gacggcgccc tgaagggcga gatcaagcag aggctgaagc tgaaggacgg
1501 cggccactac gacgctgagg tcaagaccac ctacaaggcc aagaagcccg tgcagctgcc
1561 cggcgcctac aacgtcaaca tcaagttgga catcacctcc cacaacgagg actacaccat
1621 cgtggaacag tacgaacgcg ccgagggccg ccactccacc ggcggcatgg acgagctGAT
1681 CACGAATTgt taacctgcat ttcatctttc tccaatacta attcaaattg ttaaattaat
1741 aatggatagt ataaatagtt attagtgata aaatagtaaa aataattatt agaataagag
1801 tgtagtatca tagataactc tcttctataa aaatggattt tattcgtaga aagtatctta
1861 tatacacagt agaaaataat atagattttt taaaggatga tacattaagt aaagtaaaca
1921 attttaccct caatcatgta ctagctctca agtatctagt tagcaatttt cctcaacatg
1981 ttattactaa ggatgtatta gctaatacca atttttttgt tttcatacat atggtacgat
2041 gttgtaaagt gtacgaagcg gttttacgac acgcatttga tgcacccacg ttgtacgtta
2101 aagcattgac taagaattat tGGATCCCGG GCCCGTCGAC TGCAGAGGCC TGCATGCAAG
2161 CTTGGCGTAA TCATGGTCAT AGCTGTTTCC TGTGTGAAAT TGTTATCCGC TCACAATTCC
2221 ACACAACATA CGAGCCGGAA GCATAAAGTG TAAAGCCTGG GGTGCCTAAT GAGTGAGCTA
2281 ACTCACATTA ATTGCGTTGC GCTCACTGCC CGCTTTCCAG TCGGGAAACC TGTCGTGCCA
2341 GCTGCATTAA TGAATCGGCC AACGCGCGGG GAGAGGCGGT TTGCGTATTG GGCGCTCTTC
2401 CGCTTCCTCG CTCACTGACT CGCTGCGCTC GGTCGTTCGG CTGCGGCGAG CGGTATCAGC
2461 TCACTCAAAG GCGGTAATAC GGTTATCCAC AGAATCAGGG GATAACGCAG GAAAGAACAT
2521 GTGAGCAAAA GGCCAGCAAA AGGCCAGGAA CCGTAAAAAG GCCGCGTTGC TGGCGTTTTT
2581 CCATAGGCTC CGCCCCCCTG ACGAGCATCA CAAAAATCGA CGCTCAAGTC AGAGGTGGCG
2641 AAACCCGACA GGACTATAAA GATACCAGGC GTTTCCCCCT GGAAGCTCCC TCGTGCGCTC
2701 TCCTGTTCCG ACCCTGCCGC TTACCGGATA CCTGTCCGCC TTTCTCCCTT CGGGAAGCGT
2761 GGCGCTTTCT CATAGCTCAC GCTGTAGGTA TCTCAGTTCG GTGTAGGTCG TTCGCTCCAA
2821 GCTGGGCTGT GTGCACGAAC CCCCCGTTCA GCCCGACCGC TGCGCCTTAT CCGGTAACTA
2881 TCGTCTTGAG TCCAACCCGG TAAGACACGA CTTATCGCCA CTGGCAGCAG CCACTGGTAA
2941 CAGGATTAGC AGAGCGAGGT ATGTAGGCGG TGCTACAGAG TTCTTGAAGT GGTGGCCTAA
3001 CTACGGCTAC ACTAGAAGAA CAGTATTTGG TATCTGCGCT CTGCTGAAGC CAGTTACCTT
3061 CGGAAAAAGA GTTGGTAGCT CTTGATCCGG CAAACAAACC ACCGCTGGTA GCGGTGGTTT
3121 TTTTGTTTGC AAGCAGCAGA TTACGCGCAG AAAAAAAGGA TCTCAAGAAG ATCCTTTGAT
3181 CTTTTCTACG GGGTCTGACG CTCAGTGGAA CGAAAACTCA CGTTAAGGGA TTTTGGTCAT
3241 GAGATTATCA AAAAGGATCT TCACCTAGAT CCTTTTAAAT TAAAAATGAA GTTTTAAATC
3301 AATCTAAAGT ATATATGAGT AAACTTGGTC TGACAGTTAC CAATGCTTAA TCAGTGAGGC
3361 ACCTATCTCA GCGATCTGTC TATTTCGTTC ATCCATAGTT GCCTGACTCC CCGTCGTGTA
3421 GATAACTACG ATACGGGAGG GCTTACCATC TGGCCCCAGT GCTGCAATGA TACCGCGAGA
3481 CCCACGCTCA CCGGCTCCAG ATTTATCAGC AATAAACCAG CCAGCCGGAA GGGCCGAGCG
3541 CAGAAGTGGT CCTGCAACTT TATCCGCCTC CATCCAGTCT ATTAATTGTT GCCGGGAAGC
3601 TAGAGTAAGT AGTTCGCCAG TTAATAGTTT GCGCAACGTT GTTGCCATTG CTACAGGCAT
3661 CGTGGTGTCA CGCTCGTCGT TTGGTATGGC TTCATTCAGC TCCGGTTCCC AACGATCAAG
3721 GCGAGTTACA TGATCCCCCA TGTTGTGCAA AAAAGCGGTT AGCTCCTTCG GTCCTCCGAT
3781 CGTTGTCAGA AGTAAGTTGG CCGCAGTGTT ATCACTCATG GTTATGGCAG CACTGCATAA
3841 TTCTCTTACT GTCATGCCAT CCGTAAGATG CTTTTCTGTG ACTGGTGAGT ACTCAACCAA
3901 GTCATTCTGA GAATAGTGTA TGCGGCGACC GAGTTGCTCT TGCCCGGCGT CAATACGGGA
3961 TAATACCGCG CCACATAGCA GAACTTTAAA AGTGCTCATC ATTGGAAAAC GTTCTTCGGG
4021 GCGAAAACTC TCAAGGATCT TACCGCTGTT GAGATCCAGT TCGATGTAAC CCACTCGTGC
4081 ACCCAACTGA TCTTCAGCAT CTTTTACTTT CACCAGCGTT TCTGGGTGAG CAAAAACAGG
4141 AAGGCAAAAT GCCGCAAAAA AGGGAATAAG GGCGACACGG AAATGTTGAA TACTCATACT
4201 CTTCCTTTTT CAATATTATT GAAGCATTTA TCAGGGTTAT TGTCTCATGA GCGGATACAT
4261 ATTTGAATGT ATTTAGAAAA ATAAACAAAT AGGGGTTCCG CGCACATTTC CCCGAAAAGT
4321 GCCACCTGAC GTCTAAGAAA CCATTATTAT CATGACATTA ACCTATAAAA ATAGGCGTAT
4381 CACGAGGCCC TTTCGTC
pUC57-VACV-ΔE5R-hFlt3L-hOX40L vector nucleic acid sequence
(SEQ ID NO: 27)
      1 tcgcgcgttt cggtgatgac ggtgaaaacc tctgacacat gcagctcccg gagacggtca
    61 cagcttgtct gtaagcggat gccgggagca gacaagcccg tcagggcgcg tcagcgggtg
  121 ttggcgggtg tcggggctgg cttaactatg cggcatcaga gcagattgta ctgagagtgc
  181 accatatgcg gtgtgaaata ccgcacagat gcgtaaggag aaaataccgc atcaggcgcc
  241 attcgccatt caggctgcgc aactgttggg aagggcgatc ggtgcgggcc tcttcgctat
  301 tacgccagct ggcgaaaggg ggatgtgctg caaggcgatt aagttgggta acgccagggt
  361 tttcccagtc acgacgttgt aaaacgacgg ccagtgaatt cgagctcggt accgagatag
  421 cgaaggaatt ctttttcggt gccgctagta cccttaatca tatcacatag tgttttatat
  481 tccaaatttg tggcaataga cggtttattt ctatacgata gtttgtttct ggaatccttt
  541 gagtattcta taccaatatt attctttgat tcgaatttag tttcttcgat attagatttt
  601 gtattaccta tattcttgat gtagtacttt gatgattttt ccatggccca ttctattaag
  661 tcttccaagt tggcatcatc cacatattgt gatagtaatt ctcggatatc agtagcggtt
  721 accgccattg atgtttgttc attggatgag taactactaa tgtatacatt ttccatttat
  781 aacacttatg tattaacttt gttcatttat attttttcat tattaagctt ggatccaaaa
  841 attgaaattt tatttttttt ttttggaata taaataagct cgaagtcgac gaattcatga
  901 cagtactagc tccagcttgg tccccgacaa cataccttct actactacta ttgctatcct
  961 ccggactatc tggaacccag gattgctctt ttcagcactc tccgatctcg tctgatttcg
1021 cggttaagat cagagagcta tccgactact tgctacagga ttacccagta accgtcgcgt
1081 ccaacctaca agatgaagaa ctatgtggtg gactttggag actagtccta gcgcaaagat
1141 ggatggaaag acttaagacc gtagcgggat ctaagatgca gggactacta gaaagagtca
1201 acaccgagat ccacttcgtc acaaagtgtg cgtttcaacc accaccgtcc tgtctaagat
1261 tcgtccagac aaacatctcc agactactac aagaaacctc cgagcagcta gtagcgctaa
1321 aaccgtggat cacaagacag aacttctcga gatgtctaga gctacagtgt cagccggatt
1381 cttctacatt accaccacca tggtcaccaa gaccactaga agctacagct ccaactgctc
1441 cacaaccacc attgctactt ttgctattgc tacccgtcgg attgctacta ttagctgctg
1501 cttggtgtct acactggcag agaactagaa gaagaactcc aagaccggga gaacaagtac
1561 caccagtacc atctccacag gacctactac tagtcgagca cagaagaaga agaagatcgg
1621 gagcgaccaa cttctcgcta ttgaaacaag cgggagatgt cgaagaaaat ccgggaccaa
1681 tggaaagagt acagccgcta gaagaaaacg taggaaatgc ggctagaccg agattcgaga
1741 gaaacaagct actattggtc gcgtccgtca tccaaggact aggattgcta ttgtgcttca
1801 cctacatctg cctacacttc tccgcgctac aagtctctca tagatacccg agaatccagt
1861 ccatcaaggt ccagttcacc gagtacaaga aagagaaggg attcatccta acctcgcaga
1921 aagaggacga gatcatgaag gtccagaaca actccgtcat catcaactgc gacggattct
1981 acctaatctc cctaaaggga tacttctccc aagaagtcaa catctccttg cactaccaga
2041 aggatgagga accgctattc cagctaaaga aagtcagatc cgtcaactcc ctaatggtcg
2101 cctctctaac gtacaaggac aaggtctacc taaacgtcac caccgacaac acatccctag
2161 atgatttcca cgtaaacggt ggagagctaa tcctaatcca tcagaacccg ggagagttct
2221 gtgtattata agttaacctg catttcatct ttctccaata ctaattcaaa ttgttaaatt
2281 aataatggat agtataaata gttattagtg ataaaatagt aaaaataatt attagaataa
2341 gagtgtagta tcatagataa ctctcttcta taaaaatgga ttttattcgt agaaagtatc
2401 ttatatacac agtagaaaat aatatagatt ttttaaagga tgatacatta agtaaagtaa
2461 acaattttac cctcaatcat gtactagctc tcaagtatct agttagcaat tttcctcaac
2521 atgttattac taaggatgta ttagctaata ccaatttttt tgttttcata catatggtac
2581 gatgttgtaa agtgtacgaa gcggttttac gacacgcatt tgatgcaccc acgttgtacg
2641 ttaaagcatt gactaagaat tattggatcc cgggcccgtc gaccaagctt ggcgtaatca
2701 tggtcatagc tgtttcctgt gtgaaattgt tatccgctca caattccaca caacatacga
2761 gccggaagca taaagtgtaa agcctggggt gcctaatgag tgagctaact cacattaatt
2821 gcgttgcgct cactgcccgc tttccagtcg ggaaacctgt cgtgccagct gcattaatga
2881 atcggccaac gcgcggggag aggcggtttg cgtattgggc gctcttccgc ttcctcgctc
2941 actgactcgc tgcgctcggt cgttcggctg cggcgagcgg tatcagctca ctcaaaggcg
3001 gtaatacggt tatccacaga atcaggggat aacgcaggaa agaacatgtg agcaaaaggc
3061 cagcaaaagg ccaggaaccg taaaaaggcc gcgttgctgg cgtttttcca taggctccgc
3121 ccccctgacg agcatcacaa aaatcgacgc tcaagtcaga ggtggcgaaa cccgacagga
3181 ctataaagat accaggcgtt tccccctgga agctccctcg tgcgctctcc tgttccgacc
3241 ctgccgctta ccggatacct gtccgccttt ctcccttcgg gaagcgtggc gctttctcat
3301 agctcacgct gtaggtatct cagttcggtg taggtcgttc gctccaagct gggctgtgtg
3361 cacgaacccc ccgttcagcc cgaccgctgc gccttatccg gtaactatcg tcttgagtcc
3421 aacccggtaa gacacgactt atcgccactg gcagcagcca ctggtaacag gattagcaga
3481 gcgaggtatg taggcggtgc tacagagttc ttgaagtggt ggcctaacta cggctacact
3541 agaagaacag tatttggtat ctgcgctctg ctgaagccag ttaccttcgg aaaaagagtt
3601 ggtagctctt gatccggcaa acaaaccacc gctggtagcg gtggtttttt tgtttgcaag
3661 cagcagatta cgcgcagaaa aaaaggatct caagaagatc ctttgatctt ttctacgggg
3721 tctgacgctc agtggaacga aaactcacgt taagggattt tggtcatgag attatcaaaa
3781 aggatcttca cctagatcct tttaaattaa aaatgaagtt ttaaatcaat ctaaagtata
3841 tatgagtaaa cttggtctga cagttaccaa tgcttaatca gtgaggcacc tatctcagcg
3901 atctgtctat ttcgttcatc catagttgcc tgactccccg tcgtgtagat aactacgata
3961 cgggagggct taccatctgg ccccagtgct gcaatgatac cgcgagaccc acgctcaccg
4021 gctccagatt tatcagcaat aaaccagcca gccggaaggg ccgagcgcag aagtggtcct
4081 gcaactttat ccgcctccat ccagtctatt aattgttgcc gggaagctag agtaagtagt
4141 tcgccagtta atagtttgcg caacgttgtt gccattgcta caggcatcgt ggtgtcacgc
4201 tcgtcgtttg gtatggcttc attcagctcc ggttcccaac gatcaaggcg agttacatga
4261 tcccccatgt tgtgcaaaaa agcggttagc tccttcggtc ctccgatcgt tgtcagaagt
4321 aagttggccg cagtgttatc actcatggtt atggcagcac tgcataattc tcttactgtc
4381 atgccatccg taagatgctt ttctgtgact ggtgagtact caaccaagtc attctgagaa
4441 tagtgtatgc ggcgaccgag ttgctcttgc ccggcgtcaa tacgggataa taccgcgcca
4501 catagcagaa ctttaaaagt gctcatcatt ggaaaacgtt cttcggggcg aaaactctca
4561 aggatcttac cgctgttgag atccagttcg atgtaaccca ctcgtgcacc caactgatct
4621 tcagcatctt ttactttcac cagcgtttct gggtgagcaa aaacaggaag gcaaaatgcc
4681 gcaaaaaagg gaataagggc gacacggaaa tgttgaatac tcatactctt cctttttcaa
4741 tattattgaa gcatttatca gggttattgt ctcatgagcg gatacatatt tgaatgtatt
4801 tagaaaaata aacaaatagg ggttccgcgc acatttcccc gaaaagtgcc acctgacgtc
4861 taagaaacca ttattatcat gacattaacc tataaaaata ggcgtatcac gaggcccttt
4921 cgtc
pUC57-VACV-ΔE5R-hFlt3L-mOX40L vector nucleic acid sequence
(SEQ ID NO: 28)
      1 tcgcgcgttt cggtgatgac ggtgaaaacc tctgacacat gcagctcccg gagacggtca
    61 cagcttgtct gtaagcggat gccgggagca gacaagcccg tcagggcgcg tcagcgggtg
  121 ttggcgggtg tcggggctgg cttaactatg cggcatcaga gcagattgta ctgagagtgc
  181 accatatgcg gtgtgaaata ccgcacagat gcgtaaggag aaaataccgc atcaggcgcc
  241 attcgccatt caggctgcgc aactgttggg aagggcgatc ggtgcgggcc tcttcgctat
  301 tacgccagct ggcgaaaggg ggatgtgctg caaggcgatt aagttgggta acgccagggt
  361 tttcccagtc acgacgttgt aaaacgacgg ccagtgaatt cgagctcggt accgagatag
  421 cgaaggaatt ctttttcggt gccgctagta cccttaatca tatcacatag tgttttatat
  481 tccaaatttg tggcaataga cggtttattt ctatacgata gtttgtttct ggaatccttt
  541 gagtattcta taccaatatt attctttgat tcgaatttag tttcttcgat attagatttt
  601 gtattaccta tattcttgat gtagtacttt gatgattttt ccatggccca ttctattaag
  661 tcttccaagt tggcatcatc cacatattgt gatagtaatt ctcggatatc agtagcggtt
  721 accgccattg atgtttgttc attggatgag taactactaa tgtatacatt ttccatttat
  781 aacacttatg tattaacttt gttcatttat attttttcat tattaagctt ggatccaaaa
  841 attgaaattt tatttttttt ttttggaata taaataagct cgaagtcgac gaattcatga
  901 cagtactagc tccagcttgg tccccgacaa cataccttct actactacta ttgctatcct
  961 ccggactatc tggaacccag gattgctctt ttcagcactc tccgatctcg tctgatttcg
1021 cggttaagat cagagagcta tccgactact tgctacagga ttacccagta accgtcgcgt
1081 ccaacctaca agatgaagaa ctatgtggtg gactttggag actagtccta gcgcaaagat
1141 ggatggaaag acttaagacc gtagcgggat ctaagatgca gggactacta gaaagagtca
1201 acaccgagat ccacttcgtc acaaagtgtg cgtttcaacc accaccgtcc tgtctaagat
1261 tcgtccagac aaacatctcc agactactac aagaaacctc cgagcagcta gtagcgctaa
1321 aaccgtggat cacaagacag aacttctcga gatgtctaga gctacagtgt cagccggatt
1381 cttctacatt accaccacca tggtcaccaa gaccactaga agctacagct ccaactgctc
1441 cacaaccacc attgctactt ttgctattgc tacccgtcgg attgctacta ttagctgctg
1501 cttggtgtct acactggcag agaactagaa gaagaactcc aagaccggga gaacaagtac
1561 caccagtacc atctccacag gacctactac tagtcgagca cagaagaaga agaagatcgg
1621 gagcgaccaa cttctcgcta ttgaaacaag cgggagatgt cgaagaaaat ccgggaccaa
1681 tggagggcga gggggtccag cctctggacg agaacctcga aaacgggtct cgccctcgct
1741 ttaaatggaa gaagactctt aggctcgttg taagcggcat caagggggcc ggtatgttgc
1801 tgtgcttcat atatgtgtgt ttgcaactta gctcttcacc tgcaaaagac ccccccatac
1861 aacgccttcg gggggctgtg acccgctgtg aagatggtca attgtttatt tcttcttaca
1921 agaacgagta tcagacgatg gaagtccaga ataactccgt agtgattaag tgtgacggac
1981 tgtacatcat ctacttgaaa ggatcttttt tccaggaggt caaaattgac ctccacttca
2041 gggaggatca caaccctatc tcaatcccta tgttgaacga cggcagaaga atcgtcttta
2101 ctgtagtcgc ttcactggcc ttcaaggata aggtgtactt gaccgtaaac gctcctgata
2161 ccttgtgcga gcatttgcaa atcaacgatg gagaacttat cgttgtccaa ctcacaccag
2221 gttactgtgc tcctgagggc agttatcaca gtacagtgaa ccaagtccca ctgtgagtta
2281 acctgcattt catctttctc caatactaat tcaaattgtt aaattaataa tggatagtat
2341 aaatagttat tagtgataaa atagtaaaaa taattattag aataagagtg tagtatcata
2401 gataactctc ttctataaaa atggatttta ttcgtagaaa gtatcttata tacacagtag
2461 aaaataatat agatttttta aaggatgata cattaagtaa agtaaacaat tttaccctca
2521 atcatgtact agctctcaag tatctagtta gcaattttcc tcaacatgtt attactaagg
2581 atgtattagc taataccaat ttttttgttt tcatacatat ggtacgatgt tgtaaagtgt
2641 acgaagcggt tttacgacac gcatttgatg cacccacgtt gtacgttaaa gcattgacta
2701 agaattattg gatcccgggc ccgtcgacca agcttggcgt aatcatggtc atagctgttt
2761 cctgtgtgaa attgttatcc gctcacaatt ccacacaaca tacgagccgg aagcataaag
2821 tgtaaagcct ggggtgccta atgagtgagc taactcacat taattgcgtt gcgctcactg
2881 cccgctttcc agtcgggaaa cctgtcgtgc cagctgcatt aatgaatcgg ccaacgcgcg
2941 gggagaggcg gtttgcgtat tgggcgctct tccgcttcct cgctcactga ctcgctgcgc
3001 tcggtcgttc ggctgcggcg agcggtatca gctcactcaa aggcggtaat acggttatcc
3061 acagaatcag gggataacgc aggaaagaac atgtgagcaa aaggccagca aaaggccagg
3121 aaccgtaaaa aggccgcgtt gctggcgttt ttccataggc tccgcccccc tgacgagcat
3181 cacaaaaatc gacgctcaag tcagaggtgg cgaaacccga caggactata aagataccag
3241 gcgtttcccc ctggaagctc cctcgtgcgc tctcctgttc cgaccctgcc gcttaccgga
3301 tacctgtccg cctttctccc ttcgggaagc gtggcgcttt ctcatagctc acgctgtagg
3361 tatctcagtt cggtgtaggt cgttcgctcc aagctgggct gtgtgcacga accccccgtt
3421 cagcccgacc gctgcgcctt atccggtaac tatcgtcttg agtccaaccc ggtaagacac
3481 gacttatcgc cactggcagc agccactggt aacaggatta gcagagcgag gtatgtaggc
3541 ggtgctacag agttcttgaa gtggtggcct aactacggct acactagaag aacagtattt
3601 ggtatctgcg ctctgctgaa gccagttacc ttcggaaaaa gagttggtag ctcttgatcc
3661 ggcaaacaaa ccaccgctgg tagcggtggt ttttttgttt gcaagcagca gattacgcgc
3721 agaaaaaaag gatctcaaga agatcctttg atcttttcta cggggtctga cgctcagtgg
3781 aacgaaaact cacgttaagg gattttggtc atgagattat caaaaaggat cttcacctag
3841 atccttttaa attaaaaatg aagttttaaa tcaatctaaa gtatatatga gtaaacttgg
3901 tctgacagtt accaatgctt aatcagtgag gcacctatct cagcgatctg tctatttcgt
3961 tcatccatag ttgcctgact ccccgtcgtg tagataacta cgatacggga gggcttacca
4021 tctggcccca gtgctgcaat gataccgcga gacccacgct caccggctcc agatttatca
4081 gcaataaacc agccagccgg aagggccgag cgcagaagtg gtcctgcaac tttatccgcc
4141 tccatccagt ctattaattg ttgccgggaa gctagagtaa gtagttcgcc agttaatagt
4201 ttgcgcaacg ttgttgccat tgctacaggc atcgtggtgt cacgctcgtc gtttggtatg
4261 gcttcattca gctccggttc ccaacgatca aggcgagtta catgatcccc catgttgtgc
4321 aaaaaagcgg ttagctcctt cggtcctccg atcgttgtca gaagtaagtt ggccgcagtg
4381 ttatcactca tggttatggc agcactgcat aattctctta ctgtcatgcc atccgtaaga
4441 tgcttttctg tgactggtga gtactcaacc aagtcattct gagaatagtg tatgcggcga
4501 ccgagttgct cttgcccggc gtcaatacgg gataataccg cgccacatag cagaacttta
4561 aaagtgctca tcattggaaa acgttcttcg gggcgaaaac tctcaaggat cttaccgctg
4621 ttgagatcca gttcgatgta acccactcgt gcacccaact gatcttcagc atcttttact
4681 ttcaccagcg tttctgggtg agcaaaaaca ggaaggcaaa atgccgcaaa aaagggaata
4741 agggcgacac ggaaatgttg aatactcata ctcttccttt ttcaatatta ttgaagcatt
4801 tatcagggtt attgtctcat gagcggatac atatttgaat gtatttagaa aaataaacaa
4861 ataggggttc cgcgcacatt tccccgaaaa gtgccacctg acgtctaaga aaccattatt
4921 atcatgacat taacctataa aaataggcgt atcacgaggc cctttcgtc
```

In some embodiments, engineered VACVΔE5R virus is generated by inserting an expression construct such as those illustrated by SEQ ID NOs: 26-28 (Table 4) into the VACV genomic region that corresponds to the position of the E5R locus (e.g., position 49,236 to 50,261 of NO SEQ ID NO: 2).

A non-limiting example of an anti-CTLA-4 antibody open reading for insertion into the TK locus of VACV, using, e.g., a pCB plasmid-based vector, is shown in SEQ ID NO: 29 (Table 5).

TABLE 5
Exemplary nucleotide sequence for the open
reading frames of the vaccinia virus constructs
of the present technology.

anti-muCTLA4-muIgG2a nucleotide sequence.
(SEQ ID NO: 29)
5′ATGGAATGGTCCTTTGTCTTTCTTTTTTTCTTGTCCGCAGCTGCCGGA
GTACATTCGGAGGCGAAGTTGCAAGAGTCCGGACCTGTACTTGTTAAGCC
CGGAGCTTCAGTGAAAATGTCCTGTAAAGCATCCGGATATACCTTTACAG
ATTATTATATGAATTGGGTGAAGCAAAGTCATGGAAAGAGTCTTGAATGG
ATAGGAGTAATTAATCCTTATAACGGAGATACATCTTATAATCAAAAGTT
CAAAGGAAAAGCTACACTAACTGTTGATAAATCCTCAAGTACTGCTTATA
TGGAACTAAACTCACTAACTAGTGAAGATTCTGCAGTTTATTATTGTGCT
CGTTATTATGGTTCGTGGTTTGCATATTGGGGACAGGGAACCTTAATAAC
TGTAAGTACAGCAAAAACAACGGCGCCTTCTGTTTATCCATTAGCGCCTG
TATGTGGAGATACAACTGGTTCTTCTGTTACATTAGGATGTCTAGTCAAA
GGATATTTCCCAGAACCTGTTACATTAACCTGGAACTCCGGTTCGCTATC
ATCAGGTGTACACACTTTCCCGGCGGTTCTACAATCTGATTTGTATACAT
TATCATCTTCCGTTACAGTTACTTCTTCCACTTGGCCATCGCAAAGTATC
ACATGTAACGTAGCGCACCCAGCTTCATCAACAAAAGTCGATAAAAAAAT
AGAGCCGCGAGGTCCCACTATAAAGCCGTGTCCACCTTGTAAATGTCCAG
CTCCTAATTTATTAGGAGGACCCAGTGTATTTATTTTCCCTCCTAAAATT
AAAGATGTATTGATGATTTCTTTATCTCCAATTGTTACATGCGTGGTTGT
AGATGTATCCGAAGACGATCCGGATGTGCAAATATCGTGGTTCGTTAATA
ATGTGGAAGTTCACACCGCGCAAACTCAAACTCACAGAGAGGATTACAAT
TCTACCTTGCGTGTAGTGTCGGCTCTACCTATACAACACCAAGATTGGAT
GTCTGGAAAAGAATTTAAATGCAAAGTTAATAACAAAGACCTTCCAGCGC
CAATAGAAAGAACAATATCCAAACCTAAAGGTAGTGTAAGAGCTCCTCAA
GTATACGTTTTACCGCCTCCTGAAGAAGAAATGACGAAAAAACAAGTTAC
ATTAACCTGTATGGTGACAGATTTTATGCCAGAGGATATTTATGTGGAGT
GGACTAATAATGGAAAAACGGAATTGAATTACAAAAATACTGAACCTGTA
TTAGATAGTGATGGATCATATTTTATGTACAGTAAATTGAGAGTGGAAAA
AAAGAATTGGGTTGAAAGAAATTCGTACTCTTGTTCAGTTGTACATGAGG
GACTACATAATCATCATACCACTAAGAGTTTTTCAAGAACCCCTGGTAAA
CGTAGAAGGCGTAGGAGATCTGGTGCTACTAATTTCTCCTTGTTAAAACA
AGCCGGTGACGTCGAAGAAAACCCTGGTCCTATGATGACATGGACTCTAC
TATTCCTTGCCTTCCTTCATCACTTAACAGGGTCATGTGCCGACATCGTT
ATGACCCAAACTACTCTTTCATTACCTGTGTCATTAGGAGACCAGGCATC
CATCTCATGCCGATCCTCTCAATCGATAGTACACTCTAATGGAAATACCT
ATTTAGAATGGTATTTGCAGAAGCCGGGTCAATCTCCGAAGTTACTAATT
TATAAAGTTTCTAACAGATTTTCAGGAGTTCCAGATAGATTTAGTGGTTC
AGGATCCGGTACTGACTTTACATTAAAGATCTCTCGTGTAGAAGCTGAGG
ATCTTGGTGTATATTATTGTTTCCAAGGATCTCACGTCCCATATACTTTC
GGAGGAGGAACCAAACTAGAAATTAAGCGTGCTGATGCGGCTCCCACAGT
AAGTATATTCCCACCGTCGTCAGAACAATTAACCTCGGGAGGAGCCTCGG
TCGTTTGTTTCTTAAATAACTTTTATCCTAAGGATATCAACGTTAAATGG
AAAATTGATGGTTCTGAACGTCAGAATGGAGTGTTGAATTCATGGACTGA
TCAAGATTCCAAAGACTCTACTTATTCGATGTCGAGTACACTTACTTTAA
CAAAAGATGAATATGAACGACATAACTCATATACTTGTGAAGCAACCCAT
AAGACATCTACTTCACCAATTGTAAAGTCTTTTAATCGAAACGAATGCTAA
ITALICS UPPER CASE = human IgG kappa leader sequence
UPPER CASE UNDERLINED = anti-CTLA-4 Heavy Chain
BOLD UPPER CASE = Furin cleavage site
BOLD UPPER CASE UNDERLINED = Pep2A
BOLD UPPER CASE ITALICS = anti-CTLA-4 Light Chain

Non-limiting examples of IL-12 expression constructs for insertion into, for example, the E5R locus, using, for example, a pUC57 vector, according to the present technology by which, for example, the VACV-E3LΔ83N-ΔTK-anti-CTLA-4-ΔE5R-hFlt3L-OX40L-hIL-12 construct is engineered are shown in SEQ ID NOs: 35-38 (Table 5A) (see also FIG. 169).

TABLE 5A
Exemplary nucleotide sequences for IL-12 expression constructs
of the present technology (human and murine IL-12 fusion proteins).

Human IL-12 nucleic acid sequence
(SEQ ID NO: 35)
ATGTGTCACCAGCAGCTAGTGATCTCGTGGTTCTCCCTAGTATTTCTAGCCTCTCCGCTAGTAGC
GATCTGGGAGCTAAAAAAGGATGTCTACGTCGTCGAGCTAGACTGGTATCCAGATGCTCCGGGA
GAAATGGTAGTCCTAACATGTGATACACCGGAAGAGGATGGAATCACCTGGACCTTGGATCAAT
CCTCCGAAGTACTAGGATCCGGAAAGACCCTAACCATCCAAGTCAAAGAATTCGGAGATGCGG
GACAGTACACCTGTCACAAAGGTGGAGAAGTCCTATCGCACTCCCTACTACTATTGCACAAGAA
AGAGGACGGTATCTGGTCCACCGATATCCTAAAGGATCAGAAAGAGCCGAAGAACAAGACCTT
CCTAAGATGCGAAGCGAAGAACTACTCCGGAAGATTCACATGTTGGTGGCTAACCACAATCTCC
ACCGACCTAACCTTCTCCGTCAAATCTTCTAGAGGATCCAGTGATCCGCAGGGTGTAACATGTG
GTGCTGCTACATTATCTGCGGAGAGAGTCAGAGGAGACAACAAAGAGTACGAGTACTCCGTCG
AGTGTCAAGAGGATTCTGCTTGTCCAGCTGCGGAAGAATCTCTACCGATCGAAGTAATGGTAGA
CGCGGTCCACAAGTTGAAGTACGAGAACTACACCTCCTCGTTCTTCATCAGAGACATCATCAAA
CCAGATCCGCCGAAGAATCTACAGCTAAAGCCGCTAAAGAACTCCAGACAGGTCGAAGTATCT
TGGGAATACCCGGATACTTGGTCTACACCGCACTCCTACTTTTCGTTGACCTTCTGTGTACAAGT
CCAGGGAAAGTCCAAGAGAGAGAAGAAGGACAGAGTCTTTACCGACAAGACATCCGCGACCGT
CATCTGTAGAAAGAACGCCTCTATTTCTGTCAGAGCGCAGGACAGATATTACTCCTCGTCTTGG
AGTGAATGGGCGTCTGTACCATGTTCCAGAAGAAGAAGAAGAAGAGCGACCAACTTCTCGCTA
TTGAAGCAAGCGGGAGATGTAGAAGAAAATCCGGGACCGTTAGATATGTGTCCGGCGAGATCT
CTACTACTAGTCGCGACACTAGTCCTACTAGACCATCTATCTCTAGCGAGAAATTTGCCAGTAGC
GACACCAGATCCTGGAATGTTTCCGTGTCTACACCACTCGCAGAATCTACTAAGAGCCGTGTCT
AACATGCTACAGAAGGCGAGACAGACCTTGGAATTCTACCCGTGTACCTCCGAAGAAATCGATC
ACGAGGATATCACCAAGGACAAGACCTCTACAGTCGAAGCTTGTCTACCGCTAGAGTTGACCAA
GAACGAGTCCTGCCTAAACTCCAGAGAAACCTCCTTCATCACCAACGGATCTTGCCTAGCGTCT
AGAAAGACCTCTTTCATGATGGCGCTATGCCTATCCTCTATCTACGAGGACCTAAAGATGTACC
AGGTCGAATTCAAGACCATGAACGCGAAGCTACTAATGGACCCGAAGAGACAGATCTTCTTGG
ACCAGAATATGCTAGCGGTCATCGACGAACTAATGCAGGCGCTAAACTTCAACTCTGAAACCGT
GCCGCAGAAGTCCAGTTTAGAAGAACCGGATTTCTACAAGACCAAGATCAAGCTATGCATCCTA
CTACACGCGTTCAGAATCAGAGCGGTCACCATCGATAGAGTCATGTCTTACCTAAACGCGTCCA
GAAGACCGAAAGGAAGAGGAAAGAGAAGAAGAGAAAAGCAAAGACCGACCGACTGCCATCTA
TGA
HuIL12p40-PEP2A-huIL12p35-AA-PIGF-AA amino acid sequence
(SEQ ID NO: 36)
MCHQQLVISWFSLVFLASPLVAIWELKKDVYVVELDWYPDAPGEMVVLTCDTPEEDGITW
TLDQSSEVLGSGKTLTIQVKEFGDAGQYTCHKGGEVLSHSLLLLHKKEDGIWSTDILKDQ
KEPKNKTFLRCEAKNYSGRFTCWWLTTISTDLTFSVKSSRGSSDPQGVTCGAATLSAERV
RGDNKEYEYSVECQEDSACPAAEESLPIEVMVDAVHKLKYENYTSSFFIRDIIKPDPPKN
LQLKPLKNSRQVEVSWEYPDTWSTPHSYFSLTFCVQVQGKSKREKKDRVFTDKTSATVIC
RKNASISVRAQDRYYSSSWSEWASVPCS
RRRRRRATNFSLLKQAGDVEENPGPLD
MCPARSLLLVATLVLLDHLSLARNLPVATPDPGMFPCLHHSQNLLRAVSNMLQKARQTLE
FYPCTSEEIDHEDITKDKTSTVEACLPLELTKNESCLNSRETSFITNGSCLASRKTSEMM
ALCLSSIYEDLKMYQVEFKTMNAKLLMDPKRQIFLDQNMLAVIDELMQALNENSETVPQK
SSLEEPDFYKTKIKLCILLHAFRIRAVTIDRVMSYLNAS
RRPKGRGKRRREKQRPTDCHL
UNDERLINED = PEP2A sequence
BOLD UNDERLINED = Matrix binding tag
Murine IL-12 nucleic acid sequence
(SEQ ID NO: 37)
ATGTGTCCACAAAAGCTCACCATCTCATGGTTCGCTATAGTATTGCTCGTTTCCCCATTGATGGC
AATGTGGGAGCTTGAAAAAGATGTGTATGTAGTGGAGGTTGACTGGACTCCTGATGCACCCGGA
GAGACAGTCAATTTGACATGCGACACCCCCGAAGAAGATGATATTACATGGACATCCGACCAA
CGGCACGGGGTCATCGGATCTGGGAAGACCTTGACAATTACAGTGAAGGAGTTCTTGGATGCAG
GACAGTACACATGTCATAAAGGGGGCGAGACACTCTCACATTCACATCTGCTCCTGCATAAAAA
GGAGAACGGAATCTGGTCCACCGAAATCCTTAAGAATTTCAAGAACAAAACCTTTCTTAAGTGT
GAGGCCCCTAATTATTCCGGAAGATTTACATGCAGCTGGTTGGTCCAGCGCAATATGGACCTCA
AATTTAATATCAAGTCTTCTTCCAGCTCCCCAGATTCTCGGGCAGTGACTTGCGGCATGGCATCC
CTCTCCGCTGAGAAAGTAACATTGGATCAACGAGACTACGAGAAGTACTCTGTTAGTTGTCAAG
AGGACGTTACATGCCCTACCGCAGAAGAGACATTGCCAATTGAATTGGCCCTTGAAGCACGCCA
GCAGAATAAGTACGAAAATTACAGTACAAGCTTTTTCATCCGAGACATAATTAAACCCGATCCT
CCTAAGAATCTCCAAATGAAACCTTTGAAGAATAGCCAAGTGGAAGTTTCATGGGAGTATCCAG
ATTCCTGGTCCACACCACATTCCTATTTCTCCCTTAAGTTCTTCGTCAGAATCCAGAGGAAGAAA
GAAAAGATGAAGGAAACCGAGGAAGGCTGCAACCAGAAGGGCGCCTTTCTGGTAGAGAAAAC
CAGCACTGAGGTTCAGTGTAAGGGGGGAAACGTGTGTGTGCAGGCACAAGATCGATACTACAA
CTCAAGCTGTAGTAAATGGGCCTGCGTACCTTGTCGGGTTCGATCCAGACGACGCCGGAGACGA
GCTACCAATTTTTCCTTGCTCAAGCAGGCAGGCGATGTGGAGGAAAACCCAGGGCCCCTTGACA
TGTGTCAGAGCCGGTACCTCCTTTTCCTCGCAACCCTGGCTCTGCTTAACCACCTCTCACTGGCT
AGGGTAATTCCCGTATCTGGGCCTGCCCGATGCCTCAGCCAGAGTCGGAATCTCCTTAAGACCA
CAGACGATATGGTAAAAACAGCAAGGGAGAAACTCAAACATTACTCTTGTACAGCAGAGGACA
TCGATCATGAAGACATAACCCGGGACCAGACCTCAACATTGAAAACTTGTCTGCCACTGGAGCT
TCATAAGAACGAGTCCTGCCTTGCCACACGAGAGACCTCTAGCACTACACGGGGGTCCTGCCTG
CCTCCACAGAAAACCTCCTTGATGATGACCCTGTGTCTCGGCAGTATTTACGAAGATTTGAAGA
TGTACCAAACAGAGTTTCAGGCCATTAACGCAGCATTGCAAAACCATAATCACCAGCAGATAAT
CCTTGATAAGGGTATGCTGGTAGCCATCGACGAACTTATGCAATCTCTGAATCATAATGGCGAA
ACTCTGCGACAAAAGCCCCCAGTTGGAGAAGCCGACCCCTACCGAGTCAAGATGAAACTCTGC
ATACTCCTGCATGCCTTTTCCACACGGGTTGTTACTATCAATCGAGTCATGGGGTATCTTTCTTC
AGCACGGCGCCCTAAAGGGCGCGGAAAACGCCGCCGGGAAAAACAAAGACCTACTGATTGCCA
TCTGTGA
Murine IL-12 fusion protein amino acid sequence
(SEQ ID NO: 38)
MCPQKLTISWFAIVLLVSPLMAMWELEKDVYVVEVDWTPDAPGETVNLTCDTPEEDDITWTSDQRH
GVIGSGKTLTITVKEFLDAGQYTCHKGGETLSHSHLLLHKKENGIWSTEILKNEKNKTFLKCEAPN
YSGRFTCSWLVQRNMDLKENIKSSSSSPDSRAVTCGMASLSAEKVTLDORDYEKYSVSCQEDVTCP
TAEETLPIELALEARQQNKYENYSTSFFIRDIIKPDPPKNLQMKPLKNSQVEVSWEYPDSWSTPHS
YFSLKFFVRIQRKKEKMKETEEGCNQKGAFLVEKTSTEVQCKGGNVCVQAQDRYYNSSCSKWACVP
CRVRSRRRRRRATNFSLLKQAGDVEENPGPLDMCQSRYLLFLATLALLNHLSLARVIPVSGPARCL
SQSRNLLKTTDDMVKTAREKLKHYSCTAEDIDHEDITRDQTSTLKTCLPLELHKNESCLATRETSS
TTRGSCLPPQKTSLMMTLCLGSIYEDLKMYQTEFQAINAALQNHNHQQIILDKGMLVAIDELMQSL
NHNGETLRQKPPVGEADPYRVKMKLCILLHAFSTRVVTINRVMGYLSSARRPKGRGKRRREKQRPT
DCHL*
UNDERLINED = PEP2A sequence
BOLD UNDERLINED = Matrix binding tag

In some embodiments, engineered VACV viruses of the present technology comprise an expression construct such as that illustrated by SEQ ID NO: 29 (Table 5) inserted into the VACV genomic region that corresponds to the position of the TK locus (e.g., position 80,962 to 81,032 of SEQ ID NO: 2).

### VACVΔB2R

The VACV B2R gene encodes poxin, a nuclease that plays a role in viral evasion of host cGAS-STING innate immunity. The disclosure of the present technology relates to a B2R mutant vaccinia virus (i.e., VACVΔB2R; VACV comprising a B2R deletion; VACV genetically engineered to comprise a mutant B2R gene), or immunogenic compositions comprising the virus, in which the virus is engineered to express one or more specific genes of interest (SG), such as OX40L (VACVΔB2R-OX40L), and its use as a cancer immunotherapeutic. In some embodiments, the B2R gene of the vaccinia virus, through homologous recombination techniques, is engineered to contain a disruption comprising a heterologous nucleic acid sequence comprising one or more expression cassettes, which result in a B2R knockout such that the B2R gene is not expressed, expressed at levels so low as to have no effect, or the expressed protein is non-functional (e.g., is a null-mutation). In some embodiments, the ΔB2R mutant includes a heterologous nucleic acid sequence in place of all or a majority of the B2R gene sequence. For example, in some embodiments, the nucleic acid sequence corresponding to the position of B2R in the VACV genome (e.g., position 164,856 to 165,530 of SEQ ID NO: 2) is replaced with a heterologous nucleic acid sequence. In some embodiments, the heterologous nucleic acid sequence comprises an open reading frame that encodes a selectable marker. In some embodiments, the selectable marker is a fluorescent protein (e.g., gpt, GFP, mCherry). In some embodiments, the VACVΔB2R virus encompasses a recombinant VACV that does not express a functional B2R protein. In some embodiments, a specific gene of interest (e.g., OX40L, hFlt3L) is inserted into the B2R locus of the VACV genome, splitting the B2R gene and obliterating it.

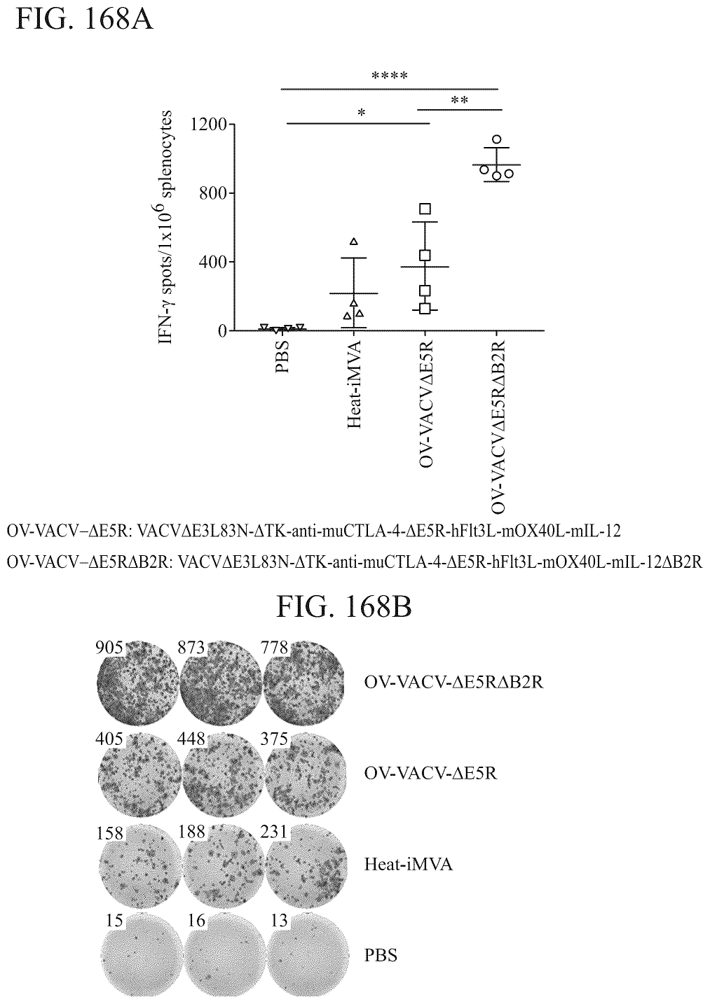

Additionally or alternatively, in some embodiments, VACVΔB2R is engineered to express both OX40L and hFlt3L. In some embodiments, the thymidine kinase (TK) gene of the vaccinia virus (e.g., position 80,962 to 81,032 of SEQ ID NO: 2), through homologous recombination, is engineered to contain a disruption comprising a heterologous nucleic acid sequence comprising one or more expression cassettes, which results in a TK gene knockout such that the TK gene is not expressed, expressed at levels so low as to have no effect, or the expressed protein is non-functional (e.g., is a null-mutation). The resulting VACVΔB2R-TK(−) virus is further engineered to comprise one or more expression cassettes that are flanked by a partial sequence of the TK gene (TK-L and TK-R) on either side. In some embodiments, the expression cassette comprises a single open reading frame that encodes a specific gene of interest (SG), such as OX40L using the vaccinia viral synthetic early and late promoter (PsE/L), resulting in VACVΔB2R-TK(−)-OX40L. In some embodiments, the recombinant virus is further modified at the B2R locus, through homologous recombination techniques, to contain a disruption comprising a heterologous nucleic acid sequence comprising one or more expression cassettes, which result in a B2R knockout such that the B2R gene is not expressed, expressed at levels so low as to have no effect, or the expressed protein is non-functional (e.g., is a null-mutation). In some embodiments, the expression cassette comprises a single open reading frame that encodes a specific gene of interest (SG), such as hFlt3L, resulting in VACVΔB2R-hFlt3L-TK(−)-OX40L. In some embodiments, the expression cassette encoding OX40L is inserted into the B2R locus while the expression cassette encoding hFlt3L is inserted into the TK locus. In some embodiments, a VACVΔB2R-anti-CTLA-4-hFlt3L-TK(−) virus is further modified to encode OX40L, resulting in VACVΔB2R-anti-CTLA-4-TK(−)-hFlt3L-OX40L. In some embodiments, the VACVΔB2R-anti-CTLA-4-TK(−)-hFlt3L-OX40L virus is further modified to express hIL-12, resulting in VACVΔB2R-anti-CTLA-4-TK(−)-hFlt3L-OX40L-hIL-12. In some embodiments, a VACVΔE3L83N-ΔTK-anti-CTLA-4-ΔE5R-hFlt3L-OX40L-hIL-12 genome is modified to comprise a B2R deletion (VACVΔE3L83N-ΔTK-anti-CTLA-4-ΔE5R-hFlt3L-OX40L-IL-12-ΔB2R) (see, e.g., FIG. 168).

Additionally or alternatively, in some embodiments, the heterologous nucleic acid sequence comprises an expression cassette comprising two or more open reading frames encoding two or more specific genes of interest, separated by a nucleotide sequence that encodes, in the 5′ to 3′ direction, a protease cleavage site (e.g., a furin cleavage site) and a 2A peptide (Pep2A) sequence.

In some embodiments, the genetically engineered or recombinant VACVΔB2R viruses described above are modified to express at least one heterologous gene, such as any one or more of hOX40L, hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or include at least one other viral gene mutation or deletion, such as any one or more of the following deletions: E3L (ΔE3L); E3LΔ83N; C7 (ΔC7L); B19R (B18R; ΔWR200); E5R (ΔE5R); K7R; C12L (IL18BP); B8R; B14R; N1L; C11R; K1L; M1L; N2L; and/or WR199. In some embodiments, the genetically engineered or recombinant VACVΔB2R viruses are selected from VACVΔB2R-ΔE5R, VACVΔB2R-ΔE5R-E3LΔ83N, and VACVΔB2R-E3LΔ83N. In other embodiments, no further heterologous genes are added other than those provided in the name of the virus herein, and/or no further viral genes are disrupted or deleted other than those provided in the name of the virus herein.

Although in certain embodiments described above, the transgene may be inserted into the B2R locus, splitting the B2R gene and obliterating it or replacing it, other suitable integration loci can be selected. For example, VACV encodes several immune modulatory genes, including but not limited to C7, C11, K3, F1, F2, F4, F6, F8, F9, F11, F14.5, J2, A46, E3L, B18R (WR200), E5R, K7R, C12L, B8R, B14R, N1L, C11R, K1L, C16, M1L, N2L, and WR199. Accordingly, in some embodiments, these genes can be deleted to potentially enhance immune activating properties of the virus and allow insertion of transgenes.

In some embodiments, the heterologous nucleotide sequence further comprises an additional expression cassette comprising an open reading frame that encodes a selectable marker operably linked to a promoter that is capable of directing expression of the selectable marker. In some embodiments, the selectable marker is a xanthine-guanine phosphoribosyl transferase (gpt) gene. In some embodiments, the selectable marker is a green fluorescent protein (GFP) gene. In some embodiments, the selectable marker is an mCherry gene encoding a red fluorescent protein.

A non-limiting example of a VACVΔB2R deletion construct comprising an open reading frame encoding a selectable marker according to the present technology is shown in SEQ ID NO: 30 (Table 7).

TABLE 7
Exemplary nucleotide sequences for the open reading frames of
the recombinant VACVΔB2R constructs of the present technology.

B2R-FRT GFP Kan plasmid nucleic acid sequence
(SEQ ID NO: 30)
GTCTATACAAATCCATTAATGTGGAATATCGATTCTTGGTAATTAATAGATTAG
GTGCAGATCTAGATGCGGTGATCAGAGCCAATAATAATAGATTACCAAAAAGG
TCGGTGATGTTGATCGGAATCGAAATCTTAAATACCATACAATTTATGCACGA
GCAAGGATATTCTCACGGAGATATTAAAGCGAGTAATATAGTCTTGGATCAAA
TAGATAAGAATAAATTATATCTAGTGGATTACGGATTGGTTTCTAAATTCATGT
CTAATGGAGAACATGTTCCATTTATAAGAAATCCAAATAAAATGGATAACGGT
ACTCTAGAATTTACACCTATAGATTCGCATAAAGGATACGTTGTATCTAGACGT
GGAGATCTAGAAACACTTGGATATTGTATGATTAGATGGTTGGGAGGTATCTT
GCCATGGACTAAGATATCTGAAACAAAGAATTGTGCATTAGTAAGTGCCACAA
AACAGAAATATGTTAACAATACTGCGACTTTGTTAATGACCAGTTTGCAATATG
CACCTAGAGAATTGCTGCAATATATTACCATGGTAAACTCTTTGACATATTTTG
AGGAACCCAATTATGACGAGTTTCGGCACATATTAATGCAGGGTGTATATTATT
AAGTGTGGTGTTTGGTCGATGTAAAATTTTTGTCGATAAAAATTAAAAAATAA
CTTAATTTATTATTGATCTCGTGTATAAGCTAGCGCGGTTAACCGCCTTTTTATC
CATCAGGTGATCTGTTTTTATTGTGGAGTCTAGAACTAGTGGATCCCCCGGGCT
GCAGGAATTCGATATCAAGCTCAGGCCTAGATCTGTCGACTTCGAGCTTATTTA
TATTCCAAAAAAAAAAAATAAAATTTCAATTTTTAAGCTTTTGAAGTTCCTATA
CTTTCTAGAGAATAGGAACTTCCACTAATTCCAAACCCACCCGCTTTTTATAGT
AAGTTTTTCACCCATAAATAATAAATACAATAATTAATTTCTCGTAAAAGTAGA
AAATATATTCTAATTTATTGCACGGTAAGGAAGTAGATCATAACTCGAGGAAT
TGGGGATCTCTATAATCTCGCGCAACCTATTTTCCCCTCGAACACTTTTTAAGC
CGTAGATAAACAGGCTGGGACACTTCACACGCGTATGGTGAGCAAGGGCGAG
GAGCTGTTCACCGGGGTGGTGCCCATCCTGGTCGAGCTGGACGGCGACGTAAA
CGGCCACAAGTTCAGCGTGTCCGGCGAGGGCGAGGGCGATGCCACCTACGGC
AAGCTGACCCTGAAGTTCATCTGCACCACCGGCAAGCTGCCCGTGCCCTGGCC
CACCCTCGTGACCACCCTGACCTACGGCGTGCAGTGCTTCAGCCGCTACCCCG
ACCACATGAAGCAGCACGACTTCTTCAAGTCCGCCATGCCCGAAGGCTACGTC
CAGGAGCGCACCATCTTCTTCAAGGACGACGGCAACTACAAGACCCGCGCCGA
GGTGAAGTTCGAGGGCGACACCCTGGTGAACCGCATCGAGCTGAAGGGCATC
GACTTCAAGGAGGACGGCAACATCCTGGGGCACAAGCTGGAGTACAACTACA
ACAGCCACAACGTCTATATCATGGCCGACAAGCAGAAGAACGGCATCAAGGT
GAACTTCAAGATCCGCCACAACATCGAGGACGGCAGCGTGCAGCTCGCCGACC
ACTACCAGCAGAACACCCCCATCGGCGACGGCCCCGTGCTGCTGCCCGACAAC
CACTACCTGAGCACCCAGTCCAAGCTGAGCAAAGACCCCAACGAGAAGCGCG
ATCACATGGTCCTGCTGGAGTTCGTGACCGCCGCCGGGATCACTCTCGGCATG
GACGAGCTGTACAAGTAATGAAGTTCCTATACTTTCTAGAGAATAGGAACTTC
CGGTTAACCGGTGATATCACCTCTCTGGAAGACAGCGTGAATAATGTACTCAT
GAAACGTTTGGAAACTATACGCCATATGTGGTCTGTCGTATATGATCATTTTGA
TATTGTGAATGGTAAAGAATGCTGTTATGTGCATACGCATTTGTCTAATCAAAA
TCTTATACCGAGTACTGTAAAAACAAATTTGTACATGAAGACTATGGGATCAT
GCATTCAAATGGATTCCATGGAAGCTCTAGAGTATCTTAGCGAACTGAAGGAA
TCAGGTGGATGGAGTCCCAGACCAGAAATGCAGGAATTTGAATATCCAGATGG
AGTGGAAGACACTGAATCAATTGAGAGATTGGTAGAGGAGTTCTTCAATAGAT
CAGAACTTCAGGCTGGTGAATCAGTCAAATTTGGTAATTCTATTAATGTTAAAC
ATACATCTGTTTCAGCTAAGCAACTAAGAACACGTATACGGCAGCAGCTTCCTT
TATACTCTCATCTTTTACCAACACAAAGGGTGGATATTTGTTCATTGGAGTTGA
TAATAATACACACAAAGTAATTGGATTCACGGTGGGTCATGACTACCTCAGAC
TGGTAGAGAATGATATAGAAAAGCATATCAAAAGACTTCGTGTTGTGCATTTC
TGTGAGAAGAAAGAGGACATCAAGTACACGTGTCGATTCATCAAGGTATATAA
ACCTGGGGATGAGGCTTCACGTAGAAAGCCAGTCCGCAGAAACGGTGCTGACC
CCGGATGAATGTCAGCTACTGGGCTATCTGGACAAGGGAAAACGCAAGCGCA
AAGAGAAAGCAGGTAGCTTGCAGTGGGCTTACATGGCGATAGCTAGACTGGGC
GGTTTTATGGACAGCAAGCGAACCGGAATTGCCAGCTGGGGCGCCCTCTGGTA
AGGTTGGGAAGCCCTGCAAAGTAAACTGGATGGCTTTCTCGCCGCCAAGGATC
TGATGGCGCAGGGGATCAAGCTCTGATCAAGAGACAGGATGAGGATCGTTTCG
CATGATTGAACAAGATGGATTGCACGCAGGTTCTCCGGCCGCTTGGGTGGAGA
GGCTATTCGGCTATGACTGGGCACAACAGACAATCGGCTGCTCTGATGCCGCC
GTGTTCCGGCTGTCAGCGCAGGGGCGCCCGGTTCTTTTTGTCAAGACCGACCTG
TCCGGTGCCCTGAATGAACTGCAAGACGAGGCAGCGCGGCTATCGTGGCTGGC
CACGACGGGCGTTCCTTGCGCAGCTGTGCTCGACGTTGTCACTGAAGCGGGAA
GGGACTGGCTGCTATTGGGCGAAGTGCCGGGGCAGGATCTCCTGTCATCTCAC
CTTGCTCCTGCCGAGAAAGTATCCATCATGGCTGATGCAATGCGGCGGCTGCA
TACGCTTGATCCGGCTACCTGCCCATTCGACCACCAAGCGAAACATCGCATCG
AGCGAGCACGTACTCGGATGGAAGCCGGTCTTGTCGATCAGGATGATCTGGAC
GAAGAGCATCAGGGGCTCGCGCCAGCCGAACTGTTCGCCAGGCTCAAGGCGA
GCATGCCCGACGGCGAGGATCTCGTCGTGACCCATGGCGATGCCTGCTTGCCG
AATATCATGGTGGAAAATGGCCGCTTTTCTGGATTCATCGACTGTGGCCGGCTG
GGTGTGGCGGACCGCTATCAGGACATAGCGTTGGCTACCCGTGATATTGCTGA
AGAGCTTGGCGGCGAATGGGCTGACCGCTTCCTCGTGCTTTACGGTATCGCCG
CTCCCGATTCGCAGCGCATCGCCTTCTATCGCCTTCTTGACGAGTTCTTCTGAA
TTATTAACGCTTACAATTTCCTGATGCGGTATTTTCTCCTTACGCATCTGTGCGG
TATTTCACACCGCATACAGGTGGCACTTTTCGGGGAAATGTGCGCGGAACCCC
TATTTTCGTTCCACTGAGCGTCAGACCCCGTAGAAAAGATCAAAGGATCTTCTT
GAGATCCTTTTTTTCTGCGCGTAATCTGCTGCTTGCAAACAAAAAAACCACCGC
TACCAGCGGTGGTTTGTTTGCCGGATCAAGAGCTACCAACTCTTTTTCCGAAGG
TAACTGGCTTCAGCAGAGCGCAGATACCAAATACTGTCCTTCTAGTGTAGCCGT
AGTTAGGCCACCACTTCAAGAACTCTGTAGCACCGCCTACATACCTCGCTCTGC
TAATCCTGTTACCAGTGGCTGCTGCCAGTGGCGATAAGTCGTGTCTTACCGGGT
TGGACTCAAGACGATAGTTACCGGATAAGGCGCAGCGGTCGGGCTGAACGGG
GGGTTCGTGCACACAGCCCAGCTTGGAGCGAACGACCTACACCGAACTGAGAT
ACCTACAGCGTGAGCTATGAGAAAGCGCCACGCTTCCCGAAGGGAGAAAGGC
GGACAGGTATCCGGTAAGCGGCAGGGTCGGAACAGGAGAGCGCACGAGGGAG
CTTCCAGGGGGAAACGCCTGGTATCTTTATAGTCCTGTCGGGTTTCGCCACCTC
TGACTTGAGCGTCGATTTTTGTGATGCTCGTCAGGGGGGCGGAGCCTATGGAA
AAACGCCAGCAACGCGGCCTTTTTACGGTTCCTGGGCTTTTGCTGGCCTTTTGC
TCACATGTTCTTTCCTGCGTTATCCCCTGATTCTGTGGATAACCGTATTACCGCC
TTTGAGTGAGCTGATACCGCTCGCCGCAGCCGAACGACCGAGCGCAGCGAGTC
A

In some embodiments, engineered VACVΔB2R virus is generated by inserting an expression construct such as that illustrated by SEQ ID NO: 30 (Table 7) into the VACV genomic region that corresponds to the position of the B2R locus (e.g., position 164,856 to 165,530 of SEQ ID NO: 2).

### MVAΔWR199

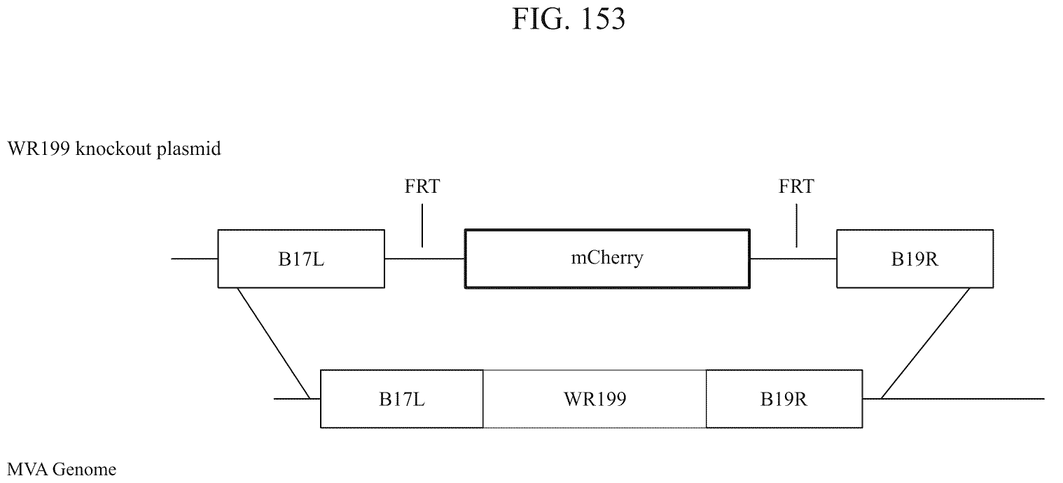

A non-limiting example of a MVAΔWR199 construct open reading frame according to the present technology is shown in SEQ ID NO: 34 (Table 8). In some embodiments, MVA comprising a ΔWR199 mutant is generated by inserting an expression construct, with, e.g., a WR199 knockout plasmid, such as that illustrated by SEQ ID NO: 34 into the MVA genomic region that corresponds to the position of the WR199 locus (e.g., position 158,399 to 160,143 of the sequence set forth in GenBank Accession No. AY603355) (see, e.g., FIG. 153).

TABLE 8
Exemplary nucleotide sequence for the open reading frames of the
recombinant MVAΔWR199 construct of the present technology.

WR199-FRT mCherry Kan plasmid nucleic acid sequence
(SEQ ID NO: 34)
CGTGAATGTATGTTGTTACATTTCCATGTCAATTGAGTTTATAAGAATTTTTATACATTATCTTCC
AACAAACAATTGACGAACGTATTGCTATGATTAACTCCCACGATACTATGCATATTATTAATCAT
TAACTTGCAGACTATACCTAGTGCTATTTTGACATACTCATGTTCTTGTGTAATTGCGGTATCTAT
ATTATTAAAGTACGTAAATCTAGCTATAGTTTTATTATTTAATTTTAGATAATATACCGTCTCCTT
ATTTTTAAAAATTGCCACATCCTTTATTAAATCATGAATGGGAATTTCTATGTCATCGTTAATAT
ATTGTGAACAACAAGAGCAGATATCTATAGGAAAGGGTGGAATGCGATACATTGATCTATGTA
GTTTTAAAACACACGCGAACTTTGAAGAATTTATATAAATCATTCCATCGATACATCCTTCTATG
TTGACATGTATATATCCAGGAATTCTTTTATTAATGTCAGGAAATGTATAAACTAAAACATTGCC
CGAAAGCGGTGCCTCTATCTGCGTTATATCCGTTCTTAACTTACAAAATGTAACCAATACCTTTG
CATGACTTGTTTTGTTCGGCAACGTTAGTTTAAACTTGACGAATGGATTAATTACAATAGCATGA
TCCGCGCATCTATTAAGTTTTTTTACTTTAACGCCCTTGTATGTTTTTACAGAGACTTTATCTAAA
TTTCTAGTGCTTGTATGTGTTATAAATATAACGGGATATAGAACTGAATCACCTACCTTAGATAC
CCAATTACATTTTATCAGATCCAGATAATAAACAAATTTTGTCGCCCTAACTAATTCTATATTGT
TATATATTTTACAATTGGTTATGATATCATGTAATAACTTGGAGTCTAACGCGCATCGTCGTACG
TTTATACAATTGTGATTTAGTGTAGTATATCTACACATGTATTTTTCCGCACTATAGTATTCTGGA
CTAGTGATAAAACTATCGTTATATCTGTCTTCAATGAACTCATCGAGATATTGCTCTCTGTCATA
TTCATACACCTGCATAAACTTTCTAGACATCTTACAATCCGTGTTATTTTAGGATCATATTTACAT
ATTTACGGGTATATCAAAGATGTTAGATTAGTTAATGGGAATCGTCTATAATAATGAATATTAA
ACAATTATATGAGGACTTTAAGCTAGCGCGGTTAACCGCCTTTTTATCCATCAGGTGATCTGTTT
TTATTGTGGAGTCTAGAACTAGTGGATCCCCCGGGCTGCAGGAATTCGATATCAAGCTCAGGCC
TAGATCTGTCGACTTCGAGCTTATTTATATTCCAAAAAAAAAAAATAAAATTTCAATTTTTAAGC
TTTTGAAGTTCCTATACTTTCTAGAGAATAGGAACTTCCACTAATTCCAAACCCACCCGCTTTTT
ATAGTAAGTTTTTCACCCATAAATAATAAATACAATAATTAATTTCTCGTAAAAGTAGAAAATA
TATTCTAATTTATTGCACGGTAAGGAAGTAGATCATAACTCGAGGAATTGGGGATCTCTATAAT
CTCGCGCAACCTATTTTCCCCTCGAACACTTTTTAAGCCGTAGATAAACAGGCTGGGACACTTCA
CACGCGTATGGTGAGCAAGGGCGAGGAGGATAACATGGCCATCATCAAGGAGTTCATGCGCTT
CAAGGTGCACATGGAGGGCTCCGTGAACGGCCACGAGTTCGAGATCGAGGGCGAGGGCGAGGG
CCGCCCCTACGAGGGCACCCAGACCGCCAAGCTGAAGGTGACCAAGGGTGGCCCCCTGCCCTTC
GCCTGGGACATCCTGTCCCCTCAGTTCATGTACGGCTCCAAGGCCTACGTGAAGCACCCCGCCG
ACATCCCCGACTACTTGAAGCTGTCCTTCCCCGAGGGCTTCAAGTGGGAGCGCGTGATGAACTT
CGAGGACGGCGGCGTGGTGACCGTGACCCAGGACTCCTCCCTGCAGGACGGCGAGTTCATCTAC
AAGGTGAAGCTGCGCGGCACCAACTTCCCCTCCGACGGCCCCGTAATGCAGAAGAAGACCATG
GGCTGGGAGGCCTCCTCCGAGCGGATGTACCCCGAGGACGGCGCCCTGAAGGGCGAGATCAAG
CAGAGGCTGAAGCTGAAGGACGGCGGCCACTACGACGCTGAGGTCAAGACCACCTACAAGGCC
AAGAAGCCCGTGCAGCTGCCCGGCGCCTACAACGTCAACATCAAGTTGGACATCACCTCCCACA
ACGAGGACTACACCATCGTGGAACAGTACGAACGCGCCGAGGGCCGCCACTCCACCGGCGGCA
TGGACGAGCTGTACAAGTAATGAAGTTCCTATACTTTCTAGAGAATAGGAACTTCCGGTTAACC
GGTGATTGTATTTTTCTCATGCGATGTGTGTAAAAAAACTGATATTATATAAATATTTTAGTGCC
GTATAATGAAGATGACGATGAAAATGATGGTACATATATATTTCGTATCATTATTGTTATTGCTA
TTCCACAGTTACGCCATAGACATCGAAAATGAAATCACAGAATTCTTCAATAAAATGAGAGATA
CTCTACCAGCTAAAGACTCTAAATGGTTGAATCCAGCATGTATGTTCGGAGGCACAATGAATGA
TATAGCCGCTCTAGGAGAGCCATTCAGCGCAAAGTGTCCTCCTATTGAAGACAGTCTTTTATCGC
ACAGATATAAAGACTATGTGGTTAAATGGGAGAGGCTAGAAAAAAATAGACGGCGACAGGTTT
CTAATAAACGTGTTAAACATGGTGATTTATGGATAGCCAACTATACATCTAAATTCAGTAACCG
TAGGTATTTGTGTACCGTAACTACAAAGAATGGTGACTGTGTTCAGGGTATAGTTAGATCTCAT
ATTAAAAAACCTCCTTCATGCATTCCAAAAACATATGAACTAGGTACTCATGATAAGTATGGCA
TAGACTTATACTGTGGAATTCTTTACGCAAAACATTATAATAATATAACTTGGTATAAAGATAAT
AAGGAAATTAATATCGACGATATTAAGTATTCACAAACGGGAAAGAAATTAATTATTCATAATC
CAGAGTTAGAAGATAGTGGAAGATACAACTGTTACGTTCATTACGACGACGTTAGAATCAAGAT
GTAAAATACTTACGGTTATACCGTCGCAAGACCACAGGTTTAAACTAATACTAGATCCAAAAAT
CAACGTAACGATAGGAGAACCTGCCAATATAACATGCACTGCTGTGTCAACGTCATTATTGATT
GACGATGTACTGATTGAATGGGAAAATCCATCCGGATGGCTTATAGGATTCGATTTTGATGTAT
ACTCTGTTTTAACTAGTAGAGGCGGTATCACCGAGGCGACCTTGTACTTTGAAAATGTTACTGA
AGAATATATAGGTAATACATATAAATGTCGTGGACACAACTATTATTTTGAAAAAACCCTACAA
CTACAGTAGTATTGGAGTCCTTTTCACGTAGAAAGCCAGTCCGCAGAAACGGTGCTGACCCCGG
ATGAATGTCAGCTACTGGGCTATCTGGACAAGGGAAAACGCAAGCGCAAAGAGAAAGCAGGTA
GCTTGCAGTGGGCTTACATGGCGATAGCTAGACTGGGCGGTTTTATGGACAGCAAGCGAACCGG
AATTGCCAGCTGGGGCGCCCTCTGGTAAGGTTGGGAAGCCCTGCAAAGTAAACTGGATGGCTTT
CTCGCCGCCAAGGATCTGATGGCGCAGGGGATCAAGCTCTGATCAAGAGACAGGATGAGGATC
GTTTCGCATGATTGAACAAGATGGATTGCACGCAGGTTCTCCGGCCGCTTGGGTGGAGAGGCTA
TTCGGCTATGACTGGGCACAACAGACAATCGGCTGCTCTGATGCCGCCGTGTTCCGGCTGTCAG
CGCAGGGGCGCCCGGTTCTTTTTGTCAAGACCGACCTGTCCGGTGCCCTGAATGAACTGCAAGA
CGAGGCAGCGCGGCTATCGTGGCTGGCCACGACGGGCGTTCCTTGCGCAGCTGTGCTCGACGTT
GTCACTGAAGCGGGAAGGGACTGGCTGCTATTGGGCGAAGTGCCGGGGCAGGATCTCCTGTCAT
CTCACCTTGCTCCTGCCGAGAAAGTATCCATCATGGCTGATGCAATGCGGCGGCTGCATACGCT
TGATCCGGCTACCTGCCCATTCGACCACCAAGCGAAACATCGCATCGAGCGAGCACGTACTCGG
ATGGAAGCCGGTCTTGTCGATCAGGATGATCTGGACGAAGAGCATCAGGGGCTCGCGCCAGCC
GAACTGTTCGCCAGGCTCAAGGCGAGCATGCCCGACGGCGAGGATCTCGTCGTGACCCATGGC
GATGCCTGCTTGCCGAATATCATGGTGGAAAATGGCCGCTTTTCTGGATTCATCGACTGTGGCCG
GCTGGGTGTGGCGGACCGCTATCAGGACATAGCGTTGGCTACCCGTGATATTGCTGAAGAGCTT
GGCGGCGAATGGGCTGACCGCTTCCTCGTGCTTTACGGTATCGCCGCTCCCGATTCGCAGCGCA
TCGCCTTCTATCGCCTTCTTGACGAGTTCTTCTGAATTATTAACGCTTACAATTTCCTGATGCGGT
ATTTTCTCCTTACGCATCTGTGCGGTATTTCACACCGCATACAGGTGGCACTTTTCGGGGAAATG
TGCGCGGAACCCCTATTTTCGTTCCACTGAGCGTCAGACCCCGTAGAAAAGATCAAAGGATCTT
CTTGAGATCCTTTTTTTCTGCGCGTAATCTGCTGCTTGCAAACAAAAAAACCACCGCTACCAGCG
GTGGTTTGTTTGCCGGATCAAGAGCTACCAACTCTTTTTCCGAAGGTAACTGGCTTCAGCAGAGC
GCAGATACCAAATACTGTCCTTCTAGTGTAGCCGTAGTTAGGCCACCACTTCAAGAACTCTGTA
GCACCGCCTACATACCTCGCTCTGCTAATCCTGTTACCAGTGGCTGCTGCCAGTGGCGATAAGTC
GTGTCTTACCGGGTTGGACTCAAGACGATAGTTACCGGATAAGGCGCAGCGGTCGGGCTGAACG
GGGGGTTCGTGCACACAGCCCAGCTTGGAGCGAACGACCTACACCGAACTGAGATACCTACAG
CGTGAGCTATGAGAAAGCGCCACGCTTCCCGAAGGGAGAAAGGCGGACAGGTATCCGGTAAGC
GGCAGGGTCGGAACAGGAGAGCGCACGAGGGAGCTTCCAGGGGGAAACGCCTGGTATCTTTAT
AGTCCTGTCGGGTTTCGCCACCTCTGACTTGAGCGTCGATTTTTGTGATGCTCGTCAGGGGGGCG
GAGCCTATGGAAAAACGCCAGCAACGCGGCCTTTTTACGGTTCCTGGGCTTTTGCTGGCCTTTTG
CTCACATGTTCTTTCCTGCGTTATCCCCTGATTCTGTGGATAACCGTATTACCGCCTTTGAGTGAG
CTGATACCGCTCGCCGCAGCCGAACGACCGAGCGCAGCGAGTCA

### MYXVΔM31R

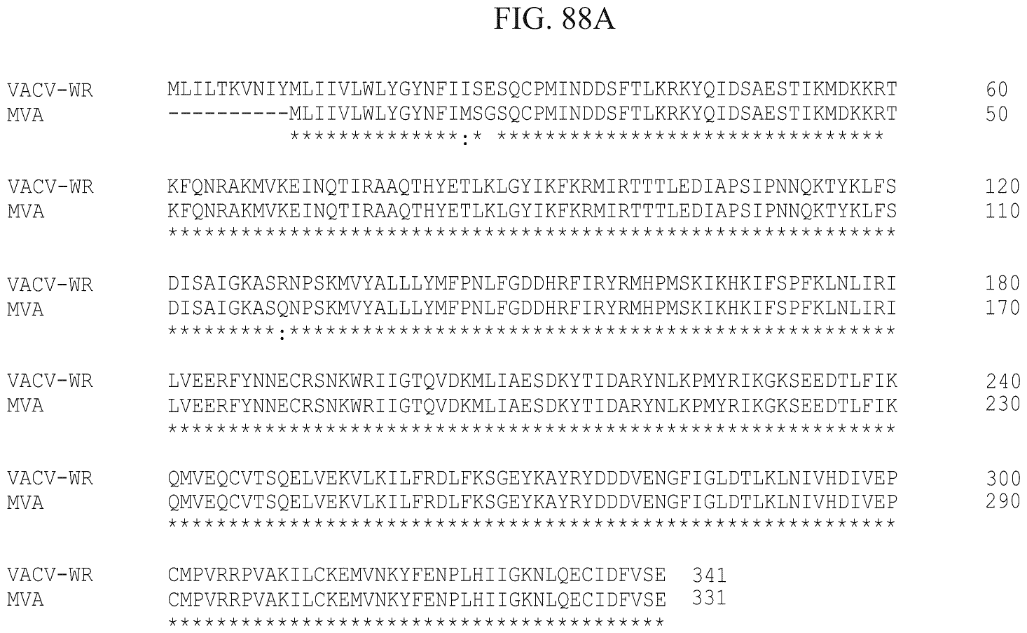

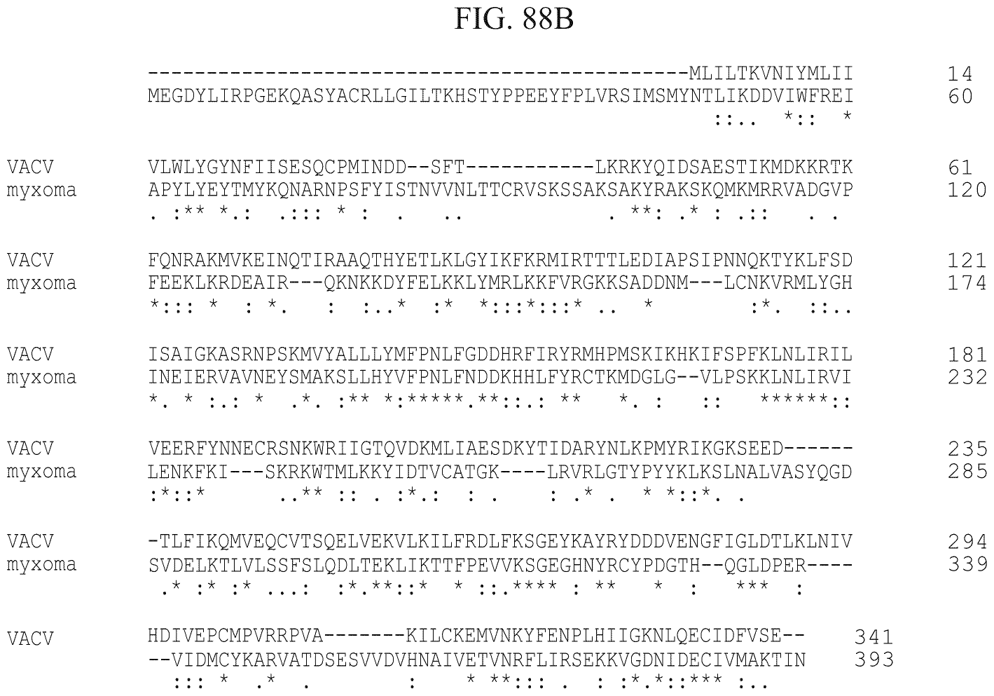

Myxoma M31R is orthologous to VACV E5R (see FIG. 88B). The disclosure of the present technology relates to a M31R mutant myxoma virus (i.e., MYXVΔM31R; MYXV comprising an M31R deletion; MYXV genetically engineered to comprise a mutant M31R gene), or immunogenic compositions comprising the virus, and its use as a cancer immunotherapeutic. In some embodiments, the MYXVΔM31R virus is engineered to express one or more specific genes of interest (SG), such as OX40L, for use as a cancer immunotherapeutic (MYXVΔM31R-OX40L). In some embodiments, the myxoma virus is derived from strain Lausanne (given by, e.g., GenBank Accession No. AF170726.2). In some embodiments, the M31R gene of the myxoma virus, through homologous recombination techniques, is engineered to contain a disruption comprising a heterologous nucleic acid sequence comprising one or more expression cassettes, which result in a M31R knockout such that the M31R gene is not expressed, expressed at levels so low as to have no effect, or the expressed protein is non-functional (e.g., is a null-mutation). In some embodiments, the ΔM31R mutant includes a heterologous nucleic acid sequence in place of all or a majority of the M31R gene sequence. For example, in some embodiments, the nucleic acid sequence corresponding to the position of M31R in the MYXV genome (e.g., position 30,138 to 31,319 of the myxoma genome) is replaced with a heterologous nucleic acid sequence comprising an open reading frame that encodes a specific gene of interest (SG), such as human OX40L, resulting in MYXVΔM31R-OX40L. In some embodiments, the expression cassette comprises a single open reading frame that encodes hFlt3L, resulting in MYXVΔM31R-hFlt3L.

Additionally or alternatively, in some embodiments, MYXVΔM31R is engineered to express both OX40L and hFlt3L. In some embodiments, the thymidine kinase (TK) gene of the myxoma virus (e.g., position 57,797 to 58,333 of the myxoma genome), through homologous recombination, is engineered to contain a disruption comprising a heterologous nucleic acid sequence comprising one or more expression cassettes, which results in a TK gene knockout such that the TK gene is not expressed, expressed at levels so low as to have no effect, or the expressed protein is non-functional (e.g., is a null-mutation). The resulting MYXVΔM31R-TK(−) virus is further engineered to comprise one or more expression cassettes that are flanked by a partial sequence of the TK gene (TK-L and TK-R) on either side. In some embodiments, the expression cassette comprises a single open reading frame that encodes a specific gene of interest (SG), such as OX40L using the vaccinia viral synthetic early and late promoter (PsE/L), resulting in MYXVΔM31R-TK(−)-OX40L. In some embodiments, the recombinant virus is further modified at the M31R locus, through homologous recombination techniques, to contain a disruption comprising a heterologous nucleic acid sequence comprising one or more expression cassettes, which result in a M31R knockout such that the M31R gene is not expressed, expressed at levels so low as to have no effect, or the expressed protein is non-functional (e.g., is a null-mutation). In some embodiments, the expression cassette comprises a single open reading frame that encodes a specific gene of interest (SG), such as hFlt3L, resulting in MYXVΔM31R-hFlt3L-TK(−)-OX40L. In some embodiments, the expression cassette encoding OX40L is inserted into the M31R locus while the expression cassette encoding hFlt3L is inserted into the TK locus. In some embodiments, a MYXVΔM31R-anti-CTLA-4-hFlt3L-TK(−) virus is further modified to encode OX40L, resulting in MYXVΔM31R-anti-CTLA-4-TK(−)-hFlt3L-OX40L. In some embodiments, the MYXVΔM31R-anti-CTLA-4-TK(−)-hFlt3L-OX40L virus is further modified to express hIL-12, resulting in MYXVΔM31R-anti-CTLA-4-TK(−)-hFlt3L-OX40L-hIL-12.

In some embodiments, the genetically engineered or recombinant MYXVΔM31R viruses described above are modified to express at least one heterologous gene, such as any one or more of hOX40L, hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or include at least one other viral gene mutation or deletion, such as any one or more of the following deletions: E3L (ΔE3L); E3LΔ83N; C7 (ΔC7L); B2R (ΔB2R), B19R (B18R; ΔWR200); E5R; K7R; C12L (IL18BP); B8R; B14R; N1L; C11R; K1L; M1L; N2L; and/or WR199. In other embodiments, no further heterologous genes are added other than those provided in the name of the virus herein (e.g., OX40L or OX40L and hFlt3L), and/or no further viral genes other than M31R or M31R and TK are disrupted or deleted.

Although in certain embodiments described above, the transgene may be inserted into the TK locus, splitting the TK gene and obliterating it, other suitable integration loci can be selected. For example, MYXV encodes several immune modulatory genes, including but not limited to C7, C11, K3, F1, F2, F4, F6, F8, F9, F11, F14.5, J2, A46, E3L, B18R (WR200), E5R, K7R, C12L, B8R, B14R, N1L, C11R, K1L, C16, M1L, N2L, and WR199.

Accordingly, in some embodiments, these genes can be deleted to potentially enhance immune activating properties of the virus and allow insertion of transgenes.

Additionally or alternatively, in some embodiments, the heterologous nucleic acid sequence comprises an expression cassette comprising two or more open reading frames encoding two or more specific genes of interest, separated by a nucleotide sequence that encodes, in the 5′ to 3′ direction, a protease cleavage site (e.g., a furin cleavage site) and a 2A peptide (Pep2A) sequence. For example, in some embodiments, MYXVΔM31R encompasses a recombinant MYXV in which all or a majority of the M31R gene sequence is replaced by a first specific gene of interest (e.g., hFtl3L) and a second specific gene of interest (e.g., OX40L), wherein the coding sequences of the first and second specific genes of interest are separated by a cassette including a furin cleavage site followed by a 2A peptide (Pep2A) sequence, thereby forming a recombinant virus such as MYXVΔM31R-hFlt3L-OX40L.

In some embodiments, the heterologous nucleotide sequence further comprises an additional expression cassette comprising an open reading frame that encodes a selectable marker operably linked to a promoter that is capable of directing expression of the selectable marker. In some embodiments, the selectable marker is a xanthine-guanine phosphoribosyl transferase (gpt) gene. In some embodiments, the selectable marker is a green fluorescent protein (GFP) gene. In some embodiments, the selectable marker is an mCherry gene encoding a red fluorescent protein.

### MYXVΔM63R and MYXVΔM64R

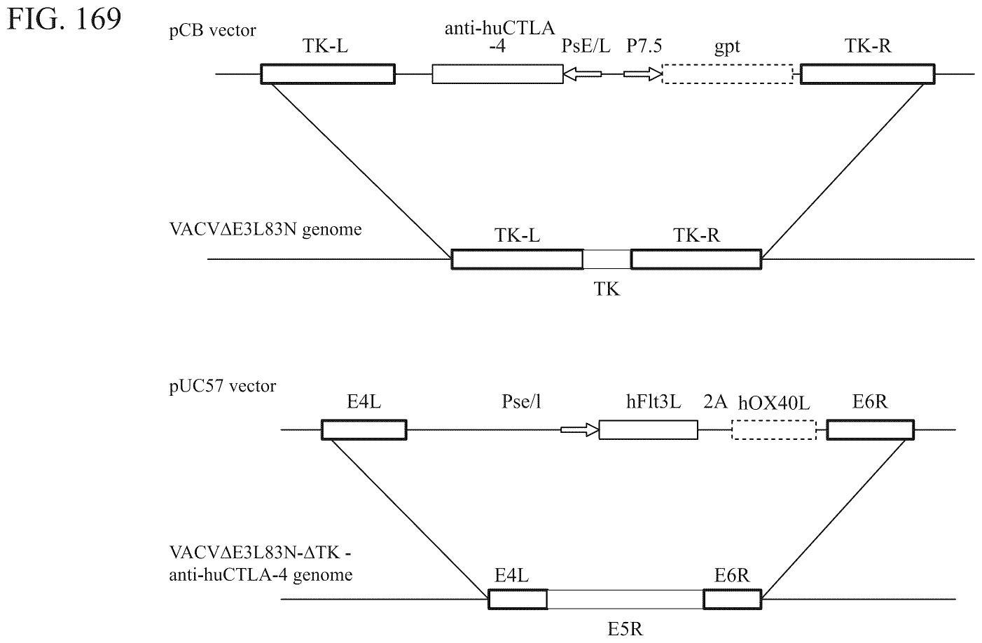

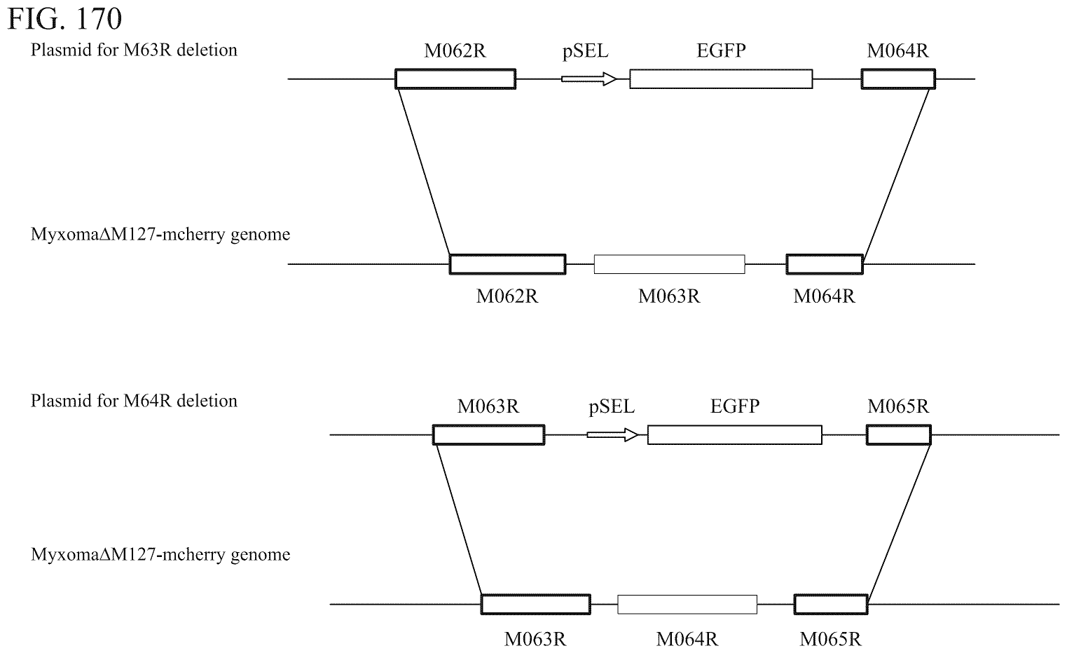

The disclosure of the present technology relates to an M63R mutant myxoma virus (i.e., MYXVΔM63R; MYXV comprising an M63R deletion; MYXV genetically engineered to comprise a mutant M63R gene), and an M64R mutant myxoma virus (i.e., MYXVΔM64R, MYXV comprising an M64R deletion; MYXV genetically engineered to comprise a mutant M64R gene), or immunogenic compositions comprising the viruses, and its use as a cancer immunotherapeutic. In some embodiments, the M63R or M64R mutants are inserted into a MYXVΔM127 mCherry genome (see, e.g., FIG. 170). In some embodiments, the MYXVΔM63R or MYXVΔM64R virus is engineered to express one or more specific genes of interest (SG), such as those disclosed herein, for use as a cancer immunotherapeutic. In some embodiments, the myxoma virus is derived from strain Lausanne (given by, e.g., GenBank Accession No. AF170726.2). In some embodiments, the M63R or M64R gene of the myxoma virus, through homologous recombination techniques, is engineered to contain a disruption comprising a heterologous nucleic acid sequence comprising one or more expression cassettes, which result in a M63R or M64R knockout such that the M63R or M64R gene is not expressed, expressed at levels so low as to have no effect, or the expressed protein is non-functional (e.g., is a null-mutation). In some embodiments, the ΔM63R or ΔM64R mutant includes a heterologous nucleic acid sequence (e.g., a heterologous nucleic acid sequence comprising an open reading frome that encodes a specific gene of interest (SG)).

In some embodiments, the genetically engineered or recombinant MYXVΔM63R or MYXVΔM64R viruses described above are modified to express at least one heterologous gene, such as any one or more of hOX40L, hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or include at least one other viral gene mutation or deletion, such as any one or more of the following deletions: E3L (ΔE3L); E3LΔ83N; C7 (ΔC7L); B2R (ΔB2R), B19R (B18R; ΔWR200); E5R; K7R; C12L (IL18BP); B8R; B14R; N1L; C11R; K1L; M1L; N2L; and/or WR199. In other embodiments, no further heterologous genes are added other than those provided in the name of the virus herein, and/or no further viral genes other than M63R or M64R are disrupted or deleted.

In some embodiments, the heterologous nucleotide sequence further comprises an additional expression cassette comprising an open reading frame that encodes a selectable marker operably linked to a promoter that is capable of directing expression of the selectable marker. In some embodiments, the selectable marker is a xanthine-guanine phosphoribosyl transferase (gpt) gene. In some embodiments, the selectable marker is a green fluorescent protein (GFP) gene. In some embodiments, the selectable marker is an mCherry gene encoding a red fluorescent protein.

Non-limiting examples of a MVAΔ63R and MVAΔ64R construct open reading frames according to the present technology are shown in SEQ ID NOs: 39 and 40 (Table 9).

TABLE 9
Exemplary nucleotide sequence for the open reading frames of the
recombinant MVAΔ63R and MVAΔ64R constructs of the present technology.

pUC57-dM63-GFP plasmid nucleic acid sequence
(SEQ ID NO: 40)
TCGCGCGTTTCGGTGATGACGGTGAAAACCTCTGACACATGCAGCTCCCGGAGACGGTCACAGC
TTGTCTGTAAGCGGATGCCGGGAGCAGACAAGCCCGTCAGGGCGCGTCAGCGGGTGTTGGCGG
GTGTCGGGGCTGGCTTAACTATGCGGCATCAGAGCAGATTGTACTGAGAGTGCACCATATGCGG
TGTGAAATACCGCACAGATGCGTAAGGAGAAAATACCGCATCAGGCGCCATTCGCCATTCAGG
CTGCGCAACTGTTGGGAAGGGCGATCGGTGCGGGCCTCTTCGCTATTACGCCAGCTGGCGAAAG
GGGGATGTGCTGCAAGGCGATTAAGTTGGGTAACGCCAGGGTTTTCCCAGTCACGACGTTGTAA
AACGACGGCCAGTGAATTCGAGCTCGGTACCCATCAAAATAAAATTGGATAAGGAAAAGACGT
TCAAATTCGTCATCGTATTGGAACCGGAGTGGATAGATGAGATAAAACCTATATACATGAAAGT
TAACGACGAGTCGGTGGAGTTAGAATTAGACTATAAAGACGCCATCAAACGCATCTATTCGGCG
GAGGTGGTATTATGTTCAGATTCCGTGATCAACCTGTTCAGTGACGTCGACGTGTCTTATACGTG
CGAATACCCTACGATTAAGGTGAATACGATAAAAAAATACTACAGCGTACAGAACAGAGGGAT
GACCTACGTACATATAGAATCGCCCATTAATACGAAAGATAAATGCTGGTTCGTGGAAAAGAAC
GGATGGTACGAGGATAGAACACATTCGTAATTTTTTTATATAGTGAAAAATAATGTGAGCATTA
CGAGCGTGGGTTTATCTACACGGCTAGCAAAAATTGAAATTTTATTTTTTTTTTTTGGAATATAA
ATAAGCTCGAAGTCGACAGATCTAGGCCTGGTACCATGGTGAGCAAGGGCGAGGAGCTGTTCA
CCGGGGTGGTGCCCATCCTGGTCGAGCTGGACGGCGACGTAAACGGCCACAAGTTCAGCGTGTC
CGGCGAGGGCGAGGGCGATGCCACCTACGGCAAGCTGACCCTGAAGTTCATCTGCACCACCGG
CAAGCTGCCCGTGCCCTGGCCCACCCTCGTGACCACCCTGACCTACGGCGTGCAGTGCTTCAGC
CGCTACCCCGACCACATGAAGCAGCACGACTTCTTCAAGTCCGCCATGCCCGAAGGCTACGTCC
AGGAGCGCACCATCTTCTTCAAGGACGACGGCAACTACAAGACCCGCGCCGAGGTGAAGTTCG
AGGGCGACACCCTGGTGAACCGCATCGAGCTGAAGGGCATCGACTTCAAGGAGGACGGCAACA
TCCTGGGGCACAAGCTGGAGTACAACTACAACAGCCACAACGTCTATATCATGGCCGACAAGC
AGAAGAACGGCATCAAGGTGAACTTCAAGATCCGCCACAACATCGAGGACGGCAGCGTGCAGC
TCGCCGACCACTACCAGCAGAACACCCCCATCGGCGACGGCCCCGTGCTGCTGCCCGACAACCA
CTACCTGAGCACCCAGTCCGCCCTGAGCAAAGACCCCAACGAGAAGCGCGATCACATGGTCCT
GCTGGAGTTCGTGACCGCCGCCGGGATCACTCTCGGCATGGACGAGCTGTACAAGTAAAGCTAG
CGATCAGGCCTTTTTTTTTATTGAAAAATAATAGTAAGAAAACGTTGCCGTAAACATGGAGGAG
GGTATCGTGCATAAATTAGACGTGTTTCTCATCGACGAAAACGTGTCTATAAAACACGTTAATTT
GTTCGACGGGGATAGTTACGGGTGCAACATCCATTTAAAGACCGCCACGTGTAAGTACATCACC
TTTATATTAGTCTTAGAACCCGATTGGGAGAACATAGTCGAGGCAAAACCCATTCACATGAGAT
TGAACGGCAAAAAGATACGCGTACCACTCGTAGCAAAAACCCACACGTCACTTATTTATAAAGT
CGTTATCTACGTGGAGGAAGACGCCCTCGCACGATTCTACAGCGACGTGGAAAGGTCGTACACG
GACGTGTATCCCACGTTTCTAGTCAATACGGATACGCGACGTTATTACATTTTGGATAGCGGAC
GGACGTATACGTACATAGATCCGTTTATATCGGACGAAGCTTGGCGTAATCATGGTCATAGCTG
TTTCCTGTGTGAAATTGTTATCCGCTCACAATTCCACACAACATACGAGCCGGAAGCATAAAGT
GTAAAGCCTGGGGTGCCTAATGAGTGAGCTAACTCACATTAATTGCGTTGCGCTCACTGCCCGC
TTTCCAGTCGGGAAACCTGTCGTGCCAGCTGCATTAATGAATCGGCCAACGCGCGGGGAGAGGC
GGTTTGCGTATTGGGCGCTCTTCCGCTTCCTCGCTCACTGACTCGCTGCGCTCGGTCGTTCGGCT
GCGGCGAGCGGTATCAGCTCACTCAAAGGCGGTAATACGGTTATCCACAGAATCAGGGGATAA
CGCAGGAAAGAACATGTGAGCAAAAGGCCAGCAAAAGGCCAGGAACCGTAAAAAGGCCGCGT
TGCTGGCGTTTTTCCATAGGCTCCGCCCCCCTGACGAGCATCACAAAAATCGACGCTCAAGTCA
GAGGTGGCGAAACCCGACAGGACTATAAAGATACCAGGCGTTTCCCCCTGGAAGCTCCCTCGTG
CGCTCTCCTGTTCCGACCCTGCCGCTTACCGGATACCTGTCCGCCTTTCTCCCTTCGGGAAGCGT
GGCGCTTTCTCATAGCTCACGCTGTAGGTATCTCAGTTCGGTGTAGGTCGTTCGCTCCAAGCTGG
GCTGTGTGCACGAACCCCCCGTTCAGCCCGACCGCTGCGCCTTATCCGGTAACTATCGTCTTGAG
TCCAACCCGGTAAGACACGACTTATCGCCACTGGCAGCAGCCACTGGTAACAGGATTAGCAGA
GCGAGGTATGTAGGCGGTGCTACAGAGTTCTTGAAGTGGTGGCCTAACTACGGCTACACTAGAA
GAACAGTATTTGGTATCTGCGCTCTGCTGAAGCCAGTTACCTTCGGAAAAAGAGTTGGTAGCTC
TTGATCCGGCAAACAAACCACCGCTGGTAGCGGTGGTTTTTTTGTTTGCAAGCAGCAGATTACG
CGCAGAAAAAAAGGATCTCAAGAAGATCCTTTGATCTTTTCTACGGGGTCTGACGCTCAGTGGA
ACGAAAACTCACGTTAAGGGATTTTGGTCATGAGATTATCAAAAAGGATCTTCACCTAGATCCT
TTTAAATTAAAAATGAAGTTTTAAATCAATCTAAAGTATATATGAGTAAACTTGGTCTGACAGTT
ACCAATGCTTAATCAGTGAGGCACCTATCTCAGCGATCTGTCTATTTCGTTCATCCATAGTTGCC
TGACTCCCCGTCGTGTAGATAACTACGATACGGGAGGGCTTACCATCTGGCCCCAGTGCTGCAA
TGATACCGCGAGACCCACGCTCACCGGCTCCAGATTTATCAGCAATAAACCAGCCAGCCGGAA
GGGCCGAGCGCAGAAGTGGTCCTGCAACTTTATCCGCCTCCATCCAGTCTATTAATTGTTGCCGG
GAAGCTAGAGTAAGTAGTTCGCCAGTTAATAGTTTGCGCAACGTTGTTGCCATTGCTACAGGCA
TCGTGGTGTCACGCTCGTCGTTTGGTATGGCTTCATTCAGCTCCGGTTCCCAACGATCAAGGCGA
GTTACATGATCCCCCATGTTGTGCAAAAAAGCGGTTAGCTCCTTCGGTCCTCCGATCGTTGTCAG
AAGTAAGTTGGCCGCAGTGTTATCACTCATGGTTATGGCAGCACTGCATAATTCTCTTACTGTCA
TGCCATCCGTAAGATGCTTTTCTGTGACTGGTGAGTACTCAACCAAGTCATTCTGAGAATAGTGT
ATGCGGCGACCGAGTTGCTCTTGCCCGGCGTCAATACGGGATAATACCGCGCCACATAGCAGAA
CTTTAAAAGTGCTCATCATTGGAAAACGTTCTTCGGGGCGAAAACTCTCAAGGATCTTACCGCT
GTTGAGATCCAGTTCGATGTAACCCACTCGTGCACCCAACTGATCTTCAGCATCTTTTACTTTCA
CCAGCGTTTCTGGGTGAGCAAAAACAGGAAGGCAAAATGCCGCAAAAAAGGGAATAAGGGCG
ACACGGAAATGTTGAATACTCATACTCTTCCTTTTTCAATATTATTGAAGCATTTATCAGGGTTA
TTGTCTCATGAGCGGATACATATTTGAATGTATTTAGAAAAATAAACAAATAGGGGTTCCGCGC
ACATTTCCCCGAAAAGTGCCACCTGACGTCTAAGAAACCATTATTATCATGACATTAACCTATA
AAAATAGGCGTATCACGAGGCCCTTTCGTC
pUC57-dM64-GFP plasmid nucleic acid sequence
(SEQ ID NO: 41)
TCGCGCGTTTCGGTGATGACGGTGAAAACCTCTGACACATGCAGCTCCCGGAGACGGTCACAGC
TTGTCTGTAAGCGGATGCCGGGAGCAGACAAGCCCGTCAGGGCGCGTCAGCGGGTGTTGGCGG
GTGTCGGGGCTGGCTTAACTATGCGGCATCAGAGCAGATTGTACTGAGAGTGCACCATATGCGG
TGTGAAATACCGCACAGATGCGTAAGGAGAAAATACCGCATCAGGCGCCATTCGCCATTCAGG
CTGCGCAACTGTTGGGAAGGGCGATCGGTGCGGGCCTCTTCGCTATTACGCCAGCTGGCGAAAG
GGGGATGTGCTGCAAGGCGATTAAGTTGGGTAACGCCAGGGTTTTCCCAGTCACGACGTTGTAA
AACGACGGCCAGTGAATTCGAGCTCGGTACCATGGAACTGATAAAATCTTTACACACGTCCACG
GACTTAACGGTCTACAGAACATCAATGCTCCATCACCGTAATATGCCCGAGAAGGAGTACTGCT
TCACGCAGATATACTCGGCTACGTTAAACATAGACACCAAGTCGACCGTCAGTTTTCGTAGTAC
GATACACGACGGGTTTCTTTCGACGTATCCGACGATCTACATTAATCCGGAGGAAAAATATTAC
AAAGTCCAGAACAAAGGACGTCTGCGGATGCGGGTGGTTACACCTATCTTAAACAGCGACAAA
CTACAGTTCATGGATAAGGGCGAGATGTATGCGGGTGTCGGCGACGACCCATCGATCGTAGACA
GTAGCGATAGCGACGATTATACCAGCAGTGAGGAAGACACGGAGGAGGAAGACACGGAGGAG
GAAGAAGATTGATTTTTTTTATTGAAAAATAATAGTAAGAAAACGTTGCCGTAAACGCTAGCAA
AAATTGAAATTTTATTTTTTTTTTTTGGAATATAAATAAGCTCGAAGTCGACAGATCTAGGCCTG
GTACCATGGTGAGCAAGGGCGAGGAGCTGTTCACCGGGGTGGTGCCCATCCTGGTCGAGCTGG
ACGGCGACGTAAACGGCCACAAGTTCAGCGTGTCCGGCGAGGGCGAGGGCGATGCCACCTACG
GCAAGCTGACCCTGAAGTTCATCTGCACCACCGGCAAGCTGCCCGTGCCCTGGCCCACCCTCGT
GACCACCCTGACCTACGGCGTGCAGTGCTTCAGCCGCTACCCCGACCACATGAAGCAGCACGAC
TTCTTCAAGTCCGCCATGCCCGAAGGCTACGTCCAGGAGCGCACCATCTTCTTCAAGGACGACG
GCAACTACAAGACCCGCGCCGAGGTGAAGTTCGAGGGCGACACCCTGGTGAACCGCATCGAGC
TGAAGGGCATCGACTTCAAGGAGGACGGCAACATCCTGGGGCACAAGCTGGAGTACAACTACA
ACAGCCACAACGTCTATATCATGGCCGACAAGCAGAAGAACGGCATCAAGGTGAACTTCAAGA
TCCGCCACAACATCGAGGACGGCAGCGTGCAGCTCGCCGACCACTACCAGCAGAACACCCCCA
TCGGCGACGGCCCCGTGCTGCTGCCCGACAACCACTACCTGAGCACCCAGTCCGCCCTGAGCAA
AGACCCCAACGAGAAGCGCGATCACATGGTCCTGCTGGAGTTCGTGACCGCCGCCGGGATCACT
CTCGGCATGGACGAGCTGTACAAGTAAAGCTAGCGATCAGGCCTGTTTTTATTATAATGATTTTT
AAATTTAAACGTTATTAAAAATGGAACCGGTATCTATGGACAAACCCTTTATGTACTTCGATGA
AATAGACGATGAATTAGAGTATGAACCCGAAAGCGTTAACGAAACACCTAAAAAACTCCCCCA
CCAGGGGCAGTTGAAATTATTGCTAGGCGAGTTGTTTTTTCTAAGTAAATTACAACGCCACGGC
ATTTTAGACGGATCCACGATCGTGTATATAGGATCCGCTCCCGGCACCCACATCAAATACTTAC
GCGATCATTTTATGTCTATGGGATTGGTTATTAAATGGATGTTGATCGACGGACGCACGCACGA
TCCCATCCTTGAAGGATTACGCGACGTAATTCTCATTACCAAGTTCGTCGACGAGGCGTACATTC
GACAGTTGAAGAAACAACTGTATCCGTCTAGGGTTATTCTCATTTCGGACGTGCGCTCGAAACG
GGGACAGAAAAGCTTGGCGTAATCATGGTCATAGCTGTTTCCTGTGTGAAATTGTTATCCGCTC
ACAATTCCACACAACATACGAGCCGGAAGCATAAAGTGTAAAGCCTGGGGTGCCTAATGAGTG
AGCTAACTCACATTAATTGCGTTGCGCTCACTGCCCGCTTTCCAGTCGGGAAACCTGTCGTGCCA
GCTGCATTAATGAATCGGCCAACGCGCGGGGAGAGGCGGTTTGCGTATTGGGCGCTCTTCCGCT
TCCTCGCTCACTGACTCGCTGCGCTCGGTCGTTCGGCTGCGGCGAGCGGTATCAGCTCACTCAA
AGGCGGTAATACGGTTATCCACAGAATCAGGGGATAACGCAGGAAAGAACATGTGAGCAAAAG
GCCAGCAAAAGGCCAGGAACCGTAAAAAGGCCGCGTTGCTGGCGTTTTTCCATAGGCTCCGCCC
CCCTGACGAGCATCACAAAAATCGACGCTCAAGTCAGAGGTGGCGAAACCCGACAGGACTATA
AAGATACCAGGCGTTTCCCCCTGGAAGCTCCCTCGTGCGCTCTCCTGTTCCGACCCTGCCGCTTA
CCGGATACCTGTCCGCCTTTCTCCCTTCGGGAAGCGTGGCGCTTTCTCATAGCTCACGCTGTAGG
TATCTCAGTTCGGTGTAGGTCGTTCGCTCCAAGCTGGGCTGTGTGCACGAACCCCCCGTTCAGCC
CGACCGCTGCGCCTTATCCGGTAACTATCGTCTTGAGTCCAACCCGGTAAGACACGACTTATCG
CCACTGGCAGCAGCCACTGGTAACAGGATTAGCAGAGCGAGGTATGTAGGCGGTGCTACAGAG
TTCTTGAAGTGGTGGCCTAACTACGGCTACACTAGAAGAACAGTATTTGGTATCTGCGCTCTGCT
GAAGCCAGTTACCTTCGGAAAAAGAGTTGGTAGCTCTTGATCCGGCAAACAAACCACCGCTGGT
AGCGGTGGTTTTTTTGTTTGCAAGCAGCAGATTACGCGCAGAAAAAAAGGATCTCAAGAAGATC
CTTTGATCTTTTCTACGGGGTCTGACGCTCAGTGGAACGAAAACTCACGTTAAGGGATTTTGGTC
ATGAGATTATCAAAAAGGATCTTCACCTAGATCCTTTTAAATTAAAAATGAAGTTTTAAATCAA
TCTAAAGTATATATGAGTAAACTTGGTCTGACAGTTACCAATGCTTAATCAGTGAGGCACCTAT
CTCAGCGATCTGTCTATTTCGTTCATCCATAGTTGCCTGACTCCCCGTCGTGTAGATAACTACGA
TACGGGAGGGCTTACCATCTGGCCCCAGTGCTGCAATGATACCGCGAGACCCACGCTCACCGGC
TCCAGATTTATCAGCAATAAACCAGCCAGCCGGAAGGGCCGAGCGCAGAAGTGGTCCTGCAAC
TTTATCCGCCTCCATCCAGTCTATTAATTGTTGCCGGGAAGCTAGAGTAAGTAGTTCGCCAGTTA
ATAGTTTGCGCAACGTTGTTGCCATTGCTACAGGCATCGTGGTGTCACGCTCGTCGTTTGGTATG
GCTTCATTCAGCTCCGGTTCCCAACGATCAAGGCGAGTTACATGATCCCCCATGTTGTGCAAAA
AAGCGGTTAGCTCCTTCGGTCCTCCGATCGTTGTCAGAAGTAAGTTGGCCGCAGTGTTATCACTC
ATGGTTATGGCAGCACTGCATAATTCTCTTACTGTCATGCCATCCGTAAGATGCTTTTCTGTGAC
TGGTGAGTACTCAACCAAGTCATTCTGAGAATAGTGTATGCGGCGACCGAGTTGCTCTTGCCCG
GCGTCAATACGGGATAATACCGCGCCACATAGCAGAACTTTAAAAGTGCTCATCATTGGAAAAC
GTTCTTCGGGGCGAAAACTCTCAAGGATCTTACCGCTGTTGAGATCCAGTTCGATGTAACCCACT
CGTGCACCCAACTGATCTTCAGCATCTTTTACTTTCACCAGCGTTTCTGGGTGAGCAAAAACAGG
AAGGCAAAATGCCGCAAAAAAGGGAATAAGGGCGACACGGAAATGTTGAATACTCATACTCTT
CCTTTTTCAATATTATTGAAGCATTTATCAGGGTTATTGTCTCATGAGCGGATACATATTTGAAT
GTATTTAGAAAAATAAACAAATAGGGGTTCCGCGCACATTTCCCCGAAAAGTGCCACCTGACGT
CTAAGAAACCATTATTATCATGACATTAACCTATAAAAATAGGCGTATCACGAGGCCCTTTCGT
C

### XI. Melanoma

Melanoma, one of the deadliest cancers, is the fastest growing cancer in the U.S. and worldwide. In most cases, advanced melanoma is resistant to conventional therapies, including chemotherapy and radiation. As a result, people with metastatic melanoma have a very poor prognosis, with a life expectancy of only 6 to 10 months. The discovery that about 50% of melanomas have mutations in BRAF (a key tumor-promoting gene) opened the door for targeted therapy of this disease. Early clinical trials with BRAF inhibitors showed remarkable, but unfortunately not sustainable, responses in patients with melanomas with BRAF mutations. Therefore, alternative treatment strategies for these patients, as well as others with melanoma without BRAF mutations, are urgently needed.

Human pathological data indicate that the presence of T-cell infiltrates within melanoma lesions correlates positively with longer patient survival (Oble et al., Cancer Immun. 9:3 (2009)). The importance of the immune system in protection against melanoma is further supported by partial success of immunotherapies, such as the immune activators IFN-α2b and IL-2 (Lacy et al., Expert Rev. Dermatol. 7(1):51-68 (2012)) as well as the unprecedented clinical responses of patients with metastatic melanoma to immune checkpoint therapy, including anti-CTLA-4 and anti-PD-1/PD-L1 as an agent alone or in combination therapy (Sharma & Allison, Science 348(6230)” 56-61 (2015); Hodi et al., NEJM 363(8):711-723 (2010); Wolchok et al., Lancet Oncol. 11(6):155-164 (2010); Topalian et al., NEJM 366(26):2443-2454 (2012); Wolchok et al., NEJM 369(2): 122-133 (2013); Hamid et al., NEJM 369(2):134-144 (2013); Tumeh et al., Nature 515(7528):568-571 (2014)). However, many patients fail to respond to immune checkpoint blockade therapy alone.

### XII. Type I IFN and the Cytosolic DNA-Sensing Pathway in Tumor Immunity

Type I IFN plays important roles in host antitumor immunity (Fuertes et al., Trends Immunol. 34:67-73 (2013)). IFNAR1-deficient mice are more susceptible to developing tumors after implantation of tumor cells; spontaneous tumor-specific T-cell priming is also defective in IFNAR1-deficient mice (Diamond et al., J. Exp. Med. 208:1989-2003 (2011); Fuertes et al., J. Exp. Med. 208:2005-2016 (2011)). More recent studies have shown that the cytosolic DNA-sensing pathway is important in the innate immune sensing of tumor-derived DNA, which leads to the development of antitumor CD8⁺ T-cell immunity (Woo et al., Immunity 41:830-842 (2014)). This pathway also plays a role in radiation-induced antitumor immunity (Deng et al., Immunity 4: 843-852 (2014)). Although spontaneous anti-tumor T-cell responses can be detected in patients with cancers, cancers eventually overcome host antitumor immunity in most patients. Novel strategies to alter the tumor immune suppressive microenvironment would be beneficial for cancer therapy.

### XIII. Immune Response

In addition to induction of the immune response by up-regulation of particular immune system activities (such as antibody and/or cytokine production, or activation of cell mediated immunity), immune responses may also include suppression, attenuation, or any other down-regulation of detectable immunity, so as to reestablish homeostasis and prevent excessive damage to the host's own organs and tissues. In some embodiments, an immune response that is induced according to the methods of the present disclosure generates effector T-cells (e.g., helper, killer, regulatory T-cells). In some embodiments, an immune response that is induced according to the methods of the present disclosure generates effector CD8⁺ (antitumor cytotoxic CD8⁺) T-cells or activated T helper (TH) cells (e.g., effector CD4⁺ T-cells), or both that can bring about directly or indirectly the death, or loss of the ability to propagate, of a tumor cell.

Induction of an immune response by the compositions and methods of the present disclosure may be determined by detecting any of a variety of well-known immunological parameters (Takaoka et al., Cancer Sci. 94:405-11 (2003); Nagorsen et al., Crit. Rev. Immunol. 22:449-62 (2002)). Induction of an immune response may therefore be established by any of a number of well-known assays, including immunological assays. Such assays include, but need not be limited to, in vivo, ex vivo, or in vitro determination of soluble immunoglobulins or antibodies; soluble mediators such as cytokines, chemokines, hormones, growth factors and the like as well as other soluble small peptide, carbohydrate, nucleotide and/or lipid mediators; cellular activation state changes as determined by altered functional or structural properties of cells of the immune system, for example cell proliferation, altered motility, altered intracellular cation gradient or concentration (such as calcium); phosphorylation or dephosphorylation of cellular polypeptides; induction of specialized activities such as specific gene expression or cytolytic behavior; cellular differentiation by cells of the immune system, including altered surface antigen expression profiles, or the onset of apoptosis (programmed cell death); or any other criterion by which the presence of an immune response may be detected. For example, cell surface markers that distinguish immune cell types may be detected by specific antibodies that bind to CD4⁺, CD8⁺, or NK cells. Other markers and cellular components that can be detected include but are not limited to interferon γ (IFN-γ), tumor necrosis factor (TNF), IFN-α, IFN-0 (IFNB), IL-6, and CCL5.

Common methods for detecting the immune response include, but are not limited to, flow cytometry, ELISA, immunohistochemistry. Procedures for performing these and similar assays are widely known and may be found, for example in Letkovits (Immunology Methods Manual: The Comprehensive Sourcebook of Techniques, Current Protocols in Immunology, 1998).

### XIV. Pharmaceutical Compositions and Preparations of the Present Technology

Disclosed herein are pharmaceutical compositions comprising MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 that may contain a carrier or diluent, which can be a solvent or dispersion medium containing, for example, water, saline, Tris buffer, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be effected by various antibacterial and antifungal agents and preservatives, for example, parabens, chlorobutanol, phenol, sorbic acid, thimerosal, and the like. In some embodiments, isotonic agents, for example, sugars or sodium chloride, and buffering agents are included. Prolonged absorption of the injectable compositions can be brought about by the use in the compositions of agents delaying absorption, for example, aluminum monostearate and gelatin or carrier molecules. Other excipients may include wetting or emulsifying agents. In general, excipients suitable for injectable preparations can be included as apparent to those skilled in the art.

Pharmaceutical compositions and preparations comprising MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 may be manufactured by means of conventional mixing, dissolving, granulating, emulsifying, encapsulating, entrapping or lyophilizing processes. Pharmaceutical viral compositions may be formulated in conventional manner using one or more physiologically acceptable carriers, diluents, excipients or auxiliaries that facilitate formulating virus preparations suitable for in vitro, in vivo, or ex vivo use. The compositions can be combined with one or more additional biologically active agents and may be formulated with a pharmaceutically acceptable carrier, diluent or excipient to generate pharmaceutical (including biologic) or veterinary compositions of the instant disclosure suitable for parenteral or intratumoral administration.

Many types of formulation are possible as is appreciated by those skilled in the art. The particular type chosen is dependent upon the route of administration chosen, as is well-recognized in the art. For example, systemic formulations will generally be designed for administration by injection, e.g., intravenous, as well as those designed for intratumoral delivery. In some embodiments, the systemic or intratumoral formulation is sterile.

Sterile injectable solutions are prepared by incorporating MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 in the required amount of the appropriate solvent with various other ingredients enumerated herein, as required, followed by suitable sterilization means. Generally, dispersions are prepared by incorporating the various sterilized active ingredients into a sterile vehicle that contains the basic dispersion medium and the required other ingredients from those enumerated above. In the case of sterile powders for the preparation of sterile injectable solutions, the preferred methods of preparation are vacuum drying and freeze-drying techniques, which yield a powder of the virus plus any additional desired ingredient from a previously sterile-filtered solution thereof.

In some embodiments, the MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 compositions of the present disclosure may be formulated in aqueous solutions, or in physiologically compatible solutions or buffers such as Hanks's solution, Ringer's solution, mannitol solutions or physiological saline buffer. In certain embodiments, any of the MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 compositions may contain formulator agents, such as suspending, stabilizing, penetrating or dispersing agents, buffers, lyoprotectants or preservatives such as polyethylene glycol, polysorbate 80, 1-dodecylhexahydro-2H-azepin-2-one (laurocapran), oleic acid, sodium citrate, Tris HCl, dextrose, propylene glycol, mannitol, polysorbate polyethylenesorbitan monolaurate (Tween®-20), isopropyl myristate, benzyl alcohol, isopropyl alcohol, ethanol sucrose, trehalose and other such generally known in the art may be used in any of the compositions of the instant disclosure. (Pramanick et al., Pharma Times 45(3):65-76 (2013)).

The biologic or pharmaceutical compositions of the present disclosure can be formulated to allow the virus contained therein to be available to infect tumor cells upon administration of the composition to a subject. The level of virus in serum, tumors, and if desired other tissues after administration can be monitored by various well-established techniques, such as antibody-based assays (e.g., ELISA, immunohistochemistry, etc.).

The engineered poxviruses of the present technology can be stored at −80° C. with a titer of 3.5×10⁷ pfu/mL formulated in about 10 mM Tris, 140 mM NaCl pH 7.7. For the preparation of vaccine shots, e.g., 10²-10⁸ or 10²-10⁹ viral particles can be lyophilized in 100 mL of phosphate-buffered saline (PBS) in the presence of 2% peptone and 1% human albumin in an ampoule, preferably a glass ampoule. Alternatively, the injectable preparations can be produced by stepwise freeze-drying of the engineered poxvirus in a formulation. This formulation can contain additional additives such as mannitol, dextran, sugar, glycine, lactose, or polyvinylpyrrolidone or other additives such as antioxidants or inert gas, stabilizers, or recombinant proteins (e.g., human serum albumin) suitable for in vivo administration. The glass ampoule is then sealed and can be stored between 4° C. and room temperature for several months. In some embodiments, the ampoule is stored at temperatures below −20° C.

For therapy, the lyophilisate can be dissolved in an aqueous solution, such as physiological saline or Tris buffer, and administered either systemically or intratumorally. The mode of administration, the dose, and the number of administrations can be optimized by those skilled in the art.

The pharmaceutical composition according to the present disclosure may comprise an additional adjuvant. As used herein, an “adjuvant” refers to a substance that enhances, augments, or potentiates the host's immune response to tumor antigens. A typical adjuvant may be aluminum salts, such as aluminum hydroxide or aluminum phosphate, Quil A, bacterial cell wall peptidoglycans, virus-like particles, polysaccharides, toll-like receptors, nano-beads, etc. (Aguilar et al., Vaccine 25:3752-3762 (2007)).

### XV. Therapeutic Methods of the Present Technology

In one aspect, the present disclosure provides a method for treating a solid tumor in a subject in need thereof, the method comprising administering to the subject an effective amount of: a recombinant modified vaccinia Ankara (MVA) virus comprising a deletion of E3L (MVAΔE3L) genetically engineered to express OX40L (MVAΔE3L-OX40L); a recombinant MVA virus comprising a deletion of C7L (MVAΔC7L) genetically engineered to express OX40L (MVAΔC7L-OX40L); a recombinant MVAΔC7L engineered to express OX40L and hFlt3L (MVAΔC7L-hFlt3L-OX40L); a recombinant MVA genetically engineered to comprise a deletion of C7L, a deletion of E5R, and to express hFlt3L and OX40L (MVAΔC7LΔE5R-hFlt3L-OX40L); a recombinant MVA genetically engineered to comprise a deletion of E5R (MVAΔE5R); a recombinant MVA genetically engineered to comprise a deletion of E5R and to express hFlt3L and OX40L (MVAΔE5R-hFlt3L-OX40L); a recombinant vaccinia virus comprising a deletion of C7L (VACVΔC7L) genetically engineered to express OX40L (VACVΔC7L-OX40L); a recombinant VACVΔC7L genetically engineered to express both OX40L and hFlt3L (VACVΔC7L-hFlt3L-OX40L); a VACV genetically engineered to comprise a deletion of E5R (VACVΔE5R); a recombinant VACV genetically engineered to comprise a deletion of E5R, a deletion of thymidine kinase (TK), and to express anTi-CTLA-4, hFlt3L, and OX40L (VACV-TK⁻-anti-CTLA-4-ΔE5R-hFlt3L-OX40L); a MYXV genetically engineered to comprise a deletion of M31R (MYXVΔM31R); a recombinant MYXV genetically engineered to comprise a deletion of M31R and to express hFl3L and OX40L (MYXVΔM31R-hFlt3L-OX40L); and/or additional engineered poxviruses selected from MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, or combinations thereof. In some embodiments, the treatment comprises one or more of the following: inducing an immune response in the subject against the tumor or enhancing or promoting an ongoing immune response against the tumor in the subject, reducing the size of the tumor, eradicating the tumor, inhibiting growth of the tumor, inhibiting metastatic growth of the tumor, inducing apoptosis of tumor cells, or prolonging survival of the subject. In some embodiments, the induction, enhancement, or promotion of the immune response comprises one or more of the following: increased levels of effector T-cells in tumor cells as compared to tumor cells infected with the corresponding MVA, MVAΔE3L, MVAΔC7L, VACV, VACVΔC7L, or MYXV strain; and increased splenic production of effector T-cells as compared to the corresponding MVA, MVAΔE3L, MVAΔC7L, VACV, VACVΔC7L, or MYXV strain. In some embodiments, the subject is a human. In some embodiments, the composition of the present technology comprising MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 is administered to the subject by intratumoral or intravenous injection or a simultaneous (i.e., concurrent) or sequential combination of intratumoral and intravenous injection.

In some embodiments, the subject is diagnosed with a cancer such as melanoma, colon carcinoma, breast cancer, prostate cancer, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor and other bone tumors (e.g., osteosarcoma, malignant fibrous histiocytoma), leiomyosarcoma, rhabdomyosarcoma, pancreatic cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, brain/CNS tumors (e.g., astrocytoma, glioma, glioblastoma, childhood tumors, such as atypical teratoid/rhabdoid tumor, germ cell tumor, embryonal tumor, ependymoma) medulloblastoma, craniopharyngioma, ependymoma, pinealoma, hemangioblastoma, acoustic neuroma, oligodendroglioma, meningioma, neuroblastoma, retinoblastoma, head-and-neck cancer, rectal adenocarcinoma, glioma, urothelial carcinoma, uterine (e.g., endometrial cancer, fallopian tube cancer) non-small cell lung cancer (squamous and adenocarcinoma), ductal carcinoma in situ, and hepatocellular carcinoma, adrenal tumors (e.g., adrenocortical carcinoma), esophageal cancer, eye cancer (e.g., melanoma, retinoblastoma), gallbladder cancer, gastrointestinal cancer, heart cancer, laryngeal and hypopharyngeal cancer, oral cancer (e.g., lip, mouth, salivary gland), nasopharyngeal cancer, neuroblastoma, peritoneal cancer, pituitary cancer, Kaposi's sarcoma, small intestine cancer, stomach cancer, thymus cancer, thyroid cancer, parathyroid cancer, vaginal tumor, and the metastases of any of the foregoing.

### XVI. Combination Therapy with Other Active Agents

In some embodiments, the engineered poxviruses of the present technology (e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4) are combined or separately, sequentially, or simultaneously (i.e., concurrently) administered with any combination of: (i) one or more immune checkpoint blocking agents and/or one or more immune system stimulators; (ii) one or more anti-cancer drugs; and (iii) an immunomodulatory drug (i.e., fingolimod (FTY720)). In some embodiments, the combined administration of the engineered poxviruses of the present technology (e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4) with any one or more of: (i) one or more immune checkpoint blocking agents and/or one or more immune system stimulators; (ii) one or more anti-cancer drugs; and (iii) an immunomodulatory drug (i.e., fingolimod (FTY720)) results in a synergistic effect with respect to the treatment of solid tumors.

### A. Immune Checkpoint Blocking Agents and Immune System Stimulators

In some embodiments, MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 is combined or separately, sequentially, or simultaneously (i.e., concurrently) administered with one or more immune checkpoint blocking agents and/or one or more immune system stimulators. The one or more immune checkpoint blocking agents may target any one or more of PD-1 (programmed death 1), PD-L1 (programmed death ligand 1), or CTLA-4 (cytotoxic T lymphocyte antigen 4) (e.g., anti-huPD-1, anti-huPD-L1, or anti-huCTLA-4 antibodies).

In some embodiments, the one or more immune checkpoint blocking agents are selected from the group consisting of ipilimumab, nivolumab, pidilizumab, lambrolizumab, pembrolizumab, atezolizumab, avelumab, and durvalumab, MPDL3280A, BMS-936559, MEDI-4736, MSB 00107180, inhibitory antibodies against LAG-3 (lymphocyte activation gene 3), TIM3 (T-cell immunoglobulin and mucin-3), B7-H3, B7-H4, TIGIT (T-cell immunoreceptor with Ig and ITIM domains), AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS (inducible T-cell costimulatory), DLBCL (diffuse large B-cell lymphoma) inhibitors, BTLA (B and T lymphocyte attenuator), PDR001, and any combination thereof. Dosage ranges of the foregoing are known in or readily within the skill in the art as several dosing clinical trials have been completed, making extrapolation to other agents possible.

By way of example, but not by way of limitation, in some embodiments, the one or more immune system stimulators are selected from among a natural killer cell (NK) stimulator, an antigen presenting cell (APC) stimulator, a granulocyte macrophage colony-stimulating factor (GM-CSF), and a toll-like receptor stimulator.

In some embodiments, the NK stimulator includes, but is not limited to, IL-2, IL-15, IL-15/IL-15RA complex, IL-18, and IL-12. In some embodiments, the NK stimulator includes an antibody that stimulates at least one of the following receptors NKG2, KIR2DL1/S1, KRI2DL5A, NKG2D, NKp46, NKp44, or NKp30.

In some embodiments, the APC stimulator includes, but is not limited to, CD28, ICOS, CD40, CD30, CD27, OX-40, and 4-1BB.

In some embodiments, the combination of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 and one or more immune checkpoint inhibitors and/or one or more immune system stimulators results in a synergistic effect. In some embodiments, the combination of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 and one or more immune checkpoint inhibitors and/or one or more immune system stimulators results in an enhanced anti-tumor effect. In some embodiments, the combination of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 and anti-PD-L1 results in a synergistic effect with respect to the treatment of solid tumors. In some embodiments, the combination of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 and anti-PD-1 results in a synergistic effect with respect to the treatment of solid tumors. In some embodiments, the combination of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 and anti-CTLA-4 results in a synergistic effect with respect to the treatment of solid tumors.

In some embodiments, the combination of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 with one or more immune checkpoint blocking agents and/or one or more immune system stimulators is further combined or separately, sequentially, or simultaneously (i.e., concurrently) administered with one or more anti-cancer drugs and/or immunomodulatory drugs described below in Sections XVI B and C. In some embodiments, the combination of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 with one or more immune checkpoint blocking agents and/or one or more immune system stimulators and one or more anti-cancer drugs and/or immunomodulatory drugs described below in Sections XVI B and C results in a synergistic effect with respect to the treatment of solid tumors.

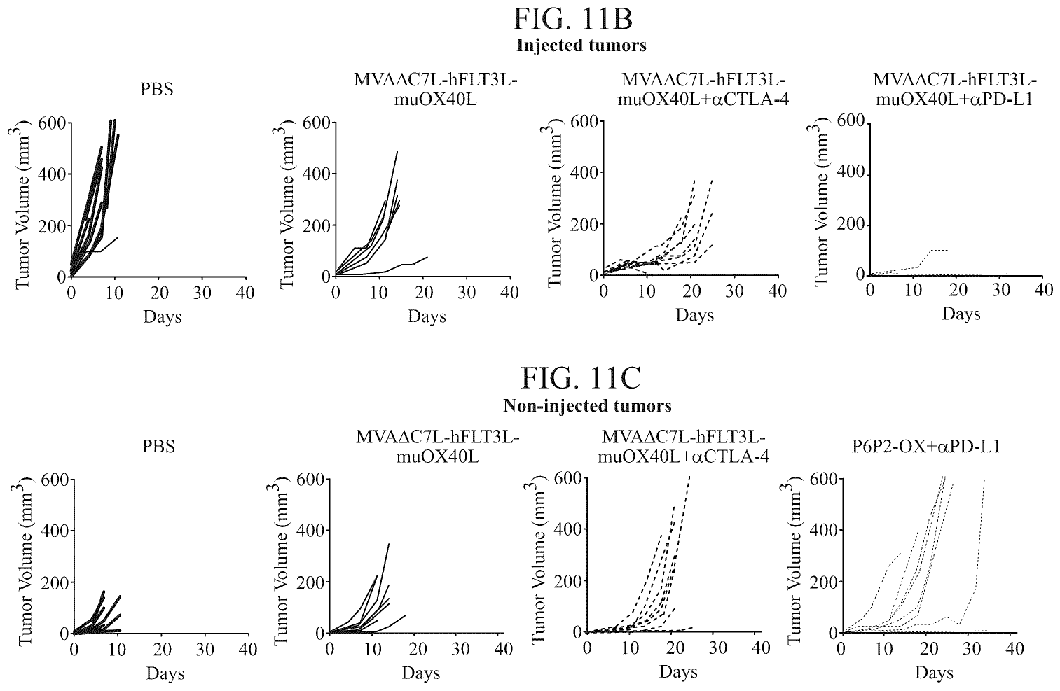

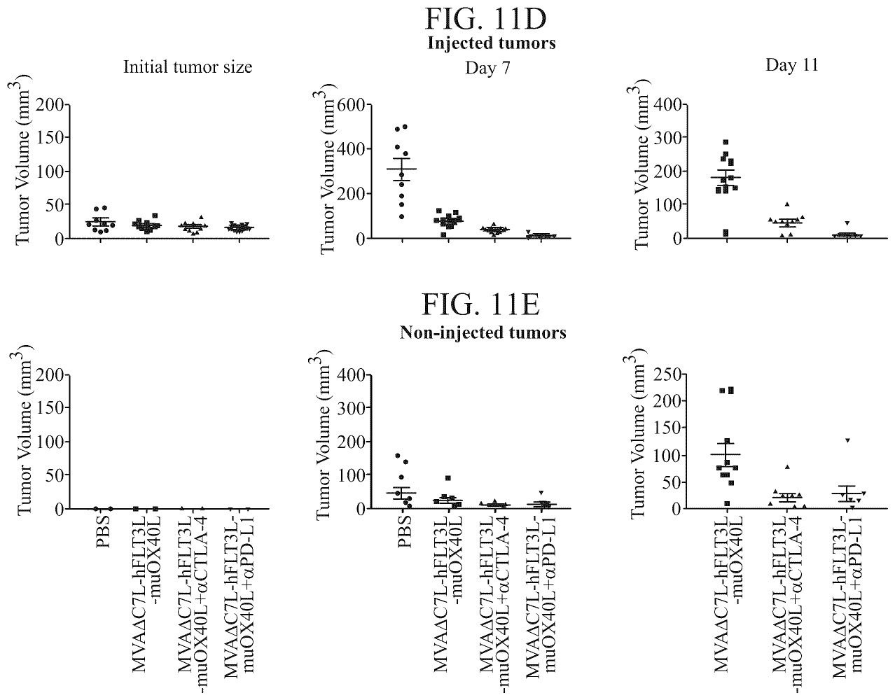

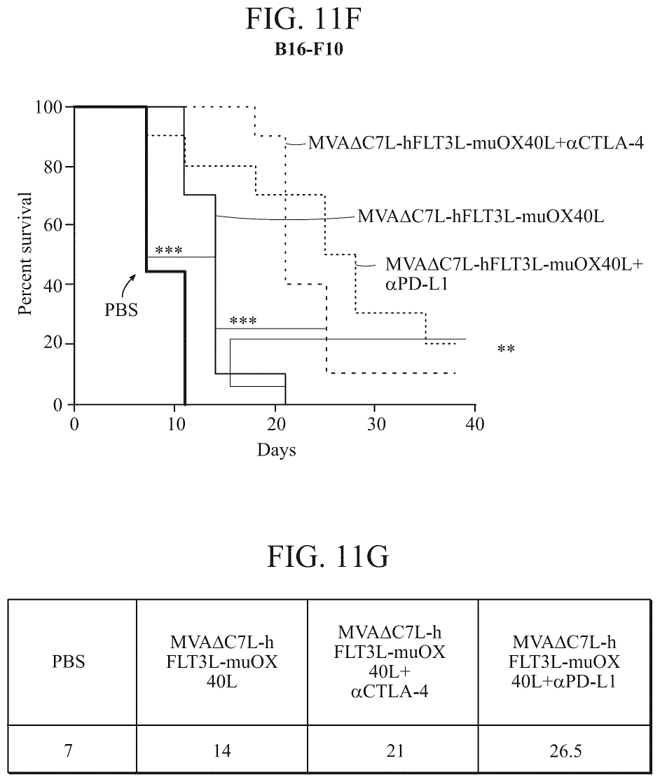

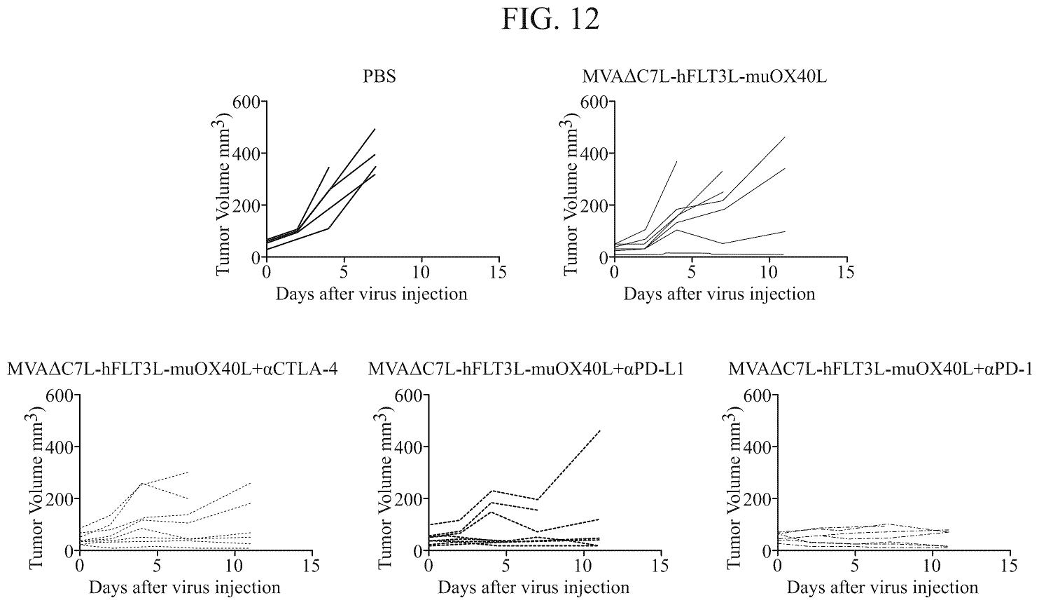

It has been reported that the sequential (i.e., serial) administration of anti-OX40 antibody followed by the immune checkpoint inhibitor, anti-PD-1 antibody, improves the therapeutic efficacy of the combination, resulting in delayed tumor progression and, in some cases, complete tumor regression. (See, e.g., Shrimali et al., Cancer Immunol. Res.5(9): OF1-OF12 (2017); Messenheimer et al., Clin. Cancer Res. 23(20):6165-6177 (2017)). However, the same studies show that the simultaneous (i.e., concurrent) administration of anti-OX40 antibody and anti-PD-1 antibody negates the anti-tumor effects of OX40 antibody and results in poor treatment outcomes in mice. (See, Shrimali et al., (2017); Messenheimer et al., (2017)). By contrast, as shown in FIGS. 11B-11G, the combined, simultaneous (i.e., concurrent) administration of the viruses expressing the OX40L transgene of the present technology (e.g., MVAΔC7L-hFlt3L-OX40L) and an immune checkpoint inhibitor (e.g., MVAΔC7L-hFlt3L-OX40L+anti-PD-L1 or MVAΔC7L-hFlt3L-OX40L+anti-CTLA-4 or MVAΔC7L-hFlt3L-OX40L+anti-PD-1 (not shown)) in a mouse melanoma model surprisingly and unexpectedly results in enhanced anti-tumor effects in both injected and non-injected tumors and increased survival as compared to controls. In some embodiments, the combination of the viruses expressing the OX40L transgene of the present technology, e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACV-TK⁻-anti-CTLA-4-ΔE5R-hFlt3L-OX40L, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R-hFlt3L-OX40L, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and MYXVΔM62RΔM63RΔM64RΔM31R-hFlt3L-OX40L-IL-12-IL-15/IL-15Rα-anti-CTLA-4 and/or and one or more immune checkpoint inhibitors (e.g., anti-PD-L1 antibody, anti-PD-1 antibody, anti-CTLA-4 antibody) results in a surprising and unexpected enhanced anti-tumor effect as compared to the combination of an immune checkpoint inhibitor and anti-OX40 agonist antibody. In some embodiments, the combination of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACV-TK⁻-anti-CTLA-4-ΔE5R-hFlt3L-OX40L, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R-hFlt3L-OX40L, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and MYXVΔM62RΔM63RΔM64RΔM31R-hFlt3L-OX40L-IL-12-IL-15/IL-15Rα-anti-CTLA-4 and one or more immune checkpoint inhibitors (e.g., anti-PD-L1 antibody, anti-PD-1 antibody, anti-CTLA-4 antibody) results in a surprising and unexpected synergistic effect with respect to the treatment of solid tumors as compared to the combination of an immune checkpoint inhibitor and anti-OX40 agonist antibody.

### B. Anti-Cancer Drugs

Receptor tyrosine kinases, such as EGFR and HER2, have been implicated in promoting tumor cell proliferation and survival through the Ras-Raf-Mek-Erk (Ras-MAPK) pathway. Raf and Mek are also targets for inhibiting oncogenic signals arising from upstream receptor tyrosine kinases or from gain-of-function mutations in RAS or RAF that drive Ras-MAPK signaling. The identification of key activating mutations in cancers including melanoma have led to the development of targeted therapies along the MAPK-pathway. For example, activating mutations in BRAF occur in over half of the melanoma cancers, a majority of which include the BRAF^{V600E} mutation, which constitutively activates the MAPK signaling pathway. This in turn leads to increased metastatic behavior including invasiveness, while reducing apoptosis (i.e., increasing cancer cell survival). Several anti-cancer drugs have been developed to mitigate the pathogenic signaling from this pathway. Focused therapies targeting this pathway include inhibitors of EGFR, HER2, BRAF, RAF, and MEK.

Immunotherapies consisting of checkpoint inhibitors (PD-1/PD-L1, CTLA-4) and combinations of MAPK-pathway targeted therapies have shown promising results in, for example, BRAF-positive advanced melanoma. It has been reported that MAPK pathway activation contributes to immune escape, while MAPK pathway inhibition contributes to a more favorable immune environment via abrogation of immunosuppressive factors as well as dysregulation of certain other immunoregulatory proteins such as PD-L1. Furthermore, oncolytic herpes virus (T-Vec) in dual combination with MAPK pathway inhibition has been shown in preclinical models to increase cancer cell death as compared with single agent alone, while the triple combination of MAPK-inhibitor, T-Vec, and a checkpoint inhibitor (e.g., anti-PD-L1 antibody) showed synergistic immunostimulatory effects. Robust immune response with MAPK pathway inhibition has been strongly associated with increased activation of CD8 T-cell influx and increased levels of secreted IFN and TNF-α.

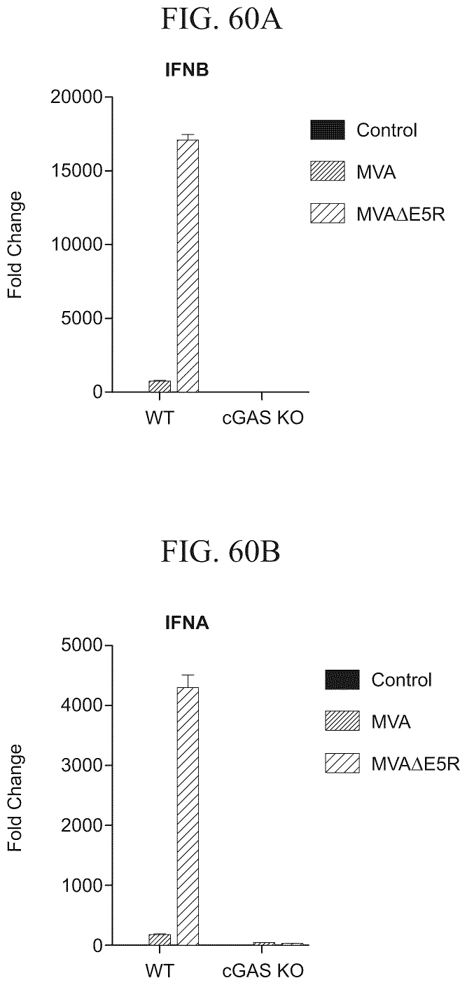

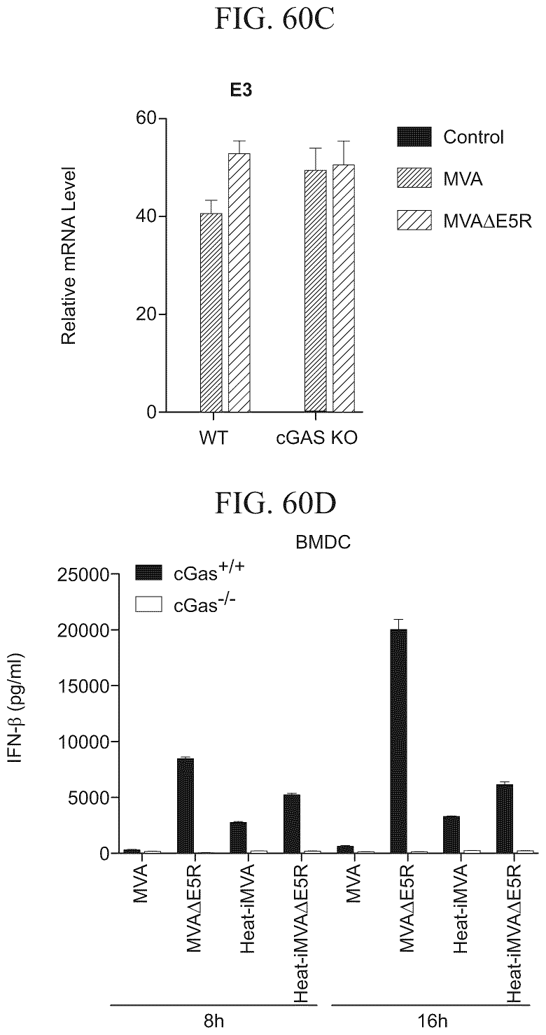

As demonstrated herein, the recombinant viral constructs of the present technology comprising deletions of E5R (or its orthologue), such as MVAΔE5R, VACVΔE5R, and MYXVΔM31R, significantly increase IFN gene expression levels greater than 1000-fold compared to a corresponding wild-type virus (see FIG. 60A).

In some embodiments, MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 is combined or separately, sequentially, or simultaneously (i.e., concurrently) administered with one or more anti-cancer drugs selected from the group consisting of Mek inhibitors (e.g., U0126, selumitinib (AZD6244), PD98059, trametinib, cobimetinib), EGFR inhibitors (e.g., lapatinib (LPN), erlotinib (ERL)), HER2 inhibitors (e.g., lapatinib (LPN), Trastuzumab), Raf inhibitors (e.g., sorafenib (SFN)), BRAF inhibitors (e.g., dabrafenib, vemurafenib), an anti-OX40 antibody, a GITR agonist antibody, an anti-CSFR antibody, a CSFR inhibitor, paclitaxel, TLR9 agonist CpG, and VEGF inhibitors (e.g., Bevacizumab).

In some embodiments, the combination of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 with one or more anti-cancer drugs is further combined or separately, sequentially, or simultaneously (i.e., concurrently) administered with one or more immune checkpoint blocking agents and/or one or more immune system stimulators as described in Section XVI A. In some embodiments, the combination of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 with the one or more anti-cancer drugs and one or more immune checkpoint blocking agents and/or one or more immune system stimulators results in a synergistic effect with respect to the treatment of solid tumors.

In some embodiments, the combination of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 with one or more anti-cancer drugs is further combined or separately, sequentially, or simultaneously (i.e., concurrently) administered with one or more immune checkpoint blocking agents and/or one or more immune system stimulators as described in Section XVI A, and/or immunomodulatory drugs described in Section XVI C. In some embodiments, the combination of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 with the one or more anti-cancer drugs, one or more immune checkpoint blocking agents or immune system stimulators and/or immunomodulatory drugs, results in a synergistic effect with respect to the treatment of solid tumors.

### C. Immunomodulatory Drugs

Fingolimod (FTY720) is an orally active immunomodulatory drug used to treat multiple sclerosis. Fingolimod acts as a sphingosine-1-phosphate receptor modulator which blocks lymphocyte egress from lymph nodes.

In some embodiments, MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 is combined or separately, sequentially, or simultaneously (i.e., concurrently) administered with FTY720.

In some embodiments, the combination of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 with FTY720 is further combined or separately, sequentially, or simultaneously (i.e., concurrently) administered with one or more immune checkpoint blocking agents and/or one or more immune system stimulators as described in Section XVI A and/or anti-cancer drugs described in Section XVI B. In some embodiments, the combination of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 with FTY720, one or more immune checkpoint blocking agents, and/or immune system stimulators, and/or anti-cancer drugs, results in a synergistic effect with respect to the treatment of solid tumors.

### XVII Kits Comprising Engineered MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 Viruses

The present disclosure provides for kits comprising one or more compositions comprising one or more of the engineered poxviruses, e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, described herein together with instructions for the administration of the engineered poxviruses to a subject to be treated. The instructions may indicate a dosage regimen for administering the composition or compositions as provided below.

In some embodiments, the kit may also comprise an additional composition comprising one or more immune checkpoint blocking agents and/or one or more immune system stimulators for conjoint administration with the engineered poxvirus, e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, composition.

In some embodiments, the kit may also comprise an additional composition comprising one or more anti-cancer drugs for conjoint administration with the engineered poxvirus, e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, composition.

In some embodiments, the kit may also comprise an additional composition comprising an immunomodulatory drug for conjoint administration with the engineered poxvirus, e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, composition.

In some embodiments, the kit may also comprise an additional composition comprising one or more immune checkpoint blocking agents and/or one or more immune system stimulators, and one or more anti-cancer drugs for conjoint administration with the engineered poxvirus, e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, composition.

In some embodiments, the kit may also comprise an additional composition comprising any combination of one or more immune checkpoint blocking agents and/or one or more immune system stimulators, one or more anti-cancer drugs, and an immunomodulatory drug for conjoint administration with the engineered poxvirus, e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, composition.

### XVIII. Effective Amount and Dosage of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4

In general, the subject is administered a dosage of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 in the range of about 10⁶ to about 10¹⁰ plaque forming units (pfu), although a lower or higher dose may be administered. In some embodiments, the dosage ranges from about 10² to about 10¹⁰ pfu. In some embodiments, the dosage ranges from about 10³ to about 10¹⁰ pfu. In some embodiments, the dosage ranges from about 10⁴ to about 10¹⁰ pfu. In some embodiments, the dosage ranges from about 10⁵ to about 10¹⁰ pfu. In some embodiments, the dosage ranges from about 10⁶ to about 10¹⁰ pfu. In some embodiments, the dosage ranges from about 10⁷ to about 10¹⁰ pfu. In some embodiments, the dosage ranges from about 10⁸ to about 10¹⁰ pfu. In some embodiments, the dosage ranges from about 10⁹ to about 10¹⁰ pfu. In some embodiments, dosage is about 10⁷ to about 10⁹ pfu. The equivalence of pfu to virus particles can differ according to the specific pfu titration method used. Generally, a pfu is equal to about 5 to 100 virus particles and 0.69 pfu is about 1 TCID50. A therapeutically effective amount of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 can be administered in one or more divided doses for a prescribed period of time and at a prescribed frequency of administration.

For example, as is apparent to those skilled in the art, a therapeutically effective amount of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 in accordance with the present disclosure may vary according to factors such as the disease state, age, sex, weight, and general condition of the subject, and the ability of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 to elicit a desired immunological response in the particular subject (the subject's response to therapy). In delivering MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 to a subject, the dosage will also vary depending upon such factors as the general medical condition, previous medical history, disease type and progression, tumor burden, the presence or absence of tumor infiltrating immune cells in the tumor, and the like.

In some embodiments, it may be advantageous to formulate compositions of the present disclosure in dosage unit form for ease of administration and uniformity of dosage. “Dosage unit form as used herein” refers to physically discrete units suited as unitary dosages for the mammalian subjects to be treated; each unit containing a predetermined quantity of active material calculated to produce the desired therapeutic effect in association with the required pharmaceutically or veterinary acceptable carrier.

### XIX. Administration and Therapeutic Regimen of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4

Pharmaceutical compositions are typically formulated to be compatible with its intended route of administration. Administration of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 can be achieved using more than one route. Examples of routes of administration include, but are not limited to parenteral (e.g., intravenous, intramuscular, intraperitoneal, intradermal, subcutaneous), intratumoral, intrathecal, intranasal, systemic, transdermal, iontophoretic, intradermal, intraocular, or topical administration. In one embodiment, MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 is administered directly into the tumor, e.g., by intratumoral injection, where a direct local reaction is desired. Additionally, administration routes of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 can vary, e.g., first administration using an intratumoral injection, and subsequent administration via an intravenous injection, or any combination thereof. A therapeutically effective amount of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 injection can be administered for a prescribed period of time and at a prescribed frequency of administration. In certain embodiments, MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 can be used in conjunction with other therapeutic treatments. For example, the recombinant poxvirus of the present technology can be administered in a neoadjuvant (preoperative) or adjuvant (postoperative) setting for subjects inflicted with bulky primary tumors. It is anticipated that such optimized therapeutic regimen will induce an immune response against the tumor and reduce the tumor burden in a subject before or after primary therapy, such as surgery. Furthermore, MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 can be administered in conjunction with other therapeutic treatments such as chemotherapy or radiation.

In certain embodiments, the MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 virus is administered at least once weekly or monthly but can be administered more often if needed, such as two times weekly for several weeks, months, years, or even indefinitely as long as benefits persist. More frequent administrations are contemplated if tolerated and if they result in sustained or increased benefits. Benefits of the present methods include but are not limited to the following: reduction of the number of cancer cells, reduction of the tumor size, eradication of tumor, inhibition of cancer cell infiltration into peripheral organs, inhibition or stabilization or eradication of metastatic growth, inhibition or stabilization of tumor growth, and stabilization or improvement of quality of life. Furthermore, the benefits may include induction of an immune response against the tumor, activation of effector CD4⁺ T-cells, an increase of effector CD8⁺ T-cells, or reduction of regulatory CD4⁺ cells. For example, in the context of melanoma, a benefit may be a lack of recurrences or metastasis within one, two, three, four, five, or more years of the initial diagnosis of melanoma. Similar assessments can be made for colon cancer and other solid tumors.

In certain other embodiments, the tumor mass or tumor cells are treated with MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 in vivo, ex vivo, or in vitro.

### XX. Vectors

In some embodiments, a pCB plasmid-based vector is used to insert a specific gene of interest (SG), such as OX40L (murine or human), under the control of the vaccinia synthetic early and late promoter (PsE/L). The methodology for constructing the vector has been described (See M. Puhlmann, C. K. Brown, M. Gnant, J. Huang, S. K. Libutti, H. R. Alexander, D. L. Bartlett, Vaccinia as a vector for tumor-directed gene therapy: Biodistribution of a thymidine kinase-deleted mutant Cancer Gene Therapy 7(1):66-73 (2000)). A xanthine-guanine phosphoribosyl transferase (gpt) gene under the control of vaccinia P7.5 promoter is used as a selectable marker. An illustrative pCB-mOX40L-gpt vector nucleic acid sequence is set forth in SEQ ID NO: 3. In some embodiments, a pUC57 plasmid-based vector is used to insert a specific gene of interest (SG), such as OX40L (murine or human), under the control of the vaccinia synthetic early and late promoter (PsE/L). In some embodiments, a pMA plasmid-based vector is used to insert a specific gene of interest (SG), such as OX40L (murine or human), under the control of the vaccinia synthetic early and late promoter (PsE/L). An mCherry gene under the control of vaccinia P7.5 promoter is used as a selectable marker. An illustrative pUC57-hOX40L-mCherry vector nucleic acid sequence is set forth in SEQ ID NO: 5. Additional illustrative vectors nucleic acid sequences of the present technology are shown in Tables 2-5.

In some embodiments, these expression cassettes are flanked by a partial sequence of TK gene on each side (TK-L, TK-R). Homologous recombination that occurs at the TK locus of the plasmid DNA and MVAΔE3L, MVAΔC7L, MVAΔE5R, VACVΔC7L, VACVΔE5R, or MYXVΔM31R genomic DNA results in the insertion of OX40L and selectable marker expression cassettes into the MVAΔE3L, MVAΔC7L, MVAΔE5R, VACVΔC7L, VACVΔE5R, or MYXVΔM31R genomic DNA TK locus to generate MVAΔE3L-TK(−)-OX40L, MVAΔC7L-TK(−)-OX40L, MVAΔE5R-TK(−)-OX40L, VACVΔC7L-TK(−)-OX40L, VACVΔE5R-TK(−)-OX40L, or MYXVΔM31R-TK(−)-OX40L. Additionally or alternatively, suitable loci other than the TK locus within the virus could be used. Homologous recombination that occurs at a suitable viral gene locus of the plasmid DNA and MVA, MVAΔE3L, MVAΔC7L, MVAΔE5R, VACV, VACVΔC7L, VACVΔE5R, MYXV, or MYXVΔM31R genomic DNA results in the insertion of one or more specific gene of interest (e.g., OX40L, hFlt3L, anti-CTLA-4, etc.) and/or selectable marker expression cassettes into the MVA, MVAΔE3L, MVAΔC7L, MVAΔE5R, VACVΔC7L, VACVΔE5R, MYXV, or MYXVΔM31R genomic DNA viral gene locus to generate recombinant poxviruses such as those described herein.

In some embodiments, position 18,407 to 18,859 of the MVA genomic sequence (SEQ ID NO: 1) is replaced with a heterologous nucleic acid sequence comprising one or more open reading frames that encode for a selectable marker, such as gpt or mCherry, and a gene of interest (SG), such as OX40L. In some embodiments, position 75,560 to 76,093 of the MVA genomic sequence (SEQ ID NO: 1) is replaced with a heterologous nucleic acid sequence comprising one or more open reading frames that encode for a selectable marker, such as gpt or mCherry, and a gene of interest (SG), such as OX40L. In some embodiments, position 15,716 to 16,168 of the VACV genomic sequence (SEQ ID NO: 2) is replaced with a heterologous nucleic acid sequence comprising one or more open reading frames that encode for a selectable marker, such as gpt or mCherry, and a gene of interest (SG), such as OX40L. In some embodiments, position 75,798 to 75,868 of the MVA genomic sequence (SEQ ID NO: 1) is replaced with a heterologous nucleic acid sequence comprising one or more open reading frames that encode for a selectable marker, such as gpt or mCherry, and a gene of interest (SG), such as OX40L. In some embodiments, position 80,962 to 81,032 of the VACV genomic sequence (SEQ ID NO: 2) is replaced with a heterologous nucleic acid sequence comprising one or more open reading frames that encode for a selectable marker, such as gpt or mCherry, and a gene of interest (SG), such as OX40L. The recombinant viruses are enriched by selection and plaque-purified for 4-5 rounds until the appropriate recombinant viruses are obtained.

Similarly, in some embodiments, a pUC57 plasmid-based vector is used to insert a specific gene of interest (SG), such as hFlt3L, into the MVA or VACV viral genome backbone. GFP may be used as a selectable marker. An illustrative pUC57-hFlt3L-GFP vector nucleic acid sequence is set forth in SEQ ID NO: 4. In some embodiments, these expression cassettes are flanked by a partial sequence of C7 gene on each side. Additionally or alternatively, suitable loci other than the C7 locus within the virus could be used. Homologous recombination that occurs at the C7 locus of the plasmid DNA and MVAΔE3L, MVAΔE5R, MVA, VACV, VACVΔC7L, VACVΔE5R, MYXV, or MYXVΔM31R genomic DNA results in the insertion of hFlt3L and selectable marker expression cassettes into the MVAΔE3L, MVAΔE5R, MVA, VACV, VACVΔC7L, VACVΔE5R, MYXV, or MYXVΔM31R genomic DNA C7 locus.

In some embodiments, position 18,407 to 18,859 of the MVA genomic sequence (SEQ ID NO: 1) is replaced with a heterologous nucleic acid sequence comprising one or more open reading frames that encode for a selectable marker, such as GFP, and a gene of interest (SG), such as hFlt3L. In some embodiments, position 75,560 to 76,093 of the MVA genomic sequence (SEQ ID NO: 1) is replaced with a heterologous nucleic acid sequence comprising one or more open reading frames that encode for a selectable marker, such as gpt or mCherry, and a gene of interest (SG), such as hFlt3L. In some embodiments, position 15,716 to 16,168 of the VACV genomic sequence (SEQ ID NO: 2) is replaced with a heterologous nucleic acid sequence comprising one or more open reading frames that encode for a selectable marker, such as GFP, and a gene of interest (SG), such as hFlt3L. In some embodiments, position 80,962 to 81,032 of the VACV genomic sequence (SEQ ID NO: 2) is replaced with a heterologous nucleic acid sequence comprising one or more open reading frames that encode for a selectable marker, such as GFP, and a gene of interest (SG), such as hFlt3L. The recombinant viruses are enriched by selection and plaque-purified for 4-5 rounds until the appropriate recombinant viruses are obtained.

In some embodiments, position 38,432 to 39,385 of the MVA genomic sequence (SEQ ID NO: 1) is replaced with a heterologous nucleic acid sequence comprising one or more open reading frames that encode for a selectable marker, such as mCherry, and/or a gene of interest (SG), such as hFlt3L and/or OX40L. The recombinant viruses are enriched by selection and plaque-purified for 4-5 rounds until the appropriate recombinant viruses are obtained.

In some embodiments, position 49,236 to 50,261 of the VACV genomic sequence (SEQ ID NO: 2) is replaced with a heterologous nucleic acid sequence comprising one or more open reading frames that encode for a selectable marker, such as mCherry, and/or a gene of interest (SG), such as hFlt3L and/or OX40L. The recombinant viruses are enriched by selection and plaque-purified for 4-5 rounds until the appropriate recombinant viruses are obtained.

In some embodiments, both a pCB-OX40L-gpt vector or a pUC57-OX40L-mCherry vector or a pUC57-OX40L-mCherry vector and a pUC57-hFlt3L-GFP vector are used to insert OX40L into the TK locus and hFlt3L into the C7 locus to generate MVAΔC7L-hFlt3L-TK(−)-OX40L or VACVΔC7L-hFlt3L-TK(−)-OX40L.

It will be appreciated, that any other expression vector suitable for integration into the MVA, VACV, or MYXV genome could be used as well as alternative promoters, regulatory elements, selectable markers, cleavage sites, and/or nonessential insertion regions of MVA, VACV, or MYXV. In some embodiments, the selectable marker is a reporter protein, wherein the reporter protein is a bioluminescent protein, a fluorescent protein, or a chemiluminescent protein. In some embodiments, the reporter protein is green fluorescent protein (GFP). In some embodiments, the selectable marker is xanthine-guanine phophoribosyl transferase gene (gpt). In some embodiments, the selectable marker is an mCherry gene. MVA, VAVC, and MYXV encode many immune modulatory genes at the ends of the linear genome, including C11, K3, F1, F2, F4, F6, F8, F9, F11, F14.5, J2, A46, E3L, B18R (WR200), E5R, K7R, C12L, B8R, B14R, N1L, C11R, K1L, C16, M1L, N2L, and WR199 (or their orthologs). These genes (or their orthologs) can be deleted to potentially enhance immune activating properties of the virus, and allow insertion of transgenes.

### XXI. Delivery of the Engineered Poxvirus Strains of the Present Technology as an Adjuvant to a Subject to Treat Cancer

### A. Compositions

### Immune-Activating Cancer Vaccine Adjuvants

Recent discoveries of cancer neoantigens have generated a renewed interest in cancer vaccination and the combination of cancer vaccination with immune checkpoint blockade to enhance vaccination effects. Developing effective vaccine adjuvants that can maximize antitumor immune responses is critical for the success of cancer vaccines.

Cancer vaccines comprise cancer antigens and immune adjuvants. Cancer antigens generally include tumor differentiation antigens, cancer testis antigens, neoantigens, and viral antigens in the case of tumors associated with oncogenic virus infection. Cancer antigens can be provided in the form of irradiated tumor cells, dendritic cells (DCs) loaded with tumor cell lysates or peptides, DNA or RNA encoding antigen, as well as oncolytic virus with transgene(s) encoding cancer antigen(s). Dendritic cells (DCs) are professional antigen-presenting cells that are important for priming naive T-cells to generate antigen-specific T-cell responses. Immune adjuvants are agents that promote antigen uptake by DCs and/or DC maturation and activation. Several immune adjuvants, including toll-like receptor (TLR) agonists, poly (I:C) (TLR3 agonist), CpG (TLR9 agonist), Imiquimod (TLR7 agonist), as well as STING agonists, have been shown to improve vaccine efficacy in preclinical models and clinical settings.

### Engineered Poxvirus Strains of the Present Technology as Adjuvant Therapy

The disclosure of the present technology relates to the use of the engineered poxvirus strains described herein (e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, and/or Heat-iMVAΔE5R) as vaccine adjuvants. In some embodiments, the disclosure of the present technology relates to the use of MVAΔC7L-hFlt3L-TK(−)-OX40L as a vaccine adjuvant. In some embodiments, the disclosure of the present technology relates to the use of MVAΔC7L-hFlt3L-TK(−)-OX40L in combination with Heat-inactivated vaccinia (Heat-iMVA, Heat-iMVAΔE5R) as a vaccine adjuvant. Heat-iMVA has been shown to induce type I IFN in conventional DCs (cDCs) via the cGAS/STING-dependent pathway and also induces type I IFN in plasmacytoid DCs (pDCs) via the TLR7/MyD88-dependent mechanism.

Moreover, intratumoral injection of Heat-iMVA eradicates injected tumors and leads to the generation of systemic antitumor immunity either as monotherapy or in combination with immune checkpoint blockade (ICB).

### Target Antigens

The compositions and methods disclosed herein are not intended to be limited by the choice of antigen or neoantigen. While numerous examples of antigens and neoantigens are provided, the skilled artisan can easily utilize the adjuvant disclosed herein with an antigen or neoantigen of choice. Exemplary, non-limiting target antigens that may be used in therapeutic regimens of the present technology include tumor differentiation antigens, cancer testis antigens, neoantigens, viral antigens in the case of tumors associated with oncogenic virus infection, GPA33, HER2/neu, GD2, MAGE-1, MAGE-3, BAGE, GAGE-1, GAGE-2, MUM-1, CDK4, N-acetylglucosaminyltransferase, p15, gp75, beta-catenin, ErbB2, cancer antigen 125 (CA-125), carcinoembryonic antigen (CEA), RAGE, MART (melanoma antigen), MUC-1, MUC-2, MUC-3, MUC-4, MUC-5ac, MUC-16, MUC-17, tyrosinase, tyrosinase-related proteins 1 and 2, Pmel 17 (gp100), GnT-V intron V sequence (N-acetylglucoaminyltransferase V intron V sequence), Prostate cancer psm, PRAME (melanoma antigen), β-catenin, EBNA (Epstein-Barr Virus nuclear antigen) 1-6, p53, kras, lung resistance protein (LRP) Bcl-2, prostate specific antigen (PSA), Ki-67, CEACAM6, colon-specific antigen-p (CSAp), NY-ESO-1, human papilloma virus E6 and E7, and combinations thereof. In some embodiments, the antigen comprises a neoantigen selected from the group consisting of M27 (REGVELCPGNKYEMRRHGTTHSLVIHD) (SEQ ID NO: 17), M30 (PSKPSFQEFVDWENVSPELNSTDQPFL) (SEQ ID NO: 18), M48 (SHCHWNDLAVIPAGVVHNWDFEPRKVS) (SEQ ID NO: 19), and combinations thereof. The target antigen may also be a fragment or fusion polypeptide comprising an immunologically active portion of the antigens listed above.

### Immune Checkpoint Blockade (ICB)

In some embodiments, the immunogenic compositions of the present technology further comprise one or more immune checkpoint blockade agents. Immune checkpoint blockade (ICB) antibodies have been at the forefront of immunotherapy and have been accepted as one of the pillars of cancer management options, including surgery, radiation, and chemotherapy. Because immune checkpoints have been implicated in the downregulation of antitumor immunity, agents and antibodies targeting immune checkpoint proteins or their ligands (CTLA-4, PD-1, or PD-L1) have been successful in disinhibiting antitumor T-cells, thereby leading to proliferation and survival of activated T-cells. This has led to the FDA approval of multiple immune checkpoint blockade (ICB) agents for patients with advanced cancers of various histological types, including melanoma, non-small cell lung cancer, renal cell carcinoma, Hodgkin lymphoma, head-and-neck cancer, urothelial carcinoma, Merkel cell carcinoma, PD-L1⁺ gastric adenocarcinoma, as well as mismatch repair deficient and microsatellite instability (MSI) high metastatic solid tumors.

Non-limiting examples of immune checkpoint blocking agents include agents or antibodies that modulate the activity of one or more checkpoint proteins including anti-PD-1 antibody, anti-PD-L1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001 and any combination thereof.

### Pharmaceutical Compositions and Preparations of the Present Technology

Disclosed herein are pharmaceutical compositions comprising an antigen and MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, and/or Heat-iMVAΔE5R as an adjuvant that may contain a carrier or diluent, which can be a solvent or dispersion medium containing, for example, water, saline, Tris buffer, polyol (for example, glycerol, propylene glycol, and liquid polyethylene glycol, and the like), suitable mixtures thereof, and vegetable oils. In some embodiments, the pharmaceutical compositions comprise an antigen and MVAΔC7L-hFlt3L-TK(−)-OX40L and Heat-iMVA as adjuvants. The proper fluidity can be maintained, for example, by the use of a coating such as lecithin, by the maintenance of the required particle size in the case of dispersion and by the use of surfactants. Prevention of the action of microorganisms can be effected by various antibacterial and antifungal agents and preservatives, for example, parabens, chlorobutanol, phenol, sorbic acid, thimerosal, and the like. In some embodiments, isotonic agents, for example, sugars or sodium chloride, and buffering agents are included. Prolonged absorption of the injectable compositions can be brought about by the use in the compositions of agents delaying absorption, for example, aluminum monostearate and gelatin or carrier molecules. Other excipients may include wetting or emulsifying agents. In general, excipients suitable for injectable preparations can be included as apparent to those skilled in the art.

Pharmaceutical compositions and preparations comprising an antigen and MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, and/or Heat-iMVAΔE5R as an adjuvant may be manufactured by means of conventional mixing, dissolving, granulating, emulsifying, encapsulating, entrapping or lyophilizing processes. Pharmaceutical compositions may be formulated in conventional manner using one or more physiologically acceptable carriers, diluents, excipients or auxiliaries that facilitate formulating preparations suitable for in vitro, in vivo, or ex vivo use. The compositions can be combined with one or more additional biologically active agents (for example parallel administration of GM-CSF) and may be formulated with a pharmaceutically acceptable carrier, diluent or excipient to generate pharmaceutical (including biologic) or veterinary compositions of the instant disclosure suitable for parenteral or intra-tumoral administration.

Many types of formulation are possible as is appreciated by those skilled in the art. The particular type chosen is dependent upon the route of administration chosen, as is well-recognized in the art. For example, systemic formulations will generally be designed for administration by injection, e.g., intravenous, as well as those designed for intratumoral delivery. In some embodiments, the systemic or intratumoral formulation is sterile.

Sterile injectable solutions are prepared by incorporating an antigen and MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, and/or Heat-iMVAΔE5R as an adjuvant in the required amount of the appropriate solvent with various other ingredients enumerated herein, as required, followed by suitable sterilization means. Generally, dispersions are prepared by incorporating the various sterilized active ingredients into a sterile vehicle that contains the basic dispersion medium and the required other ingredients from those enumerated above. In the case of sterile powders for the preparation of sterile injectable solutions, the preferred methods of preparation are vacuum drying and freeze-drying techniques, which yield a powder of the virus plus any additional desired ingredient from a previously sterile-filtered solution thereof.

In some embodiments, an antigen and MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 and/or Heat-iMVAΔE5R compositions of the present disclosure may be formulated in aqueous solutions, or in physiologically compatible solutions or buffers such as Hanks's solution, Ringer's solution, mannitol solutions or physiological saline buffer. In certain embodiments, any of the antigen and Heat-iMVA or Heat-iMVAΔE5R compositions may contain formulator agents, such as suspending, stabilizing, penetrating or dispersing agents, buffers, lyoprotectants or preservatives such as polyethylene glycol, polysorbate 80, 1-dodecylhexahydro-2H-azepin-2-one (laurocapran), oleic acid, sodium citrate, Tris HCl, dextrose, propylene glycol, mannitol, polysorbate polyethylenesorbitan monolaurate (Tween®-20), isopropyl myristate, benzyl alcohol, isopropyl alcohol, ethanol sucrose, trehalose and other such generally known in the art may be used in any of the compositions of the instant disclosure.

In some embodiments, the compositions of the present technology can be stored at −80° C. For the preparation of vaccine shots, e.g., 10²-10⁸ or 10²-10⁹ viral particles can be lyophilized, for example, in 100 ml of phosphate-buffered saline (PBS) in the presence of 2% peptone and 1% human albumin in an ampoule, preferably a glass ampoule. Alternatively, the injectable preparations can be produced by stepwise freeze-drying of the recombinant virus in a formulation. This formulation can contain additional additives such as mannitol, dextran, sugar, glycine, lactose or polyvinylpyrrolidone or other additives such as antioxidants or inert gas, stabilizers or recombinant proteins (e.g., human serum albumin) suitable for in vivo administration. The glass ampoule is then sealed and can be stored between 4° C. and room temperature for several months. In some embodiments, the ampoule is stored at temperatures below −20° C.

For therapy, the lyophilisate can be dissolved in an aqueous solution, such as physiological saline or Tris buffer, and administered either systemically or intratumorally. The mode of administration, the dose, and the number of administrations can be optimized by those skilled in the art.

The pharmaceutical compositions comprising an antigen and MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, and/or Heat-iMVAΔE5R as an adjuvant according to the present disclosure may comprise an additional adjuvant including aluminum salts, such as aluminum hydroxide or aluminum phosphate, Quil A, bacterial cell wall peptidoglycans, virus-like particles, polysaccharides, toll-like receptors, nano-beads, etc.

### Vaccines

In some embodiments, compositions comprising MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, and/or Heat-iMVAΔE5R adjuvant and one or more antigens are formulated into vaccines. In some embodiments, the compositions comprise MVAΔC7L-hFlt3L-TK(−)-OX40L as an adjuvant. In some embodiments, the compositions comprise MVAΔC7L-hFlt3L-TK(−)-OX40L and Heat-iMVA as adjuvants. In some embodiments, the vaccines are tumor antigen-containing whole cell vaccines (e.g., an irradiated whole cell vaccine). In some embodiments, the vaccines are administered to a subject to elicit an immune response against the antigens formulated therewith.

Effective Amount and Dosage of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, and/or Heat-iMVAΔE5R as a Cancer Vaccine Immune Adjuvant

In general, the subject is administered a dosage of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 and/or Heat-iMVAΔE5R in the range of about 10⁶ to about 10¹⁰ plaque forming units (pfu), although a lower or higher dose may be administered. In some embodiments, the dosage ranges from about 10² to about 10¹⁰ pfu. In some embodiments, the dosage ranges from about 10³ to about 10¹⁰ pfu. In some embodiments, the dosage ranges from about 10⁴ to about 10¹⁰ pfu. In some embodiments, the dosage ranges from about 10⁵ to about 10¹⁰ pfu. In some embodiments, the dosage ranges from about 10⁶ to about 10¹⁰ pfu. In some embodiments, the dosage ranges from about 10⁷ to about 10¹⁰ pfu. In some embodiments, the dosage ranges from about 10⁸ to about 10¹⁰ pfu. In some embodiments, the dosage ranges from about 10⁹ to about 10¹⁰ pfu. In some embodiments, dosage is about 10⁷ to about 10⁹ pfu. The equivalence of pfu to virus particles can differ according to the specific pfu titration method used. Generally, a pfu is equal to about 5 to 100 virus particles and 0.69 PFU is about 1 TCID50. A therapeutically effective amount of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, and/or Heat-iMVAΔE5R can be administered in one or more divided doses for a prescribed period of time and at a prescribed frequency of administration.

For example, as is apparent to those skilled in the art, a therapeutically effective amount of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, and/or Heat-iMVAΔE5R as an adjuvant in accordance with the present disclosure may vary according to factors such as the disease state, age, sex, weight, and general condition of the subject, and the ability of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, and/or Heat-iMVAΔE5R to elicit a desired immunological response in the particular subject (the subject's response to therapy). In delivering MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, and/or Heat-iMVAΔE5R to a subject, the dosage will also vary depending upon such factors as the general medical condition, previous medical history, disease type and progression, tumor burden, the presence or absence of tumor infiltrating immune cells in the tumor, and the like.

In some embodiments, it may be advantageous to formulate compositions of the present disclosure in dosage unit form for ease of administration and uniformity of dosage. “Dosage unit form as used herein” refers to physically discrete units suited as unitary dosages for the mammalian subjects to be treated; each unit containing a predetermined quantity of active material calculated to produce the desired therapeutic effect in association with the required pharmaceutically or veterinary acceptable carrier.

Administration and Therapeutic Regimen of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, and/or Heat-iMVAΔE5R 40L as a Cancer Vaccine Immune Adjuvant

A pharmaceutical composition is typically formulated to be compatible with its intended route of administration. Administration of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, and/or Heat-iMVAΔE5R as an adjuvant in an immunogenic composition (e.g., vaccine) can be achieved using more than one route. Examples of routes of administration include, but are not limited to parenteral (e.g., intravenous, intramuscular, intraperitoneal, intradermal, subcutaneous), intratumoral, intrathecal, intranasal, systemic, transdermal, iontophoretic, intradermal, intraocular, or topical administration. In one embodiment, the pharmaceutical composition of the present technology comprising an antigen and MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, and/or Heat-iMVAΔE5R as an adjuvant is administered directly into the tumor, e.g. by intratumoral injection, where a direct local reaction is desired. In some embodiments, the pharmaceutical composition of the present technology comprising an antigen and MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, and/or Heat-iMVAΔE5R as an adjuvant is administered peripherally relative to tumor beds.

Additionally, the administration routes can vary, e.g., first administration using an intratumoral injection, and subsequent administration via an intravenous injection, or any combination thereof. A therapeutically effective amount of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, and/or Heat-iMVAΔE5R as an adjuvant in a cancer vaccine injection can be administered for a prescribed period of time and at a prescribed frequency of administration. In certain embodiments, the pharmaceutical compositions of the present technology can be used in conjunction with other therapeutic treatments such as chemotherapy or radiation. In some embodiments, the pharmaceutical compositions of the present technology comprising a therapeutically effective amount of MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, and/or Heat-iMVAΔE5R as an adjuvant can be used in conjunction with immune checkpoint blockade therapy.

In certain embodiments, the pharmaceutical composition comprising an antigen and MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, and/or Heat-iMVAΔE5R as an adjuvant is administered at least once weekly or monthly but can be administered more often if needed, such as two times weekly for several weeks, months, years or even indefinitely as long as benefits persist. More frequent administrations are contemplated if tolerated and if they result in sustained or increased benefits. Benefits of the present methods include but are not limited to the following: reduction of the number of cancer cells, reduction of the tumor size (e.g., tumor volume), eradication of tumor, inhibition of cancer cell infiltration into peripheral organs, inhibition or stabilization or eradication of metastatic growth, inhibition or stabilization of tumor growth, and stabilization or improvement of quality of life. Furthermore, the benefits may include induction of an immune response against the tumor, increased IFN-γ⁺CD8⁺ T-cells, increased IFN-γ⁺CD4⁺ T-cells, activation of effector CD4⁺ T-cells, an increase of effector CD8⁺ T-cells, or reduction of regulatory CD4⁺ cells. For example, in the context of melanoma, a benefit may be a lack of recurrences or metastasis within one, two, three, four, five or more years of the initial diagnosis of melanoma. Similar assessments can be made for colon cancer and other solid tumors.

### B. Methods

In one aspect, the present disclosure provides for a method for treating solid tumor by enhancing an immune response in a subject in need thereof, the method comprising administering to the subject an immunogenic composition comprising one or more antigens and an adjuvant comprising MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, and/or Heat-iMVAΔE5R, thereby treating the tumor by enhancing immune response. In some embodiments, the adjuvant comprises MVAΔC7L-hFlt3L-TK(−)-OX40L. In some embodiments, the adjuvant comprises MVAΔC7L-hFlt3L-TK(−)-OX40L and Heat-iMVA.

In some embodiments, the disclosure provides methods comprising administering the immunogenic composition comprising one or more antigens and MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, and/or Heat-iMVAΔE5R as an adjuvant to a subject in order to elicit an immune response against the antigens.

In some embodiments of the methods disclosed herein, the administration step comprises administering the immunogenic composition in multiple doses.

In some embodiments, the methods described herein further comprise administering to the subject an immune checkpoint blockade agent selected from the group consisting of anti-PD-1 antibody, anti-PD-L1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof. In some embodiments, the immunogenic composition is delivered to the subject separately, sequentially, or simultaneously with the administration of the immune checkpoint blockade agent.

### C. Kits

In some embodiments, kits are provided. In some embodiments, the kit includes a container means and a separate portion of each of: (a) an antigen and (b) an adjuvant comprising MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4, and/or Heat-iMVAΔE5R. In some embodiments, the adjuvant comprises MVAΔC7L-hFlt3L-TK(−)-OX40L. In some embodiments, the adjuvant comprises MVAΔC7L-hFlt3L-TK(−)-OX40L and Heat-iMVA.

### EXPERIMENTAL EXAMPLES

The present technology is further illustrated by the following examples, which should not be construed as limiting in any way.

### General Materials and Methods

Viruses and Cell lines. MVA virus was kindly provided by Gerd Sutter (University of Munich), and propagated in BHK-21 (baby hamster kidney cell, ATCC CCL-10) cells. MVA is commercially and/or publicly available. MVAΔE3L virus was kindly provided by Gerd Sutter (University of Munich), and propagated in BHK-21 cells. The method of generation of MVAΔE3L Viruses was described (Hornemann et al., 2003). The viruses were purified through a 36% sucrose cushion. Heat-iMVA is generated by incubating purified MVA virus at 55° C. for 1 hour. BHK-21 were cultured in Eagle's Minimal Essential Medium (Eagle's MEM, can be purchased from Life Technologies, Cat #11095-080) containing 10% FBS, 0.1 mM nonessential amino acids (NEAA), and 50 mg/ml gentamycin. The murine melanoma cell line B16-F10 was originally obtained from I. Fidler (MD Anderson Cancer Center). B16-F10 cells were maintained in RPMI 1640 medium supplemented with 10% FBS, 100 Units/ml penicillin, 100 μg/ml streptomycin, 0.1 mM NEAA, 2 mM L-glutamine, 1 mM sodium pyruvate, and 10 mM HEPES buffer. All cells were grown at 37° C. in a 5% CO₂ incubator. The human melanoma SK-MEL-28 cells were cultured in complete RPMI 1640 medium. For human monocyte-derived dendritic cell culture, complete RPMI 1640 was supplemented with 10 mM Hepes, 1% penicillin/streptomycin (Media Lab, MSKCC, New York, NY), 50 μM 2-ME (GibcoBRL Life Technologies, Carlsbad, CA), 1% L-glutamine (GibcoBRL), and heat-inactivated, normal human serum (1% or 10%, v/v as specified for a particular experiment; Gemini Bio-Products, West Sacramento, CA). Sterile, recombinant, endotoxin-, pyrogen-, mycoplasma-, and carrier-free human cytokines were used to generate immature and mature blood moDCs. All media and reagents were endotoxin-free. All cells were grown at 37° C. in a 5% C02 incubator.

Mice. Female C57BL/6J mice between 6 and 10 weeks of age were purchased from the Jackson Laboratory and were used for in vivo experiments. These mice were maintained in the animal facility at the Sloan Kettering Institute. All procedures were performed in strict accordance with the recommendations in the Guide for the Care and Use of Laboratory Animals of the National Institute of Health. The protocol was approved by the Committee on the Ethics of Animal Experiments of Sloan-Kettering Cancer Institute.

Generation of recombinant MVAΔE3L-TK(−)-mOX40L virus. BHK21 cells were passaged into a 6-well plate. The next day, cells were infected with MVAΔE3L at multiplicity of infection (MOI) of 0.5. After 1-2 h, cells were transfected with 0.75 g pCB-mOX40L-gpt construct with 2 μl lipofectamine 2000. After 2 days, cells were collected and freeze/thaw three times. To select pure MVAΔE3L-mOX40L, recombinant viruses were selected through further culturing in gpt selection medium including MPA, xanthine and hypoxanthine, and plaque purified after 4-6 rounds of selection. PCR analysis was performed to verify that MVAΔE3L-TK(−)-mOX40L lacks part of the TK gene and with mOX40L insertion.

PCR verification of recombinant virus MVAΔE3L-mOX40L. PCR reactions were used to verify the purity of MVAΔE3L-TK(−)mOX40L recombinant virus. The primer sequences used for the PCR reactions are: TK-F4: 5′-TTGTCATCATGAACGGCGGA-3′ (SEQ ID NO: 11), TK-R4: 5′-TCCTTCGTTTGCCATACGCT-3′ (SEQ ID NO: 12), OX40L-F: 5′-CGTTGTAAGCGGCATCAAGG-3′ (SEQ ID NO: 13), OX40L-R: 5′-AAGGCCAGTGAAGCGACTAC-3′ (SEQ ID NO: 14).

FACS analysis of expression of mOX40L and hFlt3L. Murine B16-F10 melanoma cells (1×10⁶) or SK-MEL-28 cells were infected with MVAΔE3L, MVAΔE3L-TK(−)-mOX40L, MVAΔC7L, MVAΔC7L-hFlt3L, or MVAΔC7L-hFlt3L-TK(−)-mOX40L at a MOI of 10. Cells were collected at 24 h post infection and stained with PE-conjugated anti-murine OX40L or anti-hFlt3L antibody prior to FACS analysis.

Tumor implantation and intratumoral injection with viruses for evaluation of tumor infiltrating lymphocytes by flow cytometry analysis. A bilateral tumor implantation model was used in this technology. B16-F10 melanoma cells were implanted intradermally into the shaved skin on the right (5×10⁵ cells) and left (2.5×10⁵ cells) flanks of a C57BL/6J mouse. After 7 days post implantation, the larger tumors on the right flank (about 3 mm or larger in diameter) were injected twice per week with recombinant viruses or PBS when the mice were under anesthesia. Two or three days after the second injection, tumors were harvested and weighed. They were then minced prior to incubation with Liberase (1.67 Wünsch U/ml) and DNase (0.2 mg/ml) in serum free RPMI medium for 30 minutes at 37° C. Cell suspensions were generated by mashing through a 70 μm nylon filter, and then washed with the complete RPMI medium. Cells were processed for surface labeling with anti-CD3, CD45, CD4, and CD8 antibodies, and also for intracellular Granzyme B staining. Live cells are distinguished from dead cells by using fixable dye eFluor506 (eBioscience, Thermo Fisher Scientific, Waltham, MA). They were further permeabilized using permeabilization kit (eBioscience, Thermo Fisher Scientific, Waltham, MA), and stained for Granzyme B. Data were acquired using the LSRII Flow cytometer (Becton-Dickinson Biosciences, Franklin Lakes, NJ). Data were analyzed with FlowJo software (FlowJo, Becton-Dickinson, Franklin Lakes, NJ).

IFN-γ ELISPOT assay. B16-F10 melanoma cells were implanted intradermally to the right (5×10⁵ cells) and left (2.5×10⁵ cells) flanks of C57B/6J mice. Seven days after tumor implantation, the tumors on the right flanks were injected with PBS, or recombinant viruses. The injections were repeated once 3 days later. Two or three days after the second injection, spleens were harvested from mice treated with different viruses, and were mashed through a 70 m strainer (Thermo Fisher Scientific, Waltham, MA). Red blood cells were lysed using ACK Lysis Buffer (Life Technologies, Carlsbad, CA) and the cells were re-suspended in complete RPMI medium. CD8⁺ T cells were purified using CD8a (Ly-2) MicroBeads from Miltenyi Biotechnology. Enzyme-linked ImmunoSpot (ELISPOT) assay was performed to measure tumor specific IFN-γ⁺ CD8⁺ T cell activities according to the manufacturer's protocol (Becton-Dickinson Biosciences, Franklin Lakes, NJ). CD8⁺ T cells were mixed with irradiated B16 cells at 1:1 ratio (250,000 cells each) in RPMI medium, and the ELISPOT plate was incubated at 37° C. for 16 hours before staining.

Generation of recombinant MVAΔC7L-hFt3L-TK(−)-mOX40L virus. Two-step recombination was used to generate this virus. The first step is to generate MVAΔC7L-hFlt3L. pUC57 vector was constructed to insert an expression cassette into the C7L locus of MVA, which includes hFlt3L gene under the vaccinia viral synthetic early and late promoter (PsE/L) and GFP under the control of the vaccinia P7.5 promoter used as a selection marker. This expression cassette was flanked by partial sequence of C8L and C6R on the left and right side of C7L gene. BHK21 cells were infected with MVA at a multiplicity of infection (MOI) of 0.5 for 1 h, and then were transfected with the plasmid DNAs described above. The infected cells were collected at 48 h. Recombinant viruses were selected by serial selection of GFP⁺ foci. PCR analysis was performed to verify that MVAΔC7L-hFlt3L lacks the C7L gene and with hFlt3L insertion. The second step to generate MVAΔC7L-hFlt3L-TK(−)-mOX40L. The pCB plasmid containing a codon optimized mOX40L gene under the control of the vaccinia PsE/L as well as the E. coli xanthine-guanine phosphoribosyl transferase gene (gpt) under the control of vaccinia P7.5 promoter flanked by the thymidine kinase (TK) gene on either side was constructed. BHK21 cells were infected with MVAΔC7L-hFlt3L at a multiplicity of infection (MOI) of 0.5 for 1 h, and then were transfected with the plasmid DNAs described above. The infected cells were collected at 48 h. Recombinant viruses were selected through further culturing in gpt selection medium including MPA, xanthine and hypoxanthine, and plaque purified. PCR analysis was performed to verify that MVAΔC7L-hFlt3L-TK(−)-mOX40L lacks C7L gene and part of the TK gene, but with both hFlt3L and mOX40L insertion. MVAΔC7L-hFlt3L-TK(−) was also constructed with pCB plasmid containing gpt gene under the control of vaccinia P7.5 promoter flanked by the TK gene on either side. PCR analysis was performed to verify that MVAΔC7L-hFlt3L-TK(−) lacks C7L gene and part of the TK gene, but with hFlt3L insertion.

Bilateral tumor implantation model and intratumoral injection with recombinant MVAΔC7L-hFlt3L-TK(−)-mOX40L in the presence or absence of immune checkpoint blockade. Briefly, B16-F10 melanoma cells were implanted intradermally to the left and right flanks of C57BL/6 mice (5×10⁵ to the right flank and 1×10⁵ to the left flank). 9 days after tumor implantation, the larger tumors on the right flank were intratumorally injected with 2×10⁷ pfu of MVAΔC7L-hFlt3L-TK(−)-mOX40L. Mice were also treated with intraperitoneal delivery of immune checkpoint blockade antibodies, including anti-CTLA-4 (100 μg per mouse per injection), or anti-PD-L1 (250 μg per mouse per injection) twice weekly. The tumor sizes were measured and the tumors were repeatedly injected twice a week. The survival of mice was monitored. Tumor volumes were calculated according the following formula: 1 (length)×w (width)×h(height)/2. Mice were euthanized for signs of distress or when the diameter of the tumor reached 10 mm.

Unilateral intradermal tumor implantation and intratumoral injection with viruses recombinant MVAΔC7L-hFlt3L-TK(−)-mOX40L in the presence or absence of immune checkpoint blockade for the treatment of large established tumors. B16-F10 melanoma (5×10⁵ cells) were implanted intradermally into the shaved skin on the right flank of WT C57BL/6J mice. After 9 days post implantation, when the tumors that are 5 mm in diameter, they will be injected with MVAΔC7L-hFlt3L-TK(−)-mOX40L (5×10⁷ pfu) or PBS when the mice were under anesthesia. Viruses were injected twice weekly. Mice were also treated with intraperitoneal delivery of immune checkpoint blockade antibodies, including anti-CTLA-4 (100 μg per mouse per injection), anti-PD-1 (250 μg per mouse per injection), or anti-PD-L1 (250 μg per mouse per injection) twice weekly. Tumor volumes were calculated according the following formula: 1 (length)×w (width)×h(height)/2. The survival of mice was monitored. Mice were euthanized for signs of distress or when the diameter of the tumor reached 10 mm.

Preparation of primary chicken embryo fibroblasts (CEFs). Day 9-11 days of chicken embryos from SPF eggs (Charles River, Cat #10100326) were used. Embryos were minced by squeezing through a 10-cc syringe into a sterile 50 mL-EP tube. After digestion with 2.5% trypsin/EDTA at 37° C. for 5 min, cell suspensions were filtered through 70 μM Nylon strainer. Cells suspensions were pelleted, resuspended in complete MEM medium, and then cultured in T-75 flasks until the cell layer becomes confluent.

Multi-step growth in primary chicken embryo fibroblasts (CEFs). 5×10⁵ CEF cells were seeded in a 6-well plate and were cultured overnight. Cells were infected with either MVA, MVAΔC7L-hFlt3L, MVAΔC7L-hFlt3L-TK(−)-mOX40L, or MVAΔC7L-hFlt3L-TK(−)-hOX40L at a MOI of 0.05 for one hour. The inoculum was removed and cells were washed with PBS once and incubated with fresh medium. Cells were collected at 1, 24, 48, and 72 h post infection. After three cycles of freezing and thawing, the samples were sonicated and virus titers were determined by serial dilution and infection of BHK21 cells. Confocal microscope was used to visualize GFP⁺ foci for counting.

Generation of human monocyte-derived dendritic cells. All collection and use of human specimens adhered to protocols reviewed and approved by the Institutional Review and Privacy Board of Memorial Hospital, MSKCC. Buffy coats were obtained from healthy donors at New York Blood Center and peripheral blood mononuclear cells (PBMCs) separated by standard centrifugation over Ficoll-Paque PLUS (Amersham Pharmacia Biotech, Uppsala, Sweden). Tissue culture plastic adherent PBMCs comprised the moDCs precursors, which were cultured in complete RPMI 1640-1% human serum supplemented with GM-CSF (1000 IU/ml) and IL-4 (500 IU/ml). Fresh medium and cytokines were replenished every 48 h. Immature (day 6) moDCs were infected with Heat-iMVA, MVAΔC7L-hFlt3L-TK(−), or MVAΔC7L-hFlt3L-TK(−)-hOX40L at a MOI of 1, or treated with poly I:C at 10 μg/ml. Cells were collected at 24 h post treatment and stained with PE-conjugated anti-hOX40L antibody prior to FACS analyses.

Dual Luciferase Reporter assay. Luciferase activities were measured using the Dual Luciferase Reporter Assay system according to the manufacturer's instructions (Promega). Briefly, expression plasmids including a firefly luciferase reporter construct, a Renilla luciferase reporter construct, as well as other expression constructs were transfected into HEK293T cells. Murine cGAS (50 ng) and hSTING (10 ng) were used at suboptimal dosages for the purpose of identifying inhibitors. The transfected plasmids containing viral genes were used at 200 ng. IFNB-firefly luciferase reporter and control plasmid pRL-TK were used at 50 ng and 10 ng, respectively. 24 h post transfection, cells were collected and lysed. 20 μl cell lysates were incubated with 50 μl of LARII to measure firefly luciferase activity and then were incubated with 50 μl of Stop & Glo Reagent to measure Renilla luciferase activity. The relative luciferase activity was expressed as arbitrary units by normalizing firefly luciferase activity under IFNB or ISRE promoter to Renilla luciferase activity from a control plasmid pRL-TK. Fold-induction was calculated by dividing relative luciferase activity under a certain test condition by that under background condition.

Generation of retrovirus expressing vaccinia E5, K7, B14, B18. HEK293T cells were passaged into a 6-well plate. The next day, cells were transfected with three plasmids: VSVG (1 μg); gag/pol (2 μg); and PQCXIP-E5, K7, B14, B18 (2 g), with 10 μl lipofectamine 2000. After 2 days, cell supernatants were collected and filtered through a 0.45 m filter and stored in −80° C.

Generation of RAW264.7 cell line stably expressing vaccinia FLAG-tagged E5, K7, B14, or B18. RAW264.7 cells were passaged into a 6-well plate. The next day, cells were infected with retrovirus expressing E5, K7, B14, or B18 at MOI 5. After 2 days, culture medium was replaced with fresh DMEM medium containing 5 g/ml puromycin. After one week, survival cells are the cells stably expressing FLAG-tagged E5, K7, B14, or B18. The expression of FLAG-tagged E5, K7, B14, or B18 was verified by Western blot analysis using anti-FLAG antibody.

RNA isolation and quantitative real-time PCR. RNA was extracted from whole-cell lysates with an RNeasy Mini kit (Qiagen) and was reverse transcribed with a First Strand cDNA synthesis kit (Fermentas). Quantitative real-time PCR was performed in triplicate with SYBR Green PCR Mater Mix (Life Technologies) and Applied Biosystems 7500 Real-time PCR Instrument (Life Technologies) using gene-specific primers. Relative expression was normalized to the levels of glyceraldehyde-3-phosphate dehydrogenase (GAPDH).

Reagents. The commercial sources for reagents were as follows: anti-hFlt3L antibody was purchased from Thermo Fisher and secondary antibody for anti-hFlt3L (PE-conjugated goat anti-mouse) was from BD Biosciences. PE-conjugated mOX40L and PE-conjugated anti-hOX40L antibody were purchased from Biosciences and R & D respectively. Anti-CD3, -CD45, -CD8, and -Granzyme B antibodies were purchased form eBioscience (Thermo Fisher Scientific, Waltham, MA). CD8a microbeads was from Miltenyi Biotechnology (Somerville, MA). ELISPOT assay kit was purchased from Becton-Dickinson Biosciences (Franklin Lakes, NJ). Therapeutic anti-CTLA4 (clone 9H10 and 9D9), anti-PD1 (clone RMP1-14), anti-PD-L1 (clone 10F.9G2) were purchased from BioXcell; Antibodies used for flow cytometry were purchased from eBioscience (CD45.2 Alexa Fluor 700, CD3 PE-Cy7, CD4 APC-efluor780, CD8 PerCP-efluor710), Invitrogen (CD4 QDot 605, Granzyme B PE-Texas Red, Granzyme B APC).

Statistics. Two-tailed unpaired Student's t test was used for comparisons of two groups in the studies. Survival data were analyzed by log-rank (Mantel-Cox) test. The p values deemed significant are indicated in the figures as follows: *, P<0.05; **, P<0.01; ***, P<0.001; ****, P<0.0001.

Generation of B2M, MDA5, STING knock-out cell lines. B16-F10 cells were transfected with 800 ng of Cas9 expression plasmid (obtained from Church lab through Addgene) and 800 ng of each gRNA expression plasmid using Lipofectamine 3000 reagent (Thermo Scientific). Cells were allowed to grow for at least 2 days after transfection. The success of CRISPR constructs was then tested. In the case of STING and MDA5 CRISPR, targeted exons were PCR-amplified from genome with specific primers and then digested with 2 units of T7 endonuclease (obtained from new England biolabs) for 90 minutes. After digestion, agarose gel electrophoresis was performed to determine whether T7 cleaved the PCR amplicons, indicating successful CRISPR. After confirmation of gRNA efficacy, individual cells were seeded onto 96-well plates and expanded into clonal isolates. After expansion, clonal isolates were screened using another round of PCR amplification and T7 digestion. Subsequent Western Blot analysis confirmed loss of targeted proteins (STING or MDA5). In the case of Beta 2 Microglobulin (B2M) CRISPR, FACS analysis was used to verify successful CRISPR before sorting of B2M deficient cells onto 96 well plates and expanded into clonal isolates.

Human tumor tissues from patients with Extramammary Paget Disease (EMPD). Human tumor tissues were obtained from patients with Extramammary Paget disease (EMPD) enrolled in IRB-approved clinical protocol 06-107 at Memorial Sloan Kettering Cancer Center. 3-4 mm punch biopsy was performed by the clinician in the clinic. The tumor tissues were transported to the laboratory in RPMI medium on ice. Once they arrived in the lab, they were cut into small pieces with a scalpel and infected with MVAΔE5R-hFlt3L-hOX40L at a MOI of 10. 48 h post infection, tissues were digested with collagenase D at 37° C. for 45 min. Then they were filtered and stained with surface antibody for CD3, CD4, and CD8. After that, they were permeabilized and stained with antibodies for Granzyme B and Foxp3.

### Example 1: Generation of Recombinant MVAΔE3L with a TK-Deletion Expressing Murine OX40L

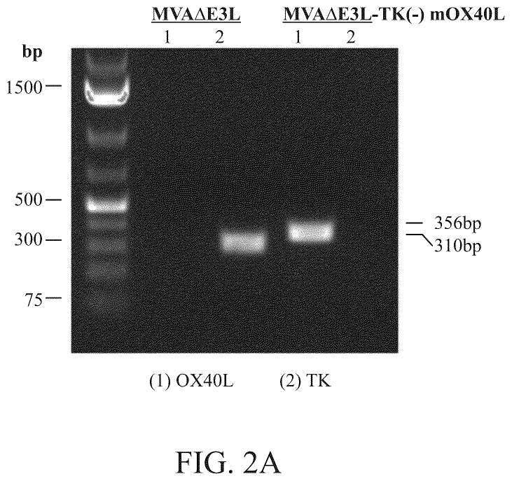

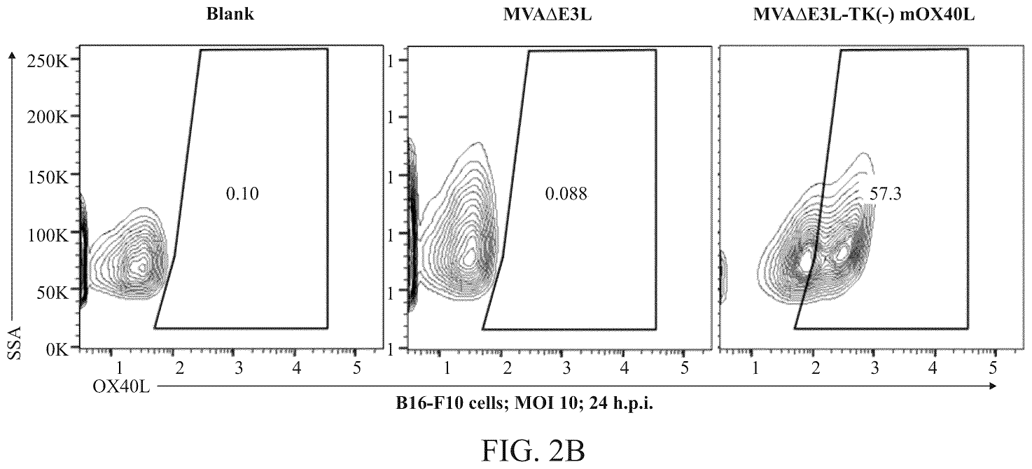

This example describes the generation of a recombinant vaccinia MVAΔE3L virus comprising a TK-deletion expressing murine OX40L (mOX40L). FIG. 1 shows the schematic diagram of an expression cassette designed to express mOX40L using the vaccinia viral synthetic early and late promoter (PsE/L). A plasmid containing a codon optimized mOX40L gene under the control of the vaccinia PsE/L as well as the E. coli xanthine-guanine phosphoribosyl transferase gene (gpt) under the control of vaccinia P7.5 promoter flanked by the thymidine kinase (TK) gene on either side was constructed using standard recombinant virus technology through homologous recombination at the TK locus between pCB plasmid DNA and viral genomic DNA (FIG. 1). BHK21 cells were infected with MVAΔE3L at a multiplicity of infection (MOI) of 0.5) for 1 h, and then were transfected with the plasmid DNAs described above. The infected cells were collected at 48 h. Recombinant viruses were selected through further culturing in gpt selection medium including MPA, xanthine and hypoxanthine, and plaque purified. PCR analysis was performed to verify that MVAΔE3L-TK(−)-mOX40L lacks part of the TK gene and with mOX40L insertion (FIG. 2A). The expression of mOX40L on B16-F10 cells infected with MVAΔE3L-TK(−)-mOX40L virus was determined by FACS analysis using anti-mOX40L antibody (FIG. 2B). Briefly, B16-F10 cells were infected with MVAΔE3L-TK(−)-mOX40L at a MOI of 10. At 24 h post infection, cells were stained with PE-conjugated anti-mOX40L antibody. The expression of mOX40L on the surface of infected cells were evaluated by FACS analysis. FIG. 2B shows that the majority of the infected cells express mOX40L.

### Example 2: Intratumoral Injection of MVAΔE3L-TK(−)-mOX40L Results in More Activated Tumor-Infiltrating Effector T Cells in Distant Tumors Compared with MVAΔE3L in B16-F10 Bilateral Tumor Implantation Model

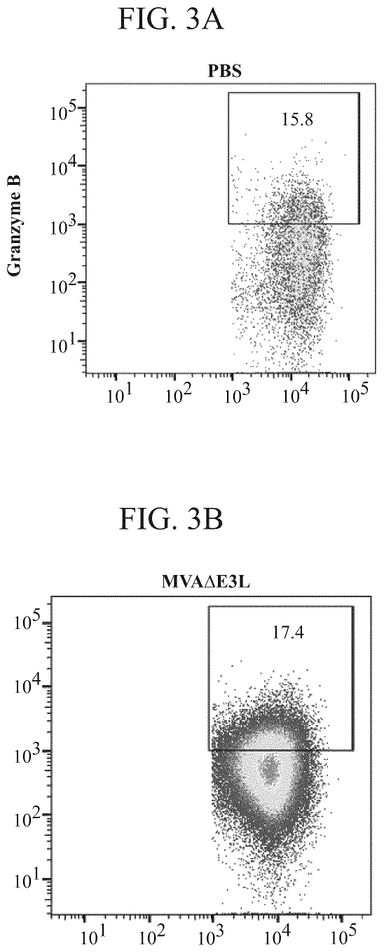

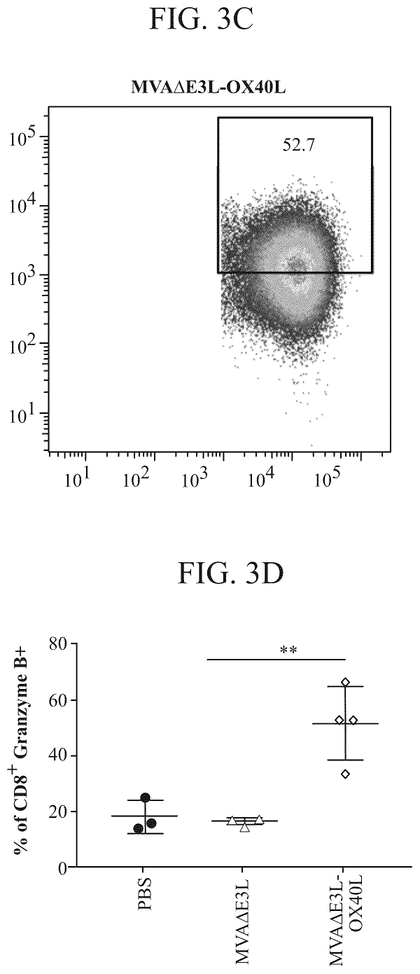

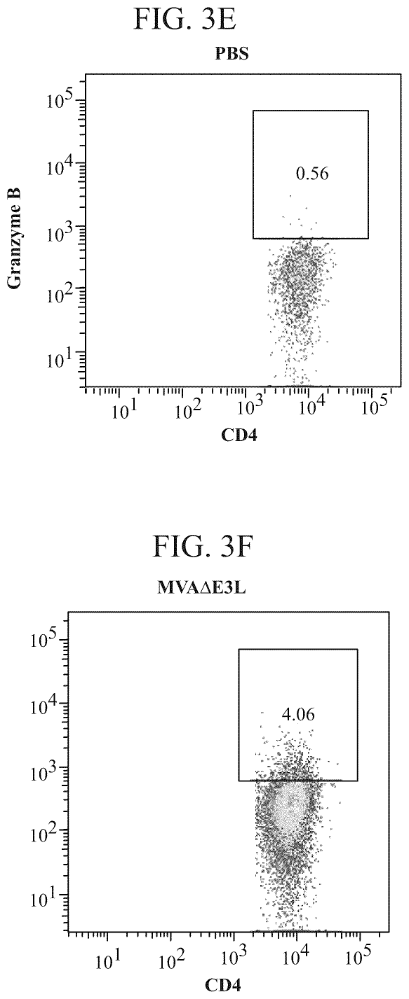

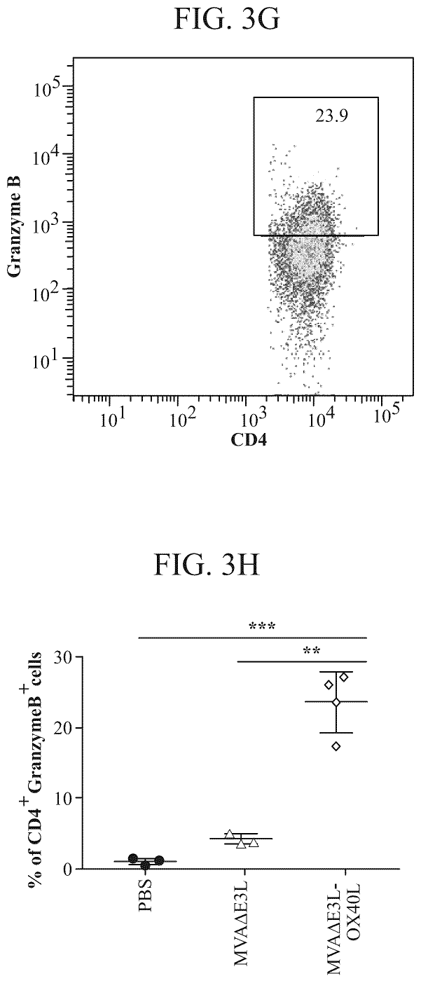

To assess whether intratumoral injection of MVAΔE3L-TK(−)-mOX40L or MVAΔE3L in B16-F10 melanomas leads to activation and proliferation of CD8⁺ and CD4⁺ T cells, a bilateral tumor implantation model was used. B16-F10 melanoma cells were implanted intradermally into the shaved skin on the right (5×10⁵ cells) and left (2.5×10⁵ cells) flanks of a C57BL/6J mouse. After 7 days post implantation, the larger tumors on the right flank (about 3 mm or larger in diameter) were injected twice per week with PBS, MVAΔE3L, MVAΔE3L-TK(−)-mOX40L when the mice were under anesthesia. Three days after the second injection, tumors were harvested and cells were processed for surface labeling with anti-CD3, CD45, CD4, and CD8 antibodies, and also for intracellular Granzyme B staining. The live immune cell infiltrates in the non-injected tumors were analyzed by FACS. There was a dramatic increase in CD8⁺ T cells expressing Granzyme B in the non-injected tumors, from 18% in tumors of PBS-treated mice and 17% in tumors of MVAΔE3L-treated mice to 53% in the tumors of MVAΔE3L-TK(−)-mOX40L-treated mice (FIG. 3A-D). There was also a significant increase in CD4⁺ T cells expressing Granzyme B in the non-injected tumors after intratumoral virus treatment, from 0.6% in tumors of PBS-treated mice to 4.3% in MVAΔE3L-treated mice to 24% in the tumors of MVAΔE3L-TK(−)-mOX40L-treated mice (FIG. 3E-H). These results demonstrate that intratumoral injection of the recombinant MVAΔE3L-TK(−)-mOX40L is more potent than its parental virus MVAΔE3L in inducing cytotoxic CD8⁺ T cells and/or CD4⁺ T cells within non-injected tumors.

### Example 3: Intratumoral Injection with MVAΔE3L-TK(−)-mOX40L Leads to the Generation of Systemic Antitumor CD8⁺ T-Cell Immunity

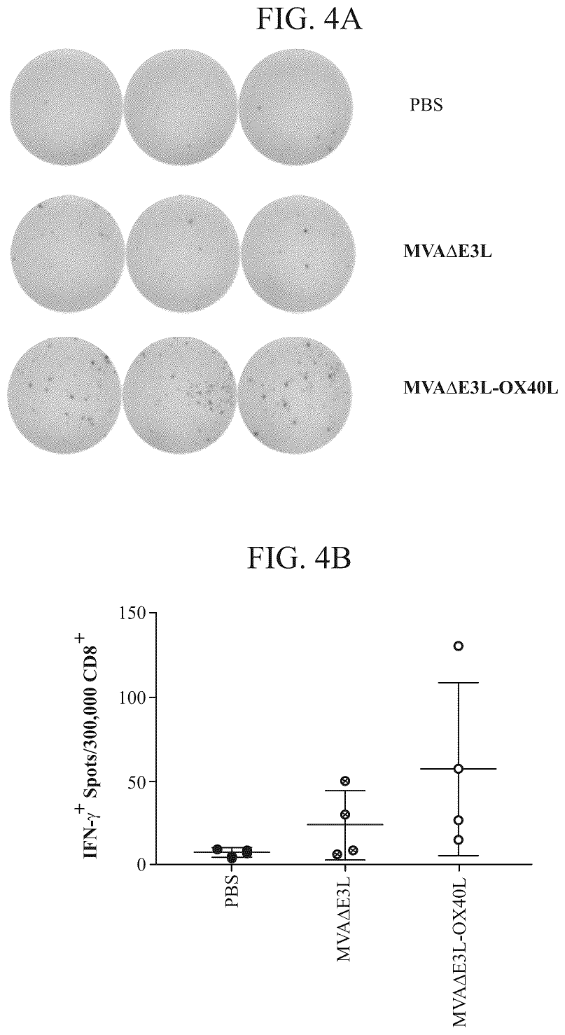

To assess whether mice gained systemic antitumor T-cell immunity against the murine B16-F10 melanoma cancer after treatment with intratumoral injection of MVAΔE3L-TK(−)-mOX40L or MVAΔE3L, Enzyme-linked ImmunoSpot (ELISpot) was used. B16-F10 cells (5×10⁵ and 2.5×10⁵, respectively) were intradermally implanted into the shaved skin on the right and left flank of C57BL/6J mice. Seven days after tumor implantation the tumors on the right flank (about 3 mm in diameter) were injected with PBS, MVAΔE3L, or MVAΔE3L-TK(−)-mOX40L. The injections were repeated three days later, followed by euthanization three days after the second injection. ELISpot was performed to assess the generation of antitumor specific CD8⁺ T cells in the spleens of mice treated with the recombinant viruses. Briefly, CD8⁺ T cells were isolated from splenocytes and 3×10⁵ cells were cultured with 1.5×10⁵ irradiated B16-F10 cells overnight at 37° C. in anti-IFN-γ-coated BD ELISpot plate microwells. CD8⁺ T cells were stimulated with B16-F10 cells irradiated with an γ-irradiator and IFN-γ secretion was detected with an anti-IFN-γ antibody. FIG. 4A shows representative images of IFN-γ⁺ spots per 300,000 CD8⁺ T cells from individual mouse treated with either PBS, MVAΔE3L, or MVAΔE3L-TK(−)-mOX40L. FIG. 4B shows the numbers of IFN-γ⁺ spots per 300,000 CD8⁺ T cells from individual mouse in each group treated with either PBS, MVAΔE3L, or MVAΔE3L-TK(−)-mOX40L. These results demonstrate that intratumoral injection of MVAΔE3L-TK(−)-mOX40L is more effective than MVAΔE3L in generating antitumor CD8⁺ T cells in treated mice in a murine B16-F10 melanoma bilateral implantation model. Accordingly, these results demonstrate that the recombinant MVAΔE3L-TK(−)-mOX40L of the present technology are effective in enhancing or promoting an immune response in the subject and in increased cytotoxic CD8⁺ T cells within of a subject.

### Example 4: Generation of Recombinant MVAΔC7L with a TK-Deletion Expressing Murine OX40L

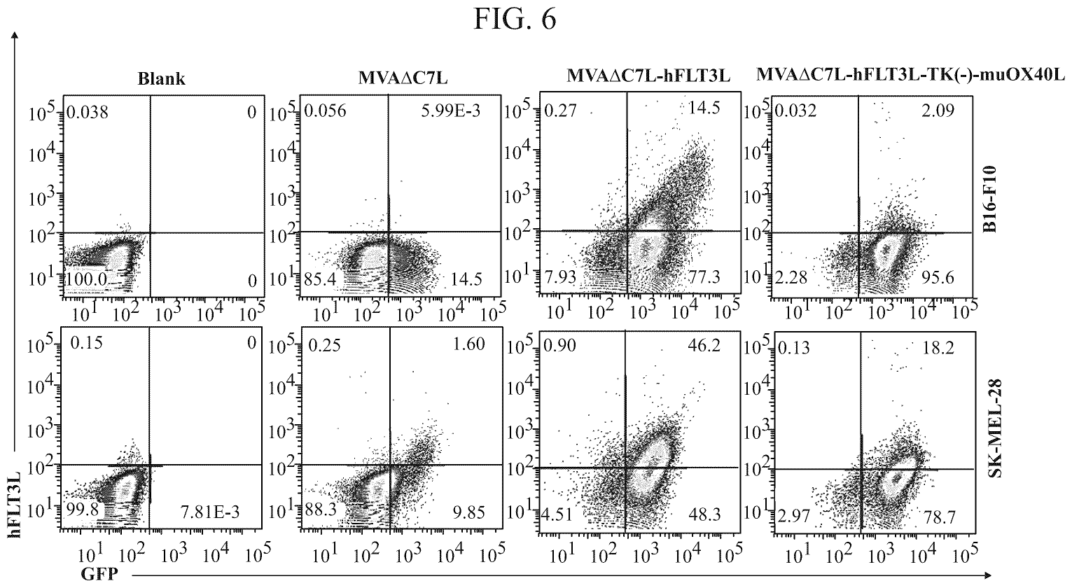

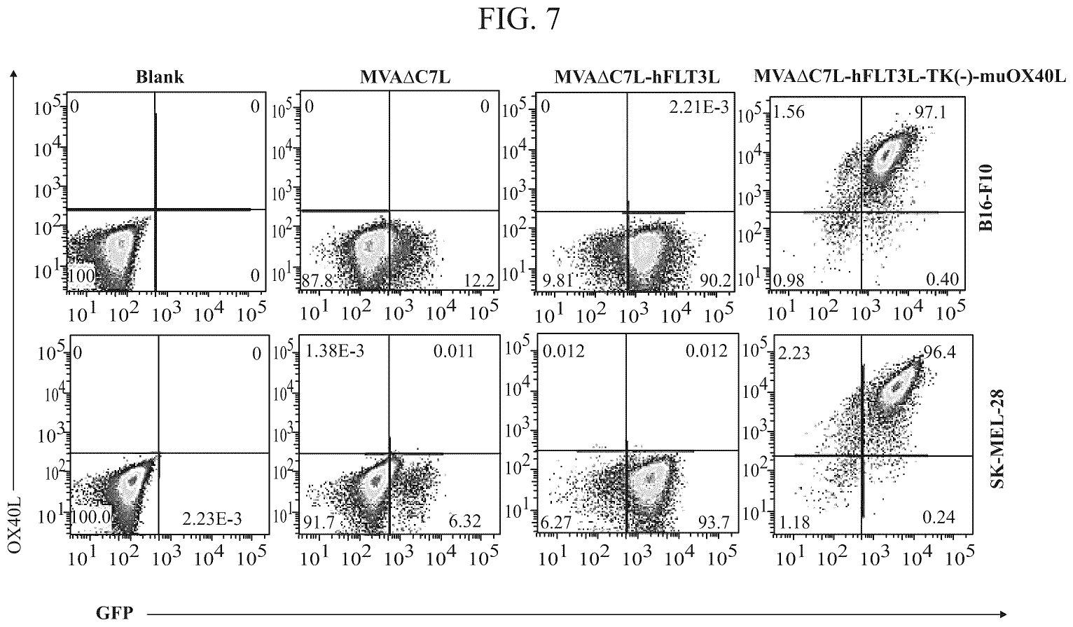

This example describes the generation of a recombinant vaccinia MVAΔC7L virus comprising a TK-deletion expressing murine OX40L (mOX40L). FIG. 5A shows the first step to generate MVAΔC7L-hFlt3L. pUC57 vector was constructed to insert a specific gene of interest (SG) into the C7L locus of MVA, which includes an expression cassette designed to express hFlt3L using the vaccinia viral synthetic early and late promoter (PsE/L) and GFP under the control of the vaccinia P7.5 promoter used as a selection marker. The expression cassette was flanked by partial sequence of C8L and C6R on the left and right side of C7L gene (FIG. 5A). BHK21 cells were infected with MVA at a multiplicity of infection (MOI) of 0.5 for 1 h, and then were transfected with the plasmid DNAs described above. The infected cells were collected at 48 h. Recombinant viruses were selected by serial selection of GFP⁺ foci. PCR analysis was performed to verify that MVAΔC7L-hFlt3L lacks the C7L gene and with hFlt3L insertion (data not shown). FIG. 5B shows the second step to generate MVAΔC7L-hFlt3L-TK(−)-mOX40L. A plasmid containing a codon optimized mOX40L gene under the control of the vaccinia PsE/L as well as the E. coli xanthine-guanine phosphoribosyl transferase gene (gpt) under the control of vaccinia P7.5 promoter flanked by the thymidine kinase (TK) gene on either side was constructed. BHK21 cells were infected with MVAΔC7L-hFlt3L at a multiplicity of infection (MOI) of 0.5 for 1 h, and then were transfected with the plasmid DNAs described above. The infected cells were collected at 48 h. Recombinant viruses were selected through further culturing in gpt selection medium including MPA, xanthine and hypoxanthine, and plaque purified. PCR analysis was performed to verify that MVAΔC7L-hFlt3L-TK(−)-mOX40L lacks C7L gene and part of the TK gene, but with both hFlt3L and mOX40L insertion. By contrast, the parental MVA genome contains C7L and TK gene as expected (FIG. 5C). The expression of mOX40L on murine B16-F10 cells and human SK-MEL-28 cells infected with MVAΔC7L-TK(−)-mOX40L virus was determined by FACS analysis using anti-hFlt3L antibody (FIG. 6) and anti-mOX40L antibody (FIG. 7). The majority of both murine B16-F10 and SK-MEL28 cells had high expression levels of mOX40L (FIG. 7).

### Example 5: Intratumoral (IT) Injection of MVAΔC7L-hFlt3L-TK(−)-mOX40L Leads to Recruitment of Activated CD8⁺ and CD4⁺ T Cells into the Non-Injected Distant Tumors in B16-F10 Bilateral Tumor Implantation Model

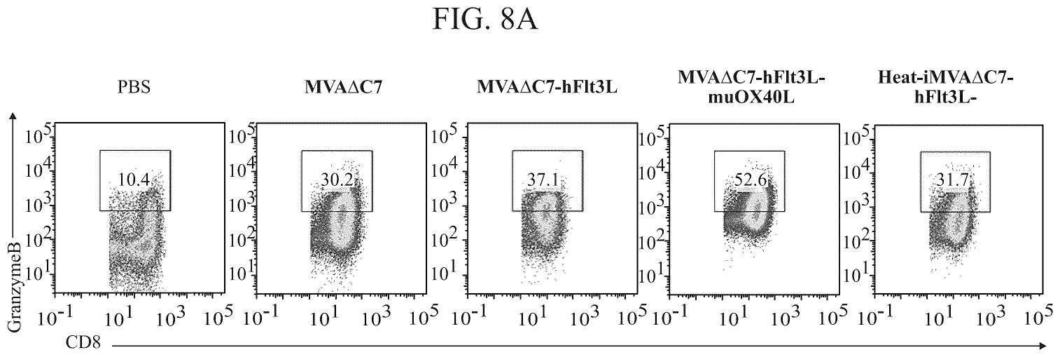

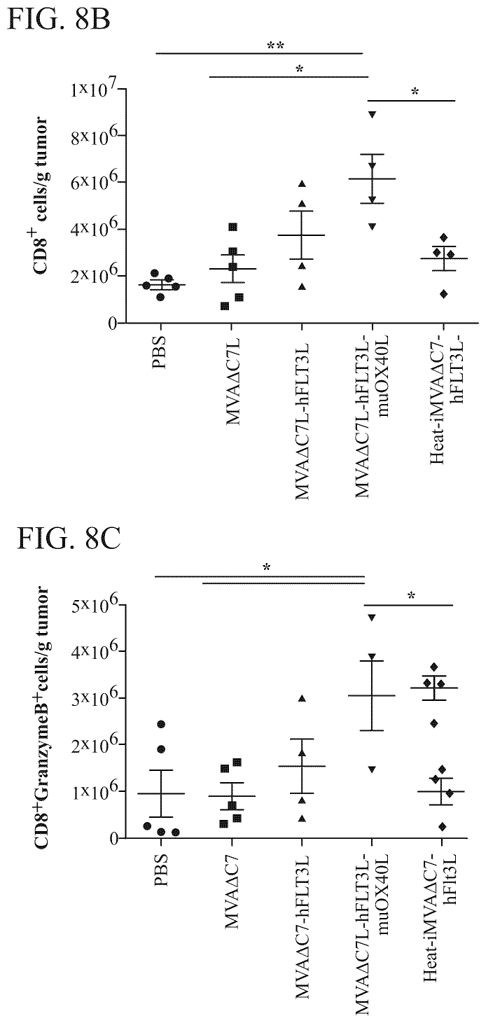

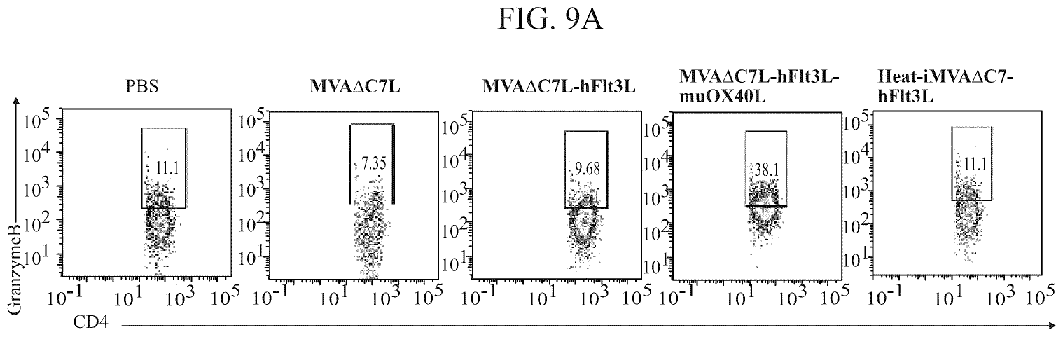

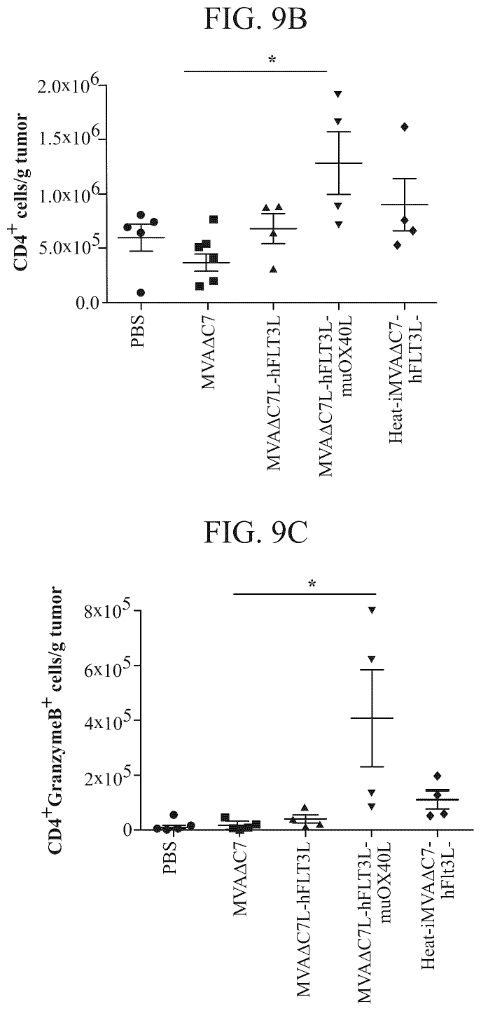

To assess whether IT MVAΔC7L-hFlt3L-TK(−)-mOX40L results in the generation of systemic antitumor immunity, a bilateral B16-F10 tumor implantation model was used. Briefly, B16-F10 melanoma cells were implanted intradermally into the shaved skin on the right (5×10⁵ cells) and left (2.5×10⁵ cells) flanks of a C57BL/6J mouse. After 7 days post implantation, the larger tumors on the right flank were injected twice per week with PBS, MVAΔC7L, MVAΔC7L-hFlt3L, or MVAΔC7L-hFlt3L-TK(−)-mOX40L at a MOI of 2×10⁷ pfu, or with an equivalent amount of Heat-inactivated MVAΔC7L-hFlt3L (Heat-iMVAΔC7L-hFlt3L). Two days after the second injection, tumors were harvested and cells were processed for surface labeling with anti-CD3, CD45, CD4, and CD8 antibodies, and also for intracellular Granzyme B staining. The live immune cell infiltrates in the non-injected tumors were analyzed by FACS. IT MVAΔC7L-hFlt3L-TK(−)-mOX40L resulted in the highest number of total CD8⁺ T cells per gram of tumor as well as total Granzyme B⁺ CD8⁺ T cells per gram of tumor in the non-injected tumors compared with the other treatment groups (FIGS. 8A-8C). Remarkably, IT MVAΔC7L-hFlt3L-TK(−)-mOX40L resulted in the highest number of total CD4⁺ T cells per gram of tumor as well as total Granzyme B⁺ CD4⁺ T cells per gram of tumor in the non-injected tumors compared with the other treatment groups (FIGS. 9A-9C). These results demonstrate that IT MVAΔC7L-hFlt3L-TK(−)-mOX40L is more effective than its parental virus MVAΔC7L or MVAΔC7L-hFlt3L in inducing cytotoxic CD8⁺ T cells and CD4⁺ T cells within non-injected tumors. In addition, this engineered recombinant MVA is more potent than the inactivated MVAΔC7L-hFlt3L in inducing anti-tumor CD8⁺ and CD4⁺ T cell responses.

### Example 6: IT Injection of MVAΔC7L-hFlt3L-TK(−)-mOX40L Leads to the Strongest Systemic Antitumor CD8⁺ T-Cell Immunity Compared with MVAΔC7L

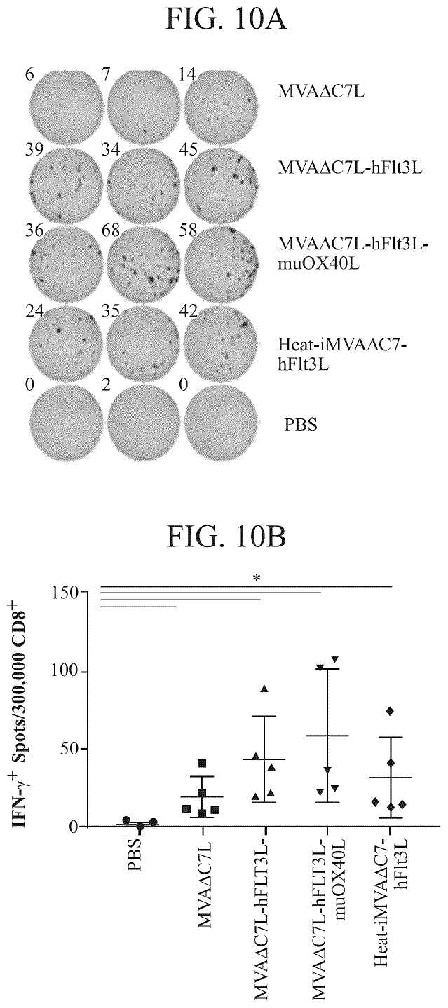

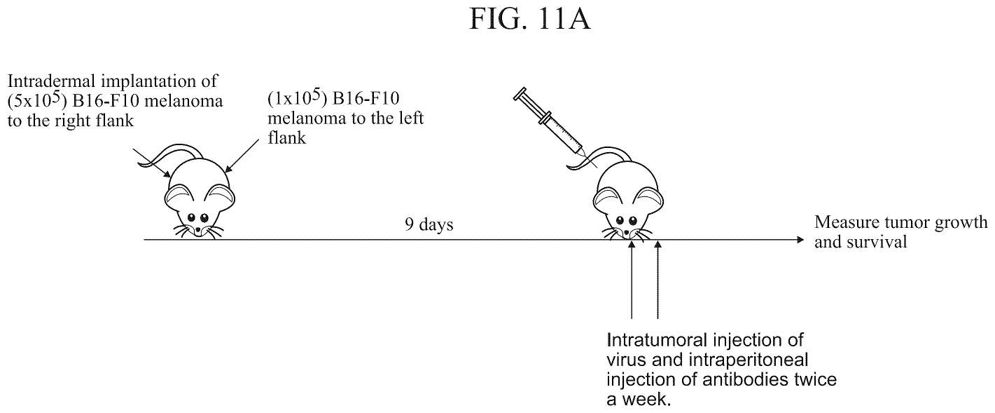

ELISpot was performed to assess the generation of antitumor specific CD8⁺ T cells in the spleens of mice treated with the recombinant viruses as described in Example 5. Briefly, CD8⁺ T cells were isolated from splenocytes and 3×10⁵ cells were cultured with 1.5×10⁵ irradiated B16-F10 cells overnight at 37° C. in anti-IFN-γ-coated BD ELISpot plate microwells. CD8⁺ T cells were stimulated with B16-F10 cells irradiated with an γ-irradiator and IFN-γ secretion was detected with an anti-IFN-γ antibody. FIG. 10A shows representative images of IFN-γ⁺ spots per 300,000 CD8⁺ T cells from individual mouse treated with either PBS, MVAΔC7L, MVAΔC7L-hFlt3L, MVAΔC7L-hFlt3L-TK(−)-mOX40L, or Heat-iMVAΔC7L. FIG. 10B shows the numbers of IFN-γ⁺ spots per 300,000 CD8⁺ T cells from individual mouse in each group treated with either PBS, MVAΔC7L, MVAΔC7L-hFlt3L, MVAΔC7L-hFlt3L-TK(−)-mOX40L, or Heat-iMVAΔC7L. These results demonstrate that IT injection of MVAΔC7L-hFlt3L-TK(−)-mOX40L is more effective than MVAΔC7L in generating antitumor CD8⁺ T cells in treated mice in a murine B16-F10 melanoma bilateral implantation model.

### Example 7: IT Injection of MVAΔC7L-hFlt3L-TK(−)-mOX40L is Effective in Eradicating or Delaying Tumor Growth of Both Injected and Non-Injected Tumors and Prolonging Survival of Mice

To test whether IT delivery of MVAΔC7L-hFlt3L-TK(−)-mOX40L generates antitumor effects, a bilateral murine B16-F10 tumor implantation model was used. Briefly, B16-F10 melanoma cells were implanted intradermally to the left and right flanks of C57B/6 mice (5×10⁵ to the right flank and 1×10⁵ to the left flank). Nine days after tumor implantation, MVAΔC7L-hFlt3L-TK(−)-mOX40L (2×10⁷ PFU) was delivered into the larger tumors on the right flank twice weekly, with concomitant intraperitoneal (IP) injection of with either anti-CTLA-4 antibody (9D9 clone, 100 μg per mouse), or anti-PD-L1 (250 μg per mouse), or isotype control. Tumor sizes were measured twice a week and mice survival were monitored (FIG. 11A). The volumes of injected and non-injected tumors of individual mouse are shown in FIG. 11B and FIG. 11C. The volumes of injected and non-injected tumors at Day 0, 7, and 11 are shown in FIG. 11D and FIG. 11E. In mice treated with PBS, tumors grew rapidly, which resulted in early death with a median survival of 7 days (FIGS. 11F and 11G). Intratumoral injection of MVAΔC7L-hFlt3L-TK(−)-mOX40L resulted in delayed tumor growth and improved survival compared with PBS, with an extension of median survival to 14 days (FIGS. 11F and 11G). B16-F10 melanoma is a very aggressive tumor. The inventors intentionally waited for 9 days after tumor implantation before starting treatment.

### Example 8: The Combination of IT Injection of MVAΔC7L-hFlt3L-TK(−)-mOX40L with Systemic Delivery of Immune Checkpoint Blockade Generates Synergistic Anti-Tumor Responses

The combination with IT delivery of MVAΔC7L-hFlt3L-TK(−)-mOX40L and systemic delivery of anti-CTLA-4 or anti-PD-L1 had superior anti-tumor efficacy compared with IT virus alone. The average tumor volumes of both injected and non-injected tumors were smaller in the IT MVAΔC7L-hFlt3L-TK(−)-mOX40L plus anti-PD-L1 group, followed by IT MVAΔC7L-hFlt3L-TK(−)-mOX40L plus anti-CTLA-4 group, when compared with IT virus alone or PBS mock treatment (FIGS. 11B-11E). The median survival of mice was extended to 21 days in the IT MVAΔC7L-hFlt3L-TK(−)-mOX40L plus anti-CTLA-4 group, and to 26.5 days in the IT MVAΔC7L-hFlt3L-TK(−)-mOX40L plus anti-PD-L1 (FIGS. 11F and 11G). This result demonstrates that anti-tumor effects induced by IT MVAΔC7L-hFlt3L-TK(−)-mOX40L can be enhanced in the presence of immune checkpoint blockade in a bilateral murine tumor model. In this B16-F10 tumor model, the immune checkpoint blockade antibody alone is not effective, which has been shown by many laboratories including the inventors' laboratories.

### Example 9: The Combination of IT Injection of MVAΔC7L-hFlt3L-TK(−)-mOX40L with Systemic Delivery of Immune Checkpoint Blockade is More Effective than IT Virus Alone in Shrinking and Eradicating Large Established B16-F10 Melanoma

To test whether the combination with IT delivery of MVAΔC7L-hFlt3L-TK(−)-mOX40L and systemic delivery of anti-CTLA-4, anti-PD-1, or anti-PD-L1 had superior anti-tumor efficacy compared with IT virus alone against large established B16-F10 melanoma, 5×10⁵ cells were intradermally implanted into the right flanks of C57B/6 mice. Nine days after tumor implantation, when the tumors were 5 mm in diameter, they were treated with either IT PBS, MVAΔC7L-hFlt3L-TK(−)-mOX40L alone, MVAΔC7L-hFlt3L-TK(−)-mOX40L plus IP anti-CTLA-4, MVAΔC7L-hFlt3L-TK(−)-mOX40L plus IP anti-PD-1, or MVAΔC7L-hFlt3L-TK(−)-mOX40L plus IP anti-PD-L1 twice weekly. Tumor volumes were measured and mice survival was monitored. Although IT MVAΔC7L-hFlt3L-TK(−)-mOX40L alone has some anti-tumor effects, the combination of IT virus with anti-PD-1 had the best synergistic effects, followed by the combinations of IT virus plus anti-PD-L1, and IT virus plus anti-CTLA-4.

### Example 10: Generation of Recombinant MVAΔC7L-hFlt3 with a TK-Deletion Expressing Human OX40L

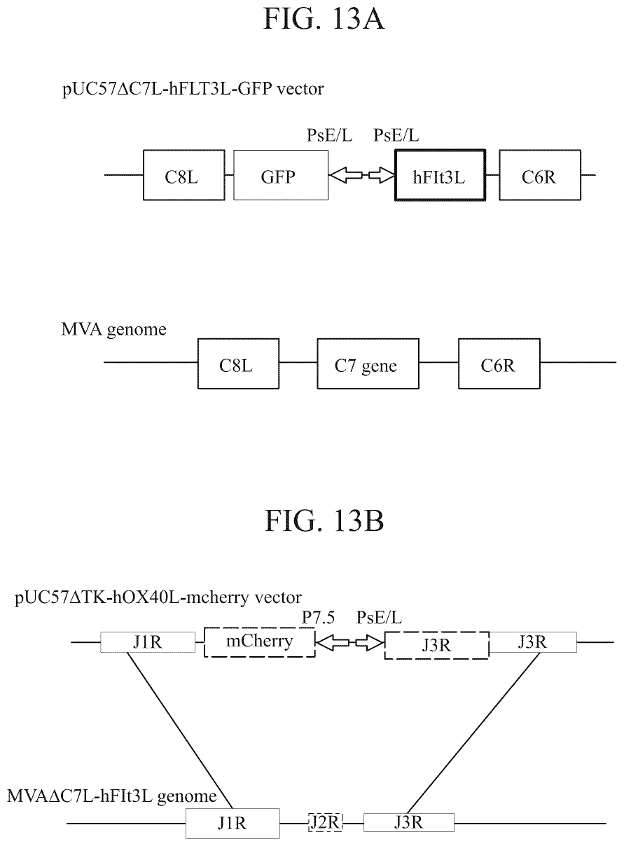

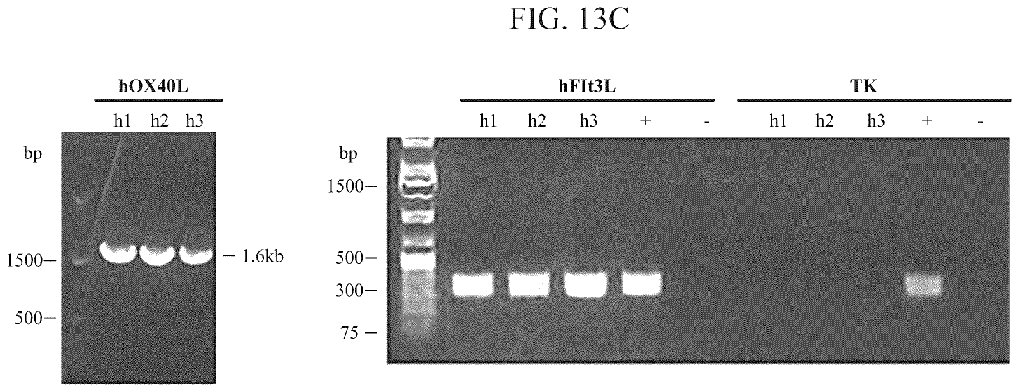

This example describes the generation of a recombinant vaccinia MVAΔC7L-hFlt3L virus comprising a TK-deletion expressing human OX40L (hOX40L). FIG. 13A shows the first step to generate MVAΔC7L-hFlt3L, which has been described in Example 4. FIG. 13B shows the second step to generate MVAΔC7L-hFlt3L-TK(−)-hOX40L. A plasmid containing human OX40L gene under the control of the vaccinia PsE/L as well as the mCherry under the control of vaccinia P7.5 promoter flanked by the thymidine kinase (TK) gene on either side was constructed. BHK21 cells were infected with MVAΔC7L-hFlt3L at a multiplicity of infection (MOI) of 0.5 for 1 h, and then were transfected with the plasmid DNAs described above. The infected cells were collected at 48 h. Recombinant viruses were selected through picking mCherry foci, and plaque purified. PCR analysis was performed to verify that MVAΔC7L-hFlt3L-TK(−)-hOX40L lacks C7L gene and part of the TK gene, but with both hFlt3L and hOX40L insertion (data not shown). The insert was also sequenced to verify the accuracy of the sequences.

### Example 11: The Recombinant Viruses MVAΔC7L-hFlt3L-TK(−)-mOX40L and MVAΔC7L-hFlt3L-TK(−)-hOX40L Replicate Efficiently in Chicken Embryo Fibroblasts

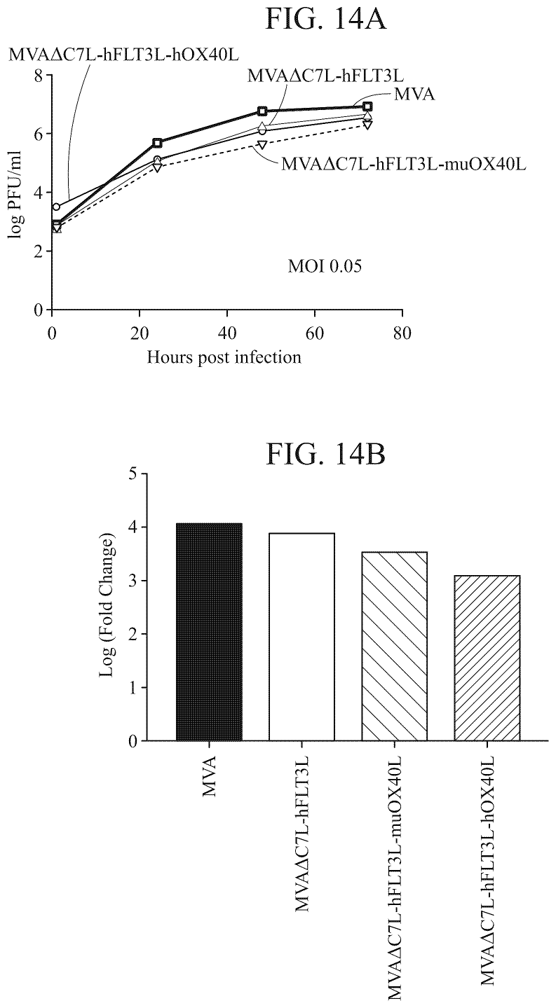

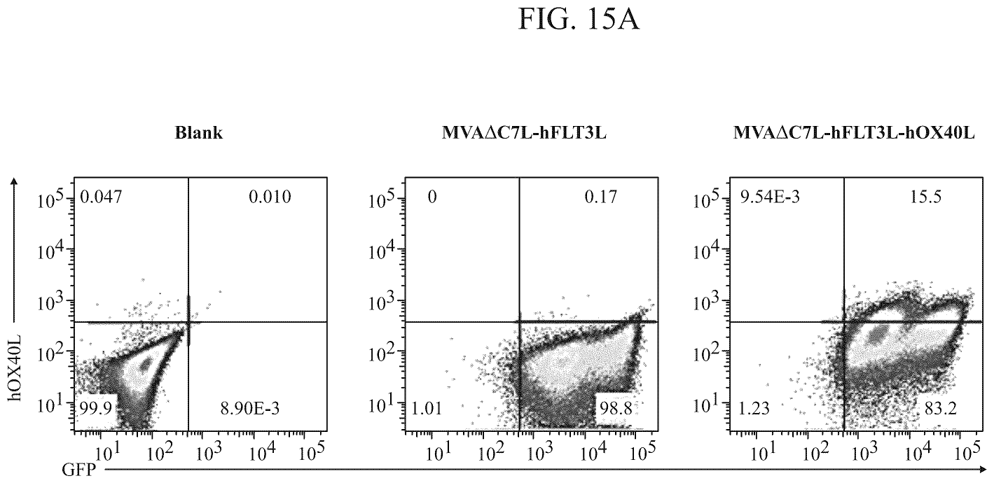

MVA is commonly manufactured in chicken embryo fibroblasts (CEFs). To test whether the recombinant MVA viruses, MVAΔC7L-hFlt3L-TK(−)-mOX40L and MVAΔC7L-hFlt3L-TK(−)-hOX40L replicate in CEFs, a multi-step replication assay was performed. 5×10⁵ CEF cells were seeded in a 6-well plate and were cultured overnight. Cells were infected with either MVA, MVAΔC7L-hFlt3L, MVAΔC7L-hFlt3L-TK(−)-mOX40L, or MVAΔC7L-hFlt3L-TK(−)-hOX40L at a MOI of 0.05 for one hour. The inoculum was removed and cells were washed with PBS once and incubated with fresh medium. Cells were collected at 1, 24, 48, and 72 h post infection. Viral titers were determined on BHK21 cells. FIG. 14A shows viral titers of MVA, MVAΔC7L-hFlt3L, MVAΔC7L-hFlt3L-TK(−)-mOX40L, or MVAΔC7L-hFlt3L-TK(−)-hOX40L over time after infection in CEFs. All of the viruses replicate efficiently in CEFs, with increase of titers over 1,000 to 10,000-fold (FIG. 14B). The recombinant MVA viruses have slight reduction of viral titers compared with the parental MVA (FIGS. 14A and 14B).

### Example 12: The Recombinant Viruses MVAΔC7L-hFlt3L-TK(−)-hOX40L Express hOX40L Protein on the Surface of Infected Cells

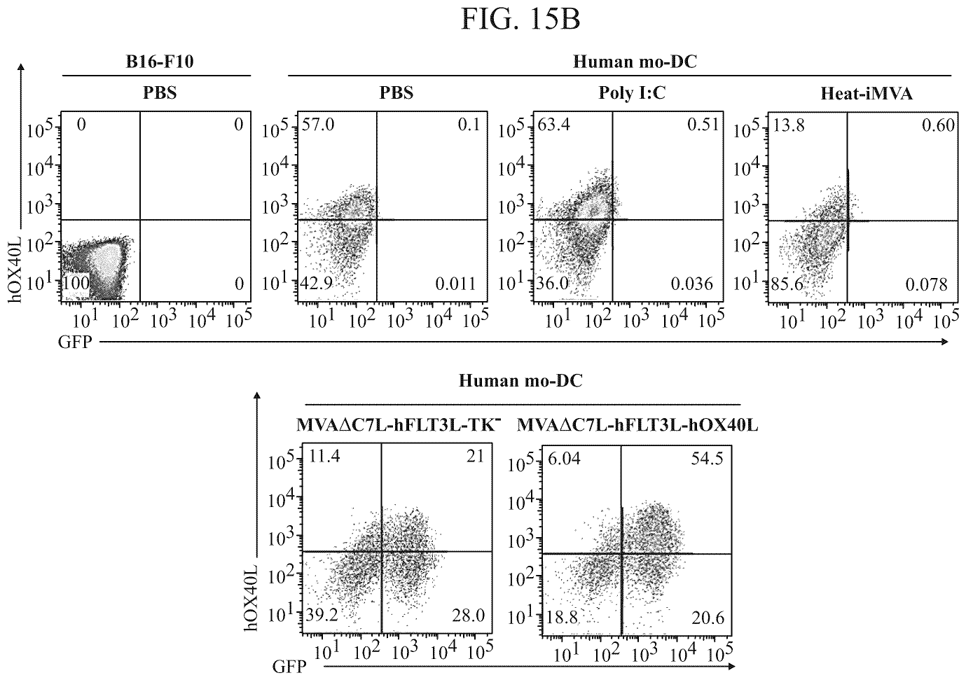

FACS analysis was used to determine the expression of hOX40L on the surface of infected cells. Briefly, BHK21 cells were infected either with MVAΔC7L-hFlt3L or MVAΔC7L-TK(−)-hOX40L at a MOI of 10. At 24 h post infection, cells were stained with PE-conjugated anti-hOX40L antibody. The expression of hOX40L on the surface of infected cells was evaluated by FACS analysis. FIG. 15A shows that the expression levels of GFP and hOX40L in infected BHK21 cells.

To test whether MVAΔC7L-hFlt3L-TK(−)-hOX40L can infect human monocyte-derived DCs (mo-DCs), adherent human peripheral blood mononuclear cells (PBMCs) were cultured in the presence of human GM-CSF and IL-4 for 5 days. On Day 6, cells were either treated with poly I:C at 10 μg/ml, or infected with Heat-iMVA, MVAΔC7L-TK(−), or MVAΔC7L-hFlt3L-TK(−)-hOX40L at MOI of 1. At 24 h post infection, cells were collected and stained with PE-conjugated anti-hOX40L antibody prior to FACS analyses. Compared with murine B16-F10 cells, human mo-DCs express hOX40L at the baseline. Poly I:C treatment leads to modest increase of hOX40L expression on the cell surface. Infection with Heat-iMVA reduces the expression of hOX40L. Both MVAΔC7L-hFlt3L and MVAΔC7L-TK(−)-hOX40L infection of mo-DCs resulted in GFP⁺ cells, around 50% and 75%, respectively. Only MVAΔC7L-TK(−)-hOX40L infection led to the increased expression of hOX40L on infected mo-DCs (FIG. 15B). These results indicate that MVAΔC7L-TK(−)-hOX40L can effectively infect human mo-DCs and express hOX40L on the surface of infected cells. This can be potentially important for interacting with T cells through OX40L-OX40 interaction, as well as promoting the survival and proliferation of antigen-specific and activated CD8⁺ and CD4⁺ T cells.

### Example 13: The Recombinant Viruses MVAΔC7L-hFlt3L-TK(−)-hOX40L Express hOX40L mRNA at High Levels in Infected Cells

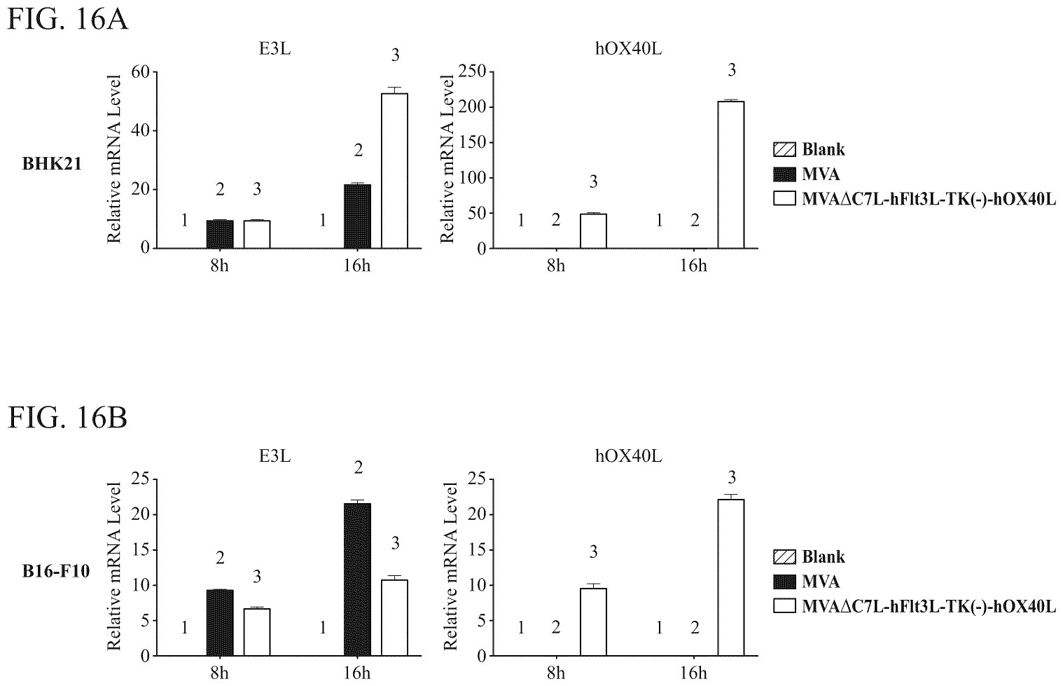

Quantitative RT-PCR analysis was used to assess the hOX40L mRNA expression level in infected BHK21 and B16-F10 melanoma cells. Briefly, BHK21 and B16-F10 melanoma cells were infected with either MVA or MVAΔC7L-hFlt3L or MVAΔC7L-TK(−)-hOX40L for 8 or 16 h. RNAs were extracted from the cells and quantitative RT-PCR analysis was performed to assess the expression of hOX40L mRNA. Vaccinia E3L mRNA levels were also assessed. Infection of BHK21 and B16-F10 melanoma cells results in the expression of both E3L and hOX40L at 8 and 16 h post infection. The mRNA levels of both E3L and hOX40L at 16 h were higher than those at 8 h post infection (FIGS. 16A and 16B). Overall, the expression of hOX40L was higher in MVAΔC7L-TK(−)-hOX40L-infected BHK21 cells compared with that in B16-F10 cells, which is consistent with the replication efficiency of this virus in BHK21 (permissive) and B16-F10 cells (non-permissive).

### Example 14: Cloning of Vaccinia Viral Early Genes into Expression Vectors

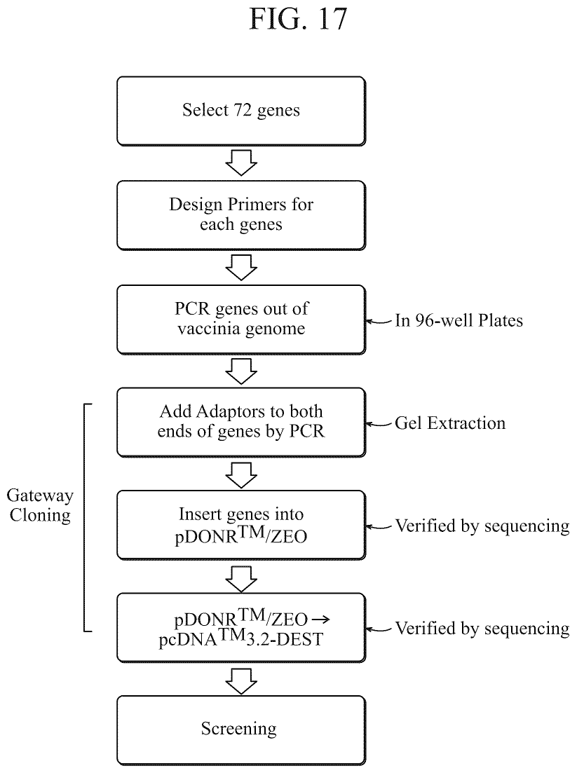

Vaccinia viral early genes were screened for their abilities to inhibit the cGAS/STING cytosolic DNA-sensing pathway. To do that, 72 vaccinia viral early genes were selected and their open reading frames were PCR-amplified and cloned into expression vector (pcDNA3.2-DEST) using Gateway Cloning technology (FIG. 17).

### Example 15: Screening Strategy for Identifying Viral Inhibitors of the cGAS/STING Pathway

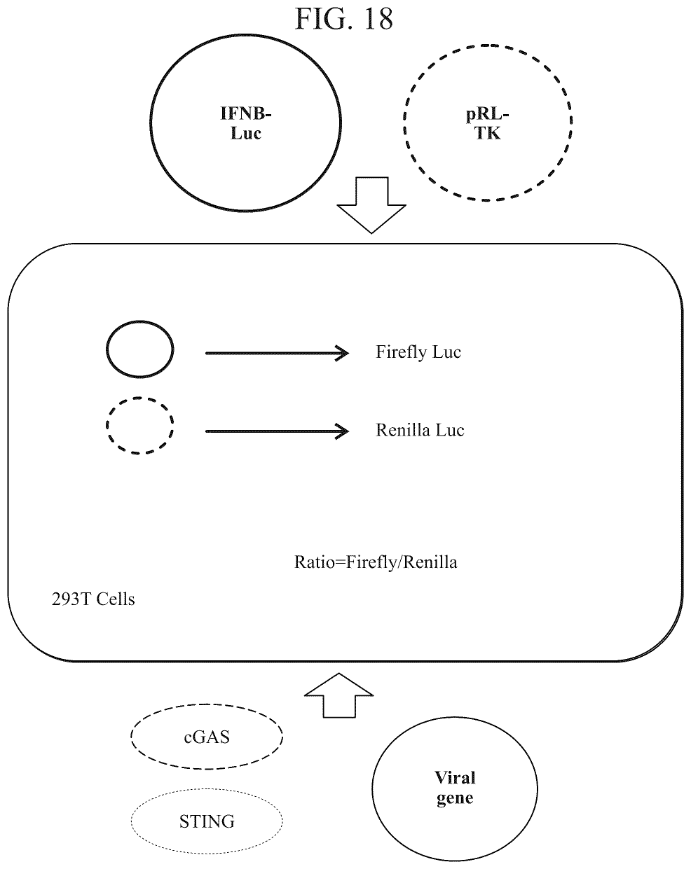

A dual-luciferase assay system was used to screen for potential vaccinia viral inhibitors of the cGAS/STING pathway in HEK293T-cells, a human embryonic kidney cell line transformed with SV40 large T antigen (FIG. 18). HEK293T-cells were transfected with plasmids expressing IFNB-firefly luciferase reporter, a control plasmid pRL-TK that expresses Renilla luciferase, murine cGAS, human STING, and individual vaccinia viral early gene as indicated. Murine cGAS (50 ng) and hSTING (10 ng) were used at suboptimal dosages for the purpose of identifying inhibitors. The transfected plasmids containing viral genes were used at 200 ng. IFNB-firefly luciferase reporter and control plasmid pRL-TK were used at 50 ng and 10 ng, respectively. Dual luciferase assays were performed at 24 h post transfection. The relative luciferase activity was expressed as arbitrary units by normalizing firefly luciferase activity to Renilla luciferase activity. Adenovirus E1A has been shown to inhibit this pathway through interacting with STING (Lau et al., Science (2015)) and was used as a positive control for this screening assay.

### Example 16: Identification of 8 Vaccinia Viral Early Genes that have Potential to Inhibit the cGAS/STING Pathway

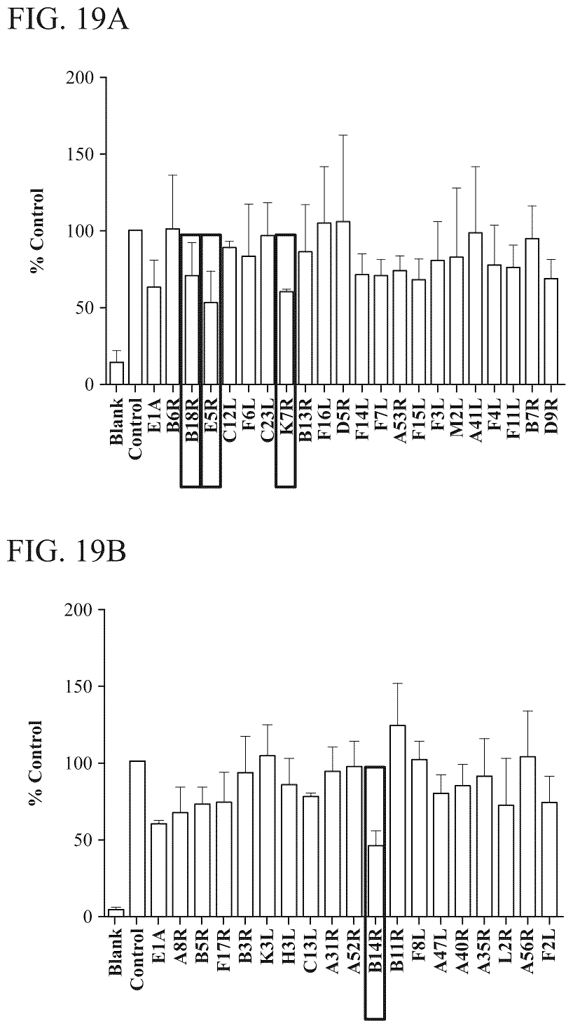

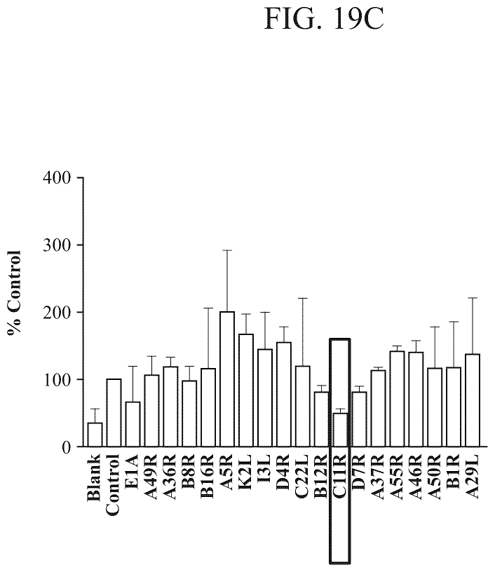

A dual-luciferase assay system described above was used to screen for vaccinia viral inhibitors of the cGAS/STING pathway. A total of eight vaccinia viral early genes (B18R (WR200), E5R, K7R, B14R, C11R, M1L, N2L, and WR199) were identified as potential inhibitors of this pathway. Data relating to five of these vaccinia viral early genes (B18R (WR200), E5R, K7R, B14R, and C11R) are shown in FIGS. 19A-19C. Those include some viral genes that have known inhibitory function of the type I IFN system, including B18R (WR200), which encodes a type I IFN binding protein (Alcami et al., (1995)). The roles of E5R, K7R, B14R, and C11R in the type I IFN pathway have not been elucidated previously, although K7R, B14R and C11R have been described as vaccinia virulence factors (Benfield et al., J. Gen. Virol. (2013), Chen et al., (2008); McCoy et al., (2010); Martin et al., (2012)). E5R has been reported to be a viral early protein associated with virosome (Murcia-Nicolas et al., (1999)). Its role in immune evasion has never been reported.

### Example 17: Confirmation that Overexpression of B18R, E5R, K7R, CUR, or B14R Down-Regulates IFNB Gene Expression Induced by the Co-Transfection of cGAS and hSTING in HEK293-T Cells

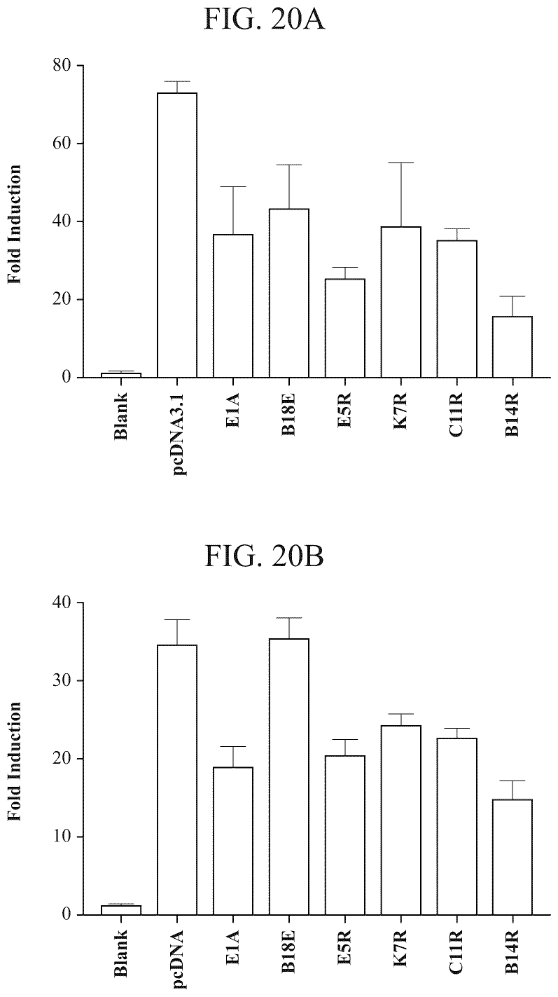

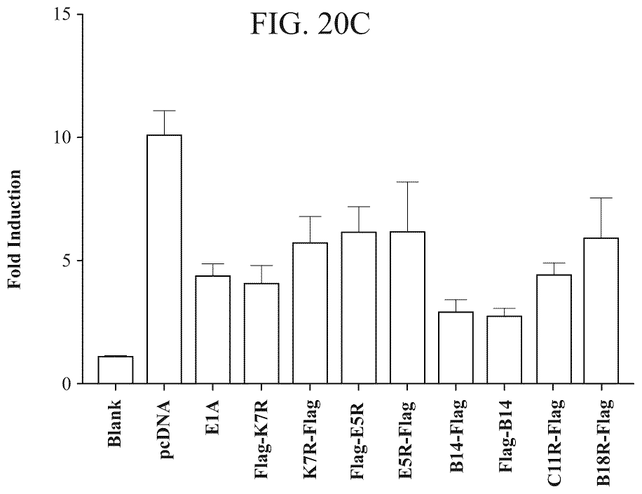

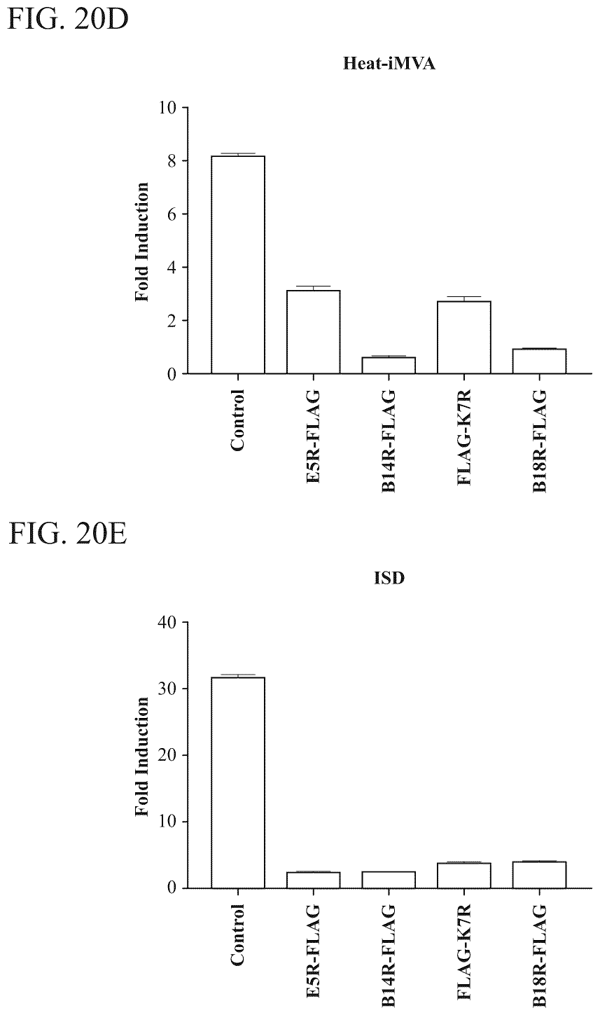

To confirm that B18R (WR200), E5R, K7R, C11R, and B14R play inhibitory roles in the cGAS/STING-induced IFNB gene expression, HEK293T-cells were co-transfected with plasmids expressing IFNB-firefly luciferase reporter, a control plasmid pRL-TK that expresses Renilla luciferase, murine cGAS (FIG. 20A), or human cGAS (FIG. 20B), human STING, individual vaccinia viral early gene as indicated, as well as E1A as a positive control. Overexpression of B18R (WR200), E5R, K7R, C11R, or B14R genes resulted in the reduction of IFNB gene expression induced by murine cGAS/human STING (FIG. 20A). Overexpression of E5R, K7R, C11R, or B14R genes resulted in the reduction of IFNB gene expression induced by human cGAS/human STING. However, overexpression of B18R (WR200) fails to inhibit IFNB gene expression induced by human cGAS/human STING (FIG. 20B). FIG. 20C shows that FLAG-tagged viral genes B18R (WR200), E5R, K7R, C11R or B14R were capable of inhibiting IFNB gene expression induced by murine cGAS/human STING.

### Example 18: Over-Expression of FLAG-Tagged E5R, B14R, K7R, and B18R Genes in a Stable Murine Macrophage Cell Line Inhibits IFNB Gene Expression Induced by Infection with Heat-Inactivated MVA or Transfection with Immune-Stimulating DNA

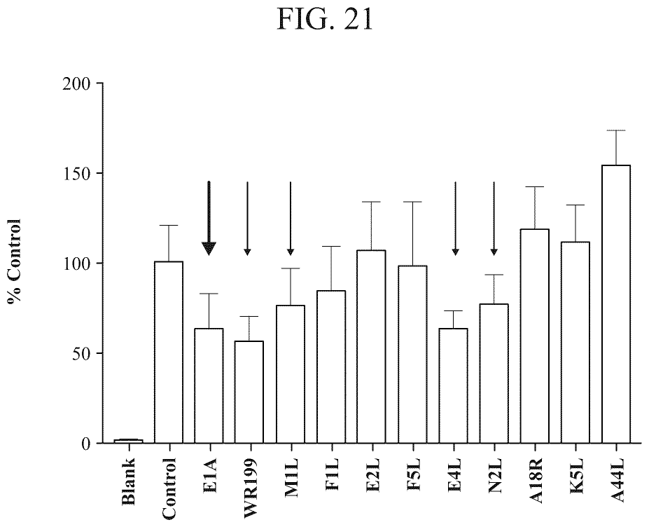

A stable cell line, RAW264.7, that overexpresses either E5R-FLAG, B14R-FLAG, FLAG-K7R, or B18R-FLAG genes was generated. Briefly, RAW264.7 were transduced with retrovirus containing the expression construct of vaccinia E5R-FLAG, B14R-FLAG, FLAG-K7R, or B18R-FLAG genes under CMV promoter and puromycin selection marker. Empty vector with drug selection marker was also used to generate a control cell line. Drug resistant cells were obtained and used for the following experiments. Cells were either infected with Heat-iMVA or transfected with immune-stimulating DNA (ISD) (10 μg/ml). At 12 h post treatment, cells were collected. RNAs were generated and quantitative RT-PCR was performed to evaluate the expression of IFNB gene. Infection with Heat-iMVA or treatment with ISD in the control cell line resulted in 8- and 32-fold induction of IFNB gene, respectively. The induction of IFNB was markedly reduced in cells over-expressing E5, B14, K7, or B18 (FIG. 21). These results indicate that vaccinia E5R, B14R, K7R, and B18R inhibit the cytosolic DNA-sensing pathway and blocks IFNB gene induction.

### Example 19: Generation of Recombinant MVAΔE5R, MVAΔK7R, or MVAΔB14R Viruses

To further establish the role of E5R, K7R, and B14R in immune modulation, the generation of MVAΔE5R, MVAΔK7R, and/or MVAΔB14R viruses will be established. pE5RGFP vector, pK7RGFP vector, or pB14RGFP vector will be constructed to insert a specific gene of interest (SG) into the E5R, K7R, or B14R loci of MVA. In this case, GFP under the control of the vaccinia P7.5 promoter will be used as a selection marker. BHK21 cells will be infected with MVA virus expressing LacZ at a MOI of 0.05 for 1 h, and then will be transfected with the plasmid DNA described above. The infected cells will be collected at 48 h. Recombinant viruses will be identified by their green fluorescence with the insertion of GFP into the E5R, K7R, or B14R loci. The positive clones will be plaque purified 4-5 times. PCR analysis will then be performed to confirm that the recombinant viruses MVAΔE5R, MVAΔK7R, or MVAΔB14R have lost the E5R, K7R, or B14R, respectively.

### Example 20: MVAΔE5R, MVAΔK7R, or MVAΔB14R Infection of cDCs Induces Higher Levels of Type I IFN Gene Expression than MVA

MVA infection of conventional dendritic cells (cDCs) has been shown to induce type I IFN via a cGAS/STING/IRF3-dependent mechanism. To test whether E5R, K7R, or B14R plays an inhibitory role in the induction of cytosolic DNA-sensing pathway, the innate immune responses of bone marrow-derived DCs (BMDCs) to MVAΔE5R, MVAΔK7R, and/or MVAΔB14R vs. MVA will be analyzed. BMDCs will be infected with either MVAΔE5R, MVAΔK7R, MVAΔB14R, or MVA at a MOI of 10. Cells will be collected at 3 h and 6 h post infection. The type I IFN gene expression levels will be determined by quantitative PCR analyses. It is anticipated that MVAΔE5R, MVAΔK7R, or MVAΔB14R infection will induce significantly higher levels of type I IFN gene expression than MVA in cDCs at 3 h and 6 h post infection. To test whether MVAΔE5R, MVAΔK7R, or MVAΔB14R will induce higher levels of type I IFN gene activation in human immune cells, the widely used differentiated THP-1 cells will be employed. THP-1 cells will be infected with either MVAΔE5R, MVAΔK7R, MVAΔB14R, or MVA at a MOI of 10, and then will be collected at 3 h and 6 h post infection. It is anticipated that MVAΔE5R, MVAΔK7R, or MVAΔB14R infection will induce higher levels of type I IFN gene expression than MVA in THP-1 cells. These results will indicate that E5R, K7R, and/or B14R are inhibitors that antagonize the cytosolic DNA-sensing pathway. Accordingly, these results will show that MVAΔE5R, MVAΔK7R, and/or MVAΔB14R may be useful in methods of inducing the innate immune response.

### Example 21: Generation of a Cytokine Producing Recombinant Modified Vaccinia Ankara (MVA) Virus Using MVAΔC7L-hFlt3L-TK(−)-OX40L Recombinant Virus as a Backbone to Include E5R, K7R, and/or B14R Deletions, and to Express IL-2, IL-12, IL-18, IL-15, and/or IL-21 and its Use in Methods for Treating Solid Tumors Alone or in Combination with Immune Checkpoint Blockade Agents

This example describes the generation of a recombinant MVAΔC7L-hFlt3L-TK(−)-OX40L virus expressing transgenes IL-2, IL-12, IL-18, IL-15, and/or IL-21 from within either the E5R gene locus, the K7R gene locus, and/or the B14R gene locus and its use in methods for treating solid tumors alone or in combination with immune checkpoint blockade agents.

The recombinant virus will be engineered according to the homologous recombination methods described in the preceding examples. For example, expression cassettes will be designed to express IL-2, IL-12, IL-18, IL-15, and/or IL-21 using the vaccinia viral synthetic early and late promoter (PsE/L) and GFP or the E. coli xanthine-guanine phosphoribosyl transferase gene (gpt) under the control of the vaccinia P7.5 promoter used as a selection marker. The expression cassettes will be flanked by partial sequences of the gene into which the cassettes will be inserted via homologous recombination (e.g., the E5R gene, the K7R gene, or the B14R gene). BHK21 cells will be infected with recombinant vaccinia virus at a multiplicity of infection (MOI) of 0.05 for 1 h, and then will be transfected with the plasmid DNAs described above. The infected cells will be collected at 48 h. Recombinant viruses are selected through further culturing in gpt selection medium including MPA, xanthine and hypoxanthine, and plaque purified. PCR analysis will be performed to verify that the MVAΔC7L-hFlt3L-TK(−)-OX40L virus lacks the E5R gene, the K7R gene, and/or the B14R gene, but with IL-2, IL-12, IL-18, IL-15, and/or IL-21 insertion. The expression of the transgenes on murine B16-F10 cells and human SK-MEL-28 cells infected with the recombinant virus will be determined by FACS analysis using the appropriate antibody. It is anticipated that the majority of both murine B16-F10 and SK-MEL28 cells will express the transgene.

A bilateral tumor implantation model will be used to assess the anti-tumor efficacy of the recombinant viruses. Briefly, B16-F10 melanoma cells will be implanted intradermally into the shaved skin on the right (5×10⁵ cells) and left (1×10⁵ cells) flanks of a C57BL/6J mouse. After 7 to 8 days post implantation, the mice will be injected twice per week with: (i) PBS; (ii) intraperitoneal (IP) MVAΔC7L-hFlt3L-TK(−)-OX40L virus expressing transgenes IL-2, IL-12, IL-18, IL-15, and/or IL-21 from within either the E5R gene locus, the K7R gene locus, and/or the B14R gene locus; (iii) intratumoral (IT) MVAΔC7L-hFlt3L-TK(−)-OX40L virus expressing transgenes IL-2, IL-12, IL-18, IL-15, and/or IL-21 from within either the E5R gene locus, the K7R gene locus, and/or the B14R gene locus plus; (iv) intraperitoneal (IP) and intratumoral (IT) MVAΔC7L-hFlt3L-TK(−)-OX40L virus expressing transgenes IL-2, IL-12, IL-18, IL-15, and/or IL-21 from within either the E5R gene locus, the K7R gene locus, or the B14R gene locus; or (v) intratumoral (IT) MVAΔC7L-hFlt3L-TK(−)-OX40L virus expressing transgenes IL-2, IL-12, IL-18, IL-15, and/or IL-21 from within either the E5R gene locus, the K7R gene locus, and/or the B14R gene locus plus intraperitoneal (IP) immune checkpoint blockade agent (e.g., anti-PD-L1 antibody, anti-PD-1 antibody, anti-CTLA-4 antibody) when the mice are under anesthesia. The mice will be monitored for survival and the tumor sizes will be measured twice a week.

The results of this example will demonstrate the anti-tumor efficacy of MVAΔC7L-hFlt3L-TK(−)-OX40L virus expressing transgenes IL-2, IL-12, IL-18, IL-15, and/or IL-21 from within either the E5R gene locus, the K7R gene locus, and/or the B14R gene locus. It is anticipated that the IP and/or IT administration of MVAΔC7L-hFlt3L-TK(−)-OX40L virus expressing transgenes IL-2, IL-12, IL-18, IL-15, and/or IL-21 from within either the E5R gene locus, the K7R gene locus, and/or the B14R gene locus to mice with solid tumors will induce an anti-tumoral response, reduce tumor size, and/or increase survival. It is also anticipated that the combined administration of MVAΔC7L-hFlt3L-TK(−)-OX40L virus expressing transgenes IL-2, IL-12, IL-18, IL-15, and/or IL-21 from within either the E5R gene locus, the K7R gene locus, and/or the B14R gene locus and immune checkpoint blockade agent (e.g., anti-PD-L1 antibody, anti-PD-1 antibody, anti-CTLA-4 antibody) will induce an anti-tumoral response, reduce tumor size, and/or increase survival. It is further anticipated that the combined administration of MVAΔC7L-hFlt3L-TK(−)-OX40L virus expressing transgenes IL-2, IL-12, IL-18, IL-15, and/or IL-21 from within either the E5R gene locus, the K7R gene locus, and/or the B14R gene locus and immune checkpoint blockade agent (e.g., anti-PD-L1 antibody, anti-PD-1 antibody, anti-CTLA-4 antibody) will produce synergistic effects in this regard as compared to the administration of MVAΔC7L-hFlt3L-TK(−)-OX40L virus expressing transgenes IL-2, IL-12, IL-18, IL-15, and/or IL-21 from within either the E5R gene locus, the K7R gene locus, and/or the B14R gene locus or immune checkpoint blockade therapy alone.

Accordingly, this example demonstrates that compositions of the present technology comprising recombinant MVAΔC7L-hFlt3L-TK(−)-OX40L viruses expressing transgenes IL-2, IL-12, IL-18, IL-15, and/or IL-21 from within either the E5R gene locus, the K7R gene locus, and/or the B14R gene locus alone or in combination with immune checkpoint blockade agents are useful in methods for treating solid tumors.

### Example 22: Generation of Recombinant Vaccinia Virus with Deletion of E5R, K7R, or B14R (VACVΔE5R, VACVΔK7R, or VACVΔB14R)

pC7LGFP vector will be used to insert GFP under the control of the vaccinia P7.5 promoter into the E5R, K7R, or B14R loci of vaccinia virus (VACV). The expression cassette will be flanked by partial sequence of E5R, K7R, or B14R flank regions on each side. BSC40 cells will be infected with WT vaccinia virus expressing at a MOI of 0.05 for 1 h, and then will be transfected with the plasmid DNA described above. The infected cells will be collected at 48 h. Recombinant viruses will be identified by their green fluorescence with the insertion of GFP into the E5R, K7R, or B14R loci. The positive clones will then be plaque purified 4-5 times on BSC40 cells. PCR analysis will be performed to confirm that recombinant viruses VACVΔE5R, VACVΔK7R, or VACVΔB14R have lost the E5R, K7R, or B14R, respectively.

To determine if one of E5R, K7R, or B14R is a virulence factor and if VACVΔE5R, VACVΔK7R, or VACVΔB14R is highly attenuated compared to WT VACV, a murine intranasal infection model will be employed. Weight loss in C57BL/6J mice after intranasal infection with various doses of WT VACV will be compared to that observed in C57BL/6J after infection with VACVΔE5R, VACVΔK7R, or VACVΔB14R.

### Example 23: Generation of a Recombinant Vaccinia Virus with a TK Deletion, Disruption of the E3L Gene, C7 Deletion, and Expressing hFlt3L, Anti-CTLA-4, and OX40L (VACVΔE3L83N-hFlt3L-Anti-CTLA-4-ΔC7L-OX40L) and its Use in Methods for Treating Solid Tumors Alone or in Combination with Immune Checkpoint Blockade Agents

This example describes the generation of a recombinant vaccinia E3LΔ83N virus comprising a TK deletion, a C7 deletion, and expressing an antibody that specifically targets cytotoxic T lymphocyte antigen 4 (anti-CTLA-4), hFlt3L, and OX40L and its use in methods for treating solid tumors alone or in combination with immune checkpoint blockade agents.

The virus is generated using plasmids containing expression cassettes designed to express one or more specific genes of interest (SG) (e.g., anti-CTLA-4, OX40L, hFtl3L). The expression cassettes are designed to express anti-CTLA-4, OX40L, and/or hFtl3L using the vaccinia viral synthetic early and late promoter (PsE/L) and GFP or the E. coli xanthine-guanine phosphoribosyl transferase gene (gpt) under the control of the vaccinia P7.5 promoter used as a selection marker. For example, an expression cassette is flanked by a partial sequence of C8L and C6R on the left and right side of C7L gene for insertion of a specific gene(s) of interest into the C7 locus via homologous recombination. An expression cassette may be flanked by the thymidine kinase (TK) gene on either side (TK-L, TK-R) for insertion of a specific gene(s) of interest into the TK locus via homologous recombination. BHK21 cells are infected with recombinant vaccinia virus at a multiplicity of infection (MOI) of 0.05 for 1 h, and then transfected with the plasmid DNAs described above. The infected cells are collected at 48 h. Recombinant viruses are selected through further culturing in selection medium including MPA, xanthine and hypoxanthine, and plaque purified. PCR analysis is performed to verify that VACVΔE3L83N-hFlt3L-anti-CTLA-4-ΔC7L-OX40L lacks C7L gene and part of the TK gene, but with hFlt3L, anti-CTLA-4, and OX40L insertion. The expression of OX40L on murine B16-F10 cells and human SK-MEL-28 cells infected with VACVΔE3L83N-hFlt3L-anti-CTLA-4-ΔC7L-OX40L virus is determined by FACS analysis using anti-OX40L antibody. It is anticipated that the majority of both murine B16-F10 and SK-MEL28 cells will express OX40L.

A bilateral tumor implantation model is used to assess the anti-tumor efficacy of the recombinant viruses. Briefly, B16-F10 melanoma cells are implanted intradermally into the shaved skin on the right (5×10⁵ cells) and left (1×10⁵ cells) flanks of a C57BL/6J mouse. After 7 to 8 days post implantation, the mice are injected twice per week with: (i) PBS; (ii) intraperitoneal (IP) VACVΔE3L83N-hFlt3L-anti-CTLA-4-ΔC7L-OX40L; (iii) intratumoral (IT) VACVΔE3L83N-hFlt3L-anti-CTLA-4-ΔC7L-OX40L; (iv) intraperitoneal (IP) and intratumoral (IT) VACVΔE3L83N-hFlt3L-anti-CTLA-4-ΔC7L-OX40L; or (v) intratumoral (IT) VACVΔE3L83N-hFlt3L-anti-CTLA-4-ΔC7L-OX40L plus intraperitoneal (IP) immune checkpoint blockade agent (e.g., anti-PD-L1 antibody, anti-PD-1 antibody, anti-CTLA-4 antibody) when the mice are under anesthesia. The mice are monitored for survival and the tumor sizes are measured twice a week.

The results of this example will demonstrate the anti-tumor efficacy of VACVΔE3L83N-hFlt3L-anti-CTLA-4-ΔC7L-OX40L. It is anticipated that the IP and/or IT administration of VACVΔE3L83N-hFlt3L-anti-CTLA-4-ΔC7L-OX40L to mice with solid tumors will induce an anti-tumoral response, reduce tumor size, and/or increase survival. It is also anticipated that the combined administration of VACVΔE3L83N-hFlt3L-anti-CTLA-4-ΔC7L-OX40L and immune checkpoint blockade agent (e.g., anti-PD-L1 antibody, anti-PD-1 antibody, anti-CTLA-4 antibody) will induce an anti-tumoral response, reduce tumor size, and/or increase survival. It is further anticipated that the combined administration of VACVΔE3L83N-hFlt3L-anti-CTLA-4-ΔC7L-OX40L and immune checkpoint blockade agent (e.g., anti-PD-L1 antibody, anti-PD-1 antibody, anti-CTLA-4 antibody) will produce synergistic effects in this regard as compared to the administration of VACVΔE3L83N-hFlt3L-anti-CTLA-4-ΔC7L-OX40L or immune checkpoint blockade therapy alone.

Accordingly, this example demonstrates that compositions of the present technology comprising recombinant VACVΔE3L83N-hFlt3L-anti-CTLA-4-ΔC7L-OX40L virus alone or in combination with immune checkpoint blockade agents are useful in methods for treating solid tumors.

### Example 24: Generation of Cytokine Producing Recombinant Vaccinia Virus Using VACVΔE3L83N-hFlt3L-Anti CTLA-4-ΔC7L-OX40L Recombinant Virus as a Backbone to Insert IL-2, IL-12, IL-18, IL-15, and/or IL-21 into the E5R, K7R, and/or B14R Locus and its Use in Methods for Treating Solid Tumors Alone or in Combination with Immune Checkpoint Blockade Agents

This example describes the generation of a recombinant VACVΔE3L83N-hFlt3L-anti-CTLA-4-ΔC7L-OX40L virus expressing transgenes IL-2, IL-12, IL-18, IL-15, and/or IL-21 from within either the E5R gene locus, the K7R gene locus, and/or the B14R gene locus and its use in methods for treating solid tumors alone or in combination with immune checkpoint blockade agents.

The recombinant virus will be engineered according to the homologous recombination methods described in the preceding examples. For example, expression cassettes are designed to express IL-2, IL-12, IL-18, IL-15, and/or IL-21 using the vaccinia viral synthetic early and late promoter (PsE/L) and GFP or the E. coli xanthine-guanine phosphoribosyl transferase gene (gpt) under the control of the vaccinia P7.5 promoter used as a selection marker. The expression cassettes are flanked by partial sequences of the gene into which the cassettes will be inserted via homologous recombination (e.g., the E5R gene, the K7R gene, or the B14R gene). BHK21 cells are infected with recombinant vaccinia virus at a multiplicity of infection (MOI) of 0.05 for 1 h, and then transfected with the plasmid DNAs described above. The infected cells are collected at 48 h. Recombinant viruses are selected through further culturing in gpt selection medium including MPA, xanthine and hypoxanthine, and plaque purified. PCR analysis is performed to verify that the VACVΔE3L83N-hFlt3L-anti-CTLA-4-ΔC7L-OX40L virus lacks the E5R gene, the K7R gene, and/or the B14R gene, but with IL-2, IL-12, IL-18, IL-15, and/or IL-21 insertion. The expression of the transgenes on murine B16-F10 cells and human SK-MEL-28 cells infected with the recombinant virus is determined by FACS analysis using the appropriate antibody. It is anticipated that the majority of both murine B16-F10 and SK-MEL28 cells will express the transgene(s).

A bilateral tumor implantation model is used to assess the anti-tumor efficacy of the recombinant viruses. Briefly, B16-F10 melanoma cells are implanted intradermally into the shaved skin on the right (5×10⁵ cells) and left (1×10⁵ cells) flanks of a C57BL/6J mouse. After 7 to 8 days post implantation, the mice are injected twice per week with: (i) PBS; (ii) intraperitoneal (IP) VACVΔE3L83N-hFlt3L-anti-CTLA-4-ΔC7L-OX40L virus expressing transgenes IL-2, IL-12, IL-18, IL-15, and/or IL-21 from within the E5R gene locus, the K7R gene locus, and/or the B14R gene locus; (iii) intratumoral (IT) VACVΔE3L83N-hFlt3L-anti-CTLA-4-ΔC7L-OX40L virus expressing transgenes IL-2, IL-12, IL-18, IL-15, and/or IL-21 from within the E5R gene locus, the K7R gene locus, and/or the B14R gene locus plus; (iv) intraperitoneal (IP) and intratumoral (IT) VACVΔE3L83N-hFlt3L-anti-CTLA-4-ΔC7L-OX40L virus expressing transgenes IL-2, IL-12, IL-18, IL-15, and/or IL-21 from within the E5R gene locus, the K7R gene locus, and/or the B14R gene locus; or (v) intratumoral (IT) VACVΔE3L83N-hFlt3L-anti-CTLA-4-ΔC7L-OX40L virus expressing transgenes IL-2, IL-12, IL-18, IL-15, and/or IL-21 from within the E5R gene locus, the K7R gene locus, and/or the B14R gene locus plus intraperitoneal (IP) immune checkpoint blockade agent (e.g., anti-PD-L1 antibody, anti-PD-1 antibody, anti-CTLA-4 antibody) when the mice are under anesthesia. The mice are monitored for survival and the tumor sizes are measured twice a week.

The results of this example will demonstrate the anti-tumor efficacy of VACVΔE3L83N-hFlt3L-anti-CTLA-4-ΔC7L-OX40L virus expressing transgenes IL-2, IL-12, IL-18, IL-15, and/or IL-21 from within the E5R gene locus, the K7R gene locus, and/or the B14R gene locus. It is anticipated that the IP and/or IT administration of VACVΔE3L83N-hFlt3L-anti-CTLA-4-ΔC7L-OX40L virus expressing transgenes IL-2, IL-12, IL-18, IL-15, and/or IL-21 from within the E5R gene locus, the K7R gene locus, and/or the B14R gene locus to mice with solid tumors will induce an anti-tumoral response, reduce tumor size, and/or increase survival. It is also anticipated that the combined administration of VACVΔE3L83N-hFlt3L-anti-CTLA-4-ΔC7L-OX40L virus expressing transgenes IL-2, IL-12, IL-18, IL-15, and/or IL-21 from within the E5R gene locus, the K7R gene locus, and/or the B14R gene locus and immune checkpoint blockade agent (e.g., anti-PD-L1 antibody, anti-PD-1 antibody, anti-CTLA-4 antibody) will induce an anti-tumoral response, reduce tumor size, and/or increase survival. It is further anticipated that the combined administration of VACVΔE3L83N-hFlt3L-anti-CTLA-4-ΔC7L-OX40L virus expressing transgenes IL-2, IL-12, IL-18, IL-15, and/or IL-21 from within the E5R gene locus, the K7R gene locus, and/or the B14R gene locus and immune checkpoint blockade agent (e.g., anti-PD-L1 antibody, anti-PD-1 antibody, anti-CTLA-4 antibody) will produce synergistic effects in this regard as compared to the administration of VACVΔE3L83N-hFlt3L-anti-CTLA-4-ΔC7L-OX40L virus expressing transgenes IL-2, IL-12, IL-18, IL-15, and/or IL-21 from within the E5R gene locus, the K7R gene locus, and/or the B14R gene locus or immune checkpoint blockade therapy alone.

Accordingly, this example demonstrates that compositions of the present technology comprising recombinant VACVΔE3L83N-hFlt3L-anti-CTLA-4-ΔC7L-OX40L viruses expressing transgenes IL-2, IL-12, IL-18, IL-15, and/or IL-21 from within the E5R gene locus, the K7R gene locus, and/or the B14R gene locus alone or in combination with immune checkpoint blockade agents are useful in methods for treating solid tumors.

### Example 25: Generation of a Recombinant C7L Mutant Vaccinia Virus with a TK Deletion and Expressing hFlt3L and OX40L (VACVΔC7L-TK(−)-hFlt3L-OX40L) and its Use in Methods for Treating Solid Tumors Alone or in Combination with Immune Checkpoint Blockade Agents

This example describes the generation of a recombinant VACVΔC7L-TK(−)-hFlt3L-OX40L virus and its use in methods for treating solid tumors alone or in combination with immune checkpoint blockade agents.

The virus is generated using plasmids containing expression cassettes designed to express one or more specific genes of interest (SG) (e.g., OX40L, hFtl3L). The expression cassettes are designed to express OX40L and/or hFtl3L using the vaccinia viral synthetic early and late promoter (PsE/L) and GFP or the E. coli xanthine-guanine phosphoribosyl transferase gene (gpt) under the control of the vaccinia P7.5 promoter used as a selection marker. For example, an expression cassette is flanked by a partial sequence of C8L and C6R on the left and right side of C7L gene for insertion of a specific gene(s) of interest (e.g., hFlt3L) into the C7 locus via homologous recombination. An expression cassette may be flanked by the thymidine kinase (TK) gene on either side (TK-L, TK-R) for insertion of a specific gene(s) of interest (e.g., OX40L) into the TK locus via homologous recombination. BHK21 cells are infected with recombinant vaccinia virus at a multiplicity of infection (MOI) of 0.05 for 1 h, and then transfected with the plasmid DNAs described above. The infected cells are collected at 48 h. Recombinant viruses are selected through further culturing in selection medium including MPA, xanthine and hypoxanthine, and plaque purified. PCR analysis is performed to verify that VACVΔC7L-TK(−)-hFlt3L-OX40L lacks C7L gene and part of the TK gene, but with hFlt3L, and OX40L insertion. The expression of OX40L and hFlt3L on murine B16-F10 cells and human SK-MEL-28 cells infected with VACVΔC7L-TK(−)-hFlt3L-OX40L virus is determined by FACS analysis using anti-OX40L and anti-hFlt3L antibody. It is anticipated that both murine B16-F10 and SK-MEL28 cells will express OX40L and hFlt3L.

A bilateral tumor implantation model is used to assess the anti-tumor efficacy of the recombinant viruses. Briefly, B16-F10 melanoma cells are implanted intradermally into the shaved skin on the right (5×10⁵ cells) and left (1×10⁵ cells) flanks of a C57BL/6J mouse. After 7 to 8 days post implantation, the mice are injected twice per week with: (i) PBS; (ii) intraperitoneal (IP) VACVΔC7L-TK(−)-hFlt3L-OX40L; (iii) intratumoral (IT) VACVΔC7L-TK(−)-hFlt3L-OX40L virus; (iv) intraperitoneal (IP) and intratumoral (IT) VACVΔC7L-TK(−)-hFlt3L-OX40L virus; or (v) intratumoral (IT) VACVΔC7L-TK(−)-hFlt3L-OX40L virus plus intraperitoneal (IP) immune checkpoint blockade agent (e.g., anti-PD-L1 antibody, anti-PD-1 antibody, anti-CTLA-4 antibody) when the mice are under anesthesia. The mice are monitored for survival and the tumor sizes are measured twice a week.

The results of this example will demonstrate the anti-tumor efficacy of VACVΔC7L-TK(−)-hFlt3L-OX40L virus. It is anticipated that the IP and/or IT administration of VACVΔC7L-TK(−)-hFlt3L-OX40L virus to mice with solid tumors will induce an anti-tumoral response, reduce tumor size, and/or increase survival. It is also anticipated that the combined administration of VACVΔC7L-TK(−)-hFlt3L-OX40L virus and immune checkpoint blockade agent (e.g., anti-PD-L1 antibody, anti-PD-1 antibody, anti-CTLA-4 antibody) will induce an anti-tumoral response, reduce tumor size, and/or increase survival. It is further anticipated that the combined administration of VACVΔC7L-TK(−)-hFlt3L-OX40L virus and immune checkpoint blockade agent (e.g., anti-PD-L1 antibody, anti-PD-1 antibody, anti-CTLA-4 antibody) will produce synergistic effects in this regard as compared to the administration of VACVΔC7L-TK(−)-hFlt3L-OX40L virus or immune checkpoint blockade therapy alone.

Accordingly, this example demonstrates that compositions of the present technology comprising recombinant VACVΔC7L-TK(−)-hFlt3L-OX40L viruses alone or in combination with immune checkpoint blockade agents are useful in methods for treating solid tumors.

### Example 26: Generation of a Recombinant C7L Mutant Vaccinia Virus with a TK Deletion and Expressing Anti-CTLA-4, hFlt3L, OX40L, and hIL-12 (VACVΔC7L-Anti-CTLA-4-TK(−)-hFlt3L-OX40L-hIL-12) and its Use in Methods for Treating Solid Tumors Alone or in Combination with Immune Checkpoint Blockade Agents

This example describes the generation of a recombinant VACVΔC7L-TK(−)-anti-CTLA-4-TK(−)-hFlt3L-OX40L-hIL-12 virus and its use in methods for treating solid tumors alone or in combination with immune checkpoint blockade agents.

The recombinant virus will be engineered according to the homologous recombination methods described in the preceding examples. For example, expression cassettes are designed to express anti-CTLA-4, hFlt3L, OX40L, and/or hIL-12 using the vaccinia viral synthetic early and late promoter (PsE/1) and GFP or the E. coli xanthine-guanine phosphoribosyl transferase gene (gpt) under the control of the vaccinia P7.5 promoter used as a selection marker. The expression cassettes are flanked by partial sequences of the gene into which the cassettes will be inserted via homologous recombination (e.g., the C7 gene, the TK gene, or any other suitable vaccinia viral gene). BHK21 cells are infected with recombinant vaccinia virus at a multiplicity of infection (MOI) of 0.05 for 1 h, and then transfected with the plasmid DNAs described above. The infected cells are collected at 48 h. Recombinant viruses are selected through further culturing in selection medium including MPA, xanthine and hypoxanthine, and plaque purified. PCR analysis is performed to verify that VACVΔC7L-TK(−)-anti-CTLA-4-TK(−)-hFlt3L-OX40L-hIL-12 lacks C7L gene and part of the TK gene, but with transgenes anti-CTLA-4, hFlt3L, OX40L, and hIL-12 insertion. The expression of transgenes in murine B16-F10 cells and human SK-MEL-28 cells infected with VACVΔC7L-TK(−)-anti-CTLA-4-TK(−)-hFlt3L-OX40L-hIL-12 virus is determined by FACS analysis using the appropriate antibodies. It is anticipated that both murine B16-F10 and SK-MEL28 cells will express the transgenes.

A bilateral tumor implantation model is used to assess the anti-tumor efficacy of the recombinant viruses. Briefly, B16-F10 melanoma cells are implanted intradermally into the shaved skin on the right (5×10⁵ cells) and left (1×10⁵ cells) flanks of a C57BL/6J mouse. After 7 to 8 days post implantation, the mice are injected twice per week with: (i) PBS; (ii) intraperitoneal (IP) VACVΔC7L-TK(−)-anti-CTLA-4-TK(−)-hFlt3L-OX40L-hIL-12; (iii) intratumoral (IT) VACVΔC7L-TK(−)-anti-CTLA-4-TK(−)-hFlt3L-OX40L-hIL-12virus; (iv) intraperitoneal (IP) and intratumoral (IT) VACVΔC7L-TK(−)-anti-CTLA-4-TK(−)-hFlt3L-OX40L-hIL-12virus; or (v) intratumoral (IT) VACVΔC7L-TK(−)-anti-CTLA-4-TK(−)-hFlt3L-OX40L-hIL-12 virus plus intraperitoneal (IP) immune checkpoint blockade agent (e.g., anti-PD-L1 antibody, anti-PD-1 antibody) when the mice are under anesthesia. The mice are monitored for survival and the tumor sizes are measured twice a week.

The results of this example will demonstrate the anti-tumor efficacy of VACVΔC7L-TK(−)-anti-CTLA-4-TK(−)-hFlt3L-OX40L-hIL-12 virus. It is anticipated that the IP and/or IT administration of VACVΔC7L-TK(−)-anti-CTLA-4-TK(−)-hFlt3L-OX40L-hIL-12 virus to mice with solid tumors will induce an anti-tumoral response, reduce tumor size, and/or increase survival. It is also anticipated that the combined administration of VACVΔC7L-TK(−)-anti-CTLA-4-TK(−)-hFlt3L-OX40L-hIL-12 virus and immune checkpoint blockade agent (e.g., anti-PD-L1 antibody, anti-PD-1 antibody) will induce an anti-tumoral response, reduce tumor size, and/or increase survival. It is further anticipated that the combined administration of VACVΔC7L-TK(−)-anti-CTLA-4-TK(−)-hFlt3L-OX40L-hIL-12 virus and immune checkpoint blockade agent (e.g., anti-PD-L1 antibody, anti-PD-1 antibody) will produce synergistic effects in this regard as compared to the administration of VACVΔC7L-TK(−)-anti-CTLA-4-TK(−)-hFlt3L-OX40L-hIL-12 virus or immune checkpoint blockade therapy alone.

Accordingly, this example demonstrates that compositions of the present technology comprising recombinant VACVΔC7L-TK(−)-anti-CTLA-4-TK(−)-hFlt3L-OX40L-hIL-12 viruses alone or in combination with immune checkpoint blockade agents are useful in methods for treating solid tumors.

### Example 27: Co-Administration of MVAΔC7L-hFlt3L-TK(−)-mOX40L with Model Antigen, Chicken Ovalbumin (OVA), Enhances the Generation of OVA-Specific CD8⁺ and CD4⁺ T-Cells in the Spleen and Draining Lymph Nodes (dLNs), and Serum Anti-OVA IgG Antibodies in Immunized Mice

This example will demonstrate that MVAΔC7L-hFlt3L-TK(−)-mOX40L can act as a vaccine adjuvant to enhance antigen presentation by dendritic cells (DCs). Mice are immunized subcutaneously (SC) with OVA (10 μg) with or without MVAΔC7L-hFlt3L-TK(−)-mOX40L (1×10⁷ pfu) twice, 2 weeks apart. Mice are euthanized 1 week after the second vaccination, with spleens, draining lymph nodes (dLNs), and blood subsequently collected for OVA-specific T-cell and antibody assessment. To determine anti-OVA CD8⁺ T-cell responses, splenocytes (500,000 cells) are incubated with OVA 257-264 (SIINFEKL) peptide (SEQ ID NO: 15), which is a MHC class I (Kb)-restricted peptide epitope of OVA, for 12 h before they were stained for anti-CD8 and anti-IFN-γ antibodies. To test anti-OVA CD4⁺ T-cell responses, splenocytes (500,000 cells) are incubated with OVA 323-339 (ISQAVHAAHAEINEAGR) peptide (SEQ ID NO: 16), which is a MHC class II I-A^{d}-restricted peptide epitope of OVA, for 12 h before they were stained for anti-CD4 and anti-IFN-γ antibodies. Co-administration of MVAΔC7L-hFlt3L-TK(−)-mOX40L with OVA SC is anticipated to result in the increase of anti-OVA IFN-γ⁺CD8⁺ T-cells and anti-OVA IFN-γ⁺CD4⁺ T-cells in the spleens compared with OVA alone.

A similar induction of anti-OVA IFN-γ⁺CD8⁺ T-cells and anti-OVA IFN-γ⁺CD8⁺ T-cells after SC OVA plus MVAΔC7L-hFlt3L-TK(−)-mOX40L is predicted to be observed in the dLNs. Briefly, single cell suspensions are generated from dLNs, and 500,000 cells are incubated with either OVA 257-264 or OVA 323-339 peptides. It is further anticipated that the combined administration of MVAΔC7L-hFlt3L-TK(−)-mOX40L and Heat-iMVA with OVA will produce synergistic effects in this regard as compared to the administration of MVAΔC7L-hFlt3L-TK(−)-mOX40L with OVA alone.

### Example 28: MVAΔC7L-hFlt3L-TK(−)-mOX40L is Superior to Complete Freund Adjuvant (CFA) in Generating Antigen-Specific CD8⁺ and CD4⁺ T-Cell Responses

Complete Freund adjuvant (CFA) comprises heat-killed Mycobacterium tuberculosis in non-metabolizable oils (paraffin oil and mannide monooleate). It also contains ligands for TLR2, TLR4, and TLR9. Injection of antigen with CFA induces a Th1-dominant immune response. CFA's use in humans is currently impermissible due to its toxicity profile, and its use in animals is limited to subcutaneous or intraperitoneal routes due to painful reactions and risks of tissue damage at the site of injection. To test whether MVAΔC7L-hFlt3L-TK(−)-mOX40L is superior to CFA, mice are vaccinated subcutaneously with OVA antigen plus MVAΔC7L-hFlt3L-TK(−)-mOX40L or OVA plus CFA twice, 2 weeks apart, and subsequently harvested spleens, dLNs, and blood are harvested for anti-OVA CD8⁺ and CD4⁺ T-cell and antibody responses as described in Example 27.

It is anticipated that subcutaneous co-administration of OVA with MVAΔC7L-hFlt3L-TK(−)-mOX40L will induce higher levels of antigen-specific CD8⁺ and CD4⁺ T-cells compared with immunization with OVA plus CFA in the spleens of vaccinated mice.

### Example 29: MVAΔC7L-hFlt3L-TK(−)-mOX40L is Superior to Poly I:C or STING Agonist in Generating Antigen-Specific CD8⁺ and CD4⁺ T-Cell Responses

Poly IC and STING agonist are innate immune activators that have been investigated as vaccine adjuvants. To test whether MVAΔC7L-hFlt3L-TK(−)-mOX40L is superior to Poly IC and STING agonist, mice are vaccinated subcutaneously with OVA antigen plus MVAΔC7L-hFlt3L-TK(−)-mOX40L or OVA plus Poly IC and STING agonist twice, 2 weeks apart, and subsequently harvested spleens, dLNs, and blood are harvested for anti-OVA CD8⁺ and CD4⁺ T-cell and antibody responses as described in Example 27.

It is anticipated that subcutaneous co-administration of OVA with MVAΔC7L-hFlt3L-TK(−)-mOX40L will induce higher levels of antigen-specific CD8⁺ and CD4⁺ T-cells compared with immunization with OVA plus Poly IC and STING agonist in the spleens of vaccinated mice.

### Example 30: Skin Scarification with MVAΔC7L-hFlt3L-TK(−)-mOX40L-OVA Generates Stronger OVA-Specific CD8⁺ and CD4⁺ T Cells and Antibody Compared with MVA-OVA

MVA is a highly attenuated, non-replicative, safe, and efficacious vaccine vector for various infectious agents and cancers. The optimal dosage for MVA vaccination is tested via skin scarification. MVA-OVA (which encodes full-length of OVA under the control of P7.5 promoter) or MVAΔC7L-hFlt3L-TK(−)-mOX40L-OVA at doses of 10⁵, 10⁶, and 10⁷ pfu are administered to the tails of 6-8 week old female C57BL/6J mice after skin scarification. One week after vaccination, mice are euthanized and the spleens are isolated for testing antigen-specific CD8⁺ T-cell responses. Bone marrow-derived DCs (BMDCs) are infected with MVA-OVA at MOI of 5 for 1 h and then incubated for 5 h before the BMDCs are incubated with splenocytes for 12 h. Cells are processed for intracellular cytokine staining (ICS) for IFN-γ⁺CD8⁺ T-cells. Alternatively, BMDCs are incubated the SIINFEKL peptide (SEQ ID NO: 15) for 1 h and then incubated with splenocytes for 12 h. ICS is performed for IFN-γ⁺CD8⁺ T-cells reactive to SIINFEKL peptide (SEQ ID NO: 15).

To test whether STING or Batf3-dependent DCs, OX40 play a role in MVAΔC7L-hFlt3L-TK(−)-mOX40L-OVA-induced vaccination effects, MVAΔC7L-hFlt3L-TK(−)-mOX40L-OVA at a dose of 10⁶ pfu is also administered to the tails of STING^{Gt/Gt}, or Batf3^{-/-}, or OX40^{-/-} mice after skin scarification. It is anticipated that this example will demonstrate that MVAΔC7L-hFlt3L-TK(−)-mOX40L-OVA is an improved vaccine vector compared with MVA-OVA, and its function requires STING, Batf3-dependent DCs, and OX40-OX40L interaction.

### Example 31: MVAΔC7L-hFlt3L-TK(−)-mOX40L Induces MHC-I Expression of GM-CSF-Cultured Bone Marrow-Derived Dendritic Cells (BMDCs), but it does not Increase Phagocytosis of Antigen

Infection of BMDCs with MVAΔC7L-hFlt3L-TK(−)-mOX40L induces DC maturation that is dependent on the STING-mediated cytosolic DNA-sensing pathway (Dai et al., Science Immunology 2017). In this example, the induction of MHC-I expression on the cell surface of BMDCs by MVAΔC7L-hFlt3L-TK(−)-mOX40L is compared with poly I:C. BMDCs are incubated with OVA in the presence or absence of MVAΔC7L-hFlt3L-TK(−)-mOX40L for 3 or 16 h, or with poly IC for 16 h. The cell surface MHC-I (H-2Kb) expression is determined by FACS using anti-H-2Kb antibody. It is anticipated that co-incubation with MVAΔC7L-hFlt3L-TK(−)-mOX40L will increase the cell surface expression of H-2Kb. It is anticipated that the results will demonstrate that MVAΔC7L-hFlt3L-TK(−)-mOX40L is a stronger inducer of MHC-I expression on BMDCs compared with poly IC.

To assess whether BMDCs' capacity for uptake of fluorescent-labeled model antigen OVA (OVA-647) is affected by MVAΔC7L-hFlt3L-TK(−)-mOX40L treatment, BMDCs are infected with MVAΔC7L-hFlt3L-TK(−)-mOX40L (MOI of 1) for 1 h and then incubated with OVA-647 for 1 h. The fluorescence intensities of phagocytosed OVA-647 in BMDC are measured by flow cytometry. It is anticipated that the results of this experiment will demonstrate that although MVAΔC7L-hFlt3L-TK(−)-mOX40L-treated BMDCs undergo maturation, their capacity to phagocytose antigen is reduced as a consequence of maturation.

### Example 32: Co-Incubation of GM-CSF-Cultured BMDCs with MVAΔC7L-hFlt3L-TK(−)-mOX40L and OVA Enhances Proliferation of OT-I and OT-II T-Cells In Vitro

Infection of epidermal dendritic cells with live WT vaccinia inhibits DCs' capacity to activate antigen-specific T-cells (Deng et al., JVI, 2006). To test whether MVAΔC7L-hFlt3L-TK(−)-mOX40L infection of BMDCs enhances the proliferation of antigen-specific OT-I and OT-II T-cells, BMDCs are incubated with OVA at various concentrations in the presence or absence of MVAΔC7L-hFlt3L-TK(−)-mOX40L for 3 h. Cells are washed to remove unabsorbed OVA or virus, and then co-cultured with Carboxyfluorescein Diacetate Succinimidyl Ester (CFSE)-labeled OT-I T-cells for 3 days (BMDC:OT-I T-cells=1:5). Flow cytometry is applied to measure CFSE intensities of OT-I cells. It is anticipated that pre-incubation with MVAΔC7L-hFlt3L-TK(−)-mOX40L will enhance the capacity of DCs to stimulate the proliferation of OT-I T-cells, as indicated by CSFE dilution in dividing cells. It is also anticipated that pre-treatment with MVAΔC7L-hFlt3L-TK(−)-mOX40L or poly IC enhances DCs' capacity to stimulate the proliferation of OT-II T-cells that recognize OVA-antigen presented by MHC-II on DCs. It is further anticipated that the combined administration of MVAΔC7L-hFlt3L-TK(−)-mOX40L and Heat-iMVA will produce synergistic effects in this regard as compared to the administration of MVAΔC7L-hFlt3L-TK(−)-mOX40L alone.

### Example 33: Co-Incubation of Flt3L-Cultured BMDCs with MVAΔC7L-hFlt3L-TK(−)-mOX40L and OVA Enhances the Proliferation of OT-I T-Cells In Vitro

FMS-like tyrosine kinase 3 ligand (Flt3L) is a critical growth factor for the differentiation of Batf3-dependent CD103⁺/CD8□⁺ DCs and plasmacytoid DCs (pDCs). Flt3L-cultured BMDCs are pulsed with OVA in the presence or absence of MVAΔC7L-hFlt3L-TK(−)-mOX40L, and then co-cultured with CFSE-labeled OT-I cells for 3 days (BMDC:OT-I=1:5). Flow cytometry is applied to measure CFSE intensities of OT-I cells. It is anticipated that MVAΔC7L-hFlt3L-TK(−)-mOX40L will stimulate the proliferation of OT-I cells, which recognizes OVA₂₅₇₋₂₆₄ (SIINFEKL) peptide (SEQ ID NO: 15) presented on MHC-I, even at very low concentrations of OVA. It is further anticipated that the combined administration of MVAΔC7L-hFlt3L-TK(−)-mOX40L and Heat-iMVA will produce synergistic effects in this regard as compared to the administration of MVAΔC7L-hFlt3L-TK(−)-mOX40L alone.

### Example 34: Plasmacytoid Dendritic Cells (pDCs) Play Important Role in MVAΔC7L-hFlt3L-TK(−)-mOX40L-Mediated Vaccine Adjuvant Effects

Plasmacytoid DCs (pDCs) can cross-present antigen to stimulate CD8⁺ T-cell responses. To test whether pDCs play a role in MVAΔC7L-hFlt3L-TK(−)-mOX40L-mediated adjuvant effect in vivo, anti-PDCA-1 antibody is used one day prior and one day post intradermal immunization with OVA+MVAΔC7L-hFlt3L-TK(−)-mOX40L, which are performed on Day 0 and Day 14. Spleens and dLNs are isolated on day 21 for antigen-specific CD8⁺ T-cell analyses. It is anticipated that intradermal co-administration of OVA+MVAΔC7L-hFlt3L-TK(−)-mOX40L increases the percentage of IFN-□⁺ T-cells among CD8⁺ T-cells in the spleens. It is also anticipated that depletion of pDCs results in a decrease in the percentage of IFN-□⁺ T-cells among CD8⁺ T-cells in the spleens. The results of this experiment are anticipated to demonstrate the role of pDCs in MVAΔC7L-hFlt3L-TK(−)-mOX40L-elicited vaccine adjuvant effects in a peptide vaccination model in vivo.

### Example 35: Migratory Dendritic Cell Subsets Langerin-CD11b- and CD11b⁺ DCs are Efficient in OVA Antigen Uptake

Many DC subsets are present in the lymph nodes, which include migratory DCs and resident DCs. Migratory DCs are MHC-II⁺CD11c⁺. Resident dendritic cell populations are MHC-II^{Int}CD11c⁺. Migratory DCs can be further separated into CD11b⁺ DC, Langerin CD11b⁻ DC, and Langerin⁺ DC. Langerin⁺ DCs comprise of CD103⁺ DC and Langerhans cells, whereas resident DCs are composed of CD8α⁺ resident DC and CD8α⁻ resident DC. To test which DCs subsets are efficient in phagocytosing OVA antigen labeled with fluorescent dye (OVA-647) and have the capacity to migrate to the dLNs, OVA-647 are injected intradermally (ID) to the right flank and harvested the dLNs at 24 h post injection. To compare whether co-administration of OVA-647 with or without vaccine adjuvants Addavax or MVAΔC7L-hFlt3L-TK(−)-mOX40L affects the percentages of OVA-647⁺ cells among Langerin⁻CD11b⁻ and CD11b⁻ DCs, OVA-647 is intradermally (ID) injected with or without Addavax or MVAΔC7L-hFlt3L-TK(−)-mOX40L, and analyzed OVA-647⁺ DCs among Langerin⁻CD11b⁻ and CD11b⁺ DCs. It is anticipated that co-administration of OVA with MVAΔC7L-hFlt3L-TK(−)-mOX40L increases the percentages of OVA-647⁺ cells among Langerin⁻CD11b⁻ and CD11b⁺ DCs, whereas co-administration of OVA with Addavax fails to do the same. Addavax is a well-accepted squalene-based oil-in-water nano-emulsion with a formulation similar to MF59 that has been licensed in Europe for adjuvanted flu vaccines. It is anticipated that the results of this experiment will suggest that co-administration of OVA-647 with MVAΔC7L-hFlt3L-TK(−)-mOX40L enhances migratory DCs' capacity to transport phagocytosed antigen to the dLNs. It is further anticipated that the combined administration of MVAΔC7L-hFlt3L-TK(−)-mOX40L and Heat-iMVA will produce synergistic effects in this regard as compared to the administration of MVAΔC7L-hFlt3L-TK(−)-mOX40L alone.

### Example 36: MVAΔC7L-hFlt3L-TK(−)-mOX40L is a Potent Immune Adjuvant for Irradiated Whole Cell Vaccine

The advantage of using irradiated whole cell vaccines rather than peptide tumor antigen or neoantigen include: (i) tumor cells provide multiple tumor antigens that can be recognized by the host immune system; and (ii) can bypass the need or time to identify tumor antigens or neoantigens. Whether the addition of MVAΔC7L-hFlt3L-TK(−)-mOX40L with irradiated B16-OVA improves vaccination efficacy, and whether systemic delivery of anti-PD-L1 would further improve vaccination efficacy is analyzed. Mice are intradermally implanted with B16-OVA, they are vaccinated intradermally with irradiated B16-OVA, B16-OVA+MVAΔC7L-hFlt3L-TK(−)-mOX40L, or B16-OVA+poly IC three times at day 3, 6, and 9 on the contralateral flank.

It is anticipated that vaccination with irradiated B16-OVA+MVAΔC7L-hFlt3L-TK(−)-mOX40L will extend the median survival vs. Irradiated B16-OVA alone. It is also anticipated that, in the presence of anti-PD-L1 antibody, vaccination with irradiated B16-OVA+MVAΔC7L-hFlt3L-TK(−)-mOX40L will extend the median survival vs. Irradiated B16-OVA+anti-PD-L1. It is anticipated that these results will demonstrate that MVAΔC7L-hFlt3L-TK(−)-mOX40L is a potent and safe vaccine adjuvant for irradiated whole cell vaccination.

### Example 37: MVAΔC7L-hFlt3L-TK(−)-mOX40L is an Immune Adjuvant for Neoantigen Peptide Vaccination

To test whether MVAΔC7L-hFlt3L-TK(−)-mOX40L can act as a vaccine adjuvant for neoantigen peptide vaccination, a subcutaneous vaccination model was used in which mice are first implanted with B16-F10 cells (7.5×10⁴ cells per mouse) intradermally. At day 3, 7, and 10 post implantation, mice are vaccinated at the contralateral flank subcutaneously (SC) with either a mixture of neoantigen peptides (M27 (REGVELCPGNKYEMRRHGTTHSLVIHD) (SEQ ID NO: 17), M30 (PSKPSFQEFVDWENVSPELNSTDQPFL) (SEQ ID NO: 18), and M48 (SHCHWNDLAVIPAGVVHNWDFEPRKVS) (SEQ ID NO: 19)) with or without either MVAΔC7L-hFlt3L-TK(−)-mOX40L or poly I:C. Tumor growth and mice survival are monitored. It is anticipated that SC vaccination with neoantigen peptides alone generates systemic antitumor immunity and the antitumor effect is enhanced when neoantigen peptide mix are co-administered with MVAΔC7L-hFlt3L-TK(−)-mOX40L.

### Example 38: MVAΔC7L-hFlt3L-TK(−)-mOX40L is an Immune Adjuvant for Viral Antigen Peptide Vaccination

Viral antigens are potent immunogens that can be recognized by the host immune system. To test whether the combination of MVAΔC7L-hFlt3L-TK(−)-mOX40L and viral antigen (such as synthetic long peptide (SLP) of human papilloma virus E7) elicits antiviral T cells, mice will be subcutaneously vaccinated with E7 SLP alone, or E7 SLP plus MVAΔC7L-hFlt3L-TK(−)-mOX40L, or E7 plus poly I:C twice, two weeks apart, and spleens are subsequently harvested, dLNs, and blood are harvested for anti-CD8⁺ and CD4⁺ T-cell and antibody responses. To test the role of MVAΔC7L-hFlt3L-TK(−)-mOX40L in the therapeutic vaccination model, E7-expressing cancer cells (TC-1) will be implanted intradermally, and then the vaccination will be performed with or without adjuvant two weeks apart, and tumor volumes and mice survival will be followed.

### Example 39: Skin Scarification with MVAΔC7L-hFlt3L-TK(−)-mOX40L-E7 Generates a Stronger Anti-E7 CD8+ and CD4+ T Cell Responses Compared with MVA-E7

Recombinant MVAΔC7L-hFlt3L-TK(−)-mOX40L virus expressing HPV E7 gene will be generated by inserting HPV E7 gene under the control of vaccinia psE/L promoter into MVA E5R or K7R loci. MVA-E7 (which encodes full-length of HPV E7 under the control of psE/L promoter inserted in the TK locus) or MVAΔC7L-hFlt3L-TK(−)-mOX40L-E7 at doses of 10⁶ or 10⁷ pfu are administered to the tails of 6-8 week old female C57BL/6J mice after skin scarification. One week after vaccination, mice are euthanized and the spleens are isolated for testing antigen-specific CD8⁺ T-cell responses. Bone marrow-derived DCs (BMDCs) are infected with MVA-E7 at MOI of 5 for 1 h and then incubated for 5 h before the BMDCs are incubated with splenocytes for 12 h. Cells are processed for intracellular cytokine staining (ICS) for IFN-γ⁺CD8⁺ T-cells. Alternatively, BMDCs are incubated the E7 peptide for 1 h and then incubated with splenocytes for 12 h. ICS is performed for IFN-γ⁺CD8⁺ T-cells reactive to E7 peptide.

### Example 40: Intranasal Infection of MVAΔC7L-hFlt3L-TK(−)-mOX40L-E7 Provides Better Protection of Mice from TC-1 Cells (Expressing E7) Growth in the Lungs Compared with MVA-E7

Six-eight week-old female C57BL/6J mice are intranasally infected with MVAΔC7L-hFlt3L-TK(−)-mOX40L-E7, or MVA-E7 (at 2×10⁷ pfu), or PBS control. One week after intranasal infection, mice are challenged with 1×10⁵ TC-1 cells through tail-vein injection. Mice are euthanized 3 weeks later to evaluate tumor growth in the lungs. It is anticipated that vaccination with MVAΔC7L-hFlt3L-TK(−)-mOX40L-E7 provides better protection against E7-expressing tumor cell growth in the lungs compared with MVA-E7.

### Example 41: Test Whether Intratumoral (IT) Vaccination is Superior to Subcutaneous (SC) Vaccination in Generating Antigen-Specific Immune Responses

Rationale: It was previously shown that intratumoral (IT) injection of Heat-iMVA eradicates injected tumors and induces systemic antitumor immunity, which requires Batf3-dependent CD103⁺/CD8□⁺ DCs and STING-mediated cytosolic DNA-sensing pathway. IT delivery of Heat-iMVA alters tumor immunosuppressive microenvironment partially through activating cGAS/STING pathway and promotes tumor antigen presentation by the CD103⁺ DCs. It is hypothesized that IT delivery of Heat-iMVA plus model antigen or neoantigen would enhance antigen presentation by tumor-infiltrating DCs and generate superior adaptive immunity compared with SC delivery of Heat-iMVA plus antigen.

Methods: To test whether IT vaccination is superior to SC vaccination in generating antigen-specific immune responses, B16-F10 melanoma cells (5×10⁵ cells) will be intradermally implanted at the right flank. At day 7 post implantation, when the tumors are 2-3 mm in diameter, MVAΔC7L-hFlt3L-TK(−)-mOX40L and OVA protein will either be directly injected into the tumors or injected SC 1 cm away from the tumors on the right flank. At one week post injection, TDLNs and spleens will be collected and anti-OVA CD4 and CD8 T cells will be analyzed by FACS.

Alternatively, B16-F10 neoantigen peptide mix (M27/M30/M48) will be co-injected with MVAΔC7L-hFlt3L-TK(−)-mOX40L either directly into the tumors on the right flank, or injected SC 1 cm away from the tumors on the right flank. At one week post injection, TDLNs and spleens will be collected and co-cultured with either M27 (REGVELCPGNKYEMRRHGTTHSLVIHD) (SEQ ID NO: 17), M30 (PSKPSFQEFVDWENVSPELNSTDQPFL) (SEQ ID NO: 18), or M48 (SHCHWNDLAVIPAGVVHNWDFEPRKVS) (SEQ ID NO: 19) peptide for 16 h for ELISPOT analysis.

### Example 42: Test Whether Intratumoral (IT) Vaccination is Superior to Subcutaneous (SC) Vaccination in Generating Antigen-Specific Immune Responses in the Presence of Immune Checkpoint Blockade

To test whether IT vaccination is superior to SC vaccination in generating antigen-specific immune responses in the presence of immune checkpoint blockade antibodies, including anti-CTLA-4, anti-PD-1, or anti-PD-L1, B16-F10 melanoma cells (5×10⁵ cells) will be intradermally implanted at the right flank. At day 7 post implantation, when the tumors are 2-3 mm in diameter, MVAΔC7L-hFlt3L-TK(−)-mOX40L and OVA protein will either be directly injected into the tumors or injected SC 1 cm away from the tumors on the right flank twice, three days apart. Anti-CTLA-4, anti-PD-1, or anti-PD-L1, or isotype control antibody will be administered intraperitoneally twice, three days apart. At 2 days post second injection, TDLNs and spleens will be collected and anti-OVA CD4 and CD8 T cells will be analyzed by FACS.

Alternatively, B16-F10 neoantigen peptide mix (M27/M30/M48) will be co-injected with MVAΔC7L-hFlt3L-TK(−)-mOX40L either directly into the tumors on the right flank, or injected SC 1 cm away from the tumors on the right flank twice, three days apart. Anti-CTLA-4, anti-PD-1, or anti-PD-L1, or isotype control antibody will be administered intraperitoneally twice, three days apart. At 2 days post second injection, TDLNs and spleens will be collected and co-cultured with either M27, M30, or M48 peptide for 16 h for ELISPOT analysis.

### Example 43: E3LΔ83N-TK⁻-hFlt3L-Anti-muCTLA-4/C7L⁻-mOX40L or VAC-TK⁻-Anti-muCTLA-4/C7L⁻-mOX40L Recombinant Viruses are Replication Competent

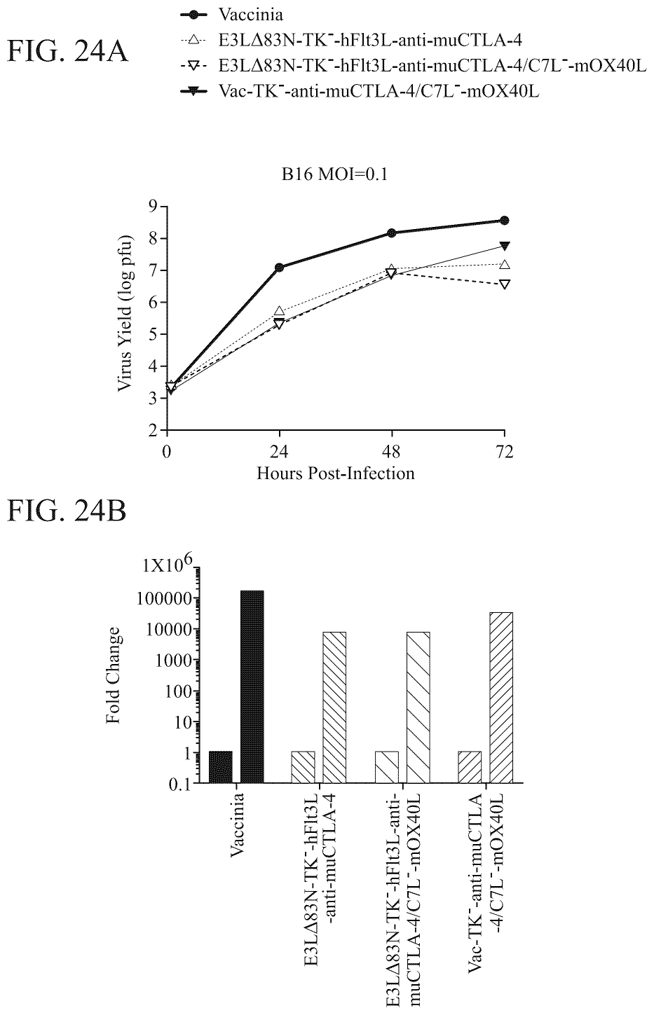

The replication capacities of E3LΔ83N-TK⁻-hFlt3L-anti-muCTLA-4/C7L⁻-mOX40L or VAC-TK⁻-anti-muCTLA-4/C7L⁻-mOX40L were determined in murine B16-F10 melanoma cells by infecting them at a MOI of 0.1. Cells were collected at various time points post infection (e.g., 1, 24, 48, and 72 h) and viral yields (log pfu) were determined by titrating on BSC40 cells. FIG. 24A shows the graphs of viral yields plotted against hours post infection. Both E3LΔ83N-TK⁻-hFlt3L-anti-muCTLA-4/C7L⁻-mOX40L and VAC-TK⁻-anti-muCTLA-4/C7L⁻-mOX40L replicated efficiently in B16-F10 cells with viral titers increasing by more than 1421 and 32142-fold at 72h post-infection, respectively. The fold changes of viral yields at 72 h over those at 1 h post infection were calculated (FIG. 24B). The recombinant viruses E3LΔ83N-TK⁻-hFlt3L-anti-muCTLA-4/C7L⁻-mOX40L and VAC-TK⁻-anti-muCTLA-4/C7L⁻-mOX40L have replicated efficiently in murine B16-F10 melanoma cells.

TABLE 6
Quantitative data (fold change at 72 hpi)
for results shown in FIGS. 24A and 24B.

| | | | E3LΔ83N-TK- | |
|---|---|---|---|---|
| | | E3LΔ83N-TK- | hFlt3L-anti- | VAC-TK-anti- |
| | | hFlt3L-anti- | muCTLA- | muCTLA-4/C7L- |
| | Vaccinia | muCTLA-4 | 4/C7L mOX40L | mOX40L |
| B16-F10 | 161111 | 7052 | 1421 | 32142 |

### Example 44: Expression of Anti-muCTLA-4, hFlt3L, and mOX40L in B16-F10 Melanoma Cells Via Infection of E3LΔ83N-TK⁻-hFlt3L-Anti-muCTLA-4/C7L⁻-mOX40L Virus

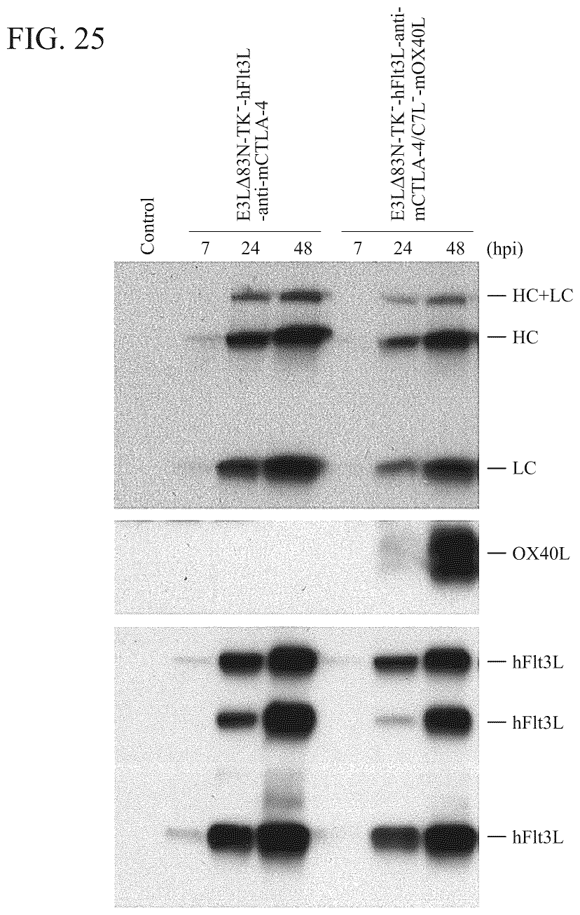

To determine whether E3LΔ83N-TK⁻-hFlt3L-anti-muCTLA-4/C7L⁻-mOX40L recombinant viruses are capable of expressing desired specific genes, B16-F10 murine melanoma cells were infected with E3LΔ83N-TK⁻-hFlt3L-anti-muCTLA-4 or E3LΔ83N-TK⁻-hFlt3L-anti-muCTLA-4/C7L⁻-mOX40L at a MOI of 10, and the expression of anti-muCTLA-4, hFlt3L and mOX40L was measured. Cell lysates were collected at various times (e.g., 7, 24, and 48 h) post infection. Western blot analyses were performed to determine the levels of the antibodies and proteins. As shown in FIG. 25, there is abundant expression of anti-muCTLA-4 antibody (Heavy Chain (HC) and Light Chain (LC)), hFlt3L, and mOX40L protein in B16-F10 cells infected with E3LΔ83N-TK⁻-hFlt3L-anti-muCTLA-4/C7L⁻-mOX40L virus. Accordingly, these results demonstrate that the recombinant viruses of the present technology have the capacity to simultaneously express multiple specific genes of interest from different loci in the virus in infected cells and are useful in methods for delivering the desired products to cells.

### Example 45: Cell Surface Expression of mOX40L in B16-F10 Melanoma Cells Via Infection of E3LΔ83N-TK⁻-hFlt3L-Anti-muCTLA-4/C7L⁻-mOX40L or VAC-TK⁻-Anti-muCTLA-4/C7L⁻-mOX40L Viruses

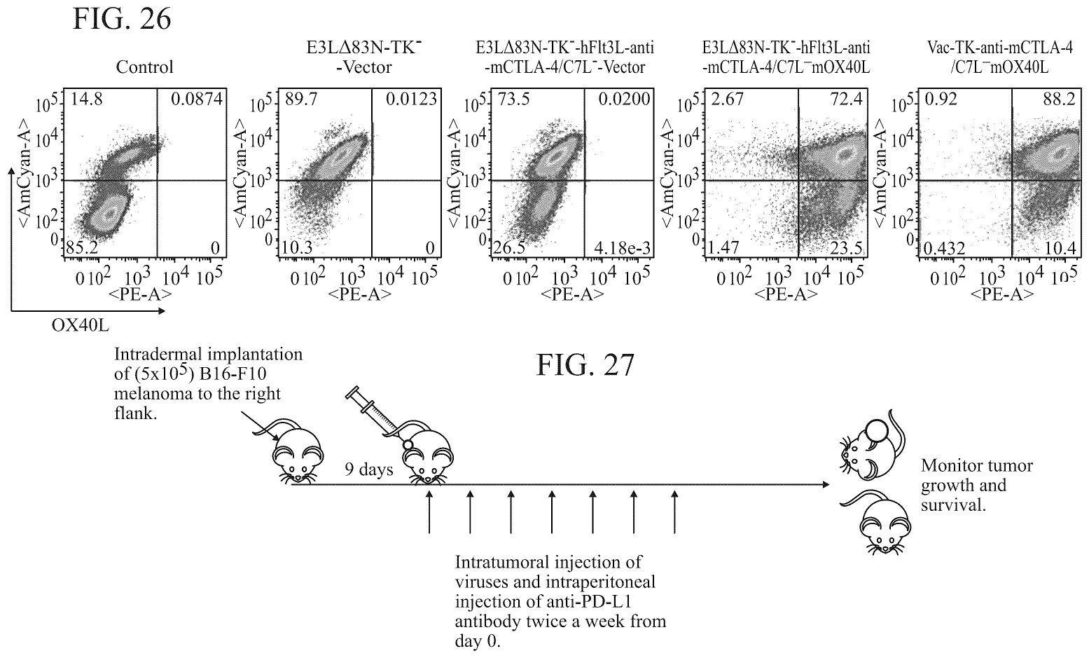

To determine whether mOX40L is expressed on the surface of murine B16-F10 cells infected with E3LΔ83N-TK⁻-hFlt3L-anti-muCTLA-4/C7L⁻-mOX40L or VAC-TK⁻-anti-muCTLA-4/C7L⁻-mOX40L recombinant viruses, B16-F10 cells were infected with E3LΔ83N-TK⁻-hFlt3L-anti-muCTLA-4, E3LΔ83N-TK⁻-hFlt3L-anti-muCTLA-4/C7L⁻-mOX40L, or VAC-TK⁻-anti-muCTLA-4/C7L⁻-mOX40L at a MOI of 10. The cell surface mOX40L expression is determined by FACS using anti-mOX40L antibody at 24 h post infection. As shown in FIG. 26, there is abundant cell surface expression of mOX40L protein in murine B16-F10 cells infected with E3LΔ83N-TK⁻-hFlt3L-anti-muCTLA-4/C7L⁻-mOX40L or VAC-TK⁻-anti-muCTLA-4/C7L⁻-mOX40L viruses. Accordingly, these results demonstrate that the recombinant viruses of the present technology have the capacity to express specific genes of interest in infected cells and are useful in methods for delivering the desired products to cells and the cell surface.

### Example 46: The Combination with IT Delivery of MVAΔC7L-hFlt3L-TK(−)-mOX40L and Systemic Delivery of Anti-PD-L1 Antibody Significantly Increases the Overall Responses and Cure Rate in B16-F10 Melanoma Unilateral Implantation Model

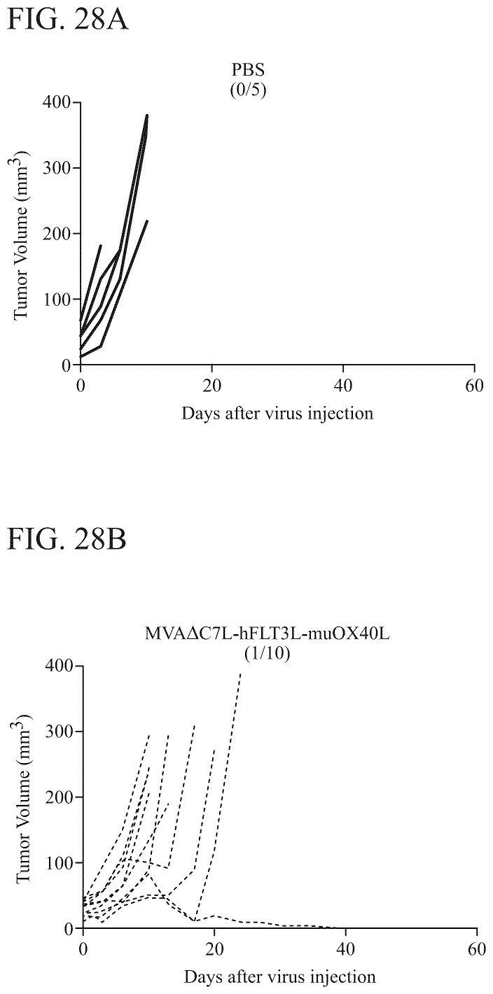

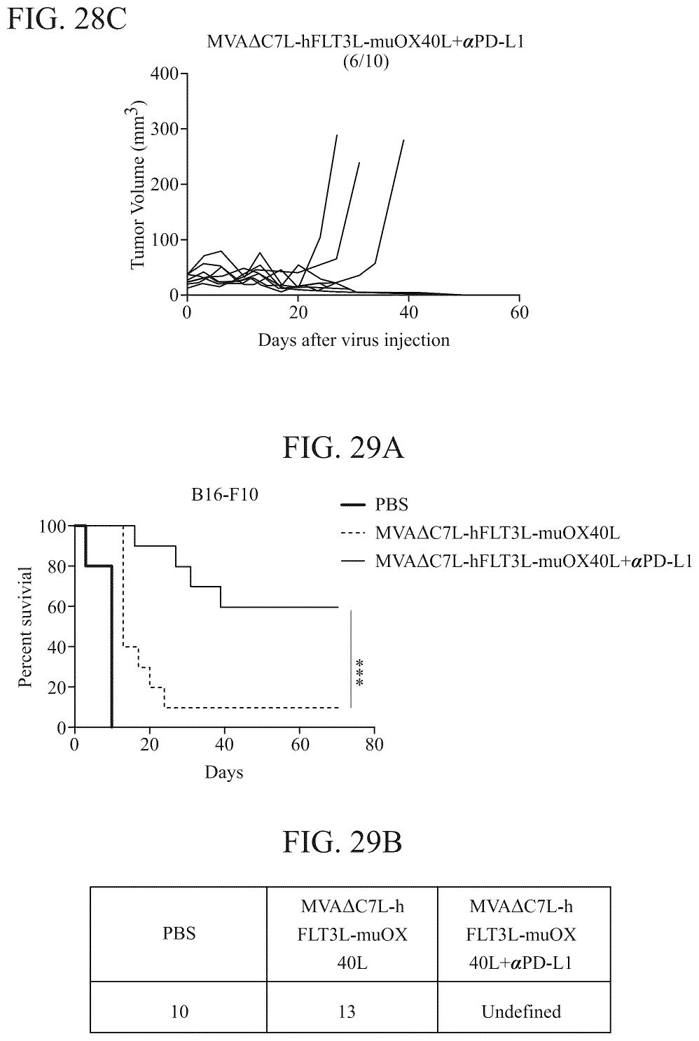

The combination with IT delivery of MVAΔC7L-hFlt3L-TK(−)-mOX40L and systemic delivery of anti-PD-L1 demonstrated superior anti-tumor efficacy in a murine B16-F10 melanoma unilateral implantation model. Briefly, 5×10⁵ B16-F10 melanoma cells were implanted intradermally into the shaved skin on the right flanks of C57BL/6J mice. 9 days post implantation, the tumors were injected twice a week with PBS or 4×10⁷ pfu of MVAΔC7L-hFlt3L-TK(−)-mOX40L. Anti-PD-L1 antibody were given intraperitoneally at 250 μg per mouse (FIG. 27). Tumor volumes of individual mice were measured (FIGS. 28A-28C) and the overall survival rate of mice was monitored (FIGS. 29A-29B). Intratumoral injection of MVAΔC7L-hFlt3L-TK(−)-mOX40L delayed tumor growth and improved survival compared with PBS, with a median survival of 13 days. The combination of IT MVAΔC7L-hFlt3L-TK(−)-mOX40L and IP anti-PD-L1 antibody showed better anti-tumor efficacy in eradicating tumors compared with MVAΔC7L-hFlt3L-TK(−)-mOX40L alone, with 60% of mice tumor-free and survived after treatments.

### Example 47: The Combination with IT Delivery of MVAΔC7L-hFlt3L-TK(−)-mOX40L and Systemic Delivery of Anti-PD-L1 Antibody Significantly Increases the Overall Responses and Cure Rate in MC38 Colon Cancer Unilateral Implantation Model

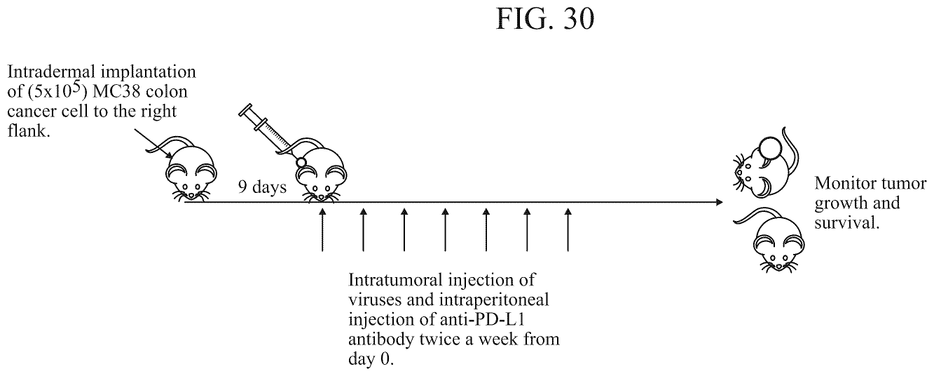

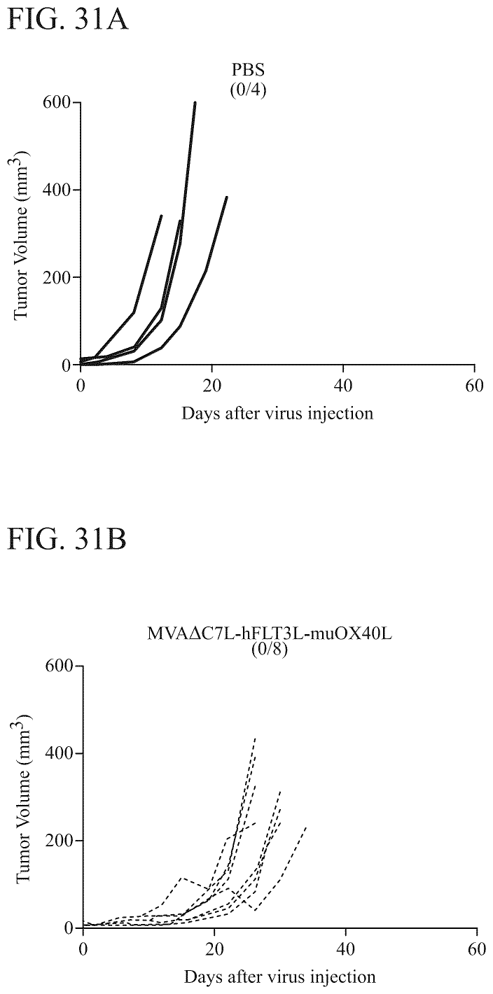

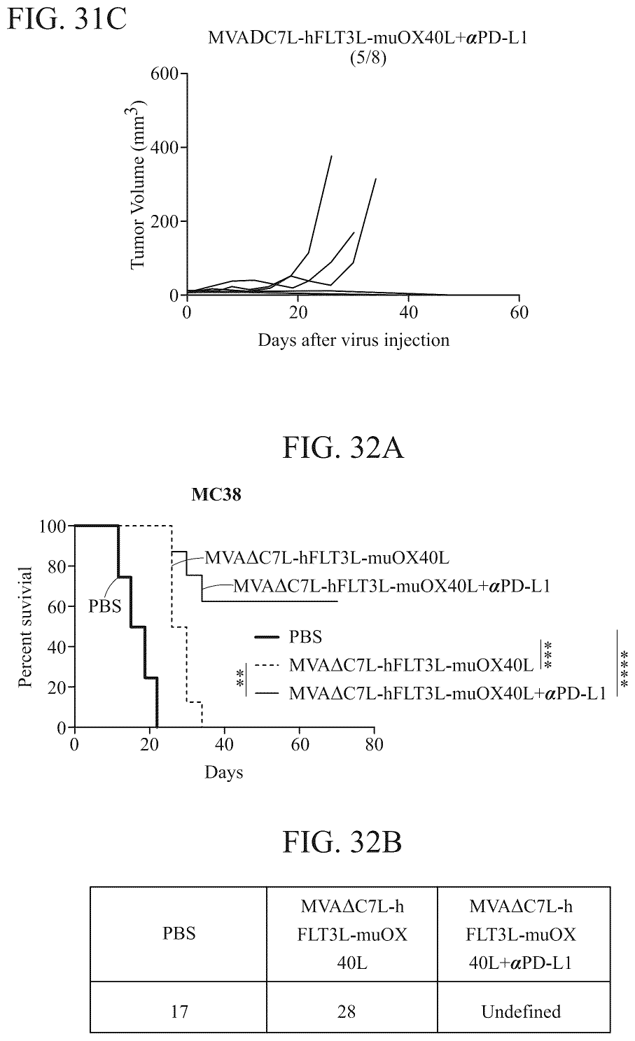

The combination with IT delivery of MVAΔC7L-hFlt3L-TK(−)-mOX40L and systemic delivery of anti-PD-L1 had superior anti-tumor efficacy in MC38 colon cancer unilateral implantation model. Briefly, 5×10⁵ MC38 cells were implanted intradermally into the shaved skin on the right flanks of C57BL/6J mice. 9 days post implantation, the tumors were injected twice a week with PBS or 4×10⁷ pfu of MVAΔC7L-hFlt3L-TK(−)-mOX40L. Anti-PD-L1 antibody were given intraperitoneally at 250 μg per mouse (FIG. 30). Tumor volumes of individual mice were measured (FIGS. 31A-31C) and the overall survival rate of mice was monitored (FIGS. 32A-32B). Intratumoral injection of MVAΔC7L-hFlt3L-TK(−)-mOX40L delayed tumor growth and prolonged survival compared with PBS, with extension of median survival to 28 days. The combination of IT MVAΔC7L-hFlt3L-TK(−)-mOX40L and IP anti-PD-L1 antibody showed better anti-tumor efficacy in eradicating tumors compared with MVAΔC7L-hFlt3L-TK(−)-mOX40L alone, with 62.5% of mice tumor-free and survived after treatments.

### Example 48: The Combination with IT Delivery of MVAΔC7L-hFlt3L-TK(−)-mOX40L and Systemic Delivery of Anti-PD-L1 Antibody Significantly Increases the Overall Responses and Cure Rate in MB49 Bladder Cancer Unilateral Implantation Model

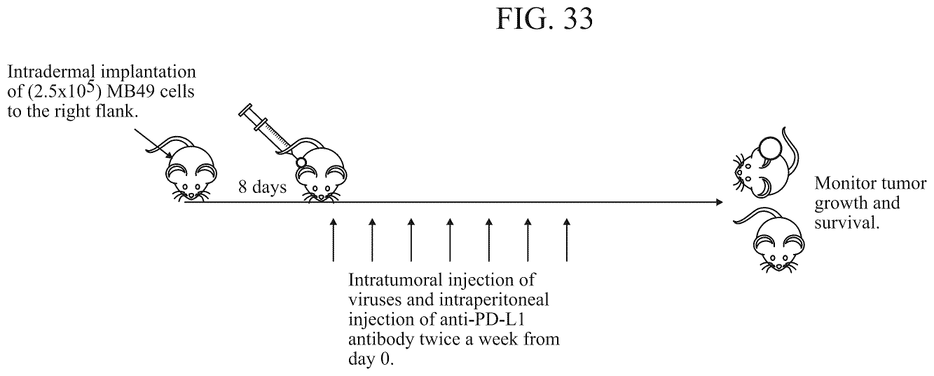

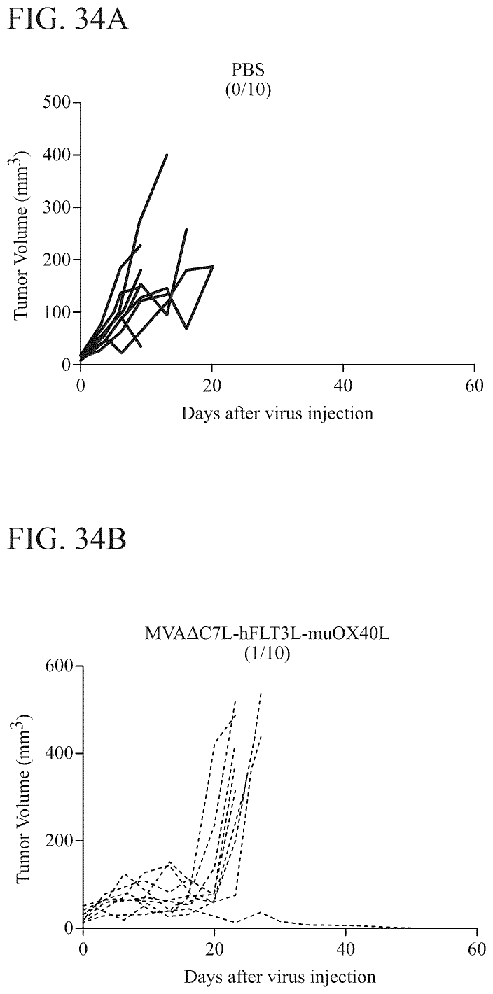

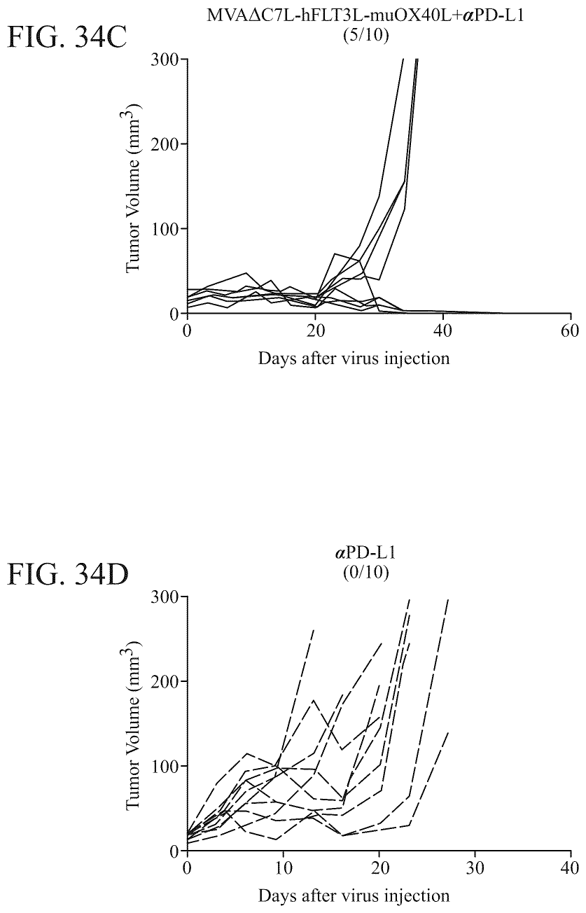

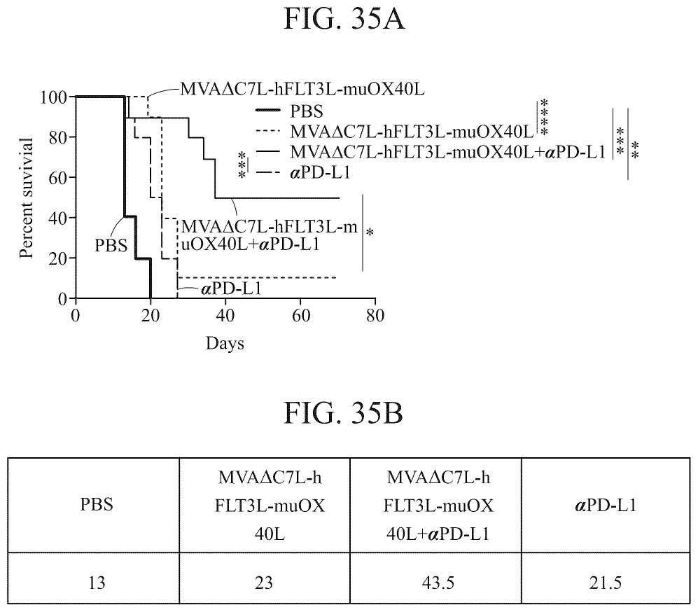

The combination with IT delivery of MVAΔC7L-hFlt3L-TK(−)-mOX40L and systemic delivery of anti-PD-L1 had superior anti-tumor efficacy in MB49 bladder cancer unilateral implantation model. Briefly, 2.5×10⁵ MB49 cells were implanted intradermally into the shaved skin on the right flanks of C57BL/6J mice. 8 days post implantation, the tumors were injected twice a week with PBS or 4×10⁷ pfu of MVAΔC7L-hFlt3L-TK(−)-mOX40L, or the mice were administered anti-PD-L1 antibody twice a week. Anti-PD-L1 antibody were given intraperitoneally at 250 μg per mouse (FIG. 33). Tumor volumes of individual mice were measured (FIGS. 34A-34D) and the overall survival rate of mice was monitored (FIGS. 35A-35B). In mice treated with PBS, the tumors grew rapidly, which resulted in early death with median survival of 13 days. Intratumoral injection of MVAΔC7L-hFlt3L-TK(−)-mOX40L delayed tumor growth and prolonged survival compared with PBS, with extension of median survival to 23 days. IP delivery of anti-PD-L1 antibody alone received partial responses with delayed tumor growth and extended median survival to 21.5 days compared with PBS but mice were unable to reject tumors. The combination of IT MVAΔC7L-hFlt3L-TK(−)-mOX40L and IP anti-PD-L1 antibody are more effective in eradicating MB49 tumors compared with MVAΔC7L-hFlt3L-TK(−)-mOX40L alone or anti-PD-L1 antibody alone with extended median survival to 43.5 days. 50% of mice were tumor-free and survived after treatments

### Example 49: Spontaneous Breast Cancers are Responsive to the Combination Therapy with IT Delivery of MVAΔC7L-hFlt3L-TK(−)-mOX40L and Systemic Delivery of Anti-PD-L1 Antibody

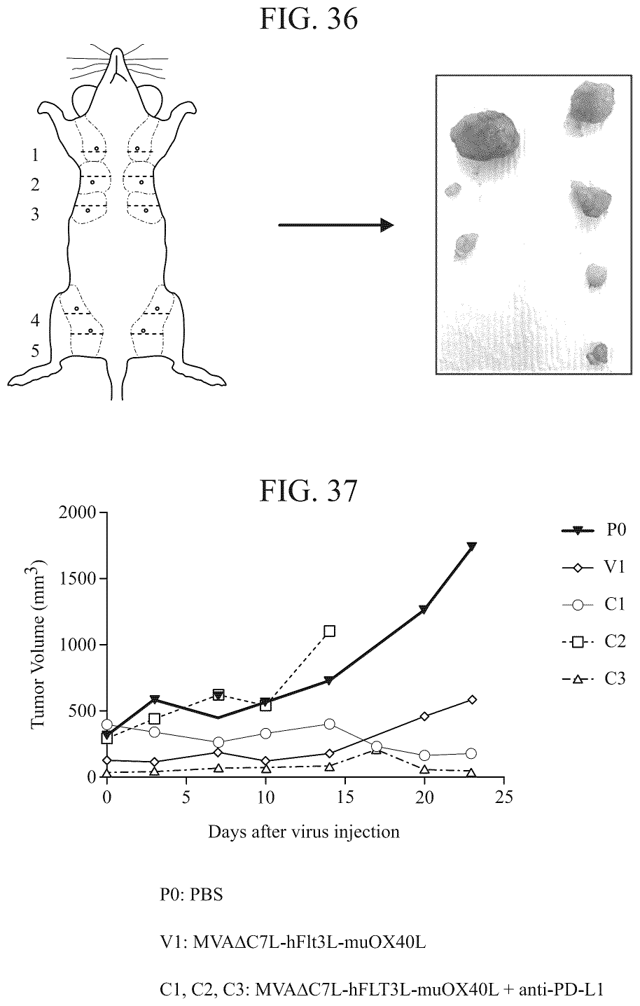

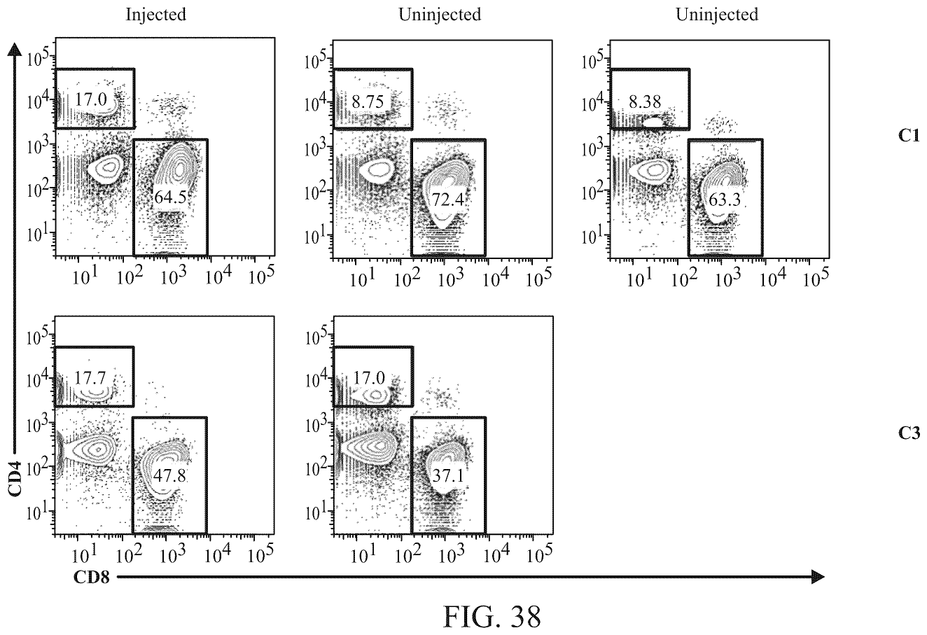

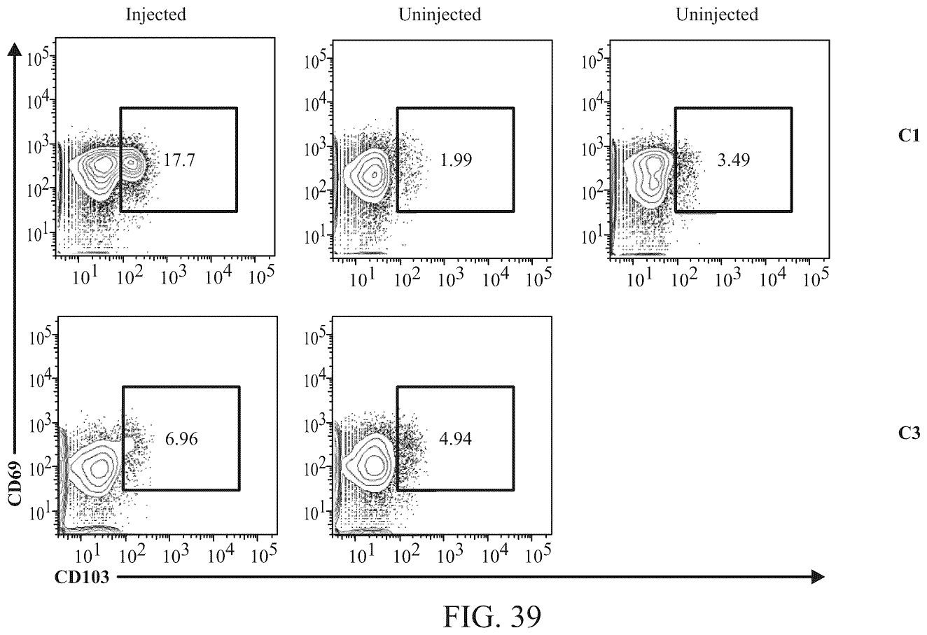

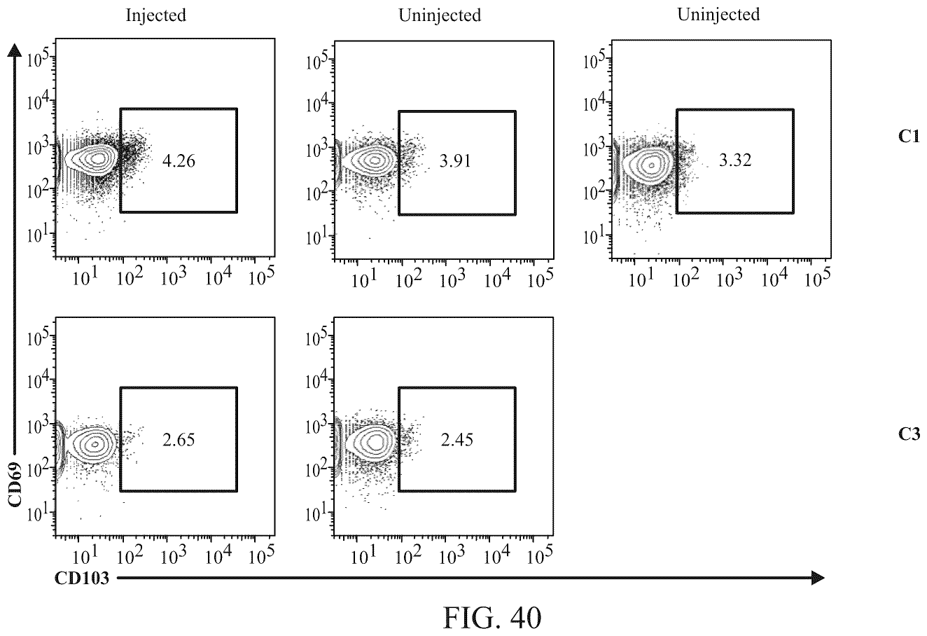

The combination with IT delivery of MVAΔC7L-hFlt3L-TK(−)-mOX40L and systemic delivery of anti-PD-L1 had superior anti-tumor efficacy in spontaneous breast cancers. MMTV-PyMT females develop multiple palpable mammary tumors with mean latency of 92 days of age, which are commonly used as a spontaneous tumor model (FIG. 36). After the first tumor became palpable, MVAΔC7L-hFlt3L-TK(−)-mOX40L was injected to tumors on the right flanks and anti-PD-L1 antibody was given intraperitoneally at 250 μg per mouse. Tumor sizes were measured twice a week. Tumors from 2 mice that received IT MVAΔC7L-hFlt3L-TK(−)-mOX40L and IP anti-PD-L1 antibody treatments were harvested and processed to single cell suspensions for surface labeling with anti-CD45, CD3, CD8, CD4, CD103 and CD69 antibodies. The live tumor infiltrating T cells were analyzed by FACS. Tumor volumes of individual mice were measured (FIG. 37). The mouse treated with PBS developed multiple tumors and the tumors grew rapidly. Intratumoral injection of MVAΔC7L-hFlt3L-TK(−)-mOX40L delayed tumor growth compared with PBS. The combination of IT MVAΔC7L-hFlt3L-TK(−)-mOX40L and IP anti-PD-L1 antibody are more effective in suppressing tumor occurrence and growth compared with MVAΔC7L-hFlt3L-TK(−)-mOX40L alone. FACS analyses of tumor infiltrating lymphocytes showed that T cells were abundant in tumor microenvironment in PyMT tumors (FIG. 38). IT MVAΔC7L-hFlt3L-TK(−)-mOX40L and IP anti-PD-L1 antibody treatment induced a higher percentage of CD103⁺CD69⁺ cell population of CD8⁺ T cells (FIG. 39), which represents activated, memory-like CD8⁺ T cells. IT MVAΔC7L-hFlt3L-TK(−)-mOX40L and IP anti-PD-L1 antibody treatment did not induce a higher percentage of CD103⁺CD69⁺ cell population of CD4⁺ T cells (FIG. 40), demonstrating that the anti-tumor effect of the combination therapy in MMTV-PyMT tumor model mainly relied on CD8⁺ T cell activation.

### Example 50: Generation of B16-F10 Stable Cell Lines Over-Expressing hFlt3L or mOX40L

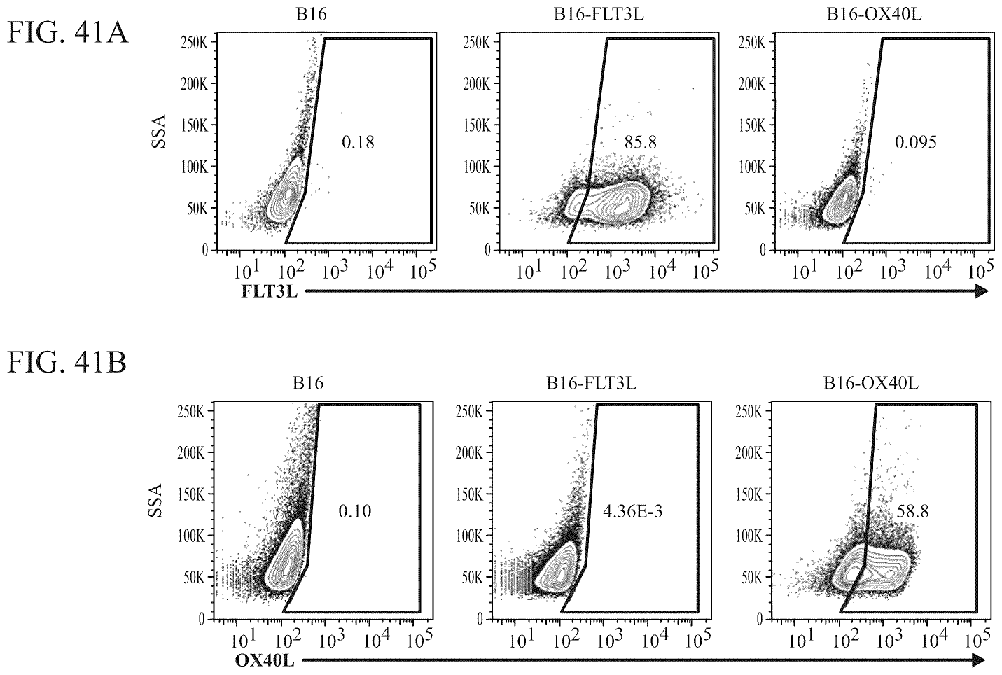

This example demonstrated the generation of B16-F10 stable cell line overexpressing either hFlt3L or mOX40L. Briefly, B16-F10 cells were transfected with retrovirus expressing either hFlt3L or mOX40L. After selection in 2 μg/ml puromycin for one week, cells were harvested and hFlt3L (FIG. 41A) or mOX40L (FIG. 41B) expression at cellular surface were detected and confirmed by FACS.

### Example 51: B16-F10 Melanoma Cells Overexpressing hFlt3L are More Responsive to Intratumoral Delivery of MVAΔC7L

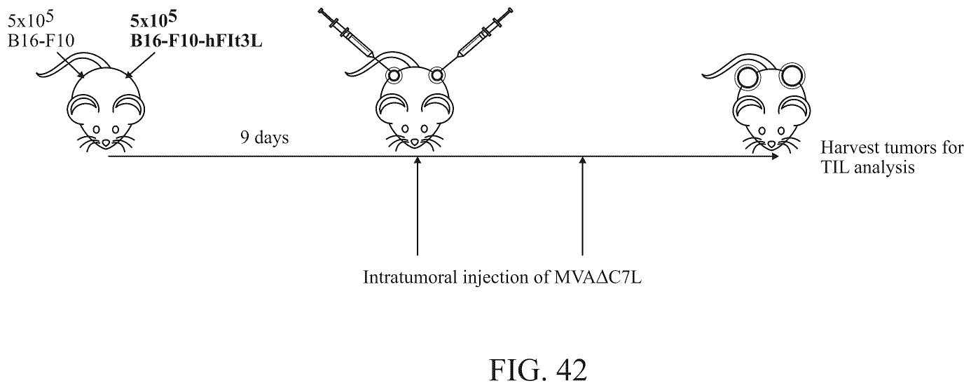

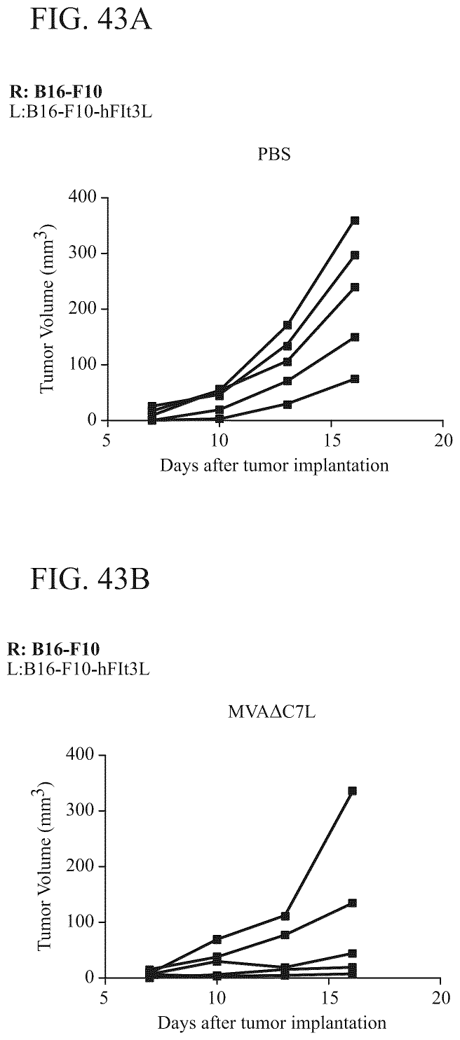

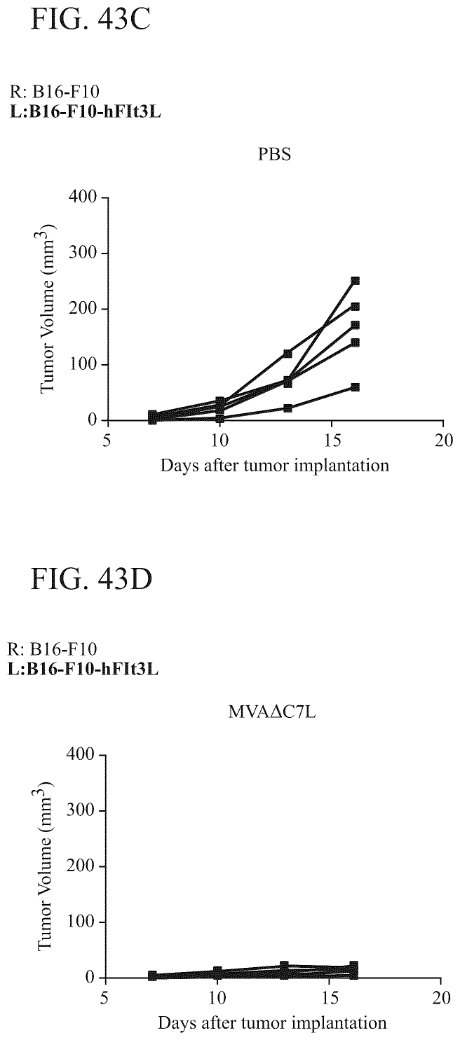

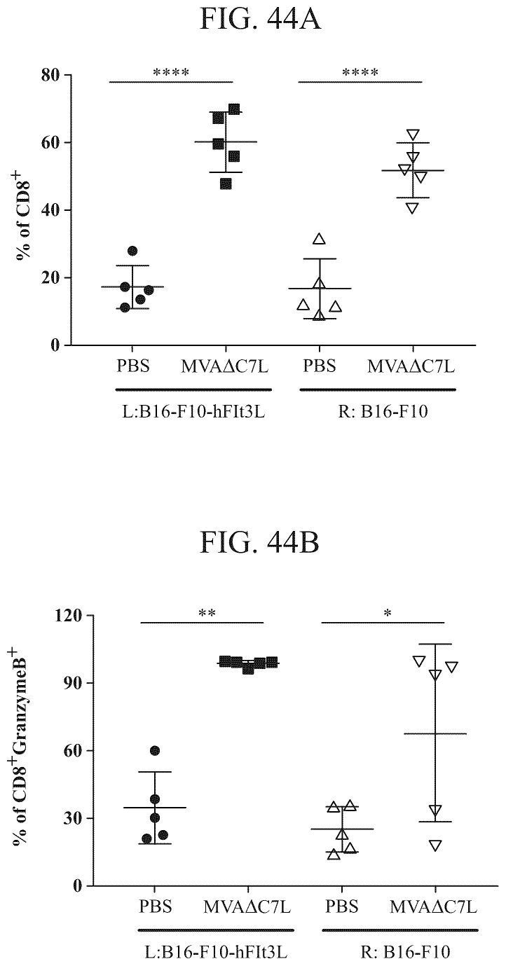

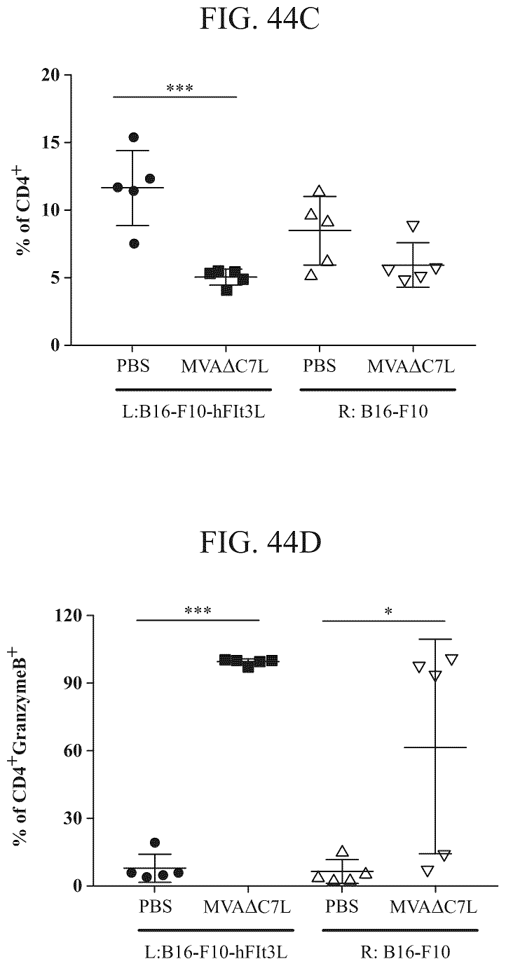

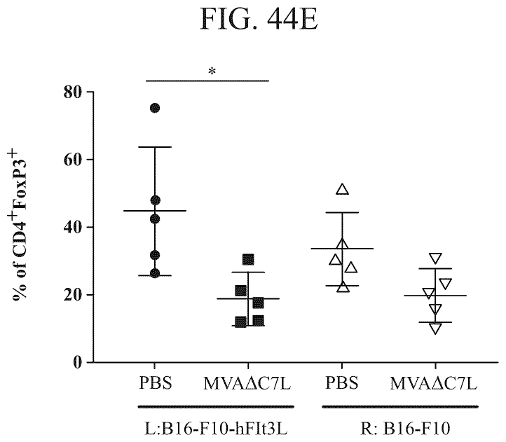

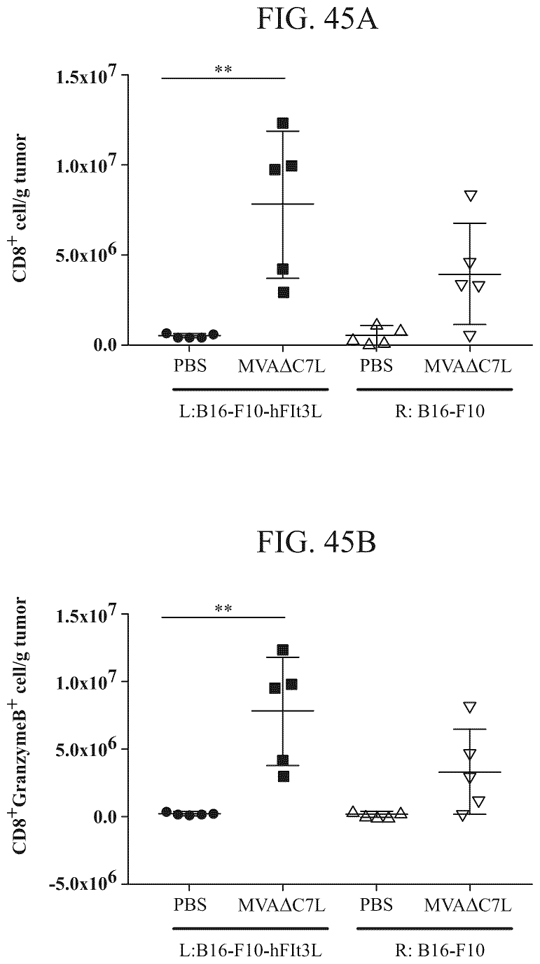

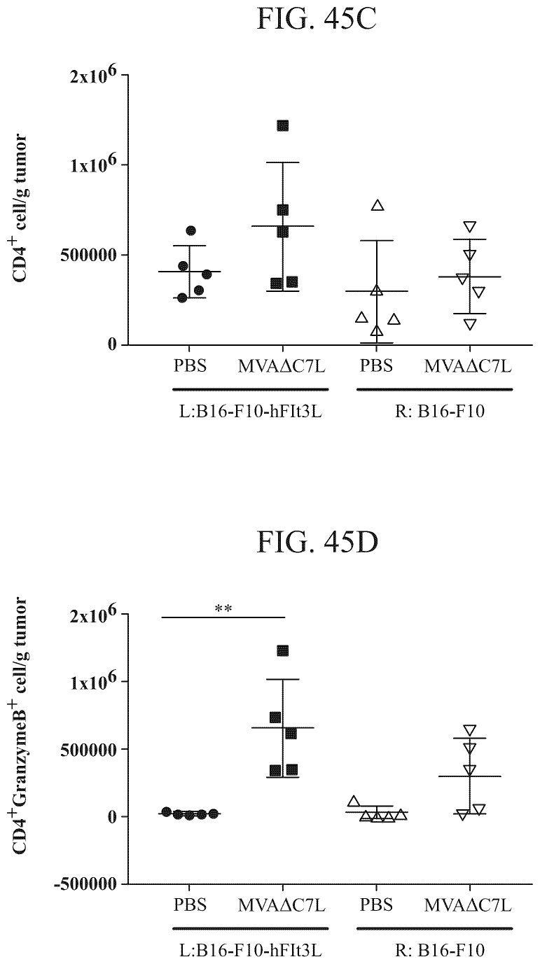

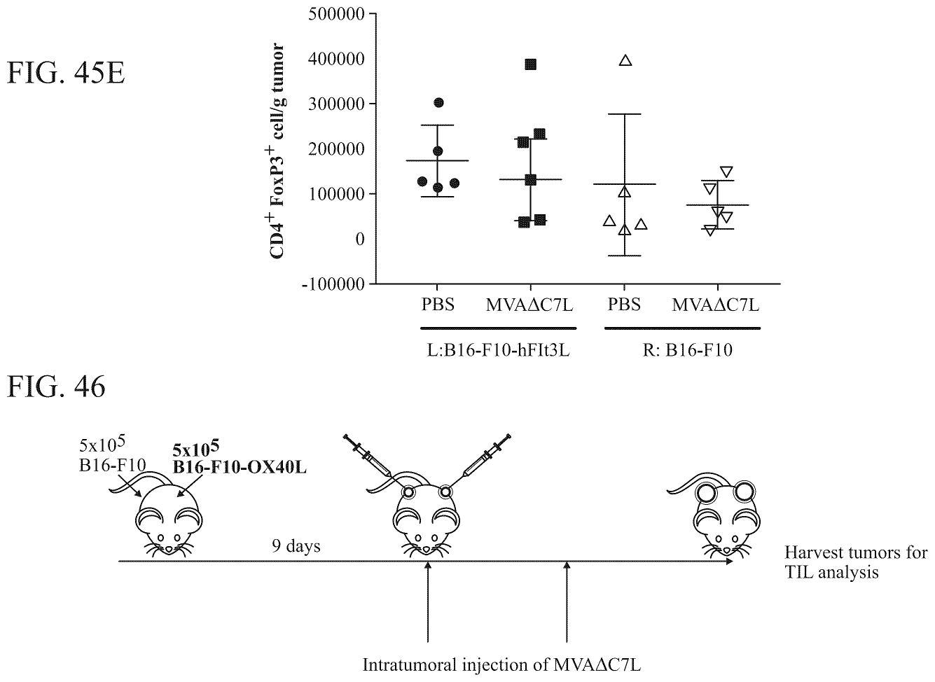

B16-F10 melanoma cells overexpressing hFlt3L (B16-F10-hFlt3L) are more responsive to intratumoral delivery of MVAΔC7L. Briefly, 5×10⁵ B16-F10 melanoma cells were implanted intradermally to right flanks of C57B/6J mice and 5×10⁵ B16-F10-hFlt3L melanoma cells were implanted intradermally to left flanks of C57B/6J mice. Nine days post tumor implantation, PBS or 4×10⁷ pfu of MVAΔC7L were intratumorally injected twice weekly to the tumors on both flanks (FIG. 42). Tumor volumes from the right and left flanks of individual mice were measured (FIGS. 43A-43D). Tumors were harvested 2 days post second injection and tumor infiltrating lymphocytes were analyzed by FACS (FIGS. 44A-44E and 45A-45E). B16-F10 and B16-F10-hFlt3L tumors treated with PBS had similar growth rate. In mice treated with MVAΔC7L, B16-F10-hFlt3L tumor growth were significantly inhibited compared with B16-F10 tumors. Intratumoral injection of MVAΔC7L generated higher percentage of CD8⁺ T cells (FIGS. 44A and 45A) and reduced CD4⁺ T cells (FIGS. 44C and 45C) and CD4⁺FoxP3⁺ T cells (FIGS. 44E and 45E) in both B16-F10 and B16-F10-hFlt3L tumors compared with PBS group. IT MVAΔC7L induced more CD8⁺GranzymeB⁺ (FIGS. 44B and 45B) and CD4⁺GranzymeB⁺ T cells (FIGS. 44D and 45D) in B16-F10-hFlt3L tumors. These results demonstrate that B16-F10 melanoma cells overexpressing hFlt3L are more responsive to intratumoral delivery of MVAΔC7L with enhanced T cell activation.

### Example 52: B16-F10 Melanoma Cells Overexpressing mOX40L are More Responsive to Intratumoral Delivery of MVAΔC7L

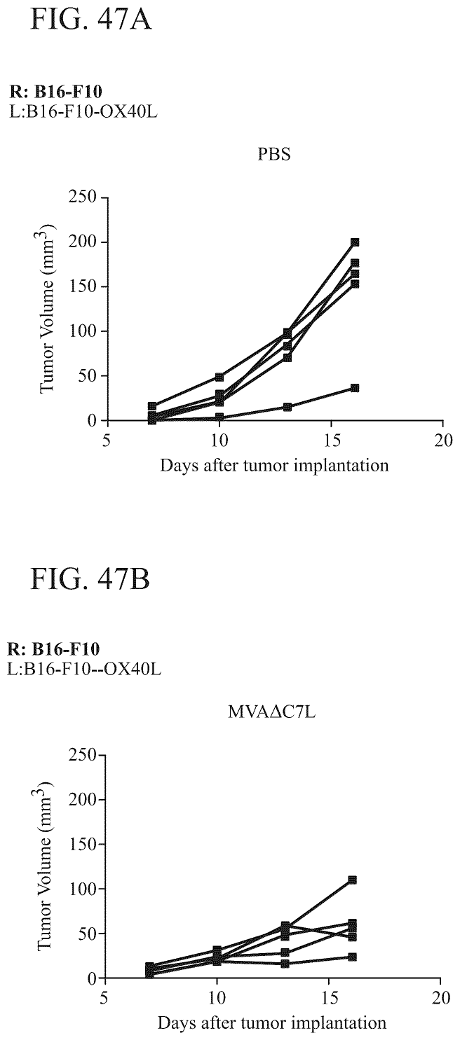

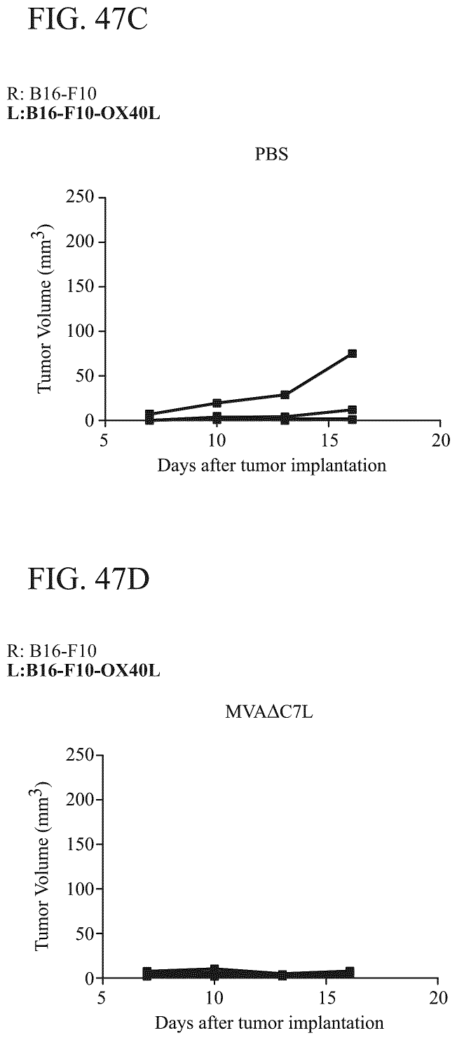

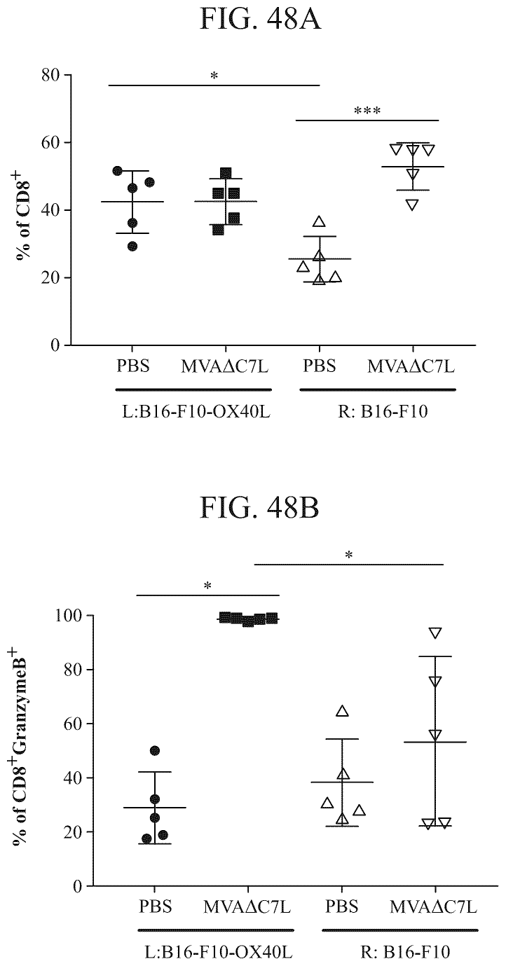

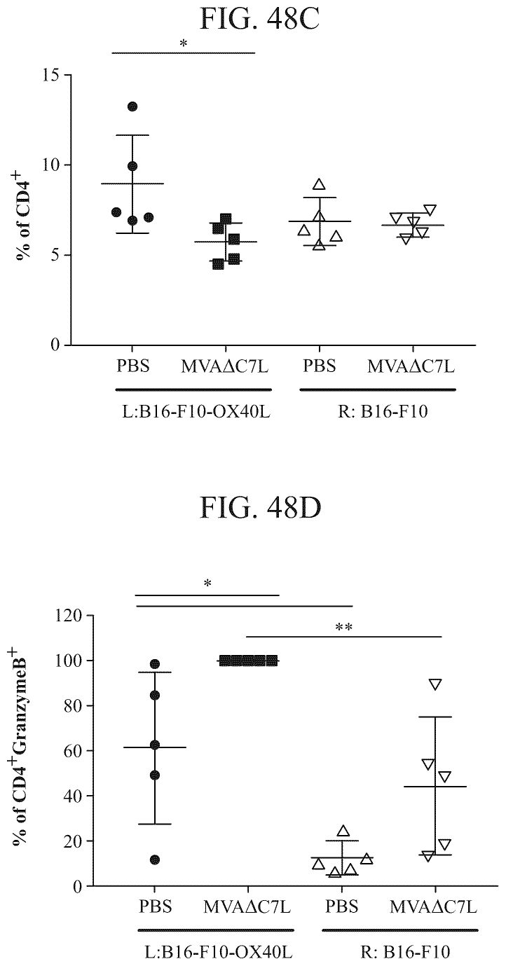

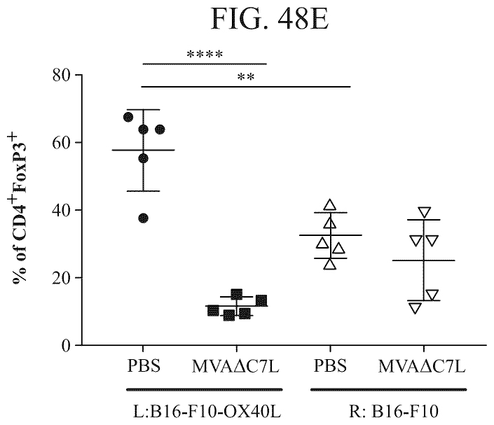

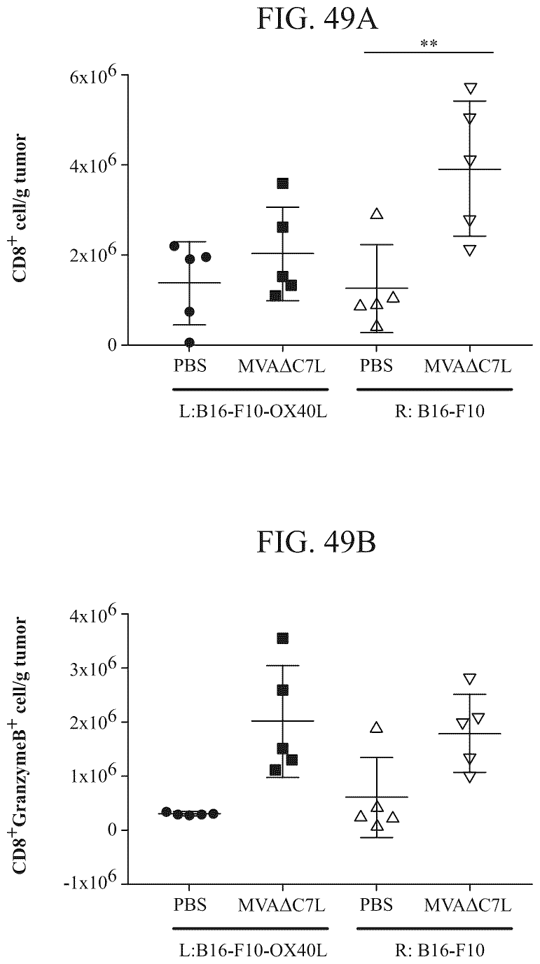

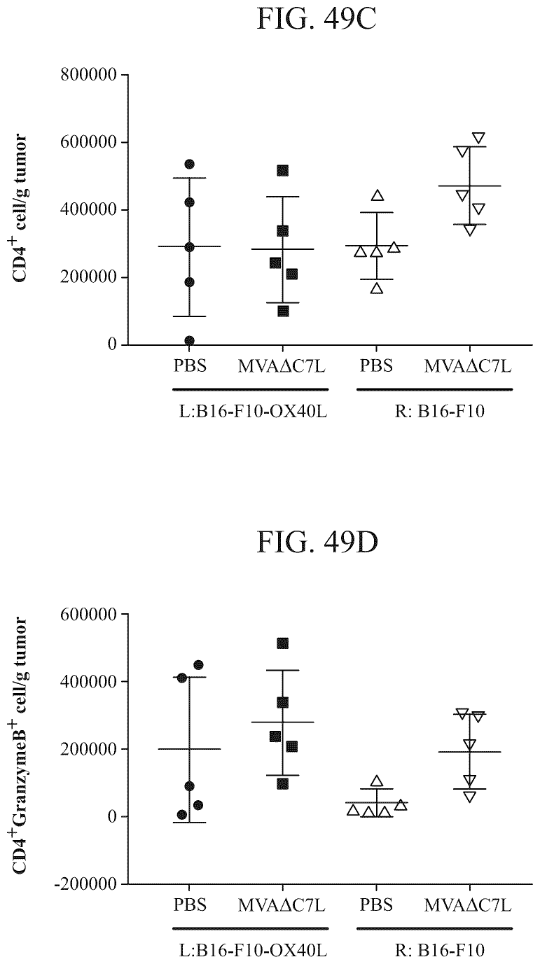

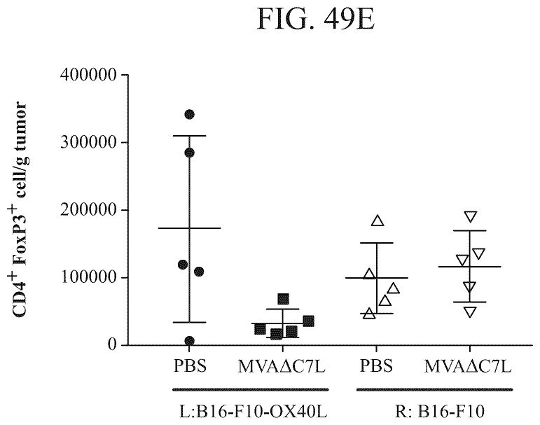

B16-F10 melanoma cells overexpressing mOX40L (B16-F10-OX40L) are more responsive to intratumoral delivery of MVAΔC7L. Briefly, 5×10⁵ B16-F10 melanoma cells were implanted intradermally to right flanks of C57B/6J mice and 5×10⁵ B16-F10-OX40L melanoma cells were implanted intradermally to left flanks of C57B/6J mice. Nine days post tumor implantation, PBS or 4×10⁷ pfu of MVAΔC7L were intratumorally injected twice weekly to the tumors on both flanks (FIG. 46). Tumor volumes from the right and left flanks of individual mice were measured twice weekly (FIGS. 47A-47D). Tumors were harvested 2 days post second injection and tumor infiltrating lymphocytes were analyzed by FACS (FIGS. 48A-48E and 49A-49E). B16-F10-OX40L tumors grew slower than B16-F10 tumors even in PBS treated group (FIGS. 47A and 47C). In mice treated with MVAΔC7L intratumorally, B16-F10 tumor growth was delayed (FIG. 47B) and B16-F10-OX40L tumor growth was significantly suppressed (FIG. 47D). Intratumoral injection of MVAΔC7L generated more activated CD8⁺ (FIGS. 48A and 49A) and CD4⁺ T cells (FIGS. 48C and 49C) in B16-F10 tumors compared with PBS treated group. Remarkably, 99%˜100% CD8⁺ and CD4⁺ T cells infiltrating B16-F10-OX40L tumors were GranzymeB⁺ (FIGS. 48B and 48D; 49B and 49D). More CD4⁺FoxP3⁺ T cells were infiltrating B16-F10-OX40L tumors than B16-F10 tumors but IT MVAΔC7L depleted these cells efficiently from an average of 60% to 15% out of total CD4⁺ T cells (FIGS. 48E and 49E). These results demonstrate that B16-F10 melanoma cells overexpressing OX40L are more responsive to intratumoral delivery of MVAΔC7L. OX40L plays an important role in activation of CD8⁺ and CD4⁺ T cell. IT MVAΔC7L is able to deplete CD4⁺FoxP3⁺ T cells in tumor microenvironment.

### Example 53: FTY720 Treatment Enhances the Anti-Tumor Efficacy of IT MVAΔC7L-hFlt3L-TK(−)-mOX40L with Delayed Tumor Growth and Prolonged Survival in B16-F10 Melanoma Unilateral Implantation Model

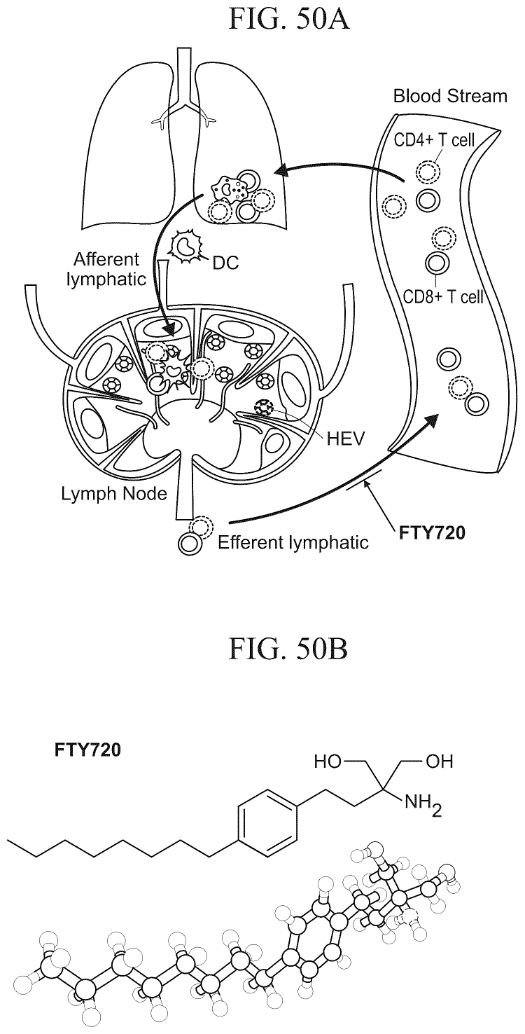

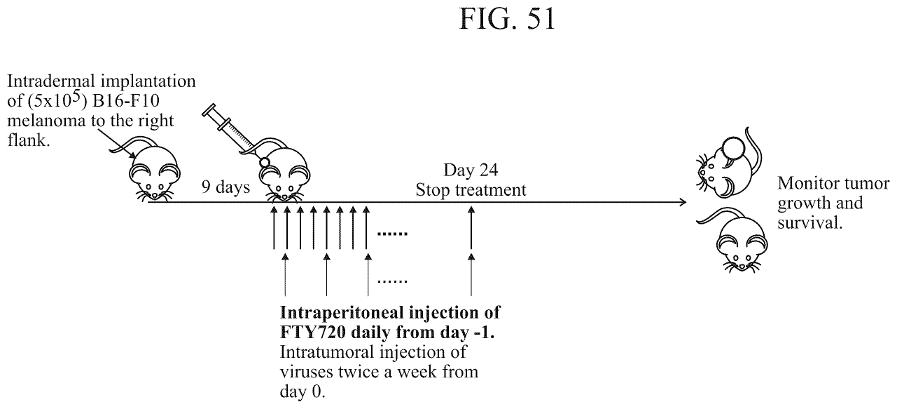

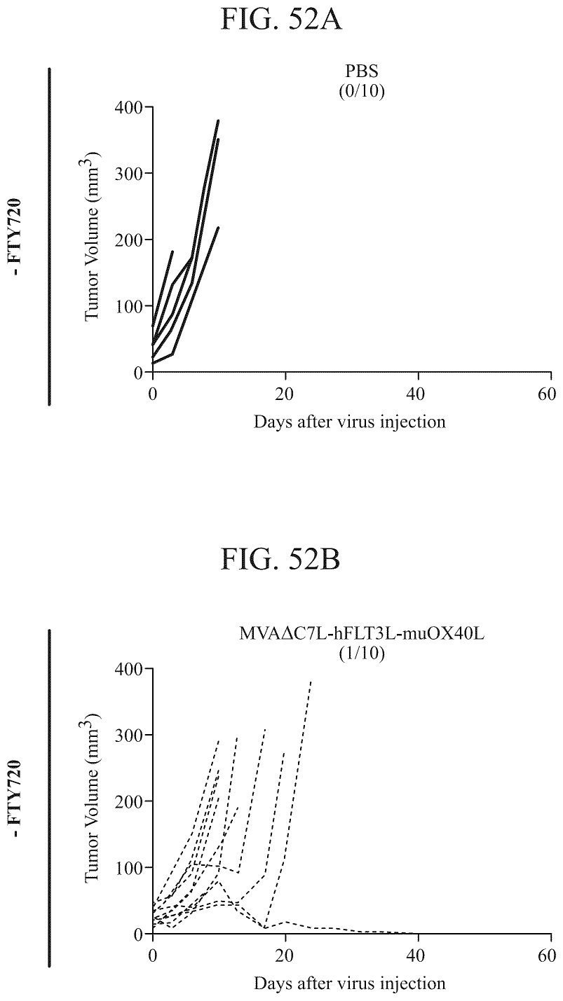

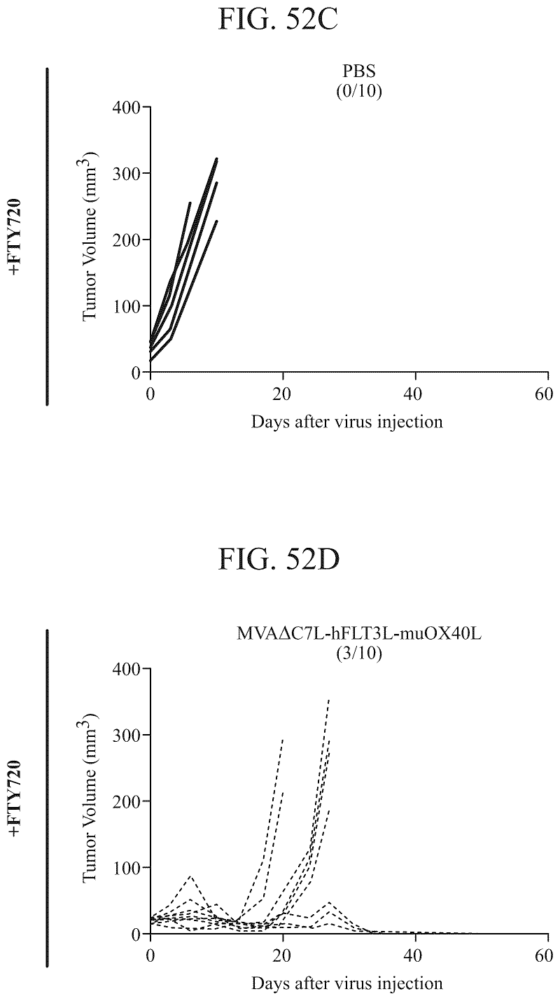

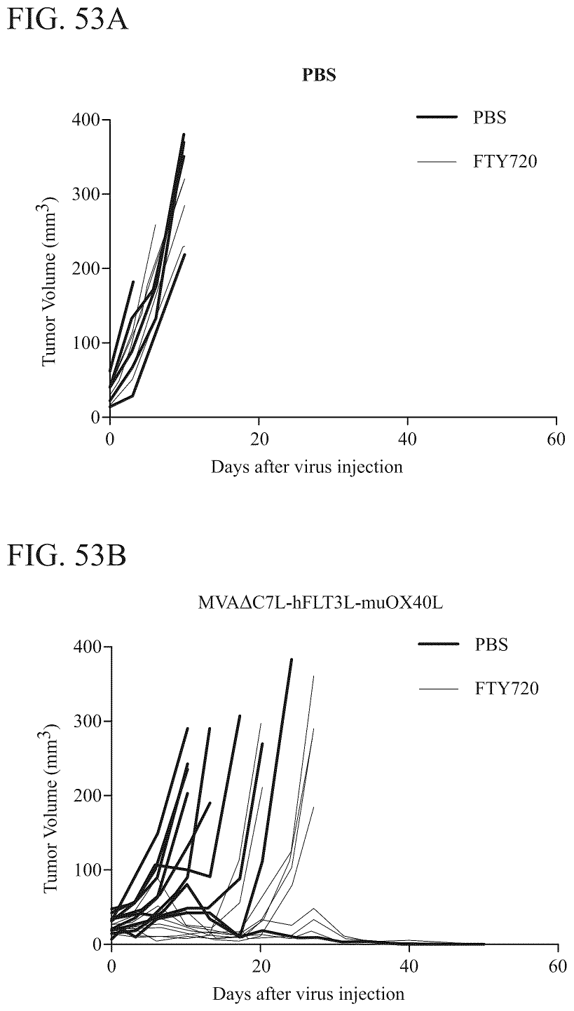

To determine whether lymph node T cell priming and activation is crucial for tumor eradication in IT MVAΔC7L-hFlt3L-TK(−)-mOX40L treatment, FTY720 (FIG. 50B) was used to block T cell exiting from lymphoid organs in a B16-F10 melanoma implantation model. Briefly, 5×10⁵ B16-F10 melanoma cells were implanted intradermally to right flanks of C57B/6J. Nine days post tumor implantation, PBS or 4×10⁷ pfu of MVAΔC7L-hFlt3L-TK(−)-muOX40L were intratumorally injected twice. FTY720 at 25 μg per mouse in 50% ethanol was given intraperitoneally daily during the treatment, beginning one day prior to the first MVAΔC7L-hFlt3L-TK(−)-muOX40L injection (FIG. 51). Tumor sizes were measured (FIGS. 52A-D; 53A and 53B) and survival was monitored (FIGS. 53C and 53D). FIG. 50A is a diagram demonstrating the mechanism of immune modulatory function of FTY720. After FTY720 treatment, T cells are trapped in lymph nodes. In mice treated with PBS, tumors grew rapidly and were not affected by FTY720 (FIGS. 52A and 52C). IT MVAΔC7L-hFlt3L-TK(−)-muOX40L alone delayed tumor growth (FIG. 52B). FTY720 enhanced the anti-tumor effect of IT MVAΔC7L-hFlt3L-TK(−)-muOX40L with delayed tumor growth (FIG. 52D) and improved survival (FIG. 53D). These results demonstrate that in IT MVAΔC7L-hFlt3L-TK(−)-muOX40L treatment, T cells accumulated within tumors during tumor development play a crucial role in tumor eradication.

### Example 54: Vaccinia E5 is Highly Conserved within Poxvirus Family

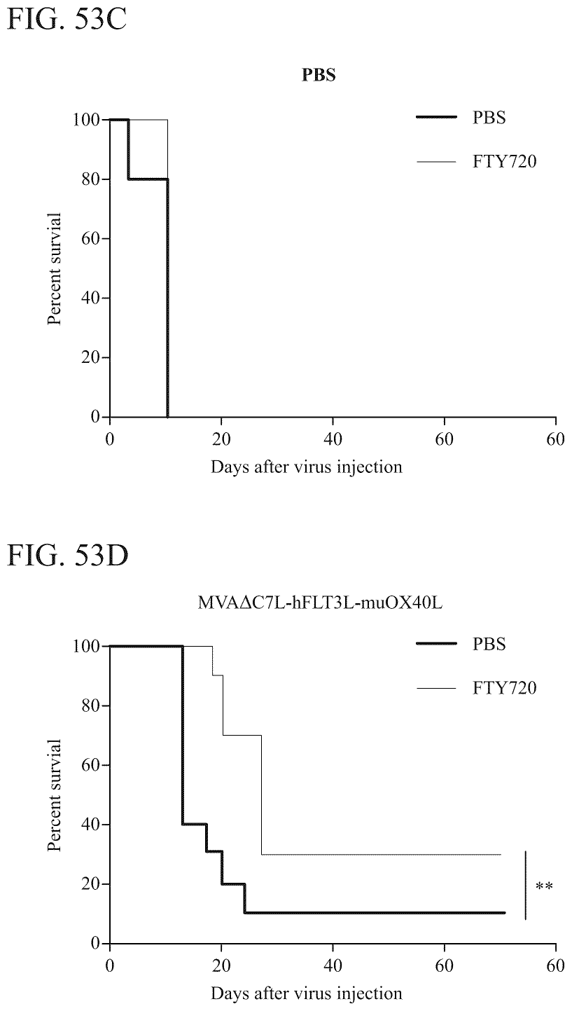

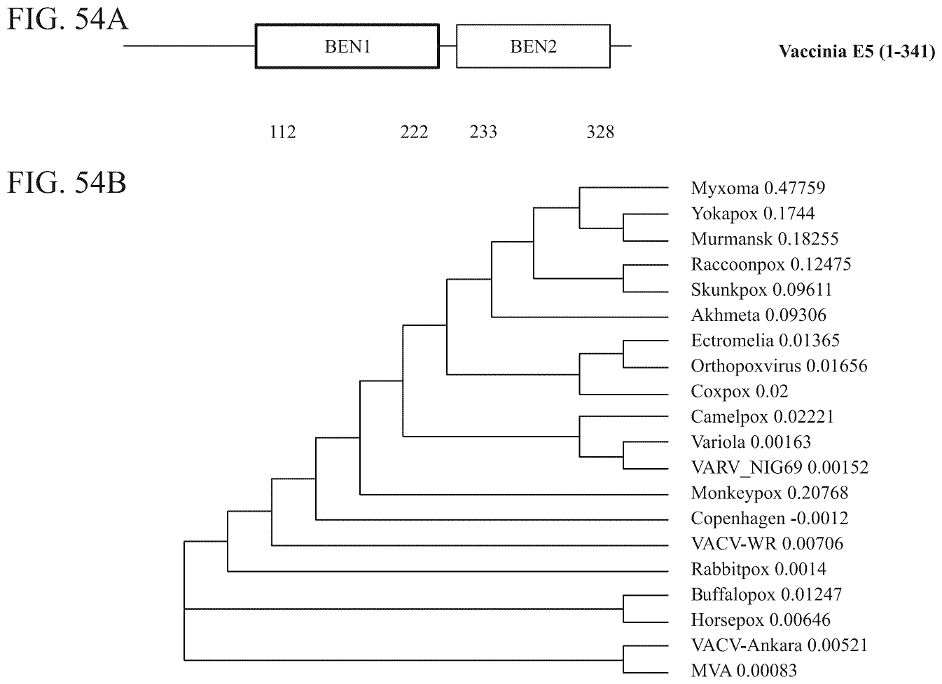

Vaccinia E5 is a 341-amino acid polypeptide, comprising two BEN domains at the C-terminus, from aa 112-222 and aa 233-328 (FIG. 54A). BEN is named after its presence in BANP/SMAR1, poxvirus E5R, and NACl. BEN domain containing proteins are involved in chromatin organization, transcription regulation, and possibly viral DNA organization. E5 is highly conserved among poxvirus family members. The E5 ortholog of myxoma virus, which belongs to the leporipox genus is the most divergent among all of the other poxvirus family members, including variola virus, which causes smallpox in humans, ectromelia (mouse pox), cowpox, and monkeypox (FIG. 54B).

### Example 55: Vaccinia E5 is a Virulence Factor

To test whether vaccinia E5 is a virulence factor, a recombinant VACVΔE5R virus was generated through homologous recombination at the flanking genes E4L and E6R. The E5R gene was replaced by the gene encoding mCherry under the control of a p7.5 promoter (FIG. 55A). An intranasal infection experiment with wild type vaccinia (WT VACV) at 2×10⁶ pfu and vaccinia virus with deletion of E5 (VACVΔE5R) at either 2×10⁷ pfu or 2×10⁶ pfu was performed using 6-8 week-old C57BL/6J female mice. All of the mice infected with WT VACV lost weight quickly starting the second day of infection. All of the mice either died or were euthanized due to more than 30% weight loss at day 7 to 8 post infection (FIGS. 55B and 55C). By contrast, mice infected with either 2×10⁶ pfu or 2×10⁷ pfu of VACVΔE5R lost close to 15% or 20% of initial body weight on average, respectively, at day 5 and 6 post infection, and then gained weight, and recovered from the infection (FIGS. 55B and 55C). These results indicate that VACVΔE5R is highly attenuated compared with WT VACV and E5 is a virulence factor.

### Example 56: VACVΔE5R Induces Higher Levels of IFNB Gene Expression and IFN-□ Protein Secretion from Bone Marrow-Derived Dendritic Cells (BMDCs) Compared with MVA

WT VACV infection of BMDCs fails to induce type I IFN, whereas MVA infection does (Dai et al. PLoS Pathogens (2014)). It was examined whether deletion of E5R from VACV gained the ability to induce IFNB in infected BMDCs. Bone marrow cells from C57BL/6J were cultured in the presence of GM-CSF. BMDCs were infected with either MVA, VACV, or VACVΔE5R at a MOI of 10. Cells were collected at 6 h post infection.

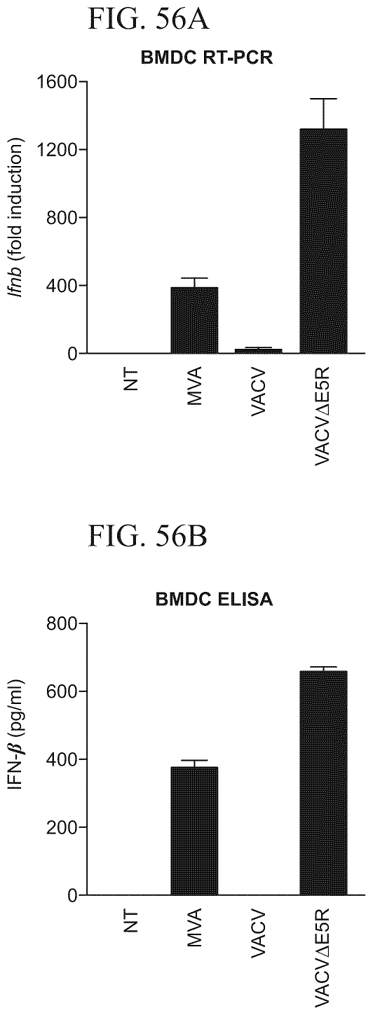

RNAs were extracted and RT-PCRs were performed. Supernatants were collected at 21 h post infection and IFN-□□ levels were measured by ELISA. RT-PCR results demonstrated that WT VACV infection of BMDCs induced a 29-fold IFNB gene expression compared with no-treatment control (NT), MVA infection induced 387-fold and VACVΔE5R induced 1316-fold (FIG. 56A). ELISA results demonstrated that VACV infection of BMDCs failed to induce IFN-□ secretion from BMDCs. However, IFN-□ levels in the supernatants of BMDCs infected with either MVA or VACVΔE5R were 375.5 μg/ml and 660 μg/ml, respectively (FIG. 56B). These results indicate that VACVΔE5R induces higher levels of IFNB gene expression and IFN-□ protein secretion from BMDCs compared with MVA.

### Example 57: MVAΔE5R Induces Higher Levels of IFNB Gene Expression in BMDCs and BMDMs Compared with MVA

To test whether deletion of E5 from MVA genome also enhances its ability to induce IFNB gene induction, a recombinant MVAΔE5R was generated through homologous recombination at the flanking genes E4L and E6R, which resulted in the replacement of the E5R gene by the gene encoding mCherry under the control of a p7.5 promoter (FIG. 57A). A recombinant MVAΔK7R was also generated through homologous recombination at the flanking genes K5,6L and F1L, which also resulted in the replacement of the K7R gene by the gene encoding mCherry under the control of a p7.5 promoter (FIG. 57B).

BMDCs or BMDMs were generated by culturing bone marrow cells in the presence of GM-CSF or M-CSF, respectively. BMDCs and BMDMs were infected with either MVA, MVAΔE5R, or MVAΔK7R at a MOI of 10. Cells were collected at 6 h post infection. RT-PCR demonstrated that MVAΔE5R, MVAΔK7R, or MVA infection of BMDCs resulted in 2452-fold, 22-fold, or 12-fold induction of IFNB gene, respectively, compared with a “Blank” control (FIG. 57C). In BMDMs, MVAΔE5R, MVAΔK7R, or MVA infection resulted in 4510-fold, 35-fold, or 4-fold induction of IFNB gene expression compared with a “Blank” control (FIG. 57D). These results demonstrate that E5 is a dominant inhibitor of IFNB gene induction in BMDCs and BMDMs, and deletion of E5 from the MVA genome leads to a dramatic induction of IFNB. These results also demonstrate that K7 is also an inhibitor of IFNB gene induction in BMDCs and BMDMs.

### Example 58: MVAΔE5R Induces Higher Levels of IFNA, CCL4, and CCL5 Gene Expression in BMDCs Compared with MVA

BMDCs were infected with either MVA or MVAΔE5R at a MOI of 10. Cells were collected at 6 h post infection. RT-PCR analyses demonstrated that MVAΔE5R also induced much higher levels of IFNA (FIG. 58A), CCL4 (FIG. 58B), and CCL5 (FIG. 58C) gene expression compared with MVA. These results suggest that MVAΔE5R infection triggers the induction of type I IFN and chemokines that may facilitate the recruitment immune cells into the site of infection.

### Example 59: MVAΔE5R Induces Higher Levels of IFNB Gene Expression Compared with Heat-Inactivated MVAΔE5R (Heat-iMVAΔE5R)

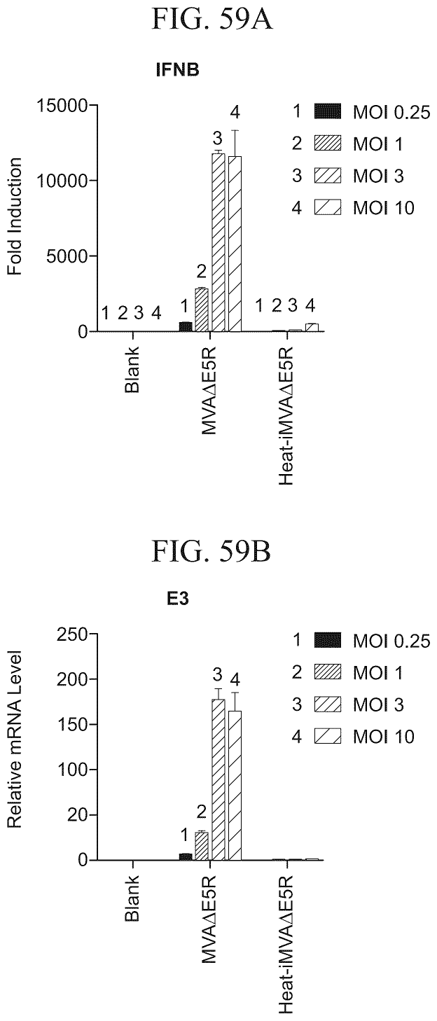

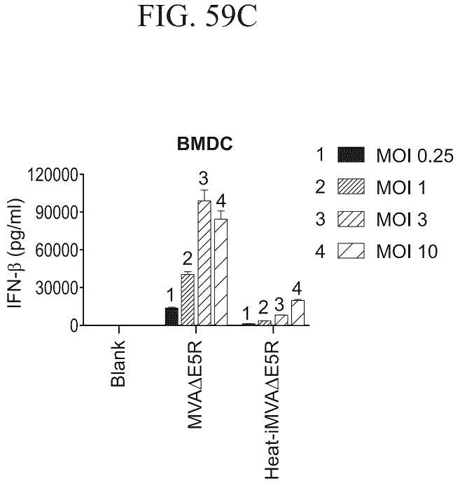

Heat-inactivated MVA induces higher levels of type I IFN and proinflammatory cytokines and chemokines compared with live MVA (Dai et al. Science Immunology (2017)). The induction of IFNB gene expression and IFN-□ secretion by MVAΔE5R-vs. Heat-iMVAΔE5R-infected BMDCs was examined. Briefly, BMDCs were infected with either MVAΔE5R or Heat-iMVAΔE5R at MOIs of 0.25, 1, 3, or 10. Cells were washed after 1 h infection and fresh medium was added. Cells and supernatants were collected at 14 h post infection. IFNB and E3 gene expressions were determined by RT-PCR. IFN-□ protein levels in the supernatants were determined by ELISA. RT-PCR results show that MVAΔE5R induces IFNB gene expression and IFN-□ secretion in a dose-dependent manner, and the induction was much higher compared with Heat-iMVAΔE5R. At MOIs of 3 or 10, MVAΔE5R resulted in the maximum levels of induction of IFNB gene expression and IFN-□ protein secretion (FIGS. 59A and 59C). MVAΔE5R expressed vaccinia E3 gene, whereas Heat-inactivated MVAΔE5R failed to express as expected (FIG. 59B).

### Example 60: MVAΔE5R-Induced IFNA and IFNB Gene Expression and IFN-b Protein Secretion from BMDCs Require the Cytosolic DNA Sensor cGAS

MVAΔE5R infection of BMDCs induced high levels of IFNA and IFNB gene induction and IFN-□ protein secretion compared with live MVA, Heat-inactivated MVA, or Heat-inactivated MVAΔE5R. To determine whether cGAS is required for MVAΔE5R-induced IFN gene expression and protein secretion, BMDCs from WT and cGAS^{-/-} mice were generated, and were infected with either MVA or MVAΔE5R at a MOI of 10. Cells were collected at 6 h post infection. RT-PCR analysis showed that MVAΔE5R-induced IFNB (FIG. 60A) and IFNA (FIG. 60B) gene expression is dependent on cGAS, whereas both MVA and MVAΔE5R express E3L gene in either WT or cGAS^{-/-} cells (FIG. 60C). BMDCs from WT and cGAS^{-/-} mice were infected with either MVA, MVAΔE5R, Heat-iMVA, or Heat-iMVAΔE5R at a MOI of 10, and supernatants were collected at 8 and 16 h post infection. IFN-□ protein levels in the supernatants were measured by ELISA. MVAΔE5R-induced higher levels of IFN-□ secretion from WT BMDCs compared with MVA, Heat-iMVA, or Heat-iMVAΔE5R at 8 h post infection. At 16 h post infection, the IFN-□ level of the supernatant from MVAΔE5R-infected WT BMDCs rose even higher compared with that in the supernatant collected at 8 h post infection (FIG. 60D). The induction of IFN-□ secretion by MVAΔE5R-infected BMDCs was completely dependent on cGAS (FIG. 60D).

### Example 61: MVAΔE5R-Induced IFNB Gene Expression and IFN-b Protein Secretion from BMDCs and BMDMS Require STING

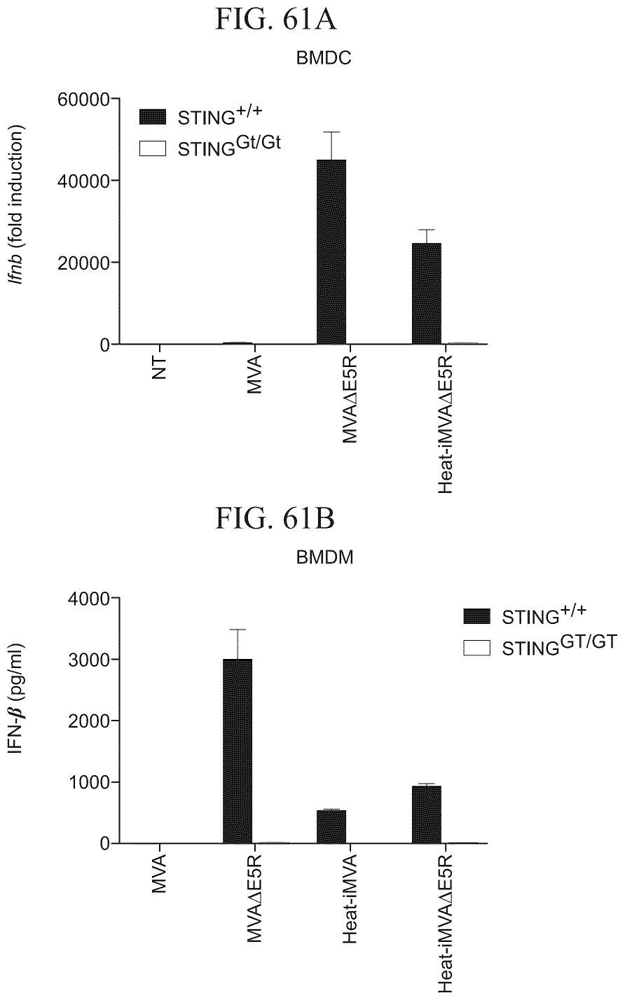

STING is an endoplasmic reticulum (ER)-localized protein critical for the cytosolic DNA-sensing pathway. Upon DNA-binding, cGAS is activated and generates a second messenger cyclic GMP-AMP (cGAMP) from ATP and GTP. cGAMP binds to STING and subsequently activates STING, which leads to activation of transcription factor IRF3, and IFNB gene induction. To test whether MVAΔE5R-induced IFNB gene expression and IFN-D protein secretion requires STING, BMDCs and BMDMs from age-matched WT and STING^{Gt/Gt} mice were generated, which lack functional STING protein. MVAΔE5R and Heat-iMVAΔE5R induced IFNB gene expression in WT BMDCs, but not in STINGG^{Gt/Gt} cells (FIG. 61A). Similarly, MVAΔE5R induced IFN-□□ protein secretion in WT BMDMs, but not in STING^{Gt/Gt} cells (FIG. 61B).

### Example 62: MVAΔE5R-Induced IFN-□ Protein Secretion from BMDCs Require IRF3, IRF7 and IFNAR1

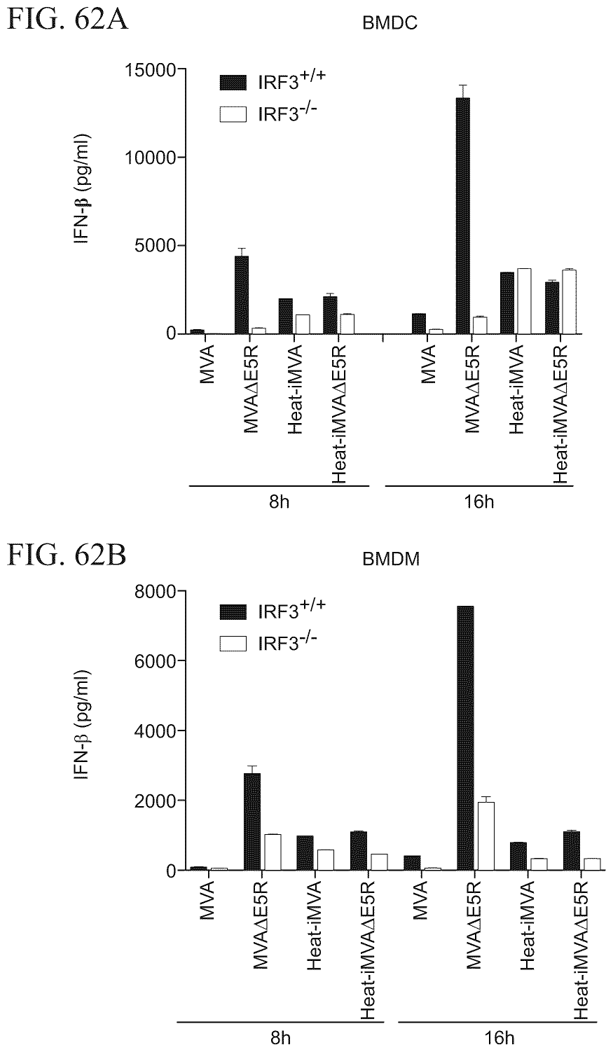

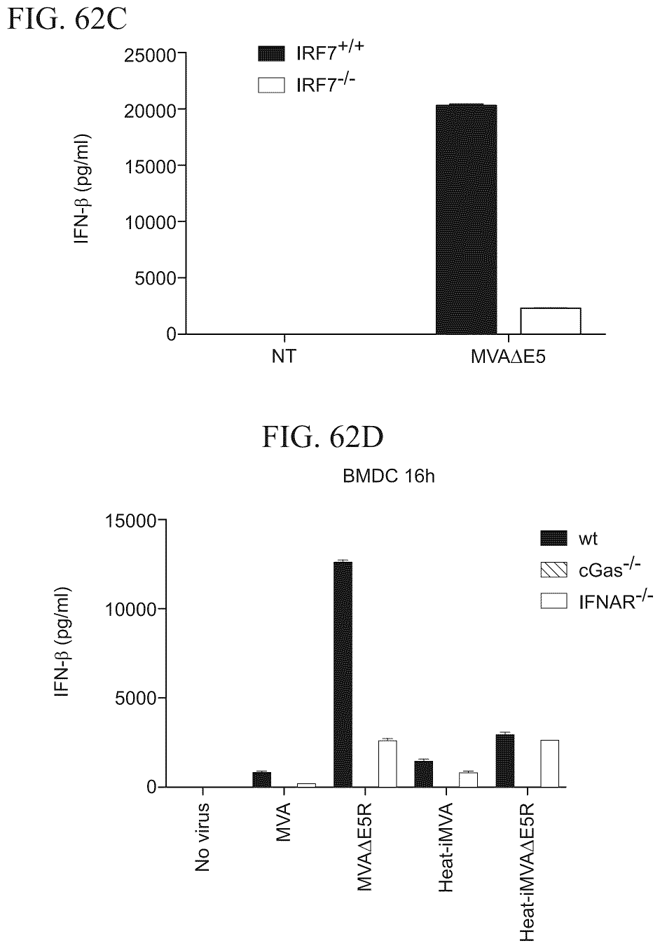

Transcription factors IRF3 and IRF7 are important for the induction of IFNB gene expression. Type I IFNs, once secreted, bind to IFNAR, which leads to the activation of JAK/STAT pathway and the induction of IFN-stimulated genes (ISGs). To determine whether IRF3, IRF7, and IFNAR1 were required for the induction of IFN-□ protein secretion from BMDCs and BMDMs, BMDCs and BMDMs from WT and IRF3^{-/-} mice were infected with either MVA, MVAΔE5R, Heat-iMVA, or Heat-iMVAΔE5R. Supernatants were collected at 8 and 16 h post infection. The IFN-□ protein levels in the supernatants were determined by ELISA. MVAΔE5R-induced IFN-□ secretion at both 8 and 16 h was reduced by 96% and 94% in IRF3^{-/-} BMDCs, respectively (FIG. 62A). In addition, MVAΔE5R-induced IFN-□ secretion at both 8 and 16 h was reduced by 63% and 75% in IRF3^{-/-} BMDCs, respectively (FIG. 62B).

BMDCs from WT and IRF7^{-/-} mice were infected with MVAΔE5R at a MOI of 10 or treated with mock control. Supernatants were collected at 16 h post infection. The IFN-□ protein levels in the supernatants were determined by ELISA. MVAΔE5R-induced IFN-□ secretion was reduced by 89% in IRF7^{-/-} BMDCs (FIG. 62C).

BMDCs from WT, cGAS^{-/-}, or IFNAR1^{-/-} mice were infected with either MVA, MVAΔE5R, Heat-iMVA, or Heat-iMVAΔE5R. Supernatants were collected at 16 h post infection. The IFN-□ protein levels in the supernatants were determined by ELISA. MVAΔE5R-induced IFN-□ secretion was abolished in cGAS^{-/-} BMDCs and was reduced by 79% in IFNAR1^{-/-} BMDCs (FIG. 62D).

Taken together, these results demonstrate that IRF3/IRF7/IFNAR1 play important roles in the induction of IFN-□ production by BMDCs.

### Example 63: WT VACV-Induced cGAS Degradation is Mediated Through a Proteasome-Dependent Pathway

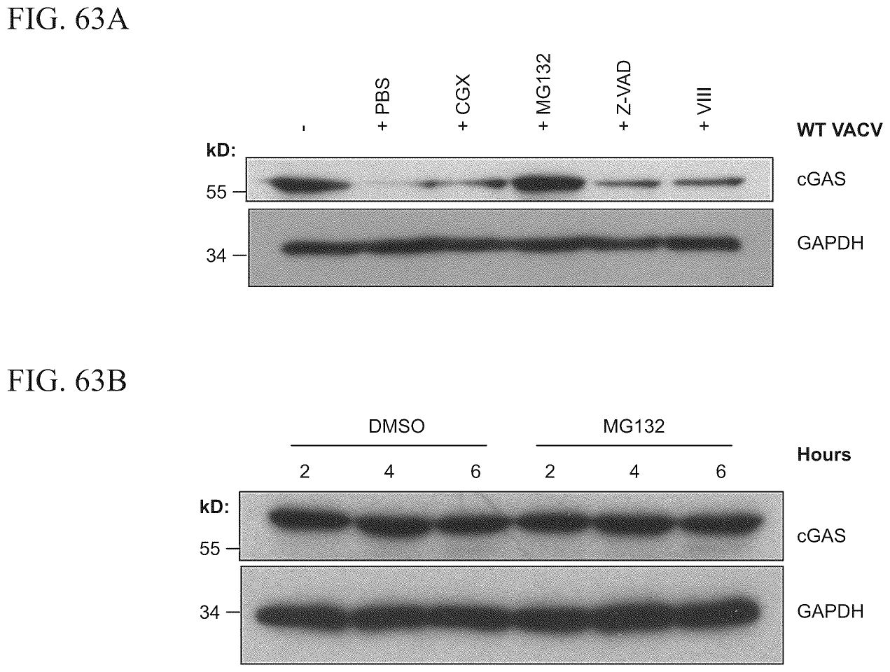

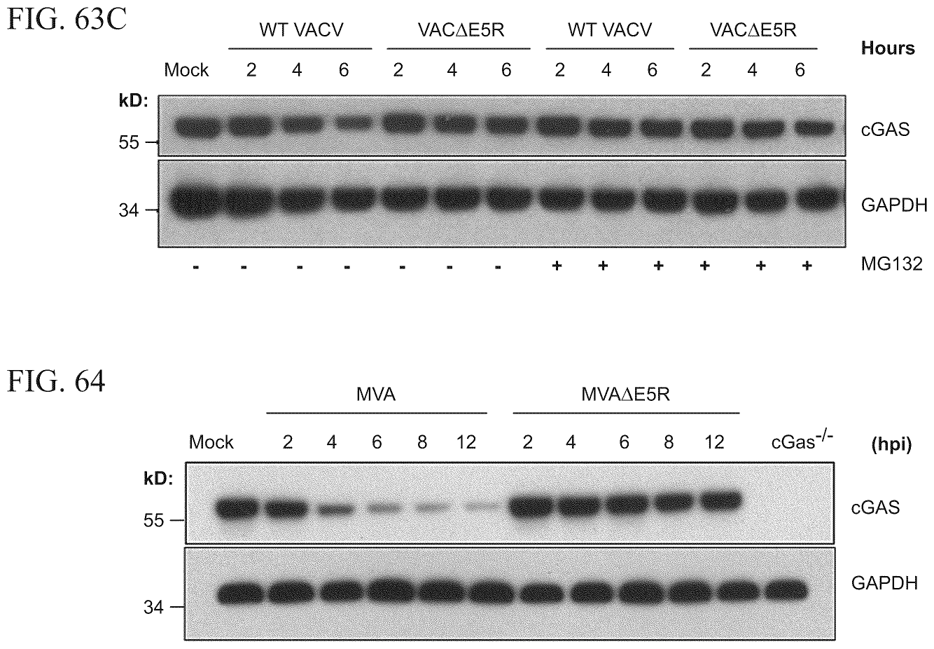

It has been determined that WT VACV infection triggers degradation of cGAS in murine embryonic fibroblasts (MEFs) and BMDCs. To determine the mechanism of VACV-induced cGAS degradation, MEFs were pre-treated with either cycloheximide (CHX); a proteasomal inhibitor, MG132; a pan-caspase inhibitor, Z-VAD; or an AKT1/2 inhibitor VIII for 30 min. MEFs were then infected with WT VACV in the presence of each drug. Cells were collected at 6 h post infection. Western blot analysis demonstrated that in the presence of MG132, WT VACV-induced cGAS degradation was blocked (FIG. 63A). As a control, treatment of MEFs with either DMSO or MG132 did not affect cGAS protein level (FIG. 63B). To test whether vaccinia E5 is responsible for WT VACV-mediated cGAS degradation, BMDCs were infected with either WT VACV or VACVΔE5R in the presence or absence of MG132 (FIG. 63C). Whereas WT VACV infection of BMDCs resulted in cGAS degradation, VACVΔE5R infection did not. In addition, WT VACV-induced cGAS degradation was blocked in the presence of MG132. These results further support that E5 of vaccinia virus is responsible for WT VACV-induced cGAS degradation.

### Example 64: The E5R Gene in MVA is Important in Mediating cGAS Degradation in BMDCs

To test whether the E5R gene in MVA has similar role to vaccinia E5R gene, BMDCs were infected with either MVA or MVAΔE5R at a MOI of 10. Cells were collected at 2, 4, 6, 8, and 12 h post infection. A cGAS^{-/-} BMDC sample without infection was also included.

Western blot analysis showed that infection of BMDCs with MVA also caused rapid degradation of cGAS, whereas infection with MVAΔE5R resulted in much less cGAS degradation (FIG. 64). These results demonstrate that the E5R gene in MVA is also responsible for cGAS degradation.

### Example 65: MVAΔE5R Induces Higher Levels of Phosphorylated Stat2 Compared with MVA

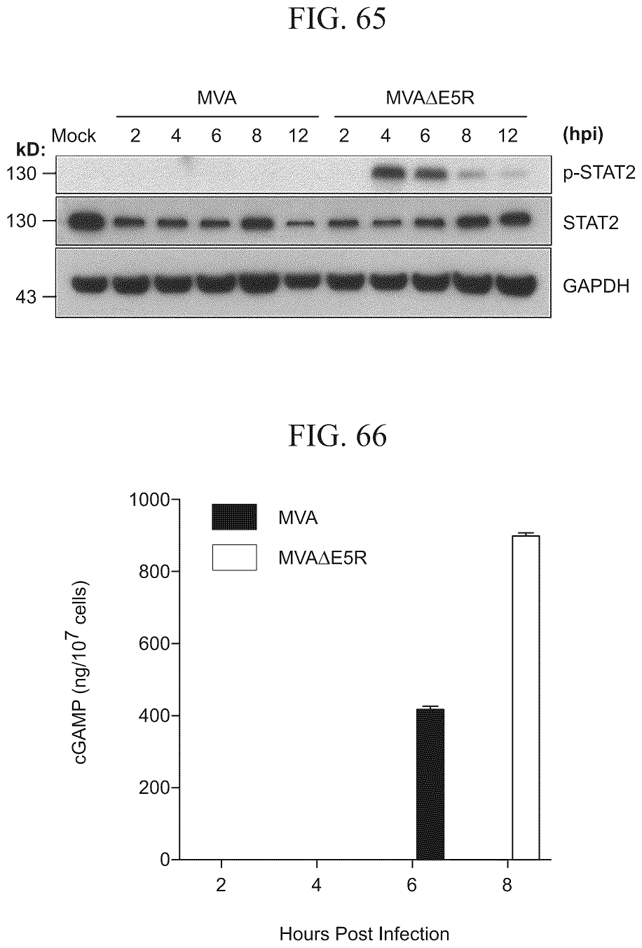

To test whether MVAΔE5R infection of BMDCs triggers a stronger IFNR downstream signaling, BMDCs were infected with either MVA or MVAΔE5R at a MOI of 10. Cells were collected at 2, 4, 6, 8, and 12 h post infection. Western blot analysis was performed using anti-phospho-STAT2, anti-STAT2, and anti-GAPDH antibodies (FIG. 65). The results demonstrate that MVAΔE5R induces higher levels of p-STAT2, especially at 4 and 6 h post infection, compared with MVA. STAT2 is an important transcription factor for the induction IFN-stimulated genes. Phosphorylated STAT2 translocates from the cytoplasm to the nucleus to bind to IFN-sensitive response element (ISRE), which leads to the induction of ISG expression. These results indicate that MVAΔE5R infection can trigger stronger induction of hundreds of ISGs through p-STAT2 compared with MVA. Some ISGs are important cytokines and chemokines, which are important for T cell activation and recruitment of other immune cells to the site of infection.

### Example 66: MVAΔE5R Induces High Levels of cGAMP Production in Infected BMDCs

Upon DNA binding, cGAS is activated, and converts ATP and GTP to cyclic GMP-AMP (cGAMP), which acts as a second messenger, resulting in the activation of the STING/TBK1/IRF3 axis and induction of type I IFN. To determine whether MVAΔE5R leads to higher levels of cGAMP production, 2.5×10⁶ BMDCs were infected with either MVA or MVAΔE5R at a MOI of 10. Cells were collected at 2, 4, 6 and 8 h post infection. cGAMP concentrations were measured by incubating cell lysates with permeabilized differentiated THP1-Dual™ cells, which were derived from the human THP-1 monocyte cell line by stable integration of two inducible reporter constructs (FIG. 66). Supernatants were collected at 24 h, and luciferase activities (as an indication for IRF pathway activation) were measured. cGAMP levels were calculated by comparing with cGAMP standards. MVAΔE5R induced close to 400 ng/10⁷ cells at 6 h post infection, and 900 ng/10⁷ cells at 8 h post infection. By contrast, MVA-induced cGAMP level was not detectable by this method, which is less sensitive than mass spectrometry. Given the increase of cGAMP levels produced during the first 8 h of MVAΔE5R infection, it is plausible that E5 protein not only prevents parental viral DNA recognition by cGAS, it also prevents detection of progeny viral DNA by cGAS. E5 has been shown to be in the virosomes/viral factories, where viral DNA replication occurs. These results indicate that E5 inhibits cGAMP production by cGAS.

### Example 67: MVAΔE5R Induces IFN-β Protein Secretion from Plasmacytoid Dendritic Cells (pDCs)

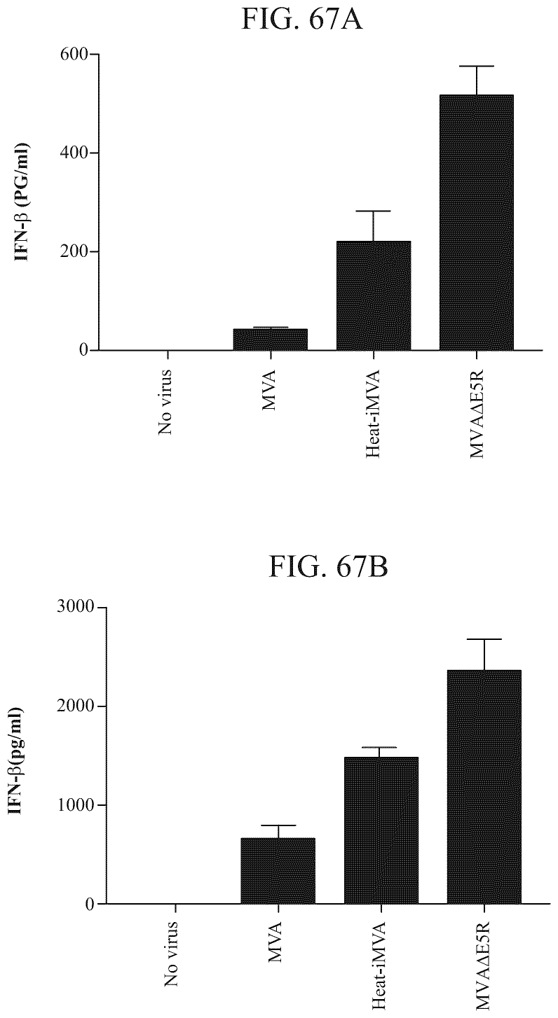

pDCs are potent type I IFN producing cells. To test whether MVAΔE5R infection of pDCs induces type I IFN production, 1.2×10⁵ pDCs (B220⁺PDCA-1⁺) sorted from splenocytes were infected with either MVA, Heat-iMVA, or MVAΔE5R. Non-infected splenocytes were included as a control. Supernatants were collected at 18 h post infection. IFN-β levels in the supernatants were measured by ELISA. MVAΔE5R induced higher levels of IFN-β protein secretion from splenic pDCs compared with MVA and Heat-iMVA (FIG. 67A); 517.5 μg/ml in the supernatants from MVAΔE5R-infected splenic pDCs vs. 43.5 μg/ml in the supernatants from MVA-infected splenic pDCs vs. 220 μg/ml in the supernatants of Heat-iMVA-infected splenic pDCs (FIG. 67A).

### Example 68: MVAΔE5R-Induced IFN-β Protein Secretion from pDCs is Dependent on cGAS

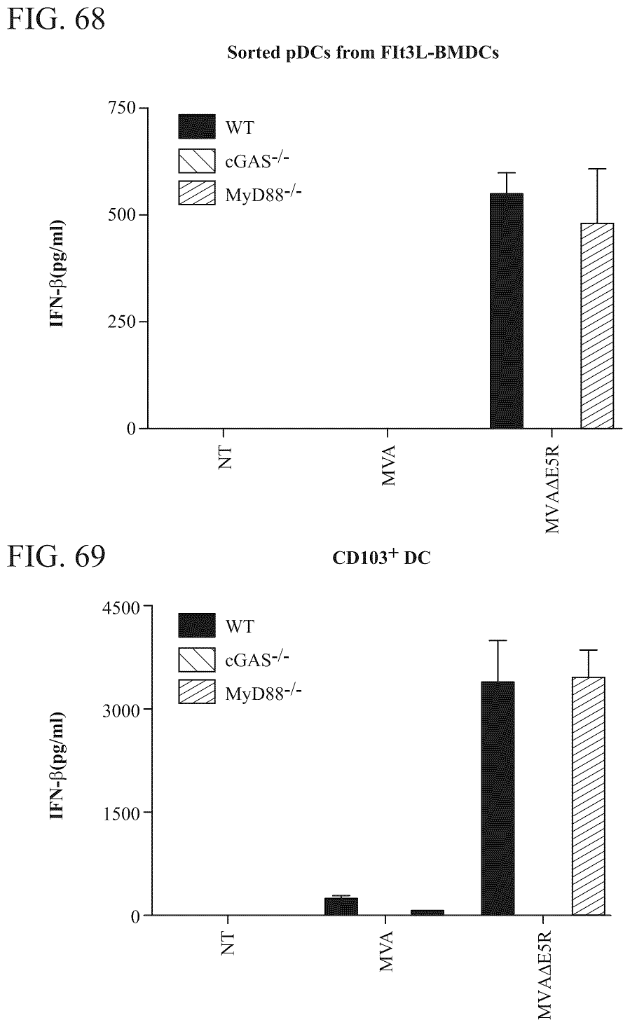

pDCs commonly use endosomal-localized TLR7 and TLR9 to detect endosomal RNA and DNA to elicit strong type I IFN responses. MyD88 is an adaptor for both TLR7 and TLR9. More recently, it has been shown that cGAS is also important for detecting cytosolic DNA in pDCs. To test whether cGAS or MyD88 pathway is important for MVAΔE5R-induced type I IFN production, pDCs were sorted from Flt3L-cultured BMDCs (B220⁺PDCA-1⁺) obtained from WT, cGAS^{-/-}, or MyD88^{-/-} mice. 2×10⁵ cells were infected with either MVA or MVAΔE5R. No treatment (NT) control was included. Supernatants were collected at 18 h post infection. IFN-β levels in the supernatants were measured by ELISA. The results show that MVAΔE5R-induced IFN-□ production was abolished in cGAS^{-/-} Flt3L-pDCs and was largely unchanged in MyD88^{-/-} Flt3L-pDCs (FIG. 68).

### Example 69: MVAΔE5R-Induced IFN-β Protein Secretion from CD103⁺ DCs is Dependent on cGAS

CD103⁺ DCs are a subset of conventional DCs important for cross-presenting antigens. Transcription factor Batf3 is important for the development of CD103⁺ DCs. CD103⁺ DCs are critical for cross-presenting tumor antigens and initiate antitumor immunity. To test whether MVAΔE5R induces IFN-□ protein secretion from sorted Flt3L-CD103⁺ DCs and whether cGAS or MyD88 is important for the induction, CD103⁺ DCs were sorted from Flt3L-cultured BMDCs (CD11c⁺CD103⁺) obtained from WT, cGAS^{-/-}, or MyD88^{-/-} mice. 2×10⁵ cells were infected with either MVA or MVAΔE5R. NT control was included. Supernatants were collected at 18 h post infection. IFN-β levels in the supernatants were measured by ELISA. The results show that MVAΔE5R potently induce IFN-□ protein secretion from sorted Flt3L-CD103⁺ DCs. The IFN-Q concentration in the supernatants from MVAΔE5R-infected CD103⁺ DCs was 3610 μg/ml, whereas the IFN-□ concentration in the supernatants from MVA-infected CD103⁺ DCs was 365.5 μg/ml (FIG. 69). Furthermore, the induction of IFN-□ protein secretion from sorted Flt3L-CD103⁺ DCs by MVAΔE5R is completely dependent on cGAS and MyD88 is not required for the induction effect (FIG. 69).

### Example 70: MVAΔE5R Infection of BMDCs Results in Lower Levels of Cell Death Compared with MVA

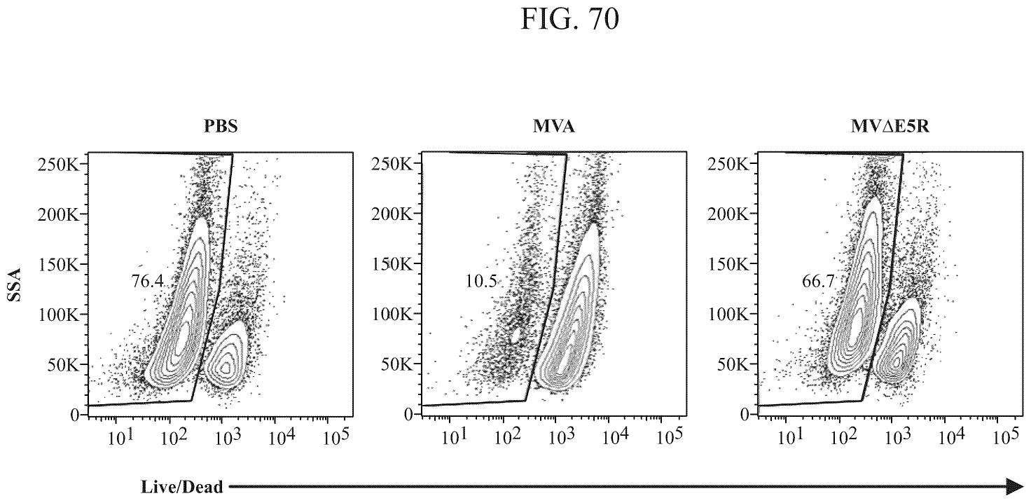

To quantify the fraction of MVAΔE5R-infected BMDCs that were alive after several days of culture with regular medium compared to MVA-infected BMDCs, BMDCs were infected with either MVA or MVAΔE5R at a MOI of 10. Cells were harvested at 16 h post infection and stained with LIVE/DEAD fixable viability dye and subjected for flow cytometry analysis. FACS results show that whereas 76.4% of PBS-mock infected BMDCs were alive, only 10.5% of BMDCs infected with MVA were alive. By contrast, 66.7% of BMDCs infected with MVAΔE5R were alive at 16 h post infection (FIG. 70).

### Example 71: MVAΔE5R Infection Promotes DC Maturation in a cGAS-Dependent Manner

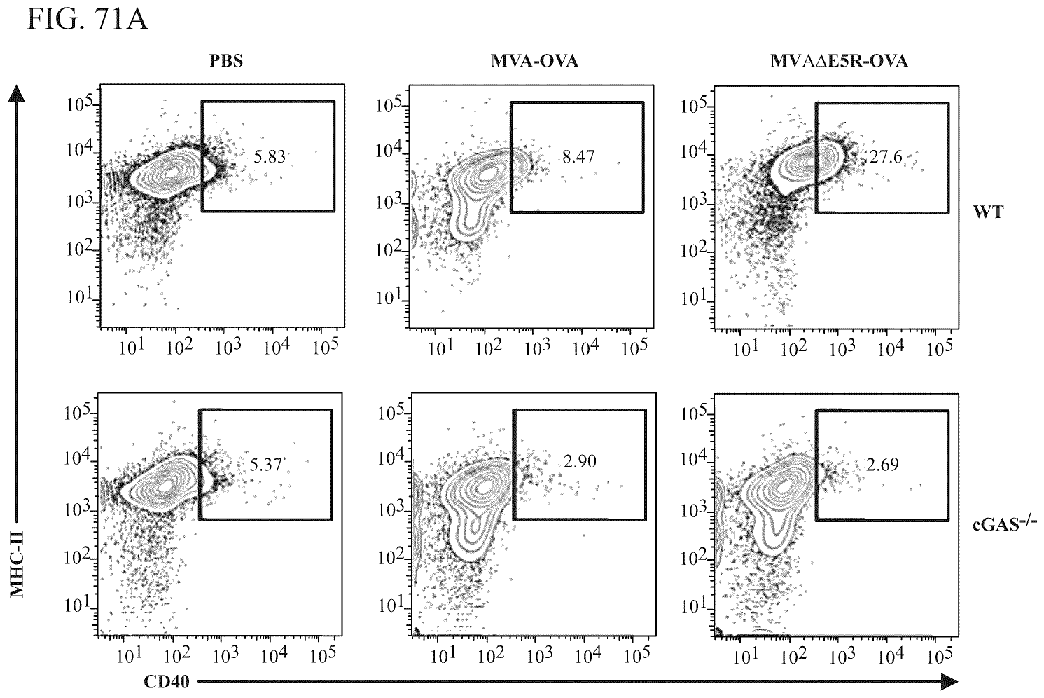

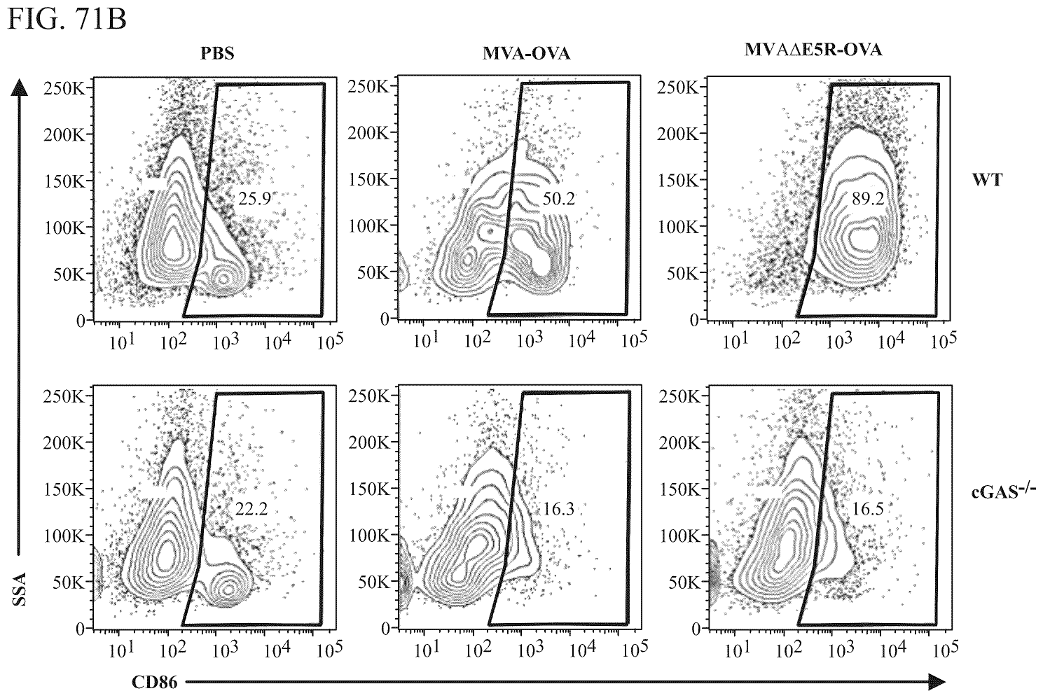

It is known that that BMDCs infected with MVAΔE5R exhibit an activated phenotype, with extension of dendrites. CD40 and CD86 are two known DC activation markers. To determine whether MVAΔE5R infection induces DC activation, and whether DC maturation occurs via a cGAS-dependent mechanism, BMDCs from WT and cGAS^{-/-} mice were either mock infected or infected with MVA-OVA or MVAΔE5R-OVA at MOI of 10. Cells were collected at 16 h post infection and stained for DC maturation markers: CD40 (FIG. 71A) and CD86 (FIG. 71B). BMDCs express high levels of MHCII. Upon MVAΔE5R-OVA infection, CD40 was upregulated on WT BMDCs, but not in cGAS^{-/-} BMDCs (FIG. 71A). MVAΔE5R-OVA infection of BMDCs strongly upregulated CD86 expression on WT BMDCs, but not in cGAS^{-/-} BMDCs. 89.2% of WT BMDCs were CD86⁺ after MVAΔE5R infection, whereas only 16.5% of cGAS^{-/-} BMDCs were CD86⁺ after MVAΔE5R infection. MVA infection in WT BMDCs resulted in 50.2% CD86⁺ BMDCs, but only 16.3% CD86⁺ cGAS^{-/-} BMDCs (FIG. 71B). These results demonstrate that MVAΔE5R infection of BMDCs induces DC maturation, as manifested as upregulation of maturation markers, including CD40 and CD86, in a cGAS-dependent manner.

### Example 72: MVAΔE5R Infection of BMDCs Promotes Antigen Cross-Presentation as Measured by T Cell Activation

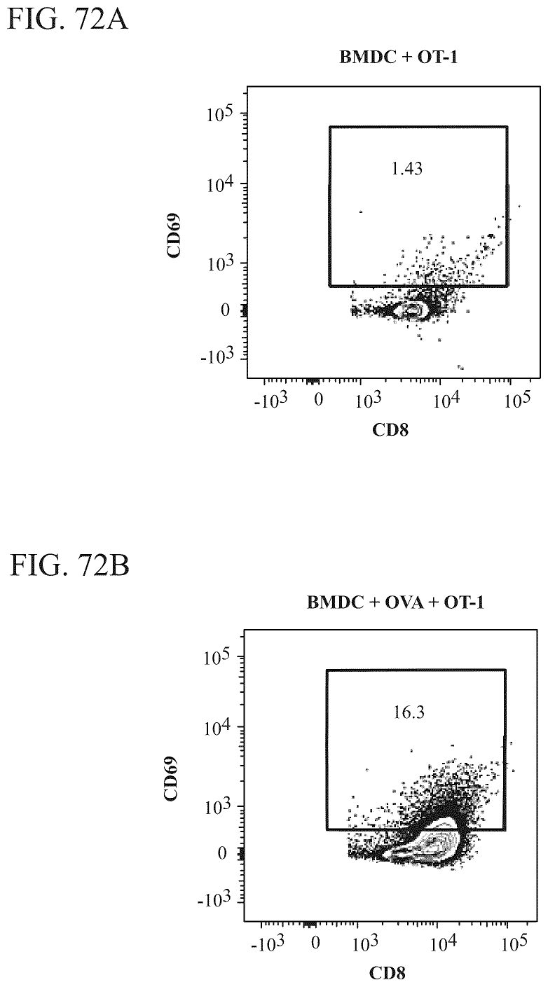

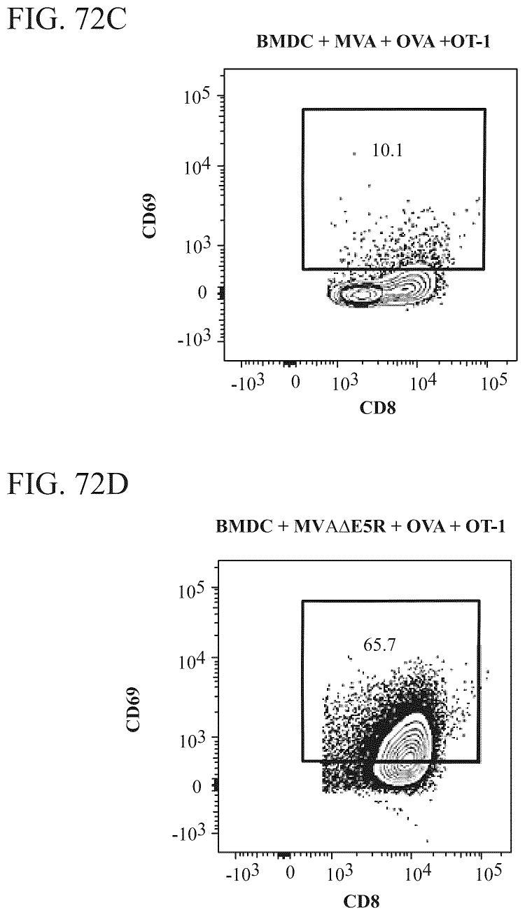

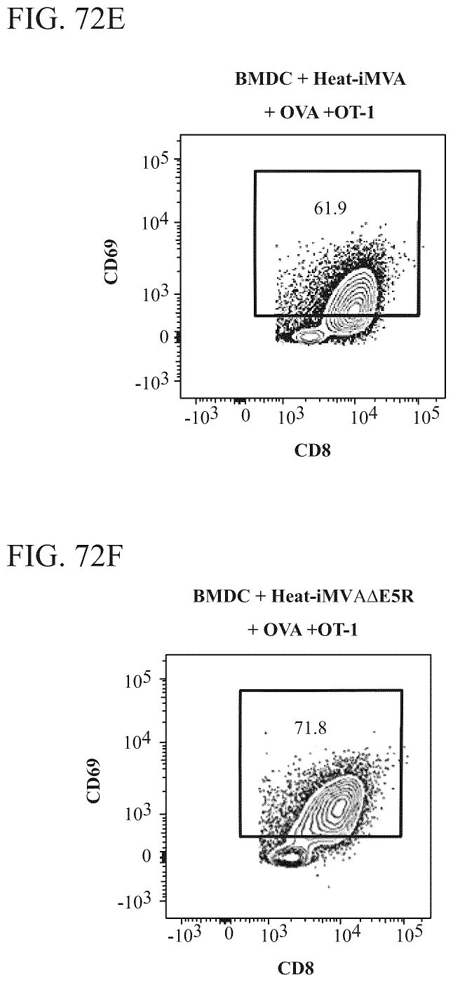

To test the functional significance of BMDC activation induced by MVA, MVAΔE5R, Heat-iMVA, or Heat-iMVAΔE5R, BMDCs were infected with either MVA, MVAΔE5R, Heat-iMVA, or Heat-iMVAΔE5R at MOI of 3 for 3 h and then incubated with chicken ovalbumin (OVA) for 3 h. The OVA protein was washed away and cells were then incubated with OT-I cells (which recognizes OVA₂₅₇₋₂₆₄ SIINFEKL peptide) for 3 days. The BMDC: OT-1 cell ratio was 1:1. OT-1 cells were stained with anti-CD69 and anti-CD8 antibodies and analyzed by flow cytometry. Dot plots demonstrate CD8⁺ cells expressing CD69 (FIGS. 72A-72G). The results show that MVAΔE5R-infected OVA-pulsed BMDCs:OT-1 T cell co-culture lead to 65.7% CD69⁺CD8⁺ T cells, whereas MVA-infected OVA-pulsed BMDCs:OT-1 T cell co-culture resulted in 10.1% CD69⁺CD8⁺ T cells. Heat-iMVA or Heat-iMVAΔE5R infection of BMDCs also resulted in activation of OT-1 T cells in the OVA-pulsed BMDC:OT-1 T cell co-culture. These results demonstrate that MVAΔE5R, Heat-iMVA, or Heat-iMVAΔE5R infection of BMDCs promotes antigen cross-presentation by BMDCs.

### Example 73: MVAΔE5R Infection of BMDCs Promotes Antigen Cross-Presentation as Measured by IFN-γ Production by Activated T Cells

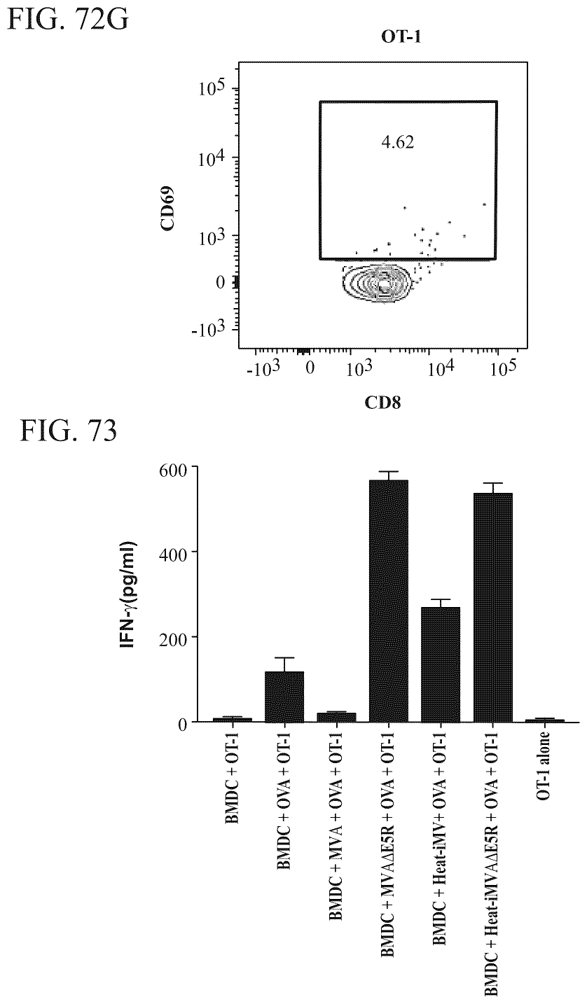

Supernatants were collected at the end of the 3 day BMDC:OT-1 T cell co-culture in the experiment outlined in Example 72. IFN-□ levels in the supernatants were determined by ELISA. The results demonstrate that either MVAΔE5R or Heat-iMVAΔE5R-infected OVA-pulsed BMDC:OT-1 T cell co-cultures generated high levels of IFN-□ protein in the supernatants (FIG. 73). These results indicate MVAΔE5R or Heat-iMVAΔE5R infection of BMDCs promotes antigen cross-presentation by BMDCs.

### Example 74: VACVΔE5R Infection of BMDCs Promotes Antigen Cross-Presentation as Measured by IFN-γ Production by Activated T Cells

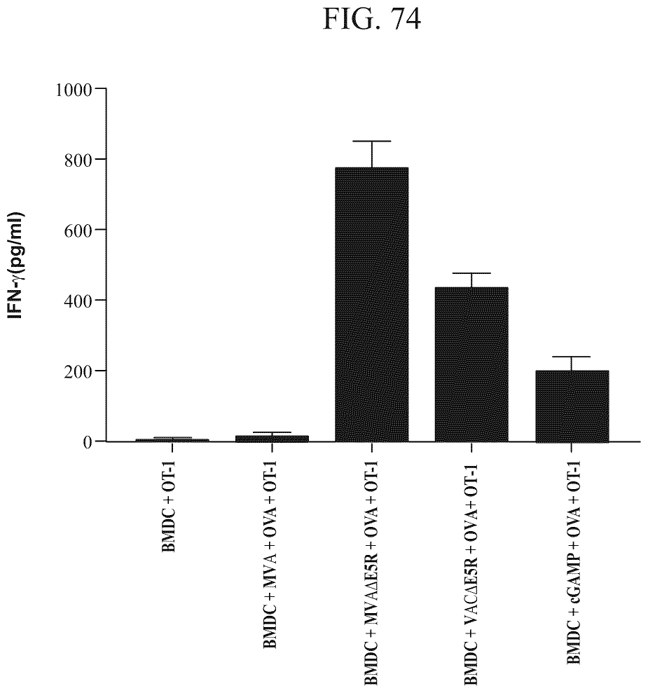

It was previously observed that VACVΔE5R infection of BMDCs induces higher levels of IFNB gene expression and IFN-□ protein production than MVA (FIGS. 56A and 56B). To determine whether VACVΔE5R infection of BMDCs promotes antigen cross-presentation, the inventors performed the following experiment. Briefly, BMDCs were infected with either MVA, MVAΔE5R, or VACVΔE5R at MOI of 3 for 3 h; or BMDCs were incubated with cGAMP (20 μM) or mock control for 3h. BMDCs were subsequently incubated with OVA for 3 h and then the OVA protein was washed away. Cells were then incubated with OT-I cells (which recognizes OVA₂₅₇₋₂₆₄ SIINFEKL peptide) for 3 days. Supernatants were collected and IFN-□□ levels were determined by ELISA (FIG. 74). The results demonstrate that MVAΔE5R-infected OVA-pulsed BMDC:OT-1 T cell co-culture generated highest level of IFN-□ in the supernatants. VACVΔE5R-infected OVA-pulsed BMDC:OT-1 T cell co-culture secreted higher level of IFN-□ compared with cGAMP-treated OVA-pulsed BMDC:OT-1 T cell co-culture. These results indicate that VACVΔE5R infection of BMDCs also promotes antigen cross-presentation.

### Example 75: Deletion of the E5R Gene from MVA Genome Improves Vaccination Efficacy

MVA is an important and safe vaccine vector. Given that MVA has modest induction of IFN-□□ secretion from BMDCs and modest activation effects on DC maturation, identification of immune suppressive mechanism can lead to improvement of MVA-based vaccine vector design. To test whether deletion of the E5R gene from MVA improves vaccination efficacy, the inventors performed the following experiment. Briefly, on day 0, C57BL/6J mice were vaccinated with MVA-OVA or MVAΔE5R-OVA at 2×10⁷ pfu either through skin scarification or intradermal injection (FIG. 75A). Spleens were harvested from euthanized mice one week later and co-cultured with OVA₂₅₇₋₂₆₄ (SIINFEKL) peptide (10 g/ml) pulsed BMDCs for 12 h. The intracellular IFN-γ levels in CD8⁺ T cells was then measured by flow cytometry. (* p<0.05; ** p<0.01). FIG. 75B shows activated CD8⁺ T cells after vaccination through skin scarification with MVA-OVA or MVAΔE5R-OVA. FIG. 75C shows activated CD8⁺ T cells after vaccination through intradermal injection of MVA-OVA or MVAΔE5R-OVA. These results demonstrate that vaccination with MVAΔE5R-OVA either through skin scarification or intradermal injection leads to higher activated CD8⁺ T cells compared with vaccination with MVA-OVA. Therefore, removing the E5R gene from MVA genome improves vaccination efficacy.

### Example 76: MVAΔE5R Infection Induces IFNB Gene Expression and IFN-β Secretion from Murine Primary Fibroblasts in a cGAS-Dependent Manner

In addition to dendritic cells and macrophages, the inventors investigated whether MVAΔE5R infection of skin dermal fibroblasts also induce type I IFN production. Skin dermal fibroblasts were generated from female WT and cGAS' C57BL/6J mice. Cells were infected with either MVA, MVAΔE5R, Heat-iMVA, or Heat-iMVAΔE5R. Cells and supernatants were collected at 16 h post infection. RT-PCR results showed that MVAΔE5R infection triggered IFNB gene expression in WT dermal fibroblasts but not in cGAS^{-/-} cells (FIG. 76A). ELISA results demonstrated MVAΔE5R induced IFN-β protein secretion from MVAΔE5R-infected WT dermal fibroblasts but not in infected cGAS^{-/-} cells (FIG. 76B). By contrast, MVA infection of WT induced lower level of IFN-β protein secretion from WT dermal fibroblasts (FIG. 76B). The IFN-β protein concentration in the supernatants of MVA-infected WT dermal fibroblasts was 350 μg/ml, compared with 10970 μg/ml in the supernatants of MVAΔE5R-infected WT dermal fibroblasts. These results demonstrate that E5 is a potent inhibitor of IFN production in MVA-infected dermal fibroblasts, and MVAΔE5R generates strong immune activating effect on skin dermal fibroblasts. This immune activating effect is likely to contribute to its enhanced vaccine efficacy through skin scarification or intradermal infection. In addition, MVAΔE5R is likely to activate tumor stromal cells or cancer-associated fibroblasts through similar mechanisms, which would contribute to its effect on altering tumor-immune suppressive microenvironment.

### Example 77: MVAΔE5R Gains its Capacity to Replicate its DNA in cGAS- or IFNAR1-Deficient Skin Primary Dermal Fibroblasts

To test whether cGAS or IFNAR1 contribute to host restriction of MVA or MVAΔE5R virus in skin dermal fibroblasts, skin primary dermal fibroblasts from WT, cGAS^{-/-} or IFNAR1^{-/-} mice were infected with either MVA or MVAΔE5R at a MOI of 3. Cells were collected 1, 4, 10 and 24 h post infection. Viral DNA copy numbers were determined by quantitative PCR. Although MVA has limited capacity of replicating viral genome in WT dermal fibroblasts, its replication capacity increased dramatically in cGAS^{-/-} cells and modestly in IFNAR1^{-/-} cells (FIGS. 77A and 77B). For example, MVA DNA copy number increased from 2-fold at 4 h, to 28-fold at 10 h, and to 41-fold at 24 h post infection compared with that at 1 h post infection in WT dermal fibroblasts. In cGAS^{-/-} cells, MVA DNA copy number increased from 8-fold at 4 h, to 120-fold at 10 h, and to 390-fold at 24 h post infection compared with that at 1 h post infection. In IFNAR1^{-/-} cells, MVA DNA copy number increased from 5-fold at 4 h, to 50-fold at 10 h, and to 107-fold at 24 h post infection compared with that at 1 h post infection (FIGS. 77A and 77B).

Similarly, in WT dermal fibroblasts, MVAΔE5R DNA copy number increased from 1-fold at 4 h, to 18-fold at 10 h, and 21-fold at 24 h post infection compared with that at 1 h post infection. In cGAS^{-/-} cells, MVAΔE5R DNA copy number increased from 1.8-fold at 4 h, to 87-fold at 10 h, and to 832-fold at 24 h post infection compared with that at 1 h post infection. In IFNAR1^{-/-} cells, MVAΔE5R DNA copy number increased from 1.9-fold at 4 h, to 57-fold at 10 h, and to 181-fold at 24 h post infection compared with that at 1 h post infection (FIGS. 77C and 77D). These results demonstrate that the cGAS-mediated sensing mechanism in dermal fibroblasts was critical in controlling MVA and MVAΔE5R viral DNA replication, and the IFNAR-mediated type I IFN positive feedback loop plays a partial role.

### Example 78: MVAΔE5R Gains its Capacity to Generate Infectious Progeny Viruses in cGAS-Deficient Skin Primary Dermal Fibroblasts

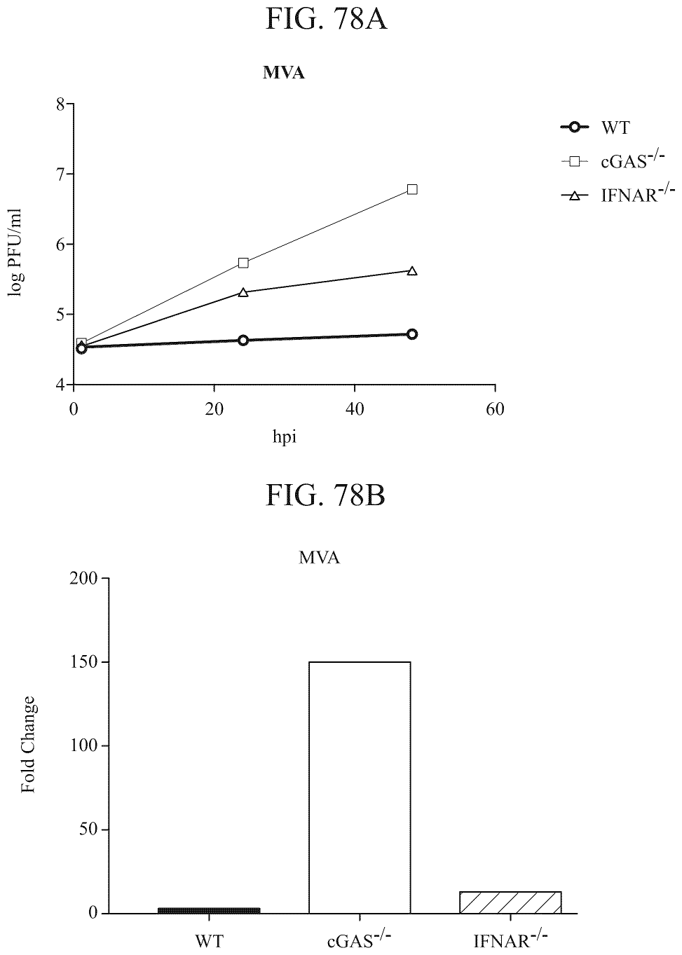

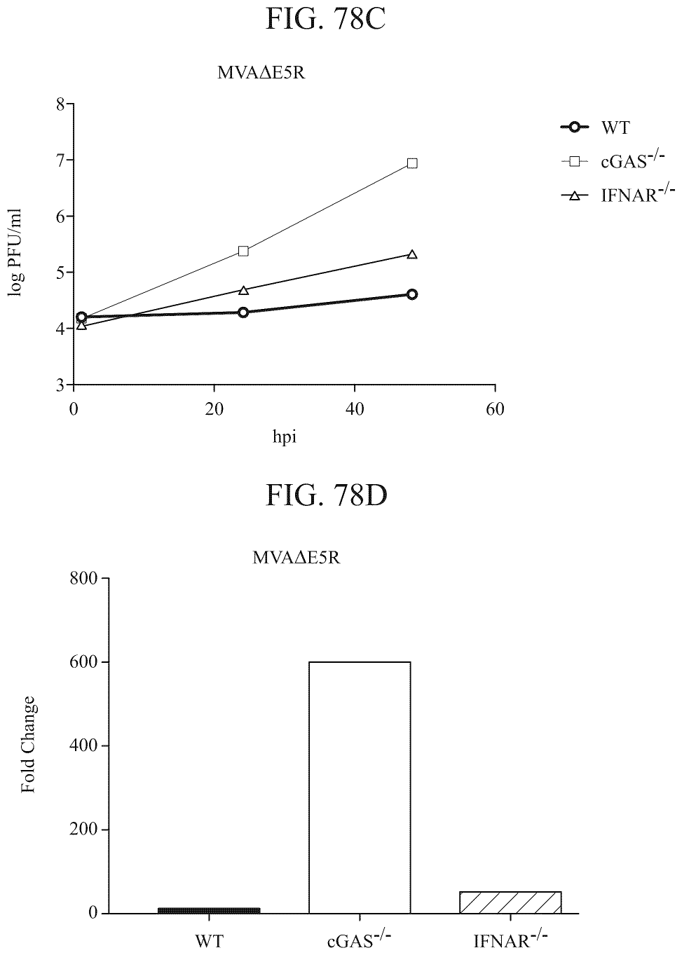

MVA is non-replicative in dermal fibroblasts. To determine whether the cGAS-mediated cytosolic DNA-sensing pathway and the IFANR pathway play a role in restricting the production of infectious virions, skin primary dermal fibroblasts from WT, cGAS^{-/-} or IFNAR1^{-/-} mice were infected with either MVA or MVAΔE5R at a MOI of 0.05. Cells were collected 1, 24, and 48 h post infection. Viral titers were determined by titrating on BHK21 cells. In WT dermal fibroblasts, both MVA and MVAΔE5R are non-replicative. However, in cGAS^{-/-} cells, MVA titers at 48 h post infection increased by 148-fold compared with its titers at 1 h post infection (FIGS. 78A and 78B). More strikingly, MVAΔE5R titers at 48 h post infection increased by 583-fold compared with its titers at 1 h post infection (FIGS. 78C and 78D). In IFNAR1^{-/-} cells, MVA and MVAΔE5R increased their titers at 48 h post infection by 12- and 19-fold respectively compared with their titers at 1 h post infection (FIGS. 78A-77D). These results demonstrate that cGAS plays a critical role in restricting both MVA and MVAΔE5R replication in skin dermal fibroblasts.

### Example 79: MVAΔE5R Infection of Murine Melanoma Cells Induce IFNB Gene Expression and IFN-β Protein Secretion in a STING-Dependent Manner

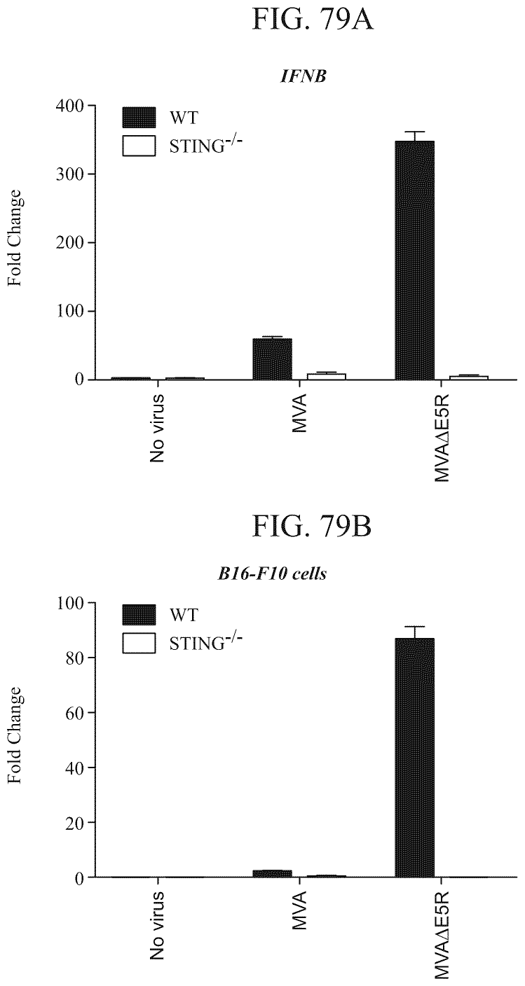

To determine whether MVAΔE5R infection of tumor cells induces IFNB gene expression and IFN-β protein secretion, WT and STING^{-/-} B16-F10 cells (generated by CRISPR-cas9 gene targeting of STING) were infected with either MVA or MVAΔE5R at a MOI of 10. Cells were collected at 18 h post infection. RNAs were extracted and quantitative real-time PCR analysis was performed. The RT-PCR results demonstrate that MVAΔE5R induced IFNB gene expression in WT B16-F10 cells, but not in STING^{-/-} cells. MVA infection demonstrated a weaker induction of IFNB gene expression compared with MVAΔE5R in WT B16-F10 cells (FIG. 79A). ELISA results of IFN-β protein levels in the supernatants of WT and STING^{-/-} B16-F10 cells infected with either MVA or MVAΔE5R collected at 18 h post infection demonstrated MVAΔE5R induces modest level of IFN-β protein secretion from WT B16-F10 cells, but not in STING^{-/-} cells (FIG. 79B). MVA infection did not result in IFN-β protein secretion (FIG. 79B).

### Example 80: MVAΔE5R Infection of Murine Melanoma Cells Induces ATP Release, which is a Hallmark of Immunogenic Cell Death

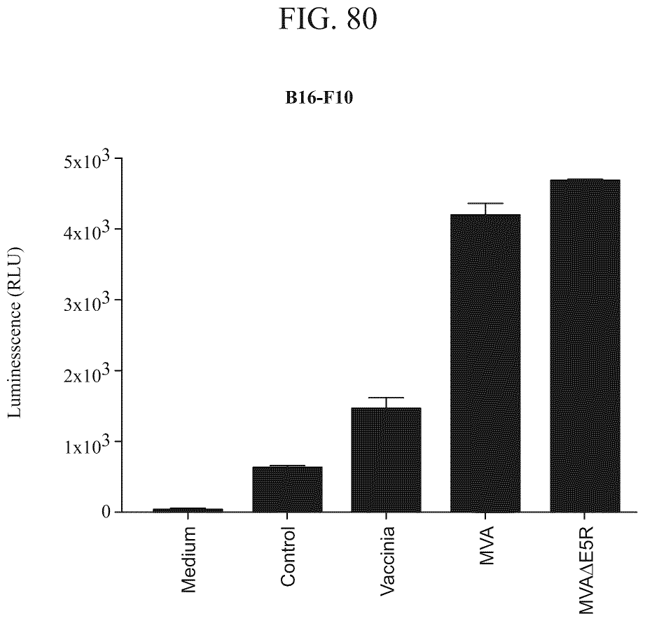

FIG. 80 demonstrates that MVAΔE5R infection of murine melanoma cells induces ATP release, which is a hallmark of immunogenic cell death. Briefly, B16-F10 cells were infected with WT vaccinia, MVA, or MVAΔE5R at a MOI of 10. Cells were washed and fresh medium was added one hour after virus infection. Supernatants were collected at 48 h post infection. ATP levels were determined by using ATPlite 1step Luminescence ATP Detection Assay System (PerkinElmer, Waltham, MA). The results showed that MVA and MVAΔE5R infection of murine melanoma cells induced higher ATP release than vaccinia virus, which demonstrate that MVA and recombinant MVA induced immunogenic cell death in tumor cells. Future studies will examine whether MVA or MVAΔE5R infection of tumor cells also induce HMGB1 release, another hallmark of immunogenic cell death.

### Example 81: Generation of Recombinant MVAΔE5R Expressing hFlt3L and hOX40L

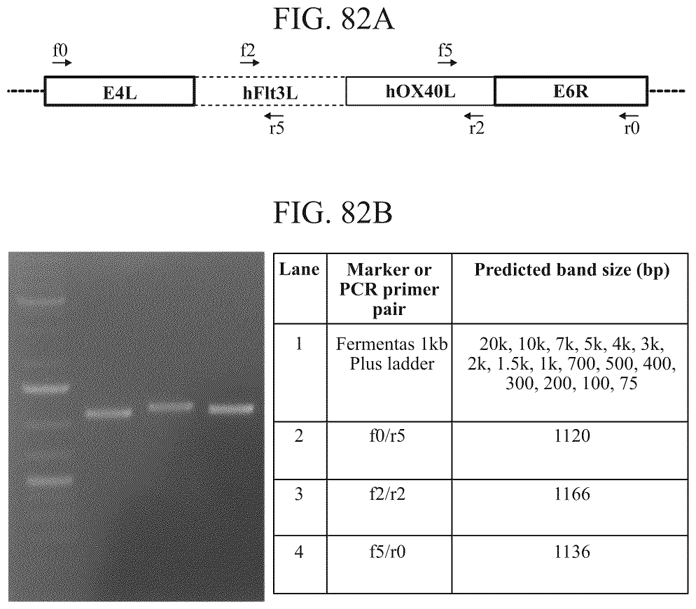

This example describes the generation of recombinant MVAΔE5R virus expressing hFlt3L and hOX40L. FIG. 81 shows a scheme of generating recombinant MVAΔE5R expressing hFlt3L and hOX40L through homologous recombination at the E4L and E6R loci of MVA genome. pMA vector was used to insert a single expression cassette designed to express both hFlt3L and hOX40L using the vaccinia viral synthetic early and late promoter (PsE/L). The coding sequence of the hFlt3L and hOX40L was separated by a cassette including a furin cleavage site followed by a Pep2A sequence. Homologous recombination that occurred at the E4L and E6R loci resulted in the insertion of expression cassette for hFlt3L and hOX40L. FIGS. 82A and 82B demonstrate that the recombinant MVAΔE5R-hFlt3L-hOX40L virus has the expected insertion as determined by PCR analysis. The DNA insert was also subjected to Sanger Sequencing and the sequences were as expected.

### Example 82: Expression of hFlt3L and hOX40L by Cells Infected with MVAΔE5R-hFlt3L-hOX40L

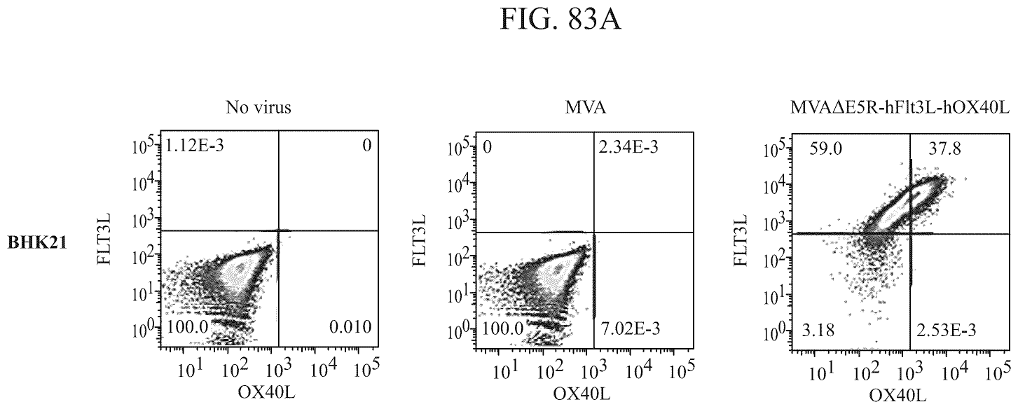

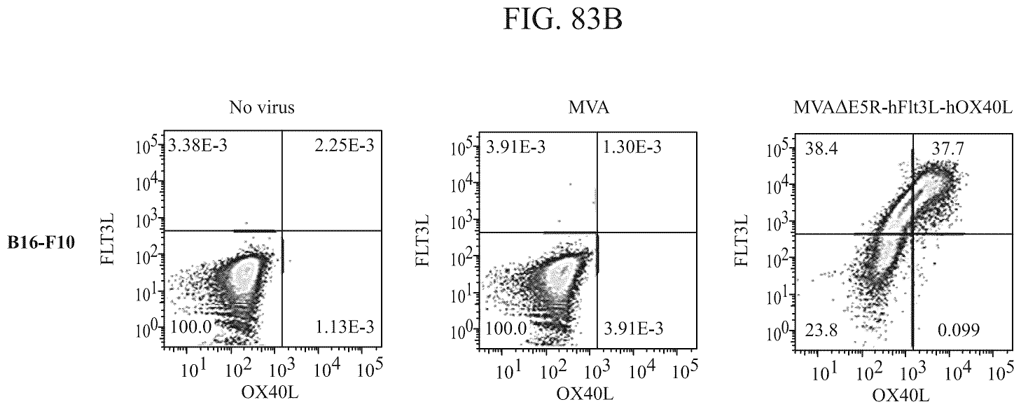

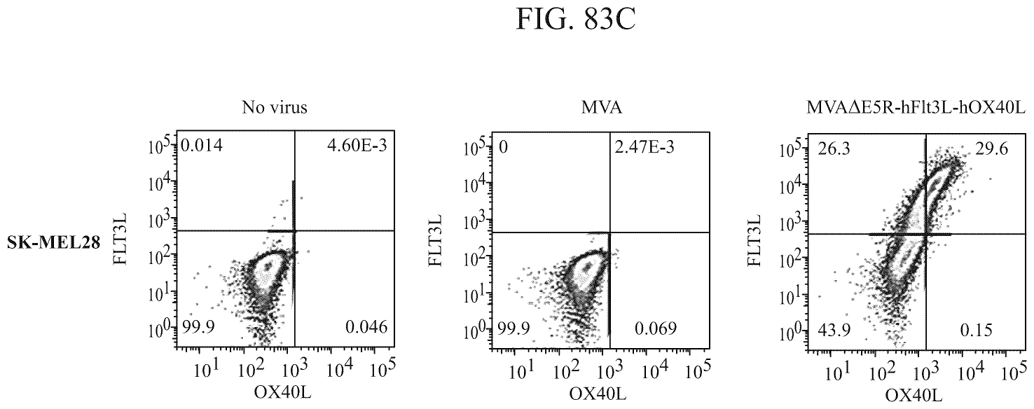

To test whether the recombinant MVAΔE5R-hFlt3L-hOX40L virus expresses both hFlt3L and hOX40L on the surface of infected cells, BHK-21, murine B16-F10 melanoma cells and human SK-MEL28 melanoma cells were plated and infected with either MVA or MVAΔE5R-hFlt3L-hOX40L at MOI 10. A no virus, mock infection control was included. 24 hours post infection, cells were harvested for surface staining with hFlt3L and hOX40L antibodies. Surface expression of hFlt3L and hOX40L was analyzed by FACS analysis. FIG. 83A are dot plots of surface expression of hFlt3L and hOX40L in BHK-21 cells after infection. FIG. 83B are dot plots of surface expression of hFlt3L and hOX40L in B16-F10 cells after infection. FIG. 83C are dot plots of surface expression of hFlt3L and hOX40L in SK-MEL28 cells after infection. The results demonstrate that MVAΔE5R-hFlt3L-hOX40L expressed hOX40L and hFlt3L efficiently in BHK-21, B16-F10 and SK-MEL28 cells. Given that MVAΔE5R-hFlt3L-hOX40L does not replicate in B16-F10 or SK-MEL28, the expression of hFlt3L and hOX40L on the infected tumor cells was robust.

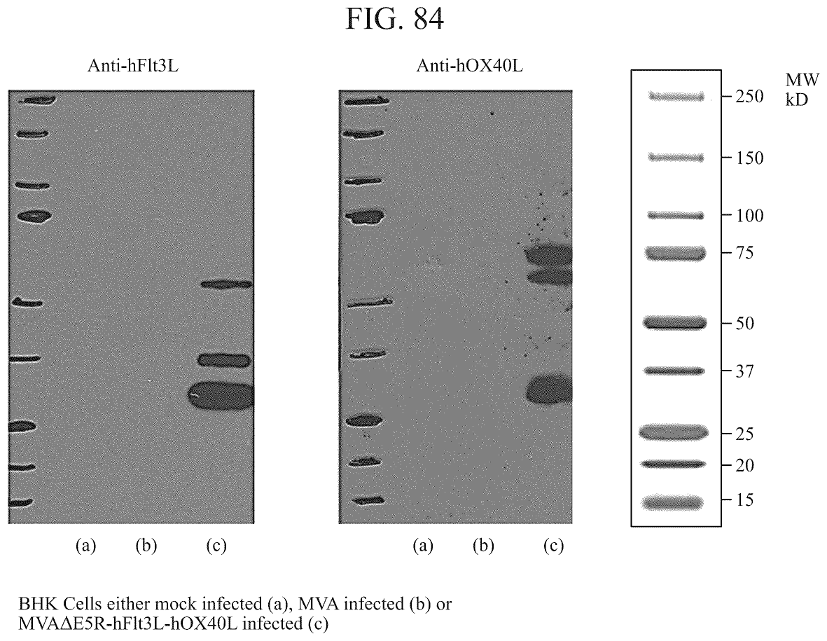

Western blot analysis was performed to test whether MVAΔE5R-hFlt3L-hOX40L virus expresses hFlt3L and hOX40L on BHK21 cells (FIG. 84). Briefly, BHK21 cells were either mock infected or infected with MVA or MVAΔE5R-hFlt3L-hOX40L. Cell lysates were collected at 24 h post infection. Western blot results show that hFlt3L and hOX40L were expressed by MVAΔE5R-hFlt3L-hOX40L-infected BHK21 cells, but not by MVA (FIG. 84).

### Example 83: Generation of Recombinant VACV-TK⁻-Anti-CTLA-4-ΔE5R-hFlt3L-mOX40L

This example describes the generation of a recombinant vaccinia or MVA virus expressing an antibody that selectively targets cytotoxic T lymphocyte antigen 4 at the TK locus and expressing human Flt3L and murine OX40L genes in the E5R locus (VAC-TK⁻-anti-muCTLA-4-E5R⁻-hFlt3L-mOX40L). FIG. 85A shows the schematic diagram of a single expression cassette designed to express the heavy chain and light of the antibody using the vaccinia viral synthetic early and late promoter (PsE/L). The coding sequence of the heavy chain (muIgG2a) and the light chain of 9D9 were separated by a cassette including a furin cleavage site followed by a 2A peptide (Pep2A) sequence to enable ribosome skipping. A pCB plasmid was constructed which contained the anti-mu-CTLA-4 gene under the control of the vaccinia PsE/L as well as the E. coli xanthine-guanine phosphoribosyl transferase gene (gpt) under the control of vaccinia P7.5 promoter flanked by the thymidine kinase (TK) gene on either side. Recombinant virus expressing anti-mu-CTLA-4 from TK locus was generated through homologous recombination at the TK locus between pCB plasmid DNA and viral genomic DNA. FIG. 85B shpws the schematic diagram which used the vaccinia viral synthetic early and late promoter (PsE/L) to express both human Flt3L and murine OX40L as a fusion protein in a single expression cassette. The coding sequence of human Flt3L and murine OX40L was separated by a cassette including a furin cleavage site followed by a 2A peptide (Pep2A) sequence, which enabled ribosome skipping. A pUC57 plasmid was constructed which contained human Flt3L and murine OX40L fusion gene flanked by the E4L and E6R genes on either side. Recombinant virus expressing human Flt3L and murine OX40L fusion protein from E5R locus was generated through homologous recombination at E5R locus between pUC57 plasmid DNA and viral genomic DNA. To generate the VAC-TK⁻-anti-muCTLA-4-E5R⁻-hFlt3L-mOX40L, first BSC-40 cells were infected with vaccinia virus at a multiplicity of infection (MOI) of 0.05 for 1 h, and were then transfected with the pCB plasmid DNAs described above. The infected cells were collected at 48 h. Recombinant viruses were selected through further culturing in gpt selection medium including MPA, xanthine and hypoxanthine, and plaque purified. PCR analysis was performed to identify recombinant viruses with deletion of part of the TK gene and with anti-muCTLA-4 insertion. Homologous recombination that occurred at the TK locus resulted in the insertion of anti-mu-CTLA-4 and gpt expression cassettes into the viral genomic DNA to generate VAC-TK⁻-anti-muCTLA-4. On the second step, BSC-40 cells were infected with VAC-TK⁻-anti-muCTLA-4 at a MOI of 0.1 for 1 h, and then were transfected with the pUC57 plasmid DNAs described above. The infected cells were collected at 48 h. Recombinant viruses were selected through GFP marker and plaque purification. PCR analysis was performed to identify recombinant viruses with deletion of the E5R gene and with insertion of human Flt3L and murine OX40L fusion gene to generate VAC-TK⁻-anti-muCTLA-4-E5R⁻-hFlt3L-mOX40L recombinant virus.

### Example 84: PCR Verification of Recombinant VACV-TK⁻-Anti-muCTLA-4-ΔE5R-hFlt3L-mOX40L and the Expression of Anti-muCTLA-4 Antibody by Cells Infected with the Recombinant Virus

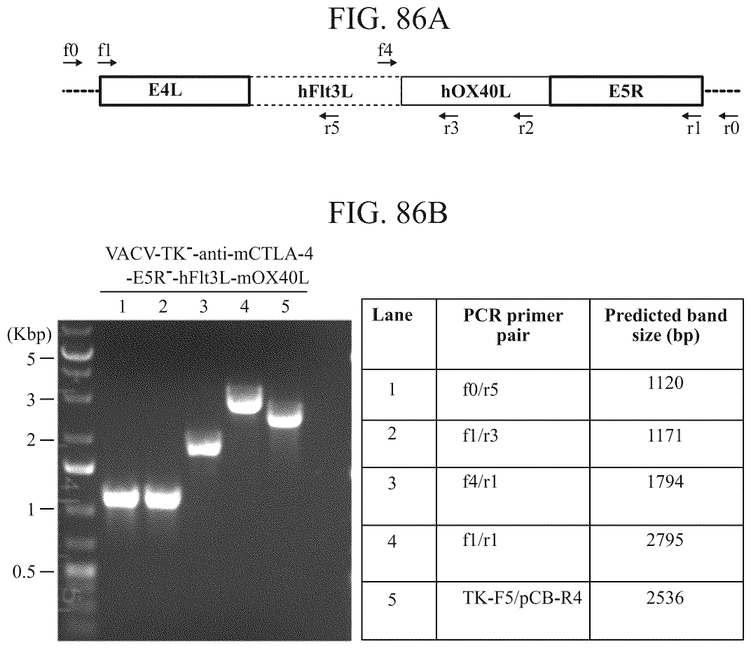

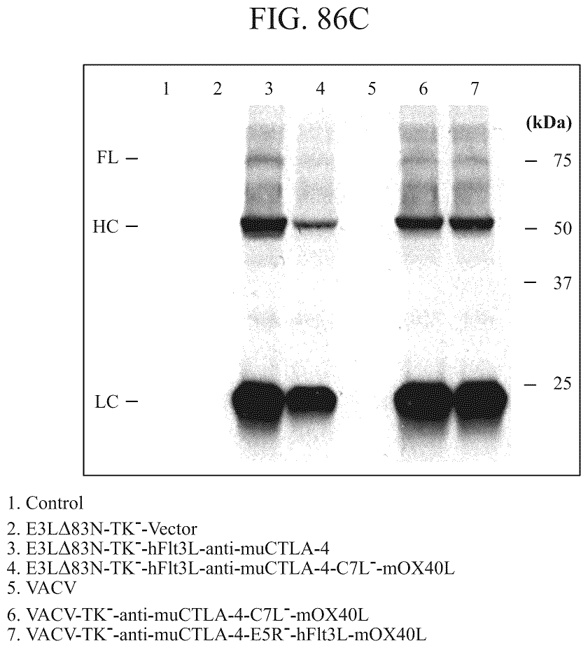

PCR analyses was used to verify the recombinant VACV-TK⁻-anti-muCTLA-4-ΔE5R-hFlt3L-mOX40L (FIGS. 86A and 86B). To determine whether recombinant virus infection results in the production of anti-CTLA-4 antibodies, human SK-MEL-28 melanoma cells were mock infected or infected with E3LΔ83N-TK⁻-vector, E3LΔ83N-TK⁻-hFlt3L-anti-muCTLA-4, E3LΔ83N-TK⁻-hFlt3L-anti-muCTLA-4-C7L⁻-mOX40L, VACV, VAC-TK⁻-anti-muCTLA-4-C7L⁻-mOX40L, or VAC-TK⁻-anti-muCTLA-4-E5R⁻-hFlt3L-mOX40L at a MOI of 10. Cell lysates were collected at 24 h post-infection, and polypeptides were separated using 10% SDS-PAGE. HRP-linked anti-mouse IgG (heavy and light chain) antibody was used to detect full-length (FL), heavy chain (HC), and light chain (LC) of anti-muCTLA-4 antibodies. Western blot analysis shows the expression of the full-length (FL), heavy chain (HC), and light chain (LC) of anti-CTLA-4 antibodies in SK-MEL-28 melanoma cell lines after virus infection (FIG. 86C). Accordingly, these results demonstrate that the recombinant viruses of the present technology have the capacity to express anti-CTLA-4 antibodies in infected cells and are useful in methods for delivering the antibodies to cells.

### Example 85: Vaccinia E5 is Highly Conserved Among the Poxvirus Family

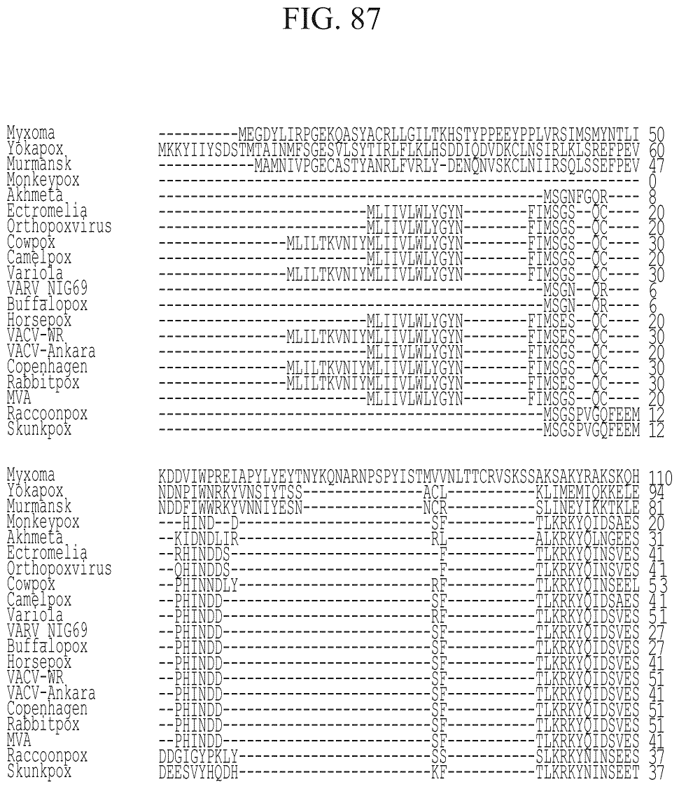

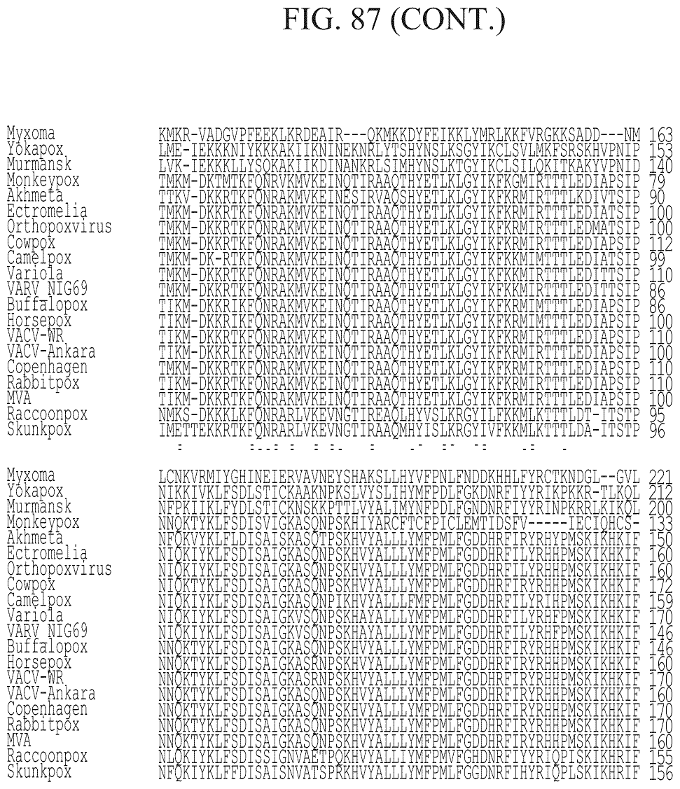

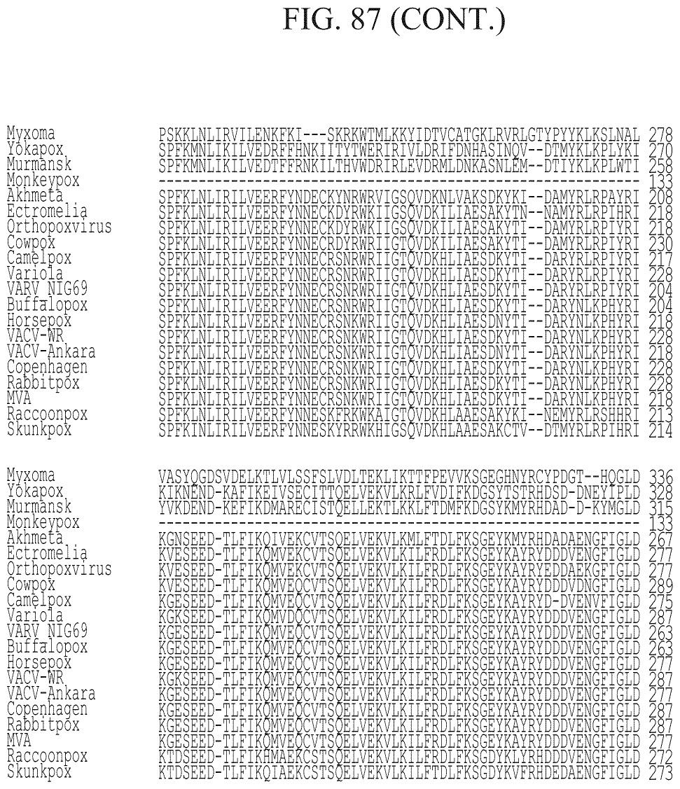

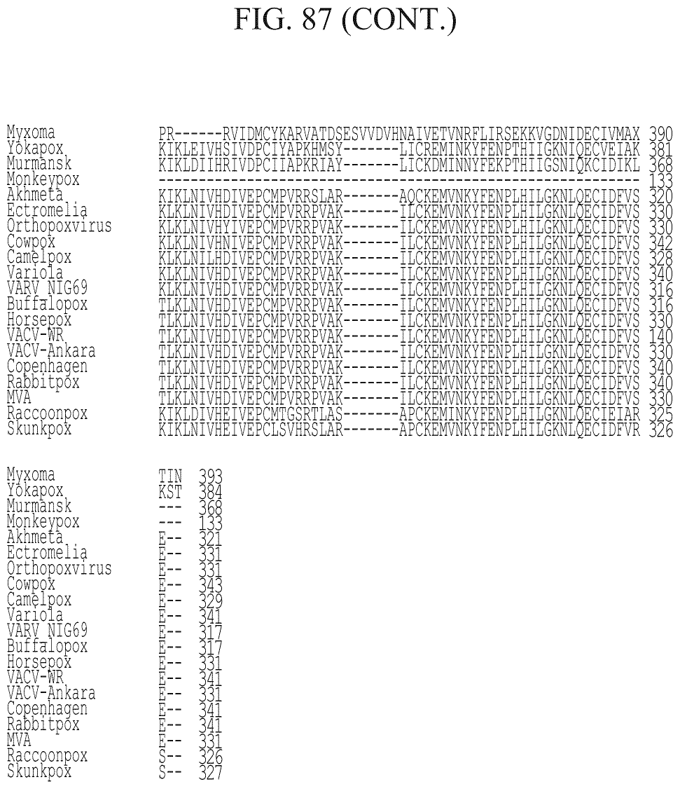

Vaccinia E5 is highly conserved among the poxvirus family. FIG. 87 shows the protein sequence alignments of E5 orthologs from multiple members of the poxvirus family. E5 orthologs exhibit differences in N-terminals but high conservation in middle to C-terminal. FIG. 88A shows the protein sequence alignments of E5 from Vaccinia WR and Modified vaccinia virus Ankara (MVA). MVA lacks the first 10 amino acids and point mutations at N-terminal compared with VACV. FIG. 88B shows the protein sequence alignments of E5 from vaccinia virus and M31, which is the E5 orthologue in Myxoma virus. M31 and E5 from vaccinia virus share about 20% homology.

### Example 86: Myxoma Virus M31, an Ortholog of Vaccinia E5, Inhibits cGAS and STING Induced IFN-β Pathway

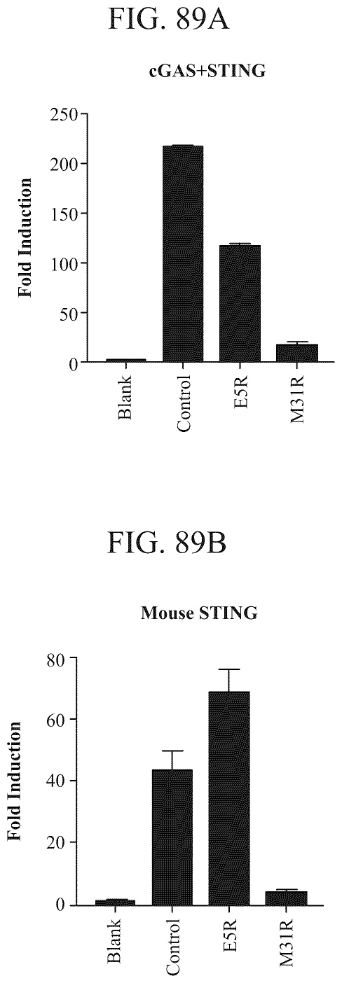

To investigate the role of Myxoma virus M31, an ortholog of vaccinia E5, in cGAS and STING induced IFN-β pathway, HEK293T cells were transfected with plasmids expressing murine cGAS, human STING together with either E5R, M31R or pcDNA vector control expressing plasmids. After 24h, cells were harvest for a luciferase assay. FIG. 89A demonstrated that both E5 and M31 were able to inhibit IFN-β production. Myxoma M31 demonstrated a stronger inhibition effect than E5. FIG. 89B shows that HEK293T were transfected with plasmids expressing murine STING together with either E5R, M31R or pcDNA vector control expressing plasmids. 24h post transfection, the luciferase signal was determined. E5 did not block IFN-β production while M31 still inhibited STING-induced IFN-β production, which demonstrated that M31 may act downstream of the cGAS and STING induced IFN-β pathway.

### Example 87: Vaccinia E5 Promotes cGAS Ubiquitination

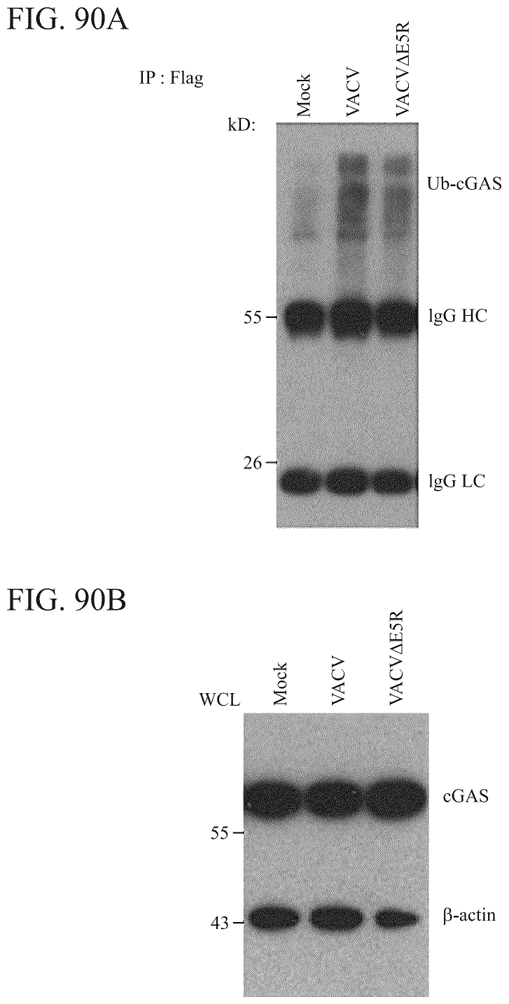

To assess whether E5 blocks cGAS induced IFN-β pathway by promoting cGAS ubiquitination, HEK293T cells were transfected with Flag-cGAS and HA-ubiquitin. After 24 hours, cells were infected with either WT VACV or VACVΔE5R. Cell lysis were collected 6 hpi. cGAS were immunoprecipitated with anti-Flag antibody and ubiquitination was detected by anti-HA antibody (FIG. 90A). FIG. 90B shows a Western Blot analysis of cGAS and 0-actin on whole cell lysates (WCL). VACV induced cGAS ubiquitination after infection while VACVΔE5R induced lower level of cGAS ubiquitination, which demonstrated that vaccinia E5 promotes cGAS ubiquitination.

### Example 88: Intratumoral Delivery of MVAΔE5R Delays Tumor Growth and Prolongs Survival in Murine B16-F10 Melanoma Unilateral Tumor Implantation Model

To test whether IT delivery of MVAΔE5R generates antitumor effects, a unilateral murine B16-F10 tumor implantation model was used. Briefly, 5×10⁵ B16-F10 melanoma cells were implanted intradermally to right flanks of C57B/6 mice. Eight days after tumor implantation, the tumors were injected with PBS or 4×10⁷ pfu of MVA, MVAΔE5R or Heat-iMVA twice a week. Tumor sizes were measured twice a week and mice survival was monitored (FIG. 91A). The mice survival rate is shown in FIG. 91B. In mice treated with PBS, B16-F10 tumors grew rapidly, which resulted in early death with a median survival of 11 days. Intratumoral injection of MVAΔE5R had superior anti-tumor effect than MVA, which resulted in delayed tumor growth and improved survival. IT MVAΔE5R received equivalent anti-tumor effect to Heat-iMVA with 60% of mice survived in both MVAΔE5R and Heat-iMVA treated group.

### Example 89: MVAΔC7L-hFlt3L-TK(−)mOX40LΔE5R Infection of BMDCs Results in Higher Levels of IFNB Gene Expression and IFN-□ Protein Secretion Compared with MVAΔE5R

FIGS. 92A-C show that MVAΔC7L-hFlt3L-TK(−)mOX40LΔE5R infection of BMDCs results in higher levels of IFNB gene expression and IFN-β protein secretion compared with MVAΔE5R. FIG. 92A-92C shows a schematic diagram of generating MVAΔC7L-hFlt3L-TK(−)-mOX40LΔE5R virus. The first step involved the generation of MVAΔC7L-hFlt3L through homologous recombination at the C8L and C6R loci, replacing C7L gene with hFlt3L under the control of PsE/L promoter. The second step involved the generation of MVAΔC7L-hFlt3L-TK(−)-mOX40L through homologous recombination at the TK loci, replacing TK gene with mOX40L under the control of PsE/L promoter. The resulting virus was described in FIGS. 5A and 5B. The third step was to generate MVAΔC7L-hFlt3L-TK(−)-mOX40LΔE5R through homologous recombination at the E4L and E6R loci, which replaced the E5R gene with mCherry under the control of P7.5 promoter. FIG. 92D shows RT-PCR results of IFNB gene expression in BMDCs infected with either MVAΔE5R, MVAΔC7L-hFlt3L-TK(−)-mOX40L, or MVAΔC7L-hFlt3L-TK(−)mOX40LΔE5R. WT and IFNAR^{-/-} BMDCs were mock-infected or infected with MVAΔE5R, MVAΔC7L-hFlt3L-TK(−)-mOX40L, or MVAΔC7L-hFlt3L-TK(−)mOX40LΔE5R at a MOI of 10. Cells were collected at 16 h post infection and RT-PCR was performed. FIG. 92E shows ELISA results of IFN-β protein levels in the supernatants of BMDCs infected with either MVAΔE5R, MVAΔC7L-hFlt3L-TK(−)-mOX40L, or MVAΔC7L-hFlt3L-TK(−)mOX40LΔE5R. WT and IFNAR^{-/-} BMDCs were mock-infected or infected with MVAΔE5R, MVAΔC7L-hFlt3L-TK(−)-mOX40L, or MVAΔC7L-hFlt3L-TK(−)mOX40LΔE5R at a MOI of 10. Supernatants were collected at 16 h post infection and ELISA was performed to measure IFN-β protein levels. MVAΔC7L-hFlt3L-TK(−)-mOX40LΔE5R induced highest IFN-β production in both RNA level and protein secretion in WT BMDCs compared with MVAΔE5R and MVAΔC7L-hFlt3L-TK(−)-mOX40L. In IFNAR^{-/-} BMDCs, IFN-β production induced by MVAΔC7L-hFlt3L-TK(−)-mOX40LΔE5R was much lower than that in WT BMDCs, which demonstrated that MVAΔC7L-hFlt3L-TK(−)-mOX40LΔE5R induced IFN-β production is partially dependent on the IFNAR pathway.

### Example 90: Generating MVAΔC7LΔE5R-hFlt3L-mOX40L and MVAΔC7L-OVA-ΔE5R-hFlt3L-mOX40L

FIGS. 93A and 93B shows the scheme of generating MVAΔC7LΔE5R-hFlt3L-mOX40L and MVAΔC7L-OVA-ΔE5R-hFlt3L-mOX40L. FIG. 93A shows the scheme of generating MVAΔC7LΔE5R-hFlt3L-mOX40L. pMA plasmid was constructed to use the vaccinia viral synthetic early and late promoter (PsE/L) to express both human Flt3L and murine OX40L as a fusion protein in a single expression cassette. The coding sequence of human Flt3L and murine OX40L was separated by a cassette including a furin cleavage site followed by a 2A peptide (Pep2A) sequence. Recombinant virus expressing human Flt3L and murine OX40L fusion protein from E5R locus was generated through homologous recombination at E4L and E6R loci between pMA plasmid and MVAΔC7L viral genome. FIG. 93B shows the scheme of generating MVAΔC7L-OVA-ΔE5R-hFlt3L-mOX40L. Recombinant virus expressing human Flt3L and murine OX40L fusion protein from E5R locus was generated through homologous recombination at E4L and E6R loci between pMA plasmid and MVAΔC7L-OVA viral genome.

### Example 91: Myxoma M64 has an Inhibitory Role of IFN-β-Induced IFN-Sensitive Response Element Activation

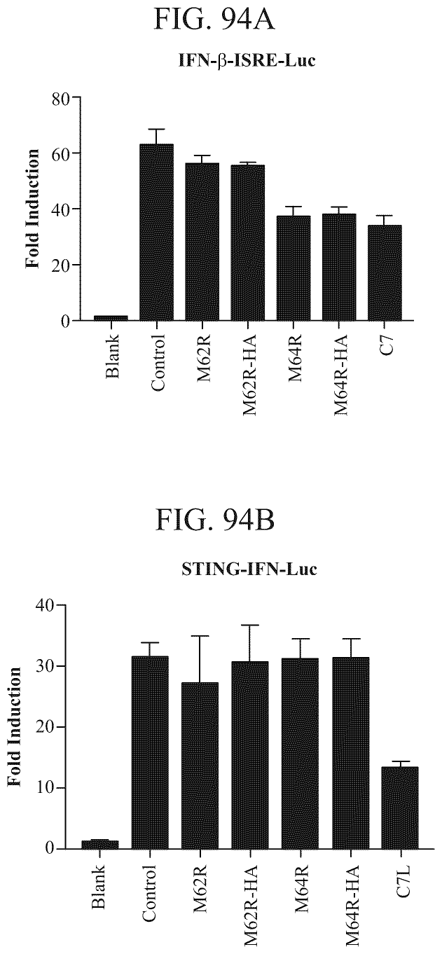

To determine whether myxoma M62 or M64 has similar inhibitory effect of C7 on IFNAR signaling, HEK293T cells were transfected with ISRE-firefly luciferase reporter, a control plasmid pRL-TK that expresses Renilla luciferase, myxoma M62R, Myxoma M62R-HA, Myxoma M64R, Myxoma M64R-HA, vaccinia C7L-expressing or control plasmid. 24 h post transfection, cells were treated with IFN-β for another 24 h before harvesting. Luciferase activities were measured. The results demonstrate that transient overexpression of myxoma M64 inhibits IFN-β-induced ISRE activation (FIG. 94A).

To determine whether myxoma M62 or M64 has similar inhibitory effect of C7 on STING-induced IFNB promoter activation, HEK293T cells were transfected with ISRE-firefly luciferase reporter, a control plasmid pRL-TK that expresses Renilla luciferase, and STING-expressing plasmid, together with either myxoma M62R, Myxoma M62R-HA, Myxoma M64R, Myxoma M64R-HA, vaccinia C7L-expressing, or control plasmid. The results demonstrate that transient overexpression of myxoma M64 or M62 fails to inhibit STING-induced IFNB promoter activation (FIG. 94B).

### Example 92: The Combination of IT Injection of the Engineered Poxviruses of the Present Technology with Systemic Delivery of any Combination of (i) One or More Immune Checkpoint Blocking Agents and/or One or More Immune System Stimulators; (ii) One or More Anti-Cancer Drugs; and (iii) an Immunomodulatory Drug (i.e., Fingolimod (FTY720)) is More Effective than IT Virus Alone in Treating a Solid Tumor

To test whether the combination with IT delivery of the engineered poxviruses of the present technology (e.g., MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4) and systemic delivery of any combination of: (i) one or more immune checkpoint blocking agents and/or one or more immune system stimulators; (ii) one or more anti-cancer drugs; and (iii) an immunomodulatory drug (i.e., fingolimod (FTY720)) had superior anti-tumor efficacy compared with IT virus alone against large established B16-F10 melanoma, 5×10⁵ cells are intradermally implanted into the right flanks of C57B/6 mice. Nine days after tumor implantation, when the tumors were 5 mm in diameter, they are treated with either: (a) IT PBS; (b) IT MVAΔE3L-OX40L, MVAΔC7L-OX40L, MVAΔC7L-hFlt3L-OX40L, MVAΔC7LΔE5R-hFlt3L-OX40L, MVAΔE5R-hFlt3L-OX40L, MVAΔE3LΔE5R-hFlt3L-OX40L, MVAΔE5R-hFlt3L-OX40L-ΔC11R, MVAΔE3LΔE5R-hFlt3L-OX40L-ΔC11R, VACVΔC7L-OX40L, VACVΔC7L-hFlt3L-OX40L, VACVΔE5R, VACV-TK⁻-anti-CTLA-4-ΔE5R-hFlt3L-OX40L, VACVΔB2R, VACVE3LΔ83NΔB2R, VACVΔE5RΔB2R, VACVE3LΔ83NΔE5RΔB2R, VACVE3LΔ83N-ΔTK-anti-CTLA-4-ΔE5R-hFlt3L-OX40L-IL-12, VACVE3LΔ83N-ΔTK-anti-CTLA-4-ΔE5R-hFlt3L-OX40L-IL-12-ΔB2R, MYXVΔM31R, MYXVΔM31R-hFlt3L-OX40L, MYXVΔM63R, MYXVΔM64R, MVAΔWR199, MVAΔE5R-hFlt3L-OX40L-ΔWR199, MVAΔE3LΔE5R-hFlt3L-mOX40LΔWR199-hIL-12, MVAΔE3LΔE5R-hFlt3L-mOX40LΔWR199-hIL-12ΔC11R, MVAΔE3LΔE5R-hFlt3L-mOX40LΔWR199-hIL-12ΔC11R-hIL-15/IL-15α, VACVΔE5R-IL-15/IL-15Rα, VACVΔE5R-IL-15/IL-15Rα-OX40L, VACVΔE3L83N-ΔTK-anti-huCTLA-4-ΔE5R-hFlt3L-hOX40L-hIL-12ΔB2RΔWR199ΔWR200, VACVΔE3L83N-ΔTK-anti-huCTLA-4-ΔE5R-hFlt3L-hOX40L-hIL-12ΔB2RΔWR199ΔWR200-hIL-15/IL-15Rα, VACVΔE3L83N-ΔTK-anti-huCTLA-4-ΔE5R-hFlt3L-hOX40L-hIL-12ΔB2RΔWR199ΔWR200ΔC11R, VACVΔE3L83N-ΔTK-anti-huCTLA-4-ΔE5R-hFlt3L-hOX40L-hIL-12ΔB2RΔWR199ΔWR200-hIL-15/IL-15RαΔC11R, MYXVΔM63RΔM64R, MYXVΔM62R, MYXVΔM62RΔM63RΔM64R, MYXVΔM31R, MYXVΔM62RΔM63RΔM64RΔM31R, MYXVΔM63RΔM64R-hFlt3L-OX40L, MYXVΔM62RΔM63RΔM64R-hFlt3L-OX40L, MYXVΔM62RΔM63RΔM64RΔM31R-hFlt3L-OX40L, MYXVΔM62RΔM63RΔM64RΔM31R-hFlt3L-OX40L-IL-12, MYXVΔM62RΔM63RΔM64RΔM31R-hFlt3L-OX40L-IL-12-IL-15/IL-15Rα, MYXVΔM62RΔM63RΔM64RΔM31R-hFlt3L-OX40L-IL-12-anti-CTLA-4, and/or MYXVΔM62RΔM63RΔM64RΔM31R-hFlt3L-OX40L-IL-12-IL-15/IL-15Rα-anti-CTLA-4 plus IP administration of (i) one or more immune checkpoint blocking agents and/or one or more immune system stimulators; (ii) one or more anti-cancer drugs; and (iii) an immunomodulatory drug (i.e., fingolimod (FTY720)); twice weekly. Tumor volumes are measured and mice survival is monitored. It is anticipated that the combined administration of one or more engineered poxviruses of the present technology and: (i) one or more immune checkpoint blocking agents and/or one or more immune system stimulators; (ii) one or more anti-cancer drugs; and/or (iii) an immunomodulatory drug (i.e., fingolimod (FTY720)) will result in enhanced anti-tumor effects as compared to the administration of the engineered poxvirus alone.

Accordingly, these results will show that the combined administration of engineered poxviruses of the present technology and (i) one or more immune checkpoint blocking agents and/or one or more immune system stimulators; (ii) one or more anti-cancer drugs; and/or (iii) an immunomodulatory drug (i.e., fingolimod (FTY720)), are useful in methods for treating solid tumors. It is further anticipated that the combined administration of engineered poxviruses of the present technology and (i) one or more immune checkpoint blocking agents and/or one or more immune system stimulators; (ii) one or more anti-cancer drugs; and/or (iii) an immunomodulatory drug (i.e., fingolimod (FTY720)) will produce synergistic effects in this regard as compared to the administration of engineered poxvirus alone.

### Example 93: Generation of Recombinant MVAΔE5R Expressing hFlt3L and mOX40L

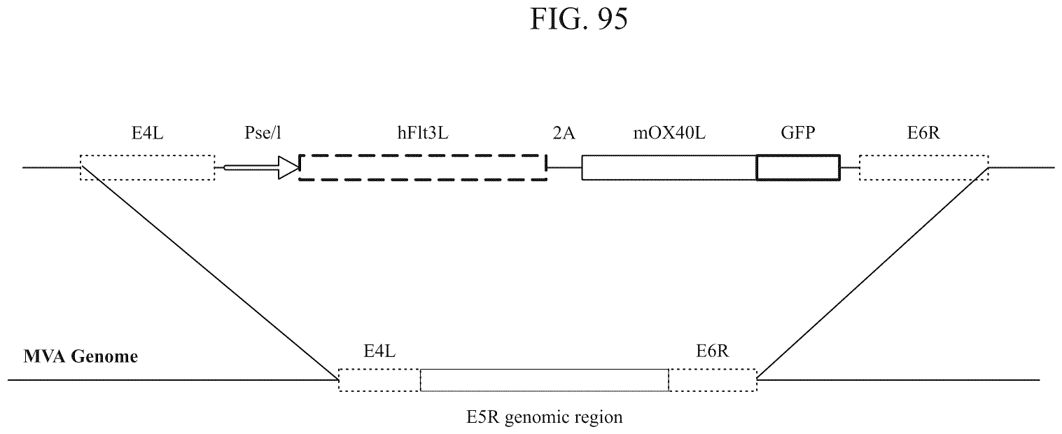

This example describes the generation of recombinant MVAΔE5R virus expressing hFlt3L and mOX40L. FIG. 95 shows a scheme of generating recombinant MVAΔE5R expressing hFlt3L and mOX40L through homologous recombination at the E4L and E6R loci of MVA genome. pUC57 vector is used to insert a single expression cassette designed to express both hFlt3L and mOX40L using the vaccinia viral synthetic early and late promoter (PsE/L). The coding sequence of the hFlt3L and mOX40L was separated by a furin cleavage site followed by a Pep2A sequence. Homologous recombination that occurred at the E4L and E6R loci results in the insertion of expression cassette for hFlt3L and mOX40L.

### Example 94: Expression of hFlt3L and mOX40L by Cells Infected with MVAΔE5R-hFlt3L-mOX40L

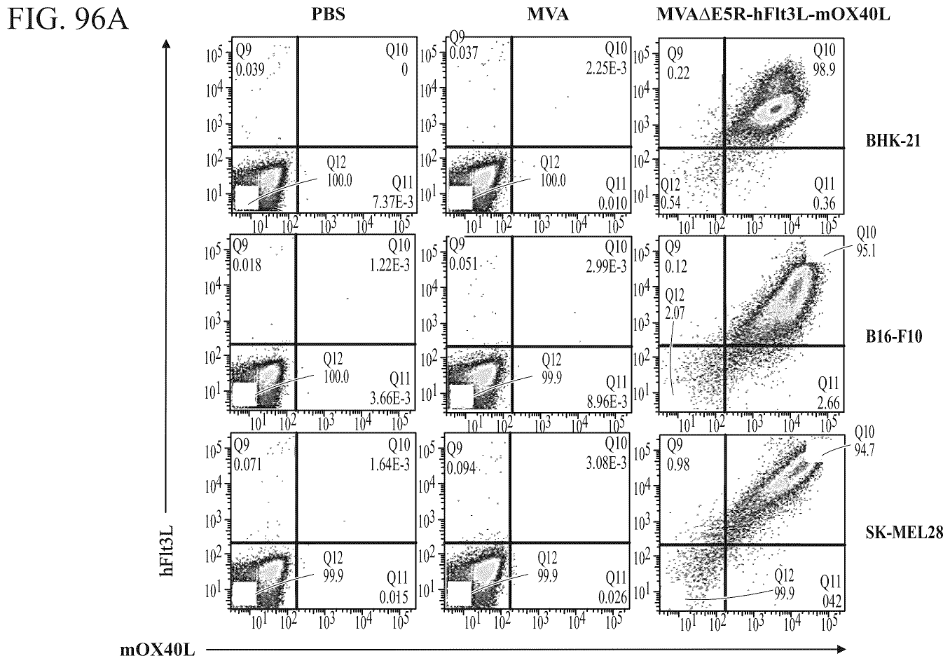

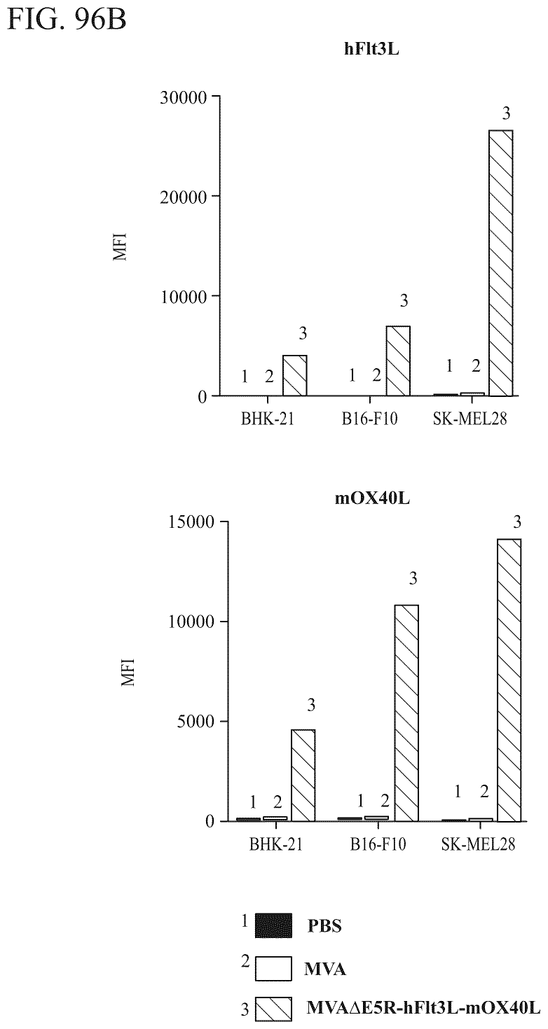

To test whether the recombinant MVAΔE5R-hFlt3L-mOX40L virus expresses both hFlt3L and mOX40L on the surface of infected cells, the following experiment were performed. BHK-21, murine B16-F10 melanoma cells and human SK-MEL28 melanoma cells were infected with either MVA or MVAΔE5R-hFlt3L-mOX40L at MOI 10. No virus mock infection control was included. 24 hours post infection, cells were harvested for surface staining with hFlt3L and mOX40L antibodies. Surface expression of hFlt3L and mOX40L was analyzed by FACS analysis. FIG. 96A are dot plots of surface expression of hFlt3L and mOX40L in BHK-21, B16-F10 and SK-MEL28 cells after infection. FIG. 96B are bar plots of mean fluorescence intensity (MFI) of hFlt3L and mOX40L expression in BHK-21, B16-F10 and SK-MEL28 cells after infection. The results show that MVAΔE5R-hFlt3L-mOX40L expressed mOX40L and hFlt3L efficiently in BHK-21, B16-F10 and SK-MEL28 cells at 24 h post infection. Given that MVAΔE5R-hFlt3L-mOX40L does not replicate in B16-F10 or SK-MEL28, the expression of hFlt3L and mOX40L on the infected tumor cells was robust.

### Example 95: IT Injection of MVAΔE5R-hFlt3L-mOX40L Induces Stronger Systemic Antitumor T-Cell Immunity Compared with MVA, MVAΔE5R or Heat-iMVA

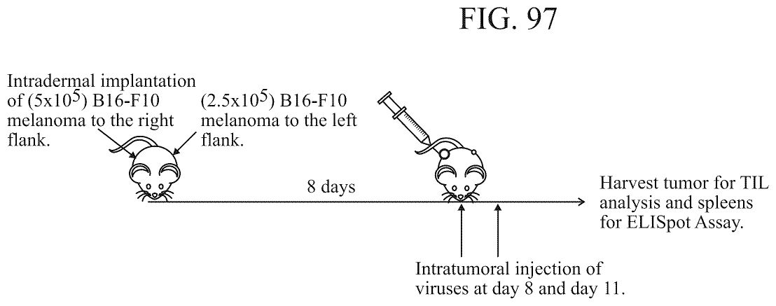

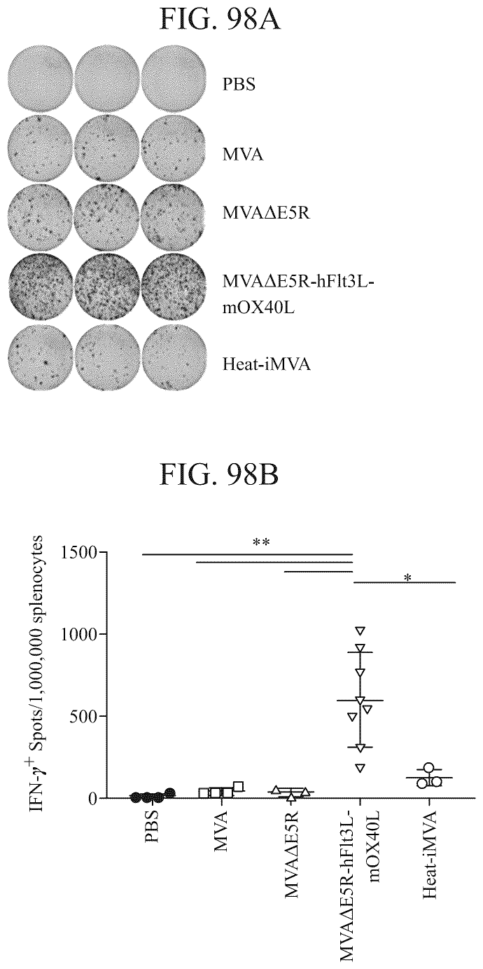

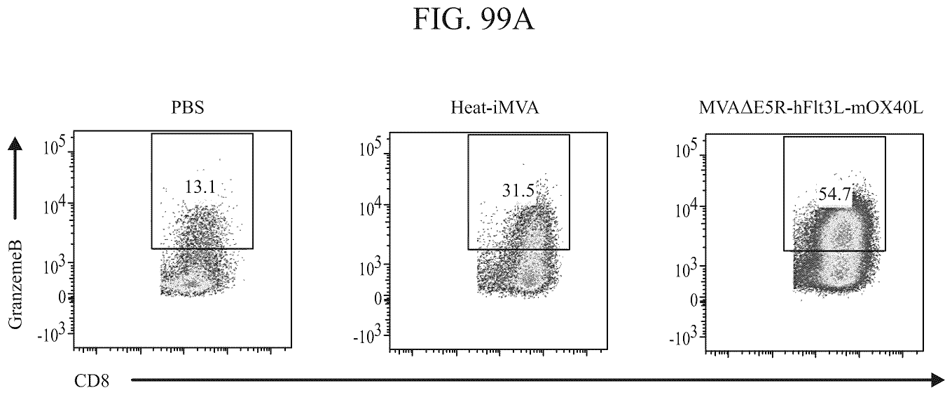

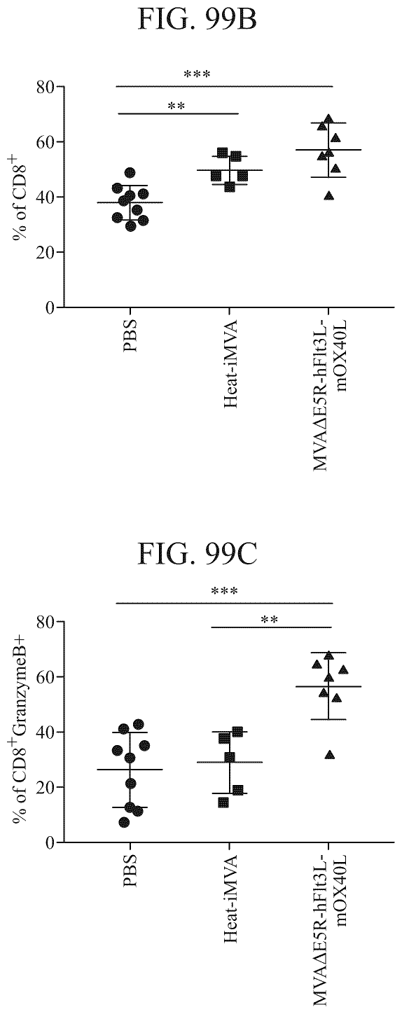

ELISpot was performed to assess the generation of antitumor specific T cells in the spleens of mice treated with MVA, MVAΔE5R, MVAΔE5R-hFlt3L-mOX40L or Heat-iMVA. Briefly, B16-F10 melanoma cells were implanted intradermally into the shaved skin on the right (5×10⁵ cells) and left (2.5×10⁵ cells) flanks of a C57BL/6J mouse. 8 days post implantation, the larger tumors on the right flank were injected twice per week with 4×10⁷ pfu of MVA, MVAΔE5R or MVAΔE5R-hFlt3L-mOX40L, or with an equivalent amount of Heat-iMVA. Spleens were harvested for ELISpot analysis (FIG. 97). 1,000,000 splenocytes were cultured with 1.5×10⁵ irradiated B16-F10 cells overnight at 37° C. in anti-IFN-γ-coated BD ELISpot plate microwells. Splenocytes were stimulated with B16-F10 cells irradiated with an γ-irradiator and IFN-γ secretion was detected with an anti-IFN-γ antibody. FIGS. 98A-98B shows representative images of IFN-γ⁺ spots per 1,000,000 splenocytes from individual mouse treated with either PBS, MVAΔE5R, MVAΔE5R-hFlt3L-mOX40L, or Heat-iMVA. FIGS. 99A-99C shows the numbers of IFN-γ⁺ spots per 1,000,000 splenocytes from individual mouse in each group treated with either PBS, MVAΔE5R, MVAΔE5R-hFlt3L-mOX40L, or Heat-iMVA. These results demonstrate that IT injection of MVAΔE5R-hFlt3L-mOX40L is more effective than MVA, MVAΔE5R or Heat-iMVA in generating antitumor T cells in treated mice in a murine B16-F10 melanoma bilateral implantation model.

### Example 96: Intratumoral (IT) Injection of MVAΔE5R-hFlt3L-mOX40L Leads to Activation of CD8⁺ and CD4⁺ T Cells in Both Injected and Non-Injected Distant Tumors in B16-F10 Bilateral Tumor Implantation Model

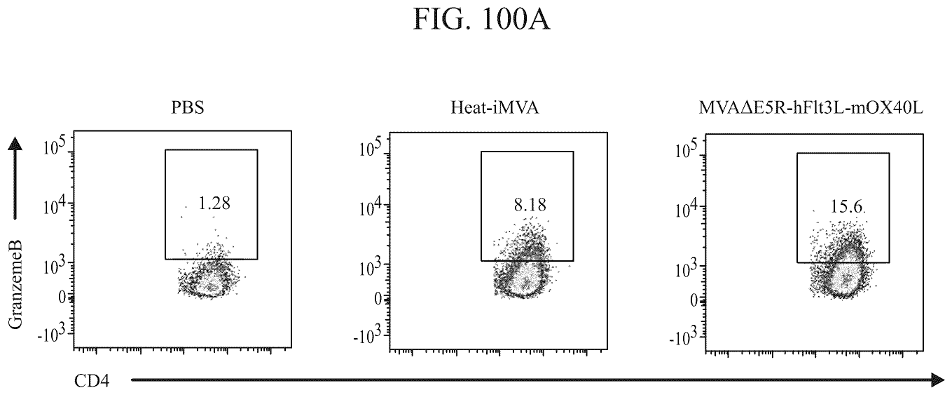

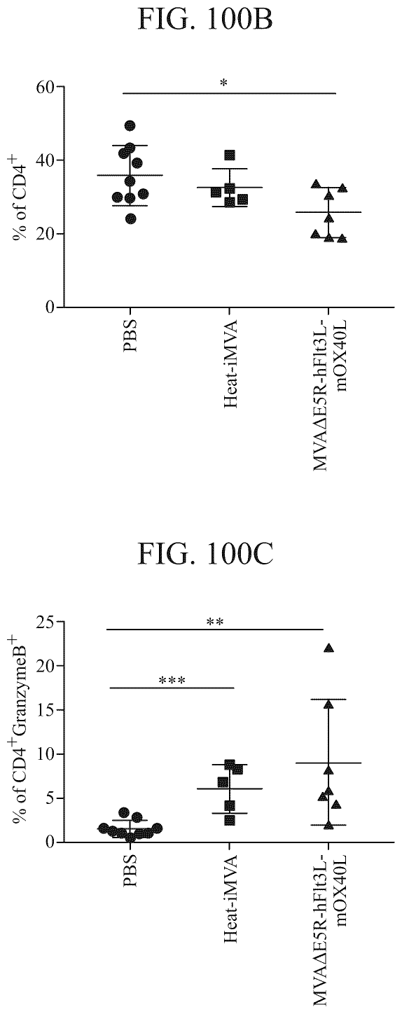

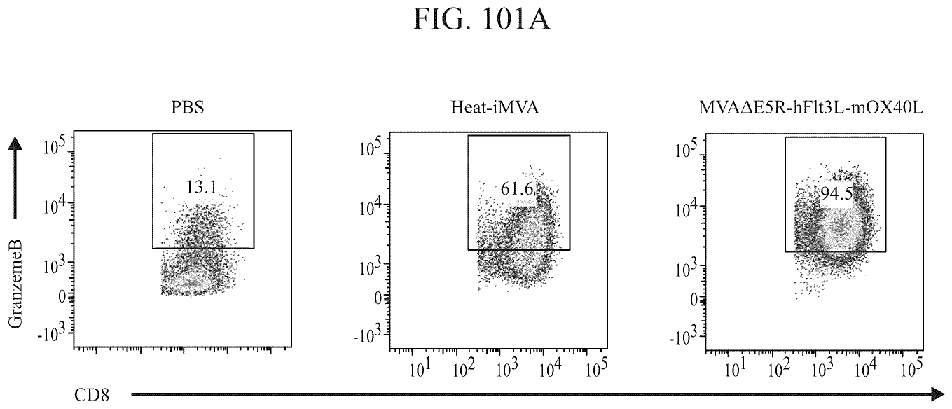

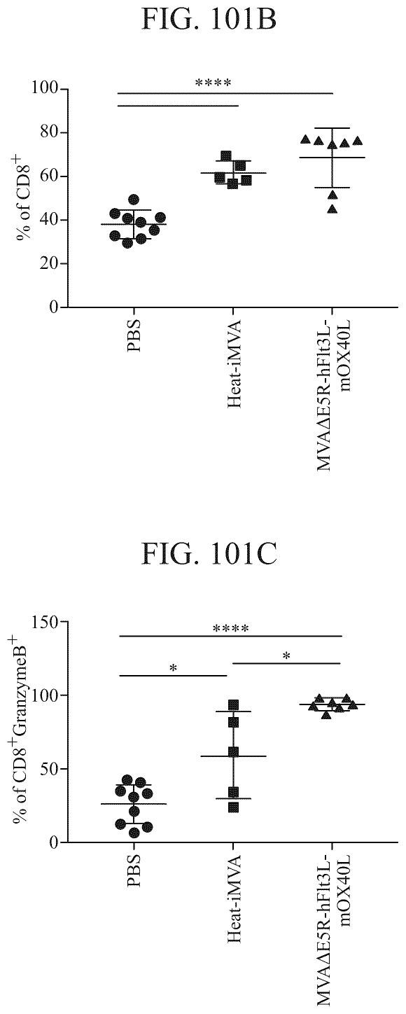

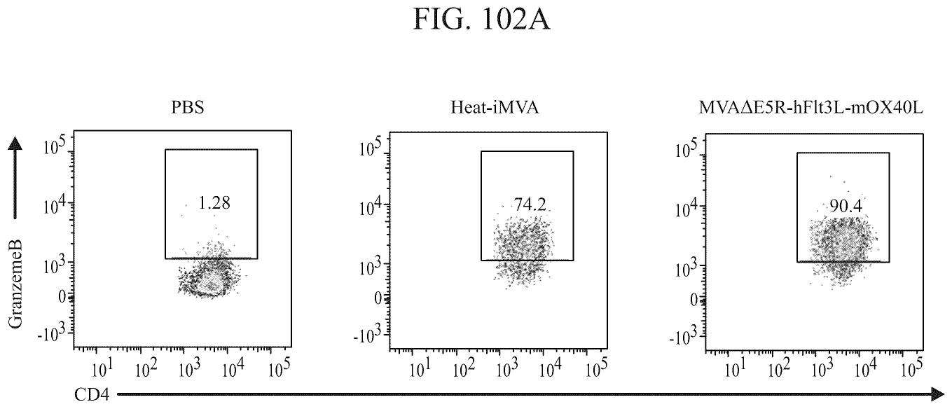

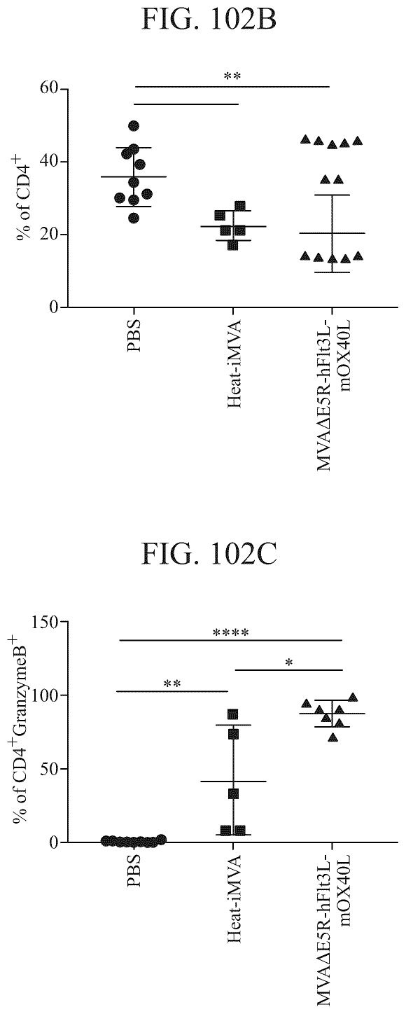

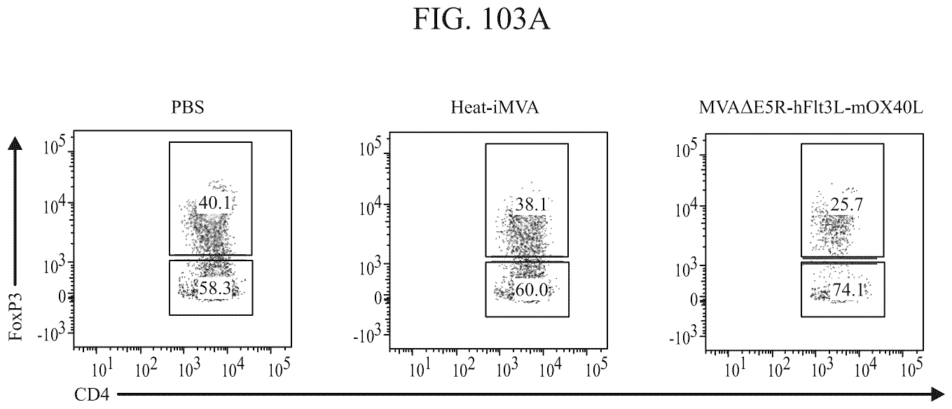

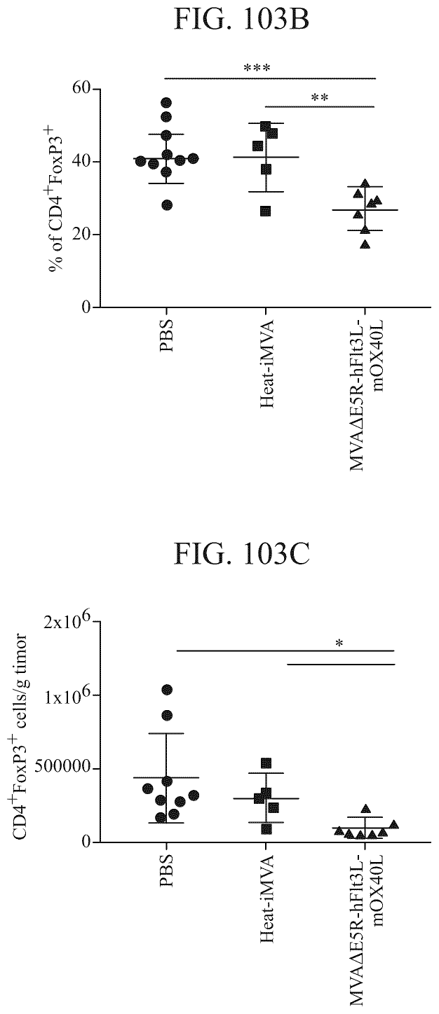

To assess whether IT MVAΔE5R-hFlt3L-mOX40L results in the generation of local and systemic antitumor immunity, a bilateral B16-F10 tumor implantation model was used as described in Example 3. Two days after the second injection, tumors were harvested and cells were processed for surface labeling with anti-CD3, CD45, CD4, and CD8 antibodies, and also for intracellular Granzyme B staining. The live immune cell infiltrates in the tumors were analyzed by FACS. IT MVAΔE5R-hFlt3L-mOX40L resulted in higher percentage of total CD8⁺ T cells as well as Granzyme B⁺ CD8⁺ T cells in the non-injected tumors compared with Heat-iMVA (FIGS. 100A-100C). Remarkably, IT MVAΔE5R-hFlt3L-mOX40L resulted in higher percentage of total CD4⁺ T cells as well as Granzyme B⁺ CD4⁺ T cells in the non-injected tumors compared with Heat-iMVA (FIGS. 101A-101C). In addition, IT MVAΔE5R-hFlt3L-mOX40L also resulted in higher percentage of total CD8⁺ T cells and Granzyme B⁺ CD8⁺ T cells in the injected tumors compared with Heat-iMVA (FIGS. 102A-102C). In addition, IT MVAΔE5R-hFlt3L-mOX40L induced higher percentage of Granzyme B⁺ CD4⁺ T cells in the injected tumors compared with Heat-iMVA (FIGS. 103A-103C). These results demonstrate that IT MVAΔE5R-hFlt3L-mOX40L is more effective than Heat-iMVA in inducing cytotoxic CD8⁺ T cells and CD4⁺ T cells within both injected and non-injected tumors, and demonstrate that these viral constructs are useful in methods for treating solid tumors.

### Example 97: Intratumoral (IT) Injection of MVAΔE5R-hFlt3L-mOX40L Leads to Reduction of Regulatory T Cells in Injected Distant Tumors in B16-F10 Bilateral Tumor Implantation Model

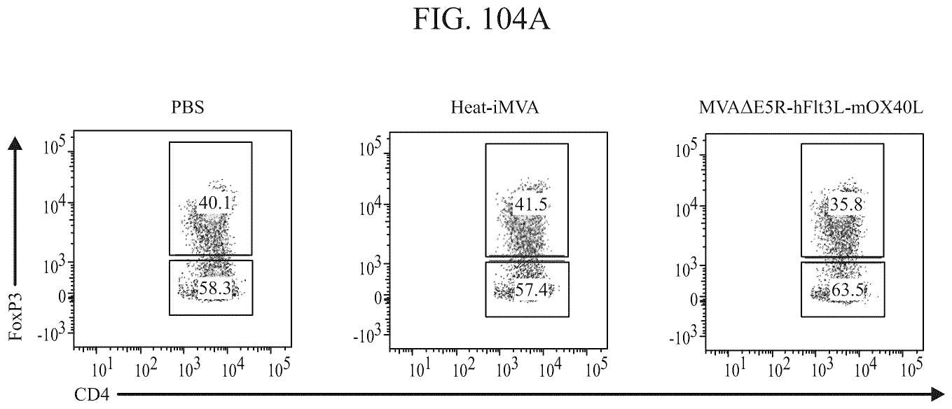

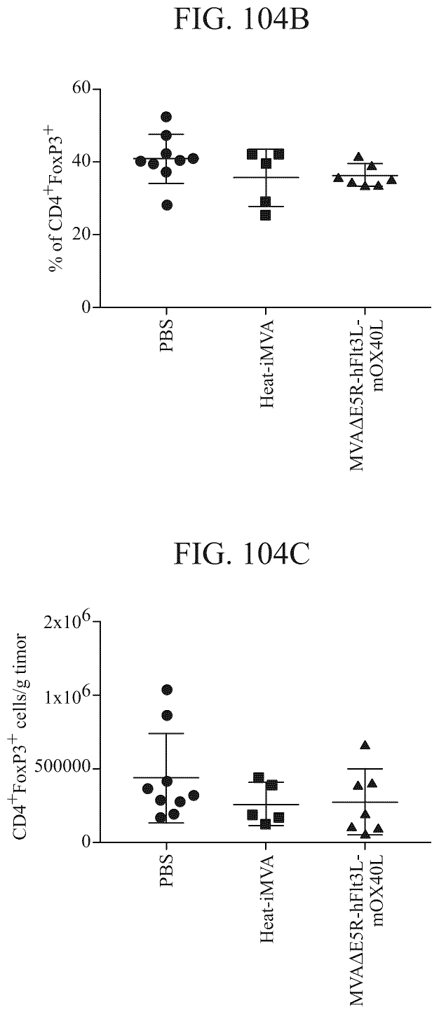

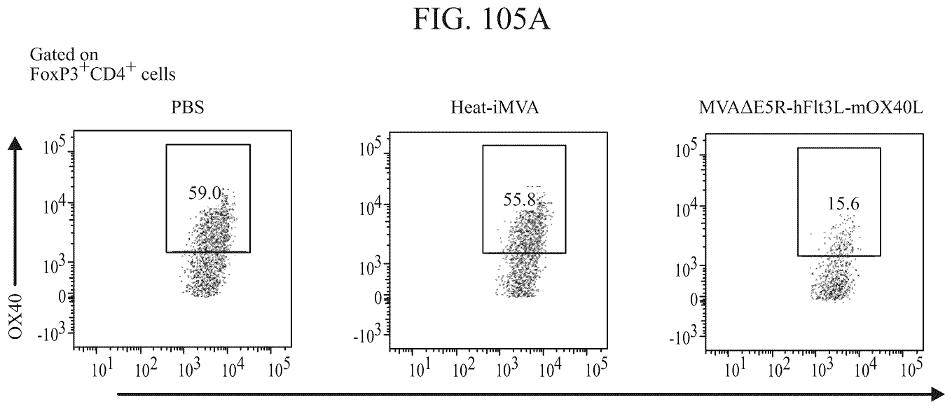

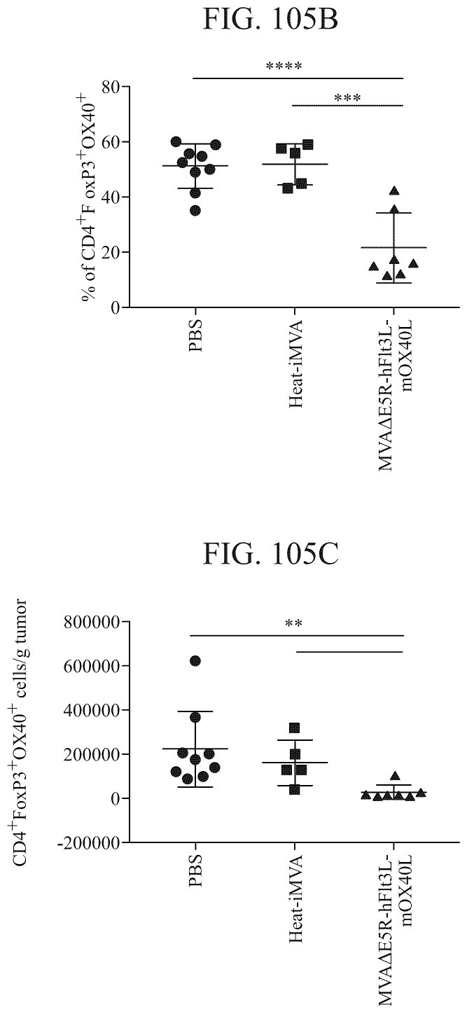

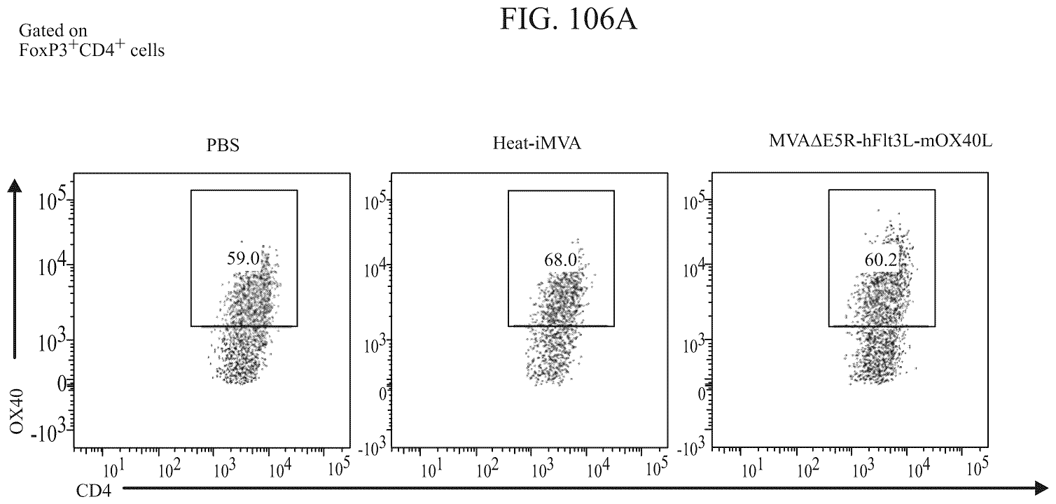

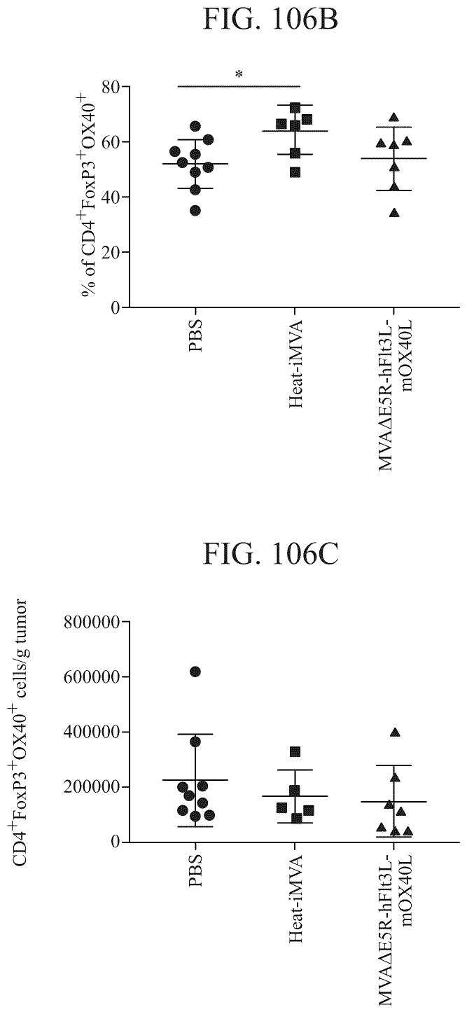

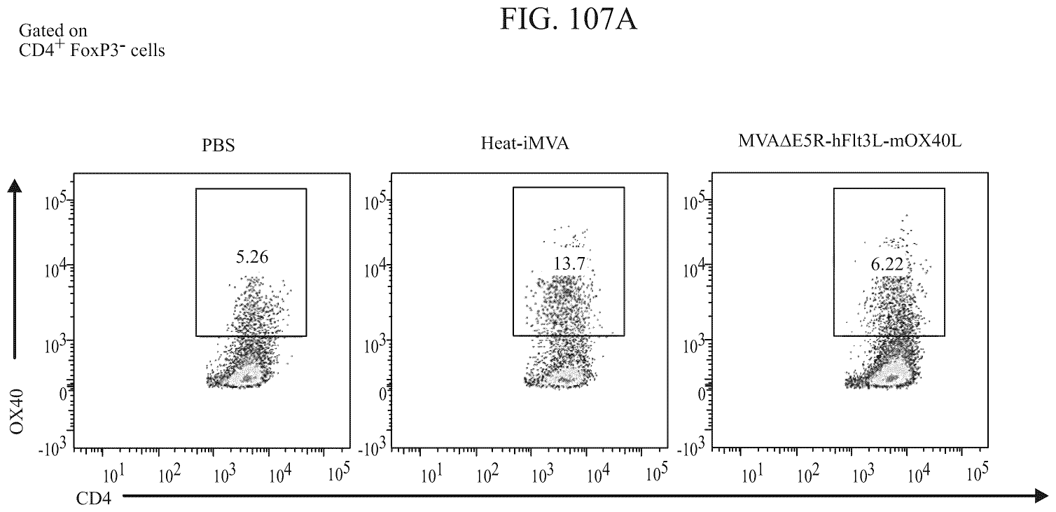

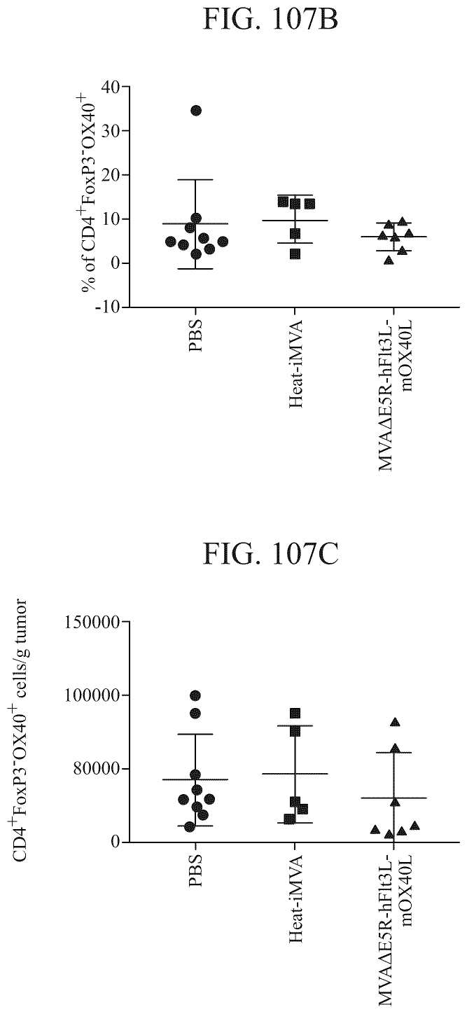

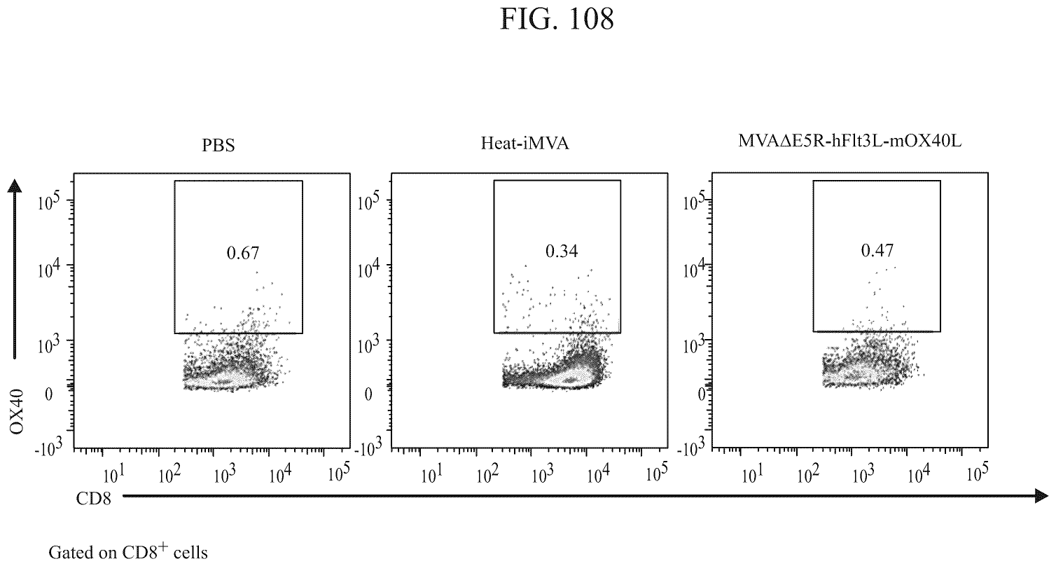

To assess whether IT MVAΔE5R-hFlt3L-mOX40L affected tumor infiltrating regulatory T cells, a bilateral B16-F10 tumor implantation model was used as described in Example 95. Two days after the second injection, tumors were harvested and cells were processed for surface labeling with anti-CD3, CD45, CD4, CD8 and OX40 antibodies, and also for intracellular FoxP3 staining. The live immune cell infiltrates in the tumors were analyzed by FACS. IT MVAΔE5R-hFlt3L-mOX40L resulted in reduced percentage and absolute number of CD4⁺FoxP3⁺ T cells in the injected tumors (FIGS. 103A-103C). There was no significant difference of the percentage and absolute number of Tregs in the non-injected tumors among PBS, Heat-iMVA and IT MVAΔE5R-hFlt3L-mOX40L treated groups (FIGS. 104A-104C). OX40, the receptor of OX40L was highly expressed on CD4⁺FoxP3⁺ T cells (FIGS. 105A-105C). IT MVAΔE5R-hFlt3L-mOX40L resulted in reduced percentage and absolute number OX40⁺CD4⁺FoxP3⁺ T cells in the injected tumors (FIGS. 105A-105C), which was not observed in non-injected tumors (FIGS. 106A-106C). OX40 was not highly expressed in CD4⁺FoxP3⁻ T cells (FIGS. 107A-107C) and CD8⁺ T cells (FIG. 108). These results demonstrate that IT injection of MVAΔE5R-hFlt3L-mOX40L reduces OX40⁺CD4⁺FoxP3⁺ T cells in the injected tumors, and is useful in methods for treating solid tumors.

### Example 98: Reduction of Regulatory T Cells (Tregs) in Injected Distant Tumors by Intratumoral (IT) Delivery of MVAΔE5R-hFlt3L-mOX40L is Dependent on OX40L Expression by MVAΔE5R-hFlt3L-mOX40L

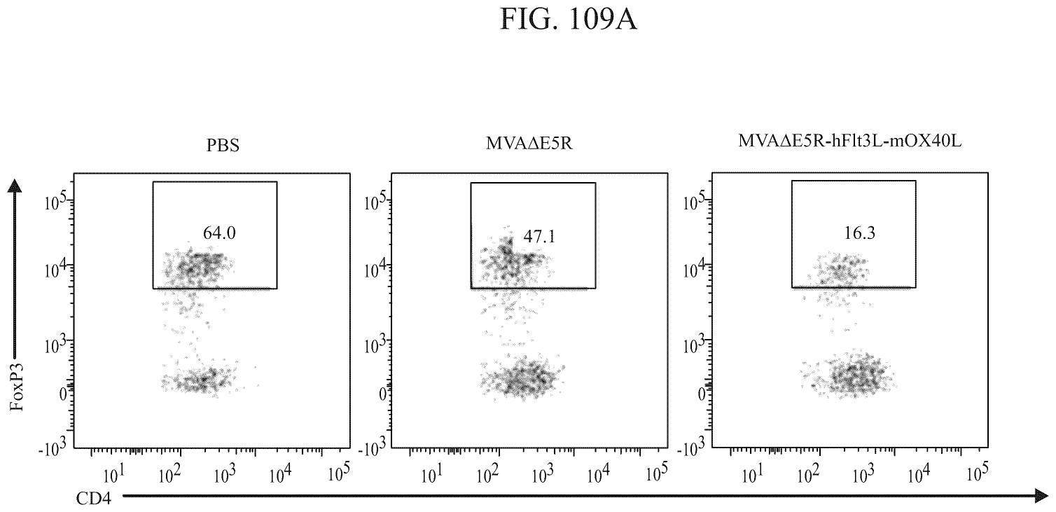

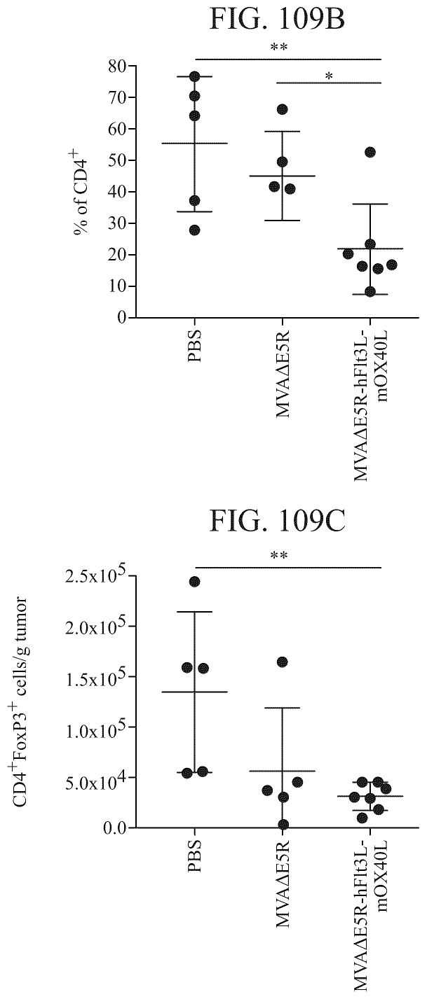

To assess whether the reduction of Tregs by IT injection of MVAΔE5R-hFlt3L-mOX40L was dependent on mOX40L expression, a bilateral B16-F10 tumor implantation model was used and compared the efficiency of reduction by IT delivery of MVAΔE5R-hFlt3L-mOX40L vs. MVAΔE5R. Briefly, B16-F10 melanoma cells were implanted intradermally into the shaved skin on the right (5×10⁵ cells) and left (2.5×10⁵ cells) flanks of a wild C57BL/6J. Eight days post implantation, the larger tumors on the right flank were injected twice per week with 4×10⁷ pfu of MVAΔE5R or MVAΔE5R-hFlt3L-mOX40L, or PBS. Two days post second injection, tumors were harvested and cells were processed for surface labeling with anti-CD3, CD45, CD4 and CD8 antibodies, and also for intracellular FoxP3 staining. The live immune cell infiltrates in the tumors were analyzed by FACS. In the injected tumors, IT injection of MVAΔE5R-hFlt3L-mOX40L resulted in significantly reduced percentage and absolute numbers of CD4⁺FoxP3⁺ T cells in the injected tumors compared with PBS (FIGS. 109A-109C). By contrast, IT injection of MVAΔE5R did not significantly affect the percentage of CD4⁺FoxP3⁺ T cells (FIGS. 109A-109C). These results demonstrate that depletion of Tregs in in the injected tumors by IT injection of MVAΔE5R-hFlt3L-mOX40L is dependent on OX40L expression by the recombinant virus.

### Example 99: Intratumoral (IT) Injection of MVAΔE5R-hFlt3L-mOX40L Leads to Stronger Activation of CD8⁺ and CD4⁺ T Cells in the Injected Tumors in OX40^{-/-} Compared with WT Mice in B16-F10 Bilateral Tumor Implantation Model

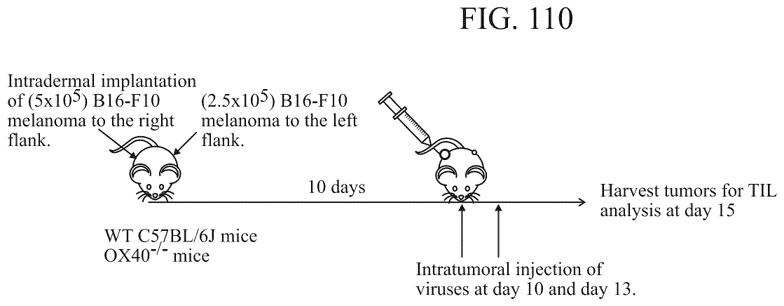

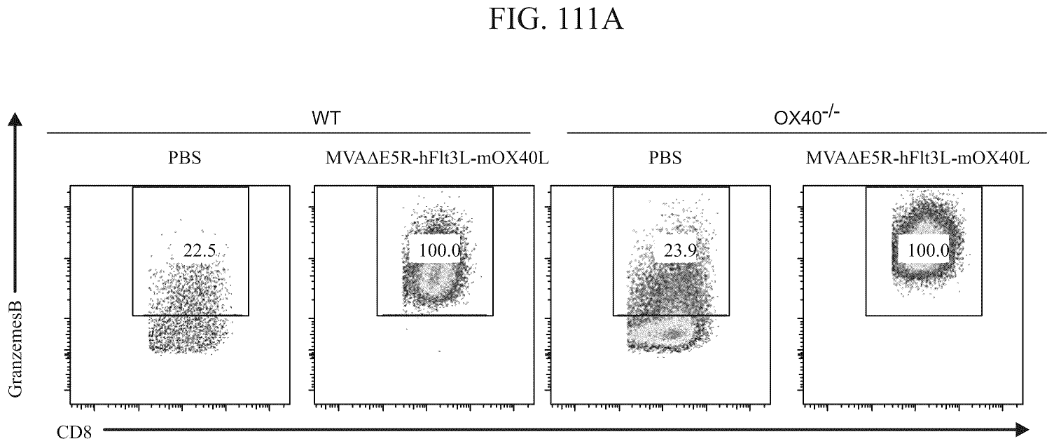

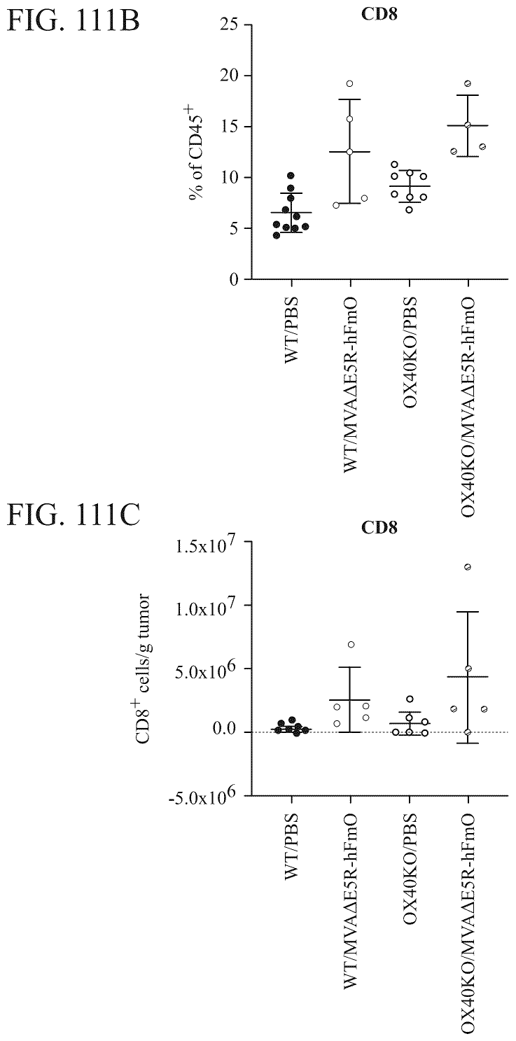

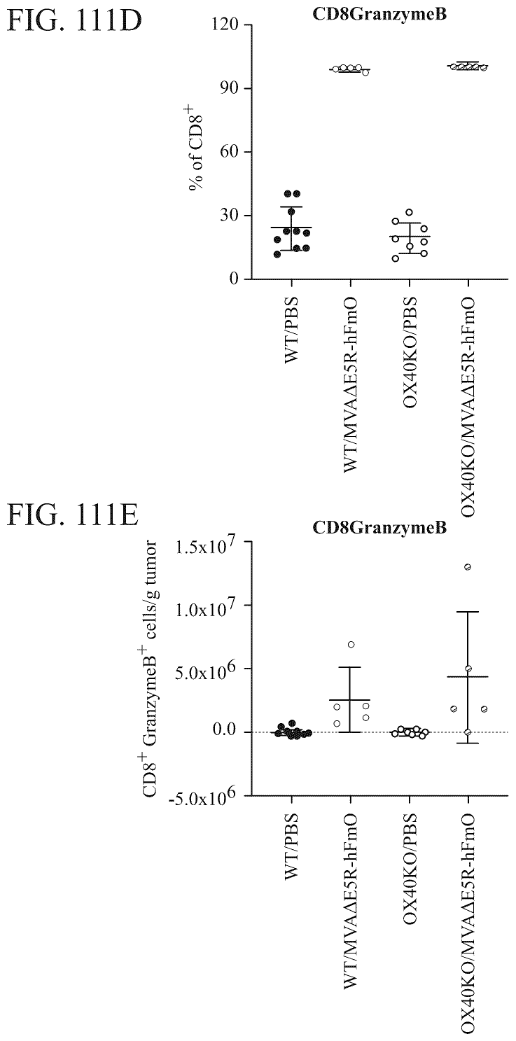

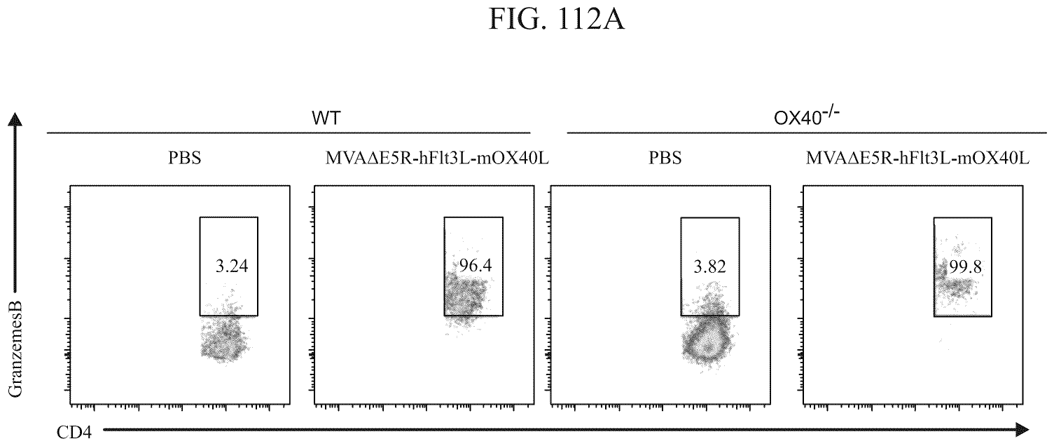

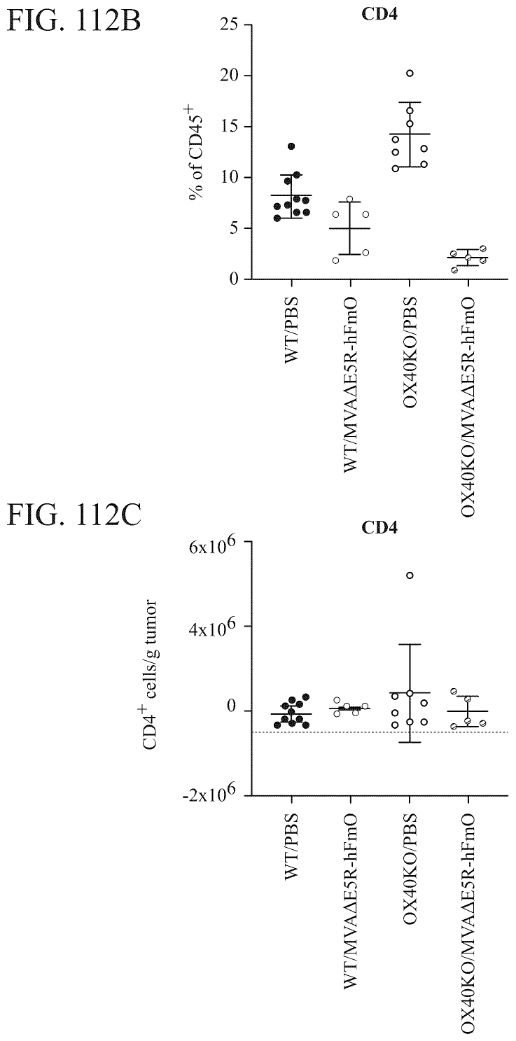

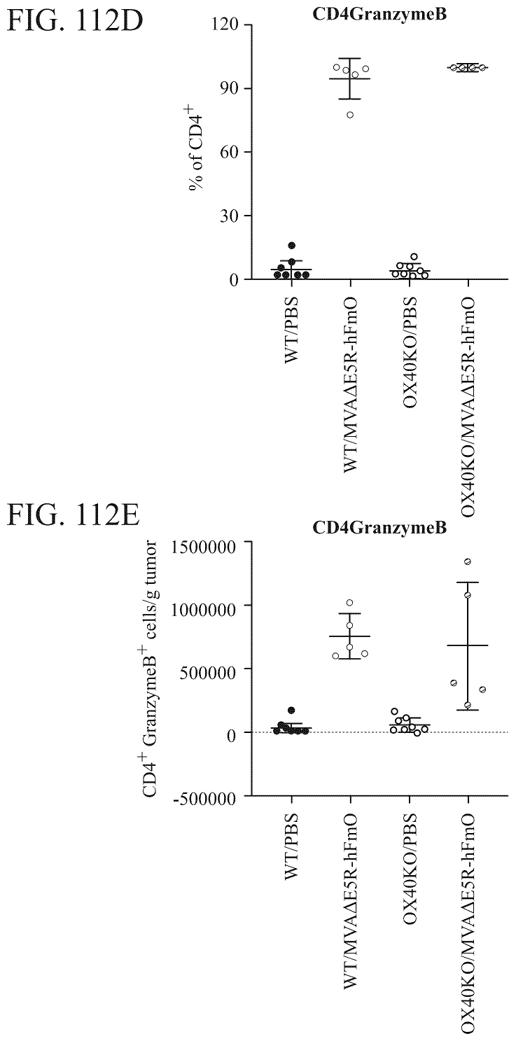

To compare the immune responses induced by IT injection of MVAΔE5R-hFlt3L-mOX40L in WT and OX40^{-/-} mice, a bilateral B16-F10 tumor implantation model was used. Briefly, B16-F10 melanoma cells were implanted intradermally into the shaved skin on the right (5×10⁵ cells) and left (2.5×10⁵ cells) flanks of wild-type C57BL/6J or OX40^{-/-} mice. Ten days post implantation, the larger tumors on the right flank were injected twice per week with 4×10⁷ pfu of MVAΔE5R or MVAΔE5R-hFlt3L-mOX40L or PBS. Two days post second injection, tumors were harvested and cells were processed for surface labeling with anti-CD3, CD45, CD4, CD8 and OX40 antibodies, and also for intracellular Granzyme B, Ki67 and FoxP3 staining. The live immune cell infiltrates in the tumors were analyzed by FACS (FIG. 110). In both wild-type and OX40^{-/-} mice, IT injection of MVAΔE5R-hFlt3L-mOX40L resulted in higher percentage and numbers of total CD8⁺ T cells and CD8⁺Granzyme B⁺ T cells in the injected tumors compared with PBS group (FIGS. 111A-111E). IT injection of MVAΔE5R-hFlt3L-mOX40L also resulted in higher percentage and numbers of total CD4⁺ T cells and CD4⁺Granzyme B⁺ T cells in the injected tumors from both wild-type and OX40^{-/-} mice (FIGS. 112A-112E). The Granzyme B expressions in CD8⁺ and CD4⁺ T cells in MVAΔE5R-hFlt3L-mOX40L-injected tumors were higher in OX40^{-/-} mice compared with WT mice (FIGS. 111A-112E). These results suggest that OX40 expression might be related in immune suppression in the tumors. Since tumor-infiltrating Tregs express high levels of OX40, without being bound by theory, it is postulated that OX40 expression on regulatory T cells might be important for their immune-suppressive function.

### Example 100: Intratumoral (IT) Injection of MVAΔE5R-hFlt3L-mOX40L Reduces Regulatory T Cells in the Injected Tumors from Wild-Type Mice but not OX40^{-/-} Mice in B16-F10 Bilateral Tumor Implantation Model

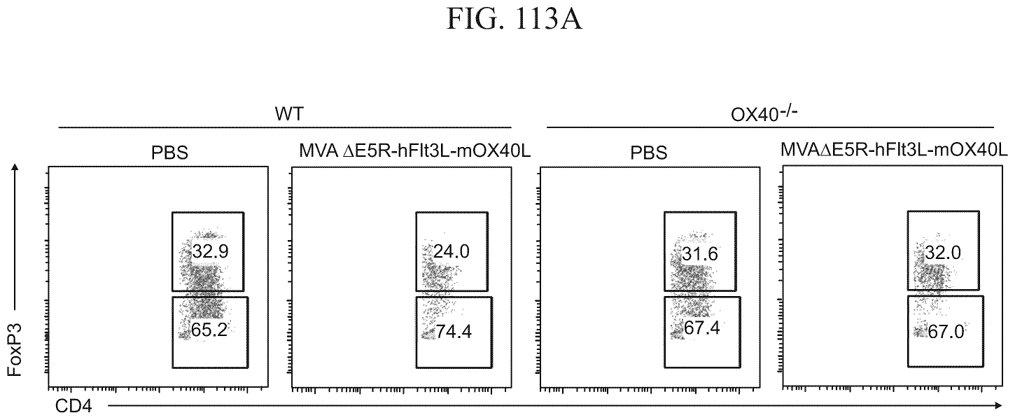

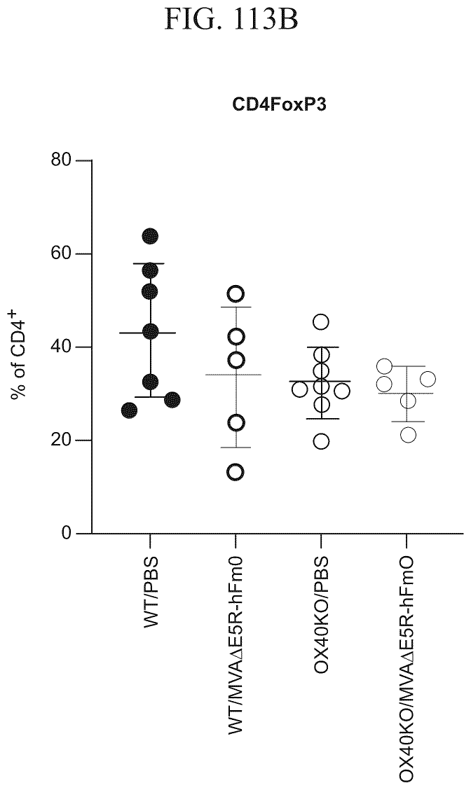

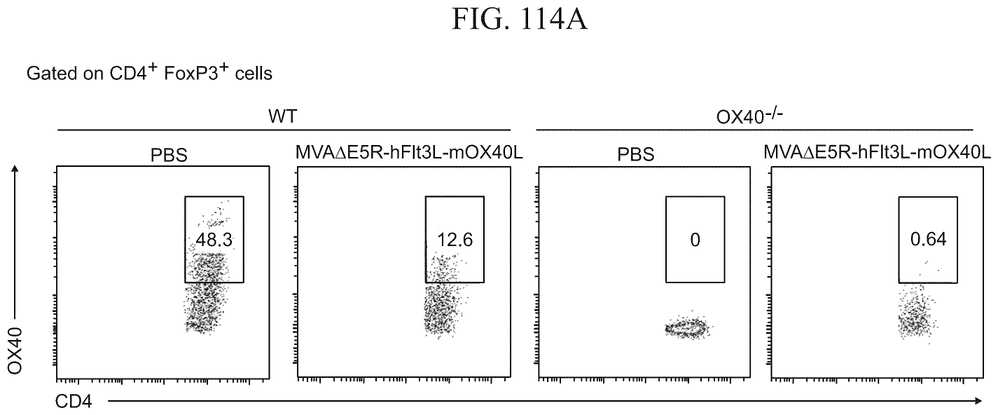

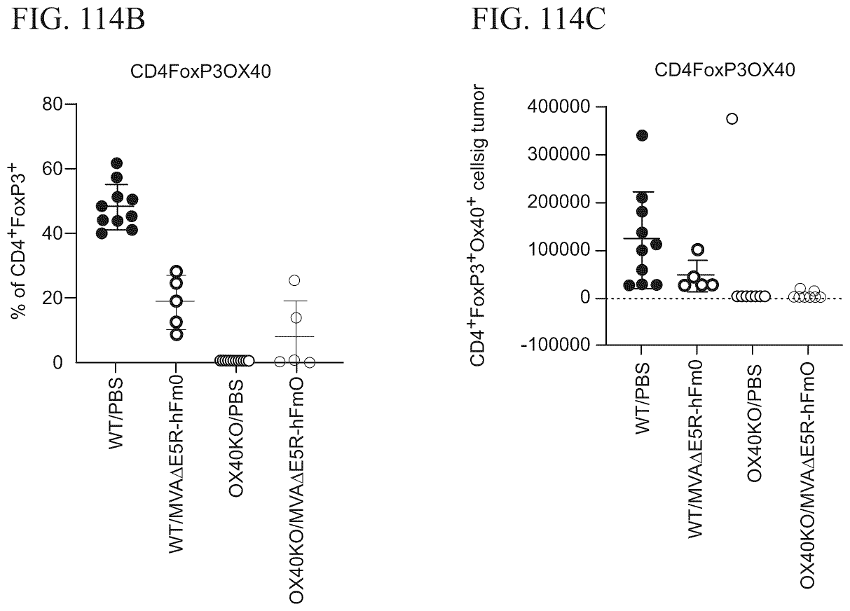

To test whether the reduction of regulatory T cells by IT injection of MVAΔE5R-hFlt3L-mOX40L is due to OX40L-OX40 interaction, the percentages of CD4⁺FoxP3⁺ T cells out of CD4⁺ T cells were compared in the injected tumors from wild-type mice treated with PBS or MVAΔE5R-hFlt3L-mOX40L vs. OX40^{-/-} mice treated with PBS or MVAΔE5R-hFlt3L-mOX40L. In WT mice, the percentages of CD4⁺FoxP3⁺ T cells out of CD4⁺ T cells were reduced in MVAΔE5R-hFlt3L-mOX40L-treated tumors compared with PBS-treated tumors. By contrast, in OX40^{-/-} mice, the percentages of CD4⁺FoxP3⁺ T cells out of CD4⁺ T cells were similar in PBS and MVAΔE5R-hFlt3L-mOX40L group (FIGS. 113A-113B). In WT mice, the percentages of OX40⁺FoxP3⁺CD4⁺ cells were reduced from 48% in the PBS-treated tumors to 19% in MVAΔE5R-hFlt3L-mOX40L-treated tumors (FIGS. 114A-114C). The absolute numbers of OX40⁺FoxP3⁺CD4⁺ cells per gram of tumor were reduced from—in the PBS-treated tumors to—in MVAΔE5R-hFlt3L-mOX40L-treated tumors (FIG. 114B). As expected, OX40 expression on FoxP3⁺CD4⁺ cells were absent (FIG. 114C). These results indicate that MVAΔE5R-hFlt3L-mOX40L-induced reduction of Tregs in injected tumors is likely dependent on the expression of OX40 on Tregs.

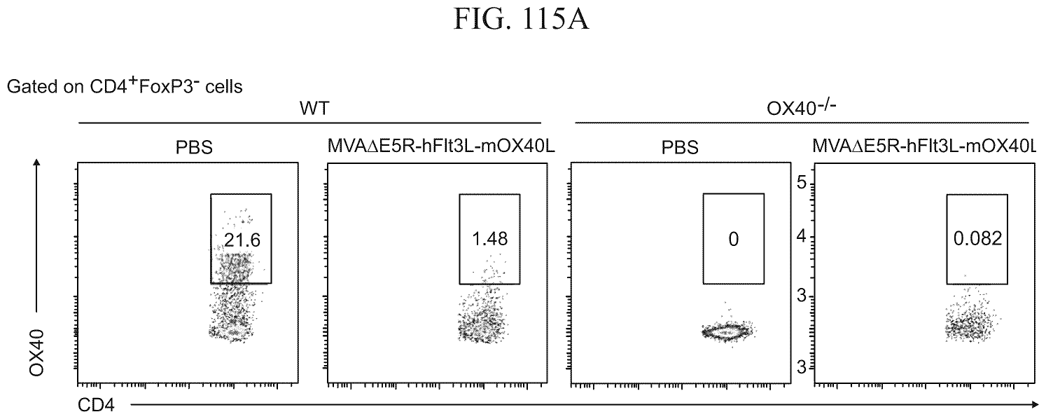

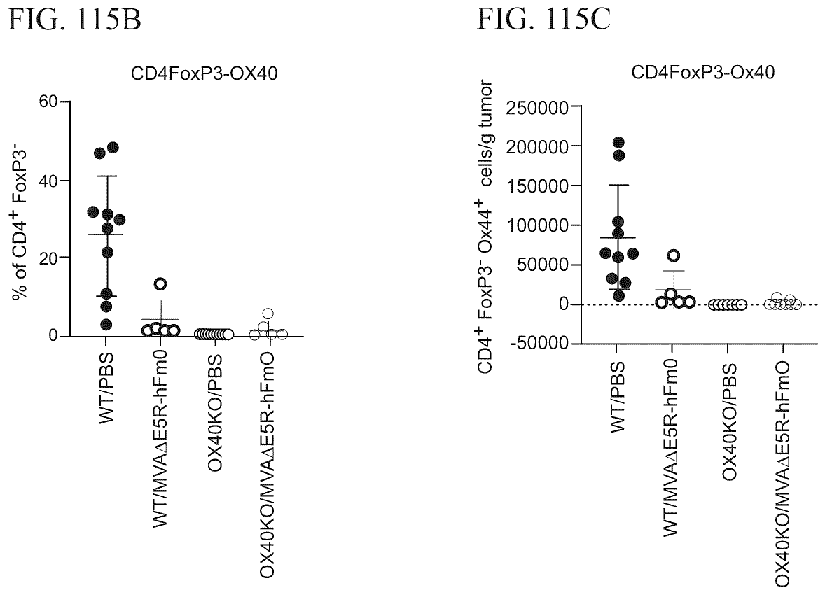

The expression of OX40 on FoxP3-CD4⁺ was also reduced in MVAΔE5R-hFlt3L-mOX40L-treated tumors compared with PBS-treated tumors in WT mice (FIGS. 115A-115C). Without wishing to be bound by theory, it is possible that OX40L/OX40 interaction might lead to failure to detect OX40 expression on these cells.

### Example 101: Intratumoral (IT) Injection of MVAΔE5R-hFlt3L-mOX40L Results in Stronger Activation and Proliferation of CD8⁺ and CD4⁺ T Cells in the Non-Injected Tumors from OX40^{-/-} Mice Compared with WT Mice

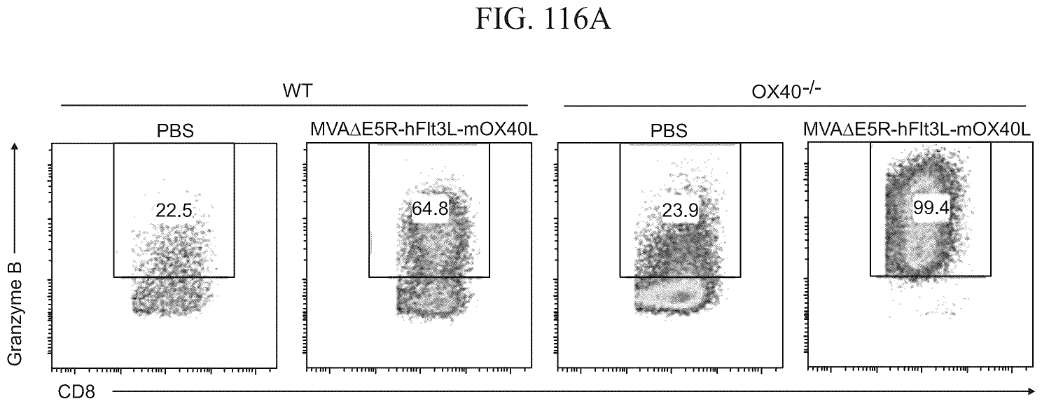

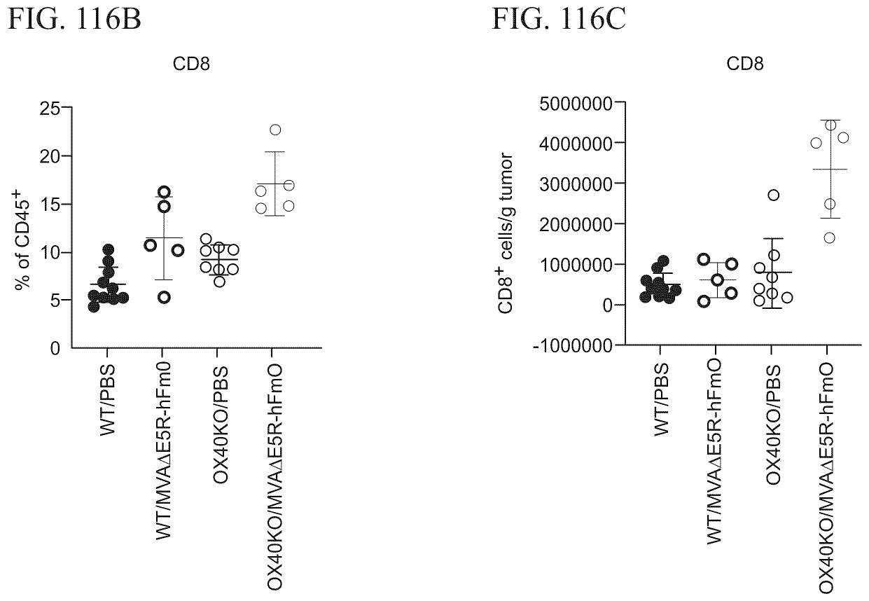

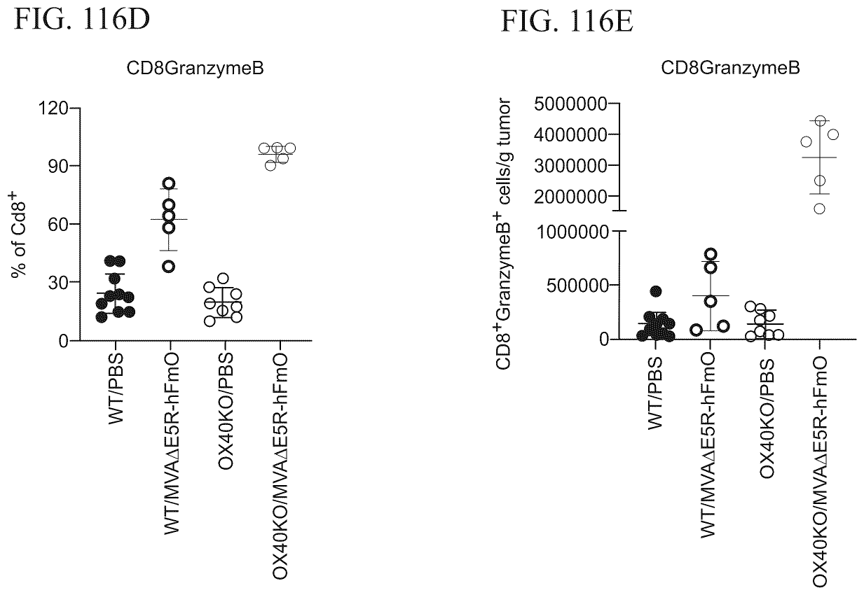

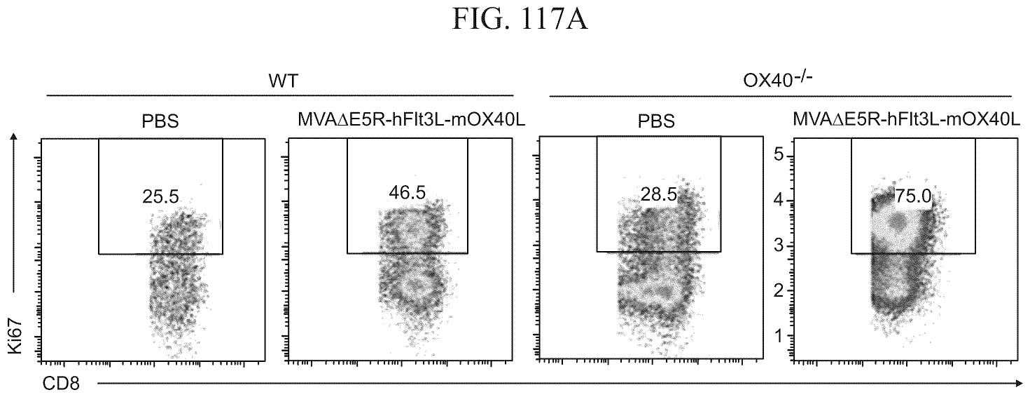

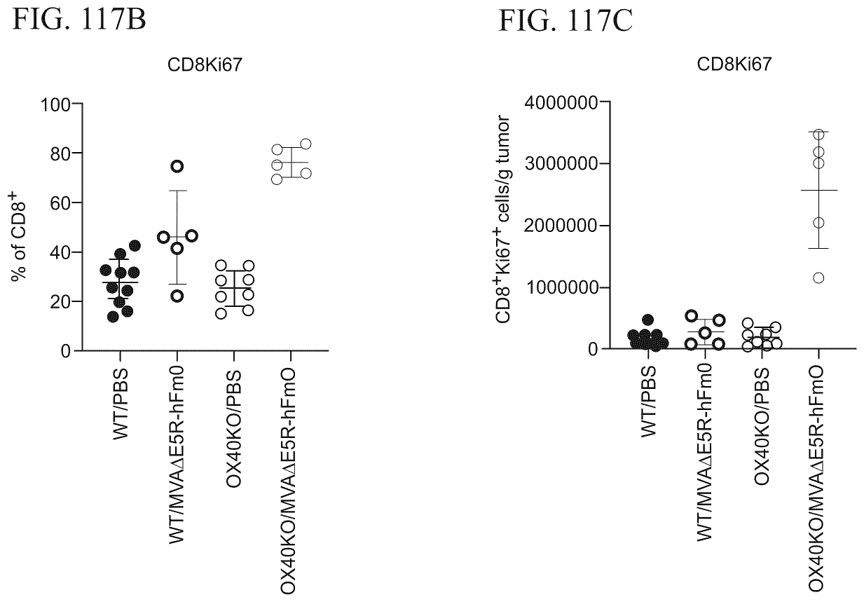

To test whether IT MVAΔE5R-hFlt3L-mOX40L results in immune responses in the non-injected tumors, the non-injected tumors were harvested 2 days after second injection with either MVAΔE5R-hFlt3L-mOX40L or PBS. FACS analysis was performed to evaluate Granzyme B (an activation marker) and Ki67 (a proliferation marker) expression on CD8⁺ and CD4⁺ T cells. IT MVAΔE5R-hFlt3L-mOX40L results in the increase of percentages of CD8⁺ cells out of CD45⁺ cells as well in the increase of percentages of Granzyme B⁺CD8⁺ cells out of CD8⁺ cells compared with those treated with PBS (FIGS. 116A-116E). In addition, IT MVAΔE5R-hFlt3L-mOX40L results in the increase of Ki67⁺CD8⁺ cells out of CD8⁺ T cells compared with those treated with PBS (FIGS. 117A-117C). In the OX40^{-/-} mice, IT MVAΔE5R-hFlt3L-mOX40L elicited stronger activation and proliferation responses on CD8⁺ T cells in the non-injected tumors compared with those in WT mice (FIGS. 116A-117C).

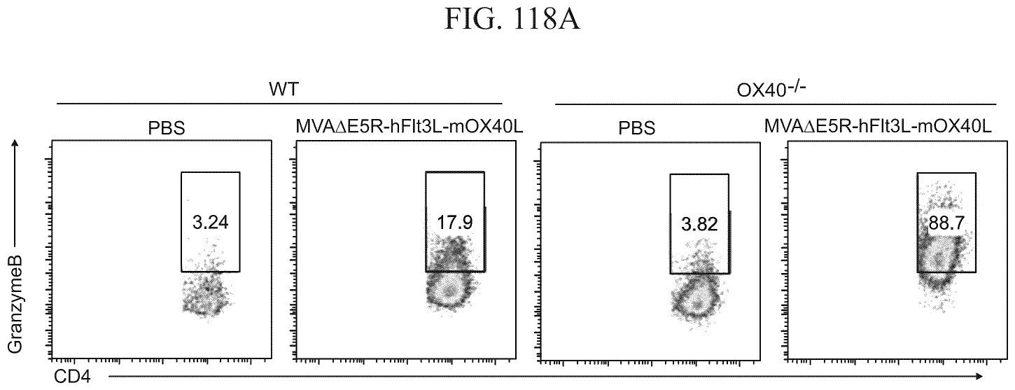

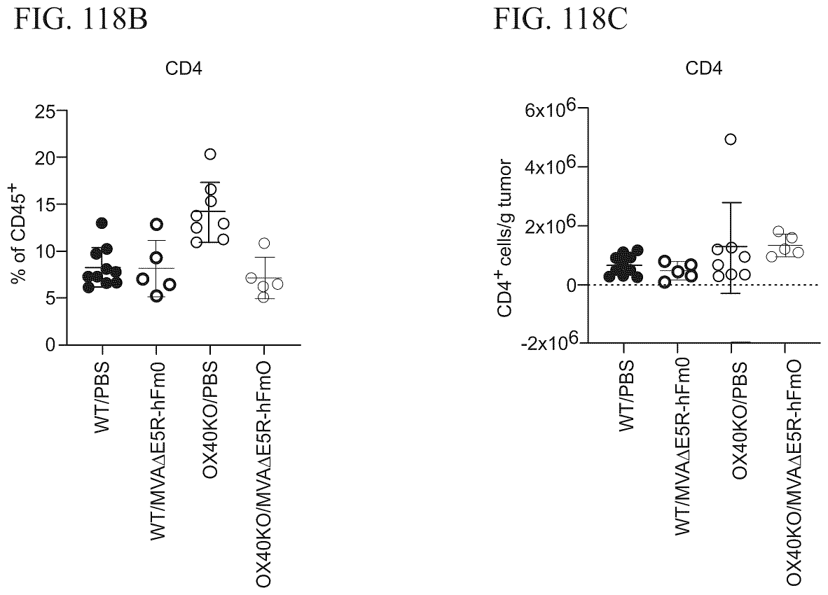

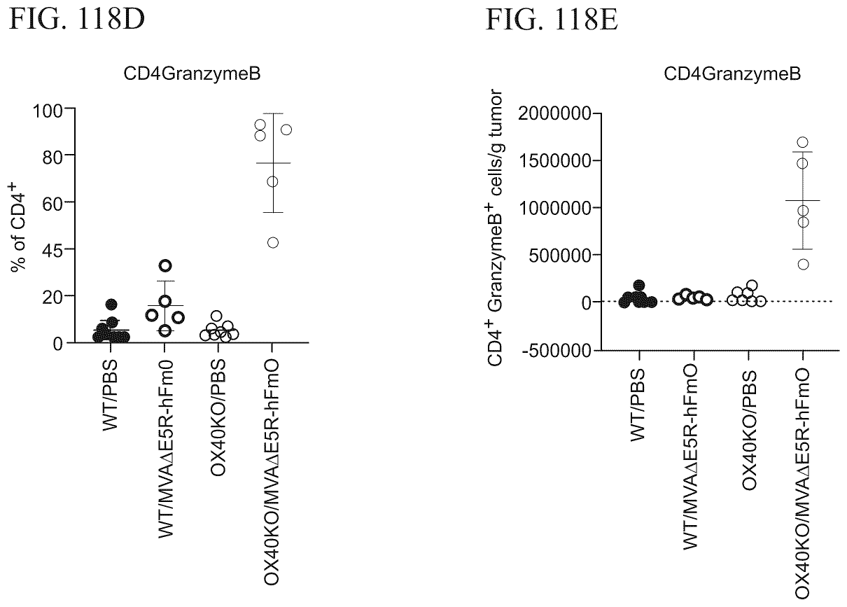

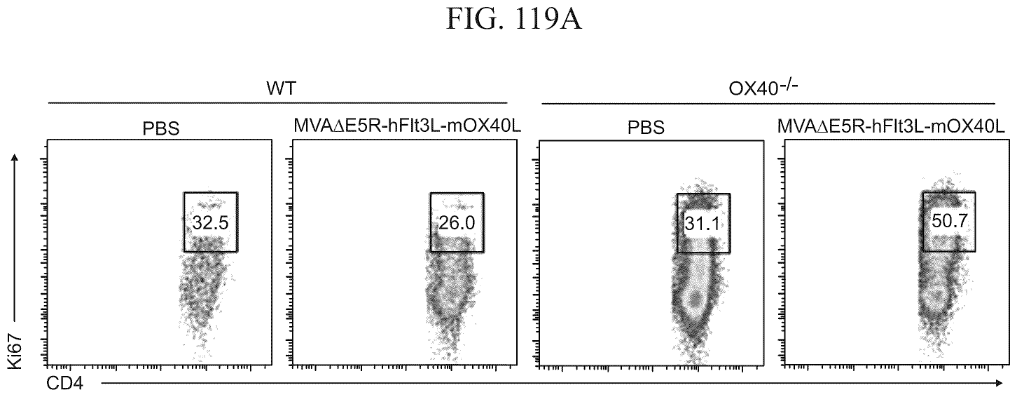

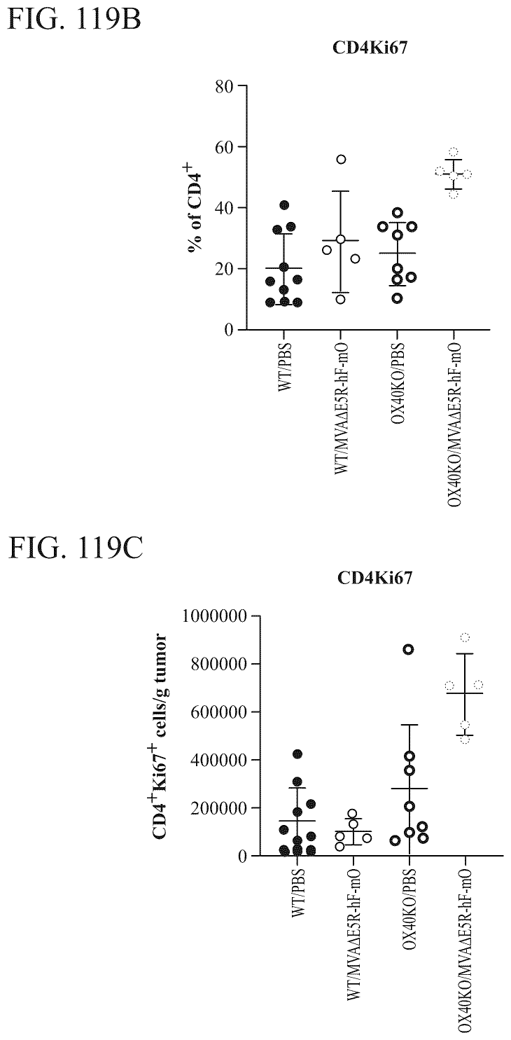

Similar observations were made when the percentages of Granzyme B⁺CD4⁺ and Ki67⁺CD4⁺ T cells out of CD4⁺ T cells in non-injected tumors from mice treated with IT MVAΔE5R-hFlt3L-mOX40L compared with those treated with PBS. IT MVAΔE5R-hFlt3L-mOX40L also elicited stronger activation and proliferation responses on CD4⁺ T cells in the non-injected tumors of OX40^{-/-} mice compared with those in WT mice (FIGS. 118A-119C).

These results suggest that OX40 negatively regulate immune responses induced by IT MVAΔE5R-hFlt3L-mOX40L at both injected and non-injected tumors. It is possible that OX40 is important for OX40⁺FoxP3⁺CD4⁺ T cells for their immune suppressive functions, which dampens both CD4⁺ and CD8⁺ effector T cell functions. These results suggest that combination of IT immunogenic and IFN-inducing MVA, such as MVAΔE5R, with systemic delivery of OX40 blocking agent, such as an anti-OX40L antibody, might elicit strong antitumor effects at both injected and non-injected tumors, and demonstrate that these viral constructs are useful in methods for treating solid tumors.

### Example 102: Intratumoral (IT) Injection of MVAΔE5R-hFlt3L-mOX40L Leads to Activation of Local CD8⁺ T Cells in the Injected Tumors in B16-F10 Bilateral Tumor Implantation Model

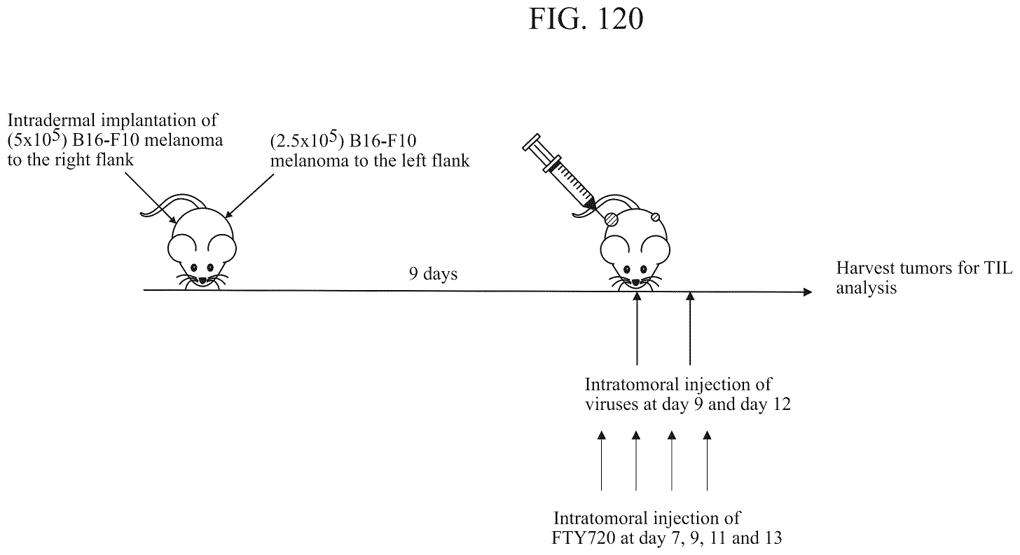

To assess whether blocking T cells trafficking from lymphoid organs to peripheral blood would affect antitumor effects elicited by IT MVAΔE5R-hFlt3L-mOX40L, a bilateral B16-F10 tumor implantation model was used and FTY720, an immunomodulatory drug that inhibits lymphocytes egress from lymphoid tissues (Figure. XX slide27). Briefly, B16-F10 melanoma cells were implanted intradermally into the shaved skin on the right (5×10⁵ cells) and left (2.5×10⁵ cells) flanks of C57BL/6J mice. Seven days post implantation, each mouse was injected intraperitoneally with 25 μg FTY720 or DMSO as control every other day. Nine days post implantation, the larger tumors on the right flank were injected twice per week with 4×10⁷ pfu of MVAΔE5R-hFlt3L-mOX40L or PBS, three days apart. Tumor growth was monitored. Two days post second injection, tumors were harvested and weighed. Cells were processed for surface labeling with anti-CD3, CD45, CD4, CD8 and OX40 antibodies, and also for intracellular Granzyme B and Ki67 staining. The live immune cell infiltrates in the tumors were analyzed by FACS (FIG. 120).

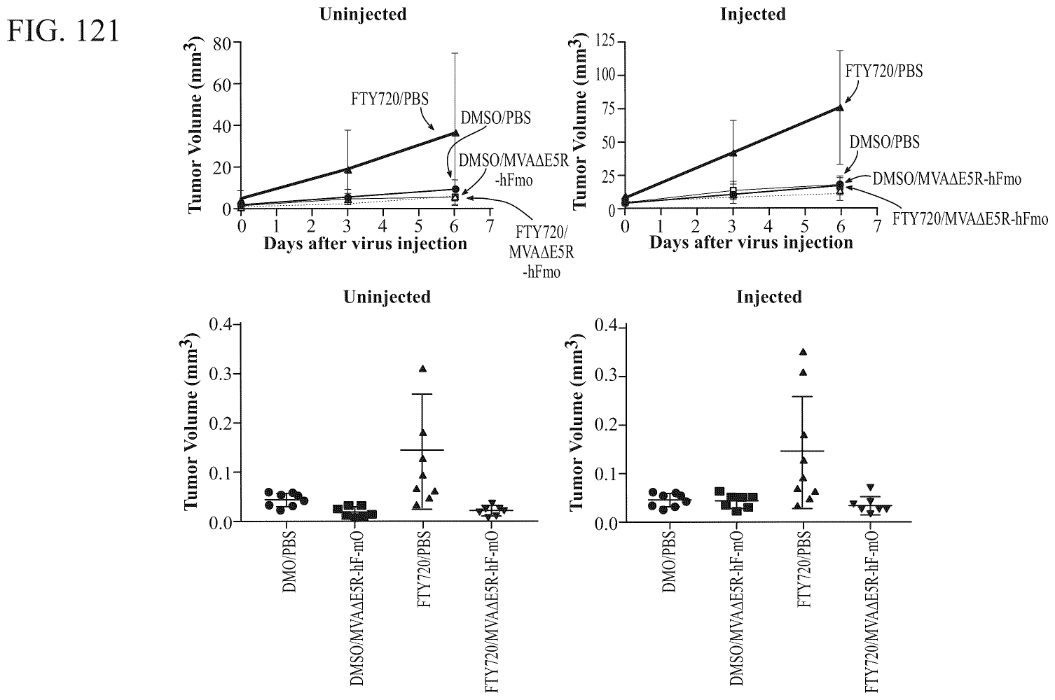

In PBS treated groups, both injected and non-injected tumors grew more aggressively in the presence of FTY720, compared with the DMSO control group (FIG. 121). By contrast, IT MVAΔE5R-hFlt3L-mOX40L inhibited the growth of both injected and non-injected tumors from both FTY720 or DMSO treated mice (FIG. 121).

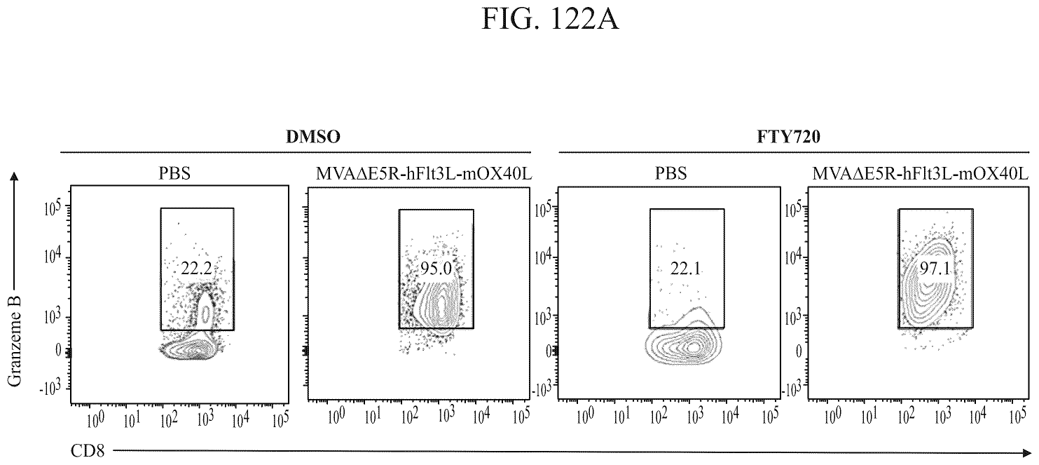

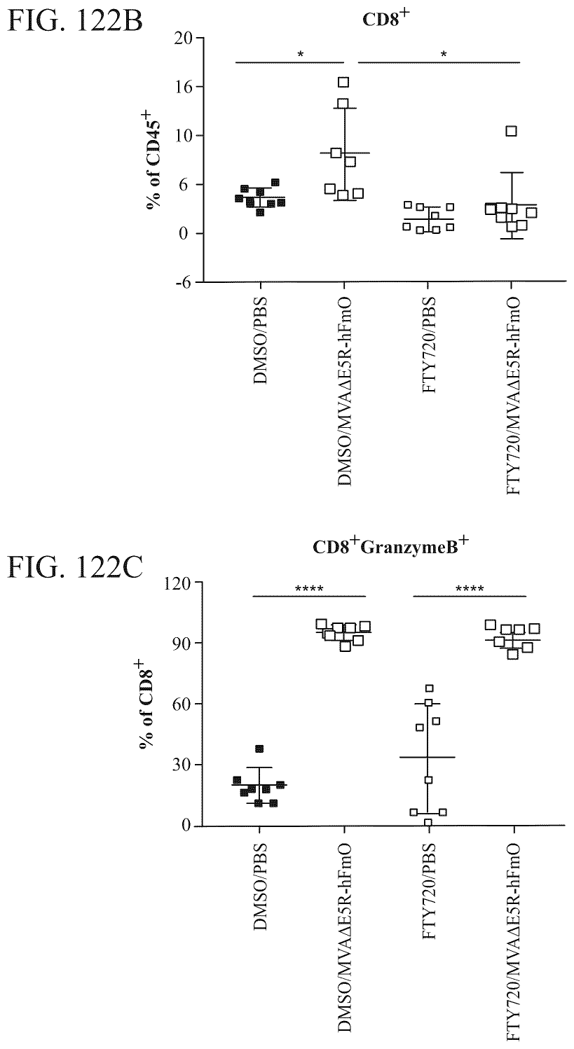

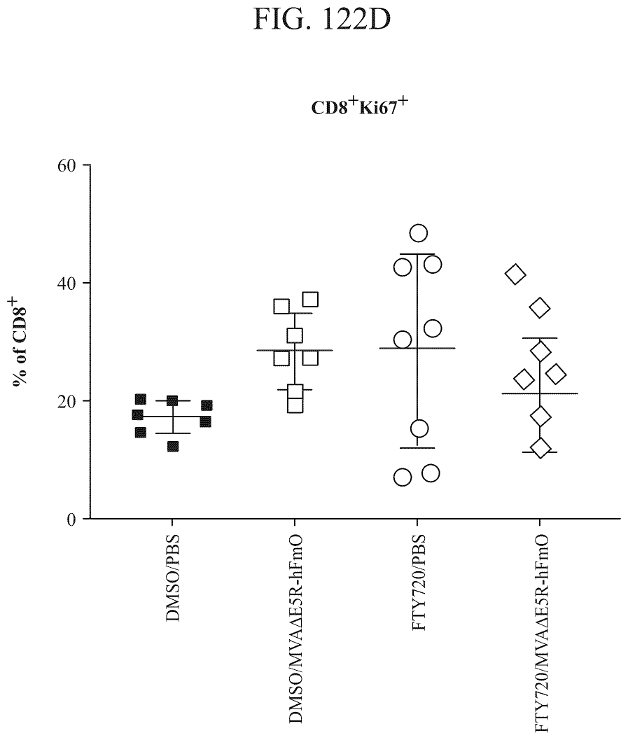

The percentages of CD8⁺ T cells out of CD45⁺ cells in the injected tumors were increased with IT MVAΔE5R-hFlt3L-mOX40L in DMSO control group. After FTY720 treatment, the percentages of CD8⁺ T cells out of CD45⁺ cells were slightly lower than DMSO/PBS group and IT MVAΔE5R-hFlt3L-mOX40L treatment did not increase the percentages of CD8⁺ T cells out of CD45⁺ cells (FIG. 122A-122D) XXB). By contrast, IT MVAΔE5R-hFlt3L-mOX40L resulted in significantly increased percentages of Granzyme B⁺ CD8⁺ T cells in both DMSO and FTY720 groups (FIGS. 122A and 122C). IT MVAΔE5R-hFlt3L-mOX40L also led to increased percentage of Ki67⁺CD8⁺ T cells in both DMSO and FTY720 groups (FIG. 122D).

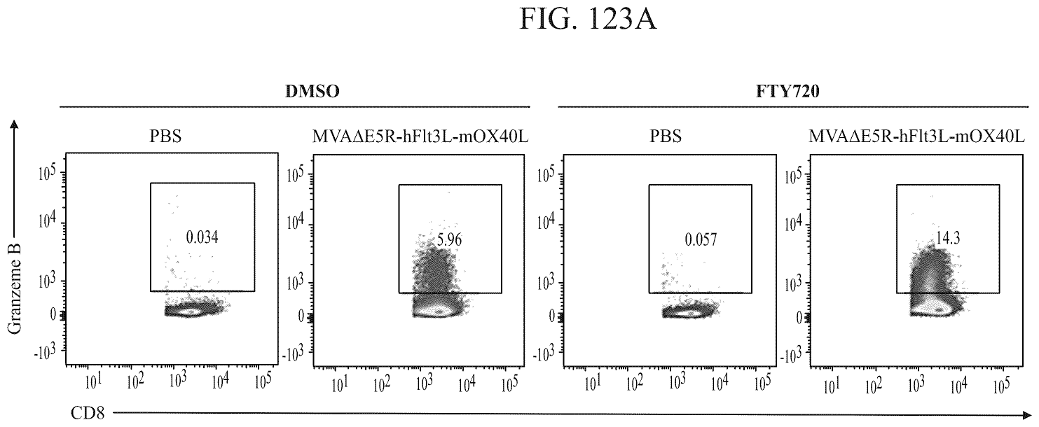

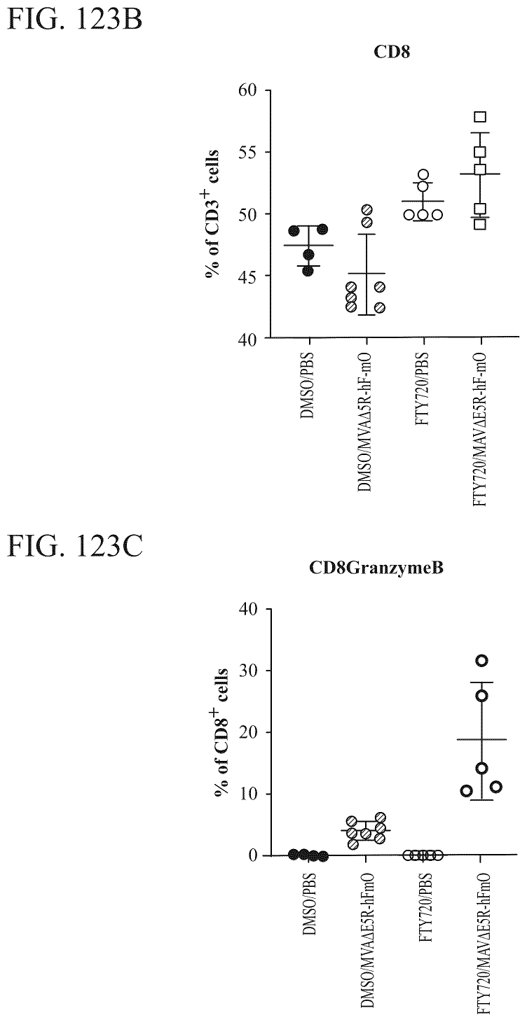

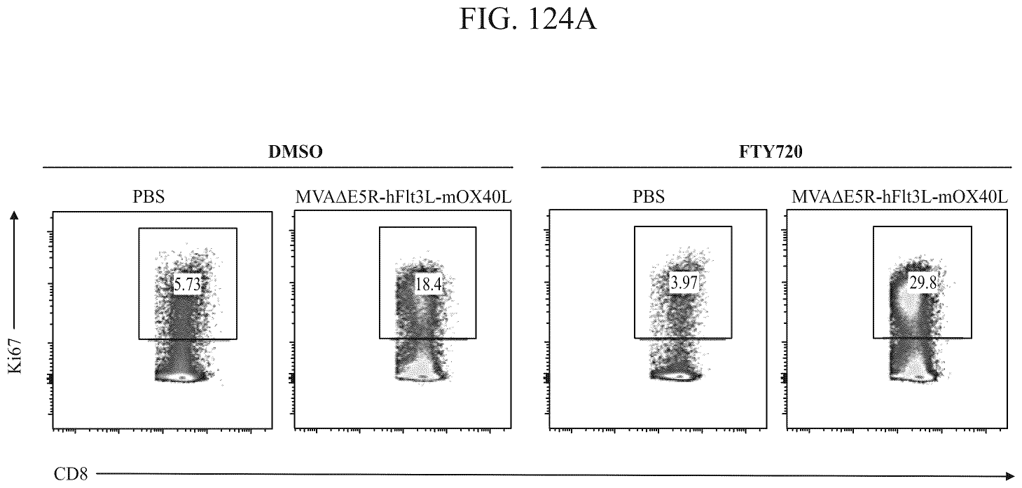

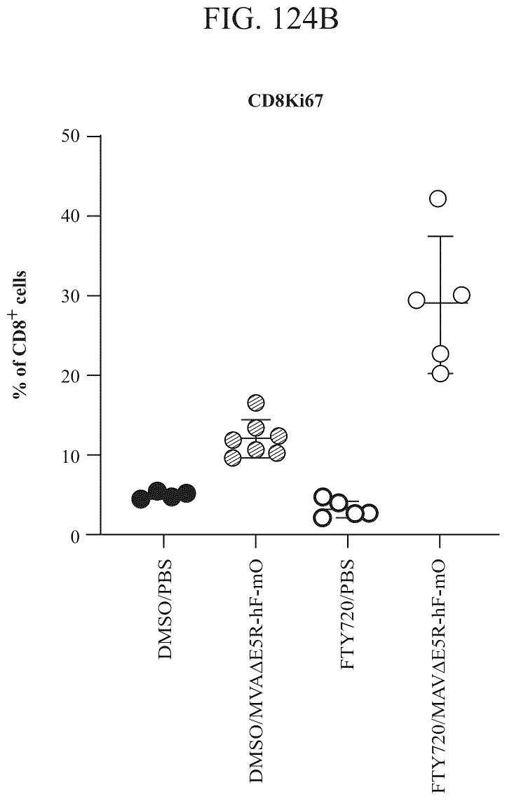

In addition, in the presence of FTY720, IT MVAΔE5R-hFlt3L-mOX40L resulted in higher percentages of activated Granzyme B⁺CD8⁺ and Ki67⁺CD8⁺ T cells in the TDLNs of injected tumors compared with DMSO (FIGS. 123A-124B), which indicated that the activated CD8⁺ T cells induced by IT MVAΔE5R-hFlt3L-mOX40L were trapped in LNs in the presence of FTY720. These results demonstrate that IT MVAΔE5R-hFlt3L-mOX40L activates local CD8⁺ T cells and is able to inhibit tumor growth even without recruiting T cells from lymphoid organs and is useful in methods for treating solid tumors. Accordingly, these results demonstrate that the recombinant poxviruses of the present technology are useful in methods for treating solid tumors.

### Example 103: Intratumoral (IT) Injection of MVAΔE5R-hFlt3L-mOX40L Delays Tumor Growth in AT3 Triple Negative Breast Cancer Model

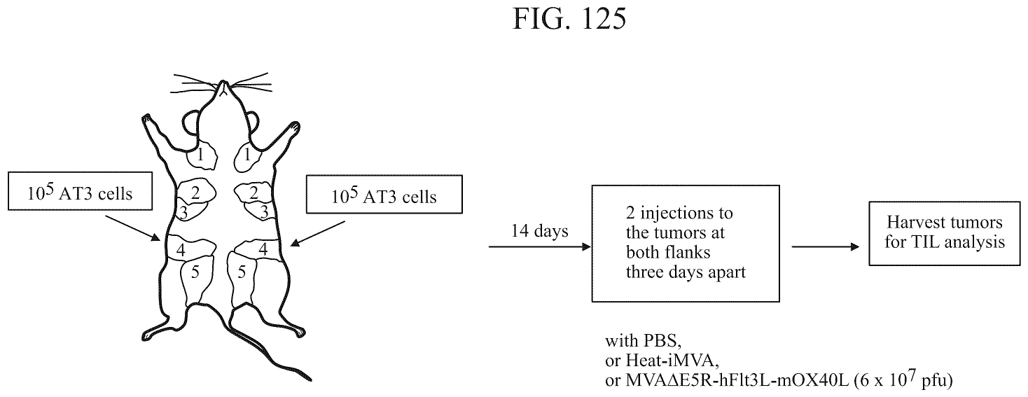

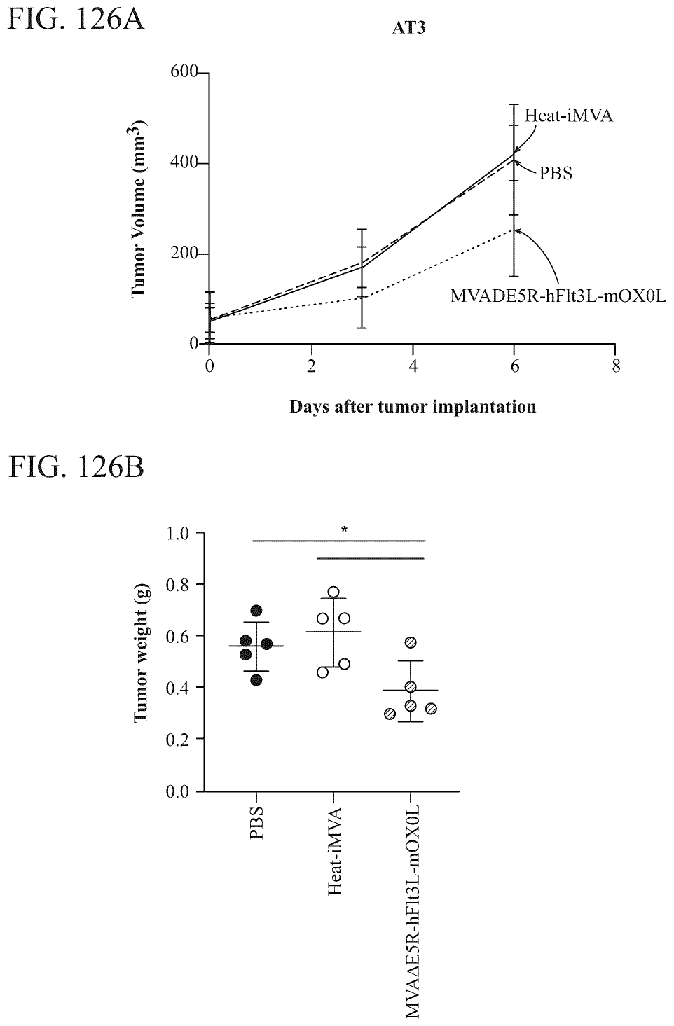

To assess the antitumor effects generated by IT injection of MVAΔE5R-hFlt3L-mOX40L in brast tumors, a bilateral AT3 murine breast tumor implantation model was used (FIG. 125). Briefly, 1×10⁵ AT3 cells were implanted to the 4 μl fat pad of a C57BL/6J mouse. 14 days post tumor implantation, 6×10⁷ pfu of MVAΔE5R-hFlt3L-mOX40L, or Heat-iMVA, or PBS were intratumorally injected twice, three days apart. The tumors were measured before each injection. Two days post second injection, the injected tumors were measured and harvested for weight measurement and FACS analysis. IT injection of MVAΔE5R-hFlt3L-mOX40L resulted in delayed tumor growth compared with Heat-iMVA or PBS (FIG. 126A). The tumors with IT MVAΔE5R-hFlt3L-mOX40L were smaller compared with Heat-iMVA or PBS after two injections (FIG. 126B). These results demonstrate that IT MVAΔE5R-hFlt3L-mOX40L resulted in stronger antitumor effect compared with Heat-iMVA in AT3 breast tumor implantation model. Accordingly, these results demonstrate that the recombinant poxviruses of the present technology are useful in treating solid tumors.

### Example 104: Intratumoral (IT) Injection of MVAΔE5R-hFlt3L-mOX40L Leads to Activation of CD8⁺ T Cells and Reduction of Regulatory T Cells in the Injected Tumors in AT3 Triple Negative Breast Cancer Model

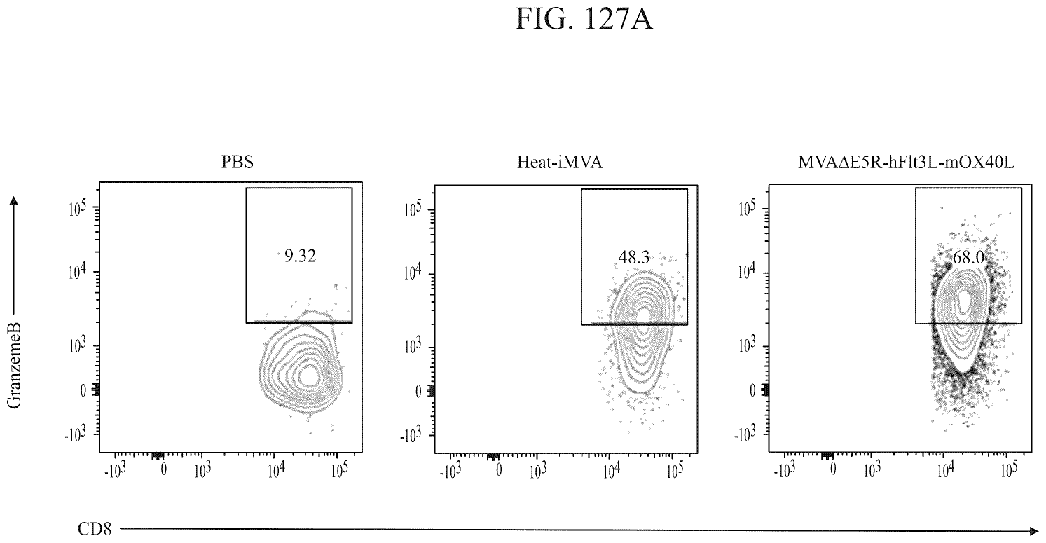

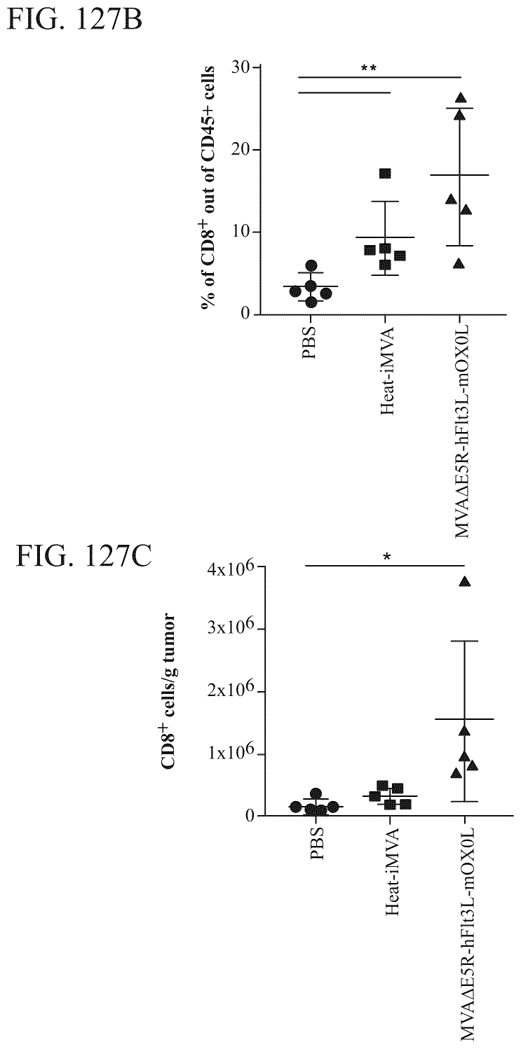

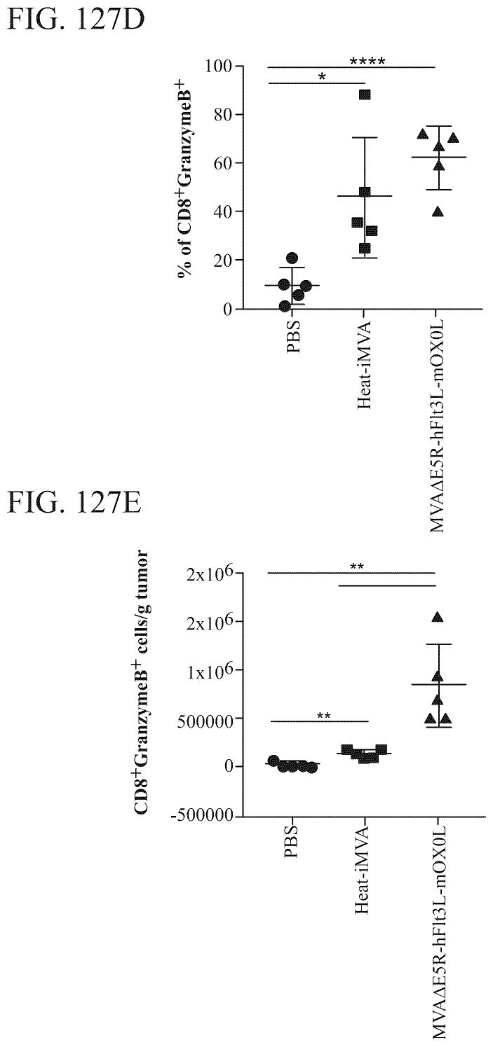

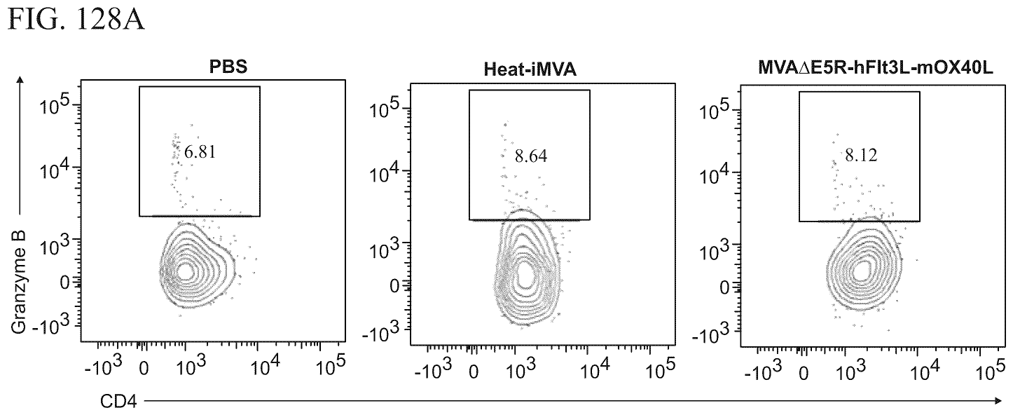

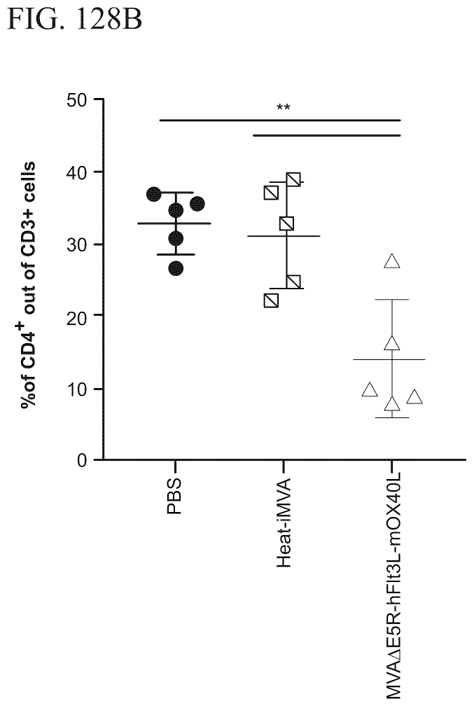

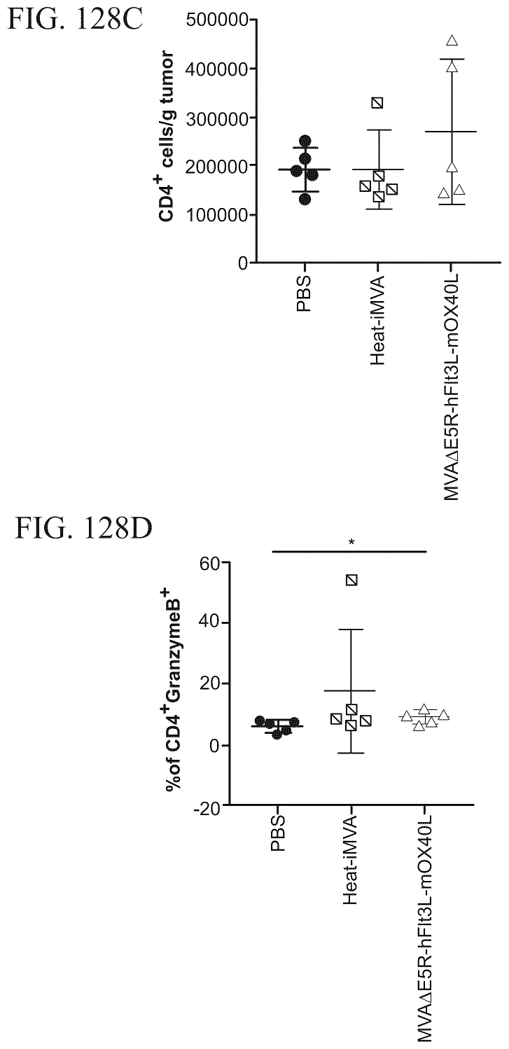

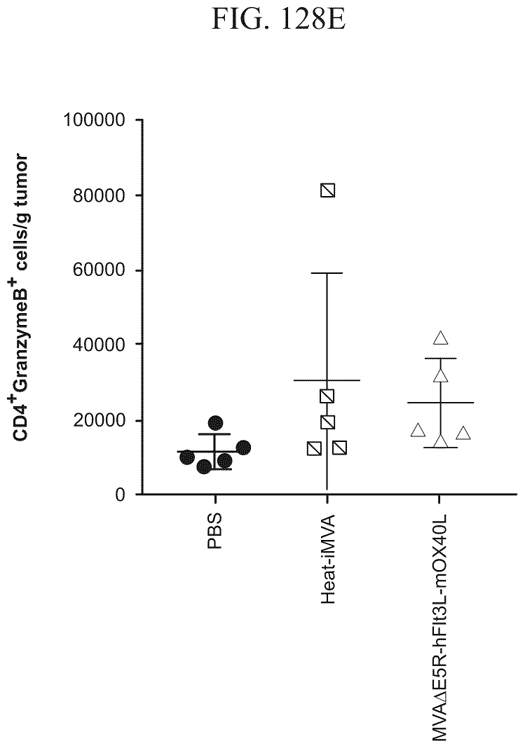

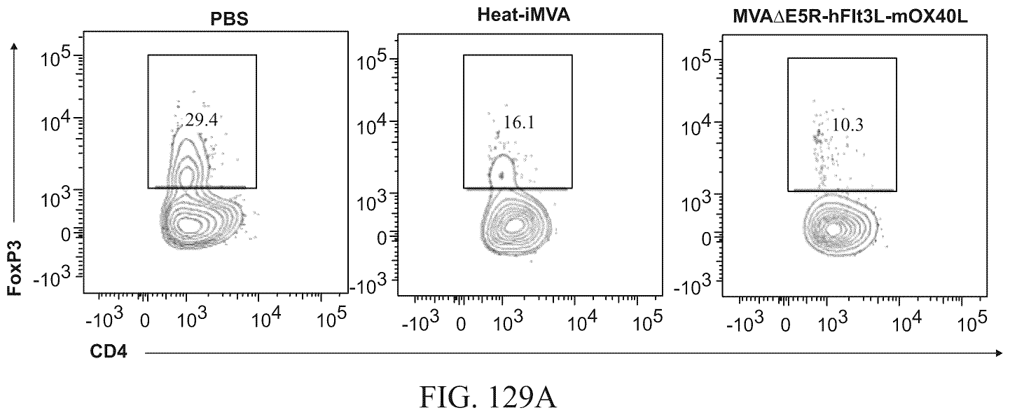

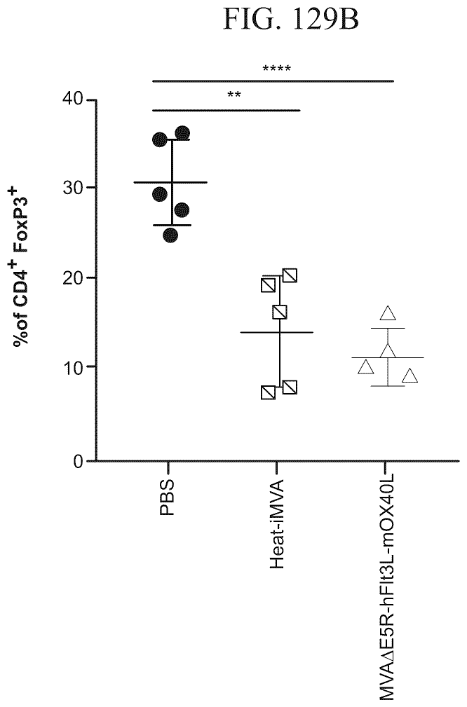

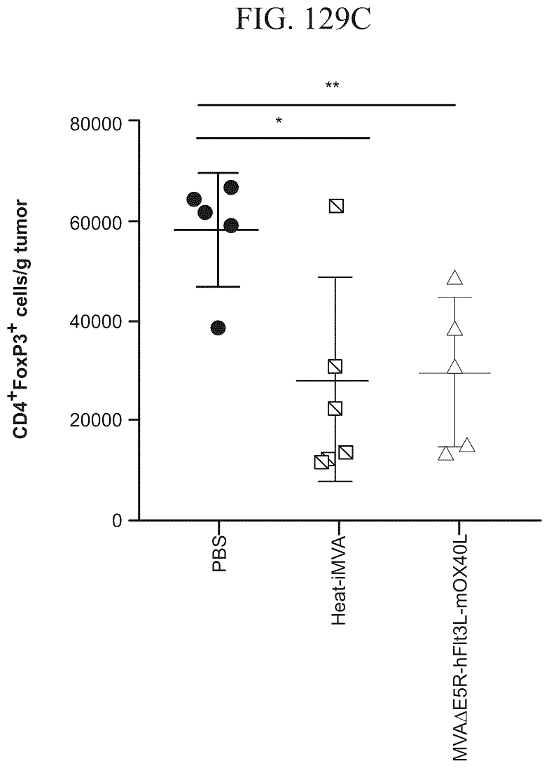

To assess the immune responses induced by IT injection of MVAΔE5R-hFlt3L-mOX40L in breast tumors, a bilateral AT3 murine breast tumor implantation model was used as described in Example 103 (FIG. 125). Two days post second injection, the injected tumors were harvested and cells were processed for surface labeling with anti-CD3, CD45, CD4 and CD8 antibodies, and also for intracellular Granzyme B and FoxP3 staining. IT injection of MVAΔE5R-hFlt3L-mOX40L resulted in higher percentages and absolute numbers of CD8⁺ T cells compared with Heat-iMVA or PBS (FIG. 127B). IT injection of MVAΔE5R-hFlt3L-mOX40L also induces higher percentages of GranzymeB⁺CD8⁺ T cells compared with Heat-iMVA (FIGS. 127A and 127C). However, neither IT injection of MVAΔE5R-hFlt3L-mOX40L or Heat-iMVA induced higher percentages of GranzymeB⁺CD4⁺ T cells (FIGS. 128A-128E). The percentage of CD4⁺FoxP3⁺ T cells was reduced from 31% to about 11% with IT MVAΔE5R-hFlt3L-mOX40L or Heat-iMVA (FIGS. 129A-129C). These results demonstrate that IT MVAΔE5R-hFlt3L-mOX40L leads to activation of CD8⁺ T cells and reduction of regulatory T cells in AT3 breast tumor implantation model, and is useful in methods for treating solid tumors.

### Example 105: The Combination of Intratumoral Injection of MVAΔE5R-hFlt3L-mOX40L and Systemic Delivery of Anti-PD-L1 Antibody Cures Bilateral B16-F10 Melanoma

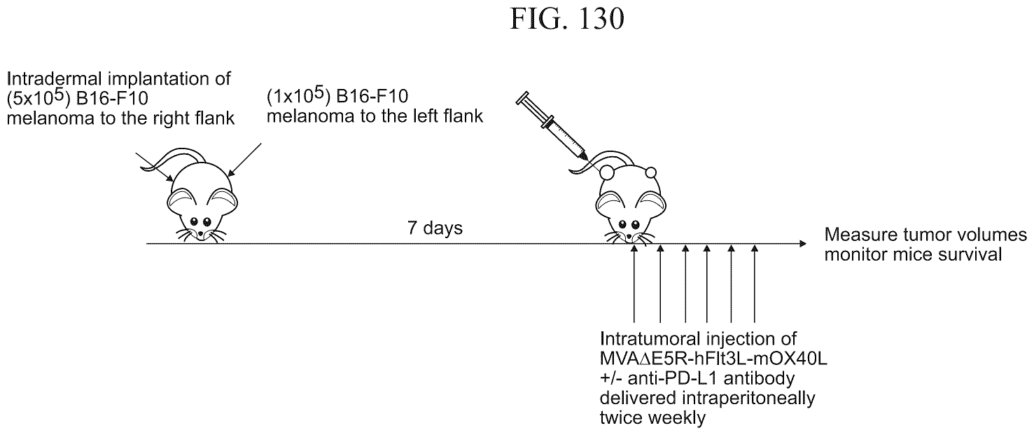

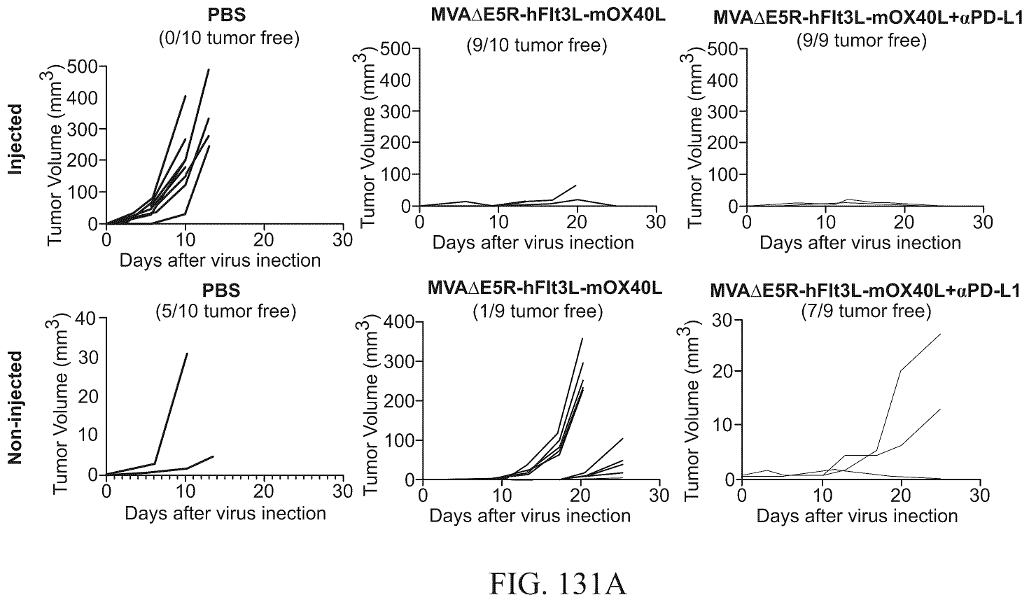

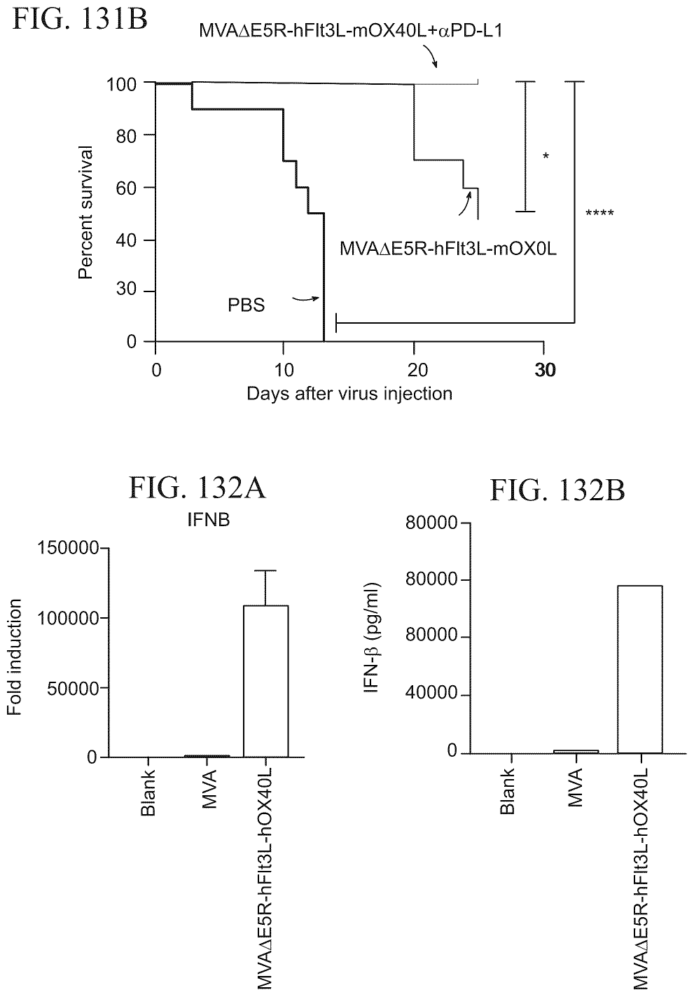

To test whether the combination with IT delivery of MVAΔE5R-hFlt3L-mOX40L and systemic delivery of anti-PD-L1 had superior anti-tumor efficacy compared with IT virus alone, a bilateral murine B16-F10 tumor implantation model was used. Briefly, B16-F10 melanoma cells were implanted intradermally to the left and right flanks of C57B/6 mice (5×10⁵ to the right flank and 1×10⁵ to the left flank). Seven days after tumor implantation, MVAΔE5R-hFlt3L-mOX40L (4×10⁷ PFU) was delivered into the larger tumors on the right flank twice weekly, with concomitant intraperitoneal (IP) injection of with anti-PD-L1 (250 μg per mouse). Tumor sizes were measured twice a week and mice survival were monitored (FIG. 130). The volumes of injected and non-injected tumors of individual mouse are shown in FIG. 131A-131B). In mice treated with PBS, tumors grew rapidly, which resulted in early death with a median survival of 12.5 days (FIGS. 131A-132B). Intratumoral injection of MVAΔE5R-hFlt3L-mOX40L resulted in delayed tumor growth and improved survival compared with PBS, with an extension of median survival to 25 days (FIGS. 131A-131B). Mice treated with the combination of intratumoral delivery of MVAΔE5R-hFlt3L-mOX40L and intraperitoneal delivery of anti-PD-L1 were all surviving at the time of last survey. Seven out of nine mice were tumor free and were expected to be cured of B16-f10 melanoma (FIGS. 131A-131B). Based on previous combination therapy of IT MVAΔC7L-hFlt3L-TK(−)-mOX40L and systemic delivery of anti-CTLA-4 or anti-PD-L1 in both bilateral tumor model or a large established tumor model, without being bound by theory, it is expected that the combination of IT delivery of MVAΔE5R-hFlt3L-mOX40L and systemic delivery of anti-anti-CTLA-4 or anti-PD-1 should generate superior results compared with IT virus alone in both bilateral tumor model or a large established tumor model.

### Example 106: MVAΔE5R-hFlt3L-OX40L Induces Higher Levels of IFNB Gene Expression Compared with MVA

The induction of IFNB gene expression and IFN-β secretion by MVAΔE5RhFlt3L-hOX40L vs. MVA-infected BMDCs was examined. Briefly, BMDCs (1×10⁶) were infected with either MVAΔE5RhFlt3L-hOX40L or MVA at a MOI of 10. Cells were washed after 1 h infection and fresh medium was added. Cells were collected at 6 h post infection and supernatants were collected at 19 h post infection. IFNB gene expressions was determined by RT-PCR (FIG. 132A). IFN-β protein levels in the supernatants were determined by ELISA (FIG. 132B). RT-PCR results show that MVAΔE5RhFlt3L-hOX40L strongly induces IFNB gene expression and IFN-β secretion (FIGS. 132A and B).

### Example 107: Ex Vivo Infection with MVAΔE5R-hFlt3L-hOX40L

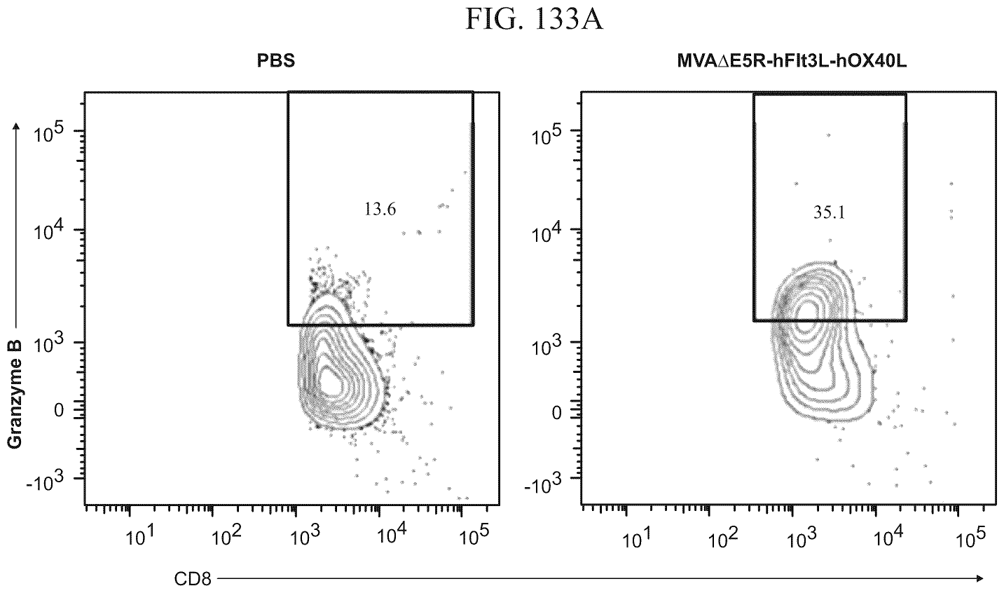

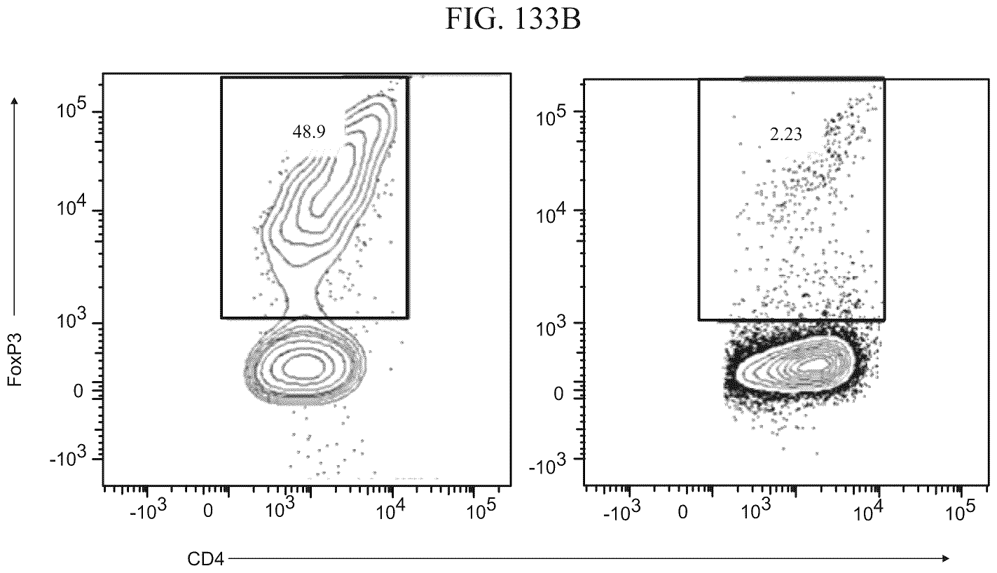

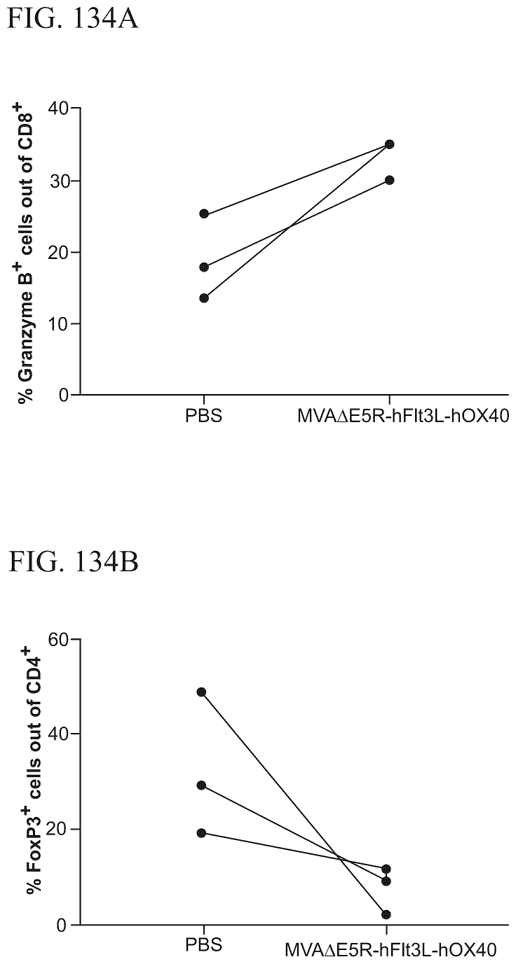

Extramammary Paget's disease (EMPD) is a rare, slow growing, skin cancer, which occurs in the epithelium and often originates from apocrine glandular cells at the vulva, scrotum, or perianal area. It is usually limited to the epithelium, but it can progress and become invasive. The treatment option is often limited, which includes surgery, radiotherapy, topical imiquimod, photodynamic therapy. How ex vivo culture of biopsy specimen with MVAΔE5R-hFlt3L-hOX40L affects the phenotype of tumor-infiltrating lymphocytes in EMPD was analyzed. Briefly, tumor tissues were cut into small pieces with sharp razors and infected with MVAΔE5R-hFlt3L-hOX40L at a MOI of 10. After two days of infection, tissues were digested with collagenase D ( ) at 37° C. for 45 min. Cells were filtered and stained with anti-CD3, CD4, CD8 antibodies and were subsequently permeabilized and stained with anti-Granzyme B, and FoxP3 antibodies. FACS analysis was performed. Representative dot plots of Granzyme B⁺CD8⁺ T cells and FoxP3⁺CD4⁺ T cells are shown (FIGS. 133A and 133B). FIG. 134A shows a graph of percentages of Granzyme*CD8⁺ T cells out of CD8⁺ cells after infection with MVAΔE5R-hFlt3L-hOX40L or PBS control for two days. Data are means±SEM (n=3). FIG. 134B shows a graph of percentages of FoxP3⁺CD4⁺ T cells T cells out of CD4⁺ cells after infection with MVAΔE5R-hFlt3L-hOX40L or PBS control for two days. Data are means±SEM (n=3). These results show that ex vivo infection of EMPD with MVAΔE5R-hFlt3L-hOX40L results in the increase of the percentages of Granzyme⁺CD8⁺ T cells out of CD8⁺ cells and the reduction of the percentages of FoxP3⁺CD4⁺ T cells out of CD4⁺ cells (FIGS. 134A and 134B), supporting immune-stimulating function of MVAΔE5R-hFlt3L-hOX40L. Accordingly, these results demonstrate that the recombinant poxviruses of the present technology are useful in methods for treating solid tumors.

### Example 108: Generation of MVAΔE3LΔE5R and MVAΔE3LΔE5R hFlt3L-mOX40L Recombinant Viruses

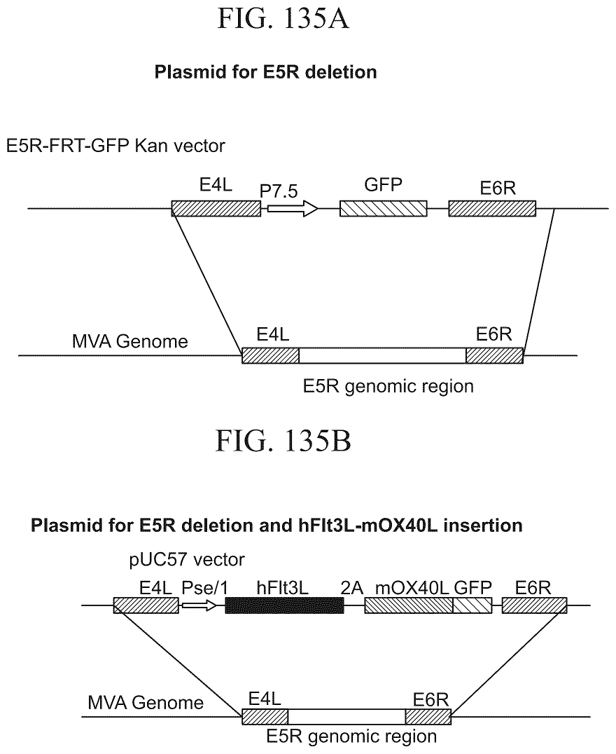

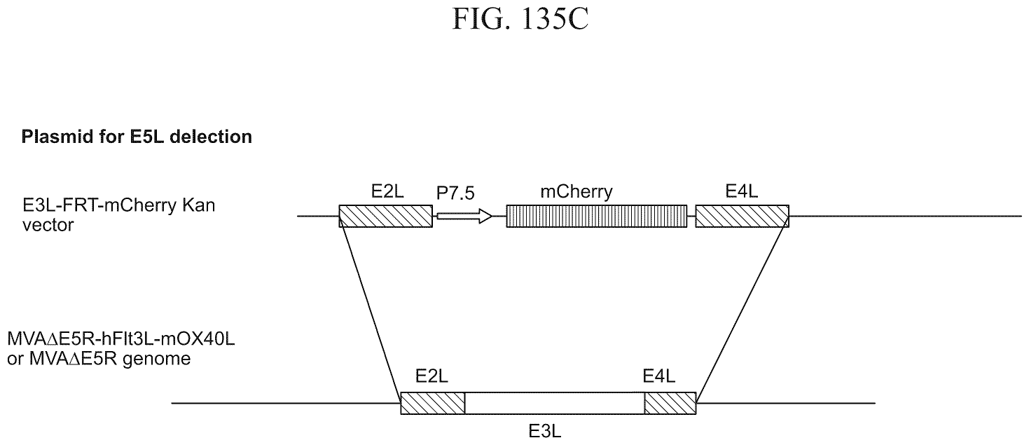

In order to test whether deletion of vaccinia E3L gene from MVAΔE5R or MVAΔE5RhFlt3L-mOX40L improves the immunogenicity of the viruses, MVAΔE3LΔE5R and MVAΔE3LΔE5R hFlt3L-mOX40L were generated. The process consisted of multiple steps. In the first step, MVAΔE5R and MVAΔE5R hFlt3L-mOX40L were generated through homologous recombination at the E4 and E6 loci of the MVA genome (FIGS. 135A and 135B). In the second step, an E3L-mCherry FRT construct was used to remove endogenous E3L from the recombinant viruses through homologous recombination at the E2L and E4L loci (FIG. 135C). The proper integration and purity of the mCherry construct inserted into the E3L locus was confirmed by PCR. The resulting virus lacked both E3L and E5R genes. In order to facilitate later steps in engineering, FRT sites were included in the construct used for E3L deletion. That construct had one FRT site proximal to the p7.5 promoter and another FRT site distal to mCherry. In the third step, this virus was allowed to replicate in cells expressing Flp recombinase in order to remove the mCherry from the viral genome, when further engineering of the virus was desired.

### Example 109: MVAΔE3LΔE5R Induces Higher Levels of IFNB Gene Expression in BMDCs Compared with MVAΔE5R and the Induction is Largely Dependent on cGAS

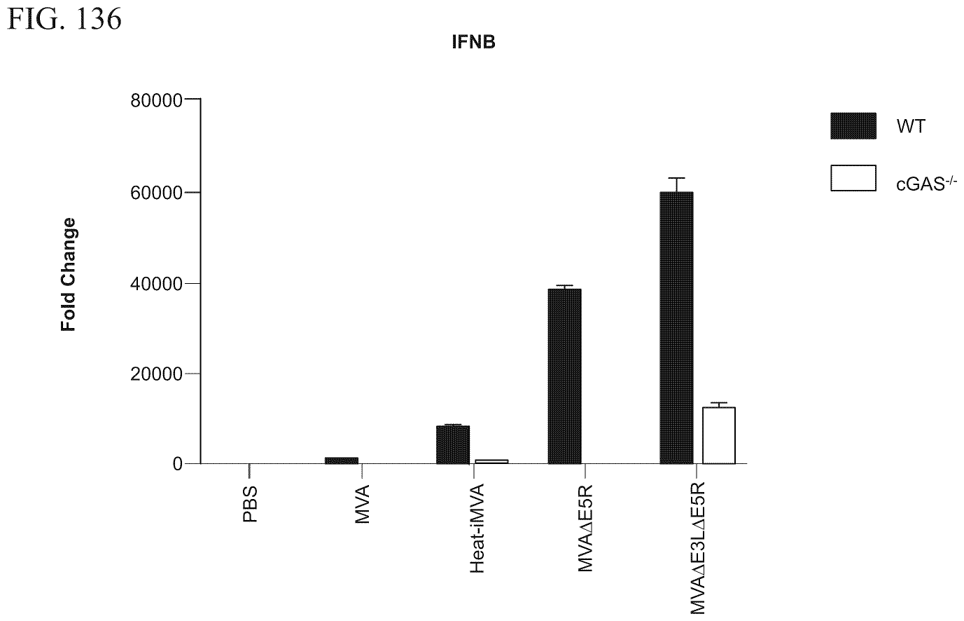

Vaccinia E3 is an important virulence factor with a N-terminal Z-DNA and C-terminal dsRNA-binding domains. Intranasal infection of VACVΔE3L is non-pathogenic in an intranasal infection model. MVAΔE3L induces higher levels of type I IFN compared with MVA (Dai et al., Plos Pathogens 2014). To test whether MVAΔE3LΔE5R induces higher levels of type I IFN compared with MVAΔE5R, Heat-iMVA, or MVA, BMDCs (1×10⁶) from WT C57BL/6J and cGAS^{-/-} mice were infected with either MVAΔE3LΔE5R, MVAΔE5R, Heat-iMVA, or MVA at a MOI of 10. Cells were collected at 6 h post infection. IFNB gene expression was determined by RT-PCR. The results show that MVAΔE3LΔE5R induces higher levels of IFNB gene expression compared with MVAΔE5R in WT BMDCs. Whereas MVAΔE5R-induced IFNB gene expression is completely lost in cGAS^{-/-} cells, MVAΔE3LΔE5R-induced IFNB gene expression is largely reduced in cGAS^{-/-} cells, suggesting that additional pathway such as the MDA5/MAVS-mediated cytosolic dsRNA-sensing pathway might play a minor role in detecting dsRNA produced by this virus in BMDCs (FIG. 136).

### Example 110: MVAΔE3LΔE5R Infection of Murine B16-F10 Melanoma Cells Strongly Induces IFNB Gene Expression and IFN-β Protein Secretion

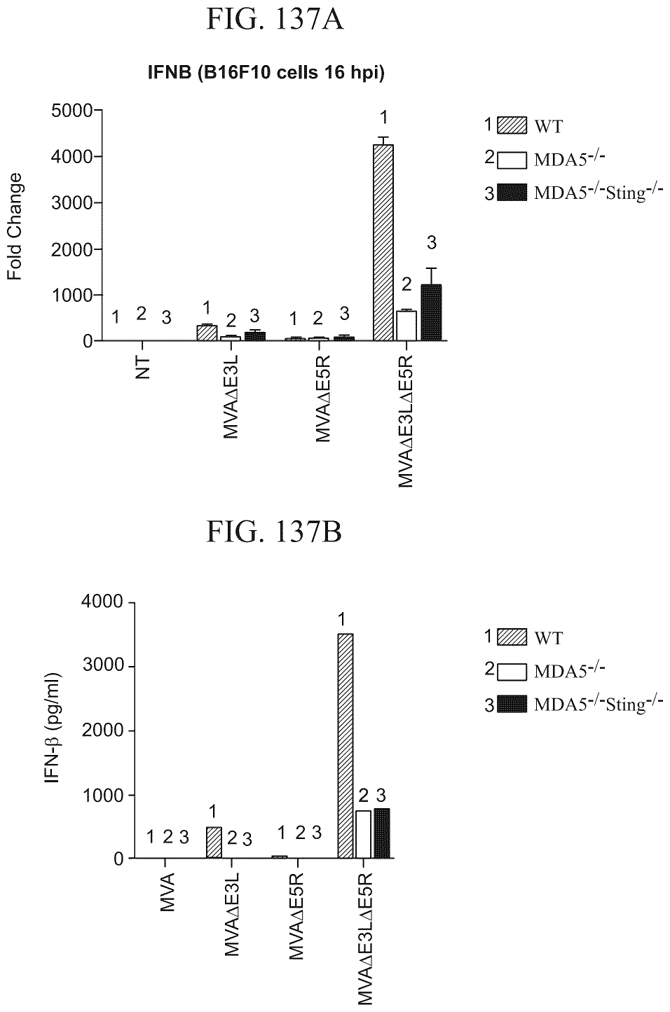

Whether MVAΔE3LΔE5R and MVAΔE5 could induce IFNB gene expression and protein secretion in murine B16-F10 melanoma cells was tested. B16-F10 cells were infected with either MVAΔE3L, MVAΔE5R or MVAΔE3LΔE5R at a MOI of 10. Cells were collected at 15 h post infection. Supernatants were collected at 24 h post infection. RT-PCR analysis showed that MVAΔE3LΔE5R infection of B16-F10 murine melanoma cells induces very strong induction of IFNB (4000 fold) compared to MVAΔE3L or MVAΔE5R (300 or 50-fold respectively). This difference was highly significant (p<0.0001) (FIG. 137A). This indicates that the deletions of E3L and E5R genes have a synergistic effect. ELISA results showed that MVAΔE3LΔE5R infection of B16-F10 cells induces much higher levels of IFN-β protein levels in the supernatants of infected cells compared with those infected with MVAΔE3L (3498 μg/ml vs. 479 μg/ml, respectively). MVAΔE5R fails to induce IFN-β protein secretion in B16-F10 cells. To test which nucleic acid-sensing pathways are important for detecting MVAΔE3LΔE5R infection in B16-F10 cells, MDA5^{-/-} and MDA5^{-/-} Sting^{-/-} B16-F10 cell lines were generated using CRISPR-cas9 and validated the loss of the respective proteins and genes using both Western Blot against targeted proteins and sequencing of targeted exons. These results show that the strong induction of IFNB by MVAΔE3LΔE5R is largely MDA5-dependent as shown by markedly reduced levels in MDA5^{-/-} cells in RT-PCR and ELISA.

### Example 111: MVAΔE3LΔE5R-hFlt3L-mOX40L Infection of Murine B16-F10 Melanoma Cells Strongly Induces hFlt3L and mOX40L Expression on the Surface of Infected Cells

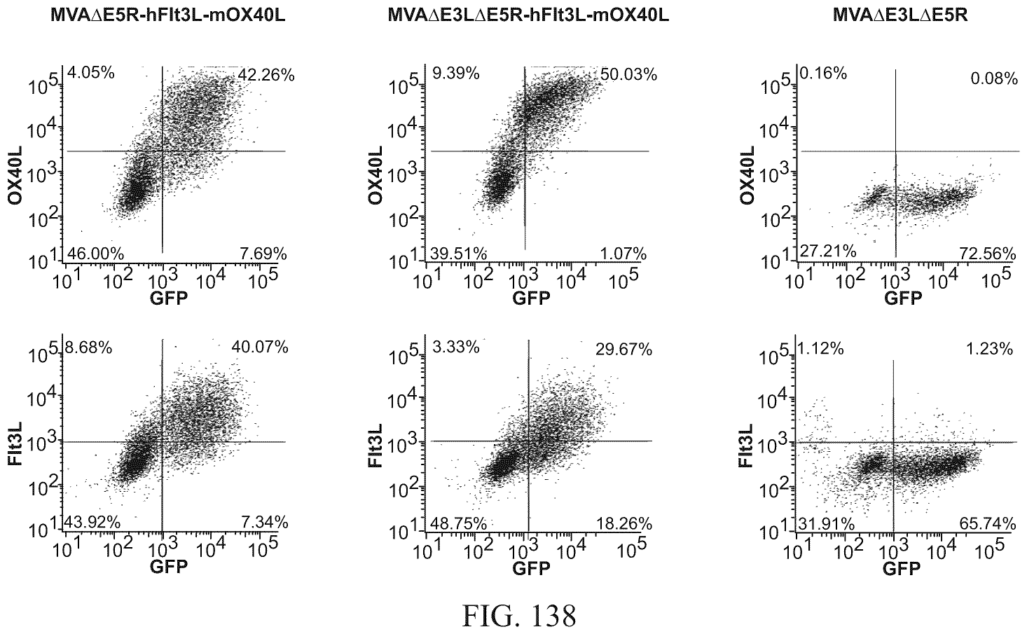

To test whether deletion of the E3L gene affected the expression of hFlt3L or mOX40L, B16-F10 cells were infected with either MVAΔE3LΔE5R-hFlt3L-mOX40L, MVAΔE5R-hFlt3L-mOX40L, or MVAΔE3LΔE5R for 1h. Cells were washed and incubated in fresh medium and harvested 24 hour later. Cells were stained with anti-hFlt3L and anti-mOX40L antibodies and FACS was performed. FIG. 138 shows representative dot plots from FACS demonstrating the expression of mOX40L or hFlt3L cells on the surface of B16-F10 cells infected with either MVAΔE3LΔE5R hFlt3L-mOX40L or MVAΔE5R hFlt3L-mOX40L. The control virus MVAΔE3LΔE5R-infected B16-F10 cells fail to express hFlt3L and mOX40L as expected. These results indicate that deletion of the E3L gene fails to affect the expression of the two transgenes hFlt3L and mOX40L.

### Example 112: Intratumoral Delivery of MVAΔE3LΔE5R-hFlt3L-mOX40L Induces Stronger Antitumor Systemic T Cell Responses Compared with MVAΔE5R-hFlt3L-mOX40L

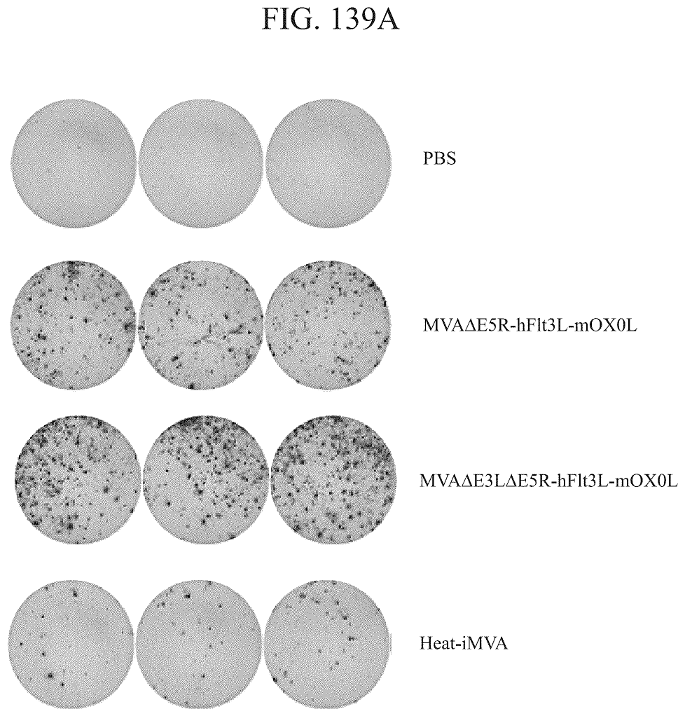

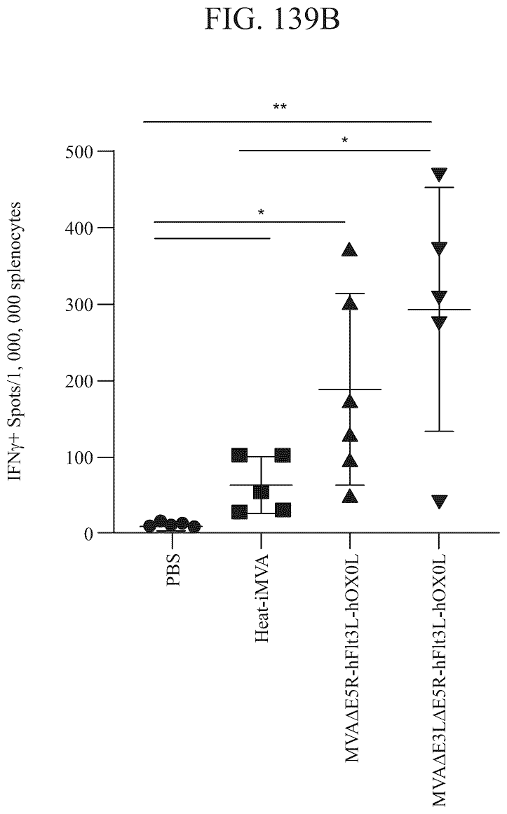

Given that infection of BMDCs and B16-F10 with MVAΔE3LΔE5R induces stronger type I IFN production compared with MVAΔE5R through activating both the cytosolic DNA-sensing pathway mediated by cGAS/STING and the cytosolic dsRNA-sensing pathway mediated by MDA5/MAVS, without being bound by theory, it is hypothesizes that IT delivery of MVAΔE3LΔE5R-hFlt3L-mOX40L virus would induce stronger antitumor immune responses. To test that, B16-F10 melanoma cells were implanted intradermally to the left and right flanks of C57B/6J mice (5×10⁵ to the right flank and 2.5×10⁵ to the left flank). Seven days post tumor implantation, 2×10⁷ pfu of either MVAΔE5R-hFlt3L-mOX40L, MVAΔE3LΔE5R-hFlt3L-mOX40L, an equivalent amount of Heat-iMVA, or PBS was intratumorally (IT) injected into the larger tumors on the right flank twice, three days apart. Spleens were harvested at 2 days post second injection, ELISPOT analyses were performed to evaluate tumor-specific T cells in the spleens. ELISPOT assay was performed by co-culturing irradiated B16-F10 cells (150,000) and splenocytes (1,000,000) in a 96-well plate. FIG. 139A shows the image of ELISPOT of triplicate samples of combined splenocytes from mice in the same treatment group. FIG. 139B shows a graph of IFN-γf spots per 1,000,000 splenocytes. These results show that IT MVAΔE3LΔE5R-hFlt3L-mOX40L generated the strongest antitumor T cell responses in the spleens of treated mice compared with either MVAΔE5R-hFlt3L-mOX40L, or Heat-iMVA. These results suggest that the activation of cytosolic dsRNA-sensing pathway mediated by MDA5/MAVS in the tumors might be important for generating strong antitumor adaptive immune responses.

### Example 113: Intratumoral Delivery of MVAΔE3LΔE5R-hFlt3L-mOX40L Delays B2M-Deficient B16-F10 Cells

Mutations in Beta 2 microglobulin (B2M) gene have been observed in tumors that relapse with resistance after immune checkpoint blockade therapy. To test whether MVAΔE3LΔE5R-hFlt3L-mOX40L is efficacious against such tumors, a B2M deficient B16F10 tumor model was generated using CRISPR-cas9 technology. Beta 2 Microglobulin (B2M) is an essential component of the MHC Class I complex. FACS analysis confirmed that only cells transfected with anti-B2M gRNAs lost surface MHC at high frequency, indicating an effective CRISPR (data not shown). Cell sorting was used to isolate cells lacking surface MHC class I. A single clonal isolate from this sorting was selected for sequencing and subsequent in vivo experiments. This B2M^{-/-} clonal isolate had a 178 BP deletion in exon 2 of B2M which eliminates half the coding sequence of B2M and creates a frame shift (data not shown).

WT and B2M^{-/-} B16-F10 melanoma cells (2.5×10⁵) were implanted intradermally to the right flanks of C57B/6J mice. In order for B2M tumors to implant successfully, NK cells were depleted using PK136 antibody (200 μg/mouse on days −1, 2 and 5) in mice implanted with either WT or B2M^{-/-} B16-F10 cells. Tumors were allowed to grow for 10 days. Afterwards, 4×10⁷ pfu of MVAΔE3LΔE5R hFlt3L-mOX40L or PBS was intratumorally (IT) injected twice weekly. The tumor sizes were measured and the survival of mice was monitored (FIG. 140).

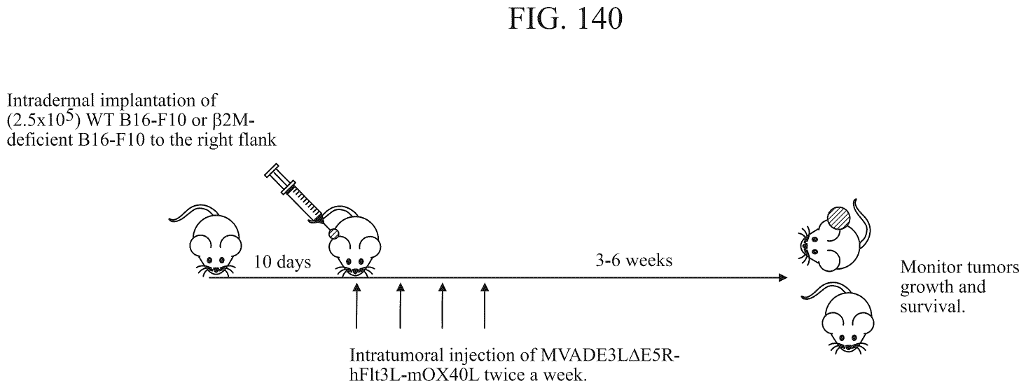

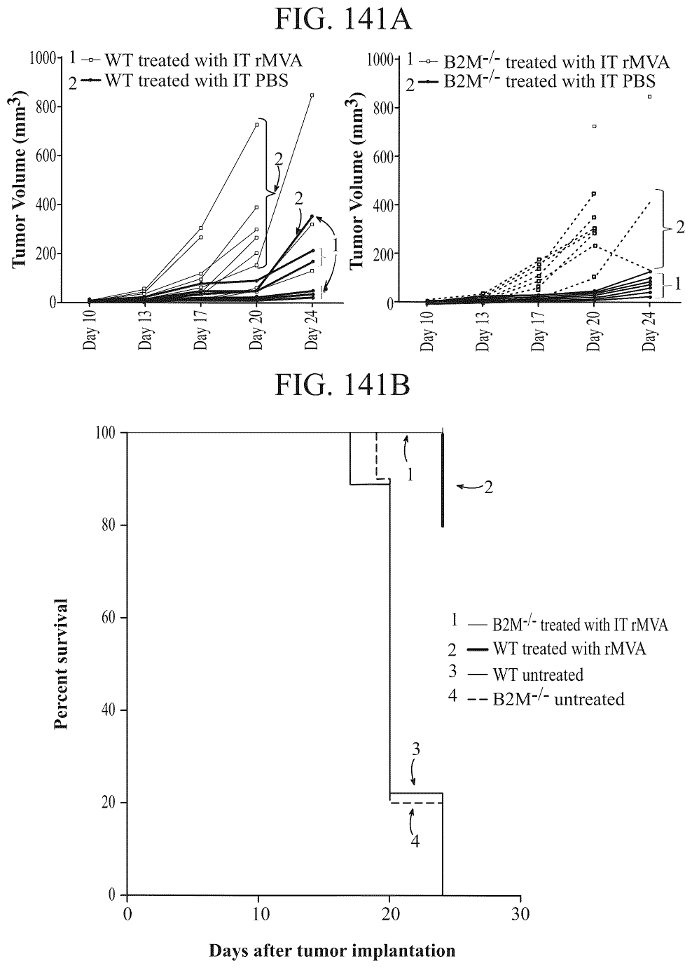

FIG. 141A shows tumor volumes of the injected WT and B2M^{-/-} B16-F10 tumors in mice treated with either PBS or MVAΔE3LΔE5R-hFlt3L-mOX40L intratumorally. Both WT and B2M^{-/-} B16-F10 tumors responded to MVAΔE5R-hFlt3L-mOX40L treatment with delaying of tumor growth. FIG. 141B shows the Kaplan Meier survival curve of the four groups. IT MVAΔE3LΔE5R hFlt3L-mOX40L virus generated an unequivocal survival benefit to mice bearing B2M deficient or WT B16-F10 tumors. By day 24, none of the untreated mice survived while all of the mice bearing B2M^{-/-} tumors treated with MVAΔE3LΔE5R-hFlt3L-mOX40L were alive. Accordingly, these results demonstrate that the recombinant poxviruses of the present technology are useful in methods for treating solid tumors.

### Example 114: Intratumoral (IT) Injection of MVAΔE3LΔE5R-hFlt3L-mOX40L Induces CD8⁺ T Cell Activation and Proliferation in the Injected Tumors in AT3 Triple Negative Breast Cancer Model

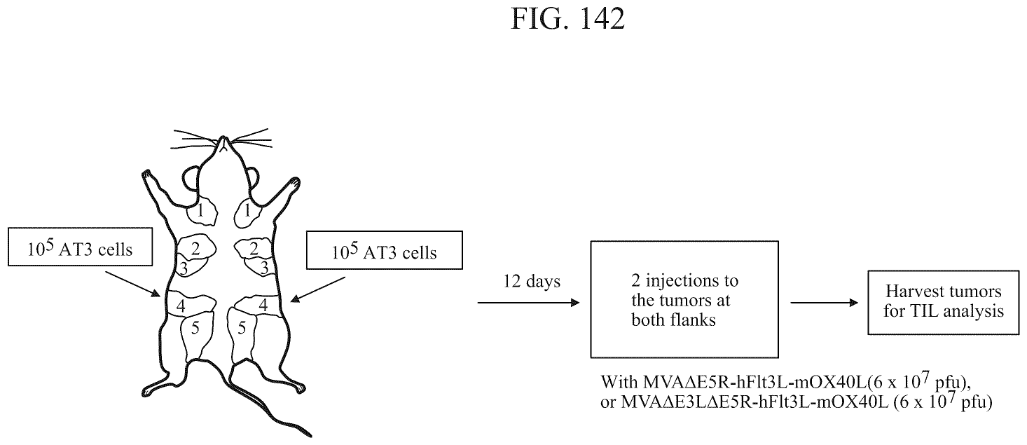

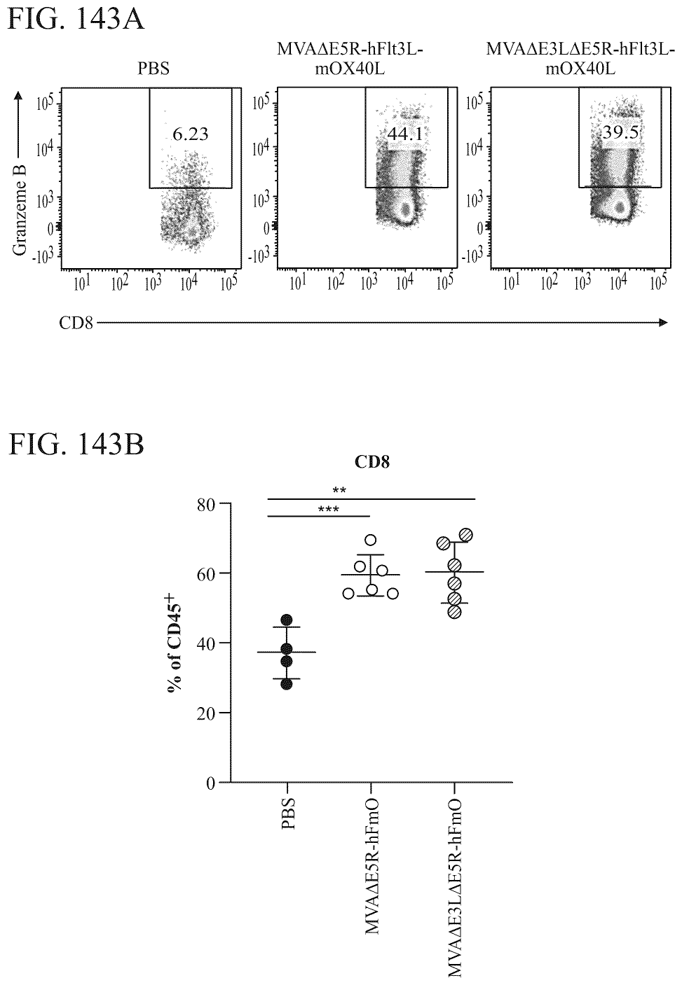

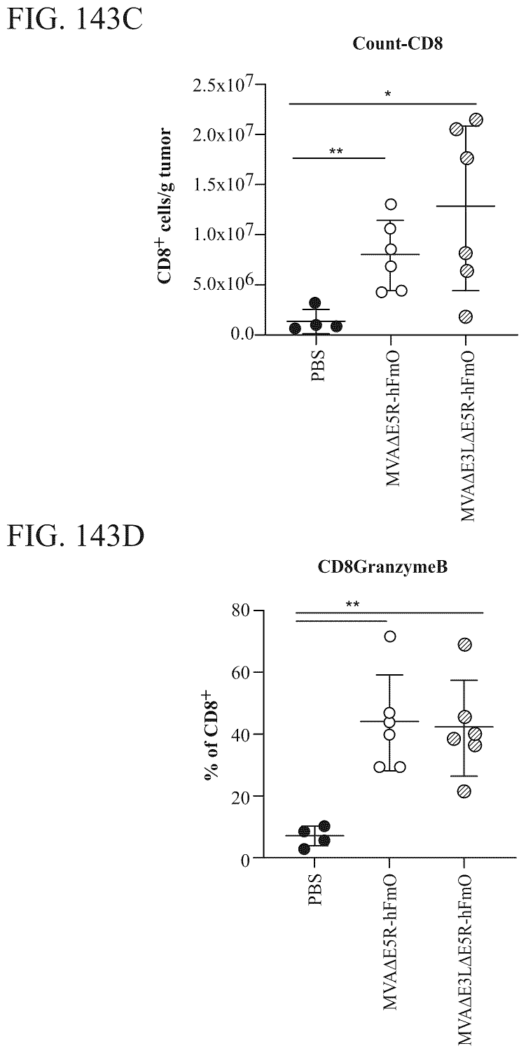

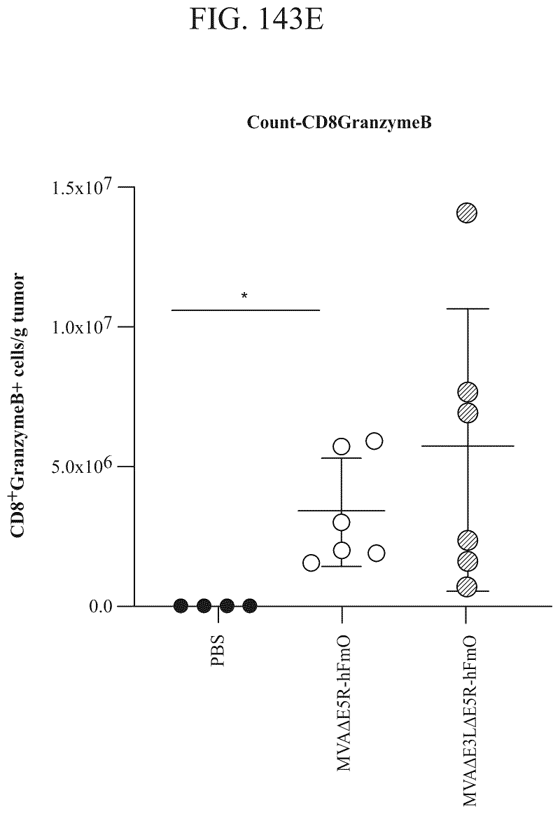

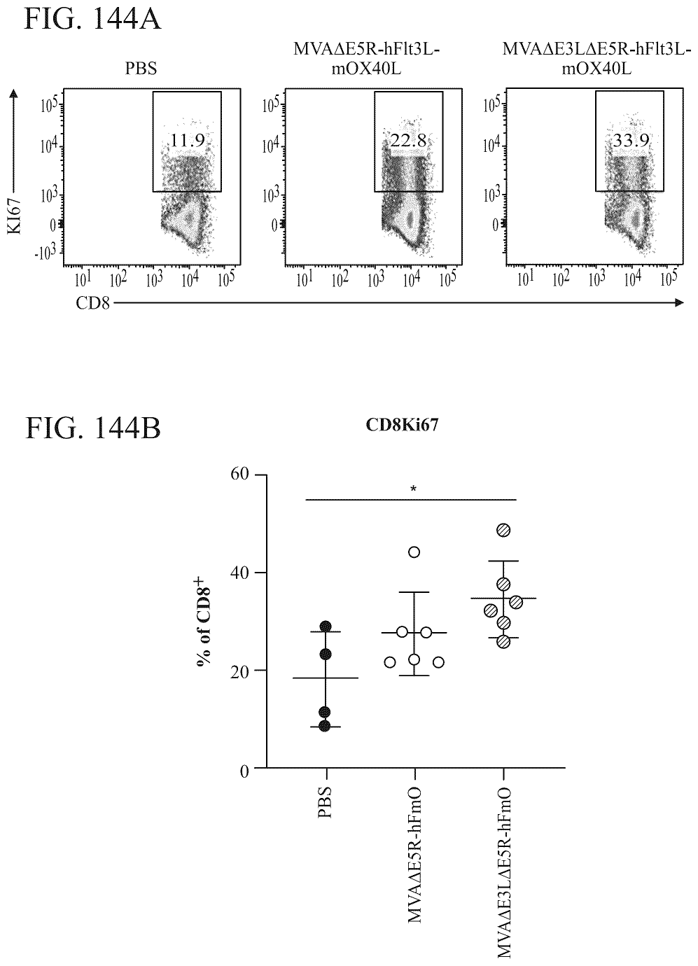

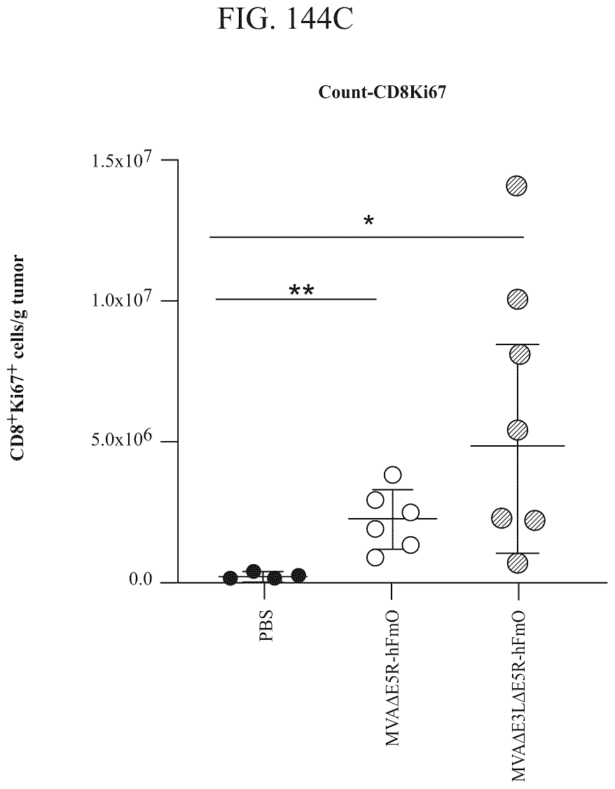

To compare the immune responses induced by IT injection of MVAΔE5R-hFlt3L-mOX40L and MVAΔE3LΔE5R-hFlt3L-mOX40L in breast tumors, a bilateral AT3 murine breast tumor implantation model was used (FIG. 142). Briefly, 1×10⁵ AT3 cells were implanted to the 4^{th} fat pad of a C57BL/6J mouse. 12 days post tumor implantation, 6×10⁷ pfu of MVAΔE5R-hFlt3L-mOX40L, or MVAΔE3LΔE5R-hFlt3L-mOX40L, or PBS were intratumorally injected twice, three days apart. Two days post second injection, the injected tumors were harvested and cells were processed for surface labeling with anti-CD3, CD45, CD4, CD8 and OX40 antibodies, and also for intracellular Granzyme B, Ki67 and FoxP3 staining. The live immune cell infiltrates in the tumors were analyzed by FACS. IT MVAΔE5R-hFlt3L-mOX40L or MVAΔE3LΔE5R-hFlt3L-mOX40L induces higher percentage of CD8⁺ T cells out of CD45⁺ cells (FIG. 143B) compared with PBS. The absolute number of CD8⁺ T cells with IT MVAΔE3LΔE5R-hFlt3L-mOX40L was higher compared with PBS and MVAΔE5R-hFlt3L-mOX40L (FIG. 143C). Both MVAΔE5R-hFlt3L-mOX40L or MVAΔE3LΔE5R-hFlt3L-mOX40L increased the percentage of Granzyme B⁺ CD8⁺ T cells compared with PBS (FIG. 143A, D). The absolute number of Granzyme B⁺ CD8⁺ T cells with IT MVAΔE3LΔE5R-hFlt3L-mOX40L was higher compared with PBS and MVAΔE5R-hFlt3L-mOX40L (FIG. 143E). IT MVAΔE3LΔE5R-hFlt3L-mOX40L also induced higher percentage and absolute number of Ki67⁺ CD8⁺ T cells (FIG. 144A-C). These results demonstrate that IT MVAΔE3LΔE5R-hFlt3L-mOX40L is more effective in activating CD8 T cells and promoting CD8⁺ T cell proliferation compared with IT MVAΔE5R-hFlt3L-mOX40L in AT3 breast cancer model. Accordingly, these results demonstrate that the viral constructs of the present technology are useful in methods for treating solid tumors.

### Example 115: Intratumoral (IT) Injection of MVAΔE3LΔE5R-hFlt3L-mOX40L Activates CD4⁺ T Cell and Reduces Regulatory T Cells in the Injected Tumors in AT3 Triple Negative Breast Cancer Model

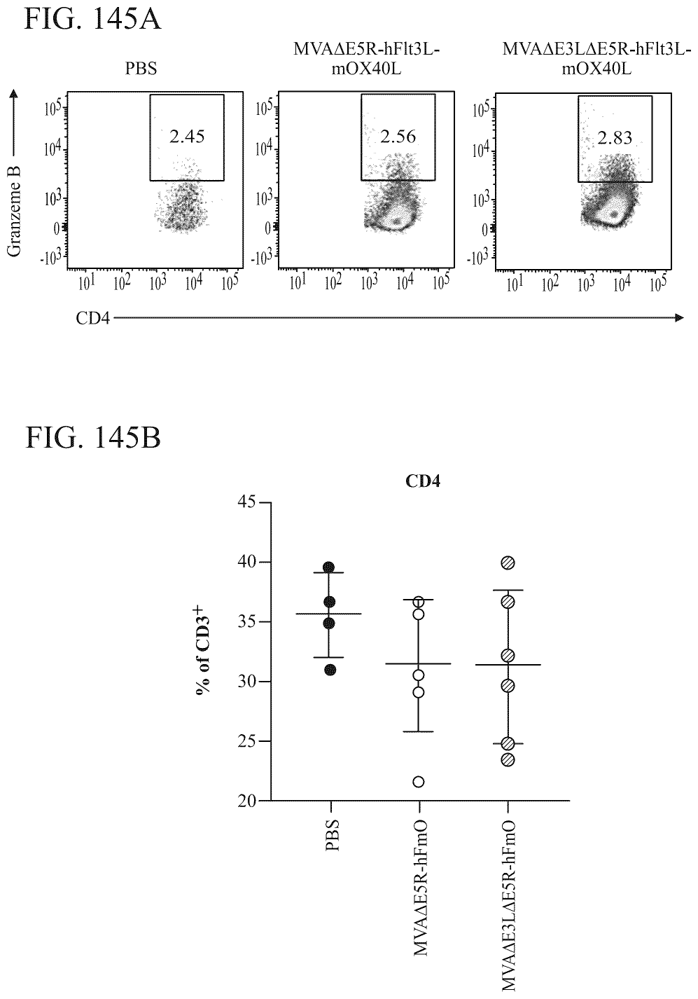

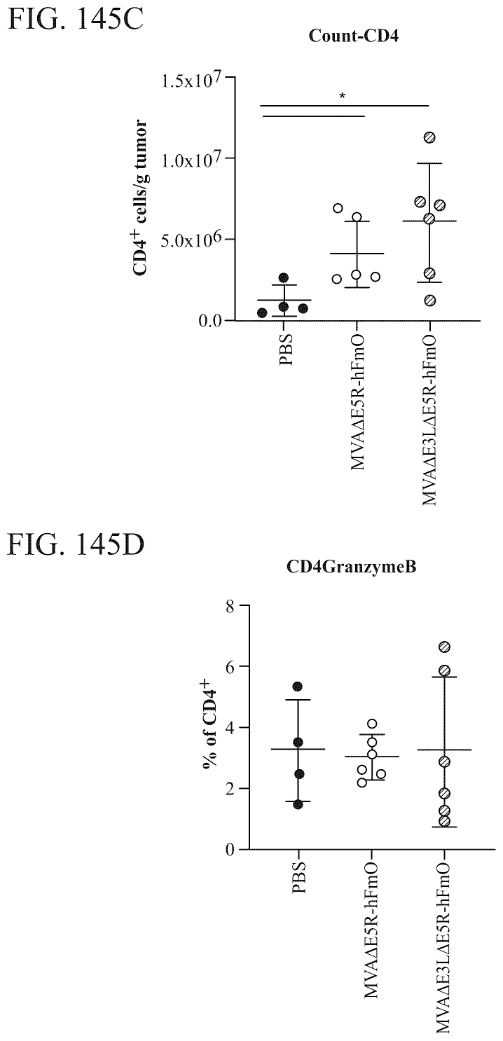

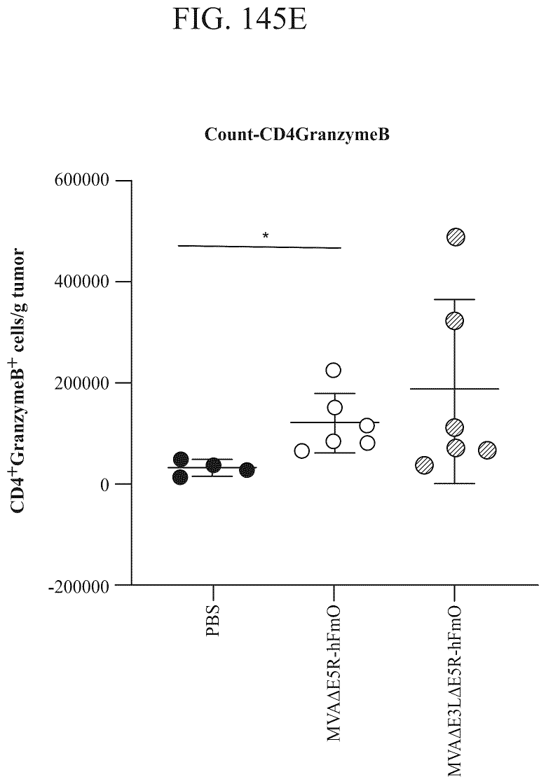

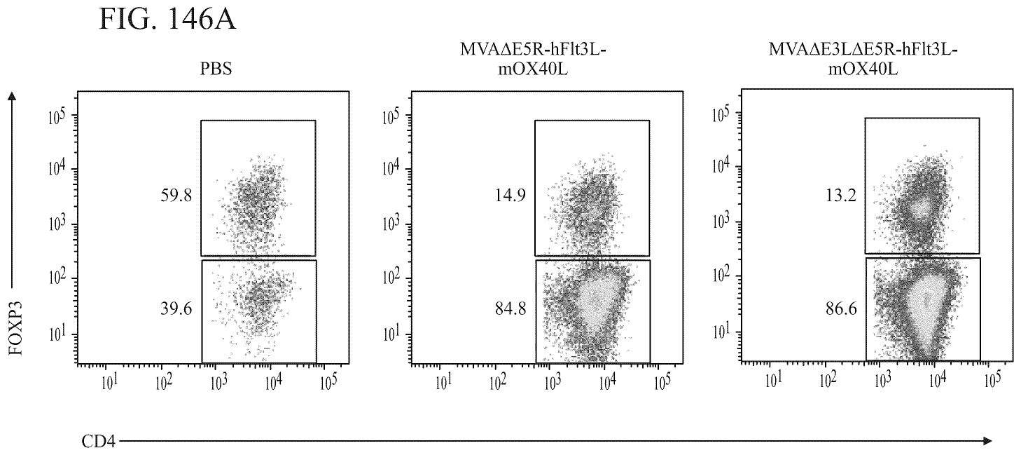

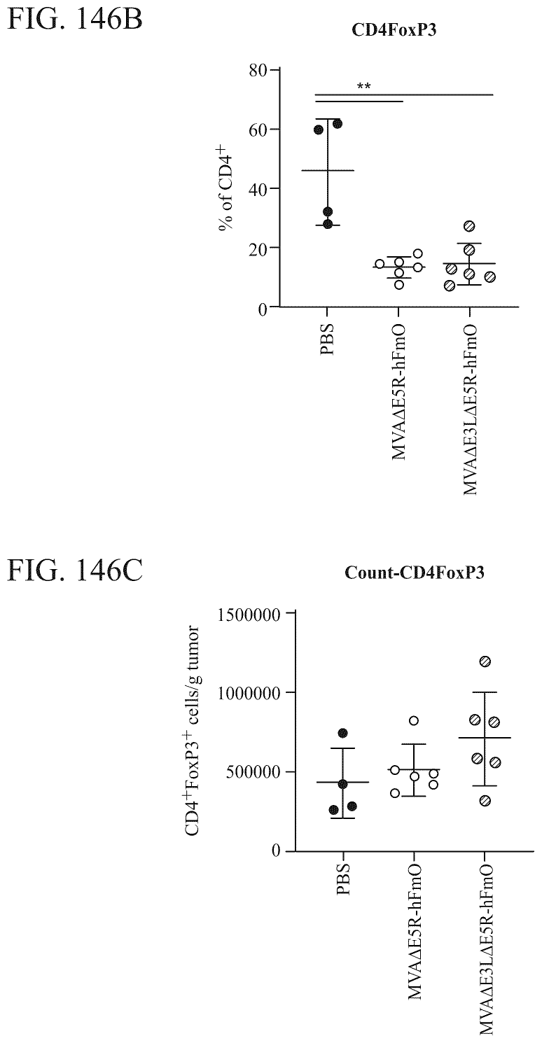

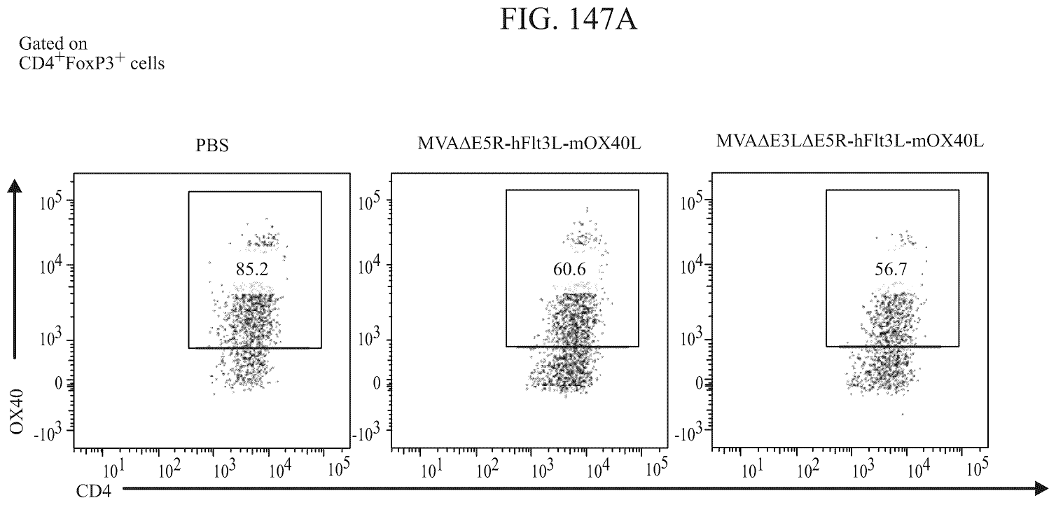

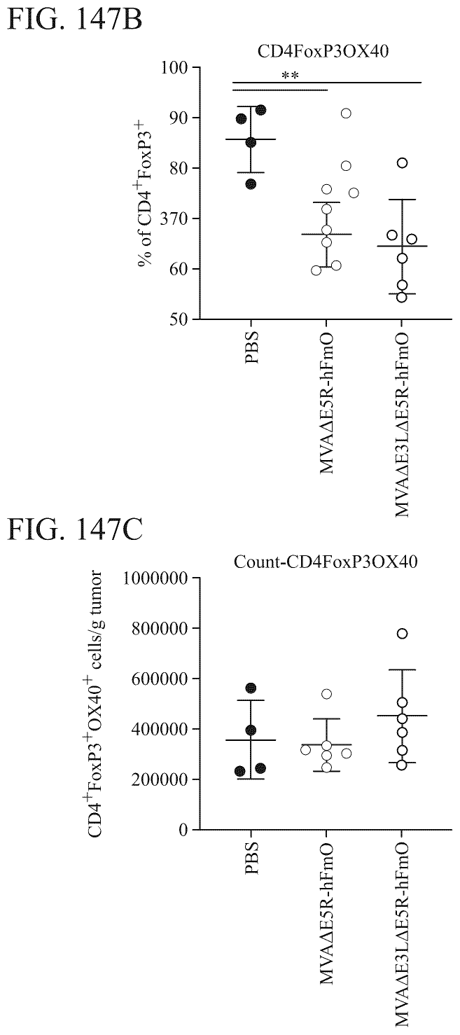

To compare the immune responses induced by IT injection of MVAΔE5R-hFlt3L-mOX40L and MVAΔE3LΔE5R-hFlt3L-mOX40L in breast tumors, a bilateral AT3 murine breast tumor implantation model was used as described in Example 114 (FIG. 142). After IT MVAΔE5R-hFlt3L-mOX40L or IT MVAΔE3LΔE5R-hFlt3L-mOX40L, the percentage of total CD4⁺ T cells out of CD3⁺ T cells did not change compared with PBS (FIG. 145B) but the absolute number of CD4⁺ T cells increased significantly after virus injection (FIG. 145C). IT MVAΔE3LΔE5R-hFlt3L-mOX40L also generated more Granzyme B⁺ CD4⁺ T cells compared with MVAΔE5R-hFlt3L-mOX40L or PBS (FIG. 145E). Both IT MVAΔE5R-hFlt3L-mOX40L or IT MVAΔE3LΔE5R-hFlt3L-mOX40L resulted in significantly reduced percentage of CD4⁺FoxP3⁺ T cells compared with PBS (FIG. 146A, 146B). The percentage of OX40⁺CD4⁺FoxP3⁺ T cells reduced after virus injection (FIG. 147A-C). These results demonstrate that IT MVAΔE3LΔE5R-hFlt3L-mOX40L induces CD4⁺ T cell activation and reduces regulatory T cells in AT3 breast cancer model. Accordingly, these results demonstrate that the recombinant poxviruses of the present technology are useful in methods for treating solid tumors.

### Example 116: Intratumoral (IT) Injection of MVAΔE3LΔE5R-hFlt3L-mOX40L Reduces Macrophages and DCs in the Injected Tumors in AT3 Triple Negative Breast Cancer Model

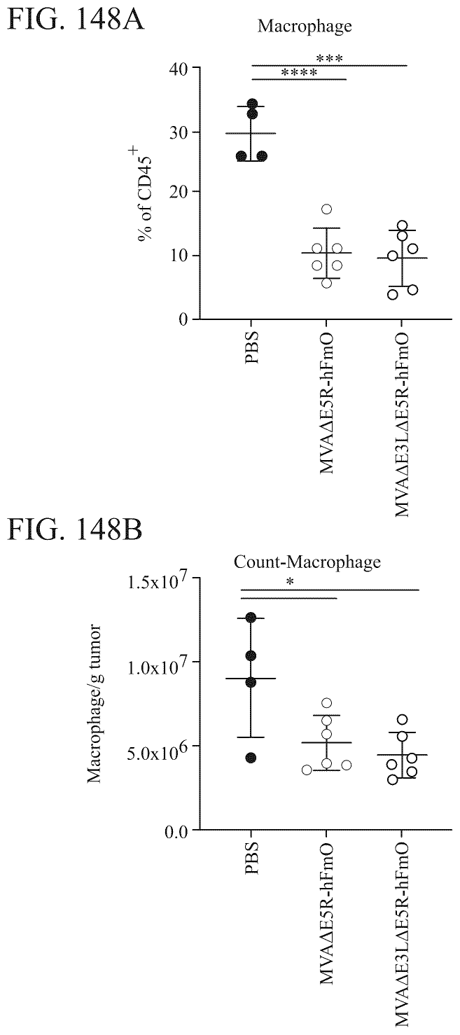

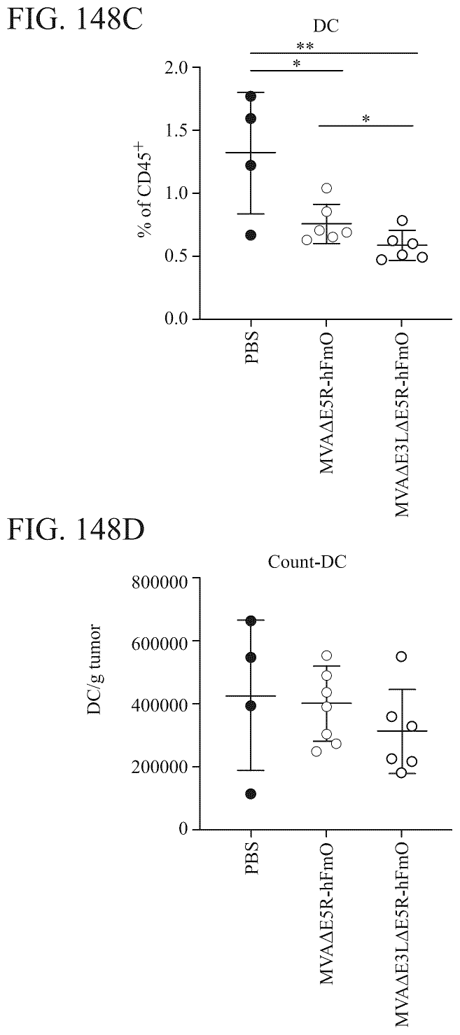

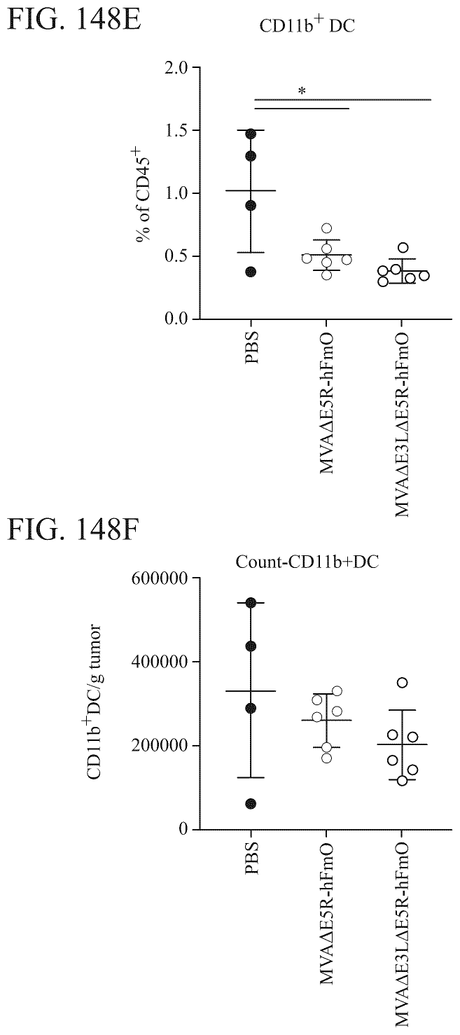

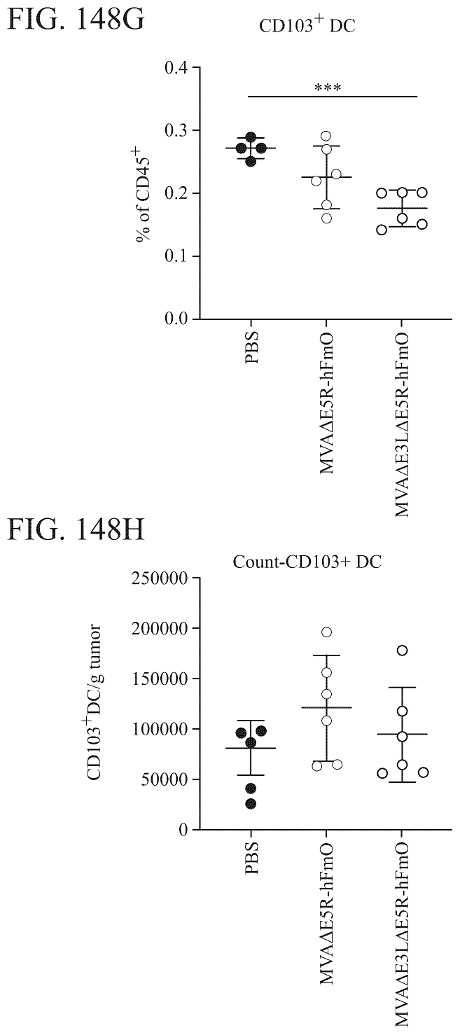

To assess whether IT MVAΔE3LΔE5R-hFlt3L-mOX40L affects myeloid cell population in breast tumors, a bilateral AT3 murine breast tumor implantation model was used as described in Example 114 (FIG. 142). Two days post second injection, the injected tumors were harvested and cells were processed for surface labeling with anti-CD3, CD19, CD49b, CD45, MHC-II, CD11c, Ly6G, F4/80, Ly6C and CD24 antibodies. The live immune cell infiltrates in the tumors were analyzed by FACS. IT MVAΔE3LΔE5R-hFlt3L-mOX40L resulted in significantly reduced percentage and number of macrophages out of total CD45⁺ cells compared with PBS (FIG. 148A, B). The percentage of dendritic cells was reduced with IT MVAΔE3LΔE5R-hFlt3L-mOX40L and IT MVAΔE5R-hFlt3L-mOX40L (FIG. 148C), with decreased percentage of both CD11b⁺ and CD103⁺ DCs (FIG. 148E-H). These results demonstrate that IT MVAΔE3LΔE5R-hFlt3L-mOX40L reduces macrophages and DCs in the injected tumors in AT3 breast cancer model. Accordingly, these results demonstrate that the recombinant poxviruses of the present technology are useful in methods for treating solid tumors.

### Example 117: Spontaneous Breast Cancers are Responsive to the Combination Therapy with IT MVAΔE3LΔE5R-hFlt3L-mOX40L and Systemic Delivery of Anti-PD-L1 and Anti-CTLA-4 Antibodies

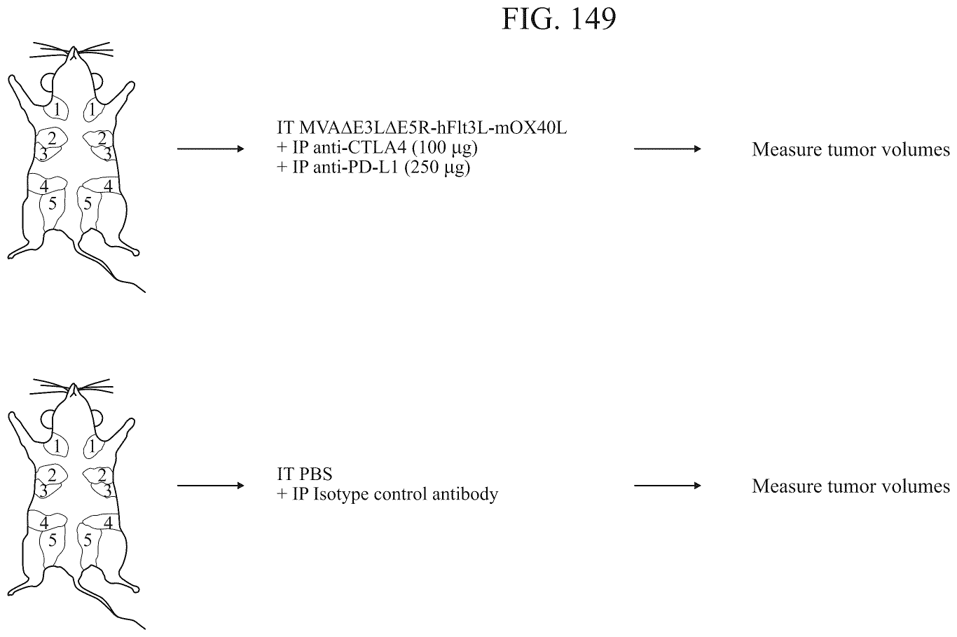

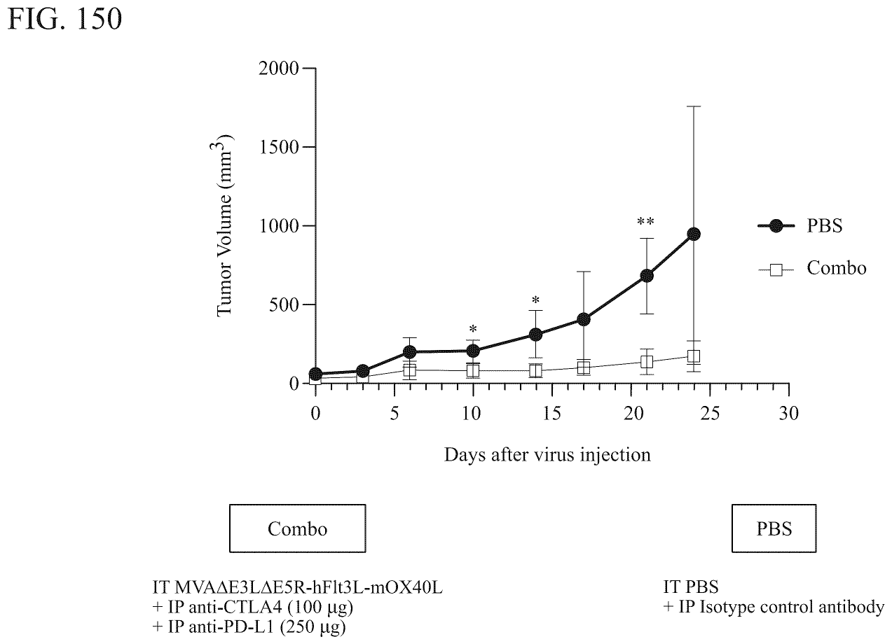

To evaluate therapeutic efficacy of IT MVAΔE3LΔE5R-hFlt3L-mOX40L in combination with anti-PD-L1 and anti-CTLA-4 antibody in triple negative breast cancers, a MMTV-PyMT spontaneous breast cancer model was used. After the first tumor became palpable, injection of MVAΔE5R-hFlt3L-mOX40L to tumors was started. 250 μg Anti-PD-L1 and 100 μg anti-CTLA-4 antibodies were given intraperitoneally to each mouse. Tumor sizes were measured twice a week (FIG. 149). The combo treatment resulted in delayed tumor growth compared with PBS control group week (FIG. 150). This result demonstrates that spontaneous breast cancers are responsive to the combination therapy with IT MVAΔE3LΔE5R-hFlt3L-mOX40L and systemic delivery of anti-PD-L1 and anti-CTLA-4 antibodies. Accordingly, these results demonstrate that the recombinant poxvirus compositions of the present technology are useful in methods for treating solid tumors.

### Example 118: Generation of MVAΔE5R hFlt3L-mOX40LΔC11R Recombinant Virus

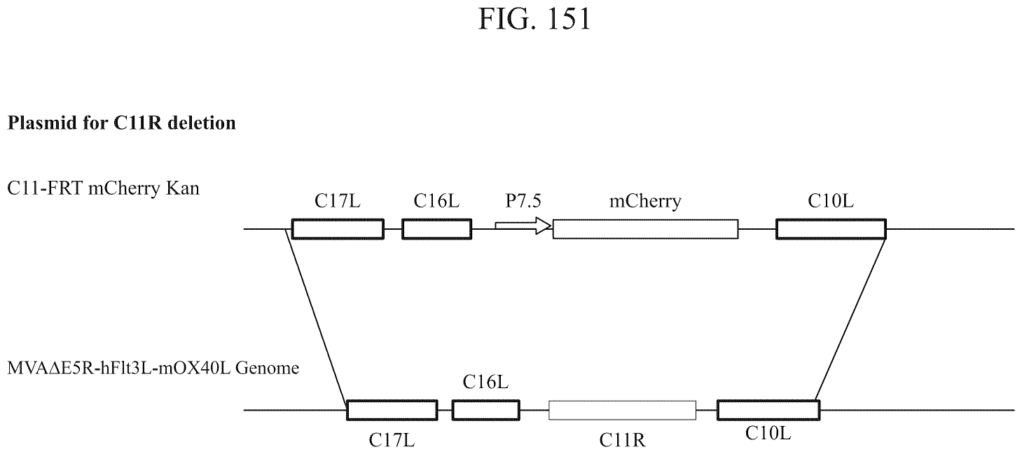

The vaccinia C11R encodes vaccinia growth factor. The WT vaccinia (Western Reserve) genome has two copies, whereas the MVA genome has one copy. The C11 gene was identified as one of the eight vaccinia early genes involved in inhibiting the cGAS/STING pathway in a dual-luciferase screening assay (FIGS. 19 and 20). To test whether deletion of the C11R gene from MVAΔE5R-hFlt3L-mOX40L enhances type I IFN induction capacity of the virus, a recombinant virus MVAΔE5R-hFlt3L-mOX40LΔC11R was generated through homologous recombination at the C17L/C16L and C10L loci of the MVAΔE5R-hFlt3L-mOX40L genome (FIG. 151).

### Example 119: MVAΔE5R hFlt3L-mOX40LΔC11R Induces Higher Levels of IFNB Gene Expression and IFN-β Protein Secretion in BMDCs Compared with MVAΔE5R hFlt3L-mOX40L

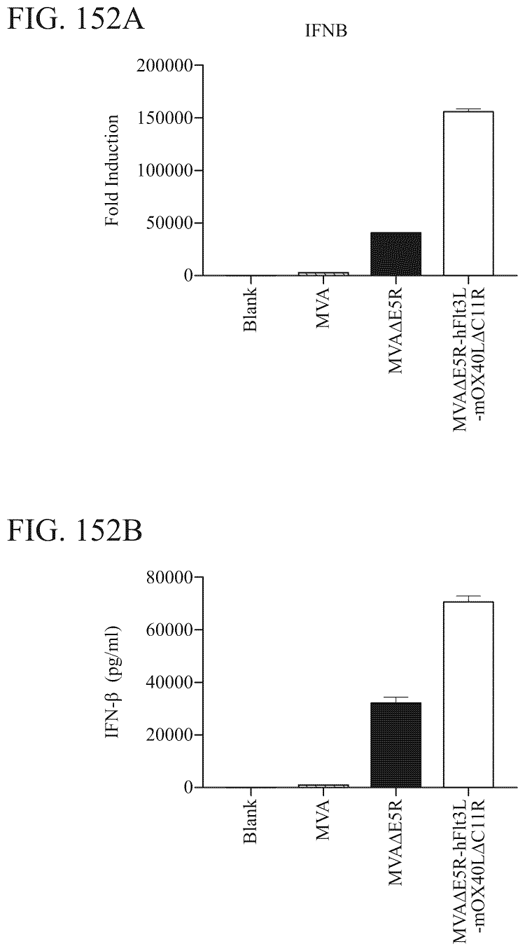

To test whether MVAΔE5R-hFlt3L-mOX40LΔC11R induces higher levels of type I IFN compared with MVAΔE5R, BMDCs (1×10⁶) from WT C57BL/6J mice were infected with either MVAΔE5R-hFlt3L-mOX40LΔC11R, or MVAΔE5R, or MVA at a MOI of 10. Cells were collected at 6 h post infection. Supernatants were collected at 19 h post infection. IFNB gene expression was determined by RT-PCR. The results show that MVAΔE5R-hFlt3L-mOX40LΔC11R induces higher levels of IFNB gene expression compared with MVAΔE5R (FIG. 152A).

IFN-β protein levels in the supernatants were determined by ELISA (FIG. 152B). The results show that MVAΔE5R-hFlt3L-mOX40LΔC11R infection of BMDCs induce higher levels of IFN-β protein secretion compared with MVAΔE5R. These results indicate that removing the C11R gene from MVAΔE5R-hFlt3L-mOX40L further improves IFN induction capacity of the viruses.

### Example 120: Generation of MVAΔWR199 and MVAΔE5R hFlt3L-mOX40LΔWR199 Recombinant Viruses

The vaccinia WR199 gene encodes a 68-Kda ankyrin-repeat protein, and is one of the eight vaccinia early genes identified in the screening for inhibitors of cGAS/STING pathway (FIG. 19). To test whether deletion of the WR199 gene from MVA or MVAΔE5R-hFlt3L-mOX40L enhances type I IFN induction capacity of the viruses respectively, recombinant viruses MVAΔWR199 and MVAΔE5R-hFlt3L-mOX40LΔWR199 were generated through homologous recombination at the B17L and B19R loci of the MVA and MVAΔE5R-hFlt3L-mOX40L genome (FIG. 153). FRT sites were placed at the flanking region of the gene encoding mcherry to facilitate fluorescent color removal for further engineering of the virus.

### Example 121: Deletion of the WR199 Gene from MVA or MVAΔE5R hFlt3L-mOX40L Improves IFNB Gene Induction Capacity of the Viruses in BMDCs

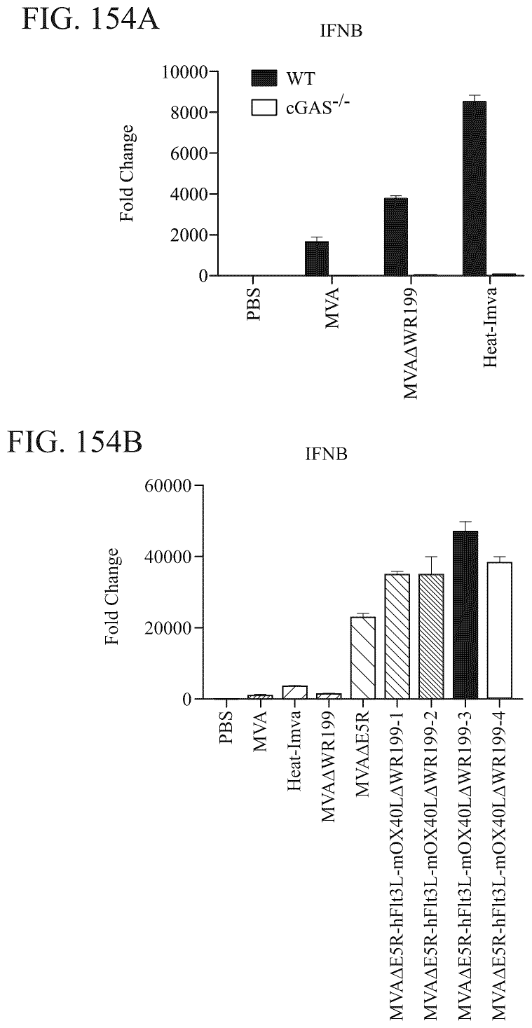

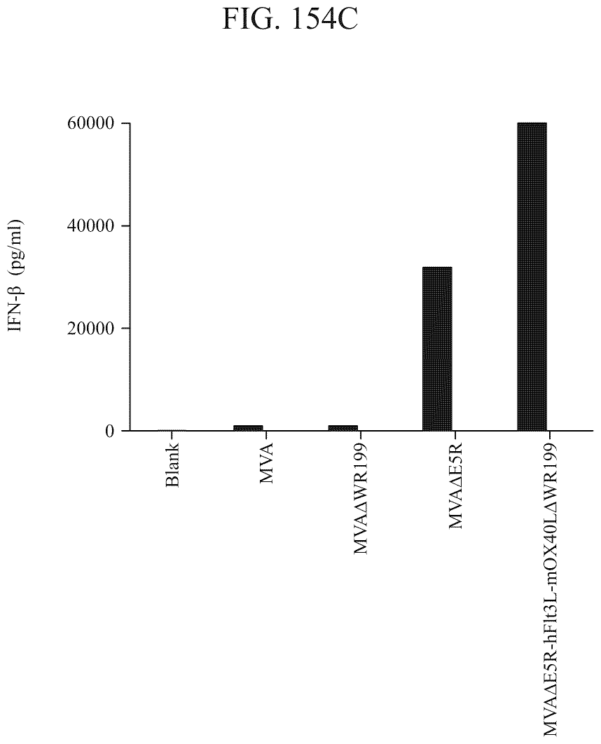

To test whether deleting the WR199 gene from MVA or MVAΔE5R-hFlt3L-mOX40L induces higher levels of type I IFN, BMDCs (1×10⁶) from WT and cGAS^{-/-} C57BL/6J mice were infected with either MVA or MVAΔWR199 at a MOI of 10 or with Heat-iMVA at an equivalent amount. Cells were collected at 6 h post infection. Supernatants were collected at 19 h post infection. IFNB gene expression was determined by RT-PCR. The results show that MVAΔWR199 induces higher levels of IFNB gene expression compared with MVA, but lower levels of IFNB compared with Heat-iMVA in BMDCs (FIG. 154A). In cGAS^{-/-} BMDCs, MVAΔWR199-induced IFNB gene induction is completely lost (FIG. 154A).

Four isolated clones of MVAΔE5R-hFlt3L-mOX40LΔWR199. BMDCs were generated and cells were infected with either one of the four clones, MVAΔWR199, or MVAΔE5R. RT-PCR analysis showed that MVAΔE5R-hFlt3L-mOX40LΔWR199 induces higher levels of IFNB gene expression compared with MVAΔE5R or MVAΔWR199 (FIG. 154B).

IFN-β protein levels in the supernatants were determined by ELISA (FIG. 154C). The results show that MVAΔE5R-hFlt3L-mOX40LΔWR199 infection of BMDCs induces higher levels of IFN-β protein secretion compared with MVAΔE5R or MVAΔWR199. These results indicate that removing the WR199 gene from either MVA or MVAΔE5R-hFlt3L-mOX40L further improves IFN induction capacity of the viruses.

### Example 122: Generation of Recombinant Vaccinia Virus with Deletion of B2R (VACVΔB2R)

It was recently reported that the vaccinia B2R gene encodes a nuclease that degrades 2′,3′-cyclic GMP-AMP (cGAMP), which contributes to immune evasion of the cytosolic DNA-sensing pathway mediated by cGAS (Eaglesham et al., 2019). This gene is highly conserved among the poxvirus family. However, the B2R gene in MVA is truncated and the protein is inactive. A mutant vaccinia with B2R deletion from vTF7-3 Western Reserve strain, in which the vaccinia thymidine kinase gene (TK) was generated and was found to be more attenuated than the parental virus in a skin scarification model. To test the effect of B2R gene deletion on viral virulence independent of TK deletion, a VACVΔB2R mutant virus was generated and evaluated the virulence of the virus in an intranasal infection model.

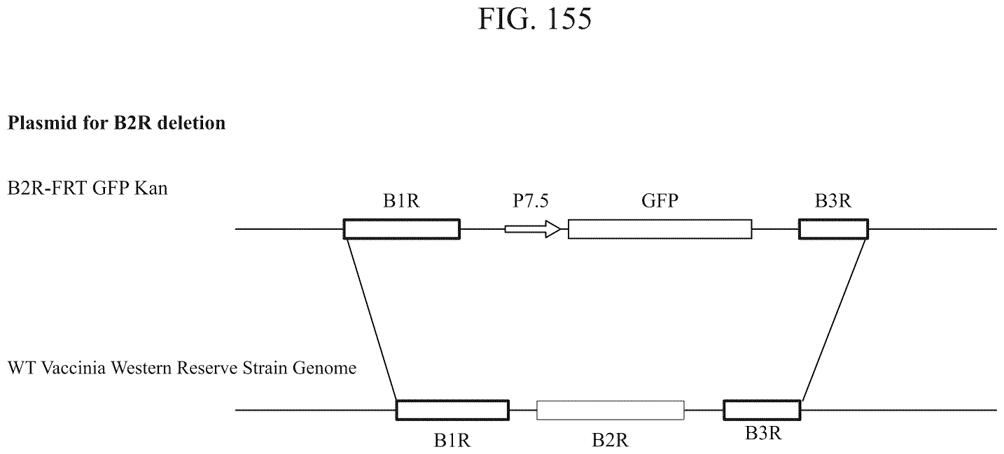

Briefly, pB2R-FRT GFP vector were used to insert GFP under the control of the vaccinia P7.5 promoter into the B2R locus of vaccinia virus (VACV; Western Reserve strain). The expression cassette is flanked by partial sequence of the BlR and B3R genes on either side (FIG. 155). BSC40 cells were infected with WT vaccinia virus at a MOI of 0.05 for 1 h, and then were transfected with the plasmid DNA described above. The infected cells were collected at 48 h. Recombinant viruses were identified by their green fluorescence with the insertion of GFP into the B2R locus. The positive clones were plaque purified 4-5 times on BSC40 cells. PCR analysis were performed to confirm that the recombinant virus VACVΔB2R has lost the B2R gene.

### Example 123: The Vaccinia B2R Gene Encodes a Virulence Factor

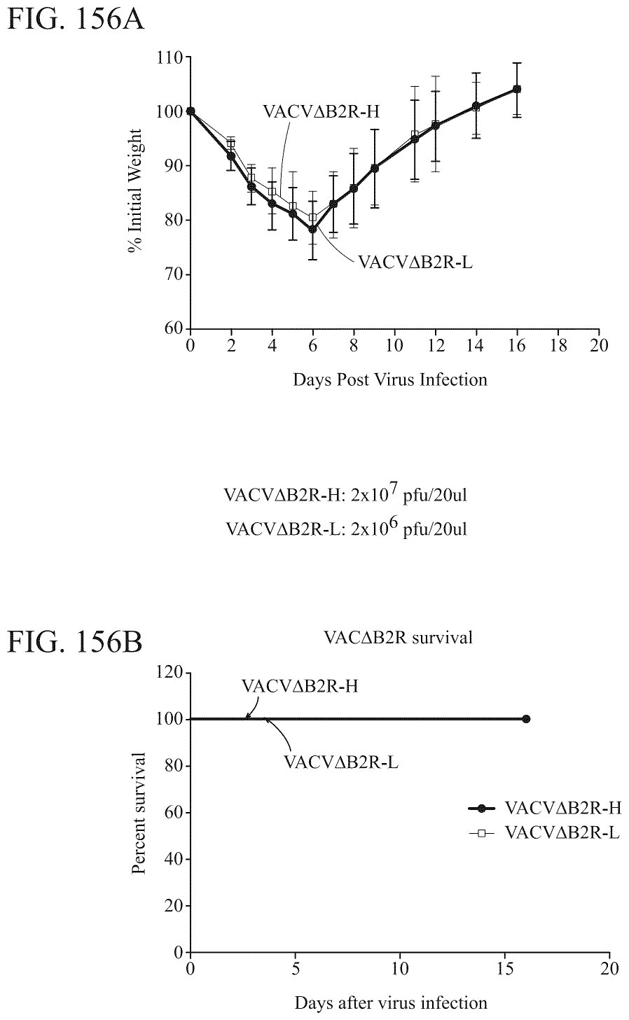

To determine whether the vaccinia virus B2R gene contributes to virulence in an intranasal infection model, groups of 6-week-old WT female C57BL/6J mice were intranasally infected with two different doses (2×10⁷ or 2×10⁶ pfu) of VAVCΔB2R. Weight loss and survival of C57BL/6J mice after intranasal infection with VACVΔB2R were monitored. Infection of VACVΔB2R at either dose resulted in maximal weight loss (around 20% of the initial weight) around day 6 post infection. The mice regained their weight at day 13 post infection and all of them survived (FIGS. 156A and B). These results suggested that VAVCΔB2R is highly attenuated in the intranasal infection model compared with WT VACV, which has a LD₅₀ of 2×10⁵ pfu.

### Example 124: Generation of Recombinant Vaccinia Viruses VACVΔE5RΔB2R and VACVΔE3L83NΔE5RΔB2R

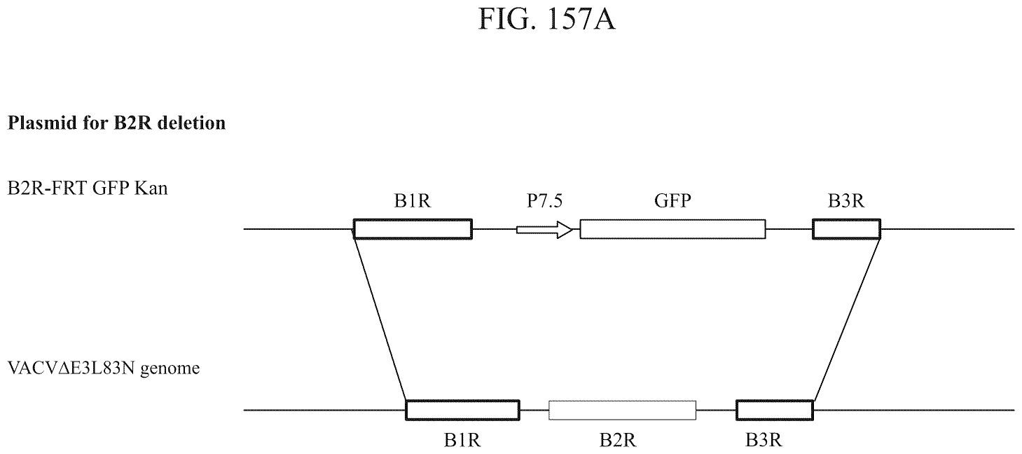

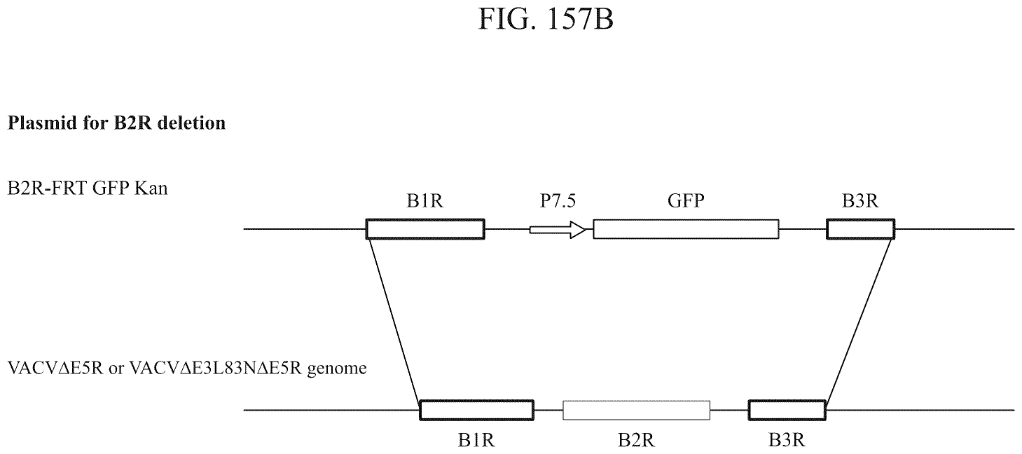

To test the effect of compound deletions of both B2R and E5R genes from either WT VACV background or VACVΔE3L83N, which lacks the DNA fragment encoding the N-terminal 83 amino acid Z-DNA binding domain, VACVΔE5RΔB2R and VACVΔE3L83NΔE5RΔB2R were generated. Briefly, pB2R-FRT GFP vector were used to insert GFP under the control of the vaccinia P7.5 promoter into the B2R locus of mutant vaccinia virus VACVΔE5R (FIG. 157A), or VACVΔE3L83NΔE5R (FIG. 157B), which was generated by deleting E5R gene from VACVΔE3L83N virus. The expression cassette is flanked by partial sequence of the B1R and B3R genes on either side (FIG. 157).

BSC40 cells were infected with either VACVΔE5R or VACVΔE3L83NΔE5R at a MOI of 0.05 for 1 h, and then were transfected with the plasmid DNAs described above. The infected cells were collected at 48 h. Recombinant viruses were identified by their green fluorescence with the insertion of GFP into the B2R locus. The positive clones were then plaque purified 4-5 times on BSC40 cells. PCR analysis were performed to confirm that recombinant viruses VACVΔE5RΔB2R and VACVΔE3L83NΔE5RΔB2R have lost the B2R gene.

### Example 125: Recombinant Vaccinia Viruses VACVΔE5RΔB2R and VACVΔE3L83NΔE5RΔB2R are Highly Attenuated in a Murine Intranasal Infection Model

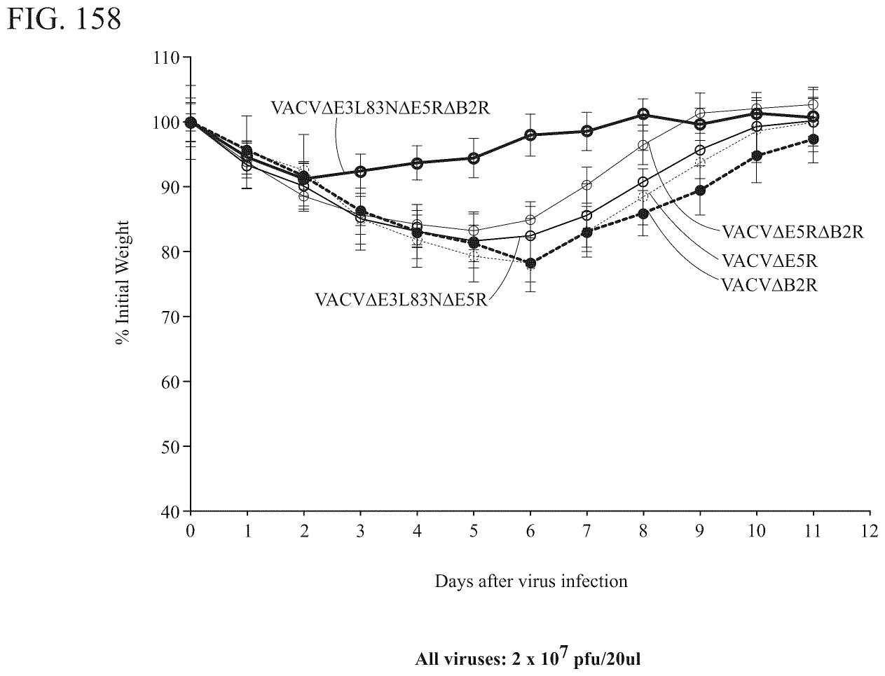

Intranasal infection of 6-week-old WT female C57BL/6J mice was performed with 2×10⁷ pfu of VACVΔE5R, VACVΔB2R, VACVΔE3L83NΔE5R, VACVΔE5RΔB2R, or VACVΔE3L83NΔE5RΔB2R. Weight loss and survival of the mice were monitored (FIG. 158). Infection with VACVΔE3L83NΔE5RΔB2R resulted the least weight loss in these mice, with maximum weight loss around 10% of the initial weight (FIG. 158). Infection with VACVΔE5RΔB2R also resulted less weight loss compared with the VACVΔE5R or VACVΔB2R (FIG. 158). All of mice survived the infection. These results indicate that compound deletion of B2R and E5R or B2R, E5R, and E3L83N results in further attenuation of the viruses.

### Example 126: VACVΔE5RΔB2R or VACVΔE3L83NΔE5RΔB2R Infection of Bone Marrow-Derived Dendritic Cells (BMDCs) Induces Higher Levels of Type I IFNB Gene Expression and IFN-β Protein Secretion than VACVΔB2R, or VACVΔE5R

Vaccinia E5R gene expression leads to cGAS degradation (Example 63 and 64). The vaccinia B2R gene encodes a nuclease that degrades cGAMP, which is a product of cGAS. Without being bound by theory, it is hypothesized that double deletion of B2R and E5R gene from the vaccinia genome can lead enhanced induction of IFNB gene expression and IFN-β protein secretion in infected BMDCs.

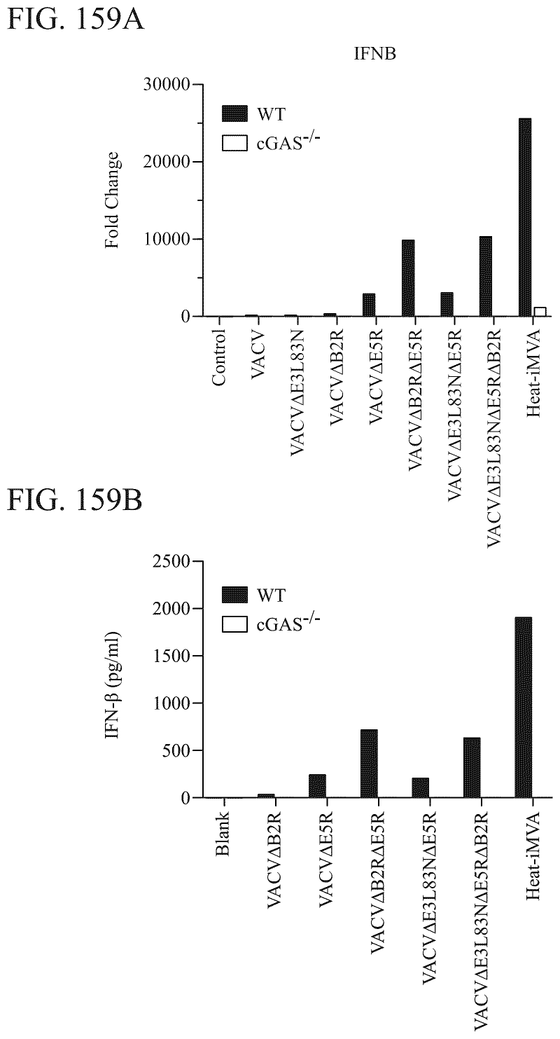

To test that, WT and cGAS^{-/-} BMDCs were infected with VACVΔE3L83NΔE5R, VACVΔE5RΔB2R, VACVΔE3L83NΔE5RΔB2R, VACVΔE3L83N, VACVΔB2R, or VACVΔE5R at a MOI of 10 or with an equivalent amount of Heat-iMVA. Cells were collected at 6 h post infection. RNAs were extracted. The IFNB gene expression levels were determined by quantitative PCR analyses. Supernatants were collected at 24 h post infection and IFN-β levels were measured by ELISA. RT-PCR results showed that whereas WT VACV infection of BMDCs induced 225-fold higher levels of IFNB gene expression compared with no-treatment control, and VACVΔE3L83N, VACVΔB2R, or VACVΔE5R, VACVΔE3L83NΔE5R infection induced 223-, 410-, 3054-, 3117-fold higher levels of IFNB gene expression compared with no-treatment control, respectively. VACVΔE5RΔB2R or VACVΔE3L83NΔE5RΔB2R infection induced 9970- and 10383-fold higher levels of IFNB gene expression compared with no-treatment control (FIG. 159A). ELISA results showed that VACVΔB2R, VACVΔE5R, or VACVΔE3L83NΔE5R infection of BMDCs induced IFN-β protein secretion from BMDCs resulting in IFN-β levels in the supernatants at 22, 232, and 189 μg/ml respectively. VACVΔE5RΔB2R or VACVΔE3L83NΔE5RΔB2R infection induced higher level of IFN-β secretion with the IFN-β at 700 and 763 μg/ml in the supernatants (FIG. 159B). In the cGAS^{-/-} BMDCs, the attenuated vaccinia fails to induce IFNB gene expression or IFN-β protein secretion (FIGS. 159A and 159B).

These results indicate that the combined deletion of B2R and E5R induces higher levels of IFNB gene expression and IFN-β protein secretion from BMDCs compared with single B2R or E5R deletion. And the induction of IFNB gene expression and IFN-β protein secretion from BMDCs infected with the attenuated mutant vaccinia is recombinant poxviruses of the present technology are useful in methods for treating solid tumors.

### Example 127: VACVΔE5RΔB2R or VACVΔE3L83NΔE5RΔB2R Infection of Bone Marrow-Derived Dendritic Cells (BMDCs) Induces Higher Levels of Phosphorylation of STING, TBK1 and IRF3 Compared with VACVΔB2R, or VACVΔE5R

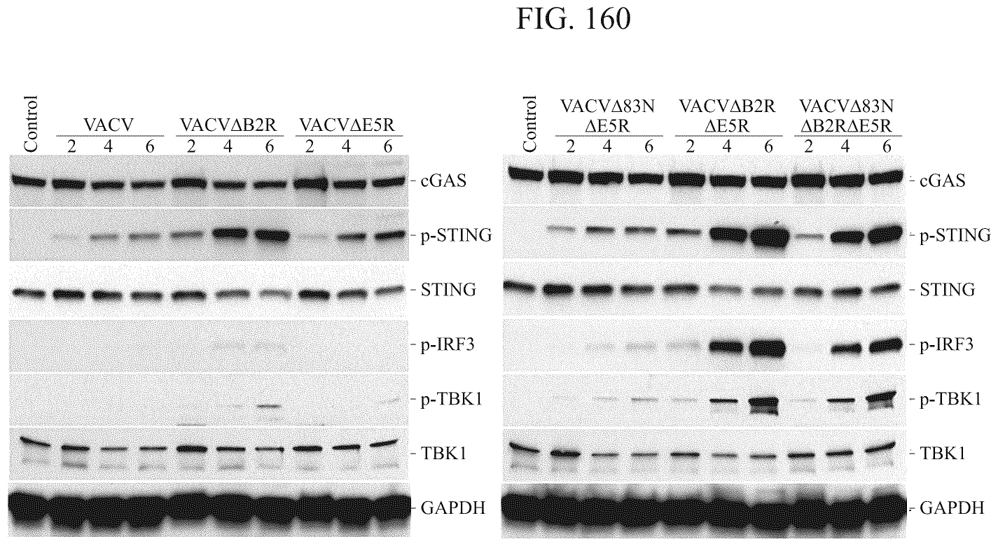

To test whether the double deletions of B2R and E5R from WT VACV genome or the triple deletions of E3L83N, B2R, and E5R from WT VACV genome would induce stronger activation of the cGAS-STING signaling pathway, BMDCs were infected with VACV, VACVΔB2R, VACVΔE5R, VACVΔE3L83NΔE5R, VACVΔE5RΔB2R, or VACVΔE3L83NΔE5RΔB2R at a MOI of 10. Cell lysates were collected at 2, 4, and 6 h post infection. Proteins were separated in SDS-PAGE gel, and were blotted with antibodies against phosphorylated STING, TKB1, and IRF3. VACVΔE5RΔB2R or VACVΔE3L83NΔE5RΔB2R induced higher levels of phosphorylation of STING, TBK1, and IRF3 in infected BMDCs compared with VACVΔB2R or VACVΔE5R (FIG. 160). These results indicate that the double deletions of B2R and E5R from WT VACV genome or the triple deletions of E3L83N, B2R, and E5R from WT VACV genome results in stronger activation of the cGAS/STING/TBK1/IRF3 signaling pathway.

### Example 128: Generation of the Recombinant Vaccinia Viruses VACVΔE3L83N-ΔTK-Anti-muCTLA-4-ΔE5R-hFlt3L-mOX40L-mIL-12 (OV-VACVΔE5R) or VACVΔE3L83N-ΔTK-Anti-muCTLA-4-ΔE5R-hFlt3L-mOX40L-mIL-12-ΔB2R (OV-VACVΔB2R)

This example describes the generation of the recombinant VACVΔE3L83N-ΔTK-anti-muCTLA-4-ΔE5R-hFlt3L-mOX40L-mIL-12 or VACVΔE3L83N-ΔTK-anti-muCTLA-4-ΔE5R-hFlt3L-mOX40L-mIL-12-ΔB2R viruses.

FIG. 161 shows the stepwise strategy to generate recombinant VACVΔE3L83NΔTKΔE5R virus expressing anti-muCTLA-4, hFlt3L, mOX40L and mIL12 proteins through homologous recombination first at the TK locus and then at the E5R locus of the VACVΔE3L83N genome. pCB-anti-muCTLA-4 gpt vector was used to insert a single expression cassette to express the anti-muCTLA-4 antibody heavy and light chains under the control of the vaccinia virus synthetic early and late promoter (PsE/L). Homologous recombination that occurred at the TK-L and TK-R sites results in the insertion of expression cassette of anti-CTLA-4 antibody into TK locus on VACVΔE3L83N genome, generating the recombinant virus VACVΔE3L83N-ΔTK-anti-muCTLA-4.

pUC57-hFlt3L-mOX40L-mIL12 mCherry vector was used to insert a single expression cassette designed to express both hFlt3L-mOX40L fusion protein and mIL12 protein separately using the vaccinia viral synthetic early and late promoter (PsE/L) in opposite directions. The coding sequence of the hFlt3L-mOX40L was separated by a cassette including a furin cleavage site followed by a Pep2A sequence. Homologous recombination at the E4L and E6R loci resulted in the insertion of the expression cassette for hFlt3L-mOX40L and mIL12 into the E5L locus of VACVΔE3L83N-ΔTK-anti-muCTLA-4 virus. BSC40 cells were infected with VACVΔE3L83N at a MOI of 0.05 for 1 h, and then were transfected with the plasmid pCB-anti-muCTLA-4 gpt. The infected cells were collected at 48 h. Recombinant viruses were selected through further culturing in selection medium including MPA, xanthine and hypoxanthine, and plaque purified. PCR analysis was performed to verify the insertion of anti-muCTLA-4 gene into the TK locus.

For inserting hFlt3L-mOX40L-mIL12 expression cassette into the E5R locus of VACVΔE3L83N-ΔTK-anti-muCTLA-4 virus, BSC40 cells were infected with VACVΔE3L83N-ΔTK-anti-muCTLA-4 at a MOI of 0.05 for 1 h, and then were transfected with the plasmid pUC57-hFlt3L-mOX40L-mIL12 mCherry. The infected cells were collected at 48 h. Recombinant viruses were isolated through plaque purification for at least 4-5 rounds by selecting mCherry positive plaques. PCR analysis was performed to verify the insertion of hFlt3L-mOX40L-mIL12gene into the E5R locus.

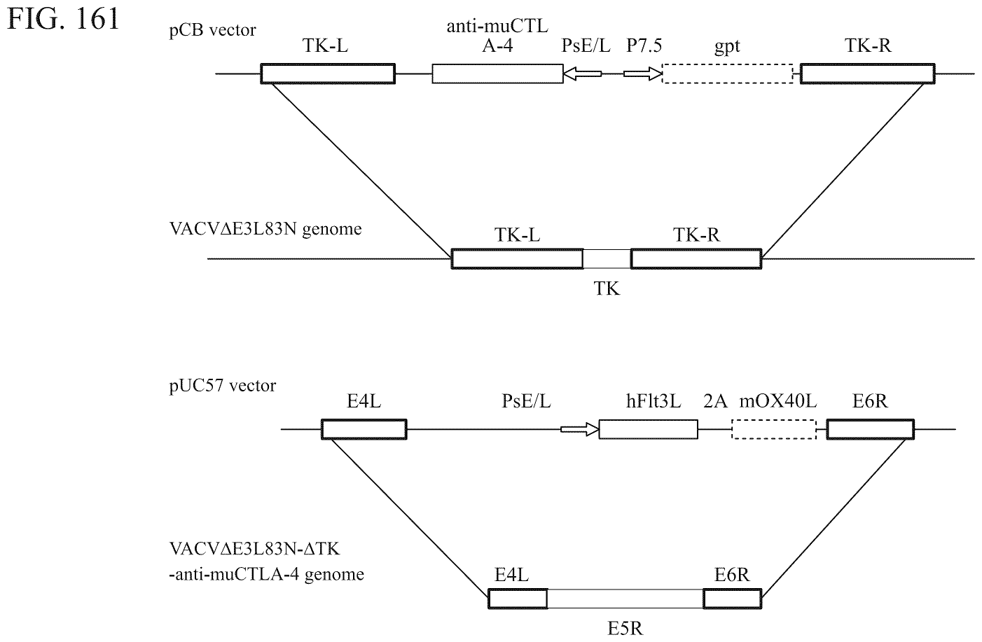

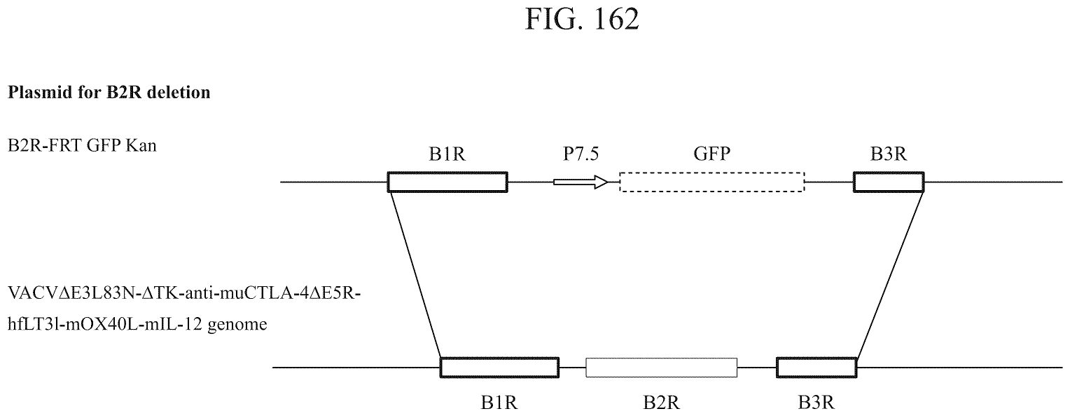

FIG. 162 shows the generation of VACVΔE3L83N-ΔTK-anti-muCTLA-4-ΔE5R-hFlt3L-mOX40L-mIL-12-ΔB2R virus through the deletion of B2R gene from VACVΔE3L83N-ΔTK-anti-muCTLA-4-ΔE5R-hFlt3L-mOX40L-mIL-12 virus. pB2R-FRT GFP vector were used to insert GFP under the control of the vaccinia P7.5 promoter into the B2R locus of recombinant virus VACVΔE3L83N-ΔTK-anti-muCTLA-4-ΔE5R-hFlt3L-mOX40L-mIL-12. BSC40 cells were infected with VACVΔE3L83N-ΔTK-anti-muCTLA-4-ΔE5R-hFlt3L-mOX40L-mIL-12 virus at a MOI of 0.05 for 1 h, and then were transfected with the plasmid DNA described above. The infected cells were collected at 48 h. Recombinant viruses were identified by their green fluorescence with the insertion of GFP into the B2R loci and by mCherry in the parental virus. The positive clones were then plaque purified 4-5 times on BSC40 cells. PCR analysis were performed to confirm that recombinant virus VACVΔE3L83N-ΔTK-anti-muCTLA-4-ΔE5R-hFlt3L-mOX40L-mIL-12 virus has lost the B2R gene.

### Example 129: VACVΔE3L83N-ΔTK-Anti-muCTLA-4-ΔE5R-hFlt3L-mOX40L-mIL-12 and VACVΔE3L83N-ΔTK-Anti-muCTLA-4-ΔE5R-hFlt3L-mOX40L-mIL-12-ΔB2R are Replication Competent in BSC40 Cells and B16-F10 Melanoma Cells

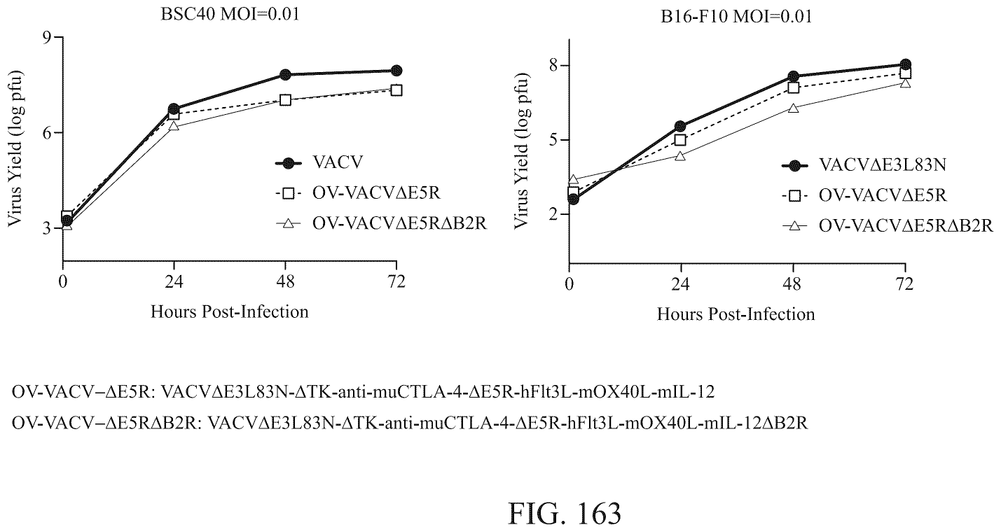

The replication capacities of VACVΔE3L83N-ΔTK-anti-muCTLA-4-ΔE5R-hFlt3L-mOX40L-mIL-12 (OV-VACVΔE5R) and VACVΔE3L83N-ΔTK-anti-muCTLA-4-ΔE5R-hFlt3L-mOX40L-mIL-12-ΔB2R (OV-VACVΔE5RΔB2R) in BSC40 cells and murine B16-F10 melanoma cells were determined by infecting them at a MOI of 0.01. Cells were collected at various time points post infection and viral yields (log pfu) were determined by titrating on BSC40 cells. FIG. 163 shows the graphs of viral yields plotted against hours post infection. Both VACVΔE3L83N-ΔTK-anti-muCTLA-4-ΔE5R-hFlt3L-mOX40L-mIL-12 and VACVΔE3L83N-ΔTK-anti-muCTLA-4-ΔE5R-hFlt3L-mOX40L-mIL-12-ΔB2R replicate efficiently in BSC40 cells and B16-F10 melanoma cells.

### Example 130: VACVΔE3L83N-ΔTK-Anti-muCTLA-4-ΔE5R-hFlt3L-mOX40L-mIL-12 (OV-VACVΔE5R) and VACVΔE3L83N-ΔTK-Anti-muCTLA-4-ΔE5R-hFlt3L-mOX40L-mIL-12-ΔB2R (OV-VACVΔE5RΔB2R) Infection of BMDCs Induces the Expression of Type I IFNB Gene and the Secretion of IFN-β Protein

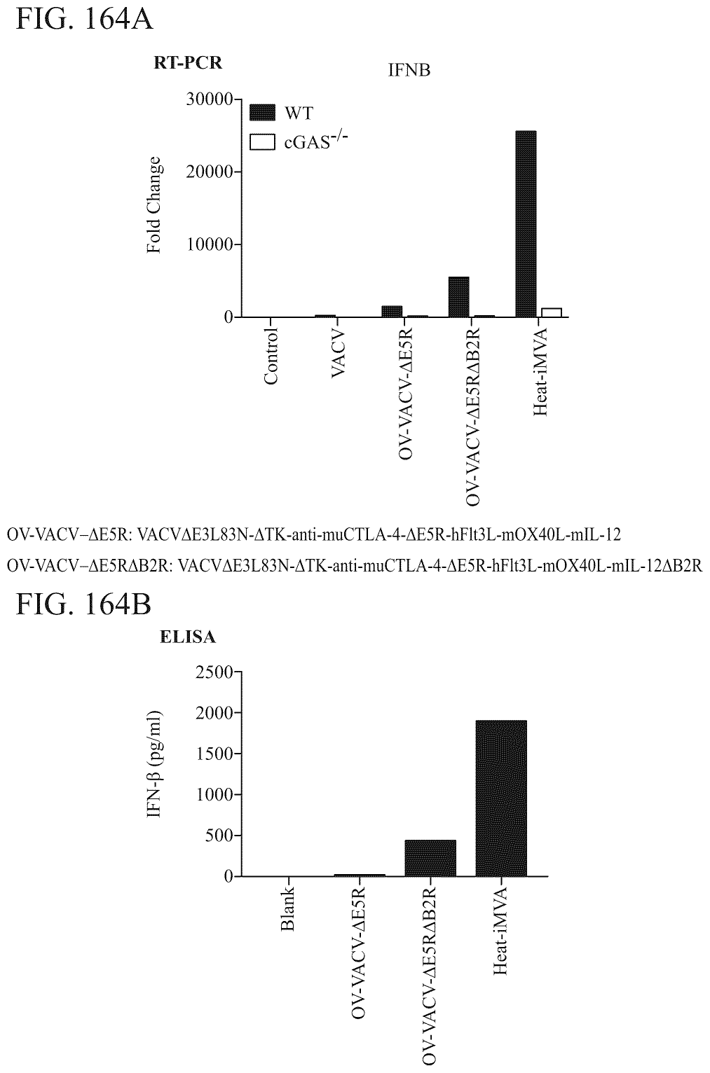

To test whether the recombinant viruses VACVΔE3L83N-ΔTK-anti-muCTLA-4-ΔE5R-hFlt3L-mOX40L-mIL-12 (OV-VACVΔE5R) and VACVΔE3L83N-ΔTK-anti-muCTLA-4-ΔE5R-hFlt3L-mOX40L-mIL-12-ΔB2R (OV-VACVΔE5RΔB2R) induce type I IFN production in BMDCs, WT and cGAS^{-/-} BMDCs were infected with these viruses at a MOI of 10. Cells were collected at 6 h post infection. RNAs were extracted. The IFNB gene expression levels were determined by quantitative RT-PCR analyses. Supernatants were collected at 24 h post infection and IFN-β levels in the supernatants were measured by ELISA. RT-PCR results showed that whereas WT VACV infection of BMDCs induced 225-fold higher levels of IFNB gene expression compared with no-treatment control, VACVΔE3L83N-ΔTK-anti-muCTLA-4-ΔE5R-hFlt3L-mOX40L-mIL-12 or VACVΔE3L83N-ΔTK-anti-muCTLA-4-ΔE5R-hFlt3L-mOX40L-mIL-12-ΔB2R infection induced 1546- and 5501-fold higher levels of IFNB gene expression, respectively, compared with no-infection control (FIG. 164A). The induction of IFNB gene expression by OV-VACVΔE5RΔB2R or OV-VACVΔE5R was abolished in cGAS^{-/-} BMDCs.

ELISA results showed that VACVΔE3L83N-ΔTK-anti-muCTLA-4-ΔE5R-hFlt3L-mOX40L-mIL-12 or VACVΔE3L83N-ΔTK-anti-muCTLA-4-ΔE5R-hFlt3L-mOX40L-mIL-12-ΔB2R infection of BMDCs induced IFN-β secretion from BMDCs (FIG. 164B). These results indicate that VACVΔE3L83N-ΔTK-anti-muCTLA-4-ΔE5R-hFlt3L-mOX40L-mIL-12 (OV-VACVΔE5R) or VACVΔE3L83N-ΔTK-anti-muCTLA-4-ΔE5R-hFlt3L-mOX40L-mIL-12-ΔB2R (OV-VACVΔE5RΔB2R) induced higher levels of IFNB gene expression and IFN-β protein secretion than WT VACV from BMDCs. In addition, OV-VACVΔE5RΔB2R infection of BMDCs induces higher levels of IFNB gene expression and IFN-b secretion compared with OV-VACVΔE5R (FIG. 164). Therefore, OV-VACVΔE5RΔB2R is more immune-activating compared with OV-VACVΔE5R.

### Example 131: Expression of Anti-muCTLA-4-hFlt3L,mOX40L in B16-F10 Melanoma Cells Infected with E3LΔ83N-ΔTK-Anti-muCTLA-4-ΔE5R-hFlt3L-mOX40L-mIL12 Virus

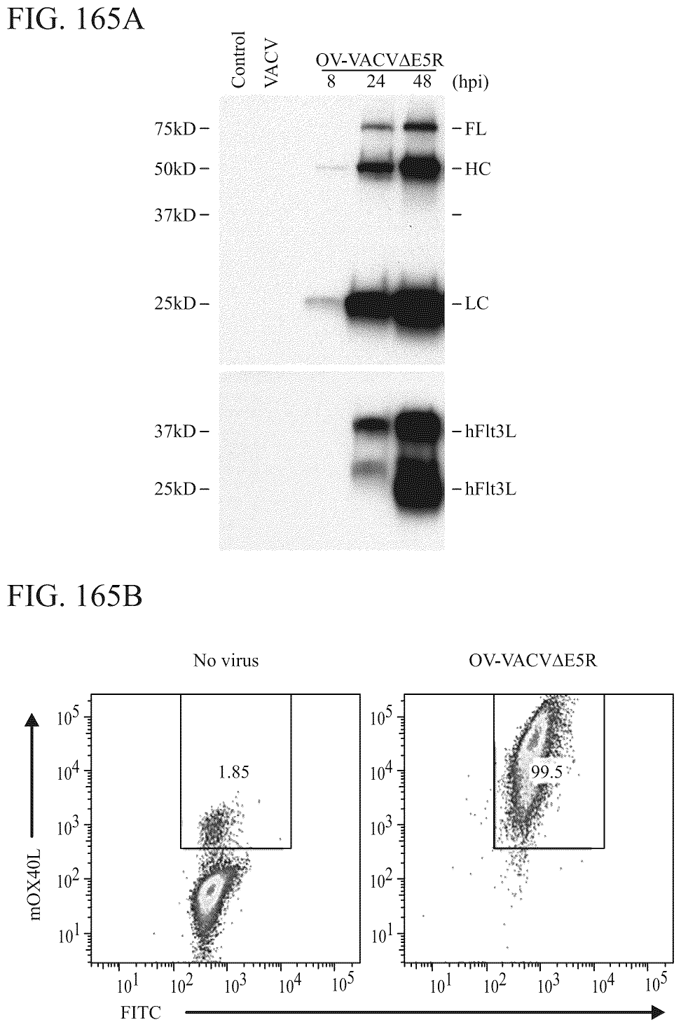

To determine whether VACVΔE3L83N-ΔTK-anti-muCTLA-4-ΔE5R-hFlt3L-mOX40L-mIL-12 (OV-VACVΔE5R) recombinant virus is capable of expressing desired transgenes, B16-F10 murine melanoma cells were infected with VACVΔE3L83N-ΔTK-anti-muCTLA-4-ΔE5R-hFlt3L-mOX40L-mIL-12 (OV-VACVΔE5R) at a MOI of 10. Cell lysates were collected at various times (8, 24, and 48 hours) post infection. Western blot analyses were performed to determine the expression of anti-muCTLA-4 and hFlt3L proteins. As shown in FIG. 165A, there were abundant expressions of anti-muCTLA-4 antibody and hFlt3L in B16-F10 cells infected with VACVΔE3L83N-ΔTK-anti-muCTLA-4-ΔE5R-hFlt3L-mOX40L-mIL-12 viruses. B16-F10 murine melanoma cells were also infected with VACVΔE3L83N-ΔTK-anti-muCTLA-4-ΔE5R-hFlt3L-mOX40L-mIL-12 at a MOI of 10, and the expression of mOX40L on cell surface were determined by FACS analysis. As shown in FIG. 165B, 99.5% of infected cells expressed mOX40L on cell surface. These results demonstrate that the recombinant poxvirus of the present technology have the capacity to express specific transgenes of interest in infected cells.

### Example 132: Intratumorally Injected E3LΔ83N-ΔTK-Anti-muCTLA-4-ΔE5R-hFlt3L-mOX40L-mIL-12 Virus has the Capacity to Express Desired Transgenes in Implanted Tumors In Vivo

A unilateral tumor implantation model was used to assess whether recombinant viruses can express specific transgenes in implanted tumors in vivo. B16-F10 melanoma cells (5×10⁵ cells) were intradermally implanted into the shaved skin on the right flank of C57BL/6J mice. Eight days after tumor implantation the tumors (about 4 mm in diameter) were injected with VACVΔE3L83N-ΔTK-anti-muCTLA-4-ΔE5R-hFlt3L-mOX40L-mIL-12 virus. Tumor samples were collected at 48 hours after virus injection. Western blot analyses showed that mIL-12 was detected in tumors treated with the recombinant virus expressing mIL-12, but not in PBS-treated tumors (FIG. 165C). These results demonstrate that the recombinant VACVΔE3L83N-ΔTK-anti-muCTLA-4-ΔE5R-hFlt3L-mOX40L-mIL-12 virus can express desired specific transgenes in injected tumors in vivo.

### Example 133: Secretion of mIL-12 from Murine B16-F10 Melanoma Cells, 4T1 Breast Cancer Cells, and MC38 Colon Cancer Cells Via Infection of E3LΔ83N-ΔTK-Anti-muCTLA-4-ΔE5R-hFlt3L-mOX40L-mIL-12 Viruses

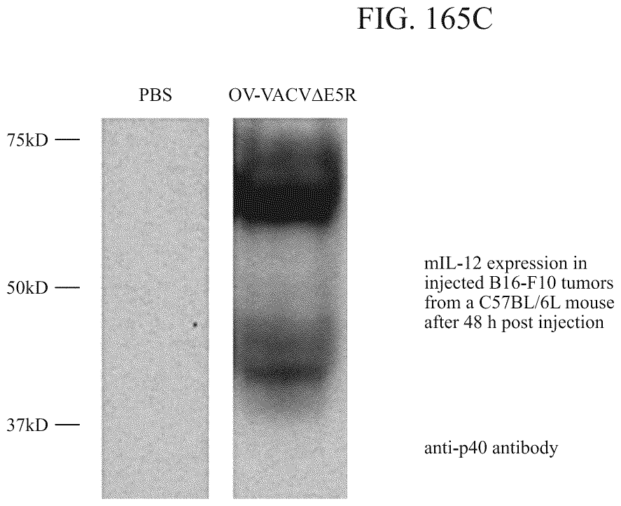

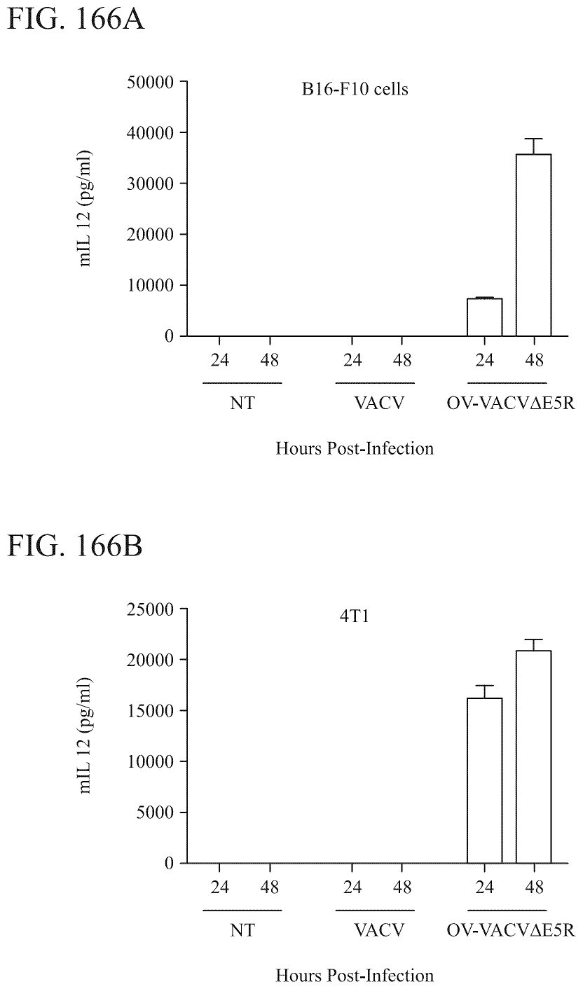

To examine whether recombinant viruses infected cells are capable of secreting desired proteins, B16-F10 murine melanoma cells, 4T1 breast cancer cells, and MC38 colon cancer cells were mock infected, or infected with VACV or E3LΔ83N-ΔTK-anti-muCTLA-4-ΔE5R-hFlt3L-mOX40L-mIL-12 (OV-VACVΔE5R) at a MOI of 10. The supernatant was collected at 24 and 48 hours after infection. ELISA was used to measure the concentration of secreted mIL-12 in the supernatant. As shown in FIG. 166, there is high levels of mIL-12 protein in the supernatants of E3LΔ83N-ΔTK-anti-muCTLA-4-ΔE5R-hFlt3L-mOX40L-mIL-12 (OV-VACVΔE5R) virus infected B16-F10 melanoma cells (FIG. 166A), 4T1 breast cancer cells (FIG. 166B), and MC38 colon cancer cells (FIG. 166C). These results demonstrate that OV-VACVΔE5R infection of tumor cells leads to mIL-12 release. To avoid mIL-12 toxicity, extracellular matrix tag were inserted to the C-terminus of IL12 p30 subunit. The coding sequence of p40 and p30 subunits of mIL12 is separated by a furin cleavage site followed by a Pep2A sequence. The C-terminus of p30 subunit is tagged with a matrix binding sequence.

### Example 134: Intratumoral Injection of E3LΔ83N-ΔTK-Anti-muCTLA-4-ΔE5R-hFlt3L-mOX40L-mIL-12 (OV-VACVΔE5R) is More Effective than HT-iMVA in a Bilateral B16-F10 Tumor Implantation Model

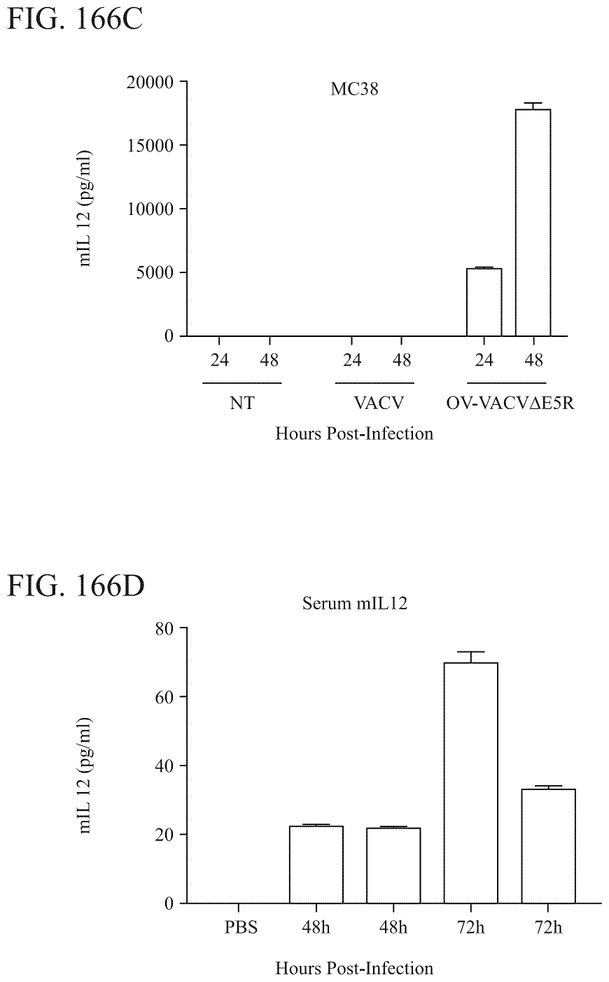

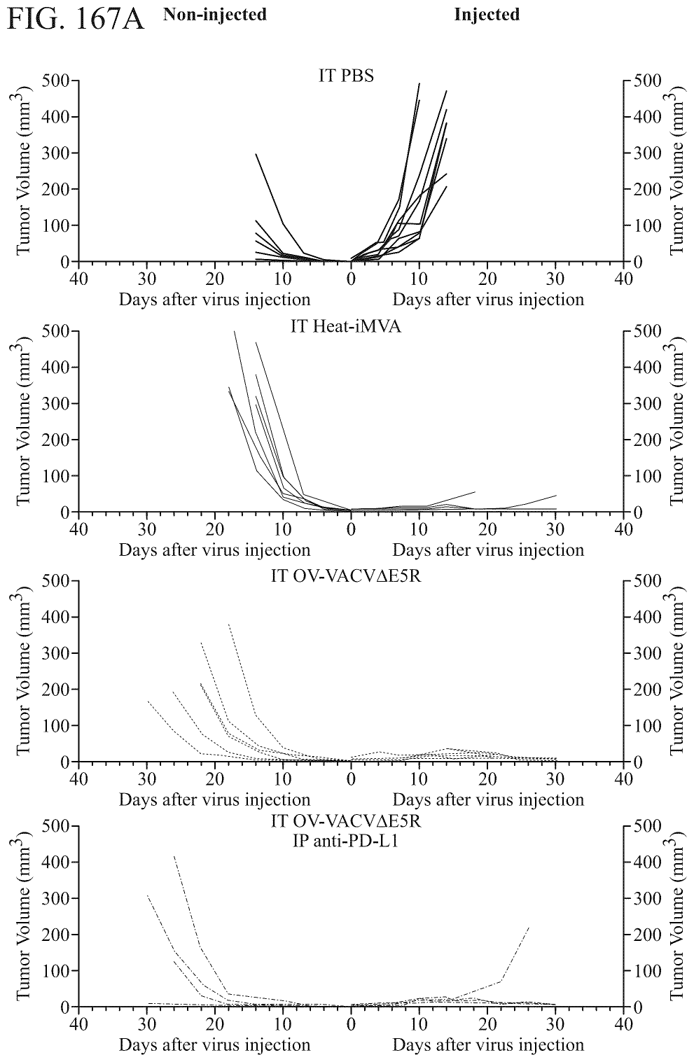

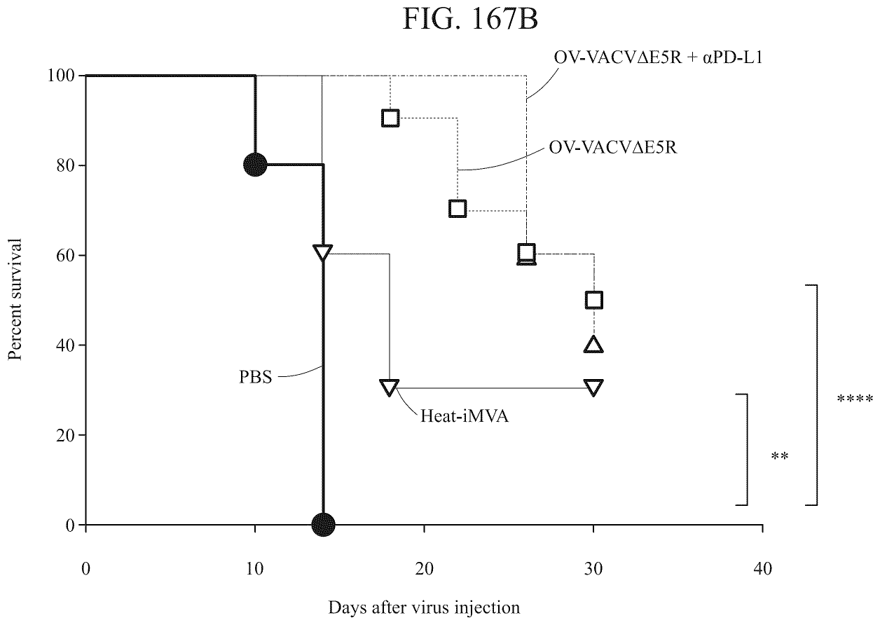

To test the in vivo tumor killing activities of the recombinant virus E3LΔ83N-ΔTK-anti-muCTLA-4-ΔE5R-hFlt3L-mOX40L-mIL-12, a bilateral tumor implantation model was used. B16-F10 melanoma cells were implanted intradermally into the shaved skin on the right (5×10⁵ cells) and left (1×10⁵ cells) flanks of a C57BL/6J mouse. After 7 to 8 days post implantation, the larger tumors on the right flank (about 3 mm or larger in diameter) were intratumorally injected twice per week with PBS, HT-iMVA, E3LΔ83N-ΔTK-anti-muCTLA-4-ΔE5R-hFlt3L-mOX40L-mIL-12, or E3LΔ83N-ΔTK-anti-muCTLA-4-ΔE5R-hFlt3L-mOX40L-mIL-12 plus intraperitoneal (IP) injection of anti-PD-L1 antibody (250 μg/mouse). Mice were monitored for survival and the tumor sizes were measured twice a week. FIG. 167A shows the tumor volume and FIG. 167B shows the Kaplan-Meier survival curve of the experiment. FIG. 167B shows that mice with PBS mock-treated tumors grew very quickly and the mice died with a median survival of 14 days. The injection of HT-iMVA into the tumors extended the median survival day to 18 days. Injection of E3LΔ83N-ΔTK-anti-muCTLA-4-ΔE5R-hFlt3L-mOX40L-mIL-12 increase the median survival to 30 days. Intratumoral injection of E3LΔ83N-ΔTK-anti-muCTLA-4-ΔE5R-hFlt3L-mOX40L-mIL-12 plus IP injection of anti-muPD-L1 antibody (250 μg/mouse) also increased the median survival to 30 days. FIG. 167A demonstrate the measured tumor volume over time for injected tumors and non-injected tumors. These results demonstrate that the expression of anti-muCTLA-4, hFlt3L, mOX40L, and mIL-12 by the engineered E3LΔ83N-ΔTK-anti-muCTLA-4-ΔE5R-hFlt3L-mOX40L-mIL-12 virus is capable of reducing tumor volume and slowing tumor growth in both injected and non-injected tumors, thereby demonstrating an abscopal effect. Accordingly, these results demonstrate that the recombinant poxviruses of the present technology are useful in methods for treating solid tumors.

### Example 135: Intratumoral Delivery of E3LΔ83N-ΔTK-Anti-muCTLA-4-ΔE5R-hFlt3L-mOX40L-mIL-12ΔB2R Induces Stronger Antitumor Systemic T Cell Responses Compared with E3LΔ83N-ΔTK-Anti-muCTLA-4-ΔE5R-hFlt3L-mOX40L-mIL-12 or Heat-iMVA

Infection of BMDCs with E3LΔ83N-ΔTK-anti-muCTLA-4-ΔE5R-hFlt3L-mOX40L-mIL-12-ΔB2R induces stronger type I IFN production compared with E3LΔ83N-ΔTK-anti-muCTLA-4-ΔE5R-hFlt3L-mOX40L-mIL-12. Without being bound by theory, it is hypothesized that IT delivery of E3LΔ83N-ΔTK-anti-muCTLA-4-ΔE5R-hFlt3L-mOX40L-mIL-12-ΔB2R virus would induce stronger antitumor immune responses. To testing whether further deletion of B2R from E3LΔ83N-ΔTK-anti-muCTLA-4-ΔE5R-hFlt3L-mOX40L-mIL-12 will enhance the antitumor efficacy, B16-F10 melanoma cells were implanted intradermally to the left and right flanks of C57B/6J mice (5×10⁵ to the right flank and 2.5×10⁵ to the left flank). Seven days post tumor implantation, 2×10⁷ pfu of either E3LΔ83N-ΔTK-anti-muCTLA-4-ΔE5R-hFlt3L-mOX40L-mIL-12-ΔB2R, E3LΔ83N-ΔTK-anti-muCTLA-4-ΔE5R-hFlt3L-mOX40L-mIL-12, or an equivalent amount of Heat-iMVA, or PBS was intratumorally injected into the larger tumors on the right flank twice, three days apart. Spleens were harvested at 3 days post second injection, ELISPOT analyses were performed to evaluate tumor-specific T cells in the spleens. ELISPOT assay was performed by co-culturing irradiated B16-F10 cells (150,000) and splenocytes (1,000,000) in a 96-well plate. FIG. 168A shows a graph of IFN-γf spots per 1,000,000 splenocytes. FIG. 168B shows the image of ELISPOT of triplicate samples of combined splenocytes from mice in the same treatment group. These results show that IT injection of E3LΔ83N-ΔTK-anti-muCTLA-4-ΔE5R-hFlt3L-mOX40L-mIL-12-ΔB2R generated the strongest antitumor T cell responses in the spleens of treated mice compared with either E3LΔ83N-ΔTK-anti-muCTLA-4-ΔE5R-hFlt3L-mOX40L-mIL-12 or Heat-iMVA.

### Example 136: Generation of the Recombinant Vaccinia Viruses with TK-E3L83N-E5R Deletions and Expressing Anti-huCTLA-4, hFlt3L, hOX40L, and hIL-12 (VACVΔE3L83N-ΔTK-Anti-huCTLA-4-ΔE5R-hFlt3L-hOX40L-hIL-12)

This example describes the generation of the recombinant VACVΔE3L83N-ΔTK-anti-huCTLA-4-ΔE5R-hFlt3L-hOX40L-IL-12 virus.

FIG. 169 shows the stepwise strategy to generate recombinant VACVΔE3L83NΔTKΔE5R virus expressing anti-huCTLA-4, hFlt3L, hOX40L, and hIL12 proteins through homologous recombination first at the TK loci and then at the E5R loci of the VACVΔE3L83N genome. pCB-anti-huCTLA-4 gpt vector was used to insert a single expression cassette to express the anti-huCTLA-4 antibody heavy and light chains under the control of the vaccinia virus synthetic early and late promoter (PsE/L). Homologous recombination that occurred at the TK-L and TK-R sites results in the insertion of expression cassette of anti-huCTLA-4 antibody into TK locus of the VACVΔE3L83N genome, generating the recombinant virus VACVΔE3L83N-ΔTK-anti-huCTLA-4. pUC57-hFlt3L-hOX40L-hIL12 mCherry vector was used to insert a single expression cassette designed to express both hFlt3L-hOX40L fusion protein and hIL12 protein separately using the vaccinia viral synthetic early and late promoter (PsE/L) in opposite direction. The coding sequence of the hFlt3L-hOX40L was separated by a cassette including a furin cleavage site followed by a Pep2A sequence. Homologous recombination at the E4L and E6R loci resulted in the insertion of the expression cassette for hFlt3L-hOX40L and hIL12 into the E5L locus of VACVΔE3L83N-ΔTK-anti-huCTLA-4 virus. BSC40 cells will be infected with VACVΔE3L83N at a MOI of 0.05 for 1 h, and then will be transfected with the plasmid pCB-anti-muCTLA-4 gpt. The infected cells will be collected at 48h after virus infection. Recombinant viruses will be selected through further culturing in selection medium including MPA, xanthine and hypoxanthine, and plaque purified. PCR analysis will be performed to verify the insertion of anti-muCTLA-4 gene into the TK locus. For inserting hFlt3L-hOX40L-hIL12 expression cassette into the E5R locus of VACVΔE3L83N-ΔTK-anti-huCTLA-4 virus, BSC40 cells will be infected with VACVΔE3L83N-ΔTK-anti-huCTLA-4 at a MOI of 0.05 for 1 h, and then will be transfected with the plasmid pUC57-hFlt3L-hOX40L-hIL12 mCherry. The infected cells will be collected at 48 h after virus infection. Recombinant viruses will be isolated through plaque purification by selecting mCherry positive virus plaques. PCR analysis will performed to verify the insertion of hFlt3L-hOX40L-hIL12gene into the E5R locus. The vaccinia B2R, B18R, and WR199 genes will further be deleted to enhance the innate immune response of this virus.

### Example 137: Generation of the Recombinant Myxoma Viruses MyxomaΔM063R and MyxomaΔM064R

Myxoma M063R (M63R) and M064R (M64R) are orthologs of vaccinia C7. To test whether deletion of M063R or M064R from the parental genome improves IFN induction capacity of Myxoma virus, the inventors generated MyxomaΔM063R and MyxomaΔM064R through homologous recombinations at the homology arms of the transfected plasmids and the parental myxoma viral genome (FIG. 170). pUC57 vector is used to insert a single expression cassette designed to express EGFP using the vaccinia viral synthetic early and late promoter (PsE/L). Homologous recombination that occurred at the M062R and M064R loci results in the insertion of expression cassette for EGFP and the deletion of M063. Similarly, homologous recombination that occurred at the M063R and M065R loci results in the insertion of expression cassette for EGFP and the deletion of M064. Briefly, BGMK cells were infected with the parental myxoma virus MyxomaΔM127-mcherry (Myxoma-mcherry) at a MOI of MOI of 0.05 for 1 h, and then were transfected with the plasmid DNAs described above. The infected cells were collected at 48 h. Recombinant viruses were identified by their green fluorescence with the insertion of EGFP into the M063 or M064 loci and by mCherry in the parental virus. The double positive clones were then purified 6-8 times on BGMK cells. PCR analysis were performed to confirm that recombinant viruses MyxomaΔM063R (ΔM63R) and MyxomaΔM064R (ΔM064R) have lost M063R and M064R genes respectively.

### Example 138: MyxomaΔM063R and MyxomaΔM064R Induces Higher Levels of IFNB Gene Expression and IFN-□ D Protein Secretion in BMDCs Compared with the Parental Virus

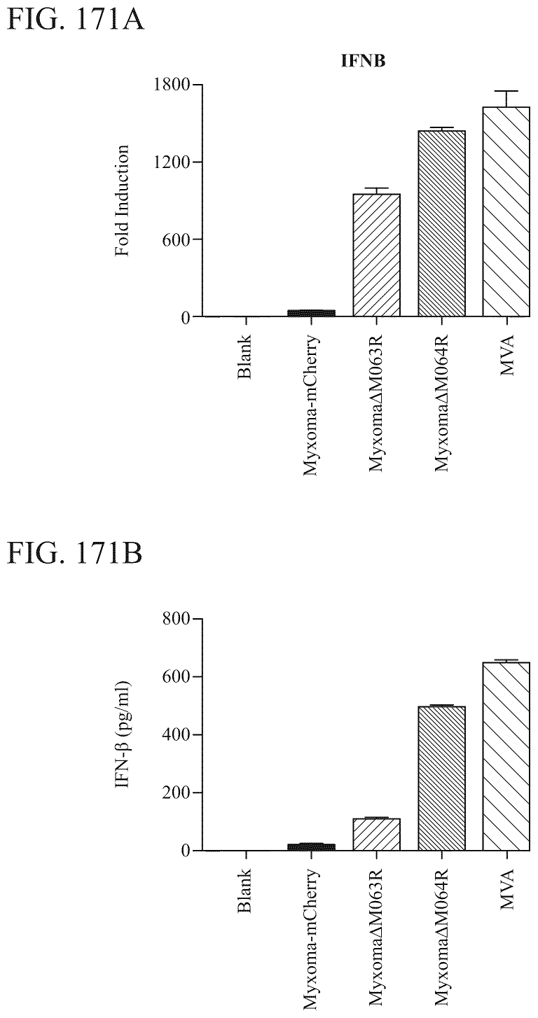

To test whether deleting the M063R gene or the M064R gene from the parental myxoma virus (Myxoma-mcherry) induces higher levels of type I IFN, BMDCs (1×10⁶) from WT C57BL/6J mice were infected with either Myxoma-mcherry, MyxomaΔM063R, MyxomaΔM064R, or MVA at a MOI of 10. Cells were collected at 6 h post infection. Supernatants were collected at 19 h post infection. IFNB gene expression was determined by RT-PCR. The results show that both MyxomaΔM063R and MyxomaΔM064R induce higher levels of IFNB gene expression compared with Myxoma-mcherry, but lower levels of IFNB compared with MVA in BMDCs (FIG. 171A).

IFN-□ protein levels in the supernatants were determined by ELISA (FIG. 171B).

The results show that both MyxomaΔM064 and MyxomaΔM063 infection of BMDCs induce higher levels of IFN-□ protein secretion compared with Myxoma-mcherry. These results indicate that removing the vaccinia C7 orthologs from myxoma virus further improves IFN induction capacity of the virus.

### Example 139: Intratumoral Delivery of Myxoma-Mcherry or MyxomaΔM064R Induces CD8 and CD4 T Cell Activation in Injected Tumors Two Days after Treatment in a B16-F10 Melanoma Model

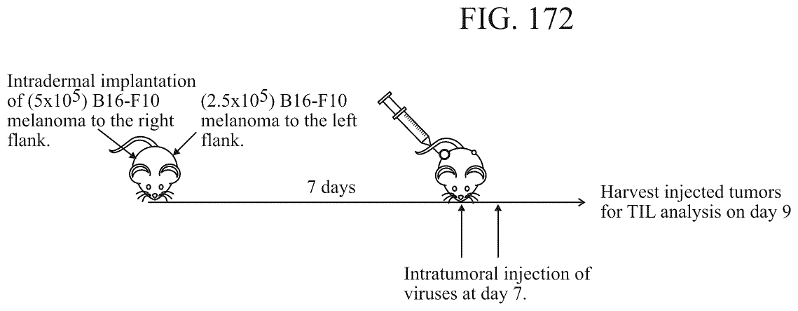

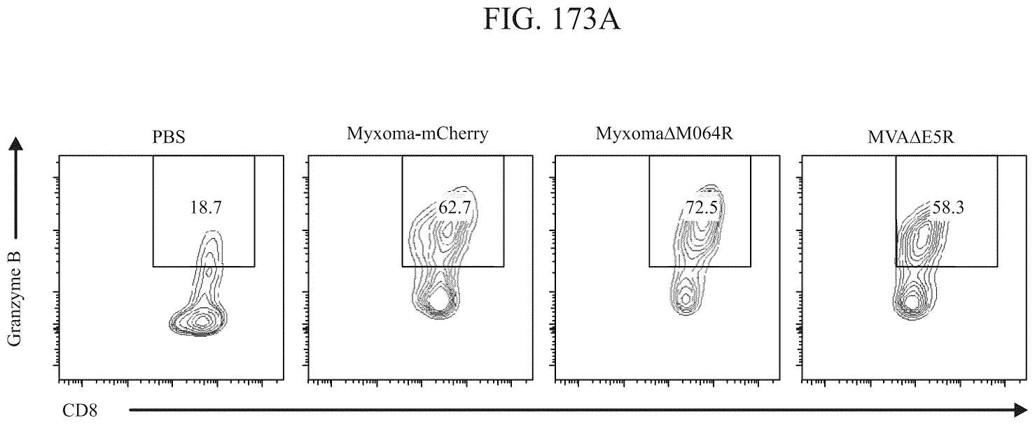

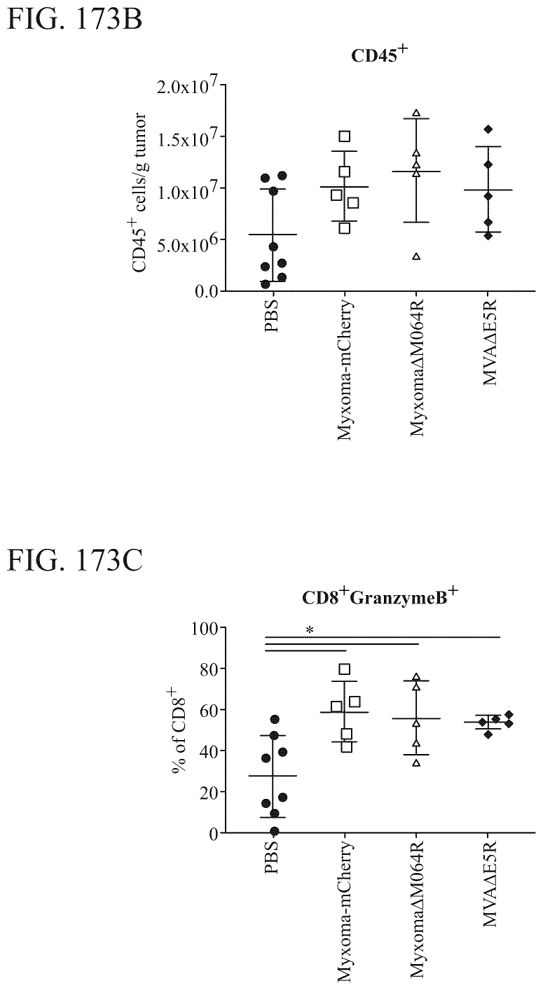

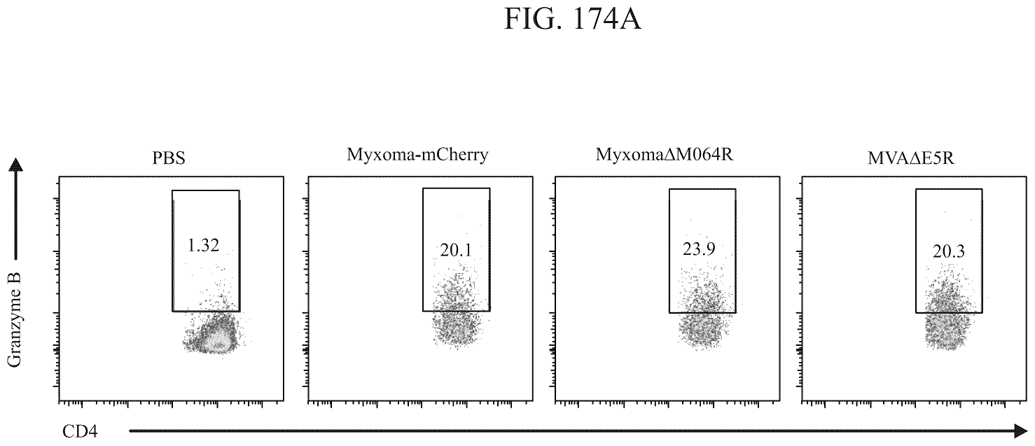

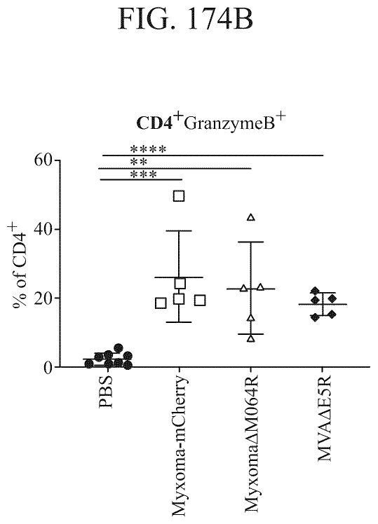

To assess whether IT delivery of the parental myxoma virus or MyxomaΔM064R alters immune-suppressive tumor microenvironment, the following experiment was performed. Briefly, B16-F10 melanoma cells were implanted intradermally into the shaved skin on the right (5×10⁵ cells) and left (2.5×10⁵ cells) flanks of C57BL/6J mice. Seven days post implantation, the larger tumors on the right flank were injected with 4×10⁷ pfu of Myxoma-mcherry, MyxomaΔM064, or MVAΔE5R, or PBS just once. Two days post injection, tumors were harvested and cells were processed for surface labeling with anti-CD3, CD45, CD4, CD8, and also for intracellular Granzyme B staining. The live immune cell infiltrates in the tumors were analyzed by FACS (FIG. 172). FIG. 173A shows representative dot plots of Granzyme B⁺CD8⁺ T cells. IT Myxoma-mcherry, MyxomAM064, or MVAΔE5R results in the activation of CD8⁺ T cells in the injected tumors. FIG. 173B shows that IT Myxoma-mcherry, MyxomAM064, or MVAΔE5R results in higher numbers of CD45⁺ cells in the injected tumors compared with those treated with PBS. FIG. 173C shows IT Myxoma-mcherry, MyxomaΔM064, or MVAΔE5R results in higher percentages of Granzyme B⁺CD8⁺ cells in the injected tumors compared with those treated with PBS. FIG. 174A shows representative dot plots of Granzyme B⁺CD4⁺ T cells. IT Myxoma-mcherry, MyxomAM064, or MVAΔE5R results in the activation of CD4⁺ T cells in the injected tumors. FIG. 174B shows that IT Myxoma-mcherry, MyxomaΔM064, or MVAΔE5R results in higher percentages of Granzyme B⁺CD4⁺ cells in the injected tumors compared with those treated with PBS. These results demonstrate that the recombinant poxviruses of the present technology are useful in methods for treating solid tumors.

### Equivalents

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds, compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

Other embodiments are set forth within the following claims.

What is claimed is:

1. A modified vaccinia Ankara (MVA) virus genetically engineered to comprise a mutant E5R gene (MVAΔE5R), wherein the virus is selected from the group consisting of:
(i) an MVAΔE5R virus comprising a heterologous nucleic acid molecule encoding human OX40 ligand (hOX40L) and a heterologous nucleic acid molecule encoding human Fms-like tyrosine kinase 3 ligand (hFlt3L) (MVAΔE5R-hOX40L-hFlt3L);
(ii) an MVAΔE5R virus comprising a heterologous nucleic acid molecule encoding hFlt3L, a heterologous nucleic acid molecule encoding hOX40L, a heterologous nucleic acid molecule encoding hIL-12, a mutant E3L gene (ΔE3L), a mutant WR199 gene (ΔWR199), and a mutant C12L gene (ΔC12L) (MVAΔE3ΔE5RL-hFlt3L-hOX40LΔWR199-hIL-12ΔC12L);
(iii) an MVAΔE5R virus comprising a heterologous nucleic acid molecule encoding hFlt3L, a heterologous nucleic acid molecule encoding hOX40L, a heterologous nucleic acid molecule encoding hIL-12, ΔE3L, a ΔWR199, a ΔC12L, and a heterologous nucleic acid molecule encoding human interleukin 18 (hIL-18) (MVAΔE3ΔE5RL-hFlt3L-hOX40LΔWR199-hIL-12ΔC12L-hIL-18);
(iv) an MVAΔE5R virus comprising a heterologous nucleic acid molecule encoding hOX40L (MVAΔE5R-hOX40L);
(v) an MVAΔE5R virus comprising a heterologous nucleic acid molecule encoding hFlt3L (MVAΔE5R-hFlt3L);
(vi) an MVAΔE5R virus comprising a ΔE3L and a heterologous nucleic acid molecule encoding hOX40L (MVAΔE3LΔE5R-hOX40L);
(vii) an MVAΔE5R virus comprising a ΔE3L, a heterologous nucleic acid molecule encoding hFlt3L, and a heterologous nucleic acid molecule encoding hOX40L (MVAΔE3LΔE5R-hFlt3L-hOX40L);
(viii) an MVAΔE5R virus comprising a ΔE3L, a heterologous nucleic acid molecule encoding hFlt3L, a heterologous nucleic acid molecule encoding hOX40L, and a ΔWR199 (MVAΔE3LΔE5R-hFlt3L-hOX40LΔWR199); and
(ix) an MVAΔE5R virus comprising an E5R gene deletion.

2. The MVAΔE5R virus of claim 1, wherein the virus further comprises one or more heterologous nucleic acid molecules encoding one or more of hOX40L, hFlt3L, hIL-2, hIL-12, hIL-15, hIL-15/IL-15Rα, hIL-18, hIL-21, anti-huCTLA-4, anti-huPD-1, anti-huPD-L1, GITRL, 4-1BBL, or CD40L, and/or a deletion of any one or more of thymidine kinase, C7, E3LΔ83N, B2R, WR200, K7R, C12L, B8R, B14R, N1L, C11R, K1L, M1L, N2L, or WR199.

3. The MVAΔE5R virus of claim 2, wherein, when the virus further comprises one or more heterologous nucleic acids, the heterologous nucleic acids are expressed from within one or more viral genes selected from the group consisting of the thymidine kinase (TK) gene, the C7 gene, the C11 gene, the K3 gene, the F1 gene, the F2 gene, the F4 gene, the F6 gene, the F8 gene, the F9 gene, the F11 gene, the F14.5 gene, the J2 gene, the A46 gene, the E3L gene, the WR200 gene, the E5R gene, the K7R gene, the C12L (IL18BP) gene, the B8R gene, the B14R gene, the N1L gene, the K1L gene, the C16 gene, the M1L gene, the N2L gene, and the WR199 gene.

4. The MVAΔE5R virus of claim 1, wherein the mutant E5R gene comprises replacement of at least a portion of the E5R gene with one or more gene cassettes comprising one or more heterologous nucleic acid molecules.

5. An immunogenic composition comprising the MVAΔE5R virus of claim 1.

6. The immunogenic composition of claim 5, further comprising a pharmaceutically acceptable carrier and/or adjuvant.

7. A nucleic acid sequence encoding the MVAΔE5R virus of claim 1.

8. A kit comprising the MVAΔE5R virus of claim 1 and instructions for use thereof.

9. A method for treating a tumor in a subject in need thereof, the method comprising delivering to a tumor a composition comprising an effective amount of the MVAΔE5R virus of claim 1.

10. The method of claim 9, wherein the treatment comprises one or more of the following: inducing an immune response in the subject against the tumor or enhancing or promoting an ongoing immune response against the tumor in the subject, reducing the size of the tumor, eradicating the tumor, inhibiting the growth of the tumor, inhibiting metastatic growth of the tumor, inducing apoptosis of tumor cells, or prolonging survival of the subject.

11. The method of claim 9, wherein the composition is administered by intratumoral or intravenous injection or a simultaneous or sequential combination of intratumoral and intravenous injection.

12. The method of claim 9, wherein the tumor is melanoma, colon, breast, bladder, prostate carcinoma, or Extramammary Paget disease (EMPD).

13. The method of claim 9, wherein the method further comprises separately, sequentially, or simultaneously administering to the subject one or more agents selected from: one or more immune checkpoint blocking agents; one or more anti-cancer drugs, fingolimod (FTY720); and any combination thereof.

14. The method of claim 13, wherein:
the one or more immune checkpoint blocking agents is selected from the group consisting of anti-PD-L1 antibody, anti-PD-1 antibody, anti-CTLA-4 antibody, ipilimumab, nivolumab, pidilizumab, lambrolizumab, pembrolizumab, atezolizumab, avelumab, durvalumab, MPDL3280A, BMS-936559, MEDI-4736, MSB 00107180, LAG-3, TIM3, B7-H3, B7-H4, TIGIT, AMP-224, MDX-1105, arelumab, tremelimumab, IMP321, MGA271, BMS-986016, lirilumab, urelumab, PF-05082566, IPH2101, MEDI-6469, CP-870,893, Mogamulizumab, Varlilumab, Galiximab, AMP-514, AUNP 12, Indoximod, NLG-919, INCB024360, CD80, CD86, ICOS, DLBCL inhibitors, BTLA, PDR001, and any combination thereof; and/or
the one or more anti-cancer drugs is selected from the group consisting of a Mek inhibitor (U0126, selumitinib (AZD6244), PD98059, trametinib, cobimetinib), an EGFR inhibitor (lapatinib (LPN), erlotinib (ERL)), a HER2 inhibitor (lapatinib (LPN), Trastuzumab), a Raf inhibitor (sorafenib (SFN)), a BRAF inhibitor (dabrafenib, vemurafenib), an anti-OX40 antibody, a GITR agonist antibody, an anti-CSFR antibody, a CSFR inhibitor, paclitaxel, TLR9 agonist CpG, and a VEGF inhibitor (Bevacizumab), and any combination thereof.

15. The method of claim 14, wherein the one or more immune checkpoint blocking agents comprises anti-PD-L1 antibody, anti-PD-1 antibody, or anti-CTLA-4 antibody.

16. The method of claim 13, wherein the combination of the MVAΔE5R virus with the immune checkpoint blocking agent, anti-cancer drug, and/or fingolimod (FTY720) has a synergistic effect in the treatment of the tumor as compared to administration of either the MVAΔE5R virus or of the immune checkpoint blocking agent, anti-cancer drug, or fingolimod (FTY720) alone.

17. A vaccinia virus (VACV) genetically engineered to comprise a mutant E5R gene (VACVΔE5R), wherein the virus is selected from the group consisting of:
(i) a VACVΔE5R virus comprising a heterologous nucleic acid molecule encoding an anti-huCTLA-4 antibody, a heterologous nucleic acid molecule encoding hFlt3L, a heterologous nucleic acid molecule encoding hOX40L, a heterologous nucleic acid molecule encoding hIL-12, an E3LΔ83N deletion, and a ΔTK (VACVE3LΔ83N-ΔTK-anti-huCTLA-4-ΔE5R-hFlt3L-hOX40L-hIL-12);
(ii) a VACVΔE5R virus comprising a heterologous nucleic acid molecule encoding an anti-huCTLA-4 antibody, a heterologous nucleic acid molecule encoding hFlt3L, a heterologous nucleic acid molecule encoding hOX40L, a heterologous nucleic acid molecule encoding hIL-12, an E3LΔ83N deletion, a ΔTK, and a ΔWR199 (VACVE3LΔ83N-ΔTK-anti-huCTLA-4-ΔE5R-hFlt3L-hOX40L-hIL-12ΔWR199);
(iii) a VACVΔE5R virus comprising a heterologous nucleic acid molecule encoding an anti-huCTLA-4 antibody, a heterologous nucleic acid molecule encoding hFlt3L, a heterologous nucleic acid molecule encoding hOX40L, a heterologous nucleic acid molecule encoding hIL-12, an E3LΔ83N deletion, a ΔTK, a ΔWR199, and a WR200 deletion (ΔWR200) (VACVE3LΔ83N-ΔTK-anti-huCTLA-4-ΔE5R-hFlt3L-hOX40L-hIL-12ΔWR199ΔWR200);
(iv) a VACVΔE5R virus comprising a heterologous nucleic acid molecule encoding an anti-huCTLA-4 antibody, a heterologous nucleic acid molecule encoding hFlt3L, a heterologous nucleic acid molecule encoding hOX40L, a heterologous nucleic acid molecule encoding hIL-12, an E3LΔ83N deletion, a thymidine kinase deletion (ΔTK), and a ΔC12L (VACVE3LΔ83N-ΔTK-anti-huCTLA-4-ΔE5R-hFlt3L-hOX40L-hIL-12ΔC12L);
(v) a VACVΔE5R virus comprising a heterologous nucleic acid molecule encoding an anti-huCTLA-4 antibody, a heterologous nucleic acid molecule encoding hFlt3L, a heterologous nucleic acid molecule encoding hOX40L, a heterologous nucleic acid molecule encoding hIL-12, an E3LΔ83N deletion, a ΔTK, a ΔWR199, a ΔWR200, and a ΔC12L (VACVE3LΔ83N-ΔTK-anti-huCTLA-4-ΔE5R-hFlt3L-hOX40L-hIL-12ΔWR199ΔWR200ΔC12L);
(vi) a VACVΔE5R virus comprising a heterologous nucleic acid molecule encoding an anti-huCTLA-4 antibody, a heterologous nucleic acid molecule encoding hFlt3L, a heterologous nucleic acid molecule encoding hOX40L, a heterologous nucleic acid molecule encoding hIL-12, an E3LΔ83N deletion, a ΔTK, a ΔWR199, a ΔWR200, a ΔC12L, and a heterologous nucleic acid molecule encoding hIL-18 (VACVE3LΔ83N-ΔTK-anti-huCTLA-4-ΔE5R-hFlt3L-hOX40L-hIL-12ΔWR199ΔWR200ΔC12L-hIL18); and
(vii) a VACVΔE5R virus comprising an E5R gene deletion.

18. A method for treating a tumor in a subject in need thereof, the method comprising delivering to a tumor a composition comprising an effective amount of the VACVΔE5R virus of claim 17.

19. A modified vaccinia Ankara (MVA) virus genetically engineered to comprise a mutant E3L gene (MVAΔE3L) and a heterologous nucleic acid molecule encoding hOX40L (MVAΔE3L-hOX40L), optionally wherein the MVAΔE3L virus comprises an E3L gene deletion.

20. A method for treating a tumor in a subject in need thereof, the method comprising delivering to a tumor a composition comprising an effective amount of the MVAΔE3L-hOX40L virus of claim 19.


SEQUENCE LISTING (1 sequence)

SEQ ID NO: 1 - DNA, 251 bp, Vaccinia virus
  source 1..251: mol_type = other DNA
gtaagattaaattaattataaaattatgtatataatattaattataaaattatgtatatgatttactaactttagttagataaattaataatacataaattttagtatattaatattataaattaataatacataaattttagtatattaatattatattttaaatatttatttagtgtctagaaaaaaatgtgtgaccaacgaccgtaggaaactctagagggtaagaaaaatcaatcgctttatagaga